(19) United States
(12) Patent Application Publication
(10) Pub. No.: US 2014/0080810 A1
(43) Pub. Date: Mar. 20, 2014

(54) Benzoxazepines as Inhibitors of PI3K/mTOR and Methods of Their Use and Manufacture

(71) Applicants: Kenneth D. Rice, San Rafael, CA (US); David Markby, San Francisco, CA (US)

(72) Inventors: Kenneth D. Rice, San Rafael, CA (US); David Markby, San Francisco, CA (US)

(73) Assignee: Exelixis, Inc., South San Francisco, CA (US)

(21) Appl. No.: 13/885,323

(22) PCT Filed: Nov. 15, 2011
(86) PCT No.: PCT/US11/60771

§ 371 Date: Nov. 12, 2013

Related U.S. Application Data
(60) Provisional application No. 61/413,954, filed on Nov. 15, 2010.

(51) Int. Cl. A61K 31/553; C12Q 1/48; C07D 471/04; C12Q 1/68; C07D 413/14; A61K 31/5377; G01N 33/574

(52) U.S. Cl. CPC A61K 31/553; A61K 31/5377; C12Q 1/485; G01N 33/5748; C12Q 1/6886; C07D 413/14; C07D 471/04
USPC 514/211.09; 514/233.2; 435/15; 435/7.4; 435/6.11; 435/287.1; 435/8; 506/9

(57) ABSTRACT

The invention is directed to Compounds of Formula I: (I) and pharmaceutically acceptable salts or solvates thereof, as well as methods of treating using the compounds, methods for screening for inhibitor compounds and methods for identifying treatment regimens.

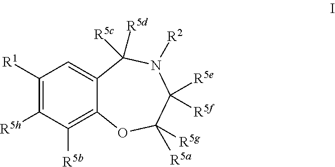

### CROSS-REFERENCE TO RELATED APPLICATION

This application claims the benefit of priority to U.S. Provisional Application No. 61/413,954, filed Nov. 15, 2010, which is incorporated herein by reference.

### SEQUENCE LISTING

This application incorporates by reference in its entirety the Sequence Listing entitled “EX10-028_SequenceListing.txt” (14.5 KB) which was created Nov. 15, 2011 and filed herewith on Nov. 15, 2011.

### BACKGROUND OF THE INVENTION

1. Field of the Invention

This invention relates to the field of protein kinases and inhibitors thereof. In particular, the invention relates to inhibitors of Phosphatidylinositol 3-kinase (PI3Kα) signaling pathways, screening assays to identify PI3Kα selective inhibitors, and methods for treating cancer patients with PI3Kα selective inhibitors, PI3Kβ selective inhibitors, mammalian target of rapamycin (mTOR) kinase inhibitors and combinations thereof.

2. Background of the Invention

Phosphatidylinositol 3-kinases (PI 3-kinases or PI3Ks) are a family of enzymes involved in cellular functions such as cell growth, proliferation, differentiation, motility, survival and intracellular trafficking, which in turn are involved in cancer. PI3Ks are a family of related intracellular signal transducer enzymes capable of phosphorylating the 3 position hydroxyl group of the inositol ring of phosphatidylinositol (PtdIns). Phosphatidylinositol 3-kinase is composed of an 85 kDa regulatory subunit and a 110 kDa catalytic subunit. The protein encoded by PI3KCA gene represents the catalytic subunit, which uses ATP to phosphorylate phosphatidylinositols (PtdIns), PtdIns4P and PtdIns(4,5)P2.

Phosphatidylinositol 3-kinase (PI3Kα), a dual specificity protein kinase, is composed of an 85 kDa regulatory subunit and a 110 kDa catalytic subunit. The protein encoded by this gene represents the catalytic subunit, which uses ATP to phosphorylate PtdIns, PtdIns4P and PtdIns(4,5)P2. PTEN, a tumor suppressor which inhibits cell growth through multiple mechanisms, can dephosphorylate PIP3, the major product of PI3KCA. PIP3, in turn, is required for translocation of protein kinase B (AKT1, PKB) to the cell membrane, where it is phosphorylated and activated by upstream kinases. The effect of PTEN on cell death is mediated through the PI3KCA/AKT1 pathway.

PI3Kα has been implicated in the control of cytoskeletal reorganization, apoptosis, vesicular trafficking, proliferation and differentiation processes. Increased copy number and expression of PI3KCA is associated with a number of malignancies such as ovarian cancer (Campbell et al., Cancer Res 2004, 64, 7678-7681; Levine et al., Clin Cancer Res 2005, 11, 2875-2878; Wang et al., Hum Mutat 2005, 25, 322; Lee et al., Gynecol Oncol 2005, 97, 26-34), cervical cancer, breast cancer (Bachman, et al. Cancer Biol Ther 2004, 3, 772-775; Levine, et al., supra; Li et al., Breast Cancer Res Treat 2006, 96, 91-95; Saal et al., Cancer Res 2005, 65, 2554-2559; Samuels and Velculescu, Cell Cycle 2004, 3, 1221-1224), colorectal cancer (Samuels, et al. Science 2004, 304, 554; Velho et al. Eur J Cancer 2005, 41, 1649-1654), endometrial cancer (Oda et al. Cancer Res. 2005, 65, 10669-10673), gastric carcinomas (Byun et al., Int J Cancer 2003, 104, 318-327; Li et al., supra; Velho et al., supra; Lee et al., Oncogene 2005, 24, 1477-1480), hepatocellular carcinoma (Lee et al., id.), small and non-small cell lung cancer (Tang et al., Lung Cancer 2006, 51, 181-191; Massion et al., Am J Respir Crit Care Med 2004, 170, 1088-1094), thyroid carcinoma (Wu et al., J Clin Endocrinol Metab 2005, 90, 4688-4693), acute myelogenous leukemia (AML) (Sujobert et al., Blood 1997, 106, 1063-1066), chronic myelogenous leukemia (CML) (Hickey and Cotter J Biol Chem 2006, 281, 2441-2450), and glioblastomas (Hartmann et al. Acta Neuropathol (Berl) 2005, 109, 639-642; Samuels et al., supra).

The mammalian target, mTOR, is a protein kinase that integrates both extracellular and intracellular signals of cellular growth, proliferation, and survival. Extracellular mitogenic growth factor signaling from cell surface receptors and intracellular pathways that convey hypoxic stress, energy and nutrient status all converge at mTOR. mTOR exists in two distinct complexes: mTOR complex 1 (mTORC1) and mTOR complex 2 (mTORC2). mTORC1 is a key mediator of transcription and cell growth (via its substrates p70S6 kinase and 4E-BPI) and promotes cell survival via the serum and glucocorticoid-activated kinase SGK, whereas mTORC2 promotes activation of the pro-survival kinase AKT. Given its central role in cellular growth, proliferation and survival, it is perhaps not surprising that mTOR signaling is frequently dysregulated in cancer and other diseases (Bjornsti and Houghton Rev Cancer 2004, 4(5), 335-48; Houghton and Huang Microbiol Immunol 2004, 279, 339-59; Inoki, Corradetti et al. Nat Genet 2005, 37(1), 19-24).

mTOR is a member of the PIKK (PI3K-related Kinase) family of atypical kinases which includes ATM, ATR, and DNAPK, and its catalytic domain is homologous to that of PI3K. Dyregulation of PI3K signaling is a common function of tumor cells. In general, mTOR inhibition may be considered as a strategy in many of the tumor types in which PI3K signaling is implicated such as those discussed below.

Inhibitors of mTOR may be useful in treating a number of cancers, including the following: breast cancer (Nagata, Lan et al., Cancer Cell 2004, 6(2), 117-27; Pandolfi N Engl J Med 2004, 351(22), 2337-8; Nahta, Yu et al. Nat Clin Pract Oncol 2006, 3(5), 269-280); antle cell lymphoma (MCL) (Dal Col, Zancai et al. Blood 2008, 111(10), 5142-51); renal cell carcinoma (Thomas, Tran et al. Nat Med 2006, 12(1), 122-7; Atkins, Hidalgo et al. J Clin Oncol 2004, 22(5), 909-18; Motzer, Hudes et al. J Clin Oncol 2007, 25(25), 3958-64); acute myelogenous leukemia (AML) (Sujobert, Bardet et al. Blood 2005, 106(3), 1063-6; Billottet, Grandage et al. Oncogene 2006, 25(50), 6648-6659; Tamburini, Elie et al. Blood 2007, 110(3), 1025-8); chronic myelogenous leukemia (CML) (Skorski, Bellacosa et al. Embo J 1997, 16(20), 6151-61; Bai, Ouyang et al. Blood 2000, 96(13), 4319-27; Hickey and Cotter Biol Chem 2006, 281(5), 2441-50); diffuse large B cell lymphoma (DLBCL) (Uddin, Hussain et al. Blood 2006, 108(13), 4178-86); several subtypes of sarcoma (Hernando, Charytonowicz et al. Nat Med 2007, 13(6), 748-53; Wan and Helman Oncologist 2007, 12(8), 1007-18); rhabdomyosarcoma (Cao, Yu et al. Cancer Res 2008, 68(19), 8039-8048; Wan, Shen et al. Neoplasia 2006, 8(5), 394-401); ovarian cancer (Shayesteh, Lu et al. Nat Genet, 1999, 21(1), 99-102; (Lee, Choi et al Gynecol Oncol 2005, 97(1) 26-34); endometrial tumors (Obata, Morland et al. Cancer Res 1998, 58(10), 2095-7; Lu, Wu et al. Clin Cancer Res 2008, 14(9), 2543-50); non small cell lung carcinoma (NSCLC) (Tang, He et al. Lung Cancer 2006, 51(2), 181-91; Marsit, Zheng et al. Hum Pathol 2005, 36(7), 768-76); small cell, squamous, large cell and adenocarcinoma (Massion, Taflan et al. Am J Respir Crit Care Med 2004, 170(10), 1088-94); lung tumors in general (Kokubo, Gemma et al. Br J Cancer 2005, 92(9), 1711-9; Pao, Wang et al. Pub Library of Science Med 2005, 2(1), e17); colorectal tumors (Velho, Oliveira et al. Eur J Cancer 2005, 41(11), 1649-54; Foukas, Claret et al. Nature, 2006, 441(7091), 366-370), particularly those that display microsatellite instability (Goel, Arnold et al. Cancer Res 2004, 64(9), 3014-21; Nassif, Lobo et al. Oncogene 2004, 23(2), 617-28), KRAS-mutated colorectal tumors (Bos Cancer Res 1989. 49(17), 4682-9; Fearon Ann N Y Acad Sci 1995, 768, 101-10); gastric carcinomas (Byun, Cho et al. Int J Cancer 2003, 104(3), 318-27); hepatocellular tumors (Lee, Soung et al. Oncogene 2005, 24(8), 1477-80); liver tumors (Hu, Huang et al. Cancer 2003, 97(8), 1929-40; Wan, Jiang et al. Cancer Res Clin Oncol 2003, 129(2), 100-6); primary melanomas and associated increased tumor thickness (Guldberg, thor Straten et al. Cancer Res 1997, 57(17), 3660-3; Tsao, Zhang et al. Cancer Res 2000, 60(7), 1800-4; Whiteman, Zhou et al. Int J Cancer 2002, 99(1), 63-7; Goel, Lazar et al. J Invest Dermatol 126(1), 2006, 154-60); pancreatic tumors (Asano, Yao et al. Oncogene 2004, 23(53), 8571-80); prostate carcinoma (Cairns, Okami et al. Cancer Res 1997, 57(22), 4997-5000; Gray, Stewart et al. Br J Cancer 1998, 78(10), 1296-300; Wang, Parsons et al. Clin Cancer Res 1998, 4(3), 811-5; Whang, Wu et al. Proc Natl Acad Sci USA 1998, 95(9), 5246-50; Majumder and Sellers Oncogene 2005, 24(50) 7465-74; Wang, Garcia et al. Proc Natl Acad Sci USA 2006, 103(5), 1480-5; (Lu, Ren et al. Int J Oncol 2006, 28(1), 245-51; Mulholland, Dedhar et al. Oncogene 25(3), 2006, 329-37; Xin, Teitell et al. Proc Natl Acad Sci US A 12006, 03(20), 7789-94; Mikhailova, Wang et al. Adv Exp Med Biol 2008, 617, 397-405; Wang, Mikhailova et al. Oncogene 2008, 27(56), 7106-7117); thyroid carcinoma, particularly in the anaplastic subtype (Garcia-Rostan, Costa et al. Cancer Res 2005, 65(22), 10199-207); follicular thyroid carcinoma (Wu, Mambo et al. J Clin Endocrinol Metab 2005, 90(8), 4688-93); anaplastic large cell lymphoma (ALCL); hamaratomas, angiomyelolipomas, TSC-associated and sporadic lymphangioleiomyomatosis: Cowden's disease (multiple hamaratoma syndrome) (Bissler, McCormack et al. N Engl J Med 2008, 358(2), 140-151); sclerosing hemangioma (Randa M. S. Amin Pathology International 2008, 58(1), 38-44); Peutz-Jeghers syndrome (PJS); head and neck cancer (Gupta, McKenna et al. Clin Cancer Res 2002, 8(3), 885-892); neurofibromatosis (Ferner Eur J Hum Genet 2006, 15(2), 131-138; Sabatini Nat Rev Cancer 2006, 6(9), 729-734; Johannessen, Johnson et al. Current Biology 2008, 18(1), 56-62); macular degeneration; macular edema; myeloid leukemia; systemic lupus; and autoimmune lymphoproliferative syndrome (ALPS).

### BRIEF DESCRIPTION OF THE DRAWINGS

FIG. 1 depicts Western Blots used to determine IC₅₀ of a PI3K-α selective compound measuring PI3K pathway inhibition in two distinct cell lines harboring two different genetic PI3K-α mutations.

FIG. 2A depicts a graph measuring PI3K pathway inhibition of a PI3K-α selective compound in PIK3CA H1047R models and in PIK3CA E545L models.

FIG. 2B depicts a graph measuring PI3K pathway inhibition of a dual PI3K-α/mTOR selective compound in PIK3CA H1047R models and in PIK3CA E545L models.

FIG. 2C depicts a graph measuring PI3K pathway inhibition of a PI3K-α selective compound in PIK3CA H1047R models and in PIK3CA E545L models.

FIG. 2D depicts a graph measuring PI3K pathway inhibition of a dual PI3K-α/mTOR selective compound in PIK3CA H1047R models and in PIK3CA E545L models.

FIG. 3A depicts a graph illustrating the effects of PI3K-β selective compound on the inhibition of PI3K pathway inhibition by a PI3K-α selective compound in a cell line harboring a PI3K-α mutation (E545K).

FIG. 3B depicts a graph illustrating the effects of PI3K-β selective compound on the inhibition of PI3K pathway inhibition by a PI3K-α selective compound in a cell line harboring a wild-type PI3K-α.

FIG. 3C depicts a graph illustrating the effects of PI3K-β selective compound on the inhibition of PI3K pathway inhibition by a PI3K-α selective compound in a cell line harboring a PI3K-α mutation (H1047R).

FIG. 4A depicts a bar chart representing the effect a PI3K-β selective compound has on PI3K pathway inhibition in various cell lines in the presence of a PI3K-α selective compound.

FIG. 4B depicts a bar chart representing the effect a PI3K-β selective compound has on PI3K pathway inhibition in various cell lines in the presence of a PI3K-α selective compound.

FIG. 4C depicts a bar chart representing the effect a PI3K-β selective compound has on PI3K pathway inhibition in various cell lines in the presence of a PI3K-α selective compound.

FIG. 4D depicts a bar chart representing the effect a PI3K-β selective compound has on PI3K pathway inhibition in various cell lines in the presence of a pan PI3K inhibitor compound.

### SUMMARY OF THE INVENTION

The following only summarizes certain aspects of the invention and is not intended to be limiting in nature. These aspects and other aspects and embodiments are described more fully below. All references cited in this specification are hereby incorporated by reference in their entirety. In the event of a discrepancy between the express disclosure of this specification and the references incorporated by reference, the express disclosure of this specification shall control.

We recognized the important role of PI3K and mTOR in biological processes and disease states and, therefore, realized that inhibitors of these protein kinases would be desirable. Accordingly, the invention provides compounds that inhibit, regulate, and/or modulate PI3K and/or mTOR that are useful in the treatment of hyperproliferative diseases, such as cancer, in mammals. This invention also provides methods of making the compound, methods of using such compounds in the treatment of hyperproliferative diseases in mammals, especially humans, and to pharmaceutical compositions containing such compounds.

The important role of PI3K-α and mTOR in biological processes and disease states was recognized and, therefore, realized that inhibitors of these protein kinases would be desirable. Accordingly, the invention provides treatment methods, methods for selectively screening compounds that are selective towards cancers that are mediated by specific genetic lesions in PI3CA and methods for identifying a treatment regimen for patients with cancer. In other aspects, the present invention provides compounds that inhibit, regulate, and/or modulate PI3K-α and/or mTOR and are useful in the treatment of hyperproliferative diseases, such as cancer, in mammals, for example, humans.

A first aspect of the invention provides a compound of Formula I:

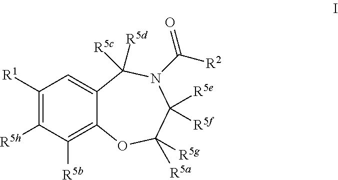

or a single stereoisomer or mixture of isomers thereof and additionally optionally as a pharmaceutically acceptable salt thereof, where
R¹ is phenyl optionally substituted with one, two, or three R⁶ groups; or
R¹ is heteroaryl optionally substituted with one, two, or three R⁷;
R² is —NR³R⁴;
R³ is hydrogen, alkyl, or alkoxycarbonylalkyl; and R⁴ is optionally substituted cycloalkyl, optionally substituted phenyl, optionally substituted phenylalkyl, optionally substituted heteroaryl, or optionally substituted heteroarylalkyl; or
R³ and R⁴ together with the nitrogen to which they are attached form HET optionally substituted on any substitutable atom of the ring with R¹⁰, R^{10a}, R^{10b}, R^{10c}, R^{10d}, R^{10e}, and R^{10f};
HET is
   i. a saturated or partially unsaturated, but non-aromatic, monocyclic 5- to 8-membered ring optionally containing an additional one or two ring heteroatoms which are independently oxygen, sulfur, or nitrogen where the remaining ring atoms are carbon; or
   ii. a partially unsaturated, but not aromatic, monocyclic 5- to 8-membered ring optionally containing an additional one or two ring heteroatoms which are independently oxygen, sulfur, or nitrogen and the remaining ring atoms are carbon and which ring is fused to a benzo ring; or
   iii. a fused, bridged, or spirocyclic, bicyclic 7- to 11-membered ring optionally containing an additional one or two heteroatoms which are independently oxygen, sulfur, or nitrogen and the remaining ring atoms are carbon and where each ring of the 7- to 1-membered ring is saturated or partially unsaturated but not fully aromatic; or
   iv. a fused, bridged, or spirocyclic, bicyclic 7- to 11-membered ring optionally containing an additional one or two ring heteroatoms which are independently oxygen, sulfur, or nitrogen and the remaining ring atoms are carbon where each ring of the bicyclic 7- to 11-membered ring is saturated or partially unsaturated but not fully aromatic, and where the bicyclic 7- to 11-membered ring is fused to a benzo ring;
R^{5a} and R^{5c} are independently hydrogen or alkyl;
R^{5h} is hydrogen or halo;
R^{5b} is hydrogen, amino, or halo;
R^{5d}, R^{5e}, R^{5f}, and R^{5g} are hydrogen;
each R⁶, when R⁶ is present, is independently nitro; cyano; halo; alkyl; alkenyl; alkynyl; halo; haloalkyl; —OR^{8a}; —NR⁸R^{8a}; —C(O)NR⁸R^{8a}; —NR⁸C(O)OR⁹; —NR⁸C(O)R⁹; —NR⁸S(O)₂R^{8a}; —NR⁸C(O)NR^{8a}R⁹; carboxy, —C(O)OR⁹; alkylcarbonyl; alkyl substituted with one or two —C(O)NR⁸R^{8a}; heteroaryl optionally substituted with 1, 2, or 3 R¹⁴; or optionally substituted heterocycloalkyl;
each R⁷, when R⁷ is present, is independently oxo; nitro; cyano; alkyl; alkenyl; alkynyl; halo; haloalkyl; hydroxyalkyl; alkoxyalkyl; —OR^{8a}; —SR¹³; —S(O)R¹³; —S(O)₂R¹³; —NR⁸R^{8a}; —C(O)NR⁸R^{8a}; —NR⁸C(O)OR⁹; —NR⁸C(O)R⁹; —NR⁸S(O)₂R^{8a}; —NR⁸C(O)NR^{8a}R⁹; carboxy; —C(O)OR⁹; alkylcarbonyl; —S(O)₂NR⁸R⁹; alkyl substituted with one or two —NR⁸R^{8a}; alkyl substituted with one or two —NR C(O)R^{8a}; optionally substituted cycloalkyl; optionally substituted cycloalkylalkyl; optionally substituted heterocycloalkyl; optionally substituted heterocycloalkylalkyl; optionally substituted heteroaryl; or optionally substituted heteroarylalkyl;
R⁸ is hydrogen, alkyl, alkenyl, alkynyl, hydroxyalkyl, or haloalkyl;
R^{8a} is hydrogen, alkyl, alkenyl, alkynyl, haloalkyl, hydroxyalkyl, cyanoalkyl, aminoalkyl, alkylaminoalkyl, dialkylaminoalkyl, alkoxyalkyl, optionally substituted cycloalkyl, optionally substituted cycloalkylalkyl, optionally substituted heterocycloalkyl, optionally substituted heterocycloalkylalkyl, optionally substituted phenyl, optionally substituted phenylalkyl, optionally substituted heteroaryl, or optionally substituted heteroarylalkyl;
R⁹ is alkyl, alkenyl, alkynyl, hydroxyalkyl, alkoxyalkyl, haloalkyl, or optionally substituted heterocycloalkylalkyl;
R¹⁰, R^{10a}, R^{10b}, R^{10c}, R^{10d}, R^{10e}, and R^{10f} are independently hydrogen; halo; alkyl; haloalkyl; haloalkenyl; hydroxyalkyl; alkylthio; alkylsulfonyl; hydroxy; alkoxy; haloalkoxy; cyano; alkoxycarbonyl; carboxy; amino; alkylamino; dialkylamino; —C(O)R¹²; —C(O)NR¹¹R^{11a}; optionally substituted cycloalkyl; optionally substituted cycloalkylalkyl; optionally substituted phenyl; optionally substituted phenylalkyl; optionally substituted phenyloxy; optionally substituted phenyloxyalkyl; optionally substituted heterocycloalkyl; optionally substituted heterocycloalkylalkyl; optionally substituted heteroaryl; or optionally substituted heteroarylalkyl; or two of R¹⁰, R^{10a}, R^{10b}, R^{10c}, R^{10d}, R^{10e}, and R^{10f} when attached to the same carbon form oxo, imino, or thiono;
R¹¹ hydrogen, alkyl, or alkenyl;
R^{11a} hydrogen, alkyl, or alkenyl;
R¹² is alkyl, or optionally substituted heteroaryl;
R¹³ is alkyl or haloalkyl; and
each R¹⁴, when R¹⁴ is present, is independently amino, alkylamino, dialkylamino, acylamino, halo, hydroxy, alkyl, haloalkyl, hydroxyalkyl, aminoalkyl, alkylaminoalkyl, dialkylaminoalkyl, alkoxycarbonyl, aminocarbonyl, alkylaminocarbonyl, dialkylaminocarbonyl, or optionally substituted phenyl.

In a second aspect, the invention is directed to a pharmaceutical composition which comprises 1) a compound of Formula I or a single stereoisomer or mixture of isomers thereof, optionally as a pharmaceutically acceptable salt thereof and 2) a pharmaceutically acceptable carrier, excipient, or diluent.

In a third aspect of the invention is a method of inhibiting the in vivo activity of PI3K and additionally optionally mTOR, the method comprising administering to a patient an effective PI3K-inhibiting and additionally optionally mTOR-inhibiting amount of a Compound of Formula Ia Compound of Formula I or a single stereoisomer or mixture of stereoisomers thereof, optionally as a pharmaceutically acceptable salt or solvate thereof or pharmaceutical composition thereof.

In a fourth aspect, the Invention provides a method for treating a disease, disorder, or syndrome which method comprises administering to a patient a therapeutically effective amount of a compound of Formula I or a single stereoisomer or mixture of isomers thereof, optionally as a pharmaceutically acceptable salt or solvate thereof, or a pharmaceutical composition comprising a therapeutically effective amount of a compound of Formula I or a single stereoisomer or mixture of isomers thereof, optionally as a pharmaceutically acceptable salt or solvate thereof, and a pharmaceutically acceptable carrier, excipient, or diluent.

In a fifth aspect, the Invention provides a method for making a Compound of Formula I(a) which method comprises

reacting the following intermediate, or a salt thereof:

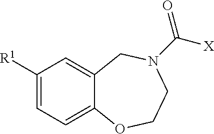

where X is halo and R¹ is as defined in the Summary of the Invention for a Compound of Formula I; with an intermediate of formula R²H where R² is as defined in the Summary of the Invention for a Compound of Formula I to yield a Compound of the Invention of Formula I(a)

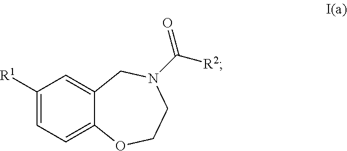

and optionally separating individual isomers; and optionally modifying any of the R¹ and R² groups; and optionally forming a pharmaceutically acceptable salt thereof; or

reacting the following intermediate, or a salt thereof:

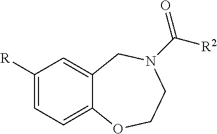

where R is halo or —B(OR′)₂ (where both R′ are hydrogen or the two R′ together form a boronic ester), and R² is as defined in the Summary of the Invention for a Compound of Formula I; with an intermediate of formula R¹Y where Y is halo when R is —B(OR)₂ and Y is —B(OR)₂ when R is halo, and R² is as defined in the Summary of the Invention for a Compound of Formula I to yield a Compound of the Invention of Formula I(a); and optionally separating individual isomers; and optionally modifying any of the R¹ and R² groups; and optionally forming a pharmaceutically acceptable salt, hydrate, solvate or combination thereof.

In a sixth aspect of the invention provides a method for treating a subject having a tumor the method comprising: (a) administering a PI3K-α selective inhibitor, a dual PI3K-α/mTOR selective inhibitor, or a combination of a PI3K-α selective inhibitor and a mTOR selective inhibitor to the subject if the tumor comprises a mutation in a PI3K-α kinase domain; or (b) administering a combination of a PI3K-α selective inhibitor and a PI3K-β selective inhibitor, a dual PI3K-α/mTOR selective inhibitor, or a PI3K-β selective inhibitor, to the subject if the tumor comprises a mutation in a PI3K-α helical domain.

In a seventh aspect, the present invention provides a method for identifying a selective inhibitor of a PI3K isozyme, the method comprising: (a) contacting a first cell bearing a first mutation in a PI3K-α with a candidate inhibitor; (b) contacting a second cell bearing a wild type PI3K-α, a PTEN null mutation, or a second mutation in the PI3K-α with the candidate inhibitor; and (c) measuring AKT phosphorylation in said first and said second cells, wherein decreased AKT phosphorylation in said first cell when compared to said second cell identifies said candidate inhibitor as a selective PI3K-α inhibitor.

In an eighth another aspect, the present invention provides for a method for determining a treatment regimen for a cancer patient having a tumor comprising a PI3K-α, the method comprising: determining the presence or absence of a mutation in amino acids 1047 and/or 545 of said PI3K-α; wherein if said PI3K-α has a mutation at position 1047, said method comprises administering to the cancer patient a therapeutically effective amount of a PI3K-α selective inhibitor compound, or a dual PI3K-α/mTOR selective inhibitor, or a combination of a PI3K-α selective inhibitor and a mTOR selective inhibitor; or wherein if said PI3K-α has a mutation at position 545, said method comprises administering to the cancer patient a therapeutically effective amount of a combination of a PI3K-α selective inhibitor and a PI3K-β selective inhibitor, or a dual PI3K-α/mTOR selective inhibitor, or a combination of a PI3K-α selective inhibitor and a mTOR selective inhibitor.

In a ninth aspect, the cell used to diagnose, treat or screen against includes a cancer or tumor cell obtained from a tumor or cancer derived from: a breast cancer, a mantle cell lymphoma, mantle cell lymphoma, a renal cell carcinoma, an acute myelogenous leukemia, a chronic myelogenous leukemia, a NPM/ALK-transformed anaplastic large cell lymphoma, a diffuse large B cell lymphoma, a rhabdomyosarcoma, an ovarian cancer, an endometrial cancer, a cervical cancer, a non-small cell lung carcinoma, a small-cell lung carcinoma, a melanoma, a pancreatic cancer, a prostate carcinoma, a thyroid carcinoma, an anaplastic large cell lymphoma, a hemangioma, a glioblastoma, or a head and neck cancer.

### DETAILED DESCRIPTION OF THE INVENTION

The present disclosure incorporates the International Patent Application No. PCT/US2010/035565, filed May, 20, 2010, by reference herein in its entirety.

### ABBREVIATIONS AND DEFINITIONS

The following abbreviations and terms have the indicated meanings throughout:

| Abbreviation | Meaning |
|---|---|
| br | broad |
| ° C. | degrees Celsius |
| d | doublet |
| dd | doublet of doublet |
| dt | doublet of triplet |
| DCM | dichloromethane |
| DIEA or DIPEA | N,N-di-isopropyl-N-ethylamine |
| DMA | N,N-dimethylacetamide |
| DME | 1,2-dimethoxyethane |
| DMF | N,N-dimethylformamide |
| DMSO | dimethyl sulfoxide |
| dppf | 1,1′-bis(diphenylphosphano)ferrocene |
| EI | Electron Impact ionization |
| g | gram(s) |
| GC/MS | gas chromatography/mass spectrometry |
| h or hr | hour(s) |
| HPLC | high pressure liquid chromatography |
| L | liter(s) |
| LC/MS | liquid chromatography/mass spectrometry |
| M | molar or molarity |
| m | Multiplet |
| MeOH | methanol |
| mg | milligram(s) |
| MHz | megahertz (frequency) |
| min | minute(s) |
| mL | milliliter(s) |
| μL | microliter(s) |
| μM | micromolar |
| μmol | micromole(s) |
| mM | Millimolar |
| mmol | millimole(s) |
| mol | mole(s) |
| MS | mass spectral analysis |
| N | normal or normality |
| nM | nanomolar |
| NMP | N-methyl-2-pyrrolidone |
| NMR | nuclear magnetic resonance spectroscopy |
| q | Quartet |
| rt | Room temperature |
| s | Singlet |
| t or tr | Triplet |
| THF | tetrahydrofuran |

The symbol “—” means a single bond, “ [symbol] ” means a double bond, “ [symbol] ” means a triple bond, “ [symbol] ” means a single or double bond. The symbol “ [symbol] ” refers to a group on a double-bond as occupying either position on the terminus of a double bond to which the symbol is attached; that is, the geometry, E- or Z-, of the double bond is ambiguous. When a group is depicted removed from its parent Formula, the “ [symbol] ” symbol will be used at the end of the bond which was theoretically cleaved in order to separate the group from its parent structural Formula.

When chemical structures are depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes a particular atom in a structure is described in textual Formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

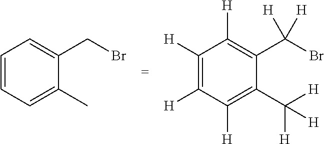

If a group “R” is depicted as “floating” on a ring system, as for example in the Formula:

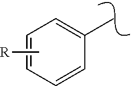

then, unless otherwise defined, a substituent “R” may reside on any atom of the ring system, assuming replacement of a depicted, implied, or expressly defined hydrogen from one of the ring atoms, so long as a stable structure is formed.

If a group “R” is depicted as floating on a fused or bridged ring system, as for example in the Formula e:

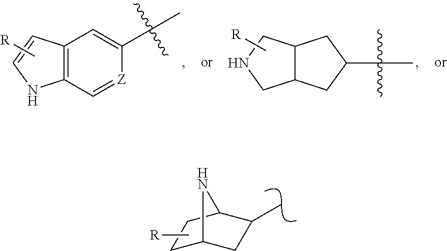

then, unless otherwise defined, a substituent “R” may reside on any atom of the fused or bridged ring system, assuming replacement of a depicted hydrogen (for example the —NH— in the Formula above), implied hydrogen (for example as in the Formula above, where the hydrogens are not shown but understood to be present), or expressly defined hydrogen (for example where in the Formula above, “Z” equals ═CH—) from one of the ring atoms, so long as a stable structure is formed. In the example depicted, the “R” group may reside on either the 5-membered or the 6-membered ring of the fused or bridged ring system.

When a group “R” is depicted as existing on a ring system containing saturated carbons, as for example in the Formula:

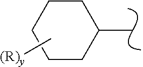

where, in this example, “y” can be more than one, assuming each replaces a currently depicted, implied, or expressly defined hydrogen on the ring; then, unless otherwise defined, where the resulting structure is stable, two “R's” may reside on the same carbon. In another example, two R's on the same carbon, including that carbon, may form a ring, thus creating a spirocyclic ring structure with the depicted ring as for example in the Formula:

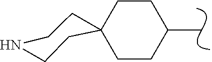

“Acyl” means a —C(O)R radical where R is alkyl, haloalkyl, alkenyl, cycloalkyl, cycloalkylalkyl, aryl, aralkyl, heteroaryl, heteroaralkyl, heterocycloalkyl, or heterocycloalkylalkyl, as defined herein, e.g., acetyl, trifluoromethylcarbonyl, or 2-methoxyethylcarbonyl, and the like.

“Acylamino” means a —NRR′ radical where R is hydrogen, hydroxy, alkyl, or alkoxy and R′ is acyl, as defined herein.

“Acyloxy” means an —OR radical where R is acyl, as defined herein, e.g. cyanomethylcarbonyloxy, and the like.

“Administration” and variants thereof (e.g., “administering” a compound) in reference to a compound of the invention means introducing the compound of the compound into the system of the animal in need of treatment. When a compound of the invention or prodrug thereof is provided in combination with one or more other active agents (e.g., surgery, radiation, and chemotherapy, etc.), “administration” and its variants are each understood to include concurrent and sequential introduction of the compound or prodrug thereof and other agents.

“Alkenyl” means a means a linear monovalent hydrocarbon radical of two to six carbon atoms or a branched monovalent hydrocarbon radical of three to 6 carbon atoms which radical contains at least one double bond, e.g., ethenyl, propenyl, 1-but-3-enyl, and 1-pent-3-enyl, and the like.

“Alkoxy” means an —OR group where R is alkyl group as defined herein. Examples include methoxy, ethoxy, propoxy, isopropoxy, and the like.

“Alkoxyalkyl” means an alkyl group, as defined herein, substituted with at least one, specifically one, two, or three, alkoxy groups as defined herein. Representative examples include methoxymethyl and the like.

“Alkoxycarbonyl” means a —C(O)R group where R is alkoxy, as defined herein.

“Alkyl” means a linear saturated monovalent hydrocarbon radical of one to six carbon atoms or a branched saturated monovalent hydrocarbon radical of three to 6 carbon atoms, e.g., methyl, ethyl, propyl, 2-propyl, butyl (including all isomeric forms), or pentyl (including all isomeric forms), and the like.

“Alkylamino” means an —NHR group where R is alkyl, as defined herein.

“Alkylaminoalkyl” means an alkyl group substituted with one or two alkylamino groups, as defined herein.

“Alkylaminoalkyloxy” means an —OR group where R is alkylaminoalkyl, as defined herein.

“Alkylcarbonyl” means a —C(O)R group where R is alkyl, as defined herein.

“Alkylsulfonyl” means an —S(O)₂R group where R is alkyl, as defined herein.

“Alkylsulfonylalkyl” means an alkyl group, as defined herein, substituted with at least one, preferably one or two, alkylsulfonyl groups, as defined herein.

“Alkynyl” means a linear monovalent hydrocarbon radical of two to six carbon atoms or a branched monovalent hydrocarbon radical of three to 6 carbon atoms which radical contains at least one triple bond, e.g., ethynyl, propynyl, butynyl, pentyn-2-yl and the like.

“Amino” means —NH₂.

“Aminoalkyl” means an alkyl group substituted with at least one, specifically one, two or three, amino groups.

“Aminoalkyloxy” means an —OR group where R is aminoalkyl, as defined herein.

“Aminocarbonyl” means a —C(O)NH₂ group.

“Alkylaminocarbonyl” means a —C(O)NHR group where R is alkyl as defined herein.

“Aryl” means a monovalent six- to fourteen-membered, mono- or bi-carbocyclic ring, wherein the monocyclic ring is aromatic and at least one of the rings in the bicyclic ring is aromatic. Unless stated otherwise, the valency of the group may be located on any atom of any ring within the radical, valency rules permitting. Representative examples include phenyl, naphthyl, and indanyl, and the like.

“Arylalkyl” means an alkyl radical, as defined herein, substituted with one or two aryl groups, as defined herein, e.g., benzyl and phenethyl, and the like.

“Arylalkyloxy” means an —OR group where R is arylakyl, as defined herein.

“Cancer” refers to cellular-proliferative disease states, including but not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hanlartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiforme, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, SertoliLeydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell”, a “tumor” or” tumor cell” as provided herein, includes a cell or group of cells afflicted by any one of the above-identified conditions.

“Cyanoalkyl” means an alkyl group, as defined herein, substituted with one or two cyano groups.

“Cycloalkyl” means a monocyclic or fused or bridged bicyclic or tricyclic, saturated or partially unsaturated (but not aromatic), monovalent hydrocarbon radical of three to ten carbon ring atoms. Unless stated otherwise, the valency of the group may be located on any atom of any ring within the radical, valency rules permitting. One or two ring carbon atoms may be replaced by a —C(O)—, —C(S)—, or —C(═NH)— group. More specifically, the term cycloalkyl includes, but is not limited to, cyclopropyl, cyclobutyl, cyclopentyl, cyclohexyl, cyclohexyl, cyclohex-3-enyl, or (1r,3r,5R,7R)-tricyclo[3.3.1.1^{3,7}]decan-2-yl, and the like.

“Cycloalkylalkyl” means an alkyl group substituted with at least one, specifically one or two, cycloalkyl group(s) as defined herein.

“Dialkylamino” means a —NRR′ radical where R and R′ are alkyl as defined herein, or an N-oxide derivative, or a protected derivative thereof, e.g., dimethylamino, diethylamino, N,N-methylpropylamino or N,N-methylethylamino, and the like.

“Dialkylaminoalkyl” means an alkyl group substituted with one or two dialkylamino groups, as defined herein.

“Dialkylaminoalkyloxy” means an —OR group where R is dialkylaminoalkyl, as defined herein. Representative examples include 2-(N,N-diethylamino)-ethyloxy, and the like.

“Dialkylaminocarbonyl” means a —C(O)NRR′ group where R and R′ are alkyl as defined herein.

“Halogen” or “halo” refers to fluorine, chlorine, bromine and iodine.

“Haloalkoxy” means an —OR′ group where R′ is haloalkyl as defined herein, e.g., trifluoromethoxy or 2,2,2-trifluoroethoxy, and the like.

“Haloalkyl” mean an alkyl group substituted with one or more halogens, specifically 1, 2, 3, 4, 5, or 6 halo atoms, e.g., trifluoromethyl, 2-chloroethyl, and 2,2-difluoroethyl, and the like.

“Heteroaryl” means a monocyclic or fused or bridged bicyclic monovalent radical of 5 to 14 ring atoms containing one or more, specifically one, two, three, or four ring heteroatoms where each heteroatom is independently —O—, —S(O)ₙ— (n is 0, 1, or 2), —NH—, —N═, or N-oxide, with the remaining ring atoms being carbon, wherein the ring comprising a monocyclic radical is aromatic and wherein at least one of the fused rings comprising the bicyclic radical is aromatic. One or two ring carbon atoms of any nonaromatic rings comprising a bicyclic radical may be replaced by a —C(O)—, —C(S)—, or —C(═NH)— group. Unless stated otherwise, the valency may be located on any atom of any ring of the heteroaryl group, valency rules permitting. More specifically, the term heteroaryl includes, but is not limited to, 1,2,4-triazolyl, 1,3,5-triazolyl, phthalimidyl, pyridinyl, pyrrolyl, imidazolyl, thienyl, furanyl, indolyl, 2,3-dihydro-1H-indolyl (including, for example, 2,3-dihydro-1H-indol-2-yl or 2,3-dihydro-1H-indol-5-yl, and the like), isoindolyl, indolinyl, isoindolinyl, benzimidazolyl, benzodioxol-4-yl, benzofuranyl, cinnolinyl, indolizinyl, naphthyridin-3-yl, phthalazin-3-yl, phthalazin-4-yl, pteridinyl, purinyl, quinazolinyl, quinoxalinyl, tetrazoyl, pyrazolyl, pyrazinyl, pyrimidinyl, pyridazinyl, oxazolyl, isooxazolyl, oxadiazolyl, benzoxazolyl, quinolinyl, isoquinolinyl, tetrahydroisoquinolinyl (including, for example, tetrahydroisoquinolin-4-yl or tetrahydroisoquinolin-6-yl, and the like), pyrrolo[3,2-c]pyridinyl (including, for example, pyrrolo[3,2-c]pyridin-2-yl or pyrrolo[3,2-c]pyridin-7-yl, and the like), benzopyranyl, 2,3-dihydrobenzofuranyl, benzo[d][1,3]dioxolyl, 2,3-dihydrobenzo[b][1,4]dioxinyl, thiazolyl, isothiazolyl, thiadiazolyl, benzothiazolyl, benzothienyl, and the derivatives thereof, or N-oxide or a protected derivative thereof. The term “5- or 6-membered heteroaryl” describes a subset of the term “heteroaryl.”

“Heteroarylalkyl” means an alkyl group, as defined herein, substituted with at least one, specifically one or two heteroaryl group(s), as defined herein.

“Heterocycloalkyl” means a saturated or partially unsaturated (but not aromatic) monovalent monocyclic group of 3 to 8 ring atoms or a saturated or partially unsaturated (but not aromatic) monovalent fused or bridged, bicyclic or tricyclic group of 5 to 12 ring atoms in which one or more, specifically one, two, three, or four ring heteroatoms where each heteroatom is independently O, S(O)ₙ (n is 0, 1, or 2), —N═, or —NH—, the remaining ring atoms being carbon. One or two ring carbon atoms may be replaced by a —C(O)—, —C(S)—, or —C(═NH)— group. Unless otherwise stated, the valency of the group may be located on any atom of any ring within the radical, valency rules permitting. When the point of valency is located on a nitrogen atom, R^{y} is absent. More specifically the term heterocycloalkyl includes, but is not limited to, azetidinyl, pyrrolidinyl, 2-oxopyrrolidinyl, 2,5-dihydro-1H-pyrrolyl, piperidinyl, 4-piperidonyl, morpholinyl, piperazinyl, 2-oxopiperazinyl, tetrahydropyranyl, 2-oxopiperidinyl, thiomorpholinyl, thiamorpholinyl, perhydroazepinyl, pyrazolidinyl, imidazolinyl, imidazolidinyl, dihydropyridinyl, tetrahydropyridinyl, oxazolinyl, oxazolidinyl, isoxazolidinyl, thiazolinyl, thiazolidinyl, quinuclidinyl, isothiazolidinyl, octahydrocyclopenta[c]pyrrolyl, octahydroindolyl, octahydroisoindolyl, decahydroisoquinolyl, tetrahydrofuryl, tetrahydropyranyl, (3aR,6aS)-5-methyloctahydrocyclopenta[c]pyrrolyl, and (3aS,6aR)-5-methyl-1,2,3,3a,4,6a-hexahydrocyclopenta[c]pyrrolyl, and the derivatives thereof and N-oxide or a protected derivative thereof.

“Heterocycloalkylalkyl” means an alkyl radical, as defined herein, substituted with one or two heterocycloalkyl groups, as defined herein, e.g., morpholinylmethyl, N-pyrrolidinylethyl, and 3-(N-azetidinyl)propyl, and the like.

“Heterocycloalkyloxy” means an —OR group where R is heterocycloalkyl, as defined herein.

“Hydroxyalkyl” means an alkyl group, as defined herein, substituted with at least one, preferably 1, 2, 3, or 4, hydroxy groups.

“Phenylalkyl” means an alkyl group, as defined herein, substituted with one or two phenyl groups.

“Phenylalkyloxy” means an —OR group where R is phenylalkyl, as defined herein.

“Optional” or “optionally” means that the subsequently described event or circumstance may or may not occur, and that the description includes instances where said event or circumstance occurs and instances in which it does not. One of ordinary skill in the art would understand that with respect to any molecule described as containing one or more optional substituents, only sterically practical and/or synthetically feasible compounds are meant to be included. “Optionally substituted” refers to all subsequent modifiers in a term, unless stated otherwise. A list of exemplary optional substitutions is presented below in the definition of “substituted.”

“Optionally substituted aryl” means an aryl group, as defined herein, optionally substituted with one, two, or three substituents independently acyl, acylamino, acyloxy, alkyl, haloalkyl, alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, or aminoalkoxy; or aryl is pentafluorophenyl. Within the optional substituents on “aryl”, the alkyl and alkenyl, either alone or as part of another group (including, for example, the alkyl in alkoxycarbonyl), are independently optionally substituted with one, two, three, four, or five halo.

“Optionally substituted arylalkyl” means an alkyl group, as defined herein, substituted with optionally substituted aryl, as defined herein.

“Optionally substituted cycloalkyl” means a cycloalkyl group, as defined herein, substituted with one, two, or three groups independently acyl, acyloxy, acylamino, alkyl, haloalkyl, alkenyl, alkoxy, alkenyloxy, alkoxycarbonyl, alkenyloxycarbonyl, alkylthio, alkylsulfinyl, alkylsulfonyl, aminosulfonyl, alkylaminosulfonyl, dialkylaminosulfonyl, alkylsulfonylamino, halo, hydroxy, amino, alkylamino, dialkylamino, aminocarbonyl, alkylaminocarbonyl, dialkylaminocarbonyl, nitro, alkoxyalkyloxy, aminoalkoxy, alkylaminoalkoxy, dialkylaminoalkoxy, carboxy, or cyano. Within the above optional substituents on “cycloalkyl”, the alkyl and alkenyl, either alone or as part of another substituent on the cycloalkyl ring, are independently optionally substituted with one, two, three, four, or five halo, e.g. haloalkyl, haloalkoxy, haloalkenyloxy, or haloalkylsulfonyl.

“Optionally substituted cycloalkylalkyl” means an alkyl group substituted with at least one, specifically one or two, optionally substituted cycloalkyl groups, as defined herein.

“Optionally substituted heteroaryl” means a heteroaryl group optionally substituted with one, two, or three substituents independently acyl, acylamino, acyloxy, alkyl, haloalkyl, alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, aminoalkoxy, alkylaminoalkoxy, or dialkylaminoalkoxy. Within the optional substituents on “heteroaryl”, the alkyl and alkenyl, either alone or as part of another group (including, for example, the alkyl in alkoxycarbonyl), are independently optionally substituted with one, two, three, four, or five halo.

“Optionally substituted heteroarylalkyl” means an alkyl group, as defined herein, substituted with at least one, specifically one or two, optionally substituted heteroaryl group(s), as defined herein.

“Optionally substituted heterocycloalkyl” means a heterocycloalkyl group, as defined herein, optionally substituted with one, two, or three substituents independently acyl, acylamino, acyloxy, haloalkyl, alkyl, alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, aminoalkoxy, or phenylalkyl. Within the optional substituents on “heterocycloalkyl”, the alkyl and alkenyl, either alone or as part of another group (including, for example, the alkyl in alkoxycarbonyl), are independently optionally substituted with one, two, three, four, or five halo.

“Optionally substituted heterocycloalkylalkyl” means an alkyl group, as defined herein, substituted with at least one, specifically one or two, optionally substituted heterocycloalkyl group(s) as defined herein.

“Optionally substituted phenyl” means a phenyl group optionally substituted with one, two, or three substituents independently acyl, acylamino, acyloxy, alkyl, haloalkyl, alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, or aminoalkoxy, or aryl is pentafluorophenyl. Within the optional substituents on “phenyl”, the alkyl and alkenyl, either alone or as part of another group (including, for example, the alkyl in alkoxycarbonyl), are independently optionally substituted with one, two, three, four, or five halo.

“Optionally substituted phenylalkyl” means an alkyl group, as defined herein, substituted with one or two optionally substituted phenyl groups, as defined herein.

“Optionally substituted phenylsulfonyl” means an —S(O)₂R group where R is optionally substituted phenyl, as defined herein.

“Oxo” means an oxygen which is attached via a double bond.

“Yield” for each of the reactions described herein is expressed as a percentage of the theoretical yield.

“Metabolite” refers to the break-down or end product of a compound or its salt produced by metabolism or biotransformation in the animal or human body; for example, biotransformation to a more polar molecule such as by oxidation, reduction, or hydrolysis, or to a conjugate (see Goodman and Gilman, “The Pharmacological Basis of Therapeutics” 8.sup.th Ed., Pergamon Press, Gilman et al. (eds), 1990 for a discussion of biotransformation). As used herein, the metabolite of a compound of the invention or its salt may be the biologically active form of the compound in the body. In one example, a prodrug may be used such that the biologically active form, a metabolite, is released in vivo. In another example, a biologically active metabolite is discovered serendipitously, that is, no prodrug design per se was undertaken. An assay for activity of a metabolite of a compound of the present invention is known to one of skill in the art in light of the present disclosure.

“Oligonucleotide” or “oligonucleotide probes” or “polynucleotide” or “nucleotide” or “nucleic acid” refer to a biological polymer molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof.

“Oligonucleotide having a nucleotide sequence encoding a gene” or “a nucleic acid sequence encoding” a specified polypeptide refer to a nucleic acid sequence comprising the coding region of a gene or in other words the nucleic acid sequence which encodes a gene product. The coding region may be present in either a cDNA, genomic DNA or RNA form. When present in a DNA form, the oligonucleotide may be single-stranded (i.e., the sense strand) or double-stranded. Suitable expression control sequences or elements such as enhancers/promoters, splice junctions, polyadenylation signals, etc, may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

“Patient” and “Subject” are used interchangeably herein and for the purposes of the present invention includes humans and other animals, particularly mammals, and other organisms. Thus the methods are applicable to both human therapy and veterinary applications. In a specific embodiment the patient is a mammal, and in a more specific embodiment the patient is human.

A “pharmaceutically acceptable salt” of a compound means a salt that is pharmaceutically acceptable and that possesses the desired pharmacological activity of the parent compound. It is understood that the pharmaceutically acceptable salts are non-toxic. Additional information on suitable pharmaceutically acceptable salts can be found in Remington's Pharmaceutical Sciences, 17^{th} ed., Mack Publishing Company, Easton, Pa., 1985, which is incorporated herein by reference or S. M. Berge, et al., “Pharmaceutical Salts,” J. Pharm. Sci., 1977; 66:1-19 both of which are incorporated herein by reference.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

Examples of a pharmaceutically acceptable base addition salts include those formed when an acidic proton present in the parent compound is replaced by a metal ion, such as sodium, potassium, lithium, ammonium, calcium, magnesium, iron, zinc, copper, manganese, aluminum salts and the like. Specific salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins. Examples of organic bases include isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, tromethamine, N-methylglucamine, polyamine resins, and the like. Exemplary organic bases are isopropylamine, diethylamine, ethanolamine, trimethylamine, dicyclohexylamine, choline, and caffeine. “Platin(s),” and “platin-containing agent(s)” include, for example, cisplatin, carboplatin, and oxaliplatin.

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to their knowledge and to this disclosure.

“Preventing” or “prevention” of a disease, disorder, or syndrome includes inhibiting the disease from occurring in a human, i.e. causing the clinical symptoms of the disease, disorder, or syndrome not to develop in an animal that may be exposed to or predisposed to the disease, disorder, or syndrome but does not yet experience or display symptoms of the disease, disorder, or syndrome.

“Treating” or “treatment” of a disease, disorder, or syndrome, as used herein, includes (i) inhibiting the disease, disorder, or syndrome, i.e., arresting its development; and (ii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

Generally, the procedures for nucleic acid manipulations, e.g. cloning, amplification, hybridization, transfection, other molecular biology methods and the like, and cell cultures are common methods used in the art. Such standard techniques can be found in reference manuals such as for example, Sambrook et al. (1989, Molecular Cloning—A Laboratory Manual, Cold Spring Harbor. Laboratories), Herdewijn, ed., Oligonucleotide Synthesis: Methods and Applications (Methods in Molecular Biology), Humana Press, Totowa, N.J., 2004. and Ausubel et al. (1994, Current Protocols in Molecular Biology, Wiley, New York), all these references are incorporated by reference herein in their entireties.

Generally, procedures for production and use of antibodies, for example, immunoprecipitation, ELISA, and other uses of antibodies and related immunology methods and the like are common methods used in the art. Such standard techniques can be found in reference manuals such as for example, Kohler & Milstein (1975) Nature 256:495-497; Kozbor, et al. (1983) Immunology Today 4:72; Cole, et al., pp. 77-96 in Monoclonal Antibodies and Cancer Therapy (1985); Coligan (1991) Current Protocols in Immunology; Harlow & Lane (1988) Antibodies: A Laboratory Manual; and Goding (1986) Monoclonal Antibodies: Principles and Practice (2d ed.) all of these documents are incorporated herein in their entireties.

The compounds disclosed herein also include all pharmaceutically acceptable isotopic variations, in which at least one atom is replaced by an atom having the same atomic number, but an atomic mass different from the atomic mass usually found in nature. Examples of isotopes suitable for inclusion in the disclosed compounds include, without limitation, isotopes of hydrogen, such as ²H and ³H; isotopes of carbon, such as ¹³C and ¹⁴C; isotopes of nitrogen, such as ¹⁵N; isotopes of oxygen, such as ¹⁷O and ¹⁸O; isotopes of phosphorus, such as ³¹P and ³²P; isotopes of sulfur, such as .sup.³⁵S; isotopes of fluorine, such as ¹⁸F; and isotopes of chlorine, such as ³⁶Cl. Use of isotopic variations (e.g., deuterium, ²H) may afford certain therapeutic advantages resulting from greater metabolic stability, for example, increased in vivo half-life or reduced dosage requirements. Additionally, certain isotopic variations of the disclosed compounds may incorporate a radioactive isotope (e.g., tritium, ³H, or ¹⁴C), which may be useful in drug and/or substrate tissue distribution studies.

### EMBODIMENTS OF THE INVENTION

The following paragraphs present a number of embodiments of compounds of the invention. In each instance the embodiment includes both the recited compounds, as well as a single stereoisomer or mixture of stereoisomers thereof, as well as a pharmaceutically acceptable salt thereof.

### Embodiments (A1)

In one embodiment, the Compound of Formula I is that where R^{5a} is hydrogen or alkyl and R^{5c}, R^{5d}, R^{5e}, R^{5f}, and R^{5g} are hydrogen; and all other groups are independently as defined in the Summary of the Invention for a Compound of Formula I. In another embodiment, the Compound of Formula I is that where R^{5a} is alkyl and R^{5c}, R^{5d}, R^{5e}, R^{5f}, and R^{5g} are hydrogen; and all other groups are independently as defined in the Summary of the Invention for a Compound of Formula I.

### Embodiments (A2)

In another embodiment, the Compound of Formula I is that where R^{5b} is hydrogen, amino, or halo and R^{5a}, R^{5c}, R^{5d}, R^{5e}, R^{5f}, R^{5g}, and R^{5h} are hydrogen; and all other groups are independently as defined in the Summary of the Invention for a Compound of Formula I. In another embodiment, the Compound of Formula I is that where R^{5b} is halo and R^{5a}, R^{5c}, R^{5d}, R^{5e}, R^{5f}, R^{5g}, and R^{5h} are hydrogen; and all other groups are independently as defined in the Summary of the Invention for a Compound of Formula I. In another embodiment, the Compound of Formula I is that where R^{5b} is fluoro and R^{5a}, R^{5c}, R^{5d}, R^{5e}, R^{5f}, R^{5g}, and R^{5h} are hydrogen; and all other groups are independently as defined in the Summary of the Invention for a Compound of Formula I. In another embodiment, the Compound of Formula I is that where R^{5b} is amino; R^{5a}, R^{5c}, R^{5d}, R^{5e}, R^{5f}, R^{5g}, and R^{5h} are hydrogen; and all other groups are independently as defined in the Summary of the Invention for a Compound of Formula I.

### Embodiments (A3)

In another embodiment, the Compound of Formula I is that where R^{5c} is hydrogen or alkyl and R^{5a}, R^{5d}, R^{5e}, R^{5f}, and R^{5g} are hydrogen; and all other groups are independently as defined in the Summary of the Invention for a Compound of Formula I. In another embodiment, the Compound of Formula I is that where R^{5c} is alkyl and R^{5a}, R^{5d}, R^{5e}, R^{5f}, and R^{5g} are hydrogen; and all other groups are independently as defined in the Summary of the Invention for a Compound of Formula I.

### Embodiments (A4)

In another embodiment, the Compound of Formula I is that where R^{5h} is hydrogen or halo and R^{5a}, R^{5c}, R^{5d}, R^{5e}, R^{5f}, R^{5g}, and R^{5b} are hydrogen; and all other groups are independently as defined in the Summary of the Invention for a Compound of Formula I. In another embodiment, the Compound of Formula I is that where R^{5h} is halo and R^{5a}, R^{5c}, R^{5d}, R^{5e}, R^{5f}, R^{5g}, and R^{5b} are hydrogen; and all other groups are independently as defined in the Summary of the Invention for a Compound of Formula I. In another embodiment, the Compound of Formula I is that where R^{5h} is fluoro and R^{5a}, R^{5c}, R^{5d}, R^{5e}, R^{5f}, R^{5g}, and R^{5b} are hydrogen; and all other groups are independently as defined in the Summary of the Invention for a Compound of Formula I.

Another embodiment of the Invention is directed to a Compound of Formula I(a)

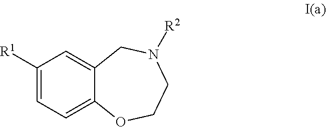

where R¹ and R² are independently as defined in the Summary of the Invention for a Compound of Formula I.

### Embodiment (1)

In another embodiment, the Compound of Formula I(a) is that where
R¹ is phenyl optionally substituted with one, two, or three R⁶ groups; or
R¹ is heteroaryl optionally substituted with one, two, or three R⁷;
R² is heteroaryl substituted with R³, R^{3a}, R^{3b}, R^{3c}, and R^{3d};
R³, R^{3a}, R^{3b}, R^{3c}, and R^{3d} are independently hydrogen; cyano; alkyl; alkenyl; halo; haloalkyl; hydroxyalkyl; alkoxyalkyl; cyanoalkyl; SR¹²; —S(O)₂R²⁰; carboxy; alkoxycarbonyl; halocarbonyl; —NR¹¹R^{11a}; —OR^{11a}; phenyl optionally substituted with one or two groups which are independently alkyl or halo; phenylalkyl optionally substituted with one or two R¹⁹; cycloalkyl; cycloalkylalkyl; heterocycloalkyl optionally substituted with one or two groups which are independently alkyl, alkoxycarbonyl, or benzyloxycarbonyl; heterocycloalkylalkyl optionally substituted with one or tow groups which are independently alkyl, alkoxycarbonyl, or benzyloxycarbonyl; heteroaryl; heteroarylalkyl; or alkyl substituted with one or two R¹⁶; or
two of R³, R^{3a}, R^{3b}, R^{3c}, and R^{3d}, when attached to the same carbon, form a cycloalkyl or a heterocycloalkyl; and the other of R³, R^{3a}, R^{3b}, R^{3c}, and R^{3d} are hydrogen;
each R⁶, when R⁶ is present, is independently nitro; cyano; halo; alkyl; halo; haloalkyl; —OR^{8a}; —NR⁸R^{8a}; —C(O)NR⁸R^{8a}; —S(O)₂R⁸; —NR⁸C(O)R⁹; —NR⁸S(O)₂R^{8a}; —NHC(O)NHR⁹; carboxy, —C(O)OR⁹; or heteroaryl optionally substituted with 1, 2, or 3 R¹⁴;
each R⁷, when R⁷ is present, is independently oxo; nitro; cyano; alkyl; alkenyl; halo; haloalkyl; hydroxyalkyl; alkoxyalkyl; —OR^{8a}; —SR¹³; —S(O)R¹³; —S(O)₂R^{13a}; —NR⁸R^{8a}; —C(O)NR⁸R^{8a}; —NR⁸C(O)OR⁹; —NR⁸C(O)R⁹; —NR⁸S(O)₂R^{8a}; —NR⁸C(O)NR^{8a}R⁹; —C(O)OR⁹; halocarbonyl; —S(O)₂NR⁸R⁹; alkylsulfonylalkyl; alkyl substituted with one or two —NR⁸R^{8a}; alkyl substituted with one or two —NR⁸C(O)R^{8a}; alkyl substituted with one or two —NR⁸C(O)OR⁹; alkyl substituted with one or two —S(O)₂R^{13a}; cycloalkyl; cycloalkylalkyl; heterocycloalkyl optionally substituted with one or two groups which are independently alkyl or amino; phenyl; phenylalkyl; heterocycloalkylalkyl; heteroaryl; or heteroarylalkyl;
R⁸, R¹¹, R¹⁵, R¹⁷, and R¹⁸ are independently hydrogen, alkyl, alkenyl, alkynyl, hydroxyalkyl, alkoxyalkyl, or haloalkyl;
R^{8a}; R^{11a}; and R^{15a} are independently hydrogen; alkyl; alkenyl; alkynyl; haloalkyl; hydroxyalkyl; cyanoalkyl; aminoalkyl; alkylaminoalkyl; dialkylaminoalkyl; alkoxyalkyl; carboxyalkyl; cycloalkyl; cycloalkylalkyl; heterocycloalkyl optionally substituted with one or two groups which are independently alkyl, alkoxycarbonyl, or benzyloxy; heterocycloalkylalkyl optionally substituted with one or two groups which are independently alkyl, alkoxycarbonyl, or benzyloxy; phenyl optionally substituted with one or two groups which are independently halo, alkyl, or alkoxy; phenylalkyl; heteroaryl; or heteroarylalkyl;
R⁹ is hydrogen; alkyl; alkenyl; alkynyl; hydroxyalkyl; alkoxyalkyl; aminoalkyl; alkylaminoalkyl; dialkylaminoalkyl; haloalkyl; hydroxyalkyl substituted with one, two, or three groups which are independently halo, amino, alkylamino, or dialkylamino; alkyl substituted with one or two aminocarbonyl; phenyl; phenylalkyl; cycloalkyl; cycloalkylalkyl optionally substituted with one or two groups which are independently amino or alkyl; heterocycloalkyl optionally substituted with one or two groups which are independently alkyl, alkoxycarbonyl, or benzyloxy; or heterocycloalkylalkyl optionally substituted with one or two groups which are independently alkyl, alkoxycarbonyl, or benzyloxy;
R¹² is alkyl or phenylalkyl;
R¹³ is alkyl, hydroxyalkyl, or haloalkyl; and
R^{13a} is hydroxy, alkyl, haloalkyl, hydroxyalkyl, or heterocycloalkyl optionally substituted with one or two groups which are independently halo, amino, alkylamino, dialkylamino, hydroxy, alkyl, or hydroxyalkyl;
each R¹⁴, when R¹⁴ is present, is independently amino, alkylamino, dialkylamino, acylamino, halo, hydroxy, alkyl, haloalkyl, hydroxyalkyl, aminoalkyl, alkylaminoalkyl, dialkylaminoalkyl, alkoxycarbonyl, aminocarbonyl, alkylaminocarbonyl, dialkylaminocarbonyl, or phenyl;
each R¹⁶ is independently —NR¹¹R^{11a}, —NR¹⁵S(O)R^{15a}, —OC(O)R¹⁷, or —OR¹⁸;
each R¹⁹ is independently halo, alkyl, haloalkyl, amino, alkylamino, dialkylamino, or alkoxy; and
R²⁰ is amino, alkylamino, dialkylamino, or heterocycloalkyl.

### Embodiment (B)

In another embodiment, the Compound of Formula I(a) is that where R¹ is heteroaryl optionally substituted with one, two, or three R⁷ groups; where each R⁷ independently of each other (when R⁷ is present) and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is 3,4-dihydro-2H-pyrido[3,2-b][1,4]oxazinyl, pyrido[2,3-b]pyrazinyl, imidazo[1,2-a]pyrimidinyl, imidazo[1,2-a]pyridinyl, triazolo[1,5-a]pyridinyl, indolyl, 2,3-dihydrobenzofuranyl, benzo[b]thienyl, quinolinyl, benzimidazolyl, indazolyl, 1H-pyrrolo[2,3-b]pyridinyl, pyridinyl, pyrimidinyl, pyridazinyl, thienyl, thiazolyl, benzothiazolyl, imidazopyridinyl, pyrazolopyridinyl, pyrrolopyridinyl, or thiazolopyridinyl, where R¹ is optionally substituted with one, two, or three R⁷; where each R⁷ independently of each other (when R⁷ is present) and all other groups are independently as defined in the Summary of the Invention for a Compound of Formula I or as defined in any of Embodiments (A1), (A2), (A3), (A4), and (1).

### Embodiments (H1)

In another embodiment, the Compound is according to Formula I(a) where R¹ is a 9-membered heteroaryl optionally substituted with one, two, or three R⁷; where each R⁷ independently of each other (when R⁷ is present) and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is benzimidazolyl, imidazo[4,5-b]pyridinyl, imidazo[4,5-c]pyridinyl, 3H-imidazo[4,5-c]pyridinyl, indazolyl, 1H-pyrazolo[3,4-b]pyridinyl, indolyl, 1H-pyrrolo[2,3-b]pyridinyl, 1H-pyrrolo[3,2-b]pyridinyl, benzo[d]thiazolyl, thiazolo[4,5-b]pyridinyl, thiazolo[4,5-c]pyridinyl, thiazolo[5,4-c]pyridinyl, or thiazolo[5,4-b]pyridinyl, and R¹ is optionally substituted with one, two, or three R⁷; where each R⁷ independently of each other (when R⁷ is present) and all other groups are independently as defined in the Summary of the Invention for a Compound of Formula I or as defined in any of Embodiments (A1), (A2), (A3), (A4), and (1).

### Embodiments (B1)

In another embodiment, the Compound is according to Formula I(a) where R¹ is 3H-imidazo[4,5-b]pyridinyl, 1H-imidazo[4,5-b]pyridinyl, 3H-imidazo[4,5-c]pyridinyl, or 1H-imidazo[4,5-c]pyridinyl, where R¹ is optionally substituted with one, two, or three R⁷ groups; where each R⁷ independently of each other (when R⁷ is present) and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is 3H-imidazo[4,5-b]pyridin-5-yl, 1H-imidazo[4,5-b]pyridin-5-yl, 3H-imidazo[4,5-b]pyridin-6-yl, 1H-imidazo[4,5-b]pyridin-6-yl, 3H-imidazo[4,5-c]pyridin-6-yl, 1H-imidazo[4,5-c]pyridin-6-yl, 3H-imidazo[4,5-c]pyridin-5-yl, or 1H-imidazo[4,5-c]pyridin-5-yl, where R¹ is optionally substituted with one, two, or three R⁷ groups; where each R⁷ independently of each other (when R⁷ is present) and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is 3H-imidazo[4,5-b]pyridin-5-yl, 1H-imidazo[4,5-b]pyridin-5-yl, 3H-imidazo[4,5-b]pyridin-6-yl, 1H-imidazo[4,5-b]pyridin-6-yl, 3H-imidazo[4,5-c]pyridin-6-yl, 1H-imidazo[4,5-c]pyridin-6-yl, 3H-imidazo[4,5-c]pyridin-5-yl, or 1H-imidazo[4,5-c]pyridin-5-yl, where R¹ is optionally substituted with one or two R⁷; each R⁷, when R⁷ is present, is independently halo, alkyl, cycloalkyl, haloalkyl, hydroxyalkyl, alkoxyalkyl, alkyl substituted with one or two —NR⁸R^{8a}, alkyl substituted with one or two —NR⁸C(O)OR⁹, —NR⁸R^{8a}, or —NR⁸C(O)OR⁹; and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is 3H-imidazo[4,5-b]pyridin-5-yl, 1H-imidazo[4,5-b]pyridin-5-yl, 3H-imidazo[4,5-b]pyridin-6-yl, 1H-imidazo[4,5-b]pyridin-6-yl, 3H-imidazo[4,5-c]pyridin-6-yl, 1H-imidazo[4,5-c]pyridin-6-yl, 3H-imidazo[4,5-c]pyridin-5-yl, or 1H-imidazo[4,5-c]pyridin-5-yl, where R¹ is optionally substituted with one or two R⁷; each R⁷, when R⁷ is present, is independently halo, alkyl, cycloalkyl, haloalkyl, hydroxyalkyl, alkoxyalkyl, alkyl substituted with one or two —NR⁸R^{8a}, alkyl substituted with one or two —NR⁸C(O)OR⁹, —NR⁸R^{8a}, or —NR⁸C(O)OR⁹; R⁸ and R^{8a} are independently hydrogen or alkyl; R⁹ is alkyl, benzyl, or haloalkyl; and all other groups are independently as defined in the Summary of the Invention for a Compound of Formula I or as defined in any of Embodiments (A1), (A2), (A3), (A4), and (1).

### Embodiments (B2)

In another embodiment, the Compound is according to Formula I(b1) or I(b2)

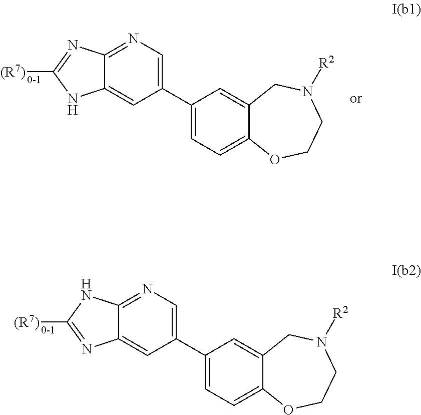

where R⁷, when R⁷ is present, is halo, alkyl, cycloalkyl, haloalkyl, hydroxyalkyl, alkoxyalkyl, alkyl substituted with one or two —NR⁸R^{8a}, alkyl substituted with one or two —NR⁸C(O)OR⁹, —NR⁸R^{8a}, or —NR⁸C(O)OR⁹; and R² and all other groups are independently as defined in the Summary of the Invention for a Compound of Formula I or as defined in any of Embodiments (A1), (A2), (A3), (A4), and (1). In another embodiment, the Compound is according to Formula I(b1) or I(b2), where R⁷, when R⁷ is present, is alkyl, cycloalkyl, haloalkyl, hydroxyalkyl, alkyl substituted with one or two —NR⁸C(O)OR⁹, —NR⁸R^{8a}, or —NR⁸C(O)OR⁹; R⁸ is hydrogen or alkyl; R^{8a} is hydrogen, alkyl, or haloalkyl; R⁹ is alkyl or benzyl; and R² and all other groups are independently as defined in the Summary of the Invention for a Compound of Formula I or as defined in any of Embodiments (A1), (A2), (A3), (A4), and (1). In another embodiment, the Compound is according to Formula I(b1) or I(b2), where R⁷, when R⁷ is present, is methyl, ethyl, n-propyl, isopropyl, cyclopropyl, cyclobutyl, monofluoromethyl, difluoromethyl, trifluoromethyl, 1-hydroxyethyl, 2-hydroxyethyl, amino, methylamino, ethylamino, methoxycarbonylamino, benzyloxycarbonylamino, aminomethyl, methylaminomethyl, or dimethylaminomethyl; and R² and all other groups are independently as defined in the Summary of the Invention for a Compound of Formula I or as defined in any of Embodiments (A1), (A2), (A3), (A4), and (1).

### Embodiments (B3)

In another embodiment, the Compound of Formula I is according to Formula I(a) where R¹ is benzo[d]thiazolyl, thiazolo[5,4-b]pyridinyl, thiazolo[5,4-c]pyridinyl, thiazolo[4,5-b]pyridinyl, or thiazolo[4,5-c]pyridinyl, where R¹ is optionally substituted with one, two, or three R⁷ groups; where all other groups and each R⁷, when R⁷ is present, are independently as defined in the Summary of the Invention for a Compound of Formula I or as defined in any of Embodiments (A1), (A2), (A3), (A4), and (1). In another embodiment, the Compound of Formula I is according to Formula I(a) where R¹ is benzo[d]thiazol-5-yl, benzo[d]thiazol-6-yl, thiazolo[5,4-b]pyridin-5-yl, thiazolo[5,4-b]pyridin-6-yl, thiazolo[5,4-c]pyridin-6-yl, thiazolo[4,5-b]pyridin-5-yl, thiazolo[4,5-b]pyridin-6-yl, or thiazolo[4,5-c]pyridin-6-yl, where R¹ is optionally substituted with one, two, or three R⁷ groups; where all other groups and each R⁷, when R⁷ is present, are independently as defined in the Summary of the Invention for a Compound of Formula I or as defined in any of Embodiments (A1), (A2), (A3), (A4), and (1). In another embodiment, the Compound of Formula I is according to Formula I(a) where R¹ is thiazolo[5,4-b]pyridin-6-yl or thiazolo[4,5-b]pyridin-6-yl optionally substituted with one R⁷ where R⁷ is alkyl, —NR⁸R^{8a}, or —NR⁸C(O)OR⁹; and other groups are independently as defined in the Summary of the Invention for a Compound of Formula I or as defined in any of Embodiments (A1), (A2), (A3), (A4), and (1). In another embodiment, the Compound of Formula I is according to Formula I(a) where R¹ is thiazolo[5,4-b]pyridin-6-yl or thiazolo[4,5-b]pyridin-6-yl optionally substituted with one R⁷ where R⁷ is —NR⁸R^{8a}; and other groups are independently as defined in the Summary of the Invention for a Compound of Formula I or as defined in any of Embodiments (A1), (A2), (A3), (A4), and (1). In another embodiment, the Compound of Formula I is according to Formula I(a) where R¹ is thiazolo[5,4-b]pyridin-6-yl or thiazolo[4,5-b]pyridin-6-yl optionally substituted with one R⁷ where R⁷ is alkyl, —NR⁸R^{8a}, or —NR⁸C(O)OR⁹; each R⁸, R^{8a}, and R⁹, independently of each other, are hydrogen or alkyl; and other groups are independently as defined in the Summary of the Invention for a Compound of Formula I or as defined in any of Embodiments (A1), (A2), (A3), (A4), and (1).

### Embodiments (B4)

In another embodiment, the Compound is according to Formula I(c1) or I(c2)

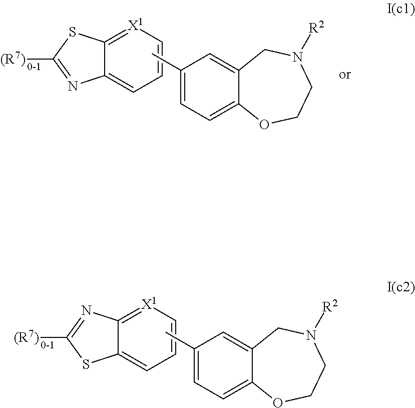

where X¹ is N or CH; R⁷ (when present), R², and all other groups are independently as defined in the Summary of the Invention for a Compound of Formula I or as defined in any of Embodiments (A1), (A2), (A3), (A4), and (1). In another embodiment, the Compound is according to Formula I(c1) or I(c2) where X¹ is N or CH; R⁷, when R⁷ is present, is alkyl, —NR⁸R^{8a}, or —NR⁸C(O)R⁹; and R² and all other groups are independently as defined in the Summary of the Invention for a Compound of Formula I or as defined in any of Embodiments (A1), (A2), (A3), (A4), and (1). In another embodiment, the Compound is according to Formula I(c1) or I(c2) where X¹ is N or CH; R⁷, when R⁷ is present, is alkyl, —NR⁸R^{8a}, or —NR⁸C(O)R⁹; each R⁸ and R^{8a} are independently hydrogen or alkyl and R⁹ is alkyl; and R² and all other groups are independently as defined in the Summary of the Invention for a Compound of Formula I or as defined in any of Embodiments (A1), (A2), (A3), (A4), and (1). In another embodiment, the Compound of Formula I is according to Formula I(c1) or I(c2) where X¹ is N or CH; R⁷, when R⁷ is present, is C₁₋₃-alkyl, amino, or C₁₋₃-alkylcarbonylamino; and R² and all other groups are independently as defined in the Summary of the Invention for a Compound of Formula I or as defined in any of Embodiments (A1), (A2), (A3), (A4), and (1). In another embodiment, the Compound is according to Formula I(c1) or I(c2) where X¹ is N or CH; R⁷, when R⁷ is present, is —NR⁸R^{8a} where R⁸ and R^{8a} are independently hydrogen or alkyl; and R² and all other groups are independently as defined in the Summary of the Invention for a Compound of Formula I or as defined in any of Embodiments (A1), (A2), (A3), (A4), and (1). In another embodiment, the Compound is according to Formula I(c1) or I(c2) where X¹ is N or CH; R⁷, when R⁷ is present, is —NR⁸R^{8a} where R⁸ and R^{8a} are independently hydrogen or C₁₋₃-alkyl; and R² and all other groups are independently as defined in the Summary of the Invention for a Compound of Formula I or as defined in any of Embodiments (A1), (A2), (A3), (A4), and (1).

### Embodiments (B4a)

In another embodiment, the Compound of Formula I is according to Formula I(c1) or I(c2) where X¹ is N; R⁷ (when present), R² and all other groups are independently as defined in the Summary of the Invention for a Compound of Formula I or as defined in any of Embodiments (A1), (A2), (A3), (A4), and (1). In another embodiment, the Compound of Formula I is according to Formula I(c) where X¹ is N; R⁷, when R⁷ is present, is alkyl, —NR⁸R^{8a}, or —NR⁸C(O)R⁹; and R² and all other groups are independently as defined in the Summary of the Invention for a Compound of Formula I or as defined in any of Embodiments (A1), (A2), (A3), (A4), and (1). In another embodiment, the Compound of Formula I is according to Formula I(c1) or I(c2) where X¹ is N; R⁷, when R⁷ is present, is alkyl, —NR⁸R^{8a}, or —NR⁸C(O)R⁹; each R⁸ and R^{8a} are independently hydrogen or alkyl and R⁹ is alkyl; and R² and all other groups are independently as defined in the Summary of the Invention for a Compound of Formula I or as defined in any of Embodiments (A1), (A2), (A3), (A4), and (1). In another embodiment, the Compound of Formula I is according to Formula I(c1) or I(c2) where X¹ is N; R⁷, when R⁷ is present, is C₁₋₃-alkyl, amino, or C₁₋₃-alkylcarbonylamino; and R² and all other groups are independently as defined in the Summary of the Invention for a Compound of Formula I or as defined in any of Embodiments (A1), (A2), (A3), (A4), and (1). In another embodiment, the Compound of Formula I is according to Formula I(c1) or I(c2) where X¹ is N; R⁷, when R⁷ is present, is —NR⁸R^{8a}; each R⁸ and R^{8a} are independently hydrogen or alkyl; and R² and all other groups are independently as defined in the Summary of the Invention for a Compound of Formula I or as defined in any of Embodiments (A1), (A2), (A3), (A4), and (1). In another embodiment, the Compound of Formula I is according to Formula I(c1) or I(c2) where X¹ is N; R⁷, when R⁷ is present, is —NR⁸R^{8a}; each R⁸ and R^{8a} are independently hydrogen or C₁₋₃-alkyl; and R² and all other groups are independently as defined in the Summary of the Invention for a Compound of Formula I or as defined in any of Embodiments (A1), (A2), (A3), (A4), and (1).

### Embodiments (B4b)

In another embodiment, the Compound of Formula I is according to Formula I(c1) or I(c2) where X¹ is C; R⁷ (when present), R², and all other groups are independently as defined in the Summary of the Invention for a Compound of Formula I or as defined in any of Embodiments (A1), (A2), (A3), (A4), and (1). In another embodiment, the Compound of Formula I is according to Formula I(c1) or I(c2) where X¹ is C; R⁷, when R⁷ is present, is alkyl, —NR⁸R^{8a}, or —NR⁸C(O)R⁹; and R² and all other groups are independently as defined in the Summary of the Invention for a Compound of Formula I or as defined in any of Embodiments (A1), (A2), (A3), (A4), and (1). In another embodiment, the Compound of Formula I is according to Formula I(c1) or I(c2) where X¹ is C; R⁷, when R⁷ is present, is alkyl, —NR⁸R^{8a}, or —NR⁸C(O)R⁹; each R⁸ and R^{8a} are independently hydrogen or alkyl and R⁹ is alkyl; and R² and all other groups are independently as defined in the Summary of the Invention for a Compound of Formula I or as defined in any of Embodiments (A1), (A2), (A3), (A4), and (1). In another embodiment, the Compound of Formula I is according to Formula I(c1) or I(c2) where X¹ is C; R⁷, when R⁷ is present, is C₁₋₃-alkyl, amino, or C₁₋₃-alkylcarbonylamino; and R² and all other groups are independently as defined in the Summary of the Invention for a Compound of Formula I or as defined in any of Embodiments (A1), (A2), (A3), (A4), and (1). In another embodiment, the Compound of Formula I is according to Formula I(c1) or I(c2) where X¹ is C; R⁷, when R⁷ is present, is —NR⁸R^{8a}; each R⁸ and R^{8a} are independently hydrogen or alkyl; and R² and all other groups are independently as defined in the Summary of the Invention for a Compound of Formula I or as defined in any of Embodiments (A1), (A2), (A3), (A4), and (1). In another embodiment, the Compound of Formula I is according to Formula I(c1) or I(c2) where X¹ is C; R⁷, when R⁷ is present, is —NR⁸R^{8a}; each R⁸ and R^{8a} are independently hydrogen or C₁₋₃-alkyl; and R² and all other groups are independently as defined in the Summary of the Invention for a Compound of Formula I or as defined in any of Embodiments (A1), (A2), (A3), (A4), and (1).

### Embodiments (B5)

In another embodiment, the Compound of Formula I is according to Formula I(a) where R¹ is benzimidazolyl optionally substituted with one, two, or three R⁷ groups; where all other groups and each R⁷ independently of each other (when R⁷ is present) are independently as defined in the Summary of the Invention for a Compound of Formula I or as defined in any of Embodiments (A1), (A2), (A3), (A4), and (1). In another embodiment, the Compound of Formula I is according to Formula I(a) where R¹ is benzimidazolyl optionally substituted with one or two R⁷ groups; and all other groups and each R⁷ (when R⁷ is present) are independently as defined in the Summary of the Invention for a Compound of Formula I or as defined in any of Embodiments (A1), (A2), (A3), (A4), and (1). In another embodiment, the Compound of Formula I is according to Formula I(a) where R¹ is benzimidazolyl optionally substituted with one R⁷; and all other groups are independently as defined in the Summary of the Invention for a Compound of Formula I or as defined in any of Embodiments (A1), (A2), (A3), (A4), and (1).

### Embodiments (B6)

In another embodiment, the Compound of Formula I is according to Formula I(d1) or I(d2)

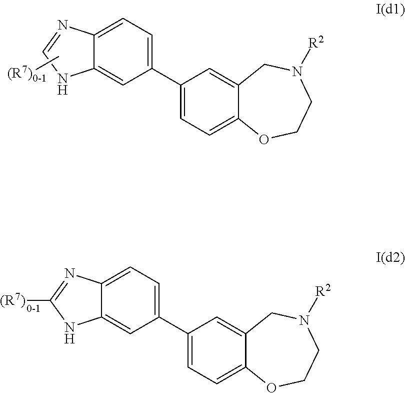

where R⁷, when R⁷ is present, is alkyl, haloalkyl, alkoxyalkyl, —SR¹³, —NR⁸R^{8a}, —NR⁸C(O)R⁹, —NR⁸C(O)OR⁹, —NR⁸C(O)NR^{8a}R⁹, cycloalkyl, heterocycloalkyl, or heteroaryl; and R² and all other groups are independently as defined in the Summary of the Invention for a Compound of Formula I or as defined in any of Embodiments (A1), (A2), (A3), (A4), and (1). In another embodiment, the Compound is according to Formula I(d1) or I(d2) where R⁷, when R⁷ is present, is alkyl, alkoxyalkyl, —SR¹³, —NR⁸R^{8a}, —NR⁸C(O)R⁹, —NR⁸C(O)OR⁹, cycloalkyl, heterocycloalkyl, or heteroaryl; R⁸ and R^{8a} are independently hydrogen or alkyl; R⁹ is alkyl, alkoxyalkyl, or optionally substituted heterocycloalkylalkyl; R¹³ is alkyl; and R² and all other groups are independently as defined in the Summary of the Invention for a Compound of Formula I or as defined in any of Embodiments (A1), (A2), (A3), (A4), and (1). In another embodiment, the Compound is according to Formula I(dl) or I(d2) where R⁷, when R⁷ is present, is alkyl, alkoxyalkyl, —SR¹³, —NR⁸R^{8a}, —NR⁸C(O)R⁹, —NR⁸C(O)OR⁹, cycloalkyl, heterocycloalkyl, or heteroaryl; R⁸ and R^{8a} are independently hydrogen or alkyl; R⁹ is alkyl; R¹³ is alkyl; and R² and all other groups are independently as defined in the Summary of the Invention for a Compound of Formula I or as defined in any of Embodiments (A1), (A2), (A3), (A4), and (1). In another embodiment, the Compound is according to Formula I(d1) or I(d2) where R⁷, when R⁷ is present, is C₁₋₃-alkyl, alkoxyalkyl, —SR¹³, —NR⁸R^{8a}, —NR⁸C(O)R⁹, —NR⁸C(O)OR⁹, cycloalkyl, heterocycloalkyl, or heteroaryl; R⁸ and R^{8a} are independently hydrogen or C₁₋₃-alkyl; R⁹ is C₁₋₃-alkyl; R¹³ is C₁₋₃-alkyl; and R² and all other groups are independently as defined in the Summary of the Invention for a Compound of Formula I or as defined in any of Embodiments (A1), (A2), (A3), (A4), and (1). In another embodiment, the Compound is according to Formula I(d1) or I(d2) where R⁷, when R⁷ is present, is methyl, ethyl, n-propyl, isopropyl, methoxymethyl, amino, methylamino, ethylamino, isopropylamino, dimethylamino, 3-piperidinylpropylcarbonylamino, methoxycarbonylamino, 2-(methoxy)-ethyloxycarbonylamino, cyclopropyl, cyclobutyl, cyclopentyl, cyclohexyl, azetidinyl, piperidinyl, or pyridinyl; and R² and all other groups are independently as defined in the Summary of the Invention for a Compound of Formula I or as defined in any of Embodiments (A1), (A2), (A3), (A4), and (1).

### Embodiment (B7)

In another embodiment, the Compound is according to Formula I(d1) or I(d2) where R⁷ is present and is alkyl; and R² and all other groups are independently as defined in the Summary of the Invention for a Compound of Formula I or as defined in any of Embodiments (A1), (A2), (A3), (A4), and (1). In another embodiment, the Compound is according to Formula I(d1) or I(d2) where R⁷ is present and is C₁₋₃-alkyl; and R² and all other groups are independently as defined in the Summary of the Invention for a Compound of Formula I or as defined in any of Embodiments (A1), (A2), (A3), (A4), and (1). In another embodiment, the Compound is according to Formula I(d1) or I(d2) where R⁷ is present and is —NR⁸R^{8a}; and all other groups are independently as defined in the Summary of the Invention for a Compound of Formula I or as defined in any of Embodiments (A1), (A2), (A3), (A4), and (1). In another embodiment, the Compound is according to Formula I(d1) or I(d2) where R⁷ is present and is —NR⁸R^{8a}; R⁸ and R^{8a} are independently hydrogen or alkyl; and all other groups are independently as defined in the Summary of the Invention for a Compound of Formula I or as defined in any of Embodiments (A1), (A2), (A3), (A4), and (1). In another embodiment, the Compound is according to Formula I(d1) or I(d2) where R⁷ is present and is —NR⁸R^{8a}; R⁸ and R^{8a} are independently hydrogen or C₁₋₃-alkyl; and all other groups are independently as defined in the Summary of the Invention for a Compound of Formula I or as defined in any of Embodiments (A 1), (A2), (A3), (A4), and (1). In another embodiment, the Compound is according to Formula I(dl) or I(d2) where R⁷ is present and is NR⁸C(O)OR⁹; and all other groups are independently as defined in the Summary of the Invention for a Compound of Formula I or as defined in any of Embodiments (A1), (A2), (A3), (A4), and (1). In another embodiment, the Compound is according to Formula I(d1) or I(d2) where R⁷ is present and is —NR⁸C(O)OR⁹; R⁸ and R⁹ are independently hydrogen or alkyl; and all other groups are independently as defined in the Summary of the Invention for a Compound of Formula I or as defined in any of Embodiments (A1), (A2), (A3), (A4), and (1). In another embodiment, the Compound is according to Formula I(d1) or I(d2) where R⁷ is present and is —NR⁸C(O)OR⁹; R⁸ and R⁹ are independently hydrogen or C₁₋₃-alkyl; and all other groups are independently as defined in the Summary of the Invention for a Compound of Formula I or as defined in any of Embodiments (A1), (A2), (A3), (A4), and (1). In another embodiment, the Compound is according to Formula I(d1) or I(d2) where R⁷ is present and is —SR¹³; and all other groups are independently as defined in the Summary of the Invention for a Compound of Formula I or as defined in any of Embodiments (A1), (A2), (A3), (A4), and (1).

In another embodiment, the Compound is according to Formula I(d1) or I(d2) where R⁷ is present and is haloalkyl; and all other groups are independently as defined in the Summary of the Invention for a Compound of Formula I or as defined in any of Embodiments (A 1), (A2), (A3), (A4), and (1). In another embodiment, the Compound is according to Formula I(d1) or I(d2) where R⁷ is present and is cycloalkyl; and all other groups are independently as defined in the Summary of the Invention for a Compound of Formula I or as defined in any of Embodiments (A1), (A2), (A3), (A4), and (1). In another embodiment, the Compound is according to Formula I(d1) or I(d2) where R⁷ is present and is cyclopropyl; and all other groups are independently as defined in the Summary of the Invention for a Compound of Formula I or as defined in any of Embodiments (A1), (A2), (A3), (A4), and (1).

### Embodiment (B8)

In another embodiment, the Compound is according to Formula I(f)

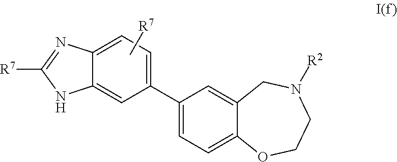

where the R⁷ at the 2-position is —NR⁸R^{8a} or —NR⁸C(O)OR⁹ and the other R⁷ is halo; and R² and all other groups are independently as defined in the Summary of the Invention for a Compound of Formula I or as defined in any of Embodiments (A1), (A2), (A3), (A4), and (1). In another embodiment, the Compound of Formula I is according to Formula I(f) where the R⁷ at the 2-position is —NR⁸R^{8a} or —NR⁸C(O)OR⁹ and the other R⁷ is halo; R⁸, R^{8a}, and R⁹ are independently hydrogen or alkyl; and R² and all other groups are independently as defined in the Summary of the Invention for a Compound of Formula I or as defined in any of Embodiments (A 1), (A2), (A3), (A4), and (1). In another embodiment, the Compound of Formula I is according to Formula I(f) where the R⁷ at the 2-position is —NR⁸R^{8a} or —NR⁸C(O)OR⁹ and the other R⁷ is halo; R⁸, R^{8a}, and R⁹ are independently hydrogen or C₁₋₃-alkyl; and R² and all other groups are independently as defined in the Summary of the Invention for a Compound of Formula I or as defined in any of Embodiments (A1), (A2), (A3), (A4), and (1). In another embodiment, the Compound is according to Formula I(f) where the R⁷ at the 2-position is methoxycarbonylamino or amino and the other the R⁷ is fluoro; and R² and all other groups are independently as defined in the Summary of the Invention for a Compound of Formula I or as defined in any of Embodiments (A1), (A2), (A3), (A4), and (1).

### Embodiment (B9)

In another embodiment, the Compound is according to Formula I(a) where R¹ is a 5-membered heteroaryl, where R¹ is optionally substituted with one or two R⁷; each R⁷ (when present), and all other groups are independently as defined in the Summary of the Invention for a Compound of Formula I or as defined in any of Embodiments (A1), (A2), (A3), (A4), and (1).

### Embodiments (B10)

In another embodiment, the Compound is according to Formula I(a) where R¹ is thiazol-2-yl, thiazol-4-yl, or thiazol-5-yl, where R¹ is optionally substituted with one or two R⁷; each R⁷ (when present), and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is thiazol-2-yl, thiazol-4-yl, or thiazol-5-yl, where R¹ is optionally substituted with one R⁷; R⁷, all other groups are independently as defined in the Summary of the Invention for a Compound of Formula I or as defined in any of Embodiments (A 1), (A2), (A3), (A4), and (1).

### Embodiments (B11)

In another embodiment, the Compound is according to Formula I(a) where R¹ is thiazol-2-yl, thiazol-4-yl, or thiazol-5-yl, where R¹ is optionally substituted with one or two R⁷; where each R⁷ (when present), where each R⁷ is independently alkyl, —NR⁸C(O)OR⁹, —C(O)NR⁸R^{8a}, or —NR⁸R^{8a}; each R⁸ and R^{8a} are independently hydrogen or alkyl and R⁹ is alkyl (in another embodiment each alkyl in R⁸, R^{8a}, and R⁹ are C₁₋₃-alkyl); and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is thiazol-2-yl, thiazol-4-yl, or thiazol-5-yl, where R¹ is optionally substituted with one or two R⁷; where each R⁷ (when present), where each R⁷ is independently alkyl, —NR⁸C(O)OR⁹, —C(O)NR⁸R^{8a}, or —NR⁸R^{8a}; each R⁸ and R^{8a} are independenly hydrogen or C₁₋₃-alkyl and R⁹ is C₁₋₃-alkyl; and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is thiazol-2-yl, thiazol-4-yl, or thiazol-5-yl, where R¹ is optionally substituted with one or two R⁷; each R⁷, when R⁷ is present, is independently methyl, or amino; and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is thiazol-2-yl, thiazol-4-yl, or thiazol-5-yl, where R¹ is substituted with two R⁷; where one R⁷, is alkyl and the other R⁷—NR⁸R^{8a}; and all other groups are independently as defined in the Summary of the Invention for a Compound of Formula I or as defined in any of Embodiments (A1), (A2), (A3), (A4), and (1).

### Embodiments (B12)

In another embodiment, the Compound is according to Formula I(a) where R¹ is thien-2-yl, thien-3-yl, thien-4-yl, or thien-5-yl, where R¹ is optionally substituted with one or two R⁷ groups; where each R⁷ (when present), and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is thien-2-yl, thien-3-yl, thien-4-yl, or thien-5-yl; and all other groups are independently as defined in the Summary of the Invention for a Compound of Formula I or as defined in any of Embodiments (A1), (A2), (A3), (A4), and (1).

### Embodiments (B13)

In another embodiment, the Compound is according to Formula I(a) where R¹ is pyrazol-1-yl, pyrazol-3-yl, pyrazol-4-yl, or pyrazol-5-yl, where R¹ is optionally substituted with one or two R⁷ groups; where each R⁷ (when present), and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is pyrazol-1-yl, pyrazol-3-yl, pyrazol-4-yl, or pyrazol-5-yl; and all other groups are independently as defined in the Summary of the Invention for a Compound of Formula I or as defined in any of Embodiments (A1), (A2), (A3), (A4), and (1).

### Embodiment (B14)

In another embodiment, the Compound is according to Formula I(a) where R¹ is a 6-membered heteroaryl, where R¹ is optionally substituted with one or two R⁷ groups; where each R⁷ (when present), and all other groups are independently as defined in the Summary of the Invention for a Compound of Formula I or as defined in any of Embodiments (A1), (A2), (A3), (A4), and (1).

### Embodiments (B15)

In another embodiment, the Compound is according to Formula I(a) where R¹ is pyrimidin-2-yl, pyrimidin-4-yl, pyrimidin-5-yl, pyrimidin-6-yl, where R¹ is optionally substituted with one or two R⁷ groups; where each R⁷ (when present), and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is pyrimidin-2-yl, pyrimidin-4-yl, pyrimidin-5-yl, pyrimidin-6-yl, where R¹ is optionally substituted with one R⁷ where R⁷ is —NR⁸R^{8a}; R⁸ and R^{8a} are independently hydrogen or alkyl; and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is pyrimidin-2-yl, pyrimidin-4-yl, pyrimidin-5-yl, pyrimidin-6-yl, where R¹ is optionally substituted with one R⁷ where R⁷ is —NR⁸R^{8a}; R⁸ and R^{8a} are independently hydrogen or C₁₋₃alkyl; and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is R¹ is 2-amino-pyrimidin-5-yl; and all other groups are independently as defined in the Summary of the Invention for a Compound of Formula I or as defined in any of Embodiments (A1), (A2), (A3), (A4), and (1).

### Embodiments (B16)

In another embodiment, the Compound is according to Formula I(a) where R¹ is pyridin-2-yl, pyridin-3-yl, pyridin-4-yl, pyridin-5-yl, or pyridin-6-yl, where R¹ is optionally substituted with one or two R⁷ groups; where each R⁷ (when present), and all other groups are independently as defined in the Summary of the Invention for a Compound of Formula I or as defined in any of Embodiments (A1), (A2), (A3), (A4), and (1). In another embodiment, the Compound is according to Formula I(a) where R′ is pyridinyl where R′ is optionally substituted with one or two R⁷ where each R⁷ is independently halo, cyano, alkylsulfonylalkyl, —OR^{8a}, —C(O)NR⁸R^{8a}, S(O)₂OH, —S(O)R¹³, S(O)₂R^{13a}, —S(O)₂NR⁸R⁹, —NR⁸R^{8a}, —NR⁸C(O)OR⁹, —NR⁸C(O)R⁹, —NR⁸S(O)₂R^{8a}, or heterocycloalkyl optionally substituted with one amino; and all other groups are independently as defined in the Summary of the Invention for a Compound of Formula I or as defined in any of Embodiments (A1), (A2), (A3), (A4), and (1).

### Embodiments (B16a)

In another embodiment, the Compound is according to Formula I(a) where R¹ is pyridinyl where R¹ is optionally substituted with one or two R⁷ where each R⁷ is independently halo, cyano, alkylsulfonylalkyl, —C(O)NR⁸R^{8a}, S(O)₂OH, —S(O)R¹³, —S(O)₂R^{13a}, —S(O)₂NR⁸R⁹, —NR⁸R^{8a}, —NR⁸C(O)OR⁹, —NR⁸C(O)R⁹, —NR⁸S(O)₂R^{8a}, heterocycloalkyl optionally substituted with one amino; where
each R⁸ is independently hydrogen, haloalkyl, or alkyl;
each R^{8a} is independently hydrogen, alkyl, benzyl, or phenyl which phenyl is optionally substituted with one or two groups which are independently halo or alkyl;
each R⁹ is independently hydrogen; alkyl; hydroxyalkyl; alkoxyalkyl; aminoalkyl; alkylaminoalkyl; dialkylaminoalkyl; haloalkyl; hydroxyalkyl substituted with one, two, or three halo, heterocycloalkylalkyl optionally substituted with one alkyl; heterocycloalkyl optionally substituted with one alkyl; cycloalkylalkyl optionally substituted with one amino; cycloalkyl;
R¹³ is alkyl or hydroxyalkyl;
R^{13a} is alkyl; hydroxyalkyl; heterocycloalkyl optionally substituted with one or two groups which are independently halo, amino, alkylamino, dialkylamino, hydroxy, alkyl, or hydroxyalkyl;
   and all other groups are independently as defined in the Summary of the Invention for a Compound of Formula I or as defined in any of Embodiments (A1), (A2), (A3), (A4), and (1).

### Embodiments (B16b)

In another embodiment, the Compound of Formula I is according to Formula I(e)

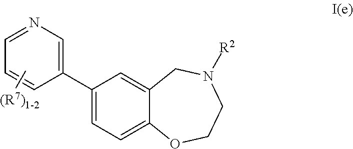

where each R⁷ and R² are independently as defined in the Summary of the Invention for a Compound of Formula I or as defined in any of Embodiments (A1), (A2), (A3), (A4), and (1). In another embodiment, the Compound of Formula I is according to Formula I(e) where each R⁷ is independently as defined in embodiment B16a and R² is as defined in the Summary of the Invention for a Compound of Formula I or as defined in any of Embodiments (A1), (A2), (A3), (A4), and (1).

### Embodiments (B16c)

In another embodiment, the Compound of Formula I is according to Formula I(e1)

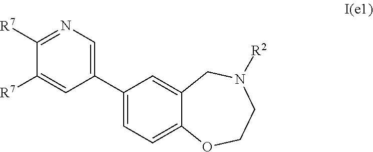

where each R⁷ and R² are independently as defined in the Summary of the Invention for a Compound of Formula I or as defined in any of Embodiments (A1), (A2), (A3), (A4), and (1). In another embodiment, the Compound of Formula I is according to Formula I(e) where each R⁷ is independently as defined in embodiment B16a and R² is as defined in the Summary of the Invention for a Compound of Formula I or as defined in any of Embodiments (A1), (A2), (A3), (A4), and (1). In another embodiment, the Compound of Formula I is according to Formula I(e1) where the R⁷ in the 2-position is hydrogen, halo, cyano, alkoxy, alkyl, or —NR⁸R^{8a} and the R⁷ in the 3-position is —NR⁸S(O)₂R^{8a}; and R² and all other groups are as defined in the Summary of the Invention for a Compound of Formula I or as defined in any of Embodiments (A1), (A2), (A3), (A4), and (1). In another embodiment, the Compound of Formula I is according to Formula I(e1) where the R⁷ in the 2-position is hydroxy or —NR⁸R^{8a} and the R⁷ in the 3-position is —S(O)R¹³, —S(O)₂R^{13a}, —S(O)₂NR⁸R⁹; and R² and all other groups are as defined in the Summary of the Invention for a Compound of Formula I or as defined in any of Embodiments (A1), (A2), (A3), (A4), and (1). In another embodiment, the Compound of Formula I is according to Formula I(e1) where the R⁷ in the 2-position is hydroxy or —NR⁸R^{8a} and the R⁷ in the 3-position is —S(O)R¹³, —S(O)₂R^{13a}, —S(O)₂NR⁸R⁹; R¹³ is hydroxyalkyl; R^{13a} is alkyl or heterocycloalkyl optionally substituted with one group which is amino, alkyl, hydroxyalkyl, or hydroxy; each R⁸ and R^{8a} are independently hydrogen or alkyl; R⁹ is hydrogen, haloalkyl, alkoxyalkyl, hydroxyalkyl, aminoalkyl, alkylaminoalkyl, dialkylaminoalkyl, cycloalkyl, heterocycloalkyl, heterocycloalkylalkyl, alkyl substituted with one aminocarbonyl, or hydroxyalkyl which is substituted with one amino or 3 halo; and R² and all other groups are as defined in the Summary of the Invention for a Compound of Formula I or as defined in any of Embodiments (A1), (A2), (A3), (A4), and (1).

### Embodiments (B17)

In another embodiment, the Compound of Formula I is according to Formula I(a) where R¹ is pyridazin-3-yl, pyridazin-4-yl, pyridazin-5-yl, or pyridazin-6-yl, where R¹ is optionally substituted with one or two R⁷ groups; where each R⁷ (when present), and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is pyridazin-3-yl, pyridazin-4-yl, pyridazin-5-yl, or pyridazin-6-yl, where R¹ is optionally substituted with one or two R⁷ groups where each R⁷ is independently —NR⁸R^{8a}; R⁸ and R^{8a} are independently hydrogen or alkyl; and R² and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is 3-amino-pyridazin-6-yl; and all other groups are independently as defined in the Summary of the Invention for a Compound of Formula I or as defined in any of Embodiments (A1), (A2), (A3), (A4), and (1).

### Embodiments (B18)

In another embodiment, the Compound is according to Formula I(a) where R¹ is pyrazin-2-yl, pyrazin-3-yl, pyrazin-5-yl, or pyrazin-6-yl, where R¹ is optionally substituted with one or two R⁷ groups; where each R⁷ (when present), and all other groups are independently as defined in the Summary of the Invention for a Compound of Formula I or as defined in any of Embodiments (A 1), (A2), (A3), (A4), and (1). In another embodiment, the Compound is according to Formula I(a) where R¹ is pyrazin-2-yl, pyrazin-3-yl, pyrazin-5-yl, or pyrazin-6-yl, where R¹ is optionally substituted with one R⁷ where R⁷ is —NR⁸R^{8a}; R⁸ and R^{8a} are independently hydrogen or alkyl; and R² and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is 5-amino-pyrazin-2-yl; and R² and all other groups are independently as defined in the Summary of the Invention for a Compound of Formula I or as defined in any of Embodiments (A1), (A2), (A3), (A4), and (1).

### Embodiments (B19)

In another embodiment, the Compound is according to Formula I(a) where R¹ is 1H-pyrrolo[2,3-b]pyridinyl, optionally substituted with one or two R⁷ groups; where each R⁷, when R⁷ is present, and all other groups are independently as defined in the Summary of the Invention for a Compound of Formula I or as defined in any of Embodiments (A 1), (A2), (A3), (A4), and (1). In another embodiment, the Compound is according to Formula I(a) where R¹ is 1H-pyrrolo[2,3-b]pyridin-5-yl, optionally substituted with one or two R⁷ groups; where each R⁷, when R⁷ is present, and all other groups are independently as defined in the Summary of the Invention for a Compound of Formula I or as defined in any of Embodiments (A 1), (A2), (A3), (A4), and (1). In another embodiment, the Compound is according to Formula I(a) where R¹ is 1H-pyrrolo[2,3-b]pyridin-5-yl, optionally substituted with one R⁷; where the R⁷, when R⁷ is present, and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is 1H-pyrrolo[2,3-b]pyridin-5-yl, optionally substituted with one R⁷; R⁷, when R⁷ is present, is methyl or ethyl; and all other groups are independently as defined in the Summary of the Invention for a Compound of Formula I or as defined in any of Embodiments (A1), (A2), (A3), (A4), and (1).

### Embodiments (B20)

In another embodiment, the Compound is according to Formula I(a) where R¹ is indazolyl, optionally substituted with one or two R⁷ groups; where R⁷, when R⁷ is present, and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is indazol-5-yl or indazol-6-yl, where R¹ is optionally substituted with one or two R⁷ groups; where R⁷, when R⁷ is present, and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is indazol-5-yl or indazol-6-yl, where R¹ is optionally substituted with one R⁷; R⁷, when present, is alkyl or amino; and R² and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is indazol-5-yl, indazol-6-yl, or N-methyl-indazol-5-yl; and all other groups are independently as defined in the Summary of the Invention for a Compound of Formula I or as defined in any of Embodiments (A1), (A2), (A3), (A4), and (1).

### Embodiment (B21)

In another embodiment, the Compound is according to Formula I(a) where R¹ is benzimidazolyl substituted with two R⁷ groups where each R⁷ is alkyl; and R² and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is benzimidazolyl substituted with two R⁷ groups where each R⁷ is C₁₋₃-alkyl; and R² and all other groups are independently as defined in the Summary of the Invention for a Compound of Formula I or as defined in any of Embodiments (A1), (A2), (A3), (A4), and (1).

### Embodiments (B22)

In another embodiment, the Compound is according to Formula I(a) where R¹ is quinolin-2-yl, quinolin-3-yl, quinolin-4-yl, quinolin-5-yl, quinolin-6-yl, quinolin-7-yl, quinolin-8-yl, isoquinolin-1-yl, isoquinolin-3-yl, isoquinolin-4-yl, isoquinolin-5-yl, isoquinolin-6-yl, isoquinolin-7-yl, isoquinolin-8-yl, quinazolin-2-yl, quinazolin-3-yl, quinazolin-5-yl, quinazolin-6-yl, quinazolin-7-yl, or quinazolin-8-yl, where R¹ is optionally substituted with one or two R⁷ groups; where each R⁷, when R⁷ is present, and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is quinolin-2-yl, quinolin-3-yl, quinolin-4-yl, quinolin-5-yl, quinolin-6-yl, quinolin-7-yl, quinolin-8-yl, quinazolin-2-yl, quinazolin-3-yl, quinazolin-5-yl, quinazolin-6-yl, quinazolin-7-yl, or quinazolin-8-yl; and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is quinolin-3-yl or quinazolin-6-yl; and R² and all other groups are independently as defined in the Summary of the Invention for a Compound of Formula I or as defined in any of Embodiments (A1), (A2), (A3), (A4), and (1).

### Embodiments (B24)

In another embodiment, the Compound is according to Formula I(a) where R¹ is 2,3-dihydrobenzofuran-4-yl, 2,3-dihydrobenzofuran-5-yl, 2,3-dihydrobenzofuran-6-yl, or 2,3-dihydrobenzofuran-7-yl, where R¹ is optionally substituted with one or two R⁷ groups; where each R⁷, when R⁷ is present, and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is 2,3-dihydrobenzofuran-4-yl, 2,3-dihydrobenzofuran-5-yl, 2,3-dihydrobenzofuran-6-yl, or 2,3-dihydrobenzofuran-7-yl; and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is 2,3-dihydrobenzofuran-5-yl; and R² and all other groups are independently as defined in the Summary of the Invention for a Compound of Formula I or as defined in any of Embodiments (A1), (A2), (A3), (A4), and (1).

### Embodiments (B25)

In another embodiment, the Compound is according to Formula I(a) where R¹ is indol-1-yl, indol-2-yl, indol-3-yl, indol-4-yl, indol-5-yl, indol-6-yl, or indol-7-yl, where R¹ is optionally substituted with one or two R⁷ groups; where each R⁷, when R⁷ is present, and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is indol-1-yl, indol-2-yl, indol-3-yl, indol-4-yl, indol-5-yl, indol-6-yl, or indol-7-yl where R¹ is optionally substituted with one R⁷ where R⁷ is alkyl; and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is indol-5-yl optionally substituted with one R⁷ where R⁷ is alkyl; and all other groups are independently as defined in the Summary of the Invention for a Compound of Formula I or as defined in any of Embodiments (A1), (A2), (A3), (A4), and (1).

### Embodiments (B26)

In another embodiment, the Compound is according to Formula I(a) where R¹ is [1,2,4]triazolo[1,5-a]pyridin-2-yl, [1,2,4]triazolo[1,5-a]pyridin-5-yl, [1,2,4]triazolo[1,5-a]pyridin-6-yl, [1,2,4]triazolo[1,5-a]pyridin-7-yl, or [1,2,4]triazolo[1,5-a]pyridin-8-yl, where R¹ is optionally substituted with one or two R⁷ groups; where each R⁷, when R⁷ is present, and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is [1,2,4]triazolo[1,5-a]pyridin-2-yl, [1,2,4]triazolo[1,5-a]pyridin-5-yl, [1,2,4]triazolo[1,5-a]pyridin-6-yl, [1,2,4]triazolo[1,5-a]pyridin-7-yl, or [1,2,4]triazolo[1,5-a]pyridin-8-yl, where R¹ is optionally substituted with one R⁷ where R⁷ is —NR⁸R^{8a}; R⁸ and R⁸ are independently hydrogen or alkyl; and R² and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is [1,2,4]triazolo[1,5-a]pyridin-6-yl, or [1,2,4]triazolo[1,5-a]pyridin-7-yl, optionally substituted with one R⁷ where R⁷ is amino; and all other groups are independently as defined in the Summary of the Invention for a Compound of Formula I or as defined in any of Embodiments (A1), (A2), (A3), (A4), and (1).

### Embodiments (B27)

In another embodiment, the Compound is according to Formula I(g)

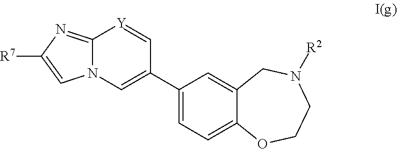

where Y is N or CH; and R² and R⁷ are independently as defined in the Summary of the Invention for a Compound of Formula I or as defined in any of Embodiments (A1), (A2), (A3), (A4), and (1). In another embodiment the Compound of Formula I(g) is that where R⁷, when present, is —NR⁸R^{8a} or —NR⁸C(O)R⁹; and R² and all other groups are independently as defined in the Summary of the Invention for a Compound of Formula I or as defined in any of Embodiments (A1), (A2), (A3), (A4), and (1). In another embodiment the Compound of Formula I(g) is that where R⁷, when present, is —NR⁸R^{8a} or —NR⁸C(O)R⁹; R⁸ and R^{8a} are independently hydrogen or alkyl; R⁹ is alkyl or haloalkyl; and R² and all other groups are independently as defined in the Summary of the Invention for a Compound of Formula I or as defined in any of Embodiments (A1), (A2), (A3), (A4), and (1). In another embodiment the Compound of Formula I(g) is that where R⁷, when present, is —NR⁸R^{8a} or —NR⁸C(O)R⁹; R⁸ and R^{8a} are independently hydrogen or C₁₋₃-alkyl; R⁹ is C₁₋₃-alkyl or halo-C₁₋₃-alkyl; and R² and all other groups are independently as defined in the Summary of the Invention for a Compound of Formula I or as defined in any of Embodiments (A1), (A2), (A3), (A4), and (1). In another embodiment the Compound of Formula I(g) is that where R⁷, when present, is amino or trifluoromethylcarbonylamino; and R² and all other groups are independently as defined in the Summary of the Invention for a Compound of Formula I or as defined in any of Embodiments (A1), (A2), (A3), (A4), and (1).

### Embodiments (B28)

In another embodiment, the Compound of Formula I is according to Formula I(a) where R¹ is pyrido[2,3-b]pyrazinyl optionally substituted with one or two R⁷ groups; where R⁷ and all other groups are independently as defined in the Summary of the Invention for a Compound of Formula I or as defined in any of Embodiments (A1), (A2), (A3), (A4), and (1). In another embodiment, the Compound of Formula I is according to Formula I(a) where R¹ is unsubstituted pyrido[2,3-b]pyrazinyl where all other groups are independently as defined in the Summary of the Invention for a Compound of Formula I or as defined in any of Embodiments (A1), (A2), (A3), (A4), and (1).

### Embodiments (B29)

In another embodiment, the Compound of Formula I is according to Formula I(a) where R¹ is 3,4-dihydro-2H-pyrido[3,2-b][1,4]oxazinyl optionally substituted with one or two R⁷ groups; where R⁷ and all other groups are independently as defined in the Summary of the Invention for a Compound of Formula I or as defined in any of Embodiments (A1), (A2), (A3), (A4), and (1). In another embodiment, the Compound of Formula I is according to Formula I(a) where R¹ is unsubstituted 3,4-dihydro-2H-pyrido[3,2-b][1,4]oxazinyl where all other groups are independently as defined in the Summary of the Invention for a Compound of Formula I or as defined in any of Embodiments (A1), (A2), (A3), (A4), and (1).

### Embodiments (C)

In another embodiment, the Compound of Formula I is according to Formula I(a) where R¹ is phenyl optionally substituted with one, two, or three R⁶ groups; where each R⁶, when R⁶ is present, and all other groups are independently as defined in the Summary of the Invention for a Compound of Formula I or as defined in any of Embodiments (A1), (A2), (A3), (A4), and (1). In another embodiment, the Compound of Formula I is according to Formula I(a) where R¹ is phenyl optionally substituted with one or two R⁶ groups; where each R⁶, when R⁶ is present, and all other groups are independently as defined in the Summary of the Invention for a Compound of Formula I or as defined in any of Embodiments (A1), (A2), (A3), (A4), and (1).

### Embodiments (C1)

In another embodiment, the Compound of Formula I is according to Formula I(a) where R¹ is phenyl optionally substituted with one, two, or three R⁶ groups; where each R⁶ is independently nitro, halo, alkoxy, —OR^{8a}, —S(O)₂R⁸, —NR⁸R^{8a}, —NR⁸S(O)₂R^{8a}, —NR⁸C(O)R⁹, —C(O)NR⁸R^{8a}, —NR⁸C(O)NR^{8a}R⁹, carboxy, alkoxycarbonyl, or heteroaryl optionally substituted with one or two R¹⁴; and all other groups are independently as defined in the Summary of the Invention for a Compound of Formula I or as defined in any of Embodiments (A1), (A2), (A3), (A4), and (1). In another embodiment, the Compound of Formula I is according to Formula I(a) where R¹ is phenyl optionally substituted with one, two, or three R⁶ groups; where each R⁶ is independently —S(O)₂R⁸, —C(O)NR⁸R^{8a} or heteroaryl optionally substituted with one or two R¹⁴; and all other groups are independently as defined in the Summary of the Invention for a Compound of Formula I or as defined in any of Embodiments (A1), (A2), (A3), (A4), and (1).

### Embodiment (C2)

In another embodiment, the Compound is according to Formula I(a) where R¹ is phenyl optionally substituted with one, two, or three R⁶ groups; where each R⁶ is independently nitro, halo, alkoxy, —OR^{8a}, —S(O)₂R⁸, —NR⁸R^{8a}, —NR⁸S(O)₂R^{8a}, —NR⁸C(O)R⁹, —C(O)NR⁸R^{8a}, —NR⁸C(O)NR^{8a}R⁹, carboxy, alkoxycarbonyl, or heteroaryl optionally substituted with one or two R¹⁴; each R⁸ is independently hydrogen or alkyl; each R^{8a} is independently hydrogen, alkyl, haloalkyl, optionally substituted cycloalkyl, or optionally substituted heterocycloalkyl; R⁹ is alkyl; R¹⁴, when present, is hydroxyalkyl; and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is phenyl optionally substituted with one, two, or three R⁶ groups; where each R⁶ is independently nitro, halo, alkoxy, —OR^{8a}, —S(O)₂R⁸, —NR⁸R^{8a}, —NR⁸S(O)₂R^{8a}, —NR⁸C(O)R⁹, —C(O)NR⁸R^{8a}, —NR⁸C(O)NR^{8a}R⁹, K carboxy, alkoxycarbonyl, or heteroaryl optionally substituted with one or two R¹⁴; each R⁸ is independently hydrogen or C₁₋₃-alkyl; each R^{8a} is independently hydrogen, alkyl, haloalkyl, optionally substituted cycloalkyl, or optionally substituted heterocycloalkyl; R⁹ is C₁₋₃-alkyl; R¹⁴, when present, is hydroxyalkyl; and all other groups are independently as defined in the Summary of the Invention for a Compound of Formula I or as defined in any of Embodiments (A1), (A2), (A3), (A4), and (1).

### Embodiment (C3)

In another embodiment, the Compound is according to Formula I(a) where R¹ is phenyl optionally substituted with one or two R⁶ groups where each R⁶ is independently nitro, chloro, methoxy, methylsulfonyl, amino, methylaminocarbonylamino, methylamino, carboxy, methylcarbonylamino, aminocarbonyl, methylaminocarbonyl, ethylaminocarbonyl, n-propylaminocarbonyl, isopropylaminocarbonyl, 2-monofluoroethylaminocarbonyl, 2,2-difluoroethylaminocarbonyl, 2,2,2-trifluoroethylaminocarbonyl, 1,1,1-trifluoroprop-2-ylaminocarbonyl, cyclopropylaminocarbonyl, pyrrolidinylaminocarbonyl, methoxycarbonyl, imidazolyl, imidazolyl substituted with hydroxymethyl, or pyrazolyl; and R² and all other groups are independently as defined in the Summary of the Invention for a Compound of Formula I or as defined in any of Embodiments (A1), (A2), (A3), (A4), and (1).

In a Compound as described by any one of Formula I, I(a), I(b1), I(b2), I(c1), I(c2), I(d1), I(d2), I(e1), I(e2), I(f), and I(g), or by any of the above embodiments (1), (A1), (A2), (A3), (A4), (B), (H1), (H2), (B1), (B2), (B3), (B4), (B4a), (B4b), (B5), (B6), (B8), (B9), (B10), (B11), (B12), (B13), (B14), (B15), (B16), (B16a), (B16b), (B16c), (B17), (B18), (B19), (B20), (B21), (B22), (B23), (B24), (B25), (B26), (B27), (C), (C1), (C2), and (C3), R² can be described according to any of the following embodiments.

### Embodiments (D)

In another embodiment, R² is a 6-membered heteroaryl substituted with R³, R^{3a}, R^{3b}, and R^{3c}; R³, R^{3a}, R^{3b}, and R^{3c} and all other groups are independently as defined in the Summary of the Invention for a Compound of Formula I or as defined in embodiment (1).

### Embodiments (D1)

In another embodiment, R² is pyrimidinyl substituted with R³, R^{3a}, and R^{3b}; where R³, R^{3a}, R^{3b}, and all other groups are independently as defined in the Summary of the Invention for a Compound of Formula I or as defined in embodiment (1).

### Embodiments (D2)

In another embodiment, R² is according to Formula (a)

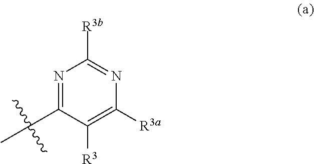

where R³, R^{3a}, and R^{3b} are independently hydrogen; alkyl; halo; hydroxyalkyl; cyanoalkyl; —NR¹¹R^{11a}; —S(O)₂R²⁰; optionally substituted cycloalkylalkyl; optionally substituted heterocycloalkyl; optionally substituted phenylalkyl; alkyl substituted with one or two R¹⁶; or —OR^{11a}; and all other groups are independently as defined in the Summary of the Invention for a Compound of Formula I or as defined in embodiment (1). In another embodiment, R² is according to Formula (a) where R³, R^{3a}, and R^{3b} are independently hydrogen; alkyl; halo; hydroxyalkyl; cyanoalkyl; —NR¹¹R^{11a}; —S(O)₂R²⁰; cycloalkylalkyl; heterocycloalkyl optionally substituted with one or two alkyl; phenylalkyl optionally substituted with one or two R¹⁹; alkyl substituted with one or two R¹⁶; or —OR^{11a}; and all other groups are independently as defined in the Summary of the Invention for a Compound of Formula I or as defined in embodiment (1). In another embodiment, R² is according to Formula (a) where R³, R^{3a}, and R^{3b} are independently hydrogen; alkyl; halo; hydroxyalkyl; cyanoalkyl; —NR¹¹R^{11a}; —S(O)₂R²⁰; cycloalkylalkyl; heterocycloalkyl optionally substituted with one or two alkyl; phenylalkyl optionally substituted with one or two R¹⁹; alkyl substituted with one or two R¹⁶; or —OR^{11a}; each R¹⁹ is independently halo, alkyl, haloalkyl, alkoxy, amino, alkylamino, or dialkylamino; each R¹⁶ is independently —NR¹¹R^{11a} or —OC(O)R¹⁷; R¹⁷ is alkyl; each R^{11a} is independently hydrogen, alkyl (in another embodiment each alkyl is C₁₋₃-alkyl), or cycloalkyl; each R^{11a} is independently hydrogen; alkyl (in another embodiment each alkyl is C₁₋₃-alkyl); aminoalkyl; alkylaminoalkyl; dialkylaminoalkyl; phenyl; phenyl substituted with one alkoxy; phenylalkyl; heterocycloalkyl; heterocycloalkyl substituted with one or two alkyl; heterocycloalkylalkyl; heterocycloalkylalkyl substituted with one or two alkyl; R²⁰ is amino, alkylamino, dialkylamino, or heterocycloalkyl; and all other groups are independently as defined in the Summary of the Invention for a Compound of Formula I or as defined in embodiment (1). In another embodiment, R² is according to Formula (a) where R³, R^{3a}, and R^{3b} are independently hydrogen; alkyl (in another embodiment alkyl is C₁₋₃-alkyl); phenylalkyl optionally substituted with one or two groups which are independently halo, haloalkyl, alkoxy, amino, alkylamino, or dialkylamino; —NR¹¹R^{11a}; heterocycloalkyl; cycloalkylalkyl; alkyl substituted with one or two R¹⁶; or hydroxyalkyl; where each R¹¹ is independently hydrogen or alkyl (in another embodiment each alkyl is C₁₋₃-alkyl); each R^{11a} is independently alkyl (in another embodiment each alkyl is C₁₋₃-alkyl), phenyl optionally substituted with alkoxy, or is heterocycloalkyl optionally substituted with one or two alkyl; each R¹⁶ is independently amino, alkylamino, dialkylamino, or cyclopropylamino; and all other groups are independently as defined in the Summary of the Invention for a Compound of Formula I or as defined in embodiment (1).

### Embodiments (D3)

In another embodiment, R² is according to Formula (a) where R³ is hydrogen, halo, alkyl, cycloalkylalkyl, or phenylalkyl optionally substituted with one or two R¹⁹; R^{3a} is hydrogen, alkyl, halo, optionally substituted heterocycloalkyl, or —NR¹¹R^{11a}; and R^{3b} is hydrogen, alkyl, hydroxyalkyl, cyanoalkyl, or alkyl substituted with one or two R¹⁶; and all other groups are independently as defined in the Summary of the Invention for a Compound of Formula I or as defined in embodiment (1).

### Embodiments (D3a)

In another embodiment, R² is according to Formula (a) where R³ is phenylalkyl optionally substituted with one or two R¹⁹; R^{3a} is alkyl; and R^{3b} is hydrogen, alkyl, hydroxyalkyl, or alkyl substituted with one R¹⁶; and all other groups are independently as defined in the Summary of the Invention for a Compound of Formula I or as defined in embodiment (1). In another embodiment, R² is according to Formula (a) where R³ is phenylalkyl optionally substituted with one or two R¹⁹; each R¹⁹ is independently halo, alkyl, haloalkyl, alkoxy, amino, alkylamino, or dialkylamino; R^{3a} is alkyl (in another embodiment alkyl is C₁₋₃-alkyl); and R^{3b} is hydrogen, alkyl, hydroxyalkyl, or alkyl substituted with one R¹⁶; R¹⁶ is amino, alkylamino, dialkylamino, cyclopropylamino, or —OC(O)CH₃; and all other groups are independently as defined in the Summary of the Invention for a Compound of Formula I or as defined in embodiment (1).

### Embodiments (D3b)

In another embodiment, R² is according to Formula (a) where R³ is phenylalkyl optionally substituted with one or two R¹⁹; R^{3a} and R^{3b} are alkyl; and all other groups are independently as defined in the Summary of the Invention for a Compound of Formula I or as defined in embodiment (1). In another embodiment, R² is according to Formula (a) where R³ is phenylalkyl optionally substituted with one or two R¹⁹; each R¹⁹ are independently halo, alkyl, haloalkyl, amino, alkylamino, dialkylamino, or alkoxy; R^{3a} and R^{3b} are alkyl (in another embodiment each alkyl is C₁₋₂-alkyl); and all other groups are independently as defined in the Summary of the Invention for a Compound of Formula I or as defined in embodiment (1). In another embodiment, R² is according to Formula (a) where R³ is phenylalkyl optionally substituted with one or two halo; R^{3a} and R^{3b} are alkyl (in another embodiment each alkyl is C₁₋₂-alkyl); and all other groups are independently as defined in the Summary of the Invention for a Compound of Formula I or as defined in embodiment (1). In another embodiment, R² is according to Formula (a) where R³ is phenylalkyl optionally substituted with one or two R¹⁹; each R¹⁹ are independently halo, alkyl, haloalkyl, amino, alkylamino, dialkylamino, or alkoxy; R^{3a} and R^{3b} are methyl; and all other groups are independently as defined in the Summary of the Invention for a Compound of Formula I or as defined in embodiment (1).

### Embodiments (D3c)

In another embodiment, R² is according to Formula (a) where R³ and R^{3a} are alkyl (in another embodiment each alkyl is C₁₋₂-alkyl); R^{3b} is hydrogen, alkyl, or alkyl substituted with one R¹⁶; and all other groups are independently as defined in the Summary of the Invention for a Compound of Formula I or as defined in embodiment (1). In another embodiment, R² is according to Formula (a) where R³ and R^{3a} are alkyl (in another embodiment alkyl is C₁₋₂-alkyl); R³¹ⁱ is hydrogen; and all other groups are independently as defined in the Summary of the Invention for a Compound of Formula I or as defined in embodiment (1). In another embodiment, R² is according to Formula (a) where R³, R^{3a}, and R^{3b} are alkyl (in another embodiment each alkyl is C₁₋₂-alkyl); and all other groups are independently as defined in the Summary of the Invention for a Compound of Formula I or as defined in embodiment (1). In another embodiment, R² is according to Formula (a) where R³ and R^{3a} are alkyl (in another embodiment each alkyl is C₁₋₂-alkyl); and R^{3b} is alkyl substituted with one R¹⁶; and all other groups are independently as defined in the Summary of the Invention for a Compound of Formula I or as defined in embodiment (1). In another embodiment, R² is according to Formula (a) where R³ and R^{3a} are alkyl (in another embodiment each alkyl is C₁₋₂-alkyl); and R^{3b} is alkyl substituted with one R¹⁶; R¹⁶ is amino, alkylamino, dialkylamino, or cycloalkylamino; and all other groups are independently as defined in the Summary of the Invention for a Compound of Formula I or as defined in embodiment (1).

### Embodiments (D3d)

In another embodiment, R² is according to Formula (a) where R³ is alkyl; R^{3a} and R^{3b} are hydrogen; and all other groups are independently as defined in the Summary of the Invention for a Compound of Formula I or as defined in embodiment (1). In another embodiment, R² is according to Formula (a) where R³ is C₁₋₂-alkyl; R^{3a} and R^{3b} are hydrogen; and all other groups are independently as defined in the Summary of the Invention for a Compound of Formula I or as defined in embodiment (1).

### Embodiments (D3e)

In another embodiment, R² is according to Formula (a) where R³ is phenylalkyl optionally substituted with one or two R¹⁹; R^{3a} is alkyl; and R^{3b} is hydrogen; and all other groups are independently as defined in the Summary of the Invention for a Compound of Formula I or as defined in embodiment (1). In another embodiment, R² is according to Formula (a) where R³ is phenylalkyl optionally substituted with one or two R¹⁹; each R¹⁹ is independently halo, alkyl, haloalkyl, amino, alkylamino, dialkylamino, or alkoxy; R^{3a} is alkyl; and R^{3b} is hydrogen; and all other groups are independently as defined in the Summary of the Invention for a Compound of Formula I or as defined in embodiment (1).

### Embodiments (D3f)

In another embodiment, R² is according to Formula (a) where R³ is phenylalkyl optionally substituted with one or two R¹⁹; R^{3a} is alkyl; and R^{3b} is alkyl substituted with one R¹⁶; and all other groups are independently as defined in the Summary of the Invention for a Compound of Formula I or as defined in embodiment (1). In another embodiment, R² is according to Formula (a) where R³ is phenylalkyl optionally substituted with one or two R¹⁹; each R¹⁹ is independently halo, alkyl, haloalkyl, amino, alkylamino, dialkylamino, or alkoxy; R^{3a} is alkyl (in another embodiment alkyl is C₁₋₂-alkyl); and R^{3b} is alkyl substituted with one R¹⁶; R¹⁶ is amino, alkylamino, dialkylamino, or cycloalkylamino; and all other groups are independently as defined in the Summary of the Invention for a Compound of Formula I or as defined in embodiment (1).

### Embodiments (D3g)

In another embodiment, R² is according to Formula (a) where R³ is alkyl or phenylalkyl optionally substituted with one or two R¹⁹; R^{3a} is alkyl; and R^{3b} is hydrogen, alkyl, or alkyl substituted with R¹⁶; and all other groups are independently as defined in the Summary of the Invention for a Compound of Formula I or as defined in embodiment (1). In another embodiment, R² is according to Formula (a) where R³ is alkyl (in another embodiment alkyl is C₁₋₂-alkyl) or phenylalkyl optionally substituted with one or two R¹⁹; R^{3a} is alkyl (in another embodiment alkyl is C₁₋₂-alkyl); and R^{3b} is hydrogen, alkyl (in another embodiment alkyl is C₁₋₂-alkyl), or alkyl substituted with R¹⁶; R¹⁶ is amino, alkylamino, dialkylamino, or cycloalkylamino; each R¹⁹ is independently halo, alkyl, haloalkyl, amino, alkylamino, dialkylamino, or alkoxy; and all other groups are independently as defined in the Summary of the Invention for a Compound of Formula I or as defined in embodiment (1).

### Embodiments (D3h)

In another embodiment, R² is according to Formula (a) where R³ is optionally substituted phenyloxy; R^{3a} is alkyl; and R^{3b} is hydrogen; and all other groups are independently as defined in the Summary of the Invention for a Compound of Formula I or as defined in embodiment (1). In another embodiment, R² is according to Formula (a) where R³ is phenyloxy optionally substituted with one or two groups which groups are independently halo, alkyl, haloalkyl, amino, alkylamino, dialkylamino, or alkoxy; R^{3a} is alkyl (in another embodiment alkyl is C₁₋₂-alkyl); and R^{3b} is hydrogen; and all other groups are independently as defined in the Summary of the Invention for a Compound of Formula I or as defined in embodiment (1). In another embodiment, R² is according to Formula (a) where R³ is phenyloxy; R^{3a} is alkyl (in another embodiment alkyl is C₁₋₂-alkyl); and R^{3b} is hydrogen; and all other groups are independently as defined in the Summary of the Invention for a Compound of Formula I or as defined in embodiment (1).

### Embodiments (D3i)

In another embodiment, R² is according to Formula (a) where R³ is optionally substituted cycloalkylalkyl; R^{3a} is alkyl; and R^{3b} is hydrogen or alkyl; and all other groups are independently as defined in the Summary of the Invention for a Compound of Formula I or as defined in embodiment (1). In another embodiment, R² is according to Formula (a) where R³ is cycloalkylalkyl; R^{3a} is alkyl (in another embodiment alkyl is C₁₋₂-alkyl); and R^{3b} is hydrogen or alkyl (in another embodiment alkyl is C₁₋₂-alkyl); and all other groups are independently as defined in the Summary of the Invention for a Compound of Formula I or as defined in embodiment (1).

### Embodiments (D3j)

In another embodiment, R² is according to Formula (a) where R³ is alkyl; R^{3a} is phenylalkyl optionally substituted with one or two R¹⁹; and R^{3b} is hydrogen; and all other groups are independently as defined in the Summary of the Invention for a Compound of Formula I or as defined in embodiment (1). In another embodiment, R² is according to Formula (a) where R³ is alkyl (in another embodiment alkyl is C₁₋₂-alkyl); R^{3a} is phenylalkyl optionally substituted with one or two R¹⁹; each R¹⁹ is independently halo, alkyl, haloalkyl, amino, alkylamino, dialkylamino, or alkoxy; and R^{3b} is hydrogen; and all other groups are independently as defined in the Summary of the Invention for a Compound of Formula I or as defined in embodiment (1). In another embodiment, R² is according to Formula (a) where R³ is alkyl (in another embodiment alkyl is C₁₋₂-alkyl); R^{3a} is phenylalkyl; and R^{3b} is hydrogen; and all other groups are independently as defined in the Summary of the Invention for a Compound of Formula I or as defined in embodiment (1).

### Embodiments (D3k)

In another embodiment, R² is according to Formula (a) where R³ is alkyl; R^{3a} is —NR¹¹R^{11a}; and R^{3b} is hydrogen or alkyl; and all other groups are independently as defined in the Summary of the Invention for a Compound of Formula I or as defined in embodiment (1). In another embodiment, R² is according to Formula (a) where R³ is alkyl (in another embodiment alkyl is C₁₋₂-alkyl); R^{3a} is NR¹¹R^{11a}; R^{3b} is hydrogen or alkyl (in another embodiment alkyl is C₁₋₂-alkyl); R¹¹ is hydrogen or alkyl (in another embodiment alkyl is C₁₋₂-alkyl); R^{11a} is alkyl, aminoalkyl, alkylaminoalkyl, dialkylaminoalkyl, optionally substituted heterocycloalkyl, optionally substituted heterocycloalkylalkyl, optionally substituted phenyl, or optionally substituted phenylalkyl; and all other groups are independently as defined in the Summary of the Invention for a Compound of Formula I or as defined in embodiment (1). In another embodiment, R² is according to Formula (a) where R³ is alkyl (in another embodiment alkyl is C₁₋₂-alkyl); R^{3a} is —NR¹¹R^{11a}; R^{3b} is hydrogen or alkyl (in another embodiment alkyl is C₁₋₂-alkyl); R¹¹ is hydrogen or alkyl (in another embodiment alkyl is C₁₋₂-alkyl); R^{11a} is alkyl, aminoalkyl, alkylaminoalkyl, dialkylaminoalkyl, heterocycloalkyl, heterocycloalkylalkyl (optionally substituted with one or two alkyl), phenylalkyl, phenyl (optionally substituted with one or two groups which are independently halo, alkyl, haloalkyl, amino, alkylamino, dialkylamino, or alkoxy); and all other groups are independently as defined in the Summary of the Invention for a Compound of Formula I or as defined in embodiment (1).

### Embodiments (D4)

In another embodiment, R² is according to Formula (a) where R^{3a} is alkyl (in another embodiment alkyl is C₁₋₂-alkyl), or —NR¹¹R^{11a}; R³ and R^{3b} are hydrogen; and all other groups are independently as defined in the Summary of the Invention for a Compound of Formula I or as defined in embodiment (1).

### Embodiments (D4a)

In another embodiment, R² is according to Formula (a) where R^{3a} is alkyl (in another embodiment alkyl is C₁₋₂-alkyl), and R³ and R^{3b} are hydrogen; and all other groups are independently as defined in the Summary of the Invention for a Compound of Formula I or as defined in embodiment (1).

### Embodiments (D4b)

In another embodiment, R² is according to Formula (a) where R^{3a} is —NR¹¹R^{11a}; R³ and R^{3b} are hydrogen; and all other groups are independently as defined in the Summary of the Invention for a Compound of Formula I or as defined in embodiment (1). In another embodiment, R² is according to Formula (a) where R^{3a} is —NR¹¹R^{11a}; R³ and R^{3b} are hydrogen; R¹¹ is hydrogen or alkyl; R^{11a} is optionally substituted phenyl; and all other groups are independently as defined in the Summary of the Invention for a Compound of Formula I or as defined in embodiment (1). In another embodiment, R² is according to Formula (a) where R^{3a} is —NR¹¹R^{11a}; R³ and R^{3b} are hydrogen; R¹¹ is hydrogen or alkyl (in another embodiment alkyl is C₁₋₂-alkyl); R^{11a} is phenyl optionally substituted with one or two groups which groups are independently halo, alkyl, haloalkyl, amino, alkylamino, dialkylamino, or alkoxy; and all other groups are independently as defined in the Summary of the Invention for a Compound of Formula I or as defined in embodiment (1).

### Embodiments (D5)

In another embodiment, R² is according to Formula (a) where R³ and R^{3a} are hydrogen; R^{3b} is —NR¹¹R^{11a}; and all other groups are independently as defined in the Summary of the Invention for a Compound of Formula I or as defined in embodiment (1). In another embodiment, R² is according to Formula (a) where R³ and R^{3a} are hydrogen; R^{3b} is —NR¹¹R^{11a}; R¹¹ is hydrogen or alkyl (in another embodiment alkyl is C₁₋₂-alkyl); R^{11a} is optionally substituted phenyl; and all other groups are independently as defined in the Summary of the Invention for a Compound of Formula I or as defined in embodiment (1). In another embodiment, R² is according to Formula (a) where R³ and R^{3a} are hydrogen; R^{3b} is —NR¹¹R^{11a}; and all other groups are independently as defined in the Summary of the Invention for a Compound of Formula I or as defined in embodiment (1). In another embodiment, R² is according to Formula (a) where R³ and R^{3a} are hydrogen; R^{3b} is —NR¹¹R^{11a}; R¹¹ is hydrogen or alkyl (in another embodiment alkyl is C₁₋₂-alkyl); R^{11a} is hydrogen, alkyl (in another embodiment alkyl is C₁₋₂-alkyl), or optionally substituted phenyl; and all other groups are independently as defined in the Summary of the Invention for a Compound of Formula I or as defined in embodiment (1). In another embodiment, R² is according to Formula (a) where R³ and R^{3a} are hydrogen; R^{3b} is —NR¹¹R^{11a}; R¹¹ is hydrogen or alkyl (in another embodiment alkyl is C₁₋₂-alkyl); R^{11a} is hydrogen, alkyl (in another embodiment alkyl is C₁₋₂-alkyl), or phenyl optionally substituted with one or two groups which groups are independently halo, alkyl, haloalkyl, amino, alkylamino, dialkylamino, or alkoxy; and all other groups are independently as defined in the Summary of the Invention for a Compound of Formula I or as defined in embodiment (1). In another embodiment, R² is according to Formula (a) where R³ and R^{3a} are hydrogen; R^{3b} is —NR¹¹R^{11a}; R¹¹ is hydrogen or alkyl (in another embodiment alkyl is C₁₋₂-alkyl); R^{11a} is hydrogen, alkyl (in another embodiment alkyl is C₁₋₂-alkyl), or phenyl; and all other groups are independently as defined in the Summary of the Invention for a Compound of Formula I or as defined in embodiment (1).

### Embodiments (D6)

In another embodiment, R² is according to Formula (a) where R³ is hydrogen; R^{3a} is alkyl (in another embodiment alkyl is C₁₋₂-alkyl) or —NR¹¹R^{11a}; R^{3b} is hydrogen or alkyl (in another embodiment alkyl is C₁₋₂-alkyl); and all other groups are independently as defined in the Summary of the Invention for a Compound of Formula I or as defined in embodiment (1).

### Embodiments (D6a)

In another embodiment, R² is according to Formula (a) where R³ is hydrogen; R^{3a} is alkyl (in another embodiment alkyl is C₁₋₂-alkyl); R^{3b} is hydrogen; and all other groups are independently as defined in the Summary of the Invention for a Compound of Formula I or as defined in embodiment (1).

### Embodiments (D6b)

In another embodiment, R² is according to Formula (a) where R^{3a}—NR¹¹R^{11a}; R³ and R^{3b} are hydrogen; and all other groups are independently as defined in the Summary of the Invention for a Compound of Formula I or as defined in embodiment (1). In another embodiment, R² is according to Formula (a) where R^{3a} is —NR¹¹R^{11a}; R³ and R^{3b} are hydrogen; R¹¹ is hydrogen or alkyl (in another embodiment alkyl is C₁₋₂-alkyl); R^{11a} is hydrogen, alkyl (in another embodiment alkyl is C₁₋₂-alkyl), or optionally substituted phenyl; and all other groups are independently as defined in the Summary of the Invention for a Compound of Formula I or as defined in embodiment (1). In another embodiment, R² is according to Formula (a) where R^{3a} is —NR¹¹R^{11a}; R³ and R^{3b} are hydrogen; R¹¹ is hydrogen or alkyl (in another embodiment alkyl is C₁₋₂-alkyl); R^{11a} is hydrogen, alkyl (in another embodiment alkyl is C₁₋₂-alkyl), or phenyl optionally substituted with one or two groups which groups are independently halo, alkyl, haloalkyl, amino, alkylamino, dialkylamino, or alkoxy; and all other groups are independently as defined in the Summary of the Invention for a Compound of Formula I or as defined in embodiment (1). In another embodiment, R² is according to Formula (a) where R^{3a} is —NR¹¹R^{11a}; R³ and R^{3b} are hydrogen; R¹¹ is hydrogen or alkyl (in another embodiment alkyl is C₁₋₂-alkyl); R^{11a} is hydrogen, alkyl (in another embodiment each alkyl is C₁₋₂-alkyl), or phenyl optionally substituted with one alkoxy; and all other groups are independently as defined in the Summary of the Invention for a Compound of Formula I or as defined in embodiment (1).

### Embodiments (D6c)

In another embodiment, R² is according to Formula (a) where R³, R^{3a}, and R^{3b} are hydrogen; and all other groups are independently as defined in the Summary of the Invention for a Compound of Formula I or as defined in embodiment (1).

### Embodiments (D6d)

In another embodiment, R² is pyrimidin-2-yl, pyrimidin-4-yl, 5-(phenylmethyl)-6-methyl-pyrimidin-4-yl, 6-(phenylmethyl)-5-methyl-pyrimidin-4-yl, 5-(1-phenylethyl)-6-methyl-pyrimidin-4-yl, 2,6-dimethyl-5-(phenylmethyl)-pyrimidin-4-yl, 5-(phenylmethyl)-6-ethyl-pyrimidin-4-yl, 2-methyl-pyrimidin-4-yl, 5-methyl-pyrimidin-4-yl, 6-methyl-pyrimidin-4-yl, 5,6-dimethyl-pyrimidin-4-yl, 6-isopropyl-pyrimidin-4-yl, 5-methyl-6-ethyl-pyrimidin-4-yl, 5-isopropyl-6-methyl-pyrimidin-4-yl, 5-isoamyl-6-methyl-pyrimidin-4-yl, 5-ethyl-6-isopropyl-pyrimidin-4-yl, 5-methyl-6-isopropyl-pyrimidin-4-yl, 5-(phenylmethyl)-6-chloro-pyrimidin-4-yl, 5-(phenylmethyl)-pyrimidin-4-yl, 5-phenyloxy-6-methyl-pyrimidin-4-yl, 5-(cyclopropylmethyl)-6-methyl-pyrimidin-4-yl, 2-amino-pyrimidin-4-yl, 5-(2-chloro-phenylmethyl)-6-methyl-pyrimidin-4-yl, 5-(3-chloro-phenylmethyl)-6-methyl-pyrimidin-4-yl, 5-(4-chloro-phenylmethyl)-6-methyl-pyrimidin-4-yl, 5-(2-fluoro-phenylmethyl)-6-methyl-pyrimidin-4-yl, 5-(3-fluoro-phenylmethyl)-6-methyl-pyrimidin-4-yl, 5-(4-fluoro-phenylmethyl)-6-methyl-pyrimidin-4-yl, 5-(3,4-difluoro-phenylmethyl)-6-methyl-pyrimidin-4-yl, 5-(3,5-difluoro-phenylmethyl)-6-methyl-pyrimidin-4-yl, 5-(3-chloro-5-fluoro-phenylmethyl)-6-methyl-pyrimidin-4-yl, 5-(1-(3-fluorophenyl)-ethyl)-6-methyl-pyrimidin-4-yl, 2,6-dimethyl-5-(4-fluoro-phenylmethyl)-pyrimidin-4-yl, 5-(2-methyl-phenylmethyl)-6-methyl-pyrimidin-4-yl, 5-(3-methyl-phenylmethyl)-6-methyl-pyrimidin-4-yl, 5-(4-methyl-phenylmethyl)-6-methyl-pyrimidin-4-yl, 5-(4-chloro-3-(dimethylamino)-phenylmethyl)-6-methyl-pyrimidin-4-yl, 5-(2-methoxy-phenylmethyl)-6-methyl-pyrimidin-4-yl, 5-(3-methoxy-phenylmethyl)-6-methyl-pyrimidin-4-yl, 5-(4-methoxy-phenylmethyl)-6-methyl-pyrimidin-4-yl, 2-(phenylamino)-pyrimidin-4-yl, 6-(phenylamino)-pyrimidin-4-yl, 6-(4-methoxy-phenylamino)-pyrimidin-4-yl, 5-methyl-6-(phenylamino)-pyrimidin-4-yl, 542-trifluoromethyl-phenylmethyl)-6-methyl-pyrimidin-4-yl, 5-(3-trifluoromethyl-phenylmethyl)-6-methyl-pyrimidin-4-yl, 5-(4-trifluoromethyl-phenylmethyl)-6-methyl-pyrimidin-4-yl, or 5-phenylmethyl-6-trifluoromethyl-pyrimidin-4-yl; and all other groups are independently as defined in the Summary of the Invention for a Compound of Formula I or as defined in embodiment (1).

### Embodiments (D7)

In another embodiment, R² is pyridinyl substituted with R³, R^{3a}, R^{3b}, and R^{3c}; where R³, R^{3a}, R^{3b}, and R^{3c} and all other groups are independently as defined in the Summary of the Invention for a Compound of Formula I or as defined in embodiment (1).

### Embodiments (D7a)

In another embodiment, R² is pyridinyl substituted with R³, R^{3a}, R^{3b}, and R^{3c} where R³, R^{3a}, R^{3b}, and R^{3c} are independently hydrogen, alkyl, or phenylalkyl optionally substituted with one or two R¹⁹; and all other groups are independently as defined in the Summary of the Invention for a Compound of Formula I or as defined in embodiment (1). In another embodiment, R² is pyridinyl substituted with R³, R^{3a}, R^{3b}, and R^{3c}; where R³, R^{3a}, R^{3b}, and R^{3c} are independently hydrogen, alkyl, phenylalkyl, or phenylalkyl substituted with one or two halo; and all other groups are independently as defined in the Summary of the Invention for a Compound of Formula I or as defined in embodiment (1).

### Embodiments (D7b)

In another embodiment, R² is pyridinyl substituted with R³, R^{3a}, R^{3b}, and R^{3c}; where R³ is alkyl (in another embodiment alkyl is C₁₋₂-alkyl); R³, R^{3a}, R^{3b}, and R^{3c} are hydrogen; and all other groups are independently as defined in the Summary of the Invention for a Compound of Formula I or as defined in embodiment (1).

### Embodiments (D7c)

In another embodiment, R² is pyridin-2-yl, pyridin-3-yl, pyridin-4-yl, 2-amino-pyridin-4-yl, 3-methyl-pyridin-2-yl, 2-methyl-3-(phenylmethyl)-pyridin-4-yl, 3-(2-fluoro-phenylmethyl)-2-methyl-pyridin-4-yl, 3-(3-fluoro-phenylmethyl)-2-methyl-pyridin-4-yl, or 3-(4-fluoro-phenylmethyl)-2-methyl-pyridin-4-yl; and all other groups are independently as defined in the Summary of the Invention for a Compound of Formula I or as defined in embodiment (1).

### Embodiments (D7d)

In another embodiment, R² is according to Formula (b)

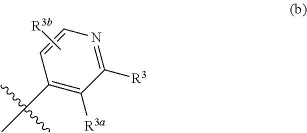

where R³, R^{3a}, and R^{3b} are independently as defined in the Summary of the Invention for a Compound of Formula I or as defined in embodiment (1).

### Embodiments (E)

In another embodiment, R² is a 10-membered heteroaryl substituted with R³, R^{3a}, R^{3b}, R^{3c}, and R^{3d}; where R³, R^{3a}, R^{3b}, R^{3c}, and R^{3d} and all other groups are independently as defined in the Summary of the Invention for a Compound of Formula I or as defined in embodiment (1). In another embodiment, R² is a 10-membered heteroaryl and the 10-membered heteroaryl is quinazolin-2-yl, quinazolin-4-yl, quinazolin-5-yl, quinazolin-6-yl, quinazolin-7-yl, quinazolin-8-yl, pyrido[3,2-d]pyrimidin-4-yl, pyrido[4,3-d]pyrimidin-4-yl, pyrido[3,4-d]pyrimidin-4-yl, pyrido[2,3-d]pyrimidin-4-yl, 6,7-dihydro-5H-cyclopenta[d]pyrimidin-4-yl, 5,6,7,8-tetrahydroquinazolin-4-yl, quinolin-2-yl, quinolin-3-yl, quinolin-4-yl, quinolin-5-yl, quinolin-6-yl, quinolin-7-yl, quinolin-8-yl, isoquinolin-1-yl, isoquinolin-3-yl, isoquinolin-4-yl, isoquinolin-5-yl, isoquinolin-6-yl, isoquinolin-7-yl, isoquinolin-8-yl, thieno[2,3-d]pyrimidin-4-yl, 7H-pyrrolo[2,3-d]pyrimidin-4-yl, 1H-pyrrolo[2,3-b]pyridin-4-yl, 1H-pyrrolo[3,2-c]pyridin-4-yl, thieno[2,3-b]pyridin-4-yl, thieno[3,2-c]pyridin-4-yl, 5,7-dihydrothieno[3,4-d]pyrimidin-4-yl, 5,6,7,8-tetrahydropyrido[3,4-d]pyrimidin-4-yl, 5,6,7,8-tetrahydropyrido[4,3-d]pyrimidin-4-yl, 5,6,7,8-tetrahydropyrido[2,3-d]pyrimidin-4-yl, 5,6,7,8-tetrahydropyrido[3,2-d]pyrimidin-4-yl, 6,7-dihydro-5H-pyrrolo[3,4-d]pyrimidin-4-yl, 6,7-dihydro-5H-pyrrolo[3,2-d]pyrimidin-4-yl, 6,7-dihydro-5H-pyrrolo[2,3-d]pyrimidin-4-yl, or 5,6-dihydroquinazolinyl where R² is substituted with R³, R^{3a}, R^{3b}, R^{3c}, and R^{3d}; where R³, R^{3a}, R^{3b}, R^{3c}, and R^{3d} and all other groups are independently as defined in the Summary of the Invention for a Compound of Formula I or as defined in embodiment (1).

### Embodiments (E1)

In another embodiment, R² is quinazolin-2-yl, quinazolin-4-yl, quinazolin-5-yl, quinazolin-6-yl, quinazolin-7-yl, or quinazolin-8-yl, where R² is substituted with R³, R^{3a}, R^{3b}, R^{3c}, and R^{3d}; where R³, R^{3a}, R^{3b}, R^{3c}, and R^{3d} and all other groups are independently as defined in the Summary of the Invention for a Compound of Formula I or as defined in embodiment (1).

### Embodiments (E2)

In another embodiment, R² is quinazolin-4-yl substituted with R³, R^{3a}, R^{3b}, R^{3c}, and R^{3d}; where R³, R^{3a}, R^{3b}, R^{3c}, and R^{3d} and all other groups are independently as defined in the Summary of the Invention for a Compound of Formula I or as defined in embodiment (1). In another embodiment, R² is quinazolin-4-yl substituted with R³, R^{3a}, R^{3b}, R^{3c}, and R^{3d}; where R³, R^{3a}, R^{3b}, R^{3c}, and R^{3d} are independently hydrogen, halo, alkyl, haloalkyl, alkoxycarbonyl, optionally substituted phenyl, —S(O)₂R²⁰, —NR¹¹R^{11a}, or —OR^{11a}; and all other groups are independently as defined in the Summary of the Invention for a Compound of Formula I or as defined in embodiment (1).

### Embodiments (E2a)

In another embodiment, R² is quinazolin-4-yl substituted with R³, R^{3a}, R^{3b}, R^{3c}, and R^{3d}; R^{3c} and R^{3d} are hydrogen and R³, R^{3a}, and R^{3b} are independently cyano, alkyl, alkenyl, halo, haloalkyl, hydroxyalkyl, alkoxyalkyl, —SR¹², —S(O)₂R²⁰, —C(O)OR⁴, halocarbonyl, —NR¹¹R^{11a}, —OR^{11a}, optionally substituted phenyl, optionally substituted phenylalkyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, or alkyl substituted with one or two R¹⁶; and all other groups are independently as defined in the Summary of the Invention for a Compound of Formula I or as defined in embodiment (1). In another embodiment, R² is quinazolin-4-yl substituted with R³, R^{3a}, R^{3b}, R^{3c}, and R^{3d}; R^{3a} and R^{3d} are hydrogen and R³, R^{3a}, and R^{3b} are independently alkyl, halo, or —OR^{11a}; and all other groups are independently as defined in the Summary of the Invention for a Compound of Formula I or as defined in embodiment (1). In another embodiment, R² is quinazolin-4-yl substituted with R³, R^{3a}, R^{3b}, R^{3c}, and R^{3d}; R^{3c} and R^{3d} are hydrogen and R³, R^{3a}, and R^{3b} are independently alkyl, halo, or —OR^{11a}; R^{11a} is hydrogen, alkyl, or alkoxyalkyl; and all other groups are independently as defined in the Summary of the Invention for a Compound of Formula I or as defined in embodiment (1).

### Embodiments (E2b)

In another embodiment, R² is quinazolin-4-yl substituted with R³, R^{3a}, R^{3b}, R^{3c}, and R^{3d}; R^{3b}, R^{3c}, and R^{3d} are hydrogen, and R³ and R^{3a} are independently cyano, alkyl, alkenyl, halo, haloalkyl, hydroxyalkyl, alkoxyalkyl, —SR¹², —S(O)₂R²⁰, —C(O)OR⁴, halocarbonyl, —NR¹¹R^{11a}, —OR^{11a}, optionally substituted phenyl, optionally substituted phenylalkyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, or alkyl substituted with one or two R¹⁶; and all other groups are independently as defined in the Summary of the Invention for a Compound of Formula I or as defined in embodiment (1). In another embodiment, R² is quinazolin-4-yl substituted with R³, R^{3a}, R^{3b}, R^{3c}, and R^{3d}; R^{3b}, R^{3c}, and R^{3d} are hydrogen, and R³ and R^{3a} are independently alkyl, halo, —S(O)₂R²⁰, —OR^{11a}, or alkyl substituted with one R¹⁶; and all other groups are independently as defined in the Summary of the Invention for a Compound of Formula I or as defined in embodiment (1). In another embodiment, R² is quinazolin-4-yl substituted with R³, R^{3a}, R^{3b}, R^{3c}, and R^{3d}; R^{3b}, R^{3c}, and R^{3d} are hydrogen, and R³ and R^{3a} are independently alkyl, halo, —S(O)₂R²⁰, —OR^{11a}, or alkyl substituted with one R¹⁶; R^{11a} is hydrogen, alkyl, aminoalkyl, alkylaminoalkyl, dialkylaminoalkyl, phenyl, cycloalkylalkyl, phenylalkyl, or heteroaryl; R¹⁶ is amino, alkylamino, dialkylamino, or cycloalkylamino; R²⁰ is alkyl; and all other groups are independently as defined in the Summary of the Invention for a Compound of Formula I or as defined in embodiment (1). In another embodiment, R² is quinazolin-4-yl substituted with R³, R^{3a}, R^{3b}, R^{3c}, and R^{3d}; R^{3b}, R^{3c}, and R^{3d} are hydrogen, and R³ is —OR^{11a} and R^{3a} is hydrogen, alkyl (in another embodiment alkyl is C₁₋₂-alkyl), or alkyl substituted with one R¹⁶; and all other groups are independently as defined in the Summary of the Invention for a Compound of Formula I or as defined in embodiment (1). In another embodiment, R² is quinazolin-4-yl substituted with R³, R^{3a}, R^{3b}, R^{3c}, and R^{3d}; R^{3b}, R^{3c}, and R^{3d} are hydrogen, and R³ is —OR^{1a} and R^{3a} is hydrogen, alkyl, or alkyl substituted with one R¹⁶; R^{11a} is hydrogen or alkyl; R¹⁶ is amino, alkylamino, dialkylamino, or cycloalkylamino; and all other groups are independently as defined in the Summary of the Invention for a Compound of Formula I or as defined in embodiment (1).

### Embodiments (E2c)

In another embodiment, R² is quinazolin-4-yl substituted with R³, R^{3a}, R^{3b}, R^{3c}, and R^{3d}; R^{3a}, R^{3b}, R^{3c}, and R^{3d} are hydrogen and R³ is cyano, alkyl, alkenyl, halo, haloalkyl, hydroxyalkyl, alkoxyalkyl, —SR¹², —S(O)₂R²⁰, —C(O)OR⁴, halocarbonyl, —NR¹¹R^{11a}, —OR^{11a}, optionally substituted phenyl, optionally substituted phenylalkyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, or alkyl substituted with one or two R¹⁶; and all other groups are independently as defined in the Summary of the Invention for a Compound of Formula I or as defined in embodiment (1). In another embodiment, R² is quinazolin-4-yl substituted with R³, R^{3a}, R^{3b}, R^{3c}, and R^{3d}; R^{3a}, R^{3b}, R^{3c}, and R^{3d} are hydrogen and R³ is alkyl, halo, haloalkyl, alkylsulfonyl, optionally substituted phenyl, carboxy, alkoxycarbonyl, —NR¹¹R^{11a}, alkyl substituted with one R¹⁶, or —OR^{11a}; and all other groups are independently as defined in the Summary of the Invention for a Compound of Formula I or as defined in embodiment (1). In another embodiment, R² is quinazolin-4-yl substituted with R³, R^{3a}, R^{3b}, R^{3c}, and R^{3d}; R^{3a}, R^{3b}, R^{3c}, and R^{3d} are hydrogen and R³ is alkyl, halo, haloalkyl, alkylsulfonyl, phenyl, carboxy, alkoxycarbonyl, —NR¹¹R^{11a}, alkyl substituted with one R¹⁶, or —OR^{11a}; R¹¹ is hydrogen or alkyl; R^{11a} is hydrogen, alkyl, alkoxyalkyl, cyanoalkyl, or optionally substituted phenylalkyl; R¹⁶ is amino, alkylamino, dialkylamino, or cycloalkylamino; and all other groups are independently as defined in the Summary of the Invention for a Compound of Formula I or as defined in embodiment (1). In another embodiment, R² is quinazolin-4-yl substituted with R³, R^{3a}, R^{3b}, R^{3c}, and R^{3d}; R^{3a}, R^{3b}, R^{3c}, and R^{3d} are hydrogen and R³ is methyl, ethyl, n-propyl, isopropyl, n-butyl, sec-butyl, isoamyl, bromo, chloro, fluoro, iodo, trifluoromethyl, methylsulfonyl, phenyl, methoxycarbonyl, ethoxycarbonyl, amino, methylamino, ethylamino, n-propylamino, isopropylamino, dimethylamino, diethylamino, N-methyl-N-ethylamino, hydroxy, methoxy, ethyloxy, n-propoxy, isopropoxy, n-butyloxy, sec-butyloxy, isoamyloxy, 2-amino-ethyloxy, 2-(methylamino)-ethyloxy, 2-(dimethylamino)-ethyloxy, 3-amino-propyloxy, 3-(methylamino)-propyloxy, 3-(dimethylamino)-propyloxy, 2-methoxy-ethyloxy, cyanomethyloxy, and benzyloxy; and all other groups are independently as defined in the Summary of the Invention for a Compound of Formula I or as defined in embodiment (1).

### Embodiments (E2d)

In another embodiment, R² is quinazolin-4-yl, pyrido[3,2-d]pyrimidin-4-yl, pyrido[4,3-d]pyrimidin-4-yl, pyrido[3,4-d]pyrimidin-4-yl, pyrido[2,3-d]pyrimidin-4-yl, 2-methyl-quinazolin-4-yl, 6-methyl-quinazolin-4-yl, 7-methyl-quinazolin-4-yl, 8-methyl-quinazolin-4-yl, 2-ethyl-quinazolin-4-yl, 2-phenyl-quinazolin-4-yl, 7-(quinolin-2-ylmethyloxy)-8-methoxy-quinazolin-4-yl, 7-(2-dimethylamino-ethyloxy)-8-methoxy-quinazolin-4-yl, 6-(3-dimethylamino-propyloxy)-8-methoxy-quinazolin-4-yl, 7-(cyclopropylmethyloxy)-8-methoxy-quinazolin-4-yl, 6-(cyanomethyloxy)-quinazolin-4-yl, 6-methoxy-quinazolin-4-yl, 7-methoxy-quinazolin-4-yl, 8-methoxy-quinazolin-4-yl, 6-ethoxy-quinazolin-4-yl, 6-(n-propoxy)-quinazolin-4-yl, 6,7-dimethoxy-quinazolin-4-yl, 7,8-dimethoxy-quinazolin-4-yl, 7-isoamyloxy-8-methoxy-quinazolin-4-yl, 5-bromo-quinazolin-4-yl, 6-bromo-quinazolin-4-yl, 7-bromo-quinazolin-4-yl, 8-bromo-quinazolin-4-yl, 5-chloro-quinazolin-4-yl, 6-chloro-quinazolin-4-yl, 7-chloro-quinazolin-4-yl, 8-chloro-quinazolin-4-yl, 5-fluoro-quinazolin-4-yl, 6-fluoro-quinazolin-4-yl, 7-fluoro-quinazolin-4-yl, 8-fluoro-quinazolin-4-yl, 5-iodo-quinazolin-4-yl, 6-iodo-quinazolin-4-yl, 7-iodo-quinazolin-4-yl, 8-iodo-quinazolin-4-yl, 6-bromo-7-chloro-quinazolin-4-yl, 6-iodo-7-chloro-quinazolin-4-yl, 6,8-dichloro-quinazolin-4-yl, 6,7-difluoro-quinazolin-4-yl, 6,8-dibromo-quinazolin-4-yl, 2-methyl-7-methoxy-quinazolin-4-yl, 2-ethyl-7-methoxy-quinazolin-4-yl, 2-methyl-6,7-dimethoxy-quinazolin-4-yl, 6-iodo-7-methoxy-quinazolin-4-yl, 6-chloro-7-methoxy-quinazolin-4-yl, 2-chloro-6-methoxy-quinazolin-4-yl, 6-bromo-7-methoxy-quinazolin-4-yl, 7-bromo-8-methoxy-quinazolin-4-yl, 7-bromo-6-methoxy-quinazolin-4-yl, 6-chloro-7,8-dimethoxy-quinazolin-4-yl, 6,7,8-trimethoxy-quinazolin-4-yl, 6-(2-methoxy-ethyloxy)-quinazolin-4-yl, 6-(benzyoxy)-quinazolin-4-yl, 6-hydroxy-quinazolin-4-yl, 7-(benzyoxy)-8-methoxy-quinazolin-4-yl, 7-hydroxy-8-methoxy-quinazolin-4-yl, 7-(benzyoxy)-6-methoxy-quinazolin-4-yl, 7-hydroxy-6-methoxy-quinazolin-4-yl, 6-iodo-8-methyl-quinazolin-4-yl, 6-methyl-8-bromo-quinazolin-4-yl, 2-ethoxycarbonyl-quinazolin-4-yl, 2-methylamino-quinazolin-4-yl, 2-ethylamino-quinazolin-4-yl, 2-(diethylamino)-quinazolin-4-yl, 2-(trifluoromethyl)-quinazolin-4-yl, 7-(trifluoromethyl)-quinazolin-4-yl, 8-(trifluoromethyl)-quinazolin-4-yl, 6-methylsulfonyl-quinazolin-4-yl, 7-methylsulfonyl-quinazolin-4-yl, quinazolin-4-yl, quinazolin-4-yl, or quinazolin-4-yl; and all other groups are independently as defined in the Summary of the Invention for a Compound of Formula I or as defined in embodiment (1).

### Embodiments (E2e)

In another embodiment, R² is pyrido[3,2-d]pyrimidin-4-yl; and all other groups are independently as defined in the Summary of the Invention for a Compound of Formula I or as defined in embodiment (1).

### Embodiments (E3)

In another embodiment, R² is 5,6,7,8-tetrahydroquinazolin-4-yl, 6,7-dihydro-5H-cyclopenta[d]pyrimidin-4-yl, 6,7,8,9-tetrahydro-5H-cyclohepta[d]pyrimidin-4-yl, 5,6-dihydroquinazolin-4-yl, 7′,8′-dihydro-5′H-spiro[cyclopropane-1,6′-quinazoline]-4′-yl, or 6′,8′-dihydro-5′H-spiro[cyclopropane-1,7′-quinazoline]-4′-yl where R² is substituted with R³, R^{3a}, R^{3b}, R^{3c}, and R^{3d}; where R³, R^{3a}, R^{3b}, R^{3c}, and R^{3d} and all other groups are independently as defined in the Summary of the Invention for a Compound of Formula I or as defined in embodiment (1). In another embodiment, R² is 5,6,7,8-tetrahydroquinazolin-4-yl, 6,7-dihydro-5H-cyclopenta[d]pyrimidin-4-yl, 6,7,8,9-tetrahydro-5H-cyclohepta[d]pyrimidin-4-yl, 5,6-dihydroquinazolin-4-yl, 7′,8′-dihydro-5′H-spiro[cyclopropane-1,6′-quinazoline]-4′-yl, or 6′,8′-dihydro-5′H-spiro[cyclopropane-1,7′-quinazoline]-4′-yl where R² is substituted with R³, R^{3a}, R^{3b}, R^{3c}, and R^{3d}; where R³, R^{3a}, R^{3b}, R^{3c}, and R^{3d} are hydrogen; and all other groups are independently as defined in the Summary of the Invention for a Compound of Formula I or as defined in embodiment (1).

### Embodiments (E3a)

In another embodiment, R² is 5,6,7,8-tetrahydroquinazolin-4-yl, 6,7-dihydro-5H-cyclopenta[d]pyrimidin-4-yl, 6,7,8,9-tetrahydro-5H-cyclohepta[c]pyrimidin-4-yl, 5,6-dihydroquinazolin-4-yl, or 7′,8′-dihydro-5′H-spiro[cyclopropane-1,6′-quinazoline]-4′-yl, where R² is substituted with R³, R^{3a}, R^{3b}, R^{3c}, and R^{3d}; where R³, R^{3a}, R^{3b}, R^{3c}, and R^{3d} are independently hydrogen, alkyl, alkenyl, halo, haloalkyl, hydroxyalkyl, cyanoalkyl, —SR¹², optionally substituted phenyl, —OR^{11a}, alkyl substituted with one R¹⁶, optionally substituted heterocycloalkyl, optionally substituted heterocycloalkylalkyl, or optionally substituted heteroaryl; and all other groups are independently as defined in the Summary of the Invention for a Compound of Formula I or as defined in embodiment (1). In another embodiment, R² is 5,6,7,8-tetrahydroquinazolin-4-yl, 6,7-dihydro-5H-cyclopenta[d]pyrimidin-4-yl, 6,7,8,9-tetrahydro-5H-cyclohepta[d]pyrimidin-4-yl, 5,6-dihydroquinazolin-4-yl, or 7′,8′-dihydro-5′H-spiro[cyclopropane-1,6′-quinazoline]-4′-yl, where R² is substituted with R³, R^{3a}, R^{3b}, R^{3c}, and R^{3d}; where R³, R^{3a}, R^{3b}, R^{3c}, and R^{3d} are independently hydrogen, alkyl, alkenyl, halo, haloalkyl, hydroxyalkyl, cyanoalkyl, —SR¹², phenyl, —OR^{11a}, alkyl substituted with one R¹⁶, heterocycloalkyl (optionally substituted with alkoxycarbonyl, phenylalkyloxycarbonyl, or alkyl), heterocycloalkylalkyl (optionally substituted with one or two halo), or heteroaryl; R¹² is alkyl or phenylalkyl; R¹⁶ is NR¹¹R^{11a}, —NR¹⁵S(O)R^{15a}, —OR¹⁸, or —OC(O)R¹⁷; R¹¹ is hydrogen or alkyl; each R^{11a} is independently hydrogen, alkyl, haloalkyl, alkoxyalkyl, carboxyalkyl, cycloalkyl, or cycloalkylalkyl; and all other groups are independently as defined in the Summary of the Invention for a Compound of Formula I or as defined in embodiment (1).

### Embodiments (E3b)

In another embodiment, R² is 5,6,7,8-tetrahydroquinazolin-4-yl, 6,7-dihydro-5H-cyclopenta[d]pyrimidin-4-yl, 6,7,8,9-tetrahydro-5H-cyclohepta[d]pyrimidin-4-yl, 5,6-dihydroquinazolin-4-yl, or 7′,8′-dihydro-5′H-spiro[cyclopropane-1,6′-quinazoline]-4′-yl, where R² is substituted with R³, R^{3a}, R^{3b}, R^{3c}, and R^{3d}; where R^{3a}, R^{3b}, R^{3c}, and R^{3d} are hydrogen, and R³ and all other groups are independently as defined in the Summary of the Invention for a Compound of Formula I or as defined in embodiment (1). In another embodiment, R² is 5,6,7,8-tetrahydroquinazolin-4-yl, 6,7-dihydro-5H-cyclopenta[d]pyrimidin-4-yl, 6,7,8,9-tetrahydro-5H-cyclohepta[d]pyrimidin-4-yl, 5,6-dihydroquinazolin-4-yl, or 7′,8′-dihydro-5′H-spiro[cyclopropane-1,6′-quinazoline]-4′-yl, where R² is substituted with R³, R^{3a}, R^{3b}, R^{3c}, and R^{3d}; where R^{3a}, R^{3b}, R^{3c}, and R^{3d} are hydrogen, and R³ is alkyl, alkenyl, hydroxyalkyl, alkoxyalkyl, haloalkyl, optionally substituted phenyl, alkyl substituted with one R¹⁶, or —SR¹²; and all other groups are independently as defined in the Summary of the Invention for a Compound of Formula I or as defined in embodiment (1). In another embodiment, R² is 5,6,7,8-tetrahydroquinazolin-4-yl, 6,7-dihydro-5H-cyclopenta[d]pyrimidin-4-yl, 6,7,8,9-tetrahydro-5H-cyclohepta[d]pyrimidin-4-yl, 5,6-dihydroquinazolin-4-yl, 7′,8′-dihydro-5′H-spiro[cyclopropane-1,6′-quinazoline]-4′-yl, where R² is substituted with R³, R^{3a}, R^{3b}, R^{3c}, and R^{3d}; where R^{3a}, R^{3b}, R^{3c}, and R^{3d} are hydrogen, and R³ is alkyl, alkenyl, hydroxyalkyl, alkoxyalkyl, haloalkyl, phenyl, alkyl substituted with one R¹⁶, or —SR¹²; R¹² is alkyl or optionally substituted phenylalkyl; and all other groups are independently as defined in the Summary of the Invention for a Compound of Formula I or as defined in embodiment (1).

### Embodiments (E3c)

In another embodiment, R² is 5,6,7,8-tetrahydroquinazolin-4-yl, 6,7-dihydro-5H-cyclopenta[d]pyrimidin-4-yl, 6,7,8,9-tetrahydro-5H-cyclohepta[d]pyrimidin-4-yl, 5,6-dihydroquinazolin-4-yl, or 7′,8′-dihydro-5′H-spiro[cyclopropane-1,6′-quinazoline]-4′-yl, where R² is substituted with R³, R^{3a}, R^{3b}, R^{3c}, and R^{3d}; where R^{3b}, R^{3c}, R^{3d} are hydrogen, and R³ and R^{3a} are independently alkyl, halo, optionally substituted phenyl, —SR¹², or alkyl substituted with one R¹⁶; and all other groups are independently as defined in the Summary of the Invention for a Compound of Formula I or as defined in embodiment (1). In another embodiment, R² is 5,6,7,8-tetrahydroquinazolin-4-yl, 6,7-dihydro-5H-cyclopenta[d]pyrimidin-4-yl, 6,7,8,9-tetrahydro-5H-cyclohepta[d]pyrimidin-4-yl, 5,6-dihydroquinazolin-4-yl, or 7′,8′-dihydro-5′H-spiro[cyclopropane-1,6′-quinazoline]-4′-yl, where R² is substituted with R³, R^{3a}, R^{3b}, R^{3c}, and R^{3d}; where R^{3b}, R^{3c}, R^{3d} are hydrogen, and R³ and R^{3a} are independently alkyl, halo, phenyl, alkyl substituted with one R¹⁶, or —SR¹²; R¹² is alkyl or phenyl; and all other groups are independently as defined in the Summary of the Invention for a Compound of Formula I or as defined in embodiment (1). In another embodiment, R² is 5,6,7,8-tetrahydroquinazolin-4-yl, 6,7-dihydro-5H-cyclopenta[d]pyrimidin-4-yl, 6,7,8,9-tetrahydro-5H-cyclohepta[d]pyrimidin-4-yl, 5,6-dihydroquinazolin-4-yl, or 7′,8′-dihydro-5′H-spiro[cyclopropane-1,6′-quinazoline]-4′-yl, where R² is substituted with R³, R^{3a}, R^{3b}, R^{3c}, and R^{3d}; where R^{3b}, R^{3c}, R^{3d} are hydrogen, R³ is alkyl (in another embodiment alkyl is C₁₋₂-alkyl), and R^{3a} is alkyl (in another embodiment alkyl is C₁₋₂-alkyl), halo, phenyl, alkyl substituted with one R¹⁶, or —SR¹²; R¹² is alkyl or phenyl; and all other groups are independently as defined in the Summary of the Invention for a Compound of Formula I or as defined in embodiment (1). In another embodiment, R² is 5,6,7,8-tetrahydroquinazolin-4-yl, 6,7-dihydro-5H-cyclopenta[d]pyrimidin-4-yl, 6,7,8,9-tetrahydro-5H-cyclohepta[d]pyrimidin-4-yl, 5,6-dihydroquinazolin-4-yl, or 7′,8′-dihydro-5′H-spiro[cyclopropane-1,6′-quinazoline]-4′-yl, where R² is substituted with R³, R^{3a}, R^{3b}, R^{3c}, and R^{3d}; where R^{3b}, R^{3c}, R^{3d} are hydrogen, R³ and R^{3a} are alkyl, (in another embodiment each alkyl is C₁₋₂-alkyl); and all other groups are independently as defined in the Summary of the Invention for a Compound of Formula I or as defined in embodiment (1). In another embodiment, R² is 5,6,7,8-tetrahydroquinazolin-4-yl, 6,7-dihydro-5H-cyclopenta[d]pyrimidin-4-yl, 6,7,8,9-tetrahydro-5H-cyclohepta[d]pyrimidin-4-yl, 5,6-dihydroquinazolin-4-yl, or 7′,8′-dihydro-5′H-spiro[cyclopropane-1,6′-quinazoline]-4′-yl, where R² is substituted with R³, R^{3a}, R^{3b}, R^{3c}, and R^{3d}; where R^{3b}, R^{3c}, R^{3d} are hydrogen, R³ and R^{3a} are halo; and all other groups are independently as defined in the Summary of the Invention for a Compound of Formula I or as defined in embodiment (1). In another embodiment, R² is 5,6,7,8-tetrahydroquinazolin-4-yl, 6,7-dihydro-5H-cyclopenta[d]pyrimidin-4-yl, 6,7,8,9-tetrahydro-5H-cyclohepta[d]pyrimidin-4-yl, 5,6-dihydroquinazolin-4-yl, or 7′,8′-dihydro-5′H-spiro[cyclopropane-1,6′-quinazoline]-4′-yl, where R² is substituted with R³, R^{3a}, R^{3b}, R^{3c}, and R^{3d}; where R^{3b}, R^{3c}, R^{3d} are hydrogen, R³ is alkyl (in another embodiment alkyl is C₁₋₂-alkyl), and R^{3a} is hydrogen, alkyl, or alkyl substituted with R¹⁶; and all other groups are independently as defined in the Summary of the Invention for a Compound of Formula I or as defined in embodiment (1).

### Embodiments (E3d)

In another embodiment, R² is 5,6,7,8-tetrahydroquinazolin-4-yl, 6,7-dihydro-5H-cyclopenta[d]pyrimidin-4-yl, 6,7,8,9-tetrahydro-5H-cyclohepta[d]pyrimidin-4-yl, 5,6-dihydroquinazolin-4-yl, or 7′,8′-dihydro-5′H-spiro[cyclopropane-1,6′-quinazoline]-4′-yl, where R² is substituted with R³, R^{3a}, R^{3b}, R^{3c}, and R^{3d}; where R^{3c}, R^{3d} are hydrogen, and R³, R^{3a}, and R^{3b} are independently alkyl, alkenyl, halo, hydroxyalkyl, cyanoalkyl, alkyl substituted with R¹⁶, heterocycloalkyl, or heterocycloalkylalkyl (optionally substituted with one or two halo); and all other groups are independently as defined in the Summary of the Invention for a Compound of Formula I or as defined in embodiment (1). In another embodiment, R² is 5,6,7,8-tetrahydroquinazolin-4-yl, 6,7-dihydro-5H-cyclopenta[d]pyrimidin-4-yl, 6,7,8,9-tetrahydro-5H-cyclohepta[d]pyrimidin-4-yl, 5,6-dihydroquinazolin-4-yl, or 7′,8′-dihydro-5′H-spiro[cyclopropane-1,6′-quinazoline]-4′-yl, where R² is substituted with R³, R^{3a}, R^{3b}, R^{3c}, and R^{3d}; where R^{3c}, R^{3d} are hydrogen, and R³, R^{3a}, and R^{3b} are independently alkyl, alkenyl, halo, hydroxyalkyl, cyanoalkyl, alkyl substituted with R¹⁶, heterocycloalkyl, or heterocycloalkylalkyl (optionally substituted with one or two halo); R¹⁶ is NR¹¹R^{11a} where R¹¹ is hydrogen or alkyl and R^{11a} is alkyl, haloalkyl, alkoxyalkyl, cycloalkyl, cycloalkylalkyl, or carboxyalkyl; or R¹⁶ is —NR¹⁵S(O)R^{15a} where R¹⁵ and R^{15a} are independently hydrogen or alkyl; or R¹⁶ is —OC(O)R¹⁷ where R¹⁷ is alkyl; R¹⁶ is —OR¹⁸ where R¹⁸ is alkyl or alkoxyalkyl; and all other groups are independently as defined in the Summary of the Invention for a Compound of Formula I or as defined in embodiment (1).

### Embodiments (E3e)

In another embodiment, R² is 5,6,7,8-tetrahydroquinazolin-4-yl, 6,7-dihydro-5H-cyclopenta[d]pyrimidin-4-yl, 6,7,8,9-tetrahydro-5H-cyclohepta[d]pyrimidin-4-yl, 5,6-dihydroquinazolin-4-yl, or 7′,8′-dihydro-5′H-spiro[cyclopropane-1,6′-quinazoline]-4′-yl, where R² is substituted with R³, R^{3a}, R^{3b}, R^{3c}, and R^{3d}; where R^{3c}, are hydrogen, and R³, R^{3a}, and R^{3b} are alkyl (in another embodiment each alkyl is C₁₋₂-alkyl); and all other groups are independently as defined in the Summary of the Invention for a Compound of Formula I or as defined in embodiment (1). In another embodiment, R² is 5,6,7,8-tetrahydroquinazolin-4-yl, 6,7-dihydro-5H-cyclopenta[d]pyrimidin-4-yl, 6,7,8,9-tetrahydro-5H-cyclohepta[d]pyrimidin-4-yl, 5,6-dihydroquinazolin-4-yl, or 7′,8′-dihydro-5′H-spiro[cyclopropane-1,6′-quinazoline]-4′-yl, where R² is substituted with R³, R^{3a}, R^{3b}, R^{3c}, and R^{3d}; where R^{3c}, R^{3d} are hydrogen, R³ and R^{3a} are alkyl (in another embodiment each alkyl is C₁₋₂-alkyl), and R^{3b} is alkyl substituted with R¹⁶; and all other groups are independently as defined in the Summary of the Invention for a Compound of Formula I or as defined in embodiment (1). In another embodiment, R² is 5,6,7,8-tetrahydroquinazolin-4-yl, 6,7-dihydro-5H-cyclopenta[d]pyrimidin-4-yl, 6,7,8,9-tetrahydro-5H-cyclohepta[d]pyrimidin-4-yl, 5,6-dihydroquinazolin-4-yl, or 7′,8′-dihydro-5′H-spiro[cyclopropane-1,6′-quinazoline]-4′-yl, where R² is substituted with R³, R^{3a}, R^{3b}, R^{3c}, and R^{3d}; where R^{3c}, R^{3d} are hydrogen, R³ and R^{3a} are alkyl (in another embodiment each alkyl is C₁₋₂-alkyl), and R^{3b} is heterocycloalkylalkyl; and all other groups are independently as defined in the Summary of the Invention for a Compound of Formula I or as defined in embodiment (1). In another embodiment, R² is 5,6,7,8-tetrahydroquinazolin-4-yl, 6,7-dihydro-5H-cyclopenta[d]pyrimidin-4-yl, 6,7,8,9-tetrahydro-5H-cyclohepta[d]pyrimidin-4-yl, 5,6-dihydroquinazolin-4-yl, or 7′,8′-dihydro-5′H-spiro[cyclopropane-1,6′-quinazoline]-4′-yl, where R² is substituted with R³, R^{3a}, R^{3b}, R^{3c}, and R^{3d}; where R^{3c}, R^{3d} are hydrogen, R³ and R^{3a} are alkyl (in another embodiment each alkyl is C₁₋₂-alkyl), and R^{3b} is heterocycloalkyl; and all other groups are independently as defined in the Summary of the Invention for a Compound of Formula I or as defined in embodiment (1).

### Embodiments (E3f)

In another embodiment, R² is 6,7-dihydro-5H-cyclopenta[d]pyrimidin-4-yl, 6-methyl-6,7-dihydro-5H-cyclopenta[d]pyrimidin-4-yl, 6,6-dimethyl-6,7-dihydro-5H-cyclopenta[d]pyrimidin-4-yl, 6-methyl-2-(methylthio)-6,7-dihydro-5H-cyclopenta[d]pyrimidin-4-yl, 2-(ethylthio)-6,7-dihydro-5H-cyclopenta[d]pyrimidin-4-yl, 2-(phenylmethylthio)-6,7-dihydro-5H-cyclopenta[d]pyrimidin-4-yl, 5-phenyl-6,7-dihydro-5H-cyclopenta[d]pyrimidin-4-yl, 6-phenyl-6,7-dihydro-5H-cyclopenta[d]pyrimidin-4-yl, 5,6,7,8-tetrahydroquinazolin-4-yl, 6-methyl-5,6,7,8-tetrahydroquinazolin-4-yl, 6-ethyl-5,6,7,8-tetrahydroquinazolin-4-yl, 7-methyl-5,6,7,8-tetrahydroquinazolin-4-yl, 7-methyl-7-phenyl-5,6,7,8-tetrahydroquinazolin-4-yl, 6,6-dimethyl-5,6,7,8-tetrahydroquinazolin-4-yl, or 7,7-dimethyl-5,6,7,8-tetrahydroquinazolin-4-yl; and all other groups are independently as defined in the Summary of the Invention for a Compound of Formula I or as defined in embodiment (1).

### Embodiments (E4)

In another embodiment, R² is according to Formula (c)

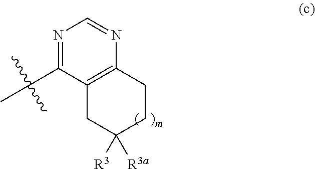

where m is 0 or 1 and R³, R^{3a} and all other groups are independently as defined in the Summary of the Invention for a Compound of Formula I or as defined in embodiment (1). In another embodiment, R² is according to Formula (c) where m is 0 or 1 and R³ and R^{3a}, together with the carbon to which they are attached, form an optionally substituted cycloalkyl or an optionally substituted heterocycloalkyl; and all other groups are independently as defined in the Summary of the Invention for a Compound of Formula I or as defined in embodiment (1). In another embodiment, R² is according to Formula (c) where m is 0 or 1 and R³ and R^{3a} are alkyl (in another embodiment each alkyl is C₁₋₂-alkyl); and all other groups are independently as defined in the Summary of the Invention for a Compound of Formula I or as defined in embodiment (1). In another embodiment, R² is according to Formula (c) where m is 0 or 1 and R³ and R^{3a} are halo; and all other groups are independently as defined in the Summary of the Invention for a Compound of Formula I or as defined in embodiment (1).

### Embodiments (E4a)

In another embodiment, R² is according to formula (c), m is 1, R³ and R^{3a} are as defined in any of the embodiments (E4d); and all other groups are as defined in the Summary of the Invention for a Compound of Formula I or as defined in embodiment (1).

### Embodiments (E4b)

In another embodiment, R² is 6,6-dimethyl-5,6,7,8-tetrahydroquinazolin-4-yl, 6,6-dichloro-5,6,7,8-tetrahydroquinazolin-4-yl, 6,6-difluoro-5,6,7,8-tetrahydroquinazolin-4-yl, 7,7-dimethyl-5,6,7,8-tetrahydroquinazolin-4-yl, 7,7-dichloro-5,6,7,8-tetrahydroquinazolin-4-yl, 7′,8′-dihydro-5′H-spiro[cyclopropane-1,6′-quinazoline]-4′-yl, or 6′,8′-dihydro-5′H-spiro[cyclopropane-1,7′-quinazoline]-4′-yl, where R² is substituted with R^{3b} where R^{3b} is hydrogen, alkyl, or haloalkyl; and all other groups are independently as defined in the Summary of the Invention for a Compound of Formula I or as defined in embodiment (1).

### Embodiments (E4d)

In another embodiment, R² is according to Formula (d)

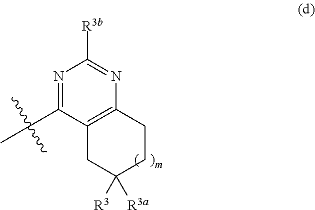

where m is 0 or 1; R³, R^{3a}, R^{3b}, and all other groups are independently as defined in the Summary of the Invention for a Compound of Formula I or as defined in embodiment (1). In another embodiment, R² is according to Formula (d) where m is 0 or 1; R³ and R^{3a} are alkyl (in another embodiment each alkyl is C₁₋₂-alkyl); and all other groups are independently as defined in the Summary of the Invention for a Compound of Formula I or as defined in embodiment (1). In another embodiment, R² is according to Formula (d) where m is 0 or 1; R³ and R^{3a} are halo; and all other groups are independently as defined in the Summary of the Invention for a Compound of Formula I or as defined in embodiment (1). In another embodiment, R² is according to Formula (d) where m is 1; R³ and R^{3a} are alkyl (in another embodiment each alkyl is C₁₋₂-alkyl); and all other groups are independently as defined in the Summary of the Invention for a Compound of Formula I or as defined in embodiment (1). In another embodiment, R² is according to Formula (d) where m is 1; R³ and R^{3a} are halo; and all other groups are independently as defined in the Summary of the Invention for a Compound of Formula I or as defined in embodiment (1). In another embodiment, R² is according to Formula (d) where m is 1; R³ and R^{3a} are alkyl (in another embodiment each alkyl is C₁₋₂-alkyl); R^{3b} is hydrogen, alkyl, alkenyl, hydroxyalkyl, cyanoalkyl, heteorcycloalkyl (optionally substituted with alkoxycarbonyl, benzyloxycarbonyl, or alkyl), heteorcycloalkylalkyl (optionally substituted with one or two halo), or alkyl substituted with one R¹⁶; and all other groups are independently as defined in the Summary of the Invention for a Compound of Formula I or as defined in embodiment (1). In another embodiment, R² is according to Formula (d) where m is 1; R³ and R^{3a} are alkyl (in another embodiment each alkyl is C₁₋₂-alkyl); R^{3b} is hydrogen, alkyl, alkenyl, hydroxyalkyl, cyanoalkyl, heteorcycloalkyl (optionally substituted with alkoxycarbonyl, benzyloxycarbonyl, or alkyl), heteorcycloalkylalkyl (optionally substituted with one or two halo), or alkyl substituted with one R¹⁶; R¹⁶ is —NR¹¹R^{11a}, —NR¹⁵S(O)₂R^{15a}, —OC(O)R¹⁷, or —OR¹⁸; and all other groups are independently as defined in the Summary of the Invention for a Compound of Formula I or as defined in embodiment (1). In another embodiment, R² is according to Formula (d) where m is 1; R³ and R^{3a} are alkyl (in another embodiment each alkyl is C₁₋₂-alkyl); R^{3b} is hydrogen, alkyl (in another embodiment alkyl is C₁₋₂-alkyl), cyanoalkyl, or alkyl substituted with one R¹⁶; and all other groups are independently as defined in the Summary of the Invention for a Compound of Formula I or as defined in embodiment (1).

In another embodiment, the Compound is according to Formula I(a), R² is according to embodiments (E4d) and R¹ is according to embodiments (Z)-(Z5).

### Embodiments (E5a)

In another embodiment, R² is according to Formula (e)

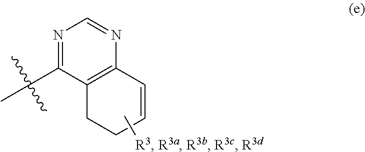

where R³, R^{3a}, R^{3b}, R^{3c}, and R^{3d} are positioned on any substitutable carbon of ring (e); and all other groups are independently as defined in the Summary of the Invention for a Compound of Formula I or as defined in embodiment (1). In another embodiment, R² is according to Formula (e) where one of R³, R^{3a}, R^{3b}, R^{3c}, and R^{3d} is hydrogen, alkyl (in another embodiment each alkyl is C₁₋₂-alkyl), or alkyl substituted with one R¹⁶ and the other of R³, R^{3a}, R^{3b}, R^{3c}, and R^{3d} and all other groups are independently as defined in the Summary of the Invention for a Compound of Formula or as defined in embodiment (1). In another embodiment, R² is according to Formula (e) where one of R³, R^{3a}, R^{3b}, R^{3c}, and R^{3d} is hydrogen, alkyl (in another embodiment alkyl is C₁₋₂-alkyl), or alkyl substituted with one R¹⁶ and the other of R³, R^{3a}, R^{3b}, R^{3c}, and R^{3d} are independently hydrogen or alkyl (in another embodiment each alkyl is C₁₋₂-alkyl); and all other groups are as defined in the Summary of the Invention for a Compound of Formula or as defined in embodiment (1). In another embodiment, R² is according to Formula (e) where one of R³, R^{3a}, R^{3b}, R^{3c}, and R^{3d} is hydrogen, alkyl (in another embodiment each alkyl is C₁₋₂-alkyl), or alkyl substituted with one R¹⁶ and the other of R³, R^{3a}, R^{3b}, R^{3c}, and R^{3d} are alkyl, (in another embodiment each alkyl is C₁₋₂-alkyl); and all other groups are as defined in the Summary of the Invention for a Compound of Formula or as defined in embodiment (1). In another embodiment, R² is according to Formula (e) where one of R³, R^{3a}, R^{3b}, R^{3c}, and R^{3d} is hydrogen, alkyl (in another embodiment alkyl is C₁₋₂-alkyl), or alkyl substituted with one R¹⁶, a second of R³, R^{3a}, R^{3b}, R^{3c}, and R^{3d} is hydrogen, and the other of R³, R^{3a}, R^{3c}, and R^{3d} are alkyl (in another embodiment each alkyl is C₁₋₂-alkyl); and all other groups are as defined in the Summary of the Invention for a Compound of Formula or as defined in embodiment (1).

In another embodiment, the Compound is according to Formula I(a), R² is according to embodiments (E5a) and R¹ is according to embodiments (Z)-(Z5).

### Embodiments (E5b)

In another embodiment, R² is according to Formula (f)

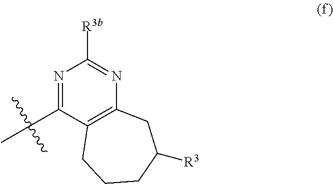

where R^{3b} is hydrogen, alkyl (in another embodiment alkyl is C₁₋₃-alkyl), cyanoalkyl, or alkyl substituted with one R¹⁶; and R³ is hydrogen, alkyl (in another embodiment alkyl is C₁₋₃-alkyl), or alkenyl; and all other groups are as defined in the Summary of the Invention for a Compound of Formula or as defined in embodiment (1).

In another embodiment, the Compound is according to Formula I(a), R² is according to embodiments (E5b) and R¹ is according to embodiments (Z)-(Z5).

### Embodiments (E5c)

In another embodiment, R² is according to Formula (g)

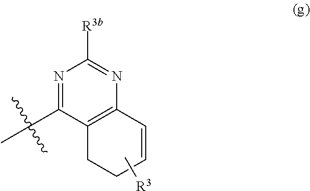

where R^{3b} is hydrogen, alkyl (in another embodiment alkyl is C₁₋₃-alkyl), cyanoalkyl, or alkyl substituted with one R¹⁶; and R³ is alkyl (in another embodiment alkyl is C₁₋₃-alkyl), hydroxyalkyl, alkoxyalkyl, or haloalkyl, and is located at the 6- or 7-position of the ring; and all other groups are as defined in the Summary of the Invention for a Compound of Formula or as defined in embodiment (1).

In another embodiment, the Compound is according to Formula I(a), R² is according to embodiments (E5c) and R¹ is according to embodiments (Z)-(Z5).

### Embodiments (E5d)

In another embodiment, R² is according to Formula (h)

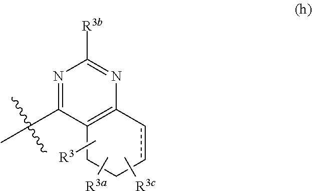

where R³, R^{3a}, R^{3b}, and R^{3c} and all other groups are as defined in the Summary of the Invention for a Compound of Formula or as defined in embodiment (1). In another embodiment, R² is according to Formula (h) where R^{3b} is hydrogen, alkyl, cyanoalkyl, or alkyl substituted with one R¹⁶; and all other groups are as defined in the Summary of the Invention for a Compound of Formula or as defined in embodiment (1). In another embodiment, R² is according to Formula (h) where R^{3b} is hydrogen, cyanoalkyl, alkyl (in another embodiment alkyl is C₁₋₃-alkyl), or alkyl substituted with one R¹⁶; R³, R^{3a}, and R^{3c} are independently hydrogen, alkyl (in another embodiment alkyl is C₁₋₃-alkyl), alkenyl, halo, haloalkyl, hydroxyalkyl, —SR¹², optionally substituted phenyl, —OR^{11a}, alkyl substituted with one R¹⁶, optionally substituted heterocycloalkyl, optionally substituted heterocycloalkylalkyl, or optionally substituted heteroaryl; and all other groups are as defined in the Summary of the Invention for a Compound of Formula or as defined in embodiment (1).

In another embodiment, the Compound is according to Formula I(a), R² is according to embodiments (E5d) and R¹ is according to embodiments (Z)-(Z5).

### Embodiments (E6)

In another embodiment, R² is quinolin-2-yl, quinolin-3-yl, quinolin-4-yl, quinolin-5-yl, quinolin-6-yl, quinolin-7-yl, quinolin-8-yl, isoquinolin-1-yl, isoquinolin-3-yl, isoquinolin-4-yl, isoquinolin-5-yl, isoquinolin-6-yl, isoquinolin-7-yl, or isoquinolin-8-yl, where R² is substituted with R³, R^{3a}, R^{3b}, and R^{3c}; where R³, R^{3a}, R^{3b}, and R^{3c} and all other groups are independently as defined in the Summary of the Invention for a Compound of Formula I or as defined in embodiment (1). In another embodiment, R² is quinolin-4-yl or isoquinolin-1-yl, where R² is substituted with R³, R^{3a}, R^{3b}, R^{3c}, and R^{3d}; where R³, R^{3a}, R^{3b}, R^{3c}, and R^{3d} and all other groups are independently as defined in the Summary of the Invention for a Compound of Formula I or as defined in embodiment (1).

### Embodiments (E6a)

In another embodiment, R² is quinolin-4-yl, quinolin-3-yl, quinolin-4-yl, quinolin-5-yl, quinolin-6-yl, quinolin-7-yl, quinolin-8-yl, isoquinolin-1-yl, isoquinolin-3-yl, isoquinolin-4-yl, isoquinolin-5-yl, isoquinolin-6-yl, isoquinolin-7-yl, or isoquinolin-8-yl, where R² is substituted with R³, R^{3a}, R^{3b}, R^{3c}, and R^{3d}; R^{3b}, R^{3c}, and R^{3d} are hydrogen; R³ and R^{3a} are independently hydrogen, cyano, alkyl, halo, haloalkyl, —OR^{11a}, phenyl, phenylalkyl optionally substituted with one or two R¹⁹, or alkyl substituted with one or two R¹⁶; and all other groups are independently as defined in the Summary of the Invention for a Compound of Formula I or as defined in embodiment (1). In another embodiment, R² is quinolin-4-yl or isoquinolin-1-yl, where R² is substituted with R³, R^{3a}, R^{3b}, R^{3c}, and R^{3d}; R^{3b}, R^{3c}, and R^{3d} are hydrogen; R³ and R^{3a} are independently R³ and R^{3a} are independently hydrogen, cyano, alkyl (in another embodiment alkyl is C₁₋₃-alkyl), halo, haloalkyl, —OR^{11a}, phenyl, phenylalkyl optionally substituted with one or two R¹⁹, or alkyl substituted with one or two R¹⁶; and all other groups are independently as defined in the Summary of the Invention for a Compound of Formula I or as defined in embodiment (1).

### Embodiments (E6b)

In another embodiment, R² is 6,7-dimethoxy-quinolin-4-yl, 7-cyano-quinolin-4-yl, 5-fluoro-quinolin-4-yl, 6-fluoro-quinolin-4-yl, 7-fluoro-quinolin-4-yl, 8-fluoro-quinolin-4-yl, 2-phenyl-quinolin-4-yl, 2-methyl-quinolin-4-yl, 2-methyl-7-methoxy-quinolin-4-yl, 2-trifluoromethyl-quinolin-4-yl, or isoquinolin-1-yl; and all other groups are independently as defined in the Summary of the Invention for a Compound of Formula I or as defined in embodiment (1).

### Embodiments (E7)

In another embodiment, R² is 5H-pyrrolo[3,2-d]pyrimidin-4-yl, thieno[2,3-d]pyrimidin-4-yl, 7H-pyrrolo[2,3-d]pyrimidin-4-yl, 1H-pyrrolo[2,3-b]pyridin-4-yl, 1H-pyrrolo[3,2-c]pyridin-4-yl, thieno[2,3-b]pyridin-4-yl, or thieno[3,2-c]pyridin-4-yl, where R² is substituted with R³, R^{3a}, R^{3b}, R^{3c}, and R^{3d}; R³, R^{3a}, R^{3b}, R^{3c}, and R^{3d} and all other groups are independently as defined in the Summary of the Invention for a Compound of Formula I or as defined in embodiment (1). In another embodiment, R² is thieno[2,3-d]pyrimidin-4-yl or 7H-pyrrolo[2,3-d]pyrimidin-4-yl, where R² is substituted with R³, R^{3a}, R^{3b}, R^{3c}, and R^{3d}; R³, R^{3a}, R^{3b}, R^{3c}, and R^{3d} and all other groups are independently as defined in the Summary of the Invention for a Compound of Formula I or as defined in embodiment (1). In another embodiment, R² is thieno[2,3-d]pyrimidin-4-yl or 7H-pyrrolo[2,3-d]pyrimidin-4-yl, where R² is substituted with R³, R^{3a}, R^{3b}, R^{3c}, and R^{3d}; R^{3a}, R^{3b}, R^{3c}, and R^{3d} are hydrogen; R³ is hydrogen or alkyl (in another embodiment alkyl is C₁₋₃-alkyl); and all other groups are independently as defined in the Summary of the Invention for a Compound of Formula I or as defined in embodiment (1). In another embodiment, R² is thieno[2,3-d]pyrimidin-4-yl, 5-methyl-thieno[2,3-d]pyrimidin-4-yl, or 7H-pyrrolo[2,3-d]pyrimidin-4-yl; and all other groups are independently as defined in the Summary of the Invention for a Compound of Formula I or as defined in embodiment (1).

### Embodiments (E8)

In another embodiment, R² is 5,7-dihydrothieno[3,4-d]pyrimidin-4-yl, 5,6,7,8-tetrahydropyrido[3,4-d]pyrimidin-4-yl, 5,6,7,8-tetrahydropyrido[4,3-a]pyrimidin-4-yl, 5,6,7,8-tetrahydropyrido[2,3-d]pyrimidin-4-yl, 5,6,7,8-tetrahydropyrido[3,2-d]pyrimidin-4-yl, 6,7-dihydro-5H-pyrrolo[3,4-d]pyrimidin-4-yl, 6,7-dihydro-5H-pyrrolo[3,2-d]pyrimidin-4-yl, or 6,7-dihydro-5H-pyrrolo[2,3-d]pyrimidin-4-yl, where R² is substituted with R³, R^{3a}, R^{3b}, R^{3c}, and R^{3d}; where R³, R^{3a}, R^{3b}, R^{3c}, and R^{3d} and all other groups are independently as defined in the Summary of the Invention for a Compound of Formula I or as defined in embodiment (1).

### Embodiments (E8a)

In another embodiment, R² is 5,7-dihydrothieno[3,4-d]pyrimidin-4-yl, 5,6,7,8-tetrahydropyrido[3,4-d]pyrimidin-4-yl, 5,6,7,8-tetrahydropyrido[4,3-d]pyrimidin-4-yl, or 6,7-dihydro-5H-pyrrolo[3,4-d]pyrimidin-4-yl, where R² is substituted with R³, R^{3a}, R^{3b}, R^{3c}, and R^{3d}; R³, R^{3a}, R^{3b}, R^{3c}, and R^{3d} and all other groups are independently as defined in the Summary of the Invention for a Compound of Formula I or as defined in embodiment (1).

### Embodiments (E8b)

In another embodiment, R² is 5,7-dihydrothieno[3,4-d]pyrimidin-4-yl, 5,6,7,8-tetrahydropyrido[3,4-d]pyrimidin-4-yl, 5,6,7,8-tetrahydropyrido[4,3-d]pyrimidin-4-yl, or 6,7-dihydro-5H-pyrrolo[3,4-d]pyrimidin-4-yl, where R² is substituted with R³, R^{3a}, R^{3b}, R^{3c}, and R^{3d}; R³, R^{3a}, R^{3b}, R^{3c}, and R^{3d} and all other groups are independently as defined in the Summary of the Invention for a Compound of Formula I or as defined in embodiment (1). In another embodiment, R² is 5,7-dihydrothieno[3,4-d]pyrimidin-4-yl, 5,6,7,8-tetrahydropyrido[3,4-d]pyrimidin-4-yl, 5,6,7,8-tetrahydropyrido[4,3-d]pyrimidin-4-yl, or 6,7-dihydro-5H-pyrrolo[3,4-d]pyrimidin-4-yl, where R² is substituted with R³, R^{3a}, R^{3b}, R^{3c}, and R^{3d}; R^{3a}, R^{3b}, R^{3c}, and R^{3d} are hydrogen; R³ is hydrogen, alkyl (in another embodiment alkyl is C₁₋₃-alkyl), haloalkyl, optionally substituted phenyl, optionally substituted phenylalkyl, optionally substituted cycloalkyl, or optionally substituted cycloalkylalkyl; and all other groups are independently as defined in the Summary of the Invention for a Compound of Formula I or as defined in embodiment (1).

### Embodiments (E8c)

In another embodiment, R² is 5,7-dihydrothieno[3,4-d]pyrimidin-4-yl, 5,6,7,8-tetrahydropyrido[3,4-d]pyrimidin-4-yl, 7-ethyl-5,6,7,8-tetrahydropyrido[3,4-d]pyrimidin-4-yl, 7-benzyl-5,6,7,8-tetrahydropyrido[3,4-d]pyrimidin-4-yl, 5,6,7,8-tetrahydropyrido[4,3-d]pyrimidin-4-yl, 6-cyclopropyl-5,6,7,8-tetrahydropyrido[4,3-c]pyrimidin-4-yl, 6,7-dihydro-5H-pyrrolo[3,4-d]pyrimidin-4-yl, 6-p-tolyl-6,7-dihydro-5H-pyrrolo[3,4-d]pyrimidin-4-yl, or 6-cyclopropyl-6,7-dihydro-5H-pyrrolo[3,4-d]pyrimidin-4-yl; and all other groups are independently as defined in the Summary of the Invention for a Compound of Formula I or as defined in embodiment (1).

### Embodiments (E9)

In another embodiment, R² is 7H-pyrrolo[2,3-d]pyrimidin-4-yl substituted with R³, R^{3a}, R^{3b}, R^{3c}, and R^{3d}; R^{3a}, R^{3b}, R^{3c}, and R^{3d} are hydrogen; R³ and all other groups are independently as defined in the Summary of the Invention for a Compound of Formula I or as defined in embodiment (1). In another embodiment, R² is 7H-pyrrolo[2,3-d]pyrimidin-4-yl substituted with R³, R^{3a}, R^{3b}, R^{3c}, and R^{3d}; R³, R^{3a}, R^{3b}, R^{3c}, and R^{3d} are hydrogen; and all other groups are independently as defined in the Summary of the Invention for a Compound of Formula I or as defined in embodiment (1).

### Embodiments (E10)

In another embodiment, R² is 1H-pyrazolo[3,4-d]pyrimidin-4-yl substituted with R³, R^{3a}, R^{3b}, R^{3c}, and R^{3d}; R^{3a}, R^{3b}, R^{3c}, and R^{3d} are hydrogen; R³ and all other groups are independently as defined in the Summary of the Invention for a Compound of Formula I or as defined in embodiment (1). In another embodiment, R² is 1H-pyrazolo[3,4-d]pyrimidin-4-yl substituted with R³, R^{3a}, R^{3b}, R^{3c}, and R^{3d}; R³, R^{3a}, R^{3b}, R^{3c}, and R^{3d} are hydrogen; and all other groups are independently as defined in the Summary of the Invention for a Compound of Formula I or as defined in embodiment (1).

### Embodiments (E11)

In another embodiment, R² is 6,7,8,9-tetrahydropyrimido[4,5-b]indolizin-4-yl substituted with R³, R^{3a}, R^{3b}, R^{3c}, and R^{3d}; where R³, R^{3a}, R^{3b}, R^{3c}, and R^{3d} and all other groups are independently as defined in the Summary of the Invention for a Compound of Formula I or as defined in embodiment (1). In another embodiment, R² is 6,7,8,9-tetrahydropyrimido[4,5-b]indolizin-4-yl substituted with R³, R^{3a}, R^{3b}, R^{3c}, and R^{3d}; R^{3a}, R^{3b}, R^{3c}, and R^{3d} are hydrogen; R³ is hydrogen or cyano; and all other groups are independently as defined in the Summary of the Invention for a Compound of Formula I or as defined in embodiment (1). In another embodiment, R² is 6,7,8,9-tetrahydropyrimido[4,5-b]indolizin-4-yl or 10-cyano-6,7,8,9-tetrahydropyrimido[4,5-b]indolizin-4-yl; and all other groups are independently as defined in the Summary of the Invention for a Compound of Formula I or as defined in embodiment (1).

In another embodiment, the Compound is according to any of embodiments (B) and (H1) and R² is according to any one of embodiments (D)-(D2), (D3)-(D3k), (D4)-(D4b), (D5), (D6-D6d), (D7)-(D7d), (E)-(E2), (E2a)-(E2e), (E3)-(E3f), (E4)-(E4d), (E5a)-(E5d), (E6)-(E6b), (E7), (E8)-(E8c), and (E9)-(E11). In another embodiment, the Compound is according to any of embodiments (B) and (H1) and R² is according to any one of embodiments (D2), (D3a)-(D3c), (D3g), (D3i), (E2), (E2b), (E3c), (E4a), (E4d), and (E5a)-(E5d).

In another embodiment, the Compound is according to any of embodiments (B1)-(B2) and R² is according to any one of embodiments (D)-(D2), (D3)-(D3k), (D4)-(D4b), (D5), (D6-D6d), (D7)-(D7d), (E)-(E2), (E2a)-(E2e), (E3)-(E3f), (E4)-(E4d), (E5a)-(E5d), (E6)-(E6b), (E7), (E8)-(E8c), and (E9)-(E11). In another embodiment, the Compound is according to any of embodiments (B1) and R² is according to any one of embodiments (D2), (D3a)-(D3c), (D3g), (D3i), (E2), (E2b), (E3c), (E4a), (E4d), and (E5a)-(E5d).

In another embodiment, the Compound is according to any of embodiments (B3), (B4), (B4a), and (B4b) and R² is according to any one of embodiments (D)-(D2), (D3)-(D3k), (D4)-(D4b), (D5), (D6-D6d), (D7)-(D7d), (E)-(E2), (E2a)-(E2e), (E3)-(E3f), (E4)-(E4d), (E5a)-(E5d), (E6)-(E6b), (E7), (E8)-(E8c), and (E9)-(E11). In another embodiment, the Compound is according to any of embodiments (B4a) and R² is according to any one of embodiments (D2), (D3a)-(D3c), (D3g), (D3i), (E2), (E2b), (E3c), (E4a), (E4d), and (E5a)-(E5d).

In another embodiment, the Compound is according to any of embodiments (B5), (B6), (B7), and (B8) and R² is according to any one of embodiments (D)-(D2), (D3)-(D3k), (D4)-(D4b), (D5), (D6-D6d), (D7)-(D7d), (E)-(E2), (E2a)-(E2e), (E3)-(E3f), (E4)-(E4d), (E5a)-(E5d), (E6)-(E6b), (E7), (E8)-(E8c), and (E9)-(E11). In another embodiment, the Compound is according to any of embodiments (B7) and R² is according to any one of embodiments (D2), (D3a)-(D3c), (D3g), (D3i), (E2), (E2b), (E3c), (E4a), (E4d), and (E5a)-(E5d).

In another embodiment, the Compound is according to any of embodiments (B9)-(B13) and R² is according to any one of embodiments (D)-(D2), (D3)-(D3k), (D4)-(D4b), (D5), (D6-D6d), (D7)-(D7d), (E)-(E2), (E2a)-(E2e), (E3)-(E30, (E4)-(E4d), (E5a)-(E5d), (E6)-(E6b), (E7), (E8)-(E8c), and (E9)-(E11). In another embodiment, the Compound is according to any of embodiments (B9)-(B13) and R² is according to any one of embodiments (D2), (D3a)-(D3c), (D3g), (D3i), (E2), (E2b), (E3c), (E4a), (E4d), and (E5a)-(E5d).

In another embodiment, the Compound is according to any of embodiments (B16), (B16a)-(B16c), (B17), and (B18) and R² is according to any one of embodiments (D)-(D2), (D3)-(D3k), (D4)-(D4b), (D5), (D6-D6d), (D7)-(D7d), (E)-(E2), (E2a)-(E2e), (E3)-(E3f), (E4)-(E4d), (E5a)-(E5d), (E6)-(E6b), (E7), (E8)-(E8c), and (E9)-(E11). In another embodiment, the Compound is according to any of embodiments (B16a)-(B16c) and R² is according to any one of embodiments (D)-(D2), (D3)-(D3k), (D4)-(D4b), (D5), (D6-D6d), (D7)-(D7d), (E)-(E2), (E2a)-(E2e), (E3)-(E3f), (E4)-(E4d), (E5a)-(E5d), (E6)-(E6b), (E7), (E8)-(E8c), and (E9)-(E 11). In another embodiment, the Compound is according to any of embodiments (B16a)-(B16c) and R² is according to any one of embodiments (D2), (D3a)-(D3c), (D3g), (D3i), (E2), (E2b), (E3c), (E4a), (E4d), and (E5a)-(E5d).

In another embodiment, the Compound is according to any of embodiments (B19)-(B29) and R² is according to any one of embodiments (D)-(D2), (D3)-(D3k), (D4)-(D4b), (D5), (D6-D6d), (D7)-(D7d), (E)-(E2), (E2a)-(E2e), (E3)-(E30, (E4)-(E4d), (E5a)-(E5d), (E6)-(E6b), (E7), (E8)-(E8c), and (E9)-(E11). In another embodiment, the Compound is according to any of embodiments (B19)-(B29) and R² is according to any one of embodiments (D2), (D3a)-(D3c), (D3g), (D3i), (E2), (E2b), (E3c), (E4a), (E4d), and (E5a)-(E5d).

In another embodiment, the Compound is according to any of embodiments (C)-(C3) and R² is according to any one of embodiments (D)-(D2), (D3)-(D3k), (D4)-(D4b), (D5), (D6-D6d), (D7)-(D7d), (E)-(E2), (E2a)-(E2e), (E3)-(E3f), (E4)-(E4d), (E5a)-(E5d), (E6)-(E6b), (E7), (E8)-(E8c), and (E9)-(E11). In another embodiment, the Compound is according to any of embodiments (C2) and R² is according to any one of embodiments (D)-(D2), (D3)-(D3k), (D4)-(D4b), (D5), (D6-D6d), (D7)-(D7d), (E)-(E2), (E2a)-(E2e), (E3)-(E3f), (E4)-(E4d), (E5a)-(E5d), (E6)-(E6b), (E7), (E8)-(E8c), and (E9)-(E11). In another embodiment, the Compound is according to any of embodiments (C2) and R² is according to any one of embodiments (D2), (D3a)-(D3c), (D3g), (D3i), (E2), (E2b), (E3c), (E4a), (E4d), and (E5a)-(E5d).

### Embodiments Z

In another embodiment, the Compound is that where R¹ is benzimidazol-6-yl optionally substituted with one or two R⁷; and R⁷ is as defined in the Summary of the Invention for a Compound of Formula I or as defined in embodiment (1). In another embodiment, the Compound is that where R¹ is benzimidazol-6-yl optionally NR⁸R^{8a}, substituted with one or two R⁷; each R⁷, when present, is alkyl, haloalkyl, —NR⁸R^{8a}, —NR⁸C(O)OR⁹, or cycloalkyl; and R⁸, R^{8a}, and R⁹ are independently as defined in the Summary of the Invention for a Compound of Formula I or as defined in embodiment (1). In another embodiment, the Compound is that where R¹ is benzimidazol-6-yl optionally substituted with one or two R⁷; each R⁷, when present, is independently alkyl (in another embodiment alkyl is C₁₋₃-alkyl), haloalkyl, —NR⁸R^{8a}, —NR⁸C(O)OR⁹, or cycloalkyl; R⁸ is hydrogen; R^{8a} is hydrogen, alkyl (in another embodiment alkyl is C₁₋₃-alkyl), or haloalkyl; R⁹ is hydrogen or alkyl (in another embodiment alkyl is C₁₋₃-alkyl).

### Embodiments Z1

In another embodiment, the Compound is that where R¹ is thiazolo[5,4-b]pyridin-6-yl or thiazolo[4,5-b]pyridin-6-yl optionally substituted with one or two R⁷; and R⁷ is as defined in the Summary of the Invention for a Compound of Formula I or as defined in embodiment (1). In another embodiment, the Compound is that where R¹ is thiazolo[5,4-b]pyridin-6-yl or thiazolo[4,5-b]pyridin-6-yl optionally substituted with one or two R⁷; each R⁷, when present, is independently alkyl, haloalkyl, —NR⁸R^{8a}, —NR⁸C(O)OR⁹, or cycloalkyl; and R⁸, R^{8a}, and R⁹ are independently as defined in the Summary of the Invention for a Compound of Formula I or as defined in embodiment (1). In another embodiment, the Compound is that where R¹ is thiazolo[5,4-b]pyridin-6-yl or thiazolo[4,5-b]pyridin-6-yl optionally substituted with one or two R⁷; each R⁷, when present, is independently alkyl (in another embodiment alkyl is C₁₋₃-alkyl), haloalkyl, —NR⁸R^{8a}, —NR⁸C(O)OR⁹, or cycloalkyl; R⁸ is hydrogen; R^{8a} is hydrogen, alkyl (in another embodiment alkyl is C₁₋₃-alkyl), or haloalkyl; R⁹ is hydrogen or alkyl (in another embodiment alkyl is C₁₋₃-alkyl).

### Embodiments Z2

In another embodiment, the Compound is that where R¹ is 1H-imidazo[4,5-b]pyridin-5-yl, 1H-imidazo[4,5-b]pyridin-6-yl, 3H-imidazo[4,5-b]pyridin-5-yl, or 3H-imidazo[4,5-b]pyridin-6-yl where R¹ is optionally substituted with R⁷; and R⁷ is as defined in the Summary of the Invention for a Compound of Formula I or as defined in embodiment (1). In another embodiment, the Compound is that where R¹ is 1H-imidazo[4,5-b]pyridin-5-yl, 1H-imidazo[4,5-b]pyridin-6-yl, 3H-imidazo[4,5-b]pyridin-5-yl, or 3H-imidazo[4,5-b]pyridin-6-yl where R¹ is optionally substituted with one or two R⁷; each R⁷, when present, is independently alkyl (in another embodiment alkyl is C₁₋₃-alkyl), haloalkyl, —NR⁸R^{8a}, —NR⁸C(O)OR⁹, or cycloalkyl; and R⁸, R^{8a}, and R⁹ are independently as defined in the Summary of the Invention for a Compound of Formula I or as defined in embodiment (1). In another embodiment, the Compound is that where R¹ is 1H-imidazo[4,5-b]pyridin-5-yl, 1H-imidazo[4,5-b]pyridin-6-yl, 3H-imidazo[4,5-b]pyridin-5-yl, or 3H-imidazo[4,5-b]pyridin-6-yl where R¹ is optionally substituted with R⁷; each R⁷, when present, is independently alkyl (in another embodiment alkyl is C₁₋₃-alkyl), haloalkyl, —NR⁸R^{8a}, —NR⁸C(O)OR⁹, or cycloalkyl; R⁸ is hydrogen; R^{8a} is hydrogen, alkyl (in another embodiment alkyl is C₁₋₃-alkyl), or haloalkyl; R⁹ is hydrogen or alkyl (in another embodiment alkyl is C₁₋₃-alkyl).

### Embodiments Z3

In another embodiment, the Compound is that where W is 1H-imidazo[4,5-c]pyridin-6-yl or 3H-imidazo[4,5-c]pyridin-6-yl optionally substituted with one or two R⁷; and R⁷ is as defined in the Summary of the Invention for a Compound of Formula I or as defined in embodiment (1). In another embodiment, the Compound is that where R¹ is 1H-imidazo[4,5-c]pyridin-6-yl or 3H-imidazo[4,5-c]pyridin-6-yl optionally substituted with one or two R⁷; each R⁷, when present, is independently alkyl (in another embodiment alkyl is C₁₋₃-alkyl), haloalkyl, —NR⁸R^{8a}, —NR⁸C(O)OR⁹, or cycloalkyl; and R⁸, R^{8a}, and R⁹ are independently as defined in the Summary of the Invention for a Compound of Formula I or as defined in embodiment (1). In another embodiment, the Compound is that where R¹ is 1H-imidazo[4,5-c]pyridin-6-yl or 3H-imidazo[4,5-c]pyridin-6-yl optionally substituted with one or two R⁷; each R⁷, when present, is independently alkyl (in another embodiment alkyl is C₁₋₃-alkyl), haloalkyl, —NR⁸R^{8a}, —NR⁸C(O)OR⁹, or cycloalkyl; R⁸ is hydrogen; R^{8a} is hydrogen, alkyl (in another embodiment alkyl is C₁₋₃-alkyl), or haloalkyl; R⁹ is hydrogen or alkyl (in another embodiment alkyl is C₁₋₃-alkyl).

### Embodiments Z4

In another embodiment, the Compound is that where R¹ is benzo[d]thiazol-5-yl or benzo[d]thiazol-6-yl optionally substituted with one or two R⁷; and R⁷ is as defined in the Summary of the Invention for a Compound of Formula I or as defined in embodiment (1). In another embodiment, the Compound is that where R¹ is benzo[d]thiazol-5-yl or benzo[d]thiazol-6-yl optionally substituted with one or two R⁷; each R⁷, when present, is independently alkyl (in another embodiment alkyl is C₁₋₃-alkyl), haloalkyl, —NR⁸R^{8a}, —NR⁸C(O)OR⁹, or cycloalkyl; and R⁸, R^{8a}, and R⁹ are independently as defined in the Summary of the Invention for a Compound of Formula I or as defined in embodiment (1). In another embodiment, the Compound is that where R¹ is benzo[d]thiazol-5-yl or benzo[d]thiazol-6-yl optionally substituted with one or two R⁷; each R⁷, when present, is independently alkyl (in another embodiment alkyl is C₁₋₃-alkyl), haloalkyl, —NR⁸R^{8a}, —NR⁸C(O)OR⁹, or cycloalkyl; R⁸ is hydrogen; R^{8a} is hydrogen, alkyl (in another embodiment alkyl is C₁₋₃-alkyl), or haloalkyl; R⁹ is hydrogen or alkyl (in another embodiment alkyl is C₁₋₃-alkyl).

### Embodiments Z5

In another embodiment, the Compound is that where R¹ is pyridin-3-yl optionally substituted with one or two R⁷; and R⁷ is as defined in the Summary of the Invention for a Compound of Formula I or as defined in embodiment (1). In another embodiment, the Compound is that where R¹ is pyridin-3-yl optionally substituted with one or two R⁷; each R⁷, when present, is independently hydrogen, halo, cyano, hydroxy, alkoxy, alkyl, —NR⁸R^{8a}, —NR⁸S(O)₂R^{8a}, —S(O)R¹³, —S(O)₂R^{13a}, or —S(O)₂NR⁸R⁹; and all other groups are independently as defined in the Summary of the Invention for a Compound of Formula I or as defined in embodiment (1). In another embodiment, the Compound is that where R¹ is pyridin-3-yl optionally substituted with two R⁷; one R⁷ is hydrogen, halo, cyano, alkoxy, alkyl (in another embodiment alkyl is C₁₋₃-alkyl), or —NR⁸R^{8a} and the other R⁷ is —NR⁸S(O)₂R^{8a}; or one R⁷ is hydroxy or —NR⁸R^{8a} and the other R⁷ is —S(O)R¹³, —S(O)₂R^{13a}, —S(O)₂NR⁸R⁹; and all other groups are independently as defined in the Summary of the Invention for a Compound of Formula I or as defined in embodiment (1). In another embodiment, the Compound is that where R¹ is pyridin-3-yl optionally substituted with two R⁷; one R⁷ is hydrogen, halo, cyano, alkoxy, alkyl (in another embodiment alkyl is C₁₋₃-alkyl), or —NR⁸R^{8a} and the other R⁷ is —NR⁸S(O)₂R^{8a}; or one R⁷ is hydroxy or —NR⁸R^{8a} and the other R⁷ is —S(O)R¹³, —S(O)₂R^{13a}, —S(O)₂NR⁸R⁹; R¹³ is hydroxyalkyl; R^{13a} is alkyl or heterocycloalkyl optionally substituted with one group which is amino, alkyl, hydroxyalkyl, or hydroxy; each R⁸ and R^{8a} are independently hydrogen or alkyl; R⁹ is hydrogen, haloalkyl, alkoxyalkyl, hydroxyalkyl, aminoalkyl, alkylaminoalkyl, dialkylaminoalkyl, cycloalkyl, heterocycloalkyl, heterocycloalkylalkyl, alkyl substituted with one aminocarbonyl, or hydroxyalkyl which is substituted with one amino or 3 halo; and all other groups are independently as defined in the Summary of the Invention for a Compound of Formula I or as defined in embodiment (1).

### Embodiments (X)

In another embodiment, the Compound is that where R⁶ is —S(O)₂R⁸, —C(O)NR⁸R^{8a} or heteroaryl optionally substituted with 1, 2, or 3 R¹⁴; and R⁸, R^{8a}, and R¹⁴ are independently as defined in the Summary of the Invention for a Compound of Formula I or as defined in embodiment (1). In another embodiment, the Compound is that where R⁶ is located in the para position of the phenyl ring to which it is attached; R⁶ is —C(O)NR⁸R^{8a} or heteroaryl optionally substituted with 1, 2, or 3 R¹⁴; and R⁸, R^{8a}, and R¹⁴ are independently as defined in the Summary of the Invention for a Compound of Formula I or as defined in embodiment (1). In another embodiment, the Compound is that where R⁶ is located in the para position of the phenyl ring to which it is attached; R⁶ is —C(O)NR⁸R^{8a} or heteroaryl optionally substituted with 1, 2, or 3 R¹⁴; R⁸ is hydrogen; R^{8a} is hydrogen, alkyl (in another embodiment alkyl is C₁₋₃-alkyl), haloalkyl, or optionally substituted heterocycloalkyl; R¹⁴ is alkyl (in another embodiment alkyl is C₁₋₃-alkyl) or alkoxycarbonyl. In another embodiment, the Compound is that where R⁶ is located in the para position of the phenyl ring to which it is attached; R⁶ is —C(O)NR⁸R^{8a}, imidazolyl, or pyrazolyl where the imidazolyl and pyrazolyl are optionally substituted with 1, 2, or 3 R¹⁴; R⁸ is hydrogen; R^{8a} is hydrogen, alkyl (in another embodiment alkyl is C₁₋₃-alkyl), haloalkyl, or optionally substituted pyrrolidinyl; R¹⁴ is alkyl (in another embodiment alkyl is C₁₋₃-alkyl) or alkoxycarbonyl. In another embodiment, the Compound is that where R⁶ is located in the meta position of the phenyl ring to which it is attached; R⁶ is —S(O)₂R⁸; and R⁸ is as defined in the Summary of the Invention for a Compound of Formula I or as defined in embodiment (1). In another embodiment, the Compound is that where R⁶ is located in the meta position of the phenyl ring to which it is attached; R⁶ is —S(O)₂R⁸; R⁸ is alkyl.

### Embodiments (J)

In another embodiment, the Compound is according to Formula I(h)

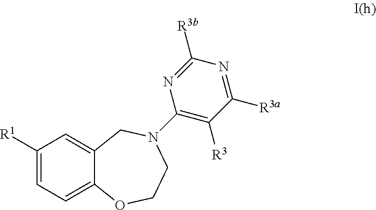

where R¹, R³, R^{3a}, and R^{3b} are independently as defined in the Summary of the Invention for a Compound of Formula I or as defined in embodiment (1). In another embodiment, the Compound of Formula I(h) is that where R³, R^{3a}, and R^{3b} are as described in any of embodiments (D3a)-(D3c), (D3g), and (D3i); and all other groups are as defined in the Summary of the Invention for a Compound of Formula I or as defined in embodiment (1).

In another embodiment of embodiments (J), the Compound of Formula I(h) is that where R¹ is according to any of embodiments (Z)-(Z5); and all other groups are as defined in the Summary of the Invention for a Compound of Formula I or as defined in embodiment (1).

In another embodiment of embodiments (K), the Compound of Formula I is according to Formula I(j)

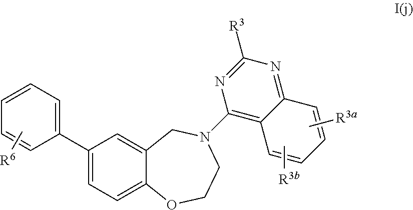

where R³, R^{3a}, R^{3b}, and R⁶ are independently as defined in the Summary of the Invention for a Compound of Formula I or as defined in embodiment (1). In another embodiment, the Compound is of Formula I(j) where R³, R^{3a}, and R^{3b} are as defined in embodiments (E2b); and all other groups are as defined in the Summary of the Invention for a Compound of Formula I or as defined in embodiment (1). In another embodiment, the Compound is of Formula I(j) where R³ is hydrogen, alkyl (in another embodiment alkyl is C₁₋₃-alkyl), halo, —OR^{11a}, or alkyl substituted with one R¹⁶; R³ is hydrogen; R^{3a} is hydrogen or alkoxy; and R⁶ is as defined in the Summary of the Invention for a Compound of Formula I or as defined in embodiment (1).

In another embodiment of embodiments (K), the Compound of Formula I(j) is that where R⁶ is according to embodiments (X); and all other groups are as defined in the Summary of the Invention for a Compound of Formula I or as defined in embodiment (1).

In another embodiment of embodiments (L), the Compound of Formula I is according to Formula I(k)

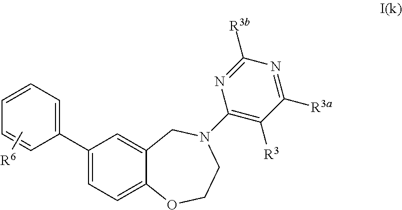

where R³, R^{3a}, R^{3b}, and R⁶ are independently as defined in the Summary of the Invention for a Compound of Formula I or as defined in embodiment (1). In another embodiment, the Compound of Formula I(h) is that where R³, R^{3a}, and R^{3b} are as described in any of embodiments (D3a)-(D3c), (D3g), and (D3i); and all other groups are as defined in the Summary of the Invention for a Compound of Formula I or as defined in embodiment (1).

In another embodiment of embodiments (L), the Compound of Formula I(k) is that where R⁶ is according to embodiments (X); and all other groups are as defined in the Summary of the Invention for a Compound of Formula I or as defined in embodiment (1).

In another embodiment of embodiments (M), the Compound of Formula I is according to Formula I(m)

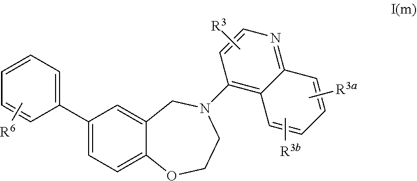

where R³, R^{3a}, R^{3b}, and R⁶ are independently as defined in the Summary of the Invention for a Compound of Formula I or as defined in embodiment (1). In another embodiment, the Compound is of Formula I(m) where R³ is hydrogen, alkyl (in another embodiment alkyl is C₁₋₃-alkyl), or alkyl substituted with one R¹⁶, —OR^{11a}; R^{3a} is hydrogen or —OR^{11a}; and R^{3b} is hydrogen or alkyl; and R⁶ is as defined in the Summary of the Invention for a Compound of Formula I or as defined in embodiment (1). In another embodiment, the Compound is of Formula I(m) where R³, R^{3a}, and R^{3b} are as defined in embodiments (E6a); and R⁶ is as defined in the Summary of the Invention for a Compound of Formula I or as defined in embodiment (1).

In another embodiment of embodiments (M), the Compound of Formula I(m) is that where R⁶ is according to embodiments (X); and all other groups are as defined in the Summary of the Invention for a Compound of Formula I or as defined in embodiment (1).

In another embodiment of embodiments (N), the Compound is of Formula I(n)

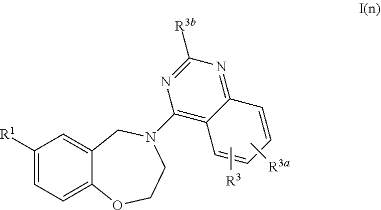

where R¹ is as defined in the Summary of the Invention for a Compound of Formula I or as defined in embodiment (1); and one of R³, R^{3a}, and R^{3b} and all other groups are independently as defined in the Summary of the Invention for a Compound of Formula I or as defined in embodiment (1). In another embodiment of embodiments (N), the Compound is of Formula I(n) where R³, R^{3a}, R^{3b}, and R¹ are independently as defined in the Summary of the Invention for a Compound of Formula I or as defined in embodiment (1). In another embodiment, the Compound is of Formula I(n) where R³, R^{3a}, and R^{3b} is as defined in embodiments (E2b); and all other groups are as defined in the Summary of the Invention for a Compound of Formula I or as defined in embodiment (1). In another embodiment, the Compound is of Formula I(n) where R³ is hydrogen, alkyl (in another embodiment alkyl is C₁₋₃-alkyl), halo, —OR^{11a}, or alkyl substituted with one R¹⁶; R³ is hydrogen; R^{3a} is hydrogen or alkoxy; and R¹ is as defined in the Summary of the Invention for a Compound of Formula I or as defined in embodiment (1). In another embodiment, the Compound is of Formula I(n) where R¹ is as defined in the Summary of the Invention for a Compound of Formula I or as defined in embodiment (1); and two of R³, R^{3a}, and R^{3b} are hydrogen and the others are independently as defined in the Summary of the Invention for a Compound of Formula I or as defined in embodiment (1). In another embodiment, the Compound is of Formula I(n) where R¹ is as defined in the Summary of the Invention for a Compound of Formula I or as defined in embodiment (1); and three of R³, R^{3a}, and R^{3b} are hydrogen and the others are independently as defined in the Summary of the Invention for a Compound of Formula I or as defined in embodiment (1).

In another embodiment of embodiments (N), the Compound of Formula I(n) is that where R¹ is according to any of embodiments (Z)-(Z5); and all other groups are as defined in the Summary of the Invention for a Compound of Formula I or as defined in embodiment (1).

### Embodiments (P)

In another embodiment, the Compound is of Formula I(p)

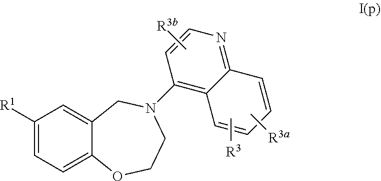

where R¹ is as defined in the Summary of the Invention for a Compound of Formula I; and one of R³, R^{3a}, and R^{3b} is hydrogen and the others are independently as defined in the Summary of the Invention for a Compound of Formula I. In another embodiment, the Compound is of Formula I(p) where R¹ is as defined in the Summary of the Invention for a Compound of Formula I; and one of R³, R^{3a}, and R^{3b} are hydrogen and the others are independently as defined in the Summary of the Invention for a Compound of Formula I. In another embodiment, the Compound is of Formula I(p) where R¹ is as defined in the Summary of the Invention for a Compound of Formula I; and two of R³, R^{3a}, and R^{3b} are hydrogen and the others are independently as defined in the Summary of the Invention for a Compound of Formula I. In another embodiment, the Compound is of Formula I(p) where R³ is hydrogen, alkyl (in another embodiment alkyl is C₁₋₃-alkyl), or alkyl substituted with one R¹⁶, —OR^{11a}; R^{3a} is hydrogen or —OR^{11a}; and R^{3b} is hydrogen or alkyl; and R⁶ is as defined in the Summary of the Invention for a Compound of Formula I or as defined in embodiment (1). In another embodiment, the Compound is of Formula I(p) where R³, R^{3a}, and R^{3b} are as defined in embodiments (E6a); and R⁶ is as defined in the Summary of the Invention for a Compound of Formula I or as defined in embodiment (1).

In another embodiment of embodiments (P), the Compound of Formula I(p) is that where R¹ is according to any of embodiments (Z)-(Z5); and all other groups are as defined in the Summary of the Invention for a Compound of Formula I or as defined in embodiment (1).

### Embodiments Q

In another embodiment, the Compound is of Formula I(q)

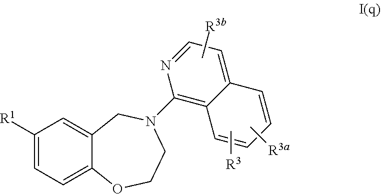

where R¹ is as defined in the Summary of the Invention for a Compound of Formula I; and one of R³, R^{3a}, and R^{3b} is hydrogen and the others are independently as defined in the Summary of the Invention for a Compound of Formula I. In another embodiment, the Compound is of Formula I(q) where R¹ is as defined in the Summary of the Invention for a Compound of Formula I; and two of R³, R^{3a}, and R^{3b} are hydrogen and the others are independently as defined in the Summary of the Invention for a Compound of Formula I. In another embodiment, the Compound is of Formula I(q) where R¹ is as defined in the Summary of the Invention for a Compound of Formula I; and three of R³, R^{3a}, and R^{3b} are hydrogen and the others are independently as defined in the Summary of the Invention for a Compound of Formula I.

In another embodiment of embodiments (O), the Compound of Formula I(q) is that where R¹ is according to any of embodiments (Z)-(Z5); and all other groups are as defined in the Summary of the Invention for a Compound of Formula I or as defined in embodiment (1).

### Embodiment (F)

In another embodiment, the Compound is of Formula I(r)

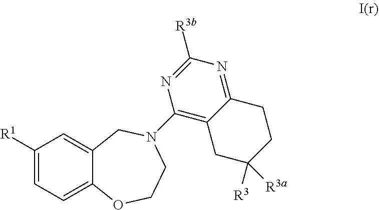

where R¹, R³, R^{3a}, and R^{3b} are independently as defined in the Summary of the Invention for a Compound of Formula I. In another embodiment, the Compound of Formula I(r) is where R³ and R^{3a} are alkyl (in another embodiment alkyl is C₁₋₃-alkyl) and R^{3b} is hydrogen, alkyl (in another embodiment alkyl is C₁₋₃-alkyl), haloalkyl, or alkyl substituted with one R¹⁶; and all other groups are as defined in the Summary of the Invention for a Compound of Formula I or as defined in embodiment (1). In another embodiment, the Compound of Formula I(r) is where R³ and R^{3a} are halo and R^{3b} is hydrogen, alkyl (in another embodiment alkyl is C₁₋₃-alkyl), haloalkyl, or alkyl substituted with one R¹⁶; and all other groups are as defined in the Summary of the Invention for a Compound of Formula I or as defined in embodiment (1). In another embodiment, the Compound of Formula I(r) is where R³ and R^{3a} together with the carbon to which they are attached form an optionally substituted cycloalkyl and R^{3b} is hydrogen, alkyl (in another embodiment alkyl is C₁₋₃-alkyl), haloalkyl, or alkyl substituted with one R¹⁶; and all other groups are as defined in the Summary of the Invention for a Compound of Formula I or as defined in embodiment (1).

In another embodiment of embodiments (F), the Compound of Formula I(r) is that where R¹ is according to any of embodiments (Z)-(Z5); and all other groups are as defined in the Summary of the Invention for a Compound of Formula I or as defined in embodiment (1).

### Embodiments (S)

In another embodiment, the Compound is of Formula I(s)

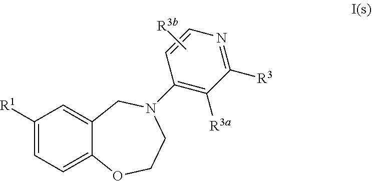

where R³ is cyano, alkyl (in another embodiment alkyl is C₁₋₃-alkyl), halo, haloalkyl, —SR¹², alkylsulfonyl, optionally substituted phenyl, optionally substituted phenylalkyl, optionally substituted cycloalkyl, optionally substituted cycloalkylalkyl, carboxy, —C(O)OR⁴, —NR¹¹R^{11a}, or —OR^{11a}; and R¹, R^{3a}, R^{3b}, R⁴, R¹¹, and R^{11a} are independently as defined in the Summary of the Invention for a Compound of Formula I.

In another embodiment of embodiments (S), the Compound of Formula I(s) is that where R¹ is according to any of embodiments (Z)-(Z5); and all other groups are as defined in the Summary of the Invention for a Compound of Formula I or as defined in embodiment (1).

### Embodiments (T)

In another embodiment, the Compound is of Formula I(t)

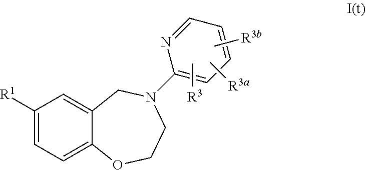

where R¹, R³, R^{3a}, and R^{3b} are independently as defined in the Summary of the Invention for a Compound of Formula I.

In another embodiment of embodiments (T), the Compound of Formula I(t) is that where R¹ is according to any of embodiments (Z)-(Z5); and all other groups are as defined in the Summary of the Invention for a Compound of Formula I or as defined in embodiment (1).

### Embodiment (U)

In another embodiment, the Compound is according to Formula I(a) where R¹ is heteroaryl optionally substituted with one or two R⁷; each R⁷, when present, is independently halo, alkyl, cycloalkyl, haloalkyl, hydroxyalkyl, alkoxyalkyl, —NR⁸R^{8a}, or —NR⁸C(O)OR⁹; and all other groups are independently as defined in the Summary of the Invention for a Compound of Formula I. In another embodiment, the Compound is according to Formula I(a) where R¹ is heteroaryl optionally substituted with one or two R⁷; each R⁷, when present, is independently alkyl (in another embodiment alkyl is C₁₋₃-alkyl), cycloalkyl, haloalkyl, —NR⁸R^{8a}, or —NR⁸C(O)OR⁹; and all other groups are independently as defined in the Summary of the Invention for a Compound of Formula I. In another embodiment, the Compound is according to Formula I(a) where R¹ is heteroaryl optionally substituted with one or two R⁷; each R⁷, when present, is independently alkyl (in another embodiment alkyl is C₁₋₃-alkyl), cycloalkyl, haloalkyl, —NR⁸R^{8a}, or —NR⁸C(O)OR⁹; R⁸ is hydrogen; R^{8a} is hydrogen, alkyl (in another embodiment alkyl is C₁₋₃-alkyl), or haloalkyl; and R⁹ is hydrogen or alkyl (in another embodiment alkyl is C₁₋₃-alkyl); and all other groups are independently as defined in the Summary of the Invention for a Compound of Formula I.

In another embodiment, the Compound is according to Formula I(a) where R² is 5,6,7,8-tetrahydroquinolin-4-yl or 5,6,7,8-tetrahydroisoquinolin-1-yl, where R² is substituted with R³, R^{3a}, R^{3b}, R^{3c}, and R^{3d}; and R¹, R³, R^{3a}, R^{3b}, R^{3c}, and R^{3d} are independently as defined in the Summary of the Invention for a Compound of Formula I. In another embodiment, the Compound is according to Formula I(a) where R² is 5,6,7,8-tetrahydroquinolin-4-yl or 5,6,7,8-tetrahydroisoquinolin-1-yl, where R² is substituted with R³, R^{3a}, R^{3b}, R^{3c}, and R^{3d}; R^{3d} is hydrogen; and R¹, R³, R^{3a}, R^{3b}, and R^{3c} are independently as defined in the Summary of the Invention for a Compound of Formula I. In another embodiment, the Compound is according to Formula I(a) where R² is 5,6,7,8-tetrahydroquinolin-4-yl or 5,6,7,8-tetrahydroisoquinolin-1-yl, where R² is substituted with R³, R^{3a}, R^{3b}, R^{3c}, and R^{3d}; R^{3b}, R^{3c}, and R^{3d} are hydrogen; and R¹, R³, and R^{3a} are independently as defined in the Summary of the Invention for a Compound of Formula I. In another embodiment, the Compound is according to Formula I(a) where R² is 5,6,7,8-tetrahydroquinolin-4-yl or 5,6,7,8-tetrahydroisoquinolin-1-yl, where R² is substituted with R³, R^{3a}, R^{3b}, R^{3a}, and R^{3d}; R^{3a}, R^{3b}, R^{3c}, and R^{3d} are hydrogen; and R¹, and R³ are independently as defined in the Summary of the Invention for a Compound of Formula I. In another embodiment, the Compound is according to Formula I(a) where R² is 5,6,7,8-tetrahydroquinolin-4-yl or 5,6,7,8-tetrahydroisoquinolin-1-yl, where R² is substituted with R³, R^{3a}, R^{3b}, R^{3c}, and R^{3d}; R³, R^{3a}, R^{3b}, R^{3c}, and R^{3d} are hydrogen; and R¹ is as defined in the Summary of the Invention for a Compound of Formula I.

Another embodiment provides a pharmaceutical composition which comprises 1) a compound, as a single stereoisomer or mixture of stereoisomers thereof, according to any one of Formula I, (I(a), I(b1), I(b2), I(c1), I(c2), I(d1), I(d2), I(e), I(e1), I(f), I(g), I(h), I(j), I(k), I(m), I(n), I(p), I(q), I(r), I(s), and I(t) or according to any one of the above embodiments, optionally as a pharmaceutically acceptable salt thereof, and 2) a pharmaceutically acceptable carrier, excipient, and/or diluent thereof.

Another embodiment is a method of treating disease, disorder, or syndrome where the disease is associated with uncontrolled, abnormal, and/or unwanted cellular activities effected directly or indirectly by PI3K and/or mTOR which method comprises administering to a human in need thereof a therapeutically effective amount of a Compound of any of Formula I, (I(a), I(b1), I(b2), I(c1), I(c2), I(d1), I(d2), I(e), I(e1), I(t), I(g), I(h), I(j), I(k), I(m), I(n), I(p), I(q), I(r), I(s), and I(t), a Compound of any one of the above embodiments, or a Compound from Table 1, optionally as a pharmaceutically acceptable salt or pharmaceutical composition thereof. In another embodiment the disease is cancer. In another embodiment, the disease is cancer and the Compound is of Formula I(a) or a Compound from Table 1.

### Embodiment (G)

Another embodiment is directed to a method of treating a disease, disorder, or syndrome which method comprises administering to a patient a therapeutically effective amount of a Compound of any of Formula I, (I(a), I(b1), I(b2), I(c1), I(c2), I(d1), I(d2), I(e), I(e1), I(f), I(g), I(h), I(j), I(k), I(m), I(n), I(p), I(q), I(r), I(s), and I(t), a Compound of any one of the above embodiments, or a Compound from Table 1, optionally as a pharmaceutically acceptable salt thereof, or a pharmaceutical composition comprising a therapeutically effective amount of a Compound of Formula I, (I(a), I(b1), I(b2), I(c1), I(c2), I(d1), I(d2), I(e), gel), I(e1), I(g), I(h), I(j), I(k), I(m), I(n), I(p), I(q), I(r), I(s), and I(t), a Compound of any one of the above embodiments, or a Compound from Table 1, and a pharmaceutically acceptable carrier, excipient, or diluent. In another embodiment the disease is cancer.

In another embodiment of any of the embodiments of Embodiment (G), the cancer is breast cancer, mantle cell lymphoma, renal cell carcinoma, acute myelogenous leukemia, chronic myelogenous leukemia, NPM/ALK-transformed anaplastic large cell lymphoma, diffuse large B cell lymphoma, rhabdomyosarcoma, ovarian cancer, endometrial cancer, cervical cancer, non small cell lung carcinoma, small cell lung carcinoma, adenocarcinoma, colon cancer, rectal cancer, gastric carcinoma, hepatocellular carcinoma, melanoma, pancreatic cancer, prostate carcinoma, thyroid carcinoma, anaplastic large cell lymphoma, hemangioma, glioblastoma, or head and neck cancer.

All Compounds in Table 1 were tested in the assays described in Biological Examples 1 and 3.

### Embodiments (V)

In one embodiment the Compound of the Invention has an PI3K-alpha-inhibitory activity of about 2.0 μM or less and is inactive for mTOR (when tested at a concentration of 3.0 μM or greater) or is selective for PI3K-alpha over mTOR by about 5-fold or greater, about 7-fold or greater, or about 10-fold or greater. In another embodiment the Compound of the Invention has an PI3K-alpha-inhibitory activity of about 1.0 μM or less and is inactive for mTOR (when tested at a concentration of 2.0 μM or greater) or is selective for PI3K-alpha over mTOR by about 5-fold or greater, about 7-fold or greater, or about 10-fold or greater. In another embodiment the Compound of the Invention has an PI3K-alpha-inhibitory activity of about 0.5 μM or less and is inactive for mTOR (when tested at a concentration of 2.0 μM or greater) or is selective for PI3K-alpha over mTOR by about 5-fold or greater, about 7-fold or greater, or about 10-fold or greater. In another embodiment the Compound of the Invention has an PI3K-alpha-inhibitory activity of about 0.3 μM or less and is inactive for mTOR (when tested at a concentration of 2.0 μM or greater) or is selective for PI3K-alpha over mTOR by about 5-fold or greater, about 7-fold or greater, or about 10-fold or greater. In another embodiment the Compound of the Invention has an PI3K-alpha-inhibitory activity of about 0.2 μM or less and is selective for PI3K-alpha over mTOR by about 5-fold or greater, about 7-fold or greater, or about 10-fold or greater. In another embodiment the Compound of the Invention has an PI3K-alpha-inhibitory activity of about 0.1 μM or less and is selective for PI3K-alpha over mTOR by about 5-fold or greater, about 7-fold or greater, or about 10-fold or greater. In another embodiment the Compound of the Invention has an PI3K-alpha-inhibitory activity of about 0.05 μM or less and is selective for PI3K-alpha over mTOR by about 5-fold or greater, about 7-fold or greater, or about 10-fold or greater. In another embodiment the Compound of the Invention has an PI3K-alpha-inhibitory activity of about 0.025 μM or less and is selective for PI3K-alpha over mTOR by about 5-fold or greater, about 7-fold or greater, or about 10-fold or greater. In another embodiment the Compound of the Invention has an PI3K-alpha-inhibitory activity of about 0.01 μM or less and is selective for PI3K-alpha over mTOR by about 5-fold or greater, about 7-fold or greater, or about 10-fold or greater.

### Embodiments (W)

In one embodiment the Compound of the Invention has an PI3K-alpha-inhibitory activity of about 2.0 μM or less and an mTOR-inhibitory activity of about 2.0 μM or less and the selectivity for one of the targets over the other does not exceed 3-fold. In another embodiment the Compound of the Invention has an PI3K-alpha-inhibitory activity of about 1.0 μM or less and an mTOR-inhibitory activity of about 1.0 μM or less and the selectivity for one of the targets over the other does not exceed 3-fold. In another embodiment the Compound of the Invention has an PI3K-alpha-inhibitory activity of about 0.5 μM or less and an mTOR-inhibitory activity of about 0.5 μM or less and the selectivity for one of the targets over the other does not exceed 3-fold. In another embodiment the Compound of the Invention has an PI3K-alpha-inhibitory activity of about 0.3 μM or less and an mTOR-inhibitory activity of about 0.3 μM or less and the selectivity for one of the targets over the other does not exceed 3-fold. In another embodiment the Compound of the Invention has an PI3K-alpha-inhibitory activity of about 0.15 μM or less and an mTOR-inhibitory activity of about 0.15 μM or less and the selectivity for one of the targets over the other does not exceed 2-fold. In another embodiment the Compound of the Invention has an PI3K-alpha-inhibitory activity of about 0.1 μM or less and an mTOR-inhibitory activity of about 0.1 μM or less. In another embodiment the Compound of the Invention has an PI3K-alpha-inhibitory activity of about 0.05 μM or less and an mTOR-inhibitory activity of about 0.05 μM or less. In another embodiment the Compound of the Invention has an PI3K-alpha-inhibitory activity of about 0.02 μM or less and an mTOR-inhibitory activity of about 0.02 μM or less. In another embodiment the Compound of the Invention has an PI3K-alpha-inhibitory activity of about 0.01 μM or less and an mTOR-inhibitory activity of about 0.01 μM or less.

In another embodiment, Compounds of the invention are also useful as inhibitors of PI3Kα and/or mTOR in vivo for studying the in vivo role of PI3Kα and/or mTOR in biological processes, including the diseases described herein. Accordingly, the invention also comprises a method of inhibiting PI3Kα and/or mTOR in vivo comprising administering a compound or composition of the invention to a mammal.

### Embodiment (X)

Another embodiment is directed to a therapeutic method for treating a subject having a tumor. Phosphatidylinositol 3-kinases (PI 3-kinases or PI3Ks) are a family of enzymes involved in cellular functions such as cell growth, proliferation, differentiation, motility, survival and intracellular trafficking, which in turn are involved in cancer. PI3Ks are a family of related intracellular signal transducer enzymes capable of phosphorylating the 3 position hydroxyl group of the inositol ring of phosphatidylinositol (PtdIns). Phosphatidylinositol 3-kinase is composed of an 85 kDa regulatory subunit and a 110 kDa catalytic subunit. The protein encoded by PI3KCA gene represents the catalytic subunit, which uses ATP to phosphorylate phosphatidylinositols (PtdIns), PtdIns4P and PtdIns(4,5)P2.

In the present invention, reference to position within the amino acid sequence of PI3Kα is made referring to SEQ ID NO: 1. Reference to positions within the nucleotide sequence of the PI3Kα is made referring to SEQ ID NO:2. Specific amino acids in the wild type protein sequence are described using single letter amino acid designation followed by the position in the protein sequence, for example E545 indicates that position 545 is glutamic acid. To represent a substitution at a particular position, the substituted amino acid follows the position, for example E545K indicates that the glutamic acid at position 545 is replaced with a lysine.

As used herein, the term “subject” refers to a mammal, preferably a human mammal, that can be afflicted by a cancer disease. Typically, the terms “subject” and “patient” are used herein interchangeably in reference to a human individual. In various embodiments, reference to human PI3K-α in the various methods and description of genetic variants herein refers to the human PI3K-p110α catalytic subunit. In some embodiments a gene which encodes an exemplary PI3K-α is illustrated in GenBank Accession No. NG 012113 located on chromosome 3 at map coordinates 3q26.3. Other synonyms include: MGC142161; MGC142163; p110-alpha; PI3Kα.

In one illustrative embodiment, a mature PI3K-α protein sequence is encoded by a mRNA (NCBI Accession No. NM 006218, version NM 006218.2 G1: 54792081.)

Activation of PI3K signaling occurs in the majority of human cancers. Mechanisms of pathway dysregulation include over-expression or mutational activation of upstream receptor tyrosine kinases or components of the pathway including PI3K-α and inactivation the lipid phosphatase PTEN. Mutations in PI3K-α occur most frequently at hotspots in the helical domain (E545K) or kinase domain (H1047R). The effects of different PI3K pathway-activating genetic lesions are not equivalent. PTEN-null tumor cells demonstrate high basal pAKT levels while PI3K-α mutant cells are either RAS-dependent with low basal levels of pAKT (E545K) or RAS-independent with more variable levels of pAKT (H1047R) (Vasudevan, 2009; Zhao, 2008; Mandelker, 2009; Pang, 2009).

The high frequency of PI3K pathway activation in human tumors has lead to the development of PI3K inhibitors as cancer therapeutics. First generation compounds are largely pan-PI3K inhibitors that target more than one class I PI3K isoform (PI3K-α, PI3Kβ, PI3Kδ, and PI3Kγ) or related protein kinases such as mTOR. In order to identify genetic lesions which sensitize cells to PI3K inhibitors, investigators have profiled panels of tumor cell lines using protein phosphorylation or cell growth/viability as readouts. In general, mutational activation of PI3K-α, lack of PTEN function, and HER2 over-expression were found to sensitize cells to pan-PI3K compounds while mutational activation of KRAS lead to desensitization (Serra, 2008; Brachmann, 2009; O'Brien, 2010).

Despite increased attention, the genetic backgrounds where inhibition of specific PI3K isozymes would block cell signaling and growth and provide therapeutic benefit are less well defined. For PTEN-negative tumors, specific inhibition of PI3Kβ (but not PI3Kα) using RNAi or selective inhibitors (e.g. TGX-221, EXEL-04214154) blocks basal AKT phosphorylation and cell growth (Wee, 2008, Edgar, 2010). For PI3K-α mutant/PTEN positive tumors, inhibition of PI3Kβ has little effect while PI3Kα knockdown by siRNA reduces basal AKT phosphorylation and cell growth (Wee, 2009), however differential effects in H1047R vs. E545K cells have not been reported.

In this regard, a therapeutic method for treating a subject having a tumor comprises: (a) administering a PI3K-α selective inhibitor, a dual PI3K-α/mTOR selective inhibitor, or a combination of a PI3K-α selective inhibitor and a mTOR selective inhibitor to the subject if said tumor comprises a mutation in a PI3K-α kinase domain; or (b) administering a combination of a PI3K-α selective inhibitor and a PI3K-β selective inhibitor, a dual PI3K-α/mTOR selective inhibitor, or a PI3K-β selective inhibitor, to said subject if said tumor comprises a mutation in a PI3K-α helical domain.

In some embodiments, the tumors being treated can include one or more of a breast cancer, a mantle cell lymphoma, a renal cell carcinoma, an acute myelogenous leukemia, a chronic myelogenous leukemia, a NPM/ALK-transformed anaplastic large cell lymphoma, a diffuse large B cell lymphoma, a rhabdomyosarcoma, an ovarian cancer, an endometrial cancer, a cervical cancer, a non-small cell lung carcinoma, a small-cell lung carcinoma, a melanoma, a pancreatic cancer, a prostate carcinoma, a thyroid carcinoma, an anaplastic large cell lymphoma, a hemangioma, a glioblastoma, or a head and neck cancer.

In some embodiments of the present invention, the therapeutic method first requires a tumor sample from the subject, wherein the sample can be any tumor tissue sample that is believed to contain a tumor cell. In some embodiments, subjects in need of a cancer treatment have often been diagnosed as having a tumor or cancer and samples of such tumor or cancer can be readily obtained using standard oncological methods known in the art. In some embodiments, the tumor cell obtained from the patient can be obtained using laparoscopic, endoscopic or surgical means, for example, a direct incision into a tumor mass as located and/or identified using any screening means, for example, direct palpation, radiographic or tomographic means, e.g. MRI or CT/PET Scans.

In some embodiments, the tumor cells can be cultured from a biopsied tissue for further screening assays or other methods described herein. For example, >100 mg of non-necrotic, non-contaminated tissue can harvested from the patient by any suitable biopsy or surgical procedure known in the art. Biopsy sample preparation can generally proceed under sterile conditions, for example, under a Laminar Flow Hood which should be turned on at least 20 minutes before use. Reagent grade ethanol is used to wipe down the surface of the hood prior to beginning the sample preparation. The tumor is then removed, under sterile conditions, from the shipping container and is minced with sterile scissors. If the specimen arrives already minced, the individual tumor pieces should be divided into groups. Using sterile forceps, each undivided tissue section is then placed in 3 ml sterile growth medium (Standard F-10 medium containing 17% calf serum and a standard amount of Penicillin and Streptomycin) and systematically minced by using two sterile scalpels in a scissor-like motion, or mechanically equivalent manual or automated opposing incisor blades. This cross-cutting motion is important because the technique creates smooth cut edges on the resulting tumor multicellular particulates. Preferably but not necessarily, the tumor particulates each measure 1 mm³. After each tumor quarter has been minced, the particles are plated in culture flasks using sterile pasteur pipettes (9 explants per T-25 or 20 particulates per T-75 flask). Each flask is then labeled with the patient's code, the date of explantation and any other distinguishing data.

The explants can be evenly distributed across the bottom surface of the flask, with initial inverted incubation in a 37° C. incubator for 5-10 minutes, followed by addition of about 5-10 mL sterile growth medium and further incubation in the normal, non-inverted position. Flasks are placed in a 35° C., non-CO₂ incubator. Flasks should be checked daily for growth and contamination. Over a period of a few weeks, with weekly removal and replacement of 5 ml of growth medium, the explants will foster growth of cells into a monolayer. With respect to the culturing of tumor cells, (without wishing to be bound by any particular theory) maintaining the malignant cells within a multicellular particulate of the originating tissue, growth of the tumor cells themselves is facilitated versus the overgrowth of fibroblasts (or other unwanted cells) which tends to occur when suspended tumor cells are grown in culture.

The use of the above procedure to form a cell monolayer culture maximizes the growth of malignant cells from the tissue sample, and thus optimizes ensuing tissue culture assay of chemotherapeutic action of various agents to be tested. Enhanced growth of actual malignant cells is only one aspect of the present invention, however; another important feature is the growth rate monitoring system used to oversee growth of the monolayer once formed. Once a primary culture and its derived secondary monolayer tissue culture has been initiated, the growth of the cells is monitored to ascertain the time to initiate the chemotherapy assay and to determine the growth rate of the cultured cells.

The tumor cell whether a primary tumor cell or cultured tumor cell from the subject's tumor can then be interrogated to determine whether the isolated tumor cell from the subject contains a mutation in the kinase domain or in the helical domain of PI3K-α. Once the sequencing data for each sample of nucleic acid has been confirmed, the sequence itself can be read to determine whether or not the tumor cell has a mutation in a kinase domain and/or the helical domain of PI3K-α. In some embodiments, the nucleotide sequence can be converted into a protein amino acid sequence of a mature, full length PI3K p110-α subunit or fragment thereof containing the amino acids representative of the diagnostic mutations described herein. Purely for the purposes of the present application, the PI3KCA or PI3K p110-α catalytic subunit is herein referred to as PI3K-α. The designation of such should not be confused with the regulatory p85-α subunit.

While several embodiments herein have exemplified human PI3K-α, other PI3K-α subunit encoding nucleotides and full length amino acid sequences are readily available from depository of bioinformatic databases such as NCBI, UniProtKB-Swiss-Prot and TrEMBL−, UniRef, UniParc and the like. In some embodiments, the methods to identify a protein sequence of PI3K-α can employ a nucleic acid-based approach or a protein based approach. In both respects, the determination of whether a mutation in PI3K-α kinase domain or catalytic domain can be readily performed using assays that are well known in the field of identifying genetic mutations. As used herein for exemplary purposes only, full length human PI3K-α is exemplified in SEQ ID NO:1.

As used herein, the kinase domain of a human PI3K-α (PI3KCA) includes the kinase domain located in axon 20 which spans approximately from amino acid 699-1064 of SEQ ID NO: 1. In some embodiments, the methods of the present invention identifies whether the subject's tumor cell has a mutation at position 1047. In some embodiments, the mutation in the kinase domain includes a substitution of histidine to arginine at position 1047 of SEQ ID NO: 1.

In some embodiments, once the subject has been identified as having a tumor cell with a mutation in the kinase domain, for example, a mutation at amino acid 1047 of SEQ ID NO:1, the patient can be administered with a PI3K-α selective inhibitor. If the subject contains a mutation wherein histidine (H) is replaced with arginine (R) at position 1047 of SEQ ID NO:1, the subject is administered with a PI3K-α selective inhibitor, a dual PI3K-α/mTOR selective inhibitor, a combination of a PI3K-α selective inhibitor or a mTOR selective inhibitor

In some embodiments, a mutation of the helical domain in a subject's tumor cell can be used as a basis to treat the subject's tumor with a composition that does not include a PI3K-α selective inhibitor alone. As used herein, the helical domain refers to a domain in PI3K-α that span approximately from amino acid 526 to 696 of SEQ ID NO: 1. In some embodiments, the mutation to the helical domain can include a mutation to E542 of SEQ ID NO:1 mutating to E542K. In another embodiment, the mutation to the helical domain can include a mutation to E545 mutating to E545K. Exemplary mutations in the helical domain can include a mutation at position 542 and/or 545 of SEQ ID NO:1.

In some embodiments, the subject's tumor cell or cells have been used in one or more assays to determine the amino acid sequence directly or from sequence information obtained from nucleic acids encoding the PI3K-α. If the subject's tumor cell contains a mutation in the helical domain, the subject can be administered with one or more of a PI3K-α selective inhibitor and a PI3K-β selective inhibitor, a dual PI3K-α/mTOR selective inhibitor, or a PI3K-β selective inhibitor.

In some embodiments, PI3K-α selective inhibitors, dual PI3K-α/mTOR selective inhibitors and mTOR inhibitors can be selected from Table 1 below. In some embodiments, PI3K-α selective inhibitors, dual PI3K-α/mTOR selective inhibitors and mTOR inhibitors useful in the present inventive methods described in embodiments (X), (Y) and (Z) infra include those disclosed in International Patent Application Nos. PCT/US2006/039574 filed Oct. 9, 2006 and PCT/US2006/039734 filed Oct. 9, 2006. Both of these International Patent Applications are incorporated herein by reference in their entireties.

In some embodiments, a dual PI3K-α/mTOR selective inhibitor can include any one of:

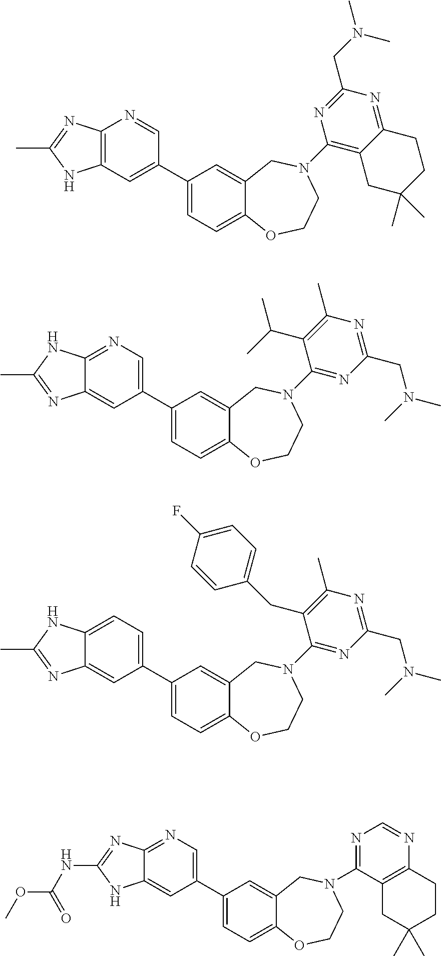

In some embodiments, the PI3K-α selective inhibitor can include any one of the following PI3K-α selective inhibitor compounds:

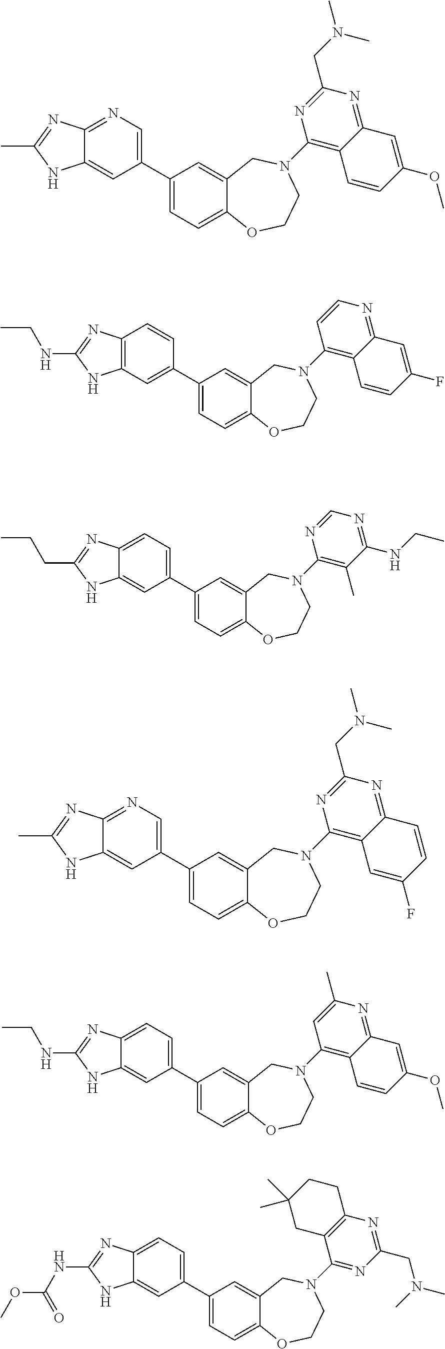

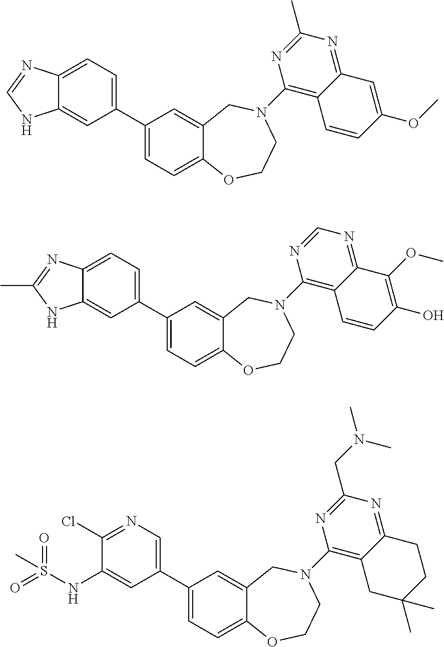

In some embodiments, a PI3K-α selective inhibitor includes:

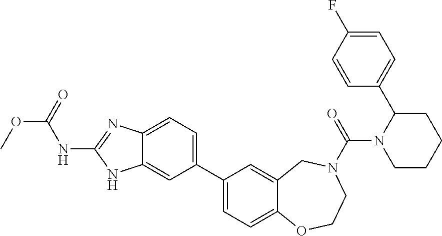 [SMILES: COC(=O)Nc1nc2ccc(-c3ccc4c(c3)CN(C(=O)N3CCCCC3c3ccc(F)cc3)CCO4)cc2[nH]1]

In various embodiments of the present invention, the variously described inhibitors can be administered to a subject having a tumor or cancer in pharmaceutical compositions according to the invention. The pharmaceutical composition can include a PI3K-α selective inhibitor, a dual PI3K-α/mTOR selective inhibitor, a combination of a PI3K-α selective inhibitor or a mTOR selective inhibitor if the subject's tumor comprises a mutation in a PI3K-α kinase domain; or a PI3K-α selective inhibitor and a PI3K-β selective inhibitor, a dual PI3K-α/mTOR selective inhibitor, or a PI3K-β selective inhibitor, to the subject if the subject's tumor comprises a mutation in a PI3K-α helical domain. Each of these inhibitors can also include a pharmaceutically acceptable carrier, excipient, or diluent. In certain other specific embodiments, administration is by the oral route. Administration of the compounds of the invention, or their pharmaceutically acceptable salts, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as for example, tablets, suppositories, pills, soft elastic and hard gelatin capsules, powders, solutions, suspensions, or aerosols, or the like, specifically in unit dosage forms suitable for simple administration of precise dosages.

The compositions will include a conventional pharmaceutical carrier or excipient and a compound of the invention as the/an active agent, and, in addition, may include pharmaceutically acceptable carriers and adjuvants, etc can be administered in tablet, capsule, liquid, powder, nutritional bar or effervescent form. “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Methods of preparation of formulations for various forms of administration are known in the art and discussed in detail in Remington's Pharmaceutical Sciences, Eighteenth Edition (1990), incorporated herein by reference.

Dosages of the pharmaceutical composition of the present invention necessary to achieve a therapeutically effect may depend upon several factors. A “therapeutically effective amount” of a compound of the disclosed invention is the quantity which, when administered to a subject having a disease or disorder, results in regression of the disease or disorder or symptoms thereof, optionally including reduction in adverse side-effects in the subject when compared to another similarly prescribed medicine.

The amount of the disclosed compound to be administered to a subject will depend on the particular disorder, the mode of administration, co-administered compounds, if any, and the characteristics of the subject, such as general health, other diseases, age, sex, genotype, body weight and tolerance to drugs. The skilled artisan wilt be able to determine appropriate dosages depending on these and other factors. In some instances, a higher dosage is first prescribed to be titrated to a tolerable dose in which the subject does not experience overtly negative side effects which would result in cessation of treatment. In some embodiments, the dose of the compounds of the present invention can range in an amount of 0.001 mg/kg to about 100 mg/kg per day administered in single doses, multiple doses or in controlled release formulations. In some embodiments, therapeutically effective amounts of the disclosed compounds are administered typically in a range between about 0.01 mg/kg per day and about 50 mg/kg per day, and preferably between 0.1 mg/kg per day and about 10 mg/kg/day.

In some embodiments, the therapeutic method for treating a subject having a tumor can optionally comprise administering a compound of the present invention in addition to another chemotherapeutic agent. Among the many chemotherapeutic agents which may be used in combination with a compound of the present invention are anti-neoplastic agents. Anti-neoplastic agents may induce anti-neoplastic effects in a cell-cycle specific manner, i.e., are phase specific and act at a specific phase of the cell cycle, or bind DNA and act in a non cell-cycle specific manner, i.e., are non-cell cycle specific and operate by other mechanisms. Both types of anti-neoplastic agents may be employed in combination with the compounds of the present invention.

Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkyl sulfonates, nitrosoureas, and triazenes; antibiotic agents such as anthracyclines, actinomycins and bleomycins; topoisomerase H inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids. Examples of diterpenoids include, but are not limited to, paclitaxel and its analog docetaxel. Examples of vinca alkaloids include, but are not limited to, vinblastine, vincristine, and vinorelbine.

Platinum coordination complexes are non-phase specific anti-neoplastic agents, which are interactive with DNA. The platinum complexes enter tumor cells, undergo aquation and form intra- and interstrand crosslinks with DNA causing adverse biological effects to the tumor. Examples of platinum coordination complexes include, but are not limited to, cisplatin and carboplatin.

Alkylating agents are non-phase anti-neoplastic specific agents and strong electrophiles. Typically, alkylating agents form covalent linkages, by alkylation, to DNA through nucleophilic moieties of the DNA molecule such as phosphate, amino, and hydroxyl groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Antibiotic chemotherapeutic agents are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Topoisomerase II inhibitors include, but are not limited to, epipodophyllotoxins. Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mercaptopurine and thioguanine.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin.

Hormones and hormonal analogues are useful compounds for treating cancers in which there is a relationship between the hormone(s) and growth and/or lack of growth of the cancer. Examples of hormones and hormonal analogues believed to be useful in the treatment of neoplasms include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene and iodoxyfene useful in the treatment of hormone dependent breast carcinoma; and gonadotropin-releasing hormone (GnRH) and analogues thereof which stimulate the release of luteinizing hormone (LH) and/or follicle stimulating hormone (FSH) for the treatment prostatic carcinoma, for instance, LHRH agonists and antagonists such as goserelin acetate and luprolide.

Signal transduction pathway inhibitors are those inhibitors which block or inhibit a chemical process which evokes an intracellular change. As used herein this change is cell proliferation or differentiation or survival. Signal transduction inhibitors useful in the present invention include inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes. In some embodiments, the additional chemotherapeutic agent can include an inhibitor to other PI3K catalytic subunits (PI3K-β PI3K-δ, or PI3K-γ), or regulatory units, for example, TGX-221, or pan PI3K selective inhibitors, for example, PI-103 and PIK-75 at the dosages described above. As used herein TGX-221 ((CAS No. 663619-89-4) 7-methyl-2-(4-morpholinyl)-9-[1-(phenylamino)ethyl]-4H-pyrido[1,2-a]pyrimidin-4-one) is a potent, selective, and cell permeable inhibitor of PI3K p110β having the structure:

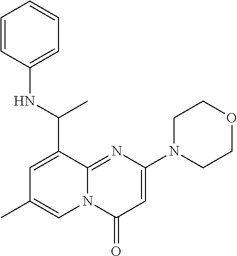 [SMILES: Cc1cc(C(C)Nc2ccccc2)c2nc(N3CCOCC3)cc(=O)n2c1]

and is commercially available from Cayman Chemicals, Catalog No. 10007349 (Ann Arbor, Mich., USA). As used herein, PI-103 ((CAS No. 371935-74-9) 3-[4-(4-morpholinyl)pyrido[3′,2′:4,5]furo[3,2-d]pyrimidin-2-yl]-phenol) is a cell-permeable, ATP-competitive inhibitor of phosphatidylinositol 3-kinase (PI3K) family members with selectivity toward DNA-PK, PI3K (p110α), and mTOR having the structure:

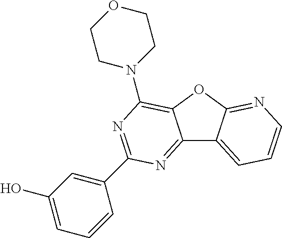 [SMILES: Oc1cccc(-c2nc(N3CCOCC3)c3oc4ncccc4c3n2)c1]

and is commercially available from Cayman Chemicals, Catalogue No. 10009209 (Ann Arbor Mich., USA). As used herein, PIK-75 ((CAS 372196-67-3), 2-methyl-5-nitro-2-[(6-bromoimidazo[1,2-a]pyridin-3-yl)methylene]-1-methylhydrazide-benzenesulfonic acid) is a PI3K kinase inhibitor having the structure:

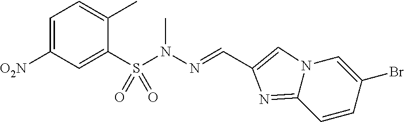 [SMILES: Cc1ccc([N+](=O)[O-])cc1S(=O)(=O)N(C)/N=C/c1cn2cc(Br)ccc2n1]

and is commercially available from Cayman Chemicals, Catalog No. 10009210 (Ann Arbor, Mich., USA).

### Embodiment (Y)

Another embodiment is directed to a method for identifying a selective inhibitor of a PI3K isozyme, the method comprising: (a) contacting a first cell bearing a first mutation in a PI3K-α with a candidate inhibitor; (b) contacting a second cell bearing a wild type PI3K-α, a PTEN null mutation, or a second mutation in said PI3K-α with the candidate inhibitor; and (c) measuring AKT phosphorylation in said first and said second cells, wherein decreased AKT phosphorylation in said first cell when compared to said second cell identifies said candidate inhibitor as a selective PI3K-α inhibitor.

As noted above, the newly discovered association between selective genetic mutations and increased sensitivities of some cancers to specific inhibitors renders a particular genetic background more susceptible to one or more types of inhibitors than others. This association between genetic backgrounds and susceptibilities of certain cancers provides an attractive and convenient cellular platform for identification of new selective inhibitors to PI3K kinases (e.g. via screening assays to detect compounds or entities that inhibit phosphorylation in a PI3K-α dependent manner). As will be appreciated by those of ordinary skill in the art, any kind of compounds or agents can be tested using the inventive screening methods. A candidate inhibitor compound may be a synthetic or natural compound; it may be a single molecule, a mixture of different molecules or a complex of at least two molecules. A candidate inhibitor can comprise functional groups necessary for structural interaction with proteins, particularly hydrogen bonding and lipophilic binding, and typically include at least an amine, carbonyl, hydroxyl, ether, or carboxyl group, for example at least two of the functional chemical groups. The candidate inhibitor often comprises cyclical carbon or heterocycloalkyl structures and/or aromatic or heteroaromatic structures substituted with one or more of the above functional groups. Candidate inhibitors are also found among biomolecules including peptides, saccharides, fatty acids, steroids, purines, pyrimidines, derivatives, structural analogs, or combinations thereof. In certain embodiments, the inventive methods are used for testing one or more candidate inhibitor compounds. In other embodiments, the inventive methods are used for screening collections or libraries of candidate inhibitor compounds. As used herein, the term “collection” refers to any set of compounds, molecules or agents, while the term “library” refers to any set of compounds, molecules or agents that are structural analogs.

Libraries of candidate inhibitor compounds that can be screened using the methods of the present invention may be either prepared or purchased from a number of companies. Synthetic compound libraries are commercially available from, for example, Comgenex (Princeton, N.J.), Brandon Associates (Merrimack, N.H.), Microsource (New Milford, Conn.), and Aldrich (Milwaukee, Wis.). Libraries of candidate inhibitor compounds have also been developed by and are commercially available from large chemical companies. Additionally, natural collections, synthetically produced libraries and compounds are readily modified through conventional chemical, physical, and biochemical means.

Cells to be used in the practice of the screening methods described herein may be primary cells, secondary cells, or immortalized cells (e.g., established cell lines). They may be prepared by techniques well known in the art (for example, cells may be obtained by fine needle biopsy from a patient or a healthy donor), available from research institutions and universities or purchased from immunological and microbiological commercial resources (for example, from the American Type Culture Collection (ATCC), Manassas, Va.). Alternatively or additionally, cells may be genetically engineered to contain, for example, a gene of interest, i.e. a cell line bearing a mutation in PI3K-α kinase domain, (for example, H1047R), and/or PI3K-α helical domain (for example, E542 and/or E545K). In some embodiments, in a first set of cells, the cells possess a genetic mutation in PI3K-α kinase domain, for example, H1047R. In a second set of cells to be used in the screening assays, the second set of cells possess a genetic mutation in a different kinase catalytic subunit, (for example, a mutation in a helical domain, for example, E545K, or in a different regulatory protein, for example Phosphatase and Tensin Homolog (PTEN). When a candidate inhibitor inhibits phosphorylation, (for example AKT phosphorylation) to a higher degree in the cell possessing the PI3K-α kinase domain genetic mutation when compared to a cell possessing a genetic mutation in a different kinase catalytic subunit, (for example a mutation in a helical domain, for example, E545K, or in a different regulatory protein), then the candidate inhibitor is a selective inhibitor for cancers or tumors that harbor activation mutations in PI3K-α. Conversely, PI3K-α-selective compounds inhibit AKT phosphorylation, PI3K pathway activation, and cell proliferation with greater potency in tumor cells harboring the PI3K-α-H1047R mutation compared to PTEN negative, PI3K-α wild-type, and PI3K-α-E545K backgrounds. Both PTEN inactivation and KRAS activation desensitize cells to the growth inhibitory effects of PI3K-α-selective compounds. A wild-type PI3K-α is illustratively provided in SEQ ID NO: 1 and is encoded by a mRNA of SEQ ID NO: 2.

In some embodiments, the first and second cells used in the screening assay have different genetic backgrounds. In one embodiment, the first cell group has a genetic mutation in a PI3K-α kinase domain. In an illustrative embodiment, the genetic mutation in the first cell group includes a mutation in a mRNA (NCBI Accession No. NM 006218, version NM 006218.2 GI: 54792081 herein disclosed as SEQ ID NO: 2 which encodes a full length PI3K-α having a mutation in the kinase domain. In one embodiment, an exemplary mutation is at a codon (3296, 3297 and 3298), in the kinase domain of SEQ ID NO: 2, wherein the codon is mutated to provide an amino acid other than a histidine at position 1047 of PI3K-α provided in SEQ ID NO: 1. In one exemplary mutation, the histidine at 1047 is mutated to arginine (H1047R). This mutation has been previously reported to be a particularly oncogenic mutation in the PI3K/AKT signaling pathway. The second cell group lacks the mutation of the first test cell group. In one embodiment, an exemplary mutation is at a codon (1790, 1791 and 1792), in the helical domain of SEQ ID NO: 2, wherein the codon is mutated to provide an amino acid other than a glutamic acid at position 545 of PI3K-α provided in SEQ ID NO: 1. In one exemplary mutation, the glutamic acid at 545 is mutated to lysine (E545K). This mutation has also been previously reported to be a particularly oncogenic mutation in the PI3K/AKT signaling pathway.

In some embodiments, the second cell group can harbor a mutation in PTEN.

In some embodiments, the first cell group can include various cell lines, including cancer cell lines, for example breast cancer cell lines that may be commercially available from the American Type Culture Collection ((ATCC) American Type Culture Collection, Manassas, Va.) bearing the H1047R het genetic mutation of PI3K-α. In some embodiments, the first cell can include HCT-116, T-47D, MDA-MB-453, SIGOV-3, BT-20 or LS H74T cell lines. In some embodiments, the second cell can include MCF-7, PC3 MCI-H460, SK-BR-3, PC-3, MDA-MB-468, SK-BR-3, MDA-MB-231T, or A549. Each specific cell line can be maintained according to instructions provided upon purchase and are commonly available through the ATCC. Table 3 in the examples section below provides exemplary first and second cell groups for use in the inventive methods described herein.

In some embodiments, the first cell group and second cell group can also include non-tumor cell lines that have been transformed with a mutant PI3K-α catalytic subunit, for example. H1047R het or E545K PI3K-α catalytic subunit. Methods of introducing nucleic acids and vectors into isolated cells and the culture and selection of transformed host cells in vitro are known in the art and include the use of calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, and electroporation (see, e.g., Sambrook et al., supra; Davis et al., Basic Methods in Molecular Biology, 2^{nd} ed., McGraw-Hill Professional, 1995; and Neumann et al., EMBO J., 1: 841 (1982)). There are several methods for eukaryotic cell transformation, either transiently or stably using a variety of expression vectors. Methods for mutating a cell-line, for example NIH 3T3 cells by amplifying a sequence of DNA encoding the mutated PI3K-α catalytic subunit of interest. The amplified PCR mutant PI3K-α construct can be cloned into a viral expression vector, for example, pSX2neo, a Moloney murine leukemia virus (MLV) long terminal repeat-driven expression vector made by inserting a simian virus 40 early promoter-neomycin phosphotransferase gene into pSX2, designed to express high levels of 10A1 MLV Env. Transformation of NIH 3T3 cells can be performed by transfection with a different CaPO₄ coprecipitation technique. After reaching confluence the cells can be transferred into a medium containing 5% FBS without dexamethasone. Morphologically transformed cells can be separated and isolated from mixtures of transformed and nontransformed Env-plasmid-transfected cells by excising the transformed foci from the cell layer with a small-bore pipette (a Pasteur pipette drawn out over a flame to give a fine tip) and aspiration of the foci by the use of a rubber bulb attached to a pipette.

In some embodiments, the methods described herein require that the cells be tested in the presence of a candidate inhibitor, wherein the candidate inhibitor is added to separate exemplary assay wells, each well containing either the first or second cells. The amount of candidate inhibitor can vary, such that a range of inhibitory activities can be determined for the determination of an IC₅₀ for that candidate inhibitor. This can easily be achieved by serially diluting the compound in an appropriate solvent, for example, DMSO and then in the culture medium in which the first and second cells are being incubated in. In some embodiments, the concentration of the candidate inhibitor can range from about 1 pM to about 1 mM concentration. In some embodiments, the candidate inhibitors are added in amounts ranging from about 0.5 nM to about 10 μM. The incubation of candidate inhibitor with first and second cell groups can vary, typically ranging from about 30 minutes to about 60 hours. Exemplary inhibition assay conditions are provided in the Examples section below.

In some embodiments, in assays in which PI3K and/or mTOR mediated activity is being measured, the first and/or second cells can be stimulated with a growth factor. The selection of growth factor is mediated by the requirements of the cell line, for example, illustrative growth factors can include VEGF, IGF, insulin and heregulin.

In some embodiments, the inhibitory activity of the candidate compounds can be measured using a variety of cellular activities. When cancer cell lines are being used, the inhibition of PI3K mediated activity, e.g. AKT phosphorylation (both at residues S473 and T308), AKT activation, cellular proliferation, and apoptosis resistance in the cells can all be measured. In some embodiments, the amount of AKT phosphorylation in the first and second cell groups can be measured using a phopho-specific antibody (for example AKT1 (phospho S473, Cat. No. ab8932, AKT1 (phospho T308) Cat. No. ab66134) which are commercially available from AbCam, Cambridge, Mass. Other methods for measuring the inhibition of PI3K-α activity in the first and second cell groups are described in Donahue, A. C. et al., Measuring phosphorylated Akt and other phosphoinositide 3-kinase-regulated phosphoproteins in primary lymphocytes. Methods Enzymol. 2007(434):131-154 which is incorporated herein by reference in its entirety.

### Embodiment (Z)

In another embodiment, the invention provides a method for determining a treatment regimen for a cancer patient having a tumor comprising a PI3K-α, the method comprising:

determining the presence or absence of a mutation in amino acids 1047 and/or 545 of the PI3K-α;

wherein if the PI3K-α has a mutation at position 1047, the method comprises administering to the cancer patient a therapeutically effective amount of a PI3K-α selective inhibitor compound; or

wherein if the PI3K-α has a mutation at position 545, the method comprises administering to the cancer patient a therapeutically effective amount of a combination of a PI3K-α selective inhibitor and a PI3K-β selective inhibitor, a dual PI3K-α/mTOR selective inhibitor, or a combination of a PI3K-α selective inhibitor and a mTOR selective inhibitor.

In another embodiment, the invention provides a method for determining a treatment regimen for a cancer patient having a tumor comprising a PI3K-α, the method comprising:

determining the presence or absence of a mutation in amino acids 1047 and/or 545 of the PI3K-α;

wherein if the PI3K-α has a mutation at position 1047, the method comprises administering to the cancer patient a therapeutically effective amount of a PI3K-α selective inhibitor compound, a dual PI3K-α/mTOR selective inhibitor, a combination of a PI3K-α selective inhibitor and a mTOR selective inhibitor to the subject; or

wherein if the PI3K-α has a mutation at position 545, the method comprises administering to the cancer patient a therapeutically effective amount of a combination of a PI3K-α selective inhibitor and a PI3K-β selective inhibitor, a dual PI3K-α/mTOR selective inhibitor, or a combination of a PI3K-α selective inhibitor and a mTOR selective inhibitor.

The method of the invention can be used to identify cancer patient populations more likely to benefit from treatment with PI3Kα-selective inhibitors as well as patient populations less likely to benefit.

The invention can be used to further define genetic markers or gene expression signatures which identify PI3Kα inhibitor sensitive tumor subtypes by extended in vitro cell line profiling and in vivo pharmacodynamic and efficacy studies.

In some embodiments, a method for determining a treatment regimen for a cancer patient having the exemplified cancers herein can be readily performed on the basis of the differential activity of PI3K-α selective inhibitors in cancers having a PI3K-α mutated background described herein. In patients in which a tumor cell has been analyzed and assayed to determine whether the tumor harbors a PI3Kα mutation in the kinase domain, for example, a mutation resulting in H1047R, greater efficacy and treatment improvement can be achieved by tailoring a treatment comprising a PI3K-α selective inhibitor. For patients, who have a tumor which does not harbor a mutation in PI3Kα kinase domain, the treatment may require adopting a different treatment regimen, for example, by focusing on delivery of a combination of PI3K-α selective inhibitors and a PI3K-β selective inhibitor, a dual PI3K-α/mTOR selective inhibitor, or a combination of a PI3K-α selective inhibitor and a mTOR selective inhibitor. As indicated above, the PI3K-α selective inhibitors, mTOR selective inhibitors and dual PI3K-α/mTOR selective inhibitors are exemplified in Table 1 and in the detailed description herein.

In some embodiments, methods for determining a treatment regimen comprises determining the presence of a mutation in amino acids 1047 and/or 542 and/or 545 of the PI3K-α in the subject's tumor. In some embodiments, the mutation to the kinase domain can include a mutation to H1047 of SEQ ID NO:1 mutating to H1047R.

In some embodiments, the mutation to the helical domain can include a mutation to E542 of SEQ ID NO:1 mutating to E542K. In another embodiment, the mutation to the helical domain can include a mutation to E545 mutating to E545K. Exemplary mutations in the helical domain can include a mutation at position 542 and/or 545 of SEQ ID NO:1.

This step can be achieved in a variety of ways, using nucleic acid approaches, protein separation approaches or direct immunological approaches using mutation specific antibodies. In some embodiments, presence of a mutation in amino acids 1047 and/or 545 of the PI3K-α in the subject's tumor can be determined using any suitable method for the sequence analysis of amino acids. Examples of suitable techniques include, but are not limited to, western blot analysis, immunoprecipitation, radioimmunoassay (RIA) or enzyme-linked immunoabsorbent assay (ELISA).

In the present invention, reference to position within the amino acid sequence of PI3Kα is made referring to SEQ ID NO: 1. Reference to positions within the nucleotide sequence of the PI3Kα is made referring to SEQ ID NO:2. Specific amino acids in the wild type protein sequence are described using single letter amino acid designation followed by the position in the protein sequence, for example E545 indicates that position 545 is glutamic acid. To represent a substitution at a particular position, the substituted amino acid follows the position, for example E545K indicates that the glutamic acid at position 545 is replaced with a lysine.

Determining the presence or absence of mutations in the sequence of the PI3K-α peptide sequence is generally determined using in vitro methods wherein a tumor sample is used which has been removed from the body of a patient.

Determining the presence or absence of mutations in the amino acid sequence of PI3Kα or a portion thereof, can be done using any suitable method. For example the nucleotide sequence of PI3Kα or a portion thereof maybe determined and the amino acid sequence deduced from the nucleotide sequence or a PI3K-α protein can be interrogated directly.

The nucleotide sequence of the PI3K-α, or a portion thereof, may be determined using any method for the sequence analysis of nucleic acids. Methods for identification of sequence mutation in genes are well known in the art and the mutations in the PI3Kα can be identified by any suitable method. These methods include, but are not limited to, dynamic allele-specific hybridization; the use of molecular beacons; enzyme-based methods, using for example DNA ligase, DNA polymerase or nucleases; PCR based methods, whole genome sequencing; partial genome sequencing; exome sequencing; nucleic acid probe hybridization; and restriction enzyme digestion analysis.

Methods of Direct DNA sequencing are well known in the art, (see for example: Current Protocols in Molecular Biology, edited by Fred M. Ausubel, Roger Brent, Robert E. Kingston, David D. Moore, J. G. Seidman, John A. Smith, Kevin Struhl, and Molecular Cloning: A Laboratory Manual, Joe Sambrook, David W Russel, 3^{rd} edition, Cold Spring Harbor Laboratory Press).) These sequencing protocols include for example, the use of radioactively labeled nucleotides, and nucleotides labeled with a fluorescent dye.

For example, Barbi, S. et al., used the following protocol to sequence the helical domain (exon 9) and the kinase domain (exon 20) of PI3K-α. Normal and tumor DNA was extracted from paraffin-embedded tissue. and amplified using fluorescent dye-labeled primers. the following primer pairs. Primer sequences need to be chosen to uniquely select for a region of DNA, avoiding the possibility of mishybridization to a similar sequence nearby. A commonly used method is BLAST search whereby all the possible regions to which a primer may bind can be seen. Both the nucleotide sequence as well as the primer itself can be BLAST searched. The free NCBI tool Primer-BLAST integrates primer design tool and BLAST search into one application, so does commercial software product such as Beacon Designer, (Premier Biosoft International, Palo Alto Calif.). Mononucleotide repeats should be avoided, as loop formation can occur and contribute to mishybridization. In addition, computer programs are readily available to aid in design of suitable primers. In certain embodiments the nucleic acid probe is labeled for use in a Southern hybridization assay. The nucleic acid probe may be radioactively labeled, fluorescently labeled or is immunologically detectable, in particular is a digoxygenin-labeled (Roche Diagnostics GmbH, Mannheim).

In some embodiments, determining the presence of a helical domain mutation in exon 9 can be achieved using an exemplary forward primer and reverse primer, including, for example: GGGAAAAATATGACAAAGAAAGC (SEQ ID NO: 3) and CTGAGATCAGCCAAATTCAGTT (SEQ ID NO: 4) respectively and a sequencing primer can include TAGCTAGAGACAATGAATTAAGGGAAA (SEQ ID NO: 5), for determining a mutation in the kinase domain in exon 20, an exemplary set of primers can include forward and reverse primers CTCAATGATGCTTGGCTCTG (SEQ ID NO: 6) and TGGAATCCAGAGTGAGCTTTC (SEQ ID NO: 7) respectively and the sequencing primer can include TTGATGACATTGCATACATTCG (SEQ ID NO: 8). The amplification products were sequenced. (Barbi, S. et al. J. Experimental and Clinical Cancer Research 2010, 29:32) The sequences are then compared and differences between the wild type PI3K-α sequence and the sequence of the tumor PI3K-α. The assay could also be performed by only amplifying the tumor DNA and comparing the sequence with the sequence of SEQ ID NO:1.

In some embodiments, the present invention provides polynucleotide sequences comprising polynucleotide sequences in whole or in part from SEQ ID NO: 2 that are capable of hybridizing to the helical region, or the kinase domain of PI3K-α under conditions of high stringency. In some embodiments, the polynucleotides can include sequences complementary to nucleic acid sequences that encode in whole or in part PI3K-α or PI3K-α having specific mutations as described herein. The terms “complementary” and “complementarity” refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

In some embodiments, the present invention provides polynucleotide sequences comprising polynucleotide sequences in whole or in part from SEQ ID NO: 2 that are capable of hybridizing to the helical region, or the kinase domain oPI3K-α under conditions of high stringency. In some embodiments, the polynucleotides can include sequences complementary to nucleic acid sequences that encode in whole or in part PI3K-α or PI3K-α having specific mutations as described herein. The terms “complementary” and “complementarity” refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

“High stringency conditions” when used in reference to nucleic acid hybridization comprise conditions equivalent to binding or hybridization at 42 C.° in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 μg/mL denatured salmon sperm DNA followed by washing in a solution comprising 0.1×SSPE, 1.0% SDS at 42 C.° when a probe of about 500 nucleotides in length is employed.

The term “homology” when used in relation to nucleic acids refers to a degree of complementarity. There may be partial homology or complete homology (i.e., identity). “Sequence identity” refers to a measure of relatedness between two or more nucleic acids or proteins, and is given as a percentage with reference to the total comparison length. The identity calculation takes into account those nucleotide or amino acid residues that are identical and in the same relative positions in their respective larger sequences. Calculations of identity may be performed by algorithms contained within computer programs such as “GAP” (Genetics Computer Group, Madison, Wis.) and “ALIGN” (DNAStar, Madison, Wis.). A partially complementary sequence is one that at least partially inhibits (or competes with) a completely complementary sequence from hybridizing to a target nucleic acid is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a sequence which is completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

In preferred embodiments, hybridization conditions are based on the melting temperature (Tm) of the nucleic acid binding complex and confer a defined “stringency” The term “hybridization” refers to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids. A single molecule that contains pairing of complementary nucleic acids within its structure is said to be “self-hybridized.”

The term “Tm” refers to the “melting temperature” of a nucleic acid. The melting temperature is the temperature at which a population of double-stranded nucleic acid molecules becomes half dissociated into single strands. The equation for calculating the Tm of nucleic acids is well known in the art. As indicated by standard references, a simple estimate of the Tm value may be calculated by the equation: Tm=81.5+0.41(% G+C), when a nucleic acid is in aqueous solution at 1 M NaCl. The term “stringency” refers to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences.

In addition, sequence mutations in the PI3Kα can be determined using any sequence-specific nucleic acid detection method allowing detection of single-nucleotide variation, in particular any such method involving complementary base pairing. For example, to determine if the PI3K-α comprises a E545 mutation, the sequence of PI3K-α peptide or a portion thereof comprising nucleotides 1790, 1791 and 1792 of SEQ ID NO:2 (codon corresponding with position 545 in the amino acid sequence), is used in a polymerase chain reaction (PCR) where the oligonucleotide primers allow the amplification of PI3Kα only if the nucleotide at position 1790 is G. If no reaction product is formed then the amino acid at position 545 is mutated. In another example the oligonucleotide primers are designed to allow the amplification of the to allow amplification if the nucleotide at position 3297 is A (codon comprising nucleotides 3296, 3297 and 3298 corresponds with position 1047 of the amino acid sequence). If no reaction product is formed using those primers then the amino acid at position 545 is mutated. Methods for performing PCR are known in the art (see Current Protocols in Molecular Biology, edited by Fred M. Ausubel, Roger Brent, Robert E. Kingston, David D. Moore, J. G. Seidman, John A. Smith, Kevin Struhl. and; Molecular Cloning: A Laboratory Manual, Joe Sambrook, David W Russel, 3^{rd} edition, Cold Spring Harbor Laboratory Press).

Dynamic allele-specific hybridization (DASH) genotyping takes advantage of the differences in the melting temperature in DNA that results from the instability of mismatched base pairs. This technique is well suited to automation. In the first step, a DNA segment is amplified and attached to a bead through a PCR reaction with a biotinylated primer. In the second step, the amplified product is attached to a streptavidin column and washed with NaOH to remove the un-biotinylated strand. An sequence-specific oligonucleotide is then added in the presence of a molecule that fluoresces when bound to double-stranded DNA. The intensity is then measured as temperature is increased until the Tm can be determined. A single nucleotide change will result in a lower than expected Tm (Howell W., Jobs M., Gyllensten U., Brookes A. (1999) Dynamic allele-specific hybridization. A new method for scoring single nucleotide polymorphisms. Nat Biotechnol. 17(1):87-8). Because DASH genotyping is measuring a quantifiable change in Tm, it is capable of measuring all types of mutations, not just SNPs. Other benefits of DASH include its ability to work with label free probes and its simple design and performance conditions.

Molecular beacons can also be used to detect mutations in a DNA sequences Molecular beacons makes use of a specifically engineered single-stranded oligonucleotide probe. The oligonucleotide is designed such that there are complementary regions at each end and a probe sequence located in between. This design allows the probe to take on a hairpin, or stem-loop, structure in its natural, isolated state. Attached to one end of the probe is a fluorophore and to the other end a fluorescence quencher. Because of the stem-loop structure of the probe, the fluorophore is in close proximity to the quencher, thus preventing the molecule from emitting any fluorescence. The molecule is also engineered such that only the probe sequence is complementary to the to the genomic DNA that will be used in the assay (Abravaya K., Huff J., Marshall R., Merchant B., Mullen C., Schneider G., and Robinson J. (2003) Molecular beacons as diagnostic tools: technology and applications. Clin Chem Lab Med. 41:468-474). If the probe sequence of the molecular beacon encounters its target genomic DNA during the assay, it will anneal and hybridize. Because of the length of the probe sequence, the hairpin segment of the probe will denatured in favor of forming a longer, more stable probe-target hybrid. This conformational change permits the fluorophore and quencher to be free of their tight proximity due to the hairpin association, allowing the molecule to fluoresce. If on the other hand, the probe sequence encounters a target sequence with as little as one non-complementary nucleotide, the molecular beacon will preferentially stay in its natural hairpin state and no fluorescence will be observed, as the fluorophore remains quenched. The unique design of these molecular beacons allows for a simple diagnostic assay to identify SNPs at a given location. If a molecular beacon is designed to match a wild-type allele and another to match a mutant of the allele, the two can be used to identify the genotype of an individual. If only the first probe's fluorophore wavelength is detected during the assay then the individual is homozygous to the wild type. If only the second probe's wavelength is detected then the individual is homozygous to the mutant allele. Finally, if both wavelengths are detected, then both molecular beacons must be hybridizing to their complements and thus the individual must contain both alleles and be heterozygous.

Enzyme-based nucleic acid methods are also suitable and contemplated for determining mutations in the PI3K-α nucleotide sequence. For example, Restriction fragment length polymorphism (RFLP) (discussed in greater detail below) can be used to detect single nucleotide differences. SNP-RFLP makes use of the many different restriction endonucleases and their high affinity to unique and specific restriction sites. By performing a digestion on a genomic sample and determining fragment lengths through a gel assay it is possible to ascertain whether or not the enzymes cut the expected restriction sites. A failure to cut the genomic sample results in an identifiably larger than expected fragment implying that there is a mutation at the point of the restriction site which is rendering it protected from nuclease activity.

The term “functionally equivalent codon” is used herein to refer to codons that encode the same amino acid, such as the six codons for arginine.

In one embodiment of the invention, the method comprises at least one nucleic acid probe or oligonucleotide for determining the sequence of the codon that encodes amino acid 1047. In another embodiment the method comprises at least one nucleic acid probe or oligonucleotide for determining the sequence of the codon that encodes amino acid 545. The oligonucleotide is a PCR primer, preferably a set of PCR primers which allows amplification of a PI3Kα nucleic acid sequence fragment only if the codon which encodes amino acid 1047 encodes a histidine. In another method, the PCR primer or set of PCR primers allows the amplification of nucleic acid sequence fragment only if the codon which encodes amino acid 545 encodes a glutamic acid. Determination of suitable PCR primers is routine in the art, (Current Protocols in Molecular Biology, edited by Fred M. Ausubel, Roger Brent, Robert E. Kingston, David D. Moore, J. G. Seidman, John A. Smith, Kevin Struhl; Looseleaf: 0-471-650338-X; CD-ROM: 0-471-30661-4). In addition, computer programs are readily available to aid in design of suitable primers. In certain embodiments the nucleic acid probe is labeled for use in a Southern hybridization assay. The nucleic acid probe may be radioactively labeled, fluorescently labeled or is immunologically detectable, in particular is a digoxygenin-labeled (Roche Diagnostics GmbH, Mannheim).

U.S. Patent Publication 20010016323 discloses methods for detecting point mutations using a fluorescently labeled oligonucleotidemeric probe and fluorescence resonance energy transfer. A point mutation leading to a base mismatch between the probe and the target DNA strand causes the melting temperature of the complex to be lower than the melting temperature for the probe and the target if the probe and target were perfectly matched.

Other suitable methods for detecting single point mutations include those disclosed in, for example, U.S. Patent Publication 2002010665, which involves the use of oligonucleotide probes in array format. Such arrays can include one or more of SEQ ID NOs:3-8. U.S. Patent Publication 20020177157 discloses additional methods for detecting point mutations.

A polynucleotide carrying a point mutation leading to a mutation of PI3K-α kinase domain, for example, H1047R that is the subject of this invention can be identified using one or more of a number of available techniques. However, detection is not limited to the techniques described herein and the methods and compositions of the invention are not limited to these methods, which are provided for exemplary purposes only. Polynucleotide and oligonucleotide probes are also disclosed herein and are within the scope of the invention, and these probes are suitable for one or more of the techniques described below. These include allele-specific oligonucleotide hybridization (ASO), which, in one embodiment, is a diagnostic mutation detection method wherein hybridization with a pair of oligonucleotides corresponding to alleles of a known mutation is used to detect the mutation. Another suitable method is denaturing high performance liquid chromatography (DHPLC), which is a liquid chromatography method designed to identify mutations and polymorphisms based on detection of heteroduplex formation between mismatched nucleotides. Under specified conditions, heteroduplexes elute from the column earlier than homoduplexes because of reduced melting temperature. Analysis can then be performed on individual samples.

An amplified region of the DNA containing the mutation or the wild-type sequence can be analyzed by DHPLC. Use of DHPLC is described in U.S. Pat. Nos. 5,795,976 and 6,453,244, both of which are incorporated herein by reference. A suitable method is that provided by Transgenomic, Inc. (Omaha, Nebr.) using the Transgenomic WAVE® System.

For ASO, a region of genomic DNA or cDNA containing the PI3K-α mutation (H1047R and/or E545K) is amplified by PCR and transferred onto duplicating membranes. This can be performed by dot/slot blotting, spotting by hand, or digestion and Southern blotting. The membranes are prehybridized, then hybridized with a radiolabeled or deoxygenin (DIG) labeled oligonucleotide to either the mutant or wild-type sequences. For the DIG label, detection is performed using chemiluminescent or colorimetric methods. The membranes are then washed with increasing stringency until the ASO is washed from the non-specific sequence. Following autoradiographic exposure, the products are scored for the level of hybridization to each oligonucleotide. Optimally, controls are included for the normal and mutant sequence on each filter to confirm correct stringency, and a negative PCR control is used to check for contamination in the PCR.

The size of the ASO probe is not limited except by technical parameters of the art. Generally, too short a probe will not be unique to the location, and too long a probe may cause loss of sensitivity. The oligonucleotides are preferably 15-21 nucleotides in length, with the mismatch towards the center of the oligonucleotide.

The region of sample DNA on which ASO hybridization is performed to detect the mutation of this invention is preferably amplified by PCR using a forward primer, For exon 9 the forward primer and reverse primers were GGGAAAAATATGACAAAGAAAGC (SEQ ID NO: 3) and CTGAGATCAGCCAAATTCAGTT (SEQ ID NO: 4) respectively and the sequencing primer was TAGCTAGAGACAATGAATTAAGGGAAA (SEQ ID NO: 5), for exon 20 the forward and reverse primers were CTCAATGATGCTTGGCTCTG (SEQ ID NO: 6) and TGGAATCCAGAGTGAGCTTTC (SEQ ID NO: 7) respectively. In this case, amplification by PCR or a comparable method is not necessary but can optionally be performed.

Optionally, one or more than one of the amplified regions described above, (including the 306 nucleotide region generated using primers of SEQ ID NO:3-8, or shorter portions of either of these regions, can be analyzed by sequencing in order to detect the mutation. Sequencing can be performed as is routine in the art. The only limitation on choice of the region to be sequenced, in order to identify the presence of the mutation, is that the region selected for sequencing must include the nucleotide that is the subject of the mutation, The size of the region selected for sequencing is not limited except by technical parameters as is known in the art, and longer regions comprising part or all of the DNA or RNA between selected amplified regions using the primers SEQ ID NOs: 3 & 4 and 6 & 7 disclosed herein can be sequenced.

Variations of the methods disclosed above are also suitable for detecting the mutation. For example, in a variation of ASO, the ASO's are given homopolymer tails with terminal deoxyribonucleotidyl transferase, spotted onto nylon membrane, and covalently bound by UV irradiation. The target DNA is amplified with biotinylated primers and hybridized to the membrane containing the immobilized oligonucleotides, followed by detection. An example of this reverse dot blot technique is the INNO-LIPA kit from Innogenetics (Belgium).

With the identification and sequencing of the mutated gene and the gene product, i.e. SEQ ID NO:1 having a mutation at E545K and H1047R, probes and antibodies raised to the gene product can be used in a variety of hybridization and immunological assays to screen for and detect the presence of either a normal or mutated gene or gene product.

Expression of the mutated gene in heterologous cell systems can be used to demonstrate structure function relationships. Ligating the DNA sequence into a plasmid expression vector to transfect cells is a useful method to test the influence of the mutation on various cellular biochemical parameters. Plasmid expression vectors containing either the entire normal or mutant human or mouse sequence or portions thereof, can be used in in vitro mutagenesis experiments which will identify portions of the protein crucial for regulatory function.

The DNA sequence can be manipulated in studies to understand the expression of the gene and its product, and to achieve production of large quantities of the protein for functional analysis, for antibody production, and for patient therapy. Changes in the sequence may or may not alter the expression pattern in terms of relative quantities, tissue-specificity and functional properties.

A number of methods are available for analysis of variant (e.g., mutant or polymorphic) nucleic acid sequences. Assays for detections polymorphisms or mutations fall into several categories, including, but not limited to direct sequencing assays, fragment polymorphism assays, hybridization assays, and computer based data analysis. Protocols and commercially available kits or services for performing multiple variations of these assays are commercially available and known to those of skill in the art. In some embodiments, assays are performed in combination or in combined parts (e.g., different reagents or technologies from several assays are combined to yield one assay). The following illustrative assays may be used to screen and identify nucleic acid molecules containing the mutations of PI3K-α mutation of interest.

### Fragment Length Polymorphism Assays

In some embodiments of the present invention, variant sequences are detected using a fragment length polymorphism assay. In a fragment length polymorphism assay, a unique DNA banding pattern based on cleaving the DNA at a series of positions is generated using an enzyme (e.g., a restriction enzyme or a CLEAVASE I [Third Wave Technologies, Madison, Wis.] enzyme). DNA fragments from a sample containing a SNP or a mutation will have a different banding pattern than wild type.

### PCR Assays

In some embodiments of the present invention, variant sequences are detected using a PCR-based assay. In some embodiments, the PCR assay comprises the use of oligonucleotide nucleic acid primers that hybridize only to the variant or wild type allele of EFHD1 (e.g., to the region of mutation or multiple mutations). Both sets of primers are used to amplify a sample of DNA. If only the mutant primers result in a PCR product, then the subject's tumor or cancer expresses a somatic mutation in an PI3K-α mutation allele. PCR amplification conditions are tailored to the specific oligonucleotide primers or oligonucleotide probes used, the quality and type of DNA or RNA being screened, and other well known variables that can be controlled using appropriate reagents and/or PCR cycling conditions known to those of ordinary skill in the art.

### RFLP Assays

In some embodiments of the present invention, variant sequences are detected using a restriction fragment length polymorphism assay (RFLP). The region of interest is first isolated using PCR. The PCR products are then cleaved with restriction enzymes known to give a unique length fragment for a given polymorphism. The restriction-enzyme digested PCR products are separated by agarose gel electrophoresis and visualized by ethidium bromide staining. The length of the fragments is compared to molecular weight markers and fragments generated from wild-type and mutant controls.

### Direct Sequencing Assays

In some embodiments of the present invention, variant sequences are detected using a direct sequencing technique. In these assays, DNA samples are first isolated from a subject using any suitable method. In some embodiments, the region of interest is cloned into a suitable vector and amplified by growth in a host cell (e.g., a bacteria). In other embodiments, DNA in the region of interest is amplified using PCR.

Following amplification, DNA in the region of interest (e.g., the region containing the SNP or mutation of interest) is sequenced using any suitable method, including but not limited to manual sequencing using radioactive marker nucleotides, or automated sequencing. The results of the sequencing are displayed using any suitable method. The sequence is examined and the presence or absence of a given SNP or mutation is determined.

### CFLP Assays

In other embodiments, variant sequences are detected using a CLEAVASE fragment length polymorphism assay (CFLP; Third Wave Technologies, Madison, Wis.; See e.g., U.S. Pat. Nos. 5,843,654; 5,843,669; 5,719,208; and 5,888,780; each of which is herein incorporated by reference). This assay is based on the observation that when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme, is a structure-specific, thermostable nuclease that recognizes and cleaves the junctions between these single-stranded and double-stranded regions. The region of interest is first isolated, for example, using PCR. Then, DNA strands are separated by heating. Next, the reactions are cooled to allow intra-strand secondary structure to form. The PCR products are then treated with the CLEAVASE I enzyme to generate a series of fragments that are unique to a given SNP or mutation. The CLEAVASE enzyme treated PCR products are separated and detected (e.g., by agarose gel electrophoresis) and visualized (e.g., by ethidium bromide staining). The length of the fragments is compared to molecular weight markers and fragments generated from wild-type and mutant controls.

### Hybridization Assays

In some embodiments of the present invention, variant sequences are detected by hybridization analysis in a hybridization assay. In a hybridization assay, the presence or absence of a given mutation is determined based on the ability of the DNA from the sample to hybridize to a complementary DNA molecule (e.g., a oligonucleotide probe or probes as illustrated herein). A variety of hybridization assays using a variety of technologies for hybridization and detection are available. Relevant and useful hybridization assays for practicing the methods of the present invention are provided below.

### Direct Detection of Hybridization

In some embodiments, hybridization of a probe to the sequence of interest (e.g., a SNP or mutation) is detected directly by visualizing a bound probe (e.g., a Northern or Southern assay; See e.g., Ausabel et al. (eds.) (1991) Current Protocols in Molecular Biology, John Wiley & Sons, NY). In a these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., on an agarose gel) and transferred to a membrane. A labeled (e.g., by incorporating a radionucleotide) probe or probes specific for the SNP or mutation being detected is allowed to contact the membrane under a condition or low, medium, or high stringency conditions. The unbound probe is removed and the presence of binding is detected by visualizing the labeled probe.

### Detection of Hybridization Using “DNA Chip” Assays

In some embodiments of the present invention, variant sequences are detected using a DNA chip hybridization assay. In this assay, a series of oligonucleotide probes are affixed to a solid support. The oligonucleotide probes are designed to be unique to a given SNP or mutation. The DNA sample of interest is contacted with the DNA “chip” and hybridization is detected.

In some embodiments, an illustrative and commercially available DNA chip assay can include a GENECHIP® (commercially available from Affymetrix, Santa Clara, Calif., USA); See e.g., U.S. Pat. Nos. 6,045,996; 5,925,525; and 5,858,659; each of which is herein incorporated by reference) assay. The GENECHIP® technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

### Enzymatic Detection of Hybridization

In some embodiments of the present invention, hybridization can be detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with fluorescein that is quenched by an internal dye. Upon cleavage, the de-quenched fluorescein labeled product may be detected using a standard fluorescence plate reader. The INVADER assay detects specific mutations in unamplified genomic DNA. The isolated DNA sample is contacted with the first probe specific either for a mutation of the present invention or wild type PI3K-α sequence and allowed to hybridize. Then a secondary probe, specific to the first probe, and containing the fluorescein label, is hybridized and the enzyme is added. Binding is detected by using a fluorescent plate reader and comparing the signal of the test sample to known positive and negative controls.

In some embodiments, hybridization of a bound probe is detected using a TaqMan assay (PE Biosystems, Foster City, Calif.; See e.g., U.S. Pat. Nos. 5,962,233 and 5,538,848, each of which is herein incorporated by reference). The assay is performed during a PCR reaction. The TaqMan assay exploits the 5′-3′ exonuclease activity of the AMPLITAQ GOLD DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorometer.

In accordance with the present invention, diagnostic kits are also provided which will include the reagents necessary for the above-described diagnostic screens. For example, kits may be provided which include oligonucleotide probes or PCR primers are present for the detection and/or amplification of mutant EFHD1, and comparable wild-type EFHD1-related nucleotide sequences. Again, such probes may be labeled for easier detection of specific hybridization. As appropriate to the various diagnostic embodiments described above, the oligonucleotide probes in such kits may be immobilized to substrates and appropriate controls may be provided. Examples of such oligonucleotide probes include oligonucleotides comprising or consisting of at least one of SEQ ID NOs:3&4 and 6&7.

Determining the presence or absence of mutations in the amino acid sequence of PI3Kα can be determined using any method for the sequence analysis of amino acids. Non-limiting examples include: western blot analysis or ELISA assays, or direct protein sequencing of the PI3Kα in the subject's tumor. In some embodiments, particularly useful antibodies have selectivity for wild type PI3K-α versus the mutant PI3Kα, for example, an antibody useful in the assay would bind to wild type PI3K-α, or a portion wild type PI3Kα, but not to a PI3Kα having a mutation at the amino acid of interest. Particularly useful antibodies could include antibodies which bind the wild type PI3Kα which has histidine at position 1047 but does not bind a mutant PI3Kα which has an amino acid other than histidine, such as arginine, in other words the antibody specifically bind to an epitope comprising histidine at position 1047. Likewise, particularly useful are antibodies which bind the wild type PI3Kα which has glutamic acid at position 545 but does not bind a mutant PI3Kα which has an amino acid other than glutamic acid at position 545, such as lysine at that position.

Another embodiment of the invention provides a method comprising the use of at least one antibody which binds selectively to the wild type PI3Kα protein as compared with binding to a mutated form of PI3Kα. Alternately the antibody binds selectively to a mutated form of PI3Kα as compared with binding to the wild type PI3Kα protein and can differentiate between wild-type PI3Kα and PI3Kα-H1047R or between wild-type PI3Kα and PI3Kα-E545K. Methods for isolating suitable amounts of target protein from a complex mixture in relatively small amounts (less than 1 mg) are commonly known by those skilled in the art. In one illustrative embodiment, a tumor cell or plurality of tumor cells from a subject's tumor or cancer are lysed using commonly available lysing reagents in the presence of protease inhibitors. The lysate is cleared and the supernatant is either electrophoresed and subjected to a Western Blot using mutation specific antibodies, or alternatively, the mutated PI3Kα-H1047R or PI3Kα-E545K are selectively immunoprecipitated and further dissociated from the capture antibody and subjected to Western Blotting or protein sequenced directly.

“Antibody” includes, any immunoglobulin molecule that recognizes and specifically binds to a target, such as a protein, polypeptide, peptide, carbohydrate, polynucleotide, lipid, etc., through at least one antigen recognition site within the variable region of the immunoglobulin molecule. As used herein, the term is used in the broadest sense and encompasses intact polyclonal antibodies, intact monoclonal antibodies, antibody fragments (such as Fab, Fab′, F(ab′)₂, and Fv fragments), single chain Fv (scFv) mutants, multispecific antibodies such as bispecific antibodies generated from at least two intact antibodies, fusion proteins comprising an antibody portion, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA 1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, and the like.

“Antibody fragment” can refer to a portion of an intact antibody. Examples of antibody fragments include, but are not limited to, linear antibodies; single-chain antibody molecules; Fc or Fc′ peptides, Fab and Fab fragments, and multispecific antibodies formed from antibody fragments.

“Chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g. mouse, rat, rabbit, etc) with the desired specificity, affinity, and capability while the constant regions are homologous to the sequences in antibodies derived from another (usually human) to avoid eliciting an immune response in that species.

“Humanized” forms of non-human (e.g., rabbit) antibodies include chimeric antibodies that contain minimal sequence, or no sequence, derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues that are not found in the recipient antibody or in the donor antibody. Most often, the humanized antibody can comprise substantially all of at least one, and typically two, variable domains, in which all or substantially all of the hypervariable loops correspond to those of a nonhuman immunoglobulin and all or substantially all of the FR residues are those of a human immunoglobulin sequence. The humanized antibody can also comprise at least a portion of an immunoglobulin constant region (Fc), typically that of a human immunoglobulin. Methods used to generate humanized antibodies are well known in the field of immunology and molecular biology.

“Hybrid antibodies” can include immunoglobulin molecules in which pairs of heavy and light chains from antibodies with different antigenic determinant regions are assembled together so that two different epitopes or two different antigens can be recognized and bound by the resulting tetramer.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3-5, and more usually, at least 5 or 8-10 amino acids in a unique spatial conformation.

“Specifically binds” to or shows “specific binding” towards an epitope means that the antibody reacts or associates more frequently, and/or more rapidly, and/or greater duration, and/or with greater affinity with the epitope than with alternative substances.

### Preparation of Antibodies

### Polyclonal Antibodies

Polyclonal antibodies are preferably raised in animals by multiple subcutaneous (sc) or intraperitoneal (ip) injections of the relevant antigen and an adjuvant. Alternatively, antigen may be injected directly into the animal's lymph node (see Kilpatrick et al., Hybridoma, 16:381-389, 1997). An improved antibody response may be obtained by conjugating the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride or other agents known in the art.

Animals are immunized against the antigen, immunogenic conjugates or derivatives by combining, e.g., 100 μg of the protein or conjugate (for mice) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. At 7-14 days post-booster injection, the animals are bled and the serum is assayed for antibody titer. Animals are boosted until the titer plateaus. Preferably, the animal is boosted with the conjugate of the same antigen, but conjugated through a different cross-linking reagent. Conjugates also can be made in recombinant cell culture as protein fusions. Also, aggregating agents such as alum are suitably used to enhance the immune response.

### Monoclonal Antibodies

Monoclonal antibodies can be made using the hybridoma method first described by Kohler et al., Nature, 256:495 (1975), or by recombinant DNA methods. In the hybridoma method, a mouse or other appropriate host animal, such as rats, hamster or macaque monkey, is immunized to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells and are sensitive to a medium. Human myeloma and mouse-human heteromyeloma cell lines also have been described for the production of human monoclonal antibodies (Kozbor, J. Immunol., 133: 3001 (1984); Brodeur et al., Monoclonal Antibody Production Techniques and Applications, pp. 51-63 (Marcel Dekker, Inc., New York, 1987)). Exemplary murine myeloma lines include those derived from MOP-21 and M. C.-11 mouse tumors available from the Salk Institute Cell Distribution Center, San Diego, Calif. USA, and SP-2 or X63-Ag8-653 cells available from the American Type Culture Collection, Rockville, Md. USA. Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). The binding affinity of the monoclonal antibody can be determined, for example, by BIAcore or Scatchard analysis (Munson et al., Anal. Biochem., 107:220 (1980)).

After hybridoma cells are identified that produce antibodies of the desired specificity, affinity, and/or activity, the clones can be subcloned by limiting dilution procedures and grown by standard methods (Goding, Monoclonal Antibodies: Principles and Practice, pp. 59-103 (Academic Press, 1986)). Suitable culture media for this purpose include, for example, D-MEMO or RPMI 1640 medium. In addition, the hybridoma cells can be grown in vivo as ascites tumors in an animal. The monoclonal antibodies secreted by the subclones are suitably separated from the culture medium, ascites fluid, or serum by conventional immunoglobulin purification procedures such as protein A-Sepharose, hydroxylapatite chromatography, gel electrophoresis, dialysis, or affinity chromatography.

### Recombinant Production of Antibodies

The amino acid sequence of an immunoglobulin of interest can be determined by direct protein sequencing, and suitable encoding nucleotide sequences can be designed according to a universal codon table.

Alternatively, DNA encoding the monoclonal antibodies can be isolated and sequenced from the hybridoma cells using conventional procedures (e.g., by using oligonucleotide probes that are capable of binding specifically to genes encoding the heavy and light chains of the monoclonal antibodies). Sequence determination will generally require isolation of at least a portion of the gene or cDNA of interest. Usually this requires cloning the DNA or mRNA encoding the monoclonal antibodies. Cloning is carried out using standard techniques (see, e.g., Sambrook et al. (1989) Molecular Cloning: A Laboratory Guide, Vols 1-3, Cold Spring Harbor Press, which is incorporated herein by reference). For example, a cDNA library can be constructed by reverse transcription of polyA+ mRNA, preferably membrane-associated mRNA, and the library screened using probes specific for human immunoglobulin polypeptide gene sequences. In a preferred embodiment, the polymerase chain reaction (PCR) is used to amplify cDNAs (or portions of full-length cDNAs) encoding an immunoglobulin gene segment of interest (e.g., a light chain variable segment). The amplified sequences can be cloned readily into any suitable vector, e.g., expression vectors, minigene vectors, or phage display vectors. It will be appreciated that the particular method of cloning used is not critical, so long as it is possible to determine the sequence of some portion of the immunoglobulin polypeptide of interest.

One source for RNA used for cloning and sequencing is a hybridoma produced by obtaining a B cell from the transgenic mouse and fusing the B cell to an immortal cell. An advantage of using hybridomas is that they can be easily screened, and a hybridoma that produces a human monoclonal antibody of interest selected. Alternatively, RNA can be isolated from B cells (or whole spleen) of the immunized animal. When sources other than hybridomas are used, it may be desirable to screen for sequences encoding immunoglobulins or immunoglobulin polypeptides with specific binding characteristics. One method for such screening is the use of phage display technology. Phage display is described in e.g., Dower et al., WO 91/17271, McCafferty et al., WO 92/01047, and Caton and Koprowski, Proc. Natl. Acad. Sci. USA, 87:6450-6454 (1990), each of which is incorporated herein by reference. In one embodiment using phage display technology, cDNA from an immunized transgenic mouse (e.g., total spleen cDNA) is isolated, PCR is used to amplify cDNA sequences that encode a portion of an immunoglobulin polypeptide, e.g., CDR regions, and the amplified sequences are inserted into a phage vector. cDNAs encoding peptides of interest, e.g., variable region peptides with desired binding characteristics, are identified by standard techniques such as panning. The sequence of the amplified or cloned nucleic acid is then determined. Typically the sequence encoding an entire variable region of the immunoglobulin polypeptide is determined, however, sometimes only a portion of a variable region need be sequenced, for example, the CDR-encoding portion. Typically the sequenced portion will be at least 30 bases in length, and more often bases coding for at least about one-third or at least about one-half of the length of the variable region will be sequenced. Sequencing can be carried out on clones isolated from a cDNA library or, when PCR is used, after subcloning the amplified sequence or by direct PCR sequencing of the amplified segment. Sequencing is carried out using standard techniques (see, e.g., Sambrook et al. (1989) Molecular Cloning: A Laboratory Guide, Vols 1-3, Cold Spring Harbor Press, and Sanger, F. et al. (1977) Proc. Natl. Acad. Sci. USA 74: 5463-5467, which is incorporated herein by reference). By comparing the sequence of the cloned nucleic acid with published sequences of human immunoglobulin genes and cDNAs, an artisan can determine readily, depending on the region sequenced, (i) the germline segment usage of the hybridoma immunoglobulin polypeptide (including the isotype of the heavy chain) and (ii) the sequence of the heavy and light chain variable regions, including sequences resulting from N-region addition and the process of somatic mutation. One source of immunoglobulin gene sequence information is the National Center for Biotechnology Information, National Library of Medicine, National Institutes of Health, Bethesda, Md.

Once isolated, the DNA may be operably linked to expression control sequences or placed into expression vectors, which are then transfected into host cells such as E. coli cells, simian COS cells, Chinese hamster ovary (CHO) cells, or myeloma cells that do not otherwise produce immunoglobulin protein, to direct the synthesis of monoclonal antibodies in the recombinant host cells.

Expression control sequences denote DNA sequences necessary for the expression of an operably linked coding sequence in a particular host organism. The control sequences that are suitable for prokaryotes, for example, include a promoter, optionally an operator sequence, and a ribosome-binding site. Eukaryotic cells are known to utilize promoters, polyadenylation signals, and enhancers.

Nucleic acid is operably linked when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome-binding site is operably linked to a coding sequence if it is positioned so as to facilitate translation. Generally, operably linked means that the DNA sequences being linked are contiguous, and, in the case of a secretory leader, contiguous and in reading phase. However, enhancers do not have to be contiguous. Linking can be accomplished by ligation at convenient restriction sites. If such sites do not exist, synthetic oligonucleotide adaptors or linkers can be used in accordance with conventional practice.

Cell, cell line, and cell culture are often used interchangeably and all such designations include progeny. Transformants and transformed cells include the primary subject cell and cultures derived therefrom without regard for the number of transfers. It also is understood that all progeny may not be precisely identical in DNA content, due to deliberate or inadvertent mutations. Mutant progeny that have the same function or biological activity as screened for in the originally transformed cell are included.

Isolated nucleic acids also are provided that encode specific antibodies, optionally operably linked to control sequences recognized by a host cell, vectors and host cells comprising the nucleic acids, and recombinant techniques for the production of the antibodies, which may comprise culturing the host cell so that the nucleic acid is expressed and, optionally, recovering the antibody from the host cell culture or culture medium.

A variety of vectors are known in the art. Vector components can include one or more of the following: a signal sequence (that, for example, can direct secretion of the antibody), an origin of replication, one or more selective marker genes (that, for example, can confer antibiotic or other drug resistance, complement auxotrophic deficiencies, or supply critical nutrients not available in the media), an enhancer element, a promoter, and a transcription termination sequence, all of which are well known in the art.

Suitable host cells include prokaryote, yeast, or higher eukaryote cells. Suitable prokaryotes include eubacteria, such as Gram-negative or Gram-positive organisms, for example, Enterohacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas, and Streptomyces. In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available, such as Pichia, e.g. P. pastoris, Schizosaccharomyces pombe; Kluyveromyces, Yarrowia; Candida; Trichoderma reesia; Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-I variant of Autographa californica NPV and the Bm-5 strain of Bombyx mori NPV.

However, interest has been greatest in vertebrate cells, and propagation of vertebrate cells in culture (tissue culture) has become routine. Examples of useful mammalian host cell-lines are Chinese hamster ovary cells, including CHOKI cells (ATCC CCL61) and Chinese hamster ovary cells/−DHFR (DXB-11, DG-44; Urlaub et al, Proc. Natl. Acad. Sci. USA 77: 4216 (1980)); monkey kidney CV1 line transformed by SV40 (COS-7, ATCC CRL 1651); human embryonic kidney line (293 or 293 cells subcloned for growth in suspension culture, [Graham et al., J. Gen Virol. 36: 59 (1977)]; baby hamster kidney cells (BHK, ATCC CCL 10); mouse Sertoli cells (TM4, Mather, Biol. Reprod. 23: 243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (WI38, ATCC CCL 75); human hepatoma cells (Hep G2, HB 8065); mouse mammary tumor (MMT 060562, ATCC CCL51); TR1 cells (Mather et al., Annals N.Y. Acad. Sci. 383: 44-68 (1982)); MRC 5 cells and FS4 cells.

The host cells can be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44 (1979), Barnes et al., Anal. Biochem. 102: 255 (1980), U.S. Pat. No. 4,767,704; 4,657,866; 4,927,762; 4,560,655; or 5,122,469; WO90103430; WO 87/00195; or U.S. Pat. Re. No. 30,985 can be used as culture media for the host cells. Any of these media can be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements also can be included at appropriate concentrations that would be known to those skilled in the art. The culture conditions, such as temperature, pH, and the like, are those previously used with the host cell selected for expression, and will be apparent to the artisan.

The antibody composition can be purified using, for example, hydroxylapatite chromatography, cation or anion exchange chromatography, or preferably affinity chromatography, using the antigen of interest or protein A or protein G as an affinity ligand. Protein A can be used to purify antibodies that are based on human .gamma.1, .gamma.2, or .gamma.4 heavy chains (Lindmark et al., J. Immunol. Meth. 62: 1-13 (1983)). Protein G is recommended for all mouse isotypes and for human .gamma.3 (Guss et al., 20 EMBO J. 5: 15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, 25 NJ.) is useful for purification. Other techniques for protein purification such as ethanol precipitation, Reverse Phase HPLC, chromatofocusing, SDS-PAGE, and ammonium sulfate precipitation are also possible depending on the specific binding agent or antibody to be recovered.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3-5, and more usually, at least 5 or 8-10 amino acids in a unique spatial conformation.

“Specifically binds” to or shows “specific binding” towards an epitope means that the antibody reacts or associates more frequently, and/or more rapidly, and/or greater duration, and/or with greater affinity with the epitope than with alternative substances.

In some embodiments, once the subject's tumor has been analyzed to determine whether the tumor harbors a wild type PI3K-α versus a mutant PI3K-α, for example, PI3K-α E545K or PI3K-α H1047R, using any one or more of the assays and methods described above, a treatment regimen can be prepared for the subject. If the subject's tumor harbors a PI3K-α having a mutation at position 1047, (for example, H1047R), the treatment regimen comprises administering to the subject a therapeutically effective amount of a PI3K-α selective inhibitor compound, or a dual PI3K-α/mTOR selective inhibitor, or a combination of a PI3K-α selective inhibitor or a mTOR selective inhibitor. If the subject's tumor harbors a PI3K-α having a mutation at position 545, (for example, E545K), the treatment regimen comprises administering to the subject a therapeutically effective amount of a combination of a PI3K-α selective inhibitor and a PI3K-β selective inhibitor, a dual PI3K-α/mTOR selective inhibitor, or a combination of a PI3K-α selective inhibitor and a mTOR selective inhibitor.

### Embodiment (AZ)

In another embodiment, the present invention provides kits comprising materials useful for carrying out the methods of the invention. The diagnostic/screening procedures described herein may be performed by diagnostic laboratories, experimental laboratories, or practitioners. The invention provides kits which can be used in these different settings.

Basic materials and reagents required for identifying a PI3K-α mutation in a subject's tumor or cancer according to methods of the present invention may be assembled together in a kit. In certain embodiments, the kit comprises at least one PI3K-α amino acid sequence determining reagent that specifically detects a mutation in a nucleic acid or protein obtained from a subject's tumor disclosed herein, and instructions for using the kit according to one or more methods of the invention. Each kit necessarily comprises reagents which render the procedure specific. Thus, for detecting mRNA harboring the PI3K-α H1047R or E545K mutation, the reagent will comprise a nucleic acid probe complementary to mRNA, such as, for example, a cDNA or an oligonucleotide. The nucleic acid probe may or may not be immobilized on a substrate surface (e.g., a microarray). For detecting a polypeptide product encoded by at least one PI3K-α mutation gene, the reagent will comprise an antibody that specifically binds to the mutated PI3K-α or a wild-type PI3K-α.

Depending on the procedure, the kit may further comprise one or more of: extraction buffer and/or reagents, amplification buffer and/or reagents, hybridization buffer and/or reagents, immunodetection buffer and/or reagents, labeling buffer and/or reagents, and detection means. Protocols for using these buffers and reagents for performing different steps of the procedure may also be included in the kit.

Reagents may be supplied in a solid (e.g., lyophilized) or liquid form. Kits of the present invention may optionally comprise one or more receptacles for mixing samples and/or reagents (e.g., vial, ampoule, test tube, ELISA plate, culture plate, flask or bottle) for each individual buffer and/or reagent. Each component will generally be suitable as aliquoted in its respective container or provided in a concentrated form. Other containers suitable for conducting certain steps for the disclosed methods may also be provided. The individual containers of the kit are preferably maintained in close confinement for commercial sale.

In certain embodiments, the kits of the present invention further comprise control samples. For example, a kit may include samples of total mRNA derived from tissue of various physiological states, such as, for example, wild-type PI3K-α, PI3K-α H1047R mRNA or PI3K-α E545K mRNA to be used as controls. In other embodiments, the inventive kits comprise at least one prostate disease expression profile map as described herein for use as comparison template. Preferably, the expression profile map is digital information stored in a computer-readable medium.

Instructions for using the kit according to one or more methods of the invention may comprise instructions for processing the prostate tissue sample and/or performing the test, instructions for interpreting the results as well as a notice in the form prescribed by a governmental agency (e.g., FDA) regulating the manufacture, use or sale of pharmaceuticals or biological products.

### Representative Compounds

Representative compounds of Formula I are depicted below. The examples are merely illustrative and do not limit the scope of the invention in any way. Compounds of the invention are named according to systematic application of the nomenclature rules agreed upon by the International Union of Pure and Applied Chemistry (IUPAC), International Union of Biochemistry and Molecular Biology (IUBMB), and the Chemical Abstracts Service (CAS). Specifically, names in Table 1 were generated using ACD/Labs naming software 8.00 release, product version 8.08 or later.

TABLE 1

| Cmpd | | |
|---|---|---|
| No. | Structure | Name |
| 1 | 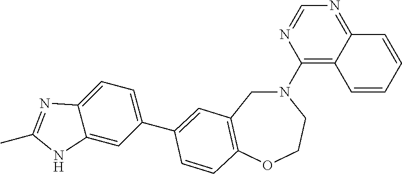 [SMILES: Cc1nc2ccc(-c3ccc4c(c3)CN(c3ncnc5ccccc35)CCO4)cc2[nH]1] | 7-(2-methyl-1H-benzimidazol-6- yl)-4-quinazolin-4-yl-2,3,4,5- tetrahydro-1,4-benzoxazepine |
| 2 | 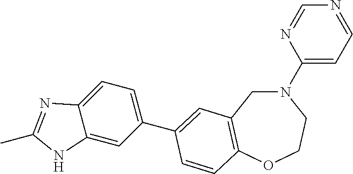 [SMILES: Cc1nc2ccc(-c3ccc4c(c3)CN(c3ccncn3)CCO4)cc2[nH]1] | 7-(2-methyl-1H-benzimidazol-6- yl)-4-pyrimidin-4-yl-2,3,4,5- tetrahydro-1,4-benzoxazepine |
| 3 | 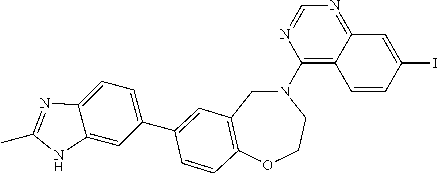 [SMILES: Cc1nc2ccc(-c3ccc4c(c3)CN(c3ncnc5cc(I)ccc35)CCO4)cc2[nH]1] | 4-(7-iodoquinazolin-4-yl)-7-(2- methyl-1H-benzimidazol-6-yl)- 2,3,4,5-tetrahydro-1,4- benzoxazepine |
| 4 | 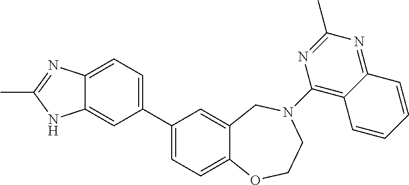 [SMILES: Cc1nc(N2CCOc3ccc(-c4ccc5nc(C)[nH]c5c4)cc3C2)c2ccccc2n1] | 7-(2- methyl-1H-benzimidazol-6- yl)-4-(2-methylquinazolin-4-yl)- 2,3,4,5-tetrahydro-1,4- benzoxazepine |
| 5 | 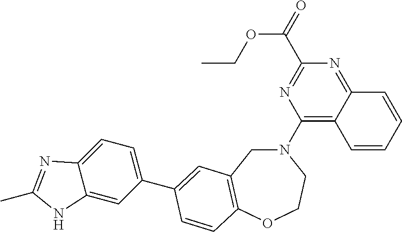 [SMILES: CCOC(=O)c1nc(N2CCOc3ccc(-c4ccc5nc(C)[nH]c5c4)cc3C2)c2ccccc2n1] | ethyl 4-[7-(2-methyl-1H- benzimidazol-6-yl)-2,3-dihydro- 1,4-benzoxazepin-4(5H)- yl]quinazoline-2-carboxylate |
| 6 | 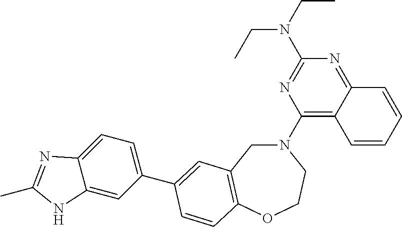 [SMILES: CCN(CC)c1nc(N2CCOc3ccc(-c4ccc5nc(C)[nH]c5c4)cc3C2)c2ccccc2n1] | N,N-diethyl-4-[7-(2-methyl-1H- benzimidazol-6-yl)-2,3-dihydro- 1,4-benzoxazepin-4(5H)- yl]quinazolin-2-amine |
| 7 | 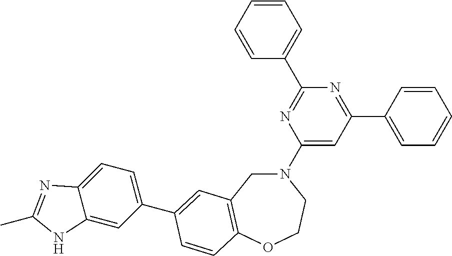 [SMILES: Cc1nc2ccc(-c3ccc4c(c3)CN(c3cc(-c5ccccc5)nc(-c5ccccc5)n3)CCO4)cc2[nH]1] | 4-(2,6-diphenylpyrimidin-4-yl)- 7-(2-methyl-1H-benzimidazol-6- yl)-2,3,4,5-tetrahydro-1,4- benzoxazepine |
| 8 | 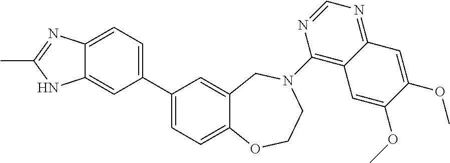 | 4-[6,7- bis(methyloxy)quinazolin-4-yl]- 7-(2-methyl-1H-benzimidazol-6- yl)-2,3,4,5-tetrahydro-1,4- benzoxazepine |
| 9 | 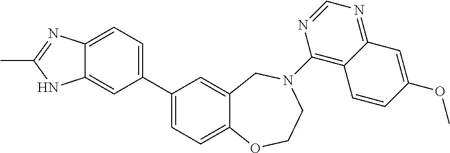 | 7-(2-methyl-1H-benzimidazol-6- yl)-4-[7-(methyloxy)quinazolin- 4-yl]-2,3,4,5-tetrahydro-1,4- benzoxazepine |
| 10 | 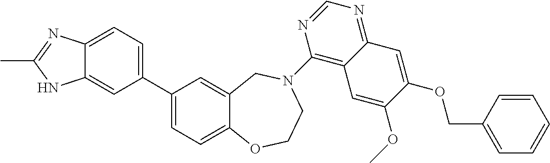 [SMILES: COc1cc2c(N3CCOc4ccc(-c5ccc6nc(C)[nH]c6c5)cc4C3)ncnc2cc1OCc1ccccc1] | 7-(2-methyl-1H-benzimidazol-6- yl)-4-{6-(methyloxy)-7- [(phenylmethyl)oxy]quinazolin- 4-yl}-2,3,4,5-tetrahydro-1,4- benzoxazepine |
| 11 | 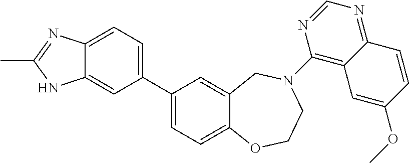 [SMILES: COc1ccc2ncnc(N3CCOc4ccc(-c5ccc6nc(C)[nH]c6c5)cc4C3)c2c1] | 7-(2-methyl-1H-benzimidazol-6- yl)-4-[6-(methyloxy)quinazolin- 4-yl]-2,3,4,5-tetrahydro-1,4- benzoxazepine |
| 12 | 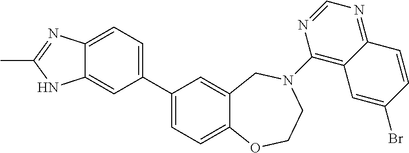 [SMILES: Cc1nc2ccc(-c3ccc4c(c3)CN(c3ncnc5ccc(Br)cc35)CCO4)cc2[nH]1] | 4-(6-bromoquinazolin-4-yl)-7- (2-methyl-1H-benzimidazol-6- yl)-2,3,4,5-tetrahydro-1,4- benzoxazepine |
| 13 | 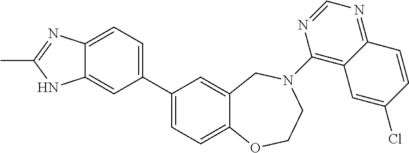 [SMILES: Cc1nc2ccc(-c3ccc4c(c3)CN(c3ncnc5ccc(Cl)cc35)CCO4)cc2[nH]1] | 4-(6-chloroquinazolin-4-yl)-7- (2-methyl-1H-benzimidazol-6- yl)-2,3,4,5-tetrahydro-1,4- benzoxazepine |
| 14 | 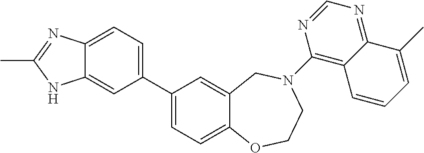 | 7-(2-methyl-1H-benzimidazol-6- yl)-4-(8-methylquinazolin-4-yl)- 2,3,4,5-tetrahydro-1,4- benzoxazepine |
| 15 | 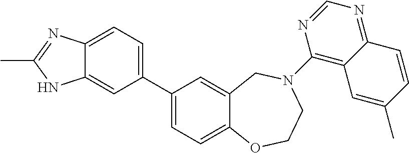 [SMILES: Cc1ccc2ncnc(N3CCOc4ccc(-c5ccc6nc(C)[nH]c6c5)cc4C3)c2c1] | 7-(2-methyl-1H-benzimidazol-6- yl)-4-(6-methylquinazolin-4-yl)- 2,3,4,5-tetrahydro-1,4- benzoxazepine |
| 16 | 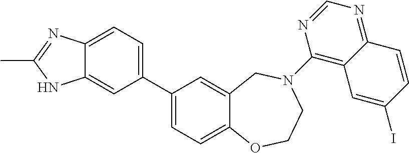 [SMILES: Cc1nc2ccc(-c3ccc4c(c3)CN(c3ncnc5ccc(I)cc35)CCO4)cc2[nH]1] | 4-(6-iodoquinazolin-4-yl)-7-(2- methyl-1H-benzimidazol-6-yl)- 2,3,4,5-tetrahydro-1,4- benzoxazepine |
| 17 | 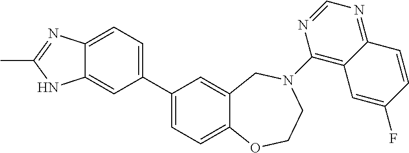 [SMILES: Cc1nc2ccc(-c3ccc4c(c3)CN(c3ncnc5ccc(F)cc35)CCO4)cc2[nH]1] | 4-(6-fluoroquinazolin-4-yl)-7-(2- methyl-1H-benzimidazol-6-yl)- 2,3,4,5-tetrahydro-1,4- benzoxazepine |
| 18 | 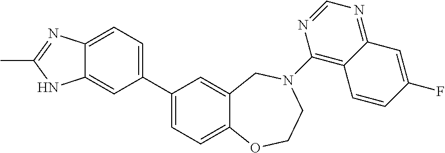 [SMILES: Cc1nc2ccc(-c3ccc4c(c3)CN(c3ncnc5cc(F)ccc35)CCO4)cc2[nH]1] | 4-(7-fluoroquinazolin-4-yl)-7-(2- methyl-1H-benzimidazol-6-yl)- 2,3,4,5-tetrahydro-1,4- benzoxazepine |
| 19 | 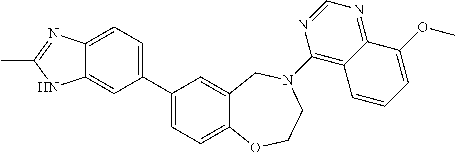 | 7-(2-methyl-1H-benzimidazol-6- yl)-4-[8-(methyloxy)quinazolin- 4-yl]-2,3,4,5-tetrahydro-1,4- benzoxazepine |
| 20 | 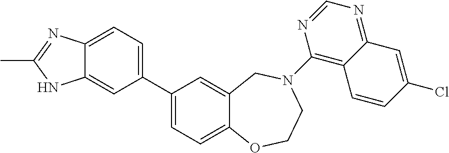 [SMILES: Cc1nc2ccc(-c3ccc4c(c3)CN(c3ncnc5cc(Cl)ccc35)CCO4)cc2[nH]1] | 4-(7-chloroquinazolin-4-yl)-7- (2-methyl-1H-benzimidazol-6- yl)-2,3,4,5-tetrahydro-1,4- benzoxazepine |
| 21 | 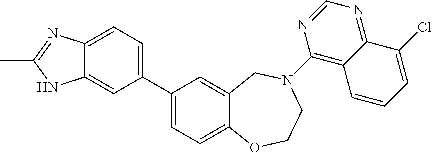 [SMILES: Cc1nc2ccc(-c3ccc4c(c3)CN(c3ncnc5c(Cl)cccc35)CCO4)cc2[nH]1] | 4-(8-chloroquinazolin-4-yl)-7- (2-methyl-1H-benzimidazol-6- yl)-2,3,4,5-tetrahydro-1,4- benzoxazepine |
| 22 | 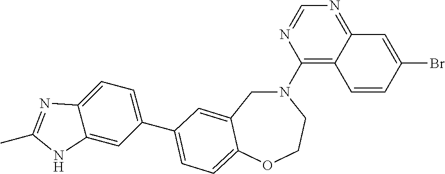 [SMILES: Cc1nc2ccc(-c3ccc4c(c3)CN(c3ncnc5cc(Br)ccc35)CCO4)cc2[nH]1] | 4-(7-bromoquinazolin-4-yl)-7- (2-methyl-1H-benzimidazol-6- yl)-2,3,4,5-tetrahydro-1,4- benzoxazepine |
| 23 | 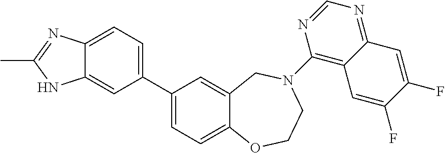 [SMILES: Cc1nc2ccc(-c3ccc4c(c3)CN(c3ncnc5cc(F)c(F)cc35)CCO4)cc2[nH]1] | 4-(6,7-difluoroquinazolin-4-yl)- 7-(2-methyl-1H-benzimidazol-6- yl)-2,3,4,5-tetrahydro-1,4- benzoxazepine |
| 24 | 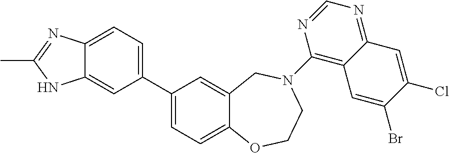 [SMILES: Cc1nc2ccc(-c3ccc4c(c3)CN(c3ncnc5cc(Cl)c(Br)cc35)CCO4)cc2[nH]1] | 4-(6-bromo-7-chloroquinazolin- 4-yl)-7-(2-methyl-1H- benzimidazol-6-yl)-2,3,4,5- tetrahydro-1,4-benzoxazepine |
| 25 | 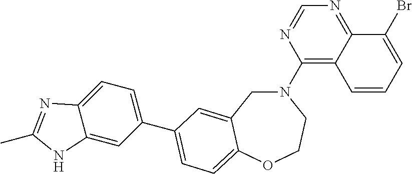 [SMILES: Cc1nc2ccc(-c3ccc4c(c3)CN(c3ncnc5c(Br)cccc35)CCO4)cc2[nH]1] | 4-(8-bromoquinazolin-4-yl)-7- (2-methyl-1H-benzimidazol-6- yl)-2,3,4,5-tetrahydro-1,4- benzoxazepine |
| 26 | 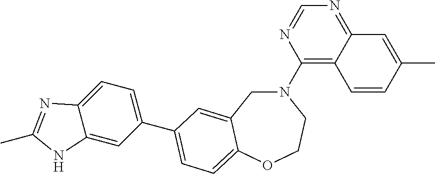 | 7-(2-methyl-1H-benzimidazol-6- yl)-4-(7-methylquinazolin-4-yl)- 2,3,4,5-tetrahydro-1,4- benzoxazepine |
| 27 | 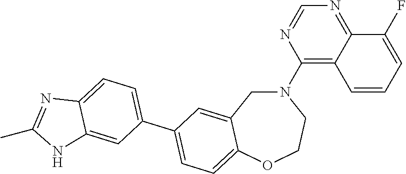 [SMILES: Cc1nc2ccc(-c3ccc4c(c3)CN(c3ncnc5c(F)cccc35)CCO4)cc2[nH]1] | 4-(8-fluoroquinazolin-4-yl)-7-(2- methyl-1H-benzimidazol-6-yl)- 2,3,4,5-tetrahydro-1,4- benzoxazepine |
| 28 | 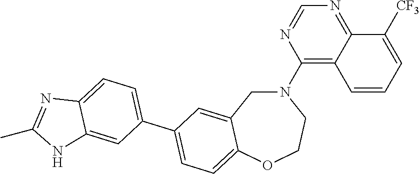 [SMILES: Cc1nc2ccc(-c3ccc4c(c3)CN(c3ncnc5c(C(F)(F)F)cccc35)CCO4)cc2[nH]1] | 7-(2-methyl-1H-benzimidazol-6- yl)-4-[8- (trifluoromethyl)quinazolin-4- yl]-2,3,4,5-tetrahydro-1,4- benzoxazepine |
| 29 | 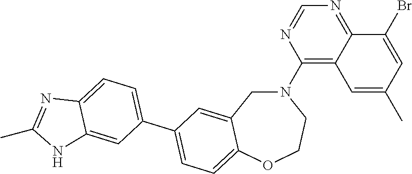 | 4-(8-bromo-6-methylquinazolin- 4-yl)-7-(2-methyl-1H- benzimidazol-6-yl)-2,3,4,5- tetrahydro-1,4-benzoxazepine |
| 30 | 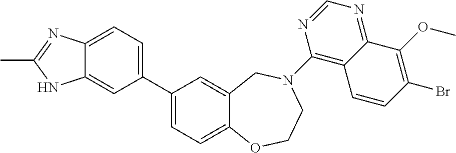 | 4-[7-bromo-8- (methyloxy)quinazolin-4-yl]-7- (2-methyl-1H-benzimidazol-6- yl)-2,3,4,5-tetrahydro-1,4- benzoxazepine |
| 31 | 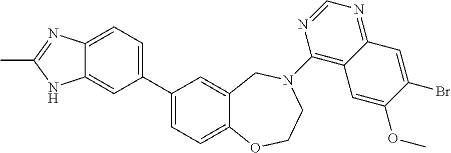 [SMILES: COc1cc2c(N3CCOc4ccc(-c5ccc6nc(C)[nH]c6c5)cc4C3)ncnc2cc1Br] | 4-[7-bromo-6- (methyloxy)quinazolin-4-yl]-7- (2-methyl-1H-benzimidazol-6- yl)-2,3,4,5-tetrahydro-1,4- benzoxazepine |
| 32 | 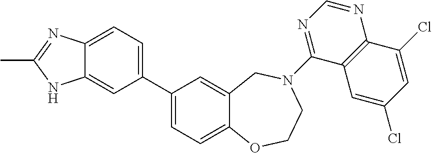 [SMILES: Cc1nc2ccc(-c3ccc4c(c3)CN(c3ncnc5c(Cl)cc(Cl)cc35)CCO4)cc2[nH]1] | 4-(6,8-dichloroquinazolin-4-yl)- 7-(2-methyl-1H-benzimidazol-6- yl)-2,3,4,5-tetrahydro-1,4- benzoxazepine |
| 33 | 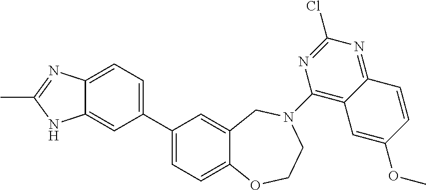 | 4-[2-chloro-6- (methyloxy)quinazolin-4-yl]-7- (2-methyl-1H-benzimidazol-6- yl)-2,3,4,5-tetrahydro-1,4- benzoxazepine |
| 34 | 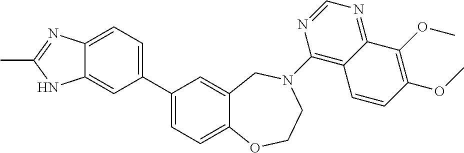 | 4-[7,8- bis(methyloxy)quinazolin-4-yl]- 7-(2-methyl-1H-benzimidazol-6- yl)-2,3,4,5-tetrahydro-1,4- benzoxazepine |
| 35 | 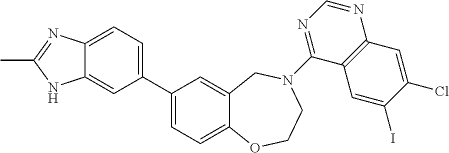 [SMILES: Cc1nc2ccc(-c3ccc4c(c3)CN(c3ncnc5cc(Cl)c(I)cc35)CCO4)cc2[nH]1] | 4-(7-chloro-6-iodoquinazolin-4- yl)-7-(2-methyl-1H- benzimidazol-6-yl)-2,3,4,5- tetrahydro-1,4-benzoxazepine |
| 36 | 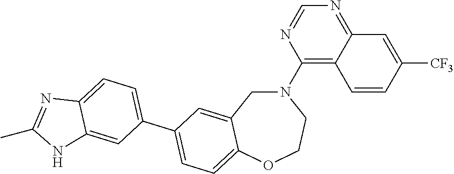 [SMILES: Cc1nc2ccc(-c3ccc4c(c3)CN(c3ncnc5cc(C(F)(F)F)ccc35)CCO4)cc2[nH]1] | 7-(2-methyl-1H-benzimidazol-6- yl)-4-[7- (trifluoromethyl)quinazolin-4- yl]-2,3,4,5-tetrahydro-1,4- benzoxazepine |
| 37 | 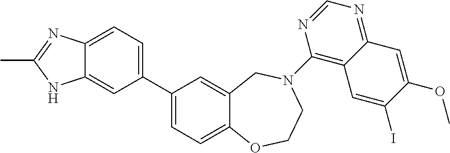 | 4-[6-iodo-7- (methyloxy)quinazolin-4-yl]-7- (2-methyl-1H-benzimidazol-6- yl)-2,3,4,5-tetrahydro-1,4- benzoxazepine |
| 38 | 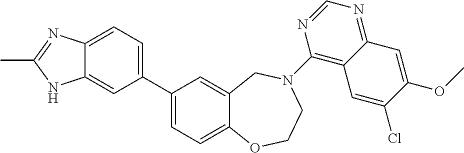 | 4-[6-chloro-7- (methyloxy)quinazolin-4-yl]-7- (2-methyl-1H-benzimidazol-6- yl)-2,3,4,5-tetrahydro-1,4- benzoxazepine |
| 39 | 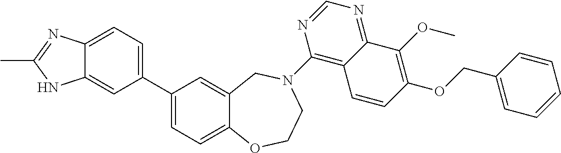 [SMILES: COc1c(OCc2ccccc2)ccc2c(N3CCOc4ccc(-c5ccc6nc(C)[nH]c6c5)cc4C3)ncnc12] | 7-(2-methyl-1H-benzimidazol-6- yl)-4-{8-(methyloxy)-7- [(phenylmethyl)oxy]quinazolin- 4-yl}-2,3,4,5-tetrahydro-1,4- benzoxazepine |
| 40 | 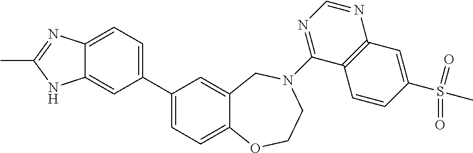 | 7-(2-methyl-1H-benzimidazol-6- yl)-4-[7- (methylsulfonyl)quinazolin-4- yl]-2,3,4,5-tetrahydro-1,4- benzoxazepine |
| 41 | 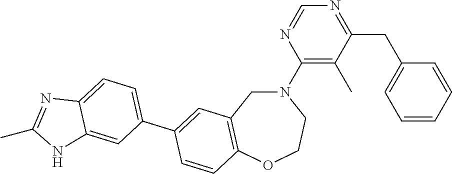 [SMILES: Cc1nc2ccc(-c3ccc4c(c3)CN(c3ncnc(Cc5ccccc5)c3C)CCO4)cc2[nH]1] | 7-(2-methyl-1H-benzimidazol-6- yl)-4-[5-methyl-6- (phenylmethyl)pyrimidin-4-yl]- 2,3,4,5-tetrahydro-1,4- benzoxazepine |
| 42 | 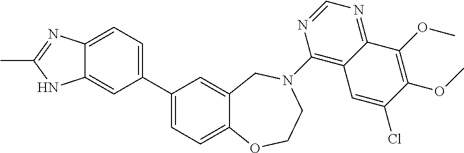 | 4-[6-chloro-7,8- bis(methyloxy)quinazolin-4-yl]- 7-(2-methyl-1H-benzimidazol-6- yl)-2,3,4,5-tetrahydro-1,4- benzoxazepine |
| 43 | 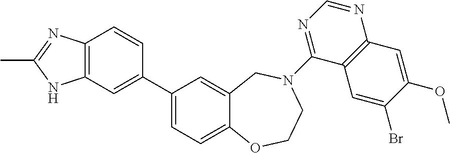 | 4-[6-bromo-7- (methyloxy)quinazolin-4-yl]-7- (2-methyl-1H-benzimidazol-6- yl)-2,3,4,5-tetrahydro-1,4- benzoxazepine |
| 44 | 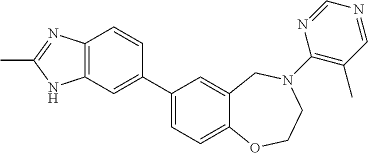 [SMILES: Cc1nc2ccc(-c3ccc4c(c3)CN(c3ncncc3C)CCO4)cc2[nH]1] | 7-(2-methyl-1H-benzimidazol-6- yl)-4-(5-methylpyrimidin-4-yl)- 2,3,4,5-tetrahydro-1,4- benzoxazepine |
| 45 | 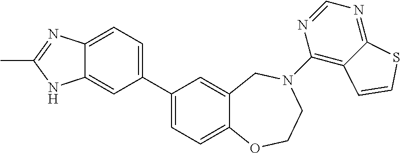 [SMILES: Cc1nc2ccc(-c3ccc4c(c3)CN(c3ncnc5sccc35)CCO4)cc2[nH]1] | 7-(2-methyl-1H-benzimidazol-6- yl)-4-thieno[2,3-d]pyrimidin-4- yl-2,3,4,5-tetrahydro-1,4- benzoxazepine |
| 46 | 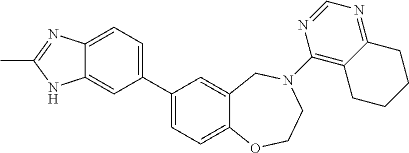 [SMILES: Cc1nc2ccc(-c3ccc4c(c3)CN(c3ncnc5c3CCCC5)CCO4)cc2[nH]1] | 7-(2-methyl-1H-benzimidazol-6- yl)-4-(5,6,7,8- tetrahydroquinazolin-4-yl)- 2,3,4,5-tetrahydro-1,4- benzoxazepine |
| 47 | 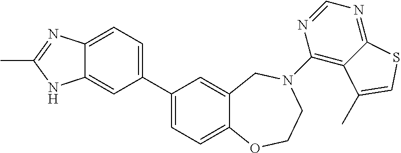 [SMILES: Cc1nc2ccc(-c3ccc4c(c3)CN(c3ncnc5scc(C)c35)CCO4)cc2[nH]1] | 7-(2-methyl-1H-benzimidazol-6- yl)-4-(5-methylthieno[2,3- d]pyrimidin-4-yl)-2,3,4,5- tetrahydro-1,4-benzoxazepine |
| 48 | 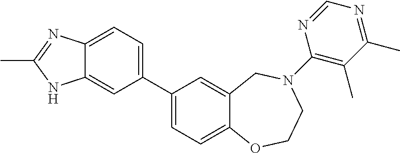 | 4-(5,6-dimethylpyrimidin-4-yl)- 7-(2-methyl-1H-benzimidazol-6- yl)-2,3,4,5-tetrahydro-1,4- benzoxazepine |
| 49 | 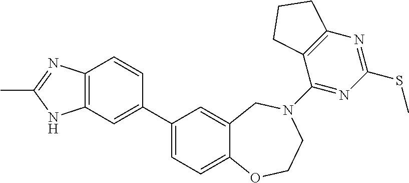 | 7-(2-methyl-1H-benzimidazol-6- yl)-4-[2-(methylthio)-6,7- dihydro-5H- cyclopenta[d]pyrimidin-4-yl]- 2,3,4,5-tetrahydro-1,4- benzoxazepine |
| 50 | 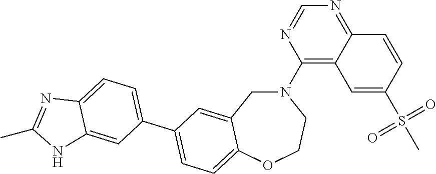 [SMILES: Cc1nc2ccc(-c3ccc4c(c3)CN(c3ncnc5ccc(S(C)(=O)=O)cc35)CCO4)cc2[nH]1] | 7-(2-methyl-1H-benzimidazol-6- yl)-4-[6- (methylsulfonyl)quinazolin-4- yl]-2,3,4,5-tetrahydro-1,4- benzoxazepine |
| 51 | 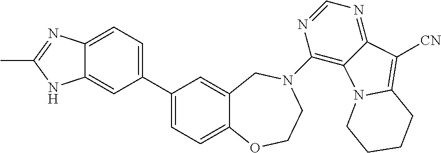 [SMILES: Cc1nc2ccc(-c3ccc4c(c3)CN(c3ncnc5c(C#N)c6n(c35)CCCC6)CCO4)cc2[nH]1] | 4-[7-(2-methyl-1H- benzimidazol-6-yl)-2,3-dihydro- 1,4-benzoxazepin-4(5H)-yl]- 6,7,8,9-tetrahydropyrimido[4,5- b]indolizine-10-carbonitrile |
| 52 | 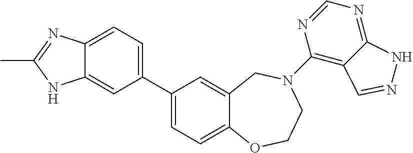 [SMILES: Cc1nc2ccc(-c3ccc4c(c3)CN(c3ncnc5[nH]ncc35)CCO4)cc2[nH]1] | 7-(2-methyl-1H-benzimidazol-6- yl)-4-(1H-pyrazolo[3,4- d]pyrimidin-4-yl)-2,3,4,5- tetrahydro-1,4-benzoxazepine |
| 53 | 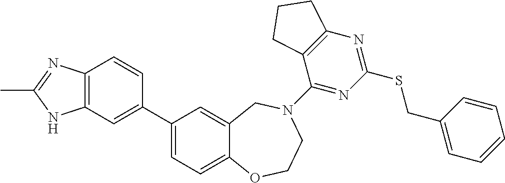 [SMILES: Cc1nc2ccc(-c3ccc4c(c3)CN(c3nc(SCc5ccccc5)nc5c3CCC5)CCO4)cc2[nH]1] | 7-(2-methyl-1H-benzimidazol-6- yl)-4-{2-[(phenylmethyl)thio]- 6,7-dihydro-5H- cyclopenta[d]pyrimidin-4-yl}- 2,3,4,5-tetrahydro-1,4- benzoxazepine |
| 54 | 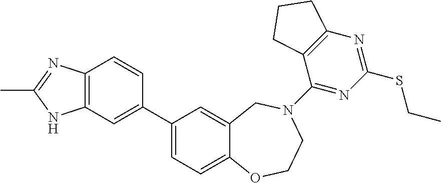 [SMILES: CCSc1nc2c(c(N3CCOc4ccc(-c5ccc6nc(C)[nH]c6c5)cc4C3)n1)CCC2] | 4-[2-(ethylthio)-6,7-dihydro-5H- cyclopenta[d]pyrimidin-4-yl]-7- (2-methyl-1H-benzimidazol-6- yl)-2,3,4,5-tetrahydro-1,4- benzoxazepine |
| 55 | 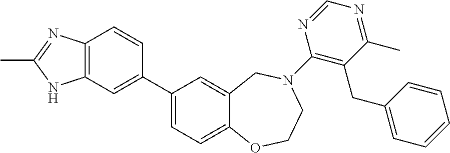 [SMILES: Cc1nc2ccc(-c3ccc4c(c3)CN(c3ncnc(C)c3Cc3ccccc3)CCO4)cc2[nH]1] | 7-(2-methyl-1H-benzimidazol-6- yl)-4-[6-methyl-5- (phenylmethyl)pyrimidin-4-yl]- 2,3,4,5-tetrahydro-1,4- benzoxazepine |
| 56 | 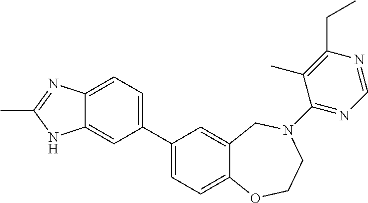 [SMILES: CCc1ncnc(N2CCOc3ccc(-c4ccc5nc(C)[nH]c5c4)cc3C2)c1C] | 4-(6-ethyl-5-methylpyrimidin-4- yl)-7-(2-methyl-1H- benzimidazol-6-yl)-2,3,4,5- tetrahydro-1,4-benzoxazepine |
| 57 | 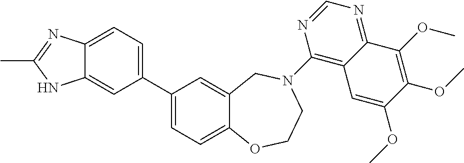 | 7-(2-methyl-1H-benzimidazol-6- yl)-4-[6,7,8- tris(methyloxy)quinazolin-4-yl]- 2,3,4,5-tetrahydro-1,4- benzoxazepine |
| 58 | 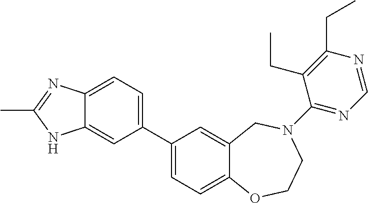 [SMILES: CCc1ncnc(N2CCOc3ccc(-c4ccc5nc(C)[nH]c5c4)cc3C2)c1CC] | 4-(5,6-diethylpyrimidin-4-yl)-7- (2-methyl-1H-benzimidazol-6- yl)-2,3,4,5-tetrahydro-1,4- benzoxazepine |
| 59 | 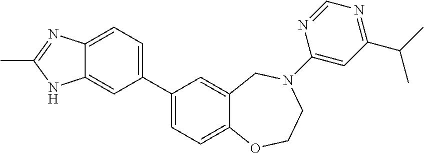 [SMILES: Cc1nc2ccc(-c3ccc4c(c3)CN(c3cc(C(C)C)ncn3)CCO4)cc2[nH]1] | 7-(2-methyl-1H-benzimidazol-6- yl)-4-[6-(1- methylethyl)pyrimidin-4-yl]- 2,3,4,5-tetrahydro-1,4- benzoxazepine |
| 60 | 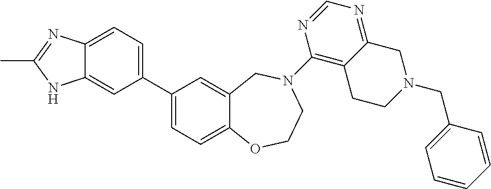 [SMILES: Cc1nc2ccc(-c3ccc4c(c3)CN(c3ncnc5c3CCN(Cc3ccccc3)C5)CCO4)cc2[nH]1] | 7-(2-methyl-1H-benzimidazol-6- yl)-4-[7-(phenylmethyl)-5,6,7,8- tetrahydropyrido[3,4- d]pyrimidin-4-yl]-2,3,4,5- tetrahydro-1,4-benzoxazepine |
| 61 | 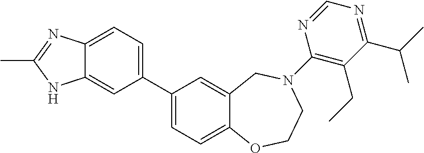 [SMILES: CCc1c(C(C)C)ncnc1N1CCOc2ccc(-c3ccc4nc(C)[nH]c4c3)cc2C1] | 4-[5-ethyl-6-(1- methylethyl)pyrimidin-4-yl]-7- (2-methyl-1H-benzimidazol-6- yl)-2,3,4,5-tetrahydro-1,4- benzoxazepine |
| 62 | 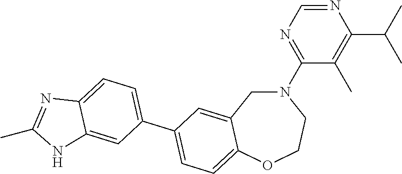 [SMILES: Cc1nc2ccc(-c3ccc4c(c3)CN(c3ncnc(C(C)C)c3C)CCO4)cc2[nH]1] | 7-(2-methyl-1H-benzimidazol-6- yl)-4-[5-methyl-6-(1- methylethyl)pyrimidin-4-yl]- 2,3,4,5-tetrahydro-1,4- benzoxazepine |
| 63 | 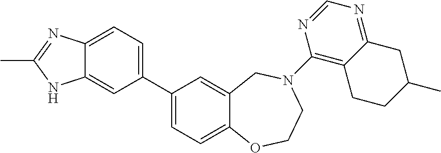 | 7-(2-methyl-1H-benzimidazol-6- yl)-4-(7-methyl-5,6,7,8- tetrahydroquinazolin-4-yl)- 2,3,4,5-tetrahydro-1,4- benzoxazepine |
| 64 | 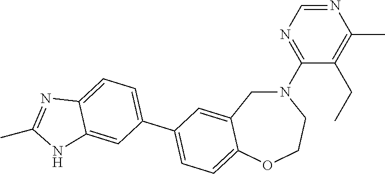 | 4-(5-ethyl-6-methylpyrimidin-4- yl)-7-(2-methyl-1H- benzimidazol-6-yl)-2,3,4,5- tetrahydro-1,4-benzoxazepine |
| 65 | 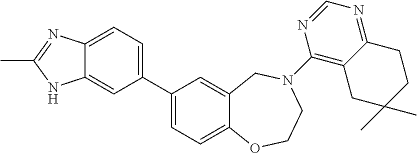 | 4-(6,6-dimethyl-5,6,7,8- tetrahydroquinazolin-4-yl)-7-(2- methyl-1H-benzimidazol-6-yl)- 2,3,4,5-tetrahydro-1,4- benzoxazepine |
| 66 | 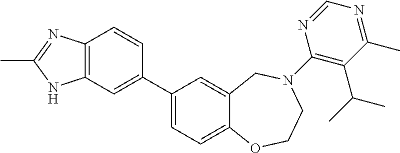 | 7-(2-methyl-1H-benzimidazol-6- yl)-4-[6-methyl-5-(1- methylethyl)pyrimidin-4-yl]- 2,3,4,5-tetrahydro-1,4- benzoxazepine |
| 67 | 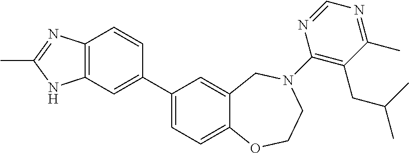 [SMILES: Cc1nc2ccc(-c3ccc4c(c3)CN(c3ncnc(C)c3CC(C)C)CCO4)cc2[nH]1] | 7-(2-methyl-1H-benzimidazol-6- yl)-4-[6-methyl-5-(2- methylpropyl)pyrimidin-4-yl]- 2,3,4,5-tetrahydro-1,4- benzoxazepine |
| 68 | 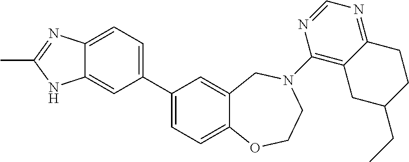 [SMILES: CCC1CCc2ncnc(N3CCOc4ccc(-c5ccc6nc(C)[nH]c6c5)cc4C3)c2C1] | 4-(6-ethyl-5,6,7,8- tetrahydroquinazolin-4-yl)-7-(2- methyl-1H-benzimidazol-6-yl)- 2,3,4,5-tetrahydro-1,4- benzoxazepine |
| 69 | 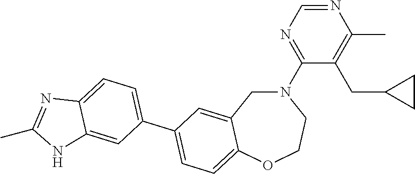 [SMILES: Cc1nc2ccc(-c3ccc4c(c3)CN(c3ncnc(C)c3CC3CC3)CCO4)cc2[nH]1] | 4-[5-(cyclopropylmethyl)-6- methylpyrimidin-4-yl)-7-(2- methyl-1H-benzimidazol-6-yl)- 2,3,4,5-tetrahydro-1,4- benzoxazepine |
| 70 | 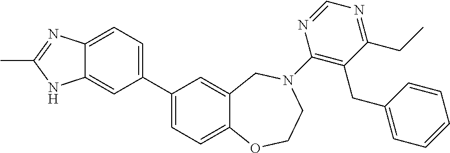 [SMILES: CCc1ncnc(N2CCOc3ccc(-c4ccc5nc(C)[nH]c5c4)cc3C2)c1Cc1ccccc1] | 4-[6-ethyl-5- (phenylmethyl)pyrimidin-4-yl]- 7-(2-methyl-1H-benzimidazol-6- yl)-2,3,4,5-tetrahydro-1,4- benzoxazepine |
| 71 | 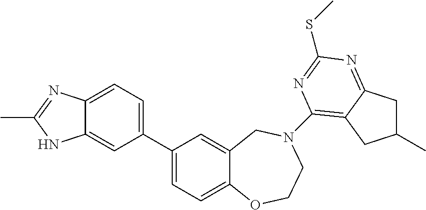 | 7-(2-methyl-1H-benzimidazol-6- yl)-4-[6-methyl-2-(methylthio)- 6,7-dihydro-5H- cyclopenta[d]pyrimidin-4-yl]- 2,3,4,5-tetrahydro-1,4- benzoxazepine |
| 72 | 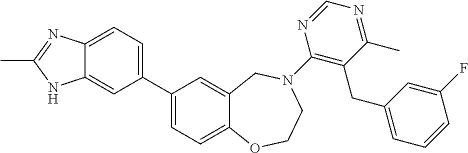 [SMILES: Cc1nc2ccc(-c3ccc4c(c3)CN(c3ncnc(C)c3Cc3cccc(F)c3)CCO4)cc2[nH]1] | 4-{5-[(3-fluorophenyl)methyl]- 6-methylpyrimidin-4-yl}-7-(2- methyl-1H-benzimidazol-6-yl)- 2,3,4,5-tetrahydro-1,4- benzoxazepine |
| 73 | 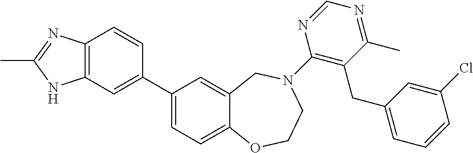 [SMILES: Cc1nc2ccc(-c3ccc4c(c3)CN(c3ncnc(C)c3Cc3cccc(Cl)c3)CCO4)cc2[nH]1] | 4-{5-[(3-chlorophenyl)methyl]- 6-methylpyrimidin-4-yl}-7-(2- methyl-1H-benzimidazol-6-yl)- 2,3,4,5-tetrahydro-1,4- benzoxazepine |
| 74 | 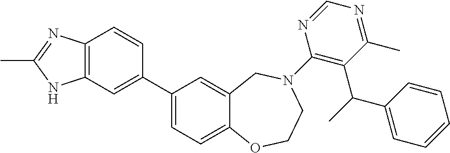 [SMILES: Cc1nc2ccc(-c3ccc4c(c3)CN(c3ncnc(C)c3C(C)c3ccccc3)CCO4)cc2[nH]1] | 7-(2-methyl-1H-benzimidazol-6- yl)-4-[6-methyl-5-(1- phenylethyl)pyrimidin-4-yl]- 2,3,4,5-tetrahydro-1,4- benzoxazepine |
| 75 | 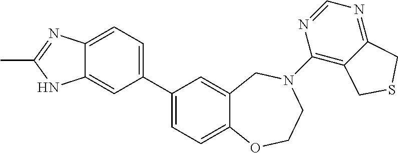 [SMILES: Cc1nc2ccc(-c3ccc4c(c3)CN(c3ncnc5c3CSC5)CCO4)cc2[nH]1] | 4-(5,7-dihydrothieno[3,4- d]pyrimidin-4-yl)-7-(2-methyl- 1H-benzimidazol-6-yl)-2,3,4,5- tetrahydro-1,4-benzoxazepine |
| 76 | 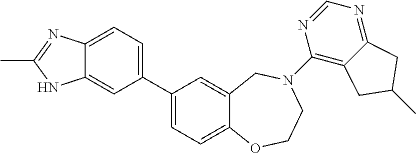 | 7-(2-methyl-1H-benzimidazol-6- yl)-4-(6-methyl-6,7-dihydro-5H- cyclopenta[d]pyrimidin-4-yl)- 2,3,4,5-tetrahydro-1,4- benzoxazepine |
| 77 | 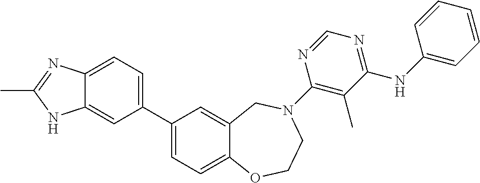 [SMILES: Cc1nc2ccc(-c3ccc4c(c3)CN(c3ncnc(Nc5ccccc5)c3C)CCO4)cc2[nH]1] | 5-methyl-6-[7-(2-methyl-1H- benzimidazol-6-yl)-2,3-dihydro- 1,4-benzoxazepin-4(5H)-yl]-N- phenylpyrimidin-4-amine |
| 78 | 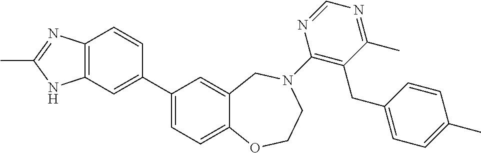 | 7-(2-methyl-1H-benzimidazol-6- yl)-4-{6-methyl-5-[(4- methylphenyl)methyl]pyrimidin- 4-yl}-2,3,4,5-tetrahydro-1,4- benzoxazepine |
| 79 | 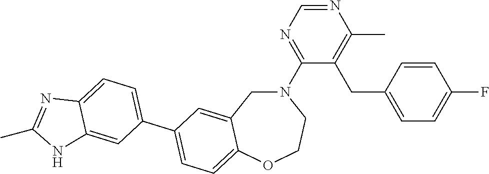 [SMILES: Cc1nc2ccc(-c3ccc4c(c3)CN(c3ncnc(C)c3Cc3ccc(F)cc3)CCO4)cc2[nH]1] | 4-{5-[(4-fluorophenyl)methyl]- 6-methylpyrimidin-4-yl}-7-(2- methyl-1H-benzimidazol-6-yl)- 2,3,4,5-tetrahydro-1,4- benzoxazepine |
| 80 | 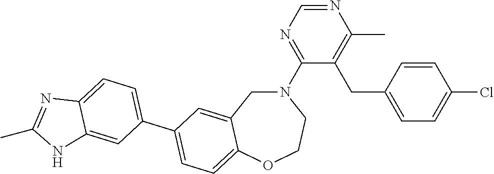 [SMILES: Cc1nc2ccc(-c3ccc4c(c3)CN(c3ncnc(C)c3Cc3ccc(Cl)cc3)CCO4)cc2[nH]1] | 4-{5-[(4-chlorophenyl)methyl]- 6-methylpyrimidin-4-yl}-7-(2- methyl-1H-benzimidazol-6-yl)- 2,3,4,5-tetrahydro-1,4- benzoxazepine |
| 81 | 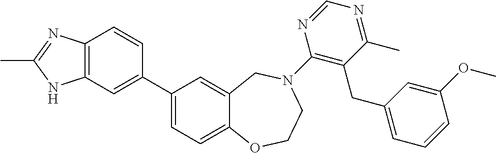 | 7-(2-methyl-1H-benzimidazol-6- yl)-4-(6-methyl-5-{[3- (methyloxy)phenyl]methyl} pyrimidin-4-yl)-2,3,4,5- tetrahydro-1,4-benzoxazepine |
| 82 | 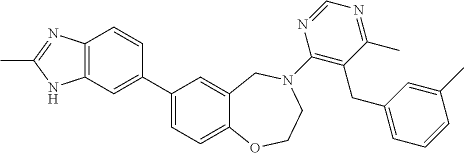 | 7-(2-methyl-1H-benzimidazol-6- yl)-4-(6-methyl-5-[(3- methylphenyl)methyl]pyrimidin- 4-yl}-2,3,4,5-tetrahydro-1,4- benzoxazepine |
| 83 | 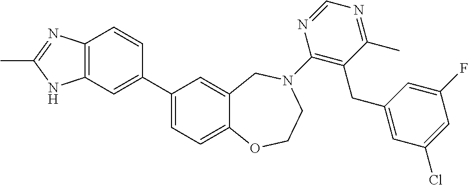 [SMILES: Cc1nc2ccc(-c3ccc4c(c3)CN(c3ncnc(C)c3Cc3cc(F)cc(Cl)c3)CCO4)cc2[nH]1] | 4-{5-[(3-chloro-5- fluorophenyl)methyl]-6- methylpyrimidin-4-yl}-7-(2- methyl-1H-benzimidazol-6-yl)- 2,3,4,5-tetrahydro-1,4- benzoxazepine |
| 84 | 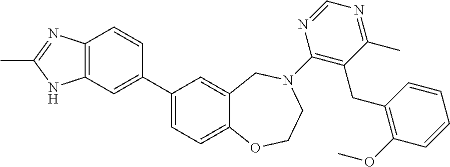 [SMILES: COc1ccccc1Cc1c(C)ncnc1N1CCOc2ccc(-c3ccc4nc(C)[nH]c4c3)cc2C1] | 7-(2-methyl-1H-benzimidazol-6- yl)-4-(6-methyl-5-{[2- (methyloxy)phenyl]methyl} pyrimidin-4-yl)-2,3,4,5-tetrahydro-1,4-benzoxazepine |
| 85 | 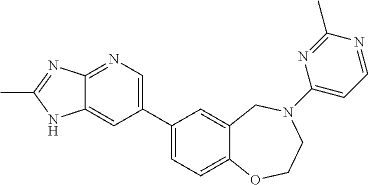 [SMILES: Cc1nccc(N2CCOc3ccc(-c4cnc5nc(C)[nH]c5c4)cc3C2)n1] | 7-(2-methyl-1H-imidazo[4,5- b]pyridin-6-yl)-4-(2- methylpyrimidin-4-yl)-2,3,4,5- tetrahydro-1,4-benzoxazepine |
| 86 | 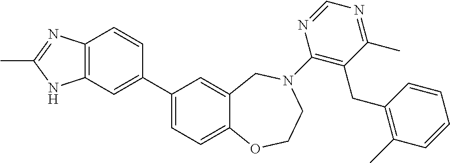 [SMILES: Cc1nc2ccc(-c3ccc4c(c3)CN(c3ncnc(C)c3Cc3ccccc3C)CCO4)cc2[nH]1] | 7-(2-methyl-1H-benzimidazol-6- yl)-4-{6-methyl-5-[(2- methylphenyl)methyl]pyrimidin- 4-yl}-2,3,4,5-tetrahydro-1,4- benzoxazepine |
| 87 | 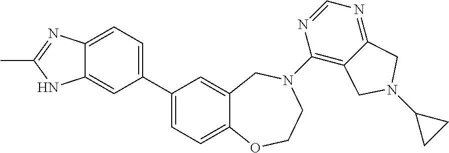 [SMILES: Cc1nc2ccc(-c3ccc4c(c3)CN(c3ncnc5c3CN(C3CC3)C5)CCO4)cc2[nH]1] | 4-(6-cyclopropyl-6,7-dihydro- 5H-pyrrolo[3,4-d]pyrimidin-4- yl)-7-(2-methyl-1H- benzimidazol-6-yl)-2,3,4,5- tetrahydro-1,4-benzoxazepine |
| 88 | 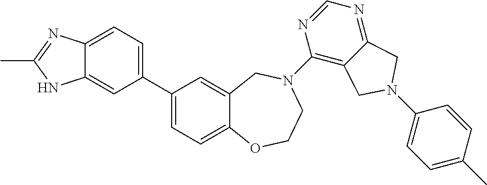 | 7-(2-methyl-1H-benzimidazol-6- yl)-4-[6-(4-methylphenyl)-6,7- dihydro-5H-pyrrolo[3,4- d]pyrimidin-4-yl]-2,3,4,5- tetrahydro-1,4-benzoxazepine |
| 89 | 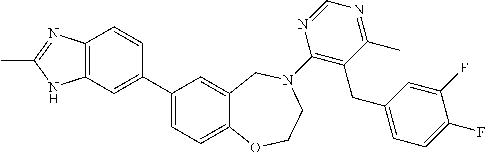 [SMILES: Cc1nc2ccc(-c3ccc4c(c3)CN(c3ncnc(C)c3Cc3ccc(F)c(F)c3)CCO4)cc2[nH]1] | 4-{5-[(3,4- difluorophenyl)methyl]-6- methylpyrimidin-4-yl}-7-(2- methyl-1H-benzimidazol-6-yl)- 2,3,4,5-tetrahydro-1,4- benzoxazepine |
| 90 | 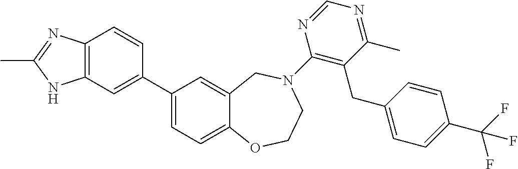 [SMILES: Cc1nc2ccc(-c3ccc4c(c3)CN(c3ncnc(C)c3Cc3ccc(C(F)(F)F)cc3)CCO4)cc2[nH]1] | 7-(2-methyl-1H-benzimidazol-6- yl)-4-(6-methyl-5-{[4- (trifluoromethyl)phenyl]methyl) pyrimidin-4-yl)-2,3,4,5- tetrahydro-1,4-benzoxazepine |
| 91 | 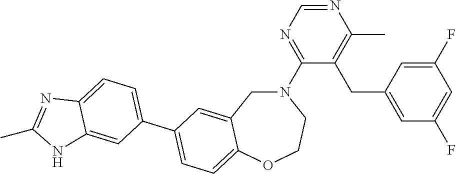 [SMILES: Cc1nc2ccc(-c3ccc4c(c3)CN(c3ncnc(C)c3Cc3cc(F)cc(F)c3)CCO4)cc2[nH]1] | 4-{5-[(3,5- difluorophenyl)methyl]-6- methylpyrimidin-4-yl}-7-(2- methyl-1H-benzimidazol-6-yl)- 2,3,4,5-tetrahydro-1,4- benzoxazepine |
| 92 | 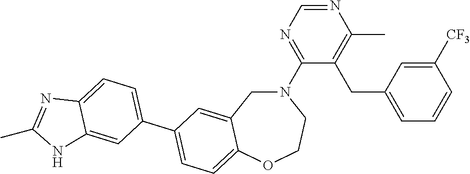 [SMILES: Cc1nc2ccc(-c3ccc4c(c3)CN(c3ncnc(C)c3Cc3cccc(C(F)(F)F)c3)CCO4)cc2[nH]1] | 7-(2-methyl-1H-benzimidazol-6- yl)-4-(6-methyl-5-{[3- (trifluoromethyl)phenyl]methyl} pyrimidin-4-yl)-2,3,4,5- tetrahydro-1,4-benzoxazepine |
| 93 | 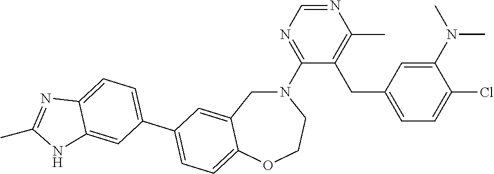 [SMILES: Cc1nc2ccc(-c3ccc4c(c3)CN(c3ncnc(C)c3Cc3ccc(Cl)c(N(C)C)c3)CCO4)cc2[nH]1] | 2-chloro-N,N-dimethyl-5-({4- methyl-6-[7-(2-methyl-1H- benzimidazol-6-yl)-2,3-dihydro- 1,4-benzoxazepin-4(5H)- yl]pyrimidin-5-yl}methyl)aniline |
| 94 | 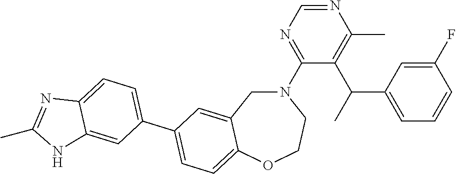 [SMILES: Cc1nc2ccc(-c3ccc4c(c3)CN(c3ncnc(C)c3C(C)c3cccc(F)c3)CCO4)cc2[nH]1] | 4-{5-[1-(3-fluorophenyl)ethyl]- 6-methylpyrimidin-4-yl}-7-(2- methyl-1H-benzimidazol-6-yl)- 2,3,4,5-tetrahydro-1,4- benzoxazepine |
| 95 | 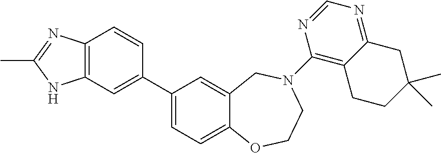 | 4-(7,7-dimethyl-5,6,7,8- tetrahydroquinazolin-4-yl)-7-(2- methyl-1H-benzimidazol-6-yl)- 2,3,4,5-tetrahydro-1,4- benzoxazepine |
| 96 | 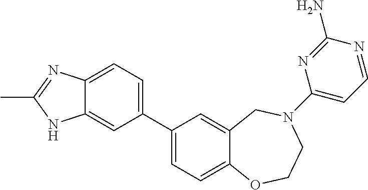 [SMILES: Cc1nc2ccc(-c3ccc4c(c3)CN(c3ccnc(N)n3)CCO4)cc2[nH]1] | 4-[7-(2-methyl-1H- benzimidazol-6-yl)-2,3-dihydro- 1,4-benzoxazepin-4(5H)- yl]pyrimidin-2-amine |
| 97 | 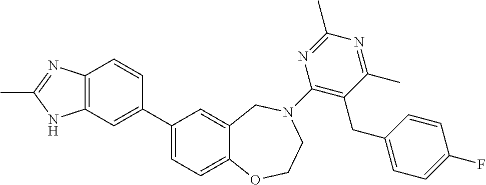 [SMILES: Cc1nc(C)c(Cc2ccc(F)cc2)c(N2CCOc3ccc(-c4ccc5nc(C)[nH]c5c4)cc3C2)n1] | 4-{5-[(4-fluorophenyl)methyl]- 2,6-dimethylpyrimidin-4-yl}-7- (2-methyl-1H-benzimidazol-6- yl)-2,3,4,5-tetrahydro-1,4- benzoxazepine |
| 98 | 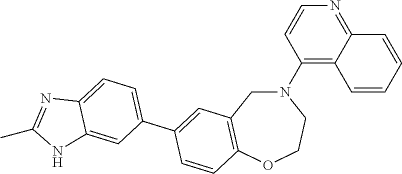 [SMILES: Cc1nc2ccc(-c3ccc4c(c3)CN(c3ccnc5ccccc35)CCO4)cc2[nH]1] | 7-(2-methyl-1H-benzimidazol-6- yl)-4-quinolin-4-yl-2,3,4,5- tetrahydro-1,4-benzoxazepine |
| 99 | 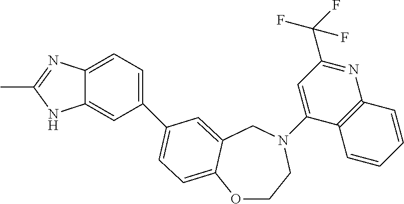 [SMILES: Cc1nc2ccc(-c3ccc4c(c3)CN(c3cc(C(F)(F)F)nc5ccccc35)CCO4)cc2[nH]1] | 7-(2-methyl-1H-benzimidazol-6- yl)-4-[2- (trifluoromethyl)quinolin-4-yl]- 2,3,4,5-tetrahydro-1,4- benzoxazepine |
| 100 | 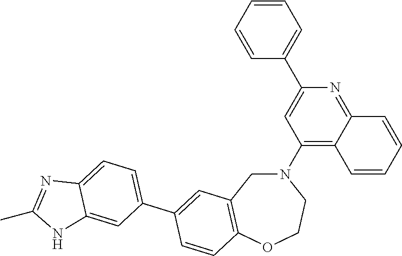 [SMILES: Cc1nc2ccc(-c3ccc4c(c3)CN(c3cc(-c5ccccc5)nc5ccccc35)CCO4)cc2[nH]1] | 7-(2-methyl-1H-benzimidazol-6- yl)-4-(2-phenylquinolin-4-yl)- 2,3,4,5-tetrahydro-1,4- benzoxazepine |
| 101 | 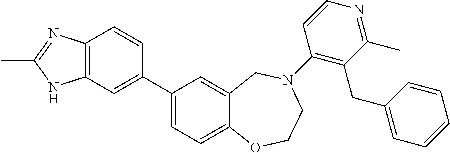 [SMILES: Cc1nc2ccc(-c3ccc4c(c3)CN(c3ccnc(C)c3Cc3ccccc3)CCO4)cc2[nH]1] | 7-(2-methyl-1H-benzimidazol-6- yl)-4-[2-methyl-3- (phenylmethyl)pyridin-4-yl]- 2,3,4,5-tetrahydro-1,4- benzoxazepine |
| 102 | 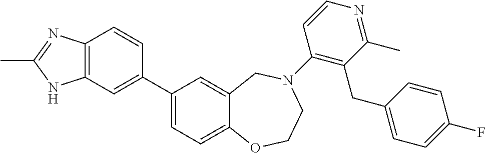 [SMILES: Cc1nc2ccc(-c3ccc4c(c3)CN(c3ccnc(C)c3Cc3ccc(F)cc3)CCO4)cc2[nH]1] | 4-{3-[(4-fluorophenyl)methyl]- 2-methylpyridin-4-yl}-7-(2- methyl-1H-benzimidazol-6-yl)- 2,3,4,5-tetrahydro-1,4- benzoxazepine |
| 103 | 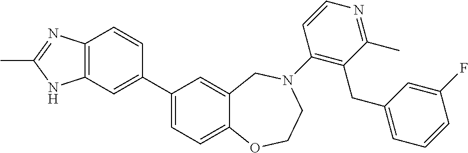 [SMILES: Cc1nc2ccc(-c3ccc4c(c3)CN(c3ccnc(C)c3Cc3cccc(F)c3)CCO4)cc2[nH]1] | 4-{3-[(4-fluorophenyl)methyl]- 2-methylpyridin-4-yl}-7-(2- methyl-1H-benzimidazol-6-yl)- 2,3,4,5-tetrahydro-1,4- benzoxazepine |
| 104 | 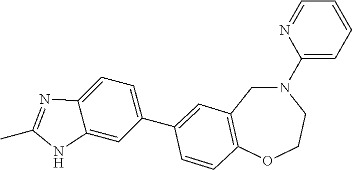 [SMILES: Cc1nc2ccc(-c3ccc4c(c3)CN(c3ccccn3)CCO4)cc2[nH]1] | 7-(2-methyl-1H-benzimidazol-6- yl)-4-pyridin-2-yl-2,3,4,5- tetrahydro-1,4-benzoxazepine |
| 105 | 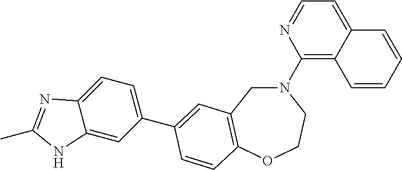 [SMILES: Cc1nc2ccc(-c3ccc4c(c3)CN(c3nccc5ccccc35)CCO4)cc2[nH]1] | 4-isoquinolin-1-yl-7-(2-methyl- 1H-benzimidazol-6-yl)-2,3,4,5- tetrahydro-1,4-benzoxazepine |
| 106 | 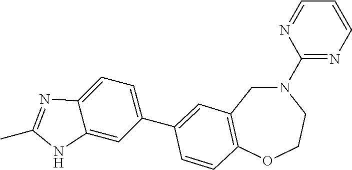 [SMILES: Cc1nc2ccc(-c3ccc4c(c3)CN(c3ncccn3)CCO4)cc2[nH]1] | 7-(2-methyl-1H-benzimidazol-6- yl)-4-pyridin-2-yl-2,3,4,5- tetrahydro-1,4-benzoxazepine |
| 107 | 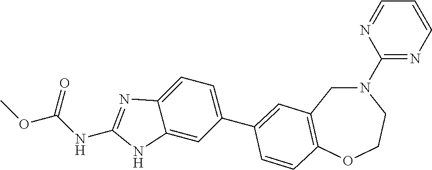 [SMILES: COC(=O)Nc1nc2ccc(-c3ccc4c(c3)CN(c3ncccn3)CCO4)cc2[nH]1] | methyl [6-(4-pyrimidin-4-yl- 2,3,4,5-tetrahydro-1,4- benzoxazepin-7-yl)-1H- benzimidazol-2-yl]carbamate |
| 108 | 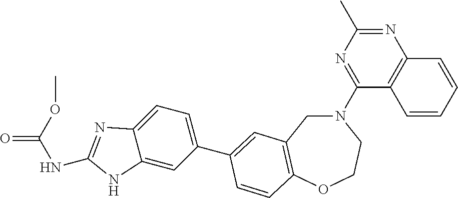 [SMILES: COC(=O)Nc1nc2ccc(-c3ccc4c(c3)CN(c3nc(C)nc5ccccc35)CCO4)cc2[nH]1] | methyl {6-[4-(2- methylquinazolin-4-yl)-2,3,4,5- tetrahydro-1,4-benzoxazepin-7- yl]-1H-benzimidazol-2- yl}carbamate |
| 109 | 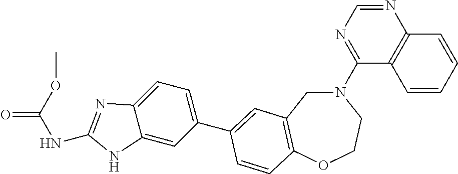 [SMILES: COC(=O)Nc1nc2ccc(-c3ccc4c(c3)CN(c3ncnc5ccccc35)CCO4)cc2[nH]1] | methyl [6-(4-quinazolin-4-yl- 2,3,4,5-tetrahydro-1,4- benzoxazepin-7-yl)-1H- benzimidazol-2-yl]carbamate |
| 110 | 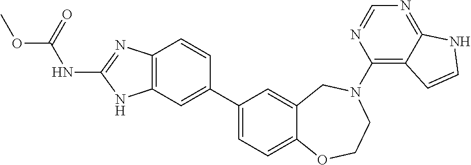 [SMILES: COC(=O)Nc1nc2ccc(-c3ccc4c(c3)CN(c3ncnc5[nH]ccc35)CCO4)cc2[nH]1] | methyl {6-[4-(7H-pyrrolo[2,3- d]pyrimidin-4-yl)-2,3,4,5- tetrahydro-1,4-benzoxazepin-7- yl]-1H-benzimidazol-2- yl}carbamate |
| 111 | 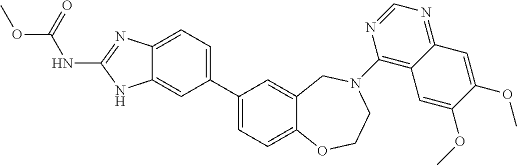 | methyl (6-{4-[6,7- bis(methyloxy)quinazolin-4-yl]- 2,3,4,5-tetrahydro-1,4- benzoxazepin-7-yl}-1H- benzimidazol-2-yl)carbamate |
| 112 | 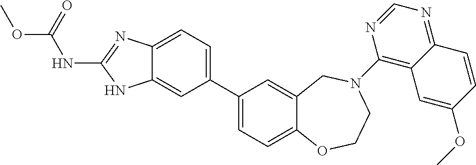 [SMILES: COC(=O)Nc1nc2ccc(-c3ccc4c(c3)CN(c3ncnc5ccc(OC)cc35)CCO4)cc2[nH]1] | methyl (6-{4-[6- (methyloxy)quinazolin-4-yl]- 2,3,4,5-tetrahydro-1,4- benzoxazepin-7-yl}-1H- benzimidazol-2-yl)carbamate |
| 113 | 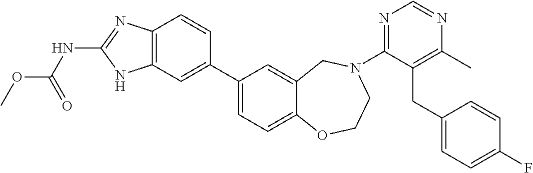 [SMILES: COC(=O)Nc1nc2ccc(-c3ccc4c(c3)CN(c3ncnc(C)c3Cc3ccc(F)cc3)CCO4)cc2[nH]1] | methyl [6-(4-{5-[(4- fluorophenyl)methyl]-6- methylpyrimidin-4-yl}-2,3,4,5- tetrahydro-1,4-benzoxazepin-7- yl)-1H-benzimidazol-2- yl]carbamate |
| 114 | 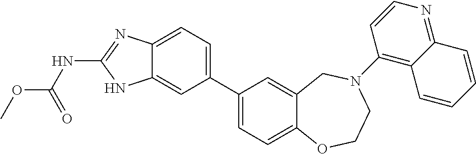 [SMILES: COC(=O)Nc1nc2ccc(-c3ccc4c(c3)CN(c3ccnc5ccccc35)CCO4)cc2[nH]1] | methyl [6-(4-quinolin-4-yl- 2,3,4,5-tetrahydro-1,4- benzoxazepin-7-yl)-1H- benzimidazol-2-yl]carbamate |
| 115 | 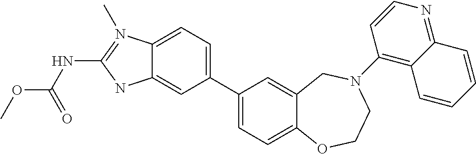 | methyl [1-methyl-5-(4-quinolin- 4-yl-2,3,4,5-tetrahydro-1,4- benzoxazepin-7-yl)-1H- benzimidazol-2-yl]carbamate |
| 116 | 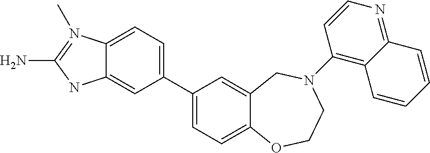 | 1-methyl-5-(4-quinolin-4-yl- 2,3,4,5-tetrahydro-1,4- benzoxazepin-7-yl)-1H- benzimidazol-2-amine |
| 117 | 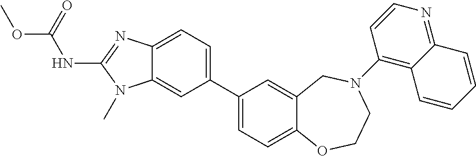 [SMILES: COC(=O)Nc1nc2ccc(-c3ccc4c(c3)CN(c3ccnc5ccccc35)CCO4)cc2n1C] | methyl [1-methyl-6-(4-quinolin- 4-yl-2,3,4,5-tetrahydro-1,4- benzoxazepin-7-yl)-1H- benzimidazol-2-yl]carbamate |
| 118 | 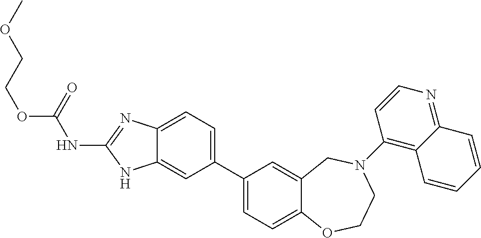 [SMILES: COCCOC(=O)Nc1nc2ccc(-c3ccc4c(c3)CN(c3ccnc5ccccc35)CCO4)cc2[nH]1] | 2-(methyloxy)ethyl [6-(4- quinolin-4-yl-2,3,4,5-tetrahydro- 1,4-benzoxazepin-7-yl)-1H- benzimidazol-2-yl]carbamate |
| 119 | 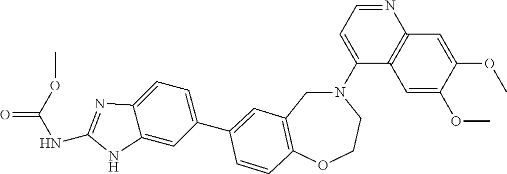 [SMILES: COC(=O)Nc1nc2ccc(-c3ccc4c(c3)CN(c3ccnc5cc(OC)c(OC)cc35)CCO4)cc2[nH]1] | methyl (6-{4-[6,7- bis(methyloxy)quinolin-4-yl]- 2,3,4,5-tetrahydro-1,4- benzoxazepin-7-yl}-1H- benzimidazol-2-yl)carbamate |
| 120 | 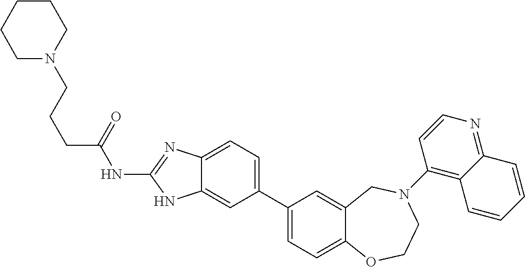 [SMILES: O=C(CCCN1CCCCC1)Nc1nc2ccc(-c3ccc4c(c3)CN(c3ccnc5ccccc35)CCO4)cc2[nH]1] | 4-piperidin-1-yl-N-[6-(4- quinolin-4-yl-2,3,4,5-tetrahydro- 1,4-benzoxazepin-7-yl)-1H- benzimidazol-2-yl]butanamide |
| 121 | 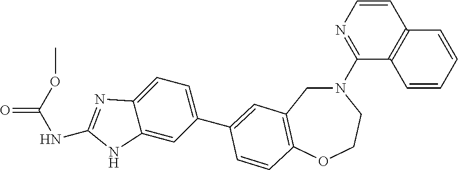 [SMILES: COC(=O)Nc1nc2ccc(-c3ccc4c(c3)CN(c3nccc5ccccc35)CCO4)cc2[nH]1] | methyl [6-(4-isoquinolin-1-yl- 2,3,4,5-tetrahydro-1,4- benzoxazepin-7-yl)-1H- benzimidazol-2-yl]carbamate |
| 122 | 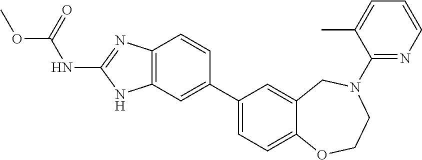 [SMILES: COC(=O)Nc1nc2ccc(-c3ccc4c(c3)CN(c3ncccc3C)CCO4)cc2[nH]1] | methyl {6-[4-(3-methylpyridin- 2-yl)-2,3,4,5-tetrahydro-1,4- benzoxazepin-7-yl]-1H- benzimidazol-2-yl} carbamate |
| 123 | 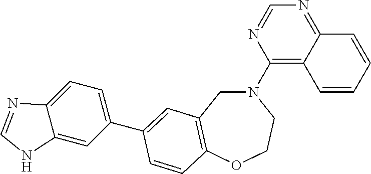 [SMILES: c1ccc2c(N3CCOc4ccc(-c5ccc6nc[nH]c6c5)cc4C3)ncnc2c1] | 7-(1H-benzimidazol-6-yl)-4- quinazolin-4-yl-2,3,4,5- tetrahydro-1,4-benzoxazepine |
| 124 | 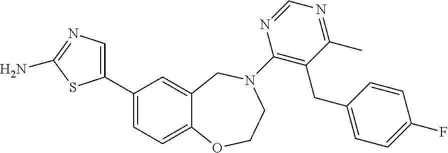 [SMILES: Cc1ncnc(N2CCOc3ccc(-c4cnc(N)s4)cc3C2)c1Cc1ccc(F)cc1] | 5-(4-{5-[(4- fluorophenyl)methyl]-6- methylpyrimidin-4-yl}-2,3,4,5- tetrahydro-1,4-benzoxazepin-7- yl)-1,3-thiazol-2-amine |
| 125 | 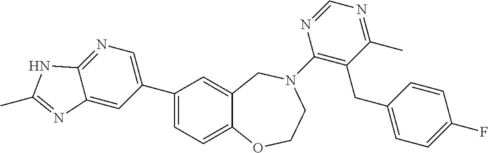 [SMILES: Cc1nc2cc(-c3ccc4c(c3)CN(c3ncnc(C)c3Cc3ccc(F)cc3)CCO4)cnc2[nH]1] | 4-{5-[(4-fluorophenyl)methyl]- 6-methylpyrimidin-4-yl}-7-(2- methyl-3H-imidazo[4,5- b]pyridin-6-yl)-2,3,4,5- tetrahydro-1,4-benzoxazepine |
| 126 | 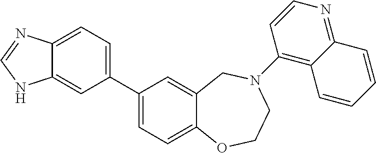 [SMILES: c1ccc2c(N3CCOc4ccc(-c5ccc6nc[nH]c6c5)cc4C3)ccnc2c1] | 7-(1H-benzimidazol-6-yl)-4- quinolin-4-yl-2,3,4,5-tetrahydro- 1,4-benzoxazepine |
| 127 | 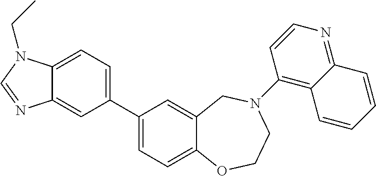 [SMILES: CCn1cnc2cc(-c3ccc4c(c3)CN(c3ccnc5ccccc35)CCO4)ccc21] | 7-(1-ethyl-1H-benzimidazol-5- yl)-4-quinolin-4-yl-2,3,4,5- tetrahydro-1,4-benzoxazepine |
| 128 | 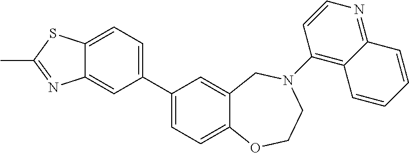 [SMILES: Cc1nc2cc(-c3ccc4c(c3)CN(c3ccnc5ccccc35)CCO4)ccc2s1] | 7-(2-methyl-1,3-benzothiazol-5- yl)-4-quinolin-4-yl-2,3,4,5- tetrahydro-1,4-benzoxazepine |
| 129 | 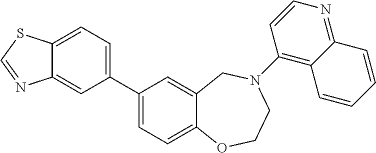 [SMILES: c1ccc2c(N3CCOc4ccc(-c5ccc6scnc6c5)cc4C3)ccnc2c1] | 7-(1,3-benzothiazol-5-yl)-4- quinolin-4-yl-2,3,4,5-tetrahydro- 1,4-benzoxazepine |
| 130 | 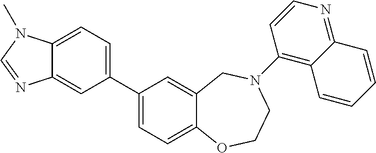 [SMILES: Cn1cnc2cc(-c3ccc4c(c3)CN(c3ccnc5ccccc35)CCO4)ccc21] | 7-(1-methyl-1H-benzimidazol-5- yl)-4-quinolin-4-yl-2,3,4,5- tetrahydro-1,4-benzoxazepine |
| 131 | 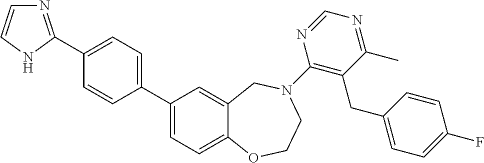 [SMILES: Cc1ncnc(N2CCOc3ccc(-c4ccc(-c5ncc[nH]5)cc4)cc3C2)c1Cc1ccc(F)cc1] | 4-{5-[(4-fluorophenyl)methyl]- 6-methylpyrimidin-4-yl}-7-[4- (1H-imidazol-2-yl)phenyl]- 2,3,4,5-tetrahydro-1,4- benzoxazepine |
| 132 | 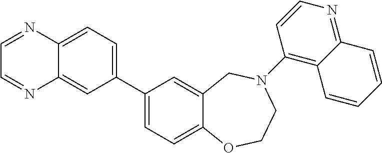 [SMILES: c1ccc2c(N3CCOc4ccc(-c5ccc6nccnc6c5)cc4C3)ccnc2c1] | 4-quinolin-4-yl-7-quinoxalin-6- yl-2,3,4,5-tetrahydro-1,4- benzoxazepine |
| 133 | 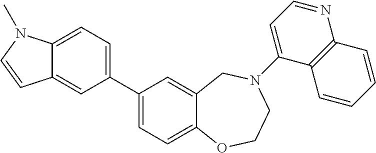 [SMILES: Cn1ccc2cc(-c3ccc4c(c3)CN(c3ccnc5ccccc35)CCO4)ccc21] | 7-(1-methyl-1H-indol-5-yl)-4- quinolin-4-yl-2,3,4,5-tetrahydro- 1,4-benzoxazepine |
| 134 | 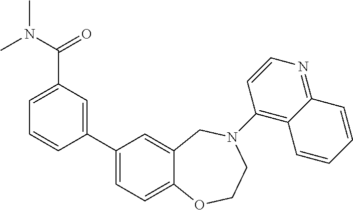 [SMILES: CN(C)C(=O)c1cccc(-c2ccc3c(c2)CN(c2ccnc4ccccc24)CCO3)c1] | N,N-dimethyl-3-(4-quinolin-4- yl-2,3,4,5-tetrahydro-1,4- benzoxazepin-7-yl)benzamide |
| 135 | 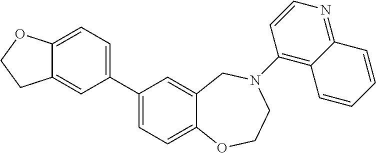 [SMILES: c1ccc2c(N3CCOc4ccc(-c5ccc6c(c5)CCO6)cc4C3)ccnc2c1] | 7-(2,3-dihydro-1-benzofuran-5- yl)-4-quinolin-4-yl-2,3,4,5- tetrahydro-1,4-benzoxazepine |
| 136 | 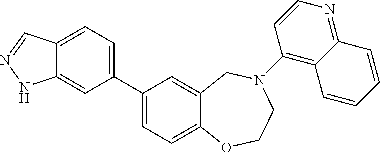 [SMILES: c1ccc2c(N3CCOc4ccc(-c5ccc6cn[nH]c6c5)cc4C3)ccnc2c1] | 7-(1H-indazol-6-yl)-4-quinolin- 4-yl-2,3,4,5-tetrahydro-1,4- benzoxazepine |
| 137 | 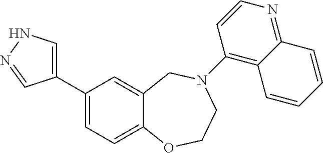 [SMILES: c1ccc2c(N3CCOc4ccc(-c5cn[nH]c5)cc4C3)ccnc2c1] | 7-(1H-pyrazol-4-yl)-4-quinolin- 4-yl-2,3,4,5-tetrahydro-1,4- benzoxazepine |
| 138 | 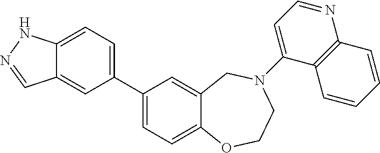 [SMILES: c1ccc2c(N3CCOc4ccc(-c5ccc6[nH]ncc6c5)cc4C3)ccnc2c1] | 7-(1H-indazol-5-yl)-4-quinolin- 4-yl-2,3,4,5-tetrahydro-1,4- benzoxazepine |
| 139 | 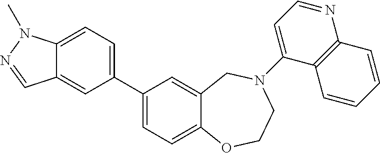 [SMILES: Cn1ncc2cc(-c3ccc4c(c3)CN(c3ccnc5ccccc35)CCO4)ccc21] | 7-(1-methyl-1H-indazol-5-yl)-4- quinolin-4-yl-2,3,4,5-tetrahydro- 1,4-benzoxazepine |
| 140 | 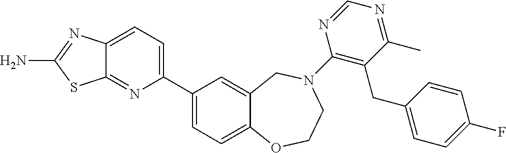 [SMILES: Cc1ncnc(N2CCOc3ccc(-c4ccc5nc(N)sc5n4)cc3C2)c1Cc1ccc(F)cc1] | 5-(4-{5-[(4- fluorophenyl)methyl]-6- methylpyrimidin-4-yl}-2,3,4,5- tetrahydro-1,4-benzoxazepin-7- yl)[1,3]thiazolo[5,4-b]pyridin-2- amine |
| 141 | 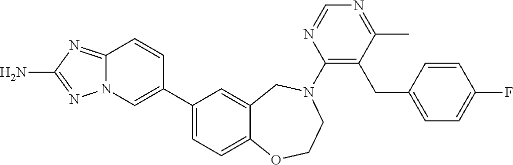 [SMILES: Cc1ncnc(N2CCOc3ccc(-c4ccc5nc(N)nn5c4)cc3C2)c1Cc1ccc(F)cc1] | 6-(4-{5-[(4- fluorophenyl)methyl]-6- methylpyrimidin-4-yl}-2,3,4,5- tetrahydro-1,4-benzoxazepin-7- yl)[1,2,4]triazolo[1,5-a]pyridin- 2-amine |
| 142 | 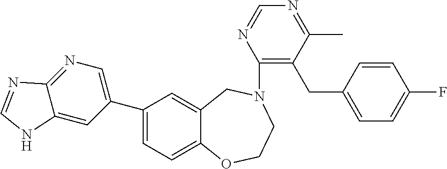 [SMILES: Cc1ncnc(N2CCOc3ccc(-c4cnc5nc[nH]c5c4)cc3C2)c1Cc1ccc(F)cc1] | 4-{5-[(4-fluorophenyl)methyl]- 6-methylpyrimidin-4-yl}-7-(1H- imidazo[4,5-b]pyridin-6-yl)- 2,3,4,5-tetrahydro-1,4- benzoxazepine |
| 143 | 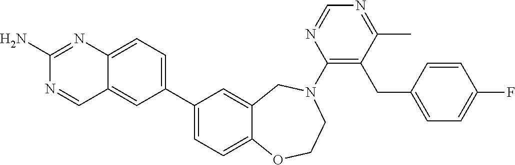 [SMILES: Cc1ncnc(N2CCOc3ccc(-c4ccc5nc(N)ncc5c4)cc3C2)c1Cc1ccc(F)cc1] | 6-(4-{5-[(4- fluorophenyl)methyl]-6- methylpyrimidin-4-yl}-2,3,4,5- tetrahydro-1,4-benzoxazepin-7- yl)quinazolin-2-amine |
| 144 | 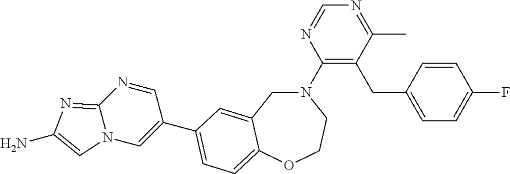 [SMILES: Cc1ncnc(N2CCOc3ccc(-c4cnc5nc(N)cn5c4)cc3C2)c1Cc1ccc(F)cc1] | 6-(4-{5-[(4- fluorophenyl)methyl]-6- methylpyrimidin-4-yl}-2,3,4,5- tetrahydro-1,4-benzoxazepin-7- yl)imidazo[1,2-a]pyrimidin-2- amine |
| 145 | 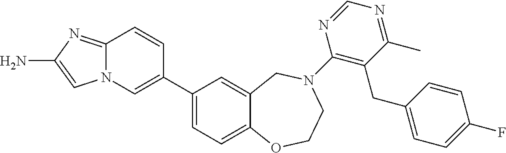 [SMILES: Cc1ncnc(N2CCOc3ccc(-c4ccc5nc(N)cn5c4)cc3C2)c1Cc1ccc(F)cc1] | 7-(4-{5-[(4- fluorophenyl)methyl]-6- methylpyrimidin-4-yl}-2,3,4,5- tetrahydro-1,4-benzoxazepin-7- yl)imidazo[1,2-a]pyridin-2- amine |
| 146 | 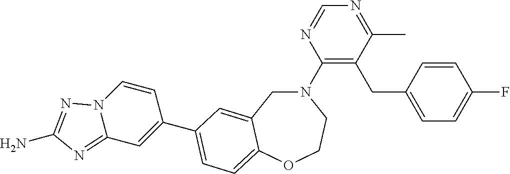 [SMILES: Cc1ncnc(N2CCOc3ccc(-c4ccn5nc(N)nc5c4)cc3C2)c1Cc1ccc(F)cc1] | 6-(4-{5-[(4- fluorophenyl)methyl]-6- methylpyrimidin-4-yl}-2,3,4,5- tetrahydro-1,4-benzoxazepin-7- yl)[1,2,4]triazolo[1,5-a]pyridin- 2-amine |
| 147 | 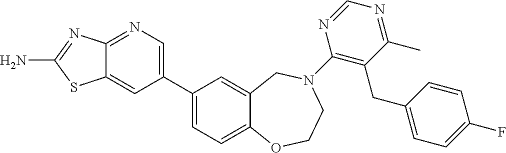 [SMILES: Cc1ncnc(N2CCOc3ccc(-c4cnc5nc(N)sc5c4)cc3C2)c1Cc1ccc(F)cc1] | 5-(4-{5-[(4- fluorophenyl)methyl]-6- methylpyrimidin-4-yl}-2,3,4,5- tetrahydro-1,4-benzoxazepin-7- yl)[1,3]thiazolo[4,5-b]pyridin-2- amine |
| 148 | 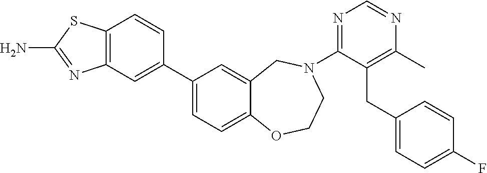 [SMILES: Cc1ncnc(N2CCOc3ccc(-c4ccc5sc(N)nc5c4)cc3C2)c1Cc1ccc(F)cc1] | 5-(4-{5-[(4- fluorophenyl)methyl]-6- methylpyrimidin-4-yl}-2,3,4,5- tetrahydro-1,4-benzoxazepin-7- yl)-1,3-benzothiazol-2-amine |
| 149 | 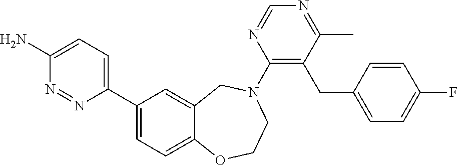 [SMILES: Cc1ncnc(N2CCOc3ccc(-c4ccc(N)nn4)cc3C2)c1Cc1ccc(F)cc1] | 6-(4-{5-[(4- fluorophenyl)methyl]-6- methylpyrimidin-4-yl}-2,3,4,5- tetrahydro-1,4-benzoxazepin-7- yl)pyridazin-3-amine |
| 150 | 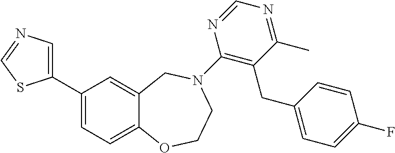 [SMILES: Cc1ncnc(N2CCOc3ccc(-c4cncs4)cc3C2)c1Cc1ccc(F)cc1] | 4-{5-[(4-fluorophenyl)methyl]- 6-methylpyrimidin-4-yl}-7-(1,3- thiazol-5-yl)-2,3,4,5-tetrahydro- 1,4-benzoxazepine |
| 151 | 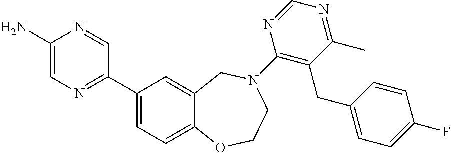 [SMILES: Cc1ncnc(N2CCOc3ccc(-c4cnc(N)cn4)cc3C2)c1Cc1ccc(F)cc1] | 5-(4-{5-[(4- fluorophenyl)methyl]-6- methylpyrimidin-4-yl}-2,3,4,5- tetrahydro-1,4-benzoxazepin-7- yl)pyrazin-2-amine |
| 152 | 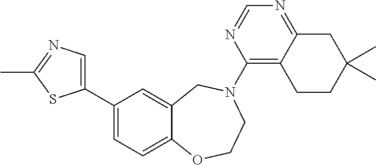 | 4-(7,7-dimethyl-5,6,7,8- tetrahydroquinazolin-4-yl)-7-(2- methyl-1,3-thiazol-5-yl)-2,3,4,5- tetrahydro-1,4-benzoxazepine |
| 153 | 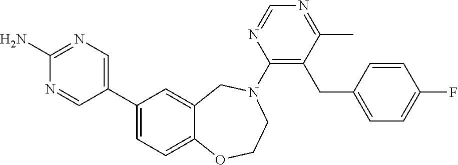 [SMILES: Cc1ncnc(N2CCOc3ccc(-c4cnc(N)nc4)cc3C2)c1Cc1ccc(F)cc1] | 5-(4-{5-[(4- fluorophenyl)methyl]-6- methylpyrimidin-4-yl}-2,3,4,5- tetrahydro-1,4-benzoxazepin-7- yl)pyrimidin-2-amine |
| 154 | 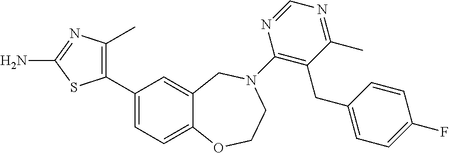 [SMILES: Cc1nc(N)sc1-c1ccc2c(c1)CN(c1ncnc(C)c1Cc1ccc(F)cc1)CCO2] | 5-(4-{5-[(4- fluorophenyl)methyl]-6- methylpyrimidin-4-yl}-2,3,4,5- tetrahydro-1,4-benzoxazepin-7- yl)-4-methyl-1,3-thiazol-2-amine |
| 155 | 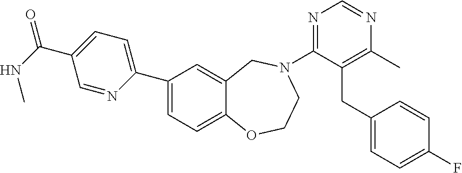 [SMILES: CNC(=O)c1ccc(-c2ccc3c(c2)CN(c2ncnc(C)c2Cc2ccc(F)cc2)CCO3)nc1] | 6-(4-{5-[(4- fluorophenyl)methyl]-6- methylpyrimidin-4-yl}-2,3,4,5- tetrahydro-1,4-benzoxazepin-7- yl)-N-methylpyridine-3- carboxamide |
| 156 | 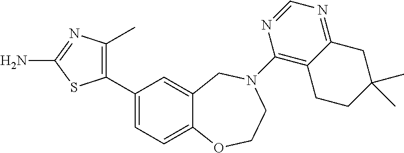 [SMILES: Cc1nc(N)sc1-c1ccc2c(c1)CN(c1ncnc3c1CCC(C)(C)C3)CCO2] | 5-[4-(7,7-dimethyl-5,6,7,8- tetrahydroquinazolin-4-yl)- 2,3,4,5-tetrahydro-1,4- benzoxazepin-7-yl]-4-methyl- 1,3-thiazol-2-amine |
| 157 | 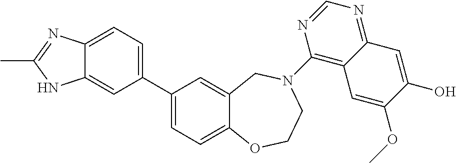 [SMILES: COc1cc2c(N3CCOc4ccc(-c5ccc6nc(C)[nH]c6c5)cc4C3)ncnc2cc1O] | 4-[7-(2-methyl-1H- benzimidazol-6-yl)-2,3-dihydro- 1,4-benzoxazepin-4(5H)-yl]-6- (methyloxy)quinazolin-7-ol |
| 158 | 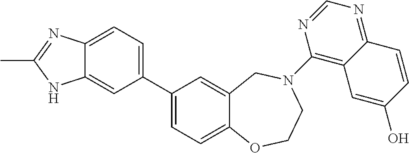 [SMILES: Cc1nc2ccc(-c3ccc4c(c3)CN(c3ncnc5ccc(O)cc35)CCO4)cc2[nH]1] | 4-[7-(2-methyl-1H- benzimidazol-6-yl)-2,3-dihydro- 1,4-benzoxazepin-4(5H)- yl]quinazolin-6-ol |
| 159 | 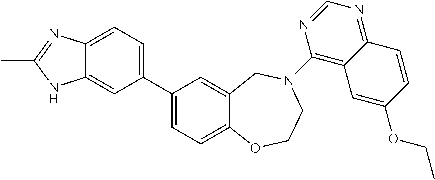 [SMILES: CCOc1ccc2ncnc(N3CCOc4ccc(-c5ccc6nc(C)[nH]c6c5)cc4C3)c2c1] | 4-[6-(ethyloxy)quinazolin-4-yl]- 7-(2-methyl-1H-benzimidazol-6- yl)-2,3,4,5-tetrahydro-1,4- benzoxazepine |
| 160 | 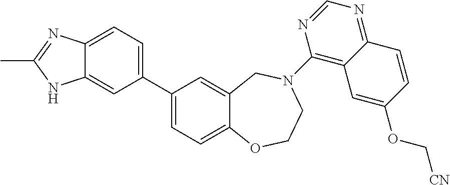 [SMILES: Cc1nc2ccc(-c3ccc4c(c3)CN(c3ncnc5ccc(OCC#N)cc35)CCO4)cc2[nH]1] | ({4-[7-(2- methyl-1H- benzimidazol-6-yl)-2,3-dihydro- 1,4-benzoxazepin-4(5H)- yl]quinazolin-6- yl}oxy)acetonitrile |
| 161 | 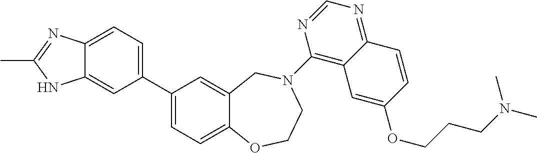 | N,N-dimethyl-3-({4-[7-(2- methyl-1H-benzimidazol-6-yl)- 2,3-dihydro-1,4-benzoxazepin- 4(5H)-yl]quinazolin-6- yl}oxy)propan-1-amine |
| 162 | 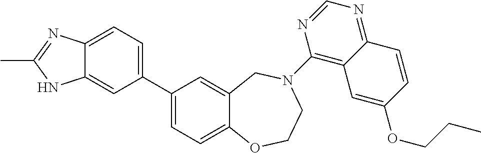 [SMILES: CCCOc1ccc2ncnc(N3CCOc4ccc(-c5ccc6nc(C)[nH]c6c5)cc4C3)c2c1] | 7-(2-methyl-1H-benzimidazol-6- yl)-4-[6-(propyloxy)quinazolin- 4-yl]-2,3,4,5-tetrahydro-1,4- benzoxazepine |
| 163 | 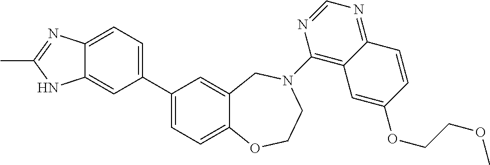 | 7-(2-methyl-1H-benzimidazol-6- yl)-4-(6-([2- (methyloxy)ethyl]oxy}quinazolin- 4-yl)-2,3,4,5-tetrahydro-1,4- benzoxazepine |
| 164 | 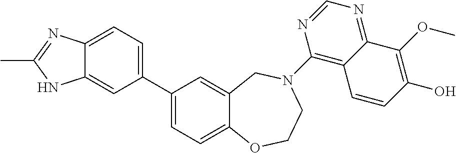 | 4-[7-(2-methyl-1H- benzimidazol-6-yl)-2,3-dihydro- 1,4-benzoxazepin-4(5H)-yl]-8- (methyloxy)quinazolin-7-ol |
| 165 | 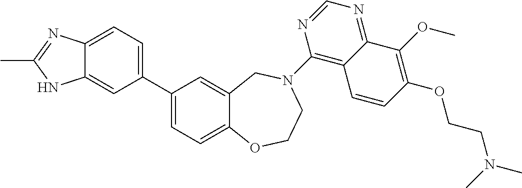 | N,N-dimethyl-2-({4-[7-(2- methyl-1H-benzimidazol-6-yl)- 2,3-dihydro-1,4-benzoxazepin- 4(5H)-yl]-8- (methyloxy)quinazolin-7- yl}oxy)ethanamine |
| 166 | 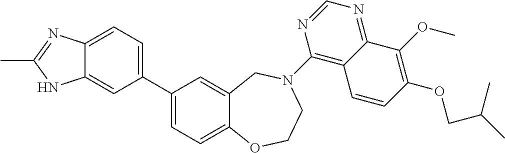 [SMILES: COc1c(OCC(C)C)ccc2c(N3CCOc4ccc(-c5ccc6nc(C)[nH]c6c5)cc4C3)ncnc12] | 7-(2-methyl-1H-benzimidazol-6- yl)-4-{8-(methyloxy)-7-[(2- methylpropyl)oxy]quinazolin-4- yl}-2,3,4,5-tetrahydro-1,4- benzoxazepine |
| 167 | 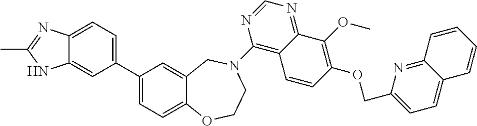 [SMILES: COc1c(OCc2ccc3ccccc3n2)ccc2c(N3CCOc4ccc(-c5ccc6nc(C)[nH]c6c5)cc4C3)ncnc12] | 7-(2-methyl-1H-benzimidazol-6- yl)-4-{8-(methyloxy)-7- [(quinolin-2- ylmethyl)oxy}quinazolin-4-yl}- 2,3,4,5-tetrahydro-1,4- benzoxazepine |
| 168 | 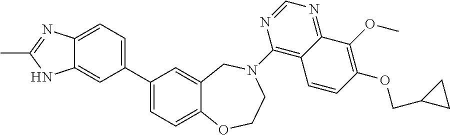 [SMILES: COc1c(OCC2CC2)ccc2c(N3CCOc4ccc(-c5ccc6nc(C)[nH]c6c5)cc4C3)ncnc12] | 4-{7-[(cyclopropylmethyl)oxy]- 8-(methyloxy)quinazolin-4-yl}- 7-(2-methyl-1H-benzimidazol-6- yl)-2,3,4,5-tetrahydro-1,4- benzoxazepine |
| 169 | 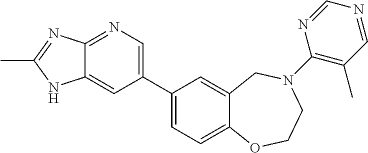 [SMILES: Cc1nc2ncc(-c3ccc4c(c3)CN(c3ncncc3C)CCO4)cc2[nH]1] | 7-(2-methyl-1H-imidazo[4,5- b]pyridin-6-yl)-4-(5- methylpyrimidin-4-yl)-2,3,4,5- tetrahydro-1,4-benzoxazepine |
| 170 | 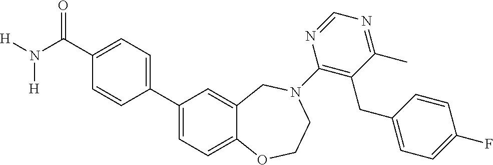 [SMILES: Cc1ncnc(N2CCOc3ccc(-c4ccc(C(N)=O)cc4)cc3C2)c1Cc1ccc(F)cc1] | 4-(4-{5-[(4- fluorophenyl)methyl]-6- methylpyrimidin-4-yl}-2,3,4,5- tetrahydro-1,4-benzoxazepin-7- yl)benzamide |
| 171 | 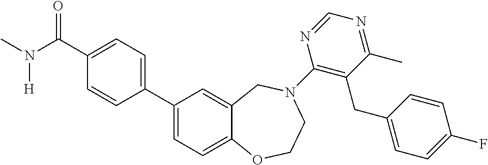 [SMILES: CNC(=O)c1ccc(-c2ccc3c(c2)CN(c2ncnc(C)c2Cc2ccc(F)cc2)CCO3)cc1] | 4-(4-{5-[(4- fluorophenyl)methyl]-6- methylpyrimidin-4-yl}-2,3,4,5- tetrahydro-1,4-benzoxazepin-7- yl)-N-methylbenzamide |
| 172 | 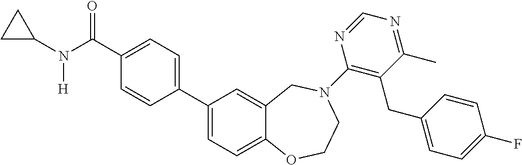 [SMILES: Cc1ncnc(N2CCOc3ccc(-c4ccc(C(=O)NC5CC5)cc4)cc3C2)c1Cc1ccc(F)cc1] | N-cyclopropyl-4-(4-{5-[(4- fluorophenyl)methyl]-6- methylpyrimidin-4-yl}-2,3,4,5- tetrahydro-1,4-benzoxazepin-7- yl)benzamide |
| 173 | 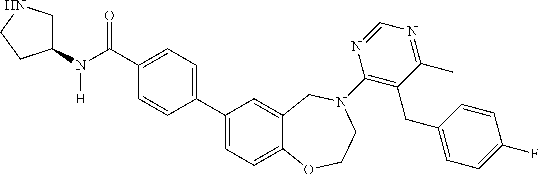 [SMILES: Cc1ncnc(N2CCOc3ccc(-c4ccc(C(=O)N[C@H]5CCNC5)cc4)cc3C2)c1Cc1ccc(F)cc1] | 4-(4-{5-[(4- fluorophenyl)methyl]-6- methylpyrimidin-4-yl}-2,3,4,5- tetrahydro-1,4-benzoxazepin-7- yl)-N-[(3S)-pyrrolidin-3- yl]benzamide |
| 174 | 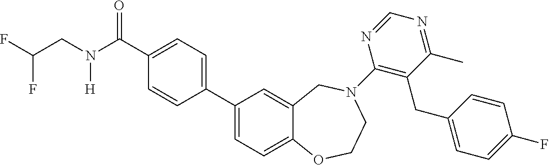 [SMILES: Cc1ncnc(N2CCOc3ccc(-c4ccc(C(=O)NCC(F)F)cc4)cc3C2)c1Cc1ccc(F)cc1] | N-(2,2-difluoroethyl)-4-(4-{5- [(4-fluorophenyl)methyl]-6- methylpyrimidin-4-yl}-2,3,4,5- tetrahydro-1,4-benzoxazepin-7- yl)benzamide |
| 175 | 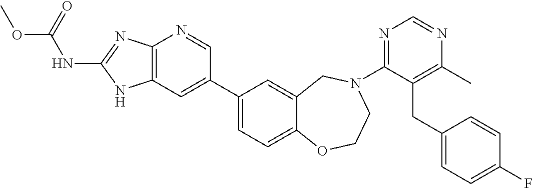 [SMILES: COC(=O)Nc1nc2ncc(-c3ccc4c(c3)CN(c3ncnc(C)c3Cc3ccc(F)cc3)CCO4)cc2[nH]1] | methyl [6-(4-{5-[(4- fluorophenyl)methyl]-6- methylpyrimidin-4-yl}-2,3,4,5- tetrahydro-1,4-benzoxazepin-7- yl)-1H-imidazo[4,5-b]pyridin-2- yl]carbamate |
| 176 | 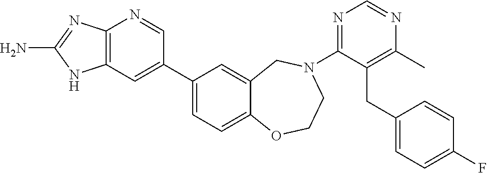 [SMILES: Cc1ncnc(N2CCOc3ccc(-c4cnc5nc(N)[nH]c5c4)cc3C2)c1Cc1ccc(F)cc1] | 6-(4-{5-[(4- fluorophenyl)methyl]-6- methylpyrimidin-4-yl}-2,3,4,5- tetrahydro-1,4-benzoxazepin-7- yl)-1H-imidazo[4,5-b]pyridin-2- amine |
| 177 | 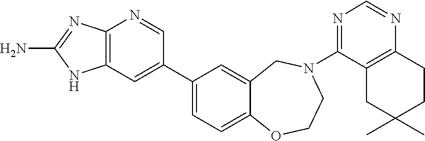 [SMILES: CC1(C)CCc2ncnc(N3CCOc4ccc(-c5cnc6nc(N)[nH]c6c5)cc4C3)c2C1] | 6-[4-(6,6-dimethyl-5,6,7,8- tetrahydroquinazolin-4-yl)- 2,3,4,5-tetrahydro-1,4- benzoxazepin-7-yl]-1H- imidazo[4,5-b]pyridin-2-amine |
| 178 | 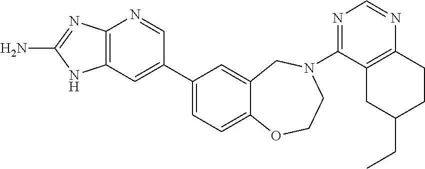 [SMILES: CCC1CCc2ncnc(N3CCOc4ccc(-c5cnc6nc(N)[nH]c6c5)cc4C3)c2C1] | 6-[4-(6-ethyl-5,6,7,8- tetrahydroquinazolin-4-yl)- 2,3,4,5-tetrahydro-1,4- benzoxazepin-7-yl]-1H- imidazo[4,5-b]pyridin-2-amine |
| 179 | 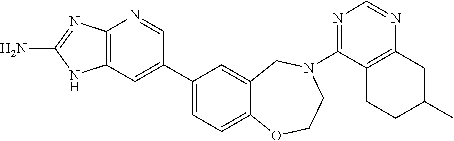 [SMILES: CC1CCc2c(ncnc2N2CCOc3ccc(-c4cnc5nc(N)[nH]c5c4)cc3C2)C1] | 6-[4-(7-methyl-5,6,7,8- tetrahydroquinazolin-4-yl)- 2,3,4,5-tetrahydro-1,4- benzoxazepin-7-yl]-1H- imidazo[4,5-b]pyridin-2-amine |
| 180 | 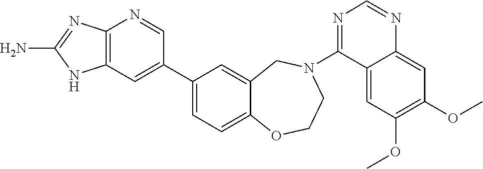 [SMILES: COc1cc2ncnc(N3CCOc4ccc(-c5cnc6nc(N)[nH]c6c5)cc4C3)c2cc1OC] | 6-{4-[6,7- bis(methyloxy)quinazolin-4-yl]- 2,3,4,5-tetrahydro-1,4- benzoxazepin-7-yl}-1H- imidazo[4,5-b]pyridin-2-amine |
| 181 | 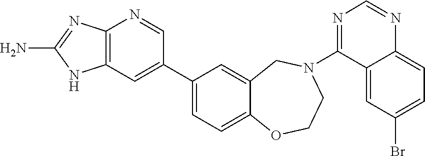 [SMILES: Nc1nc2ncc(-c3ccc4c(c3)CN(c3ncnc5ccc(Br)cc35)CCO4)cc2[nH]1] | 6-[4-(6-bromoquinazolin-4-yl)- 2,3,4,5-tetrahydro-1,4- benzoxazepin-7-yl]-1H- imidazo[4,5-b]pyridin-2-amine |
| 182 | 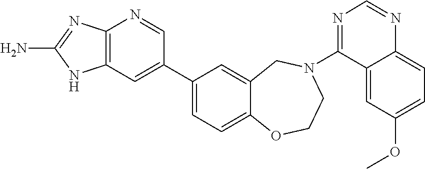 [SMILES: COc1ccc2ncnc(N3CCOc4ccc(-c5cnc6nc(N)[nH]c6c5)cc4C3)c2c1] | 6-{4-[6-(methyloxy)quinazolin- 4-yl]-2,3,4,5-tetrahydro-1,4- benzoxazepin-7-yl}-1H- imidazo[4,5-b]pyridin-2-amine |
| 183 | 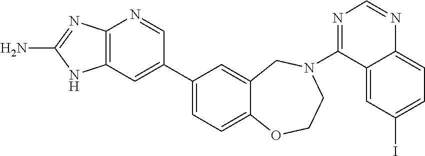 [SMILES: Nc1nc2ncc(-c3ccc4c(c3)CN(c3ncnc5ccc(I)cc35)CCO4)cc2[nH]1] | 6[4-(6-iodoquinazolin-4-yl)- 2,3,4,5-tetrahydro-1,4- benzoxazepin-7-yl]-1H- imidazo[4,5-b]pyridin-2-amine |
| 184 | 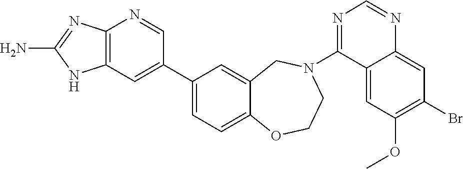 [SMILES: COc1cc2c(N3CCOc4ccc(-c5cnc6nc(N)[nH]c6c5)cc4C3)ncnc2cc1Br] | 6-{4-[7-bromo-6- (methyloxy)quinazolin-4-yl]- 2,3,4,5-tetrahydro-1,4- benzoxazepin-7-yl}-1H- imidazo[4,5-b]pyridin-2-amine |
| 185 | 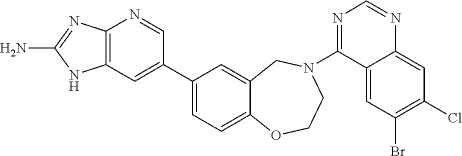 [SMILES: Nc1nc2ncc(-c3ccc4c(c3)CN(c3ncnc5cc(Cl)c(Br)cc35)CCO4)cc2[nH]1] | 6-[4-(6-bromo-7- chloroquinazolin-4-yl)-2,3,4,5- tetrahydro-1,4-benzoxazepin-7- yl]-1H-imidazo[4,5-b]pyridin-2- amine |
| 186 | 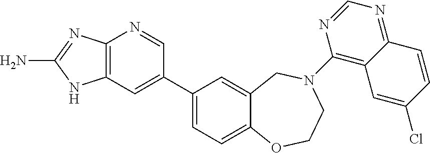 [SMILES: Nc1nc2ncc(-c3ccc4c(c3)CN(c3ncnc5ccc(Cl)cc35)CCO4)cc2[nH]1] | 6-[4-(6-chloroquinazolin-4-yl)- 2,3,4,5-tetrahydro-1,4- benzoxazepin-7-yl]-1H- imidazo[4,5-b]pyridin-2-amine |
| 187 | 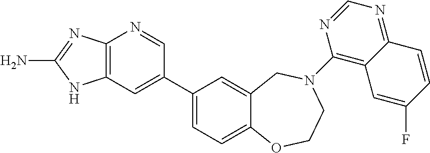 [SMILES: Nc1nc2ncc(-c3ccc4c(c3)CN(c3ncnc5ccc(F)cc35)CCO4)cc2[nH]1] | 6-[4-(6-fluoroquinazolin-4-yl)- 2,3,4,5-tetrahydro-1,4- benzoxazepin-7-yl]-1H- imidazo[4,5-b]pyridin-2-amine |
| 188 | 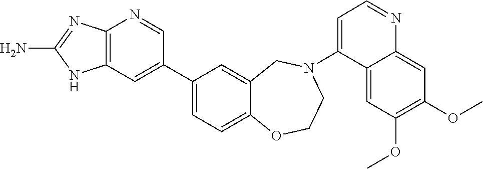 [SMILES: COc1cc2nccc(N3CCOc4ccc(-c5cnc6nc(N)[nH]c6c5)cc4C3)c2cc1OC] | 6-{4-[6,7- bis(methyloxy)quinolin-4-yl]- 2,3,4,5-tetrahydro-1,4- benzoxazepin-7-yl}-1H- imidazo[4,5-b]pyridin-2-amine |
| 189 | 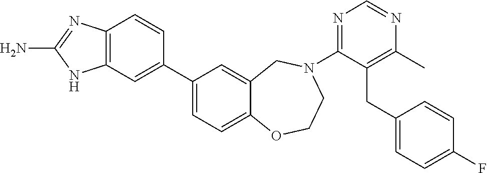 [SMILES: Cc1ncnc(N2CCOc3ccc(-c4ccc5nc(N)[nH]c5c4)cc3C2)c1Cc1ccc(F)cc1] | 6-(4-{5-[(4- fluorophenyl)methyl]-6- methylpyrimidin-4-yl}-2,3,4,5- tetrahydro-1,4-benzoxazepin-7- yl)-1H-benzimidazol-2-amine |
| 190 | 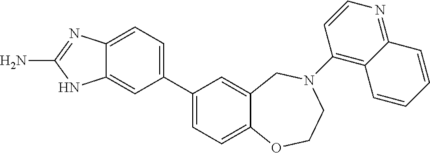 [SMILES: Nc1nc2ccc(-c3ccc4c(c3)CN(c3ccnc5ccccc35)CCO4)cc2[nH]1] | 6-(4-quinolin-4-yl-2,3,4,5- tetrahydro-1,4-benzoxazepin-7- yl)-1H-benzimidazol-2-amine |
| 191 | 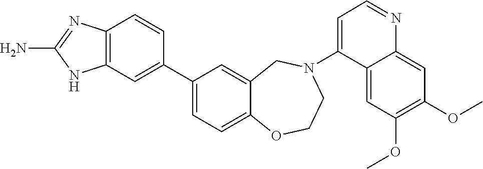 [SMILES: COc1cc2nccc(N3CCOc4ccc(-c5ccc6nc(N)[nH]c6c5)cc4C3)c2cc1OC] | 6-{4-[6,7- bis(methyloxy)quinolin-4-yl]- 2,3,4,5-tetrahydro-1,4- benzoxazepin-7-yl}-1H- benzimidazol-2-amine |
| 192 | 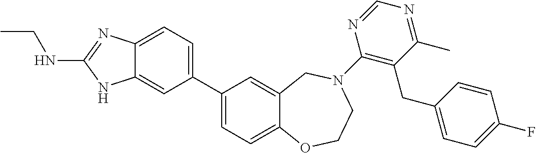 [SMILES: CCNc1nc2ccc(-c3ccc4c(c3)CN(c3ncnc(C)c3Cc3ccc(F)cc3)CCO4)cc2[nH]1] | N-ethyl-6-(4-{5-[(4- fluorophenyl)methyl]-6- methylpyrimidin-4-yl}-2,3,4,5- tetrahydro-1,4-benzoxazepin-7- yl)-1H-benzimidazol-2-amine |
| 193 | 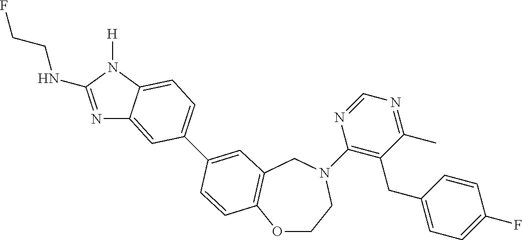 [SMILES: Cc1ncnc(N2CCOc3ccc(-c4ccc5[nH]c(NCCF)nc5c4)cc3C2)c1Cc1ccc(F)cc1] | N-(2-fluoroethyl)-5-(4-{5-[(4- fluorophenyl)methyl]-6- methylpyrimidin-4-yl}-2,3,4,5- tetrahydro-1,4-benzoxazepin-7- yl)-1H-benzimidazol-2-amine |
| 194 | 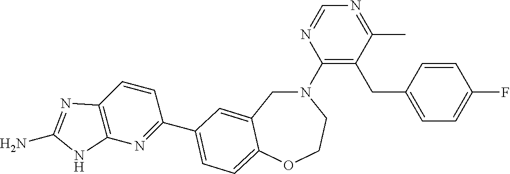 [SMILES: Cc1ncnc(N2CCOc3ccc(-c4ccc5nc(N)[nH]c5n4)cc3C2)c1Cc1ccc(F)cc1] | 5-(4-{5-[(4- fluorophenyl)methyl]-6- methylpyrimidin-4-yl)-2,3,4,5- tetrahydro-1,4-benzoxazepin-7- yl)-3H-imidazo[4,5-b]pyridin-2- amine |
| 195 | 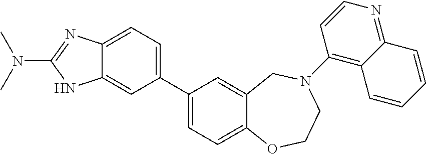 [SMILES: CN(C)c1nc2ccc(-c3ccc4c(c3)CN(c3ccnc5ccccc35)CCO4)cc2[nH]1] | N,N-dimethyl-6-(4-quinolin-4- yl-2,3,4,5-tetrahydro-1,4- benzoxazepin-7-yl)-1H- benzimidazol-2-amine |
| 196 | 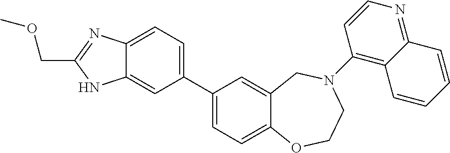 [SMILES: COCc1nc2ccc(-c3ccc4c(c3)CN(c3ccnc5ccccc35)CCO4)cc2[nH]1] | 7-{2-[(methyloxy)methyl]-1H- benzimidazol-6-yl}-4-quinolin- 4-yl-2,3,4,5-tetrahydro-1,4- benzoxazepine |
| 197 | 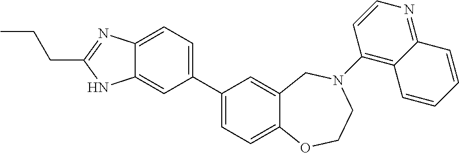 [SMILES: CCCc1nc2ccc(-c3ccc4c(c3)CN(c3ccnc5ccccc35)CCO4)cc2[nH]1] | 7-(2-propyl-1H-benzimidazol-6- yl)-4-quinolin-4-yl-2,3,4,5- tetrahydro-1,4-benzoxazepine |
| 198 | 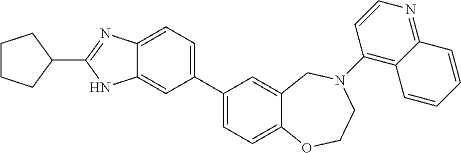 [SMILES: c1ccc2c(N3CCOc4ccc(-c5ccc6nc(C7CCCC7)[nH]c6c5)cc4C3)ccnc2c1] | 7-(2-cyclopentyl-1H- benzimidazol-6-yl)-4-quinolin- 4-yl-2,3,4,5-tetrahydro-1,4- benzoxazepine |
| 199 | 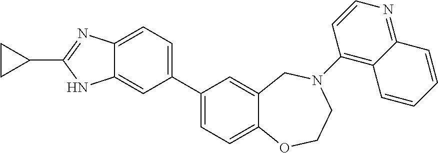 [SMILES: c1ccc2c(N3CCOc4ccc(-c5ccc6nc(C7CC7)[nH]c6c5)cc4C3)ccnc2c1] | 7-(2-cyclopropyl-1H- benzimidazol-6-yl)-4-quinolin- 4-yl-2,3,4,5-tetrahydro-1,4- benzoxazepine |
| 200 | 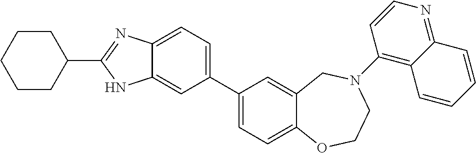 [SMILES: c1ccc2c(N3CCOc4ccc(-c5ccc6nc(C7CCCCC7)[nH]c6c5)cc4C3)ccnc2c1] | 7-(2-cyclohexyl-1H- benzimidazol-6-yl)-4-quinolin- 4-yl-2,3,4,5-tetrahydro-1,4- benzoxazepine |
| 201 | 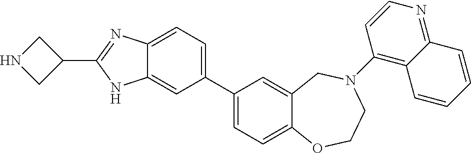 [SMILES: c1ccc2c(N3CCOc4ccc(-c5ccc6nc(C7CNC7)[nH]c6c5)cc4C3)ccnc2c1] | 7-(2-azetidin-3-yl-1H- benzimidazol-6-yl)-4-quinolin- 4-yl-2,3,4,5-tetrahydro-1,4- benzoxazepine |
| 202 | 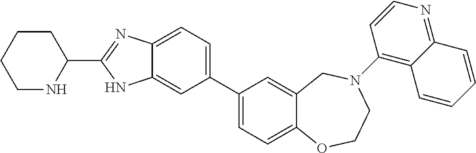 [SMILES: c1ccc2c(N3CCOc4ccc(-c5ccc6nc(C7CCCCN7)[nH]c6c5)cc4C3)ccnc2c1] | 7-(2-piperidin-2-yl-1H- benzimidazol-6-yl)-4-quinolin- 4-yl-2,3,4,5-tetrahydro-1,4- benzoxazepine |
| 203 | 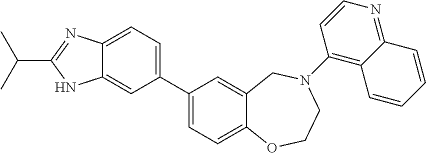 [SMILES: CC(C)c1nc2ccc(-c3ccc4c(c3)CN(c3ccnc5ccccc35)CCO4)cc2[nH]1] | 7-[2-(1-methylethyl)-1H- benzimidazol-6-yl]-4-quinolin- 4-yl-2,3,4,5-tetrahydro-1,4- benzoxazepine |
| 204 | 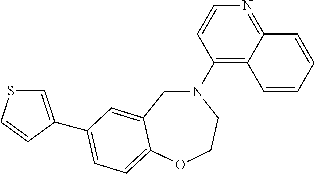 [SMILES: c1ccc2c(N3CCOc4ccc(-c5ccsc5)cc4C3)ccnc2c1] | 4-quinolin-4-yl-7-(3-thienyl)- 2,3,4,5-tetrahydro-1,4- benzoxazepine |
| 205 | 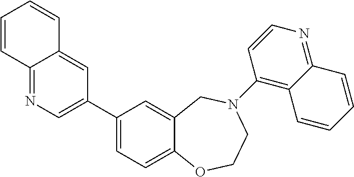 [SMILES: c1ccc2ncc(-c3ccc4c(c3)CN(c3ccnc5ccccc35)CCO4)cc2c1] | 7-quinolin-3-yl-4-quinolin-4-yl- 2,3,4,5-tetrahydro-1,4- benzoxazepine |
| 206 | 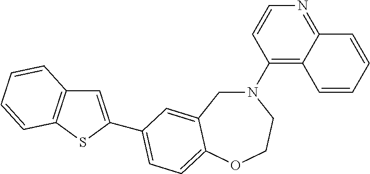 [SMILES: c1ccc2sc(-c3ccc4c(c3)CN(c3ccnc5ccccc35)CCO4)cc2c1] | 7-(1-benzothien-2-yl)-4- quinolin-4-yl-2,3,4,5-tetrahydro- 1,4-benzoxazepine |
| 207 | 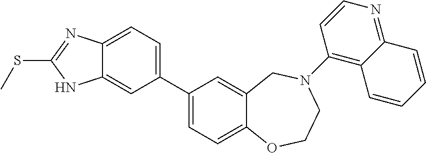 [SMILES: CSc1nc2ccc(-c3ccc4c(c3)CN(c3ccnc5ccccc35)CCO4)cc2[nH]1] | 7-[2-(methylthio)-1H- benzimidazol-6-yl]-4-quinolin- 4-yl-2,3,4,5-tetrahydro-1,4- benzoxazepine |
| 208 | 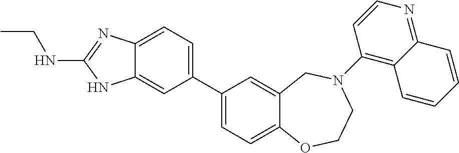 [SMILES: CCNc1nc2ccc(-c3ccc4c(c3)CN(c3ccnc5ccccc35)CCO4)cc2[nH]1] | N-ethyl-6-(4-quinolin-4-yl- 2,3,4,5-tetrahydro-1,4- benzoxazepin-7-yl)-1H- benzimidazol-2-amine |
| 209 | 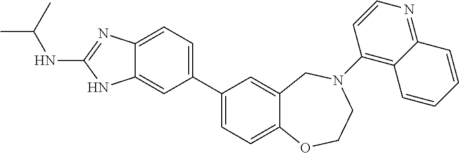 [SMILES: CC(C)Nc1nc2ccc(-c3ccc4c(c3)CN(c3ccnc5ccccc35)CCO4)cc2[nH]1] | N-(1-methylethyl)-6-(4-quinolin- 4-yl-2,3,4,5-tetrahydra-1,4- benzoxazepin-7-yl)-1H- benzimidazol-2-amine |
| 210 | 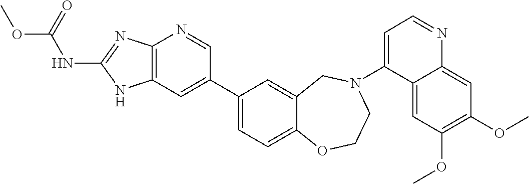 | methyl (6-{4-[6,7- bis(methyloxy)quinolin-4-yl]- 2,3,4,5-tetrahydro-1,4- benzoxazepin-7-yl}-1H- imidazo[4,5-b]pyridin-2- yl)carbamate |
| 211 | 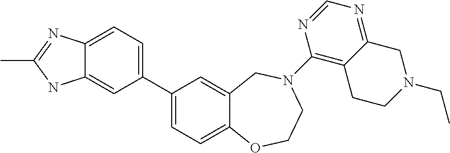 [SMILES: CCN1CCc2c(ncnc2N2CCOc3ccc(-c4ccc5nc(C)[nH]c5c4)cc3C2)C1] | 4-(7-ethyl-5,6,7,8- tetrahydropyrido[3,4- d]pyrimidin-4-yl)-7-(2-methyl- 1H-benzimidazol-6-yl)-2,3,4,5- tetrahydro-1,4-benzoxazepine |
| 212 | 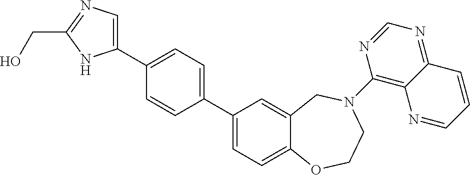 [SMILES: OCc1ncc(-c2ccc(-c3ccc4c(c3)CN(c3ncnc5cccnc35)CCO4)cc2)[nH]1] | {5-[4-(4-pyrido[3,2-d]pyrimidin- 4-yl-2,3,4,5-tetrahydro-1,4- benzoxazepin-7-yl)phenyl]-1H- imidazol-2-yl}methanol |
| 213 | 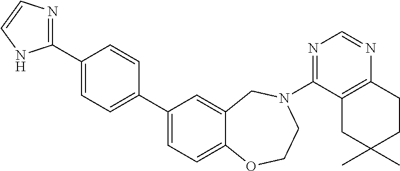 [SMILES: CC1(C)CCc2ncnc(N3CCOc4ccc(-c5ccc(-c6ncc[nH]6)cc5)cc4C3)c2C1] | 4-(6,6-dimethyl-5,6,7,8- tetrahydroquinazolin-4-yl)-7-[4- (1H-imidazol-2-yl)phenyl]- 2,3,4,5-tetrahydro-1,4- benzoxazepine |
| 214 | 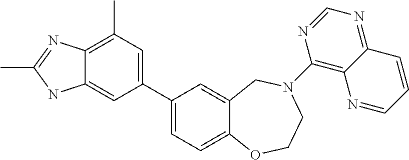 [SMILES: Cc1nc2c(C)cc(-c3ccc4c(c3)CN(c3ncnc5cccnc35)CCO4)cc2[nH]1] | 7-(2,4-dimethyl-1H- benzimidazol-6-yl)-4- pyrido[3,2-d]pyrimidin-4-yl- 2,3,4,5-tetrahydro-1,4- benzoxazepine |
| 215 | 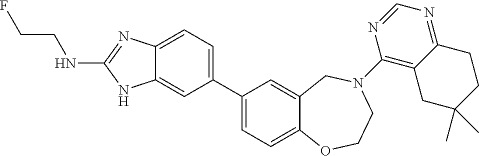 [SMILES: CC1(C)CCc2ncnc(N3CCOc4ccc(-c5ccc6nc(NCCF)[nH]c6c5)cc4C3)c2C1] | 6-[4-(6,6-dimethyl-5,6,7,8- tetrahydroquinazolin-4-yl)- 2,3,4,5-tetrahydro-1,4- benzoxazepin-7-yl]-N-(2- fluoroethyl)-1H-benzimidazol-2- amine |
| 216 | 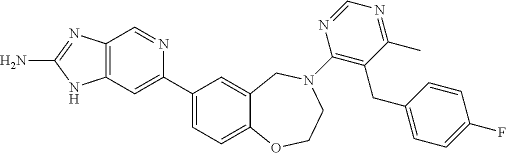 [SMILES: Cc1ncnc(N2CCOc3ccc(-c4cc5[nH]c(N)nc5cn4)cc3C2)c1Cc1ccc(F)cc1] | 6-(4-{5-[(4- fluorophenyl)methyl]-6- methylpyrimidin-4-yl}-2,3,4,5- tetrahydro-1,4-benzoxazepin-7- yl)-1H-imidazo[4,5-c]pyridin-2- amine |
| 217 | 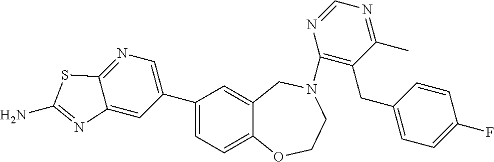 [SMILES: Cc1ncnc(N2CCOc3ccc(-c4cnc5sc(N)nc5c4)cc3C2)c1Cc1ccc(F)cc1] | 6-(4-{5-[(4- fluorophenyl)methyl]-6- methylpyrimidin-4-yl}-2,3,4,5- tetrahydro-1,4-benzoxazepin-7- yl)[1,3]thiazolo[5,4-b]pyridin-2- amine |
| 218 | 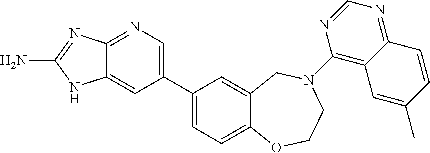 [SMILES: Cc1ccc2ncnc(N3CCOc4ccc(-c5cnc6nc(N)[nH]c6c5)cc4C3)c2c1] | 6-[4-(6-methylquinazolin-4-yl)- 2,3,4,5-tetrahydro-1,4- benzoxazepin-7-yl]-1H- imidazo[4,5-b]pyridin-2-amine |
| 219 | 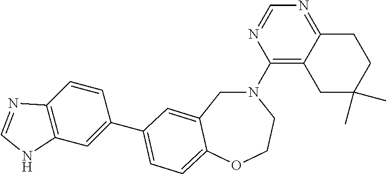 [SMILES: CC1(C)CCc2ncnc(N3CCOc4ccc(-c5ccc6nc[nH]c6c5)cc4C3)c2C1] | 7-(1H-benzimidazol-6-yl)-4- (6,6-dimethyl-5,6,7,8- tetrahydroquinazolin-4-yl)- 2,3,4,5-tetrahydro-1,4- benzoxazepine |
| 220 | 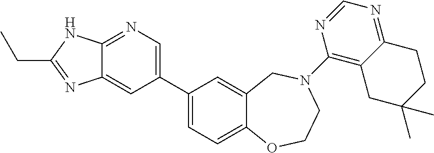 [SMILES: CCc1nc2cc(-c3ccc4c(c3)CN(c3ncnc5c3CC(C)(C)CC5)CCO4)cnc2[nH]1] | 4-(6,6-dimethyl-5,6,7,8- tetrahydroquinazolin-4-yl)-7-(2- ethyl-3H-imidazo[4,5-b]pyridin- 6-yl)-2,3,4,5-tetrahydro-1,4- benzoxazepine |
| 221 | 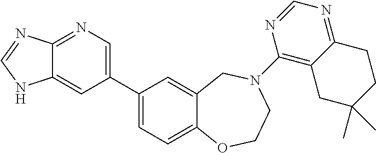 [SMILES: CC1(C)CCc2ncnc(N3CCOc4ccc(-c5cnc6nc[nH]c6c5)cc4C3)c2C1] | 4-(6,6-dimethyl-5,6,7,8- tetrahydroquinazolin-4-yl)-7- (1H-imidazo[4,5-b]pyridin-6- yl)-2,3,4,5-tetrahydro-1,4- benzoxazepine |
| 222 | 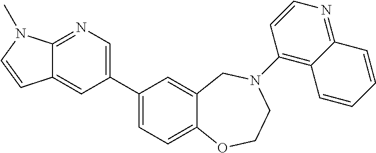 [SMILES: Cn1ccc2cc(-c3ccc4c(c3)CN(c3ccnc5ccccc35)CCO4)cnc21] | 7-(1-methyl-1H-pyrrolo[2,3- b]pyridin-5-yl)-4-quinolin-4-yl- 2,3,4,5-tetrahydro-1,4- benzoxazepine |
| 223 | 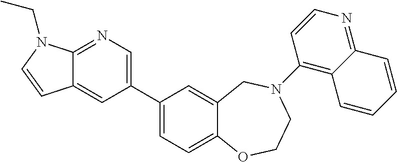 [SMILES: CCn1ccc2cc(-c3ccc4c(c3)CN(c3ccnc5ccccc35)CCO4)cnc21] | 7-(1-ethyl-1H-pyrrolo[2,3- b]pyridin-5-yl)-4-quinolin-4-yl- 2,3,4,5-tetrahydro-1,4- benzoxazepine |
| 224 | 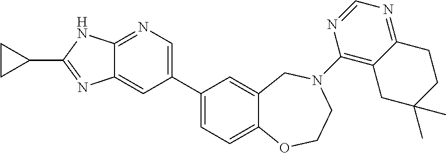 [SMILES: CC1(C)CCc2ncnc(N3CCOc4ccc(-c5cnc6[nH]c(C7CC7)nc6c5)cc4C3)c2C1] | 7-(2-cyclopropyl-3H- imidazo[4,5-b]pyridin-6-yl)-4- (6,6-dimethyl-5,6,7,8- tetrahydroquinazolin-4-yl)- 2,3,4,5-tetrahydro-1,4- benzoxazepine |
| 225 | 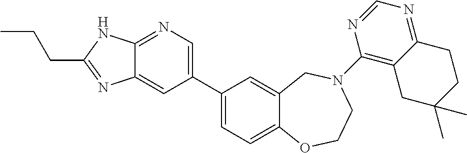 [SMILES: CCCc1nc2cc(-c3ccc4c(c3)CN(c3ncnc5c3CC(C)(C)CC5)CCO4)cnc2[nH]1] | 4-(6,6-dimethyl-5,6,7,8- tetrahydroquinazolin-4-yl)-7-(2- propyl-3H-imidazo[4,5- b]pyridin-6-yl)-2,3,4,5- tetrahydro-1,4-benzoxazepine |
| 226 | 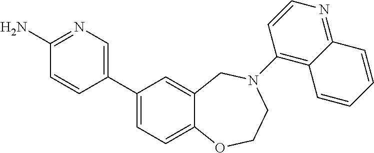 [SMILES: Nc1ccc(-c2ccc3c(c2)CN(c2ccnc4ccccc24)CCO3)cn1] | 5-(4-quinolin-4-yl-2,3,4,5- tetrahydro-1,4-benzoxazepin-7- yl)pyridin-2-amine |
| 227 | 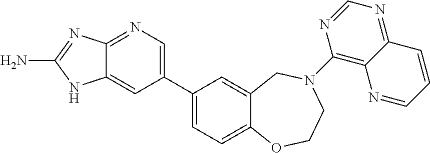 [SMILES: Nc1nc2ncc(-c3ccc4c(c3)CN(c3ncnc5cccnc35)CCO4)cc2[nH]1] | 6-(4-pyrido[3,2-d]pyrimidin-4- yl-2,3,4,5-tetrahydro-1,4- benzoxazepin-7-yl)-1H- imidazo[4,5-b]pyridin-2-amine |
| 228 | 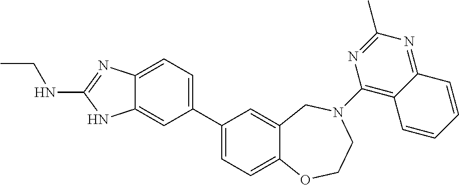 [SMILES: CCNc1nc2ccc(-c3ccc4c(c3)CN(c3nc(C)nc5ccccc35)CCO4)cc2[nH]1] | N-ethyl-6-[4-(2- methylquinazolin-4-yl)-2,3,4,5- tetrahydro-1,4-benzoxazepin-7- yl]-1H-benzimidazol-2-amine |
| 229 | 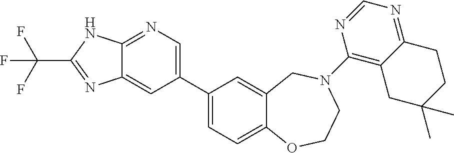 [SMILES: CC1(C)CCc2ncnc(N3CCOc4ccc(-c5cnc6[nH]c(C(F)(F)F)nc6c5)cc4C3)c2C1] | 7-[2-(difluoromethyl)-3H- imidazo[4,5-b]pyridin-6-yl]-4- (6,6-dimethyl-5,6,7,8- tetrahydroquinazolin-4-yl)- 2,3,4,5-tetrahydro-1,4- benzoxazepine |
| 230 | 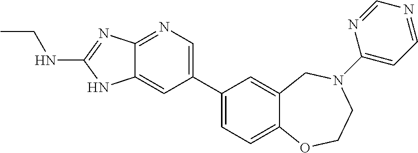 [SMILES: CCNc1nc2ncc(-c3ccc4c(c3)CN(c3ccncn3)CCO4)cc2[nH]1] | N-ethyl-6-(4-pyrimidin-4-yl- 2,3,4,5-tetrahydro-1,4- benzoxazepin-7-yl)-1H- imidazo[4,5-b]pyridin-2-amine |
| 231 | 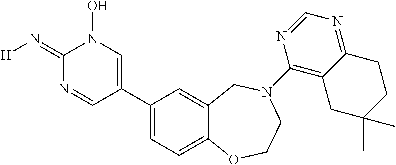 [SMILES: [H]/N=c1\ncc(-c2ccc3c(c2)CN(c2ncnc4c2CC(C)(C)CC4)CCO3)cn1O] | (2E)-5-[4-(6,6-dimethyl-5,6,7,8- tetrahydroquinazolin-4-yl)- 2,3,4,5-tetrahydro-1,4- benzoxazepin-7-yl]-2- iminopyrimidin-1(2H)-ol |
| 232 | 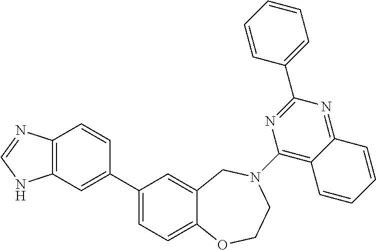 [SMILES: c1ccc(-c2nc(N3CCOc4ccc(-c5ccc6nc[nH]c6c5)cc4C3)c3ccccc3n2)cc1] | 7-(1H-benzimidazol-6-yl)-4-(2- phenylquinazolin-4-yl)-2,3,4,5- tetrahydro-1,4-benzoxazepine |
| 233 | 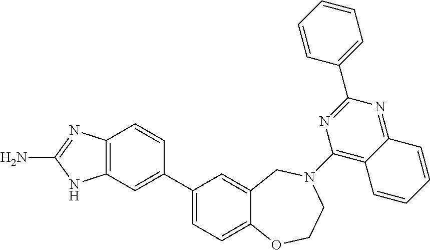 [SMILES: Nc1nc2ccc(-c3ccc4c(c3)CN(c3nc(-c5ccccc5)nc5ccccc35)CCO4)cc2[nH]1] | 6-[4-(2-phenylquinazolin-4-yl)- 2,3,4,5-tetrahydro-1,4- benzoxazepin-7-yl]-1H- benzimidazol-2-amine |
| 234 | 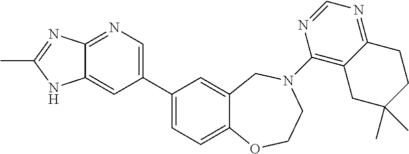 | 4-(6,6-dimethyl-5,6,7,8- tetrahydroquinazolin-4-yl)-7-(2- methyl-1H-imidazo[4,5- b]pyridin-6-yl)-2,3,4,5- tetrahydro-1,4-benzoxazepine |
| 235 | 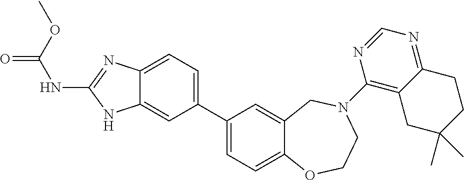 [SMILES: COC(=O)Nc1nc2ccc(-c3ccc4c(c3)CN(c3ncnc5c3CC(C)(C)CC5)CCO4)cc2[nH]1] | methyl {6-[4-(6,6-dimethyl- 5,6,7,8-tetrahydroquinazolin-4- yl)-2,3,4,5-tetrahydro-1,4- benzoxazepin-7-yl]-1H- benzimidazol-2-yl}carbamate |
| 236 | 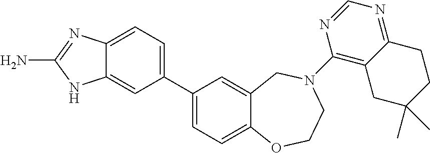 [SMILES: CC1(C)CCc2ncnc(N3CCOc4ccc(-c5ccc6nc(N)[nH]c6c5)cc4C3)c2C1] | 6-[4-(6,6-dimethyl-5,6,7,8- tetrahydroquinazolin-4-yl)- 2,3,4,5-tetrahydro-1,4- benzoxazepin-7-yl]-1H- benzimidazol-2-amine |
| 237 | 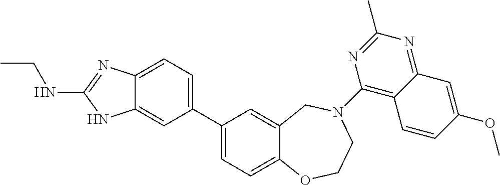 [SMILES: CCNc1nc2ccc(-c3ccc4c(c3)CN(c3nc(C)nc5cc(OC)ccc35)CCO4)cc2[nH]1] | N-ethyl-6-{4-[2-methyl-7- (methyloxy)quinazolin-4-yl]- 2,3,4,5-tetrahydro-1,4- benzoxazepin-7-yl}-1H- benzimidazol-2-amine |
| 238 | 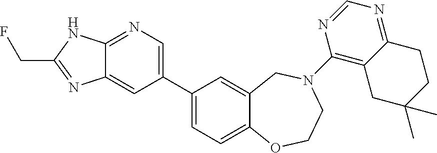 [SMILES: CC1(C)CCc2ncnc(N3CCOc4ccc(-c5cnc6[nH]c(CF)nc6c5)cc4C3)c2C1] | 4-(6,6-dimethyl-5,6,7,8- tetrahydroquinazolin-4-yl)-7-[2- (fluoromethyl)-3H-imidazo[4,5- b]pyridin-6-yl]-2,3,4,5- tetrahydro-1,4-benzoxazepine |
| 239 | 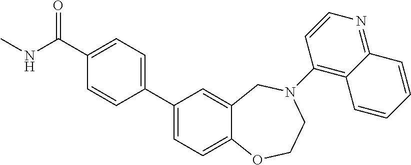 [SMILES: CNC(=O)c1ccc(-c2ccc3c(c2)CN(c2ccnc4ccccc24)CCO3)cc1] | N-methyl-4-(4-quinolin-4-yl- 2,3,4,5-tetrahydro-1,4- benzoxazepin-7-yl)benzamide |
| 240 | 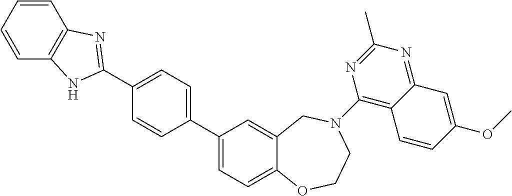 [SMILES: COc1ccc2c(N3CCOc4ccc(-c5ccc(-c6nc7ccccc7[nH]6)cc5)cc4C3)nc(C)nc2c1] | 7-[4-(1H-benzimidazol-2- yl)phenyl]-4-[2-methyl-7- (methyloxy)quinazolin-4-yl]- 2,3,4,5-tetrahydro-1,4- benzoxazepine |
| 241 | 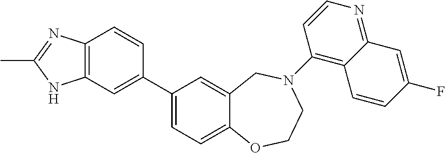 [SMILES: Cc1nc2ccc(-c3ccc4c(c3)CN(c3ccnc5cc(F)ccc35)CCO4)cc2[nH]1] | 4-(7-fluoroquinolin-4-yl)-7-(2- methyl-1H-benzimidazol-6-yl)- 2,3,4,5-tetrahydro-1,4- benzoxazepine |
| 242 | 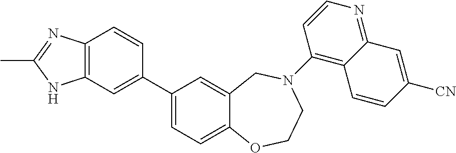 [SMILES: Cc1nc2ccc(-c3ccc4c(c3)CN(c3ccnc5cc(C#N)ccc35)CCO4)cc2[nH]1] | 4-[7-(2-methyl-1H- benzimidazol-6-yl)-2,3-dihydro- 1,4-benzoxazepin-4(5H)- yl]quinoline-7-carbonitrile |
| 243 | 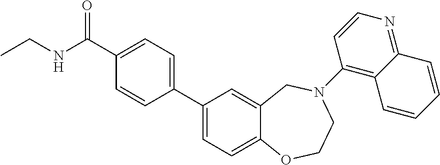 [SMILES: CCNC(=O)c1ccc(-c2ccc3c(c2)CN(c2ccnc4ccccc24)CCO3)cc1] | N-ethyl-4-(4-quinolin-4-yl- 2,3,4,5-tetrahydro-1,4- benzoxazepin-7-yl)benzamide |
| 244 | 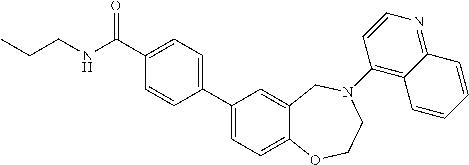 [SMILES: CCCNC(=O)c1ccc(-c2ccc3c(c2)CN(c2ccnc4ccccc24)CCO3)cc1] | N-propyl-4-(4-quinolin-4-yl- 2,3,4,5-tetrahydro-1,4- benzoxazepin-7-yl)benzamide |
| 245 | 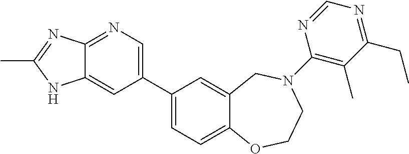 [SMILES: CCc1ncnc(N2CCOc3ccc(-c4cnc5nc(C)[nH]c5c4)cc3C2)c1C] | 4-(6-ethyl-5-methylpyrimidin-4- yl)-7-(2-methyl-1H-imidazo[4,5- b]pyridin-6-yl)-2,3,4,5- tetrahydro-1,4-benzoxazepine |
| 246 | 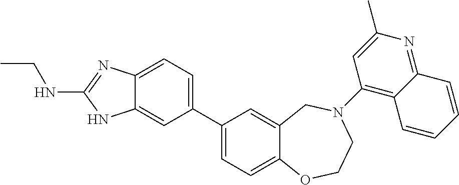 [SMILES: CCNc1nc2ccc(-c3ccc4c(c3)CN(c3cc(C)nc5ccccc35)CCO4)cc2[nH]1] | N-ethyl-6-[4-(2-methylquinolin- 4-yl)-2,3,4,5-tetrahydro-1,4- benzoxazepin-7-yl]-1H- benzimidazol-2-amine |
| 247 | 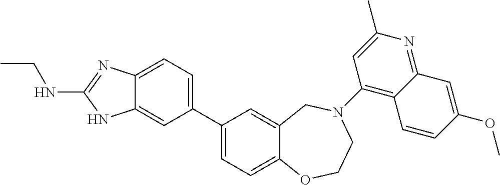 [SMILES: CCNc1nc2ccc(-c3ccc4c(c3)CN(c3cc(C)nc5cc(OC)ccc35)CCO4)cc2[nH]1] | N-ethyl-6-{4-[2-methyl-7- (methyloxy)quinolin-4-yl]- 2,3,4,5-tetrahydro-1,4- benzoxazepin-7-yl}-1H- benzimidazol-2-amine |
| 248 | 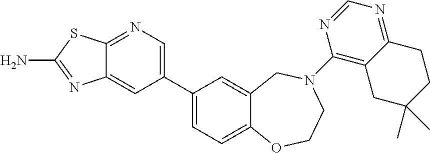 [SMILES: CC1(C)CCc2ncnc(N3CCOc4ccc(-c5cnc6sc(N)nc6c5)cc4C3)c2C1] | 6-[4-(6,6-dimethyl-5,6,7,8- tetrahydroquinazolin-4-yl)- 2,3,4,5-tetrahydro-1,4- benzoxazepin-7- yl][1,3]thiazolo[5,4-b]pyridin-2- amine |
| 249 | 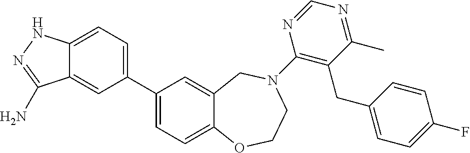 [SMILES: Cc1ncnc(N2CCOc3ccc(-c4ccc5[nH]nc(N)c5c4)cc3C2)c1Cc1ccc(F)cc1] | 5-(4-{5-[(4- fluorophenyl)methyl]-6- methylpyrimidin-4-yl}-2,3,4,5- tetrahydro-1,4-benzoxazepin-7- yl)-1H-indazol-3-amine |
| 250 | 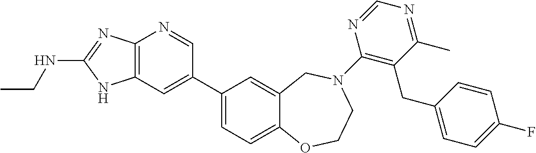 [SMILES: CCNc1nc2ncc(-c3ccc4c(c3)CN(c3ncnc(C)c3Cc3ccc(F)cc3)CCO4)cc2[nH]1] | N-ethyl-6-(4-{5-[(4- fluorophenyl)methyl]-6- methylpyrimidin-4-yl}-2,3,4,5- tetrahydro-1,4-benzoxazepin-7- yl)-1H-imidazo[4,5-b]pyridine-2- amine |
| 251 | 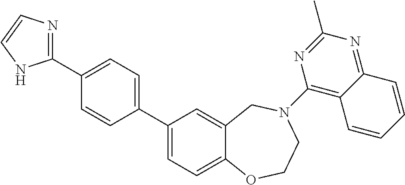 [SMILES: Cc1nc(N2CCOc3ccc(-c4ccc(-c5ncc[nH]5)cc4)cc3C2)c2ccccc2n1] | 7-[4-(1H-imidazol-2-yl)phenyl]- 4-(2-methylquinazolin-4-yl)- 2,3,4,5-tetrahydro-1,4- benzoxazepine |
| 252 | 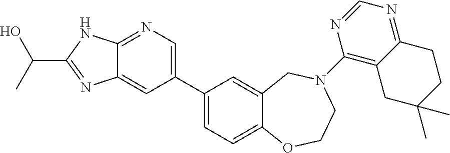 [SMILES: CC(O)c1nc2cc(-c3ccc4c(c3)CN(c3ncnc5c3CC(C)(C)CC5)CCO4)cnc2[nH]1] | 1-{6-[4-(6,6-dimethyl-5,6,7,8- tetrahydroquinazolin-4-yl)- 2,3,4,5-tetrahydro-1,4- benzoxazepin-7-yl]-3H- imidazo[4,5-b]pyridin-2- yl}ethanol |
| 253 | 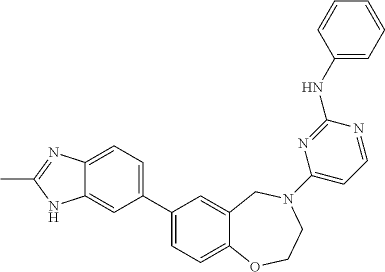 [SMILES: Cc1nc2ccc(-c3ccc4c(c3)CN(c3ccnc(Nc5ccccc5)n3)CCO4)cc2[nH]1] | 4-[7-(2-methyl-1H- benzimidazol-6-yl)-2,3-dihydro- 1,4-benzoxazepin-4(5H)-yl]-N- phenylpyrimidin-2-amine |
| 254 | 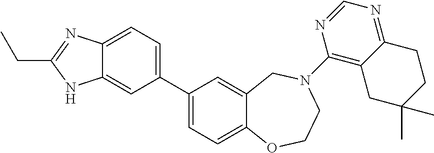 [SMILES: CCc1nc2ccc(-c3ccc4c(c3)CN(c3ncnc5c3CC(C)(C)CC5)CCO4)cc2[nH]1] | 4-(6,6-dimethyl-5,6,7,8- tetrahydroquinazolin-4-yl)-7-(2- ethyl-1H-benzimidazol-6-yl)- 2,3,4,5-tetrahydro-1,4- benzoxazepine |
| 255 | 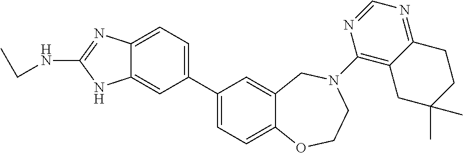 [SMILES: CCNc1nc2ccc(-c3ccc4c(c3)CN(c3ncnc5c3CC(C)(C)CC5)CCO4)cc2[nH]1] | 6-[4-(6,6-dimethyl-5,6,7,8- tetrahydroquinazolin-4-yl)- 2,3,4,5-tetrahydro-1,4- benzoxazepin-7-yl]-N-ethyl-1H- benzimidazol-2-amine |
| 256 | 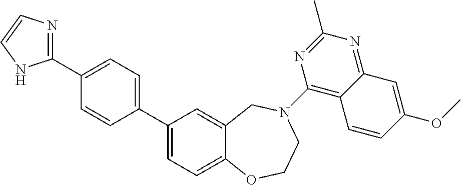 [SMILES: COc1ccc2c(N3CCOc4ccc(-c5ccc(-c6ncc[nH]6)cc5)cc4C3)nc(C)nc2c1] | 7-[4-(1H-imidazol-2-yl)phenyl]- 4-[2-methyl-7- (methyloxy)quinazolin-4-yl]- 2,3,4,5-tetrahydro-1,4- benzoxazepine |
| 257 | 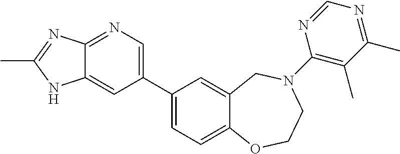 | 4-(5,6-dimethylpyrimidin-4-yl)- 7-(2-methyl-1H-imidazo[4,5- b]pyridin-6-yl)-2,3,4,5- tetrahydro-1,4-benzoxazepine |
| 258 | 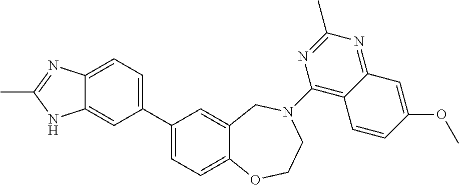 | 7-(2-methyl-1H-benzimidazol-6- yl)-4-[2-methyl-7- (methyloxy)quinazolin-4-yl]- 2,3,4,5-tetrahydro-1,4- benzoxazepine |
| 259 | 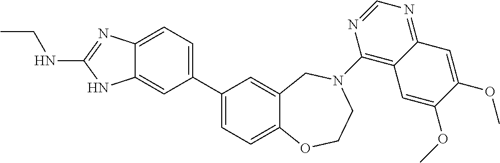 [SMILES: CCNc1nc2ccc(-c3ccc4c(c3)CN(c3ncnc5cc(OC)c(OC)cc35)CCO4)cc2[nH]1] | 6-{4-[6,7- bis(methyloxy)quinazolin-4-yl]- 2,3,4,5-tetrahydro-1,4- benzoxazepin-7-yl}-N-ethyl-1H- benzimidazol-2-amine |
| 260 | 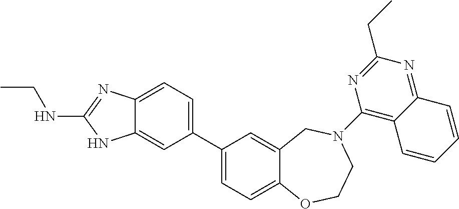 [SMILES: CCNc1nc2ccc(-c3ccc4c(c3)CN(c3nc(CC)nc5ccccc35)CCO4)cc2[nH]1] | N-ethyl-6-[4-(2-ethylquinazolin- 4-yl)-2,3,4,5-tetrahydro-1,4- benzoxazepin-7-yl]-1H- benzimidazol-2-amine |
| 261 | 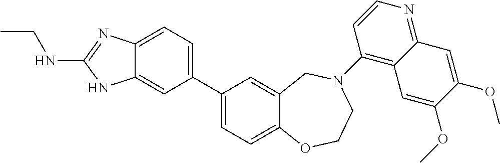 [SMILES: CCNc1nc2ccc(-c3ccc4c(c3)CN(c3ccnc5cc(OC)c(OC)cc35)CCO4)cc2[nH]1] | 6-{4-[6,7- bis(methyloxy)quinazolin-4-yl]- 2,3,4,5-tetrahydro-1,4- benzoxazepin-7-yl}-N-ethyl-1H- benzimidazol-2-amine |
| 262 | 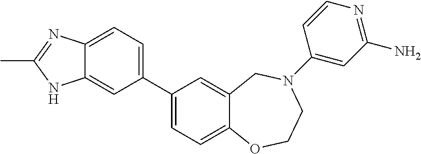 [SMILES: Cc1nc2ccc(-c3ccc4c(c3)CN(c3ccnc(N)c3)CCO4)cc2[nH]1] | 4-[7-(2-methyl-1H- benzimidazol-6-yl)-2,3-dihydro- 1,4-benzoxazepin-4(5H)- yl]pyridin-2-amine |
| 263 | 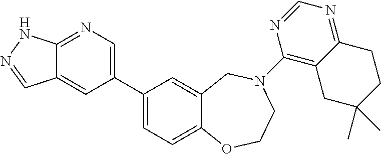 [SMILES: CC1(C)CCc2ncnc(N3CCOc4ccc(-c5cnc6[nH]ncc6c5)cc4C3)c2C1] | 4-(6,6-dimethyl-5,6,7,8- tetrahydroquinazolin-4-yl)-7- (1H-pyrazolo[3,4-b]pyridin-5- yl)-2,3,4,5-tetrahydro-1,4- benzoxazepine |
| 264 | 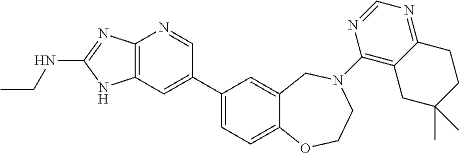 [SMILES: CCNc1nc2ncc(-c3ccc4c(c3)CN(c3ncnc5c3CC(C)(C)CC5)CCO4)cc2[nH]1] | 6-[4-(6,6-dimethyl-5,6,7,8- tetrahydroquinazolin-4-yl)- 2,3,4,5-tetrahydro-1,4- benzoxazepin-7-yl]-N-ethyl-1H- imidazo[4,5-b]pyridin-2-amine |
| 265 | 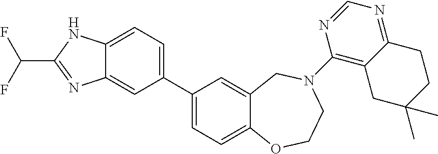 [SMILES: CC1(C)CCc2ncnc(N3CCOc4ccc(-c5ccc6[nH]c(C(F)F)nc6c5)cc4C3)c2C1] | 7-[2-(difluoromethyl)-1H- benzimidazol-5-yl]-4-(6,6- dimethyl-5,6,7,8- tetrahydroquinazolin-4-yl)- 2,3,4,5-tetrahydro-1,4- benzoxazepine |
| 266 | 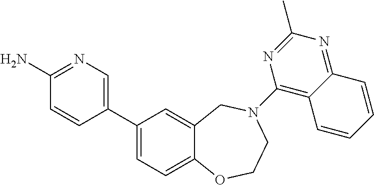 [SMILES: Cc1nc(N2CCOc3ccc(-c4ccc(N)nc4)cc3C2)c2ccccc2n1] | 5-[4-(2-methylquinazolin-4-yl)- 2,3,4,5-tetrahydro-1,4- benzoxazepin-7-yl]pyridin-2- amine |
| 267 | 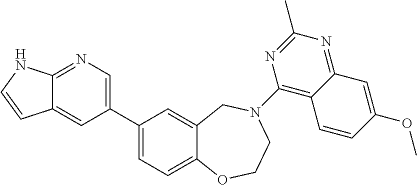 [SMILES: COc1ccc2c(N3CCOc4ccc(-c5cnc6[nH]ccc6c5)cc4C3)nc(C)nc2c1] | 4-[2-methyl-7- (methyloxy)quinazolin-4-yl]-7- (1H-pyrrolo[2,3-b]pyridin-5-yl)- 2,3,4,5-tetrahydro-1,4- benzoxazepine |
| 268 | 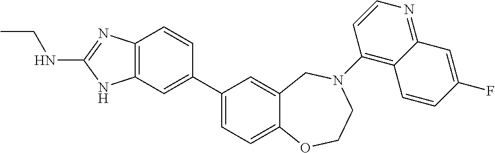 [SMILES: CCNc1nc2ccc(-c3ccc4c(c3)CN(c3ccnc5cc(F)ccc35)CCO4)cc2[nH]1] | N-ethyl-6-[4-(7-fluoroquinolin- 4-yl)-2,3,4,5-tetrahydro-1,4- benzoxazepin-7-yl]-1H- benzimidazol-2-amine |
| 269 | 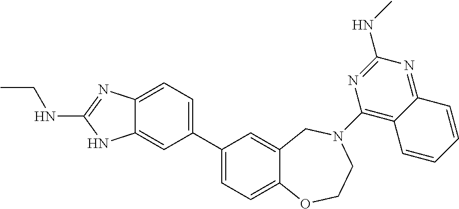 [SMILES: CCNc1nc2ccc(-c3ccc4c(c3)CN(c3nc(NC)nc5ccccc35)CCO4)cc2[nH]1] | 4-{7-[2-(ethylamino)-1H- benzimidazol-6-yl]-2,3-dihydro- 1,4-benzoxazepin-4(5H)-yl}-N- methylquinazolin-2-amine |
| 270 | 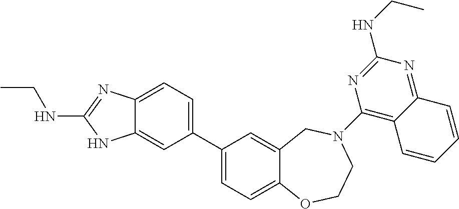 [SMILES: CCNc1nc(N2CCOc3ccc(-c4ccc5nc(NCC)[nH]c5c4)cc3C2)c2ccccc2n1] | N-ethyl-4-{7-[2-(ethylamino)- 1H-benzimidazol-6-yl]-2,3- dihydro-1,4-benzoxazepin- 4(5H)-yl}quinazolin-2-amine |
| 271 | 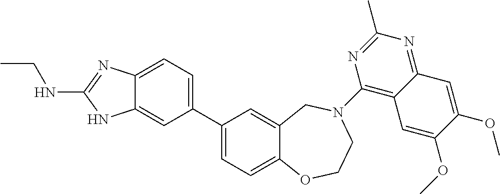 [SMILES: CCNc1nc2ccc(-c3ccc4c(c3)CN(c3nc(C)nc5cc(OC)c(OC)cc35)CCO4)cc2[nH]1] | N-ethyl-6-{4-[2-methyl-6,7- bis(methyloxy)quinazolin-4-yl]- 2,3,4,5-tetrahydro-1,4- benzoxazepin-7-yl}-1H- benzimidazol-2-amine |
| 272 | 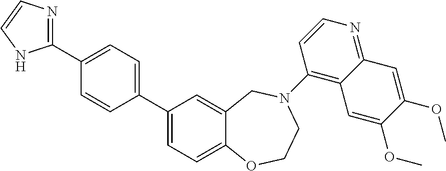 [SMILES: COc1cc2nccc(N3CCOc4ccc(-c5ccc(-c6ncc[nH]6)cc5)cc4C3)c2cc1OC] | 4-[6,7-bis(methyloxy)quinolin- 4-yl]-7-[4-(1H-imidazol-2- yl)phenyl]-2,3,4,5-tetrahydro- 1,4-benzoxazepine |
| 273 | 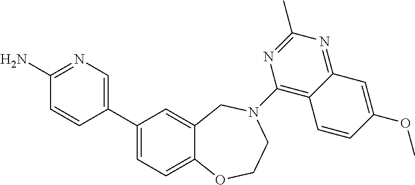 [SMILES: COc1ccc2c(N3CCOc4ccc(-c5ccc(N)nc5)cc4C3)nc(C)nc2c1] | 5-{4-[2-methyl-7- (methyloxy)quinazolin-4-yl]- 2,3,4,5-tetrahydro-1,4- benzoxazepin-7-yl}pyridin-2- amine |
| 274 | 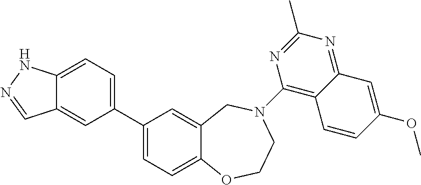 [SMILES: COc1ccc2c(N3CCOc4ccc(-c5ccc6[nH]ncc6c5)cc4C3)nc(C)nc2c1] | 7-(1H-indazol-5-yl)-4-[2- methyl-7- (methyloxy)quinazolin-4-yl]- 2,3,4,5-tetrahydro-1,4- benzoxazepine |
| 275 | 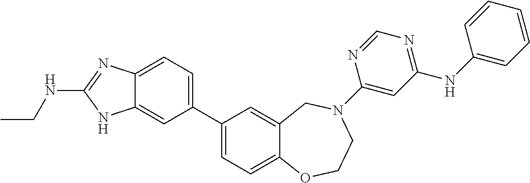 [SMILES: CCNc1nc2ccc(-c3ccc4c(c3)CN(c3cc(Nc5ccccc5)ncn3)CCO4)cc2[nH]1] | N-ethyl-6-{4-[6- (phenylamino)pyrimidin-4-yl]- 2,3,4,5-tetrahydro-1,4- benzoxazepin-7-yl}-1H- benzimidazol-2-amine |
| 276 | 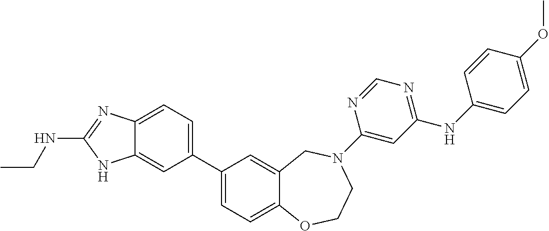 [SMILES: CCNc1nc2ccc(-c3ccc4c(c3)CN(c3cc(Nc5ccc(OC)cc5)ncn3)CCO4)cc2[nH]1] | N-ethyl-6-[4-(6-{[4- (methyloxy)phenyl]amino} pyrimidin-4-yl)-2,3,4,5- tetrahydro-1,4- benzoxazepin-7-yl]-1H- benzimidazol-2-amine |
| 277 | 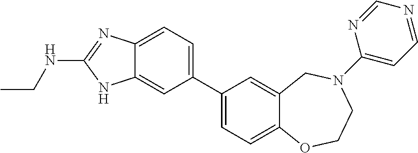 [SMILES: CCNc1nc2ccc(-c3ccc4c(c3)CN(c3ccncn3)CCO4)cc2[nH]1] | N-ethyl-6-(4-pyrimidin-4-yl- 2,3,4,5-tetrahydro-1,4- benzoxazepin-7-yl)-1H- benzimidazol-2-amine |
| 278 | 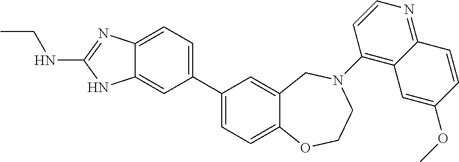 [SMILES: CCNc1nc2ccc(-c3ccc4c(c3)CN(c3ccnc5ccc(OC)cc35)CCO4)cc2[nH]1] | N-ethyl-6-{4-[6- (methyloxy)quinolin-4-yl]- 2,3,4,5-tetrahydro-1,4- benzoxazepin-7-yl)-1H- benzimidazol-2-amine |
| 279 | 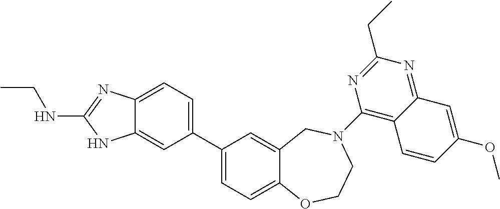 [SMILES: CCNc1nc2ccc(-c3ccc4c(c3)CN(c3nc(CC)nc5cc(OC)ccc35)CCO4)cc2[nH]1] | N-ethyl-6-{4-[2-ethyl-7- (methyloxy)quinazolin-4-yl]- 2,3,4,5-tetrahydro-1,4- benzoxazepin-7-yl}-1H- benzimidazol-2-amine |
| 280 | 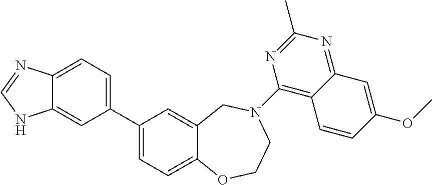 [SMILES: COc1ccc2c(N3CCOc4ccc(-c5ccc6nc[nH]c6c5)cc4C3)nc(C)nc2c1] | 7-(1H-benzimidazol-6-yl)-4-[2- methyl-7- (methyloxy)quinazolin-4-yl]- 2,3,4,5-tetrahydro-1,4- benzoxazepine |
| 281 | 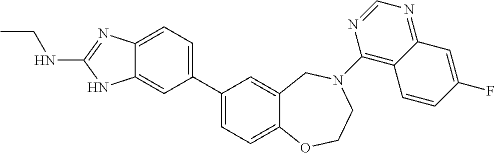 [SMILES: CCNc1nc2ccc(-c3ccc4c(c3)CN(c3ncnc5cc(F)ccc35)CCO4)cc2[nH]1] | N-ethyl-6-[4-(7- fluoroquinazolin-4-yl)-2,3,4,5- tetrahydro-1,4-benzoxazepin-7- yl]-1H-benzimidazol-2-amine |
| 282 | 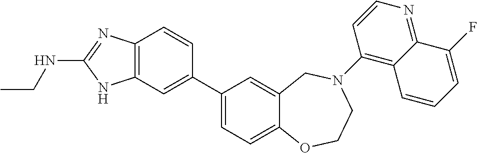 [SMILES: CCNc1nc2ccc(-c3ccc4c(c3)CN(c3ccnc5c(F)cccc35)CCO4)cc2[nH]1] | N-ethyl-6-[4-(8-fluoroquinolin- 4-yl)-2,3,4,5-tetrahydro-1,4- benzoxazepin-7-yl]-1H- benzimidazol-2-amine |
| 283 | 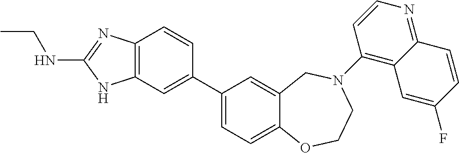 [SMILES: CCNc1nc2ccc(-c3ccc4c(c3)CN(c3ccnc5ccc(F)cc35)CCO4)cc2[nH]1] | N-ethyl-6-[4-(6-fluoroquinolin- 4-yl)-2,3,4,5-tetrahydro-1,4- benzoxazepin-7-yl]-1H- benzimidazol-2-amine |
| 284 | 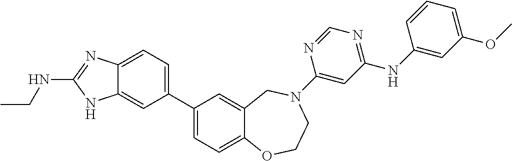 [SMILES: CCNc1nc2ccc(-c3ccc4c(c3)CN(c3cc(Nc5cccc(OC)c5)ncn3)CCO4)cc2[nH]1] | N-ethyl-6-[4-(6-{[3- (methyloxy)phenyl]amino}pyri- midin-4-yl)-2,3,4,5-tetrahydro- 1,4-benzoxazepin-7-yl]-1H- benzimidazol-2-amine |
| 285 | 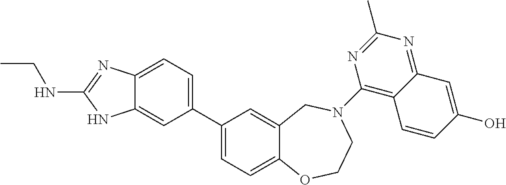 [SMILES: CCNc1nc2ccc(-c3ccc4c(c3)CN(c3nc(C)nc5cc(O)ccc35)CCO4)cc2[nH]1] | 4-{7-[2-(ethylamino)-1H- benzimidazol-6-yl]-2,3-dihydro- 1,4-benzoxazepin-4(5H)-yl}-2- methylquinazolin-7-ol |
| 286 | 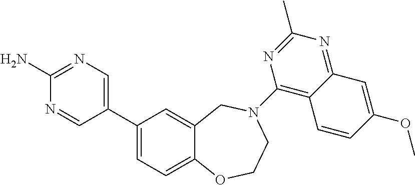 [SMILES: COc1ccc2c(N3CCOc4ccc(-c5cnc(N)nc5)cc4C3)nc(C)nc2c1] | 5-{4-[2-methyl-7- (methyloxy)quinazolin-4-yl]- 2,3,4,5-tetrahydro-1,4- benzoxazepin-7-yl}pyrimidin-2- amine |
| 287 | 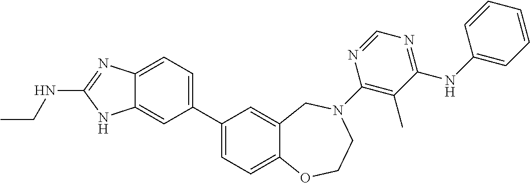 [SMILES: CCNc1nc2ccc(-c3ccc4c(c3)CN(c3ncnc(Nc5ccccc5)c3C)CCO4)cc2[nH]1] | N-ethyl-6-{4-[5-methyl-6- (phenylamino)pyrimidin-4-yl]- 2,3,4,5-tetrahydro-1,4- benzoxazepin-7-yl}-1H- benzimidazol-2-amine |
| 288 | 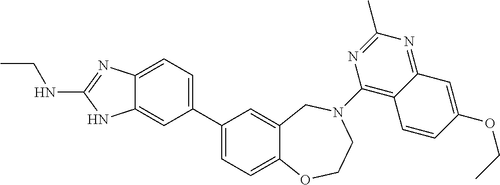 [SMILES: CCNc1nc2ccc(-c3ccc4c(c3)CN(c3nc(C)nc5cc(OCC)ccc35)CCO4)cc2[nH]1] | N-ethyl-6-{4-[7-(ethyloxy)-2- methylquinazolin-4-yl]-2,3,4,5- tetrahydro-1,4-benzoxazepin-7- yl}-1H-benzimidazol-2-amine |
| 289 | 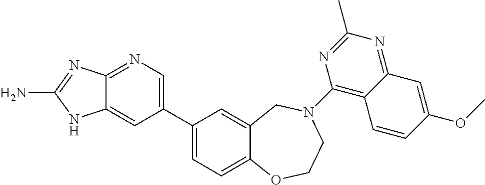 [SMILES: COc1ccc2c(N3CCOc4ccc(-c5cnc6nc(N)[nH]c6c5)cc4C3)nc(C)nc2c1] | 6-{4-[2-methyl-7- (methyloxy)quinazolin-4-yl]- 2,3,4,5-tetrahydro-1,4- benzoxazepin-7-yl}-1H- imidazo[4,5-b]pyridin-2-amine |
| 290 | 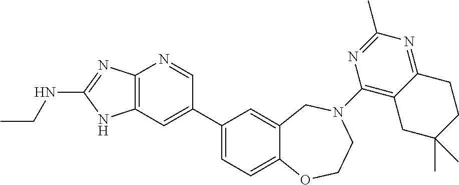 [SMILES: CCNc1nc2ncc(-c3ccc4c(c3)CN(c3nc(C)nc5c3CC(C)(C)CC5)CCO4)cc2[nH]1] | N-ethyl-6-[4-(2,6,6-trimethyl- 5,6,7,8-tetrahydroquinazolin-4- yl)-2,3,4,5-tetrahydro-1,4- benzoxazepin-7-yl]- 1H- imidazo[4,5-b]pyridin-2-amine |
| 291 | 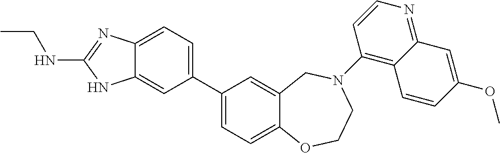 [SMILES: CCNc1nc2ccc(-c3ccc4c(c3)CN(c3ccnc5cc(OC)ccc35)CCO4)cc2[nH]1] | N-ethyl-6-{4-[7- (methyloxy)quinolin-4-yl]- 2,3,4,5-tetrahydro-1,4- benzoxazepin-7-yl}-1H- benzimidazol-2-amine |
| 292 | 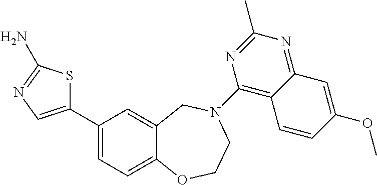 [SMILES: COc1ccc2c(N3CCOc4ccc(-c5cnc(N)s5)cc4C3)nc(C)nc2c1] | 5-{4-[2-methyl-7- (methyloxy)quinazolin-4-yl]- 2,3,4,5-tetrahydro-1,4- benzoxazepin-7-yl}-1,3-thiazol- 2-amine |
| 293 | 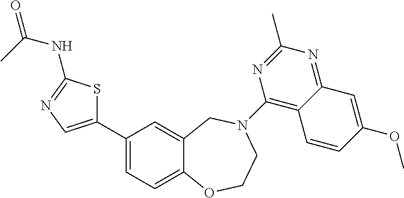 [SMILES: COc1ccc2c(N3CCOc4ccc(-c5cnc(NC(C)=O)s5)cc4C3)nc(C)nc2c1] | N-5-{4-[2-methyl-7- (methyloxy)quinazolin-4-yl]- 2,3,4,5-tetrahydro-1,4- benzoxazepin-7-yl}-1,3-thiazol- 2-yl)acetamide |
| 294 | 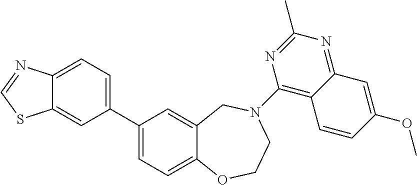 [SMILES: COc1ccc2c(N3CCOc4ccc(-c5ccc6ncsc6c5)cc4C3)nc(C)nc2c1] | 7-(1,3-benzothiazol-6-yl)-4-[2- methyl-7- (methyloxy)quinazolin-4-yl]- 2,3,4,5-tetrahydro-1,4- benzoxazepine |
| 295 | 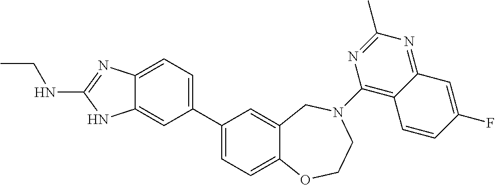 [SMILES: CCNc1nc2ccc(-c3ccc4c(c3)CN(c3nc(C)nc5cc(F)ccc35)CCO4)cc2[nH]1] | N-ethyl-6-[4-(7-fluoro-2- methylquinazolin-4-yl)-2,3,4,5- tetrahydro-1,4-benzoxazepin-7- yl]-1H-benzimidazol-2-amine |
| 296 | 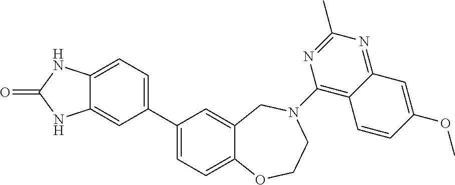 [SMILES: COc1ccc2c(N3CCOc4ccc(-c5ccc6[nH]c(=O)[nH]c6c5)cc4C3)nc(C)nc2c1] | 5-{4-[2-methyl-7- (methyloxy)quinazolin-4-yl]- 2,3,4,5-tetrahydro-1,4- benzoxazepin-7-yl}-1,3-dihydro- 2H-benzimidazol-2-one |
| 297 | 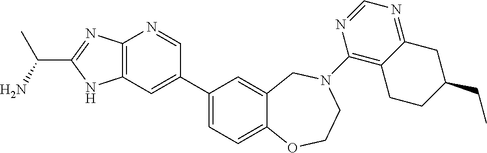 [SMILES: CC[C@H]1CCc2c(ncnc2N2CCOc3ccc(-c4cnc5nc([C@@H](C)N)[nH]c5c4)cc3C2)C1] | (1R)-1-(6-{4-[(7S)-7-ethyl- 5,6,7,8-tetrahydroquinazolin-4- yl]-2,3,4,5-tetrahydro-1,4- benzoxazepin-7-yl}-1H- imidazo[4,5-b]pyridin-2- yl)ethanamine |
| 298 | 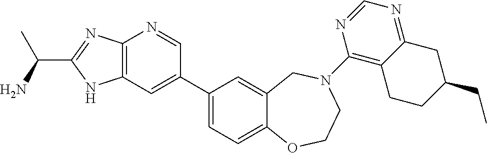 [SMILES: CC[C@H]1CCc2c(ncnc2N2CCOc3ccc(-c4cnc5nc([C@H](C)N)[nH]c5c4)cc3C2)C1] | (1S)-1-(6-{4-[(7S)-7-ethyl- 5,6,7,8-tetrahydroquinazolin-4- yl]-2,3,4,5-tetrahydro-1,4- benzoxazepin-7-yl}-1H- imidazo[4,5-b]pyridin-2- yl)ethanamine |
| 299 | 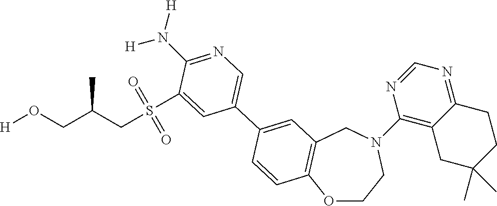 [SMILES: C[C@H](CO)CS(=O)(=O)c1cc(-c2ccc3c(c2)CN(c2ncnc4c2CC(C)(C)CC4)CCO3)cnc1N] | (2R)-3-({2-amino-5-[4-(6,6- dimethyl-5,6,7,8- tetrahydroquinazolin-4-yl)- 2,3,4,5-tetrahydro-1,4- benzoxazepin-7-yl]pyridin-3- yl}sulfonyl)-2-methylpropan-1- ol |
| 300 | 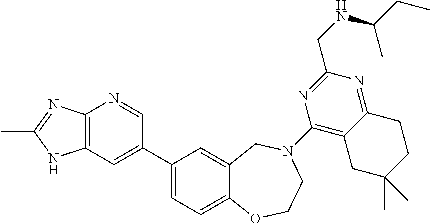 [SMILES: CC[C@@H](C)NCc1nc2c(c(N3CCOc4ccc(-c5cnc6nc(C)[nH]c6c5)cc4C3)n1)CC(C)(C)CC2] | (2R)-N-({6,6-dimethyl-4-[7-(2- methyl-1H-imidazo[4,5- b]pyridin-6-yl)-2,3-dihydro-1,4- benzoxazepin-4(5H)-yl]-5,6,7,8- tetrahydroquinazolin-2- yl}methyl)butan-2-amine |
| 301 | 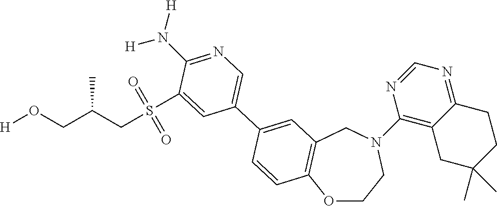 [SMILES: C[C@@H](CO)CS(=O)(=O)c1cc(-c2ccc3c(c2)CN(c2ncnc4c2CC(C)(C)CC4)CCO3)cnc1N] | (2S)-3-({2-amino-5-[4-(6,6- dimethyl-5,6,7,8- tetrahydroquinazolin-4-yl)- 2,3,4,5-tetrahydro-1,4- benzoxazepin-7-yl] pyridin-3- yl}sulfonyl)-2-methylpropan-1- ol |
| 302 | 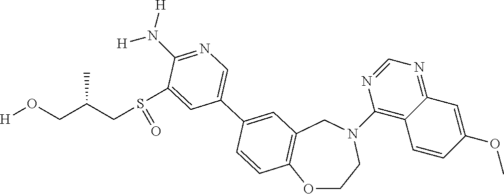 [SMILES: COc1ccc2c(N3CCOc4ccc(-c5cnc(N)c(S(=O)C[C@@H](C)CO)c5)cc4C3)ncnc2c1] | (2S)-3-[(2-amino-5-{4-[7- (methyloxy)quinazolin-4-yl]- 2,3,4,5-tetrahydro-1,4- benzoxazepin-7-yl} pyridin-3- yl)sulfinyl]-2-methylpropan-1-ol |
| 303 | 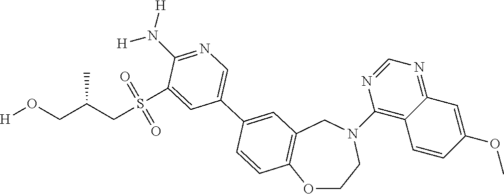 [SMILES: COc1ccc2c(N3CCOc4ccc(-c5cnc(N)c(S(=O)(=O)C[C@@H](C)CO)c5)cc4C3)ncnc2c1] | (2S)-3-[(2-amino-5-{4-[7- (methyloxy)quinazolin-4-yl]- 2,3,4,5-tetrahydro-1,4- benzoxazepin-7-yl} pyridin-3- yl)sulfonyl]-2-methylpropan-1- ol |
| 304 | 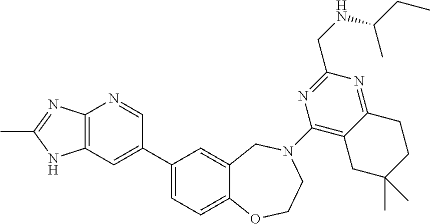 [SMILES: CC[C@H](C)NCc1nc2c(c(N3CCOc4ccc(-c5cnc6nc(C)[nH]c6c5)cc4C3)n1)CC(C)(C)CC2] | (2S)-N-({6,6-dimethyl-4-[7-(2- methyl-1H-imidazo[4,5- b]pyridin-6-yl)-2,3-dihydro-1,4- benzoxazepin-4(5H)-yl]-5,6,7,8- tetrahydroquinazolin-2- yl}methyl)butan-2-amine |
| 305 | 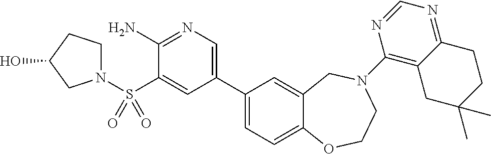 [SMILES: CC1(C)CCc2ncnc(N3CCOc4ccc(-c5cnc(N)c(S(=O)(=O)N6CC[C@@H](O)C6)c5)cc4C3)c2C1] | (3R)-1-({2-amino-5-[4-(6,6- dimethyl-5,6,7,8- tetrahydroquinazolin-4-yl)- 2,3,4,5-tetrahydro-1,4- benzoxazepin-7-yl]pyridin-3- yl}sulfonyl)pyrrolidin-3-ol |
| 306 | 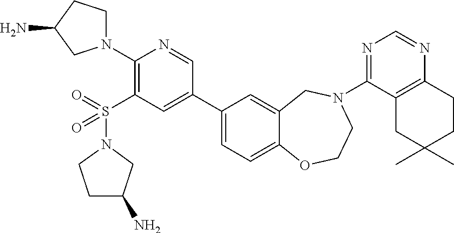 [SMILES: CC1(C)CCc2ncnc(N3CCOc4ccc(-c5cnc(N6CC[C@H](N)C6)c(S(=O)(=O)N6CC[C@H](N)C6)c5)cc4C3)c2C1] | (3S)-1-({2-[(3S)-3- aminopyrrolidin-1-yl)-5-[4-(6,6- dimethyl-5,6,7,8- tetrahydroquinazolin-4-yl)- 2,3,4,5-tetrahydro-1,4- benzoxazepin-7-yl]pyridin-3- yl}sulfonyl)pyrrolidin-3-amine |
| 307 | 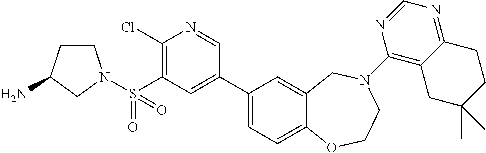 [SMILES: CC1(C)CCc2ncnc(N3CCOc4ccc(-c5cnc(Cl)c(S(=O)(=O)N6CC[C@H](N)C6)c5)cc4C3)c2C1] | (3S)-1-({2-chloro-5-[4-(6,6- dimethyl-5,6,7,8- tetrahydroquinazolin-4-yl)- 2,3,4,5-tetrahydro-1,4- benzoxazepin-7-yl]pyridin-3- yl}sulfonyl)pyrrolidin-3-amine |
| 308 | 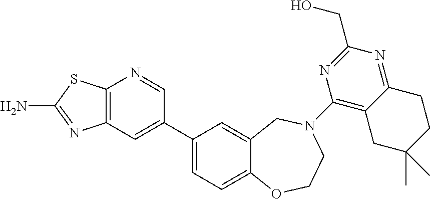 [SMILES: CC1(C)CCc2nc(CO)nc(N3CCOc4ccc(-c5cnc6sc(N)nc6c5)cc4C3)c2C1] | {4-[7-(2-amino[1,3]thiazolo[5,4- b]pyridin-6-yl)-2,3-dihydro-1,4- benzoxazepin-4(5H)-yl]-6,6- dimethyl-5,6,7,8- tetrahydroquinazolin-2- yl}methanol |
| 309 | 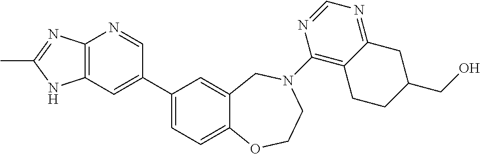 [SMILES: Cc1nc2ncc(-c3ccc4c(c3)CN(c3ncnc5c3CCC(CO)C5)CCO4)cc2[nH]1] | {4-[7-(2-methyl-1H- imidazo[4,5-b]pyridin-6-yl)-2,3- dihydro-1,4-benzoxazepin- 4(5H)-yl]-5,6,7,8- tetrahydroquinazolin-7- yl}methanol |
| 310 | 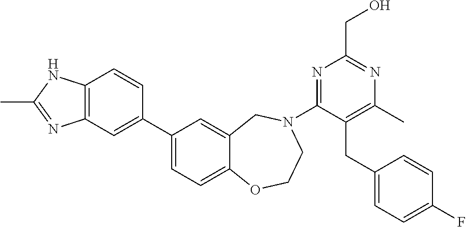 [SMILES: Cc1nc2cc(-c3ccc4c(c3)CN(c3nc(CO)nc(C)c3Cc3ccc(F)cc3)CCO4)ccc2[nH]1] | {5-[(4-fluorophenyl)methyl]-4- methyl-6-[7-(2-methyl-1H- benzimidazol-5-yl)-2,3-dihydro- 1,4-benzoxazepin-4(5H)- yl]pyrimidin-2-yl}methanol |
| 311 | 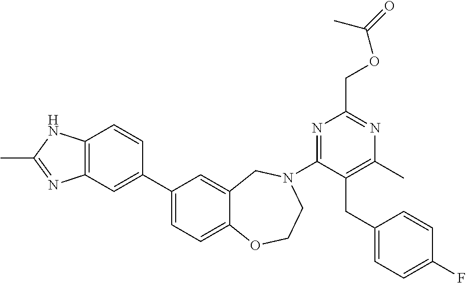 [SMILES: CC(=O)OCc1nc(C)c(Cc2ccc(F)cc2)c(N2CCOc3ccc(-c4ccc5[nH]c(C)nc5c4)cc3C2)n1] | {5-[(4-fluorophenyl)methyl]-4- methyl-6-[7-(2-methyl-1H- benzimidazol-5-yl)-2,3-dihydro- 1,4-benzoxazepin-4(5H)- yl]pyrimidin-2-yl}methyl acetate |
| 312 | 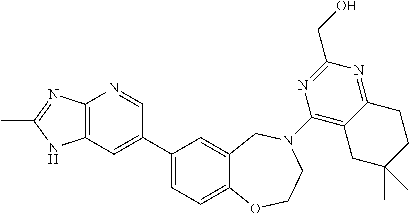 | {6,6-dimethyl-4-[7-(2-methyl- 1H-imidazo[4,5-b]pyridin-6-yl)- 2,3-dihydro-1,4-benzoxazepin- 4(5H)-yl]-5,6,7,8- tetrahydroquinazolin-2- yl}methanol |
| 313 | 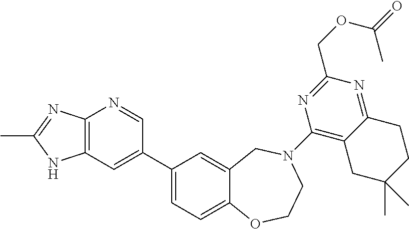 | {6,6-dimethyl-4-[7-(2-methyl- 1H-imidazo[4,5-b]pyridin-6-yl)- 2,3-dihydro-1,4-benzoxazepin- 4(5H)-yl]-5,6,7,8- tetrahydroquinazolin-2- yl}methyl acetate |
| 314 | 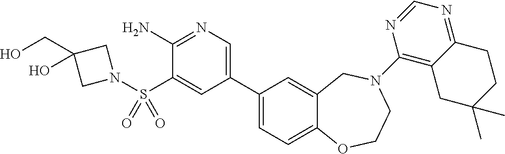 [SMILES: CC1(C)CCc2ncnc(N3CCOc4ccc(-c5cnc(N)c(S(=O)(=O)N6CC(O)(CO)C6)c5)cc4C3)c2C1] | 1-({2-amino-5-[4-(6,6-dimethyl- 5,6,7,8-tetrahydroquinazolin-4- yl)-2,3,4,5-tetrahydro-1,4- benzoxazepin-7-yl]pyridin-3- yl}sulfonyl)-3- (hydroxymethyl)azetidin-3-ol |
| 315 | 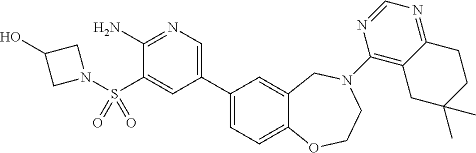 [SMILES: CC1(C)CCc2ncnc(N3CCOc4ccc(-c5cnc(N)c(S(=O)(=O)N6CC(O)C6)c5)cc4C3)c2C1] | 1-({2-amino-5-[4-(6,6-dimethyl- 5,6,7,8-tetrahydroquinazolin-4- yl)-2,3,4,5-tetrahydro-1,4- benzoxazepin-7-yl]pyridin-3- yl}sulfonyl)azetidin-3-ol |
| 316 | 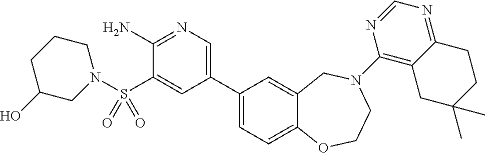 [SMILES: CC1(C)CCc2ncnc(N3CCOc4ccc(-c5cnc(N)c(S(=O)(=O)N6CCCC(O)C6)c5)cc4C3)c2C1] | 1-({2-amino-5-[4-(6,6-dimethyl- 5,6,7,8-tetrahydroquinazolin-4- yl)-2,3,4,5-tetrahydro-1,4- benzoxazepin-7-yl]pyridin-3- yl}sulfonyl)piperidin-3-ol |
| 317 | 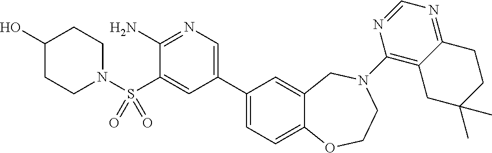 [SMILES: CC1(C)CCc2ncnc(N3CCOc4ccc(-c5cnc(N)c(S(=O)(=O)N6CCC(O)CC6)c5)cc4C3)c2C1] | 1-({2-amino-5-[4-(6,6-dimethyl- 5,6,7,8-tetrahydroquinazolin-4- yl)-2,3,4,5-tetrahydro-1,4- benzoxazepin-7-yl]pyridin-3- yl}sulfonyl)piperidin-4-ol |
| 318 | 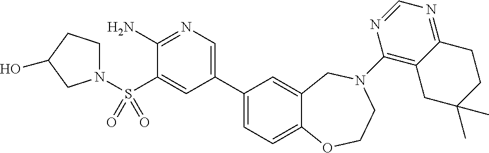 [SMILES: CC1(C)CCc2ncnc(N3CCOc4ccc(-c5cnc(N)c(S(=O)(=O)N6CCC(O)C6)c5)cc4C3)c2C1] | 1-({2-amino-5-[4-(6,6-dimethyl- 5,6,7,8-tetrahydroquinazolin-4- yl)-2,3,4,5-tetrahydro-1,4- benzoxazepin-7-yl]pyridin-3- yl}sulfonyl)piperidin-3-ol |
| 319 | 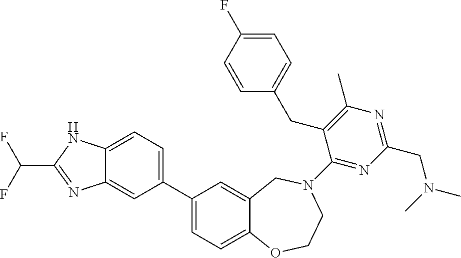 [SMILES: Cc1nc(CN(C)C)nc(N2CCOc3ccc(-c4ccc5[nH]c(C(F)F)nc5c4)cc3C2)c1Cc1ccc(F)cc1] | 1-(4-{7-[2-(difluoromethyl)-1H- benzimidazol-5-yl]-2,3-dihydro- 1,4-benzoxazepin-4(5H)-yl}-5- [(4-fluorophenyl)methyl]-6- methylpyrimidin-2-yl)-N,N- dimethylmethanamine |
| 320 | 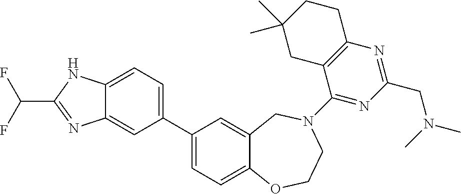 [SMILES: CN(C)Cc1nc2c(c(N3CCOc4ccc(-c5ccc6[nH]c(C(F)F)nc6c5)cc4C3)n1)CC(C)(C)CC2] | 1-(4-{7-[2-(difluoromethyl)-1H- benzimidazol-5-yl]-2,3-dihydro- 1,4-benzoxazepin-4(5H)-yl}-6,6- dimethyl-5,6,7,8- tetrahydroquinazolin-2-yl)-N,N- dimethylmethanamine |
| 321 | 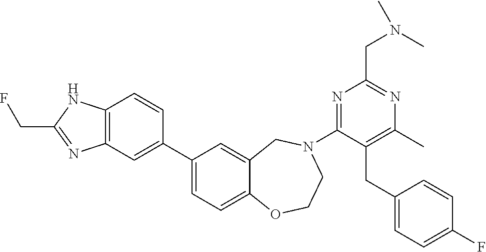 [SMILES: Cc1nc(CN(C)C)nc(N2CCOc3ccc(-c4ccc5[nH]c(CF)nc5c4)cc3C2)c1Cc1ccc(F)cc1] | 1-(4-{7-[2-(fluoromethyl)-1H- benzimidazol-5-yl]-2,3-dihydro- 1,4-benzoxazepin-4(5H)-yl}-5- [(4-fluorophenyl)methyl]-6- methylpyrimidin-2-yl)-N,N- dimethylmethanamine |
| 322 | 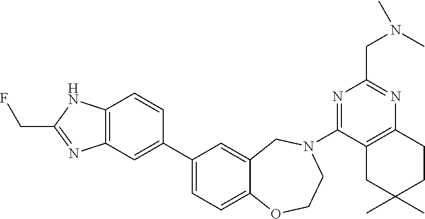 [SMILES: CN(C)Cc1nc2c(c(N3CCOc4ccc(-c5ccc6[nH]c(CF)nc6c5)cc4C3)n1)CC(C)(C)CC2] | 1-(4-{7-[2-(fluoromethyl)-1H- benzimidazol-5-yl]-2,3-dihydro- 1,4-benzoxazepin-4(5H)-yl}-6,6- dimethyl-5,6,7,8- tetrahydroquinazolin-2-yl)-N,N- dimethylmethanamine |
| 323 | 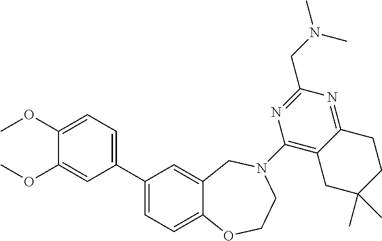 | 1-(4-{7-[3,4- bis(methyloxy)phenyl]-2,3- dihydro-1,4-benzoxazepin- 4(5H)-yl}-6,6-dimethyl-5,6,7,8- tetrahydroquinazolin-2-yl)-N,N- dimethylmethanamine |
| 324 | 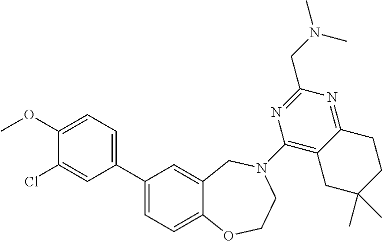 | 1-(4-{7-[3-chloro-4- (methyloxy)phenyl]-2,3- dihydro-1,4-benzoxazepin- 4(5H)-yl}-6,6-dimethyl-5,6,7,8- tetrahydroquinazolin-2-yl)-N,N- dimethylmethanamine |
| 325 | 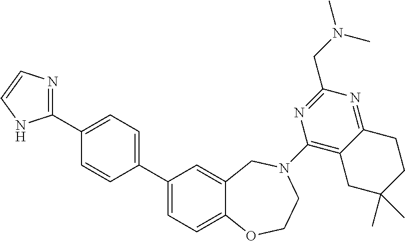 [SMILES: CN(C)Cc1nc2c(c(N3CCOc4ccc(-c5ccc(-c6ncc[nH]6)cc5)cc4C3)n1)CC(C)(C)CC2] | 1-(4-{7-[4-(1H-imidazol-2- yl)phenyl]-2,3-dihydro-1,4- benzoxazepin-4(5H)-yl}-6,6- dimethyl-5,6,7,8- tetrahydroquinazolin-2-yl)-N,N- dimethylmethanamine |
| 326 | 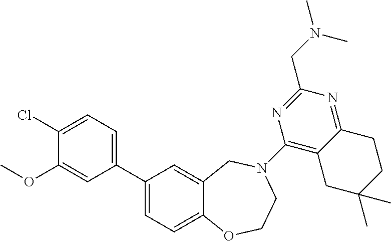 | 1-(4-{7-[4-chloro-3- (methyloxy)phenyl]-2,3- dihydro-1,4-benzoxazepin- 4(5H)-yl}-6,6-dimethyl-5,6,7,8- tetrahydroquinazolin-2-yl)-N,N- dimethylmethanamine |
| 327 | 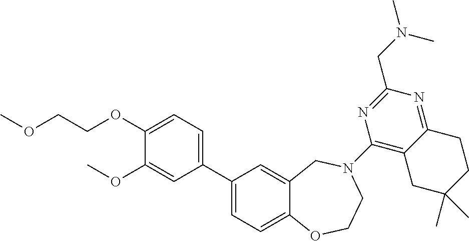 | 1-(6,6-dimethyl-4-{7-[3- (methyloxy)-4-{[2- (methyloxy)ethyl]oxy}phenyl]- 2,3-dihydro-1,4-benzoxazepin- 4(5H)-yl}-5,6,7,8- tetrahydroquinazolin-2-yl)-N,N- dimethylmethanamine |
| 328 | 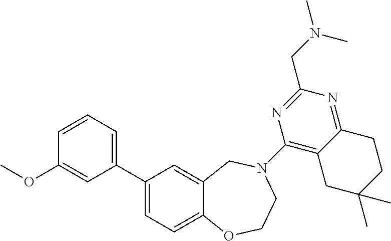 | 1-(6,6-dimethyl-4-{7-[3- (methyloxy)phenyl]-2,3- dihydro-1,4-benzoxazepin- 4(5H)-yl}-5,6,7,8- tetrahydroquinazolin-2-yl)-N,N- dimethylmethanamine |
| 329 | 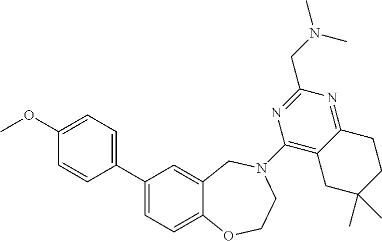 | 1-(6,6-dimethyl-4-{7-[4- (methyloxy)phenyl]-2,3- dihydro-1,4-benzoxazepin- 4(5H)-yl}-5,6,7,8- tetrahydroquinazolin-2-yl)-N,N- dimethylmethanamine |
| 330 | 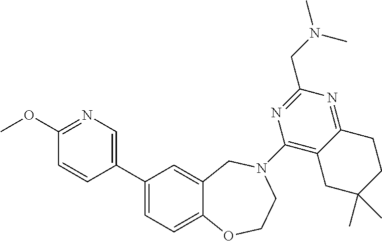 | 1-(6,6-dimethyl-4-{7-[6- (methyloxy)pyridin-3-yl]-2,3- dihydro-1,4-benzoxazepin- 4(5H)-yl}-5,6,7,8- tetrahydroquinazolin-2-yl)-N,N- dimethylmethanamine |
| 331 | 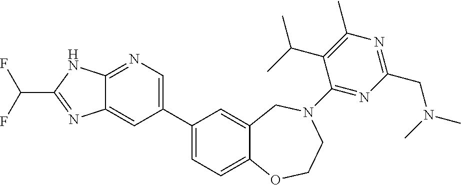 [SMILES: Cc1nc(CN(C)C)nc(N2CCOc3ccc(-c4cnc5[nH]c(C(F)F)nc5c4)cc3C2)c1C(C)C] | 1-[4-{7-[2-(difluoromethyl)-1H- benzimidazol-5-yl]-2,3-dihydro- 1,4-benzoxazepin-4(5H)-yl}-6- methyl-5-(1- methylethyl)pyrimidin-2-yl]- N,N-dimethylmethanamine |
| 332 | 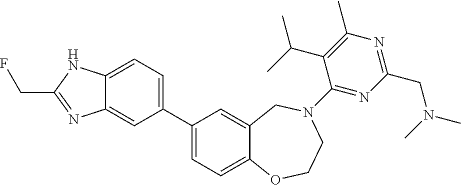 [SMILES: Cc1nc(CN(C)C)nc(N2CCOc3ccc(-c4ccc5[nH]c(CF)nc5c4)cc3C2)c1C(C)C] | 1-[4-{7-[2-(fluoromethyl)-1H- benzimidazol-5-yl]-2,3-dihydro- 1,4-benzoxazepin-4(5H)-yl}-6- methyl-5-(1- methylethyl)pyrimidin-2-yl]- N,N-dimethylmethanamine |
| 333 | 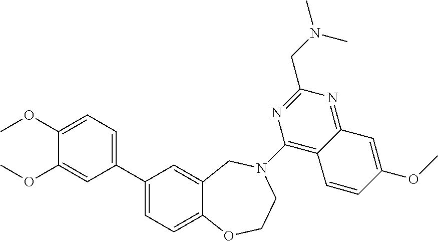 | 1-[4-{7-[3,4- bis(methyloxy)phenyl]-2,3- dihydro-1,4-benzoxazepin- 4(5H)-yl}-7- (methyloxy)quinazolin-2-yl]- N,N-dimethylmethanamine |
| 334 | 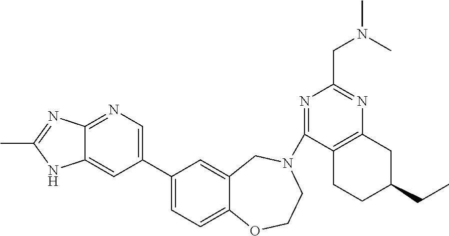 [SMILES: CC[C@H]1CCc2c(nc(CN(C)C)nc2N2CCOc3ccc(-c4cnc5nc(C)[nH]c5c4)cc3C2)C1] | 1-{(7S)-7-ethyl-4-[7-(2-methyl- 1H-imidazo[4,5-b]pyridin-6-yl)- 2,3-dihydro-1,4-benzoxazepin- 4(5H)-yl]-5,6,7,8- tetrahydroquinazolin-2-yl}-N,N- dimethylmethanamine |
| 335 | 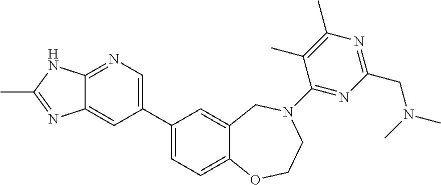 | 1-{4,5-dimethyl-6-[7-(2-methyl- 3H-imidazo[4,5-b]pyridin-6-yl)- 2,3-dihydro-1,4-benzoxazepin- 4(5H)-yl]pyrimidin-2-yl}-N,N- dimethylmethanamine |
| 336 | 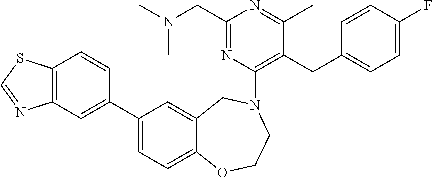 [SMILES: Cc1nc(CN(C)C)nc(N2CCOc3ccc(-c4ccc5scnc5c4)cc3C2)c1Cc1ccc(F)cc1] | 1-{4-[7-(1,3-benzothiazol-5-yl)- 2,3-dihydro-1,4-benzoxazepin- 4(5H)-yl]-5-[(4- fluorophenyl)methyl]-6- methylpyrimidin-2-yl}-N,N- dimethylmethanamine |
| 337 | 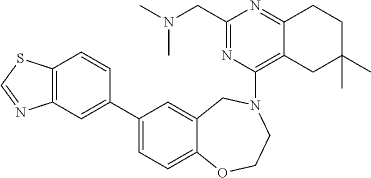 [SMILES: CN(C)Cc1nc2c(c(N3CCOc4ccc(-c5ccc6scnc6c5)cc4C3)n1)CC(C)(C)CC2] | 1-{4-[7-(1,3-benzothiazol-5-yl)- 2,3-dihydro-1,4-benzoxazepin- 4(5H)-yl]-6,6-dimethyl-5,6,7,8- tetrahydroquinazolin-2-yl}-N,N- dimethylmethanamine |
| 338 | 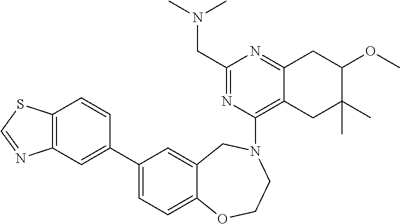 [SMILES: COC1Cc2nc(CN(C)C)nc(N3CCOc4ccc(-c5ccc6scnc6c5)cc4C3)c2CC1(C)C] | 1-{4-[7-(1,3-benzothiazol-5-yl)- 2,3-dihydro-1,4-benzoxazepin- 4(5H)-yl]-6,6-dimethyl-7- (methyloxy)-5,6,7,8- tetrahydroquinazolin-2-yl}-N,N- dimethylmethanamine |
| 339 | 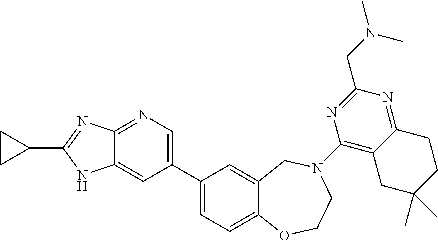 [SMILES: CN(C)Cc1nc2c(c(N3CCOc4ccc(-c5cnc6nc(C7CC7)[nH]c6c5)cc4C3)n1)CC(C)(C)CC2] | 1-{4-[7-(2-cyclopropyl-1H- imidazo[4,5-b]pyridin-6-yl)-2,3- dihydro-1,4-benzoxazepin- 4(5H)-yl]-6,6-dimethyl-5,6,7,8- tetrahydroquinazolin-2-yl}-N,N- dimethylmethanamine |
| 340 | 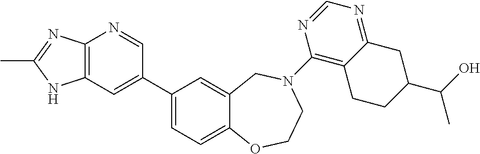 [SMILES: Cc1nc2ncc(-c3ccc4c(c3)CN(c3ncnc5c3CCC(C(C)O)C5)CCO4)cc2[nH]1] | 1-{4-[7-(2-methyl-1H- imidazo[4,5-b]pyridin-6-yl)-2,3- dihydro-1,4-benzoxazepin- 4(5H)-yl]-5,6,7,8- tetrahydroquinazolin-7- yl}ethanol |
| 341 | 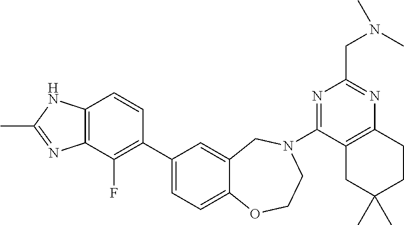 [SMILES: Cc1nc2c(F)c(-c3ccc4c(c3)CN(c3nc(CN(C)C)nc5c3CC(C)(C)CC5)CCO4)ccc2[nH]1] | 1-{4-[7-(4-fluoro-2-methyl-1H- benzimidazol-5-yl)-2,3-dihydro- 1,4-benzoxazepin-4(5H)-yl]-6,6- dimethyl-5,6,7,8- tetrahydroquinazolin-2-yl}-N,N- dimethylmethanamine |
| 342 | 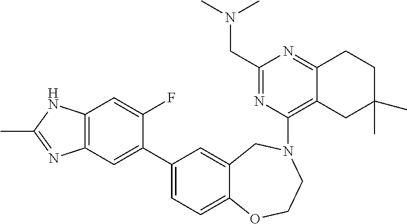 | 1-{4-[7-(6-fluoro-2-methyl-1H- benzimidazol-5-yl)-2,3-dihydro- 1,4-benzoxazepin-4(5H)-yl]-6,6- dimethyl-5,6,7,8- tetrahydroquinazolin-2-yl}-N,N- dimethylmethanamine |
| 343 | 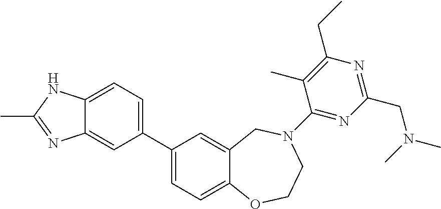 | 1-{4-ethyl-5-methyl-6-[7-(2- methyl-1H-benzimidazol-5-yl)- 2,3-dihydro-1,4-benzoxazepin- 4(5H)-yl]pyrimidin-2-yl}-N,N- dimethylmethanamine |
| 344 | 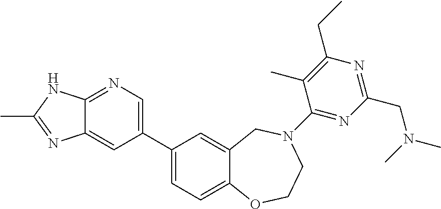 | 1-{4-ethyl-5-methyl-6-[7-(2- methyl-3H-imidazo[4,5- b]pyridin-6-yl)-2,3-dihydro-1,4- benzoxazepin-4(5H)- yl]pyrimidin-2-yl}-N,N- dimethylmethanamine |
| 345 | 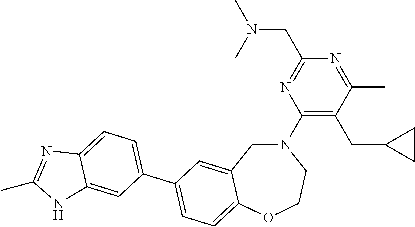 [SMILES: Cc1nc2ccc(-c3ccc4c(c3)CN(c3nc(CN(C)C)nc(C)c3CC3CC3)CCO4)cc2[nH]1] | 1-{5-(cyclopropylmethyl)-4- methyl-6-[7-(2-methyl-1H- benzimidazol-6-yl)-2,3-dihydro- 1,4-benzoxazepin-4(5H)- yl]pyrimidin-2-yl}-N,N- dimethylmethanamine |
| 346 | 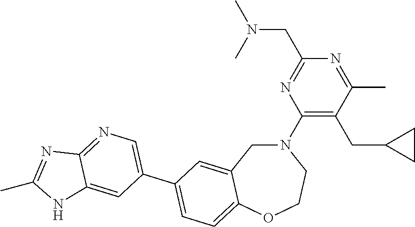 [SMILES: Cc1nc2ncc(-c3ccc4c(c3)CN(c3nc(CN(C)C)nc(C)c3CC3CC3)CCO4)cc2[nH]1] | 1-{5-(cyclopropylmethyl)-4- methyl-6-[7-(2-methyl-1H- imidazo[4,5-b]pyridin-6-yl)-2,3- dihydro-1,4-benzoxazepin- 4(5H)-yl]pyrimidin-2-yl}-N,N- dimethylmethanamine |
| 347 | 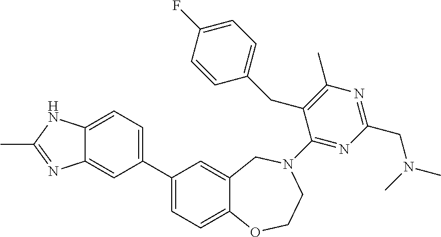 | 1-{5-[(4-fluorophenyl)methyl]- 4-methyl-6-[7-(2-methyl-1H- benzimidazol-5-yl)-2,3-dihydro- 1,4-benzoxazepin-4(5H)- yl]pyrimidin-2-yl}-N,N- dimethylmethanamine |
| 348 | 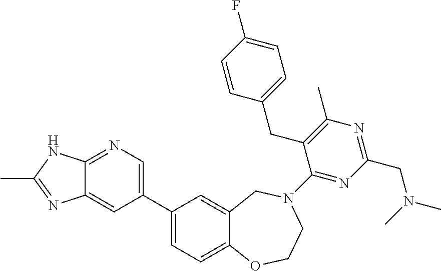 | 1-{5-[(4-fluorophenyl)methyl]- 4-methyl-6-[7-(2-methyl-3H- imidazo[4,5-b]pyridin-6-yl)-2,3- dihydro-1,4-benzoxazepin- 4(5H)-yl]pyrimidin-2-yl}-N,N- dimethylmethanamine |
| 349 | 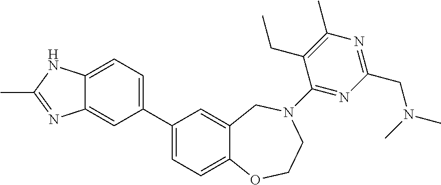 | 1-{5-ethyl-4-methyl-6-[7-(2- methyl-1H-benzimidazol-5-yl)- 2,3-dihydro-1,4-benzoxazepin- 4(5H)-yl]pyrimidin-2-yl}-N,N- dimethylmethanamine |
| 350 | 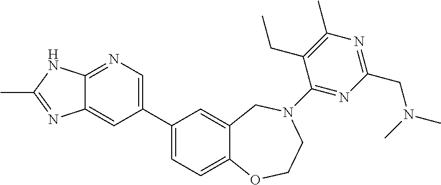 | 1-{5-ethyl-4-methyl-6-[7-(2- methyl-3H-imidazo[4,5- b]pyridin-6-yl)-2,3-dihydro-1,4- benzoxazepin-4(5H)- yl]pyrimidin-2-yl}-N,N- dimethylmethanamine |
| 351 | 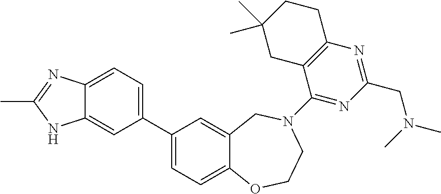 | 1-{6,6-dimethyl-4-[7-(2-methyl- 1H-benzimidazol-6-yl)-2,3- dihydro-1,4-benzoxazepin- 4(5H)-yl]-5,6,7,8- tetrahydroquinazolin-2-yl}-N,N- dimethylmethanamine |
| 352 | 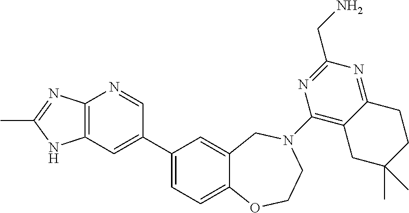 | 1-{6,6-dimethyl-4-[7-(2-methyl- 1H-imidazo[4,5-b]pyridin-6-yl)- 2,3-dihydro-1,4-benzoxazepin- 4(5H)-yl]-5,6,7,8- tetrahydroquinazolin-2- yl}methanamine |
| 353 | 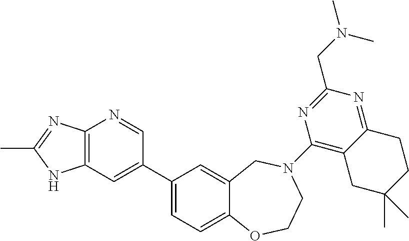 | 1-{6,6-dimethyl-4-[7-(2-methyl- 1H-imidazo[4,5-b]pyridin-6-yl)- 2,3-dihydro-1,4-benzoxazepin- 4(5H)-yl]-5,6,7,8- tetrahydroquinazolin-2-yl}-N,N- dimethylmethanamine |
| 354 | 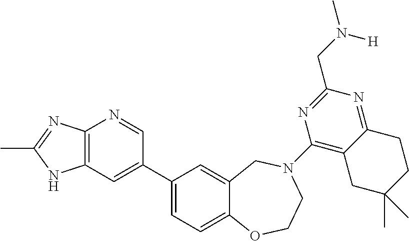 | 1-{6,6-dimethyl-4-[7-(2-methyl- 1H-imidazo[4,5-b]pyridin-6-yl)- 2,3-dihydro-1,4-benzoxazepin- 4(5H)-yl]-5,6,7,8- tetrahydroquinazolin-2-yl}-N- methylmethanamine |
| 355 | 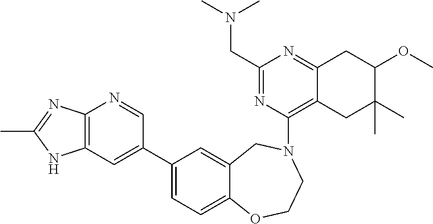 | 1-{6,6-dimethyl-4-[7-(2-methyl- 1H-imidazo(4,5-b]pyridin-6-yl)- 2,3-dihydro-1,4-benzoxazepin- 4(5H)-yl]-7-(methyloxy)- 5,6,7,8-tetrahydroquinazolin-2- yl}-N,N-dimethylmethanamine |
| 356 | 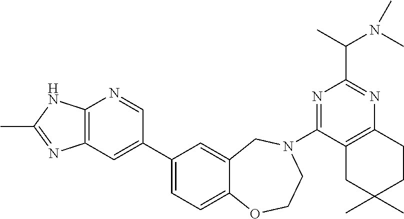 | l-{6,6-dimethyl-4-[7-(2-methyl- 3H-imidazo[4,5-b]pyridin-6-yl)- 2,3-dihydro-1,4-benzoxazepin- 4(5H)-yl]-5,6,7,8- tetrahydroquinazolin-2-yl}-N,N- dimethylethanamine |
| 357 | 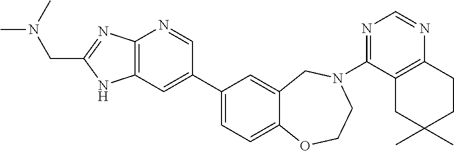 | 1-{6-[4-(6,6-dimethyl-5,6,7,8- tetrahydroquinazolin-4-yl)- 2,3,4,5-tetrahydro-1,4- benzoxazepin-7-yl]-1H- imidazo[4,5-b]pyridin-2-yl}- N,N-dimethylmethanamine |
| 358 | 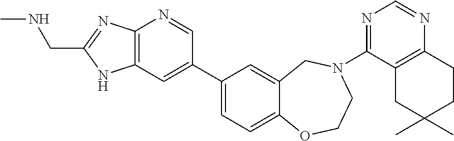 | 1-{6-[4-(6,6-dimethyl-5,6,7,8- tetrahydroquinazolin-4-yl)- 2,3,4,5-tetrahydro-1,4- benzoxazepin-7-yl]-1H- imidazo[4,5-b]pyridin-2-yl}-N- methylmethanamine |
| 359 | 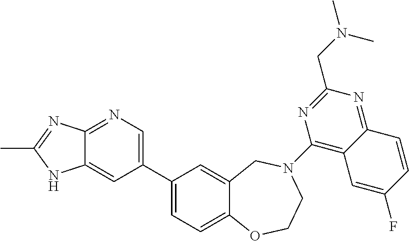 [SMILES: Cc1nc2ncc(-c3ccc4c(c3)CN(c3nc(CN(C)C)nc5ccc(F)cc35)CCO4)cc2[nH]1] | 1-{6-fluoro-4-[7-(2-methyl-1H- imidazo[4,5-b]pyridin-6-yl)-2,3- dihydro-1,4-benzoxazepin- 4(5H)-yl]quinazolin-2-yl}-N,N- dimethylmethanamine |
| 360 | 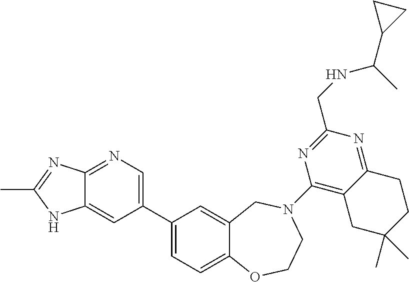 | 1-cyclopropyl-N-({6,6-dimethyl- 4-[7-(2-methyl-1H-imidazo[4,5- b]pyridin-6-yl)-2,3-dihydro-1,4- benzoxazepin-4(5H)-yl]-5,6,7,8- tetrahydroquinazolin-2- yl}methyl)ethanamine |
| 361 | 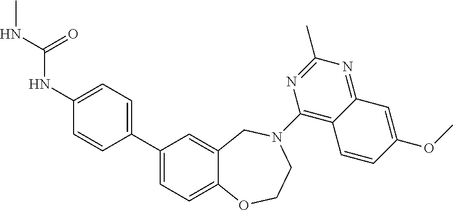 [SMILES: CNC(=O)Nc1ccc(-c2ccc3c(c2)CN(c2nc(C)nc4cc(OC)ccc24)CCO3)cc1] | 1-methyl-3-(4-{4-[2-methyl-7- (methyloxy)quinazolin-4-yl]- 2,3,4,5-tetrahydro-1,4- benzoxazepin-7-yl}phenyl)urea |
| 362 | 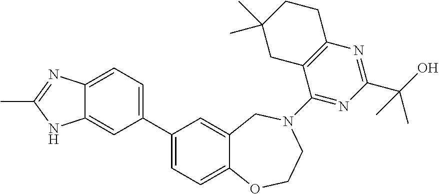 [SMILES: Cc1nc2ccc(-c3ccc4c(c3)CN(c3nc(C(C)(C)O)nc5c3CC(C)(C)CC5)CCO4)cc2[nH]1] | 2-{6,6-dimethyl-4-[7-(2-methyl- 1H-benzimidazol-6-yl)-2,3- dihydro-1,4-benzoxazepin- 4(5H)-yl]-5,6,7,8- tetrahydroquinazolin-2- yl}propan-2-ol |
| 363 | 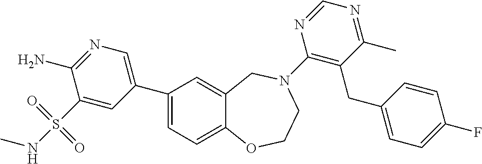 [SMILES: CNS(=O)(=O)c1cc(-c2ccc3c(c2)CN(c2ncnc(C)c2Cc2ccc(F)cc2)CCO3)cnc1N] | 2-amino-5-(4-{5-[(4- fluorophenyl)methyl]-6- methylpyrimidin-4-yl)-2,3,4,5- tetrahydro-1,4-benzoxazepin-7- yl)-N-methylpyridine-3- sulfonamide |
| 364 | 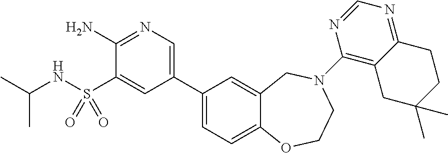 [SMILES: CC(C)NS(=O)(=O)c1cc(-c2ccc3c(c2)CN(c2ncnc4c2CC(C)(C)CC4)CCO3)cnc1N] | 2-amino-5-[4-(6,6-dimethyl- 5,6,7,8-tetrahydroquinazolin-4- yl)-2,3,4,5-tetrahydro-1,4- benzoxazepin-7-yl]-N-(1- methylethyl)pyridine-3- sulfonamide |
| 365 | 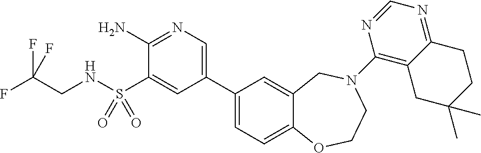 [SMILES: CC1(C)CCc2ncnc(N3CCOc4ccc(-c5cnc(N)c(S(=O)(=O)NCC(F)(F)F)c5)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]-N-(2,2,2- trifluoroethyl)pyridine-3- sulfonamide |
| 366 | 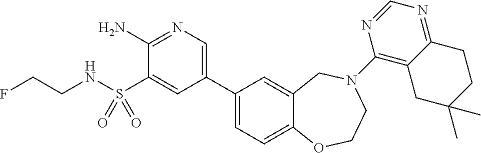 [SMILES: CC1(C)CCc2ncnc(N3CCOc4ccc(-c5cnc(N)c(S(=O)(=O)NCCF)c5)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]-N-(2- fluoroethyl)pyridine-3- sulfonamide |
| 367 | 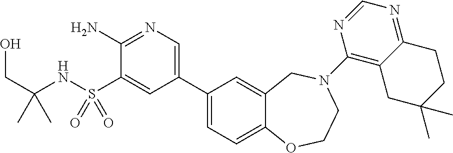 [SMILES: CC1(C)CCc2ncnc(N3CCOc4ccc(-c5cnc(N)c(S(=O)(=O)NC(C)(C)CO)c5)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]-N-(2- hydroxy-1,1- dimethylethyl)pyridine-3- sulfonamide |
| 368 | 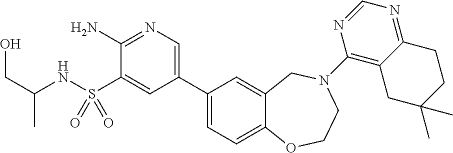 [SMILES: CC(CO)NS(=O)(=O)c1cc(-c2ccc3c(c2)CN(c2ncnc4c2CC(C)(C)CC4)CCO3)cnc1N] | 2-amino-5-[4-(6,6-dimethyl- 5,6,7,8-tetrahydroquinazolin-4- yl)-2,3,4,5-tetrahydro-1,4- benzoxazepin-7-yl]-N-(2- hydroxy-1-methylethyl)pyridine- 3-sulfonamide |
| 369 | 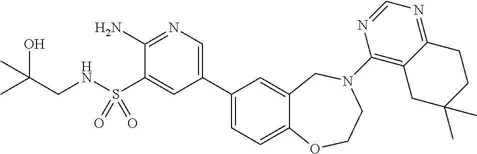 [SMILES: CC(C)(O)CNS(=O)(=O)c1cc(-c2ccc3c(c2)CN(c2ncnc4c2CC(C)(C)CC4)CCO3)cnc1N] | 2-amino-5-[4-(6,6-dimethyl- 5,6,7,8-tetrahydroquinazolin-4- yl)-2,3,4,5-tetrahydro-1,4- benzoxazepin-7-yl]-N-(2- hydroxy-2- methylpropyl)pyridine-3- sulfonamide |
| 370 | 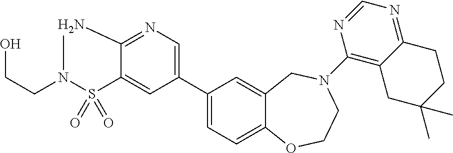 [SMILES: CC1(C)CCc2ncnc(N3CCOc4ccc(-c5cnc6c(c5)S(=O)(=O)N(CCO)N6)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]-N-(2- hydroxyethyl)-N- methylpyridine-3-sulfonamide |
| 371 | 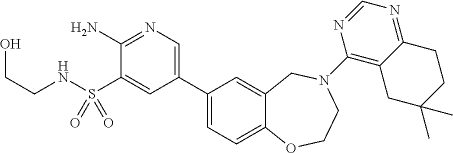 [SMILES: CC1(C)CCc2ncnc(N3CCOc4ccc(-c5cnc(N)c(S(=O)(=O)NCCO)c5)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]-N-(2- hydroxyethyl)pyridine-3- sulfonamide |
| 372 | 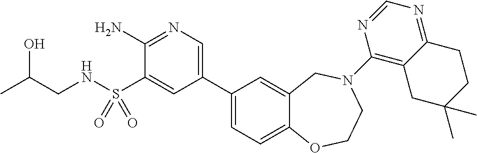 [SMILES: CC(O)CNS(=O)(=O)c1cc(-c2ccc3c(c2)CN(c2ncnc4c2CC(C)(C)CC4)CCO3)cnc1N] | 2-amino-5-[4-(6,6-dimethyl- 5,6,7,8-tetrahydroquinazolin-4- yl)-2,3,4,5-tetrahydro-1,4- benzoxazepin-7-yl]-N-(2- hydroxypropyl)pyridine-3- sulfonamide |
| 373 | 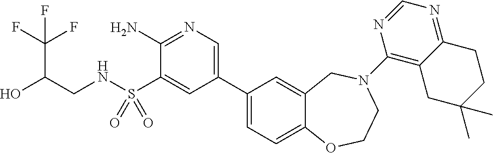 [SMILES: CC1(C)CCc2ncnc(N3CCOc4ccc(-c5cnc(N)c(S(=O)(=O)NCC(O)C(F)(F)F)c5)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]-N-(3,3,3- trifluoro-2- hydroxypropyl)pyridine-3- sulfonamide |
| 374 | 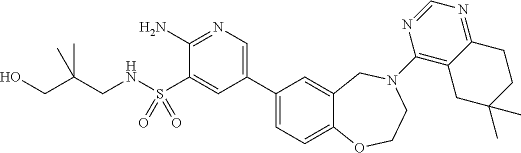 [SMILES: CC(C)(CO)CNS(=O)(=O)c1cc(-c2ccc3c(c2)CN(c2ncnc4c2CC(C)(C)CC4)CCO3)cnc1N] | 2-amino-5-[4-(6,6-dimethyl- 5,6,7,8-tetrahydroquinazolin-4- yl)-2,3,4,5-tetrahydro-1,4- benzoxazepin-7-yl]-N-(3- hydroxy-2,2- dimethylpropyl)pyridine-3- sulfonamide |
| 375 | 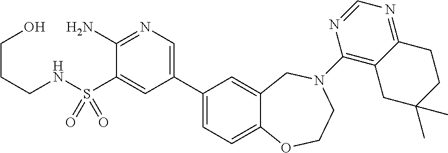 [SMILES: CC1(C)CCc2ncnc(N3CCOc4ccc(-c5cnc(N)c(S(=O)(=O)NCCCO)c5)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]-N-(3- hydroxypropyl)pyridine-3- sulfonamide |
| 376 | 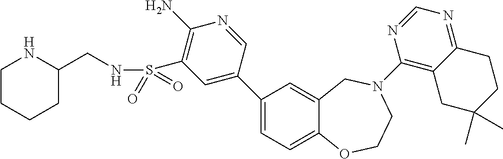 [SMILES: CC1(C)CCc2ncnc(N3CCOc4ccc(-c5cnc(N)c(S(=O)(=O)NCC6CCCCN6)c5)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]-N- (piperidin-2-ylmethyl)pyridine- 3-sulfonamide |
| 377 | 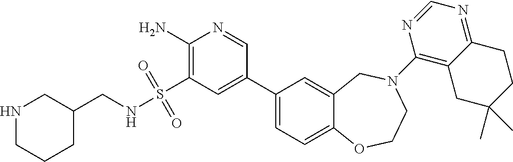 [SMILES: CC1(C)CCc2ncnc(N3CCOc4ccc(-c5cnc(N)c(S(=O)(=O)NCC6CCCNC6)c5)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]-N- (piperidin-3-ylmethyl)pyridine- 3-sulfonamide |
| 378 | 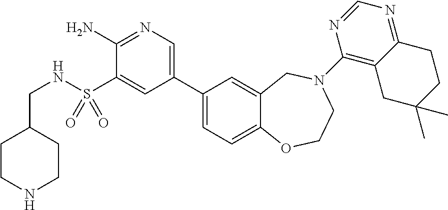 [SMILES: CC1(C)CCc2ncnc(N3CCOc4ccc(-c5cnc(N)c(S(=O)(=O)NCC6CCNCC6)c5)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]-N- (piperidin-4-ylmethyl)pyridine- 3-sulfonamide |
| 379 | 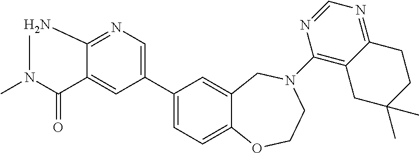 | 2-amino-5-(4-(6,6-dimethyl- 5,6,7,8-tetrahydroquinazolin-4- yl)-2,3,4,5-tetrahydro-1,4- benzoxazepin-7-yl]-N,N- dimethylpyridine-3-carboxamide |
| 380 | 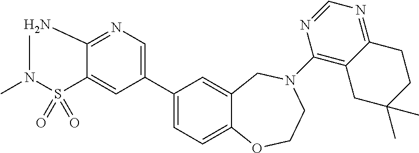 | 2-amino-5-(4-(6,6-dimethyl- 5,6,7,8-tetrahydroquinazolin-4- yl)-2,3,4,5-tetrahydro-1,4- benzoxazepin-7-yl]-N,N- dimethylpyridine-3-sulfonamide |
| 381 | 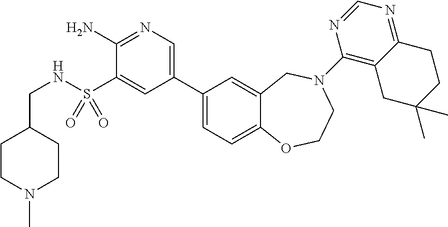 [SMILES: CN1CCC(CNS(=O)(=O)c2cc(-c3ccc4c(c3)CN(c3ncnc5c3CC(C)(C)CC5)CCO4)cnc2N)CC1] | 2-amino-5-[4-(6,6-dimethyl- 5,6,7,8-tetrahydroquinazolin-4- yl)-2,3,4,5-tetrahydro-1,4- benzoxazepin-7-yl]-N-[(1- methylpiperidin-4- yl)methyl]pyridine-3- sulfonamide |
| 382 | 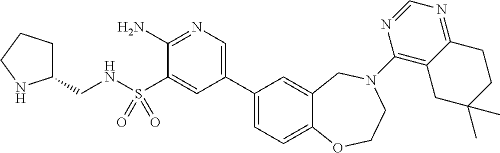 [SMILES: CC1(C)CCc2ncnc(N3CCOc4ccc(-c5cnc(N)c(S(=O)(=O)NC[C@H]6CCCN6)c5)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]-N-[(2R)- pyrrolidin-2-ylmethyl] pyridine- 3-sulfonamide |
| 383 | 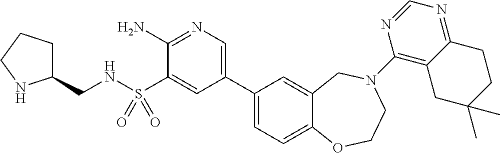 [SMILES: CC1(C)CCc2ncnc(N3CCOc4ccc(-c5cnc(N)c(S(=O)(=O)NC[C@@H]6CCCN6)c5)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]-N-[(2S)- pyrrolidin-2-ylmethyl]pyridine- 3-sulfonamide |
| 384 | 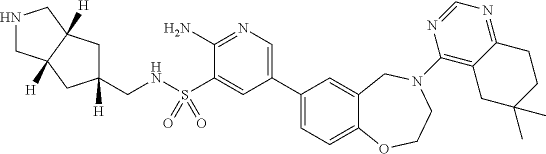 [SMILES: CC1(C)CCc2ncnc(N3CCOc4ccc(-c5cnc(N)c(S(=O)(=O)NC[C@H]6C[C@H]7CNC[C@H]7C6)c5)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]-N- [(3aR,5r,6aS)- octahydrocyclopenta[c]pyrrol-5- ylmethyl]pyridine-3- sulfonamide |
| 385 | 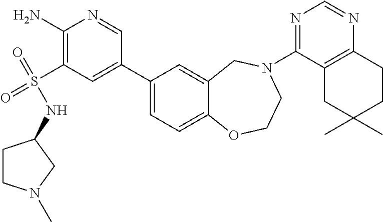 [SMILES: CN1CC[C@@H](NS(=O)(=O)c2cc(-c3ccc4c(c3)CN(c3ncnc5c3CC(C)(C)CC5)CCO4)cnc2N)C1] | 2-amino-5-[4-(6,6-dimethyl- 5,6,7,8-tetrahydroquinazolin-4- yl)-2,3,4,5-tetrahydro-1,4- benzoxazepin-7-yl]-N-[(3R)-1- methylpyrrolidin-3-yl]pyridine- 3-sulfonamide |
| 386 | 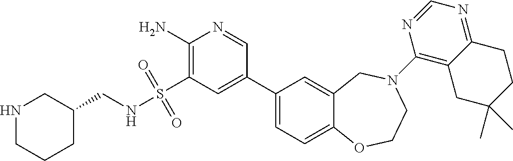 [SMILES: CC1(C)CCc2ncnc(N3CCOc4ccc(-c5cnc(N)c(S(=O)(=O)NC[C@@H]6CCCNC6)c5)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]-N-[(3R)- piperidin-3-ylmethyl]pyridine-3- sulfonamide |
| 387 | 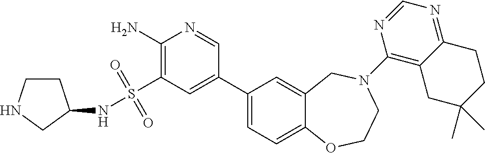 [SMILES: CC1(C)CCc2ncnc(N3CCOc4ccc(-c5cnc(N)c(S(=O)(=O)N[C@@H]6CCNC6)c5)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]-N-[(3R)- pyrrolidin-3-yl]pyridine-3- sulfonamide |
| 388 | 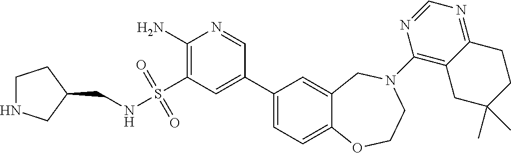 [SMILES: CC1(C)CCc2ncnc(N3CCOc4ccc(-c5cnc(N)c(S(=O)(=O)NC[C@@H]6CCNC6)c5)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]-N-[(3R)- pyrrolidin-3-ylmethyl]pyridine- 3-sulfonamide |
| 389 | 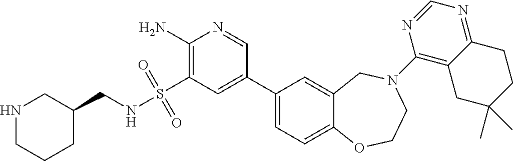 [SMILES: CC1(C)CCc2ncnc(N3CCOc4ccc(-c5cnc(N)c(S(=O)(=O)NC[C@H]6CCCNC6)c5)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]-N-[(3S)- pyrrolidin-3-ylmethyl]pyridine-3- sulfonamide |
| 390 | 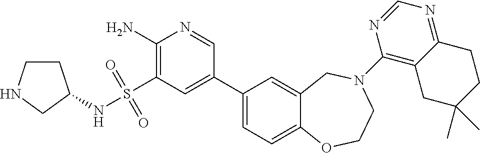 [SMILES: CC1(C)CCc2ncnc(N3CCOc4ccc(-c5cnc(N)c(S(=O)(=O)N[C@H]6CCNC6)c5)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]-N-[(3S)- pyrrolidin-3-yl]pyridine-3- sulfonamide |
| 391 | 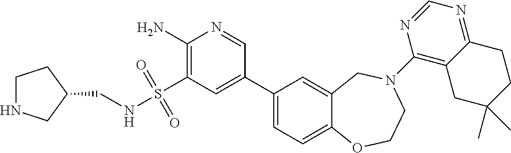 [SMILES: CC1(C)CCc2ncnc(N3CCOc4ccc(-c5cnc(N)c(S(=O)(=O)NC[C@H]6CCNC6)c5)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]-N-[(3S)- pyrrolidin-3-ylmethyl]pyridine- 3-sulfonamide |
| 392 | 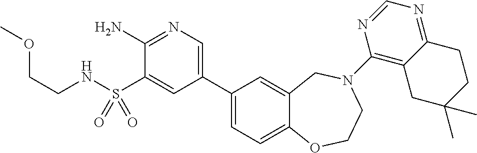 | 2-amino-5-[4-(6,6-dimethyl- 5,6,7,8-tetrahydroquinazolin-4- yl)-2,3,4,5-tetrahydro-1,4- benzoxazepin-7-yl]-N-[2- (methyloxy)ethyl]pyridine-3- sulfonamide |
| 393 | 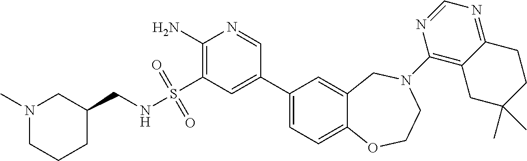 | 2-amino-5-[4-(6,6-dimethyl- 5,6,7,8-tetrahydroquinazolin-4- yl)-2,3,4,5-tetrahydro-1,4- benzoxazepin-7-yl]-N-{[(3S)-1- methylpiperidin-3- yl]methyl}pyridine-3- sulfonamide |
| 394 | 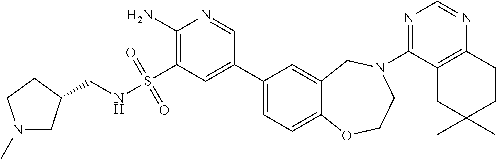 | 2-amino-5-[4-(6,6-dimethyl- 5,6,7,8-tetrahydroquinazolin-4- yl)-2,3,4,5-tetrahydro-1,4- benzoxazepin-7-yl]-N-{[(3S)-1- methylpyrrolidin-3- yl]methyl}pyridine-3- sulfonamide |
| 395 | 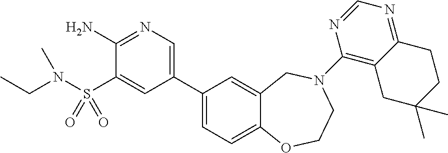 [SMILES: CCN(C)S(=O)(=O)c1cc(-c2ccc3c(c2)CN(c2ncnc4c2CC(C)(C)CC4)CCO3)cnc1N] | 2-amino-5-[4-(6,6-dimethyl- 5,6,7,8-tetrahydroquinazolin-4- yl)-2,3,4,5-tetrahydro-1,4- benzoxazepin-7-yl]-N-ethyl-N- methylpyridine-3-sulfonamide |
| 396 | 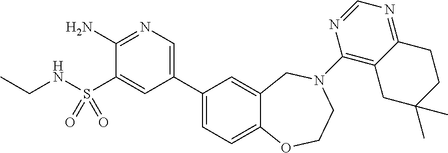 [SMILES: CCNS(=O)(=O)c1cc(-c2ccc3c(c2)CN(c2ncnc4c2CC(C)(C)CC4)CCO3)cnc1N] | 2-amino-5-[4-(6,6-dimethyl- 5,6,7,8-tetrahydroquinazolin-4- yl)-2,3,4,5-tetrahydro-1,4- benzoxazepin-7-yl]-N- ethylpyridine-3-sulfonamide |
| 397 | 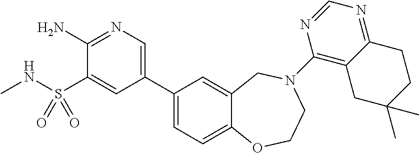 | 2-amino-5-{4-(6,6-dimethyl- 5,6,7,8-tetrahydroquinazolin-4- yl)-2,3,4,5-tetrahydro-1,4- benzoxazepin-7-yl]-N- methylpyridine-3-sulfonamide |
| 398 | 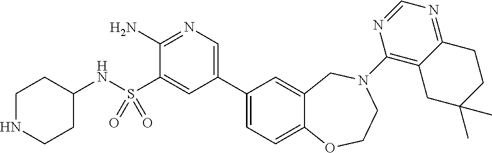 [SMILES: CC1(C)CCc2ncnc(N3CCOc4ccc(-c5cnc(N)c(S(=O)(=O)NC6CCNCC6)c5)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]-N-piperidin- 4-ylpyridine-3-sulfonamide |
| 399 | 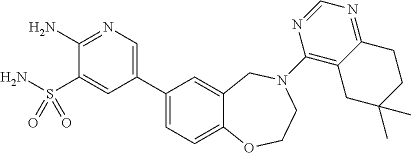 [SMILES: CC1(C)CCc2ncnc(N3CCOc4ccc(-c5cnc(N)c(S(N)(=O)=O)c5)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]pyridine-3- sulfonamide |
| 400 | 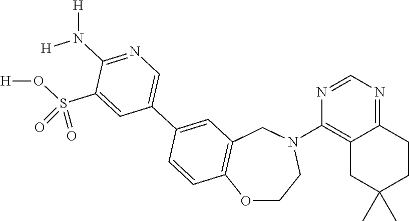 [SMILES: CC1(C)CCc2ncnc(N3CCOc4ccc(-c5cnc(N)c(S(=O)(=O)O)c5)cc4C3)c2C1] | 2-amino-5-[4-(6,6-dimethyl- 5,6,7,8-tetrahydroquinazolin-4- yl)-2,3,4,5-tetrahydro-1,4- benzoxazepin-7-yl]pyridine-3- sulfonic acid |
| 401 | 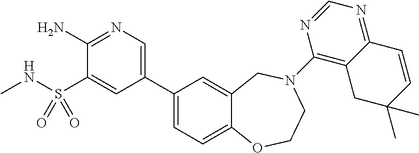 | 2-amino-5-[4-(6,6-dimethyl-5,6- dihydroquinazolin-4-yl)-2,3,4,5- tetrahydro-1,4-benzoxazepin-7- yl]-N-methylpyridine-3- sulfonamide |
| 402 | 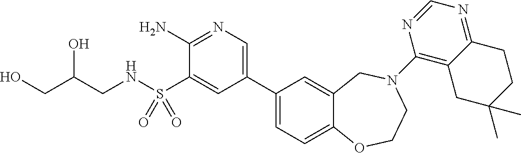 [SMILES: CC1(C)CCc2ncnc(N3CCOc4ccc(-c5cnc(N)c(S(=O)(=O)NCC(O)CO)c5)cc4C3)c2C1] | 2-amino-N-(2,3- dihydroxypropyl)-5-[4-(6,6- dimethyl-5,6,7,8- tetrahydroquinazolin-4-yl)- 2,3,4,5-tetrahydro-1,4- benzoxazepin-7-yl]pyridine-3- sulfonamide |
| 403 | 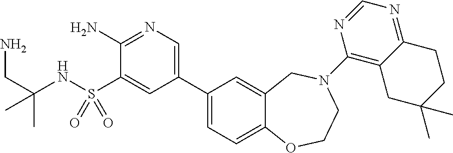 [SMILES: CC1(C)CCc2ncnc(N3CCOc4ccc(-c5cnc(N)c(S(=O)(=O)NC(C)(C)CN)c5)cc4C3)c2C1] | 2-amino-N-(2-amino-1,1- dimethylethyl)-5-[4-(6,6- dimethyl-5,6,7,8- tetrahydroquinazolin-4-yl)- 2,3,4,5-tetrahydro-1,4- benzoxazepin-7-yl]pyridine-3- sulfonamide |
| 405 | 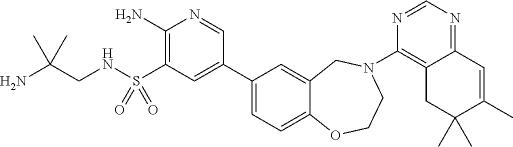 [SMILES: CC1=Cc2ncnc(N3CCOc4ccc(-c5cnc(N)c(S(=O)(=O)NCC(C)(C)N)c5)cc4C3)c2CC1(C)C] | 2-amino-N-(2-amino-2- methylpropyl)-5-[4-(6,6,7- trimethyl-5,6-dihydroquinazolin- 4-yl)-2,3,4,5-tetrahydro-1,4- benzoxazepin-7-yl]pyridine-3- sulfonamide |
| 406 | 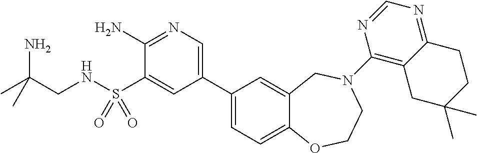 [SMILES: CC(C)(N)CNS(=O)(=O)c1cc(-c2ccc3c(c2)CN(c2ncnc4c2CC(C)(C)CC4)CCO3)cnc1N] | 2-amino-N-(2-amino-2- methylpropyl)-5-[4-(6,6- dimethyl-5,6,7,8- tetrahydroquinazolin-4-yl)- 2,3,4,5-tetrahydro-1,4- benzoxazepin-7-yl]pyridine-3- sulfonamide |
| 407 | 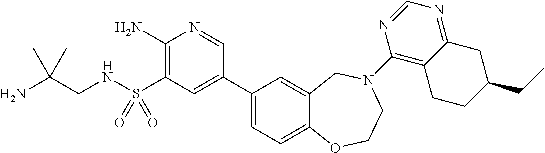 [SMILES: CC[C@H]1CCc2c(ncnc2N2CCOc3ccc(-c4cnc(N)c(S(=O)(=O)NCC(C)(C)N)c4)cc3C2)C1] | 2-amino-N-(2-amino-2- methylpropyl)-5-{4-[(7S)-7- ethyl-5,6,7,8- tetrahydroquinazolin-4-yl]- 2,3,4,5-tetrahydro-1,4- benzoxazepin-7-yl}pyridine-3- sulfonamide |
| 408 | 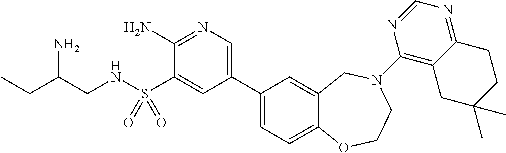 [SMILES: CCC(N)CNS(=O)(=O)c1cc(-c2ccc3c(c2)CN(c2ncnc4c2CC(C)(C)CC4)CCO3)cnc1N] | 2-amino-N-(2-aminobutyl)-5-[4- (6,6-dimethyl-5,6,7,8- tetrahydroquinazolin-4-yl)- 2,3,4,5-tetrahydro-1,4- benzoxazepin-7-yl]pyridine-3- sulfonamide |
| 409 | 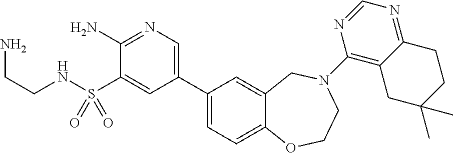 [SMILES: CC1(C)CCc2ncnc(N3CCOc4ccc(-c5cnc(N)c(S(=O)(=O)NCCN)c5)cc4C3)c2C1] | 2-amino-N-(2-aminoethyl)-5- [4-(6,6-dimethyl-5,6,7,8- tetrahydroquinazolin-4-yl)- 2,3,4,5-tetrahydro-1,4- benzoxazepin-7-yl]pyridine-3- sulfonamide |
| 410 | 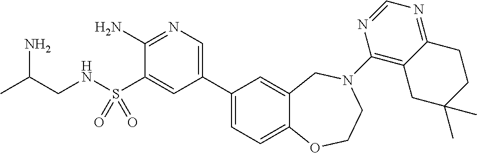 [SMILES: CC(N)CNS(=O)(=O)c1cc(-c2ccc3c(c2)CN(c2ncnc4c2CC(C)(C)CC4)CCO3)cnc1N] | 2-amino-N-(2-aminopropyl)-5- [4-(6,6-dimethyl-5,6,7,8- tetrahydroquinazolin-4-yl)- 2,3,4,5-tetrahydro-1,4- benzoxazepln-7-yl]pyridine-3- sulfonamide |
| 411 | 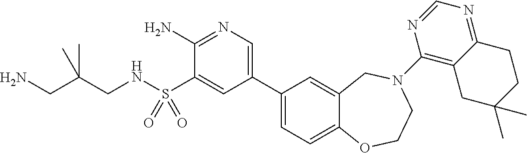 [SMILES: CC(C)(CN)CNS(=O)(=O)c1cc(-c2ccc3c(c2)CN(c2ncnc4c2CC(C)(C)CC4)CCO3)cnc1N] | 2-amino-N-(3-amino-2,2- dimethylpropyl)-5-[4-(6,6- dimethyl-5,6,7,8- tetrahydroquinazolin-4-yl)- 2,3,4,5-tetrahydro-1,4- benzoxazepin-7-yl]pyridine-3- sulfonamide |
| 412 | 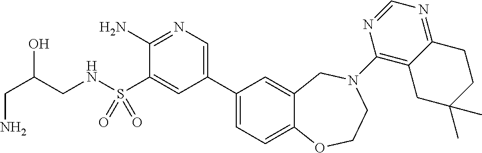 [SMILES: CC1(C)CCc2ncnc(N3CCOc4ccc(-c5cnc(N)c(S(=O)(=O)NCC(O)CN)c5)cc4C3)c2C1] | 2-amino-N-(3-amino-2- hydroxypropyl)-5-[4-(6,6- dimethyl-5,6,7,8- tetrahydroquinazolin-4-yl)- 2,3,4,5-tetrahydro-1,4- benzoxazepin-7-yl]pyridine-3- sulfonamide |
| 413 | 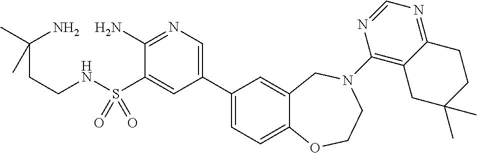 [SMILES: CC(C)(N)CCNS(=O)(=O)c1cc(-c2ccc3c(c2)CN(c2ncnc4c2CC(C)(C)CC4)CCO3)cnc1N] | 2-amino-N-(3-amino-3- methylbutyl)-5-[4-(6,6-dimethyl- 5,6,7,8-tetrahydroquinazolin-4- yl)-2,3,4,5-tetrahydro-1,4- benzoxazepin-7-yl]pyridine-3- sulfonamide |
| 414 | 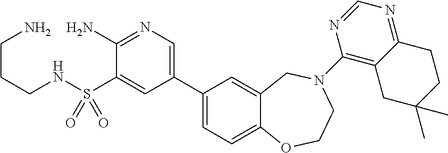 [SMILES: CC1(C)CCc2ncnc(N3CCOc4ccc(-c5cnc(N)c(S(=O)(=O)NCCCN)c5)cc4C3)c2C1] | 2-amino-N-(3-aminopropyl)-5- [4-(6,6-dimethyl-5,6,7,8- tetrahydroquinazolin-4-yl)- 2,3,4,5-tetrahydro-1,4- benzoxazepin-7-yl]pyridine-3- sulfonamide |
| 415 | 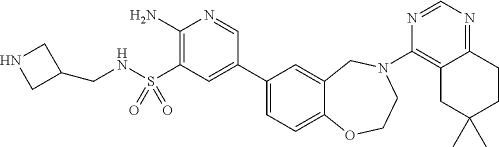 [SMILES: CC1(C)CCc2ncnc(N3CCOc4ccc(-c5cnc(N)c(S(=O)(=O)NCC6CNC6)c5)cc4C3)c2C1] | 2-amino-N-(azetidin-3- ylmethyl)-5-[4-(6,6-dimethyl- 5,6,7,8-tetrahydroquinazolin-4- yl)-2,3,4,5-tetrahydro-1,4- benzoxazepin-7-yl]pyridine-3- sulfonamide |
| 416 | 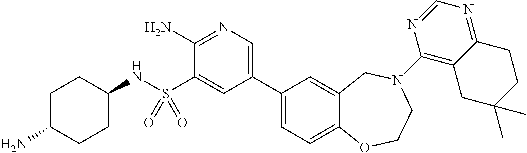 [SMILES: CC1(C)CCc2ncnc(N3CCOc4ccc(-c5cnc(N)c(S(=O)(=O)N[C@H]6CC[C@H](N)CC6)c5)cc4C3)c2C1] | 2-amino-N-(trans-4- aminocyclohexyl)-5-[4-(6,6- dimethyl-5,6,7,8- tetrahydroquinazolin-4-yl)- 2,3,4,5-tetrahydro-1,4- benzoxazepin-7-yl]pyridine-3- sulfonamide |
| 417 | 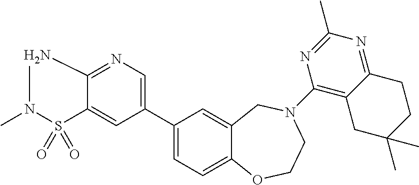 | 2-amino-N,N-dimethyl-5-[4- (2,6,6-trimethyl-5,6,7,8- tetrahydroquinazolin-4-yl)- 2,3,4,5-tetrahydro-1,4- benzoxazepin-7-yl]pyridine-3- sulfonamide |
| 418 | 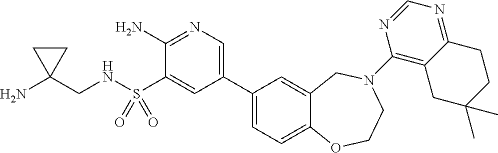 [SMILES: CC1(C)CCc2ncnc(N3CCOc4ccc(-c5cnc(N)c(S(=O)(=O)NCC6(N)CC6)c5)cc4C3)c2C1] | 2-amino-N-[(1- aminocyclopropyl)methyl]-5-[4- (6,6-dimethyl-5,6,7,8- tetrahydroquinazolin-4-yl)- 2,3,4,5-tetrahydro-1,4- benzoxazepin-7-yl]pyridine-3- sulfonamide |
| 419 | 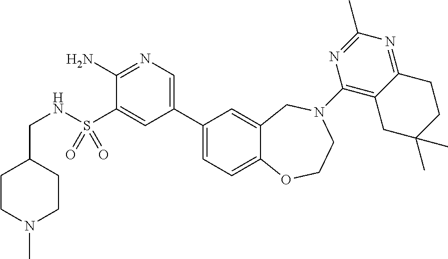 [SMILES: Cc1nc2c(c(N3CCOc4ccc(-c5cnc(N)c(S(=O)(=O)NCC6CCN(C)CC6)c5)cc4C3)n1)CC(C)(C)CC2] | 2-amino-N-[(1-methylpiperidin- 4-yl)methyl]-5-[4-(2,6,6- trimethyl-5,6,7,8- tetrahydroquinazolin-4-yl)- 2,3,4,5-tetrahydro-1,4- benzoxazepin-7-yl]pyridine-3- sulfonamide |
| 420 | 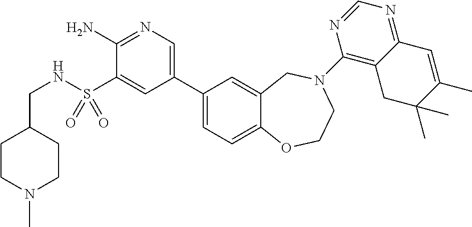 [SMILES: CC1=Cc2ncnc(N3CCOc4ccc(-c5cnc(N)c(S(=O)(=O)NCC6CCN(C)CC6)c5)cc4C3)c2CC1(C)C] | 2-amino-N-[(1-methylpiperidin- 4-yl)methyl]-5-[4-(6,6,7- trimethyl-5,6-dihydroquinazolin- 4-yl)-2,3,4,5-tetrahydro-1,4- benzoxazepin-7-yl]pyridine-3- sulfonamide |
| 421 | 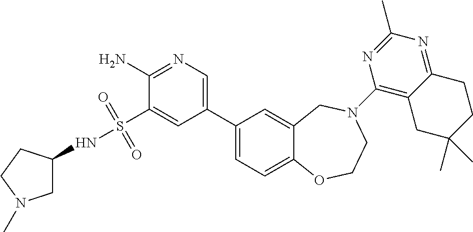 [SMILES: Cc1nc2c(c(N3CCOc4ccc(-c5cnc(N)c(S(=O)(=O)N[C@@H]6CCN(C)C6)c5)cc4C3)n1)CC(C)(C)CC2] | 2-amino-N-[(3R)-1- methylpyrrolidin-3-yl]-5-[4- (2,6,6-trimethyl-5,6,7,8- tetrahydroquinazolin-4-yl)- 2,3,4,5-tetrahydro-1,4- benzoxazepin-7-yl]pyridine-3- sulfonamide |
| 422 | 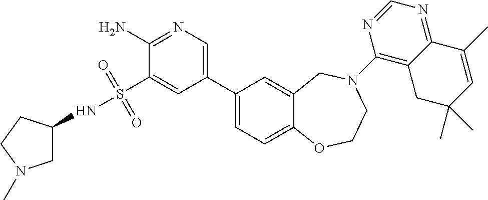 [SMILES: CC1=CC(C)(C)Cc2c1ncnc2N1CCOc2ccc(-c3cnc(N)c(S(=O)(=O)N[C@@H]4CCN(C)C4)c3)cc2C1] | 2-amino-N-[(3R)-1- methylpyrrolidin-3-yl]-5-[4- (6,6,8-trimethyl-5,6- dihydroquinazolin-4-yl)-2,3,4,5- tetrahydro-1,4-benzoxazepin-7- yl]pyridine-3-sulfonamide |
| 423 | 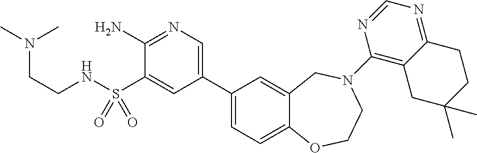 | 2-amino-N-[2- (dimethylamino)ethyl]-5-[4-(6,6- dimethyl-5,6,7,8- tetrahydroquinazolin-4-yl)- 2,3,4,5-tetrahydro-1,4- benzoxazepin-7-yl]pyridine-3- sulfonamide |
| 424 | 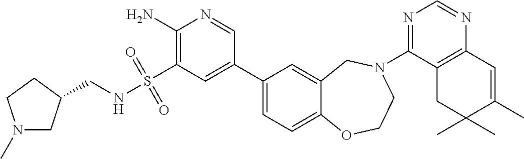 | 2-amino-N-{[(3S)-1- methylpyrrolidin-3-yl]methyl}- 5-[4-(6,6,7-trimethyl-5,6- dihydroquinazolin-4-yl)-2,3,4,5- tetrahydro-1,4-benzoxazepin-7- yl]pyridine-3-sulfonamide |
| 425 | 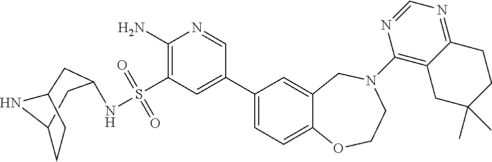 [SMILES: CC1(C)CCc2ncnc(N3CCOc4ccc(-c5cnc(N)c(S(=O)(=O)NC6CC7CCC(C6)N7)c5)cc4C3)c2C1] | 2-amino-N-8- azabicyclo[3.2.1]oct-3-yl-5-[4- (6,6-dimethyl-5,6,7,8- tetrahydroquinazolin-4-yl)- 2,3,4,5-tetrahydro-1,4- benzoxazepin-7-yl]pyridine-3- sulfanamide |
| 426 | 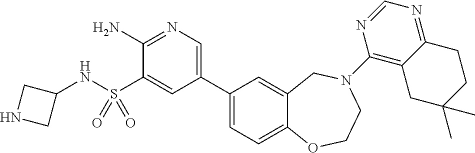 [SMILES: CC1(C)CCc2ncnc(N3CCOc4ccc(-c5cnc(N)c(S(=O)(=O)NC6CNC6)c5)cc4C3)c2C1] | 2-amino-N-azetidin-3-yl-5-[4- (6,6-dimethyl-5,6,7,8- tetrahydroquinazolin-4-yl)- 2,3,4,5-tetrahydro-1,4- benzoxazepin-7-yl]pyridine-3- sulfanamide |
| 427 | 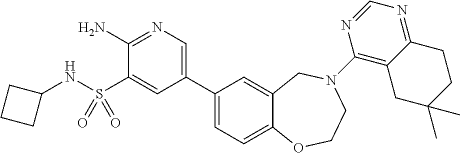 [SMILES: CC1(C)CCc2ncnc(N3CCOc4ccc(-c5cnc(N)c(S(=O)(=O)NC6CCC6)c5)cc4C3)c2C1] | 2-amino-N-cyclobutyl-5-[4-(6,6- dimethyl-5,6,7,8- tetrahydroquinazolin-4-yl)- 2,3,4,5-tetrahydro-1,4- benzoxazepin-7-yl]pyridine-3- sulfonamide |
| 428 | 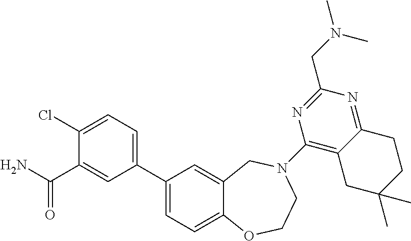 [SMILES: CN(C)Cc1nc2c(c(N3CCOc4ccc(-c5ccc(Cl)c(C(N)=O)c5)cc4C3)n1)CC(C)(C)CC2] | 2-chloro-5-(4-{2- [(dimethylamino)methyl]-6,6- dimethyl-5,6,7,8- tetrahydroquinazolin-4-yl}- 2,3,4,5-tetrahydro-1,4- benzoxazepin-7-yl)benzamide |
| 429 | 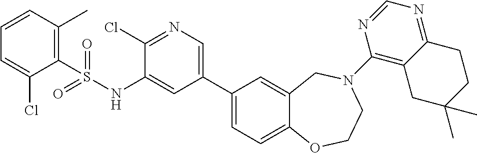 [SMILES: Cc1cccc(Cl)c1S(=O)(=O)Nc1cc(-c2ccc3c(c2)CN(c2ncnc4c2CC(C)(C)CC4)CCO3)cnc1Cl] | 2-chloro-N{2-chloro-5-[4-(6,6- dimethyl-5,6,7,8- tetrahydroquinazolin-4-yl)- 2,3,4,5-tetrahydro-1,4- benzoxazepin-7-yl]pyridin-3- yl}-6- methylbenzenesulfonamide |
| 430 | 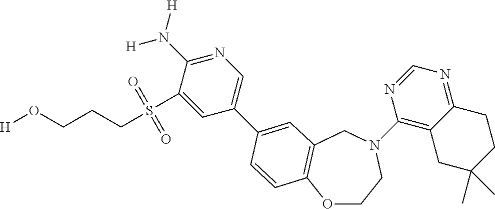 [SMILES: CC1(C)CCc2ncnc(N3CCOc4ccc(-c5cnc(N)c(S(=O)(=O)CCCO)c5)cc4C3)c2C1] | 3-({2-amino-5-[4-(6,6-dimethyl- 5,6,7,8-tetrahydroquinazolin-4- yl)-2,3,4,5-tetrahydro-1,4- benzoxazepin-7-yl]pyridin-3- yl}sulfonyl)propan-1-ol |
| 431 | 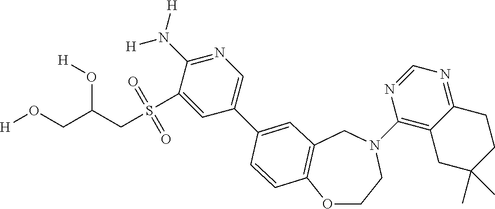 [SMILES: CC1(C)CCc2ncnc(N3CCOc4ccc(-c5cnc(N)c(S(=O)(=O)CC(O)CO)c5)cc4C3)c2C1] | 3-({2-amino-5-[4-(6,6-dimethyl- 5,6,7,8-tetrahydroquinazolin-4- yl)-2,3,4,5-tetrahydra-1,4- benzoxazepin-7-yl]pyridin-3- yl}sulfanyl)propane-1,2-diol |
| 432 | 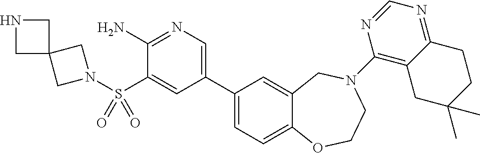 [SMILES: CC1(C)CCc2ncnc(N3CCOc4ccc(-c5cnc(N)c(S(=O)(=O)N6CC7(CNC7)C6)c5)cc4C3)c2C1] | 3-{2,6-diazaspiro[3.3]hept-2- ylsulfonyl)-5-[4-(6,6-dimethyl- 5,6,7,8-tetrahydroquinazolin-4- yl)-2,3,4,5-tetrahydro-1,4- benzoxazepin-7-yl]pyridin-2- amine |
| 433 | 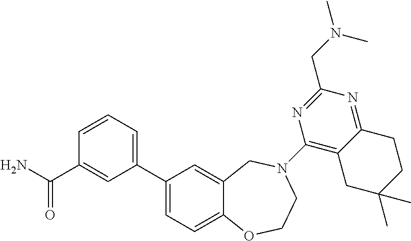 [SMILES: CN(C)Cc1nc2c(c(N3CCOc4ccc(-c5cccc(C(N)=O)c5)cc4C3)n1)CC(C)(C)CC2] | 3-(4-{2- [(dimethylamino)methyl]-6,6- dimethyl-5,6,7,8- tetrahydroquinazolin-4-yl}- 2,3,4,5-tetrahydro-1,4- benzoxazepin-7-yl)benzamide |
| 434 | 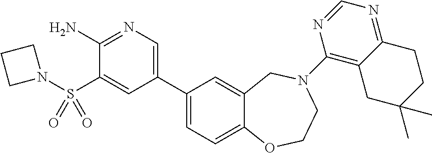 [SMILES: CC1(C)CCc2ncnc(N3CCOc4ccc(-c5cnc(N)c(S(=O)(=O)N6CCC6)c5)cc4C3)c2C1] | 3-(azetidin-1-ylsulfonyl)-5-[4- (6,6-dimethyl-5,6,7,8- tetrahydroquinazolin-4-yl)- 2,3,4,5-tetrahydro-1,4- benzoxazepin-7-yl]pyridin-2- amine |
| 435 | 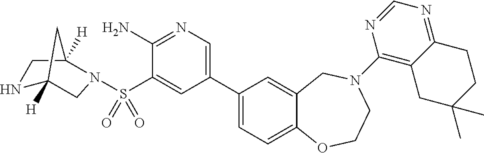 [SMILES: CC1(C)CCc2ncnc(N3CCOc4ccc(-c5cnc(N)c(S(=O)(=O)N6C[C@H]7C[C@@H]6CN7)c5)cc4C3)c2C1] | 3-[(1R,4R)-2,5- diazabicyclo[2.2.1]hept-2- ylsulfonyl]-5-[4-(6,6-dimethyl- 5,6,7,8-tetrahydroquinazolin-4- yl)-2,3,4,5-tetrahydro-1,4- benzoxazepin-7-yl]pyridin-2- amine |
| 436 | 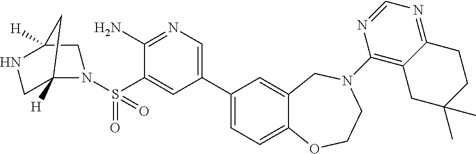 [SMILES: CC1(C)CCc2ncnc(N3CCOc4ccc(-c5cnc(N)c(S(=O)(=O)N6C[C@@H]7C[C@H]6CN7)c5)cc4C3)c2C1] | 3-[(1S,4S)-2,5- diazabicyclo[2.2.1]hept-2-ylsulfonyl]-5-[4-(6,6-dimethyl-5,6,7,8-tetrahydroquinazolin-4-yl)-2,3,4,5-tetrahydro-1,4-benzoxazepin-7-yl]pyridin-2-amine |
| 437 | 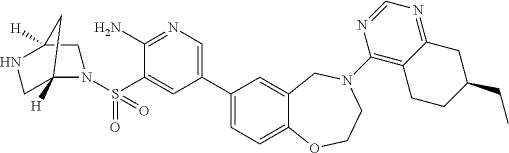 [SMILES: CC[C@H]1CCc2c(ncnc2N2CCOc3ccc(-c4cnc(N)c(S(=O)(=O)N5C[C@@H]6C[C@H]5CN6)c4)cc3C2)C1] | 3-[1S,4S)-2,5- diazabicyclo[2.2.1]hept-2- ylsulfonyl]-5-{4-[(7S)-7-ethyl- 5,6,7,8-tetrahydroquinazolin-4- yl]-2,3,4,5-tetrahydro-1,4- benzoxazepin-7-y]pyridin-2- amine |
| 438 | 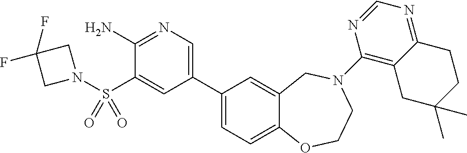 [SMILES: CC1(C)CCc2ncnc(N3CCOc4ccc(-c5cnc(N)c(S(=O)(=O)N6CC(F)(F)C6)c5)cc4C3)c2C1] | 3-[(3,3-difluoroazetidin-1- yl)sulfonyl]-5-[4-(6,6-dimethyl- 5,6,7,8-tetrahydroquinazolin-4- yl)-2,3,4,5-tetrahydra-1,4- benzoxazepin-7-yl]pyridin-2- amine |
| 439 | 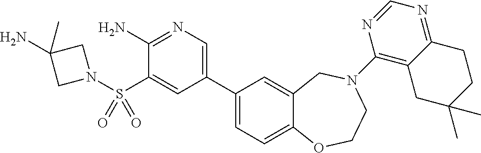 [SMILES: CC1(C)CCc2ncnc(N3CCOc4ccc(-c5cnc(N)c(S(=O)(=O)N6CC(C)(N)C6)c5)cc4C3)c2C1] | 3-[(3-amino-3-methylazetidin-1- yl)sulfonyl]-5-[4-(6,6-dimethyl- 5,6,7,8-tetrahydroquinazolin-4- yl)-2,3,4,5-tetrahydro-1,4- benzoxazepin-7-yl]pyridin-2- amine |
| 440 | 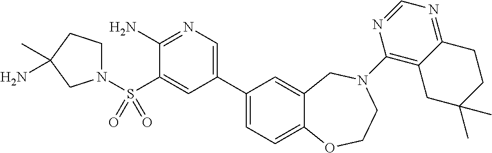 [SMILES: CC1(C)CCc2ncnc(N3CCOc4ccc(-c5cnc(N)c(S(=O)(=O)N6CCC(C)(N)C6)c5)cc4C3)c2C1] | 3-[(3-amino-3-methylpyrrolidin- l-yl)sulfonyl]-5-[4-(6,6- dimethyl-5,6,7,8- tetrahydroquinazolin-4-yl)- 2,3,4,5-tetrahydro-1,4- benzoxazepm-7-yl]pyridin-2- amine |
| 441 | 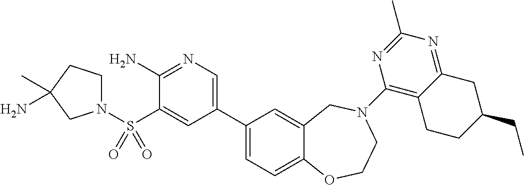 [SMILES: CC[C@H]1CCc2c(nc(C)nc2N2CCOc3ccc(-c4cnc(N)c(S(=O)(=O)N5CCC(C)(N)C5)c4)cc3C2)C1] | 3-[(3-amino-3-methylpyrrolidin- 1-yl)sulfonyl]-5-{4-[(7S)-7- ethyl-2-methyl-5,6,7,8- tetrahydroquinazolin-4-yl]- 2,3,4,5-tetrahydro-1,4- benzoxazepin-7-yl}pyridin-2- amine |
| 442 | 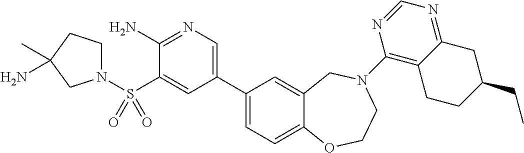 [SMILES: CC[C@H]1CCc2c(ncnc2N2CCOc3ccc(-c4cnc(N)c(S(=O)(=O)N5CCC(C)(N)C5)c4)cc3C2)C1] | 3-[(3-amino-3-methylpyrrolidin- 1-yl)sulfonyl]-5-{4-[(7S)-7- ethyl-5,6,7,8- tetrahydroquinazolin-4-yl]- 2,3,4,5-tetrahydro-1,4- benzoxazepin-7-yl}pyridin-2- amine |
| 443 | 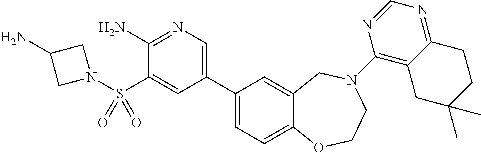 [SMILES: CC1(C)CCc2ncnc(N3CCOc4ccc(-c5cnc(N)c(S(=O)(=O)N6CC(N)C6)c5)cc4C3)c2C1] | 3-[(3-aminoazetidin-1- yl)sulfonyl]-5-[4-(6,6-dimethyl- 5,6,7,8-tetrahydroquinazolin-4- yl)-2,3,4,5-tetrahydro-1,4- benzoxazepin-7-yl]pyridin-2- amine |
| 444 | 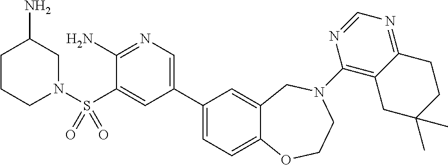 [SMILES: CC1(C)CCc2ncnc(N3CCOc4ccc(-c5cnc(N)c(S(=O)(=O)N6CCCC(N)C6)c5)cc4C3)c2C1] | 3-[{3-aminopiperidin-1- yl)sulfonyl]-5-[4-(6,6-dimethyl- 5,6,7,8-tetrahydroquinazolin-4- yl)-2,3,4,5-tetrahydro-1,4- benzoxazepin-7-yl]pyridin-2- amine |
| 445 | 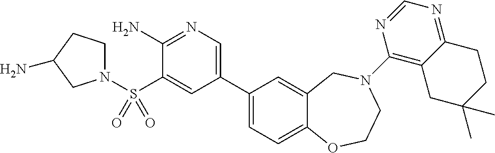 [SMILES: CC1(C)CCc2ncnc(N3CCOc4ccc(-c5cnc(N)c(S(=O)(=O)N6CCC(N)C6)c5)cc4C3)c2C1] | 3-[(3-aminopyrrolidin-1- yl)sulfonyl]-5-[4-(6,6-dimethyl- 5,6,7,8-tetrahydroquinazolin-4- yl)-2,3,4,5-tetrahydro-1,4- benzoxazepin-7-yl]pyridin-2- amine |
| 446 | 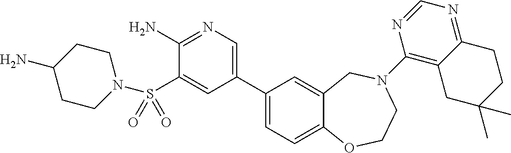 [SMILES: CC1(C)CCc2ncnc(N3CCOc4ccc(-c5cnc(N)c(S(=O)(=O)N6CCC(N)CC6)c5)cc4C3)c2C1] | 3-[(4-aminopiperidin-1- yl)sulfonyl]-5-[4-(6,6-dimethyl- 5,6,7,8-tetrahydroquinazolin-4- yl)-2,3,4,5-tetrahydro-1,4- benzoxazepin-7-yl]pyridin-2- amine |
| 447 | 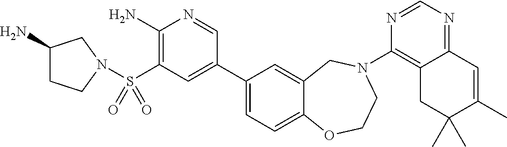 [SMILES: CC1=Cc2ncnc(N3CCOc4ccc(-c5cnc(N)c(S(=O)(=O)N6CC[C@@H](N)C6)c5)cc4C3)c2CC1(C)C] | 3-{[(3R)-3-aminopyrrolidin-1-yl]sulfonyl}-5-[4-(6,6,7-trimethyl-5,6-dihydroquinazolin-4-yl)-2,3,4,5-tetrahydro-1,4-benzoxazepin-7-yl]pyridin-2- amine |
| 448 | 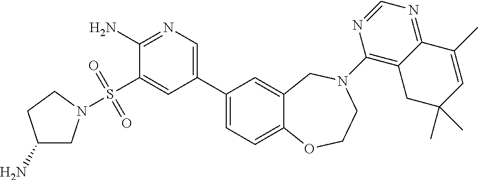 [SMILES: CC1=CC(C)(C)Cc2c1ncnc2N1CCOc2ccc(-c3cnc(N)c(S(=O)(=O)N4CC[C@@H](N)C4)c3)cc2C1] | 3-{[(3R)-3-aminopyrrolidin-1- yl]sulfonyl}-5-[4-(6,6,8- trimethyl-5,6-dihydroquinazolin- 4-y])-2,3,4,5-tetrahydro-1,4- benzoxazepin-7-yl]pyridin-2- amine |
| 449 | 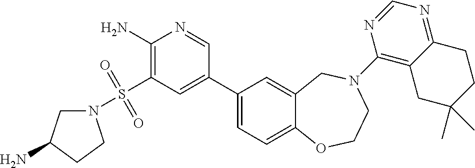 [SMILES: CC1(C)CCc2ncnc(N3CCOc4ccc(-c5cnc(N)c(S(=O)(=O)N6CC[C@@H](N)C6)c5)cc4C3)c2C1] | 3-{[(3R)-3-aminopyrrolidin-1-yl]sulfonyl}5-[4-(6,6-dimethyl- 5,6,7,8-tetrahydroquinazolin-4-yl)-2,3,4,5-tetrahydro-1,4-benzoxazepin-7-yl]pyridin-2-amine |
| 450 | 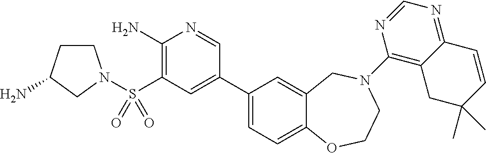 [SMILES: CC1(C)C=Cc2ncnc(N3CCOc4ccc(-c5cnc(N)c(S(=O)(=O)N6CC[C@@H](N)C6)c5)cc4C3)c2C1] | 3-{[(3R)-3-aminopyrrolidin-1- yl]sulfonyl}-5-[4-(6,6-dimethyl- 5,6-dihydroquinazolin-4-yl)- 2,3,4,5-tetrahydro-1,4- benzoxazepin-7-yl]pyridin-2- amine |
| 451 | 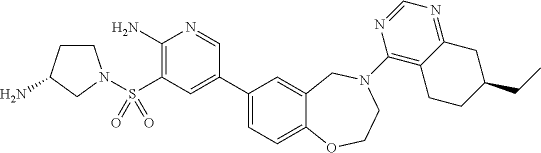 [SMILES: CC[C@H]1CCc2c(ncnc2N2CCOc3ccc(-c4cnc(N)c(S(=O)(=O)N5CC[C@@H](N)C5)c4)cc3C2)C1] | 3-{[(3R)-3-aminopyrrolidin-1- yl]sulfonyl}-5-{4-[(7S)-7-ethyl- 5,6,7,8-tetrahydroquinazolin-4- yl]-2,3,4,5-tetrahydro-1,4- benzoxazepin-7-yl}pyridin-2- amine |
| 452 | 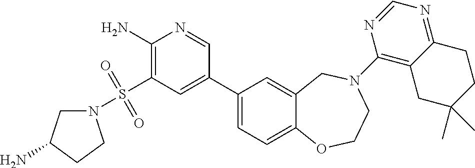 [SMILES: CC1(C)CCc2ncnc(N3CCOc4ccc(-c5cnc(N)c(S(=O)(=O)N6CC[C@H](N)C6)c5)cc4C3)c2C1] | 3-{[(3S)-3-aminopyrrolidin-1- yl]sulfonyl}-5-[4-(6,6-dimethyl- 5,6,7,8-tetrahydroquinazolin-4- yl)-2,3,4,5-tetrahydro-1,4- benzoxazepin-7-yl]pyridin-2- amine |
| 453 | 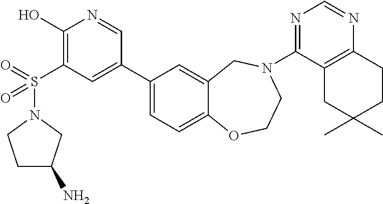 [SMILES: CC1(C)CCc2ncnc(N3CCOc4ccc(-c5cnc(O)c(S(=O)(=O)N6CC[C@H](N)C6)c5)cc4C3)c2C1] | 3-{[(3S)-3-aminopyrrolidin-1- yl]sulfonyl}-5-[4-(6,6-dimethyl- 5,6,7,8-tetrahydroquinazolin-4- yl)-2,3,4,5-tetrahydro-1,4- benzoxazepin-7-yl]pyridin-2-ol |
| 454 | 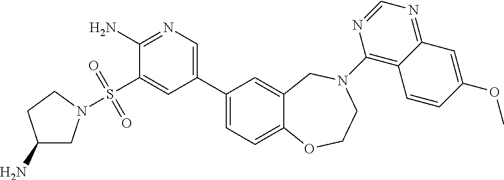 [SMILES: COc1ccc2c(N3CCOc4ccc(-c5cnc(N)c(S(=O)(=O)N6CC[C@H](N)C6)c5)cc4C3)ncnc2c1] | 3-{[(3S)-3-aminopyrrolidin-1- yl]sulfonyl}-5-{4-[7- (methyloxy)quinazolin-4-yl]- 2,3,4,5-tetrahydro-1,4- benzoxazepin-7-yl}pyridin-2- amine |
| 455 | 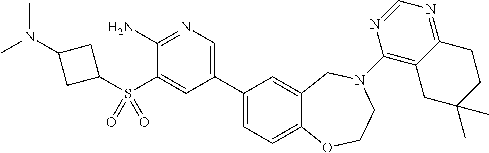 | 3-{[3-(dimethylamino)azetidin- 1-yl]sulfonyl)-5-[4-(6,6- dimethyl-5,6,7,8- tetrahydroquinazolin-4-yl)- 2,3,4,5-tetrahydro-1,4- benzoxazepin-7-yl]pyridin-2- amine |
| 456 | 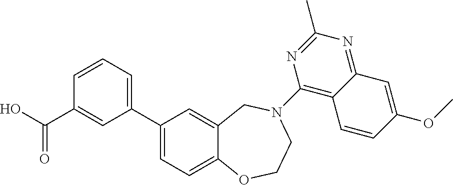 [SMILES: COc1ccc2c(N3CCOc4ccc(-c5cccc(C(=O)O)c5)cc4C3)nc(C)nc2c1] | 3-{4-[2-methyl-7- (methyloxy)quinazolin-4-yl]- 2,3,4,5-tetrahydro-1,4- benzoxazepin-7-yl}benzoic acid |
| 457 | 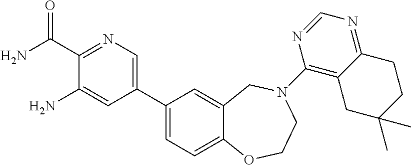 [SMILES: CC1(C)CCc2ncnc(N3CCOc4ccc(-c5cnc(C(N)=O)c(N)c5)cc4C3)c2C1] | 3-amino-5-[4-(6,6-dimethyl- 5,6,7,8-tetrahydroquinazolin-4- yl)-2,3,4,5-tetrahydro-1,4- benzoxazepin-7-yl]pyridine-2- carboxamide |
| 458 | 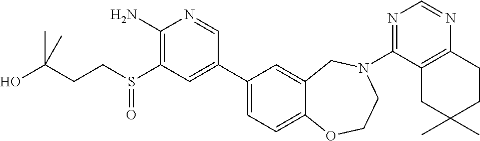 [SMILES: CC(C)(O)CCS(=O)c1cc(-c2ccc3c(c2)CN(c2ncnc4c2CC(C)(C)CC4)CCO3)cnc1N] | 4-({2-amino-5- [4-(6,6-dimethyl- 5,6,7,8-tetrahydroquinazolin-4- yl)-2,3,4,5-tetrahydro-1,4- benzoxazepin-7-yl]pyridin-3- yl}sulfinyl)-2-methylbutan-2-ol |
| 459 | 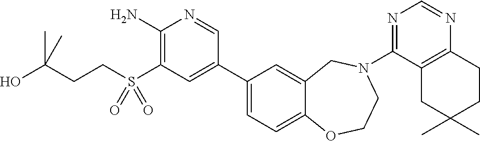 [SMILES: CC(C)(O)CCS(=O)(=O)c1cc(-c2ccc3c(c2)CN(c2ncnc4c2CC(C)(C)CC4)CCO3)cnc1N] | 4-({2-amino-5-[4-(6,6-dimethyl- 5,6,7,8-tetrahydroquinazolin-4- yl)-2,3,4,5-tetrahydro-1,4- benzoxazepin-7-yl]pyridin-3- yl}sulfonyl)-2-methylbutan-2-ol |
| 460 | 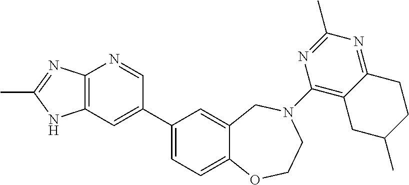 [SMILES: Cc1nc2c(c(N3CCOc4ccc(-c5cnc6nc(C)[nH]c6c5)cc4C3)n1)CC(C)CC2] | 4-(2,6-dimethyl-5,6,7,8- tetrahydroquinazolin-4-yl)-7-(2- methyl-1H-imidazo[4,5- b]pyridin-6-yl)-2,3,4,5- tetrahydro-1,4-benzoxazepine |
| 461 | 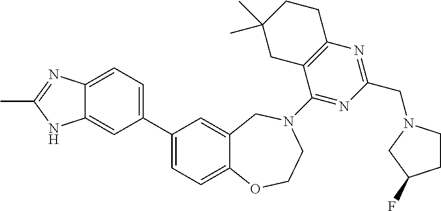 [SMILES: Cc1nc2ccc(-c3ccc4c(c3)CN(c3nc(CN5CC[C@@H](F)C5)nc5c3CC(C)(C)CC5)CCO4)cc2[nH]1] | 4-(2-{[(3R)-3-fluoropyrrolidin- 1-yl]methyl}-6,6-dimethyl- 5,6,7,8-tetrahydroquinazolin-4- yl)-7-(2-methyl-1H- benzimidazol-6-yl)-2,3,4,5- tetrahydro-1,4-benzoxazepine |
| 462 | 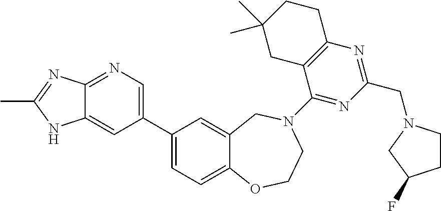 [SMILES: Cc1nc2ncc(-c3ccc4c(c3)CN(c3nc(CN5CC[C@@H](F)C5)nc5c3CC(C)(C)CC5)CCO4)cc2[nH]1] | 4-(2-{[(3R)-3-fluoropyrrolidin-1-yl]methyl}-6,6-dimethyl- 5,6,7,8-tetrahydroquinazolin-4- yl)-7-(2-methyl-1H-imidazo[4,5- b]pyridin-6-yl)-2,3,4,5- tetrahydro-1,4-benzoxazepine |
| 463 | 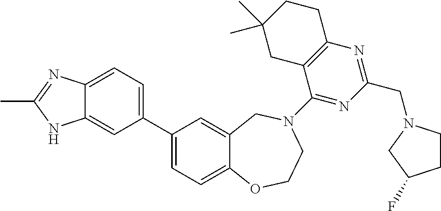 [SMILES: Cc1nc2ccc(-c3ccc4c(c3)CN(c3nc(CN5CC[C@H](F)C5)nc5c3CC(C)(C)CC5)CCO4)cc2[nH]1] | 4-(2-{[(3S)-3-fluoropyrrolidin-1- yl]methyl}-6,6-dimethyl-5,6,7,8- tetrahydroquinazolin-4-yl)-7-(2- methyl-1H-benzimidazol-6-yl)- 2,3,4,5-tetrahydro-1,4- benzoxazepine |
| 464 | 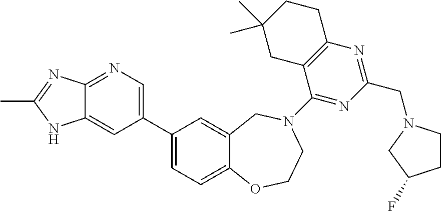 [SMILES: Cc1nc2ncc(-c3ccc4c(c3)CN(c3nc(CN5CC[C@H](F)C5)nc5c3CC(C)(C)CC5)CCO4)cc2[nH]1] | 4-(2-{[(3S)-3-fluoropyrrolidin-1- yl}methyl}-6,6-dimethyl-5,6,7,8- tetrahydroquinazolin-4-yl)-7-(2- methyl-1H-imidazo[4,5- b]pyridin-6-yl)-2,3,4,5- tetrahydro-1,4-benzoxazepine |
| 465 | 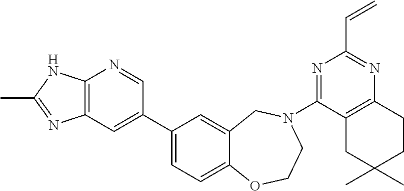 | 4-(2-ethenyl-6,6-dimethyl- 5,6,7,8-tetrahydroquinazolin-4- yl)-7-(2-methyl-3H-imidazo[4,5- b]pyridin-6-yl)-2,3,4,5- tetrahydro-1,4-benzoxazepine |
| 466 | 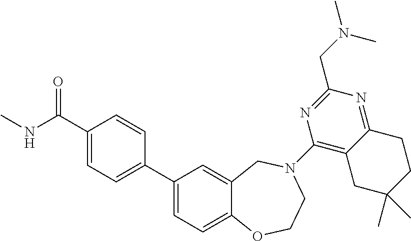 | 4-(4-{2- [(dimethylamino)methyl]-6,6- dimethyl-5,6,7,8- tetrahydroquinazolin-4-yl}- 2,3,4,5-tetrahydro-1,4- benzoxazepin-7-yl)-N methylbenzamide |
| 468 | 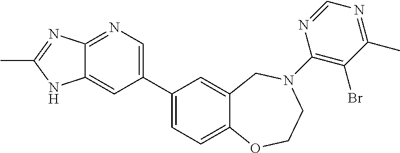 | 4-(5-bromo-6-methylpyrimidin- 4-yl)-7-(2-methyl-1H- imidazo[4,5-b]pyridin-6-yl)- 2,3,4,5-tetrahydro-1,4- benzoxazepine |
| 469 | 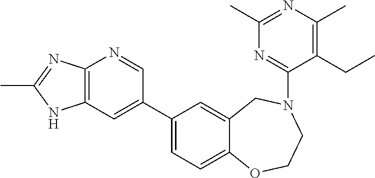 [SMILES: CCc1c(C)nc(C)nc1N1CCOc2ccc(-c3cnc4nc(C)[nH]c4c3)cc2C1] | 4-(5-ethyl-2,6- dimethylpyrimidin-4-yl)-7-(2- methyl-1H-imidazo[4,5- b]pyridin-6-yl)-2,3,4,5- tetrahydro-1,4-benzoxazepine |
| 470 | 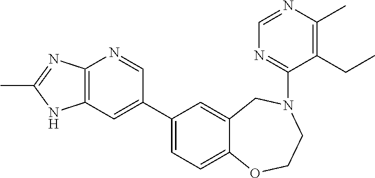 [SMILES: CCc1c(C)ncnc1N1CCOc2ccc(-c3cnc4nc(C)[nH]c4c3)cc2C1] | 4-(5-ethyl-6-methylpyrimidin-4- yl)-7-(2-methyl-1H-imidazo[4,5- b]pyridin-6-yl)-2,3,4,5- tetrahydro-1,4-benzoxazepine |
| 471 | 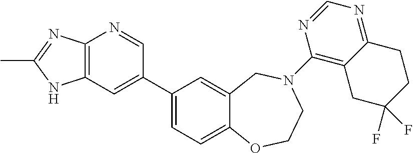 [SMILES: Cc1nc2ncc(-c3ccc4c(c3)CN(c3ncnc5c3CC(F)(F)CC5)CCO4)cc2[nH]1] | 4-(6,6-difluoro-5,6,7,8- tetrahydroquinazolin-4-yl)-7-(2- methyl-1H-imidazo[4,5- b]pyridin-6-yl)-2,3,4,5- tetrahydro-1,4-benzoxazepine |
| 472 | 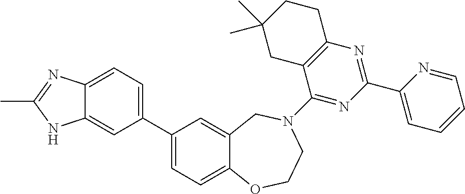 [SMILES: Cc1nc2ccc(-c3ccc4c(c3)CN(c3nc(-c5ccccn5)nc5c3CC(C)(C)CC5)CCO4)cc2[nH]1] | 4-(6,6-dimethyl-2-pyridin-2-yl- 5,6,7,8-tetrahydroquinazolin-4- yl)-7-(2-methyl-1H- benzimidazol-6-yl)-2,3,4,5- tetrahydro-1,4-benzoxazepine |
| 473 | 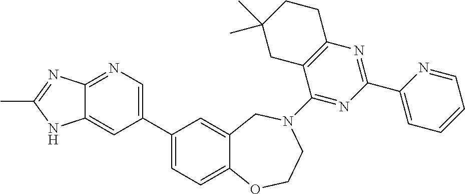 [SMILES: Cc1nc2ncc(-c3ccc4c(c3)CN(c3nc(-c5ccccn5)nc5c3CC(C)(C)CC5)CCO4)cc2[nH]1] | 4-(6,6-dimethyl-2-pyridin-2-yl- 5,6,7,8-tetrahydroquinazolin-4- yl)-7-(2-methyl-1H-imidazo[4,5- b]pyridin-6-yl)-2,3,4,5- tetrahydro-1,4-benzoxazepine |
| 474 | 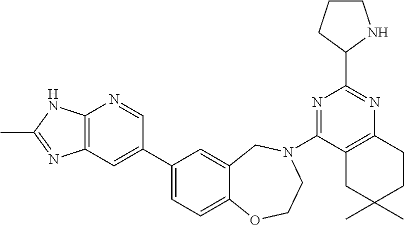 | 4-(6,6-dimethyl-2-pyrrolidin-2- yl-5,6,7,8-tetrahydroquinazolin- 4-yl)-7-(2-methyl-3H- imidazo[4,5-b]pyridin-6-yl)- 2,3,4,5-tetrahydro-1,4- benzoxazepine |
| 475 | 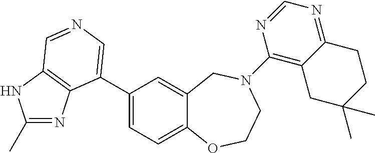 | 4-(6,6-dimethyl-5,6,7,8- tetrahydroquinazolin-4-yl)-7-(2- methyl-3H-imidazo[4,5- c]pyridin-7-yl)-2,3,4,5- tetrahydro-1,4-benzoxazepine |
| 476 | 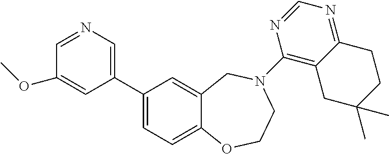 | 4-(6,6-dimethyi-5,6,7,8- tetrahydroquinazolin-4-yl)-7-[5- (methyloxy)pyridin-3-yl]- 2,3,4,5-tetrahydro-1,4- benzoxazepine |
| 477 | 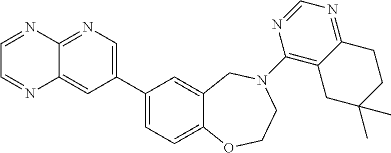 [SMILES: CC1(C)CCc2ncnc(N3CCOc4ccc(-c5cnc6nccnc6c5)cc4C3)c2C1] | 4-(6,6-dimethyl-5,6,7,8- tetrahydroquinazolin-4-yl)-7- pyrido[2,3-b]pyrazin-7-yl- 2,3,4,5-tetrahydro-1,4- benzoxazepine |
| 478 | 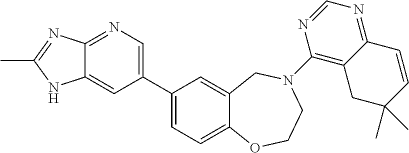 | 4-(6,6-dimethyl-5,6- dihydroquinazolin-4-yl)-7-(2- methyl-1H-imidazo[4,5- b]pyridin-6-yl)-2,3,4,5- tetrahydro-1,4-benzoxazepine |
| 479 | 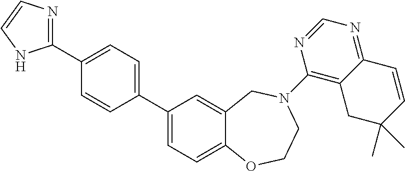 [SMILES: CC1(C)C=Cc2ncnc(N3CCOc4ccc(-c5ccc(-c6ncc[nH]6)cc5)cc4C3)c2C1] | 4-(6,6-dimethyl-5,6- dihydroquinazolin-4-yl)-7-[4- (1H-imidazol-2-yl)phenyl]- 2,3,4,5-tetrahydro-1,4- benzoxazepine |
| 480 | 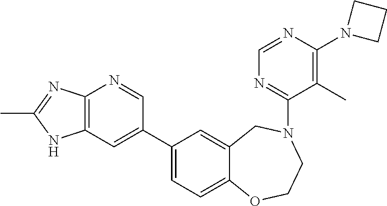 [SMILES: Cc1nc2ncc(-c3ccc4c(c3)CN(c3ncnc(N5CCC5)c3C)CCO4)cc2[nH]1] | 4-(6-azetidin-1-yl-5- methylpyrimidin-4-yl)-7-(2- methyl-1H-imidazo[4,5- b]pyridin-6-yl)-2,3,4,5- tetrahydro-1,4-benzoxazepine |
| 481 | 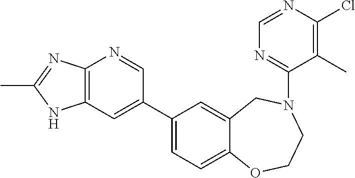 [SMILES: Cc1nc2ncc(-c3ccc4c(c3)CN(c3ncnc(Cl)c3C)CCO4)cc2[nH]1] | 4-(6-chloro-5-methylpyrimidin- 4-yl)-7-(2-methyl-1H- imidazo[4,5-b]pyridin-6-yl)- 2,3,4,5-tetrahydro-1,4- benzoxazepine |
| 482 | 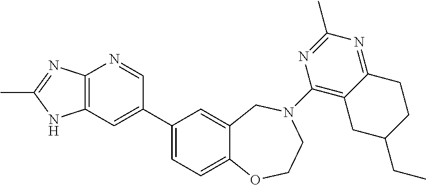 [SMILES: CCC1CCc2nc(C)nc(N3CCOc4ccc(-c5cnc6nc(C)[nH]c6c5)cc4C3)c2C1] | 4-(6-ethyl-2-methyl-5,6,7,8- tetrahydroquinazolin-4-yl)-7-(2- methyl-1H-imidazo[4,5- b]pyridin-6-yl)-2,3,4,5- tetrahydro-1,4-benzoxazepine |
| 483 | 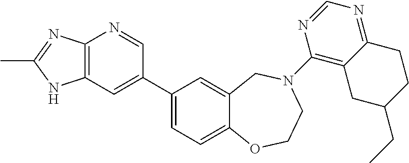 [SMILES: CCC1CCc2ncnc(N3CCOc4ccc(-c5cnc6nc(C)[nH]c6c5)cc4C3)c2C1] | 4-(6-ethyl-2-methyl-5,6,7,8- tetrahydroquinazolin-4-yl)-7-(2- methyl-1H-imidazo[4,5- b]pyridin-6-yl)-2,3,4,5- tetrahydro-1,4-benzoxazepine |
| 484 | 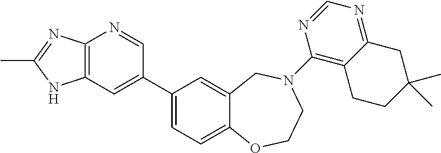 | 4-(7,7-dimethyl-5,6,7,8- tetrahydroquinazolin-4-yl)-7-(2- methyl-1H-imidazo[4,5- b]pyridin- 6-yl)-2,3,4,5- tetrahydro-1,4-benzoxazepine |
| 485 | 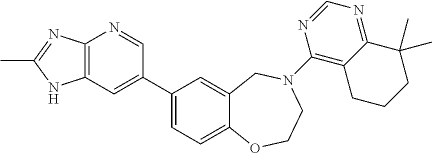 | 4-(8,8-dimethyl-5,6,7,8- tetrahydroquinazolin-4-yl)-7-(2- methyl-1H-imidazo[4,5- b]pyridin-6-yl)-2,3,4,5- tetrahydro-1,4-benzoxazepine |
| 486 | 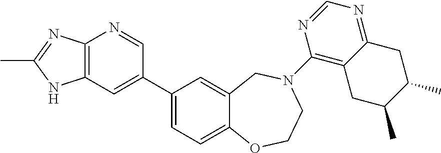 | 4-[(6S,7S)-6,7-dimethyl-5,6,7,8- tetrahydroquinazolin-4-yl]-7-(2- methyl-1H-imidazo[4,5- b]pyridin-6-yl)-2,3,4,5- tetrahydro-1,4-benzoxazepine |
| 487 | 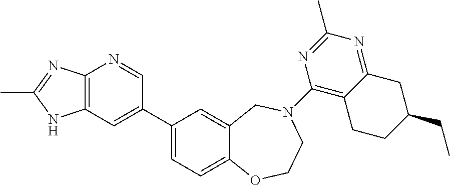 [SMILES: CC[C@H]1CCc2c(nc(C)nc2N2CCOc3ccc(-c4cnc5nc(C)[nH]c5c4)cc3C2)C1] | 4-[(7S)-7-ethyl-2-methyl- 5,6,7,8-tetrahydroquinazolin-4- yl]-7-(2-methyl-1H-imidazo[4,5- b]pyridin-6-yl)-2,3,4,5- tetrahydro-1,4-benzoxazepine |
| 488 | 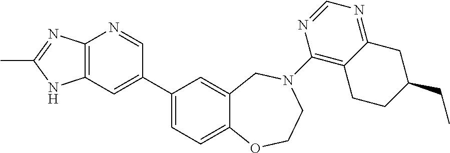 [SMILES: CC[C@H]1CCc2c(ncnc2N2CCOc3ccc(-c4cnc5nc(C)[nH]c5c4)cc3C2)C1] | 4-[(7S)-7-ethyl-5,6,7,8- tetrahydroquinazolin-4-yl]-7-(2- methyl-1H-imidazo[4,5- b]pyridin-6-yl)-2,3,4,5- tetrahydro-1,4-benzoxazepine |
| 489 | 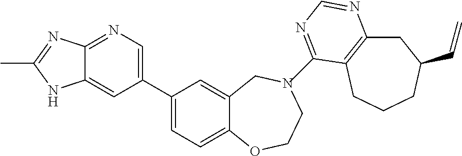 | 4-[(8S)-8-ethenyl-6,7,8,9- tetrahydro-5H- cyclohepta[d]pyrimidin-4-yl]-7- (2-methyl-1H-imidazo[4,5- b]pyridin-6-yl)-2,3,4,5- tetrahydro-1,4-benzoxazepine |
| 490 | 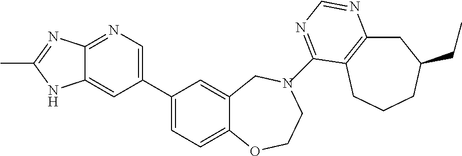 | 4-[(8S)-8-ethyl-6,7,8,9- tetrahydro-5H- cyclohepta[d]pyrimidin-4-yl]-7- (2-methyl-1H-imidazo[4,5- b]pyridin-6-yl)-2,3,4,5- tetrahydro-1,4-benzoxazepine |
| 491 | 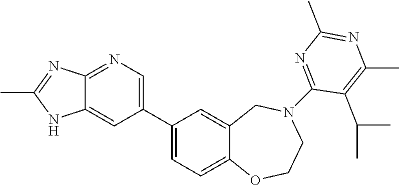 | 4-[2,6-dimethyl-5-(1- methylethyl)pyrimidin-4-yl]-7- (2-methyl-1H-imidazo[4,5- b)pyridin-6-yl)-2,3,4,5- tetrahydro-1,4-benzoxazepine |
| 492 | 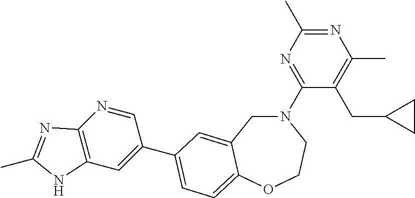 [SMILES: Cc1nc(C)c(CC2CC2)c(N2CCOc3ccc(-c4cnc5nc(C)[nH]c5c4)cc3C2)n1] | 4-[5-(cyclopropylmethyl)-2,6- dimethylpyrimidin-4-yl]-7-(2- methyl-1H-imidazo[4,5- b]pyridin-6-yl)-2,3,4,5- tetrahydro-1,4-benzoxazepine |
| 493 | 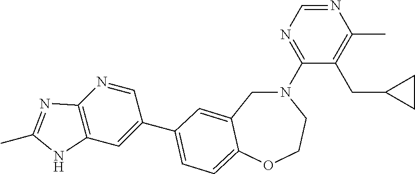 [SMILES: Cc1nc2ncc(-c3ccc4c(c3)CN(c3ncnc(C)c3CC3CC3)CCO4)cc2[nH]1] | 4-[5-(cyclopropylmethyl)-6- methylpyrimidin-4-yl]-7-(2- methyl-1H-imidazo[4,5- b]pyridin-6-y])-2,3,4,5- tetrahydro-1,4-benzoxazepine |
| 494 | 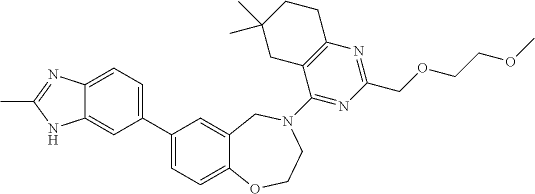 | 4-[6,6-dimethyl-2-({[2- (methyloxy)ethyl]oxy}methyl)- 5,6,7,8-tetrahydroquinazolin-4- yl]-7-(2-methyl-1H- benzimidazol-6-yl)-2,3,4,5- tetrahydro- 1,4-benzoxazepine |
| 495 | 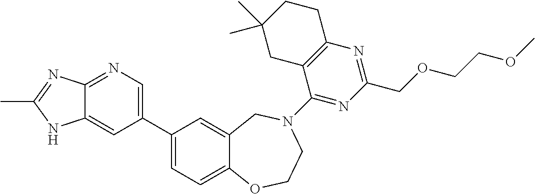 | 4-[6,6-dimethyl-2-({[2- (methyloxy)ethyl]oxy}methyl)- 5,6,7,8-tetrahydroquinazolin-4- yl]-7-(2-methyl-1H-imidazo[4,5- b]pyridin-6-yl)-2,3,4,5- tetrahydro-1,4-benzoxazepine |
| 496 | 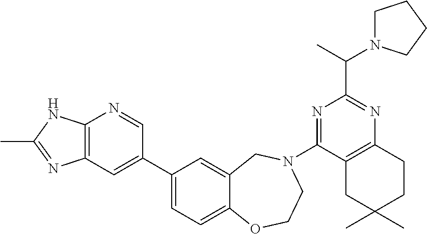 [SMILES: Cc1nc2cc(-c3ccc4c(c3)CN(c3nc(C(C)N5CCCC5)nc5c3CC(C)(C)CC5)CCO4)cnc2[nH]1] | 4-[6,6-dimethyl-2-(1-pyrrolidin- 1-ylethyl)-5,6,7,8- tetrahydroquinazolin-4-yl]-7-(2- methyl-3H-imidazo[4,5- b]pyridin-6-yl)-2,3,4,5- tetrahydro-1,4-benzoxazepine |
| 497 | 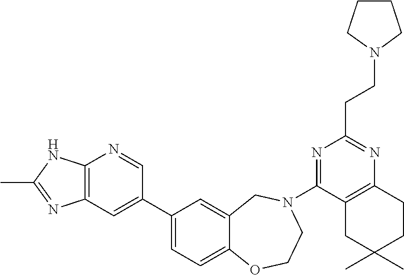 | 4-[6,6-dimethyl-2-(2-pyrrolidin- 1-ylethyl)-5,6,7,8- tetrahydroquinazolin-4-yl]-7-(2- methyl-3H-imidazo[4,5- b]pyridin-6-yl)-2,3,4,5- tetrahydro-1,4-benzoxazepine |
| 498 | 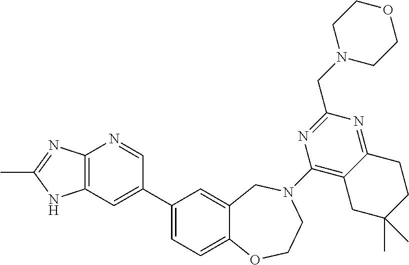 | 4-[6,6-dimethyl-2-(morpholin-4- ylethyl)-5,6,7,8- tetrahydroquinazolin-4-yl]-7-(2- methyl-1H-imidazo[4,5- b]pyridin-6-yl)-2,3,4,5- tetrahydro-1,4-benzoxazepine |
| 499 | 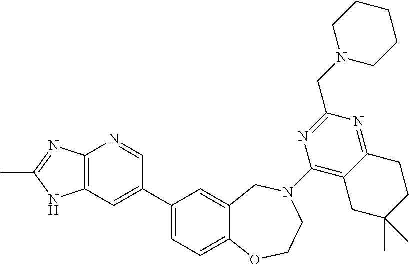 | 4-[6,6-dimethyl-2-(piperidin-1- ylmethyl)-5,6,7,8- tetrahydroquinazolin-4-yl]-7-(2- methyl-1H-imidazo[4,5- b]pyridin-6-yl)-2,3,4,5- tetrahydro-1,4-benzoxazepine |
| 500 | 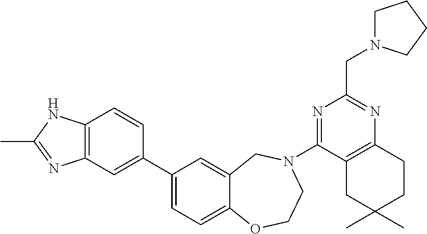 [SMILES: Cc1nc2cc(-c3ccc4c(c3)CN(c3nc(CN5CCCC5)nc5c3CC(C)(C)CC5)CCO4)ccc2[nH]1] | 4-[6,6-dimethyl-2-(pyrrolidin-1- ylmethyl)-5,6,7,8- tetrahydroquinazolin-4-yl]-7-(2- methyl-1H-benzimidazol-5-yl)- 2,3,4,5-tetrahydro-1,4- benzoxazepine |
| 501 | 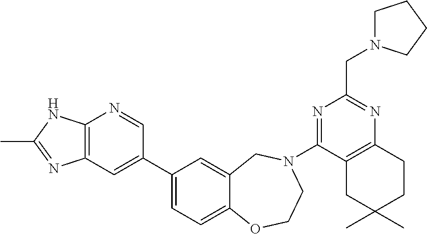 [SMILES: Cc1nc2cc(-c3ccc4c(c3)CN(c3nc(CN5CCCC5)nc5c3CC(C)(C)CC5)CCO4)cnc2[nH]1] | 4-[6,6-dimethyl-2-(pyrrolidin-1- ylmethyl)-5,6,7,8- tetrahydroquinazolin-4-yl]-7-(2- methyl-3H-imidazo[4,5- b]pyridin-6-yl)-2,3,4,5- tetrahydro-1,4-benzoxazepine |
| 502 | 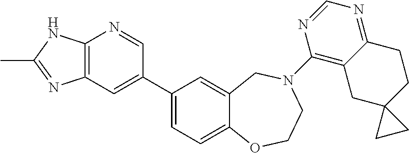 [SMILES: Cc1nc2cc(-c3ccc4c(c3)CN(c3ncnc5c3CC3(CC5)CC3)CCO4)cnc2[nH]1] | 4′-[7-(2-methyl-3H-imidazo[4,5- b]pyridin-6-yl)-2,3-dihydro-1,4- benzoxazepin-4(5H)-yl]-7′,8′- dihydro-5′H-spiro[cyclopropane- 1,6′-quinazoline] |
| 503 | 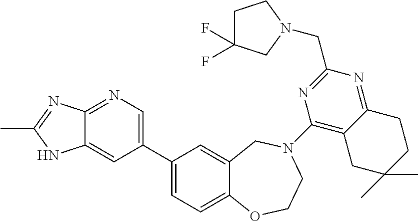 | 4-{2-[(3,3-difluoropyrrolidin-1- yl)methyl]-6,6-dimethyl-5,6,7,8- tetrahydroquinazolin-4-yl}-7-(2- methyl-1H-imidazo[4,5- b]pyridin-6-yl-2,3,4,5- tetrahydro-1,4-benzoxazepine |
| 504 | 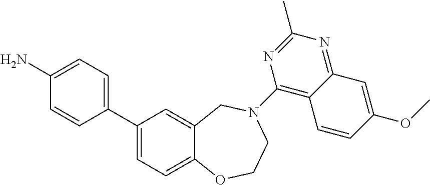 [SMILES: COc1ccc2c(N3CCOc4ccc(-c5ccc(N)cc5)cc4C3)nc(C)nc2c1] | 4-{4-[2-methyl-7- (methyloxy)quinazolin-4-yl]- 2,3,4,5-tetrahydro-1,4- benzoxazepin-7-yl}aniline |
| 505 | 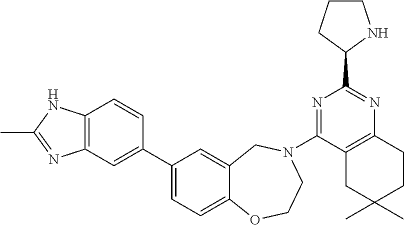 | 4-{6,6-dimethyl-2-[(2R)- pyrrolidin-2-yl]-5,6,7,8- tetrahydroquinazolin-4-yl)-7-(2- methyl-1H-benzimidazol-5-yl)- 2,3,4,5-tetrahydro-1,4- benzoxazepine |
| 506 | 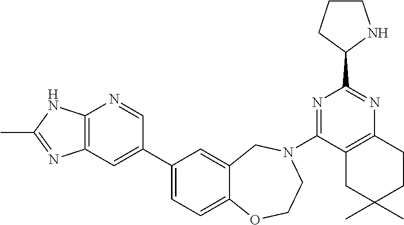 | 4-{6,6-dimethyl-2-[(2R)- pyrrolidin-2-yl]-5,6,7,8- tetrahydroquinazolin-4-yl}-7-(2- methyl-3H-imidazo[4,5- b]pyridin-6-yl)-2,3,4,5- tetrahydro-1,4-benzoxazepine |
| 507 | 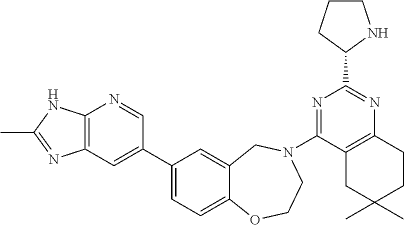 | 4-{6,6-dimethyl-2-[(2S)- pyrrolidin-2-yl]-5,6,7,8- tetrahydroquinazolin-4-yl}-7-(2- methyl-3H-imidazo[4,5- b]pyridin-6-yl)-2,3,4,5- tetrahydro-1,4-benzoxazepine |
| 508 | 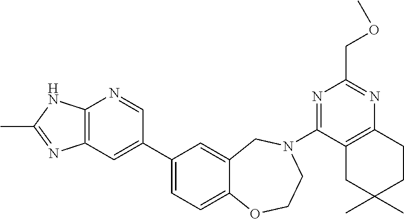 | 4-{6,6-dimethyl-2- [(methyloxy)methyl]-5,6,7,8- tetrahydroquinazolin-4-yl}-7-(2- methyl-3H-imidazo[4,5- b]pyridin-6-yl)-2,3,4,5- tetrahydro-1,4-benzoxazepine |
| 509 | 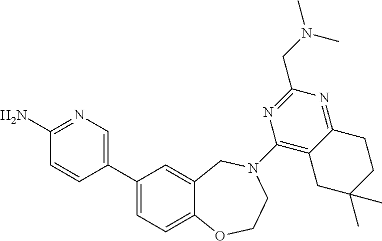 [SMILES: CN(C)Cc1nc2c(c(N3CCOc4ccc(-c5ccc(N)nc5)cc4C3)n1)CC(C)(C)CC2] | 5-(4-{2- [(dimethylamino)methyl]-6,6- dimethyl-5,6,7,8- tetrahydroquinazolin-4-yl}- 2,3,4,5-tetrahydro-1,4- benzoxazepin-7-yl)pyridin-2- amine |
| 510 | 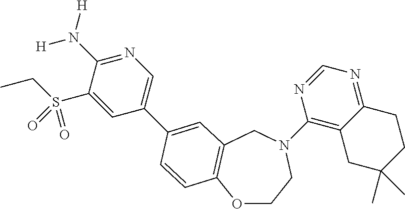 [SMILES: CCS(=O)(=O)c1cc(-c2ccc3c(c2)CN(c2ncnc4c2CC(C)(C)CC4)CCO3)cnc1N] | 5-[4-(6,6-dimethyl-5,6,7,8- tetrahydroquinazolin-4-yl)- 2,3,4,5-tetrahydro-1,4- benzoxazepin-7-yl]-3- (ethylsulfonyl)pyridin-2-amine |
| 511 | 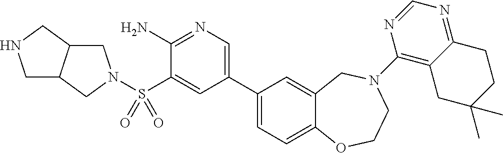 [SMILES: CC1(C)CCc2ncnc(N3CCOc4ccc(-c5cnc(N)c(S(=O)(=O)N6CC7CNCC7C6)c5)cc4C3)c2C1] | 5-[4-(6,6-dimethyl-5,6,7,8- tetrahydroquinazolin-4-yl)- 2,3,4,5-tetrahydro-1,4- benzoxazepin-7-yl]-3- (hexahydropyrrolo[3,4-c]pyrrol- 2(1H)-ylsulfonyl)pyridin-2- amine |
| 512 | 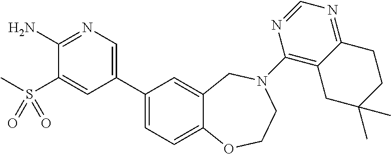 | 5-[4-(6,6-dimethyl-5,6,7,8- tetrahydroquinazolin-4-yl)- 2,3,4,5-tetrahydro-1,4- benzoxazepin-7-yl]-3- (methylsulfonyl)pyridin-2-amine |
| 513 | 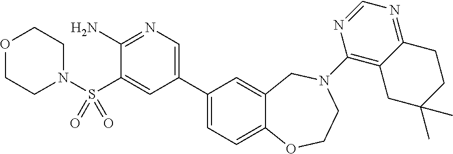 [SMILES: CC1(C)CCc2ncnc(N3CCOc4ccc(-c5cnc(N)c(S(=O)(=O)N6CCOCC6)c5)cc4C3)c2C1] | 5-[4-(6,6-dimethyl-5,6,7,8- tetrahydroquinazolin-4-yl)- 2,3,4,5-tetrahydro-1,4- benzoxazepin-7-yl]-3- (morpholin-4-ylsulfonyl)pyridin- 2-amine |
| 514 | 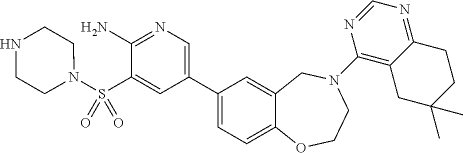 [SMILES: CC1(C)CCc2ncnc(N3CCOc4ccc(-c5cnc(N)c(S(=O)(=O)N6CCNCC6)c5)cc4C3)c2C1] | 5-[4-(6,6-dimethyl-5,6,7,8- tetrahydroquinazolin-4-yl)- 2,3,4,5-tetrahydro-1,4- benzoxazepin-7-yl]-3- (piperazin-1-ylsulfonyl)pyridin- 2-amine |
| 515 | 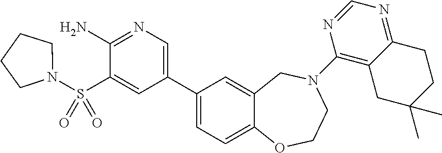 [SMILES: CC1(C)CCc2ncnc(N3CCOc4ccc(-c5cnc(N)c(S(=O)(=O)N6CCCC6)c5)cc4C3)c2C1] | 5-[4-(6,6-dimethyl-5,6,7,8- tetrahydroquinazolin-4-yl)- 2,3,4,5-tetrahydro-1,4- benzoxazepin-7-yl]-3- (piperazin-1-ylsulfonyl)pyridin- 2-amine |
| 516 | 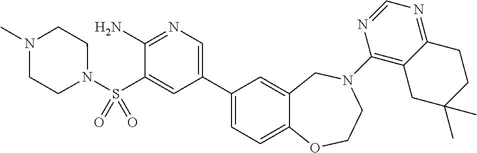 | 5-[4-(6,6-dimethyl-5,6,7,8- tetrahydroquinazolin-4-yl)- 2,3,4,5-tetrahydro-1,4- benzoxazepin-7-yl]-3-[(4- methylpiperazin-1- yl)sulfonyl]pyridin-2-amine |
| 517 | 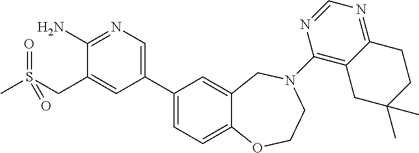 | 5-[4-(6,6-dimethyl-5,6,7,8- tetrahydroquinazolin-4-yl)- 2,3,4,5-tetrahydro-1,4- benzoxazepin-7-yl]-3- [(methylsulfonyl)methyl]pyridin- 2-amine |
| 518 | 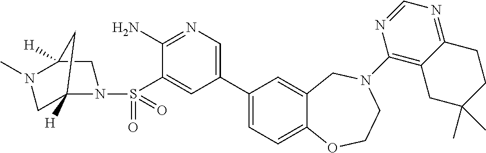 | 5-[4-(6,6-dimethyl-5,6,7,8- tetrahydroquinazolin-4-yl)- 2,3,4,5-tetrahydro-1,4- benzoxazepin-7-yl]-3-{[(1S,4S)- 5-methyl-2,5- diazabicyclo[2.2.1]hept-2- yl]sulfonyl}pyridin-2-amine |
| 519 | 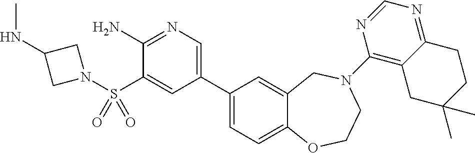 [SMILES: CNC1CN(S(=O)(=O)c2cc(-c3ccc4c(c3)CN(c3ncnc5c3CC(C)(C)CC5)CCO4)cnc2N)C1] | 5-[4-(6,6-dimethyl-5,6,7,8- tetrahydroquinazolin-4-yl)- 2,3,4,5-tetrahydro-1,4- benzoxazepin-7-yl]-3-{[3- (methylamino)azetidin-1- yl]sulfonyl}pyridin-2-amine |
| 520 | 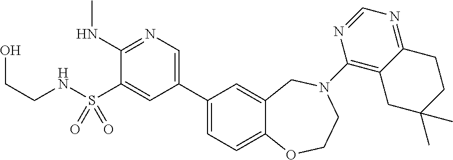 [SMILES: CNc1ncc(-c2ccc3c(c2)CN(c2ncnc4c2CC(C)(C)CC4)CCO3)cc1S(=O)(=O)NCCO] | 5-[4-(6,6-dimethyl-5,6,7,8- tetrahydroquinazolin-4-yl)- 2,3,4,5-tetrahydro-1,4- benzoxazepin-7-yl]-N-(2- hydroxyethyl)-2- (methylamino)pyridine-3- sulfonamide |
| 521 | 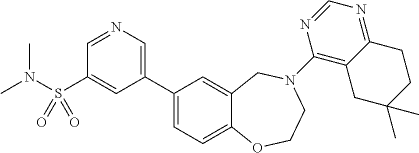 | 5-[4-(6,6-dimethyl-5,6,7,8- tetrahydroquinazolin-4-yl)- 2,3,4,5-tetrahydro-1,4- benzoxazepin-7-yl]-N,N- dimethylpyridine-3-sulfonamide |
| 522 | 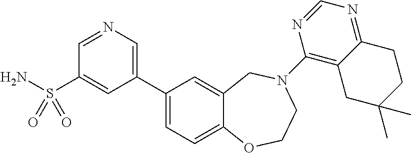 [SMILES: CC1(C)CCc2ncnc(N3CCOc4ccc(-c5cncc(S(N)(=O)=O)c5)cc4C3)c2C1] | 5-[4-(6,6-dimethyl-5,6,7,8- tetrahydroquinazolin-4-yl)- 2,3,4,5-tetrahydro-1,4- benzoxazepin-7-yl]pyridine-3- sulfonamide |
| 523 | 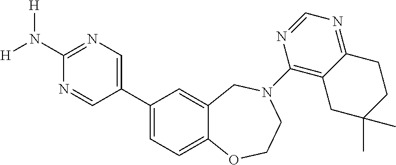 [SMILES: CC1(C)CCc2ncnc(N3CCOc4ccc(-c5cnc(N)nc5)cc4C3)c2C1] | 5-[4-(6,6-dimethyl-5,6,7,8- tetrahydroquinazolin-4-yl)- 2,3,4,5-tetrahydro-1,4- benzoxazepin-7-yl]pyrimidin-2- amine |
| 524 | 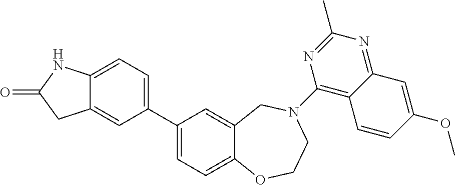 [SMILES: COc1ccc2c(N3CCOc4ccc(-c5ccc6c(c5)CC(=O)N6)cc4C3)nc(C)nc2c1] | 5-{4-[2-methyl-7- (methyloxy)quinazolin-4-yl]- 2,3,4,5-tetrahydro-1,4- benzoxazepin-7-yl}-1,3-dihydro- 2H-indol-2-one |
| 525 | 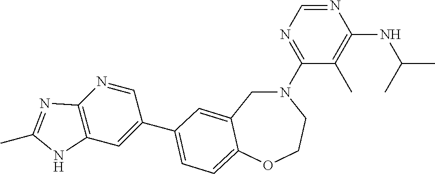 [SMILES: Cc1nc2ncc(-c3ccc4c(c3)CN(c3ncnc(NC(C)C)c3C)CCO4)cc2[nH]1] | 5-methyl-N-(1-methylethyl)-6- [7-(2-methyl-1H-imidazo[4,5- b]pyridin-6-yl)-2,3-dihydro-1,4- benzoxazepin-4(5H)- yl]pyrimidin-4-amine |
| 526 | 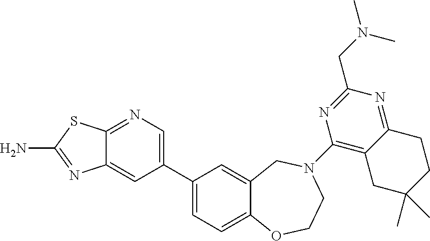 [SMILES: CN(C)Cc1nc2c(c(N3CCOc4ccc(-c5cnc6sc(N)nc6c5)cc4C3)n1)CC(C)(C)CC2] | 6-(4-{2- ((dimethylamino)methyl]-6,6- dimethyl-5,6,7,8- tetrahydroquinazolin-4-yl}- 2,3,4,5-tetrahydro-1,4- benzoxazepin-7- yl)[1,3]thiazolo[5,4-b]pyridin-2- amine |
| 527 | 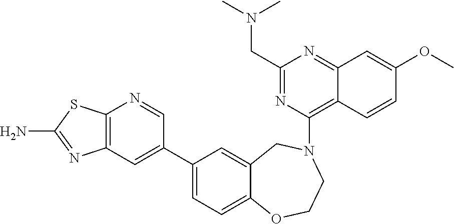 [SMILES: COc1ccc2c(N3CCOc4ccc(-c5cnc6sc(N)nc6c5)cc4C3)nc(CN(C)C)nc2c1] | 6-(4-{2- [(dimethylamino)methyl]-7- (methyloxy)quinazolin-4-yl}-2,3,4,5-tetrahydro-1,4-benzoxazepin-7-yl)[1,3]thiazolo[5,4-b]pyridin-2-amine |
| 529 | 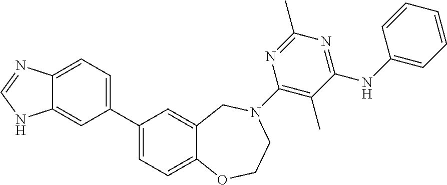 [SMILES: Cc1nc(Nc2ccccc2)c(C)c(N2CCOc3ccc(-c4ccc5nc[nH]c5c4)cc3C2)n1] | 6-[7-(1H-benzimidazol-6-yl)- 2,3-dihydro-1,4-benzoxazepin- 4(5H)-yl]-2,5-dimethyl-N- phenylpyrimidin-4-amine |
| 530 | 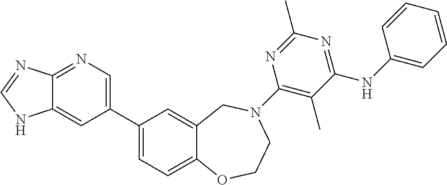 [SMILES: Cc1nc(Nc2ccccc2)c(C)c(N2CCOc3ccc(-c4cnc5nc[nH]c5c4)cc3C2)n1] | 6-[7-(1H-imidazo[4,5-b]pyridin- 6-yl)-2,3-dihydro-1,4- benzoxazepin-4(5H)-yl]-2,5- dimethyl-N-phenylpyrimidin-4- amine |
| 531 | 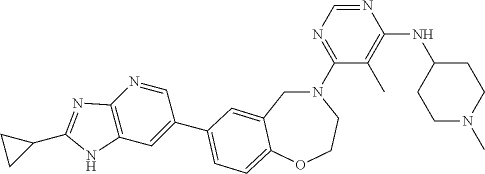 [SMILES: Cc1c(NC2CCN(C)CC2)ncnc1N1CCOc2ccc(-c3cnc4nc(C5CC5)[nH]c4c3)cc2C1] | 6-[7-(2-cyclopropyl-1H- imidazo[4,5-b]pyridin-6-yl)-2,3- dihydro-1,4-benzoxazepin- 4(5H)-yl]-5-methyl-N-(1- methylpiperidin-4-yl)pyrimidin- 4-amine |
| 532 | 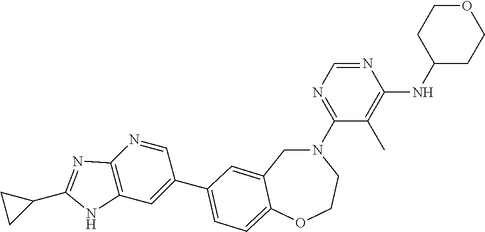 [SMILES: Cc1c(NC2CCOCC2)ncnc1N1CCOc2ccc(-c3cnc4nc(C5CC5)[nH]c4c3)cc2C1] | 6-[7-(2-cyclopropyl-1H- imidazo[4,5-b]pyridin-6-yl)-2,3- dihydro-1,4-benzoxazepin- 4(5H)-yl]-5-methyl-N- (tetrahydro-2H-pyran-4- yl)pyrimidin-4-amine |
| 533 | 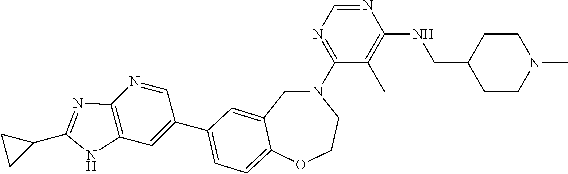 [SMILES: Cc1c(NCC2CCN(C)CC2)ncnc1N1CCOc2ccc(-c3cnc4nc(C5CC5)[nH]c4c3)cc2C1] | 6-[7-(2-cyclopropyl-1H- imidazo[4,5-b]pyridin-6-yl)-2,3- dihydro-1,4-benzoxazepin- 4(5H)-yl]-5-methyl-N-[(1- methylpiperidin-4- yl)methyl]pyrimidin-4-amine |
| 534 | 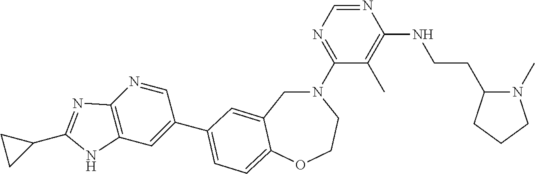 [SMILES: Cc1c(NCCC2CCCN2C)ncnc1N1CCOc2ccc(-c3cnc4nc(C5CC5)[nH]c4c3)cc2C1] | 6-[7-(2-cyclopropyl-1H- imidazo[4,5-b]pyridin-6-yl)-2,3- dihydro-1,4-benzoxazepin- 4(5H)-yl]-5-methyl-N-[2-(1- methylpyrrolidin-2- yl)ethyl]pyrimidin-4-amine |
| 535 | 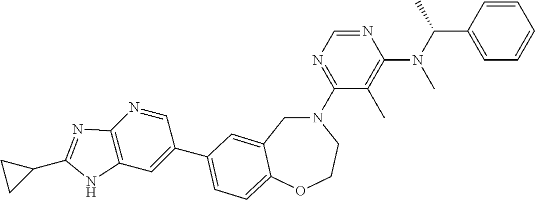 [SMILES: Cc1c(N2CCOc3ccc(-c4cnc5nc(C6CC6)[nH]c5c4)cc3C2)ncnc1N(C)[C@H](C)c1ccccc1] | 6-[7-(2-cyclopropyl-1H- imidazo[4,5-b]pyridin-6-yl)-2,3- dihydro-1,4-benzoxazepin- 4(5H)-yl]-N,5-dimethyl-N[(1R)- 1-phenylethyl]pyrimidin-4- amine |
| 536 | 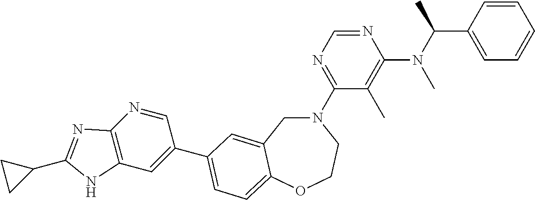 [SMILES: Cc1c(N2CCOc3ccc(-c4cnc5nc(C6CC6)[nH]c5c4)cc3C2)ncnc1N(C)[C@@H](C)c1ccccc1] | 6-[7-(2-cyclopropyl-1H- imidazo[4,5-b]pyridin-6-yl)-2,3- dihydro-1,4-benzoxazepin- 4(5H)-yl]-N,5-dimethyl-N-[(1S)- 1-phenylethyl]pyrimidin-4- amine |
| 537 | 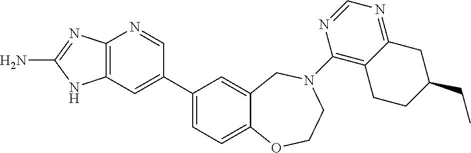 [SMILES: CC[C@H]1CCc2c(ncnc2N2CCOc3ccc(-c4cnc5nc(N)[nH]c5c4)cc3C2)C1] | 6-{4-[(7S)-7-ethyl-5,6,7,8- tetrahydroquinazolin-4-yl]- 2,3,4,5-tetrahydro-1,4- benzoxazepin-7-yl}-1H- imidazo[4,5-b]pyridin-2-amine |
| 538 | 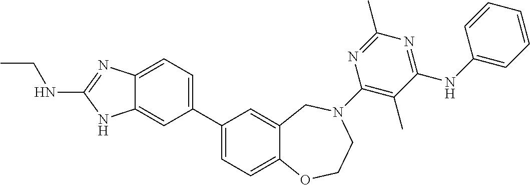 [SMILES: CCNc1nc2ccc(-c3ccc4c(c3)CN(c3nc(C)nc(Nc5ccccc5)c3C)CCO4)cc2[nH]1] | 6-{4-[2,5-dimethyl-6- (phenylamino)pyrimidin-4-yl]- 2,3,4,5-tetrahydro-1,4- benzoxazepin-7-yl}-N-ethyl-1H- benzimidazol-2-amine |
| 539 | 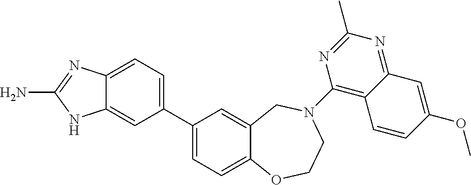 [SMILES: COc1ccc2c(N3CCOc4ccc(-c5ccc6nc(N)[nH]c6c5)cc4C3)nc(C)nc2c1] | 6-{4-[2-methyl-7- (methyloxy)quinazolin-4-yl]- 2,3,4,5-tetrahydro-1,4- benzoxazepin-7-yl}-1H- benzimidazol-2-amine |
| 540 | 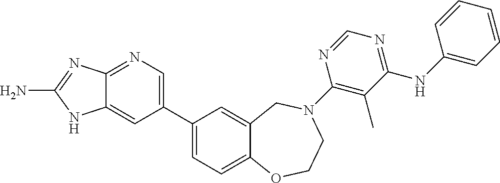 [SMILES: Cc1c(Nc2ccccc2)ncnc1N1CCOc2ccc(-c3cnc4nc(N)[nH]c4c3)cc2C1] | 6-{4-(5-methyl-6- (phenylamino)pyrimidin-4-yl]- 2,3,4,5-tetrahydro-1,4- benzoxazepin-7-yl}-1H- imidazo[4,5-b]pyridin-2-amine |
| 541 | 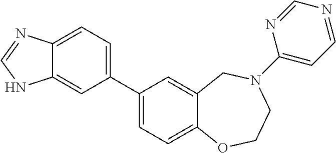 [SMILES: c1cc(N2CCOc3ccc(-c4ccc5nc[nH]c5c4)cc3C2)ncn1] | 7-(1H-benzimidazol-6-yl)-4- pyrimidin-4-yl-2,3,4,5- tetrahydro-1,4-benzoxazepine |
| 542 | 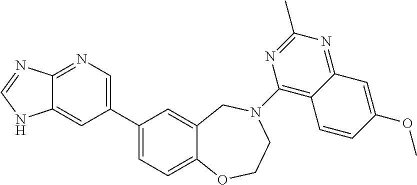 [SMILES: COc1ccc2c(N3CCOc4ccc(-c5cnc6nc[nH]c6c5)cc4C3)nc(C)nc2c1] | 7-(1H-imidazo[4,5-b]pyridin-6- yl)-4-(2-methyl-7- (methyloxy)quinazolin-4-yl]- 2,3,4,5-tetrahydro-1,4- benzoxazepine |
| 543 | 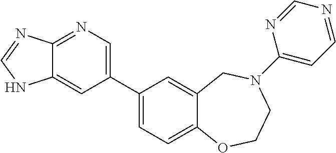 [SMILES: c1cc(N2CCOc3ccc(-c4cnc5nc[nH]c5c4)cc3C2)ncn1] | 7-(1H-imidazo[4,5-b]pyridin-6- yl)-4-pyrimidin-4-yl-2,3,4,5- tetrahydro-1,4-benzoxazepine |
| 544 | 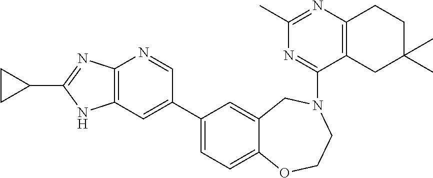 [SMILES: Cc1nc2c(c(N3CCOc4ccc(-c5cnc6nc(C7CC7)[nH]c6c5)cc4C3)n1)CC(C)(C)CC2] | 7-(2-cyclopropyl-1H- imidazo[4,5-b]pyridin-6-yl)-4- (2,6,6-trimethyl-5,6,7,8- tetrahydroquinazolin-4-yl)- 2,3,4,5-tetrahydro-1,4- benzoxazepine |
| 545 | 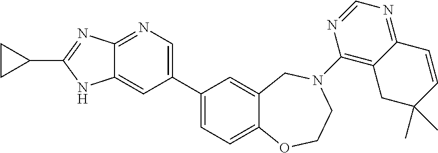 [SMILES: CC1(C)C=Cc2ncnc(N3CCOc4ccc(-c5cnc6nc(C7CC7)[nH]c6c5)cc4C3)c2C1] | 7-(2-cyclopropyl-1H- imidazo[4,5-b]pyridin-6-yl)-4- (6,6-dimethyl-5,6- dihydroquinazolin-4-yl)-2,3,4,5- tetrahydro-1,4-benzoxazepine |
| 546 | 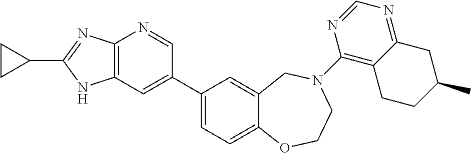 [SMILES: C[C@H]1CCc2c(ncnc2N2CCOc3ccc(-c4cnc5nc(C6CC6)[nH]c5c4)cc3C2)C1] | 7-(2-cydopropyl-1H- imidazo[4,5-b]pyridin-6-yl)-4- [(7S)-7-methyl-5,6,7,8- tetrahydroquinazolin-4-yl]- 2,3,4,5-tetrahydro-1,4- benzoxazepine |
| 547 | 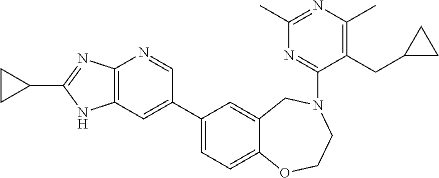 [SMILES: Cc1nc(C)c(CC2CC2)c(N2CCOc3ccc(-c4cnc5nc(C6CC6)[nH]c5c4)cc3C2)n1] | 7-(2-cyclopropyl-1H- imidazo[4,5-b]pyridin-6-yl)-4- [5-(cyclopropylmethyl)-2,6- dimethylpyrimidin-4-yl]-2,3,4,5- tetrahydro-1,4-benzoxazepine |
| 548 | 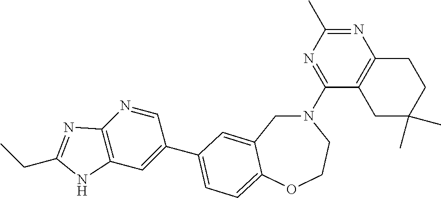 [SMILES: CCc1nc2ncc(-c3ccc4c(c3)CN(c3nc(C)nc5c3CC(C)(C)CC5)CCO4)cc2[nH]1] | 7-(2-ethyl-1H-imidazo[4,5- b]pyridin-6-yl)-4-(2,6,6- trimethyl-5,6,7,8- tetrahydroquinazolin-4-yl)- 2,3,4,5-tetrahydro-1,4- benzoxazepine |
| 550 | 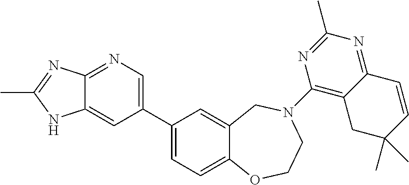 | 7-(2-methyl-1H-imidazo[4,5- b]pyridin-6-yl)-4-(2,6,6- trimethyl-5,6,7,8- tetrahydroquinazolin-4-yl)- 2,3,4,5-tetrahydro-1,4- benzoxazepine |
| 551 | 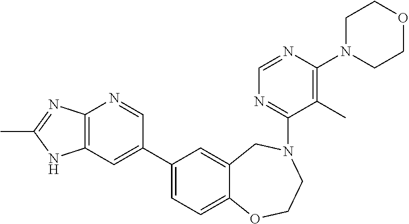 [SMILES: Cc1nc2ncc(-c3ccc4c(c3)CN(c3ncnc(N5CCOCC5)c3C)CCO4)cc2[nH]1] | 7-(2-methyl-1H-imidazo[4,5- b]pyridin-6-yl)-4-(5-methyl-6- morpholin-4-ylpyrimidin-4-yl)- 2,3,4,5-tetrahydro-1,4- benzoxazepine |
| 552 | 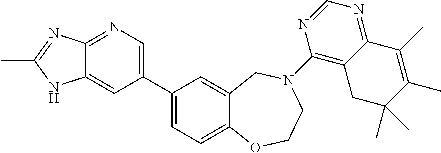 | 7-(2-methyl-1H-imidazo[4,5- b]pyridin-6-yl)-4-(6,6,7,8- tetramethyl-5,6- dihydroquinazolin-4-yl)-2,3,4,5- tetrahydro-1,4-benzoxazepine |
| 553 | 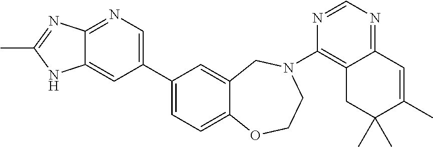 | 7-(2-methyl-1H-imidazo[4,5- b]pyridin-6-yl)-4-(6,6,7- trimethyl-5,6-dihydroquinazolin- 4-yl)-2,3,4,5-tetrahydro-1,4- benzoxazepine |
| 554 | 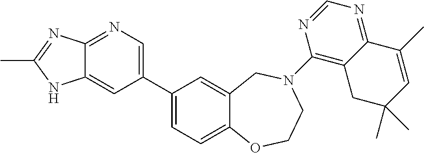 | 7-(2-methyl-1H-imidazo[4,5- b]pyridin-6-yl)-4-(6,6,8- trimethyl-5,6-dihydroquinazolin- 4-yl)-2,3,4,5-tetrahydro-1,4- benzoxazepine |
| 555 | 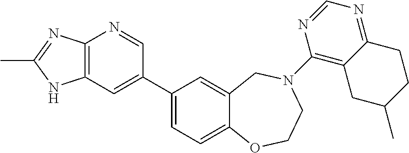 [SMILES: Cc1nc2ncc(-c3ccc4c(c3)CN(c3ncnc5c3CC(C)CC5)CCO4)cc2[nH]1] | 7-(2-methyl-1H-imidazo[4,5- b]pyridin-6-yl)-4-(6-methyl- 5,6,7,8-tetrahydroquinazolin-4- yl)-2,3,4,5-tetrahydro-1,4- benzoxazepine |
| 556 | 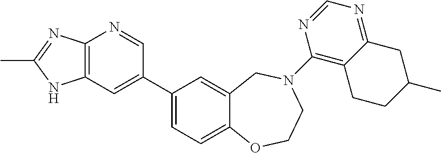 | 7-(2-methyl-1H-imidazo[4,5- b]pyridin-6-yl)-4-(7-methyl- 5,6,7,8-tetrahydroquinazolin-4- yl)-2,3,4,5-tetrahydro-1,4- benzoxazepine |
| 557 | 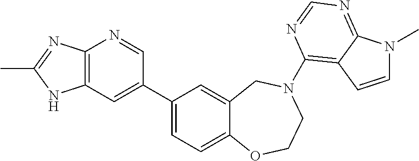 | 7-(2-methyl-1H-imidazo[4,5- b]pyridin-6-yl)-4-(7-methyl-7H- pyrrolo[2,3-d]pyrimidin-4-yl)- 2,3,4,5-tetrahydro-1,4- benzoxazepine |
| 558 | 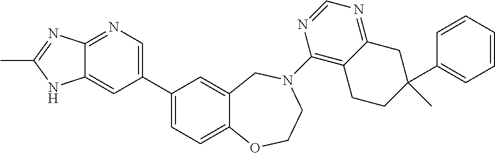 [SMILES: Cc1nc2ncc(-c3ccc4c(c3)CN(c3ncnc5c3CCC(C)(c3ccccc3)C5)CCO4)cc2[nH]1] | 7-(2-methyl-1H-imidazo[4,5- b]pyridin-6-yl)-4-(7-methyl-7- phenyl-5,6,7,8- tetrahydroquinazolin-4-yl)- 2,3,4,5-tetrahydro-1,4- benzoxazepine |
| 559 | 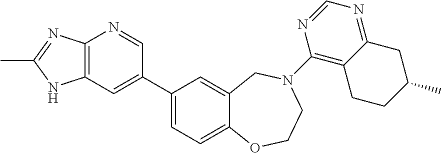 | 7-(2-methyl-1H-imidazo[4,5- b]pyridin-6-yl)-4-[(7R)-7- methyl-5,6,7,8- tetrahydroquinazolin-4-yl]- 2,3,4,5-tetrahydro-1,4- benzoxazepine |
| 560 | 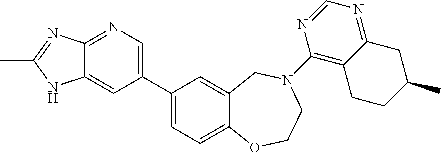 | 7-(2-methyl-1H-imidazo[4,5- b]pyridin-6-yl)-4-[(7S)-7- methyl-5,6,7,8- tetrahydroquinazolin-4-yl]- 2,3,4,5-tetrahydro-1,4- benzoxazepine |
| 561 | 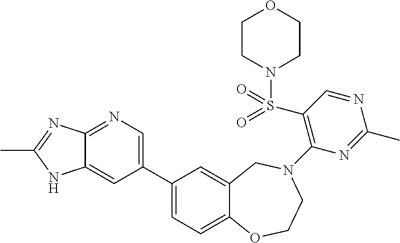 | 7-(2-methyl-1H-imidazo[4,5- b]pyridin-6-yl)-4-[2-methyl-5- (morpholin-4- ylsulfonyl)pyrimidin-4-yl]- 2,3,4,5-tetrahydro-1,4- benzoxazepine |
| 562 | 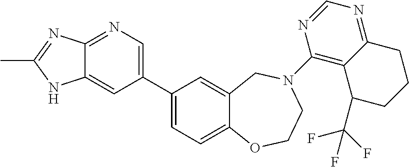 [SMILES: Cc1nc2ncc(-c3ccc4c(c3)CN(c3ncnc5c3C(C(F)(F)F)CCC5)CCO4)cc2[nH]1] | 7-(2-methyl-1H-imidazo[4,5- b]pyridin-6-yl)-4-[5- (trifluoromethyl)-5,6,7,8- tetrahydroquinazolin-4-yl]- 2,3,4,5-tetrahydro-1,4- benzoxazepine |
| 563 | 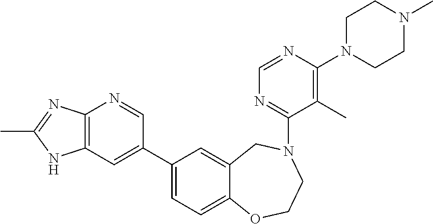 | 7-(2-methyl-1H-imidazo[4,5- b]pyridin-6-yl)-4-[5-methyl-6- (4-methylpiperazin-1- yl)pyrimidin-4-yl]-2,3,4,5- tetrahydro-1,4-benzoxazepine |
| 564 | 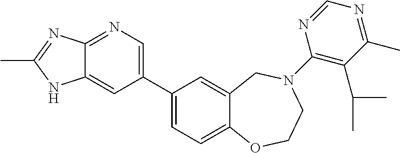 | 7-(2-methyl-1H-imidazo[4,5- b]pyridin-6-yl)-4-[6-methyl-5- (1-methylethyl)pyrimidin-4-yl]- 2,3,4,5-tetrahydro-1,4- benzoxazepine |
| 565 | 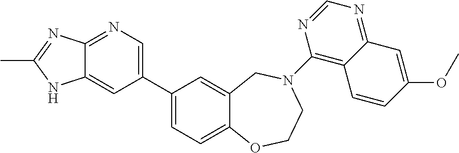 | 7-(2-methyl-1H-imidazo[4,5- b]pyridin-6-yl)-4-[7- (methyloxy)quinazolin-4-yl]- 2,3,4,5-tetrahydro-1,4- benzoxazepine |
| 566 | 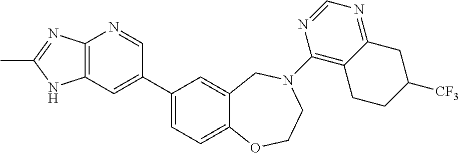 [SMILES: Cc1nc2ncc(-c3ccc4c(c3)CN(c3ncnc5c3CCC(C(F)(F)F)C5)CCO4)cc2[nH]1] | 7-(2-methyl-1H-imidazo[4,5- b]pyridin-6-yl)-4-[7- (trifluoromethyl)-5,6,7,8- tetrahydroquinazolin-4-yl]- 2,3,4,5-tetrahydro-1,4- benzoxazepine |
| 567 | 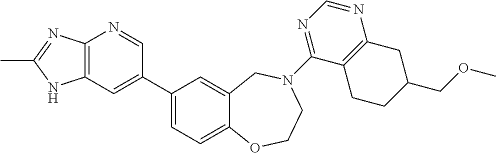 | 7-(2-methyl-1H-imidazo[4,5- b]pyridin-6-yl)-4-{7- [(methyloxy)methyl]-5,6,7,8- tetrahydroquinazolin-4-yl}- 2,3,4,5-tetrahydro-1,4- benzoxazepine |
| 568 | 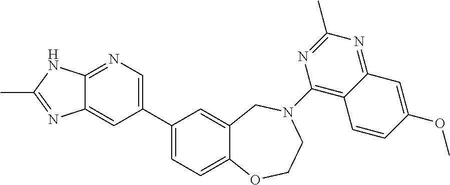 | 7-(2-methyl-3H-imidazo[4,5- b]pyridin-6-yl)-4-[2-methyl-7- (methyloxy)quinazolin-4-yl]- 2,3,4,5-tetrahydro-1,4- benzoxazepine |
| 569 | 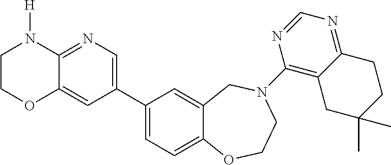 [SMILES: CC1(C)CCc2ncnc(N3CCOc4ccc(-c5cnc6c(c5)OCCN6)cc4C3)c2C1] | 7-(3,4-dihydro-2H-pyrido[3,2- b][1,4]oxazin-7-yl)-4-(6,6- dimethyl-5,6,7,8- tetrahydroquinazolin-4-yl)- 2,3,4,5-tetrahydro-1,4- benzoxazepine |
| 570 | 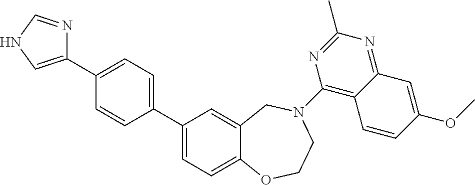 [SMILES: COc1ccc2c(N3CCOc4ccc(-c5ccc(-c6c[nH]cn6)cc5)cc4C3)nc(C)nc2c1] | 7-[4-(1H-imidazol-4-yl)phenyl]- 4-[2-methyl-7- (methyloxy)quinazolin-4-yl]- 2,3,4,5-tetrahydro-1,4- benzoxazepine |
| 571 | 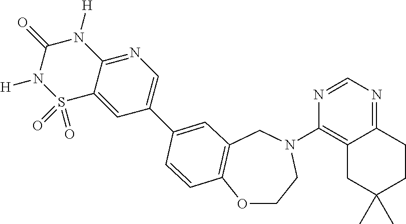 [SMILES: CC1(C)CCc2ncnc(N3CCOc4ccc(-c5cnc6c(c5)S(=O)(=O)NC(=O)N6)cc4C3)c2C1] | 7-[4-(6,6-dimethyl-5,6,7,8- tetrahydroquinazolin-4-yl)- 2,3,4,5-tetrahydro-1,4- benzoxazepin-7-yl]-2H- pyrido[2,3-e][1,2,4]thiadiazin- 3(4H)-one 1,1-dioxide |
| 572 | 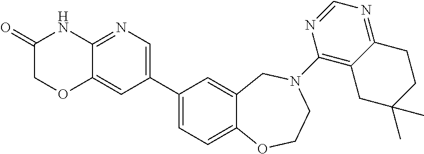 [SMILES: CC1(C)CCc2ncnc(N3CCOc4ccc(-c5cnc6c(c5)OCC(=O)N6)cc4C3)c2C1] | 7-[4-(6,6-dimethyl-5,6,7,8- tetrahydroquinazolin-4-yl)- 2,3,4,5-tetrahydro-1,4- benzoxazepin-7-yl]-2H- pyrido[3,2-b][1,4]oxazin-3(4H)- one |
| 573 | 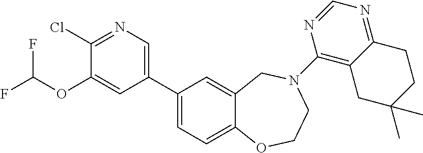 [SMILES: CC1(C)CCc2ncnc(N3CCOc4ccc(-c5cnc(Cl)c(OC(F)F)c5)cc4C3)c2C1] | 7-{6-chloro-5- [(difluoromethyl)oxy]pyridin-3- yl}-4-(6,6-dimethyl-5,6,7,8- tetrahydroquinazolin-4-yl)- 2,3,4,5-tetrahydro-1,4- benzoxazepine |
| 574 | 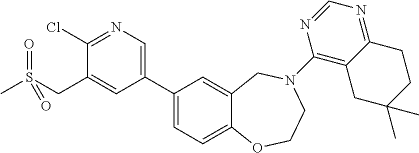 | 7-{6-chloro-5- [(methylsulfonyl)methyl]pyridin- 3-yl}-4-(6,6-dimethyl-5,6,7,8- tetrahydroquinazolin-4-yl)- 2,3,4,5-tetrahydro-1,4- benzoxazepine |
| 575 | 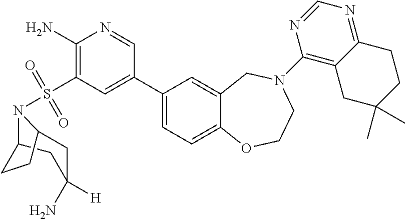 [SMILES: CC1(C)CCc2ncnc(N3CCOc4ccc(-c5cnc(N)c(S(=O)(=O)N6C7CCC6CC(N)C7)c5)cc4C3)c2C1] | 8-({2-amino-5-[4-(6,6-dimethyl- 5,6,7,8-tetrahydroquinazolin-4- yl)-2,3,4,5-tetrahydro-1,4- benzoxazepin-7-yl]pyridin-3- yl}sulfonyl)-8- azabicyclo[3.2.1]octan-3-amine |
| 576 | 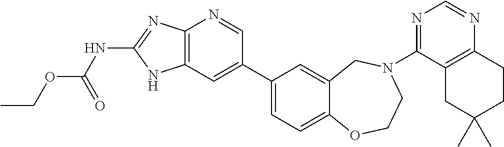 [SMILES: CCOC(=O)Nc1nc2ncc(-c3ccc4c(c3)CN(c3ncnc5c3CC(C)(C)CC5)CCO4)cc2[nH]1] | ethyl {6-[4-(6,6-dimethyl- 5,6,7,8-tetrahydroquinazolin-4- yl)-2,3,4,5-tetrahydro-1,4- benzoxazepin-7-yl]- 1H- imidazo[4,5-b]pyridin-2- yl}carbamate |
| 577 | 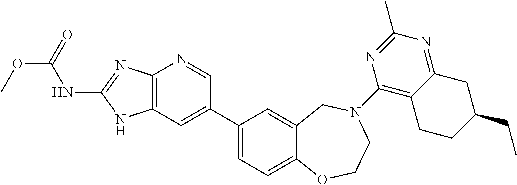 [SMILES: CC[C@H]1CCc2c(nc(C)nc2N2CCOc3ccc(-c4cnc5nc(NC(=O)OC)[nH]c5c4)cc3C2)C1] | methyl (6-{4-[(7S)-7-ethyl-2- methyl-5,6,7,8- tetrahydroquinazolin-4-yl]- 2,3,4,5-tetrahydro-1,4- benzoxazepin-7-yl}-1H- imidazo[4,5-b]pyridin-2- yl)carbamate |
| 578 | 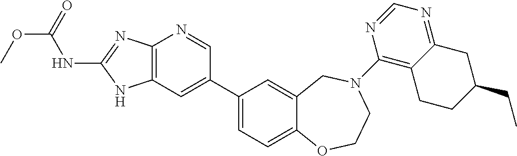 [SMILES: CC[C@H]1CCc2c(ncnc2N2CCOc3ccc(-c4cnc5nc(NC(=O)OC)[nH]c5c4)cc3C2)C1] | methyl (6-{4-[(7S)-7-ethyl- 5,6,7,8-tetrahydroquinazolin-4- yl]-2,3,4,5-tetrahydro-1,4- benzoxazepin-7-yl}-1H- imidazo[4,5-b] pyridin-2- yl)carbamate |
| 579 | 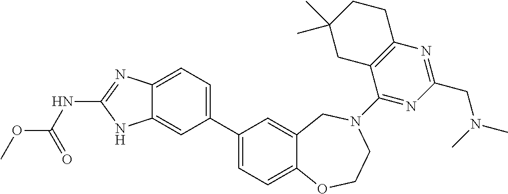 | methyl [6-(4-{2- [(dimethylamino)methyl]-6,6- dimethyl-5,6,7,8- tetrahydroquinazolin-4-yl}- 2,3,4,5-tetrahydro-1,4- benzoxazepin-7-yl)-1H- benzimidazol-2-yl]carbamate |
| 580 | 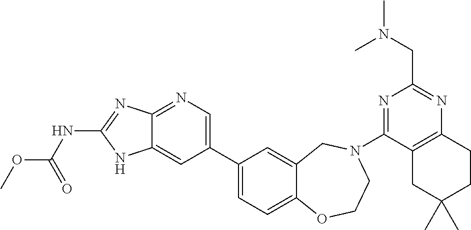 [SMILES: COC(=O)Nc1nc2ncc(-c3ccc4c(c3)CN(c3nc(CN(C)C)nc5c3CC(C)(C)CC5)CCO4)cc2[nH]1] | methyl [6-(4-{2- [(dimethylamino)methyl]-6,6- dimethyl-5,6,7,8- tetrahydroquinazolin-4-yl}- 2,3,4,5-tetrahydro-1,4- benzoxazepin-7-yl)-1H- imidazo[4,5-b]pyridin-2- yl]carbamate |
| 581 | 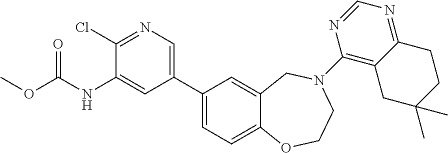 | methyl {2-chloro-5-[4-(6,6- dimethyl-5,6,7,8- tetrahydroquinazolin-4-yl)- 2,3,4,5-tetrahydro-1,4- benzoxazepin-7-yl]pyridin-3- yl}carbamate |
| 582 | 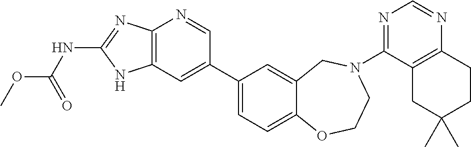 [SMILES: COC(=O)Nc1nc2ncc(-c3ccc4c(c3)CN(c3ncnc5c3CC(C)(C)CC5)CCO4)cc2[nH]1] | methyl {6-[4-(6,6-dimethyl- 5,6,7,8-tetrahydroquinazolin-4- yl)-2,3,4,5-tetrahydro-1,4- benzoxazepin-7-yl]-1H- imidazo[4,5-b]pyridin-2- yl}carbamate |
| 583 | 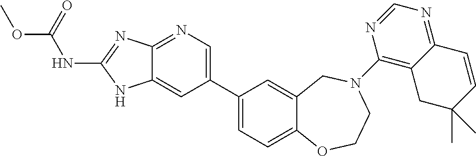 | methyl {6-[4-(6,6-dimethyl-5,6- dihydroquinazolin-4-yl)-2,3,4,5- tetrahydro-1,4-benzoxazepin-7- yl]-1H-imidazo[4,5-b]pyridin-2- yl}carbamate |
| 584 | 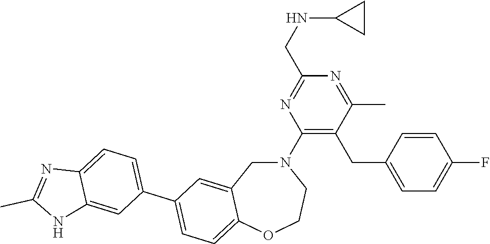 [SMILES: Cc1nc2ccc(-c3ccc4c(c3)CN(c3nc(CNC5CC5)nc(C)c3Cc3ccc(F)cc3)CCO4)cc2[nH]1] | N-({5-[(4-fluorophenyl)methyl]- 4-methyl-6-[7-(2-methyl-1H- benzimidazol-6-yl)-2,3-dihydro- 1,4-benzoxazepin-4(5H)- yl]pyrimidin-2- yl}methyl)cyclopropanamine |
| 585 | 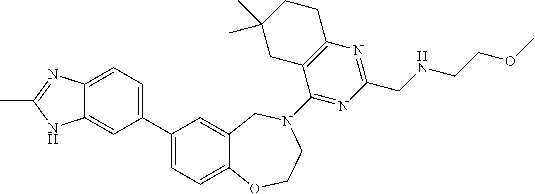 | N-({6,6-dimethyl-4-[7-(2- methyl-1H-benzimidazol-6-yl)- 2,3-dihydro-1,4-benzoxazepin- 4(5H)-yl]-5,6,7,8- tetrahydroquinazolin-2- yl}methyl)-2- (methyloxy)ethanamine |
| 586 | 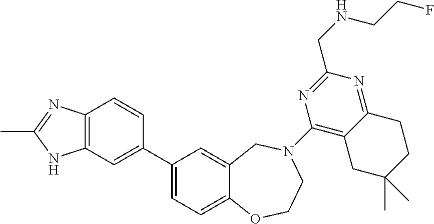 [SMILES: Cc1nc2ccc(-c3ccc4c(c3)CN(c3nc(CNCCF)nc5c3CC(C)(C)CC5)CCO4)cc2[nH]1] | N-({6,6-dimethyl-4-[7-(2- methyl-1H-benzimidazol-6-yl)- 2,3-dihydro-1,4-benzoxazepin- 4(5H)-yl]-5,6,7,8- tetrahydroquinazolin-2- yl}methyl)-2-fluoroethanamine |
| 587 | 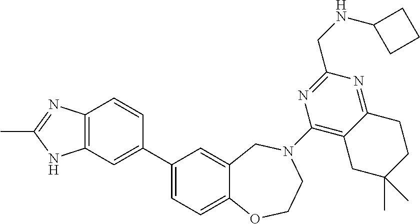 [SMILES: Cc1nc2ccc(-c3ccc4c(c3)CN(c3nc(CNC5CCC5)nc5c3CC(C)(C)CC5)CCO4)cc2[nH]1] | N-({6,6-dimethyl-4-[7-(2- methyl-1H-benzimidazol-6-yl)- 2,3-dihydro-1,4-benzoxazepin- 4(5H)-yl]-5,6,7,8- tetrahydroquinazolin-2- yl}methyl)cyclobutanamine |
| 588 | 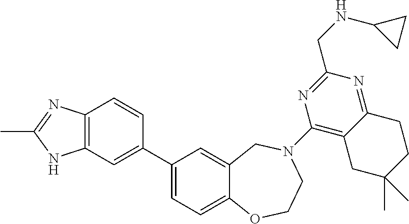 | N-({6,6-dimethyl-4-[7-(2- methyl-1H-benzimidazol-6-yl)- 2,3-dihydro-1,4-benzoxazepin- 4(5H)-yl]-5,6,7,8- tetrahydroquinazolin-2- yl}methyl)cyclopropanamine |
| 589 | 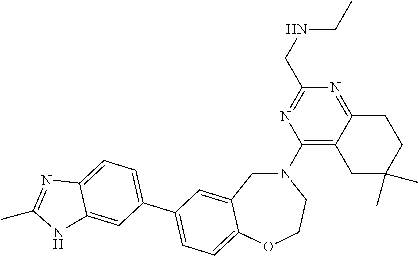 | N-({6,6-dimethyl-4-[7-(2- methyl-1H-benzimidazol-6-yl)- 2,3-dihydro-1,4-benzoxazepin- 4(5H)-yl]-5,6,7,8- tetrahydroquinazolin-2- yl}methyl)ethanamine |
| 590 | 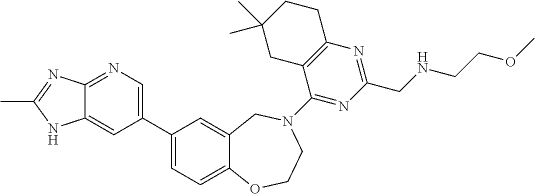 | N-({6,6-dimethyl-4-[7-(2- methyl-1H-imidazo[4,5- b]pyridin-6-yl)-2,3-dihydro-1,4- benzoxazepin-4(5H)-yl]-5,6,7,8- tetrahydroquinazolin-2- yl}methyl)-2- (methyloxy)ethanamine |
| 591 | 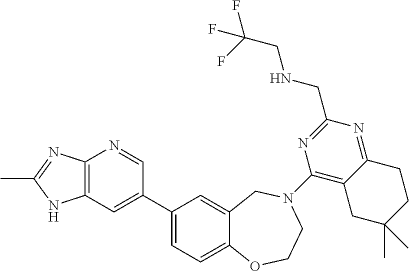 | N-({6,6-dimethyl-4-[7-(2- methyl-1H-imidazo[4,5- b]pyridin-6-yl)-2,3-dihydro-1,4- benzoxazepin-4(5H)-yl]-5,6,7,8- tetrahydroquinazolin-2- yl}methyl)-2,2,2- trifluoroethanamine |
| 592 | 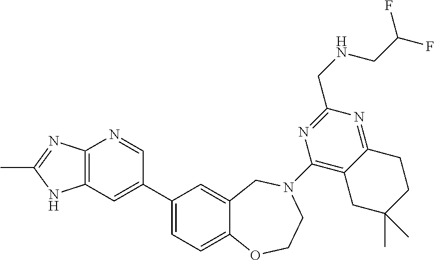 [SMILES: Cc1nc2ncc(-c3ccc4c(c3)CN(c3nc(CNCC(F)F)nc5c3CC(C)(C)CC5)CCO4)cc2[nH]1] | N-({6,6-dimethyl-4-[7-(2- methyl-1H-imidazo[4,5- b]pyridin-6-yl)-2,3-dihydro-1,4- benzoxazepin-4(5H)-yl]-5,6,7,8- tetrahydroquinazolin-2- yl}methyl)-2,2,- difluoroethanamine |
| 593 | 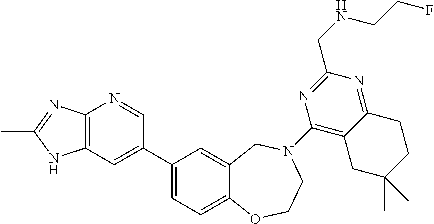 [SMILES: Cc1nc2ncc(-c3ccc4c(c3)CN(c3nc(CNCCF)nc5c3CC(C)(C)CC5)CCO4)cc2[nH]1] | N-({6,6-dimethyl-4-[7-(2- methyl-1H-imidazo[4,5- b]pyridin-6-yl)-2,3-dihydro-1,4- benzoxazepin-4(5H)-yl]-5,6,7,8- tetrahydroquinazolin-2- yl}methyl)-2-fluoroethanamine |
| 594 | 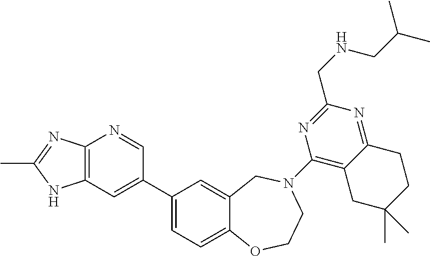 [SMILES: Cc1nc2ncc(-c3ccc4c(c3)CN(c3nc(CNCC(C)C)nc5c3CC(C)(C)CC5)CCO4)cc2[nH]1] | N-({6,6-dimethyl-4-[7-(2- methyl-1H-imidazo[4,5- b]pyridin-6-yl)-2,3-dihydro-1,4- benzoxazepin-4(5H)-yl]-5,6,7,8- tetrahydroquinazolin-2- yl}methyl)-2-methylpropan-1- amine |
| 595 | 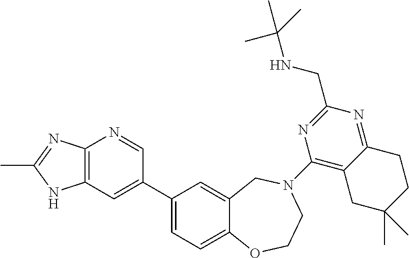 | N-({6,6-dimethyl-4-[7-(2- methyl-1H-imidazo[4,5- b]pyridin-6-yl)-2,3-dihydro-1,4- benzoxazepin-4(5H)-yl]-5,6,7,8- tetrahydroquinazolin-2- yl}methyl)-2-methylpropan-2- amine |
| 596 | 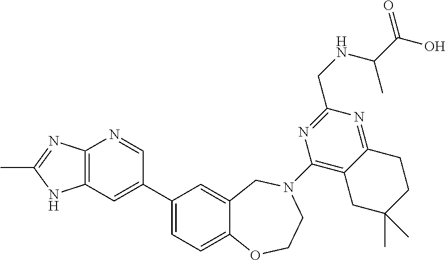 [SMILES: Cc1nc2ncc(-c3ccc4c(c3)CN(c3nc(CNC(C)C(=O)O)nc5c3CC(C)(C)CC5)CCO4)cc2[nH]1] | N-({6,6-dimethyl-4-[7-(2- methyl-1H-imidazo[4,5- b]pyridin-6-yl)-2,3-dihydro-1,4- benzoxazepin-4(5H)-yl]-5,6,7,8- tetrahydroquinazolin-2- yl}methyl)alanine |
| 597 | 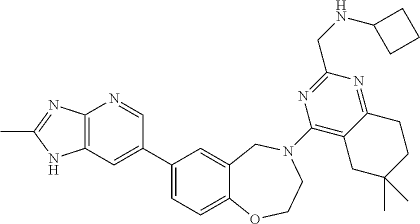 [SMILES: Cc1nc2ncc(-c3ccc4c(c3)CN(c3nc(CNC5CCC5)nc5c3CC(C)(C)CC5)CCO4)cc2[nH]1] | N-({6,6-dimethyl-4-[7-(2- methyl-1H-imidazo[4,5- b]pyridin-6-yl)-2,3-dihydro-1,4- benzoxazepin-4(5H)-yl]-5,6,7,8- tetrahydroquinazolin-2- yl}methyl)cyclobutanamine |
| 598 | 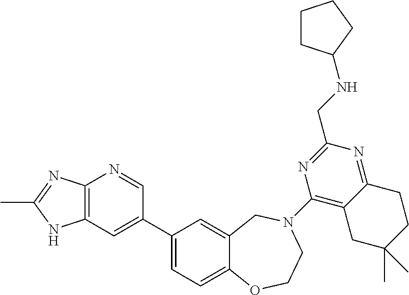 | N-({6,6-dimethyl-4-[7-(2- methyl-1H-imidazo[4,5- b]pyridin-6-yl)-2,3-dihydro-1,4- benzoxazepin-4(5H)-yl]-5,6,7,8- tetrahydroquinazolin-2- yl}methyl)cyclopentanamine |
| 599 | 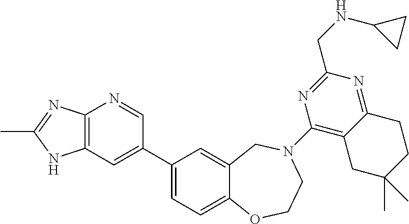 | N-({6,6-dimethyl-4-[7-(2- methyl-1H-imidazo[4,5- b]pyridin-6-yl)-2,3-dihydro-1,4- benzoxazepin-4(5H)-yl]-5,6,7,8- tetrahydroquinazolin-2- yl}methyl)cyclopropanamine |
| 600 | 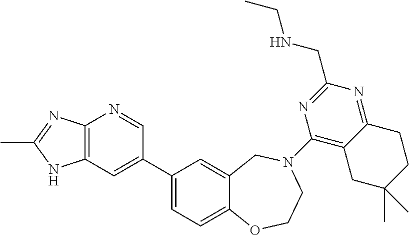 | N-({6,6-dimethyl-4-[7-(2- methyl-1H-imidazo[4,5- b]pyridin-6-yl)-2,3-dihydro-1,4- benzoxazepin-4(5H)-yl]-5,6,7,8- tetrahydroquinazolin-2- yl}methyl)ethanamine |
| 601 | 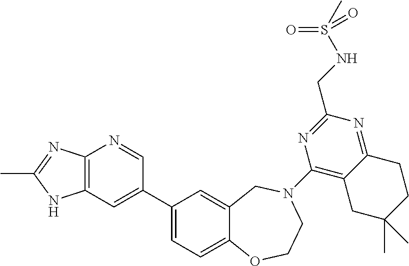 | N-({6,6-dimethyl-4-[7-(2- methyl-1H-imidazo[4,5- b]pyridin-6-yl)-2,3-dihydro-1,4- benzoxazepin-4(5H)-yl]-5,6,7,8- tetrahydroquinazolin-2- yl}methyl)methanesulfonamide |
| 602 | 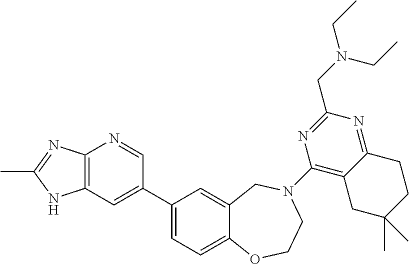 | N-({6,6-dimethyl-4-[7-(2- methyl-1H-imidazo[4,5- b]pyridin-6-yl)-2,3-dihydro-1,4- benzoxazepin-4(5H)-yl]-5,6,7,8- tetrahydroquinazolin-2- yl}methyl)-N-ethylethanamine |
| 603 | 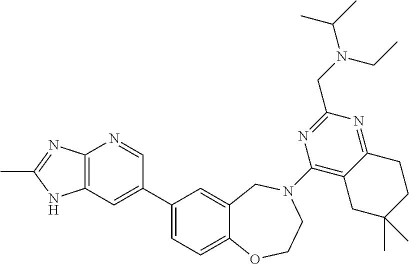 | N-({6,6-dimethyl-4-[7-(2- methyl-1H-imidazo[4,5- b]pyridin-6-yl)-2,3-dihydro-1,4- benzoxazepin-4(5H)-yl]-5,6,7,8- tetrahydroquinazolin-2- yl}methyl)-2-ethylpropan-2- amine |
| 604 | 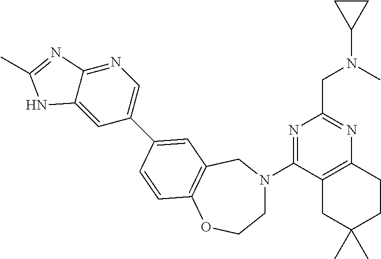 [SMILES: Cc1nc2ncc(-c3ccc4c(c3)CN(c3nc(CN(C)C5CC5)nc5c3CC(C)(C)CC5)CCO4)cc2[nH]1] | N-({6,6-dimethyl-4-[7-(2- methyl-1H-imidazo[4,5- b]pyridin-6-yl)-2,3-dihydro-1,4- benzoxazepin-4(5H)-yl]-5,6,7,8- tetrahydroquinazolin-2- yl}methyl)-N- methylcyclopropanamine |
| 605 | 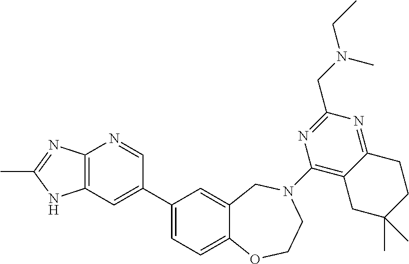 | N-({6,6-dimethyl-4-[7-(2- methyl-1H-imidazo[4,5- b]pyridin-6-yl)-2,3-dihydro-1,4- benzoxazepin-4(5H)-yl]-5,6,7,8- tetrahydroquinazolin-2- yl}methyl)-N-methylethanamine |
| 606 | 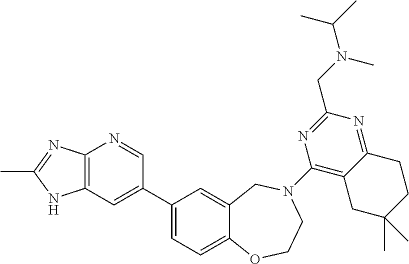 | N-({6,6-dimethyl-4-[7-(2- methyl-1H-imidazo[4,5- b]pyridin-6-yl)-2,3-dihydro-1,4- benzoxazepin-4(5H)-yl]-5,6,7,8- tetrahydroquinazolin-2- yl}methyl)-N-methylpropan-2- amine |
| 607 | 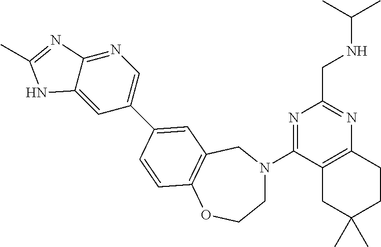 [SMILES: Cc1nc2ncc(-c3ccc4c(c3)CN(c3nc(CNC(C)C)nc5c3CC(C)(C)CC5)CCO4)cc2[nH]1] | N-({6,6-dimethyl-4-[7-(2- methyl-1H-imidazo[4,5- b]pyridin-6-yl)-2,3-dihydro-1,4- benzoxazepin-4(5H)-yl]-5,6,7,8- tetrahydroquinazolin-2- yl}methyl)propan-2-amine |
| 608 | 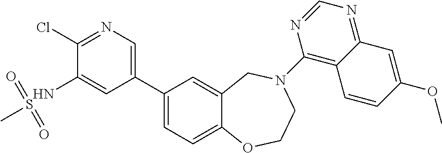 | N-(2-chloro-5-{4-[7- (methyloxy)quinazolin-4-yl]- 2,3,4,5-tetrahydro-1,4- benzoxazepin-7-yl}pyridin-3- yl)methanesulfonamide |
| 609 | 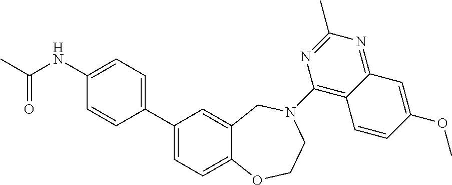 [SMILES: COc1ccc2c(N3CCOc4ccc(-c5ccc(NC(C)=O)cc5)cc4C3)nc(C)nc2c1] | N-(4-{4-[2-methyl-7- (methyloxy)quinazolin-4-yl]- 2,3,4,5-tetrahydro-1,4- benzoxazepin-7- yl}phenyl)acetamide |
| 610 | 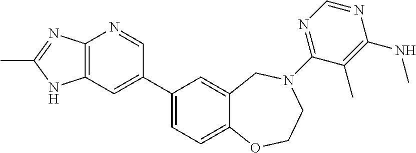 | N,5-dimethyl-6-[7-(2-methyl- 1H-imidazo[4,5-b]pyridin-6-yl)- 2,3-dihydro-1,4-benzoxazepin- 4(5H)-yl]pyrimidin-4-amine |
| 611 | 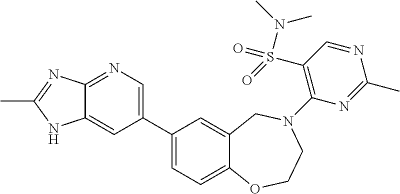 | N,N,2-trimethyl-4-[7-(2-methyl- 1H-imidazo[4,5-b]pyridin-6-yl)- 2,3-dihydro-1,4-benzoxazepin- 4(5H)-yl]pyrimidine-5- sulfonamide |
| 612 | 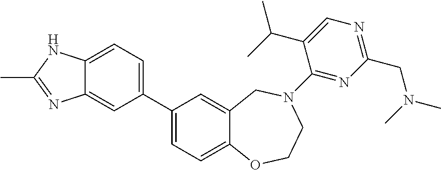 | N,N-dimethyl-1-{4-[7-(2- methyl-1H-benzimidazol-5-yl)- 2,3-dihydro-1,4-benzoxazepin- 4(5H)-yl]-5-(1- methylethyl)pyrimidin-2- yl}methanamine |
| 613 | 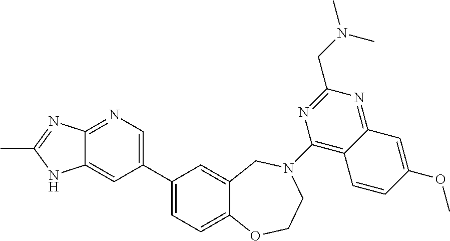 | N,N-dimethyl-1-{4-[7-(2- methyl-1H-imidazo[4,5- b]pyridin-6-yl)-2,3-dihydro-1,4- benzoxazepin-4(5H)-yl]-7- (methyloxy)quinazolin-2- yl}methanamine |
| 614 | 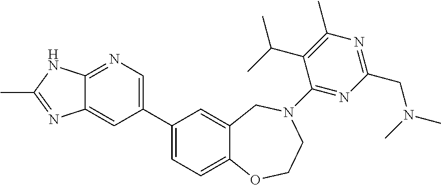 | N,N-dimethyl-1-{4-methyl-5-(1- methylethyl)-6-[7-(2-methyl-3H- imidazo[4,5-b]pyridin-6-yl)-2,3- dihydro-1,4-benzoxazepin- 4(5H)-yl]pyrimidin-2- yl}methanamine |
| 615 | 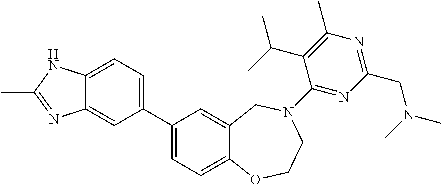 | N,N-dimethyl-1-{4-methyl-6-[7- (2-methyl-1H-benzimidazol-5- yl)-2,3-dihydro-1,4- benzoxazepin-4(5H)-yl]-5-(1- methylethyl)pyrimidin-2- yl}methanamine |
| 616 | 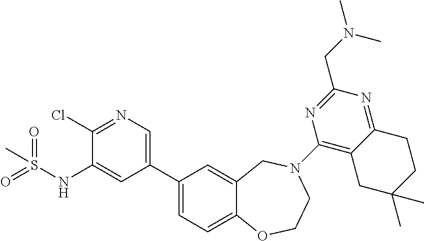 [SMILES: CN(C)Cc1nc2c(c(N3CCOc4ccc(-c5cnc(Cl)c(NS(C)(=O)=O)c5)cc4C3)n1)CC(C)(C)CC2] | N-[2-chloro-5-(4-{2- [(dimethylamino)methyl]-6,6- dimethyl-5,6,7,8- tetrahydroquinazolin-4-yl}- 2,3,4,5-tetrahydro-1,4- benzoxazepin-7-yl)pyridin-3- yl]methanesulfonamide |
| 617 | 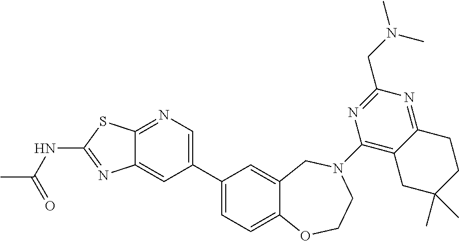 [SMILES: CC(=O)Nc1nc2cc(-c3ccc4c(c3)CN(c3nc(CN(C)C)nc5c3CC(C)(C)CC5)CCO4)cnc2s1] | N-[6-(4-{2- [(dimethylamino)methyl]-6,6- dimethyl-5,6,7,8- tetrahydroquinazolin-4-yl}- 2,3,4,5-tetrahydro-1,4- benzoxazepin-7- yl)[1,3]thiazolo[5,4-b]pyridin-2- yl]acetamide |
| 618 | 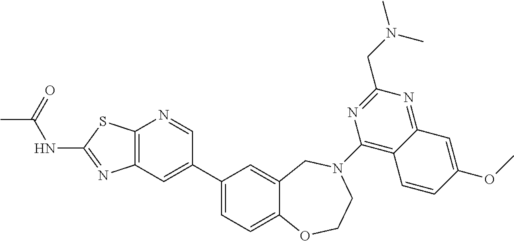 [SMILES: COc1ccc2c(N3CCOc4ccc(-c5cnc6sc(NC(C)=O)nc6c5)cc4C3)nc(CN(C)C)nc2c1] | N-[6-(4-{2- [(dimethylamino)methyl]-7- (methyloxy)quinazolin-4-yl}- 2,3,4,5-tetrahydro-1,4- benzoxazepin-7- yl)[1,3]thiazolo[5,4-b]pyridin-2- yl]acetamide |
| 619 | 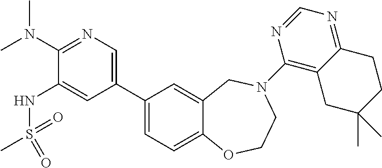 | N-{2-(dimethylamino)-5-[4-(6,6- dimethyl-5,6,7,8- tetrahydroquinazolin-4-yl)- 2,3,4,5-tetrahydro-1,4- benzoxazepin-7-yl]pyridin-3- yl}methanesulfonamide |
| 620 | 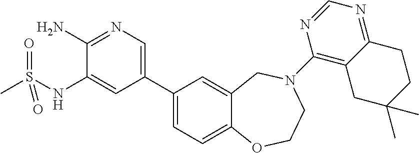 | N-{2-amino-5-[4-(6,6-dimethyl- 5,6,7,8-tetrahydroquinazolin-4- yl)-2,3,4,5-tetrahydro-1,4- benzoxazepin-7-yl]pyridin-3- yl}methanesulfonamide |
| 621 | 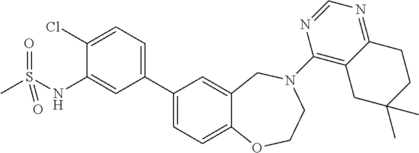 | N-{2-chloro-5-[4-(6,6-dimethyl- 5,6,7,8-tetrahydroquinazolin-4- yl)-2,3,4,5-tetrahydro-1,4- benzoxazepin-7- yl]phenyl}methanesulfonamide |
| 622 | 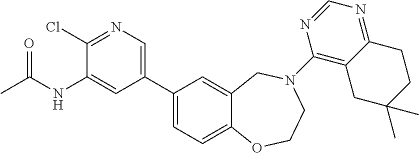 [SMILES: CC(=O)Nc1cc(-c2ccc3c(c2)CN(c2ncnc4c2CC(C)(C)CC4)CCO3)cnc1Cl] | N-{2-chloro-5-[4-(6,6-dimethyl- 5,6,7,8-tetrahydroquinazolin-4- yl)-2,3,4,5-tetrahydro-1,4- benzoxazepin-7-yl]pyridin-3- yl}acetamide |
| 623 | 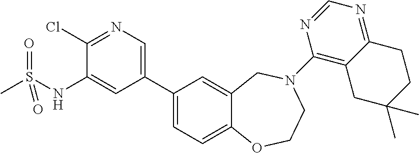 | N-{2-chloro-5-[4-(6,6-dimethyl-5,6,7,8-tetrahydroquinazolin-4-yl)-2,3,4,5-tetrahydro-1,4-benzoxazepin-7-yl]pyridin-3- yl}methanesulfonamide |
| 624 | 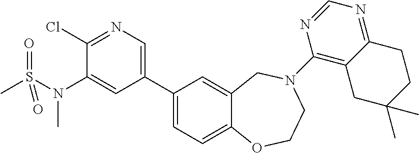 | N-{2-chloro-5-[4-(6,6-dimethyl- 5,6,7,8-tetrahydroquinazolin- 4-yl)-2,3,4,5-tetrahydro- 1,4-benzoxazepin-7-yl]pyridin- 3-yl}-N- methylmethanesulfonamide |
| 625 | 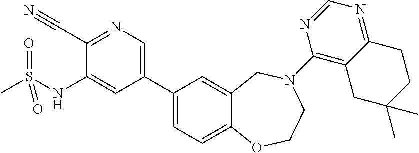 | N-{2-cyano-5-[4-(6,6-dimethyl- 5,6,7,8-tetrahydroquinazolin-4- yl)-2,3,4,5-tetrahydro-1,4- benzoxazepin-7-yl]pyridin-3- yl}methanesulfonamide |
| 626 | 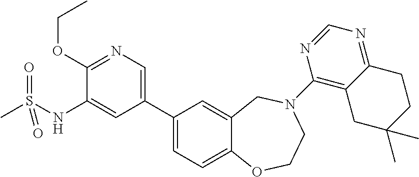 | N-{5-[4-(6,6-dimethyl-5,6,7,8- tetrahydroquinazolin-4-yl)- 2,3,4,5-tetrahydro-1,4- benzoxazepin-7-yl]-2- (ethyloxy)pyridin-3- yl}methanesulfonamide |
| 627 | 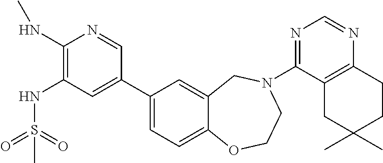 [SMILES: CNc1ncc(-c2ccc3c(c2)CN(c2ncnc4c2CC(C)(C)CC4)CCO3)cc1NS(C)(=O)=O] | N-{5-[4-(6,6-dimethyl-5,6,7,8- tetrahydroquinazolin-4-yl)- 2,3,4,5-tetrahydro-1,4- benzoxazepin-7-yl]-2- (methylamino)pyridin-3- yl} methanesulfonamide |
| 628 | 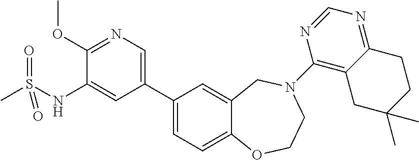 | N-{5-[4-(6,6-dimethyl-5,6,7,8- tetrahydroquinazolin-4-yl)- 2,3,4,5-tetrahydro-1,4- benzoxazepin-7-yl]-2- (methyloxy)pyridin-3- yl} methanesulfonamide |
| 629 | 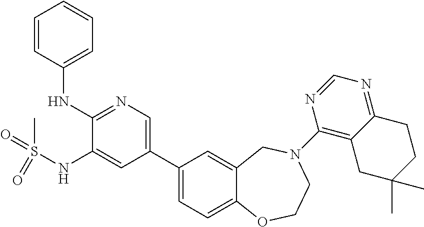 [SMILES: CC1(C)CCc2ncnc(N3CCOc4ccc(-c5cnc(Nc6ccccc6)c(NS(C)(=O)=O)c5)cc4C3)c2C1] | N-{5-[4-(6,6-dimethyl-5,6,7,8- tetrahydroquinazolin-4-yl)- 2,3,4,5-tetrahydro-1,4- benzoxazepin-7-yl]-2- (phenylamino)pyridin-3- yl}methanesulfonamide |
| 630 | 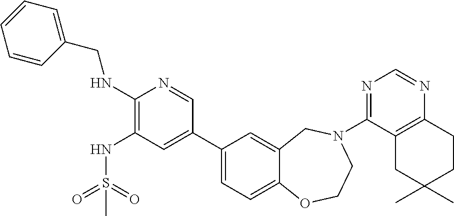 [SMILES: CC1(C)CCc2ncnc(N3CCOc4ccc(-c5cnc(NCc6ccccc6)c(NS(C)(=O)=O)c5)cc4C3)c2C1] | N-{5-[4-(6,6-dimethyl-5,6,7,8- tetrahydroquinazolin-4-yl)- 2,3,4,5-tetrahydro-1,4- benzoxazepin-7-yl]-2- [(phethylmethyl)amino]pyridin- 3-yl}methanesulfonamide |
| 631 | 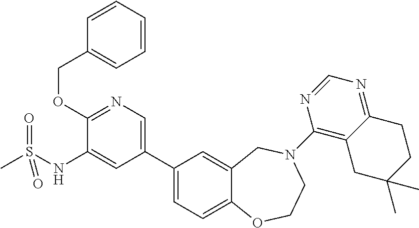 | N-{5-[4-(6,6-dimethyl-5,6,7,8- tetrahydroquinazolin-4-yl)- 2,3,4,5-tetrahydro-1,4- benzoxazepin-7-yl]-2- [(phenylmethyl)oxy]pyridin-3- yl}methanesulfonamide |
| 632 | 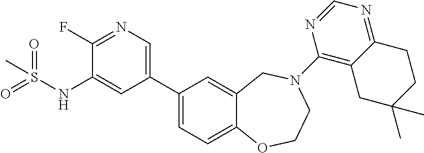 [SMILES: CC1(C)CCc2ncnc(N3CCOc4ccc(-c5cnc(F)c(NS(C)(=O)=O)c5)cc4C3)c2C1] | N-{5-[4-(6,6-dimethyl-5,6,7,8- tetrahydroquinazolin-4-yl)- 2,3,4,5-tetrahydro-1,4- benzoxazepin-7-yl]-2- fluoropyridin-3- yl}methanesulfonamide |
| 633 | 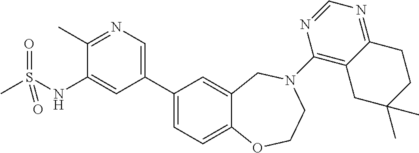 | N-{5-[4-(6,6-dimethyl-5,6,7,8- tetrahydroquinazolin-4-yl)- 2,3,4,5-tetrahydro-1,4- benzoxazepin-7-yl]-2- methylpyridin-3- yl}methanesulfonamide |
| 634 | 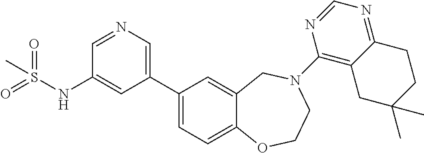 [SMILES: CC1(C)CCc2ncnc(N3CCOc4ccc(-c5cncc(NS(C)(=O)=O)c5)cc4C3)c2C1] | N-{5-[4-(6,6-dimethyl-5,6,7,8- tetrahydroquinazolin-4-yl)- 2,3,4,5-tetrahydro-1,4- benzoxazepin-7-yl]pyridin-3- yl}methanesulfonamide |
| 635 | 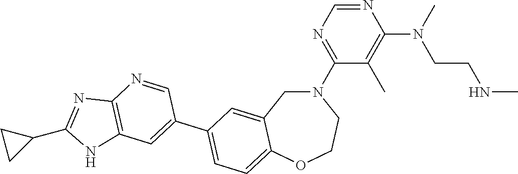 [SMILES: CNCCN(C)c1ncnc(N2CCOc3ccc(-c4cnc5nc(C6CC6)[nH]c5c4)cc3C2)c1C] | N-{6-[7-(2-cyclopropyl-1H- imidazo[4,5-b]pyridin-6-yl)-2,3- dihydro-1,4-benzoxazepin- 4(5H)-yl]-5-methylpyrimidin-4- yl}-N,N′-dimethylethane-1,2- diamine |
| 636 | 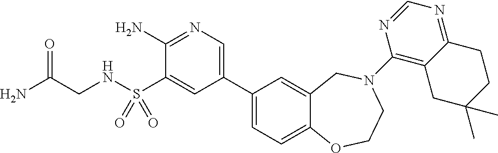 [SMILES: CC1(C)CCc2ncnc(N3CCOc4ccc(-c5cnc(N)c(S(=O)(=O)NCC(N)=O)c5)cc4C3)c2C1] | N~2~-({2-amino-5-[4-(6,6- dimethyl-5,6,7,8- tetrahydroquinazolin-4-yl)- 2,3,4,5-tetrahydro-1,4- benzoxazepin-7-yl]pyridin-3- yl}sulfonyl)glycinamide |
| 637 | 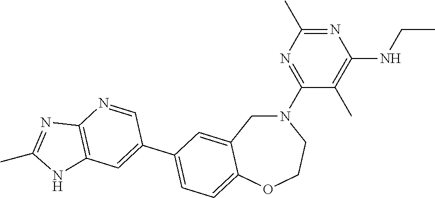 [SMILES: CCNc1nc(C)nc(N2CCOc3ccc(-c4cnc5nc(C)[nH]c5c4)cc3C2)c1C] | N-ethyl-2,5-dimethyl-6-[7-(2- methyl-1H-imidazo[4,5- b]pyridin-6-yl)-2,3-dihydro-1,4- benzoxazepin-4(5H)- yl]pyrimidin-4-amine |
| 638 | 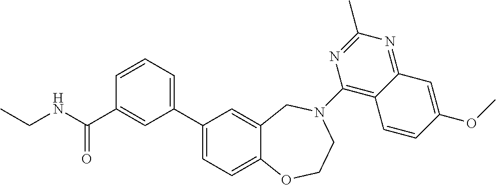 [SMILES: CCNC(=O)c1cccc(-c2ccc3c(c2)CN(c2nc(C)nc4cc(OC)ccc24)CCO3)c1] | N-ethyl-3-{4-[2-methyl-7- (methyloxy)quinazolin-4-yl]- 2,3,4,5-tetrahydro-1,4- benzoxazepin-7-yl}benzamide |
| 639 | 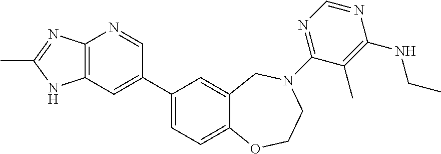 [SMILES: CCNc1ncnc(N2CCOc3ccc(-c4cnc5nc(C)[nH]c5c4)cc3C2)c1C] | N-ethyl-5-methyl-6-[7-(2- methyl-1H-imidazo[4,5- b]pyridin-6-yl)-2,3-dihydro-1,4- benzoxazepin-4(5H)- yl]pyrimidin-4-amine |
| 640 | 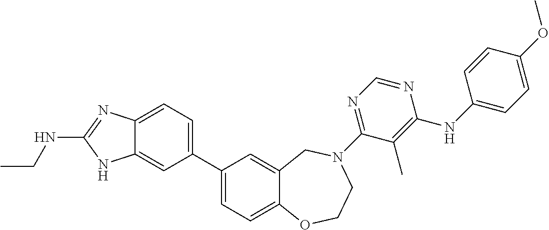 [SMILES: CCNc1nc2ccc(-c3ccc4c(c3)CN(c3ncnc(Nc5ccc(OC)cc5)c3C)CCO4)cc2[nH]1] | N-ethyl-6-[4-(5-methyl-6-{[4- (methyloxy)phenyl]amino}pyri- midin-4-yl)-2,3,4,5-tetrahydro- 1,4-benzoxazepin-7-yl]-1H- benzimidazol-2-amine |
| 641 | 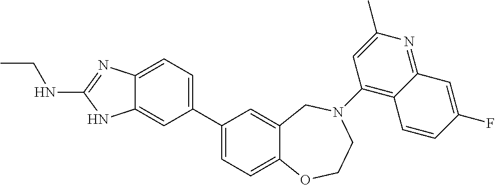 [SMILES: CCNc1nc2ccc(-c3ccc4c(c3)CN(c3cc(C)nc5cc(F)ccc35)CCO4)cc2[nH]1] | N-ethyl-6-[4-(7-fluoro-2- methylquinolin-4-yl)-2,3,4,5- tetrahydro-1,4-benzoxazepin-7- yl]-1H-benzimidazol-2-amine |
| 642 | 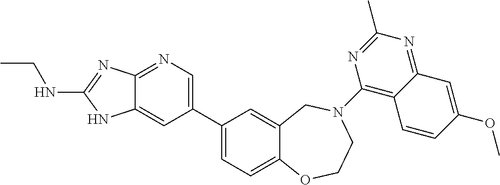 [SMILES: CCNc1nc2ncc(-c3ccc4c(c3)CN(c3nc(C)nc5cc(OC)ccc35)CCO4)cc2[nH]1] | N-ethyl-6-{4-[2-methyl-7- (methyloxy)quinazolin-4-yl]- 2,3,4,5-tetrahydro-1,4- benzoxazepin-7-yl}-1H- imidazo[4,5-b]pyridin-2-amine |
| 643 | 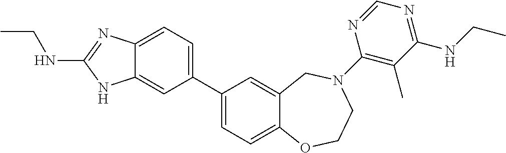 [SMILES: CCNc1nc2ccc(-c3ccc4c(c3)CN(c3ncnc(NCC)c3C)CCO4)cc2[nH]1] | N-ethyl-6-{4-[6-(ethylamino)-5- methylpyrimidin-4-yl]-2,3,4,5- tetrahydro-1,4-benzoxazepin-7- yl}-1H-benzimidazol-2-amine |
| 644 | 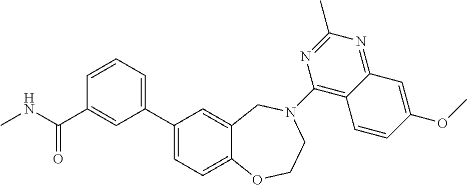 | N-methyl-3-{4-[2-methyl-7- (methyloxy)quinazolin-4-yl]- 2,3,4,5-tetrahydro-1,4- benzoxazepin-7-yl}benzamide |
| 645 | 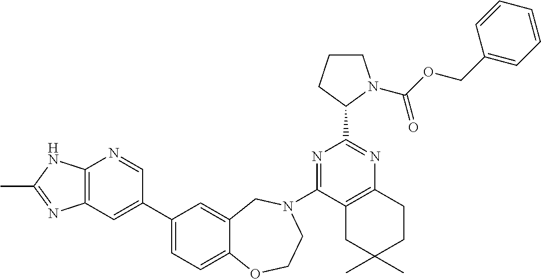 [SMILES: Cc1nc2cc(-c3ccc4c(c3)CN(c3nc([C@@H]5CCCN5C(=O)OCc5ccccc5)nc5c3CC(C)(C)CC5)CCO4)cnc2[nH]1] | phenylmethyl (2S)-2-{6,6- dimethyl-4-[7-(2-methyl-3H- imidazo[4,5-b]pyridin-6-yl)-2,3- dihydro-1,4-benzoxazepin- 4(5H)-yl]-5,6,7,8- tetrahydroquinazolin-2- yl}pyrrolidine-1-carboxylate |
| 646 | 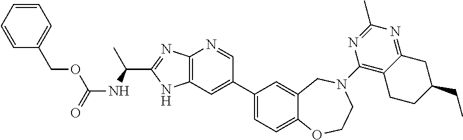 [SMILES: CC[C@H]1CCc2c(nc(C)nc2N2CCOc3ccc(-c4cnc5nc([C@H](C)NC(=O)OCc6ccccc6)[nH]c5c4)cc3C2)C1] | phenylmethyl [(1S)-1-(6-{4- [(7S)-7-ethyl-2-methyl-5,6,7,8- tetrahydroquinazolin-4-yl]- 2,3,4,5-tetrahydro-1,4- benzoxazepin-7-yl]-1H- imidazo[4,5-b]pyridin-2- yl)ethyl]carbamate |
| 647 | 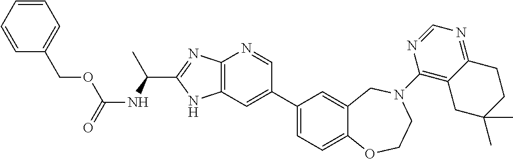 [SMILES: C[C@H](NC(=O)OCc1ccccc1)c1nc2ncc(-c3ccc4c(c3)CN(c3ncnc5c3CC(C)(C)CC5)CCO4)cc2[nH]1] | phenylmethyl [(1S)-1-{6-[4-(6,6- dimethyl-5,6,7,8- tetrahydroquinazolin-4-yl)- 2,3,4,5-tetrahydro-1,4- benzoxazepin-7-yl]-1H- imidazo[4,5-b]pyridin-2- yl}ethyl]carbamate |
| 648 | 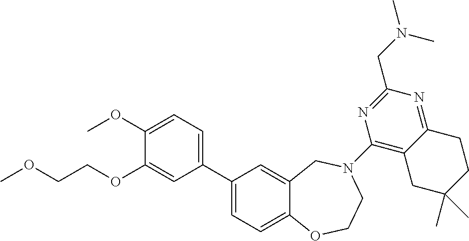 | 1-(6,6-dimethyl-4-{7-[4- (methyloxy)-3-{[2- (methyloxy)ethyl]oxy}phenyl]- 2,3-dihydro-1,4-benzoxazepin- 4(5H)-yl}-5,6,7,8- tetrahydroquinazolin-2-yl)-N,N- dimethylmethanamine |
| 649 | 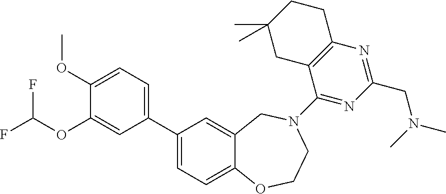 [SMILES: COc1ccc(-c2ccc3c(c2)CN(c2nc(CN(C)C)nc4c2CC(C)(C)CC4)CCO3)cc1OC(F)F] | l-{4-[7-{3- [(difluoromethyl)oxy]-4- (methyloxy)phenyl}-2,3- dihydro-1,4-benzoxazepin- 4(5H)-yl]-6,6-dimethyl-5,6,7,8- tetrahydroquinazolin-2-yl}-N,N- dimethylmethanamine |
| 650 | 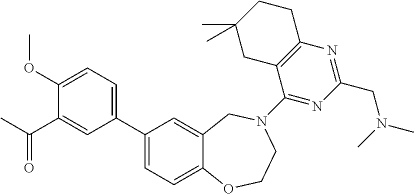 [SMILES: COc1ccc(-c2ccc3c(c2)CN(c2nc(CN(C)C)nc4c2CC(C)(C)CC4)CCO3)cc1C(C)=O] | l-[5-(4-{2- [(dimethylamino)methyl]-6,6- dimethyl-5,6,7,8- tetrahydroquinazolin-4-yl}- 2,3,4,5-tetrahydro-1,4- benzoxazepin-7-yl)-2- (methyloxy)phenyl]ethanone |
| 651 | 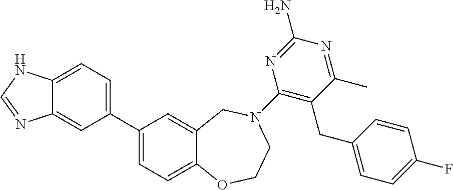 [SMILES: Cc1nc(N)nc(N2CCOc3ccc(-c4ccc5[nH]cnc5c4)cc3C2)c1Cc1ccc(F)cc1] | 4-[7-(1H-benzimidazol-5-yl)- 2,3-dihydro-1,4-benzoxazepin- 4(5H)-yl]-5-[(4- fluorophenyl)methyl]-6- methylpyrimidin-2-amine |
| 652 | 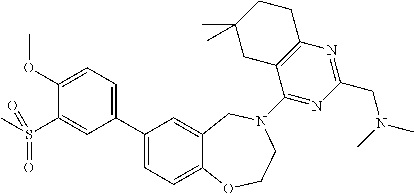 | 1-(6,6-dimethyl-4-{7-[4- (methyloxy)-3- (methylsulfonyl)phenyl]-2,3- dihydro-1,4-benzoxazepin- 4(5H)-yl}-5,6,7,8- tetrahydroquinazolin-2-yl)-N,N- dimethylmethanamine |
| 653 | 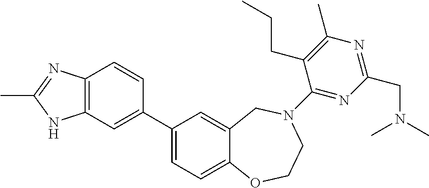 | N,N-dimethyl-1-{4-methyl-6-[7- (2-methyl-1H-benzimidazol-6- yl)-2,3-dihydro-1,4- benzoxazepin-4(5H)-yl]-5- propylpyrimidin-2- yl}methanamine |
| 654 | 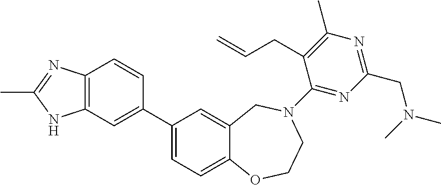 | N,N-dimethyl-1-{4-methyl-6-[7- (2-methyl-1H-benzimidazol-6- yl)-2,3-dihydro-1,4- benzoxazepin-4(5H)-yl]-5-prop 2-en-1-ylpyrimidin-2- yl}methanamine |
| 655 | 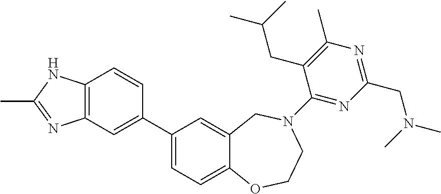 | N,N-dimethyl-1-{4-methyl-6-[7- (2-methyl-1H-benzimidazol-5- yl)-2,3-dihydro-1,4- benzoxazepin-4(5H)-yl]-5-(2- methylpropyl)pyrimidin-2- yl}methanamine |
| 656 | 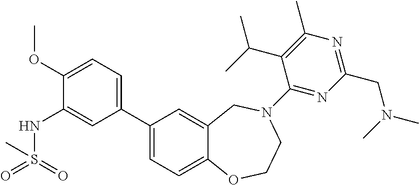 [SMILES: COc1ccc(-c2ccc3c(c2)CN(c2nc(CN(C)C)nc(C)c2C(C)C)CCO3)cc1NS(C)(=O)=O] | N-[5-(4-{2- [(dimethylamino)methyl]-6- methyl-5-(1- methylethyl)pyrimidin-4-yl}- 2,3,4,5-tetrahydro-1,4- benzoxazepin-7-yl)-2- (methyloxy)phenyl]methane- sulfonamide |
| 657 | 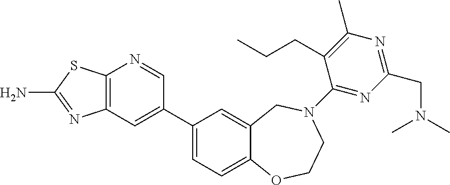 [SMILES: CCCc1c(C)nc(CN(C)C)nc1N1CCOc2ccc(-c3cnc4sc(N)nc4c3)cc2C1] | 6-(4-{2- [(dimethylamino)methyl]-6- methyl-5-propylpyrimidin-4-yl}- 2,3,4,5-tetrahydro-1,4- benzoxazepin-7- yl)[1,3]thiazolo[5,4-b]pyridin-2- amine |
| 658 | 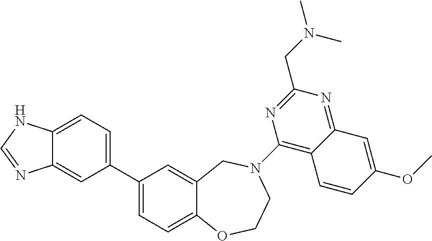 [SMILES: COc1ccc2c(N3CCOc4ccc(-c5ccc6[nH]cnc6c5)cc4C3)nc(CN(C)C)nc2c1] | 1-{4-[7-(1H-benzimidazol-5-yl)- 2,3-dihydro-1,4-benzoxazepin- 4(5H)-yl]-7- (methyloxy)quinazolin-2-yl}- N,N-dimethylmethanamine |
| 659 | 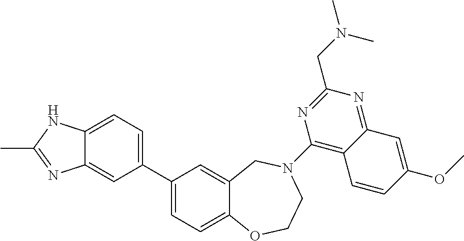 | N,N-dimethyl-1-{4-[7-(2- methyl-1H-benzimidazol-5-yl)- 2,3-dihydro-1,4-benzoxazepin- 4(5H)-yl]-7- (methyloxy)quinazolin-2- yl}methanamine |
| 660 | 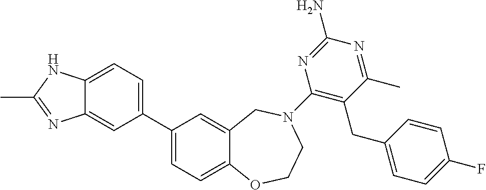 [SMILES: Cc1nc2cc(-c3ccc4c(c3)CN(c3nc(N)nc(C)c3Cc3ccc(F)cc3)CCO4)ccc2[nH]1] | 5-[(4-fluorophenyl)methyl]-4- methyl-6-[7-(2-methyl-1H- benzimidazol-5-yl)-2,3-dihydro- 1,4-benzoxazepin-4(5H)- yl]pyrimidin-2-amine |
| 661 | 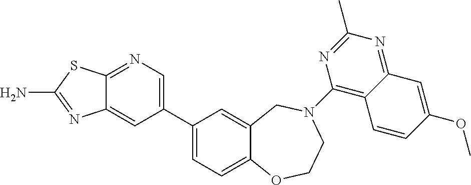 [SMILES: COc1ccc2c(N3CCOc4ccc(-c5cnc6sc(N)nc6c5)cc4C3)nc(C)nc2c1] | 6-{4-[2-methyl-7- (methyloxy)quinazolin-4-yl]- 2,3,4,5-tetrahydro-1,4- benzoxazepin-7- yl}[1,3]thiazolo[5,4-b]pyridin-2- amine |
| 662 | 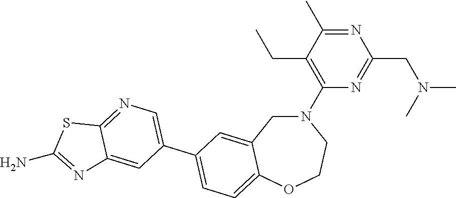 [SMILES: CCc1c(C)nc(CN(C)C)nc1N1CCOc2ccc(-c3cnc4sc(N)nc4c3)cc2C1] | 6-(4-{2- [(dimethylamino)methyl]-5- ethyl-6-methylpyrimidin-4-yl}- 2,3,4,5-tetrahydro-1,4- benzoxazepin-7- yl)[1,3]thiazolo[5,4-b]pyridin-2- amine |
| 663 | 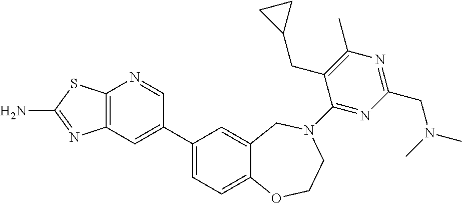 [SMILES: Cc1nc(CN(C)C)nc(N2CCOc3ccc(-c4cnc5sc(N)nc5c4)cc3C2)c1CC1CC1] | 6-(4-{5-(cyclopropylmethyl)-2- [(dimethylamino)methyl]-6- methylpyrimidin-4-yl}-2,3,4,5- tetrahydro-1,4-benzoxazepin-7- yl)[1,3]thiazolo[5,4-b]pyridin-2- amine |
| 664 | 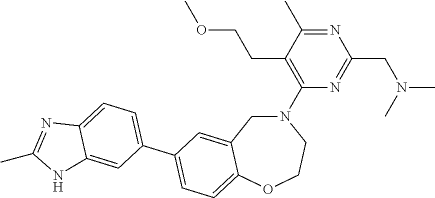 | N,N-dimethyl-1-{4-methyl-6-[7- (2-methyl-1H-benzimidazol-6- yl)-2,3-dihydro-1,4- benzoxazepin-4(5H)-yl]-5-[2- (methyloxy)ethyl]pyrimidin-2- yl}methanamine |
| 665 | 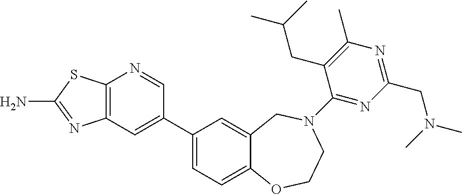 [SMILES: Cc1nc(CN(C)C)nc(N2CCOc3ccc(-c4cnc5sc(N)nc5c4)cc3C2)c1CC(C)C] | 6-(4-{2- [(dimethylamino)methyl]-6- methyl-5-(2- methylpropyl)pyrimidin-4-yl}- 2,3,4,5-tetrahydro-1,4- benzoxazepin-7- yl)[1,3]thiazolo[5,4-b]pyridin-2- amine |
| 666 | 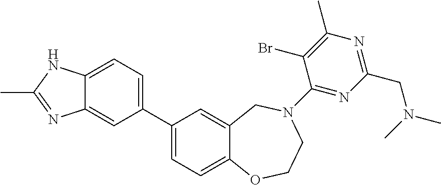 | 1-{5-bromo-4-methyl-6-[7-(2- methyl-1H-benzimidazol-5-yl)- 2,3-dihydro-1,4-benzoxazepin- 4(5H)-yl]pyrimidin-2-yl}-N,N- dimethylmethanamine |
| 667 | 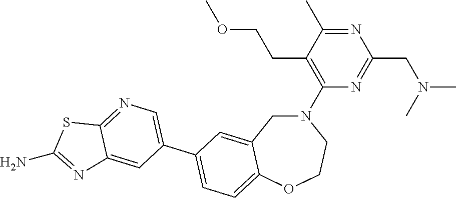 [SMILES: COCCc1c(C)nc(CN(C)C)nc1N1CCOc2ccc(-c3cnc4sc(N)nc4c3)cc2C1] | 6-(4-{2- [(dimethylamino)methyl]-6- methyl-5-[2- (methyloxy)ethyl]pyrimidin-4- yl}-2,3,4,5-tetrahydro-1,4- benzoxazepin-7- yl)[1,3]thiazolo[5,4-b]pyridin-2- amine |
| 668 | 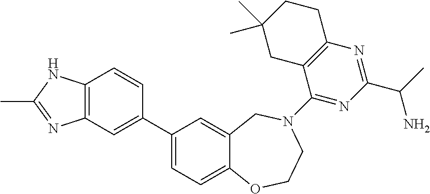 [SMILES: Cc1nc2cc(-c3ccc4c(c3)CN(c3nc(C(C)N)nc5c3CC(C)(C)CC5)CCO4)ccc2[nH]1] | l-{6,6-dimethyl-4-[7-(2-methyl- 1H-benzimidazol-5-yl)-2,3- dihydro-1,4-benzoxazepin- 4(5H)-yl]-5,6,7,8- tetrahydroquinazolin-2- yl}ethanamine |
| 669 | 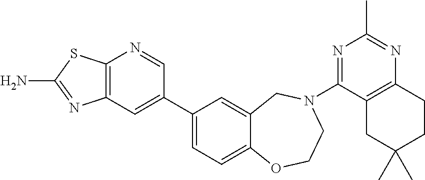 [SMILES: Cc1nc2c(c(N3CCOc4ccc(-c5cnc6sc(N)nc6c5)cc4C3)n1)CC(C)(C)CC2] | 6-[4-(2,6,6-trimethyl-5,6,7,8- tetrahydraquinazolin-4-yl)- 2,3,4,5-tetrahydro-1,4- benzoxazepin-7- yl][1,3]thiazolo[5,4-b]pyridin-2- amine |
| 670 | 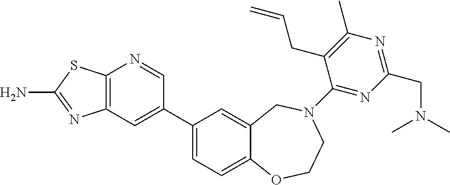 [SMILES: C=CCc1c(C)nc(CN(C)C)nc1N1CCOc2ccc(-c3cnc4sc(N)nc4c3)cc2C1] | 6-(4-{2- [(dimethylamino)methyl]-6- methyl-5-prop-2-en-1- ylpyrimidin-4-yl}-2,3,4,5- tetrahydro-1,4-benzoxazepin-7- yl)[1,3]thiazolo[5,4-b]pyridin-2- amine |
| 671 | 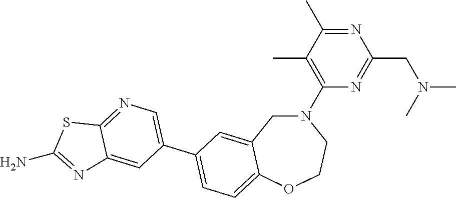 [SMILES: Cc1nc(CN(C)C)nc(N2CCOc3ccc(-c4cnc5sc(N)nc5c4)cc3C2)c1C] | 6-(4-{2- [(dimethylamino)methyl]-5,6- dimethylpyrimidin-4-yl}- 2,3,4,5-tetrahydro-1,4- benzoxazepin-7- yl)[1,3]thiazolo[5,4-b]pyridin-2- amine |
| 672 | 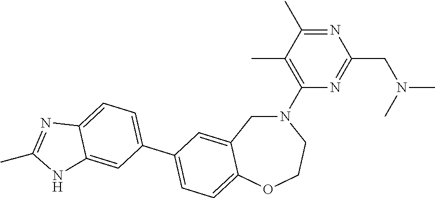 | 1-{4,5-dimethyl-6-[7-(2-methyl- 1H-benzimidazol-6-yl)-2,3- dihydro-1,4-benzoxazepin- 4(5H)-yl]pyrimidin-2-yl}-N,N- dimethylmethanamine |
| 673 | 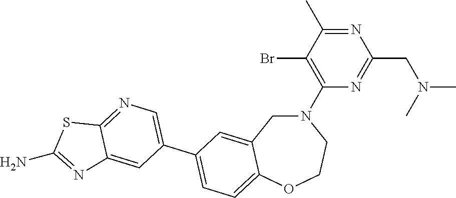 [SMILES: Cc1nc(CN(C)C)nc(N2CCOc3ccc(-c4cnc5sc(N)nc5c4)cc3C2)c1Br] | 6-(4-{5-bromo-2- [(dimethylamino)methyl]-6- methylpyrimidin-4-yl}-2,3,4,5- tetrahydro-1,4-benzoxazepin-7- yl)[1,3]thiazoio[5,4-b]pyridin-2- amine |
| 674 | 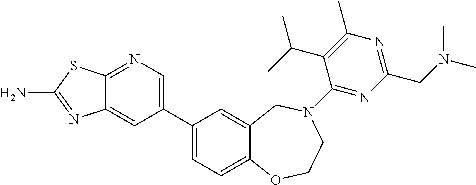 [SMILES: Cc1nc(CN(C)C)nc(N2CCOc3ccc(-c4cnc5sc(N)nc5c4)cc3C2)c1C(C)C] | 6-(4-{2- [(dimethylamino)methyl]-6- methyl-5-(1- methylethyl)pyrimidin-4-yl}- 2,3,4,5-tetrahydro-1,4- benzoxazepin-7- yl)[1,3]thiazolo[5,4-b]pyridin-2- amine |
| 675 | 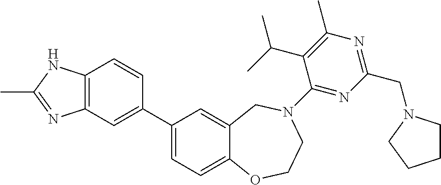 [SMILES: Cc1nc2cc(-c3ccc4c(c3)CN(c3nc(CN5CCCC5)nc(C)c3C(C)C)CCO4)ccc2[nH]1] | 7-(2-methyl-1H-benzimidazol-5- yl)-4-[6-methyl-5-(1- methylethyl)-2-(pyrrolidin-1- ylmethyl)pyrimidin-4-yl]- 2,3,4,5-tetrahydro-1,4- benzoxazepine |
| 676 | 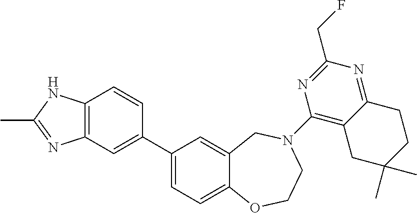 | 4-[2-(fluoromethyl)-6,6- dimethyl-5,6,7,8- tetrahydroquinazolin-4-yl]-7-(2- methyl-1H-benzimidazol-5-yl)- 2,3,4,5-tetrahydro-1,4- benzoxazepine |
| 677 | 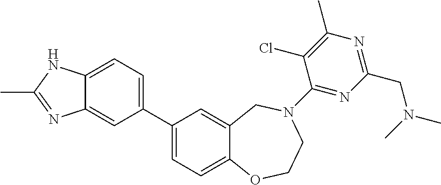 | 1-{5-chloro-4-methyl-6-[7-(2- methyl-1H-benzimidazol-5-yl)- 2,3-dihydro-1,4-benzoxazepin- 4(5H)-yl]pyrimidin-2-yl}-N,N- dimethylmethanamine |
| 678 | 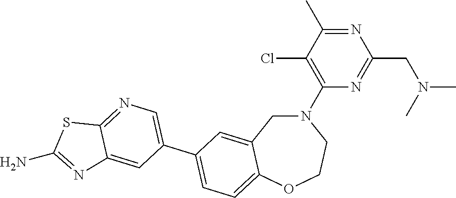 [SMILES: Cc1nc(CN(C)C)nc(N2CCOc3ccc(-c4cnc5sc(N)nc5c4)cc3C2)c1Cl] | 6-(4-{5-chloro-2- [(dimethylamino)methyl]-6- methylpyrimidin-4-yl}-2,3,4,5- tetrahydro-1,4-benzoxazepin-7- yl)[1,3]thiazolo[5,4-b]pyridin-2- amine |
| 679 | 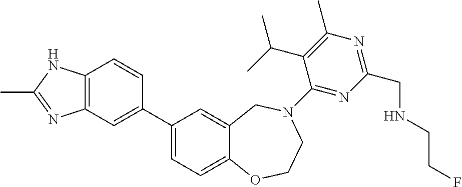 [SMILES: Cc1nc2cc(-c3ccc4c(c3)CN(c3nc(CNCCF)nc(C)c3C(C)C)CCO4)ccc2[nH]1] | 2-fluoro-N-({4-methyl-6-[7-(2- methyl-1H-benzimidazol-5-yl)- 2,3-dihydro-1,4-benzoxazepin- 4(5H)-yl]-5-(1- methylethyl)pyrimidin-2- yl}methyl)ethanamine |
| 680 | 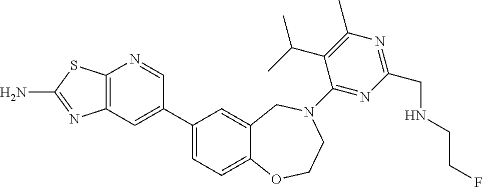 [SMILES: Cc1nc(CNCCF)nc(N2CCOc3ccc(-c4cnc5sc(N)nc5c4)cc3C2)c1C(C)C] | 6-{4-(2-{[(2- fluoroethyl)amino]methyl}-6- methyl-5-(1- methylethyl)pyrimidin-4-yl]- 2,3,4,5-tetrahydro-1,4- benzoxazepin-7- yl}[1,3]thiazolo[5,4-b]pyridin-2- amine |
| 681 | 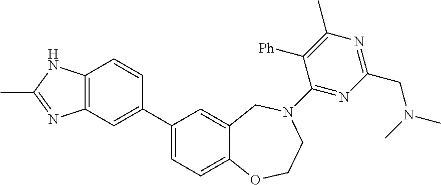 | N,N-dimethyl-1-{4-methyl-6-[7- (2-methyl-1H-benzimidazol-5- yl)-2,3-dihydro-1,4- benzoxazepin-4(5H)-yl]-5- phenylpyrimidin-2- yl}methanamine |
| 682 | 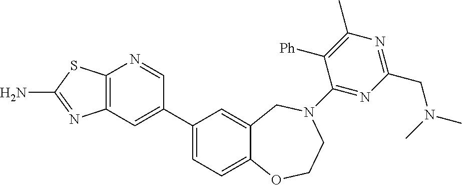 [SMILES: Cc1nc(CN(C)C)nc(N2CCOc3ccc(-c4cnc5sc(N)nc5c4)cc3C2)c1-c1ccccc1] | 6-(4-{2- [(dimethylamino)methyl]-6- methyl-5-phenylpyrimidin-4- yl}-2,3,4,5-tetrahydro-1,4- benzoxazepin-7- yl)[1,3]thiazolo[5,4-b]pyridin-2- amine |
| 683 | 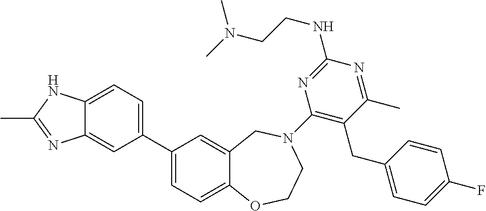 [SMILES: Cc1nc2cc(-c3ccc4c(c3)CN(c3nc(NCCN(C)C)nc(C)c3Cc3ccc(F)cc3)CCO4)ccc2[nH]1] | N′-{5-[(4-fluorophenyl)methyl]- 4-methyl-6-{7-(2-methyl-1H- benzimidazol-5-yl)-2,3-dihydro- 1,4-benzoxazepin-4(5H)- yl]pyrimidin-2-yl}-N,N- dimethylethane-1,2-diamine |
| 684 | 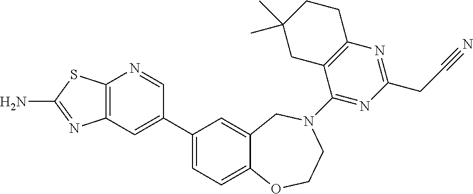 [SMILES: CC1(C)CCc2nc(CC#N)nc(N3CCOc4ccc(-c5cnc6sc(N)nc6c5)cc4C3)c2C1] | {4-[7-(2-amino[1,3]thiazolo[5,4- b]pyridin-6-yl)-2,3-dihydro-1,4- benzoxazepin-4(5H)-yl]-6,6- dimethyl-5,6,7,8- tetrahydroquinazolin-2- yl}acetonitrile |
| 685 | 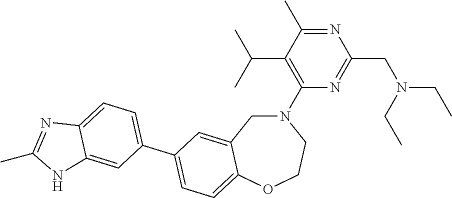 [SMILES: CCN(CC)Cc1nc(C)c(C(C)C)c(N2CCOc3ccc(-c4ccc5nc(C)[nH]c5c4)cc3C2)n1] | N-ethyl-N-({4-methyl-6-[7-(2- methyl-1H-benzimidazol-5-yl)- 2,3-dihydro-1,4-benzoxazepin- 4(5H)-yl]-5-(1- methylethyl)pyrimidin-2- yl}methyl)ethanamine |
| 686 | 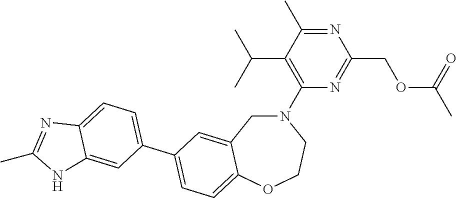 [SMILES: CC(=O)OCc1nc(C)c(C(C)C)c(N2CCOc3ccc(-c4ccc5nc(C)[nH]c5c4)cc3C2)n1] | {4-methyl-6-[7-(2-methyl-1H- benzimidazol-5-yl)-2,3-dihydro- 1,4-benzoxazepin-4(5H)-yl]-5- (1-methylethyl)pyrimidin-2- yl}methyl acetate |
| 687 | 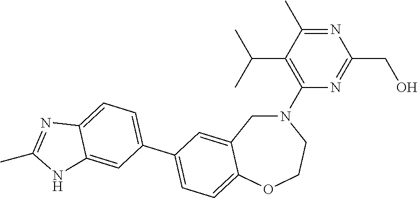 [SMILES: Cc1nc2ccc(-c3ccc4c(c3)CN(c3nc(CO)nc(C)c3C(C)C)CCO4)cc2[nH]1] | {4-methyl-6-[7-(2-methyl-1H- benzimidazol-5-yl)-2,3-dihydro- 1,4-benzoxazepin-4(5H)-yl]-5- (1-methylethyl)pyrimidin-2- yl}methanol |
| 688 | 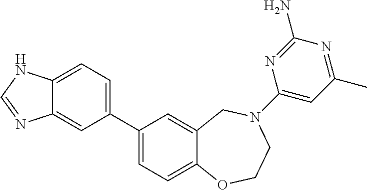 [SMILES: Cc1cc(N2CCOc3ccc(-c4ccc5[nH]cnc5c4)cc3C2)nc(N)n1] | 4-[7-(1H-benzimidazol-5-yl)- 2,3-dihydro-1,4-benzoxazepin- 4(5H)-yl]-6-methylpyrimidin-2- amine |
| 689 | 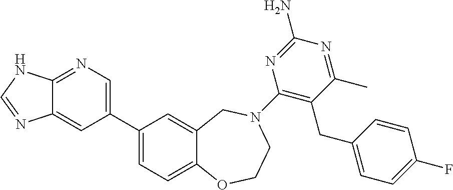 [SMILES: Cc1nc(N)nc(N2CCOc3ccc(-c4cnc5[nH]cnc5c4)cc3C2)c1Cc1ccc(F)cc1] | 5-[(4-fluorophenyl)methyl]-4-[7- (3H-imidazo[4,5-b]pyridin-6- yl)-2,3-dihydro-1,4- benzoxazepin-4(5H)-yl]-6- methylpyrimidin-2-amine |
| 690 | 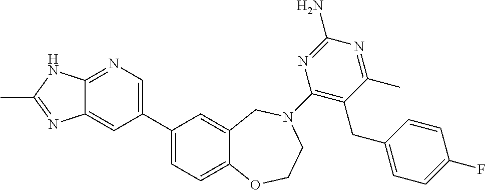 [SMILES: Cc1nc2cc(-c3ccc4c(c3)CN(c3nc(N)nc(C)c3Cc3ccc(F)cc3)CCO4)cnc2[nH]1] | 5-[(4-fluorophenyl)methyl]-4- methyl-6-[7-(2-methyl-3H- imidazo[4,5-b]pyridin-6-yl)-2,3- dihydro-1,4-benzoxazepin- 4(5H)-yl]pyrimidin-2-amine |
| 691 | 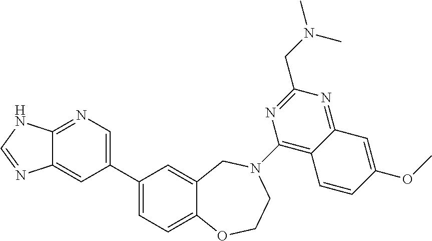 [SMILES: COc1ccc2c(N3CCOc4ccc(-c5cnc6[nH]cnc6c5)cc4C3)nc(CN(C)C)nc2c1] | l-{4-[7-(3H-imidazo[4,5- b]pyddin-6-yl)-2,3-dihydro-1,4- benzoxazepin-4(5H)-yl]-7- (methyloxy)quinazolin-2-yl}- N,N-dimethylmethanamine |
| 692 | 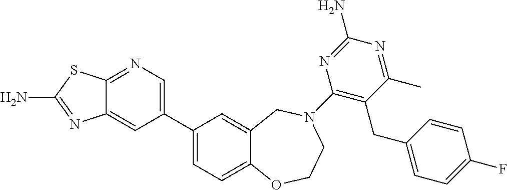 [SMILES: Cc1nc(N)nc(N2CCOc3ccc(-c4cnc5sc(N)nc5c4)cc3C2)c1Cc1ccc(F)cc1] | 6-(4-{2-amino-5-[(4- fluorophenyl)methyl]-6- methylpyrimidin-4-yl}-2,3,4,5- tetrahydro-1,4-benzoxazepin-7- yl)[1,3]thiazolo[5,4-b]pyridin-2- amine |
| 693 | 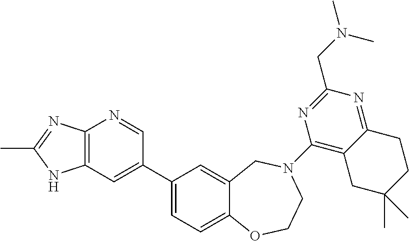 | |
| 694 | 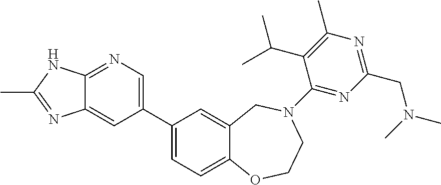 | |
| 695 | 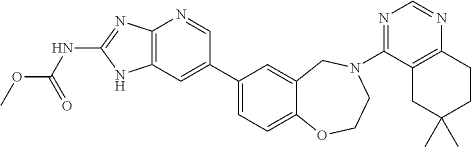 [SMILES: COC(=O)Nc1nc2ncc(-c3ccc4c(c3)CN(c3ncnc5c3CC(C)(C)CC5)CCO4)cc2[nH]1] | |
| 696 | 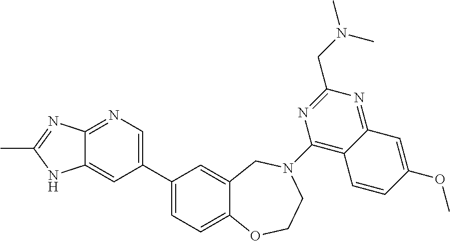 | |
| 697 | 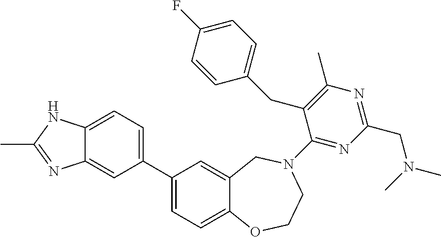 | |
| 698 | 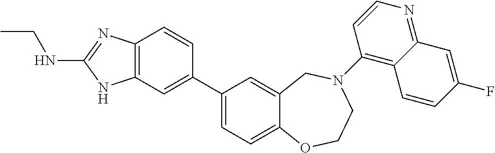 [SMILES: CCNc1nc2ccc(-c3ccc4c(c3)CN(c3ccnc5cc(F)ccc35)CCO4)cc2[nH]1] | |
| 699 | 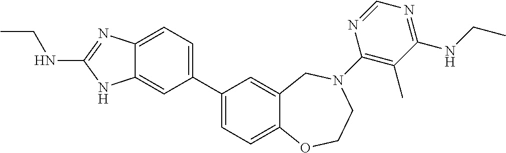 [SMILES: CCNc1nc2ccc(-c3ccc4c(c3)CN(c3ncnc(NCC)c3C)CCO4)cc2[nH]1] | |
| 700 | 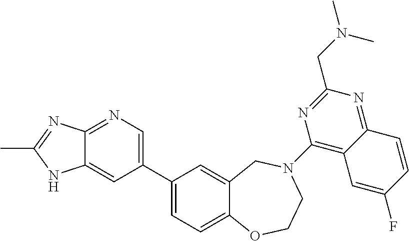 [SMILES: Cc1nc2ncc(-c3ccc4c(c3)CN(c3nc(CN(C)C)nc5ccc(F)cc35)CCO4)cc2[nH]1] | |
| 701 | 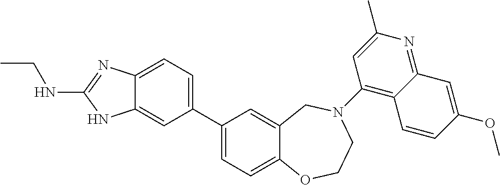 [SMILES: CCNc1nc2ccc(-c3ccc4c(c3)CN(c3cc(C)nc5cc(OC)ccc35)CCO4)cc2[nH]1] | |
| 702 | 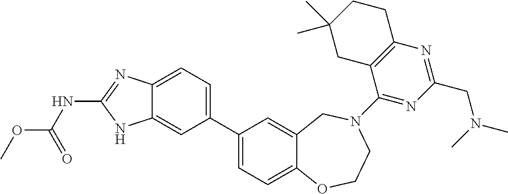 | |
| 703 | 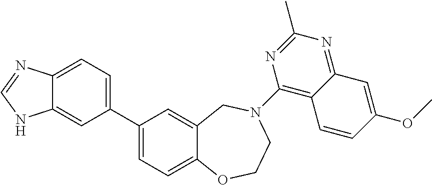 [SMILES: COc1ccc2c(N3CCOc4ccc(-c5ccc6nc[nH]c6c5)cc4C3)nc(C)nc2c1] | |
| 704 | 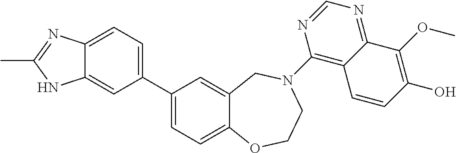 | |
| 705 | 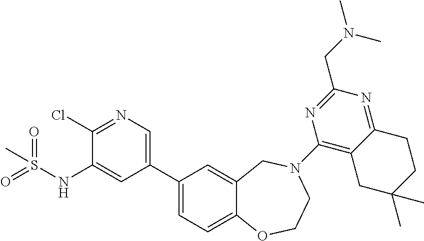 [SMILES: CN(C)Cc1nc2c(c(N3CCOc4ccc(-c5cnc(Cl)c(NS(C)(=O)=O)c5)cc4C3)n1)CC(C)(C)CC2] | |

Useful Intermediates: 4-[6,7-bis(methyloxy)quinolin-4-yl]-7-bromo-2,3,4,5-tetrahydro-1,4-benzoxazepine; 4-{4-[6,7-bis(methyloxy)quinolin-4-yl]-2,3,4,5-tetrahydro-1,4-benzoxazepin-7-yl}-2-nitroaniline; 4-{4-[6,7-bis(methyloxy)quinolin-4-yl]-2,3,4,5-tetrahydro-1,4-benzoxazepin-7-yl}benzene-1,2-diamine; N-[5-(4-{5-[(4-fluorophenyl)methyl]-6-methylpyrimidine-4-yl}-2,3,4,5-tetrahydro-1,4-benzoxazepin-7-yl)-1,3-thiazol-2-yl]acetamide; 7-bromo-4-{5-[(4-fluorophenyl)methyl]-6-methylpyrimidin-4-yl}-2,3,4,5-tetrahydro-1,4-benzoxazepine; 4-[6,7-bis(methyloxy)quinazolin-4-yl]-7-bromo-2,3,4,5-tetrahydro-1,4-benzoxazepine; 7-bromo-4-[6-(methyloxy)quinazolin-4-yl]-2,3,4,5-tetrahydro-1,4-benzoxazepine.

### General Administration

In one aspect, the invention provides pharmaceutical compositions comprising an inhibitor of PI3K and/or mTOR according to the invention and a pharmaceutically acceptable carrier, excipient, or diluent. In certain other specific embodiments, administration is by the oral route. Administration of the compounds of the invention, or their pharmaceutically acceptable salts, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as for example, tablets, suppositories, pills, soft elastic and hard gelatin capsules, powders, solutions, suspensions, or aerosols, or the like, specifically in unit dosage forms suitable for simple administration of precise dosages.

The compositions will include a conventional pharmaceutical carrier or excipient and a Compound of the invention as the/an active agent, and, in addition, may include carriers and adjuvants, etc.

Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylalted hydroxytoluene, etc.

The choice of formulation depends on various factors such as the mode of drug administration (e.g., for oral administration, formulations in the form of tablets, pills or capsules) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

One specific route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the active Compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents, and can also be of such composition that they release the active Compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., a compound(s) of the invention, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds of the present invention with for example suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration of a Compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

Compressed gases may be used to disperse a Compound of this invention in aerosol form. Inert gases suitable for this purpose are nitrogen, carbon dioxide, etc.

Generally, depending on the intended mode of administration, the pharmaceutically acceptable compositions will contain about 1% to about 99% by weight of a compound(s) of the invention, or a pharmaceutically acceptable salt thereof, and 99% to 1% by weight of a suitable pharmaceutical excipient. In one example, the composition will be between about 5% and about 75% by weight of a compound(s) of the invention, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a Compound of the invention, or a pharmaceutically acceptable salt thereof, for treatment of a disease-state in accordance with the teachings of this invention.

The compounds of the invention, or their pharmaceutically acceptable salts or solvates, are administered in a therapeutically effective amount which will vary depending upon a variety of factors including the activity of the specific Compound employed, the metabolic stability and length of action of the compound, the age, body weight, general health, sex, diet, mode and time of administration, rate of excretion, drug combination, the severity of the particular disease-states, and the host undergoing therapy. The compounds of the present invention can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the Compound being used. The determination of optimum dosages for a particular patient is well known to one of ordinary skill in the art.

If formulated as a fixed dose, such combination products employ the compounds of this invention within the dosage range described above and the other pharmaceutically active agent(s) within its approved dosage range. Compounds of the instant invention may alternatively be used sequentially with known pharmaceutically acceptable agent(s) when a combination formulation is inappropriate.

### General Synthesis

Compounds of this invention can be made by the synthetic procedures described below. The starting materials and reagents used in preparing these compounds are either available from commercial suppliers such as Aldrich Chemical Co. (Milwaukee, Wis.), or Bachem (Torrance, Calif.), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4^{th} Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure. The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure and over a temperature range from about −78° C. to about 150° C., more specifically from about 0° C. to about 125° C. and more specifically at about room (or ambient) temperature, e.g., about 20° C. Unless otherwise stated (as in the case of an hydrogenation), all reactions are performed under an atmosphere of nitrogen.

Prodrugs can be prepared by techniques known to one skilled in the art. These techniques generally modify appropriate functional groups in a given compound. These modified functional groups regenerate original functional groups by routine manipulation or in vivo. Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

The compounds of the invention, or their pharmaceutically acceptable salts, may have asymmetric carbon atoms or quaternized nitrogen atoms in their structure. Compounds of the Invention may exist as single stereoisomers, racemates, and as mixtures of enantiomers and diastereomers. The compounds may also exist as geometric isomers. All such single stereoisomers, racemates and mixtures thereof, and geometric isomers are intended to be within the scope of this invention.

Some of the compounds of the invention contain an active ketone —C(O)CF₃ and may exist in part or in whole as the —C(OH₂)CF₃ form. Regardless of whether the Compound is drawn as the —C(O)CF₃ or —C(OH₂)CF₃ form, both are included within the scope of the Invention. Although an individual Compound may be drawn as the —C(O)CF₃ form, one of ordinary skill in the art would understand that the Compound may exist in part or in whole as the —C(OH₂)CF₃ form and that the ratio of the two forms may vary depending on the Compound and the conditions in which it exists.

Some of the compounds of the invention may exist as tautomers. For example, where a ketone or aldehyde is present, the molecule may exist in the enol form; where an amide is present, the molecule may exist as the imidic acid; and where an enamine is present, the molecule may exist as an imine. All such tautomers are within the scope of the invention. Further, for example, in this application R¹ can be 5-oxo-1H-1,2,4-triazol-3-yl, depicted structurally as

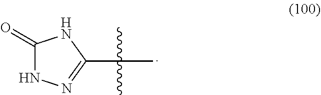 [SMILES: *c1n[nH]c(=O)[nH]1]

Both 5-oxo-1H-1,2,4-triazol-3-yl and the structure 100 include, and are equivalent to, 3-hydroxy-4H-1,2,4-triazol-5-yl and its structure

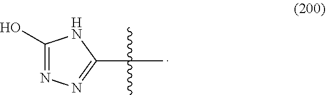 [SMILES: *c1nnc(O)[nH]1]

In another example, in this application R¹ can be 2-imino-1(2H)-hydroxy-pyrimidin-5-yl, depicted structurally as

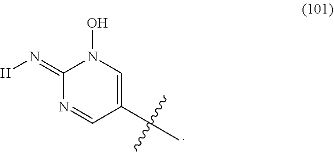 [SMILES: *c1cn/c(=N\[H])n(O)c1]

Both 2-imino-1(2H)-hydroxy-pyrimidin-5-yl and the structure 101 include, and are equivalent to, N-oxide of 2-amino-pyrimidin-5-yl and its structure 201:

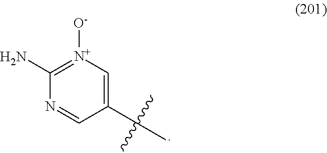 [SMILES: *c1cnc(N)[n+]([O-])c1]

Regardless of which structure or which terminology is used, each tautomer is included within the scope of the Invention.

The present invention also includes N-oxide derivatives and protected derivatives of compounds of the Invention. For example, when compounds of the Invention contain an oxidizable nitrogen atom, the nitrogen atom can be converted to an N-oxide by methods well known in the art. When compounds of the Invention contain groups such as hydroxy, carboxy, thiol or any group containing a nitrogen atom(s), these groups can be protected with a suitable “protecting group” or “protective group”. A comprehensive list of suitable protective groups can be found in T. W. Greene, Protective Groups in Organic Synthesis, John Wiley & Sons, Inc. 1991, the disclosure of which is incorporated herein by reference in its entirety. The protected derivatives of compounds of the Invention can be prepared by methods well known in the art.

Methods for the preparation and/or separation and isolation of single stereoisomers from racemic mixtures or non-racemic mixtures of stereoisomers are well known in the art. For example, optically active (R)- and (S) isomers may be prepared using chiral synthons or chiral reagents, or resolved using conventional techniques. Enantiomers (R- and S-isomers) may be resolved by methods known to one of ordinary skill in the art, for example by: formation of diastereoisomeric salts or complexes which may be separated, for example, by crystallization; via formation of diastereoisomeric derivatives which may be separated, for example, by crystallization, selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts or solvents or by converting on enantiomer to the other by asymmetric transformation. For a mixture of enantiomers, enriched in a particular enantiomer, the major component enantiomer may be further enriched (with concomitant loss in yield) by recrystallization.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The chemistry for the preparation of the compounds of this invention is known to those skilled in the art. In fact, there may be more than one process to prepare the compounds of the invention. The following examples illustrate but do not limit the invention. All references cited herein are incorporated by reference in their entirety.

An intermediate of formula 4 where PG is a nitrogen-protecting group, R^{5a} and R^{5c} are independently hydrogen or alkyl, R^{5h} is hydrogen or halo, R^{5b} is hydrogen, amino, or halo, and R^{5d}, R^{5e}, R^{5f}, and R^{5g} are hydrogen can be prepared according to Scheme 1.

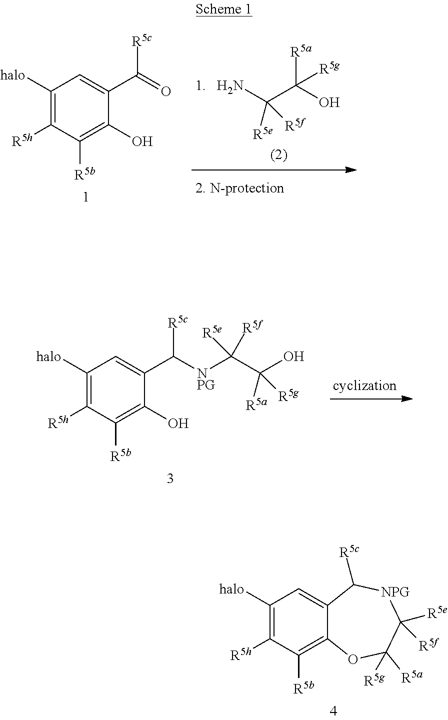

In particular, an intermediate of formula 4a can be prepared according to Scheme 1a.

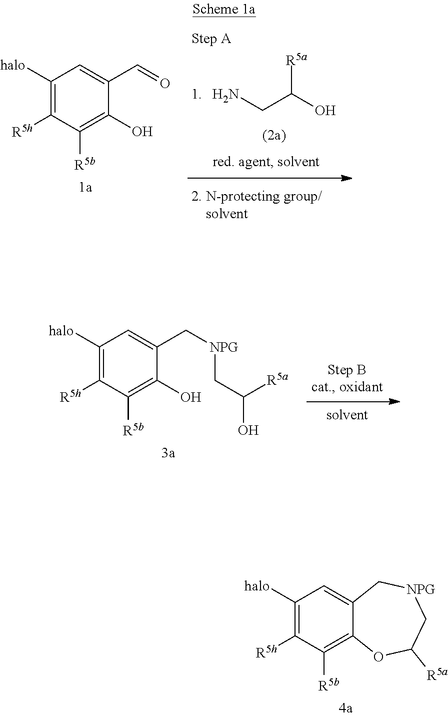

An intermediate of Formula 1a is commercially available or can be prepared using methods known to one of ordinary skill in the art. In particular an intermediate of formula 1a where R^{5b} is hydrogen and R^{5h} is hydrogen, bromo, or chloro is commercially available. An intermediate of formula 1a where R^{5h} is hydrogen and R^{5b} is bromo, chloro, iodo, or fluoro is commercially available. An intermediate of formula 1a where R^{5h} is fluoro and R^{5b} is hydrogen can be prepared using procedures described in J. of Med. Chem., 2004, 47(12), 3163-3179. An intermediate of formula 1a where R^{5h} is hydrogen and R^{5b} is amino can be prepared from the corresponding, commercially-available nitro intermediate using procedures known to one of ordinary skill in the art.

An intermediate of formula 2a where R^{5a} is hydrogen or methyl is commercially available. The intermediate of formula 1a is treated with an intermediate of formula 2a in the presence of a reducing agent such as sodium borohydride, in a solvent(s) such as tetrahydrofuran and/or methanol and allowed to react at a temperature of about 40° C. for approximately 4 hours. The solvent is then removed and the reaction is taken up in a solvent(s) such as ethyl acetate and/or saturated sodium bicarbonate. To this suspension a nitrogen-protecting group precursor, such as di-tert-butyl dicarbonate, is added and the mixture is allowed to stir at room temperature overnight to yield an intermediate of formula 3a where PG is a nitrogen-protecting group.

Intermediate 3a is then treated with a catalyst, such as triphenylphosphine, in the presence of a dehydrating agent such as diisopropyl azodicarboxylate, in a solvent such as DCM. The reaction is allowed to proceed at room temperature for approximately 12 hours and the resulting product is optionally purified by column chromatography to yield an intermediate of formula 4a. Alternatively, the intermediate of formula 4a can be prepared by treating the intermediate of formula 3a with Burgess' reagent.

An intermediate of formula 5 where each R is hydrogen or both R's when taken together form a cyclic boronic ester, PG is a nitrogen-protecting group, R^{5a} and R^{5c} are independently hydrogen or alkyl, R^{5h} is hydrogen or halo, R^{5b} is hydrogen, amino, or halo, R^{5e}, R^{5f}, and R^{5g} are hydrogen, and R¹ is as defined in the Summary of the Invention for a Compound of Formula I can be prepared according to Scheme 2.

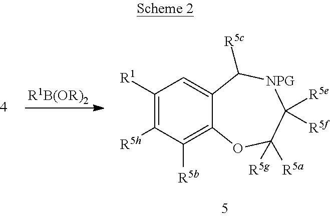

where the intermediate of formula 4 is prepared as described in Scheme 1.

In particular, an intermediate of formula 5a where R^{5a} is hydrogen or alkyl, R^{5h} is hydrogen or halo, R^{5b} is hydrogen, amino, or halo, and R¹ is as defined in the Summary of the Invention for a Compound of Formula I, can be prepared according to Scheme 2a.

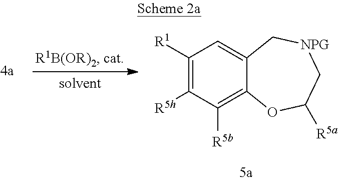

The intermediate of formula 4a, prepared as described in Scheme 1a, is treated with a boronic acid of formula R¹B(OH)₂ or

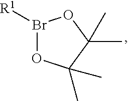

which are commercially available or can be prepared using procedures known to one of ordinary skill in the art. The reaction is carried out in the presence of a catalyst such as Pd(dppf)₂Cl₂, a base such as potassium carbonate, and in a solvent such as DME at about 80° C. for about 2 hours. The product can then be purified by chromatography to yield an intermediate of formula 5a.

Alternatively, an intermediate of formula 5, as defined above, can be prepared as described in Scheme 4.

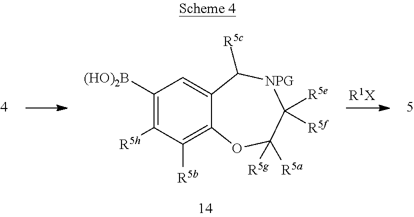

In particular, an intermediate of formula 5b where PG is a nitrogen-protecting group and R¹ is as defined in the Summary of the Invention for a Compound of Formula I can be prepared according to Scheme 4a.

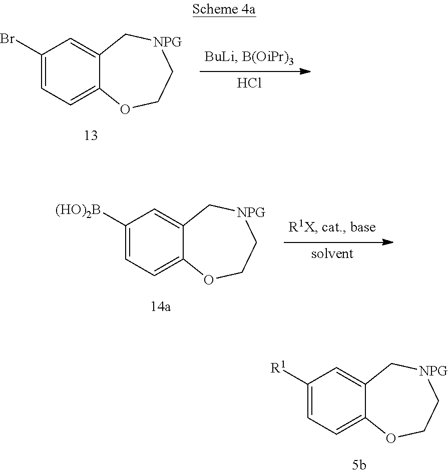

An intermediate of formula 13, where PG is a nitrogen-protecting group, is prepared as described in Scheme 1a. 13 is treated with triisopropylborate in a solvent such as THF at a temperature of about −60° C., followed by dropwise addition of a base such as n-butyllithium in tetrahydrofuran. The reaction was allowed to proceed for about 30 minutes, was treated with an acid such as hydrochloric acid, and allowed to warm to room temperature to yield an intermediate of formula 14a. Intermediate 14a is then treated with an intermediate of formula R¹X (where X is a halide, and which is commercially available or can be prepared using procedures known to one of ordinary skill in the art), in the presence of a base such as potassium carbonate, in the presence of a catalyst such as tetrakis(triphenylphosphine)palladium(0), and in a solvent(s) such as 1,2-dimethoxyethane and/or water. The reaction is allowed to proceed under nitrogen and stirred at reflux for about 3 hours to yield an intermediate of formula 5b.

In particular, a Compound of the Invention where Y is ═CH— or ═N—, R^{5a}, R^{5b}, R^{5c}, R^{5d}, R^{5e}, R^{5f}, R^{5g}, and R^{5h} are hydrogen; R¹ is benzimidazol-6-yl substituted at the 2-position with one R⁷; R⁷ is alkyl; R² and all other groups are independently as defined in the Summary of the Invention for a Compound of Formula I, can be prepared according to Scheme 6a.

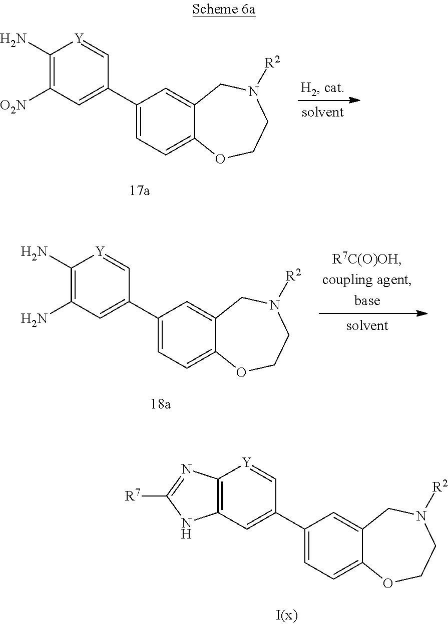

The nitro of the intermediate of formula 17a, prepared as described above in Scheme 4, is reduced in the presence of H₂ and palladium on carbon in a solvent(s) such as methanol and/or acetic acid to yield an intermediate of formula 18a. The intermediate of formula 18a is then treated with an intermediate of formula R⁷C(O)OH, in the presence of a coupling agent such as HATU, in the presence of a base such as DIEA, in a solvent(s) such as DMF and/or acetic acid. The product can be purified by column chromatography to yield a Compound of Formula I(x).

A Compound of the Invention of Formula I where R^{5a} and R^{5c} are independently hydrogen or alkyl, R^{5h} is hydrogen or halo, R^{5b} is hydrogen, amino, or halo, R^{5e}, R^{5f}, and R^{5g} are hydrogen, and R¹ and R² are independently as defined in the Summary of the Invention for a Compound of Formula I can be prepared as described in Scheme 5,

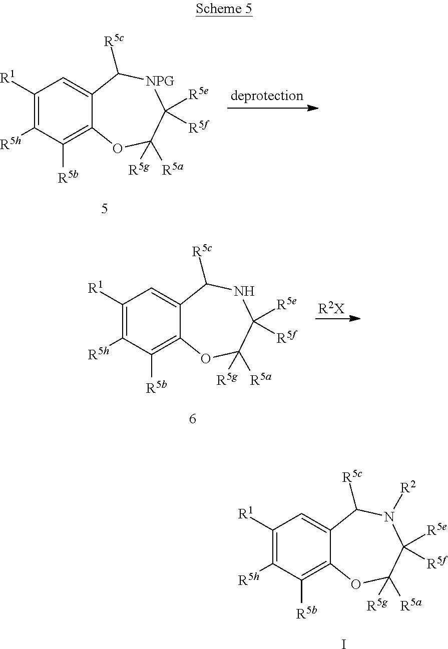

where X is halo or hydroxy.

In particular, a Compound of Formula I(w) where R^{5a} is hydrogen or alkyl, R^{5h} is hydrogen or halo, R^{5b} is hydrogen, amino, or halo, and R¹ and R² are independently as defined in the Summary of the Invention for a Compound of Formula I can be prepared as described in Scheme 5a.

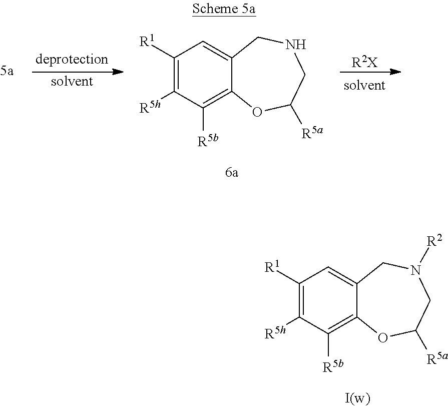

The protecting group on the intermediate of formula 5a is removed. When the protecting group is Boc, it can be removed with HCl to yield an intermediate of formula 6a. The intermediate of formula R²X (where X is a leaving group such as halo) is commercially available or can be prepared using procedures described herein or procedures known to one of ordinary skill in the art. The intermediate of formula 6a is then treated with R²X, in the presence of a base such as Hünig's base or NMP, in a solvent such as DMF, at a temperature of about 50° C. The product can be purified by column chromatography to yield an intermediate of Formula I(w).

In particular, a Compound of Formula I(a) where R¹ and R² are independently as defined in the Summary of the Invention for a Compound of Formula I can be prepared according to Scheme 5b.

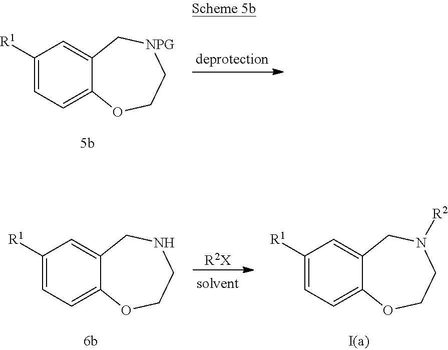

The protecting group on intermediate of formula 5b, prepared as described in Scheme 4a, is removed. When the protecting group is Boc, it can be removed with HCl to yield an intermediate of formula 6b. Intermediate 6b is then treated with an intermediate of formula R²X where X is a leaving group such as halo using standard alkylating conditions to yield a Compound of Formula I(a).

A Compound of Formual I(aa) where one of Y₁ and Y₂ is ═CH— and the other is ═N—, R¹ is benzimidazol-6-yl substituted at the 2-position with one R⁷; R⁷ and R² are independently as defined in the Summary of the Invention for a Compound of Formula I can be prepared according to Scheme 6a using conditions known to one of ordinary skill in the art.

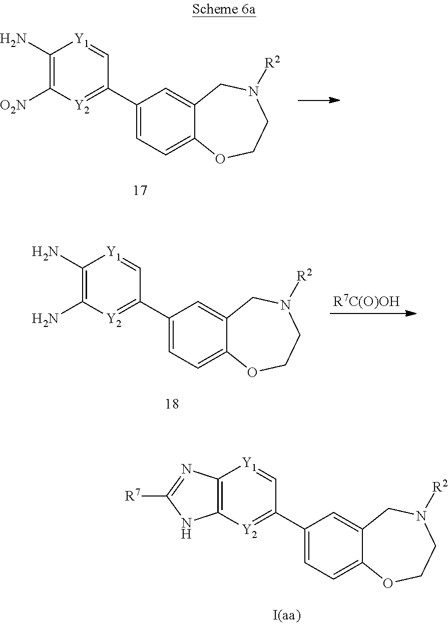

An intermediate of formula 17 is prepared by 1) treating an intermediate of formula 14a, prepared as described in Scheme 4a, with an intermediate of formula

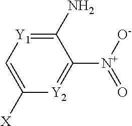

where X is halo using standard Suzuki coupling conditions; followed by 2) treating the with and intermediate of formula R²X using standard alkylating conditions. 17 is then hydrogenated in the presence of palladium on carbon in a solvent such as acetic acid to yield the intermediate of formula 18. 18 is then treated with an acid of formula R⁷C(O)OH to yield the Compound of Formula I(aa).

Alternatively, a Compound of Formula I(aa) can be prepared according to Scheme 6b.

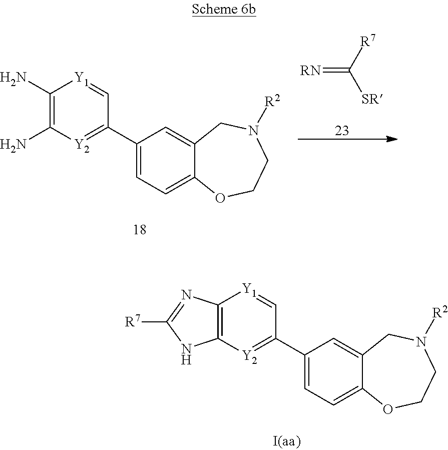

The intermediate of formula 18 is treated with an intermediate of formula 23 in the presence of glacial acetic acid, optionally in the presence of triethyl orthoformate, and heated to yield an a Compound of Formula I(aa).

A Compound of Formula I(v) where R² is as defined in the Summary of the Invention for a Compound of Formula I can be prepared according to Scheme 7a.

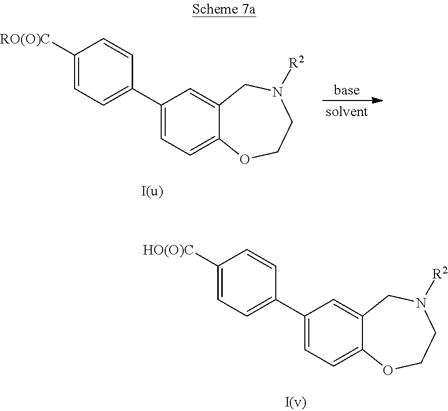

The Compound of Formula I(u) where R is alkyl, prepared using procedures according to Scheme 5b, is treated with a base such as LiOH, in a solvent(s) such as THF and/or water to yield the hydrolyzed Compound of Formula I(y).

A Compound of Formula I(z) where R², R⁸, and R^{8a} are independently as defined in the Summary of the Invention for a Compound of Formula I can be prepared according to Scheme 7b.

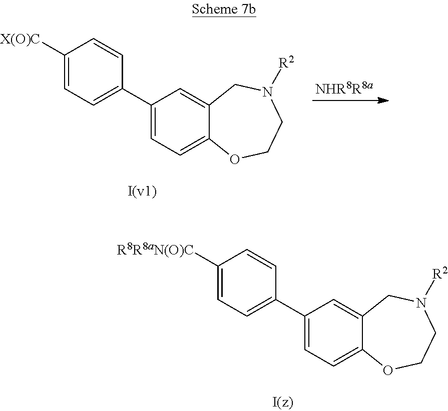

The Compound of Formula I(v1) where X is halo or hydroxy can be prepared according to Scheme 7a or prepared by making the acid chloride from a Compound of Formula I(v). The Compound of Formula I(v1) is then treated with an amine of formula NHR⁸R^{8a} optionally in the presence of a base such as DIEA in a solvent such as THF to yield a Compound of Formula I(z).

A Compound of Formula I where R¹, R², R^{5a}, R^{5b}, R^{5c}, R^{5d}, R^{5e}, R^{5f}, R^{5g}, R^{5g}, and R^{5h} can be prepared according to the following scheme (where R is —B(OH)₂ and Y is halo, or R is halo and Y is —B(OH)₂) using Suzuki coupling procedures known to one of ordinary skill in the art.

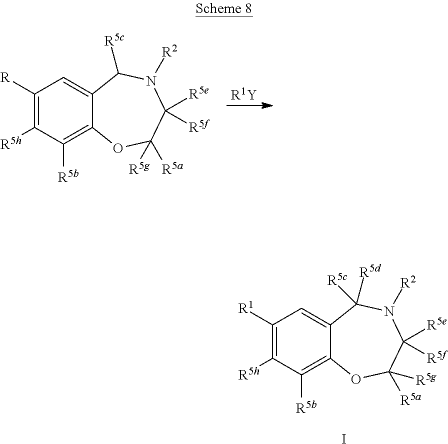

In particular, a Compound of Formula I(a) where R¹ and R² are independently as defined in the Summary of the Invention for a Compound of Formula I can be prepared as described in Scheme 8a.

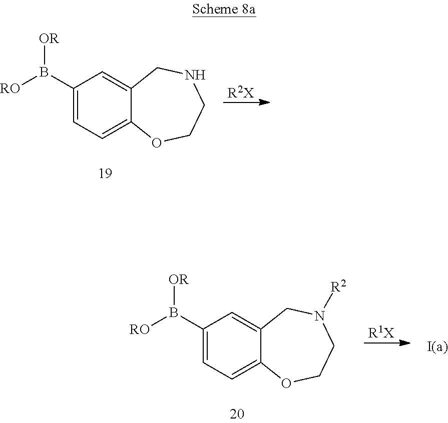

An intermediate of formula 19 (where each R is hydrogen or the two R's together form a boronic ester), which can be prepared by following step 1 of Scheme 4a and subsequent deprotection, is treated with an intermediate of formula R²X in a solvent such as dioxane/H₂O and in the presence of a base such as DIPEA. The resulting mixture is heated to about 90° C. to yield an intermediate of formula 20. 20 is treated with an intermediate of formul R¹X where X is halo and R¹ is as defined in the Summary of the Invention for a Compound of Formula I in a solvent such as DMF/water, in the presence of a base such as DIEA, in the presence of a catalyst such as [1,1′-bis(diphenylphosphino)ferrocene]dichloropalladium(II). The reaction is heated to about 95° C. 20 is then optionally purified to yield a Compound of Formula I(a).

Alternatively, a Compound of Formula I(a) where R¹ and R² are independently as defined in the Summary of the Invention for a Compound of Formula I can be prepared as described in Scheme 8b.

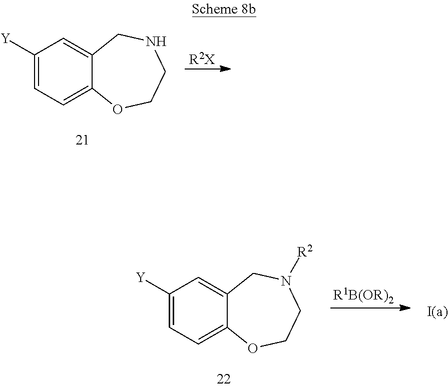

An intermediate of formula 21 where Y is halo, which can be prepared by following Scheme 1a followed by deprotection, is treated with an intermediate of formula R²X where X is halo, a base such as DIEA in a solvent such as 1-butanol and heated to yield an intermediate of formula 22. 22 is then treated with an intermediate of formula R¹B(OR)₂ (where each R is hydrogen or the two R together form a boronic ester), in the presence of a base such as potassium carbonate and in the presence of a catalyst such as dichloro[1,1-bis(diphenylphosphino]ferrocenepalladium (II) dichloromethane adduct in a solvent such as dimethoxyethane/water. The reaction was heated and yielded a Compound of Formula I(a).

### Synthetic Examples

### Reagent Preparation 1

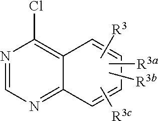

STEP 1: A solution of methyl 2-amino-5-bromo-4-methoxybenzoate (75 mg, 0.29 mmol) and ammonium formate (38 mg, 0.8 mmol) in formamide (1 mL) was heated at 165° C. for 18 h. The mixture was allowed to cool to room temperature then diluted with an excess of water. The solid formed was collected by filtration and washed with water then ethyl acetate and dried to give 6-bromo-7-methoxyquinazolin-4(3H)-one (53 mg, 72% yield) as a pale yellow solid. MS (EI) for C₉H₇BrN₂O₂: 255, 257 (MH⁺).

STEP 2: 6-bromo-7-methoxyquinazolin-4(3H)-one (53 mg, 0.21 mmol) was taken into thionyl chloride (1.5 mL) followed by addition of catalytic DMF. The mixture was heated to 80° C. for 2 h then concentrated. The residue was partitioned with ethyl acetate and saturated aqueous sodium bicarbonate. The organic phase was washed with brine then dried over anhydrous sodium sulfate, filtered and concentrated to give 6-bromo-4-chloro-7-methoxyquinazoline (36 mg, 62% yield) as a brown solid. MS (EI) for C₉H₆BrClN₂O: 275 (MH⁺).

Using analogous synthetic techniques and substituting with alternative starting reagents in step 1 the following reagents were prepared.

4-chloro-7-(methylsulfonyl)quinazoline. Synthesized according to the method of reagent preparation 1 using 7-(methylsulfonyl)quinazolin-4(3H)-one in step 2. ¹H NMR (400 MHz, d₆-DMSO): 8.36 (d, 1H), 8.34 (s, 1H), 8.18 (d, 1H), 8.02 (dd, 1H), 3.36 (s, 3H).

4,7-dichloro-6-iodoquinazoline. Synthesized according to the method of reagent preparation 1 using methyl 2-amino-4-chloro-5-iodobenzoate in step 1. MS (EI) for C₈H₃Cl₂₁N₂: 325 (MH⁺).

4-chloro-6-iodo-8-methylquinazoline. Synthesized according to the method of reagent preparation 1 using 2-amino-5-iodo-3-methylbenzoic acid in step 1. MS (EI) for C₉H₆Cl₁N₂: 305 (MH⁺).

4-chloro-6-(phenylmethoxy)-quinazoline. Prepared according to the method of reagent preparation 1 using 2-amino-5-benzyloxybenzoic acid methyl ester (J. Org. Chem. 2001, 66(8), 2784-2788) in step 1. MS (EI) for C₁₅H₁₁ClN₂O: 271 (MH⁺).

4,6-dichloro-7-methoxy-quinazoline. Prepared according to the method of reagent preparation 1 using 5-chloro-4-methoxyanthranilic acid (US 80-126838) in step 1. MS (EI) for C₉H₆Cl₂N₂O: 271 (MH⁺).

4-chloro-7,8-dimethoxy-quinazoline. Prepared according to the method of reagent preparation 1 using 2-amino-3,4-dimethoxybenzoic acid methyl ester (U.S. Pat. No. 4,287,341) in step 1. MS (EI) for C₁₀H₉ClN₂O₂: 225 MH⁺).

7-(benzyloxy)-4-chloro-8-methoxyquinazoline. Prepared according to the method of reagent preparation 1 using 2-amino-3-methoxy-4-(phenylmethoxy)benzoic acid (J. Med. Chem. 1992, 35(14), 2703-10) in step 1. MS (EI) for C₁₆H₁₃ClN₂O₂: 301 MH⁺).

4,6-dichloro-7,8-dimethoxyquinazoline. Prepared according to the method of reagent preparation 1 using 2-amino-5-chloro-3,4-dimethoxybenzoic acid (U.S. Pat. No. 4,287,341) in step 1. MS (EI) for C₁₀H₈Cl₂N₂O₂: 260 MH⁺).

6-bromo-4,7-dichloroquinazoline. Synthesized according to the method of reagent preparation 1 by using 2-amino-5-bromo-4-chlorobenzoic acid in step 1. MS (EI) for C₈H₃BrCl₂N₂: 277 (MH⁺).

4-chloro-6-iodo-7-methoxyquinazoline. Synthesized according to the method of reagent preparation 1 by N-iodosuccinimide iodination of methyl 2-amino-4-methoxybenzoate to give methyl 5-iodo-2-amino-4-methoxybenzoate then proceeding with step 1. ¹H NMR (400 MHz, CDCl₃): 8.97, (s, 1H), 8.75, 7.31 (s, 1H), 4.08 (s, 3H). GC-MS for C₉H₆ClIN₂O: 319 (M⁺).

7-bromo-4-chloro-8-methoxyquinazoline and 7-bromo-4-chloro-6-methoxyquinazoline. Synthesized according to the method of reagent preparation 1 by nitration and hydrogenation of methyl 4-bromo-3-methoxybenzoate to give a separable mixture of methyl 4-bromo-3-methoxy-2-aminobenzoate and methyl 4-bromo-5-methoxy-2-aminobenzoate then proceeding with step 1 individually. 7-bromo-4-chloro-8-methoxyquinazoline: ¹H NMR (400 MHz, CDCl₃): 9.09, (s, 1H), 7.92 (d, 1H), 7.87 (d, 1H), 4.21 (s, 3H). GC-MS for C₉H₆BrClN₂O: 272 (M⁺). 7-bromo-4-chloro-6-methoxyquinazoline: ¹H NMR (400 MHz, CDCl₃): 8.95, (s, 1H), 8.40 (d, 1H), 7.45 (d, 1H), 4.18 (s, 3H), GC-MS for C₉H₆BrClN₂O: 272 (M⁺).

8-bromo-4-chloro-6-methyl-quinazoline. Synthesized according to the method of reagent preparation 1 using 2-amino-3-bromo-5-methylbenzoic acid in step 1. GC-MS (EI) for C₉H₆BrClN₂: 257 (M⁺).

4-chloro-6-(methylsulfonyl)quinazoline. Synthesized according to the method of reagent preparation 1 using 6-(methylsulfonyl)quinazolin-4(3H)-one in step 2. 6-(methylsulfonyl)quinazolin-4(3H)-one was obtained by the one step oxidation of 6-(methylthio)quinazolin-4(3H)-one (J. Med. Chem. 1983, 26(3), 420-5). MS (EI) for C₉H₇ClN₂O₂: 242 (M⁺).

### Reagent Preparation 2

### 4-chloro-5-methyl-6-(phenylmethyl)pyrimidine

Prepared from 4,6-dichloro-5-methylpyrimidine and benzyl zinc bromide (0.5 M solution in tetrahydrofuran) according to the procedure described in WO 2007/146824 as a colorless oil. ¹H NMR (400 MHz, CDCl₃): 8.78 (s, 1H), 7.33-7.18 (m, 5H), 4.19 (s, 2H), 2.36 (s, 3H); MS (EI) for C₁₂H₁₁ClN₂: 219 (MH⁺).

### Reagent Preparation 3

### 4-chloro-6,6-dimethyl-5,6,7,8-tetrahydroquinazoline

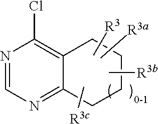

STEP 1: To a cooled (0° C.) solution of 4,4-dimethylcyclohexanone (21 g, 0.17 mol) and dimethyl carbonate (45 g, 0.50 mol) in THF (400 mL) was added NaH (60% wt/wt in mineral oil, 17 g, 0.43 mol) portionwise over 30 minutes. The resulting slurry was allowed to stir at ambient temperature for 30 minutes followed by two hours at reflux. The reaction mixture was cooled (0° C.) and MeOH (30 mL) was added dropwise over 20 minutes. The resulting slurry was partitioned between 10% aqueous citric acid and ethyl acetate. The organic layer was washed with brine, dried over magnesium sulfate and concentrated in vacuo. Purification by vacuum distillation provided methyl 2-hydroxy-5,5-dimethylcyclohex-1-enecarboxylate (22.5 g, 75% yield). ¹H NMR (400 MHz, CDCl₃) δ 12.15 (s, 1H), 3.75 (s, 3H), 2.29 (t, 2H), 2.03 (s, 2H), 1.44 (t, 2H), 0.96 (s, 6H); MS (EI) for C₁₀H₁₆O₃: 184 (M⁺).

STEP 2: A solution of methyl 2-hydroxy-5,5-dimethylcyclohex-1-enecarboxylate (10.0 g, 54 mmol) and ammonium acetate (10 g, 130 mmol) in ethanol (50 mL) was heated to reflux for 2 hours. The reaction was concentrated to one third original volume, and then diluted with ethyl acetate (100 mL). The organic solution was washed with water (100 mL) and brine (50 mL) and then dried over anhydrous sodium sulfate. After filtration and concentration, the residue was purified by silica gel column chromatography (ethyl acetate/hexanes, 1:8) to afford methyl 2-amino-5,5-dimethylcyclohex-1-enecarboxylate (7.42 g, 75% yield) as a yellow solid. MS (EI) for C₁₀H₁₇NO₂: 184 (MH⁺).

STEP 3: 2-amino-5,5-dimethylcyclohex-1-enecarboxylate (7.42 g, 40 mmol) was dissolved in N,N-dimethylformamide dimethylacetal (50 mL) and heated to 110° C. for 18 hours. The resulting solution was cooled to room temperature and concentrated to provide methyl 2-((dimethylamino)methyleneamino)-5,5-dimethylcyclohex-1-enecarboxylate (9.5 g, 98% yield) as an oil. ¹H NMR (400 MHz, CDCl₃): 3.65 (s, 3H), 3.49 (s, 1H), 2.95 (s, 6H), 2.35 (m, 2H), 2.15 (br s, 2H), 1.41 (t, 2H), 0.95 (s, 6H); MS (EI) for C₁₃H₂₂N₂O₂: 239 (MH⁺).

STEP 4: A solution of methyl 2-((dimethylamino)methyleneamino)-5,5-dimethylcyclohex-1-enecarboxylate (9.5 g, 40 mol) in 7.0M ammonia in methanol (35 mL) was stirred at 25° C. for 90 minutes then concentrated to an oil. The residue was purified by silica gel column chromatography (ethyl acetate/hexanes, 1:8) to give 6,6-dimethyl-5,6,7,8-tetrahydroquinazolin-4(3H)-one (6.41 g, 90% yield) as a white solid. ¹H NMR (400 MHz, d₆-DMSO): 7.96 (s, 1H), 2.52 (t, 2H), 2.14 (s, 2H), 1.48 (t, 2H), 0.93 (s, 6H); MS (EI) for C₁₀H₁₄N₂O: 179 (MH⁺).

STEP 5: To 6,6-dimethyl-5,6,7,8-tetrahydroquinazolin-4(3H)-one (6.41 g, 36 mmol) in chloroform (10 mL) added phosphorus oxychloride (10 mL) and refluxed for 2 hours. The mixture was concentrated to an oil, then diluted with ethyl acetate (80 mL) and washed with saturated sodium carbonate (50 mL) and brine (25 mL). The solution was dried over anhydrous sodium sulfate, filtered and concentrated, then the residue purified by silica gel column chromatography (ethyl acetate/hexanes, 1:8) to give 4-chloro-6,6-dimethyl-5,6,7,8-tetrahydroquinazoline (5.3 g, 75% yield) as a yellow solid. ¹H NMR (400 MHz, CDCl₃): 8.72 (s, 1H), 2.52 (t, 2H), 2.14 (s, 2H), 1.48 (t 2H), 0.93 (s, 6H); MS (EI) for C₁₀H₁₃ClN₂: 197 (MH⁺).

Using analogous synthetic techniques and substituting with alternative starting reagents in step 1 or 2 the following reagents were prepared. Alternative starting materials were available commercially unless otherwise indicated.

4-chloro-6-methyl-6,7-dihydro-5H-cyclopenta[d]pyrimidine. Prepared according to the method of reagent preparation 3; using 4-methyl-2-oxo-cyclopentanecarboxylic acid methyl ester (J. Chem. Soc. Perkin Trans 1 1987, 7, 1485-8) in step 2. ¹H NMR (400 MHz, CDCl₃): 8.78 (s, 1H), 3.20 (m, 2H), 2.70 (m, 3H), 1.22 (d, 3H). GC/MS (EI) for C₈H₉ClN₂: 168 (M⁺).

4-chloro-6-cyclopropyl-5,6,7,8-tetrahydropyrido[4,3-d]pyrimidine. Prepared according to the method of reagent preparation 3 using 1-cyclopropyl-4-oxo-3-piperidinecarboxylic acid methyl ester (Heterocycles, 1999, 50(2), 867-874) in step 2. ¹H NMR (400 MHz, CDCl₃): 8.78 (s, 1H), 3.79 (s, 2H), 2.98 (m, 4H), 1.88 (m, 1H), 0.60 (m, 2H), 0.54 (m, 2H). MS (EI) for C₁₀H₁₂ClN₃: 210 (MH⁺).

4-chloro-6-cyclopropyl-6,7-dihydro-5H-pyrrolo[3,4-d]pyrimidine. Prepared according to the method of reagent preparation 3 using 1-cyclopropyl-4-oxo-3-pyrrolidinecarboxylic acid methyl ester in step 2. MS (EI) for C₉H₁₀ClN₃: 196 (MH⁺).

4-chloro-6-p-tolyl-6,7-dihydro-5H-pyrrolo[3,4-d]pyrimidine. Prepared according to the method of reagent preparation 3 using 1-(4-methylphenyl)-4-oxo-3-pyrrolidinecarboxilic acid ethyl ester in step 2. ¹H NMR (400 MHz, CDCl₃): 8.92 (s, 1H), 7.14 (d, 2H), 6.62 (d, 2H), 4.70 (m, 4H), 2.30 (s, 3H). MS (EI) for C₁₃H₁₂ClN₃: 246 (MH⁺).

4-chloro-7-methyl-7-phenyl-5,6,7,8-tetrahydroquinazoline. Prepared according to the method of reagent preparation 3 using 4-methyl-2-oxo-4-phenyl cyclohexanecarboxylic acid methyl ester (J. Org. Chem. 1991, 56(21), 6199-205) in step 1. MS (EI) for C₁₅H₁₅ClN₂: 259 (MH⁺).

4-chloro-5-phenyl-6,7-dihydro-5H-cyclopenta[d]pyrimidine: Synthesized according to the method of reagent preparation 3 using ethyl 2-oxo-5-phenylcyclopentanecarboxylate in step 2. MS (EI) for C₁₃H₁₁ClN₂: 231 (MH⁺).

4-chloro-7,7-dimethyl-5,6,7,8-tetrahydroquinazoline: Synthesized according to the method of reagent preparation 3 using ethyl 4,4-dimethyl-2-oxocyclohexanecarboxylate in step 2. ¹H NMR (400 MHz, CDCl₃): 8.91 (s, 1H), 2.90 (s, 2H), 2.88 (tr, 2H), 1.73 (tr, 2H), 1.07 (s, 6H); MS (EI) for C₁₀H₁₃ClN₂: 197 (MH⁺).

4′-chloro-7′,8′-dihydro-5′H-spiro[cyclopropane-1,6′-quinazoline]. Prepared according to the method of reagent preparation 3 using spiro[2.5]octan-6-one in step 1. ¹H NMR (400 MHz, CDCl₃) δ 8.73 (s, 1H), 3.00 (t, 2H), 2.63 (s, 2H), 1.69 (t, 2H), 0.52 (s, 4H); MS (EI) for C₁₀H₁₁ClN₂: 194 (M⁺).

4-chloro-6,6-difluoro-5,6,7,8-tetrahydroquinazoline. Synthesized according to the method of reagent preparation 3 using 4,4-difluorocyclohexanone in step 1. MS (EI) for C₈H₇ClF₂N₂: 204 (M⁺).

(R)-4-chloro-7-methyl-5,6,7,8-tetrahydroquinazoline. Synthesized according to the method of reagent preparation 3 using (R)-3-methylcyclohexanone in step 1. MS (EI) for C₉H₁₁ClN₂: 182 (M⁺).

4-chloro-2,6-dimethyl-5,6,7,8-tetrahydroquinazoline. Synthesized according to the method of reagent preparation 3 using 4-methylcyclohexanone in step 1 and 1,1-dimethoxy-N,N-dimethylethanamine in step 3. MS (EI) for C₁₀H₁₃ClN₂: 196 (M⁺).

4-chloro-6-ethyl-2-methyl-5,6,7,8-tetrahydroquinazoline. Synthesized according to the method of reagent preparation 3 using 4-ethylcyclohexanone in step 1 and 1,1-dimethoxy-N,N-dimethylethanamine in step 3. MS (EI) for C₁₁H₁₅ClN₂: 210 (M⁺).

4-chloro-7-(trifluoromethyl)-5,6,7,8-tetrahydroquinazoline. Synthesized according to the method of reagent preparation 3 using methyl 2-hydroxy-4-(trifluormethyl)cyclohex-1-enecarboxylate in step 2. MS (EI) for C₉H₈ClF₃N₂: 236 (M⁺).

(trans)-4-chloro-6,7-dimethyl-5,6,7,8-tetrahydroquinazoline. Synthesized according to the method of reagent preparation 3 using (trans) 3,4-dimethylcyclohexanone in step 1. MS (EI) for C₁₀H₁₃ClN₂: 196 (M⁺).

4-chloro-6-(trifluoromethyl)-5,6,7,8-tetrahydroquinazoline. Synthesized according to the method of reagent preparation 3 using 4-(trifluormethyl)cyclohexanone in step 1. MS (EI) for C₉H₈ClF₃N₂: 236 (M⁺).

(S)-4-chloro-7-methyl-5,6,7,8-tetrahydroquinazoline. Synthesized according to the method of reagent preparation 3 using (S)-3-methylcyclohexanone (US20060293364) in step 1. MS (EI) for C₉H₁₁ClN₂: 182 (M⁺).

4-chloro-5-(trifluoromethyl)-5,6,7,8-tetrahydroquinazoline. Synthesized according to the method of reagent preparation 3 using methyl 2-hydroxy-6-(trifluormethyl)cyclohex-1-enecarboxylate in step 2. MS (EI) for C₉H₈ClF₃N₂: 236 (M⁺).

4-chloro-7-vinyl-5,6,7,8-tetrahydroquinazoline. Synthesized according to the method of reagent preparation 3 using 3-vinylcyclohexanone (J. Med. Chem. 1987, 30, 1177-1186) in step 1. MS (EI) for C₁₀H₁₁ClN₂: 194 (M⁺).

4-chloro-8,8-dimethyl-5,6,7,8-tetrahydroquinazoline. Synthesized according to the method of reagent preparation 3 using 2,2-dimethylcyclohexanone in step 1. MS (EI) for C₁₀H₁₃ClN₂: 196 (M⁺).

4-chloro-6,6,7-dimethyl-5,6-dihydroquinazoline. Synthesized according to the method of reagent preparation 3 using 3,4,4-trimethylcyclohex-2-enone (J. Am. Chem. Soc. 1994, 116, 2902-2913) in step 1. MS (EI) for C₁₁H₁₃ClN₂: 208 (M⁺).

(S)-4-chloro-8-vinyl-6,7,8,9-tetrahydro-5H-cyclohepta[d]pyrimidine. Synthesized according to the method of reagent preparation 3 using (S)-3-vinylcycloheptanone (prepared using procedure for (S)-3-vinylcyclohexanone in Org. Lett. 2003, 5, 97-99, but starting with (Z)-cyclohept-2-enone) in step 1. MS (EI) for C₁₁H₁₃ClN₂: 208 (M⁺).

4-chloro-6,6-dimethyl-5,6-dihydroquinazoline. Synthesized according to the method of reagent preparation 3 using 4,4-dimethylcyclohex-2-enone in step 1. MS (ES) for C₁₀H₁₁ClN₂: 195 (MH⁺).

4-chloro-6,6,8-dimethyl-5,6-dihydroquinazoline. Synthesized according to the method of reagent preparation 3 using 2,4,4-trimethylcyclohex-2-enone in step 1. MS (EI) for C₁₁H₁₃ClN₂: 209 (MH⁺).

4-chloro-6,6,7,8-tetramethyl-5,6-dihydroquinazoline. Synthesized according to the method of reagent preparation 3 using 2,3,4,4-tetramethylcyclohex-2-enone (J. Org. Chem. 1981, 46, 1515-1521) in step 1. MS (EI) for C₁₂H₁₅ClN₂: 223 (MH⁺).

(S)-4-chloro-7-ethyl-5,6,7,8-tetrahydroquinazoline. Synthesized according to the method of reagent preparation 3 using (S)-3-ethylcyclohexanone (Tetrahedron: Asymmetry, 1997, 8, 1253-1257) in step 1. MS (EI) for C₁₀H₁₃ClN₂: 197 (MH⁺).

### Reagent Preparation 4

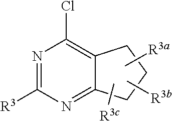

Step 1: A solution of methyl 4-methyl-2-oxocyclopentanecarboxylate (0.42 g, 2.69 mmol), 2-methyl-2-thiopseudourea sulfate (1.10 g, 7.9 mmol) and potassium hydroxide (0.50 g, 8.9 mmol) in water (12 mL) was stirred at 25° C. for 30 minutes, and then heated to reflux for 4 hours. The reaction was cooled to 0° C. by adding ice and a precipitate was formed. The solid product was removed by filtration and the filter cake dried to give 6-methyl-2-(methylthio)-6,7-dihydro-3H-cyclopenta[d]pyrimidin-4(5H)-one (0.19 g, 43% yield) as a white solid. ¹H NMR (400 MHz, d6-DMSO): 2.87 (m, 2H), 2.53 (s, 3H), 2.37 (m, 2H), 2.28 (s, 3H), 1.49 (m, 1H), 1.02 (d, 3H).

Step 2: A solution of 6-methyl-2-(methylthio)-6,7-dihydro-3H-cyclopenta[d]pyrimidin-4(5H)-one (0.19 g, 0.97 mmol) in phosphorous oxychloride (5.0 mL) was heated to 95° C. for 1 hour. After cooling the reaction was concentrated, and the residue dissolved in ethyl acetate (50 mL) and washed with cold water (25 mL), 0.1 M aqueous sodium hydroxide (25 mL) and brine (20 mL). The organic phase was dried over anhydrous sodium sulfate, filtered and concentrated. The residue was chromatographed on silica gel (diethyl ether/hexanes, 1:10) and the product containing fractions concentrated. The residue thus obtained was purified further by preparative reverse phase HPLC (0.1% aqueous ammonium acetate-acetonitrile to give 4-chloro-6-methyl-2-(methylthio)-6,7-dihydro-5H-cyclopenta[d]pyrimidine (25 mg, 12% yield) as an oil. ¹H NMR (400 MHz, d6-DMSO): 3.12 (m, 2H), 2.61 (m, 2H), 2.56 (s, 3H), 1.25 (m, 1H), 1.18 (d, 3H); MS (EI) for C₉H₁₁ClN₂S: 215 (MH⁺).

Using analogous synthetic techniques and substituting with alternative starting reagents the following reagents were prepared.

4-chloro-2-(methylthio)-6,7-dihydro-5H-cyclopenta[d]pyrimidine. Synthesized according to the method of reagent preparation 4 by replacement of step 1 with 1,2,3,5,6,7-hexahydro-2-thioxo-4H-cyclopentapyrimidin-4-one S-alkylation with iodomethane and proceeding to step 2. ¹H NMR (400 MHz, CDCl₃): 3.00 (tr, 2H), 2.92 (x, 2H), 2.56 (s, 3H), 2.14 (m, 2H).

2-(benzylthio)-4-chloro-6,7-dihydro-5H-cyclopenta[d]pyrimidine. Synthesized according to the method of reagent preparation 4 by replacement of step 1 with 1,2,3,5,6,7-hexahydro-2-thioxo-4H-cyclopentapyrimidin-4-one S-alkylation with benzyl bromide and proceeding to step 2. ¹H NMR (400 MHz, CDCl₃): 7.43 (d, 2H), 7.27 (tr, 2H), 7.22-7.18 (m, 1H), 4.38 (s, 2H), 2.95 (tr, 2H), 2.86 (tr, 2H), 2.08 (m, 2H).

4-chloro-2-(ethylthio)-6,7-dihydro-5H-cyclopenta[d]pyrimidine. Synthesized according to the method of reagent preparation 4 by replacement of step 1 with 1,2,3,5,6,7-hexahydro-2-thioxo-4H-cyclopentapyrimidin-4-one S-alkylation with iodoethane and proceeding to step 2. ¹H NMR (400 MHz, CDCl₃): 3.08 (q, 2H), 2.93 (tr, 2H), 2.86 (tr, 2H), 2.08 (m, 2H), 1.32 (tr, 3H).

### Reagent Preparation 5

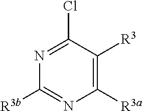

STEP 1: A solution of ethyl 4-methyl-3-oxopentanoate (3.0 g, 19.0 mmol) and potassium carbonate (7.86 g, 56.9 mmol) in THF (40 mL) was stirred at room temperature for 3 h under N₂ (g). The mixture was cooled to 0° C. and methyl iodide (3.23 g, 22.8 mmol) was added dropwise over 5 min. The reaction mixture was allowed to warm to room temperature and stirred for 16 h. Subsequent filtration and concentration provided ethyl 2,4-dimethyl-3-oxopentanoate (2.89 g, 89% yield) as a clear yellow oil that was used without further purification. MS (EI) for C₉H₁₆O₃: 172 (MH⁺).

STEP 2: To anhydrous ethanol (110 mL) was added sodium metal (1.16 g, 50.4 mmol) and the mixture was stirred until dissolution was complete. To this solution was added thiourea (1.79 g, 23.5 mmol) and ethyl 2,4-dimethyl-3-oxopentanoate (2.89 g, 16.8 mmol). The reaction mixture was stirred at 85° C. for 20 h then cooled and concentrated. The residue was diluted with water, the pH adjusted to 4 with 1 N hydrochloric acid then extracted with ethyl acetate (3×80 mL). The combined organic layers were washed with brine, dried over anhydrous sodium sulfate, filtered and concentrated to provide 6-isopropyl-5-methyl-2-thioxo-2,3-dihydropyrimidin-4(1H)-one (2.40 g, 78% yield) as a tan solid that was used without further purification. C₈H₁₂N₂OS: 185 (MH⁺).

STEP 3: To a solution of 30% hydrogen peroxide (12 mL) and water (23 mL) was slowly added 6-isopropyl-5-methyl-2-thioxo-2,3-dihydropyrimidin-4(1H)-one (1.0 g, 5.4 mmol). The reaction mixture was stirred at 70° C. for 3 h. After cooling to room temperature, saturated sodium carbonate was slowly added until the pH reached 10. To this mixture was slowly added a 1 M solution of sodium thiosulfate until residual peroxide was quenched, whereupon the aqueous solution was concentrated to dryness. The residue was suspended in chloroform (100 mL), filtered to remove inorganic salts and the filtrate concentrated to provide 6-isopropyl-5-methylpyrimidin-4-ol (0.25 g, 30% yield) as a white solid that was used without further purification. MS (EI) for C₈H₁₂N₂O: 153 (MH⁺).

STEP 4: To 6-isopropyl-5-methylpyrimidin-4-ol (0.25 g, 1.6 mmol) was added neat phosphorous oxychloride (5 mL) and the mixture stirred at 70° C. for 3 h. After cooling to room temperature the solution was concentrated, diluted with water then neutralized by portionwise addition of saturated sodium carbonate solution. The aqueous mixture was extracted with ethyl acetate and the organic solution washed with brine then dried over anhydrous sodium sulfate. Filtration and concentration provided 4-chloro-6-isopropyl-5-methylpyrimidine (30 mg, 11% yield) as a brown oil that was used without further purification. MS (EI) for C₈H₁₁ClN₂: 170 (MH⁺).

Using analogous synthetic techniques and substituting with alternative starting reagents in step 1 the following reagents were prepared.

4-chloro-5-(cyclopropylmethyl)-6-methylpyrimidine. Synthesized according to the method of reagent preparation 5 using methyl 3-oxobutanoate and (bromomethyl)cyclopropane in step 1. MS (EI) for C₉H₁₁ClN₂: 182 (MH).

4-chloro-5-(4-chlorobenzyl)-6-methylpyrimidine. Synthesized according to the method of reagent preparation 5 using methyl 3-oxobutanoate and 1-(bromomethyl)-4-chlorobenzene in step 1. MS (EI) for C₁₂H₁₀Cl₂N₂: 254 (MH⁺).

4-chloro-5-(3,5-difluorobenzyl)-6-methylpyrimidine. Synthesized according to the method of reagent preparation 5 using methyl 3-oxobutanoate and 1-(bromomethyl)-3,5-difluorobenzene in step 1. MS (EI) for C₁₂H₉ClF₂N₂: 255 (MH⁺).

4-chloro-6-methyl-5-(3-(trifluoromethyl)benzyl)pyrimidine. Synthesized according to the method of reagent preparation 5 using methyl 3-oxobutanoate and 1-(chloromethyl)-3-(trifluoromethyl)benzene in step 1. MS (EI) for C₁₃H₁₀ClF₃N₂: 287 (MH⁺).

4-chloro-5-(1-(3-fluorophenyl)ethyl)-6-methylpyrimidine. Synthesized according to the method of reagent preparation 5 using methyl 3-oxobutanoate and 1-(3-fluorophenyl)ethyl methanesulfonate in step 1. MS (EI) for C₁₃H₁₂ClFN₂: 251 (MH⁺).

4-chloro-5-(4-chloro-3-fluorobenzyl)-6-methylpyrimidine. Synthesized according to the method of reagent preparation 5 using methyl 3-oxobutanoate and 4-(bromomethyl)-1-chloro-2-fluorobenzene in step 1. MS (EI) for C₁₂H₉Cl₂FN₂: 272 (MH⁺).

4-chloro-5-(4-fluorobenzyl)-6-methylpyrimidine. Synthesized according to the method of reagent preparation 5 using methyl 3-oxobutanoate and 1-(bromomethyl)-4-fluorobenzene in step 1. MS (EI) for C₁₂H₁₀ClFN₂: 237 (MH⁺).

4-chloro-5-(2-fluorobenzyl)-6-methylpyrimidine. Prepared according to the method of reagent preparation 5 by using methyl 3-oxobutanoate and 1-(bromomethyl)-2-fluorobenzene in step 1. ¹H NMR (400 MHz, CDCl₃): 8.79 (1H), 7.28 to 7.12 (m, 1H), 7.14 to 6.97 (m, 2H), 6.82 (dd, 1H), 4.19 (s, 2H), 2.47 (s, 3H), GC-MS for C₁₂H₁₀ClFN₂: 236 (M⁺).

4-chloro-5-ethyl-6-isopropylpyrimidine. Prepared according to reagent preparation 5 by using ethyl isobutyrylacetate and iodoethane in step 1. MS (EI) for C₉H₁₃ClN₂: 184 (M⁺).

5-benzyl-4-chloro-6-methylpyrimidine. Prepared according to reagent preparation 5 by using ethyl 2-benzylacetoacetate in step 2. MS (EI) for C₁₂H₁₁ClN₂: 219 (MH⁺).

4-chloro-6-ethyl-5-methyl-pyrimidine. Prepared according to reagent preparation 5 by using methyl 3-oxopentanoate in step 1. ¹H NMR (400 MHz, CDCl₃): 8.74 (s, 1H), 2.85 (q, 2H), 2.39 (s, 3H), 1.30 (t, 3H); MS (EI) for C₇H₉ClN₂: 158 (MH⁺).

4-chloro-5,6,7,8-tetrahydroquinazoline. Synthesized according to the method of reagent preparation 5 using ethyl 2-oxocyclohexanecarboxylate in step 2. ¹H NMR (400 MHz, CDCl₃): 8.7 (s, 1H), 2.90 (m, 2H), 2.78 (m, 2H), 1.88 (m, 4H). MS (EI) for C₈H₉ClN₂: 169 (MH⁺).

4-chloro-5,6-diethyl-pyrimidine. Prepared according to reagent preparation 5 by using methyl 3-oxopentanoate and iodoethane in step 1.

4-chloro-6-methyl-5-(1-methylethyl)-pyrimidine. Prepared according to reagent preparation 5 by using methyl 3-oxobutanoate and 2-iodopropane in step 1. ¹H NMR (400 MHz, DMSO-d₆): 8.70 (s, 1H), 3.49 (h, 1H), 2.60 (s, 3H), 1.34 (d, 6H); MS (EI) for C₈H₁₁ClN₂: 171 (MH⁺).

4-chloro-5-isobutyl-6-methylpyrimidine. Prepared according to reagent preparation 5 by using methyl 3-oxobutanoate and 1-iodo-2-methylpropane in step 1. MS (EI) for C₉H₁₃ClN₂: 184 (M⁺).

5-benzyl-4-chloro-6-ethylpyrimidine. Prepared according to reagent preparation 5 by using methyl 3-oxopentanoate and benzyl bromide in step 1. ¹H NMR (400 MHz, CDCl₃): 8.83 (s, 1H), 7.27 (m, 3H), 7.08 (m, 2H), 4.22 (s, 2H), 2.79 (q, 2H), 1.20 (t, 3H); MS (EI) for C₁₃H₁₃ClN₂: 234 (MH⁺).

4-chloro-5-(3-fluorobenzyl)-6-methylpyrimidine. Prepared according to reagent preparation 5 by using methyl 3-oxobutanoate and 3-fluorobenzylbromide in step 1. MS (EI) for C₁₂H₁₀ClFN₂: 237 (MH⁺).

4-chloro-5-(3-chlorobenzyl)-6-methylpyrimidine. Prepared according to reagent preparation 5 by using methyl 3-oxobutanoate and 3-chlorobenzylbromide in step 1. MS (EI) for C₁₂H₁₀Cl₂N₂: 253 (MH⁺).

4-chloro-6-methyl-5-phenoxy-pyrimidine. Prepared according to reagent preparation 5 by using ethyl 3-oxo-2-phenoxybutanoate in step 2. MS (EI) for C₁₁H₉ClN₂O: 221 (MH⁺).

4-chloro-6-methyl-5-(1-phenylethyl)pyrimidine. Prepared according to reagent preparation 5 by using methyl 3-oxobutanoate and (1-bromoethyl)benzene in step 1. MS (EI) for C₁₃H₁₃ClN₂: 233 (MH⁺).

4-chloro-5-(2-chlorobenzyl)-6-methylpyrimidine. Prepared according to reagent preparation 5 by using methyl 3-oxobutanoate and 2-chlorobenzyl bromide in step 1.

4-chloro-6-methyl-5-(4-methylbenzyl)pyrimidine. Prepared according to reagent preparation 5 by using methyl 3-oxobutanoate and 4-methylbenzyl bromide in step 1. ¹H NMR (400 MHz, CDCl₃): 8.76 (s, 1H), 7.10 (d, 2H), 6.99 (d, 2H), 4.15 (s, 2H), 2.50 (s, 3H), 2.32 (s, 3H); MS (EI) for C₁₃H₁₃ClN₂: 233 (MH⁺).

4-chloro-5-(4-methoxybenzyl)-6-methylpyrimidine. Prepared according to reagent preparation 5 by using methyl 3-oxobutanoate and 4-methoxybenzyl bromide in step 1. ¹H NMR (400 MHz, CDCl₃): 8.76 (s, 1H), 7.02 (d, 2H), 6.83 (d, 2H), 4.13 (s, 2H), 3.78 (s, 3H), 2.51 (s, 3H); MS (EI) for C₁₃H₁₃ClN₂O: 249 (MH⁺).

4-chloro-5-(3-methoxybenzyl)-6-methylpyrimidine. Prepared according to reagent preparation 5 by using methyl 3-oxobutanoate and 3-methoxybenzyl bromide in step 1. ¹H NMR (400 MHz, DMSO-d₆): 8.81 (s, 1H), 7.22 (m, 1H), 6.81 (m, 1H), 6.70 (s, 1H), 6.63 (d, 1H), 4.17 (s, 2H), 3.71 (s, 3H), 2.47 (s, 3H); MS (EI) for C₁₃H₁₃ClN₂O: 249 (MH⁺).

4-chloro-6-methyl-5-(3-methylbenzyl)pyrimidine. Prepared according to reagent preparation 5 by using methyl 3-oxobutanoate and 3-methylbenzyl bromide in step 1. ¹H NMR (400 MHz, CDCl₃): 8.77 (s, 1H), 7.18 (m, 1H), 7.05 (d, 1H), 6.88 (m, 2H), 4.16 (s, 2H), 2.50 (s, 3H), 2.31 (s, 3H); MS (EI) for C₁₃H₁₃ClN₂: 233 (MH⁺).

5-benzyl-4-chloropyrimidine. Prepared according to reagent preparation 5 by using ethyl 2-benzyl-3-hydroxyacrylate (J. Am. Chem. Soc. 1974, 96, 2121-2129) in step 2. MS (EI) for C₁₁H₉₃ClN₂: 205 (MH⁺).

4-chloro-5-(3-chloro-5-fluorobenzyl)-6-methylpyrimidine. Prepared according to reagent preparation 5 by using methyl 3-oxobutanoate and 3-chloro-5-fluorobenzyl bromide in step 1. MS (EI) for C₁₂H₉Cl₂FN₂: 271 (MH⁺).

4-chloro-5-(2-methoxybenzyl)-6-methylpyrimidine. Prepared according to reagent preparation 5 by using methyl 3-oxobutanoate and 2-methoxylbenzyl bromide in step 1. ¹H NMR (400 MHz, methanol-d₄): 8.71 (s, 1H), 7.23 (m, 1H), 6.98 (d, 1H), 6.83 (m, 1H), 6.71 (d, 1H), 4.16 (s, 2H), 3.85 (s, 3H), 2.45 (s, 3H).

4-chloro-6-methyl-5-(2-methylbenzyl)pyrimidine. Prepared according to reagent preparation 5 by using methyl 3-oxobutanoate and 2-methylbenzyl bromide in step 1. ¹H NMR (400 MHz, methanol-d₄): 8.77 (s, 1H), 7.23 (d, 1H), 7.12 (m, 1H), 7.03 (m, 1H), 6.45 (d, 1H), 4.16 (s, 2H), 2.43 (s, 3H), 2.42 (s, 3H).

4-chloro-5-(3,4-difluorobenzyl)-6-methylpyrimidine. Prepared according to reagent preparation 5 by using methyl 3-oxobutanoate and 3,4-difluorobenzyl bromide in step 1. MS (EI) for C₁₂H₉ClF₂N₂: 255 (MH⁺).

4-chloro-6-methyl-5-(4-(trifluoromethyl)benzyl)pyrimidine. Prepared according to reagent preparation 5 by using methyl 3-oxobutanoate and 1-(chloromethyl)-4-(trifluoro-methyl)benzene in step 1. MS (EI) for C₁₃H₁₀ClF₃N₂: 287 (MH⁺).

5-benzyl-4-chloro-6-(trifluoromethyl)pyrimidine. Prepared according to reagent preparation 5 by using ethyl 4,4,4-trifluoroacetoacetate and benzyl bromide in step 1. MS (EI) for C₁₂H₈ClF₃N₂: 272 (M⁺).

4-chloro-6,6-dimethyl-6,7-dihydro-5H-cyclopenta[d]pyrimidine. Synthesized according to the method of reagent preparation 5 using ethyl 4,4-dimethyl-2-oxocyclopentanecarboxylate in step 2. MS (EI) for C₉H₁₁ClN₂: 183 (MH⁺).

### Reagent Preparation 6

### 6-chloro-5-methyl-N-phenylpyrimidin-4-amine

STEP 1: To a mixture of 4,6-dichloro-5-methylpyrimidine (2.27 g, 13.9 mmol) and aniline (1.0 g, 10.7 mmol) in isopropanol (15 mL) was added concentrated aqueous hydrochloric acid (1.5 mL) and heated to reflux for 2.5 h. The mixture was then concentrated and the residue triturated with ethyl acetate:isopropanol 4:1. The solid was collected by filtration and washed with additional ethyl acetate:isopropanol 4:1 then dried to give 6-chloro-5-methyl-N-phenylpyrimidin-4-amine (2.0 g, 67% yield). ¹H NMR (400 MHz, d₆-DMSO): 8.85 (s, 1H), 8.26 (s, 1H), 7.60 (d, 2H), 7.35 (tr, 2H), 7.11 (tr, 1H), 2.31 (s, 3H). MS (EI) for C₁₁H₁₀ClN₃: 220 (MH⁺).

### Reagent Preparation 8

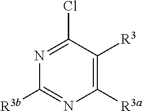

STEP 1: To a suspension of potassium tert-butoxide (10.6 g, 95.0 mmol) in tetrahydrofuran (100 mL) were added methyl acetoacetate (10.0 g, 86.0 mmol) and tert-butanol (0.83 mL, 8.6 mmol) at room temperature. The resulting solution was stirred for 1 h, and then 4-fluorobenzylbromide (11.2 mL, 90 mmol) was added. The reaction mixture was stirred at room temperature for 18 h, and then partitioned between water and ethyl acetate. The aqueous layer was extracted with ethyl acetate (3×), the combined organic extracts were washed with brine, dried over sodium sulfate, filtered and concentrated. Column chromatography of the residue on silica (5-20% ethyl acetate in hexanes) gave methyl 2-(4-fluorobenzyl)-3-oxobutanoate (14.5 g, 75% yield) as a colorless oil which was used in the next step without further purification.

STEP 2: To a suspension of acetamidine hydrochloride (0.54 g, 5.71 mmol) in methanol (8 mL) was added a 30% solution of sodium methoxide in methanol (1.1 mL, 5.7 mmol), and the resulting solution was stirred at room temperature for 45 min. Then, a solution of methyl 2-(4-fluorobenzyl)-3-oxobutanoate (0.80 g, 3.57 mmol) in methanol (3 mL) was added dropwise, and the resulting mixture was stirred at room temperature for 22 h. Water (100 mL) was added, and the mixture was extracted with chloroform (4×50 mL). The combined organic extracts were dried over sodium sulfate, filtered and concentrated to provide 5-(4-fluorobenzyl)-2,6-dimethylpyrimidin-4-ol (0.74 g, 89% yield) as a colorless solid. ¹H NMR (400 MHz, methanol-d₄): 7.21 (m, 2H), 6.96 (m, 2H), 3.84 (s, 2H), 2.35 (s, 3H), 2.25 (s, 3H); MS (EI) for C₁₃H₁₃FN₂O: 233 (MH⁺).

STEP 3: A solution of 5-(4-fluorobenzyl)-2,6-dimethylpyrimidin-4-ol (730 mg, 3.14 mmol) in phosphorus oxychloride (10 mL) was stirred at 60° C. for 90 min. The reaction mixture was concentrated and ethyl acetate (50 mL) was added to the residue. The organic solution was washed with saturated sodium bicarbonate (50 mL), water (50 mL), and brine (50 mL), dried over sodium sulfate, filtered and concentrated. Column chromatography of the residue on silica (5-40% ethyl acetate in hexanes) afforded 4-chloro-5-(4-fluorobenzyl)-2,6-dimethylpyrimidine (527 mg, 67% yield) as a colorless solid. ¹H NMR (400 MHz, CDCl₃): 7.21 (m, 2H), 6.98 (m, 2H), 4.12 (s, 2H), 2.67 (s, 3H), 2.45 (s, 3H); MS (EI) for C₁₃H₁₂ClFN₂: 250 (M⁺).

Using analogous synthetic techniques and substituting with alternative starting reagents in step 1 the following reagents were prepared.

4-Chloro-7-methyl-5,6,7,8-tetrahydroquinazoline. Prepared according to the method of reagent preparation 8 by using ethyl 4-methyl-2-oxocyclohexanecarboxylate and formamidine formate in step 2. GC-MS for C₉H₁₁ClN₂: 182 (M⁺).

4-Chloro-6-ethyl-5,6,7,8-tetrahydroquinazoline. Prepared according to the method of reagent preparation 8 by using methyl 5-ethyl-2-oxocyclohexanecarboxylate and formamidine formate in step 2. GC-MS for C₁₀H₁₃ClN₂: 196 (M⁺).

4-Chloro-5-ethyl-2,6-dimethylpyrimidine. Synthesized according to the method of reagent preparation 8 by using ethyliodide in step 1. MS (EI) for C₈H₁₁ClN₂: 171 (MH⁺).

4-Chloro-5-(cyclopropylmethyl)-2,6-dimethylpyrimidine. Synthesized according to the method of reagent preparation 8 by using cyclopropylmethylbromide in step 1. MS (EI) for C₁₀H₁₃ClN₂: 197 (MH⁺).

4-Chloro-2,6,6-dimethyl-5,6,7,8-tetrahydroquinazoline. Synthesized according to the method of reagent preparation 8 by using methyl 5,5-dimethyl-2-oxocyclohexanecarboxylate in step 2. MS (EI) for C₁₁H₁₅ClN₂: 211 (MH⁺).

4-Chloro-6,6-dimethyl-2-(pyridin-2-yl)-5,6,7,8-tetrahydroquinazoline. Synthesized according to the method of reagent preparation 8 by using 2-hydroxy-5,5-dimethylcyclohex-1-enecarboxylate and picolinimidamide hydrochloride in step 2. MS (ES) for C₁₅H₁₆ClN₃: 274 (MH⁺).

2-(4-chloro-6,6-dimethyl-5,6,7,8-tetrahydroquinazolin-2-yl)propan-2-ol. Synthesized according to the method of reagent preparation 8 using 2-hydroxy-5,5-dimethylcyclohex-1-enecarboxylate and 2-hydroxy-2-methylpropanimidamide hydrochloride in step 2. MS (ES) for C₁₃H₁₉ClN₂: 255 (MH⁺).

4-chloro-2,6-dimethyl-5-(1-methylethyl)pyrimidine. Synthesized according to the method of reagent preparation 8 by using 2-iodopropane in step 1. MS (EI) for C₉H₁₃ClN₂: 185 (MH⁺).

(7S)-4-chloro-7-ethyl-2-methyl-5,6,7,8-tetrahydroquinazoline. Synthesized according to the method of reagent preparation 8 by using methyl (4S)-4-ethyl-2-oxocyclohexanecarboxylate (reagent preparation 3) in step 2. MS (EI) for C₁₁H₁₅ClN₂: 211 (MH⁺).

4-chloro-6,6-dimethyl-2-(2-pyrrolidin-1-ylethyl)-5,6,7,8-tetrahydroquinazoline. Synthesized according to the method of reagent preparation 8 by using 1-pyrrolidinepropanimidamide in step 2. MS (EI) for C₁₆H₂₄ClN₃: 294 (MH⁺).

### Reagent Preparation 9

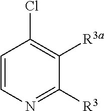

STEP 1: To a solution of phenylmethyl 2-methyl-4-oxo-3,4-dihydropyridine-1(2H)-carboxylate (J. Bioorg. Med. Chem. 2007, 1106-1116) (2.4 g, 9.78 mmol) in THF (35 mL) was added dropwise a 1M solution of lithium bis(trimethylsilyl)amide in THF (11 mL) at −78° C. The solution was warmed up to 0° C., stirred at this temperature for 1 h, then cooled again to −78° C. 3-Fluorobenzadehyde (1.3 mL, 12.7 mmol) was added in one portion. The reaction was stirred for 4 h while allowing it to slowly warm up to 0° C. Then, saturated ammonium chloride (20 mL) was added, and the layers were separated. The aqueous layer was extracted with ethyl acetate (2×20 mL) and the combined organic layers were washed with saturated sodium chloride (50 mL), dried over sodium sulfate, filtered and concentrated. Column chromatography on silica (gradient 20 to 100% ethyl acetate in hexanes) afforded phenylmethyl 3-[(3-fluorophenyl)(hydroxy)methyl]-2-methyl-4-oxo-3,4-dihydropyridine-1(2H)-carboxylate (2.4 g, 66% yield) as mixture of diastereomers. MS (EI) for C₂₁H₂₀FNO₄: 370.1 (MH⁺).

STEP 2: Mesyl chloride (0.31 mL, 3.97 mmol) was added in one portion to a solution of phenylmethyl 3-[(3-fluorophenyl)(hydroxy)methyl]-2-methyl-4-oxo-3,4-dihydropyridine-1(2H)-carboxylate (0.73 g, 1.98 mmol) in anhydrous pyridine (5 mL) at 0° C. The reaction mixture was warmed up to room temperature and stirred for 1 h. Water (5 mL) and ethyl acetate (5 mL) were added, the layers were separated, and the aqueous layer was extracted with ethyl acetate (3×5 mL). The combined organic layers were washed with saturated sodium chloride (15 mL) dried over sodium sulfate, filtered and concentrated to afford phenylmethyl 3-{(3-fluorophenyl)[methylsulfonyl)oxy]methyl}-2-methyl-4-oxo-3,4-dihydropyridine-1(2H)-carboxylate. MS (EI) for C₂₂H₂₂FNO₆S: 448.1 (MH⁺).

STEP 3: Phenylmethyl 3-{(3-fluorophenyl)[methylsulfonyl)oxy]methyl}-2-methyl-4-oxo-3,4-dihydropyridine-1(2H)-carboxylate from step 2 was dissolved in THF (30 mL) and potassium tert-butoxide (1.11 g, 9.9 mmol) was added in one portion. After 15 min the reaction mixture was quenched with saturated ammonium chloride (20 mL). The layers were separated and the aqueous layer was extracted with 5:1 chloroform/isopropanol (3×20 mL). The combined organic layers were dried over sodium sulfate, filtered and concentrated. Column chromatography in silica (10% methanol in dichloromethane) afforded 34(3-fluorophenyl)methyl)-2-methylpyridin-4(1H)-one (0.230 g, 53% for two steps) ¹H NMR (400 MHz, CDCl₃): 7.30 (d, 1H), 7.18-7.13 (m, 1H), 6.97 (d, 1H), 6.87-6.79 (m, 2H), 6.35 (d, 1H), 3.91 (s, 2H), 2.22 (s, 3H). MS (EI) for C₁₃H₁₂FNO: 218.1 (MH⁺).

STEP 4: A solution of 3-[(3-fluorophenyl)methyl)-2-methylpyridin-4(1H)-one (0.07 g, 0.32 mmol) in phosphorous oxychloride (3 mL) was heated to 55° C. for 16 h. Then the solution was cooled to room temperature and concentrated. The remaining residue was dissolved in ethyl acetate (10 mL), washed with 5% sodium bicarbonate (2×5 mL), and saturated sodium chloride (5 mL), dried over sodium sulfate, filtered and concentrated to afford 4-chloro-3-[(3-fluorophenyl)methyl]-2-methylpyridine. ¹H NMR (400 MHz, CDCl₃): 8.33 (d. 1H), 7.30-7.23 (m, 2H), 6.92-6.85 (m, 2H), 6.76 (d, 1H), 4.22 (s, 2H), 2.54 (s, 3H). MS (EI) for C₁₃H₁₁ClF: 236.0 (MH⁺).

Using analogous synthetic techniques and substituting with alternative starting reagents in step 1 the following reagents were prepared.

3-benzyl-4-chloro-2-methylpyridine. Synthesized according to the method of reagent preparation 9 using benzaldehyde in step 1. ¹H NMR (400 MHz, CDCl₃): 8.30 (d, 1H), 7.29-7.19 (m, 4H), 7.08 (d, 2H), 4.22 (s, 2H), 2.51 (s, 3H); MS (EI) for C₁₃H₁₂ClN: 218 (MH⁺).

4-chloro-3-(4-fluorobenzyl)-2-methylpyridine. Synthesized according to the method of reagent preparation 9 using 4-fluorobenzaldehyde in step 1. ¹H NMR (400 MHz, CDCl₃): 8.32 (d, 1H), 7.29 (d, 1H), 7.05-6.95 (m, 4H), 4.19 (s, 2H), 2.54 (s, 3H); MS (EI) for C₁₃H₁₁ClFN: 236 (MH⁺).

### Reagent Preparation 10

STEP 1: To a solution of ethyl 3-bromobutanoate (6.0 mL, 42 mmol) in N,N-dimethylformamide (20 mL) at 0° C. was added piperidine (8.0 mL, 80 mmol) and the mixture was warmed to room temperature then stirred 16 h. The reaction mixture was diluted with ethyl acetate (200 mL) and washed with a solution of brine and 2.0M aqueous sodium hydroxide (4:1 v/v). The organic phase was then dried over anhydrous sodium sulfate, filtered and concentrated to give ethyl 4-piperidin-1-ylbutanoate (6.8 g, 81% yield) as brown oil. MS (EI) for C₁₁H₂₁NO₂: 200 (MH⁺)

Step 2: To a solution of potassium hydroxide (11 g, 0.20 mol) in water (40 mL) was added a solution of ethyl 4-piperidin-1-ylbutanoate (6.8 g, 34 mmol) in ethanol (30 mL) and the mixture was stirred at 35° C. for 2 hours. The reaction was quenched by dropwise addition of 37% aqueous hydrochloric acid (15 mL) and the mixture was concentrated then dried under vacuum. The residue was suspended in chloroform (100 mL) followed by addition of catalytic N,N-dimethylformamide (0.2 mL) then dropwise addition of oxalyl chloride (15 mL, 170 mmol) and the mixture was stirred at 25° C. for 18 hours. The reaction mixture was concentrated to afford crude 4-piperidin-1-ylbutanoyl chloride hydrochloride. To a suspension of the 4-piperin-1-ylbutanoyl chloride hydrochloride (ca. 40 mmol) and 2-methyl-2-thiopseudourea sulfate (5.6 g, 20 mmol) in acetonitrile (100 mL) was added triethylamine (20 mL, 0.27 mol) in portions while cooling in an ice bath. The reaction was then allowed to warm to 25° C. over 1 h. The reaction mixture was filtered through Celite with an acetonitrile wash (100 mL). The filtrate was concentrated to afford methyl N,N′-bis-(4-piperidin-1-ylbutanoyl)imidothiocarbamate (10.6 g, 79% yield) as a brown oil that was used without further purification. MS (EI) for C₂₀H₃₆N₄O₂S: 397 (MH⁺).

Using analogous synthetic techniques and substituting with alternative starting reagents bis[2-(methoxy)ethoxy][(methylthio)methylidene]biscarbamate was prepared according to the method of reagent preparation 10 using 2-methoxyethyl chloroformate in step 2. MS (EI) for C₁₀H₁₈N₂O₆S: 295 (MH⁺).

### Reagent Preparation 11

STEP 1: To a solution of 6-bromo-2-methyl-1H-imidazo[4,5-b]pyridine (3.40 g, 16.0 mmol) and diisopropylethylamine (6.5 mL, 65 mmol) in N,N-dimethylformamide (20 mL) cooled in an ice bath was added dropwise isobutyl chloroformate (2.51 mL, 19.2 mmol) and the mixture was warmed to room temperature. After 1 hour the reaction was diluted with ethyl acetate (80 mL) and washed with water (60 mL), 10% aqueous citric acid (40 mL) and brine (20 mL). The organic phase was dried over anhydrous sodium sulfate, filtered and concentrated to a slurry. The residue was triturated diethyl ether (100 mL) and the solid isolated by filtration to give isobutyl 6-bromo-2-methyl-1H-imidazo[4,5-b]pyridine-1-carboxylate (2.3 g, 46% yield). MS (EI) for C₁₂H₁₄BrN₃O₂: 313 (MH⁺).

Using analogous synthetic techniques and substituting with alternative starting reagents in step 1 isobutyl 2-(4-bromophenyl)-1H-imidazole-1-carboxylate was prepared according to the method of reagent preparation 11 using 2-(4-bromophenyl)-1H-imidazole and isobutyl chloroformate in step 1. MS (EI) for C₁₄H₁₅BrN₂O₂: 324 (MH⁺).

Isobutyl 6-bromo-1H-benzo[d]imidazole-1-carboxylate. Prepared according to the method of reagent preparation 11 using 5-bromo-1H-benzo[d]imidazole in step 1. MS (EI) for C₁₂H₃BrN₂O₂: 297/299 (MH⁺).

### Reagent Preparation 12

### 5-Bromo-1-ethyl-1H-benzimidazole

5-bromo-1-ethyl-1H-benzimidazole was prepared in 3 steps from 1,4-dibromo-2-nitrobenzene according to the method described in (Bioorg. and Med. Chem. Lett. 2003, 13, 2485-2488). MS (EI) for C₉H₉BrN₂: 226 (MH⁺).

### Reagent Preparation 13

### N-(5-bromothiazolo[5,4-b]pyridin-2-yl)benzamide

STEP 1: To a solution of ammonium thiocyanate (0.4 g, 5.0 mmol) in acetone (5 mL) was slowly added benzoyl chloride (0.6 mL, 5.0 mmol) and the suspension was heated to reflux for ten minutes. A solution of 6-bromo-2-chloro-3-pyridinamine (1.0 g, 4.8 mmol) in acetone (10 mL) was then added and the reaction mixture was refluxed for one hour. After cooling to room temperature the mixture was poured into water and partitioned with ethyl acetate (250 mL). The layers were separated and the aqueous layer was further extracted with ethyl acetate (2×, 100 mL). The combined organic layers were washed with brine (2×, 100 mL), dried over sodium sulfate, filtered and concentrated intil a suspension formed. The white solid was collected by filtration to give N-(6-bromo-2-chloropyridin-3-ylcarbamothioyl)benzamide (1.6 g, 89%). ¹H NMR (400 MHz, d₆-DMSO): 12.62 (br s, 1H), 12.00 (br s, 1H), 8.37 (d, 1H), 8.00 (2d, 2H), 7.79 (d, 1H), 7.69 (t, 1H), 7.57 (t, 2H). MS (EI) for C₁₃H₉BrClN₃OS: 370 (MH⁺).

STEP 2: A solution of N-(6-bromo-2-chloropyridin-3-ylcarbamothioyl)benzamide (1.5 g, 4.0 mmol) and sodium ethoxide (0.54 g, 8.0 mmol) in 1-methyl-2-pyrrolidinone (10 mL) was heated to 120° C. for 8 hours. After cooling the reaction mixture to room temperature the mixture was poured into water. The resulting solid was collected by filtration, then washed sequentially with water and diethyl ether. The filter cake was dried to give N-(5-bromothiazolo[5,4-b]pyridin-2-yl)benzamide (1.02 g, 76%). ¹H NMR (400 MHz, d₆-DMSO): 13.2 (br s, 1H), 8.16-8.10 (m, 3H), 7.72 (d, 1H), 7.70 (t, 1H), 7.59 (t, 2H). MS (EI) for C₁₃H₈BrN₃OS: 336 (MH⁺).

### Reagent Preparation 14

STEP 1: To a solution of 2-amino-5-bromopyridine (5.0 g, 29 mmol) in dioxane (60 mL) was added ethoxycarbonylisothiocyanate (3.4 mL, 29 mmol) in a dropwise manner and the mixture was allowed to stir for 18 h at room temperature. The mixture was then concentrated and the residue triturated with 10% ethyl acetate in hexanes. The solid was collected by filtration and dried to afford ethyl {[(5-bromopyridin-2-yl)amino]carbonothioyl}carbamate (6.2 g, 69%) as a colorless solid. MS (EI) for C₉H₁₀BrN₃O₂S: 305 (MH⁺).

STEP 2: {[(5-Bromopyridin-2-yl)amino]carbonothioyl}carbamate was converted to 6-bromo-[1,2,4]triazolo[1,5-a]pyridin-2-amine according to methods in the literature, see 1) Monatshefte fuer Chemie, 1983, 114(6-7), 789-98 and 2) Synthesis, 2003, 11, 1649-1652. Thus, a mixture of hydroxylamine hydrochloride (375 mg, 5.4 mmol) and DIPEA (560 uL, 3.2 mmol) in 1:1 methanol:ethanol (8 mL) was stirred for 10 minutes at room temperature followed by addition of {[(5-bromopyridin-2-yl)amino]carbonothioyl}carbamate (500 mg, 1.62 mmol) and the resulting suspension was stirred for 2 h at room temperature then brought to 60° C. for an additional 2 h. The resulting solution was then cooled to room temperature and concentrated. The residue was then partitioned with ethyl acetate and saturated aqueous sodium bicarbonate. The organic solution was washed with brine, dried over anhydrous sodium sulfate then filtered and concentrated to give 6-bromo-[1,2,4]triazolo[1,5-a]pyridin-2-amine (340 mg, 98% yield) as a colorless crystalline solid. MS (EI) for C₆H₅BrN₄: 214 (MH⁺).

STEP 3: A solution of 6-bromo-[1,2,4]triazolo[1,5-a]pyridin-2-amine (340 mg, 1.6 mmol), di-tert-butyl dicarbonate (370 mg, 1.6 mmol) and catalytic DMAP was stirred at 35° C. in THF (5 mL) for 18 h. An additional equivalent of di-tert-butyl dicarbonate was then added and stirring was continued for 48 h. The solution was then partitioned with ethyl acetate and water. The organic phase was washed with brine, dried over anhydrous sodium sulfate then filtered and concentrated. The residue was taken into dichloromethane and insoluble starting material was removed by filtration. The filtrate was concentrated and purified by silica gel chromatography to afford bis-(1,1-dimethylethyl) (6-bromo[1,2,4]triazolo[1,5-a]pyridine-2-yl)imidodicarbonate (284 mg, 43% yield) as an off white solid. ¹H NMR (400 MHz, d₆-DMSO): 9.45 (s, 1H), 7.91 (d, 1H), 7.86 (d, 1H), 1.41 (s, 18H).

Using analogous synthetic techniques and substituting with alternative starting reagents bis(1,1-dimethylethyl) (5-bromo-4-methyl-1,3-thiazol-2-yl)imidodicarbonate was prepared, according to the method of reagent prepartion 14 using 5-bromo-4-methylthiazol-2-amine in step 3 and conducting the protection step at reflux temperature. ¹H NMR (400 MHz, CDCl₃): 2.30 (s, 3H), 1.53 (s, 18H).

### Reagent Preparation 15

### 6-bromo-1-trityl-1H-imidazo[4,5-b]pyridine and 6-bromo-3-trityl-3H-imidazo[4,5-b]pyridine

STEP 1: A suspension of 2,3-diamino-5-bromopyridine (3.0 g, 16.00 mmol) in formic acid (30 mL) was heated to reflux for 3 hours. After cooling the reaction mixture to room temperature it was concentrated and the residue was taken into 50% ethyl acetate in toluene (100 mL) then concentrated and the process repeated once more to remove excess formic acid. The resulting solid was triturated with ethyl acetate and the solid residue collected by filtration to give 6-bromo-1H-imidazo[4,5-b]pyridine (3.7 g, 95%). GCMS (EI) for C₆H₄BrN₃: 198 (M⁺).

STEP 2: To a solution of 6-bromo-1H-imidazo[4,5-b]pyridine (2.7 g, 11.0 mmol) in dimethylformamide (30 mL) at 0° C. was added 60% sodium hydride in mineral oil (0.53 g, 13.2 mmol) and the reaction mixture was stirred for 30 minutes, followed by the addition of a solution of triphenylmethyl chloride (3.2 g, 11.55 mmol) in dimethylformamide (5 mL). The reaction mixture was stirred at room temperature for 24 hours then quenched by the careful addition of water then partitioned with ethyl acetate (250 mL). The organic phase was washed with 10% aqueous citric acid (2×, 100 mL), brine (100 mL), saturated sodium bicarbonate (100 mL), brine (100 mL) then dried over anhydrous sodium sulfate, filtered and concentrated. Silica gel chromatography (hexane ethyl acetate 9:1 to 4:1) provided 6-bromo-3-trityl-3H-imidazo[4,5-b]pyridine (1.8 g, 37%). ¹H NMR (400 MHz, CDCl₃): 8.18 (d, 1H), 8.14 (d, 1H), 8.02 (s, 1H), 7.36-7.28 (m, 10H), 7.18-7.14 (m, 5H) and 6-bromo-1-trityl-1H-imidazo[4,5-b]pyridine (2.9 g, 60%) ¹H NMR (400 MHz, CDCl₃): 8.50 (d, 1H), 8.14 (s, 1H), 7.38-7.34 (m, 10H), 7.16-7.12 (m, 5H), 6.84 (d, 1H).

### Reagent Preparation 16

### N-(7-Bromo[1,2,4]triazolo[1,5-a]pyridin-2-yl)acetamide

STEP 1: To a solution of 7-Bromo-[1,2,4]triazolo[1,5-a]pyridin-2-ylamine (prepared using the procedure in WO2006038116) (0.150 g, 0.704 mmol), diisopropylethylamine (0.363 g, 2.81 mmol), catalytic DMAP (0.09 g, 0.07 mmol) in anhydrous THF (4 mL) was added acetic anhydride (0.216 g, 2.11 mmol). The reaction mixture was stirred at 50° C. for 22 h under N₂ (g). After cooling to room temperature the mixture was concentrated, diluted with ethyl acetate (50 mL), washed with saturated sodium bicarbonate (40 mL), brine (40 mL), and dried over anhydrous sodium sulfate. Filtration and concentration followed by column chromatography of the residue on silica (95:5 dicholormethane/methanol) afforded N-(7-bromo-[1,2,4]triazolo[1,5-c]pyridin-2-yl)acetamide (0.170 g, 95% yield) as a brown oil. MS (EI) for C₈H₇BrN₄O: 256 (MH⁺).

### Reagent Preparation 17

### 1-(4-chloro-6,6-dimethyl-5,6,7,8-tetrahydroquinazolin-2-yl)-N,N-dimethylmethanamine

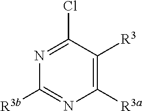

Step 1: To a solution of methyl 5,5-dimethyl-2-oxocyclohexanecarboxylate (6.0 g, 33 mmol) and 2-chloroacetimidamide hydrochloride (4.6 g, 36 mmol) in methanol (30 mL) was added sodium methoxide (4.4 M in MeOH, 9.0 mL, 40 mmol). The reaction mixture was stirred at ambient temperature for three hours and then concentrated. The resulting residue was partitioned between ethyl acetate and aqueous sodium bicarbonate. The organic layer was washed with brine, dried over magnesium sulfate and concentrated. Purification by silica gel chromatography provided 2-(chloromethyl)-6,6-dimethyl-5,6,7,8-tetrahydroquinazolin-4-ol (4.2 g, 57% yield) as a white solid. MS (ES) for C₁₁H₁₅ClN₂O: 227 (MH⁺).

Step 2: To a solution of 2-(chloromethyl)-6,6-dimethyl-5,6,7,8-tetrahydroquinazolin-4-ol (2.5 g, 11 mmol) in THF (10 mL) was added dimethyl amine (2M in THF, 16.5 mL, 33 mmol). The reaction mixture was heated (60° C.) for two hours and then partitioned between ethyl acetate and sodium bicarbonate. The organic layer was washed with brine, dried over magnesium sulfate, filtered and concentrated to provide 2-((dimethylamino)methyl)-6,6-dimethyl-5,6,7,8-tetrahydroquinazolin-4-ol, which was used in step 3 without further purification. MS (ES) for C₁₃H₂₁N₃O: 236 (MH⁺).

Step 3: To a solution of the final residue from step 2 in CHCl₃ (10 mL) was added POCl₃ (10 mL). The reaction mixture was heated (90° C.) for two hours and concentrated. This residue was partitioned between dichloromethane and aqueous sodium bicarbonate and the resulting organic layer was washed with brine, dried over magnesium sulfate, filtered and concentrated in vacuo. Purification by silica gel chromatography (5-10% concentrated aqueous ammonia in methanol) in chloroform provided 1-(4-chloro-6,6-dimethyl-5,6,7,8-tetrahydroquinazolin-2-yl)-N,N-dimethylmethanamine (1.3 g, 48% yield). ¹H NMR (400 MHz, CD₃OD) δ 4.52 (s, 2H), 3.02 (s, 6H), 2.98 (t, 2H), 2.61 (s, 2H), 1.71 (t, 2H), 1.06 (s, 6H); MS (ES) for C₁₃H₂₀ClN₃: 254 (MH⁺)

Using analogous synthetic techniques and substituting with alternative starting reagents the following compounds of the invention were prepared. Alternative starting materials were obtained commercially unless otherwise indicated.

(S)-4-chloro-2-((3-fluoropyrrolidin-1-yl)methyl)-6,6-dimethyl-5,6,7,8-tetrahydroquinazoline. Synthesized according to the method of reagent preparation 17 using (S)-3-fluoropyrrolidine in step 2. MS (ES) for C₁₅H₂₁ClFN₃: 298 (MH⁺).

(R)-4-chloro-2-((3-fluoropyrrolidin-1-yl)methyl)-6,6-dimethyl-5,6,7,8-tetrahydroquinazoline. Synthesized according to the method of reagent preparation 17 using (R)-3-fluoropyrrolidine in step 2. MS (ES) for C₁₅H₂₁ClFN₃: 298 (MH⁺).

4-chloro-2-((3,3-difluoropyrrolidin-1-yl)methyl)-6,6-dimethyl-5,6,7,8-tetrahydroquinazoline. Synthesized according to the method of reagent preparation 17 using 3,3-difluoropyrrolidine in step 2. MS (ES) for C₁₅H₂₀ClF₂N₃: 316 (MH⁺).

N-((4-chloro-6,6-dimethyl-5,6,7,8-tetrahydroquinazolin-2-yl)methyl)-N-methylethanamine. Synthesized according to the method of reagent preparation 17 using N-methylethanamine in step 2. MS (ES) for C₁₄H₂₂ClN₃: 268 (MH⁺).

4-chloro-6,6-dimethyl-2-(piperidin-1-ylmethyl)-5,6,7,8-tetrahydroquinazoline. Synthesized according to the method of reagent preparation 17 using piperidine in step 2. MS (ES) for C₁₆H₂₄ClN₃: 294 (MH⁺).

N-((4-chloro-6,6-dimethyl-5,6,7,8-tetrahydroquinazolin-2-yl)methyl)-N-methylpropan-2-amine. Synthesized according to the method of reagent preparation 17 using N-methylpropan-2-amine in step 2. MS (ES) for C₁₅H₂₄ClN₃: 282 (MH⁺).

N-((4-chloro-6,6-dimethyl-5,6,7,8-tetrahydroquinazolin-2-yl)methyl)-N-methylcyclopropanamine. Synthesized according to the method of reagent preparation 17 using N-methylcyclopropanamine in step 2. MS (ES) for C₁₅H₂₂ClN₃: 280 (MH⁺).

Benzyl (4-chloro-6,6-dimethyl-5,6,7,8-tetrahydroquinazolin-2-yl)methyl(isopropyl)carbamate. Synthesized according to the method of reagent preparation 17 using propane-2-amine in step 2 followed by Cbz protection. MS (ES) for C₂₂H₂₈ClN₃O₂: 402 (MH⁺).

4-chloro-6,6-dimethyl-2-(pyrrolidin-1-ylmethyl)-5,6,7,8-tetrahydroquinazoline. Synthesized according to the method of reagent preparation 17 using pyrrolidine in step 2. MS (ES) for C₁₅H₂₂ClN₃: 280 (MH⁺).

(S)-1-(4-chloro-7-ethyl-5,6,7,8-tetrahydroquinazolin-2-yl)-N,N-dimethylmethanamine. Synthesized according to the method of reagent preparation 17 using (S)-methyl 4-ethyl-2-hydroxycyclohex-1-enecarboxylate in step 1. MS (ES) for C₁₃H₂₀ClN₃: 254 (MH⁺).

{4-chloro-5-[(4-fluorophenyl)methyl]-6-methylpyrimidin-2-yl}methyl acetate. Synthesized according to the method of reagent preparation 17 using 2-(chloromethyl)-5-[(4-fluorophenyl)methyl]-6-methylpyrimidin-4-ol and sodium acetate in acetic acid in step 2. MS (ES) for C₁₅H₁₄ClFN₂O₂: 309 (MH⁺).

4-chloro-2-(methoxymethyl)-6,6-dimethyl-5,6,7,8-tetrahydroquinazoline. Synthesized according to the method of reagent preparation 17 using sodium methoxide in step 2. MS (ES) for C₁₂H₁₇ClN₂O: 241 (MH⁺).

Benzyl (4-chloro-6,6-dimethyl-5,6,7,8-tetrahydroquinazolin-2-yl)methyl(ethyl)-carbamate. Prepared according to the method of reagent preparation 17 by using ethylamine in step 2 followed by Cbz protection. MS (EI) for C₂₁H₂₆ClN₃O₂: 388 (MH⁺).

Benzyl (4-chloro-6,6-dimethyl-5,6,7,8-tetrahydroquinazolin-2-yl)methyl(2-fluoroethyl)carbamate. Prepared according to the method of reagent preparation 17 by using fluoroethylamine in step 2 followed by Cbz protection. MS (EI) for C₂₁H₂₅ClFN₃O₂: 406 (MH⁺).

N-[(4-chloro-6,6-dimethyl-5,6,7,8-tetrahydroquinazolin-2-yl)methyl]cyclopropanamine. Prepared according to the method of reagent preparation 17 by using cyclopropylamine in step 2. MS (EI) for C₁₄H₂₀ClN₃: 266 (MH⁺).

Benzyl (4-chloro-6,6-dimethyl-5,6,7,8-tetrahydroquinazolin-2-yl)methyl(cyclobutyl)carbamate. Prepared according to the method of reagent preparation 17 by using cyclobutylamine in step 2 followed by Cbz protection. MS (EI) for C₂₃H₂₈ClN₃O₂: 414 (MH⁺).

1-(4-Chloro-5-(cyclopropylmethyl)-6-methylpyrimidin-2-yl)-N,N-dimethylmethanamine. Prepared according to the method of reagent preparation 17 by using methyl 2-(cyclopropylmethyl)-3-oxobutanoate (reagent preparation 8) in step 1. MS (EI) for C₁₂H₁₈ClN₃: 240 (MH⁺).

N-((4-chloro-6,6-dimethyl-5,6,7,8-tetrahydroquinazolin-2-yl)methyl)-N-ethylethanamine. Prepared according to the method of reagent preparation 17 by using diethylamine in step 2. MS (EI) for C₁₅H₂₄ClN₃: 282 (MH⁺).

4-((4-Chloro-6,6-dimethyl-5,6,7,8-tetrahydroquinazolin-2-yl)methyl)morpholine. Prepared according to the method of reagent preparation 17 by using morpholine in step 2. MS (EI) for C₁₅H₂₂ClN₃O: 296 (MH⁺).

N-((4-chloro-6,6-dimethyl-5,6,7,8-tetrahydroquinazolin-2-yl)methyl)-N-ethylpropan-2-amine. Prepared according to the method of reagent preparation 17 by using ethylisopropylamine in step 2. MS (EI) for C₁₆H₂₆ClN₃: 296 (MH⁺).

N-((4-chloro-6,6-dimethyl-5,6,7,8-tetrahydroquinazolin-2-yl)methyl)-2-methylpropan-2-amine. Prepared according to the method of reagent preparation 17 by using tert-butylamine in step 2. MS (EI) for C₁₅H₂₄ClN₃: 282 (MH⁺).

N-((4-chloro-6,6-dimethyl-5,6,7,8-tetrahydroquinazolin-2-yl)methyl)-2-methylpropan-1-amine. Prepared according to the method of reagent preparation 17 by using iso-butylamine in step 2. MS (EI) for C₁₅H₂₄ClN₃: 282 (MH⁺).

Benzyl (4-chloro-6,6-dimethyl-5,6,7,8-tetrahydroquinazolin-2-yl)methyl(2,2-difluoroethyl)carbamate. Prepared according to the method of reagent preparation 17 by using 2,2-difluoroethylamine in step 2 followed by Cbz protection. MS (EI) for C₂₁H₂₄ClF₂N₃O₂: 424 (MH⁺).

N-((4-chloro-6,6-dimethyl-5,6,7,8-tetrahydroquinazolin-2-yl)methyl)-2,2,2-trifluoroethanamine. Prepared according to the method of reagent preparation 17 by using 2,2,2-trifluoroethylamine in step 2. MS (EI) for C₁₃H₁₇ClF₃N₃: 308 (MH⁺).

N-((4-chloro-6,6-dimethyl-5,6,7,8-tetrahydroquinazolin-2-yl)methyl)-1-cyclopropylethanamine. Prepared according to the method of reagent preparation 17 by using 1-cyclopropylethanamine in step 2. MS (EI) for C₁₆H₂₄ClN₃: 294 (MH⁺).

(4-Chloro-6,6-dimethyl-5,6,7,8-tetrahydroquinazolin-2-yl)methyl acetate. Prepared according to the method of reagent preparation 17 by using potassium acetate in step 2. MS (EI) for C₁₃H₁₇ClN₂O₂: 269 (MH⁺).

Benzyl (4-chloro-6,6-dimethyl-5,6,7,8-tetrahydroquinazolin-2-yl)methyl(cyclopentyl)carbamate. Prepared according to the method of reagent preparation 17 by using cyclopentylamine in step 2 followed by Cbz protection. MS (EI) for C₂₄H₃₀ClN₃O₂: 428 (MH⁺).

Ethyl 2-((4-chloro-6,6-dimethyl-5,6,7,8-tetrahydroquinazolin-2-yl)methylamino)propanoate. Prepared according to the method of reagent preparation 17 by using alanine ethyl ester in step 2. MS (EI) for C₁₆H₂₄ClN₃O₂: 326 (MH⁺).

1-(4-Chloro-5,6-dimethylpyrimidin-2-yl)-N,N-dimethylmethanamine. Prepared according to the method of reagent preparation 17 by using methyl 2-methyl-3-oxobutanoate in step 1 in step 2. MS (EI) for C₉H₁₄ClN₃: 200 (MH⁺).

1-(4-chloro-5-(4-fluorobenzyl)-6-methylpyrimidin-2-yl)-N,N-dimethylmethanamine. Synthesized according to the method of reagent preparation 17 using methyl 2-(4-fluorobenzyl)-3-oxobutanoate in step 1. ¹H NMR (400 MHz, CDCl₃): 7.08-7.05 (m, 2H), 7.00-6.96 (m, 2H), 4.14 (s, 2H), 3.68 (s, 2H), 2.51 (s, 3H), 2.38 (s, 6H).

1-(4-chloro-5-isopropyl-6-methylpyrimidin-2-yl)-N,N-dimethylmethanamine. Synthesized according to the method of reagent preparation 17 using methyl 2-acetyl-3-methylbutanoate in step 1. MS (EI) for C₁₁H₁₈N₃Cl: 228, 230 (MH⁺, Cl isotope pattern).

(S)-benzyl sec-butyl((4-chloro-6,6-dimethyl-5,6,7,8-tetrahydroquinazolin-2-yl)methyl)carbamate Synthesized according to the method of reagent preparation 17 using (S)-butan-2-amine in step 2 followed Cbz-protection prior to step 3. MS (ES) for C₂₃H₃₀ClN₃O₂: 416 (MH⁺).

(R)-benzyl sec-butyl((4-chloro-6,6-dimethyl-5,6,7,8-tetrahydroquinazolin-2-yl)methyl)carbamate Synthesized according to the method of reagent preparation 17 using (R)-butan-2-amine in step 2 followed Cbz-protection prior to step 3. MS (ES) for C₂₃H₃₀ClN₃O₂: 416 (MH⁺).

1-(4-chloro-6-ethyl-5-methylpyrimidin-2-yl)-N,N-dimethylmethanamine Synthesized according to the method of reagent preparation 17 using methyl 2-methyl-3-oxopentanoate in step 1. MS (ES) for C₁₀H₁₆ClN₃: 214 (MH⁺).

1-(4-chloro-5-isopropylpyrimidin-2-yl)-N,N-dimethylmethanamine Synthesized according to the method of reagent preparation 17 using methyl 2-methyl-3-oxopentanoate (Elaridi et al. Tetrahedron: Asymmetry 2005, 16(7), 1309-1319) in step 1.

N-((4-chloro-6,6-dimethyl-5,6,7,8-tetrahydroquinazolin-2-yl)methyl)-N-methyl-2-nitrobenzenesulfonamide Synthesized according to the method of reagent preparation 17 using methylamine in step 2 followed by protection as the 2-nitrobenzenesulfonamide prior to step 3. ¹H NMR (400 MHz, CDCl₃) δ 8.18-8.13 (m, 1H), 7.71-7.62 (m, 2H), 7.61-7.57 (m, 1H), 4.69 (s, 2H), 3.08 (d, 3H), 2.73 (t, 2H), 2.47 (s, 2H), 1.60 (t, 2H), 1.01 (s, 6H); MS (ES) for C₁₈H₂₁ClN₄O₄S: 425 (MH⁺).

N-((4-chloro-6,6-dimethyl-5,6,7,8-tetrahydroquinazolin-2-yl)methyl)methanesulfonamide Synthesized according to the method of reagent preparation 17 using ammonia in step 2 followed by mesylation prior to step 3. ¹H NMR (400 MHz, CDCl₃) δ 4.49 (d, 2H), 3.01 (s, 3H), 2.90 (t, 2H), 2.54 (s, 2H), 1.67 (t, 2H), 1.05 (s, 6H); MS (ES) for C₁₂H₁₈ClN₃O₂S: 304 (MH⁺).

1-(4-chloro-5-ethyl-6-methylpyrimidin-2-yl)-N,N-dimethylmethanamine. Synthesized according to the method of reagent preparation 17 using ethyl 2-ethyl-3-oxobutanoate in step 1. ¹H NMR (400 MHz, CDCl₃) δ 3.64 (s, 2H), 2.78 (q, 2H), 2.58 (s, 3H), 2.36 (s, 6H), 1.19 (t, 3H); MS (ES) for C₁₀H₁₆ClN₃: 214 (MH⁺).

4-chloro-6,6-dimethyl-2-({[2-(methyloxy)ethyl]oxy}methyl)-5,6,7,8-tetrahydroquinazoline. Synthesized according to the method of reagent preparation 17 using sodium hydride and 2-methoxyethanol in N,N-dimethylformamide) in step 2. MS (ES) for C₁₄H₂₁ClN₂O₂: 285 (MH⁺).

N-[(4-chloro-6,6-dimethyl-5,6,7,8-tetrahydroquinazolin-2-yl)methyl]-2-(methyloxy)ethanamine. Synthesized according to the method of reagent preparation 17 using 2-methoxyethanamine in step 2. MS (ES) for C₁₄H₂₂ClN₃O: 284 (MH⁺).

N-((4-chloro-5-(4-fluorobenzyl)-6-methylpyrimidin-2-yl)methyl)cyclopropanamine. Prepared according to the method of reagent preparation 17 by using methyl 2-(4-fluorobenzyl)-3-oxobutanoate in step 1 and cyclopropylamine in step 2. MS (EI) for C₁₆H₁₇ClFN₃: 306 (MH⁺).

1-(4-chloro-7-methoxy-6,6-dimethyl-5,6,7,8-tetrahydroquinazolin-2-yl)-N,N-dimethylmethanamine. Prepared according to the method of reagent preparation 17 using methyl 5,5-dimethyl-2-oxocyclohex-3-enecarboxylate (Can. J. Chem., 1981, 59, 601-608) in step 1. MS (ES) for C₁₄H₂₂ClN₃O: 284 (MH⁺).

### Reagent Preparation 18

### Phenylmethyl (2R)-2-(4-chloro-6,6-dimethyl-5,6,7,8-tetrahydroquinazolin-2-yl)pyrrolidine-1-carboxylate

STEP 1: To sodium methoxide (30 wt % in methanol, 8 mg, 0.05 mmol) was added a solution of (R)-benzyl 2-cyanopyrrolidine-1-carboxylate (189 mg, 0.82 mmol) in methanol (1 mL) at room temperature and the reaction mixture was stirred for one hour. Ammoniun chloride (44 mg, 0.82 mmol) was introduced and the stirring was continued for an additional two hours, followed by the addition of methyl 5,5-dimethyl-2-oxocyclohexanecarboxylate (100 mg, 0.54 mmol) and sodium methoxide (30 wt % in methanol, 293 mg, 1.63 mmol). The stirring was continued for two more hours. The reaction mixture was quenched with water (10 mL), neutralized with 1 N hydrohloric acid and extracted with ethyl acetate (3×10 mL). The combined extract was washed with water (20 mL) and brine, dried over sodium sulfate, filtered, concentrated and purified by gradient flash chromatography (25% to 95% ethyl acetate in hexane) to give phenylmethyl (2R)-2-(4-hydroxy-6,6-dimethyl-5,6,7,8-tetrahydroquinazolin-2-yl)pyrrolidine-1-carboxylate (186 mg, 90%). MS (EI) for C₂₂H₂₇N₃O₃: 381 (MH⁺).

STEP 2: A mixture phenylmethyl (2R)-2-(4-hydroxy-6,6-dimethyl-5,6,7,8-tetrahydroquinazolin-2-yl)pyrrolidine-1-carboxylate (150 mg, 0.39 mmol) and phosphorous oxychloride (1 mL) in chloroform (3 mL) was stirred at 80° C. for one hour. After cooling to room temperature the reaction mixture was concentrated and the residue was partitioned between saturated sodium bicarbonate (20 mL) and ethyl acetate (20 mL). The mixture was stirred for 15 minutes and pH was maintained above 7 by the addition of solid sodium bicarbonate. The organic layer was separated and washed with water (10 mL) and brine, dried over sodium sulfate, filtered and concentrated to give phenylmethyl (2R)-2-(4-chloro-6,6-dimethyl-5,6,7,8-tetrahydroquinazolin-2-yl)pyrrolidine-1-carboxylate (117 mg, 74%). MS (EI) for C₂₂H₂₆ClN₃O₂: 400 (MH⁺).

Using analogous synthetic techniques and substituting with alternative starting materials in step 1 the following reagents of the invention were prepared. Alternative starting materials were obtained commercially unless otherwise indicated.

Phenylmethyl (2S)-2-(4-chloro-6,6-dimethyl-5,6,7,8-tetrahydroquinazolin-2-yl)pyrrolidine-1-carboxylate. Prepared according to the method of reagent preparation 18 by using (S)-benzyl 2-cyanopyrrolidine-1-carboxylate in step 1 (118 mg, 75%). MS (EI) for C₂₂H₂₆ClN₃O₂: 400 (MH⁺).

Phenylmethyl 2-(4-chloro-6,6-dimethyl-5,6,7,8-tetrahydroquinazolin-2-yl)pyrrolidine-1-carboxylate. Prepared according to the method of reagent preparation 18 by using (R,S)-benzyl 2-cyanopyrrolidine-1-carboxylate in step 1 (118 mg, 75%). MS (EI) for C₂₂H₂₆ClN₃O₂: 400 (MH⁺).

### Reagent Preparation 19

### Phenylmethyl{[6-bromo-3-({[2-(trimethylsilyl)ethyl]oxy}methyl)-3H-imidazo[4,5-b]pyridin-2-yl]methyl}methylcarbamate

STEP 1: To a mixture of 2-[(benzyloxycarbonyl)(methyl)amino]acetic acid (0.42 g, 1.88 mmol), O-(7-azabenzotriazol-1-yl)-N,N,N′,N′-tetramethyluronium hexafluorophosphate (0.75 g, 1.97 mmol) in N,N-dimethylformamide (3.0 mL), N,N-diisopropylethylamine (0.72 mL, 4.12 mmol) was added and the reaction mixture was stirred for 30 minutes at room temperature, followed by the addition of 5-bromo-2,3-diaminopyridine (0.35 g, 1.86 mmol), then stirred for 16 hours. It was diluted with ethyl acetate (50 mL), washed with aqueous lithium chloride (2×20 mL) and brine, dried over sodium sulfate, filtered and concentrated. Gradient flash chromatography (35% to 85% ethyl acetate in hexane) provided phenylmethyl{2-[(2-amino-5-bromopyridin-3-yl)amino]-2-oxoethyl}methylcarbamate (0.70 g, 96%). MS (EI) for C₁₆H₁₇BrN₄O₃: 394 (MH⁺).

STEP 2: A solution of phenylmethyl{2-[(2-amino-5-bromopyridin-3-yl)amino]-2-oxoethyl}methylcarbamate (0.30 g, 0.76 mmol) in acetic acid (7.5 mL) was heated in a microwave apparatus (250 W) for 30 min. at 120° C. After cooling it to room temperature the reaction mixture was concentrated and the pH was adjusted to 8 by the addition of saturated aqueous sodium bicarbonate. The precipitating solid was collected by filtration, washed with water and dried in vacuo to give phenylmethyl[(6-bromo-1H-imidazo[4,5-b]pyridin-2-yl)methyl]methylcarbamate (0.22 g, 76%). MS (EI) for C₁₆H₁₅BrN₄O₂: 376 (MH⁺).

STEP 3: To a solution of phenylmethyl[(6-bromo-1H-imidazo[4,5-b]pyridin-2-yl)methyl]methylcarbamate (0.22 g, 0.59 mmol) in N,N-dimethylformamide (3.0 mL) was added 60% sodium hydride in mineral oil (56 mg, 1.48 mmol) and the reaction mixture was stirred for 30 minutes at room temperature, followed by the addition of 2-(trimethylsilyl)ethoxymethyl chloride (0.11 mL, 0.62 mmol). The reaction mixture was stirred at room temperature for 16 hours then it was quenched by the careful addition of saturated aqueous ammonium chloride and partitioned with ethyl acetate (20 mL) and water (20 mL). The organic layer was separated and washed with 10% aqueous citric acid (2×20 mL) and brine (20 mL), dried over sodium sulfate, filtered and concentrated. Gradient flash chromatography (15% to 35% ethyl acetate in hexane) gave phenylmethyl{[6-bromo-3-({[2-(trimethylsilyl)ethyl]oxy}methyl)-3H-imidazo[4,5-b]pyridin-2-yl]methyl}methylcarbamate (0.28 g, 93%). MS (EI) for C₂₂H₂₉BrN₄O₃Si: 506 (MH⁺).

Using analogous synthetic techniques and substituting with alternative starting materials and reagents in step 1 or step 2 and step 3 the following reagents of the invention were prepared. Alternative starting materials were obtained commercially unless otherwise indicated.

Phenylmethyl{(1R)-1-[6-bromo-3-({[2-(trimethylsilyl)ethyl]oxy}methyl)-3H-imidazo[4,5-b]pyridin-2-yl]ethyl}({[2-(trimethylsilyl)ethyl]oxy}methyl)carbamate. Synthesized according to the method of reagent preparation 19 by using 5-bromo-2,3-diaminopyridine and N-(benzyloxycarbonyl)-D-alanine in step 1 and 2-(trimethylsilyl)ethoxymethyl chloride in step 3. MS (EI) for C₂₈H₄₃BrN₃₄O₄Si₂: 636 (MH⁺).

Phenylmethyl{(1S)-1-[6-bromo-3-({[2-(trimethylsilyl)ethyl]oxy}methyl)-3H-imidazo[4,5-b]pyridin-2-yl)ethyl}({[2-(trimethylsilyl)ethyl]oxy}methyl)carbamate. Synthesized according to the method of reagent preparation 19 by using 5-bromo-2,3-diaminopyridine and N-(benzyloxycarbonyl)-L-alanine in step 1 and 2-(trimethylsilyl)ethoxymethyl chloride in step 3. MS (EI) for C₂₈H₄₃BrN₃₄O₄Si₂: 636 (MH⁺).

7-Bromo-2-methyl-3-({[2-(methyloxy)ethyl]oxy}methyl)-3H-imidazo[4,5-c]pyridine and 7-bromo-2-methyl-1-({[2-(methyloxy)ethyl]oxy}methyl)-1H-imidazo[4,5-c]pyridine. Synthesized according to the method of reagent preparation 19 by using 5-bromopyridine-3,4-diamine and triethyl orthoacetate in step 2 and methoxyethoxymethyl chloride in step 3. ¹H NMR (400 MHz, CDCl₃): 8.83 (s, 2H), 8.44 (s, 2H), 5.88 (s, 2H), 5.66 (s, 2H), 3.36 (s, 3H), 3.37 (s, 3H), 2.98 (s, 4H), 2.91 (s, 4H), 2.73 (s, 3H), 2.75 (s, 3H); MS (EI) for C₁₁H₁₄BrN₃O₂: 301 (MH⁺).

1-(6-Bromo-3H-imidazo[4,5-b]pyridin-2-yl)ethanol. Synthesized according to the method of reagent preparation 19 by using D,L-lactic acid in step 1. MS (EI) for C₈H₈BrN₃O: 241 (MH−).

Tert-butyl 6-bromo-2-(difluoromethyl)-1H-benzo[d]imidazole-1-carboxylate. Synthesized according to the method of reagent preparation 19 using 4-bromobenzene-1,2-diamine and difluoroacetic acid in step 1 and BOC protection with di-tert-butyl dicarbonate in step 3. MS (EI) for 6-bromo-2-(difluoromethyl)-1H-benzo[d]imidazole (step 2) C₅H₅BrF₂N₂: 247, 249 (MH⁺, Br isotope pattern).

1,1-Dimethylethyl 6-bromo-2,4-dimethyl-1H-benzimidazole-1-carboxylate. Synthesized according to the method of reagent preparation 19 using 5-bromo-3-methylbenzene-1,2-diamine and acetylation using acetyl chloride in tetrahydrofuran in step 1 the BOC protection with di-tert-butyl dicarbonate in step 3. MS (EI) for C₁₄H₁₇BrN₂O₂: 267, 269 (M-Boc, Br isotope pattern).

1,1-Dimethylethyl 5-bromo-6-fluoro-2-methyl-1H-benzimidazole-1-carboxylate. Synthesized according to the method of reagent preparation 19 using 4-bromo-5-fluorobenzene-1,2-diamine and triethyl orthoacetate in step 2 and BOC protection with di-tert-butyl dicarbonate in step 3. MS (EI) for C₁₃H₁₄BrFN₂O₂: 271, 273 (M-Boc, Br isotope pattern).

2-Methylpropyl 5-bromo-4-fluoro-2-methyl-1H-benzimidazole-1-carboxylate. Synthesized according to the method of reagent preparation 19 using 5 4-bromo-3-fluorobenzene-1,2-diamine and acetylation with acetic anhydride in tetrahydrofurane in step 1 then treatment with isobutyl chloroformate in step 3. MS (EI) for C₁₃H₁₄BrN₂O₂: 328, 330 (MH⁺, Br isotope pattern).

6-Bromo-2-ethyl-3-({[2-(trimethylsilyl)ethyl]oxy}methyl)-3H-imidazo[4,5-b]pyridine. Synthesized according to the method of reagent preparation 19 by using 5-bromo-2,3-diaminopyridine and trimethyl orthopropionate in step 2 and 2-(trimethylsilyl)ethoxymethyl chloride in step 3. MS (EI) for C₁₄H₂₂BrN₃OSi: 357 (MH⁺).

2-Methylpropyl 6-bromo-2-cyclopropyl-3H-imidazo[4,5-b]pyridine-3-carboxylate. Synthesized according to the method of reagent preparation 19 by using 5-bromo-2,3-diaminopyridine and acylation with cyclopropylcarbonyl chloride in step 1 and treatment with isobutyl chloroformate in step 3. MS (EI) for C₁₄H₁₆BrN₃O₂: 339 (MH⁺).

2-Methylpropyl 5-bromo-2-(fluoromethyl)-1H-benzimidazole-1-carboxylate. Synthesized according to the method of reagent preparation 19 using 4-bromobenzene-1,2-diamine and fluoroacetic acid in step 1 then treatment with isobutyl chloroformate in step 3. MS (EI) for C₁₃H₁₄BrFN₂O₂: 330 (MH⁺).

### Reagent Preparation 20

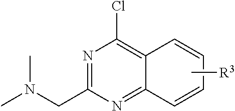

STEP 1: To a solution of 4-methoxyanthranilic acid (5.0 g, 30.0 mmol) in a mixture of 10% methanol in tetrahydrofuran (100 mL) was added dropwise (trimethylsilyl)diazomethane (2.0 M solution in diethyl ether, 18.0 mL, 36.0 mmol) at 0° C. The reaction mixture was stirred for 16 hours at room temperature then quenched by the addition of glacial acetic acid (0.1 mL). The reaction mixture was concentrated and the residue was partitioned between saturated sodium bicarbonate (50 mL) and ethyl acetate (250 mL). The organic layer was separated and washed with water (50 mL), saturated sodium bicarbonate (50 mL) and brine (50 mL), dried over sodium sulfate, filtered and concentrated to give methyl 2-amino-4-methoxybenzoate as an oil (5.4 g, quantitative). MS (EI) for C₉H₁₁NO₃: 182 (MH⁺).

STEP 2: To a mixture of methyl 2-amino-4-methoxybenzoate (5.4 g, 30.0 mmol) and chloroacetonitrile (2.8 mL, 45.0 mmol) was added anhydrous hydrogen chloride (4M solution in 1,4-dioxane, 20.0 mL, 80 mmol) and the reaction mixture was stirred at 50° C. for 30 minutes. After cooling it to room temperature the resulting slurry was diluted with diethyl ether (100 mL) and the stirring was continued for an additional 30 minutes. The off-white precipitate was collected by filtration, washed with diethyl ether and dried in vacuo to provide 2-(chloromethyl)-7-(methyloxy)quinazolin-4-ol hydrochloride (7.5 g, 96%). MS (EI) for C₁₀H₉ClN₂O₂: 225 (MH⁺).

STEP 3: To a solution of dimethylamine (2M solution in tetrahydrofuran, 40.0 mL, 80.0 mmol) was added 2-(chloromethyl)-7-(methyloxy)quinazolin-4-ol hydrochloride (7.5 g, 29 mmol) and the reaction mixture was stirred for 90 minutes at 50° C. After cooling it to room temperature the reaction mixture was concentrated and the residue was partitioned between water (100 mL) and ethyl acetate (250 mL). The organic layer was separated and washed with water (100 mL), saturated sodium bicarbonate (100 mL) and brine (100 mL), dried over sodium sulfate, filtered and concentrated to give 2-[(dimethylamino)methyl]-7-(methyloxy)quinazolin-4-ol (6.6 g, 97%). MS (EI) for C₁₂H₁₅N₃O₂: 234 (MH⁺).

STEP 4: A solution of 2-[(dimethylamino)methyl]-7-(methyloxy)quinazolin-4-ol (6.6 g, 28.0 mmol) in a mixture of chloroform (15.0 mL) and phosphorous oxychloride (15.0 mL) was heated to reflux for 90 minutes. After cooling it to room temperature the reaction mixture was concentrated and the residue was partitioned between saturated sodium bicarbonate (100 mL) and ethyl acetate (400 mL) and the mixture was stirred for 30 minutes. The organic layer was separated and washed with saturated sodium bicarbonate (2×100 mL) and brine (200 mL), dried over sodium sulfate, filtered and concentrated. Purification by silica gel column chromatography using 15% methanol containing 0.5% triethylamine in ethyl acetate provided 1-[4-chloro-7-(methyloxy)quinazolin-2-yl]-N,N-dimethylmethanamine (7.0 g, quantitative). MS (EI) for C₁₂H₁₄ClN₃O: 252 (MH⁺).

Using analogous synthetic techniques and substituting with alternative starting materials in step 2 the following reagents of the invention were prepared. Alternative starting materials were obtained commercially unless otherwise indicated.

1-(4-chloro-6-fluoroquinazolin-2-yl)-N,N-dimethylmethanamine. Prepared according to the method of reagent preparation 20 by using methyl 2-amino-5-fluorobenzoate in step 2. MS (EI) for C₁₁H₁₁ClFN₃: 240 (MH⁺).

### Reagent Preparation 21

### 5-Bromo-1-methyl-1H-pyrrolo[2,3-b]pyridine

STEP 1: To a mixture of 5-bromo-1H-pyrrolo[2,3-b]pyridine (207 mg, 1.05 mmol), sodium hydride (29 mg, 1.21 mmol) in tetrahydrofuran (5 mL) was added iodomethane (164 mg, 1.15 mol) then stirred for 2 h at room temperature. The reaction mixture was carefully quenched with water then extracted with ethyl acetate (3×). The combined organic layers were dried over magnesium sulfate, filtered and concentrated under reduced pressure. The crude product was purified by silica gel chromatography to give 5-bromo-1-methyl-1H-pyrrolo[2,3-b]pyridine. MS (EI) for C₈H₇BrN₂: 209, 211 (MH⁺, Br pattern).

Using analogous synthetic techniques and substituting with alternative starting reagents in step 1 the following reagent was prepared. 5-bromo-1-ethyl-1H-pyrrolo[2,3-b]pyridine. Synthesized according to the method of reagent preparation 21 using iodoethane. MS (EI) for C₉H₉BrN₂: 223, 225 (MH⁺, Br pattern).

### Reagent Preparation 22

### (4-(4-Bromophenyl)-1H-imidazol-2-yl)methanol

STEP 1: To a solution of ethyl thiooxamate (10.0 g, 75 mmol) in dichloromethane (400 mL) was slowly added trimethyloxonium tetrafluoroborate (13.1 g, 89 mmol) at 0° C. After 10 min the ice bath was removed, and the reaction mixture was stirred overnight. The solvent was removed to afford ethyl 2-imino-2-(methylthio)acetate (12.0 g, 66.6%) as tetrafluoroborate salt which was used without further purification.

STEP 2: A mixture of 2-amino-4-bromoacetophenone hydrochloride (4.0 g, 16.0 mmol), sodium acetate (6.1 g, 90.0 mmol), acetic acid (4.6 mL, 80.0 mmol) and ethyl 2-imino-2-(methylthio)acetate (7.7 g, 32.0 mmol) in dioxane (40 mL) was stirred at 95° C. overnight. The reaction mixture was carefully neutralized with saturated NaHCO3 solution and extracted with ethyl acetate. The organic solution was dried over sodium sulfate and concentrated. Purification by silica gel column chromatography (ethyl acetate:hexanes 1:1) afforded ethyl 4-(4-bromophenyl)-1H-imidazole-2-carboxylate (3.53 g, 75.0%). MS (EI) for C₁₂H₁₁BrN₂O₂: 296 (MH⁺).

STEP 3: To a solution of ethyl 4-(4-bromophenyl)-1H-imidazole-2-carboxylate (1.30 g, 4.40 mmol) in THF (30 mL) was slowly added Red-Al (65 wt % in toluene, 2.0 mL, 6.16 mmol) at −25° C. The reaction mixture was stirred for 4 h at the same temperature then slowly warmed to 0° C. over 1 h and quenched with 20% sodium tartrate solution (30 mL). The reaction was extracted with ethyl acetate (70 mL) and the organic layer was left for 3 h at room temperature. A solid separated and was collected by filtration, washed with ethyl acetate and dried to afford (4-(4-bromophenyl)-1H-imidazol-2-yl)methanol (778 mg, 71.0%). MS (EI) for C₁₀H₉BrN₂O: 254.1 (MH⁺).

### Reagent Preparation 23

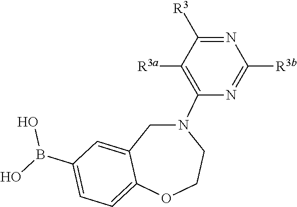

Step 1: To a slurry of 2,3,4,5-tetrahydro-1,4-benzoxazepin-7-ylboronic acid hydrochloride salt (5.7 g, 25 mmol) (example 8, step 1) and 4-chloro-6,6-dimethyl-5,6,7,8-tetrahydroquinazoline (reagent preparation 3) (3.0 g, 15 mmol) in dioxane (75 mL) and H₂O (75 mL) was added DIPEA (17 mL, 100 mmol) and the resulting mixture was heated (90° C.). After 72 hours the solution was concentrated and partitioned between 2M aqueous sodium hydroxide and ethyl ether. The aqueous layer was neutralized and extracted with chloroform. The organic layer was washed with brine, dried over magnesium sulfate, filtered and concentrated in vacuo. Trituration with ethyl ether provided [4-(6,6-dimethyl-5,6,7,8-tetrahydroquinazolin-4-yl)-2,3,4,5-tetrahydro-1,4-benzoxazepin-7-yl]boronic acid (4.2 g, 80% yield) as a white solid. ¹H NMR (400 MHz, d₆-DMSO) δ 8.68 (s, 1H), 7.77 (s, 1H), 7.64 (dd, 1H), 6.86 (dd, 1H), 5.04 (s, 2H), 4.46 (m, 2H), 4.18 (m, 2H), 2.80 (t, 2H), 2.52 (s, 2H), 1.58 (t, 2H), 0.86 (s, 6H); MS (ES) for C₁₉H₂₄BN₃O₃: 354 (MH⁺).

Using analogous synthetic techniques and substituting with alternative starting reagents in step 1 the following compounds of the invention were prepared. Alternative starting materials were obtained commercially unless otherwise indicated.

[4-(6,6,7-trimethyl-5,6-dihydroquinazolin-4-yl)-2,3,4,5-tetrahydro-1,4-benzoxazepin-7-yl]boronic acid. Synthesized according to the method of reagent preparation 23 using 4-chloro-6,6,7-trimethyl-5,6-dihydroquinazoline (reagent preparation 3) in step 1. ¹H NMR (400 MHz, d₆-DMSO) δ 8.34 (s, 1H), 7.94 (s, 2H), 7.66 (s, 1H), 7.62 (dd, 1H), 6.90 (d, 1H), 6.12 (s, 1H), 4.59 (s, 2H), 4.33 (m, 2H), 3.83 (m, 2H), 2.65 (s, 2H), 1.89 (s, 3H), 0.94 (s, 6H); MS (ES) for C₂₀H₂₄BN₃O₃: 366 (MH⁺).

[4-(6,6-dimethyl-5,6-dihydroquinazolin-4-yl)-2,3,4,5-tetrahydro-1,4-benzoxazepin-7-yl]boronic acid. Synthesized according to the method of reagent preparation 23 using 4-chloro-6,6-dimethyl-5,6-dihydroquinazoline (reagent preparation 3) in step 1. ¹H NMR (400 MHz, d₆-DMSO) δ 8.39 (s, 1H), 7.93 (s, 2H), 7.68 (s, 1H), 7.62 (dd, 1H), 6.89 (d, 1H), 6.29 (d, 1H), 6.23 (d, 1H), 4.61 (s, 2H), 4.32 (m, 2H), 3.84 (m, 2H), 2.69 (s, 2H), 0.97 (s, 6H); MS (ES) for C₁₉H₂₂BN₃O₃: 352 (MH⁺).

{4-[7-(methyloxy)quinazolin-4-yl]-2,3,4,5-tetrahydro-1,4-benzoxazepin-7-yl}boronic acid. Synthesized according to the method of reagent preparation 23 using 4-chloro-7-methoxyquinazoline (reagent preparation 1) in step 1. MS (ES) for C₁₈H₁₈BN₃O₄: 352 (MH⁺).

[4-(2,6,6-trimethyl-5,6,7,8-tetrahydroquinazolin-4-yl)-2,3,4,5-tetrahydro-1,4-benzoxazepin-7-yl]boronic acid. Synthesized according to the method of reagent preparation 23 using 4-chloro-2,6,6-trimethyl-5,6,7,8-tetrahydroquinazoline (reagent preparation 8) in step 1. MS (EI) for C₂₀H₂₆BN₃O₃: 368 (MH⁺).

{4-[(7S)-7-ethyl-2-methyl-5,6,7,8-tetrahydroquinazolin-4-yl]-2,3,4,5-tetrahydro-1,4-benzoxazepin-7-yl}boronic acid. Synthesized according to the method of reagent preparation 23 using (S)-4-chloro-7-ethyl-2-methyl-5,6,7,8-tetrahydroquinazoline (reagent preparation 8) in step 1. MS (ES) for C₂₀H₂₆BN₃O₃: 368 (MH⁺).

{4-[(7S)-7-ethyl-5,6,7,8-tetrahydroquinazolin-4-yl]-2,3,4,5-tetrahydro-1,4-benzoxazepin-7-yl}boronic acid. Synthesized according to the method of reagent preparation 23 using (S)-4-chloro-7-ethyl-5,6,7,8-tetrahydroquinazoline (reagent preparation 3) in step 1. ¹H NMR (400 MHz, d₆-DMSO) δ 8.33 (d, 1H), 7.96 (s, 2H), 7.68 (d, 1H), 7.61 (dd, 1H), 6.89 (d, 1H), 4.69 (d, 1H), 4.59 (d, 1H), 4.37 (dt, 1H), 4.25 (dt, 1H), 3.84 (t, 2H), 2.84 (dd, 1H), 2.75 (m, 1H), 2.46 (m, 1H), 2.26 (dd, 1H), 1.89 (m, 1H), 1.70 (m, 1H), 1.37 (m, 2H), 1.10 (m, 1H), 0.95 (t, 3H); MS (ES) for C₁₉H₂₄BN₃O₃: 354 (MH⁺).

[4-(6,6,8-trimethyl-5,6-dihydroquinazolin-4-yl)-2,3,4,5-tetrahydro-1,4-benzoxazepin-7-yl]boronic acid. Synthesized according to the method of reagent preparation 23 using 4-chloro-6,6,8-trimethyl-5,6-dihydroquinazoline (reagent preparation 3) in step 1. ¹H NMR (400 MHz, d₆-DMSO) δ 8.45 (s, 1H), 7.94 (s, 2H), 7.67 (d, 1H), 7.62 (dd, 1H), 6.90 (d, 1H), 5.99 (d, 1H), 4.59 (s, 2H), 4.32 (m, 2H), 3.83 (m, 2H), 2.66 (d, 2H), 1.97 (s, 3H), 0.93 (s, 6H); MS (ES) for C₂₀H₂₄BN₃O₃: 366 (MH⁺).

(4-{2-[(dimethylamino)methyl]-7-methoxy-6,6-dimethyl-5,6,7,8-tetrahydroquinazolin-4-yl}-2,3,4,5-tetrahydro-1,4-benzoxazepin-7-yl)boronic acid. Synthesized according to the method of reagent preparation 23 using 1-(4-chloro-7-methoxy-6,6-dimethyl-5,6,7,8-tetrahydroquinazolin-2-yl)-N,N-dimethylmethanamine (reagent preparation 17) in step 1. MS (ES) for C₂₃H₃₃BN₄O₄: 441 (MH⁺).

(4-{2-[(dimethylamino)methyl]-7-methoxyquinazolin-4-yl}-2,3,4,5-tetrahydro-1,4-benzoxazepin-7-yl)boronic acid. Synthesized according to the method of reagent preparation 23 using 1-(4-chloro-7-methoxyquinazolin-2-yl)-N,N-dimethylmethanamine (reagent preparation 20) in step 1. MS (ES) for C₂₁H₂₅BN₄O₄: 409 (MH⁺).

### Reagent Preparation 24

### N-(5-bromo-2-chloropyridin-3-yl)methanesulfonamide

STEP 1: A solution of 5-bromo-2-chloropyridin-3-amine (1.0 g, 4.8 mmol) and diisopropylethylamine (1.85 mL, 10.6 mmol) in dichloromethane (25 mL) was cooled to 0° C., and then methanesulfonyl chloride (750 uL, 9.6 mmol) was added slowly. The reaction mixture was stirred at 0° C. for 15 min and was then warmed to rt. After stirring for 2 h, water was added, and then the biphasic mixture was partitioned. The organic phase was dried over magnesium sulfate, filtered, and concentrated in vacuo. The residue was then dissolved in dioxane (10 mL) and water (10 mL). Potassium carbonate (2.76 g, 20 mmol) was added, and the reaction mixture was stirred for 15 h at rt. Water was then added to the mixture which was subsequently acidified with aqueous citric acid (10%). The aqueous mixture was extracted twice with ethyl acetate. The combined organic extracts were dried over magnesium sulfate, filtered, and concentrated in vacuo. The residue was purified by flash chromatography (gradient, 100% hexanes to 50% hexanes:50% ethyl acetate) to provide N-(5-bromo-2-chloropyridin-3-yl)methanesulfonamide (520 mg, 1.82 mmol, 38% yield) as a light pink solid. ¹H NMR (400 MHz, CDCl₃) δ 8.27 (d, 1H), 8.14 (d, 1H), 6.83 (br s, 1H), 3.11 (s, 3H); MS (EI) for C₆H₆BrClN₂O₂S: 285, 287, 289 (Br+Cl isotopes, MH⁺).

### Reagent Preparation 25

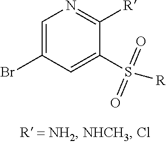

STEP 1: To a solution of (R)-pyrrolidin-3-ol (32 mg, 0.37 mmol) and potassium carbonate (102 mg, 0.74 mmol) in dioxane (2 mL) and water (400 uL) was added 2-amino-5-bromopyridine-3-sulfonyl chloride (100 mg, 0.37 mmol, prepared according to the methods in WO2008144463). The reaction mixture was stirred for 2 h at rt. Saturated sodium bicarbonate was then added, and the aqueous solution was extracted twice with ethyl acetate. The combined organic extracts were dried over magnesium sulfate, filtered, and concentrated in vacuo to provide (R)-1-(2-amino-5-bromopyridin-3-ylsulfonyl)pyrrolidin-3-ol (87.3 mg, 0.27 mmol, 73% yield) as a white solid. ¹H NMR (400 MHz, DMSO-D6-d₆) δ 8.31 (d, 1H), 7.92 (d, 1H), 6.85 (br s, 2H), 5.02 (br s, 1H), 4.23 (dt, 1H), 3.38-3.25 (m, 3H), 3.14-3.06 (m, 1H), 1.92-1.81 (m, 1H), 1.77-1.67 (m, 1H); MS (EI) for C₉H₁₂BrN₃O₃S: 322, 324 (Br isotopes, MH⁺).

Using analogous synthetic techniques and substituting with alternative starting reagents in step 1 the following reagents were prepared. Alternative starting materials were obtained commercially unless otherwise indicated.

2-amino-5-bromo-N-(2-methoxyethyl)pyridine-3-sulfonamide. Prepared according to the methods described in reagent preparation 25 using 2-methoxyethanamine in step 1.

2-amino-5-bromo-N-(2,2,2-trifluoroethyl)pyridine-3-sulfonamide. Prepared according to the methods described in reagent preparation 25 using 2,2,2-trifluoroethanamine in step 1.

2-amino-5-bromo-N-(2-hydroxyethyl)-N-methylpyridine-3-sulfonamide. Prepared according to the methods described in reagent preparation 25 using 2-(methylamino)ethanol in step 1.

2-amino-5-bromo-N-(2-hydroxypropyl)pyridine-3-sulfonamide. Prepared according to the methods described in reagent preparation 25 using 1-aminopropan-2-ol in step 1. MS (EI) for C₈H₁₂BrN₃O₃S: 310, 312 (Br isotopes, MH⁺).

2-amino-N-(azetidin-3-yl)-5-bromopyridine-3-sulfonamide. Prepared according to the methods described in reagent preparation 25 using tert-butyl 3-aminoazetidine-1-carboxylate in step 1.

2-amino-5-bromo-N-(2,3-dihydroxypropyl)pyridine-3-sulfonamide. Prepared according to the methods described in reagent preparation 25 using 3-aminopropane-1,2-diol in step 1. MS (EI) for C₈H₁₂BrN₃O₄S: 326, 328 (Br isotopes, MH⁺).

1-(2-amino-5-bromopyridin-3-ylsulfonyl)piperidin-3-ol. Prepared according to the methods described in reagent preparation 25 using piperidin-3-ol in step 1. MS (EI) for C₁₀H₁₄BrN₃O₃S: 336, 338 (Br isotopes, MH⁺).

2-amino-N-(3-amino-2,2-dimethylpropyl)-5-bromopyridine-3-sulfonamide. Prepared according to the methods described in reagent preparation 25 using 2,2-dimethylpropane-1,3-diamine in step 1. MS (EI) for C₁₀H₁₇BrN₄O₂S: 337, 339 (Br isotopes, MH⁺).

2-amino-5-bromo-N-(3-hydroxy-2,2-dimethylpropyl)pyridine-3-sulfonamide. Prepared according to the methods described in reagent preparation 25 using 3-amino-2,2-dimethylpropan-1-ol in step 1. MS (EI) for C₁₀H₁₆BrN₃O₃S: 338, 340 (Br isotopes, MH⁺).

2-amino-5-bromo-N-(1-hydroxy-2-methylpropan-2-yl)pyridine-3-sulfonamide. Prepared according to the methods described in reagent preparation 25 using 2-amino-2-methylpropan-1-ol in step 1. MS (EI) for C₉H₁₄BrN₃O₃S: 324, 326 (Br isotopes, MH⁺).

tert-butyl 4-((2-amino-5-bromopyridine-3-sulfonamido)methyl)piperidine-1-carboxylate. Prepared according to the methods described in reagent preparation 25 using tert-butyl 4-(aminomethyl)piperidine-1-carboxylate in step 1. MS (EI) for C₁₆H₂₅BrN₄O₄S: 393, 395 (Br isotopes, MH⁺-t-butyl).

2-amino-5-bromo-N-((1-methylpiperidin-4-yl)methyl)pyridine-3-sulfonamide. Prepared according to the methods described in reagent preparation 25 using (1-methylpiperidin-4-yl)methanamine in step 1. MS (EI) for C₁₂H₁₉BrN₄O₂S: 363, 365 (Br isotopes, MH⁺).

tert-butyl 1-((2-amino-5-bromopyridine-3-sulfonamido)methyl)cyclopropylcarbamate. Prepared according to the methods described in reagent preparation 25 using tert-butyl 1-(aminomethyl)cyclopropylcarbamate in step 1. MS (EI) for C₁₄H₂₁BrN₄O₄S: 365, 367 (Br isotopes, MH⁺-t-butyl).

tert-butyl trans-4-(2-amino-5-bromopyridine-3-sulfonamido)cyclohexylcarbamate. Prepared according to the methods described in reagent preparation 25 using tert-butyl trans-4-aminocyclohexylcarbamate in step 1.

benzyl 1-(2-amino-5-bromopyridine-3-sulfonamido)propan-2-ylcarbamate. Prepared according to the methods described in reagent preparation 25 using benzyl 1-aminopropan-2-ylcarbamate in step 1.

2-amino-5-bromo-N-ethylpyridine-3-sulfonamide. Prepared according to the methods described in reagent preparation 25 using ethylamine in step 1. ¹H NMR (400 MHz, CDCl₃) δ 8.28 (d, 1H), 8.07 (d, 1H), 5.63 (br s, 2H), 4.61 (t, 1H), 3.06-2.97 (m, 2H), 1.14 (t, 3H); MS (EI) for C₇H₁₀BrN₃O₂S: 280, 282 (Br isotopes, MH⁺).

2-amino-5-bromo-N-isopropylpyridine-3-sulfonamide. Prepared according to the methods described in reagent preparation 25 using isopropylamine in step 1. ¹H NMR (400 MHz, CDCl₃) δ 8.28 (d, 1H), 8.09 (d, 1H), 5.59 (br s, 2H), 4.52 (d, 1H), 3.50-3.39 (m, 1H), 1.11 (d, 6H); MS (EI) for C₈H₁₂BrN₃O₂S: 294, 296 (Br isotopes, MH⁺).

2-amino-5-bromo-N-(2-(dimethylamino)ethyl)pyridine-3-sulfonamide. Prepared according to the methods described in reagent preparation 25 using N,N-dimethylethane-1,2-diamine in step 1. ¹H NMR (400 MHz, CDCl₃) δ 8.27 (d, 1H), 8.08 (d, 1H), 5.66 (br s, 2H), 2.99-2.93 (m, 2H), 2.36-2.30 (m, 2H), 2.12 (s, 6H); MS (EI) for C₉H₁₅BrN₄O₂S: 323, 325 (Br isotopes, MH⁺).

2-amino-5-bromo-N-(2-hydroxyethyl)pyridine-3-sulfonamide. Prepared according to the methods described in reagent preparation 25 using 2-aminoethanol in step 1. ¹H NMR (400 MHz, CDCl₃) δ 8.29 (d, 1H), 8.08 (d, 1H), 5.65 (br s, 3H), 5.23 (br s, 1H), 3.76-3.67 (m, 3H), 3.16-3.07 (m, 3H); MS (EI) for C₇H₁₀BrN₃O₅S: 296, 298 (Br isotopes, MH⁺).

1-(2-amino-5-bromopyridin-3-ylsulfonyl)-3-(hydroxymethyl)azetidin-3-ol. Prepared according to the methods described in reagent preparation 25 using 3-(hydroxymethyl)azetidin-3-ol (prepared according to procedures described in WO2007044515) in step 1. ¹H NMR (400 MHz, CD₃OD) δ 8.28 (d, 1H), 8.00 (d, 1H), 3.90-3.84 (m, 2H), 3.70-3.64 (m, 2H), 3.32-3.29 (m, 2H); MS (EI) for C₉H₁₂BrN₃O₄S: 338, 340 (Br isotopes, MH⁺).

2-(2-amino-5-bromopyridine-3-sulfonamido)acetamide. Prepared according to the methods described in reagent preparation 25 using 2-aminoacetamide hydrochloride in step 1. ¹H NMR (400 MHz, DMSO-d₆) δ 8.26 (d, 1H), 8.18 (br s, 1H), 7.90 (d, 1H), 7.34 (br s, 1H), 7.12 (br s, 1H), 6.84 (br s, 2H), 3.45 (s, 2H); MS (EI) for C₇H₉BrN₄O₃S: 309, 311 (Br isotopes, MH⁺).

tert-butyl 3-(2-amino-5-bromopyridine-3-sulfonamido)-2-hydroxypropylcarbamate. Prepared according to the methods described in reagent preparation 25 using tert-butyl 3-amino-2-hydroxypropylcarbamate in step 1. ¹H NMR (400 MHz, DMSO-d6) δ 8.26 (d, 1H), 7.88 (d, 1H), 6.82 (br s, 2H), 6.74 (t, 1H), 5.02 (d, 1H), 3.50-3.42 (m, 1H), 2.88 (t, 2H), 2.82 (dd, 1H), 2.57 (dd, 1H), 1.37 (s, 9H); MS (EI) for C₁₃H₂₁BrN₄O₅S: 369, 371 (Br isotopes, MH⁺-t-Bu).

5-bromo-3-(3-(dimethylamino)azetidin-1-ylsulfonyl)pyridin-2-amine. Prepared according to the methods described in reagent preparation 25 using N,N-dimethylazetidin-3-amine hydrochloride in step 1. ¹H NMR (400 MHz, DMSO-d6) δ 8.39 (d, 1H), 7.92 (d, 1H), 6.90 (br s, 2H), 3.88-3.76 (m, 2H), 3.63-3.54 (m, 2H), 3.07-2.97 (m, 1H), 1.96 (s, 6H); MS (EI) for C₁₀H₁₅BrN₄O₂S: 335, 337 (Br isotopes, MH⁺).

5-bromo-N-(2-hydroxyethyl)-2-(methylamino)pyridine-3-sulfonamide. Prepared according to the methods described in reagent preparation 25 using 5-bromo-2-(methylamino)pyridine-3-sulfonyl chloride (prepared from 5-bromo-N-methylpyridin-2-amine using analogous conditions to those described in WO2008144463) and 2-aminoethanol in step 1. ¹H NMR (400 MHz, CDCl₃) δ 8.28 (d, 1H), 8.00 (d, 1H), 7.10-7.03 (m, 1H), 6.48-6.39 (m, 1H), 3.93 (t, 1H), 3.60 (q, 2H), 3.04-2.96 (m, 5H); MS (EI) for C₈H₁₂BrN₃O₃S: 310, 312 (Br isotopes, MH⁺).

N-(1-(2-amino-5-bromopyridin-3-ylsulfonyl)azetidin-3-yl)-N-methyl-2-nitrobenzenesulfonamide. Prepared according to the methods described in reagent preparation 25 using N-(azetidin-3-yl)-N-methyl-2-nitrobenzenesulfonamide in step 1. ¹H NMR (400 MHz, CDCl₃) δ 8.32 (d, 1H), 8.06-8.03 (m, 1H), 8.00 (d, 1H), 7.77-7.72 (m, 2H), 7.70-7.65 (m, 1H), 5.78 (br s, 2H), 4.90-4.80 (m, 1H), 4.19-4.08 (m, 2H), 4.01 (dd, 2H), 2.91 (s, 31.1); MS (EI) for C₁₅H₁₆BrN₅O₆S: 506, 508 (Br isotopes, MH⁺).

tert-butyl 4-(2-amino-5-bromopyridin-3-ylsulfonyl)piperazine-1-carboxylate. Prepared according to the methods described in reagent preparation 25 using tert-butyl piperazine-1-carboxylate in step 1. ¹H NMR (400 MHz, DMSO-d6) δ 8.34 (d, 1H), 7.86 (d, 1H), 6.90 (br s, 2H), 3.40-3.35 (m, 4H), 3.09-3.02 (m, 4H), 1.37 (s, 9H); MS (EI) for C₁₄H₂₁BrN₄O₄S: 367, 365 (Br isotopes, MH⁺-t-Bu).

3-(3-amino-3-methylazetidin-1-ylsulfonyl)-5-bromopyridin-2-amine. Prepared according to the methods described in reagent preparation 25 using 3-methylazetidin-3-amine hydrochloride (prepared by procedures described in WO2007007057 followed by benzylidene deprotection) in step 1. ¹H NMR (400 MHz, DMSO-d6) δ 8.37 (d, 1H), 7.88 (d, 1H), 6.86 (br s, 2H), 3.58-3.47 (m, 4H), 2.06 (br s, 2H), 1.22 (s, 3H); MS (EI) for C₉H₁₃BrN₄O₂S: 321, 323 (Br isotopes, MH⁺).

tert-butyl 2-(2-amino-5-bromopyridine-3-sulfonamido)-2-methylpropylcarbamate. Prepared according to the methods described in reagent preparation 25 using tert-butyl 2-amino-2-methylpropylcarbamate in step 1. ¹H NMR (400 MHz, CDCl₃) δ 8.26 (d, 1H), 8.08 (d, 1H), 5.89 (br s, 1H), 5.60 (br s, 2H), 5.04 (t, 1H), 3.12 (d, 2H), 1.46 (s, 9H), 1.19 (s, 6H); MS (EI) for C₁₄H₂₃BrN₄O₄S: 367, 369 (Br isotopes, MH⁺-t-Bu).

tert-butyl 5-((2-amino-5-bromopyridine-3-sulfonamido)methyl)hexahydrocyclopenta[c]pyrrole-2(1H)-carboxylate. Prepared according to the methods described in reagent preparation 25 using tert-butyl 5-(aminomethyl)hexahydrocyclopenta[c]pyrrole-2(1H)-carboxylate (prepared from substrates described in WO2004006846) in step 1. ¹H NMR (400 MHz, CDCl₃) δ 8.28 (d, 1H), 8.06 (d, 1H), 5.65 (br s, 2H), 5.03 (t, 1H), 3.41 (br s, 2H), 3.17 (br s, 2H), 2.93 (t, 2H), 2.63-2.54 (m, 2H), 2.14-1.98 (m, 3H), 1.46 (s, 9H), 1.09-0.98 (m, 2H); MS (EI) for C₁₈H₂₇BrN₄O₄S: 419, 421 (Br isotopes, MH⁺-t-Bu).

tert-butyl 1-(2-amino-5-bromopyridine-3-sulfonamido)butan-2-ylcarbamate. Prepared according to the methods described in reagent preparation 25 using tert-butyl 1-aminobutan-2-ylcarbamate in step 1. ¹H NMR (400 MHz, DMSO-d6) δ 8.28 (d, 1H), 7.89 (d, 1H), 6.78 (br s, 2H), 6.57 (d, 1H), 3.33-3.26 (m, 1H), 2.77-2.65 (m, 2H), 1.53-1.39 (m, 1H), 1.37 (s, 9H), 1.28-1.15 (m, 1H), 0.76 (t, 3H); MS (EI) for C₁₄H₂₃BrN₄O₄S: 367, 369 (Br isotopes, MH⁺-t-Bu).

tert-butyl 4-(2-amino-5-bromopyridine-3-sulfonamido)-2-methylbutan-2-ylcarbamate. Prepared according to the methods described in reagent preparation 25 using tert-butyl 4-amino-2-methylbutan-2-ylcarbamate in step 1. ¹H NMR (400 MHz, CDCl₃) δ 8.27 (d, 1H), 8.06 (d, 1H), 5.64 (br s, 2H), 5.07 (br s, 1H), 4.41 (br s, 1H), 2.98 (q, 2H), 1.93-1.85 (m, 2H), 1.41 (s, 9H), 1.22 (s, 6H); MS (EI) for C₁₅H₂₅BrN₄O₄S: 381, 383 (Br isotopes, MH⁺-t-Bu).

2-amino-N-(2-amino-2-methylpropyl)-5-bromopyridine-3-sulfonamide. Prepared according to the methods described in reagent preparation 25 using 2-methylpropane-1,2-diamine in step 1. ¹H NMR (400 MHz, CDCl₃) δ 8.27 (d, 1H), 8.07 (d, 1H), 5.69 (br s, 2H), 2.73 (s, 2H), 1.12 (s, 6H); MS (EI) for C₉H₁₅BrN₄O₂S: 323, 325 (Br isotopes, MH⁺).

tert-butyl 1-(2-amino-5-bromopyridin-3-ylsulfonyl)azetidin-3-ylcarbamate. Prepared according to the methods described in reagent preparation 25 using tert-butyl azetidin-3-ylcarbamate in step 1. ¹H NMR (400 MHz, CDCl₃) δ 8.31 (d, 1H), 8.00 (d, 1H), 5.76 (br s, 2H), 4.80 (br s, 1H), 4.50-4.36 (m, 1H), 4.11 (t, 2H), 3.75 (t, 2H), 1.42 (s, 9H).; MS (EI) for C₁₃H₁₉BrN₄O₄S: 407, 409 (Br isotopes, MH⁺).

tert-butyl 1-(2-amino-5-bromopyridin-3-ylsulfonyl)piperidin-4-ylcarbamate sulfonamide. Prepared according to the methods described in reagent preparation 25 using tert-butyl piperidin-4-ylcarbamate in step 1.

2-amino-5-bromo-N-(2-hydroxy-2-methylpropyl)pyridine-3-sulfonamide. Prepared according to the methods described in reagent preparation 25 using 1-amino-2-methylpropan-2-ol in step 1.

2-Amino-5-bromo-N,N-dimethylpyridine-3-sulfonamide. Prepared according to the method of reagent preparation 25 by using dimethylamine in step 1. MS (EI) for C₇H₁₀BrN₃O₂S: 280 (MH⁺).

5-Bromo-3-(morpholinosulfonyl)pyridin-2-amine. Prepared according to the method of reagent preparation 25 by using morpholine in step 1. MS (EI) for C₉H₁₂BrN₃O₃S: 322 (MH⁺).

5-Bromo-3-(4-methylpiperazin-1-ylsulfonyl)pyridin-2-amine. Prepared according to the method of reagent preparation 25 by using N-methylpiperazine in step 1. MS (EI) for C₁₀H₁₅BrN₄O₂S: 335 (MH⁺).

3-(Azetidin-1-ylsulfonyl)-5-bromopyridin-2-amine. Prepared according to the method of reagent preparation 25 by using N-methylpiperazine in step 1. MS (EI) for C₈H₁₀BrN₃O₂S: 292 (MH⁺).

2-Amino-5-bromo-N-methylpyridine-3-sulfonamide. Prepared according to the method of reagent preparation 25 by using methylamine in step 1. MS (EI) for C₆H₈BrN₃O₂S: 266 (MH⁺).

1-(2-Amino-5-bromopyridin-3-ylsulfonyl)azetidin-3-ol. Prepared according to the method of reagent preparation 25 by using azetidinol in step 1. MS (EI) for C₈H₁₀BrN₃O₃S: 308 (MH⁺).

5-Bromo-3-(pyrrolidin-1-ylsulfonyl)pyridin-2-amine. Prepared according to the method of reagent preparation 25 by using pyrrolidine in step 1. MS (EI) for C₉H₁₂BrN₃O₂S: 306 (MH⁺).

1-(2-Amino-5-bromopyridin-3-ylsulfonyl)pyrrolidin-3-ol. Prepared according to the method of reagent preparation 25 by using 3-pyrrolidinol in step 1. MS (EI) for C₉H₁₂BrN₃O₃S: 322 (MH⁺).

2-Amino-5-bromo-N-cyclobutylpyridine-3-sulfonamide. Prepared according to the method of reagent preparation 25 by using cyclobutylamine in step 1. MS (EI) for C₉H₁₂BrN₃O₂S: 306 (MH⁺).

2-Amino-5-bromopyridine-3-sulfonamide. Prepared according to the method of reagent preparation 25 by using ammoniumhydroxide in step 1. MS (EI) for C₅H₆BrN₃O₂S: 252 (MH⁺).

2-Amino-5-bromo-N-ethyl-N-methylpyridine-3-sulfonamide. Prepared according to the method of reagent preparation 25 by using N-methylethylamine in step 1. MS (EI) for C₈H₁₂BrN₃O₂S: 294 (MH⁺).

5-Bromo-3-(3,3-difluoroazetidin-1-ylsulfonyl)pyridin-2-amine. Prepared according to the method of reagent preparation 25 by using 3,3-difluoroazetidine in step 1. MS (EI) for C₈H₈BrF₂N₃O₂S: 328 (MH⁺).

2-Amino-5-bromo-N-(1-hydroxypropan-2-yl)pyridine-3-sulfonamide. Prepared according to the method of reagent preparation 25 by using 2-aminopropan-1-ol in step 1. MS (EI) for C₈H₁₂BrN₃O₃S: 310 (MH⁺).

2-Amino-5-bromo-N-(2-fluoroethyl)pyridine-3-sulfonamide. Prepared according to the method of reagent preparation 25 by using 2-fluoroethylamine in step 1. MS (EI) for C₇H₉BrFN₃O₂S: 298 (MH⁺).

tert-Butyl 1-(2-amino-5-bromopyridin-3-ylsulfonyl)pyrrolidin-3-ylcarbamate. Prepared according to the method of reagent preparation 25 by using tert-butyl pyrrolidin-3-ylcarbamate in step 1. MS (EI) for C₁₄H₂₁BrN₄O₄S: 365 (MH⁺-tBu).

1-(2-Amino-5-bromopyridin-3-ylsulfonyl)piperidin-4-ol. Prepared according to the method of reagent preparation 25 by using 4-hydroxypiperidine in step 1. MS (EI) for C₁₀H₁₄BrN₃O₃S: 336 (MH⁺)

tert-Butyl 1-(2-amino-5-bromopyridin-3-ylsulfonyl)piperidin-3-ylcarbamate. Prepared according to the method of reagent preparation 25 by using tert-butyl piperidin-3-ylcarbamate in step 1. MS (EI) for C₁₅H₂₃BrN₄O₄S: 379 (MH⁺-tBu).

tert-Butyl 2-(2-amino-5-bromopyridine-3-sulfonamido)ethylcarbamate. Prepared according to the method of reagent preparation 25 by using tert-butyl 2-aminoethylcarbamate in step 1. MS (EI) for C₁₂H₁₉BrN₄O₄S: 339 (MH⁺-tBu).

2-Amino-5-bromo-N-(3-hydroxypropyl)pyridine-3-sulfonamide. Prepared according to the method of reagent preparation 25 by using 3-hydroxypropylamine in step 1. MS (EI) for C₈H₁₂BrN₃O₃S: 310 (MH⁺).

tert-Butyl 3-(2-amino-5-bromopyridine-3-sulfonamido)propylcarbamate. Prepared according to the method of reagent preparation 25 by using tert-butyl 2-aminopropylcarbamate in step 1. MS (EI) for C₁₃H₂₁BrN₄O₄S: 353 (MH⁺-tBu).

2-Amino-5-bromo-N-(3,3,3-trifluoro-2-hydroxypropyl)pyridine-3-sulfonamide. Prepared according to the method of reagent preparation 25 by using 3-amino-1,1,1-trifluoropropan-2-ol in step 1. MS (EI) for C₈H₉BrF₃N₃O₃S: 364 (MH⁺).

tert-Butyl 5-(2-amino-5-bromopyridin-3-ylsulfonyl)hexahydropyrrolo[3,4-c]pyrrole-2(1H)-carboxylate. Prepared according to the method of reagent preparation 25 by using tert-butyl hexahydropyrrolo[3,4-c]pyrrole-2(1H)-carboxylate in step 1. MS (EI) for C₁₆H₂₃BrN₄O₄S: 391 (MH⁺-tBu)

tert-Butyl 1-(2-amino-5-bromopyridin-3-ylsulfonyl)-3-methylpyrrolidin-3-ylcarbamate. Prepared according to the method of reagent preparation 25 by using tert-butyl 3-methylpyrrolidin-3-ylcarbamate in step 1. MS (EI) for C₁₅H₂₃BrN₄O₄S: 379 (MH⁺-tBu).

(1S,4S)-tert-Butyl 5-(2-amino-5-bromopyridin-3-ylsulfonyl)-2,5-diazabicyclo[2.2.1]heptane-2-carboxylate. Prepared according to the method of reagent preparation 25 by using (1S,4S)-tert-butyl 2,5-diazabicyclo[2.2.1]heptane-2-carboxylate in step 1. MS (EI) for C₁₅H₂₁BrN₄O₄S: 377 (MH⁺-tBu).

(R)-tert-Butyl 2-((2-amino-5-bromopyridine-3-sulfonamido)methyl)pyrrolidine-1-carboxylate. Prepared according to the method of reagent preparation 25 by using (R)-tert-butyl 2-(aminomethyl)pyrrolidine-1-carboxylate in step 1. MS (EI) for C₁₅H₂₃BrN₄O₄S: 335 (MH⁺-Boc).

(S)-tert-Butyl 2-((2-amino-5-bromopyridine-3-sulfonamido)methyl)pyrrolidine-1-carboxylate. Prepared according to the method of reagent preparation 25 by using (S)-tert-butyl 2-(aminomethyl)pyrrolidine-1-carboxylate in step 1. MS (EI) for C₁₅H₂₃BrN₄O₄S: 335 (MH⁺-Boc).

(1R,4R)-tert-Butyl 5-(2-amino-5-bromopyridin-3-ylsulfonyl)-2,5-diazabicyclo[2.2.1]heptane-2-carboxylate. Prepared according to the method of reagent preparation 25 by using (1R,4R)-tert-butyl 2,5-diazabicyclo[2.2.1]heptane-2-carboxylate in step 1. MS (EI) for C₁₅H₂₁BrN₄O₄S: 377 (MH⁺-Boc).

tert-Butyl 4-(2-amino-5-bromopyridine-3-sulfonamido)piperidine-1-carboxylate. Prepared according to the method of reagent preparation 25 by using tert-butyl 4-aminopiperidine-1-carboxylate in step 1. MS (EI) for C₁₅H₂₃BrN₄O₄S: 379 (MH⁺-Boc).

5-Bromo-3-((1S,4S)-5-methyl-2,5-diazabicyclo[2.2.1]heptan-2-ylsulfonyl)pyridin-2-amine. Prepared according to the method of reagent preparation 25 by using (1S,4S)-2-methyl-2,5-diazabicyclo[2.2.1]heptane in step 1. MS (EI) for C₁₁H₁₅BrN₄O₂S: 347 (MH⁺).

(S)-tert-Butyl 1-(2-amino-5-bromopyridin-3-ylsulfonyl)pyrrolidin-3-ylcarbamate. Prepared according to the method of reagent preparation 25 by using (S)-tert-butyl pyrrolidin-3-ylcarbamate in step 1. MS (EI) for C₁₄H₂₁BrN₄O₄S: 421 (MH⁺).

(R)-tert-Butyl 1-(2-amino-5-bromopyridin-3-ylsulfonyl)pyrrolidin-3-ylcarbamate. Prepared according to the method of reagent preparation 25 by using (R)-tert-butyl pyrrolidin-3-ylcarbamate in step 1. MS (EI) for C₁₄H₂₁BrN₄O₄S: 421 (MH⁺).

tert-Butyl 8-(2-amino-5-bromopyridin-3-ylsulfonyl)-8-azabicyclo[3.2.1]octan-3-ylcarbamate. Prepared according to the method of reagent preparation 25 by using tert-butyl 8-azabicyclo[3.2.1]octan-3-ylcarbamate (WO 2009055077) in step 1. MS (EI) for C₁₇H₂₅BrN₄O₄S: 461 (MH⁺).

2,2,2-Trichloroethyl 3-(2-amino-5-bromopyridine-3-sulfonamido)-8-azabicyclo[3.2.1]octane-8-carboxylate. Prepared according to the method of reagent preparation 25 by using 2,2,2-trichloroethyl 3-amino-8-azabicyclo[3.2.1]octane-8-carboxylate (WO 2009055077) in step 1. MS (EI) for C₁₅H₁₈BrCl₃N₄O₄S: 535 (MH⁺).

(R)-tert-Butyl 3-((2-amino-5-bromopyridine-3-sulfonamido)methyl)pyrrolidine-1-carboxylate. Prepared according to the method of reagent preparation 25 by using (S)-tert-butyl 3-(aminomethyl)pyrrolidine-1-carboxylate in step 1. MS (EI) for C₁₅H₂₃BrN₄O₄S: 435 (MH⁺).

(S)-tert-Butyl 3-((2-amino-5-bromopyridine-3-sulfonamido)methyl)pyrrolidine-1-carboxylate. Prepared according to the method of reagent preparation 25 by using (R)-tert-butyl 3-(aminomethyl)pyrrolidine-1-carboxylate in step 1. MS (EI) for C₁₅H₂₃BrN₄O₄S: 435 (MH⁺).

(R)-tert-Butyl 3-(2-amino-5-bromopyridine-3-sulfonamido)pyrrolidine-1-carboxylate. Prepared according to the method of reagent preparation 25 by using (R)-tert-butyl 3-aminopyrrolidine-1-carboxylate in step 1. MS (EI) for C₁₄H₂₁BrN₄O₄S: 421 (MH⁺).

(S)-tert-Butyl 3-(2-amino-5-bromopyridine-3-sulfonamido)pyrrolidine-1-carboxylate. Prepared according to the method of reagent preparation 25 by using (S)-tert-butyl 3-aminopyrrolidine-1-carboxylate in step 1. MS (EI) for C₁₄H₂₅BrN₄O₄S: 421 (MH⁺).

tert-Butyl 3-((2-amino-5-bromopyridine-3-sulfonamido)methyl)piperidine-1-carboxylate. Prepared according to the method of reagent preparation 25 by using tert-butyl 3-(aminomethyl)piperidine-1-carboxylate in step 1. MS (EI) for C₁₆H₂₅BrN₄O₄S: 449 (MH⁺).

tert-Butyl 2-((2-amino-5-bromopyridine-3-sulfonamido)methyl)piperidine-1-carboxylate. Prepared according to the method of reagent preparation 25 by using tert-butyl 2-(aminomethyl)piperidine-1-carboxylate in step 1. MS (EI) for C₁₆H₂₅BrN₄O₄S: 449 (MH⁺).

(R)-tert-Butyl 3-((2-amino-5-bromopyridine-3-sulfonamido)methyl)piperidine-1-carboxylate. Prepared according to the method of reagent preparation 25 by using (S)-tert-butyl 3-(aminomethyl)piperidine-1-carboxylate in step 1. MS (EI) for C₁₆H₂₅BrN₄O₄S: 449 (MH⁺).

(S)-tert-Butyl 3-((2-amino-5-bromopyridine-3-sulfonamido)methyl)piperidine-1-carboxylate. Prepared according to the method of reagent preparation 25 by using (R)-tert-butyl 3-(aminomethyl)piperidine-1-carboxylate in step 1. MS (EI) for C₁₆H₂₅BrN₄O₄S: 449 (MH⁺).

(S)-2-amino-5-bromo-N-((1-methylpiperidin-3-yl)methyl)pyridine-3-sulfonamide. Prepared according to the method of reagent preparation 25 by using (R)-(1-methylpiperidin-3-yl)methanamine in step 1. MS (EI) for C₁₂H₁₉BrN₄O₂S: 363 (MH⁺).

2-amino-5-bromo-N-[(3R)-1-methylpyrrolidin-3-yl]pyridine-3-sulfonamide. Synthesized according to the method of reagent preparation 25 by using (R)-1-methylpyrrolidin-3-amine hydrochloride (synthesized according to the method of Journal of Medicinal Chemistry (2002), 45(3), 721-739) in step 1. MS (EI) for C₁₀H₁₅BrN₄O₂S: 334, 336 (MH⁺, Br isotope pattern).

2-amino-5-bromo-N-{[(3S)-1-methylpyrrolidin-3-yl]methyl}pyridine-3-sulfonamide. Synthesized according to the method of reagent preparation 25 by using (R)-(1-methylpyrrolidin-3-yl)methanamine hydrobromide (synthesized according to the methods of WO 2006028904 for the synthesis of benzyl[[(R)-1-(tert-butoxycarbonyl)pyrrolidin-3-yl]methyl]carbamate, WO 2006002047 for the synthesis of (S)-benzyl pyrrolidin-3-ylmethylcarbamate and Journal of Medicinal Chemistry (2002), 45(3), 721-739 for the synthesis of (R)-benzyl (1-methylpyrrolidin-3-yl)methylcarbamate, using (R)-3-(aminomethyl)-1-(tert-butyloxycarbonyl)pyrrolidine as starting material) in step 1. MS (EI) for C₁₁H₁₇BrN₄O₂S: 348, 350 (MH⁺, Br isotope pattern).

tert-Butyl 6-(2-amino-5-bromopyridin-3-ylsulfonyl)-2,6-diazaspiro[3.3]heptane-2-carboxylate. Prepared according to the method of reagent preparation 25 by using tert-butyl 2,6-diazaspiro[3.3]heptane-2-carboxylate in step 1. MS (EI) for C₁₅H₂₁BrN₄O₄S: 377 (MH⁺-tBu).

(S)-tert-Butyl 1-(5-bromo-2-chloropyridin-3-ylsulfonyl)pyrrolidin-3-ylcarbamate. Prepared according to the methods described in reagent preparation 25 using 5-bromo-2-chloropyridine-3-sulfonyl chloride and (S)-tert-butyl pyrrolidin-3-ylcarbamate in step 1. ¹H NMR (400 MHz, CDCl₃) δ 8.61 (d, 1H), 8.52 (d, 1H), 4.67 (s, 1H), 4.25 (s, 1H), 3.57 (m, 4H), 3.34 (m, 1H), 2.22 (m, 1H), 1.92 (m, 1H), 1.45 (s, 9H); MS (ES) for C₁₄H₁₉BrClN₃O₄S: 440, 442 (Br isotopes, MH⁺).

tert-Butyl 3-((2-amino-5-bromopyridine-3-sulfonamido)methyl)azetidine-1-carboxylate. Prepared according to the methods described in reagent preparation 25 using tert-butyl 3-(aminomethyl)azetidine-1-carboxylate in step 1. MS (ES) for C₁₄H₂₁BrN₄O₄S: 421, 423 (Br isotopes, MH⁺).

### Reagent Preparation 26

### N-(5-bromo-2-methylpyridin-3-yl)methanesulfonamide

STEP 1: A solution of 5-bromo-2-methylpyridin-3-amine (187 mg, 1.0 mmol) and diisopropylethylamine (523 uL, 3.0 mmol) in dichloromethane (5 mL) was cooled to 0° C., and then methanesulfonyl chloride (155 uL, 2.0 mmol) was added slowly. The reaction mixture was stirred at 0° C. for 8 min and was then warmed to it. After stirring for 1 h, the volatile materials were removed in vacuo. The residue was then dissolved in methanol (2.5 mL) and aqueous sodium hydroxide (2 M, 1.5 mL, 3 mmol) was added. The reaction mixture was stirred for 1 h 40 min at rt. Water was then added to the mixture which was subsequently extracted twice with dichloromethane. The combined organic extracts were extracted with aqueous citric acid (10%). The organic phase was discarded, and the aqueous phase was basified to pH ˜7.5 with aqueous sodium hydroxide (1 M). The aqueous mixture was extracted three times with dichloromethane. The combined organic extracts were dried over magnesium sulfate, filtered, and concentrated in vacuo. The residue was purified by flash chromatography (50% hexanes:50% ethyl acetate) to provide N-(5-bromo-2-methylpyridin-3-yl)methanesulfonamide (111 mg, 0.42 mmol, 42% yield) as a white solid. ¹H NMR (400 MHz, DMSO-d6) δ 9.58 (s, 1H), 8.44 (d, 1H), 7.87 (d, 1H), 3.10 (s, 3H), 2.47 (s, 3H); MS (EI) for C₇H₉BrN₂O₂S: 265, 267 (Br isotopes, MH⁺).

Using analogous synthetic techniques and substituting with alternative starting reagents in step 1 the following reagents were prepared. Alternative starting materials were obtained commercially unless otherwise indicated.

N-(5-Bromo-2-chlorophenyl)methanesulfonamide. Prepared according to the methods described in reagent preparation 26 using 5-bromo-2-chloroaniline in step 1. ¹H NMR (400 MHz, CDCl₃) δ 7.83 (d, 1H), 7.32-7.23 (m, 2H), 6.80 (br s, 1H), 3.06 (s, 3H); MS (EI) for C₇H₇BrClNO₂S: 282, 284, 286 (Br+Cl isotopes, MH⁺).

N-(5-Bromo-2-methoxypyridin-3-yl)methanesulfonamide. Prepared according to the methods described in reagent preparation 26 using 5-bromo-2-methoxypyridin-3-amine in step 1. ¹H NMR (400 MHz, CDCl₃) δ 7.97 (d, 1H), 7.90 (d, 1H), 6.73 (br s, 1H), 4.00 (s, 3H), 3.05 (s, 3H); MS (EI) for C₇H₉BrN₂O₃S: 281, 283 (Br isotopes, MH⁺).

N-(5-Bromo-2-cyanopyridin-3-yl)methanesulfonamide. Prepared according to the methods described in reagent preparation 26 using 3-amino-5-bromopicolinonitrile in step 1. ¹H NMR (400 MHz, CDCl₃) δ 8.55 (d, 1H), 8.29 (d, 1H), 7.00 (br s, 1H), 3.21 (s, 3H); MS (EI) for C₇H₆BrN₃O₂S: 276, 278 (Br isotopes, MH⁺).

N-(5-Bromopyridin-3-yl)methanesulfonamide. Prepared according to the methods described in reagent preparation 26 using 5-bromopyridin-3-amine in step 1. MS (EI) for C₆H₇BrN₂O₂S: 251, 253 (Br isotopes, MH⁺).

N-(5-Bromo-2-chloropyridin-3-yl)-2-chloro-6-methylbenzenesulfonamide. Prepared according to the methods described in reagent preparation 26 using 5-bromo-2-chloropyridin-3-amine and 2-chloro-6-methylbenzene-1-sulfonyl chloride in step 1. MS (EI) for C₁₂H₉BrCl₂N₂O₂S: 393, 395, 397 (Br+Cl isotopes, MH⁺).

N-(5-Bromo-2-fluoropyridin-3-yl)methanesulfonamide. Prepared according to the methods described in reagent preparation 26 using 5-bromo-2-fluoropyridin-3-amine in step 1. MS (EI) for C₆H₆BrFN₂O₂S: 269, 271 (Br isotopes, MH⁺).

N-(5-Bromo-2-chloropyridin-3-yl)acetamide. Prepared according to the methods described in reagent preparation 26 using 5-bromo-2-chloropyridin-3-amine and acetyl chloride in step 1.

Methyl 5-bromo-2-chloropyridin-3-ylcarbamate. Prepared according to the methods described in reagent preparation 26 using 5-bromo-2-chloropyridin-3-amine and methyl chloroformate in step 1.

### Reagent Preparation 27

### 5-bromo-2-chloro-3-(methylsulfonylmethyl)pyridine

STEP 1: A mixture of 5-bromo-2-chloro-3-(chloromethyl)pyridine (124 mg, 0.52 mmol) and sodium methanesulfinate (52 mg, 0.52 mmol) in dioxane (1.4 mL) and water (1.4 mL) was heated to 110° C. in a microwave reactor for 15 min. After cooling to rt, water was added to the reaction mixture which was subsequently extracted twice with ethyl acetate. The combined organic extracts were dried over magnesium sulfate, filtered, and concentrated in vacuo to provide 5-bromo-2-chloro-3-(methylsulfonylmethyl)pyridine (140 mg, 0.49 mmol, 94% yield) as a yellow solid. ¹H NMR (400 MHz, DMSO-d6) δ 8.63 (d, 1H), 8.21 (d, 1H), 4.70 (s, 2H), 3.10 (s, 3H); MS (EI) for C₇H₇BrClNO₂S: 284, 286, 288 (Br+CI isotopes, MH⁺).

Using analogous synthetic techniques and substituting with alternative starting reagents in step 1 the following reagent was prepared. Alternative starting materials were obtained commercially unless otherwise indicated.

5-Bromo-3-(methylsulfonylmethyl)pyridin-2-amine. Prepared according to the methods described in reagent preparation 27 using 5-bromo-3-(bromomethyl)pyridin-2-amine hydrochloride in step 1. ¹H NMR (400 MHz, DMSO-d₆) δ 8.03 (d, 1H), 7.59 (d, 1H), 6.35 (br s, 2H), 4.44 (s, 2H), 2.95 (s, 3H); MS (EI) for C₇H₉BrN₂O₂S: 265, 267 (Br isotopes, MH⁺).

### Reagent Preparation 28

### N-(5-bromo-2-chloropyridin-3-yl)-N-methylmethanesulfonamide

STEP 1: A solution of N-(5-bromo-2-chloropyridin-3-yl)methanesulfonamide (96 mg, 0.34 mmol, reagent preparation 24) in DMF (1 mL) was treated with potassium carbonate (93 mg, 0.68 mmol) and iodomethane (33 uL, 0.51 mmol) at rt for 18 h. Water was then added, and the resulting aqueous mixture was extracted twice with ethyl acetate. The combined organic extracts were washed with aqueous lithium chloride (10%) followed by water, dried over magnesium sulfate, filtered, and concentrated in vacuo to provide N-(5-bromo-2-chloropyridin-3-yl)-N-methylmethanesulfonamide (91.2 mg, 0.304 mmol, 90% yield) as a light yellow solid. ¹H NMR (400 MHz, CDCl₃) δ 8.46 (d, 1H), 8.00 (d, 1H), 3.32 (s, 3H), 3.07 (s, 3H); MS (EI) for C₇H₈BrClN₂O₂S: 299, 301, 303 (Br+Cl isotopes, MH⁺).

### Reagent Preparation 29

### 5-bromo-2-chloro-3-(difluoromethoxy)pyridine

To a solution of 5-bromo-2-chloropyridin-3-ol (150 mg, 0.72 mmol) in DMF (5 mL) was added potassium carbonate (298 mg, 2.2 mmol). The mixture was heated to 70° C. and bromodifluoromethane was bubbled through for 3 min. After cooling to rt, water was added, and the resulting aqueous mixture was extracted twice with ethyl acetate. The organic extracts were washed with aqueous lithium chloride (10%) followed by water, dried over magnesium sulfate, filtered, and concentrated in vacuo to provide 5-bromo-2-chloro-3-(difluoromethoxy)pyridine (159 mg, 0.61 mmol, 85% yield) as a brown oil. ¹H NMR (400 MHz, CDCl₃) δ 8.36 (d, 1H), 7.76 (d, 1H), 6.61 (t, 1H); MS (EI) for C₆H₃BrClF₂NO: 258 (M⁺).

### Reagent Preparation 30

### N-(5-bromo-2-ethoxypyridin-3-yl)methanesulfonamide

STEP 1: A solution of 5-bromo-2-chloro-3-nitropyridine (100 mg, 0.42 mmol) and 1,8-diazabicyclo[5.4.0]undec-7-ene (315 uL, 2.11 mmol) in ethanol (1 mL) was heated to 50° C. for 50 min and then cooled to rt. Water was added and the resulting aqueous mixture was extracted twice with ethyl acetate. The combined organic extracts were washed with 1 N HCl, dried over magnesium sulfate, filtered, and concentrated in vacuo. The residue was purified by flash chromatography (gradient, 100% hexanes to 90% hexanes:10% ethyl acetate) to provide 5-bromo-2-ethoxy-3-nitropyridine (52.2 mg, 0.211 mmol, 50% yield) as a yellow oil. ¹H NMR (400 MHz, CDCl₃) δ 8.42 (d, 1H), 8.36 (d, 1H), 4.55 (q, 2H), 1.45 (t, 3H); MS (EI) for C₇H₇BrN₂O₃: 246, 248 (M).

STEP 2: To a solution of 5-bromo-2-ethoxy-3-nitropyridine (75.2 mg, 0.304 mmol) in ethyl acetate (3 mL) was added tin(II) chloride (289 mg, 1.52 mmol), and the mixture was heated to reflux for 2 h. After cooling to rt, 50% aqueous sodium hydroxide was added dropwise until a sticky brown solid completely formed. Sodium sulfate was then added, and the mixture was stirred for several minutes. The solids were then removed by filtration. The filtrate was dried over sodium sulfate, filtered, and concentrated in vacuo to provide 5-bromo-2-ethoxypyridin-3-amine (53 mg, 0.25 mmol, 80% yield) as a dark blue film. ¹H NMR (400 MHz, CDCl₃) δ 7.56 (d, 1H), 6.97 (d, 1H), 4.37 (q, 2H), 3.85 (br s, 2H), 1.40 (dd, 3H); MS (EI) for C₇H₉BrN₂O: 217, 219 (Br isotopes, MH⁺).

STEP 3: A solution of 5-bromo-2-ethoxypyridin-3-amine (53 mg, 0.25 mmol) and diisopropylethylamine (96 uL, 0.55 mmol) in dichloromethane (1 mL) was cooled to 0° C. and methanesulfonyl chloride (39 uL, 0.5 mmol) was added. The mixture was allowed to warm to rt over 15 h, and then water was added. The resulting mixture was extracted with dichloromethane. The organic extract was dried over magnesium sulfate, filtered, and concentrated in vacuo. The residue was dissolved in methanol (500 uL) and dioxane (500 uL), and then sodium hydroxide (2 M, 190 uL, 0.38 mmol) was added. The mixture was heated to 60° C. and 3 drops of aqueous sodium hydroxide (50%) were added. After stirring a further 30 min, the mixture was cooled to rt. Dilution with water was followed by acidification with aqueous citric acid (10%) and then two extractions with ethyl acetate. The combined organic extracts were washed with water, dried over magnesium sulfate, filtered, and concentrated in vacuo. The residue was purified by flash chromatography (gradient 100% hexanes to 70% hexanes:30% ethyl acetate) to provide N-(5-bromo-2-ethoxypyridin-3-yl)methanesulfonamide (32.1 mg, 0.11 mmol, 43% yield) as a colorless film. ¹H NMR (400 MHz, CDCl₃) δ 7.95 (d, 1H), 7.89 (d, 1H), 6.75 (br s, 1H), 4.42 (q, 2H), 3.05 (s, 3H), 1.41 (t, 3H); MS (EI) for C₃H₁₁BrN₂O₃S: 295, 297 (Br isotopes, MH⁺).

Using analogous synthetic techniques and substituting with alternative starting reagents in step 1 the following reagent was prepared. Alternative starting materials were obtained commercially unless otherwise indicated.

N-(2-(Benzyloxy)-5-bromopyridin-3-yl)methanesulfonamide. Prepared according to the methods described in reagent preparation 30 using benzyl alcohol in step 1. ¹H NMR (400 MHz, CDCl₃) δ 8.00 (d, 1H), 7.91 (d, 1H), 7.44-7.34 (m, 5H), 6.71 (br s, 1H), 5.40 (s, 2H), 2.99 (s, 3H); MS (EI) for C₁₃H₁₃BrN₂O₃S: 357, 359 (Br isotopes, MH⁺).

### Reagent Preparation 31

### N-(2-amino-5-bromopyridin-3-yl)methanesulfonamide

STEP 1: To a solution of 5-bromo-3-nitropyridin-2-amine (218 mg, 1 mmol) in THF (5 mL) was added DMAP (183 mg, 1.5 mmol) and di-tert-butyl dicarbonate (655 mg, 3 mmol). After stirring 40 min at rt, the volatile materials were removed in vacuo, and the resulting residue was purified by flash chromatography (gradient, 100% hexanes to 70% hexanes:30% ethyl acetate). The isolated material indicated the addition of two Boc groups by ¹H NMR. This material was dissolved in ethyl acetate (8 mL) and was treated with excess N,N-dimethylethylenediamine. After stirring for 17 h at rt, the reaction mixture was diluted with ethyl acetate. The resulting solution was washed with aqueous citric acid (10%) followed by water, dried over magnesium sulfate, filtered, and concentrated in vacuo to provide tert-butyl 5-bromo-3-nitropyridin-2-ylcarbamate (270 mg, 0.85 mmol, 85% yield) as an orange solid. ¹H NMR (400 MHz, CDCl₃) δ 9.48 (br s, 1H), 8.74 (d, 1H), 8.63 (d, 1H), 1.56 (s, 9H); MS (EI) for C₁₀H₁₂BrN₃O₄: 316, 318 (Br isotopes, M−H).

STEP 2: Iron powder (293 mg, 5.2 mmol) was added to a solution of tert-butyl 5-bromo-3-nitropyridin-2-ylcarbamate (167 mg, 0.52 mmol) in acetic acid (2.5 mL). The mixture was stirred at 60° C. for 1 h 20 min before cooling to rt. The mixture was then diluted with ethyl acetate, and solids were removed by filtration through celite. The filtrate was washed with water followed by saturated aqueous sodium bicarbonate. The organic phase was dried over magnesium sulfate, filtered, and concentrated in vacuo to provide tert-butyl 3-amino-5-bromopyridin-2-ylcarbamate (96.3 mg, 0.33 mmol, 64% yield) as a gray solid. ¹H NMR (400 MHz, CDCl₃) δ 7.83 (d, 1H), 7.20 (d, 1H), 6.95 (br s, 1H), 4.42 (br s, 2H), 1.51 (s, 9H); MS (EI) for C₁₀H₁₄BrN₃O₂: 232, 234 (Br isotopes, MH⁺-t-butyl).

STEP 3: A solution of tert-butyl 3-amino-5-bromopyridin-2-ylcarbamate (96.3 mg, 0.33 mmol) and diisopropylethylamine (128 uL, 074 mmol) in dichloromethane (2 mL) was cooled to 0° C., and to it was added methanesulfonyl chloride (52 uL, 0.67 mmol). The mixture was allowed to warm to rt over 2 h. The mixture was then diluted with dichloromethane and was then washed with aqueous citric acid (10%) followed by water. The organic phase was then dried over magnesium sulfate, filtered, and concentrated in vacuo. The residue was purified by flash chromatography (gradient, 100% hexanes to 70% hexanes:30% ethyl acetate) to provide tert-butyl 5-bromo-3-(N-(methylsulfonyl)methylsulfonamido)pyridin-2-ylcarbamate (77 mg, 0.17 mmol, 52% yield) as a colorless film. ¹H NMR (400 MHz, CDCl₃) δ 8.64 (d, 1H), 7.79 (d, 1H), 7.10 (s, 1H), 3.44 (s, 6H), 1.52 (s, 9H); MS (EI) for C₁₂H₁₈BrN₃O₆S₂: 388, 390 (Br isotopes, MH⁺-t-butyl).

STEP 4: A solution of tert-butyl 5-bromo-3-(N-(methylsulfonyl)methylsulfonamido)pyridin-2-ylcarbamate (68 mg, 0.15 mmol) and N,N-dimethylethylenediamine (169 uL, 1.5 mmol) in dioxane (1 mL) was stirred at rt for 70 min. After diluting with ethyl acetate, the mixture was washed with aqueous citric acid (10%) followed by water. The organic phase was then dried over magnesium sulfate, filtered, and concentrated in vacuo. The residue was then diluted with dichloromethane which was then washed with 1 N HCl. After partitioning, the organic phase was dried over magnesium sulfate, filtered, and concentrated in vacuo to provide tert-butyl 5-bromo-3-(methylsulfonamido)pyridin-2-ylcarbamate (57 mg, 0.15 mmol, quantitative yield) as a white solid. ¹H NMR (400 MHz, CDCl₃) δ 8.24 (d, 1H), 8.07 (d, 1H), 2.98 (s, 3H), 1.54 (s, 9H); MS (EI) for C₁₀H₁₆BrN₃O₄S: 310, 312 (Br isotopes, MH⁺-t-butyl).

STEP 5: A solution of tert-butyl 5-bromo-3-(methylsulfonamido)pyridin-2-ylcarbamate (57 mg, 0.15 mmol) in methanol (1 mL) and HCl (4 M in dioxane, 375 uL, 1.5 mmol) was heated to 60° C. for 90 min. The volatile materials were then removed in vacuo to provide N-(2-amino-5-bromopyridin-3-yl)methanesulfonamide as its hydrochloride salt in quantitative yield. ¹H NMR (400 MHz, DMSO-d6) δ 9.10 (br s, 1H), 7.95 (d, 1H), 7.54 (d, 1H), 6.42 (br s, 1H), 3.02 (s, 3H); MS (EI) for C₆H₈BrN₃O₂S: 266, 268 (Br isotopes, MH⁺).

### Reagent Preparation 32

### 5-bromo-1-(tetrahydro-2H-pyran-2-yl)-1H-pyrazolo[3,4-b]pyridine

To a solution of 5-bromo-1H-pyrazolo[3,4-b]pyridine (1.4 g, 7.2 mmol) and dihydropyran (3.3 mL, 36.0 mmol) in tetrahydrofuran (20 mL) was added (±)-camphorsulfonic acid (250 mg) and the reaction mixture was stirred at 65° C. for 16 hours. After cooling to room temperature it was diluted with ethyl acetate (250 mL), washed with saturated aqueous sodium bicarbonate (2×100 mL) and brine (100 mL), dried over sodium sulfate, filtered and concentrated. Gradient column chromatography (10% to 30% ethyl acetate in hexane) provided 5-bromo-1-(tetrahydro-2H-pyran-2-yl)-1H-pyrazolo[3,4-b]pyridine (1.8 g, 90%). MS (EI) for C₁₁H₁₂BrN₃O: 283 (MH⁺).

### Reagent Preparation 33

### 2-Amino-5-bromo-N,N-dimethylnicotinamide

To a suspension of 2-amino-5-bromonicotinic acid (0.35 g, 1.61 mmol) in tetrahydrofuran (5 mL) was added dimethylamine (0.8 mL of a 2M solution in tetrahydrofuran, 1.60 mmol), diethylphosphoryl cyanide (0.29 g, 1.77 mmol), and triethylamine (0.34 g, 3.38 mmol) at 0° C. The mixture was stirred at 0° C. for 30 min and then at room temperature for 4 h. Concentration and purification by column chromatography on silica (5-10% methanol in dichloromethane) gave the title Compound as a white solid. MS (EI) for C₈H₁₀BrN₃O: 244 (MH⁺).

### Reagent Preparation 34

### 5-Bromo-3-(ethylsulfonyl)pyridin-2-amine

STEP 1: 2-Amino-5-bromopyridine-3-sulfonyl chloride (94 mg, 0.35 mmol) was taken into THF (2 mL) followed by addition of anhydrous hydrazine (40 uL, 1.4 mmol) and the mixture was stirred for 10 minutes at room temperature. The mixture was concentrated and dried to give 2-amino-5-bromopyridine-3-sulfonohydrazide as a white solid, which was then taken into ethanol (2 mL) followed by addition of sodium acetate (320 mg, 3.9 mmol) and ethyl iodide (140 uL, 1.75 mmol). The mixture was refluxed for 12 h then cooled to room temperature and concentrated. The residue was partitioned with ethyl acetate and water and the organic phase washed with brine then dried over sodium sulfate, filtered and concentrated to give 5-bromo-3-(ethylsulfonyl)pyridin-2-amine (67 mg, 72%) as a yellow oil. MS (EI) for C₇H₉N₂SO₂Br: 265, 267 (MH⁺).

Using analogous synthetic techniques and substituting with alternative starting reagents in step 1 the following reagents were prepared.

5-Bromo-3-(methylsulfonyl)pyridin-2-amine. Synthesized according to the method of reagent preparation 34 using iodomethane. GCMS (EI) for C₆H₇N₂SO₂Br: 250, 252 (M⁺).

3-(2-amino-5-bromopyridin-3-ylsulfonyl)propane-1,2-diol. Synthesized according to the method of reagent preparation 34 using 3-bromopropane-1,2-diol followed by silica gel chromatography using ethyl ether then ethyl acetate as eluent. MS (EI) for C₇H₉N₂SO₂Br: 311, 313 (MH⁺).

3-(2-amino-5-bromopyridin-3-ylsulfonyl)propan-1-ol. Synthesized according to the method of reagent preparation 34 using 3-bromopropan-1-ol followed by silica gel chromatography using ethyl ether as eluent. MS (EI) for C₇H₉N₂SO₂Br: 295, 297 (MH⁺).

(S)-3-(2-amino-5-bromopyridin-3-ylsulfonyl)-2-methylpropan-1-ol. Synthesized according to the method of reagent preparation 34 using (S)-3-bromo-2-methylpropan-1-ol followed by silica gel chromatography using 4:1 ethyl ether:hexanes as eluent. MS (EI) for C₇H₉N₂SO₂Br: 309, 311 (MH⁺).

(R)-3-(2-amino-5-bromopyridin-3-ylsulfonyl)-2-methylpropan-1-ol. Synthesized according to the method of reagent preparation 34 using (R)-3-bromo-2-methylpropan-1-ol followed by silica gel chromatography using 4:1 ethyl ether:hexanes as eluent. MS (EI) for C₇H₉N₂SO₂Br: 309, 311 (MH⁺).

### Reagent Preparation 35

### 6-bromo-2-methyl-1-({[2-(trimethylsilyl)ethyl]oxy}methyl)-1H-imidazo[4,5-b]pyridine

To a solution of 6-bromo-2-methyl-1H-imidazo[4,5-b]pyridine (3.0 g, 14.1 mmol) in a mixture of N,N-dimethylformamide and tetrahydrofuran (30 mL, 2:1) at 0° C. was added 60% sodium hydride in mineral oil (0.68 g, 17.0 mmol) and the reaction mixture was stirred for 30 minutes, followed by the addition of 2-(trimethylsilyl)ethoxymethyl chloride (2.7 mL, 14.9 mmol). The reaction mixture was stirred for 16 hours at room temperature then it was quenched by the careful addition of water and diluted with ethyl acetate (250 mL), washed with brine (3×150 mL), dried over sodium sulfate, filtered and concentrated. Gradient column chromatography (10% to 30% ethyl acetate in hexane) provided 6-bromo-2-methyl-1-({[2-(trimethylsilyl)ethyl]oxy}methyl)-1H-imidazo[4,5-b]pyridine (4.4 g, 92%). ¹H NMR (400 MHz, CDCl₃): 8.41 (s, 1H), 8.12 (s, 1H), 5.67 (s, 2H), 3.62 (m, 2H), 2.76 (s, 3H), 0.96 (m, 2H), 0.00 (s, 9H). MS (EI) for C₁₃H₂₀BrN₃OSi: 342, 344 (MH⁺, Br iotope pattern).

### Reagent Preparation 36

### 6-bromo-N-ethyl-3-(methoxymethyl)-3H-imidazo[4,5-b]pyridin-2-amine and 6-bromo-N-ethyl-N,3-bis(methoxymethyl)-3H-imidazo[4,5-b]pyridin-2-amine

Step 1: To a cooled (0° C.) solution of 5-bromopyridine-2,3-diamine (5.0 g, 27 mmol) in NMP (20 mL) was added isothiocyanatoethane (2.3 mL, 26 mmol). The resulting solution was heated (65° C.) for four hours and then cooled to ambient temperature before 1,3-diisopropylcarbodiimide (4.2 mL, 27 mmol) was added. The reaction mixture was stirred for 18 hours, diluted with water and the resulting suspension was collected by filtration. Trituration with ethyl acetate provided 6-bromo-N-ethyl-3H-imidazo[4,5-b]pyridin-2-amine (4.8 g, 75% yield) as a brown solid. ¹H NMR (400 MHz, d₆-DMSO) δ 11.41 (bs, 1H), 7.91 (s, 1H), 7.53 (s, 1H), 7.17 (s, 1H), 3.33 (q, 2H), 1.17 (t, 3H); MS (ES) for C₈H₉BrN₄: 241 (MH⁺).

Step 2: To a cooled (0° C.) solution of 6-bromo-N-ethyl-3H-imidazo[4,5-b]pyridin-2-amine (0.36 g, 1.5 mmol) in DMF was added NaH (60% dispersion in mineral oil, 0.060 g, 1.5 mmol) portionwise over 15 minutes. The reaction mixture was stirred for 15 minutes and then chloro(methoxy)methane (0.12 mL, 1.5 mmol) was added dropwise over 15 minutes. The resulting slurry was allowed to warm to ambient temperature and was stirred for two hours and was partitioned between ethyl acetate and saturated aqueous sodium bicarbonate. The organic layer was washed with brine, dried over magnesium sulfate, filtered and concentrated in vacuo. Purification by silica gel chromatography provided both 6-bromo-N-ethyl-N,3-bis(methoxymethyl)-3H-imidazo[4,5-b]pyridin-2-amine (0.091 g, 18%) and 6-bromo-N-ethyl-3-(methoxymethyl)-3H-imidazo[4,5-b]pyridin-2-amine (0.15 g, 35% yield). Bisprotected product: MS (ES) for C₁₂H₁₇BrN₄O₁: 329 (MH⁺). Monoprotected product: ¹H NMR (400 MHz, CDCl₃) δ 8.03 (d, 1H), 7.73 (d, 1H), 5.42 (s, 2H), 4.98 (s, 1H), 3.59 (q, 2H), 3.36 (s, 3H), 1.34 (t, 3H); MS (ES) for C₁₀H₁₃BrN₄O: 285 (MH⁺).

### Reagent Preparation 37

### 7-Bromo-2H-pyrido[2,3-e][1,2,4]thiadiazin-3(4H)-one 1,1-dioxide

STEP 1: 2-Amino-5-bromopyridine-3-sulfonyl chloride (reagent preparation 25) (95.5 mg, 0.35 mmol) was treated with 0.5M ammonia in dioxane solution (7 mL) and the mixture was stirred for 1 h at room temperature. Concentrated aqueous ammonia (2 mL) was then added to the mixture then stirred an additional 12 h. The mixture was then concentrated and the residue suspended in water (5 mL). The solid was collected by filtration and dried to give 2-amino-5-bromopyridine-3-sulfonamide (55.7 mg, 89%).

STEP 2: 2-Amino-5-bromopyridine-3-sulfonamide as obtained above (0.22 mmol) was taken into THF (2 mL) followed by addition of diisopropylethylamine (115 uL, 0.66 mmol). Phosgene (20 W % in toluene, 120 uL, 0.22 mmol) was added carefully and the mixture was allowed to stir for 1 h at room temperature. The mixture was partitioned with ethyl acetate and 0.5M aqueous hydrochloric acid. The organic phase was then extracted once with saturated aqueous sodium bicarbonate. The organic layer was discarded and the aqueous phase carefully acidified to pH 1-2 with concentrated aqueous hydrochloric acid. The aqueous mixture was then extracted once with ethyl acetate, dried over sodium sulfate, filtered and concentrated to give 7-bromo-2H-pyrido[2,3-e][1,2,4]thiadiazin-3(4H)-one 1,1-dioxide (17.3 mg, 28%) as a solid. MS (EI) for C₆H₄N₃O₃SBr: 277, 279 (M⁻).

### Reagent Preparation 38

### 2-amino-5-bromopyridine-3-sulfonic acid

STEP 1: 2-Amino-5-bromopyridine-3-sulfonyl chloride (100 mg, 0.37 mmol) was taken into 1:1 aqueous dioxane (3 mL) and the mixture was basified to pH 14 by drop wise addition of 50% aqueous sodium hydroxide solution. The mixture was warmed to 75° C. for 0.5 h then cooled to room temperature and concentrated. The residue was taken into water (2 mL) and carefully acidified to pH 1-2 by concentrated aqueous hydrochloric acid addition and cooled to 0° C. After 1 h at 0° C. the crystalline solid obtained was collected by filtration and dried to give 2-amino-5-bromopyridine-3-sulfonic acid as a solid. ¹H NMR (DMSO-d₆): 8.24 (d, 1H), 8.06 (d, 1H). MS (EI) for C₅H₅N₂SO₃Br: 253, 255 (MH⁺, Br pattern).

### Reagent Preparation 39

### N-(5-bromo-2-(dimethylamino)pyridin-3-yl)methanesulfonamide

STEP 1: 5-Bromo-2-chloro-3-nitropyridine (J. Heterocyclic Chem. 2003, 40, 261) (128 mg, 0.54 mmol) was taken into THF (0.25 mL) followed by addition of 40 W % aqueous dimethylamine (0.25 mL) and the resulting solution was stirred for 1 h at room temperature. The mixture was then partitioned with ethyl ether and 1 M aqueous hydrochloric acid. The organic solution was then washed with additional 1 M aqueous hydrochloric acid (3×) then dried over magnesium sulfate, filtered and concentrated to give 5-bromo-N,N-dimethyl-3-nitropyridin-2-amine. MS (EI) for C₇H₈N₃O₂Br: 246, 248 (MH⁺, Br pattern).

STEP 2: 5-Bromo-N,N-dimethyl-3-nitropyridin-2-amine as obtained in step 1 (0.54 mmol) was taken into ethyl acetate (10 mL) followed by addition of tin (II) chloride (522 mg, 2.8 mmol) and the mixture was heated to reflux for 15 minutes then cooled to room temperature. 50 W % aqueous sodium hydroxide was added drop wise to the mixture until a precipitate formed then solid sodium sulfate was added. The mixture was filtered and the filter cake washed with ethyl acetate. The organic filtrate was concentrated to give 5-bromo-N2,N2-dimethylpyridine-2,3-diamine (53 mg, 45%) was an amorphous residue. MS (EI) for C₇H₁₀N₃Br: 216, 218 (MH⁺, Br pattern).

STEP 3: 5-Bromo-N2,N2-dimethylpyridine-2,3-diamine (53 mg, 0.25 mmol) was taken into THF (2 mL) followed by addition of diisopropylethylamine (213 uL, 1.25 mmol) and methanesulfonyl chloride (95 ul, 1.25 mmol). The mixture was allowed to stir for 48 h at room temperature then partitioned with ethyl acetate and water. The organic phase was washed with brine then dried over sodium sulfate, filtered and concentrated. The residue was taken into methanol (3 mL) followed by addition of potassium hydroxide (108 mg, 10 eq) in a minimum of water. The mixture was stirred for 15 minutes at room temperature then partitioned with ethyl acetate and 10% aqueous citric acid. The organic solution was dried over magnesium sulfate, filtered and concentrated. The residue was purified by silica gel chromatography to give N-(5-bromo-2-(dimethylamino)pyridin-3-yl)methanesulfonamide (27.9 mg, 39%). MS (EI) for C₈H₁₂N₃SO₂Br: 294, 296 (MH⁺, Br pattern).

Using analogous synthetic techniques and substituting with alternative starting reagents in step 1 the following reagents were prepared.

N-(2-(Benzylamino)-5-bromopyridin-3-yl)methanesulfonamide. Synthesized according to the method of reagent preparation 39 using benzylamine in step 1. MS (EI) for C₁₃H₁₄N₃SO₂Br: 356, 358 (MH⁺, Br pattern).

N-(5-Bromo-2-(phenylamino)pyridin-3-yl)methanesulfonamide. Synthesized according to the method of reagent preparation 39 using neat aniline at 75° C. in step 1. MS (EI) for C₁₂H₁₂N₃SO₂Br: 342, 344 (MH⁺, Br pattern).

N-(5-Bromo-2-(methylamino)pyridin-3-yl)methanesulfonamide. Synthesized according to the method of reagent preparation 39 using methyamine in step 1. MS (EI) for C₇H₁₀N₃SO₂Br: 280, 282 (MH⁺, Br pattern).

### Reagent Preparation 40

### 1,1-dimethylethyl{(3S)-1-[(5-bromo-2-hydroxypyridin-3-yl)sulfonyl]pyrrolidin-3-yl}carbamate and 1,1-dimethylethyl[(3S)-1-({5-bromo-2-[(3S)-3-({[(1,1-dimethylethyl)oxy]carbonyl}amino)pyrrolidin-1-yl]pyridin-3-yl}sulfonyl)pyrrolidin-3-yl]carbamate

STEP 1: To a solution of 3-amino-5-bromo-2-chloropyridine (0.23 g, 1.1 mmol) in acetonitrile (3.0 mL) at −15° C. was added a solution of sodium ntrite (0.091 g, 1.3 mmol) in water (1.20 mL), followed by the addition of concentrate hydrochloric acid (1.8 mL, 21.3 mmol) and the reaction mixture was stirred for 5 minutes. A 30 wt % solution of sulfur dioxide in acetic acid 3.0 mL, 1.3 mmol) was prepared and introduced into the reaction mixture, followed by the addition of a solution of copper(II) chloride 0.091 g, 0.68 mmol) in water (1.2 mL). The stirring was continued for an additional 3 hours at −5° C. The pH of the mixture was adjusted to 8 by the addition of a solution of potassium hydrogenphosphate and 2M aqueous sodium hydroxide and partitioned with ethyl acetate (50 mL). The organic layer was separated and washed with water (10 mL) and brine (10 mL), dried over sodium sulfate, filtered and concentrated to give 5-bromo-2-chloropyridine-3-sulfonyl chloride (0.20 g, 63%).

STEP 2: A mixture of 5-bromo-2-chloropyridine-3-sulfonyl chloride (0.19 g, 0.65 mmol), (3S)-(−)-3-(tert-butoxycarbonylamino)pyrrolidine (0.18 g, 0.98 mmol) and N,N-diisopropylethylamine (0.34 mL, 1.95 mmol) in dichloromethane (1.5 mL) was stirred for 16 hours at room temperature. The reaction mixture was partitioned between dichloromethane (50 mL) and brine (10 mL.). The organic layer was separated, dried over sodium sulfate, filtered and concentrated. The resulting crude product was dissolved in a mixture of 1,4-dioxane (1.5 mL) and 2M aqueous sodium hydroxide (1.5 mL) and stirred at 100° C. for 2 hours. After cooling to room temperature the reaction mixture was concentrated and the residue was partitioned between brine (20 mL) and ethyl acetate (50 mL). The organic layer was separated and washed with brine (20 mL), dried over sodium sulfate, filtered and concentrated. Gradient flash chromatography (25% to 50% ethyl acetate in hexane) followed by 10% methanol in dichloromethane provided 1,1-dimethylethyl[(3S)-1-({5-bromo-2-[(3S)-3-({[(1,1-dimethylethyl)oxy]carbonyl}amino)pyrrolidin-1-yl]pyridin-3-yl}sulfonyl)pyrrolidin-3-yl]carbamate (80 mg, 21%), MS (EI) for C₂₃H₃₆BrN₅O₆S: 591 (MH⁺); and 1,1-dimethylethyl {(3S)-1-[(5-bromo-2-hydroxypyridin-3-yl)sulfonyl]pyrrolidin-3-yl}carbamate (35 mg, 13%); MS (EI) for C₁₄H₂₀BrN₃O₅S: 423 (MH⁺).

### Reagent Preparation 41

### 4-[(2-amino-5-bromopyridin-3-yl)sulfonyl]-2-methylbutan-2-ol and 4-[(2-amino-5-bromopyridin-3-yl)sulfinyl]-2-methylbutan-2-ol

STEP 1: To a solution of 2-amino-5-bromopyridine-3-sulfonyl chloride (reagent preparation 25, step 1) (0.40 g, (1.47 mmol) in a mixture of 1,4-dioxane (8.0 mL) and water (1.0 mL) was added triphenylphosphine (1.64 g, 6.25 mmol) and the reaction mixture was stirred for 50 minutes at room temperature. Potassium carbonate (0.35 g, 2.50 mmol) was introduced, followed by 4-bromo-2-methyl-2-butanol (Tetrahedron Letters 2000, 41(38), 7337-7340) (0.31 g, 1.86 mmol) and the reaction mixture was stirred at 80° C. for 16 hours. After cooling to room temperature the reaction mixture was concentrated and the residue was partitioned between brine (50 mL) and ethyl acetate (100 mL). The organic layer was separated and washed with brine (50 mL), dried over sodium sulfate, filtered and concentrated. Gradient flash chromatography (25% to 50% ethyl acetate in hexane) provided 4-[(2-amino-5-bromopyridin-3-yl)thio]-2-methylbutan-2-ol (0.18 g, 42%); MS (EI) for C₁₀H₁₅BrN₂OS: 292 (MH⁺).

STEP 2A: To a solution of 4-[(2-amino-5-bromopyridin-3-yl)thio]-2-methylbutan-2-ol (90 mg, 0.31 mmol) in a mixture of methanol (750 μL), acetone (750 μL) and water (450 μL) was added potassium peroxymonosulfate (285 mg, 0.46 mmol) and the reaction mixture was stirred for 15 minutes at room temperature. The reaction mixture was partitioned between water (20 mL) and ethyl acetate (50 mL). The organic layer was separated and washed with water (20 mL) and brine (20 mL), dried over sodium sulfate, filtered and concentrated. Purification by flash chromatography (35% to 80% ethyl acetate in hexane) gave 4-[(2-amino-5-bromopyridin-3-yl)sulfonyl]-2-methylbutan-2-ol (48 mg, 48%); MS (EI) for C₁₀H₁₅BrN₂O₃S: 323 (MH⁺).

STEP 2B: To a solution of 4-[(2-amino-5-bromopyridin-3-yl)thio]-2-methylbutan-2-ol (83 mg, 0.28 mmol) in a mixture of methanol (750 μL), acetone (750 μL) and water (450 μL) was added potassium peroxymonosulfate (131 mg, 0.21 mmol) and the reaction mixture was stirred for 90 minutes at 0° C. The reaction mixture was partitioned between water (20 mL) and ethyl acetate (50 mL). The organic layer was separated and washed with water (20 mL) and brine (20 mL), dried over sodium sulfate, filtered and concentrated. Purification by flash chromatography (35% to 80% ethyl acetate in hexane) gave 4-[(2-amino-5-bromopyridin-3-yl)sulfinyl]-2-methylbutan-2-ol (52 mg, 60%); MS (EI) for C₁₀H₁₅BrN₂O₂S: 308 (MH⁺).

Using analogous synthetic techniques and substituting with alternative starting materials in step 1 the following reagents of the invention were prepared. Alternative starting materials were obtained commercially unless otherwise indicated.

(2S)-3-[(2-amino-5-bromopyridin-3-yl)sulfonyl]-2-methylpropan-1-ol. Prepared according to the method of reagent preparation 41 by using (S)-(+)-3-bromo-2-methyl-1-propanol in step 1. MS (EI) for C₉H₁₃BrN₂O₃S: 310 (MH⁺).

(2S)-3-[(2-amino-5-bromopyridin-3-yl)sulfinyl]-2-methylpropan-1-ol. Prepared according to the method of reagent preparation 41 by using (S)-(+)-3-bromo-2-methyl-1-propanol in step 1. MS (EI) for C₉H₁₃BrN₂O₂S: 294 (MH⁺).

### Reagent Preparation 42

### (4-chloro-5,6,7,8-tetrahydroquinazolin-7-yl)methanol

Ozone was bubbled through a cooled (−78° C.) solution of 4-chloro-7-vinyl-5,6,7,8-tetrahydroquinazoline (reagent preparation 3, 0.35 g, 1.8 mmol) in methanol (5 mL) and dichloromethane (30 mL) until a blue color persisted. The solution was then sparged with N₂ for 10 minutes and sodium borohydride (0.14 g, 3.6 mmol) was added portionwise. After 30 minutes the reaction mixture was partitioned between dichloromethane and saturated aqueous sodium bicarbonate. The organic layer was washed with brine, dried over magnesium sulfate, filtered and then concentrated in vacuo to provide (4-chloro-5,6,7,8-tetrahydroquinazolin-7-yl)methanol (0.32 g, 90% yield) as a waxy solid. MS (ES) for C₉H₁₁ClN₂O: 199 (MH⁺).

### Reagent example 43

### 1-(4-chloro-5,6,7,8-tetrahydroquinazolin-7-yl)ethanol

Step 1: Ozone was bubbled through a cooled (−78° C.) solution of 4-chloro-7-vinyl-5,6,7,8-tetrahydroquinazoline (reagent preparation 3, 0.38 g, 2.0 mmol) in dichloromethane (45 mL) until a blue color persisted. The solution was then sparged with N₂ for 10 minutes and triphenylphosphine (0.52 g, 2.0 mmol) was added portionwise. After one hour, the reaction mixture was partitioned between dichloromethane and saturated aqueous sodium bicarbonate. The organic layer was washed with brine, dried over magnesium sulfate, filtered and then concentrated in vacuo. Purification by silica gel chromatography provided 4-chloro-5,6,7,8-tetrahydroquinazoline-7-carbaldehyde (0.33 g, 85% yield) as a viscous oil. MS (ES) for C₉H₉ClN₂O: 197 (MH⁺).

Step 2: To a cooled (0° C.) solution of 4-chloro-5,6,7,8-tetrahydroquinazoline-7-carbaldehyde (0.10 g, 0.51 mmol) in THF (5 mL) was added a solution of MeMgBr (3.0 M in ethyl ether, 0.40 mL, 1.2 mmol). The resulting mixture was stirred at ambient temperature for 30 minutes and then partitioned between ethyl acetate and saturated sodium bicarbonate. The organic layer was washed with brine, dried over magnesium sulfate, filtered and concentrated in vacuo. Purification by silica gel chromatography provided 1-(4-chloro-5,6,7,8-tetrahydroquinazolin-7-yl)ethanol (0.09 g, 83% yield) as a waxy solid. MS (ES) for C₁₀H₁₃ClN₂O: 213 (MH⁺).

### Reagent example 44

### 4-chloro-7-(methoxymethyl)-5,6,7,8-tetrahydroquinazoline

To a slurry of (4-chloro-5,6,7,8-tetrahydroquinazolin-7-yl)methanol (reagent preparation 42, 0.80 g, 0.40 mmol), potassium carbonate (0.11 g, 0.81 mmol) and THF (15 mL) was added iodomethane (0.09 mL, 0.60 mmol). The reaction mixture was stirred for 18 hours and then partitioned between ethyl acetate and water. The organic layer was washed with brine, dried over magnesium sulfate, filtered and concentrated in vacuo. Purification by silica gel chromatography provided 4-chloro-7-(methoxymethyl)-5,6,7,8-tetrahydroquinazoline (0.03 g, 35% yield) as a waxy solid. MS (ES) for C₁₀H₁₃ClN₂O: 213 (MH⁺)

### Reagent Preparation 45

### 2-(azidomethyl)-4-chloro-6,6-dimethyl-5,6,7,8-tetrahydroquinazoline

STEP 1: To a solution of 2-(chloromethyl)-6,6-dimethyl-5,6,7,8-tetrahydroquinazolin-4(3H)-one (150 mg, 0.66 mmol, reagent preparation 17) in DMF (3 mL) was added sodium azide (215 mg, 3.3 mmol). The resulting mixture was stirred at rt for 35 min. Water was added and the resulting mixture was extracted twice with ethyl acetate. The combined organic extracts were washed with aqueous lithium chloride (10%), dried over magnesium sulfate, filtered, and concentrated in vacuo to provide 2-(azidomethyl)-6,6-dimethyl-5,6,7,8-tetrahydroquinazolin-4(3H)-one (151 mg, 0.65 mmol, 98% yield) as a waxy yellow solid. ¹H NMR (400 MHz, CDCl₃) δ 11.70 (br s, 1H), 4.41 (s, 2H), 2.66 (t, 2H), 2.33 (s, 2H), 1.58 (t, 3H), 1.00 (s, 6H); MS (EI) for C₁₁H₁₅N₅O: 234 (MH⁺).

STEP 2: A solution of 2-(azidomethyl)-6,6-dimethyl-5,6,7,8-tetrahydroquinazolin-4(3H)-one (151 mg, 0.65 mmol) in chloroform (1.2 mL) was treated with phosphorus oxychloride (600 uL) at 60° C. for 1 h 20 min. After cooling to rt, the volatile materials were removed in vacuo, and the resulting residue was dissolved in ethyl acetate. The organic solution was washed with saturated aqueous sodium bicarbonate, and the aqueous phase was back extracted with ethyl acetate. The combined organic extracts were dried over magnesium sulfate, filtered, and concentrated in vacuo to provide 2-(azidomethyl)-4-chloro-6,6-dimethyl-5,6,7,8-tetrahydroquinazoline (136 mg, 0.54 mmol, 83% yield) as an orange oil. ¹H NMR (400 MHz, CDCl₃) δ 4.47 (s, 2H), 2.94 (t, 2H), 2.55 (s, 2H), 1.68 (t, 2H), 1.05 (s, 6H); MS (EI) for C₁₁H₁₄ClN₅: 252 (MH⁺).

### Reagent Preparation 46

### 1-(4-chloro-6,6-dimethyl-5,6,7,8-tetrahydroquinazolin-2-yl)-N,N-dimethylethanamine

STEP 1: To a solution of dimethylamine (2M solution in tetrahydrofuran, 4.0 mL, 8.0 mmol) was added 2-(1-chloroethyl)-6,6-dimethyl-5,6,7,8-tetrahydroquinazolin-4-ol (synthesized according to the method of reagent preparation 18 using 2-chloropropionitrile in step 1) (50 mg, 0.21 mmol) and the reaction mixture was stirred in a sealed tube for 16 hours at 80° C. After cooling to room temperature the reaction mixture was concentrated and the residue was partitioned between brine (50 mL) and ethyl acetate (50 mL). The organic layer was separated and washed with brine (20 mL), dried over sodium sulfate, filtered and concentrated to give 2-[1-(dimethylamino)ethyl]-6,6-dimethyl-5,6,7,8-tetrahydroquinazolin-4-01 (50 mg, 96%). MS (EI) for C₁₄H₂₃N₃O: 250 (MH⁺).

STEP 2: A solution of 2-[1-(dimethylamino)ethyl]-6,6-dimethyl-5,6,7,8-tetrahydroquinazolin-4-ol (50 mg, 0.20 mmol) in a mixture of chloroform (1.5 mL) and phosphorous oxychloride (0.5 mL) was heated to reflux for 90 minutes. After cooling to room temperature the reaction mixture was concentrated and the residue was partitioned between saturated aqueous sodium bicarbonate (20 mL) and ethyl acetate (20 mL). The mixture was stirred for 15 minutes and pH was maintained above 7 by the addition of solid sodium bicarbonate. The organic layer was separated and washed with water (10 mL) and brine, dried over sodium sulfate, filtered and concentrated to give 1-(4-chloro-6,6-dimethyl-5,6,7,8-tetrahydroquinazolin-2-yl)-N,N-dimethylethanamine (46 mg, 85%). MS (EI) for C₁₄H₂₂ClN₃: 268 (MH⁺).

Using analogous synthetic techniques and substituting with alternative starting materials in step 1 the following reagent was prepared. Alternative starting materials were obtained commercially unless otherwise indicated.

4-chloro-6,6-dimethyl-2-(1-pyrrolidin-1-ylethyl)-5,6,7,8-tetrahydroquinazoline. Prepared according to the method of reagent preparation 46 by using pyrrolidine in step 1. MS (EI) for C₁₆H₂₄ClN₃: 294 (MH⁺).

### Reagent Preparation 47

### methyl 6-bromo-1H-imidazo[4,5-c]pyridin-2-ylcarbamate

A solution of 2-bromo-5-nitropyridin-4-amine (1.5 g, 6.9 mmol) in acetic acid (20 mL) was added in portions into a 75° C. suspension of iron powder (1.5 g, 27 mmol) in acetic acid (20 mL). The reaction mixture was stirred at 75° C. for 2 h, cooled to room temperature, and filtered through celite. To the filtrate was added 1,3-bis(methoxycarbonyl)-2-methyl-2-thiopseudourea (1.4 g, 6.9 mmol), and the mixture was stirred at 65° C. for 60 h. The reaction mixture was cooled to room temperature and concentrated. The solid residue was triturated with dichloromethane and dried to give the title Compound (1.8 g, quantitative yield) as an orange solid. MS (EI) for C₈H₇BrN₄O₂: 271/273 (MH⁺).

### Reagent Preparation 48

### tert-butyl 3-(bis(tert-butoxycarbonyl)amino)-5-bromo-1H-indazole-1-carboxylate

To a cooled (0° C.) solution of 5-bromo-1H-indazol-3-amine (0.30 g, 1.4 mmol), DIPEA (2.5 mL, 14 mmol) and di-tert-butyl dicarbonate (1.5 g, 7.0 mmol) in THF (15 mL) was added DMAP (0.09 g, 0.70 mmol). The reaction mixture was then stirred at ambient temperature for three hours. The resulting solution was diluted with ethyl acetate (75 mL) and washed with saturated aqueous ammonium chloride (2×50 mL). The organic layer was washed with brine, dried over magnesium sulfate, filtered and concentrated in vacuo. Purification by silica gel chromatography provided tert-butyl 3-(bis(tert-butoxycarbonyl)amino)-5-bromo-1H-indazole-1-carboxylate (0.44 g, 61%) as a waxy solid. ¹H NMR (400 MHz, CDCl₃) δ 8.04 (t, 1H), 7.68 (dd, 1H), 7.66-7.58 (m, 1H), 1.53 (s, 18H), 1.43 (s, 9H); MS (EI) for C₂₂H₃₀BrN₃O₆: 512 (MH⁺).

### Reagent Preparation 49

### 6-chloro-N-phenylpyrimidine-4-amine

STEP 1: 6-Chloropyrimidin-4-ol (500 mg, 3.85 mmol), aniline (420 μL, 4.62 mmol) and N,N-diisopropylethylamine (1 mL) in diethylene glycol dimethyl ether (5 mL) was heated to 120° C. and stirred for 8 h. The mixture was cooled to room temperature then diluted with actone:diethyl ether solution (1:1, 15 ml) to give a precipitate. The solid collected by filtration and washed with acetone then dried to afford 6-(pheylamino)pyrimidin-4-ol (255 mg, 35.5%). MS (EI) for C₁₀H₉N₃O: 188.2 (MH⁺).

STEP 2: 6-(Phenylamino)pyrimidin-4-ol (253 mg, 1.35 mmol) was dissolved in neat phosphorous oxychloride (5 mL) and stirred for 3 h at 95° C. then cooled to room temperature and concentrated. The residue was poured into an ice water slurry and extracted with dichloromethane. The extract was washed saturated aqueous sodium bicarbonate solution, dried over sodium sulfate, filtered and the solvent evaporated to afford 6-chloro-N-phenylpyrimidine-4-amine (220 mg) which was used without further purification.

Using analogous synthetic techniques and substituting with alternative starting reagents in step 1 the following reagents were prepared.

6-Chloro-N-(4-methoxyphenyl)pyrimidin-4-amine. Synthesized according to the method of reagent preparation 49 using 4-methoxyaniline in step 1.

6-Chloro-N-(3-methoxyphenyl)pyrimidin-4-amine. Synthesized according to the method of reagent preparation 49 using 3-methoxyaniline in step 1.

6-Chloro-N-(4-methoxyphenyl)-5-methylpyrimidin-4-amine. Synthesized according to the method of reagent preparation 49 using 6-chloro-5-methylpyrimidin-4-ol and 4-methoxyaniline in step 1.

6-Chloro-5-methyl-N-phenylpyrimidin-4-amine. Synthesized according to the method of reagent preparation 49 using 6-chloro-5-methylpyrimidin-4-ol and aniline in step 1.

### Reagent Preparation 50

### 5-(4,4,5,5-tetramethyl-1,3,2-dioxaborolan-2-yl)-1-methyl-1H-indazole

STEP 1: A suspension of 5-bromo-1H-indazole (200 mg, 1.02 mmol), cesium carbonate (661 mg, 2.00 mmol), and iodomethane (156 mg, 1.10 mmol) in dimethylformamide (3 mL) was stirred at room temperature for 15 h. The mixture was partitioned between 5% lithium chloride and ethyl acetate, the aqueous layer was extracted with ethyl acetate (2×), the combined organic extracts were washed with 1 N sodium hydroxide, and brine, dried over anhydrous sodium sulfate, filtered and concentrated. Column chromatography on silica (hexanes/ethyl acetate 4:1) gave 5-bromo-1-methyl-1H-indazole (150 mg, 70% yield) as an orange solid. MS (EI) for C₈H₇BrN₂: 212 (MH⁺).

STEP 2: A suspension of 5-bromo-1-methyl-1H-indazole (150 mg, 0.71 mmol), bis(pinacolato)diboron (200 mg, 0.78 mmol), potassium acetate (206 mg, 2.10 mmol), and dichloro[1,1-bis(diphenylphosphino]ferrocenepalladium (II) dichloromethane adduct (36 mg, 0.04 mmol) in dimethyl sulfoxide (4 mL) was degassed with nitrogen, and then stirred at 80° C. for 18 h. The reaction mixture was cooled to room temperature and partitioned between water and ethyl acetate. The mixture was filtered through celite and then the layers were separated. The aqueous layer was extracted with ethyl acetate (2×), the combined organic extracts were washed with brine, dried over anhydrous sodium sulfate, filtered and concentrated. Column chromatography on silica (hexanes/ethyl acetate 7:3) provided 5-(4,4,5,5-tetramethyl-1,3,2-dioxaborolan-2-yl)-1-methyl-1H-indazole (158 mg, 86% yield) as a yellow oil. MS (EI) for C₁₄H₁₉BN₂O₂: 259 (MH⁺).

### Reagent Preparation 51

### 1,1-dimethylethyl 7-bromo-2,3-dihydro-1,4-benzoxazepine-4(5H)-carboxylate

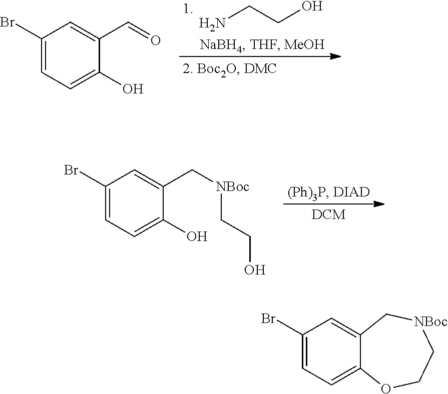

STEP 1: Commercially-available 5-bromo-2-hydroxybenzaldehyde (4.0 g, 10 mmol) and 2-aminoethanol were combined in THF/MeOH (100 mL, 10:1) and sodium borohydride (0.76 g, 2.0 mmol) was added with stirring. The resulting reaction mixture was stirred at 40° C. for 4 h, concentrated on a rotary evaporator then diluted with EtOAc (50 mL) and saturated NaHCO₃ (30 mL). To this suspension was added di-tert-butyl dicarbonate (2.83 g, 13 mmol). The mixture was stirred at rt overnight. The organic layer was washed with water, dried over anhydrous magnesium sulfate, filtered, and concentrated on a rotary evaporator. Hexane was subsequently added to the crude reaction product which resulted in the formation of a white solid. This slurry was filtered to obtain tert-butyl-5-bromo-2-hydroxybenzyl(2-hydroxyethyl)carbamate (6.8 g, 98%) as a white solid. MS (EI) for C₁₄H₂₀BrNO₄, found 346 (MH⁺).

STEP 2: tert-Butyl-5-bromo-2-hydroxybenzyl(2-hydroxyethyl)carbamate (3.46 g, 10 mmol) and triphenylphosphine (3.96 g, 15 mmol) were combined in DCM (100 mL) and diisopropyl azodicarboxylate (3.03 g, 15 mmol) was added. The resulting reaction mixture was stirred at rt for 12 h. The reaction mixture was washed with water, dried, filtered, and concentrated on a rotary evaporator. The resulting crude product was purified via silica gel chromatography eluting with 8:2 hexane/ethyl acetate to give the desired product (1.74 g, 53%) as a white solid. MS (EI) for C₁₄H₁₈BrNO₃, found 328 (MH⁺).

### Example 1

### 4-{3-[(3-fluorophenyl)methyl]-2-methylpyridin-4-yl}-7-(2-methyl-1H-benzimidazol-6-yl)-2,3,4,5-tetrahydro-1,4-benzoxazepine

STEP 1: To 5-bromo-2-methylbenzimidazole (38 g, 180 mmol) in THF (400 mL) was added di-tert-butyl dicarbonate (39 g, 189 mmol). The reaction mixture was stirred at room temperature for 24 h and then concentrated. Ethyl acetate (400 mL) was added to the residue, and the solution was washed with 10% aqueous citric acid (2×100 mL), water (100 mL), and brine (100 mL), dried over sodium sulfate, and concentrated. Column chromatography on silica (gradient 20-30% ethyl acetate in hexane) provided 1,1-dimethylethyl 6-bromo-2-methyl-1H-benzimidazole-1-carboxylate (27 g, 48% yield) as a beige solid. MS (EI) for C₁₃H₁₅BrN₂O₂: 312 (MH⁺).

STEP 2: A solution of 1,1-dimethylethyl 7-bromo-2,3-dihydro-1,4-benzoxazepine-4(5H)-carboxylate (30.0 g, 91.4 mmol) and triisopropyl borate (22.4 g, 119 mmol) in THF (300 mL) was cooled to −78° C., and a 2.5 M solution of n-butyllithium in hexanes (47.6 mL, 119 mmol) was added dropwise over 40 min at this temperature. The reaction mixture was stirred at −78° C. for an additional 30 min, then quenched by dropwise addition of 2 N hydrochloric acid (80 ml), and allowed to warm up to room temperature. Ethyl acetate (100 mL) and water (100 mL) were added, the organic layer was separated, and the aqueous layer was extracted with ethyl acetate (100 mL). The combined organic layers were washed with water, dried over sodium sulfate, and concentrated. Hexane (200 mL) was added to the residue and the mixture was stirred overnight. The precipitate was filtered, washed several times with hexane, and dried to give (4-{[(1,1-dimethylethyl)oxy]carbonyl}-2,3,4,5-tetrahydro-1,4-benzoxazepin-7-yl)boronic acid (23.4 g, 87%) as a colorless solid. MS (EI) for C₁₄H₂₀BNO₅: 294 (MH⁺).

STEP 3: A suspension of 1,1-dimethylethyl 6-bromo-2-methyl-1H-benzimidazole-1-carboxylate (11.3 g, 36 mmol), (4-{[(1,1-dimethylethyl)oxy]carbonyl}-2,3,4,5-tetrahydro-1,4-benzoxazepin-7-yl)boronic acid (11.7 g, 40 mmol), dichloro[1,1-bis-(diphenylphosphino]ferrocenepalladium (II) dichloromethane adduct (3.0 g, 10 mol %) in dioxane (115 mL) and water (28.5 mL) was degassed with nitrogen, and then diisopropylethylamine (18.6 g, 144 mmol) was added. The reaction mixture was stirred at 90° C. for 220 min, cooled to room temperature, and concentrated. Column chromatography on silica of the residue (gradient 25-30% ethyl acetate in hexane) afforded 1,1-dimethylethyl 7-(1-{[(1,1-dimethylethyl)oxy]carbonyl}-2-methyl-1H-benzimidazol-6-yl)-2,3-dihydro-1,4-benzoxazepine-4(5H)-carboxylate (13.2 g, 76% yield) as an amorphous solid. MS (EI) for C₂₇H₃₃N₃O₅: 480 (MH⁺).

STEP 4: A solution of 1,1-dimethylethyl 7-(1-{[(1,1-dimethylethyl)oxy]carbonyl}-2-methyl-1H-benzimidazol-6-yl)-2,3-dihydro-1,4-benzoxazepine-4(5H)-carboxylate (13.1 g, 27 mmol) in a mixture of methanol (20 mL) and 4 N hydrogen chloride in dioxane (30 mL) was refluxed for 15 min. After cooling to room temperature ethyl ether (100 mL) was added, and the reaction mixture was concentrated. Another portion of ethyl ether (100 mL) was added, the precipitate was filtered off, washed several times with ethyl ether, and dried to give 7-(2-methyl-1H-benzimidazol-6-yl)-2,3,4,5-tetrahydro-1,4-benzoxazepine dihydrochloride (8.9 g, 93% yield) as a light beige solid. ¹HNMR (400 MHz, CD₃OD); 7.93 (s, 1H), 7.86-7.67 (m, 4H), 7.28 (s, 1H), 4.54 (s, 2H), 4.33-4.23 (m, 2H), 3.65-3.54 (m, 2H), 2.91 (s, 3H); MS (EI) for C₁₇H₁₇N₃O: 280 (MH⁺).

STEP 5: A mixture of 4-chloro-3-[(3-fluorophenyl)methyl]-2-methylpyridine (42 mg, 0.178 mmol) synthesized according to reagent preparation 9, 7-(2-methyl-1H-benzimidazol-6-yl)-2,3,4,5-tetrahydro-1,4-benzoxazepine hydrochloride (94 mg, 0.267 mmol), and n-tributylamine (0.3 mL, 1.2 mmol) in a minimal amount of n-butanol to form a solution, was stirred in a sealed tube at 180° C. for 6 d. The reaction mixture was then cooled to ambient temperature and diluted with water (5 mL) and the aqueous layer was extracted with ethyl acetate (3×7 mL). The combined organic layers were dried over sodium sulfate, and concentrated. The residue was taken up in methanol and purified by preparative reverse phase HPLC to afford 4-{3-[(3-fluorophenyl)methyl]-2-methylpyridin-4-yl}-7-(2-methyl-1H-benzimidazol-6-yl)-2,3,4,5-tetrahydro-1,4-benzoxazepine (20 mg, 23% yield). ¹H NMR (400 MHz, CD₃OD): 8.22 (d, 1H), 7.50-7.47 (m, 3H), 7.22-7.15 (m, 3H), 7.02 (d, 1H), 6.94-6.89 (m, 1H), 6.83 (d, 1H), 6.77-6.75 (m, 4H), 4.27 (s, 2H), 4.25-4.22 (m, 2H), 4.18 (s, 2H), 3.60-3.57 (m, 2H), 2.59 (s, 3H), 2.29 (s, 3H). MS (EI) for C₃₀H₂₇FN₄O: 479.2 (MH⁺).

Using analogous synthetic techniques and substituting with alternative starting reagents in step 5 the following compounds of the invention were prepared. Protecting group introduction and removal steps were conducted as required according to literature techniques appropriate for a given protecting group (see for example: Greene and Wuts, Protective Groups in Organic Synthetic, Wiley-Interscience). Alternative starting materials were obtained commercially unless otherwise indicated.

7-(2-methyl-1H-benzimidazol-6-yl)-2,3,4,5-tetrahydro-1,4-benzoxazepine: Prepared as the dihydrochloride salt according to the method of example 1 using 4-chloroquinazoline in step 5. ¹H NMR (400 MHz, d₆-DMSO): 8.82 (s, 1H), 8.28 (d, 1H), 8.06-7.70 (m, 7H), 7.61 (dd, 1H), 7.02 (d, 1H), 5.46 (s, 2H), 4.66-4.61 (m, 2H), 4.56-4.49 (m, 2H), 2.81 (s, 3H); MS (EI) for C₂₅H₂₁N₅O: 408 (MH⁺).

7-(2-methyl-1H-benzimidazol-6-yl)-4-pyrimidin-4-yl-2,3,4,5-tetrahydro-1,4-benzoxazepine: Prepared according to the method of example 1 using 4-chloropyrimidine in step 5. ¹H NMR (400 MHz, d₆-DMSO): 12.25 (d, 1H), 8.49 (s, 1H), 8.17 (dd, 1H), 7.85 (br s, 1H), 7.74 (s, 1H), 7.58-7.35 (m, 3H), 7.08-7.00 (m, 2H), 4.87 (br s, 2H), 4.15 (br s, 4H), 3.34 (s, 3H); MS (EI) for C₂₁H₁₉N₅O: 358 (MH⁺).

4-(7-iodoquinazolin-4-yl)-7-(2-methyl-1H-benzimidazol-6-yl)-2,3,4,5-tetrahydro-1,4-benzoxazepine: Prepared according to the method of example 1 using 4-chloro-7-iodoquinazoline in step 5. ¹H NMR (400 MHz, CDCl₃): 8.67 (s, 1H), 8.31 (s, 1H), 7.71-7.65 (m, 3H), 7.62 (d, 1H), 7.53-7.49 (m, 2H), 7.44 (dd, 1H), 7.13 (d, 1H), 4.95 (s, 2H), 4.47-4.42 (m, 2H), 4.26-4.21 (m, 2H), 2.68 (s, 3H); MS (EI) for C₂₅H₂₀IN₅O: 534 (MH⁺).

7-(2-methyl-1H-benzimidazol-6-yl)-4-(2-methylquinazolin-4-yl)-2,3,4,5-tetrahydro-1,4-benzoxazepine: Prepared according to the method of example 1 using 4-chloro-2-methylquinazoline in step 5. ¹H NMR (400 MHz, d₆-DMSO): 12.26 (br s, 1H), 8.02 (d, 1H), 7.78-7.38 (m, 7H), 7.02 (d, 1H), 5.05 (br s, 2H), 4.44 (m, 2H), 4.20 (m, 2H), 3.34 (s, 3H), 2.48 (s, 3H); MS (EI) for C₂₆H₂₃N₅O: 422 (MH⁺).

ethyl 4-[7-(2-methyl-1H-benzimidazol-6-yl)-2,3-dihydro-1,4-benzoxazepin-4(5H)-yl]quinazoline-2-carboxylate: Prepared as the dihydrochloride salt according to the method of example 1 using ethyl-4-chloro-2-quinazoline carboxylate in step 5. ¹H NMR (400 MHz, d₆-DMSO): 8.24 (d, 1H), 8.04-7.94 (m, 3H), 7.89-7.81 (m, 3H), 7.74-7.68 (m, 1H), 7.60 (dd, 1H), 7.04 (d, 1H), 5.30 (s, 2H), 4.58-4.51 (m, 2H), 4.49-4.42 (m, 2H), 4.26 (q, 2H), 2.84 (s, 3H), 1.11 (t, 3H); MS (EI) for C₂₈H₂₅N₅O₃: 480 (MH⁺).

N,N-diethyl-4-[7-(2-methyl-1H-benzimidazol-6-yl)-2,3-dihydro-1,4-benzoxazepin-4(5H)-yl]quinazolin-2-amine: Prepared as the dihydrochloride salt according to the method of example 1 by sequential use of 2,4-dichloroquinazoline and N,N-diethylamine in step 5. ¹H NMR (400 MHz, d₆-DMSO): 12.1 (br s, 1H), 8.11 (br d, 1H), 8.07 (d, 1H), 7.92 (s, 1H), 7.87-7.82 (m, 3H), 7.79 (td, 1H), 7.59 (dd, 1H), 7.43 (br t, 1H), 6.99 (d, 111), 5.30 (s, 2H), 4.64-4.58 (m, 2H), 4.45 (br s, 2H), 3.58 (br s, 4H), 2.83 (s, 3H), 1.34-0.68 (m, 6H); MS (EI) for C₂₉H₃₀N₆O: 479.2 (MH⁺).

4-(2,6-diphenylpyrimidin-4-yl)-7-(2-methyl-1H-benzimidazol-6-yl)-2,3,4,5-tetrahydro-1,4-benzoxazepine: Prepared as the dihydrochloride salt according to the method of example 1 using 4-chloro-2,6-diphenylpyrimidine in step 5. ¹H NMR (400 MHz, d₆-DMSO): 8.62-8.37 (m, 2H), 8.29-8.22 (m, 2.5H), 8.11-8.00 (m, 0.5H), 7.96 (d, 1H), 7.89-7.66 (m, 2H), 7.61-7.49 (m, 7.5H), 7.42-7.32 (m, 0.5H), 7.10 (d, 1H), 5.27-5.05 (m, 2H), 4.49 (br s, 1H), 4.29 (br s, 3H), 2.83 (s, 3H); MS (EI) for C₃₃H₂₇N₅O: 510.3 (MH⁺).

7-(2-methyl-1H-benzimidazol-6-yl)-4-quinolin-4-yl-2,3,4,5-tetrahydro-1,4-benzoxazepine: Prepared as the dihydrochloride salt according to the method of example 1 using 4-chloroquinoline in step 5. ¹H NMR (400 MHz, d₆-DMSO): 8.57 (d, 1H), 8.32 (d, 1H), 8.10-7.96 (m, 4H), 7.89-7.84 (m, 2H), 7.72-7.64 (m, 2H), 7.03 (d, 1H), 6.98 (d, 1H), 5.32 (s, 2H), 4.65-4.62 (m, 2H), 4.43-4.39 (m, 2H), 2.84 (s, 3H); MS (EI) for C₂₆H₂₂N₄₀: 407 (MH⁺).

7-(2-methyl-1H-benzimidazol-6-yl)-4-[2-(trifluoromethyl)quinolin-4-yl]-2,3,4,5-tetrahydro-1,4-benzoxazepine: Prepared according to the method of example 1 using 4-chloro-2-(trifluoromethyl)quinoline in step 5. ¹H NMR (400 MHz, d₆-DMSO): 12.30 (br s, 1H), 8.05-8.15 (m, 2H), 7.75-7.85 (m, 2H), 7.40-7.66 (m, 5H), 7.28 (s, 1H), 7.05-7.10 (d, 1H), 4.85 (s, 2H), 4.40-4.45 (m, 2H), 3.98-4.05 (m, 2H), 3.36 (s, 3H). MS (EI) for C₂₇H₂₁F₃N₄₀: 475 (M+H), 473 (M−H).

7-(2-methyl-1H-benzimidazol-6-yl)-4-(2-phenylquinolin-4-yl)-2,3,4,5-tetrahydro-1,4-benzoxazepine: Prepared according to the method of example 1 using 4-chloro-2-phenylquinoline in step 5. ¹H NMR (400 MHz, d₆-DMSO): 12.35-12.21 (m, 1H), 8.17-8.10 (m, 2H), 8.07-7.98 (m, 2H), 7.92-7.65 (m, 3H), 7.62-7.41 (m, 8H), 7.07 (d, 1H), 4.80 (s, 2H), 4.40-4.33 (m, 2H), 4.05-3.95 (m, 2H), 2.52 (s, 3H). MS (EI) for C₃₂H₂₆N₄O: 483 (MH⁺).

7-(2-methyl-1H-benzimidazol-6-yl)-4-pyridin-2-yl-2,3,4,5-tetrahydro-1,4-benzoxazepine: Prepared according to the methods of example 1 using 2-chloropyridine in step 5. ¹H NMR (400 MHz, CD₃OD): 8.04-8.08 (m, 1H), 7.67 (d, 1H), 7.54-7.38 (m, 4H), 7.01 (d, 1H), 6.89 (d, 1H), 6.57-6.53 (m, 1H), 4.81 (s, 2H), 4.14 (s, 4H), 2.58 (s, 3H); MS (EI) for C₂₂H₂₀N₄O: 357 (MH⁺).

4-isoquinolin-1-yl-7-(2-methyl-1H-benzimidazol-6-yl)-2,3,4,5-tetrahydro-1,4-benzoxazepine: Prepared according to the method of example 1 using 1-chloroisoquinoline in step 5. ¹H NMR (400 MHz, CD₃OD): 8.15 (d, 1H), 8.03 (d, 1H), 7.84 (d, 1H), 7.71 (d, 1H), 7.69 (td, 1H), 7.61-7.49 (m, 5H), 7.35 (d, 1H), 7.15 (d, 1H), 4.63 (s, 2H), 4.43-4.40 (m, 2H), 3.92-3.88 (m, 2H), 2.66 (s, 3H); MS (EI) for C₂₆H₂₂N₄O: 407 (MH⁺).

7-(2-methyl-1H-benzimidazol-6-yl)-4-pyrimidin-2-yl-2,3,4,5-tetrahydro-1,4-benzoxazepine: Prepared according to the method of example 1 using 2-chloropyrimidine in step 5. ¹H NMR (400 MHz, CD₃OD): 8.22 (d, 2H), 7.56-7.53 (m, 2H), 7.42 (d, 1H), 7.32 (dt, 2H), 6.93 (d, 1H), 6.46 (t, 1H), 4.86 (s, 2H), 4.18-4.14 (m, 2H), 4.06-4.02 (m, 2H), 2.49 (s, 3H); MS (EI) for C₂₁H₁₉N₅O: 358 (MH⁺).

4-[7,8-bis(methyloxy)quinazolin-4-yl]-7-(2-methyl-1H-benzimidazol-6-yl)-2,3,4,5-tetrahydro-1,4-benzoxazepine. Synthesized according to the method of example 1 using 4-chloro-7,8-dimethoxy-quinazoline (reagent preparation 1) in step 5. ¹H NMR (400 MHz, d₆-DMSO): 8.45 (d, 1H), 7.80 (d, 1H), 7.68 (m, 1H), 7.53 (m, 2H), 7.41 (d, 1H) 7.37 (d, 1H), 7.02 (d, 1H), 5.08 (s, 2H), 4.48 (br s, 3H), 4.20 (br s, 2H), 3.93 (s, 3H), 3.86 (s, 3H), 1.78 (s, 3H); MS (EI) for C₂₇H₂₅N₅O₃: 468 (MH⁺).

7-(2-methyl-1H-benzimidazol-6-yl)-4-{8-(methyloxy)-7-[(phenylmethyl)oxy]quinazolin-4-yl}-2,3,4,5-tetrahydro-1,4-benzoxazepine. Synthesized according to the method of example 1 using 7-(benzyloxy)-4-chloro-8-methoxyquinazoline (reagent preparation 1) in step 5. ¹H NMR (400 MHz, d₆-DMSO): 8.47 (s, 1H), 7.75 (d, 1H), 7.65 (m, 2H), 7.53 (m, 4H), 7.40 (m, 4H), 7.33 (m, 1H), 7.02 (d, 2H), 5.36 (s, 2H), 5.36 (br s, 2H), 5.08 (s, 2H), 4.48 (br s, 2H), 4.18 (br s, 2H), 3.91 (s, 3H); MS (EI) for C₃₃H₂₉N₅O₃: 544 (MH⁺).

4-[6-chloro-7,8-bis(methyloxy)quinazolin-4-yl]-7-(2-methyl-1H-benzimidazol-6-yl)-2,3,4,5-tetrahydro-1,4-benzoxazepine. Synthesized according to the method of example 1 using 4,6-dichloro-7,8-dimethoxyquinazoline (reagent preparation 1) in step 5. ¹H NMR (400 MHz, d₆-DMSO): 8.55 (s, 1H), 7.83 (s, 1H), 7.74 (d, 1H), 7.53 (dd, 2H), 7.46 (m, 1), 7.04 (d, 1H), 5.02 (s, 2H), 4.48 (br s, 3H), 4.14 (br s, 2H), 4.06 (s, 3H), 4.00 (s, 3H); MS (EI) for C₂₇H₂₄ClN₅O₃:502 (MH⁺).

7-(2-methyl-1H-benzimidazol-6-yl)-4-[6,7,8-tris(methyloxy)quinazolin-4-yl]-2,3,4,5-tetrahydro-1,4-benzoxazepine. Synthesized according to the method of example 1 using 4-chloro-6,7,8-trimethoxyquinazoline in step 5. ¹H NMR (400 MHz, d₆-DMSO): 8.68 (s, 1H), 7.93 (m, 2H), 7.83 (q, 2H), 7.63 (d, 1H), 7.26 (m, 1), 7.04 (d, 1H), 5.42 (s, 2H), 4.66 (br s, 3H) 4.40 (br s, 2H), 3.96 (s, 6H), 2.82 (s, 3H), 2.51 (s, 3H); MS (EI) for C₂₈H₂₇N₅O₄: 498 (MH⁺).

7-(2-methyl-1H-benzimidazol-6-yl)-4-[6-methyl-2-(methylthio)-6,7-dihydro-5H-cyclopenta[d]pyrimidin-4-yl]-2,3,4,5-tetrahydro-1,4-benzoxazepine. Synthesized according to the method of example 1 using 4-chloro-6-methyl-2-(methylthio)-6,7-dihydro-5H-cyclopenta[d]pyrimidine (reagent preparation 4) in step 5. ¹H NMR (400 MHz, d₆-DMSO): 7.93 (br s, 2H), 7.84 (d, 1H), 7.73 (m, 2H), 7.57 (dd, 1) 7.06 (d, 1H), 5.01 (s, 2H), 4.30 (br s, 2H) 4.16 (br s, 2H), 3.21 (dd, 1H), 2.81 (s, 3H), 2.71 (dd, 1H), 2.64 (dd, 1H), 2.35 (dd, 1H); MS (EI) for C₂₆H₂₇N₅OS: 458 (MH⁺).

4-(5,7-dihydrothieno[3,4-d]pyrimidin-4-yl)-7-(2-methyl-1H-benzimidazol-6-yl)-2,3,4,5-tetrahydro-1,4-benzoxazepine. Synthesized according to the method of example 1 using 4-chloro-5,7-dihydrothieno[3,4-d]pyrimidine in step 5. ¹H NMR (400 MHz, d₆-DMSO): 8.42 (s, 1H), 7.93 (s, 1H), 7.84 (d, 1H), 7.78 (m, 2H), 7.58 (d, 1H), 7.08 (d, 1H), 5.03 (s, 2H), 4.42 (s, 2H) 4.37 (br s, 2H), 4.18 (br s, 2H), 4.07 (s, 2H), 2.80 (s, 3H); MS (EI) for C₂₃H₂₁N₅OS: 416 (MH⁺).

7-(2-methyl-1H-benzimidazol-6-yl)-4-(6-methyl-6,7-dihydro-5H-cyclopenta[d]pyrimidin-4-yl)-2,3,4,5-tetrahydro-1,4-benzoxazepine. Synthesized according to the method of example 1 using 4-chloro-6-methyl-6,7-dihydro-5H-cyclopenta[d]pyrimidine (reagent preparation 3) in step 5. ¹H NMR (400 MHz, d₆-DMSO): 8.10 (s, 1H), 7.89 (s, 1H), 7.80 (d, 1H), 7.73 (m, 2H), 7.56 (dd, 1H), 7.07 (d, 1H), 5.12 (m, 2H), 4.37 (br s, 2H) 4.26 (br s, 2H), 3.32 (dd, 1H), 3.01 (q, 1H), 2.80 (m, 1H), 2.75 (s, 3H), 1.07 (d, 3H); MS (EI) for C₂₅H₂₅N₅O: 412 (MH⁺).

4-(6-cyclopropyl-6,7-dihydro-5H-pyrrolo[3,4-d]pyrimidin-4-yl)-7-(2-methyl-1H-benzimidazol-6-yl)-2,3,4,5-tetrahydro-1,4-benzoxazepine. Synthesized according to the method of example 1 using 4-chloro-6-cyclopropyl-6,7-dihydro-5H-pyrrolo[3,4-d]pyrimidine (reagent preparation 3) in step 5. ¹H NMR (400 MHz, d₆-DMSO): 8.63 (s, 1H), 7.81 (s, 1H), 7.78 (d, 1H), 7.62 (m, 2H), 7.47 (d, 1H), 7.12 (d, 1H), 5.00 (br s, 2H), 4.90 (s, 2H), 4.51 (s, 2H), 4.33 (br s, 2H), 4.22 (br s, 2H), 3.42 (m, 1H), 2.87 (s, 3H), 0.83 (m, 4H); MS (EI) for C₂₆H₂₆N₆O: 439 (MH⁺).

7-(2-methyl-1H-benzimidazol-6-yl)-4-[6-(4-methylphenyl)-6,7-dihydro-5H-pyrrolo[3,4-d]pyrimidin-4-yl]-2,3,4,5-tetrahydro-1,4-benzoxazepine. Synthesized according to the method of example 1 using 4-chloro-6-p-tolyl-6,7-dihydro-5H-pyrrolo[3,4-d]pyrimidine (reagent preparation 3) in step 5. ¹H NMR (400 MHz, d₆-DMSO): 8.47 (m, 2H), 7.89 (d, 2H), 7.77 (m, 3H), 7.67 (d, 1H), 7.59 (d, 1H), 7.44 (d, 1H), 7.07 (d, 1H), 5.54 (s, 2H), 4.61 (m, 4H), 7.87 (s, 3H), 2.38 (m, 4H); MS (EI) for C₃₀H₂₈N₆O: 489 (MH⁺).

4-[2-chloro-6-(methyloxy)quinazolin-4-yl]-7-(2-methyl-1H-benzimidazol-6-yl)-2,3,4,5-tetrahydro-1,4-benzoxazepine. Synthesized according to the method of example 1 using 2,4-dichloro-6-methoxyquinazoline in step 5. ¹H NMR (400 MHz, d₆-DMSO): 7.97 (s, 1H), 7.93 (d, 1H), 7.86 (d, 1H), 7.83 (dd, 1H), 7.68 (d, 1H), 7.63 (dd, 1H), 7.48 (dd, 1H), 7.20 (d, 1H), 7.09 (d, 1H), 5.14 (s, 2H), 4.53 (m, 2H), 4.22 (m, 1H), 3.60 (s, 3H), 2.82 (s, 3H). MS (EI) for C₂₆H₂₂ClN₅O₂: 472 (MH⁺).

4-[6-chloro-7-(methyloxy)quinazolin-4-yl]-7-(2-methyl-1H-benzimidazol-6-yl)-2,3,4,5-tetrahydro-1,4-benzoxazepine. Synthesized according to the method of example 1 using 4,6-dichloro-7-methoxy-quinazoline (reagent preparation 1) in step 5. ¹H NMR (400 MHz, d₆-DMSO): 8.74 (s, 1H), 8.20 (s, 1H), 8.02 (s, 1H), 7.92 (d, 1H), 7.86 (s, 2H), 7.64 (dd, 1H), 7.38 (s, 1H), 7.06 (d, 1H), 5.34 (s, 2H), 4.62 (m, 2H), 4.38 (m, 2H), 4.06 (s, 3H), 2.81 (s, 3H). MS (EI) for C₂₆H₂₂ClN₅O₂: 472 (MH⁺).

7-(2-methyl-1H-benzimidazol-6-yl)-4-thieno[2,3-d]pyrimidin-4-yl-2,3,4,5-tetrahydro-1,4-benzoxazepine. Synthesized according to the method of example 1 using 4-chlorothieno[2,3-d]pyrimidine in step 5. ¹H NMR (400 MHz, d₆-DMSO): 8.38 (s, 1H), 7.78 (d, 1H), 7.72 (d, 1H), 7.66 (d, 1H), 7.62 (br s, 1H), 7.51 (br s, 0.5H), 7.49 (br s, 0.5H), 7.46 (dd, 1H), 7.36 (dd, 1H), 6.99 (d, 1H), 5.24 (s, 2H), 4.42 (m, 2H), 4.32 (m, 2H), 2.50 (s, 3H). MS (EI) for C₂₃H₂₀N₅OS: 414 (MH⁺).

7-(2-methyl-1H-benzimidazol-6-yl)-4-(5,6,7,8-tetrahydroquinazolin-4-yl)-2,3,4,5-tetrahydro-1,4-benzoxazepine. Synthesized according to the method of example 1 using 4-chloro-5,6,7,8-tetrahydroquinazoline (reagent preparation 5) in step 5. ¹H NMR (400 MHz, d₆-DMSO): 8.68 (s, 1H), 7.95 (d, 1H), 7.86 (s, 0.5H), 7.83 (s, 0.5H), 7.80-7.76 (m, 2H), 7.59 (dd, 1H), 7.05 (d, 1H), 5.12 (s, 2H), 4.48 (m, 2H), 4.21 (m, 2H), 2.82 (s, 3H), 2.76 (m, 2H), 1.78 (m, 2H), 1.62 (m, 2H). MS (EI) for C₂₅H₂₅N₅O: 412 (MH⁺).

7-(2-methyl-1H-benzimidazol-6-yl)-4-(5-methylthieno[2,3-d]pyrimidin-4-yl)-2,3,4,5-tetrahydro-1,4-benzoxazepine. Synthesized according to the method of example 1 using 4-chloro-5-methylthieno[2,3-d]pyrimidine in step 5. ¹H NMR (400 MHz, d₆-DMSO): 8.38 (s, 1H), 7.94 (d, 1H), 7.84 (d, 0.5H), 7.82 (d, 0.5H), 7.79 (d, 0.5H), 7.77 (d, 0.5H), 7.73 (d, 1H), 7.57 (dd, 1H), 7.41 (d, 1H), 7.15 (d, 1H), 4.88 (s, 2H), 4.31 (m, 2H), 3.96 (m, 2H), 2.81 (s, 3H), 2.55 (d, 3H). MS (EI) for C₂₄H₂₁N₅OS: 428 (MH⁺).

4-[7-(2-methyl-1H-benzimidazol-6-yl)-2,3-dihydro-1,4-benzoxazepin-4(5H)-yl]-6,7,8,9-tetrahydropyrimido[4,5-b]indolizine-10-carbonitrile. Synthesized according to the method of example 1 using 4-chloro-6,7,8,9-tetrahydropyrimido[4,5-b]indolizine-10-carbonitrile in step 5. ¹H NMR (400 MHz, d₆-DMSO): 8.44 (s, 1H), 7.66 (d, 1H), 7.62 (m, 1H), 7.51 (dd, 1H), 7.46 (m, 1H), 7.36 (dd, 1H), 7.05 (d, 1H), 4.40 (s, 2H), 4.37 (m, 2H), 4.31 (m, 2H), 3.84 (m, 2H), 3.14 (m, 2H), 2.50 (s, 3H). MS (EI) for C₂₈H₂₅N₇O: 476 (MH⁺).

7-(2-methyl-1H-benzimidazol-6-yl)-4-(1H-pyrazolo[3,4-d]pyrimidin-4-yl)-2,3,4,5-tetrahydro-1,4-benzoxazepine. Synthesized according to the method of example 1 using 4-chloro-1H-pyrazolo[3,4-d]pyrimidine in step 5. ¹H NMR (400 MHz, d₆-DMSO): 8.30 (br s, 1H), 7.92 (s, 1H), 7.85 (d, 1H), 7.76 (dd, 1H), 7.56 (dd, 1H), 7.06 (d, 1H), 5.31 (s, 2H), 4.42 (br s, 4H), 2.80 (s, 3H). MS (EI) for C₂₂H₁₉N₇O: 398 (MH⁺).

7-(2-methyl-1H-benzimidazol-6-yl)-4-[7-(phenylmethyl)-5,6,7,8-tetrahydropyrido[3,4-d]pyrimidin-4-yl]-2,3,4,5-tetrahydro-1,4-benzoxazepine. Synthesized according to the method of example 1 using 7-benzyl-4-chloro-5,6,7,8-tetrahydropyrido[3,4-d]pyrimidine in step 5. ¹H NMR (400 MHz, d₆-DMSO): 12.38 (br s, 1H), 8.53 (s, 1H), 7.96 (d, 1H), 7.86 (d, 0.5H), 7.84 (d, 0.5H), 7.81 (s, 1H), 7.78 (s, 0.5H), 77.75-7.70 (m, 2.5H), 7.58 (dd, 1H), 7.48 (m, 3H), 7.06 (d, 1H), 5.00 (d, 2H), 4.54 (br s, 2H), 4.44 (br s, 2H), 4.10 (m, 2H), 3.58 (m, 2H), 3.18 (m, 2H), 2.86 (m, 2H), 2.80 (s, 3H). MS (EI) for C₃₁H₃₀N₆O: 503 (MH⁺).

4-(7-fluoroquinazolin-4-yl)-7-(2-methyl-1H-benzimidazol-6-yl)-2,3,4,5-tetrahydro-1,4-benzoxazepine. Prepared according to the method of example 1 by using 4-chloro-7-fluoroquinazoline in step 5. ¹H NMR (400 MHz, methanol-d₄): 8.51 (s, 1H), 8.18 (dd, 1H), 7.68 (m, 1H), 7.63 (m, 1H), 7.54 (d, 1H), 7.51 (m, 1H), 7.46 (m, 1H), 7.41 (dd, 1H), 7.33 (m, 1H), 7.05 (d, 1H), 5.13 (s, 2H), 4.49 (m, 2H), 4.30 (m, 2H), 2.59 (s, 3H), 1.97 (s, 3H); MS (EI) for C₂₅H₂₀FN₅O: 426 (MH⁺).

7-(2-methyl-1H-benzimidazol-6-yl)-4-[8-(methyloxy)quinazolin-4-yl]-2,3,4,5-tetrahydro-1,4-benzoxazepine. Prepared according to the method of example 1 by using 4-chloro-8-methoxyquinazoline in step 5. ¹H NMR (400 MHz, methanol-d₄): 8.53 (s, 1H), 7.67 (m, 1H), 7.63 (m, 1H), 7.59 (m, 1H), 7.54 (d, 1H), 7.51 (m, 1H), 7.45 (m, 2H), 7.29 (d, 1H), 7.06 (d, 1H), 5.08 (s, 2H), 4.47 (m, 2H), 4.26 (m, 2H), 4.00 (s, 3H), 2.59 (s, 3H), 1.97 (s, 3H); MS (EI) for C₂₆H₂₃N₅O₂: 438 (MH⁺).

4-(7-chloroquinazolin-4-yl)-7-(2-methyl-1H-benzimidazol-6-yl)-2,3,4,5-tetrahydro-1,4-benzoxazepine. Prepared according to the method of example 1 by using 4,7-dichloroquinazoline in step 5. ¹H NMR (400 MHz, methanol-d₄): 8.51 (s, 1H), 8.09 (d, 1H), 7.76 (m, 1H), 7.69 (m, 1H), 7.63 (m, 1H), 7.56-7.44 (m, 4H), 7.05 (d, 1H), 5.13 (s, 2H), 4.49 (m, 2H), 4.30 (m, 2H), 2.59 (s, 3H); MS (EI) for C₂₅H₂₀ClN₅O: 442 (MH⁺).

4-(8-chloroquinazolin-4-yl)-7-(2-methyl-1H-benzimidazol-6-yl)-2,3,4,5-tetrahydro-1,4-benzoxazepine. Prepared according to the method of example 1 by using 4,8-dichloro-quinazoline in step 5. ¹H NMR (400 MHz, methanol-d₄): 8.58 (s, 1H), 8.04 (m, 1H), 7.93 (m, 1H), 7.68 (m, 1H), 7.62 (d, 1H), 7.54 (d, 1H), 7.52 (dd, 1H), 7.46 (m, 2H), 7.06 (d, 1H), 5.11 (s, 2H), 4.49 (m, 2H), 4.30 (m, 2H), 2.59 (s, 3H); MS (EI) for C₂₅H₂₀ClN₅O: 442 (MH⁺).

7-(2-methyl-1H-benzimidazol-6-yl)-4-[5-methyl-6-(phenylmethyl)pyrimidin-4-yl]-2,3,4,5-tetrahydro-1,4-benzoxazepine. Prepared as trifluoroacetate salt according to the method of example 1 by using 4-chloro-5-methyl-6-(phenylmethyl)-pyrimidine (synthesized according to reagent preparation 2) in step 5. ¹H NMR (400 MHz, methanol-d₄): 8.52 (s, 1H), 7.87 (s, 1H), 7.78 (s, 2H), 7.66 (m, 1H), 7.54 (dd, 1H), 7.33-7.16 (m, 5H), 7.06 (d, 1H), 5.12 (s, 2H), 4.46 (m, 2H), 4.24 (m, 2H), 4.19 (s, 2H), 2.87 (s, 3H), 2.33 (s, 3H); MS (EI) for C₂₉H₂₇N₅O: 462 (MH⁺).

7-(2-methyl-1H-benzimidazol-6-yl)-4-[6-(1-methylethyl)pyrimidin-4-yl]-2,3,4,5-tetrahydro-1,4-benzoxazepine. Prepared according to the method of example 1 by using 4-chloro-6-isopropyl-pyrimidine in step 5. ¹H NMR (400 MHz, methanol-4): 8.38 (s, 1H), 7.73 (d, 1H), 7.66 (m, 1H), 7.52 (d, 1H), 7.45 (m, 2H), 7.05 (d, 1H), 6.76 (s, 1H), 4.23 (br. s, 2H), 4.16 (m, 2H), 2.81 (h, 1H), 1.97 (s, 3H), 1.22 (d, 6H); MS (EI) for C₂₄H₂₅N₅O: 400 (MH⁺).

4-[5-ethyl-6-(1-methylethyl)pyrimidin-4-yl]-7-(2-methyl-1H-benzimidazol-6-yl)-2,3,4,5-tetrahydro-1,4-benzoxazepine. Prepared according to the method of example 1 by using 4-chloro-5-ethyl-6-isopropylpyrimidine (synthesized according to reagent preparation 5) in step 5. ¹H NMR (400 MHz, methanol-d₄): 8.44 (s, 1H), 7.64 (s, 1H), 7.55-7.40 (m, 4H), 7.05 (d, 1H), 4.62 (s, 2H), 4.34 (m, 2H), 3.86 (m, 2H), 2.75 (q, 2H), 2.58 (s, 3H), 1.23 (m, 9H); MS (EI) for C₂₆H₂₉N₅O: 428 (MH⁺).

7-(2-methyl-1H-benzimidazol-6-yl)-4-{6-methyl-5-[(4-methylphenyl)methyl]pyrimidin-4-yl}-2,3,4,5-tetrahydro-1,4-benzoxazepine. Prepared according to the method of example I by using 4-chloro-6-methyl-5-(4-methylbenzyl)pyrimidine (synthesized according to reagent preparation 5) in step 5. ¹H NMR (400 MHz, methanol-d₄): 8.46 (s, 1H), 7.50 (m, 2H), 7.41 (dd, 1H), 7.19 (dd, 1H), 7.03 (d, 2H), 6.98 (d, 1H), 6.93 (d, 2H), 6.64 (m, 1H), 4.50 (s, 2H), 4.29 (m, 2H), 3.95 (s, 2H), 3.89 (m, 2H), 2.60 (s, 3H), 2.24 (s, 3H), 2.21 (s, 3H); MS (EI) for C₃₀H₂₉N₅O: 476 (MH⁺).

7-(2-methyl-1H-benzimidazol-6-yl)-4-(6-methyl-5-{[4-(trifluoromethyl)phenyl]-methyl}pyrimidin-4-yl)-2,3,4,5-tetrahydro-1,4-benzoxazepine. Prepared according to the method of example 1 by using 4-chloro-6-methyl-5-(4-(trifluoromethyl)benzyl)pyrimidine (synthesized according to reagent preparation 5) in step 5. ¹H NMR (400 MHz, methanol-d₄): 8.49 (s, 1H), 7.49 (m, 4H), 7.43 (dd, 1H), 7.23 (d, 2H), 7.16 (dd, 1H), 7.00 (d, 1H), 6.68 (m, 1H), 4.51 (s, 2H), 4.31 (m, 2H), 4.08 (s, 2H), 3.88 (m, 2H), 2.60 (s, 3H), 2.20 (s, 3H); MS (EI) for C₃₀H₂₆F₃N₅O: 530 (MH⁺).

7-(2-methyl-1H-benzimidazol-6-yl)-4-[2-(methylthio)-6,7-dihydro-5H-cyclopenta[d]pyrimidin-4-yl]-2,3,4,5-tetrahydro-1,4-benzoxazepine. Synthesized according to the method of example 1 using 4-chloro-2-(methylthio)-6,7-dihydro-5H-cyclopenta[d]pyrimidine (reagent preparation 4) in step 5. ¹H NMR (400 MHz, d₆-DMSO): 7.64 (m, 2H), 7.51 (d, 1H), 7.46 (dd, 1H), 7.37 (dd, 1H), 7.01 (d, 1H), 4.94 (s, 2H), 4.24 (m, 2H), 4.20 (m, 2H), 3.00 (t, 2H), 2.64 (t, 2H), 2.42 (s, 3H), 1.92 (s and m, 5H). MS (EI) for C₂₅H₂₅N₅OS: 444 (MH⁺).

7-(2-methyl-1H-benzimidazol-6-yl)-4-{2-[(phenylmethyl)thio]-6,7-dihydro-5H-cyclopenta[d]pyrimidin-4-yl}-2,3,4,5-tetrahydro-1,4-benzoxazepine. Synthesized according to the method of example 1 using 2-(benzylthio)-4-chloro-6,7-dihydro-5H-cyclopenta[d]pyrimidine (reagent preparation 4) in step 5. ¹H NMR (400 MHz, d₆-DMSO): 7.84 (s, 1H), 7.69 (m, 3H), 7.53 (dd, 1H), 7.18 (m, 5H), 7.06 (d, 1H), 5.00 (s, 2H), 4.31 (s and m, 4H), 4.12 (m, 2H), 3.06 (t, 2H), 2.82 (s, 3H), 2.69 (t, 2H), 1.96 (m, 2H). MS (EI) for C₃₁H₂₉N₅OS: 520 (MH⁺).

4-[2-(ethylthio)-6,7-dihydro-5H-cyclopenta[d]pyrimidin-4-yl]-7-(2-methyl-1H-benzimidazol-6-yl)-2,3,4,5-tetrahydro-1,4-benzoxazepine. Synthesized according to the method of example 1 using 4-chloro-2-(ethylthio)-6,7-dihydro-5H-cyclopenta[d]pyrimidine (reagent preparation 4) in step 5. ¹H NMR (400 MHz, d₆-DMSO): 7.93 (d, 1H), 7.85 (d, 1H), 7.77 (dd, 1H), 7.72 (d, 1H), 7.56 (dd, 1H), 7.07 (d, 1H), 5.00 (s, 2H), 4.36 (m, 2H), 4.16 (m, 2H), 3.04 (m, 4H), 2.80 (s, 3H), 2.70 (t, 2H), 1.95 (m, 2H), 1.16 (t, 3H). MS (EI) for C₂₆H₂₇H₅OS: 458 (MH⁺).

4-(7,7-dimethyl-5,6,7,8-tetrahydroquinazolin-4-yl)-7-(2-methyl-1H-benzimidazol-6-yl)-2,3,4,5-tetrahydro-1,4-benzoxazepine. Synthesized according to the method of example 1 using 4-chloro-7,7-dimethyl-5,6,7,8-tetrahydroquinazoline (reagent preparation 3) in step 5. ¹H NMR (400 MHz, d₃-MeOH): 8.32 (s, 1H), 7.61 (d, 1H), 7.51 (s, 1H), 7.49 (d, 1H), 7.45 (dd, 1H), 7.39 (dd, 1H), 7.03 (d, 1H), 4.82 (s, 2H), 4.34 (t, 2H), 4.02 (t, 2H), 2.78 (t, 2H), 2.57 (s, 3H), 2.52 (s, 2H), 1.56 (t, 2H), 1.06 (s, 6H). MS (EI) for C₂₇H₂₉N₅O: 440 (MH⁺).

4-[6-bromo-7-(methyloxy)quinazolin-4-yl]-7-(2-methyl-1H-benzimidazol-6-yl)-2,3,4,5-tetrahydro-1,4-benzoxazepine. Prepared according to the method of example 1 by using 6-bromo-4-chloro-7-methoxyquinazoline (reagent preparation 1) in step 5. ¹H NMR (400 MHz, DMSO-d₆): 8.54 (s, 1H), 8.21 (s, 1H), 7.77 (m, 2H), 7.53 (m, 3H), 7.32 (s, 1H), 7.05 (d, 1H), 5.04 (s, 2H), 4.50 (m, 2H), 4.14 (m, 2H), 4.00 (s, 3H); MS (EI) for C₂₆H₂₂BrN₅O₂: 516 (MH⁺).

7-(2-methyl-1H-benzimidazol-6-yl)-4-(5-methylpyrimidin-4-yl)-2,3,4,5-tetrahydro-1,4-benzoxazepine. Prepared as trifluoroacetate salt according to the method of example 1 by using 4-chloro-5-methylpyrimidine in step 5. ¹H NMR (400 MHz, DMSO-d₆): 8.78 (br. s, 1H), 8.28 (br. s, 1H), 7.96 (s, 1H), 7.85 (d, 1H), 7.78 (m 2H), 7.60 (m, 1H), 7.07 (d, 1H), 5.17 (s, 2H), 4.47 (m, 2H), 4.28 (m, 2H), 2.82 (s, 3H), 2.41 (s, 31-1); MS (EI) for C₂₂H₂₁N₅O: 372 (MH⁺).

4-(5,6-dimethylpyrimidin-4-yl)-7-(2-methyl-1H-benzimidazol-6-yl)-2,3,4,5-tetrahydro-1,4-benzoxazepine. Prepared according to the method of example 1 by using 4-chloro-5,6-dimethylpyrimidine in step 5. ¹H NMR (400 MHz, DMSO-d₆): 12.00 (br. s, 1H), 8.35 (s, 1H), 7.65 (m, 1H), 7.61 (m, 1H), 7.50 (m, 2H), 7.38 (dd, 1H), 7.03 (d, 1H), 4.62 (s, 2H), 4.32 (m, 2H), 3.81 (m, 2H), 2.33 (s, 3H), 2.20 (s, 3H), 1.91 (s, 3H); MS (EI) for C₂₃H₂₃N₅O: 386 (MH⁺).

7-(2-methyl-1H-benzimidazol-6-yl)-4-[6-methyl-5-(phenylmethyl)pyrimidin-4-yl]-2,3,4,5-tetrahydro-1,4-benzoxazepine. Prepared according to the method of example 1 by using 5-benzyl-4-chloro-6-methylpyrimidine (reagent preparation 5) in step 5. ¹H NMR (400 MHz, DMSO-d₆): 12.26 (br. s, 1H), 8.51 (s, 1H), 7.50 (br. s, 2H), 7.46 (dd, 1H), 7.36-7.24 (m, 3H), 7.18 (d, 1H), 7.11 (d, 2H), 7.00 (d, 1H), 6.81 (s, 1H), 4.47 (s, 2H), 4.27 (m, 2H), 4.03 (s, 2H), 3.79 (m, 2H), 2.17 (s, 3H), 1.91 (s, 3H); MS (EI) for C₂₉H₂₇N₅O: 462 (MH⁺).

7-(2-methyl-1H-benzimidazol-6-yl)-4-[6-methyl-5-(1-methylethyl)pyrimidin-4-yl]-2,3,4,5-tetrahydro-1,4-benzoxazepine. Prepared according to the method of example 1 by using 4-chloro-6-methyl-5-(1-methylethyl)-pyrimidine (reagent preparation 5) in step 5. ¹H NMR (400 MHz, DMSO-d₆): 8.43 (s, 1H), 7.63 (s, 1H), 7.52 (m, 3H), 7.37 (dd, 1H), 7.06 (d, 1H), 4.42 (s, 2H), 4.30 (m, 2H), 3.68 (m, 2H), 3.31 (h, 1H), 1.89 (s, 3H), 1.33 (d, 6H); MS (EI) for C₂₅H₂₇N₅O: 414 (MH⁺).

7-(2-methyl-1H-benzimidazol-6-yl)-4-[6-methyl-5-(2-methylpropyl)pyrimidin-4-yl]-2,3,4,5-tetrahydro-1,4-benzoxazepine. Prepared according to the method of example 1 by using 4-chloro-5-isobutyl-6-methylpyrimidine (reagent preparation 5) in step 5. ¹H NMR (400 MHz, DMSO-d₆): 12.30 (br. s, 1H), 8.37 (s, 1H), 7.62 (m, 2H), 7.49 (m, 2H), 7.37 (d, 1H), 7.01 (d, 1H), 4.59 (s, 2H), 4.33 (m, 2H), 3.75 (m, 2H), 2.61 (d, 2H), 2.37 (s, 3H), 1.68 (m, 1H), 0.53 (d, 6H); MS (EI) for C₂₆H₂₉N₅O: 428 (MH⁺).

4-{5-[(3-fluorophenyl)methyl]-6-methylpyrimidin-4-yl}-7-(2-methyl-1H-benzimidazol-6-yl)-2,3,4,5-tetrahydro-1,4-benzoxazepine. Prepared according to the method of example 1 by using 4-chloro-5-(3-fluorobenzyl)-6-methylpyrimidine (reagent preparation 5) in step 5. ¹H NMR (400 MHz, DMSO-d₆): 12.35 (br. s, 1H), 8.51 (s, 1H), 7.49 (m, 3H), 7.35 (m, 1H), 7.19 (d, 1H), 7.10 (m, 1H), 7.00 (d, 1H), 6.92 (m, 3H), 4.49 (s, 2H), 4.27 (m, 2H), 4.05 (s, 2H), 3.78 (m, 2H), 2.52 (s, 3H), 2.17 (s, 3H); MS (EI) for C₂₉H₂₆FN₅O: 480 (MH⁺).

4-{5-[(3-chlorophenyl)methyl]-6-methylpyrimidin-4-yl}-7-(2-methyl-1H-benzimidazol-6-yl)-2,3,4,5-tetrahydro-1,4-benzoxazepine. Prepared according to the method of example 1 by using 4-chloro-5-(3-chlorobenzyl)-6-methylpyrimidine (reagent preparation 5) in step 5. ¹H NMR (400 MHz, DMSO-d₆): 12.31 (br. s, 1H), 8.51 (s, 1H), 7.50 (m, 2H), 7.47 (dd, 1H), 7.33 (m, 2H), 7.19 (dd, 1H), 7.14 (s, 1H), 7.04 (m, 1H), 7.00 (d, 1H), 6.95 (d, 11-1), 4.51 (s, 2H), 4.28 (m, 2H), 4.05 (s, 2H), 3.78 (m, 2H), 2.52 (s, 3H), 2.17 (s, 3H); MS (EI) for C₂₉H₂₆ClN₅O: 496 (MH⁺)

7-(2-methyl-1H-benzimidazol-6-yl)-4-[6-methyl-5-(1-phenylethyl)pyrimidin-4-yl]-2,3,4,5-tetrahydro-1,4-benzoxazepine. Prepared according to the method of example 1 by using 4-chloro-6-methyl-5(1-phenylethyl)pyrimidine (reagent preparation 5) in step 5. ¹H NMR (400 MHz, DMSO-d₆): 8.60 (s, 1H), 7.76 (d, 1H), 7.72 (s, 1H), 7.54 (dd, 1H), 7.49 (d, 1H), 7.34-7.18 (m, 5H), 7.06 (d, 1H), 7.01 (s, 1H), 4.61-4.44 (m, 3H), 4.37 (m, 1H), 4.28 (m, 1H), 3.87 (m, 1H), 3.77 (m, 1H), 2.77 (s, 3H), 2.11 (s, 3H), 1.66 (d, 3H); MS (EI) for C₃₀H₂₉N₅O: 476 (MH⁺).

7-(2-methyl-1H-benzimidazol-6-yl)-4-(6-methyl-5-{[3-(methyloxy)phenyl]methyl}pyrimidin-4-yl)-2,3,4,5-tetrahydro-1,4-benzoxazepine. Prepared according to the method of example 1 by using 4-chloro-5-(3-methoxybenzyl)-6-methylpyrimidine (reagent preparation 5) in step 5. ¹H NMR (400 MHz, DMSO-d₆): 12.27 (br. s, 1H), 8.50 (s, 1H), 7.47 (m, 3H), 7.26 (m, 1H), 7.16 (d, 1H), 7.00 (d, 1H), 6.85 (m, 2H), 6.65 (m, 2H), 4.50 (s, 2H), 4.28 (m, 2H), 3.99 (s, 2H), 3.79 (m, 2H), 3.55 (s, 3H), 2.51 (s, 3H), 2.17 (s, 3H); MS (EI) for C₃₀H₂₉N₅O₂: 492 (MH⁺).

7-(2-methyl-1H-benzimidazol-6-yl)-4-{6-methyl-5-[(3-methylphenyl)methyl]pyrimidin-4-yl}-2,3,4,5-tetrahydro-1,4-benzoxazepine. Prepared according to the method of example 1 by using 4-chloro-6-methyl-5-(3-methylbenzyl)pyrimidine (reagent preparation 5) in step 5. ¹H NMR (400 MHz, DMSO-d₆): 12.21 (d, 1H), 8.48 (s, 1H), 7.50 (m, 1H), 7.43 (dd, 1H), 7.38 (d, 1H), 7.21 (m, 1H), 7.13 (m, 1H), 7.04 (m, 1H), 6.98 (dd, 1H), 6.89 (m, 2H), 6.72 (dd, 1H), 4.45 (s, 2H), 4.26 (m, 2H), 3.93 (m, 2H), 3.77 (m, 2H), 2.49 (s, 3H), 2.14 (s, 3H), 2.04 (d, 3H); MS (EI) for C₃₀H₂₉N₅O: 476 (MH⁺).

4-{5-[(3-chloro-5-fluorophenyl)methyl]-6-methylpyrimidin-4-yl}-7-(2-methyl-1H-benzimidazol-6-yl)-2,3,4,5-tetrahydro-1,4-benzoxazepine. Prepared according to the method of example 1 by using 4-chloro-5-(3-chloro-5-fluorobenzyl)-6-methylpyrimidine (reagent preparation 5) in step 5. ¹H NMR (400 MHz, methanol-d₄): 8.49 (s, 1H), 7.52 (m, 2H), 7.44 (dd, 1H), 7.27 (d, 1H), 7.06 (m, 1H), 7.00 (d, 1H), 6.88 (m, 2H), 6.76 (d, 1H), 4.54 (s, 2H), 4.30 (m, 2H), 4.04 (s, 2H), 3.88 (m, 2H), 2.59 (s, 3H), 2.22 (s, 3H); MS (EI) for C₂₉H₂₅ClFN₅O: 514 (MH⁺).

4-{5-[(3,4-difluorophenyl)methyl]-6-methylpyrimidin-4-yl}-7-(2-methyl-1H-benzimidazol-6-yl)-2,3,4,5-tetrahydro-1,4-benzoxazepine. Prepared according to the method of example 1 by using 4-chloro-5-(3,4-difluorobenzyl)-6-methylpyrimidine (reagent preparation 5) in step 5. ¹H NMR (400 MHz, DMSO-d₆): 12.22 (br. s, 1H), 8.50 (s, 1H), 7.62-7.43 (m, 3H), 7.32 (m, 1H), 7.18 (m, 1H), 7.14 (m, 1H), 6.99 (m, 2H), 6.88 (m, 1H), 4.50 (s, 2H), 4.27 (m, 2H), 4.01 (s, 2H), 3.76 (m, 2H), 2.51 (s, 3H), 2.17 (s, 3H); MS (EI) for C₂₉H₂₅F₂N₅O: 498 (MH⁺).

4-{5-[(4-fluorophenyl)methyl]-2,6-dimethylpyrimidin-4-yl}-7-(2-methyl-1H-benzimidazol-6-yl)-2,3,4,5-tetrahydro-1,4-benzoxazepine. Prepared according to the method of example 1 by using 4-chloro-5-(4-fluorobenzyl)-2,6-dimethylpyrimidine (reagent preparation 8) in step 5. ¹H NMR (400 MHz, DMSO-d₆): 12.25 (br. s, 1H), 7.61-7.43 (m, 3H), 7.18 (dd, 2H), 7.11 (d, 1H), 7.00 (d, 1H), 6.94 (d, 1H), 4.44 (s, 2H), 4.21 (m, 2H), 3.97 (s, 2H), 3.75 (m, 2H), 2.51 (s, 3H), 2.41 (s, 3H), 2.11 (s, 3H); MS (EI) for C₃₀H₂₈FN₅O: 494 (MH⁺).

4-(7-chloro-6-iodoquinazolin-4-yl)-7-(2-methyl-1H-benzimidazol-6-yl)-2,3,4,5-tetrahydro-1,4-benzoxazepine. Prepared according to the method of example 1 by using 4,7-dichloro-6-iodoquinazoline (reagent preparation 1) in step 5. ¹H NMR (400 MHz, DMSO-d₆): 12.26 (br. s, 1H), 8.59 (s, 1H), 8.49 (s, 1H), 7.99 (s, 1H), 7.77 (m, 2H), 7.58-7.47 (m, 3H), 7.04 (d, 1H), 5.08 (s, 2H), 4.52 (m, 2H), 4.16 (m, 2H); MS (EI) for C₂₅H₁₉Cl₁N₅O: 568 (MH⁺).

7-(2-methyl-1H-benzimidazol-6-yl)-4-[7-(methylsulfonyl)quinazolin-4-yl]-2,3,4,5-tetrahydro-1,4-benzoxazepine. Prepared according to the method of example 1 by using 4-chloro-7-(methylsulfonyl)quinazoline (reagent preparation 1) in step 5. ¹H NMR (400 MHz, methanol-4): 8.62 (s, 1H), 831 (m, 2H), 7.94 (dd, 1H), 7.68 (s, 1H), 7.65 (d, 1H), 7.55 (d, 1H), 7.51 (dd, 1H), 7.46 (dd, 1H), 7.04 (d, 1H), 5.19 (s, 2H), 4.51 (m, 2H), 4.35 (m, 2H), 3.21 (s, 3H), 2.59 (s, 3H); MS (EI) for C₂₆H₂₃N₅O₃S: 486 (MH⁺).

4-(6-ethyl-5-methylpyrimidin-4-yl)-7-(2-methyl-1H-benzimidazol-6-yl)-2,3,4,5-tetrahydro-1,4-benzoxazepine. Prepared according to the method of example 1 by using 4-chloro-6-ethyl-5-methyl-pyrimidine (reagent preparation 5) in step 5. ¹H NMR (400 MHz, methanol-d₄): 8.36 (s, 1H), 7.63 (s, 1H), 7.52 (m, 2H), 7.46 (dd, 1H), 7.42 (dd, 1H), 7.04 (d, 1H), 4.68 (s, 2H), 4.33 (m, 2H), 3.92 (m, 2H), 2.73 (q, 2H), 2.58 (s, 3H), 2.30 (s, 3H), 1.22 (t, 3H); MS (EI) for C₂₄H₂₅N₅O: 400 (MH⁺).

4-(5,6-diethylpyrimidin-4-yl)-7-(2-methyl-1H-benzimidazol-6-yl)-2,3,4,5-tetrahydro-1,4-benzoxazepine. Prepared according to the method of example 1 by using 4-chloro-5,6-diethyl-pyrimidine (reagent preparation 5) in step 5. ¹H NMR (400 MHz, methanol-d₄): 8.39 (s, 1H), 7.66 (s, 1H), 7.54 (m, 2H), 7.46 (m, 2H), 7.04 (d, 1H), 4.70 (s, 2H), 4.35 (m, 2H), 3.92 (m, 2H), 2.77 (m, 4H), 2.60 (s, 3H), 1.24 (t, 3H), 1.20 (t, 3H); MS (EI) for C₂₅H₂₈N₅O: 414 (MH⁺).

4-[6-ethyl-5-(phenylmethyl)pyrimidin-4-yl]-7-(2-methyl-1H-benzimidazol-6-yl)-2,3,4,5-tetrahydro-1,4-benzoxazepine. Prepared according to the method of example 1 by using 5-benzyl-4-chloro-6-ethylpyrimidine (reagent preparation 5) in step 5. ¹H NMR (400 MHz, methanol-d₄): 8.52 (s, 1H), 7.50 (d, 1H), 7.466 (s, 1H), 7.42 (dd, 1H), 7.24 (m, 4H), 7.08 (m, 2H), 6.99 (d, 1H), 6.70 (d, 1H), 4.50 (s, 2H), 4.28 (m, 2H), 4.04 (s, 2H), 3.89 (m, 2H), 2.61 (s, 3H), 2.51 (q, 2H), 1.10 (t, 3H); MS (EI) for C₃₀H₃₀N₅O: 476 (MH⁺).

5-methyl-6-[7-(2-methyl-1H-benzimidazol-6-yl)-2,3-dihydro-1,4-benzoxazepin-4(5H)-yl]-N-phenylpyrimidin-4-amine. Prepared as acetate salt according to the method of example 1 by using 6-chloro-5-methyl-N-phenylpyrimidin-4-amine (reagent preparation 6) in step 5. ¹H NMR (400 MHz, methanol-d₄): 8.07 (s, 1H), 7.64 (s, 1H), 7.53-7.38 (m, 6H), 7.27 (m, 2H), 7.07 (d, 1H), 7.03 (m, 1H), 4.57 (s, 2H), 4.32 (m, 2H), 3.82 (m, 2H), 2.58 (s, 3H), 2.19 (s, 3H), 1.97 (s, 3H); MS (EI) for C₂₈H₂₆N₆O: 463 (MH⁺).

4-[7-(2-methyl-1H-benzimidazol-6-yl)-2,3-dihydro-1,4-benzoxazepin-4(5H)-yl]pyrimidin-2-amine. Prepared as acetate salt according to the method of example 1 by using 2-amino-4-chloropyrimidine in step 5. ¹H NMR (400 MHz, methanol-d₄): 7.72-7.65 (m, 3H), 7.52 (d, 1H), 7.46 (m, 2H), 7.06 (d, 1H), 6.35 (d, 1H), 4.17 (m, 4H), 2.58 (s, 3H), 1.94 (s, 3H); MS (EI) for C₂₁H₂₀N₆O: 373 (MH⁺).

7-(2-methyl-1H-benzimidazol-6-yl)-4-(6-methyl-5-{[2-(methyloxy)phenyl]methyl}pyrimidin-4-yl)-2,3,4,5-tetrahydro-1,4-benzoxazepine. Prepared according to the method of example 1 by using 4-chloro-5-(2-methoxybenzyl)-6-methylpyrimidine (reagent preparation 5) in step 5. ¹H NMR (400 MHz, methanol-d₄): 8.48 (s, 1H), 7.52 (d, 1H), 7.43 (dd, 1H), 7.39 (s, 1H), 7.31 (m, 1H), 7.20 (dd, 1H), 6.98 (d, 2H), 6.95 (m, 1H), 6.86 (d, 1H), 6.60 (d, 1H), 6.50 (d, 1H), 4.42 (s, 2H), 4.29 (m, 2H), 3.90 (m, 2H), 3.77 (s, 2H), 3.22 (s, 3H), 2.61 (s, 3H), 2.15 (s, 3H); MS (EI) for C₃₀H₂₉N₅O₂: 492 (MH⁺).

7-(2-methyl-1H-benzimidazol-6-yl)-4-{6-methyl-5-[(2-methylphenyl)methyl]pyrimidin-4-yl}-2,3,4,5-tetrahydro-1,4-benzoxazepine. Prepared according to the method of example 1 by using 4-chloro-6-methyl-5-(2-methylbenzyl)pyrimidine (reagent preparation 5) in step 5. ¹H NMR (400 MHz, methanol-d₄): 8.50 (s, 1H), 7.51 (d, 1H), 7.45 (dd, 1H), 7.38 (s, 1H), 7.18 (m, 3H), 7.00 (d, 1H), 6.90 (d, 1H), 6.77 (d, 1H), 6.27 (d, 1H), 4.41 (s, 2H), 4.35 (m, 2H), 3.90 (m, 2H), 3.70 (s, 2H), 3.62 (s, 3H), 2.16 (s, 3H), 1.78 (s, 3H); MS (EI) for C₃₀H₂₉N₅O: 476 (MH⁺).

7-(2-methyl-1H-benzimidazol-6-yl)-4-[2-methyl-3-(phenylmethyl)pyridin-4-yl]-2,3,4,5-tetrahydro-1,4-benzoxazepine. Prepared as acetate salt according to the method of example 1 by using 3-benzyl-4-chloro-2-methylpyridine (reagent preparation 9) in step 5. ¹H NMR (400 MHz, methanol-d₄): 8.20 (d, 1H), 7.49 (d, 1H), 7.44 (m, 2H), 7.20 (m, 4H), 7.15 (d, 1H), 7.02 (m, 3H), 6.66 (d, 1H), 4.30 (s, 2H), 4.25 (m, 2H), 4.16 (s, 2H), 3.62 (m, 2H), 2.60 (s, 3H), 2.29 (s, 3H), 1.96 (s, 3H); MS (EI) for C₃₀H₂₈N₄O: 461 (MH⁺).

4-{3-[(4-fluorophenyl)methyl]-2-methylpyridin-4-yl}-7-(2-methyl-1H-benzimidazol-6-yl)-2,3,4,5-tetrahydro-1,4-benzoxazepine. Prepared according to the method of example 1 by using 4-chloro-3(4-fluorobenzyl)-2-methylpyridine (reagent preparation 9) in step 5. ¹H NMR (400 MHz, methanol-d₄): 8.21 (d, 1H), 7.50 (m, 2H), 7.44 (dd, 1H), 7.17 (dd, 1H), 7.13 (d, 1H), 7.03 (d, 1H), 6.99 (m, 2H), 6.88 (m, 2H), 6.68 (s, 1H), 4.22 (m, 4H), 4.13 (s, 2H), 3.56 (m, 2H), 2.60 (s, 3H), 2.28 (s, 3H); MS (O) for C₃₀H₂₇FN₄O: 479 (MH⁺).

4-[6,7-bis(methyloxy)quinazolin-4-yl]-7-(2-methyl-1H-benzimidazol-6-yl)-2,3,4,5-tetrahydro-1,4-benzoxazepine. Prepared according to the method of example 1 by using 4-chloro-6,7-dimethoxyquinazoline in step 5. ¹H NMR (400 MHz, Methanol-D₄): 8.50 (1H), 7.74 (br, 1H), 7.70 (br, 1H), 7.62 to 7.53 (m, 3H), 7.17 (s, 1H), 7.14 (s, 1H), 7.09 (d, 1H), 5.14 (s, 2H), 4.60 (m, 2H), 4.27 (m, 2H), 3.96 (s, 3H), 3.54 (s, 3H), 2.66 (s, 3H), MS (EI) for C₂₇H₂₅N₅O₃: 468 (MH⁺).

7-(2-methyl-1H-benzimidazol-6-yl)-4-[7-(methyloxy)quinazolin-4-yl]-2,3,4,5-tetrahydro-1,4-benzoxazepine. Prepared according to the method of example 1 by using 4-chloro-7-methoxyquinazoline in step 5. ¹H NMR (400 MHz, DMSO-D₆): 8.48, (s, 1H), 7.99 (d, 1H), 7.71 to 7.67 (m, 2H), 7.62 to 7.50 (m, 2H), 7.45 (d, 1H), 7.18 (d, 1H), 7.13 (dd, 1H), 7.03 (d, 1H), 5.10 (s, 2H), 4.49 (m, 2H), 4.20 (m, 2H), 3.91 (s, 3H), 2.54 (s, 3H), MS (EI) for C₂₆H₂₃N₅O₂: 438 (MH⁺).

7-(2-methyl-1H-benzimidazol-6-yl)-4-{6-(methyloxy)-7-[(phenylmethyl)oxy]quinazolin-4-yl}-2,3,4,5-tetrahydro-1,4-benzoxazepine. Prepared according to the method of example 1 by using 7-(benzyloxy)-4-chloroquinazoline in step 5. ¹H NMR (400 MHz, Methanol-D₄): 8.43, (s, 1H), 7.99 (d, 2H), 7.54 to 7.49 (m, 2H), 7.45 to 7.41 (m, 3H), 7.38 to 7.29 (m, 3H), 7.18 (d, 1H), 7.08 (dd, 2H), 5.20 (s, 2H), 4.97 (s, 2H), 4.52 (m, 2H), 4.10 (m, 2H), 3.56 (s, 3H), 2.59 (s, 3H), MS (EI) for C₃₃H₂₉N₅O₃: 544 (MH⁺).

7-(2-methyl-1H-benzimidazol-6-yl)-4-[6-(methyloxy)quinazolin-4-yl]-2,3,4,5-tetrahydro-1,4-benzoxazepine. Prepared according to the method of example 1 by using 4-chloro-6-methoxyquinazoline in step 5. ¹H NMR (400 MHz, Methanol-D₄): 8.47, (s, 1H), 7.70 (d, 1H), 7.65 (dd, 2H), 7.54 to 749 (m, 2H), 7.44 (dd, 1H), 7.40 (dd, 1H), 7.15 (d, 1H), 7.07 (d, 1H), 5.00 (s, 2H), 4.52 (m, 2H), 4.19 (m, 2H), 3.49 (s, 3H), 2.60 (s, 3H), MS (EI) for C₂₆H₂₃N₅O₂: 438 (MH⁺).

4-(6-bromoquinazolin-4-yl)-7-(2-methyl-1H-benzimidazol-6-yl)-2,3,4,5-tetrahydro-1,4-benzoxazepine. Prepared according to the method of example 1 by using 6-bromo-4-chloroquinazoline in step 5. ¹H NMR (400 MHz, DMSO-D₆): 8.61, (s, 1H), 8.17 (d, 1H), 7.95 (dd, 1H), 7.77 to 7.73 (m, 3H), 7.44 (dd, 2H), 7.49 (br, 1H), 7.04 (d, 1H), 5.06 (s, 2H), 4.52 (m, 2H), 4.16 (m, 2H), 2.51 (s, 3H), MS (EI) for C₂₅H₂₀BrN₅O: 486 (MH⁺).

4-(6-chloroquinazolin-4-yl)-7-(2-methyl-1H-benzimidazol-6-yl)-2,3,4,5-tetrahydro-1,4-benzoxazepine. Prepared according to the method of example 1 by using 4,6-dichloroquinazoline in step 5. ¹H NMR (400 MHz, DMSO-D₆): 8.59, (s, 1H), 8.04 (d, 1H), 7.87 to 7.81 (m, 4H), 7.77 (d, 1H), 7.55 (dd, 1H), 7.47 (br, 1H), 7.04 (d, 1H), 5.08 (s, 2H), 4.52 (m, 2H), 4.18 (m, 2H), 2.51 (s, 3H), MS (EI) for C₂₅H₂₃N₅O₂: 441 (MH⁺).

7-(2-methyl-1H-benzimidazol-6-yl)-4-(8-methylquinazolin-4-yl)-2,3,4,5-tetrahydro-1,4-benzoxazepine. Prepared according to the method of example 1 by using 4-chloro-8-methylquinazoline in step 5. ¹H NMR (400 MHz, Methanol-D₄): 8.56, (s, 1H), 7.90 (d, 1H), 7.65 (dd, 2H), 7.57 to 7.37 (m, 6H), 7.05 (d, 1H), 5.04 (s, 2H), 4.45 (m, 2H), 4.24 (m, 2H), 2.63 (s, 3H), 2.58 (s, 3H), MS (EI) for C₂₆H₂₃N₅O: 422 (MH⁺).

7-(2-methyl-1H-benzimidazol-6-yl)-4-(6-methylquinazolin-4-yl)-2,3,4,5-tetrahydro-1,4-benzoxazepine. Prepared according to the method of example 1 by using 4-chloro-6-methylquinazoline in step 5. ¹H NMR (400 MHz, Methanol-D₄): 8.45, (s, 1H), 7.85 (br, 1H), 7.72 to 7.64 (m, 2H), 7.56 to 7.47 (m, 4H), 7.07 (d, 1H), 5.05 (s, 2H), 4.50 (m, 2H), 4.23 (m, 2H), 2.59 (s, 3H), 2.38 (s, 3H), MS (EI) for C₂₆H₂₃N₅O: 422 (MH⁺).

4-(6-iodoquinazolin-4-yl)-7-(2-methyl-1H-benzimidazol-6-yl)-2,3,4,5-tetrahydro-1,4-benzoxazepine. Prepared according to the method of example 1 by using 4-chloro-6-iodoquinazoline in step 5. ¹H NMR (400 MHz, DMSO-D₆): 8.61, (s, 1H), 8.33 (d, 1H), 8.08 (d, 1H), 7.83 to 7.74 (m, 2H), 7.59 to 7.47 (m, 4H), 7.05 (d, 1H), 5.05 (s, 2H), 4.51 (m, 2H), 4.15 (m, 2H), 2.53 (s, 3H), MS (EI) for C₂₅H₂₀IN₅O: 534 (MH⁺).

4-(6-fluoroquinazolin-4-yl)-7-(2-methyl-1H-benzimidazol-6-yl)-2,3,4,5-tetrahydro-1,4-benzoxazepine. Prepared according to the method of example 1 by using 4-chloro-6-fluoroquinazoline in step 5. ¹H NMR (400 MHz, DMSO-D₆): 8.56, (s, 1H), 7.88 (dd, 1H), 7.80 to 7.70 (m, 4H), 7.58 to 7.42 (m, 2H), 7.47 (dd, 1H), 7.05 (d, 1H), 5.10 (s, 2H), 4.53 (m, 2H), 4.20 (m, 2H), 2.54 (s, 3H), MS (EI) for C₂₅H₂₀FN₅O: 426 (MH⁺).

4-(6,7-difluoroquinazolin-4-yl)-7-(2-methyl-1H-benzimidazol-6-yl)-2,3,4,5-tetrahydro-1,4-benzoxazepine. Prepared according to the method of example 1 by using 4-chloro-6,7-difluoroquinazoline in step 5. ¹H NMR (400 MHz, DMSO-D₆): 8.56, (s, 1H), 8.01 (dd, 1H), 7.87 to 7.70 (m, 3H), 7.55 to 7.51 (m, 2H), 7.45 (d, 1H), 7.02 (d, 1H), 5.11 (s, 2H), 4.53 (m, 2H), 4.20 (m, 2H), 2.53 (s, 3H), MS (EI) for C₂₅H₁₉F₂N₅: 444 (MH⁺).

4-(6-bromo-7-chloroquinazolin-4-yl)-7-(2-methyl-1H-benzimidazol-6-yl)-2,3,4,5-tetrahydro-1,4-benzoxazepine. Prepared according to the method of example 1 by using 6-bromo-4,7-dichloroquinazoline in step 5. ¹H NMR (400 MHz, DMSO-D₆): 8.60, (s, 1H), 8.33 (s, 1H), 8.06 (s, 1H), 7.79 (br, 2H), 7.59 to 7.50 (m, 2H), 7.00 (d, 1H), 5.10 (s, 2H), 4.529 (m, 2H), 4.18 (m, 2H), 2.53 (s, 3H), MS (EI) for C₂₅H₁₉BrClN₅O: 522 (MH⁺).

4-[7-bromo-8-(methyloxy)quinazolin-4-yl]-7-(2-methyl-1H-benzimidazol-6-yl)-2,3,4,5-tetrahydro-1,4-benzoxazepine. Prepared according to the method of example 1 by using 7-bromo-4-chloro-8-methoxyquinazoline (reagent preparation 1) in step 5. ¹H NMR (400 MHz, DMSO-D₆): 8.56, (s, 1H), 7.72 to 7.63 (m, 3H), 7.54 to 7.45 (m, 2H), 7.41 (dd, 1H), 7.00 (d, 1H), 5.11 (s, 2H), 4.49 (m, 2H), 4.21 (m, 2H), 4.02 (s, 3H), 2.51 (s, 3H), MS (EI) for C₂₆H₂₂BrN₅O₂: 516 (MH⁺).

4-[7-bromo-6-(methyloxy)quinazolin-4-yl]-7-(2-methyl-1H-benzimidazol-6-yl)-2,3,4,5-tetrahydro-1,4-benzoxazepine. Prepared according to the method of example 1 by 7-bromo-4-chloro-6-methoxyquinazoline in step 5. ¹H NMR (400 MHz, DMSO-D₆): 8.52, (s, 1H), 8.09 (s, 1H), 7.83 (br, 1H), 7.55 (dd, 2H), 7.40 (d, 1H), 7.18 (s, 1H), 7.05 (d, 1H), 5.08 (s, 2H), 4.55 (m, 2H), 4.13 (m, 2H), 3.60 (s, 3H), 2.51 (s, 3H), MS (EI) for C₂₆H₂₂BrN₅O₂: 516 (MH⁺).

4-[6-iodo-7-(methyloxy)quinazolin-4-yl]-7-(2-methyl-1H-benzimidazol-6-yl)-2,3,4,5-tetrahydro-1,4-benzoxazepine. Prepared according to the method of example 1 by using 4-chloro-6-iodo-7-methoxyquinazoline in step 5. ¹H NMR (400 MHz, DMSO-D₆): 8.53, (s, 1H), 8.40 (s, 1H), 7.78 (br, 1H), 7.73 (br, 1H), 7.54 (dd, 1H), 7.51 (s, 1H) 7.21 (s, 1H), 7.05 (d, 1H), 5.02 (s, 2H), 4.50 (m, 2H), 4.13 (m, 2H), 3.97 (s, 3H), 2.51 (s, 3H), MS (EI) for C₂₆H₂₂IN₅O₂: 564 (MH⁺).

7-(2-methyl-1H-benzimidazol-6-yl)-4-(7-methyl-5,6,7,8-tetrahydroquinazolin-4-yl)-2,3,4,5-tetrahydro-1,4-benzoxazepine. Prepared according to the method of example 1 by using 4-chloro-7-methyl-5,6,7,8-tetrahydroquinazoline (reagent preparation 8) in step 5. ¹H NMR (400 MHz, DMSO-D₆): 8.34 (s, 1H), 7.65 to 7.59 (m, 2H), 7.51 to 7.46 (m, 2H), 7.36 (dd, 1H), 7.02 (d, 1H), 4.70 (dd, 2H), 4.39 (m, 1H), 4.24 (m, 1H), 3.94 to 3.82 (m, 2H), 2.94 to 2.80 (m, 2H), 2.57 to 2.46 (m, 1H), 2.51 (s, 3H), 2.26 (dd, 1H), 1.92 (m, 1H), 1.82 (dd, 1H), 1.15 (m, 1H), 1.04 (d, 3H), MS (EI) for C₂₆H₂₇N₅O₂: 426 (MH⁺).

4(6,6-dimethyl-5,6,7,8-tetrahydroquinazolin-4-yl)-7-(2-methyl-1H-benzimidazol-6-yl)-2,3,4,5-tetrahydro-1,4-benzoxazepine. Prepared according to the method of example 1 by using 4-chloro-6,6-dimethyl-5,6,7,8-tetrahydroquinazoline (reagent preparation 3) in step 5. ¹H NMR (400 MHz, Methano-D₄): 8.35 (s, 1H), 7.65 (br, 1H), 7.54 to 7.42 (m, 4H), 7.03 (d, 1H), 4.70 (dd, 2H), 4.35 (m, 2H), 3.95 (m, 2H), 2.78 (t, 2H), 2.58 (s, 3H), 2.50 (s, 2H), 1.68 (t, 2H), 0.90 (s, 6H), MS (EI) for C₂₇H₂₉N₅O: 440 (MH⁺).

4-(6-ethyl-5,6,7,8-tetrahydroquinazolin-4-yl)-7-(2-methyl-1H-benzimidazol-6-yl)-2,3,4,5-tetrahydro-1,4-benzoxazepine. Prepared according to the method of example 1 by using 4-chloro-6-ethyl-5,6,7,8-tetrahydroquinazoline (reagent preparation 8) in step 5. ¹H NMR (400 MHz, Methanol-D₄): 8.33 (s, 1H), 7.65 (br, 1H), 7.51 (dd, 2H), 7.45 to 7.39 (m, 2H), 7.03 (d, 1H), 4.69 (dd, 2H), 4.45 (m, 1H), 4.24 (m, 1H), 4.07 (m, 1H), 3.83 (m, 1H), 2.85 (dd, 1H), 2.72 (m, 1H), 2.58 (s, 3H), 2.54 (br, 1H), 2.40 (m, 1H), 1.93 (m, 1H), 1.49 to 1.26 (m, 3H), 0.75 (t, 3H), MS (EI) for C₂₇H₂₉NO: 440 (MH⁺).

4-{5-[(4-fluorophenyl)methyl]-6-methylpyrimidin-4-yl}-7-(2-methy 1-1H-benzimidazol-6-yl)-2,3,4,5-tetrahydro-1,4-benzoxazepine. Prepared according to the method of example 1 by using 4-chloro-5-(4-fluorobenzyl)-6-methylpyrimidine (reagent preparation 5) in step 5. ¹H NMR (400 MHz, methanol-d₄): 8.47 (s, 1H), 7.54-7.50 (m, 2H), 7.43 (d, 1H), 7.24 (d, 1H), 7.11-7.05 (m, 2H), 7.02-6.92 (m, 3H), 6.76 (s, 1H), 4.53 (s, 2H), 4.30 (t, 2H), 4.01 (s, 2H), 3.90 (t, 2H), 2.60 (s, 3H), 2.22 (s, 3H); MS (EI) for C₂₉H₂₆FN₅O: 480 (MH⁺).

4-(7-bromoquinazolin-4-yl)-7-(2-methyl-1H-benzimidazol-6-yl)-2,3,4,5-tetrahydro-1,4-benzoxazepine. Prepared according to the method of example 1 by using 7-bromo-4-chloroquinazoline in step 5. ¹H NMR (400 MHz, d₆-DMSO): 12.26 (br s, 1H), 8.52 (s, 1H), 8.02-7.96 (m, 2H), 7.71-7.61 (m, 3H), 7.54-7.48 (m, 2H), 7.41 (d, 1H), 6.99 (d, 1H), 4.49 (t, 2H), 4.22 (t, 2H), 2.51 (s, 3H); MS (EI) for C₂₅H₂₀BrN₅O: 487 (MH⁺).

4-(8-bromoquinazolin-4-yl)-7-(2-methyl-1H-benzimidazol-6-yl)-2,3,4,5-tetrahydro-1,4-benzoxazepine. Prepared as the acetate salt according to the method of example 1 by using 8-bromo-4-chloroquinazoline in step 5. ¹H NMR (400 MHz, d₆-DMSO): 12.26 (br s, 1H), 8.61 (s, 1H), 8.18 (d, 2H), 8.06 (d, 1H), 7.71-7.66 (m, 2H), 7.52 (d, 2H), 7.45-7.39 (m, 2H), 7.01 (d, 1H), 5.13 (s, 2H), 4.49 (t, 2H), 4.23 (t, 2H), 2.51 (s, 3H); MS (EI) for C₂₅H₂₀BrN₅O: 487 (MH⁺).

7-(2-methyl-1H-benzimidazol-6-yl)-4-(7-methylquinazolin-4-yl)-2,3,4,5-tetrahydro-1,4-benzoxazepine. Prepared as the acetate salt according to the method of example 1 by using 4-chloro-7-methylquinazoline in step 5. ¹H NMR (400 MHz, d₆-DMSO): 12.26 (br s, 1H), 8.50 (s, 1H), 7.95 (d, 1H), 7.67 (s, 2H), 7.58 (s, 1H), 7.54-7.49 (m, 2H), 7.40 (d, 1H), 7.33 (d, 1H), 7.01 (d, 1H), 5.09 (s, 2H), 4.49 (t, 2H), 4.19 (t, 2H), 2.51 (s, 3H), 2.48 (s, 3H); MS (EI) for C₂₆H₂₃N₅O: 422 (MH⁺).

4-(8-fluoroquinazolin-4-yl)-7-(2-methyl-1H-benzimidazol-6-yl)-2,3,4,5-tetrahydro-1,4-benzoxazepine. Prepared according to the method of example 1 by using 4-chloro-8-fluoroquinazoline in step 5. ¹H NMR (400 MHz, d₆-DMSO): 8.60 (s, 1H), 7.98 (s, 1H), 7.93 (d, 1H), 7.89-7.81 (m, 3H), 7.74 (t, 1H), 7.61 (d, 1H), 7.54 (m, 1H), 7.05 (d, 1H), 5.23 (s, 2H), 4.56 (t, 2H), 4.31 (t, 2H), 2.82 (s, 3H); MS (EI) for C₂₅H₂₀FN₅O: 426 (MH⁺).

7-(2-methyl-1H-benzimidazol-6-yl)-4-[8-(trifluoromethyl)quinazolin-4-yl]-2,3,4,5-tetrahydro-1,4-benzoxazepine. Prepared according to the method of example 1 by using 4-chloro-8-(trifluoromethyl)quinazoline in step 5. ¹H NMR (400 MHz, d₆-DMSO): 8.61 (s, 1H), 8.33 (d, 1H), 8.21 (d, 1H), 7.80-7.74 (m, 2H), 7.67-7.54 (m, 2H), 7.22 (s, 1H), 7.10 (s, 1H), 7.03-6.96 (m, 1H), 5.18 (s, 2H), 4.52 (t, 2H), 4.27 (t, 2H), 2.63 (s, 3H); MS (EI) for C₂₆H₂₀F₃N₅O: 476 (MH⁺).

4-(6,8-dichloroquinazolin-4-yl)-7-(2-methyl-1H-benzimidazol-6-yl)-2,3,4,5-tetrahydro-1,4-benzoxazepine. Prepared according to the method of example 1 by using 4,6,8-trichloroquinazoline in step 5. ¹H NMR (400 MHz, d₆-DMSO): 8.64 (s, 1H), 8.19 (s, 1H), 8.00 (s, 1H), 7.80-7.76 (m, 2H), 7.59-7.43 (m, 3H), 7.04 (d, 1H), 5.09 (s, 2H), 4.55 (t, 2H), 4.20 (t, 2H), 2.50 (s, 3H); MS (EI) for C₂₅H₁₉Cl₂N₅O: 477 (MH⁺).

7-(2-methyl-1H-benzimidazol-6-yl)-4-[7-(trifluoromethyl)quinazolin-4-yl]-2,3,4,5-tetrahydro-1,4-benzoxazepine. Prepared according to the method of example 1 by using 4-chloro-7-(trifluoromethyl)quinazoline in step 5. ¹H NMR (400 MHz, d₆-DMSO): 12.00 (br. s, 1H), 8.62 (s, 1H), 8.29 (d, 1H), 8.11 (s, 1H), 7.88 (s, 1H), 7.82-7.71 (m, 3H), 7.67 (d, 1H), 7.57 (d, 1H), 7.03 (d, 1H), 5.21 (s, 2H), 4.53 (t, 2H), 4.29 (t, 2H), 2.70 (s, 3H); MS (EI) for C₂₆H₂₀F₃N₅O: 476 (MH⁺).

7-(2-methyl-1H-benzimidazol-6-yl)-4-[6-(methylsulfonyl)quinazolin-4-yl]-2,3,4,5-tetrahydro-1,4-benzoxazepine. Prepared as the acetate salt according to the method of example 1 by using 4-chloro-6-(methylsulfonyl)quinazoline (reagent preparation 1) in step 5. ¹H NMR (400 MHz, methanol-d₄): 8.68 (d, 1H), 8.63 (s, 1H), 8.24 (d, 1H), 7.95 (d, 1H), 7.77 (s, 1H), 7.72 (s, 1H), 7.54-7.51 (m, 3H), 7.05 (d, 1H), 5.20 (s, 2H), 4.53 (t, 2H), 4.37 (t, 2H), 3.06 (s, 3H), 2.59 (s, 3H); MS (EI) for C₂₆H₂₃N₅O₃S: 486 (MH⁺).

7-(2-methyl-1H-benzimidazol-6-yl)-4-[5-methyl-6-(1-methylethyl)pyrimidin-4-yl]-2,3,4,5-tetrahydro-1,4-benzoxazepine. Prepared according to the method of example 1 by using 4-chloro-6-isopropyl-5-methylpyrimidine (reagent preparation 5) in step 5. ¹H NMR (400 MHz, methanol-d₄): 8.31 (s, 1H), 7.54 (s, 1H), 7.44-7.40 (m, 2H), 7.39-7.31 (m, 2H), 6.95 (d, 1H), 4.55 (s, 2H), 4.24 (t, 2H), 3.80 (t, 2H), 3.21 (m, 1H), 2.49 (s, 3H), 2.20 (s, 3H), 1.12 (d, 6H); MS (EI) for C₂₅H₂₇N₅O: 414 (MH⁺).

4-(5-ethyl-6-methylpyrimidin-4-yl)-7-(2-methyl-1H-benzimidazol-6-yl)-2,3,4,5-tetrahydro-1,4-benzoxazepine. Prepared as the trifluoroacetate salt according to the method of example 1 by using 4-chloro-5-ethyl-6-methylpyrimidine in step 5. ¹H NMR (400 MHz, methanol-d₄): 8.50 (s, 1H), 7.90 (s, 1H), 7.83-7.77 (m, 2H), 7.72 (s, 1H), 7.55 (d, 1H), 7.05 (d, 1H), 5.17 (s, 2H), 4.50 (t, 2H), 4.26 (t, 2H), 2.88 (s, 3H), 2.80 (q, 2H), 2.53 (s, 3H), 1.23 (t, 3H); MS (EI) for C₂₄H₂₅N₅O: 400 (MH⁺).

4-[5-(cyclopropylmethyl)-6-methylpyrimidin-4-yl]-7-(2-methyl-1H-benzimidazol-6-yl)-2,3,4,5-tetrahydro-1,4-benzoxazepine. Prepared as described in example 1 using 4-chloro-5-(cyclopropylmethyl)-6-methylpyrimidine (reagent preparation 5) in step 5. ¹H NMR (400 MHz, methanol-d₄): 8.36 (s, 1H), 7.64 (s, 1H), 7.57-7.41 (m, 4H), 7.04 (d, 1H), 4.65 (s, 2H), 4.35 (t, 1H), 3.89 (t, 2H), 2.71 (d, 2H), 2.58 (s, 3H), 2.49 (s, 3H), 0.86 (m, 1H), 0.36 (m, 2H), 0.04 (m, 2H); MS (EI) for C₂₆H₂₇N₅O: 426 (MH⁺).

4-{5-[(4-chlorophenyl)methyl]-6-methylpyrimidin-4-yl}-7-(2-methyl-1H-benzimidazol-6-yl)-2,3,4,5-tetrahydro-1,4-benzoxazepine. Prepared according to the method of example 1 using 4-chloro-5-(4-chlorobenzyl)-6-methylpyrimidine (reagent preparation 5) in step 5. ¹H NMR (400 MHz, methanol-d₄): 8.47 (s, 1H), 7.55-7.51 (m, 2H), 7.43 (d, 1H), 7.21-7.17 (m, 3H), 7.06-6.98 (m, 3H), 6.71 (s, 1H), 4.51 (s, 1H), 4.31 (t, 2H), 3.99 (s, 2H), 3.88 (t, 2H), 2.60 (s, 3H), 2.21 (s, 3H); MS (EI) for C₂₉H₂₆ClN₅O: 497 (MH⁺).

4-{5-[(3,5-difluorophenyl)methyl]-6-methylpyrimidin-4-yl}-7-(2-methyl-1H-benzimidazol-6-yl)-2,3,4,5-tetrahydro-1,4-benzoxazepine. Prepared according to the method of example 1 using 4-chloro-5-(3,5-difluorobenzyl)-6-methylpyrimidine (reagent preparation 5) in step 5. ¹H NMR (400 MHz, methanol-d₄): 8.49 (s, 1H), 7.52-7.49 (m, 2H), 7.45 (d, 1H), 7.28 (d, 1H), 7.00 (d, 1H), 6.90 (s, 1H), 6.83 (t, 1H), 6.68 (d, 2H), 4.55 (s, 2H), 4.30 (t, 2H), 4.07 (s, 2H), 3.89 (t, 2H); MS (EI) for C₂₉H₂₅F₂N₅O: 498 (MH⁺).

7-(2-methyl-1H-benzimidazol-6-yl)-4-(6-methyl-5-{[3-(trifluoromethyl)phenyl]methyl}pyrimidin-4-yl)-2,3,4,5-tetrahydro-1,4-benzoxazepine. Prepared according to the method of example 1 using 4-chloro-6-methyl-5-(3-(trifluoromethyl)benzyl)pyrimidine (reagent preparation 5) in step 5. ¹H NMR (400 MHz, methanol-d₄): 8.49 (s, 1H), 7.55-7.48 (m, 3H), 7.45-7.37 (m, 2H), 7.35 (s, 1H), 7.30 (d, 1H), 7.24 (d, 1H), 7.00 (d, 1H), 6.88 (s, 1H), 4.57 (s, 2H), 4.28 (t, 2H), 4.15 (s, 2H), 3.88 (t, 2H), 2.60 (s, 3H), 2.21 (s, 3H); MS (EI) for C₃₀H₂₆F₃N₅O: 530 (MH⁺).

2-chloro-N,N-dimethyl-5-({4-methyl-6-[7-(2-methyl-1H-benzimidazol-6-yl)-2,3-dihydro-1,4-benzoxazepin-4(5H)-yl]pyrimidin-5-yl}methyl)aniline. Prepared as the trifluoroacetate salt according to the method of example 1 using 4-chloro-5-(4-chloro-3-fluorobenzyl)-6-methylpyrimidine (reagent preparation 5) in step 5, subsequent side reaction displacement of the 3 fluoro by dimethyl amine yielded the title compound. ¹H NMR (400 MHz, methanol-d₄): 8.64 (s, 1H), 7.80-7.77 (m, 2H), 7.67 (d, 1H), 7.53 (d, 1H), 7.29 (d, 1H), 7.16 (s, 1H), 7.05 (d, 1H), 6.91 (s, 1H), 6.73 (d, 1H), 5.03 (s, 2H), 4.39 (t, 2H), 4.18 (t, 2H), 4.05 (s, 2H), 2.88 (s, 3H), 2.63 (s, 6H), 2.32 (s, 3H); MS (EI) for C₃₁H₃₁ClN₆O: 540 (MH⁺).

4-{5-[1-(3-fluorophenyl)ethyl]-6-methylpyrimidin-4-yl}-7-(2-methyl-1H-benzimidazol-6-yl)-2,3,4,5-tetrahydro-1,4-benzoxazepine. Prepared as the acetate salt according to the method of example 1 using 4-chloro-5-(1-(3-fluorophenyl)ethyl)-6-methylpyrimidine (reagent preparation 5) in step 5. ¹H NMR (400 MHz, methanol-d₄): 8.51 (s, 1H), 7.50-7.43 (m, 3H), 7.32-7.25 (m, 1H), 7.17 (d, 1H), 7.05-6.95 (m, 4H), 6.83 (s, 1H), 4.60 (q, 1H), 4.43-4.35 (m, 2H), 4.26-4.19 (m, 1H), 3.99-3.91 (m, 1H), 3.82-3.74 (m, 1H), 2.60 (s, 3H), 2.14 (s, 3H), 1.63 (d, 3H); MS (EI) for C₃₀H₂₈FN₅O: 494 (MH⁺).

4-(8-bromo-6-methylquinazolin-4-yl)-7-(2-methyl-1H-benzimidazol-6-yl)-2,3,4,5-tetrahydro-1,4-benzoxazepine. Prepared according to the method of example 1 by using 8-bromo-4-chloro-6-methylquinazoline (reagent preparation 1) in step 5. ¹H NMR (400 MHz, d₆-DMSO): 12.25 (br. s, 1H), 8.59 (s, 1H), 8.05 (s, 1H), 7.80-7.76 (m, 2H), 7.65 (s, 1H), 7.55 (d, 1H), 7.47-7.41 (m, 2H), 7.03 (d, 1H), 5.07 (s, 2H), 4.51 (t, 2H), 4.17 (t, 2H), 2.50 (s, 3H), 2.35 (s, 3H); MS (EI) for C₂₆H₂₂BrN₅O: 501 (MH⁺).

1-{4-ethyl-5-methyl-6-[7-(2-methyl-1H-benzimidazol-5-yl)-2,3-dihydro-1,4-benzoxazepin-4(5H)-yl]pyrimidin-2-yl}-N,N-dimethylmethanamine. Prepared according to the method of example 1 by using 1-(4-chloro-6-ethyl-5-methylpyrimidin-2-yl)-N,N-dimethylmethanamine (reagent preparation 17) in step 5. ¹H NMR (400 MHz, DMSO-d6) δ 7.74-7.30 (m, 5H), 7.00 (d, 1H), 4.60 (s, 2H), 4.32-4.25 (m, 2H), 3.84-3.76 (m, 2H), 3.37 (s, 2H), 2.64 (q, 2H), 2.49 (s, 3H), 2.20 (s, 3H), 2.14 (s, 6H), 1.15 (t, 3H); MS (EI) for C₂₇H₃₂N₆O: 457 (MH⁺).

N,N-dimethyl-1-{4-[7-(2-methyl-1H-benzimidazol-5-yl)-2,3-dihydro-1,4-benzoxazepin-4(5H)-yl]-5-(1-methylethyl)pyrimidin-2-yl}methanamine. Prepared according to the method of example 1 by using 1-(4-chloro-5-isopropylpyrimidin-2-yl)-N,N-dimethylmethanamine (reagent preparation 17) in step 5. ¹H NMR (400 MHz, DMSO-d6) δ 8.35 (s, 1H), 7.63 (s, 1H), 7.57 (d, 1H), 7.51 (d, 1H), 7.46 (dd, 1H), 7.37 (dd, 1H), 7.00 (d, 1H), 4.63 (s, 2H), 4.35-4.29 (m, 2H), 3.86-3.80 (m, 2H), 3.38 (s, 2H), 3.16-3.05 (m, 2H), 2.50 (s, 3H), 2.11 (s, 6H), 1.88 (s, 3H), 1.25 (d, 6H); MS (EI) for C₂₇H₃₂N₆O: 457 (MH⁺).

1-{5-ethyl-4-methyl-6-[7-(2-methyl-1H-benzimidazol-5-yl)-2,3-dihydro-1,4-benzoxazepin-4(5H)-yl]pyrimidin-2-yl}-N,N-dimethylmethanamine. Prepared as an acetate salt by the method of example 1 using 1-(4-chloro-5-ethyl-6-methylpyrimidin-2-yl)-N,N-dimethylmethanamine (reagent preparation 17) in step 5. ¹H NMR (400 MHz, DMSO-d6) δ 7.69 (br s, 1H), 7.60 (d, 1H), 7.53 (br s, 1H), 7.46 (dd, 1H), 7.40-7.34 (m, 1H), 7.00 (d, 1H), 4.61 (s, 2H), 4.34-4.28 (m, 2H), 3.83-3.76 (m, 2H), 3.42-3.38 (m, 2H), 2.62 (q, 2H), 2.50 (s, 3H), 2.36 (s, 3H), 2.12 (s, 6H), 1.89 (s, 3H), 1.16 (t, 3H); MS (EI) for C₂₇H₃₂N₆O: 457 (MH⁺).

4-[6,6-dimethyl-2-({[2-(methyloxy)ethyl]oxy}methyl)-5,6,7,8-tetrahydroquinazolin-4-yl]-7-(2-methyl-1H-benzimidazol-6-yl)-2,3,4,5-tetrahydro-1,4-benzoxazepine. Prepared according to the method of example 1 by using 4-chloro-6,6-dimethyl-2-({[2-(methyloxy)ethyl]oxy}methyl)-5,6,7,8-tetrahydroquinazoline (reagent preparation 17) in step 5. ¹H NMR (400 MHz, d₆-DMSO): 7.64 (d, 2H), 7.47 (dd, 2H), 7.38 (dd, 1H), 7.01 (d, 1H), 4.63 (s, 2H), 4.29 (m, 2H), 3.86 (m, 2H), 3.56 (m, 2H), 3.38 (m, 2H), 3.32 (brs, 2H), 3.17 (s, 3H), 2.70 (m, 2H), 2.49 (s, 3H), 2.47 (s, 2H) 1.60 (m, 2H), 0.87 (s, 6H); MS (EI) for C₃₁H₃₇N₅O₃: 528 (MH⁺).

N-([6,6-dimethyl-4-[7-(2-methyl-1H-benzimidazol-6-yl)-2,3-dihydro-1,4-benzoxazepin-4(5H)-yl]-5,6,7,8-tetrahydroquinazolin-2-yl]methyl)-2-(methyloxy)ethanamine. Prepared according to the method of example 1 by using N-[(4-chloro-6,6-dimethyl-5,6,7,8-tetrahydroquinazolin-2-yl)methyl]-2-(methyloxy)ethanamine (reagent preparation 17) in step 5. ¹H NMR (400 MHz, d₆-DMSO): 9.50 (brs, 1H), 7.98 (s, 2H), 7.82 (s, 2H), 7.61 (dd, 1H), 7.05 (d, 1H), 5.08 (brs, 1H), 4.49 (s, 2H), 4.28 (s, 2H), 4.12 (s, 2H), 3.56 (s, 1H), 3.20 (s, 3H), 3.12 (m, 2H), 2.86 (s, 3H), 2.78 (m, 2H), 2.54 (s, 2H), 2.51 (s, 3H), 1.60 (m, 2H), 0.87 (s, 6H); MS (EI) for C₃₁H₃₈N₆O₂: 527 (MH⁺).

4-[6,6-dimethyl-2-(pyrrolidin-1-ylmethyl)-5,6,7,8-tetrahydroquinazolin-4-yl]-7-(2-methyl-1H-benzimidazol-5-yl)-2,3,4,5-tetrahydro-1,4-benzoxazepine. Prepared according to the method of example 1 by using 4-chloro-6,6-dimethyl-2-(pyrrolidin-1-ylmethyl)-5,6,7,8-tetrahydroquinazoline (reagent preparation 17) in step 5. ¹H NMR (400 MHz, Methanol-d₄): 7.70 (br, 1H), 7.63 (br, 1H), 7.56 to 7.44 (m, 3H), 7.02 (d, 1H), 4.83 (s, 2H), 4.39 (m, 2H), 4.18 (s, 2H), 4.01 (m, 2H), 3.17 (m, 4H), 2.88 (t, 2H), 2.59 (s, 3H), 2.51 (s, 2H), 1.88 (m, 4H), 1.69 (t, 2H), 0.92 (s, 6H); MS (EI) for C₃₂H₃₈N₆O: 523 (MH⁺).

4-{6,6-dimethyl-2-[(2R)-pyrrolidin-2-yl]-5,6,7,8-tetrahydroquinazolin-4-yl}-7-(2-methyl-1H-benzimidazol-5-yl)-2,3,4,5-tetrahydro-1,4-benzoxazepine. Prepared according to the method of example 1 by using phenylmethyl (2R)-2-(4-chloro-6,6-dimethyl-5,6,7,8-tetrahydroquinazolin-2-yl)pyrrolidine-1-carboxylate (reagent preparation 18) in step 5 followed by Cbz group deprotection. ¹H NMR (400 MHz, Methanol-d₄): 7.70 (s, 1H), 7.63 (s, 1H), 7.54 (d, 1H), 7.47 (m, 2H), 7.00 (d, 1H), 4.84 (s, 2H), 4.46 (m, 1H), 4.37 (m, 2H), 4.01 (m, 2H), 3.13 (m, 2H), 2.80 (t, 2H), 2.59 (s, 3H), 2.52 (dd, 2H), 2.32 (m, 1H), 1.94 (m, 1H), 1.85 (m, 1H), 1.76 (m, 1H), 1.70 (t, 2H), 0.93 (d, 6H); MS (EI) for C₃₁H₃₆N₆O: 509 (MH⁺).

{5-[(4-fluorophenyl)methyl]-4-methyl-6-[7-(2-methyl-1H-benzimidazol-5-yl)-2,3-dihydro-1,4-benzoxazepin-4(5H)-yl]pyrimidin-2-yl}methyl acetate. Prepared according to the method of example 1 by using {4-chloro-5-[(4-fluorophenyl)methyl]-6-methylpyrimidin-2-yl}methyl acetate (reagent preparation 17) in step 5. ¹H NMR (400 MHz, Methanol-d₄): 7.57 (s, 1H), 7.50 (s, 1H), 7.43 (d, 1H), 7.25 (d, 1H), 7.09 (m, 2H), 7.03 to 6.93 (m, 3H), 6.83 (d, 1H), 5.07 (s (2H), 4.54 (s, 2H), 4.25 (m, 2H), 4.01 (s, 2H), 3.87 (m, 2H), 2.62 (s, 3H), 2.22 (s, 3H), 2.10 (s, 3H); MS (EI) for C₃₂H₃₀FN₅O₃: 552 (MH⁺).

{5-[(4-fluorophenyl)methyl]-4-methyl-6-[7-(2-methyl-1H-benzimidazol-5-yl)-2,3-dihydro-1,4-benzoxazepin-4(5H)-yl]pyrimidin-2-yl}methanol. Prepared according to the method of example 1 by using {4-chloro-5-[(4-fluorophenyl)methyl]-6-methylpyrimidin-2-yl}methyl acetate (reagent preparation 17) in step 5, followed by acetate hydrolysis using standard techniques. ¹H NMR (400 MHz, Methanol-d₄): 7.57 to 7.53 (m, 2H), 7.41 (d, 1H), 7.27 (d, 1H), 7.09 (m, 2H), 7.03 to 6.94 (m, 3H), 6.89 (d, 1H), 4.57 (s (2H), 4.55 (s, 2H), 4.26 (m, 2H), 4.00 (s, 2H), 3.91 (m, 2H), 2.59 (s, 3H), 2.21 (s, 3H); MS (EI) for C₃₀H₂₈FN₅O₂: 510 (MH⁺).

N-({6,6-dimethyl-4-[7-(2-methyl-1H-benzimidazol-6-yl)-2,3-dihydro-1,4-benzoxazepin-4(5H)-yl]-5,6,7,8-tetrahydroquinazolin-2-yl}methyl)-2-fluoroethanamine. Prepared as diacetate salt according to the method of example 1 by using phenylmethyl[(4-chloro-6,6-dimethyl-5,6,7,8-tetrahydroquinazolin-2-yl)methyl](2-fluoroethyl)carbamate (reagent preparation 17) in step 5 followed by Cbz deprotecttion. ¹H NMR (400 MHz, methanol-d₄): 7.66 (d, 1H), 7.59 (d, 1H), 7.52 (d, 1H), 7.46 (m, 2H), 7.02 (d, 1H), 4.78 (s, 2H), 4.44 (m, 2H), 4.35 (m, 2H), 4.01 (m, 2H), 3.86 (s, 2H), 2.93 (m, 2H), 2.79 (m, 2H), 2.58 (s, 3H), 2.52 (s, 2H), 1.95 (s, 6H), 1.68 (m, 2H), 0.92 (s, 6H); MS (EI) for C₃₀H₃₅FN₆O: 515 (MH⁺).

N-({6,6-dimethyl-4-[7-(2-methyl-1H-benzimidazol-6-yl)-2,3-dihydro-1,4-benzoxazepin-4(5H)-yl]-5,6,7,8-tetrahydroquinazolin-2-yl}methyl)cyclopropanamine. Prepared as acetate salt according to the method of example 1 by using N-[(4-chloro-6,6-dimethyl-5,6,7,8-tetrahydroquinazolin-2-yl)methyl]cyclopropanamine (reagent preparation 17) in step 5. ¹H NMR (400 MHz, methanol-d₄): 7.68 (d, 1H), 7.64 (d, 1H), 7.52 (d, 1H), 7.47 (m, 2H), 7.00 (d, 1H), 4.81 (s, 2H), 4.33 (m, 2H), 4.02 (m, 2H), 3.83 (s, 2H), 2.78 (m, 2H), 2.58 (s, 3H), 2.52 (s, 2H), 2.17 (m, 1H), 1.95 (s, 3H), 1.67 (m, 2H), 0.93 (s, 6H), 0.33 (m, 4H); MS (EI) for C₃₁H₃₆N₆O: 509 (MH⁺).

N-({6,6-dimethyl-4-[7-(2-methyl-1H-benzimidazol-6-yl)-2,3-dihydro-1,4-benzoxazepin-4(5H)-yl]-5,6,7,8-tetrahydroquinazolin-2-yl}methyl)ethanamine. Prepared as acetate salt according to the method of example 1 by using benzyl (4-chloro-6,6-dimethyl-5,6,7,8-tetrahydroquinazolin-2-yl)methyl(ethyl)carbamate (reagent preparation 17) in step 5 followed by Cbz deprotection. ¹H NMR (400 MHz, methanol-d₄): 7.66 (s, 1H), 7.60 (d, 1H), 7.53-7.43 (m, 3H), 7.03 (d, 1H), 4.81 (s, 2H), 4.36 (m, 2H), 4.02 (m, 2H), 3.98 (s, 2H), 2.88 (q, 2H), 2.80 (t, 2H), 2.58 (s, 3H), 2.52 (s, 2H), 1.90 (s, 3H), 1.69 (t, 2H), 1.10 (t, 3H), 0.92 (s, 6H); MS (EI) for C₃₀H₃₆N₆O: 497 (MH⁺).

1-{5-(cyclopropylmethyl)-4-methyl-6-[7-(2-methyl-1H-benzimidazol-6-yl)-2,3-dihydro-1,4-benzoxazepin-4(5H)-yl]pyrimidin-2-yl}-N,N-dimethylmethanamine. Prepared as dihydrochloride salt according to the method of example 1 by using 1-(4-chloro-5-(cyclopropylmethyl)-6-methylpyrimidin-2-yl)-N,N-dimethylmethanamine (reagent preparation 17) in step 5. ¹H NMR (400 MHz, methanol-d₄): 8.00 (s, 1H), 7.88 (m, 2H), 7.81 (d, 1H), 7.60 (dd, 1H), 7.08 (d, 1H), 5.20 (s, 2H), 4.58 (m, 2H), 4.54 (s, 2H), 4.24 (m, 2H), 2.92 (s, 6H), 2.81 (s, 3H), 2.74 (d, 2H), 2.59 (s, 3H), 0.86 (m, 1H), 0.44 (m, 2H), 0.02 (m, 211); MS (EI) for C₂₉H₃₄N₆O: 483 (MH⁺).

N-({6,6-dimethyl-4-[7-(2-methyl-1H-benzimidazol-6-yl)-2,3-dihydro-1,4-benzoxazepin-4(5H)-yl]-5,6,7,8-tetrahydroquinazolin-2-yl}methyl)cyclobutanamine. Prepared as acetate salt according to the method of example 1 by using benzyl (4-chloro-6,6-dimethyl-5,6,7,8-tetrahydroquinazolin-2-yl)methyl(cyclobutyl)carbamate (reagent preparation 17) in step 5 followed by Cbz deprotection. ¹H NMR (400 MHz, methanol-d₄): 7.67 (s, 1H), 7.62 (s, 1H), 7.54-7.44 (m, 3H), 7.03 (d, 1H), 4.81 (s, 2H), 4.35 (m, 2H), 4.03 (m, 2H), 3.83 (s, 2H), 3.50 (m, 1H), 2.79 (t, 2H), 2.58 (s, 3H), 2.52 (s, 2H), 2.04 (m, 2H), 1.92 (s, 3H), 1.83 (m, 2H), 1.66 (m, 2H), 0.93 (s, 6H); MS (EI) for C₃₂H₃₈N₆O: 523 (MH⁺).

4-[7-(2-methyl-1H-benzimidazol-6-yl)-2,3-dihydro-1,4-benzoxazepin-4(5H)-yl]-N-phenylpyrimidin-2-amine. Prepared by the method of example 1 using 2-anilino-4-chloropyrimidine in step 5. ¹H NMR (400 MHz, DMSO-d6) δ 12.03 (br s, 1H), 9.01 (s, 1H), 7.98 (d, 1H), 7.50 (m, 9H), 7.01 (d, 1H), 6.89 (t, 1H), 4.87 (br s, 2H), 4.24 (s, 4H), 2.48 (s, 3H); MS (EI) for C₂₇H₂₄N₆O: 449.1 (MH⁺).

1-{6,6-dimethyl-4-[7-(2-methyl-1H-benzimidazol-6-yl)-2,3-dihydro-1,4-benzoxazepin-4(5H)-yl]-5,6,7,8-tetrahydroquinazolin-2-yl}-N,N-dimethylmethanamine. The trihydrochloride salt was prepared as in example 1 using 1-(4-chloro-6,6-dimethyl-5,6,7,8-tetrahydroquinazolin-2-yl)-N,N-dimethylmethanamine (reagent preparation 17) in step 5. ¹H NMR (400 MHz, CD₃OD) δ 8.03 (s, 1H), 7.97 (s, 1H), 7.91 (dd, 1H), 7.81 (d, 1H), 7.62 (dd, 1H), 7.07 (d, 1H), 5.32 (s, 2H), 4.62-4.55 (m, 4H), 4.39-4.31 (m, 2H), 2.88 (s, 6H), 2.97-2.80 (m, 2H), 2.62 (s, 2H), 1.71 (t, 2H), 0.92 (s, 6H); MS (ES) for C₃₀H₃₆N₆O: 497.2 (MH⁺).

4-(2-{[(3S)-3-fluoropyrrolidin-1-yl]methyl}-6,6-dimethyl-5,6,7,8-tetrahydroquinazolin-4-yl)-7-(2-methyl-1H-benzimidazol-6-yl)-2,3,4,5-tetrahydro-1,4-benzoxazepine. The trihydrochloride salt was prepared as in example 1 using (S)-4-chloro-2-((3-fluoropyrrolidin-1-yl)methyl)-6,6-dimethyl-5,6,7,8-tetrahydroquinazoline (reagent example 17) in step 5. ¹H NMR (400 MHz, CD₃OD) δ 7.99 (s, 1H), 7.92-7.86 (m, 1H), 7.84 (bs, 1H), 7.79 (d, 1H), 7.61 (dd, 1H), 7.09 (d, 1H), 5.41-5.20 (m, 1H), 5.19-5.02 (m, 2H), 4.64-4.55 (m, 2H), 4.55-4.48 (m, 2H), 4.24-4.13 (m, 2H), 3.88-3.50 (m, 4H), 2.88 (s, 3H), 2.90-2.79 (m, 2H), 2.64-2.51 (m, 2H), 2.25 (s, 2H), 1.70 (t, 2H), 0.91 (d, 6H); MS (ES) for C₃₂H₃₇FN₆O: 541.4 (MH⁺).

4-(2-{[(3R)-3-fluoropyrrolidin-1-yl]methyl}-6,6-dimethyl-5,6,7,8-tetrahydroquinazolin-4-yl)-7-(2-methyl-1H-benzimidazol-6-yl)-2,3,4,5-tetrahydro-1,4-benzoxazepine. The trihydrochloride salt was prepared as in example 1 using (R)-4-chloro-2-((3-fluoropyrrolidin-1-yl)methyl)-6,6-dimethyl-5,6,7,8-tetrahydroquinazoline (reagent preparation 17) in step 5. ¹H NMR (400 MHz, CD₃OD) δ 7.95 (s, 1H), 7.89-7.84 (m, 1H), 7.80 (s, 1H), 7.78 (s, 1H), 7.59 (dd, 1H), 7.09 (d, 1H), 5.44-5.23 (m, 1H), 5.00 (s, 2H), 4.52 (s, 2H), 4.50-4.45 (m, 2H), 4.16-4.07 (m, 2H), 3.59 (s, 4H), 2.88 (s, 3H), 2.91-2.78 (m, 2H), 2.55 (s, 2H), 2.36-2.19 (m, 2H), 1.70 (t, 2H), 0.90 (d, 6H); MS (ES) for C₃₂H₃₇FN₆O: 541.4 (MH⁺).

4-(6,6-dimethyl-2-pyridin-2-yl-5,6,7,8-tetrahydroquinazolin-4-yl)-7-(2-methyl-1H-benzimidazol-6-yl)-2,3,4,5-tetrahydro-1,4-benzoxazepine. The trihydrochloride salt was prepared as in example 1 using 4-chloro-6,6-dimethyl-2-(pyridin-2-yl)-5,6,7,8-tetrahydroquinazoline (reagent preparation 17) in step 5. ¹H NMR (400 MHz, d₆-DMSO) δ 8.80 (d, 1H), 8.48 (d, 1H), 8.10 (s, 1H), 8.01 (d, 1H), 7.98 (s, 1H), 7.85 (d, 1H), 7.79 (d, 1H), 7.69 (t, 1H), 7.57 (d, 1H), 6.99 (d, 1H), 5.26 (s, 2H), 4.55 (bs, 2H), 4.30 (bs, 2H), 2.94 (t, 2H), 2.85 (s, 3H), 2.64 (s, 2H), 1.62 (t, 2H), 0.92 (s, 6H); MS (ES) for C₃₂H₃₂N₆O: 517.3 (MH⁺).

2-{6,6-dimethyl-4-[7-(2-methyl-1H-benzimidazol-6-yl)-2,3-dihydro-1,4-benzoxazepin-4(5H)-yl]-5,6,7,8-tetrahydroquinazolin-2-yl}propan-2-ol. The dihydrochloride salt was prepared as in example 1 using 2-(4-chloro-6,6-dimethyl-5,6,7,8-tetrahydroquinazolin-2-yl)propan-2-ol (reagent preparation 17) in step 5. ¹H NMR (400 MHz, CD₃OD) δ 7.88 (s, 1H), 7.78 (s, 2H), 7.74 (d, 1H), 7.53 (dd, 1H), 7.01 (d, 1H), 5.16 (s, 2H), 4.54-4.46 (m, 2H), 4.31-4.24 (m, 2H), 2.89 (t, 2H), 2.85 (s, 3H), 2.61 (s, 2H), 1.70 (t, 2H), 1.39 (d, 6H), 0.96 (s, 6H); MS (ES) for C₃₀H₃₅N₅O₂: 498.2 (MH⁺).

N,N-dimethyl-1-{4-methyl-6-[7-(2-methyl-1H-benzimidazol-5-yl)-2,3-dihydro-1,4-benzoxazepin-4(5H)-yl]-5-(1-methylethyl)pyrimidin-2-yl}methanamine. Synthesized according to the method of example 1 using 1-(4-chloro-5-(4-fluorobenzyl)-6-methylpyrimidin-2-yl)-N,N-dimethylmethanamine (reagent preparation 17) in step 5. ¹H NMR (400 MHZ, CDCl₃): 9.84 (br, 1H), 7.69 (br, 1H), 7.41 (dd, 2H), 7.23 (br, 1H), 7.03-6.97 (m, 3H), 6.92-6.88 (m, 2H), 6.69 (br, 1H), 4.35 (s, 2H), 4.21 (tr, 2H), 3.91 (s, 2H), 3.83 (tr, 2H), 3.60 (s, 2H), 2.67 (s, 3H), 2.39 (s, 6H), 2.23 (s, 3H). MS (EI) for C₃₂H₃₃N₆OF: 537 (MH⁺).

N,N-dimethyl-1-{4-methyl-6-[7-(2-methyl-1H-benzimidazol-5-yl)-2,3-dihydro-1,4-benzoxazepin-4(5H)-yl]-5-(1-methylethyl)pyrimidin-2-yl}methanamine. Synthesized according to the method of example 1 using 1-(4-chloro-5-isopropyl-6-methylpyrimidin-2-yl)-N,N-dimethylmethanamine (reagent preparation 17) in step 5. ¹H NMR (400 MHZ, DMSO-d₆): 10.85 (br, 1H), 8.02 (s, 1H), 7.99 (s, 1H), 7.83 (m, 2H), 7.55 (d, 1H), 6.98 (d, 1H), 5.04 (br s, 2H), 4.47 (br s, 2H), 4.43 (s, 2H), 3.95 (br s, 2H), 3.14 (m, 1H), 2.81 (s, 3H), 2.67 (s, 6H), 2.56 (s, 3H), 1.32 (d, 6H). MS (EI) for C₂₈H₃₄N₆O: 471 (MH⁺).

N-({5-[(4-fluorophenyl)methyl]-4-methyl-6-[7-(2-methyl-1H-benzimidazol-6-yl)-2,3-dihydro-1,4-benzoxazepin-4(5H)-yl]pyrimidin-2-yl}methyl)cyclopropanamine. Prepared as acetate salt according to the method of example 1 by using N-((4-chloro-5-(4-fluorobenzyl)-6-methylpyrimidin-2-yl)methyl)cyclopropanamine (reagent preparation 17) in step 5 ¹H NMR (400 MHz, methanol-d₄): 7.59 (s, 1H), 7.53 (d, 1H), 7.44 (dd, 1H), 7.34 (d, 1H), 7.10 (m, 3H), 6.99 (m, 3H), 4.67 (s, 2H), 4.25 (m, 2H), 4.05 (s, 2H), 3.90 (m, 2H), 3.85 (s, 2H), 2.66 (s, 3H), 2.22 (s, 3H), 2.17 (m, 1H), 1.94 (s, 3H), 0.37 (m, 2H), 0.32 (m, 2H); MS (EI) for C₃₃H₃₃FN₆O: 549 (MH⁺).

4-[7-(2-methyl-1H-benzimidazol-6-yl)-2,3-dihydro-1,4-benzoxazepin-4(5H)-yl]pyridin-2-amine. Prepared according to the method of example 1 using 4-chloro-2-nitropyridine in step 5 followed by nitro group reduction using palladium on carbon hydrogenation in methanol at 35 psi. ¹H NMR (400 MHz, CD₃OD) δ 7.76-7.62 (m, 2H), 7.60-7.37 (m, 4H), 7.07 (d, 1H), 6.57 (dd, 1H), 6.06 (d, 1H), 4.82 (s, 2H), 4.29-4.18 (m, 2H), 4.06-3.97 (m, 2H), 2.59 (s, 3H).

### Example 2

### Methyl (6-{4-[6,7-bis(methyloxy)quinolin-4-yl]-2,3,4,5-tetrahydro-1,4-benzoxazepin-7-yl}-1H-benzimidazol-2-yl)carbamate

STEP 1: 1,1-dimethylethyl 7-bromo-2,3-dihydro-1,4-benzoxazepine-4(5H)-carboxylate (10 g, 30.5 mmol) was taken into hot ethanol (10 mL) followed by addition of 4 M hydrogen chloride in dioxane solution (2.1 eq, 16 mL) and the resulting solution was allowed to slowly cool to ambient temperature over one hour. An excess of ethyl ether was then added and the resulting slurry was filtered. The filter cake was washed with ethyl ether and dried to give 7-bromo-2,3,4,5-tetrahydro-1,4-benzoxazepine hydrochloride (7.9 g, 98% yield) as a colorless crystalline solid. MS (EI) for C₉H₁₀NOBr: 229 (MH⁺).

STEP 2: A mixture of 7-bromo-2,3,4,5-tetrahydro-1,4-benzoxazepine hydrochloride (300 mg, 1.13 mmol), 4-chloro-6,7-dimethoxyquinoline (253 mg, 1.13 mmol), and potassium carbonate (470 mg, 3.40 mmol) in N-methylpyrrolidine (2 mL) was stirred at 160° C. for 17 h. Ethyl acetate (75 mL) was added and the mixture was washed with water (3×25 mL) and brine (25 mL), dried over sodium sulfate, and concentrated. Column chromatography on silica (dichloromethane:methanol 95:5) afforded 4-[6,7-bis(methyloxy)quinolin-4-yl]-7-bromo-2,3,4,5-tetrahydro-1,4-benzoxazepine (80 mg). MS (EI) for C₂₀H₁₉BrN₂O₃: 416 (MH⁺).

STEP 3: A mixture 4-[6,7-bis(methyloxy)quinolin-4-yl]-7-bromo-2,3,4,5-tetrahydro-1,4-benzoxazepine (78 mg, 0.19 mmol), 4-amino-3-nitrophenylboronic acid pinacol ester (59 mg, 0.23 mmol), potassium carbonate (105 mg, 0.76 mmol), and dichloro[1,1′-bis(diphenylphosphino)ferrocene]palladium (II) dichloromethane adduct (20 mg, 0.02 mmol) in dimethoxyethane (3 mL) was stirred at 80° C. for 3 h. Ethyl acetate (50 mL) was added and the mixture was washed with water (20 mL), and brine (20 mL), dried over sodium sulfate, and concentrated. Column chromatography on silica (dichloromethane: methanol 95:5) gave 4-{4-[6,7-bis(methyloxy)quinolin-4-yl]-2,3,4,5-tetrahydro-1,4-benzoxazepin-7-yl}-2-nitroaniline (56 mg, 63% yield). MS (EI) for C₂₆H₂₄N₄O₅: 473 (MH⁺).

STEP 4: A solution of 4-{4-[6,7-bis(methyloxy)quinolin-4-yl]-2,3,4,5-tetrahydro-1,4-benzoxazepin-7-yl}-2-nitroaniline (56 mg, 0.12 mmol) in methanol (20 mL) was hydrogenated at 30 psi over 10% Pd—C (25 mg) for 4 h. The catalyst was filtered off, and the filtrate was concentrated to give 4-{4-[6,7-bis(methyloxy)quinolin-4-yl]-2,3,4,5-tetrahydro-1,4-benzoxazepin-7-yl}benzene-1,2-diamine (40 mg, 77% yield) as a brown oil. MS (EI) for C₂₆H₂₆N₄O₃: 443 (MH⁺).

STEP 5: To a solution of 4-{4-[6,7-bis(methyloxy)quinolin-4-yl]-2,3,4,5-tetrahydro-1,4-benzoxazepin-7-yl}benzene-1,2-diamine (40 mg, 0.09 mmol) in acetic acid (2 mL) was added 1,3-bis(methoxycarbonyl)-2-methyl-2-thiopseudourea (19 mg, 0.09 mmol) and the reaction mixture was stirred at 80° C. for 30 min. After cooling to room temperature the mixture was concentrated, and purified by preparative reverse phase HPLC to provide methyl (6-{4-[6,7-bis(methyloxy)quinolin-4-yl]-2,3,4,5-tetrahydro-1,4-benzoxazepin-7-yl}-1H-benzimidazol-2-yl)carbamate (17 mg, 25% yield) as a brown solid. ¹H NMR (400 MHz, CD₃OD); 8.30 (d, 1H), 7.78 (m, 2H), 7.68 (m, 2H), 7.60 (m, 1H), 7.27 (d, 2H), 7.11 (d, 1H), 7.04 (d, 1H), 5.17 (s, 2H), 4.64 (m, 2H), 4.25 (m, 2H), 4.01 (s, 3H), 3.96 (s 3H), 3.63 (s, 3H); MS (EI) for C₂₉H₂₇N₅O₅: 526 (MH⁺).

Using analogous synthetic techniques and substituting with alternative starting reagents in steps 2, 3 or 5 the following compounds of the invention were prepared. Alternative starting materials were obtained commercially unless otherwise indicated.

Methyl[6-(4-quinolin-4-yl-2,3,4,5-tetrahydro-1,4-benzoxazepin-7-yl)-1H-benzimidazol-2-yl]carbamate. Synthesized according to the method of example 2 using 4-chloroquinoline in step 2. ¹H NMR (400 MHz, d₆-DMSO): 8.72 (d, 1H), 8.03 (d, 1H), 7.96 (d, 1H), 7.69 (m, 3H), 7.53 (m, 2H), 7.46 (d, 1H), 7.39 (d, 1H), 7.11 (d, 1H) 7.02 (d, 11-1) 4.63 (s, 2H), 4.38 (br s, 2H), 3.81 (br s, 2H), 3.77 (s, 3H); MS (EI) for C₂₇H₂₃N₅O₃: 466 (MH⁺).

Methyl[1-methyl-5-(4-quinolin-4-yl-2,3,4,5-tetrahydro-1,4-benzoxazepin-7-yl)-1H-benzimidazol-2-yl]carbamate. Synthesized according to the method of example 2 using 4-chloroquinoline in step 2 and 4-methylamino-3-nitrophenylboronic acid pinacol ester (Bioorg. Med. Chem. Lett. 2007, 17(19), 5406-5409) in step 3. ¹H NMR (400 MHz, d₆-DMSO): 8.62 (d, 1H), 8.01 (d, 1H), 7.94 (d, 1H), 7.70 (m, 3H), 7.64 (m, 2H), 7.51 (m, 4H), 7.13 (d, 1H) 7.01 (d, 1H) 4.64 (s, 2H), 4.39 (br s, 2H), 3.82 (br s, 2H), 3.64 (s, 3H), 3.54 (s, 3H); MS (EI) for C₂₈H₂₅N₅O₃: 480 (MH⁺).

1-Methyl-5-(4-quinolin-4-yl-2,3,4,5-tetrahydro-1,4-benzoxazepin-7-yl)-1H-benzimidazol-2-amine. Synthesized according to the method of example 2 using 4-chloroquinoline in step 2 and 4-methylamino-3-nitrophenylboronic acid pinacol ester (Bioorg. Med. Chem. Lett. 2007, 17(19), 5406-5409) in step 3. Material obtained as a co-product in the formation of methyl[1-methyl-5-(4-quinolin-4-yl-2,3,4,5-tetrahydro-1,4-benzoxazepin-7-yl)-1H-benzimidazol-2-yl]carbamate. ¹H NMR (400 MHz, d₆-DMSO): 8.62 (d, 1H), 8.04 (d, 1H), 7.96 (d, 1H), 7.68 (m, 2H), 7.52 (m, 2H), 7.42 (s, 1H), 7.13 (dt, 2H) 7.08 (d, 1H), 7.04 (d, 1H), 6.48 (br s, 2H), 4.68 (s, 2H), 4.38 (br s, 2H), 3.81 (br s, 2H), 3.52 (s, 3H); MS (EI) for C₂₆H₂₃N₅O: 422 (MH⁺).

Methyl[1-methyl-6-(4-quinolin-4-yl-2,3,4,5-tetrahydro-1,4-benzoxazepin-7-yl)-1H-benzimidazol-2-yl]carbamate. Synthesized according to the method of example 2 using 4-chloroquinoline in step 2 and 4-nitro-3-methylaminophenylboronic acid pinacol ester (Bioorg. Med. Chem. Lett. 2007, 17(19), 5406-5409) in step 3. ¹H NMR (400 MHz, d₆-DMSO): 8.63 (d, 1H), 8.02 (d, 1H), 7.94 (d, 1H), 7.71 (m, 2H), 7.62 (s, 1H), 7.50 (m, 4H), 7.13 (d, 1H) 7.03 (d, 1H) 4.64 (s, 2H), 4.40 (br s, 2H), 3.81 (br s, 2H), 3.63 (s, 3H), 3.55 (s, 3H); MS (EI) for C₂₈H₂₅N₅O₃: 480 (MH⁺).

2-(Methyloxy)ethyl[6-(4-quinolin-4-yl-2,3,4,5-tetrahydro-1,4-benzoxazepin-7-yl)-1H-benzimidazol-2-yl]carbamate. Synthesized according to the method of example 2 using 4-chloroquinoline in step 2 and 1,3-bis-[2-(methoxy)-ethoxycarbonyl]-2-methyl-2-thiopseudourea (reagent preparation 10) in step 5. ¹H NMR (400 MHz, d₆-DMSO): 8.62 (d, 1H), 8.03 (d, 1H), 7.94 (d, 1H), 7.71 (m, 3H), 7.53 (m, 2H), 7.47 (d, 1H), 7.38 (m, 1H), 7.11 (d, 1H) 7.01 (d, 1H) 4.64 (s, 2H), 4.31 (br s, 2H), 3.83 (br s, 2H), 3.61 (m, 2H), 3.34 (br s, 2H), 3.30 (s, 3H); MS (EI) for C₂₉H₂₇N₅O₄: 510 (MH⁺).

4-Piperidin-1-yl-N-[6-(4-quinolin-4-yl-2,3,4,5-tetrahydro-1,4-benzoxazepin-7-yl)-1H-benzimidazol-2-yl]butanamide. Synthesized according to the method of example 2 using 4-chloroquinoline in step 2 and 1,3-bis-[3-(piperidin-1-yl)propylcarbonyl]-2-methyl-2-thiopseudourea (reagent preparation 10) in step 5. ¹H NMR (400 MHz, d₆-DMSO): 9.22 (br s, 1H), 8.67 (d, 1H), 8.33 (d, 1H), 7.95 (m, 3H), 7.78 (s, 1H), 7.73 (t, 1H), 7.57 (m, 3H), 6.98 (d, 2H), 5.31 (s, 2H), 4.61 (br s, 2H), 4.41 (br s, 2H), 3.43 (d, 2H), 3.11 (m, 2H), 2.91 (m, 2H), 2.59 (m, 2H), 2.02 (m, 2H), 1.81 (m, 2H); MS (EI) for C₃₄H₃₆N₆O₂: 561 (MH⁺).

Methyl[6-(4-{5-[(4-fluorophenyl)methyl]-6-methylpyrimidin-4-yl}-2,3,4,5-tetrahydro-1,4-benzoxazepin-7-yl)-1H-benzimidazol-2-yl]carbamate. Synthesized according to the method of example 2 using 4-chloro-5-(4-fluorobenzyl)-6-methylpyrimidine in step 2. ¹H NMR (400 MHz, DMSO-D₆): 11.70 (bs, 1H), 8.50 (s, 1H), 7.49 (s, 1H), 7.38-7.46 (m, 2H), 7.09-7.16 (m, 5H), 6.99 (d, 1H), 6.88 (s, 1H), 4.48 (s, 2H), 4.23-4.30 (m, 2H), 4.00 (s, 2H), 3.74-3.81 (m, 5H), 2.16 (s; 3H); MS (EI) for C₃₀H₂₇FN₆O₃: 539 (MH⁺).

Methyl[6-(4-pyrimidin-4-yl-2,3,4,5-tetrahydro-1,4-benzoxazepin-7-yl)-1H-benzimidazol-2-yl]carbamate. Prepared according to the method of example 2 by using 4-chloropyrimidine in step 2. ¹H NMR (400 MHz, DMSO-d₆): 8.49 (s, 1H), 8.17 (d, 1H), 7.80 (s, 1H), 7.60 (s, 1H), 7.44 (m, 2H), 7.34 (m, 1H), 7.03 (m, 2H), 4.87 (s, 2H), 4.15 (s, 4H), 3.76 (s, 3H); MS (EI) for C₂₂H₂₀N₆O₃: 417 (MH⁺).

Methyl{6-[4-(7H-pyrrolo[2,3-d]pyrimidin-4-yl)-2,3,4,5-tetrahydro-1,4-benzoxazepin-7-yl]-1H-benzimidazol-2-yl}carbamate. Prepared according to the method of example 2 by using 4-chloropyrrolo[2,3-d]pyrimidine in step 2. ¹H NMR (400 MHz, DMSO-d₆): 12.25 (br. s, 1H), 8.30 (s, 1H), 7.78 (d, 1H), 7.67 (s, 1H), 7.57 (d, 1H), 7.47 (m, 2H), 7.35 (m, 1H), 7.04 (d, 1H), 6.92 (s, 1H), 5.25 (s, 2H), 4.39 (m, 4H), 3.84 (s, 3H); MS (EI) for C₂₄H₂₁N₇O₃: 456 (MH⁺).

Methyl{6-[4-(3-methylpyridin-2-yl)-2,3,4,5-tetrahydro-1,4-benzoxazepin-7-yl]-1H-benzimidazol-2-yl}carbamate. Prepared according to the method of example 2 by using 2-chloro-3-methylpyridine in step 2. ¹H NMR (400 MHz, DMSO-d₆): 11.69 (br. s, 1H), 8.07 (m, 1H), 7.59 (d, 1H), 7.54 (d, 1H), 7.51 (d, 1H), 7.44 (m, 2H), 7.32 (dd, 1H), 7.06 (d, 1H), 6.89 (m, 1H), 4.42 (s, 2H), 4.28 (m, 2H), 3.76 (s, 3H), 3.65 (m, 2H), 2.30 (s, 3H); MS (EI) for C₂₄H₂₃N₅O₃: 430 (MH⁺).

Methyl{6-[4-(2-methylquinazolin-4-yl)-2,3,4,5-tetrahydro-1,4-benzoxazepin-7-yl]-1H-benzimidazol-2-yl}carbamate. Prepared according to the method of example 2 by using 4-chloro-2-methylquinazoline in step 2. ¹H NMR (400 MHz, DMSO-d₆): 7.99 (d, 1H), 7.74 (m, 1H), 7.68 (d, 1H), 7.61 (m, 2H), 7.44 (m, 3H), 7.34 (d, 1H), 7.00 (d, 1H), 5.01 (s, 2H), 4.42 (m, 2H), 4.17 (m, 2H), 3.74 (s, 3H), 2.45 (s, 3H); MS (EI) for C₂₇H₂₄N₆O₃: 481 (MH⁺).

Methyl[6-(4-quinazolin-4-yl-2,3,4,5-tetrahydro-1,4-benzoxazepin-7-yl)-1H-benzimidazol-2-yl]carbamate. Prepared as trifluoroacetate salt according to the method of example 2 by using 4-chloroquinazoline in step 2. ¹H NMR (400 MHz, DMSO-d₆): 8.84 (s, 1H), 8.31 (s, 1H), 7.83 (d, 1H), 7.78-7.69 (m, 3H), 7.56-7.46 (m, 3H), 7.01 (d, 1H), 5.45 (s, 2H), 4.63 (m, 2H), 4.54 (m, 2H), 3.81 (s, 3H); MS (EI) for C₂₆H₂₂N₆O₃: 4678 (MH⁺).

Methyl[6-(4-isoquinolin-1-yl-2,3,4,5-tetrahydro-1,4-benzoxazepin-7-yl)-1H-benzimidazol-2-yl]carbamate. Prepared as trifluoroacetate salt according to the method of example 2 by using 1-chloroisoquinoline in step 2. ¹H NMR (400 MHz, methanol-d₄): 8.26 (d, 1H), 8.04 (d, 1H), 7.96 (m, 1H), 7.83 (m, 2H), 7.68 (m, 5H), 7.56 (d, 1H), 7.21 (d, 1H), 5.14 (s, 2H), 4.67 (m, 2H), 4.14 (m, 2H), 3.96 (s, 3H); MS (EI) for C₂₇H₂₃N₅O₃: 466 (MH⁺).

Methyl (6-{4-[6,7-bis(methyloxy)quinazolin-4-yl]-2,3,4,5-tetrahydro-1,4-benzoxazepin-7-yl}-1H-benzimidazol-2-yl)carbamate. Prepared according to the method of example 2 by using 4-chloro-6,7-dimethoxyquinazoline in step 2. ¹H NMR (400 DMSO-D₆): 8.49 (1H), 7.73 (br, 1H), 7.62 (br, 1H), 7.49 (dd, 1H), 7.44 (d, 1H), 7.35 (dd, 1H), 7.20 (s, 1H), 7.09 (d, 2H), 5.02 (s, 2H), 4.53 (m, 2H), 4.06 (m, 2H), 3.90 (s, 3H), 3.75 (s, 3H), 3.54 (s, 3H), MS (EI) for C₂₈H₂₆N₆O₅: 527 (MH⁺).

Methyl (6-{4-[6-(methyloxy)quinazolin-4-yl]-2,3,4,5-tetrahydro-1,4-benzoxazepin-7-yl}-1H-benzimidazol-2-yl)carbamate. Prepared according to the method of example 2 by using 4-chloro-6-methoxyquinazoline in step 2. ¹H NMR (400 DMSO-D₆): 8.53 (1H), 7.75 (d, 2H), 7.62 (br, 1H), 7.52 to 7.42 (m, 3H), 7.35 (dd, 1H), 7.17 (s, 1H), 7.05 (d, 1H), 5.04 (s, 2H), 4.53 (m, 2H), 4.11 (m, 2H), 3.78 (s, 3H), 3.58 (s, 3H), MS (EI) for C₂₇H₂₄N₆O₄: 497 (MH⁺).

### Example 3

### 5-(4-{5-[(4-Fluorophenyl)methyl]-6-methylpyrimidin-4-yl}-2,3,4,5-tetrahydro-1,4-benzoxazepin-7-yl)-1,3-thiazol-2-amine

STEP 1: A mixture of N-(5-bromo-1,3-thiazol-2-yl)acetamide (1.00 g, 4.5 mmol), (4-{[(1,1-dimethylethyl)oxy]carbonyl}-2,3,4,5-tetrahydro-1,4-benzoxazepin-7-yl)boronic acid (example 1, step 2) (1.59 g, 5.4 mmol), potassium carbonate (2.50 g, 18 mmol), [1,1′-bis(diphenylphosphino)ferrocene]dichloropalladium(II) (0.33 g, 0.45 mmol), 1,4-dioxane (20 mL), and water (2 mL) was degassed with nitrogen for 2 min, and then stirred at 95° C. for 16 h. The reaction mixture was cooled, diluted with ethyl acetate (100 mL), and filtered through celite. The filtrate was washed with brine (2×30 mL), dried over sodium sulfate, and concentrated. Column chromatography on silica (gradient 20-85% ethyl acetate in hexane) gave 1,1-dimethylethyl 7-[2-(acetylamino)-1,3-thiazol-5-yl]-2,3-dihydro-1,4-benzoxazepine-4(5H)-carboxylate (0.99 g, 56% yield). MS (EI) for C₁₉H₂₃N₃O₄: 390 (MH⁺).

STEP 2: A solution of 1,1-dimethylethyl 7-[2-(acetylamino)-1,3-thiazole-5-yl]-2,3-dihydro-1,4-benzoxazepine-4(5H)-carboxylate (300 mg, 0.77 mmol) in a mixture of methanol (2 mL) and 4 N hydrogen chloride in dioxane (2 mL) was refluxed for 1 min. After cooling to room temperature the reaction mixture was concentrated, and azeotroped with methanol (3×) to give N-[5-(2,3,4,5-tetrahydro-1,4-benzoxazepin-7-yl)-1,3-thiazol-2-yl]acetamide hydrochloride (221 mg, 88% yield) as a colorless solid. MS (EI) for C₁₄H₁₅N₃O₂S: 290 (MH⁺).

STEP 3: A mixture of N-[5-(2,3,4,5-tetrahydro-1,4-benzoxazepin-7-yl)-1,3-thiazol-2-yl]acetamide hydrochloride (220 mg, 0.68 mmol), 4-chloro-5-[(4-fluorophenyl)-methyl]-6-methylpyrimidine (reagent preparation 5) (152 mg, 0.64 mmol) and diisopropylethylamine (500 mg, 3.87 mmol) in N-methylpyrrolidine (4 mL) was heated in a microwave reactor at 120° C. for 3 h. The reaction mixture was concentrated, diluted with water (10 mL), the precipitate was filtered off, washed with water (2×5 mL) and methanol (5 mL), and dried to provide N-[5-(4-{5-[(4-fluorophenyl)methyl]-6-methylpyrimidine-4-yl}-2,3,4,5-tetrahydro-1,4-benzoxazepin-7-yl)-1,3-thiazol-2-yl]acetamide (147 mg, 47% yield) as a off-white solid. MS (EI) C₂₆H₂₄FN₅O₂S: 490 (MH⁺).

STEP 4: A solution of N-[5-(4-{5-[(4-fluorophenyl)methyl]-6-methylpyrimidine-4-yl}-2,3,4,5-tetrahydro-1,4-benzoxazepin-7-yl)-1,3-thiazol-2-yl]acetamide (75 mg, 0.15 mmol) in 6 N hydrochloric acid (4 mL) was stirred at 95° C. for 15 h. After cooling to room temperature the mixture was neutralized with 50% aqueous sodium hydroxide, concentrated to dryness, and the solid residue extracted with ethanol (3×10 mL). Evaporation of the solvent and purification of the residue by preparative HPLC afforded 5-(4-{5-[(4-fluorophenyl)methyl]-6-methylpyrimidin-4-yl}-2,3,4,5-tetrahydro-1,4-benzoxazepin-7-yl)-1,3-thiazol-2-amine (43 mg, 62% yield) as a colorless solid. ¹H NMR (400 MHz, CD₃OD): 8.46 (s, 1H), 7.21 (m, 1H), 7.11 (m, 4H), 6.98 (s, 1H), 6.90 (d, 1H), 6.61 (m, 1H), 4.46 (s, 2H), 4.26 (ms, 2H), 3.97 (s, 2H), 3.87 (m, 2H), 2.21 (s, 3H); MS (EI) C₂₄H₂₂FN₅OS: 448 (MH⁺).

Using analogous synthetic techniques and substituting with alternative starting reagents in steps 1 or 3 and conducting protecting group removal step 4 as required according to literature techniques appropriate for a given protecting group (see for example: Greene and Wuts, Protective Groups in Organic Synthetic, Wiley-Interscience) the following compounds of the invention were prepared. Alternative starting materials were obtained commercially unless otherwise indicated.

4-{5-[(4-Fluorophenyl)methyl]-6-methylpyrimidin-4-yl}-7-(2-methyl-3H-imidazo[4,5-b]pyridin-6-yl)-2,3,4,5-tetrahydro-1,4-benzoxazepine. Synthesized according to the method of example 3 using isobutyl 6-bromo-2-methyl-3H-imidazo[4,5-b]pyridine-3-carboxylate (reagent preparation 11) in step 1. ¹H NMR (400 MHz, CDCl₃): 8.62 (s, 1H), 7.42 (d, 2H), 7.11 (d, 1H), 7.03 (m, 2H), 6.95 (t, 2H), 6.79 (s, 1H), 4.47 (s, 2H), 4.26 (br s, 2H), 3.97 (s, 2H), 3.88 (br s, 2H), 2.77 (s, 3H), 2.27 (s, 3H); MS (EI) for C₂₈H₂₅FN₆O: 481 (MH⁺).

7-(1H-Benzimidazol-6-yl)-4-quinazolin-4-yl-2,3,4,5-tetrahydro-1,4-benzoxazepine. Prepared according to the method of example 3 by using 6-bromo-1H-benzo[d]imidazole in step 1 and 4-chloroquinazoline in step 3. ¹H NMR (400 MHz, methanol-d₄): 8.53 (s, 1H), 8.21 (s, 1H), 8.12 (d, 1H), 7.80 (m, 3H), 7.68 (d, 1H), 7.63 (m, 1H), 7.53 (m, 3H), 7.07 (d, 1H), 5.13 (s, 2H), 4.49 (m, 2H), 4.30 (m, 2H), 1.96 (s, 3H); MS (EI) for C₂₄H₁₉N₅O: 394 (MH⁺).

4-{5-[(4-Fluorophenyl)methyl]-6-methylpyrimidin-4-yl}-7-[4-(1H-imidazol-2-yl)phenyl]-2,3,4,5-tetrahydro-1,4-benzoxazepine. Prepared according to the method of example 3 by using isobutyl 2-(4-bromophenyl)-1H-imidazole-1-carboxylate in step 1 and 4-chloro-5-(4-fluorobenzyl)-6-methylpyrimidine (reagent preparation 5) in step 3. ¹H NMR (400 DMSO-D₆): 8.51 (1H), 8.01 (d, 2H), 7.54 (d, 3H), 7.20 (br 1H), 7.12 (d, 5H), 7.02 (d, 1H), 6.91 (br, 1H), 4.49 (s, 2H), 4.29 (m, 2H), 3.97 (s, 2H), 3.78 (s, 2H), 2.16 (s, 3H), MS (EI) for C₃₀H₂₆N₅FO: 492 (MH⁺).

7-(1-Ethyl-1H-benzimidazol-5-yl)-4-quinolin-4-yl-2,3,4,5-tetrahydro-1,4-benzoxazepine. Prepared as the trifluoroacetate salt according to the method of example 3 by using 5-bromo-1-ethyl-1H-benzimidazole (reagent preparation 12) in step 1 and 4-chloroquinoline in step 3. ¹H NMR (400 MHz, d₆-DMSO): 8.57 (d, 1H), 8.31 (d, 1H), 8.07-7.91 (m, 6H), 7.82 (s, 1H), 7.72-7.62 (m, 2H), 7.03-6.98 (m, 2H), 5.31 (s, 2H), 4.63 (t, 1H), 4.47-4.38 (m, 4H), 1.51 (t, 3H); MS (EI) for C₂₇H₂₄N₄O: 421 (MH⁺).

7-(2-Methyl-1,3-benzothiazol-5-yl)-4-quinolin-4-yl-2,3,4,5-tetrahydro-1,4-benzoxazepine. Prepared as the trifluoroacetate salt according to the method of example 3 by using 5-bromo-2-methylbenzothiazole in step 1 and 4-chloroquinoline in step 3. ¹H NMR (400 MHz, d₆-DMSO): 8.57 (d, 1H), 8.33 (d, 1H), 8.27 (s, 1H), 8.14 (d, 1H), 8.06 (s, 1H), 8.01-7.92 (m, 2H), 7.77 (d, 1H), 7.71-7.65 (m, 2H), 7.03-6.98 (m, 2H), 5.31 (s, 2H), 4.62 (t, 2H), 4.42 (t, 2H), 2.84 (s, 3H); MS (EI) for C₂₆H₂₁N₃OS: 424 (MH⁺).

7-(1,3-Benzothiazol-5-yl)-4-quinolin-4-yl-2,3,4,5-tetrahydro-1,4-benzoxazepine. Prepared as the trifluoroacetate salt according to the method of example 3 by using 5-bromobenzothiazole in step 1 and 4-chloroquinoline in step 3. ¹H NMR (400 MHz, d₆-DMSO): 9.47 (s, 1H), 8.57 (d, 1H), 8.46 (s, 1H), 8.33 (d, 1H), 8.29 (d, 1H), 8.11 (s, 1H), 8.01-7.92 (m, 2H), 7.88 (d, 1H), 7.73-7.66 (m, 2H), 7.03-6.99 (m, 2H), 5.31 (s, 2H), 4.63 (t, 2H), 4.42 (t, 2H); MS (EI) for C₂₅H₁₉N₃OS: 410 (MH⁺).

7-(1-Methyl-1H-benzimidazol-5-yl)-4-quinolin-4-yl-2,3,4,5-tetrahydro-1,4-benzoxazepine. Prepared as the acetate salt according to the method of example 3 by using 5-bromo-1-methylbenzimidazole in step 1 and 4-chloroquinoline in step 3. ¹H NMR (400 MHz, d₆-DMSO): 8.61 (d, 1H), 8.23 (s, 1H), 8.09 (d, 1H), 7.97-7.93 (m, 2H), 7.81 (s, 1H), 7.75 (t, 1H), 7.68-7.59 (m, 3H), 7.56-7.50 (m, 1H), 7.07 (d, 1H), 7.02 (d, 1H), 4.81 (s, 2H), 4.43 (t, 2H), 3.95 (t, 2H), 3.88 (s, 3H); MS (EI) for C₂₆H₂₂N₄O: 407 (MH⁺).

7-(1H-Benzimidazol-6-yl)-4-quinolin-4-yl-2,3,4,5-tetrahydro-1,4-benzoxazepine. Prepared as the trifluoroacetate salt according to the method of example 3 by using 5-bromobenzimidazole in step 1 and 4-chloroquinoline in step 3. ¹H NMR (400 MHz, d₆-DMSO): 9.02 (br. s, 1H), 8.57 (d, 1H), 8.33 (d, 1H), 8.03-7.92 (m, 4H), 7.87-7.76 (m, 2H), 7.71-7.61 (m, 2H), 7.04-6.97 (m, 2H), 5.31 (s, 2H), 4.63 (t, 2H), 4.41 (t, 2H); MS (EI) for C₂₅H₂₀N₄O: 393 (MH⁺).

4-Quinolin-4-yl-7-(3-thienyl)-2,3,4,5-tetrahydro-1,4-benzoxazepine. Prepared as the trifluoroacetate salt according to the method of example 3 by using thiophen-3-ylboronic acid in step 1 and 4-chloroquinoline in step 3. ¹H NMR (400 MHz, d₆-DMSO): 8.56 (d, 1H), 8.32 (d, 1H), 8.01-7.91 (m, 3H), 7.85 (s, 1H), 7.71-7.65 (m, 2H), 7.63-7.58 (m, 2H), 6.97-6.91 (m, 2H), 5.25 (s, 2H), 4.60 (t, 2H), 4.40 (t, 2H); MS (EI) for C₂₂H₁₈N₂OS: 359 (MH⁺).

7-Quinolin-3-yl-4-quinolin-4-yl-2,3,4,5-tetrahydro-1,4-benzoxazepine. Prepared as the trifluoroacetate salt according to the method of example 3 by using quinolin-3-ylboronic acid in step 1 and 4-chloroquinoline in step 3. ¹H NMR (400 MHz, d₆-DMSO): 9.39 (s, 1H), 8.77 (s, 1H), 8.56 (d, 1H), 8.34 (d, 1H), 8.21 (s, 1H), 8.11 (d, 2H), 8.02-7.94 (m, 2H), 7.85-7.80 (m, 2H), 7.74-7.68 (m, 2H), 7.09 (d, 1H), 7.02 (d, 1H), 5.34 (s, 2H), 4.67 (t, 2H), 4.44 (t, 2H); MS (EI) for C₂₇H₂₁H₃O: 404 (MH⁺).

7-(1-Benzothien-2-yl)-4-quinolin-4-yl-2,3,4,5-tetrahydro-1,4-benzoxazepine. Prepared as the trifluoroacetate salt according to the method of example 3 by using benzothiophen-2-ylboronic acid in step 1 and 4-chloroquinoline in step 3. ¹H NMR (400 MHz, d₆-DMSO): 8.58 (d, 1H), 8.31 (d, 1H), 8.06 (s, 1H), 8.01-7.92 (m, 3H), 7.88-7.84 (m, 2H), 7.72-7.65 (m, 2H), 7.44-7.34 (m, 2H), 7.00 (d, 1H), 6.96 (d, 1H), 5.29 (s, 2H), 4.63 (t, 2H), 4.39 (t, 2H); MS (EI) for C₂₆H₂₀N₂OS: 409 (MH⁺).

N-[2-chloro-5-(4-{2-[(dimethylamino)methyl]-6,6-dimethyl-5,6,7,8-tetrahydroquinazolin-4-yl}-2,3,4,5-tetrahydro-1,4-benzoxazepin-7-yl)pyridin-3-yl]methanesulfonamide. Prepared according to the method of example 3 by using N-(5-bromo-2-chloropyridin-3-yl)methanesulfonamide (reagent preparation 24) in step 1 and 1-(4-chloro-6,6-dimethyl-5,6,7,8-tetrahydroquinazolin-2-yl)-N,N-dimethylmethanamine (reagent preparation 17) in step 3. ¹H NMR (400 MHz, DMSO-d6) δ 8.12 (s, 1H), 7.88 (d, 1H), 7.64 (d, 1H), 7.45 (dd, 1H), 7.03 (d, 1H), 4.70 (s, 2H), 4.37-4.30 (m, 2H), 3.93-3.86 (m, 2H), 3.69 (s, 2H), 2.89 (s, 3H), 2.70 (t, 2H), 2.43 (s, 2H), 2.37 (s, 6H), 1.59 (t, 2H), 0.91-0.82 (m, 6H); MS (EI) for C₂₈H₃₅ClN₆O₃S: 571, 573 (Cl isotopes, MH⁺).

7-(1-methyl-1H-pyrrolo[2,3-b]pyridin-5-yl)-4-quinolin-4-yl-2,3,4,5-tetrahydro-1,4-benzoxazepine. Synthesized according to the method of example 3 using 5-bromo-1-methyl-1H-indole (reagent preparation 21) in step 1 and 4-chloroquinoline in step 3. ¹H NMR (400 MHz, DMSO-d₆): 8.62 (d, 1H), 8.58 (d, 1H), 8.23 (d, 1H), 8.01 (d, 1H), 7.94 (d, 1H), 7.74 (d, 1H), 7.68 (t, 1H), 7.62 (dd, 1H), 7.58 (dd, 1H), 7.53 (t, 1H), 7.12 (d, 1H), 7.03 (d, 1H), 6.52 (d, 1H), 4.66 (s, 2H), 4.38 (m, 2H), 3.85 (s, 3H), 3.82 (m, 2H); MS (EI) for C₂₆H₂₂N₄O₂: 407.1 (MH⁺).

7-(1-ethyl-1H-pyrrolo[2,3-b]pyridin-5-yl)-4-quinolin-4-yl-2,3,4,5-tetrahydro-1,4-benzoxazepine. Synthesized according to the method of example 3 using 5-bromo-1-ethyl-1H-indole (reagent preparation 21) in step 1 and 4-chloroquinoline in step 3. ¹H NMR (400 MHz, DMSO-d₆): 8.62 (d, 1H), 8.57 (d, 1H), 8.22 (d, 1H), 8.01 (d, 1H), 7.94 (d, 1H), 7.74 (d, 1H), 7.69 (t, 1H), 7.64 (dd, 1H), 7.61 (dd, 1H), 7.50 (t, 1H), 7.12 (d, 1H), 7.03 (d, 1H), 6.52 (d, 1H), 4.66 (s, 2H), 4.42 (m, 2H), 4.31 (qr. 2H), 3.85 (s, 3H), 3.82 (m, 2H), 1.39 (t, 3H); MS (EI) for C₂₇H₂₄N₄O₂: 421.2 (MH⁺).

5-(4-quinolin-4-yl-2,3,4,5-tetrahydro-1,4-benzoxazepin-7-yl)pyridin-2-amine. Synthesized according to the method of example 3 using 5-bromopyridin-2-amine in step 1 and 4-chloroquinoline in step 3. ¹H NMR (400 MHz, DMSO-d₆): 8.61 (d, 1H), 8.24 (d, 1H), 7.99 (d, 1H), 7.93 (d, 1H), 7.71-7.69 (m, 2H), 7.59 (m, 1H), 7.50 (m, 1H), 7.44 (dd, 1H), 7.04 (d, 1H), 7.00 (dd, 1H), 6.52 (d, 1H), 6.04 (s, 2H), 4.61 (s, 2H), 4.34 (m, 2H), 3.80 (m, 2H), 1.88 (s, 3H, AcOH); MS (EI) for C₂₃H₂₀N₄O₂: 369.13 (MH⁺).

4-[6,7-bis(methyloxy)quinolin-4-yl]-7-[4-(1H-imidazol-2-yl)phenyl]-2,3,4,5-tetrahydro-1,4-benzoxazepine. Synthesized according to the method of example 3 using 2-(4-bromophenyl)-1H-imidazole in step 1 and 4-chloro-6,7-dimethoxyquinoline in step 3. ¹H NMR (400 MHz, DMSO-d₆): 8.47 (d, 1H), 8.01 (d, 2H), 7.87 (d, 1H), 7.77 (d, 2H), 7.65 (dd, 1H), 7.31 (s, 1H), 7.16 (bs, 2H), 7.12 (d, 1H), 7.10 (s, 1H), 6.94 (d, 1H), 4.65 (s, 2H), 4.44 (m, 2H), 3.89 (s, 3H), 3.77 (m, 2H), 3.54 (s, 3H); MS (EI) for C₂₉H₂₆N₄O₃: 478.9 (MH⁺).

7-[4-(1H-imidazol-2-yl)phenyl]-4-(2-methylquinazolin-4-yl)-2,3,4,5-tetrahydro-1,4-benzoxazepine. Synthesized according to the method of example 3 using 2-(4-bromophenyl)-1H-imidazole in step 1 and 4-chloro-2-methylquinazoline in step 3. ¹H NMR (400 MHz, DMSO-d₆): 12.5 (s, 1H), 8.02 (d, 2H), 7.99 (d, 1H), 7.78-7.74 (m, 4H), 7.70 (m, 1H), 7.59 (m, 1H), 7.43 (m, 1H), 7.26 (bs, 1H), 7.03 (d, 2H), 5.06 (s, 2H), 4.74 (s, 2H), 4.19 (s, 2H), 2.46 (s, 3H); MS (EI) for C₂₇H₂₃N₅O: 434.0 (MH⁺).

5-[4-(2-methylquinazolin-4-yl)-2,3,4,5-tetrahydro-1,4-benzoxazepin-7-yl]pyridin-2-amine. Synthesized according to the method of example 3 using 5-bromo-2-aminopyridine in step 1 and 4-chloro-2-methylquinazoline in step 3. ¹H NMR (400 MHz, DMSO-d₆): 8.21 (m, 1H), 7.97 (dd, 1H), 7.75 (m, 1H), 7.69 (m, 1H), 7.66 (m, 1H), 7.57 (m, 1H), 7.40 (m, 2H), 6.97 (d, 1H), 6.52 (d, 1H), 6.03 (s, 2H), 5.00 (s, 2H), 4.42 (s, 2H), 4.17 (s, 2H), 2.46 (s, 3H); MS (EI) for C₂₃H₂₁N₅O: 384.2 (MH⁺).

4-[2-methyl-7-(methyloxy)quinazolin-4-yl]-7-(1H-pyrrolo[2,3-b]pyridin-5-yl)-2,3,4,5-tetrahydro-1,4-benzoxazepine. Synthesized according to the method of example 3 using 5-bromo-1H-pyrrolo[2,3-b]pyridine in step 1 and 4-chloro-7-methoxy-2-methylquinazoline in step 3. ¹H NMR (400 MHz, DMSO-d₆): 11.7 (s, 1H), 8.49 (s, 1H), 8.16 (s, 1H), 7.90 (d, 1H), 7.71 (s, 1H), 7.53 (m, 2H), 7.11 (s, 1H), 7.03 (d, 2H), 6.50 (m, 1H), 5.02 (s, 2H), 4.44 (s, 2H), 4.15 (d, 2H), 3.87 (s, 3H), 2.43 (s, 3H); MS (EI) for C₂₆H₂₃N₅O₂: 438.2 (MH⁺).

(5-{4-[2-methyl-7-(methyloxy)quinazolin-4-yl]-2,3,4,5-tetrahydro-1,4-benzoxazepin-7-yl}pyridin-2-amine. Synthesized according to the method of example 3 using 5-bromo-2-aminopyridine in step 1 and 4-chloro-7-methoxy-2-methylquinazoline in step 3. ¹H NMR (400 MHz, DMSO-d₆): 11.9 (s, 1H, AcOH), 8.21 (d, 1H), 7.87 (d, 1H), 7.67 (dd, 1H), 7.55 (m, 1H), 7.38 (dd, 1H), 7.10 (d, 1H), 7.02 (dd, 1H), 6.96 (d, 1H), 6.52 (d, 1H), 6.03 (s, 2H), 4.96 (s, 2H), 4.38 (m, 2H), 4.13 (m, 2H), 3.87 (s, 3H), 2.42 (s, 3H), 1.90 (s, 3H, AcOH); MS (EI) for C₂₄H₂₃N₅O₂: 413.9 (MH⁺).

7-(1H-indazol-5-yl)-4-[2-methyl-7-(methyloxy)quinazolin-4-yl]-2,3,4,5-tetrahydro-1,4-benzoxazepine. Synthesized according to the method of example 3 using 5-bromo-1H-pyrazolo[3,4-b]pyridine in step 1 and 4-chloro-7-methoxy-2-methylquinazoline in step 3. ¹H NMR (400 MHz, DMSO-d₆): 13.1 (s, 1H), 11.9 (s, 1H, AcOH), 8.12 (s, 1H), 7.99 (m, 1H), 7.92 (d, 1H), 7.69 (m, 1H), 7.64 (m, 2H), 7.53 (d, 1H), 7.10 (d, 1H), 7.05 (dd, 1H), 7.02 (dd, 1H), 5.04 (s, 2H), 4.45 (m, 2H), 4.17 (bs, 2H), 3.88 (s, 3H), 2.43 (s, 3H), 1.90 (s, 3H, AcOH); MS (EI) for C₂₆H₂₃N₅O₂: 437.9 (MH⁺).

5-{4-[2-methyl-7-(methyloxy)quinazolin-4-yl]-2,3,4,5-tetrahydro-1,4-benzoxazepin-7-yl}pyrimidin-2-amine. Synthesized according to the method of example 3 using 5-bromo-2-aminopyrimidine in step 1 and 4-chloro-7-methoxy-2-methylquinazoline in step 3. ¹H NMR (400 MHz, DMSO-d₆): 8.55 (s, 2H), 7.86 (d, 1H), 7.62 (d, 1H), 7.45 (dd, 2H), 7.10 (d, 1H), 7.03 (dd, 1H), 6.99 (d, 1H), 6.75 (s, 2H), 4.97 (s, 2H), 4.42 (bs, 2H), 4.12 (bs, 2H), 3.87 (s, 3H), 2.42 (s, 3H); MS (EI) for C₂₃H₂₂N₆O₂: 415.0 (MH⁺).

5-{4-[2-methyl-7-(methyloxy)quinazolin-4-yl]-2,3,4,5-tetrahydro-1,4-benzoxazepin-7-yl}-1,3-thiazol-2-amine. Synthesized according to the method of example 3 using 4-chloro-7-methoxy-2-methylquinazoline in step 3. ¹H NMR (400 MHz, DMSO-d₆): 7.86 (d, 1H), 7.39 (d, 1H), 7.30 (s, 1H), 7.26 (dd, 1H), 7.10 (m, 3H), 7.01 (dd, 1H), 6.90 (d, 1H), 4.90 (s, 2H), 4.36 (m, 2H), 4.12 (m, 2H), 3.87 (s, 3H), 2.42 (s, 3H); MS (EI) for C₂₂H₂₁N₅O₂S: 419.9 (MH⁺).

N-(5-{4-[2-methyl-7-(methyloxy)quinazolin-4-yl]-2,3,4,5-tetrahydro-1,4-benzoxazepin-7-yl}-1,3-thiazol-2-yl)acetamide. Synthesized according to the method of example 3 using 4-chloro-7-methoxy-2-methylquinazoline in step 3. ¹H NMR (400 MHz, DMSO-d₆): 7.86 (d, 1H), 7.78 (s, 1H), 7.63 (d, 1H), 7.46 (dd, 1H), 7.10 (d, 1H), 6.98 (dd, 1H), 6.57 (d, 1H), 4.96 (s, 2H), 4.41 (m, 2H), 4.14 (m, 2H), 3.87 (s, 3H), 2.41 (s, 3H), 2.16 (s, 3H); MS (EI) for C₂₄H₂₃N₅O₃S: 462.1 (MH⁺).

7-(1,3-benzothiazol-6-yl)-4-[2-methyl-7-(methyloxy)quinazolin-4-yl]-2,3,4,5-tetrahydro-1,4-benzoxazepine. Synthesized according to the method of example 3 using 6-bromobenzo[d]thiazole in step 1 and 4-chloro-7-methoxy-2-methylquinazoline in step 3. ¹H NMR (400 MHz, DMSO-d₆): 9.40 (s, 1H), 8.46 (d, 1H), 8.16 (dd, 1H), 7.88 (d, 1H), 7.80 (dd, 1H), 7.78 (d, 1H), 7.61 (dd, 1H), 7.10 (d, 1H), 7.05 (m, 2H), 5.03 (s, 2H), 4.47 (m, 2H), 4.15 (m, 2H), 3.87 (s, 3H), 2.42 (s, 3H), 1.90 (s, 3H, AcOH); MS (EI) for C₂₆H₂₂N₄O₂S: 455.1 (MH⁺).

5-{4-[2-methyl-7-(methyloxy)quinazolin-4-yl]-2,3,4,5-tetrahydro-1,4-benzoxazepin-7-yl}-1,3-dihydro-2H-benzimidazol-2-one. Synthesized according to the method of example 3 using 5-bromo-1H-benzo[d]imidazol-2(3H)-one in step 1 and 4-chloro-7-methoxy-2-methylquinazoline in step 3. ¹H NMR (400 MHz, DMSO-d₆): 10.7 (d, 2H), 7.90 (d, 1H), 7.58 (s, 1H), 7.24 (d, 1H), 7.14 (d, 1H), 7.04 (d, 2H), 7.01 (m, 3H), 4.98 (s, 2H), 4.42 (s, 2H), 4.14 (s, 2H), 2.43 (s, 3H); MS (EI) for C₂₆H₂₃N₅O₃: 453.9 (MH⁺).

5-{4-[2-methyl-7-(methyloxy)quinazolin-4-yl]-2,3,4,5-tetrahydro-1,4-benzoxazepin-7-yl}-1,3-dihydro-2H-indol-2-one. Synthesized according to the method of example 3 using 5-bromoindolin-2-one in step 1 and 4-chloro-7-methoxy-2-methylquinazoline in step 3. ¹H NMR (400 MHz, DMSO-d₆): 10.4 (s, 1H), 7.87 (d, 1H), 7.58 (s, 1H), 7.48 (s, 1H), 7.43 (t, 2H), 7.10 (s, 1H), 6.99 (dd, 1H), 6.97 (d, 1H), 6.89 (d, 1H), 4.98 (s, 2H), 4.42 (s, 2H), 4.13 (s, 2H), 3.89 (s, 3H), 3.53 (s, 2H), 2.42 (s, 2H); MS (EI) for C₂₇H₂₄N₄O₃: 452.9 (MH⁺).

N-[6-(4-{2-[(dimethylamino)methyl]-6,6-dimethyl-5,6,7,8-tetrahydroquinazolin-4-yl}-2,3,4,5-tetrahydro-1,4-benzoxazepin-7-yl)[1,3]thiazolo[5,4-b]pyridin-2-yl]acetamide. Prepared according to the method of example 3 by using N-(6-bromo[1,3]thiazolo[5,4-b]pyridin-2-yl)acetamide (Journal of Heterocyclic Chemistry (200), 40(2), 621-628) in step 1 and 1-(4-chloro-6,6-dimethyl-5,6,7,8-tetrahydroquinazolin-2-yl)-N,N-dimethylmethanamine (reagent preparation 17) in step 3. MS (EI) for C₃₀H₃₅N₇O₂S 558 (MH⁺).

6-(4-{2-[(dimethylamino)methyl]-6,6-dimethyl-5,6,7,8-tetrahydroquinazolin-4-yl}-2,3,4,5-tetrahydro-1,4-benzoxazepin-7-yl)[1,3]thiazolo[5,4-b]pyridin-2-amine. Prepared according to the method of example 3 by using N-(6-bromo[1,3]thiazolo[5,4-b]pyridin-2-yl)acetamide (Journal of Heterocyclic Chemistry (200), 40(2), 621-628) in step 1 and 1-(4-chloro-6,6-dimethyl-5,6,7,8-tetrahydroquinazolin-2-yl)-N,N-dimethylmethanamine (reagent preparation 17) in step 3. ¹H NMR (400 MHz, d₆-DMSO): 8.40 (d, 1H), 7.90 (s, 2H), 7.84 (d, 1H), 7.74 (d, 1H), 7.54 (dd, 1H), 7.01 (d, 1H), 4.67 (s, 2H), 4.32 (s, 2H), 3.89 (s, 2H), 3.43 (s, 2H), 2.68 (m, 2H), 2.46 (m, 2H), 2.20 (s, 6H), 1.60 (m, 2H), 0.84 (s, 6H); MS (EI) for C₂₈H₃₃N₇OS: 516 (MH⁺).

1-(4-{7-[2-(fluoromethyl)-1H-benzimidazol-5-yl]-2,3-dihydro-1,4-benzoxazepin-4(5H)-yl}-6,6-dimethyl-5,6,7,8-tetrahydroquinazolin-2-yl)-N,N-dimethylmethanamine. Prepared according to the method of example 3 by using 1,1-dimethylethyl 5-bromo-2-(fluoromethyl)-1H-benzimidazole-1-carboxylate (reagent preparation 19) in step 1, and 1-(4-chloro-6,6-dimethyl-5,6,7,8-tetrahydroquinazolin-2-yl)-N,N-dimethylmethanamine (reagent preparation 17) in step 3. ¹H NMR (400 MHz, Methanol-D₄): 7.79 (br, 1H), 7.67 to 7.60 (m, 2H), 7.56 (d, 1H), 7.50 (d, 1H), 7.04 (d, 1H), 5.62 (d, 2H), 4.79 (s, 2H), 4.37 (m, 2H), 4.01 (m, 2H), 3.91 (s, 2H), 2.79 (t, 2H), 2.59 (s, 6H), 2.52 (s, 2H), 1.70 (t, 2H), 0.92 (s, 6H); MS (EI) for C₃₀H₃₅FN₆O: 515 (MH⁺).

1-(4-{7-[2-(fluoromethyl)-1H-benzimidazol-5-yl]-2,3-dihydro-1,4-benzoxazepin-4(5H)-yl}-5-[(4-fluorophenyl)methyl]-6-methylpyrimidin-2-yl)-N,N-dimethylmethanamine. Prepared according to the method of example 3 by using 1,1-dimethylethyl 5-bromo-2-(fluoromethyl)-1H-benzimidazole-1-carboxylate (reagent preparation 19) in step 1, and 1-{4-chloro-5-[(4-fluorophenyl)methyl]-6-methylpyrimidin-2-yl}-N,N-dimethylmethanamine (reagent preparation 17) in step 3. ¹H NMR (400 MHz, Methanol-D₄): 7.66 (br, 2H), 7.46 (d, 1H), 7.38 (d, 1H), 7.09 (m, 2H), 7.01 to 6.93 (m, 4H), 5.63 (d, 2H), 4.77 (s, 2H), 4.36 (m, 2H), 4.02 (m, 2H), 3.91 (br, 4H), 2.59 (s, 2H), 2.26 (s, 3H); MS (EI) for C₃₂H₃₂FN₆O: 555 (MH⁺).

1-[4-{7-[2-(fluoromethyl)-1H-benzimidazol-5-yl]-2,3-dihydro-1,4-benzoxazepin-4(5H)-yl}-6-methyl-5-(1-methylethyl)pyrimidin-2-yl]-N,N-dimethylmethanamine. Prepared according to the method of example 3 using 1,1-dimethylethyl 5-bromo-2-(fluoromethyl)-1H-benzimidazole-1-carboxylate (reagent preparation 19) in step 1, and 1-[4-chloro-6-methyl-5-(1-methylethyl)pyrimidin-2-yl]-N,N-dimethylmethanamine (reagent preparation 17) in step 3. ¹H NMR (400 MHz, Methanol-D₄): 7.78 (br, 1H), 7.65 (br, 1H), 7.58 to 7.50 (m, 3H), 7.04 (d, 1H), 5.62 (d, 2H), 4.62 (s, 2H), 4.37 (m, 2H), 3.86 (m, 2H), 3.37 (m, 1H), 2.56 (s, 3H), 2.54 (s, 3H), 1.41 (d, 6H); MS (EI) for C₁₋₂₈H₃₃FN₆O: 489 (MH⁺).

1-{4-[7-(4-fluoro-2-methyl-1H-benzimidazol-5-yl)-2,3-dihydro-1,4-benzoxazepin-4(5H)-yl]-6,6-dimethyl-5,6,7,8-tetrahydroquinazolin-2-yl}-N,N-dimethylmethanamine. Prepared according to the method of example 3 by using 1,1-dimethylethyl 5-bromo-4-fluoro-2-methyl-1H-benzimidazole-1-carboxylate (reagent preparation 19) in step 1, and 1-(4-chloro-6,6-dimethyl-5,6,7,8-tetrahydroquinazolin-2-yl)-N,N-dimethylmethanamine (reagent preparation 17) in step 3. ¹H NMR (400 MHz, Methanol-D₄): 7.49 (br, 1H), 7.59 (d, 1H), 7.36 (d, 1H), 7.28 (d, 1H), 7.01 (d, 1H), 4.75 (s, 2H), 4.36 (m, 2H), 4.00 (m, 2H), 3.80 (s, 2H), 2.79 (t, 2H), 2.56 (s, 3H), 2.49 (br, 6H), 1.65 (t, 2H), 0.91 (s, 6H); MS (EI) for C₃₀H₃₅FN₆O: 515 (MH⁺).

4-(6,6-dimethyl-5,6-dihydroquinazolin-4-yl)-7-[4-(1H-imidazol-2-yl)phenyl]-2,3,4,5-tetrahydro-1,4-benzoxazepine. Prepared according to the method of example 3 by using isobutyl 2-(4-bromophenyl)-1H-imidazole-1-carboxylate (reagent preparation 11) in step 1 and 4-chloro-6,6-dimethyl-5,6-dihydroquinazoline (reagent preparation 3) in step 3. ¹H NMR (400 MHz, methanol-d₄): 8.38 (s, 1H), 7.92 (m, 2H), 7.70 (m, 2H), 7.59 (m, 1H), 7.53 (m, 1H), 7.15 (brs, 2H), 7.07 (d, 1H), 6.33 (d, 1H), 6.26 (d, 1H), 4.71 (s, 2H), 4.36 (m, 2H), 3.97 (m, 2H), 2.78 (s, 2H), 1.01 (s, 6H); MS (EI) for C₂₈H₂₇N₅O: 450 (MH⁺).

1-(4-{7-[4-(1H-imidazol-2-yl)phenyl]-2,3-dihydro-1,4-benzoxazepin-4(5H)-yl}-6,6-dimethyl-5,6,7,8-tetrahydroquinazolin-2-yl)-N,N-dimethylmethanamine. Prepared as dihydrochloride salt according to the method of example 3 by using isobutyl 2-(4-bromophenyl)-1H-imidazole-1-carboxylate (reagent preparation 11) in step 1 and 1-(4-chloro-6,6-dimethyl-5,6,7,8-tetrahydroquinazolin-2-yl)-N,N-dimethylmethanamine (reagent preparation 17) in step 3. ¹H NMR (400 MHz, methanol-d₄): 8.00 (m, 4H), 7.85 (s, 1H), 7.68 (s, 2H), 7.64 (dd, 1H), 7.09 (d, 1H), 5.04 (s, 2H), 4.52 (m, 2H), 4.42 (s, 2H), 4.18 (m, 2H), 2.93 (s, 6H), 2.85 (t, 2H), 2.55 (s, 2H), 1.70 (t, 2H), 0.89 (s, 6H); MS (EI) for C₃₁H₃₆N₆O: 509 (MH⁺).

7-[4-(1H-imidazol-2-yl)phenyl]-4-[2-methyl-7-(methyloxy)quinazolin-4-yl]-2,3,4,5-tetrahydro-1,4-benzoxazepine. Prepared according to the method of example 3 by using 2-(4-bromophenyl)-1H-imidazole in step 1 and 4-chloro-7-methoxy-2-methylquinazoline in step 3. ¹H NMR (400 MHz, DMSO-d6); δ 11.99 (s, 1H), 8.10-7.94 (m, 3H), 7.86-7.75 (m, 3H), 7.65-7.57 (m, 3H), 7.27 (s, 1H), 7.15-7.09 (m, 2H), 7.06-6.99 (d, 1H), 5.15 (s, 2H), 4.55-4.47 (m, 2H), 4.30-4.22 (m, 2H), 3.90 (3H), 2.46 (s, 3H); MS (EI) for C₂₈H₂₅N₅O₂: 464 (MH⁺).

7-[4-(1H-imidazol-4-yl)phenyl]-4-[2-methyl-7-(methyloxy)quinazolin-4-yl]-2,3,4,5-tetrahydro-1,4-benzoxazepine. Prepared according to the method of example 3 by using 4-(4-bromophenyl)-1H-imidazole in step 1 and 4-chloro-7-methoxy-2-methylquinazoline in step 3. ¹H NMR (400 MHz, DMSO-d6); δ 12.2 (s, 1H), 7.93-7.84 (m, 3H), 7.78-7.61 (m, 5H), 7.57-7.50 (m, 1H), 7.11 (d, 1H), 7.05-7.00 (m, 2H), 5.01 (s, 2H), 4.48-4.40 (m, 2H), 4.18-4.11 (m, 2H), 3.88 (s, 3H), 2.43 (s, 3H); MS (EI) for C₂₈H₂₅N₅O₂: 464 (MH⁺).

7-[4-(1H-benzimidazol-2-yl)phenyl]-4-[2-methyl-7-(methyloxy)quinazolin-4-yl]-2,3,4,5-tetrahydro-1,4-benzoxazepine. Prepared according to the method of example 3 by using 2-(4-bromophenyl)-1H-benzo[d]imidazole in step 1 and 4-chloro-7-methoxy-2-methylquinazoline in step 3. ¹H NMR (400 MHz, DMSO-d6); δ 8.36-7.85 (m, 7H), 7.73-7.59 (m, 3H), 7.33-7.20 (m, 3H), 7.15-7.11 (m, 1H), 7.07-6.99 (m, 1H), 5.39 (s, 2H), 4.69-4.58 (m, 2H), 4.53-4.40 (m, 2H), 3.95 (s, 3H), 2.54 (s, 3H); MS (EI) for C₃₂H₂₇N₅O₂: 514 (MH⁺).

4-{4-[2-methyl-7-(methyloxy)quinazolin-4-yl]-2,3,4,5-tetrahydro-1,4-benzoxazepin-7-yl}aniline. Prepared according to the method of example 3 by using 4-bromoaniline in step 1 and 4-chloro-7-methoxy-2-methylquinazoline in step 3. ¹H NMR (400 MHz, DMSO-d6); δ 7.89 (d, 1H), 7.51-7.47 (m, 1H), 7.37-7.28 (m, 3H), 7.11-7.08 (m, 1H), 7.04-6.99 (m, 1H), 6.96-6.92 (m, 1H), 6.65-6.60 (m, 2H), 5.19 (s, 2H), 4.95 (s, 2H), 4.41-4.35 (m, 2H), 4.15-4.09 (m, 2H), 3.88 (s, 3H), 2.43 (s, 3H); MS (EI) for C₂₅H₂₄N₄O₂: 413 (MH⁺).

{5-[4-(4-pyrido[3,2-d]pyrimidin-4-yl-2,3,4,5-tetrahydro-1,4-benzoxazepin-7-yl)phenyl]-1H-imidazol-2-yl}methanol. Prepared according to the method of example 3 by using (4-(4-bromophenyl)-1H-imidazol-2-yl)methanol (reagent preparation 22) in step 1 and 4-chloropyrido[3,2-d]pyrimidine in step 3. ¹H NMR (400 MHz, DMSO-d6) δ 12.03 (br s, 1H), 9.01 (d, 1H), 8.57 (s, 1H), 8.50 (d, 1H), 7.80 (m, 8H), 7.02 (d, 1H), 5.50 (br s, 1H), 5.20 (s, 2H), 4.52 (s, 4H), 4.34 (s, 2H); MS (EI for C₂₆H₂₂N₆O₂: 451.1 (MH⁺)

7-(2,4-dimethyl-1H-benzimidazol-6-yl)-4-pyrido[3,2-d]pyrimidin-4-yl-2,3,4,5-tetrahydro-1,4-benzoxazepine. Prepared according to the method of example 3 by using tert-butyl 6-bromo-2,4-dimethyl-1H-benzo[d]imidazole-1-carboxylate (reagent preparation 19) in step 1 and 4-chloropyrido[3,2-d]pyrimidine in step 3. ¹H NMR (400 MHz, DMSO-d6) δ 12.03 (br s, 1H), 9.01 (d, 1H), 8.57 (s, 1H), 8.50 (d, 1H), 7.70 (s, 1H), 7.50 (m, 3H), 7.25 (s, 1H), 6.98 (d, 1H), 5.20 (s, 2H), 4.51 (s, 2H), 4.25 (s, 2H), 3.31 (s, 3H), 2.51 (s, 3H); MS (EI) for C₂₅H₂₂N₆O: 423.1 (MH⁺).

1-(4-{7-[3,4-bis(methyloxy)phenyl]-2,3-dihydro-1,4-benzoxazepin-4(5H)-yl}-6,6-dimethyl-5,6,7,8-tetrahydroquinazolin-2-yl)-N,N-dimethylmethanamine. Prepared according to the method of example 3 by using 4-bromo-1,2-dimethoxybenzene in step 1 and 1-(4-chloro-6,6-dimethyl-5,6,7,8-tetrahydroquinazolin-2-yl)-N,N-dimethylmethanamine (reagent preparation 17) in step 3. ¹H NMR (400 MHz, DMSO-d₆): 7.62 (d, 1H), 7.47-7.42 (m, 1H), 7.19-7.13 (m, 2H), 7.04-6.96 (m, 2H), 4.61 (s, 2H), 4.28 (t, 2H), 3.87-3.81 (m, 5H), 3.78 (s, 3H), 3.37 (s, 2H), 2.69 (t, 2H), 2.45 (s, 2H), 2.15 (s, 6H), 1.59 (t, 2H), 0.86 (s, 6H); MS (EI) for C₃₀H₃₈N₄O₃: 503 (MH⁺).

1-(6,6-dimethyl-4-{7-[6-(methyloxy)pyridin-3-yl]-2,3-dihydro-1,4-benzoxazepin-4(5H)-yl}-5,6,7,8-tetrahydroquinazolin-2-yl)-N,N-dimethylmethanamine. Prepared according to the method of example 3 by using 5-bromo-2-methoxypyridine in step 1 and 1-(4-chloro-6,6-dimethyl-5,6,7,8-tetrahydroquinazolin-2-yl)-N,N-dimethylmethanamine (reagent preparation 17) in step 3. ¹H NMR (400 MHz, DMSO-d₆): 8.45 (d, 1H), 8.00-7.95 (m, 1H), 7.65 (d, 1H), 7.46 (m, 1H), 7.00 (d, 1H), 6.91 (d, 1H), 4.62 (s, 2H), 4.30 (m, 2H), 3.91-3.83 (m, 5H), 3.36 (m, 2H), 2.69 (t, 2H), 2.43 (s, 2H), 2.13 (s, 6H), 1.59 (t, 2H), 0.85 (s, 6H); MS (EI) for C₂₈H₃₅N₅O₂: 474 (MH⁺).

1-(6,6-dimethyl-4-{7-[3-(methyloxy)phenyl]-2,3-dihydro-1,4-benzoxazepin-4(5H)-yl}-5,6,7,8-tetrahydroquinazolin-2-yl)-N,N-dimethylmethanamine. Prepared according to the method of example 3 by using 1-bromo-3-methoxybenzene in step 1 and 1-(4-chloro-6,6-dimethyl-5,6,7,8-tetrahydroquinazolin-2-yl)-N,N-dimethylmethanamine (reagent preparation 17) in step 3. ¹H NMR (400 MHz, DMSO-d₆): 7.66 (d, 1H), 7.50-7.45 (m, 1H), 7.36 (t, 1H), 7.22-7.18 (m, 1H), 7.15 (m, 1H), 7.00 (d, 1H), 6.93-6.88 (m, 1H), 4.62 (s, 2H), 4.29 (t, 2H), 3.85 (t, 2H), 3.81 (s, 3H), 2.68 (t, 2H), 2.44 (s, 2H), 2.12 (s, 6H), 1.59 (t, 2H), 0.85 (s, 6H); MS (EI) for C₂₉H₃₆N₄O₂: 473 (MH⁺).

1-[4-{7-[3,4-bis(methyloxy)phenyl]-2,3-dihydro-1,4-benzoxazepin-4(5H)-yl}-7-(methyloxy)quinazolin-2-yl]-N,N-dimethylmethanamine. Prepared according to the method of example 3 using 4-bromo-1,2-dimethoxybenzene in step 1 and 1-(4-chloro-7-methoxyquinazolin-2-yl)-N,N-dimethylmethanamine (reagent preparation 17) in step 3. ¹H NMR (400 MHz, DMSO-d₆): 7.95 (d, 1H), 7.64 (d, 1H), 7.47-7.43 (m, 1H), 7.20-7.14 (m, 3H), 7.09-7.01 (m, 2H), 6.95 (d, 1H), 5.04 (s, 2H), 4.44 (m, 2H), 4.19 (m, 2H), 3.89 (s, 3H), 3.85 (s, 3H), 3.79 (s, 3H), 3.48 (s, 2H), 2.17 (s, 6H); MS (EI) for C₂₉H₃₂N₄O₄: 501 (MH⁺).

1-{4-[7-(1,3-benzothiazol-5-yl)-2,3-dihydro-1,4-benzoxazepin-4(5H)-yl]-5-[(4-fluorophenyl)methyl]-6-methylpyrimidin-2-yl}-N,N-dimethylmethanamine. Synthesized according to the method of example 3 using 5-bromobenzo[d]thiazole in step 1. ¹H NMR (400 MHz, d₆-DMSO): 9.47 (s, 1H), 8.25 (d, 1H), 8.23 (d, 1H), 7.64 (m, 2H), 7.14 (d, 4H), 7.10 (m, 1H), 7.03 (d, 1H), 4.64 (s, 2H), 4.38-4.34 (m, 4H), 4.01 (s, 2H), 3.87 (m, 2H); MS (EI) for C₃₁H₃₀FN₅OS: 540 (MH⁺).

1-{4-[7-(1,3-benzothiazol-5-yl)-2,3-dihydro-1,4-benzoxazepin-4(5H)-yl]-6,6-dimethyl-5,6,7,8-tetrahydroquinazolin-2-yl}-N,N-dimethylmethanamine. Synthesized according to the method of example 3 using 5-bromobenzo[d]thiazole in step 1 and 1-(4-chloro-6,6-dimethyl-5,6,7,8-tetrahydroquinazolin-2-yl)-N,N-dimethylmethanamine (reagent preparation 17) in step 3. ¹H NMR (400 MHz, d₆-DMSO): 9.44 (s, 1H), 8.37 (d, 1H), 8.25 (d, 1H), 7.86 (d, 1H), 7.80 (dd, 1H), 7.64 (dd, 1H), 7.05 (d, 1H), 4.81 (s, 2H), 4.42 (m, 2H), 4.31 (s, 2H), 3.97 (br t, 2H), 2.80 (s, 6H), 2.75 (t, 2H), 1.62 (t, 2H), 0.86 (s, 6H); MS (EI) for C₂₉H₃₃N₅OS: 500 (MH⁺).

1-{4-[7-(6-fluoro-2-methyl-1H-benzimidazol-5-yl)-2,3-dihydro-1,4-benzoxazepin-4(5H)-yl]-6,6-dimethyl-5,6,7,8-tetrahydroquinazolin-2-yl}-N,N-dimethylmethanamine. Synthesized according to the method of example 3 using 5-bromo-6-fluoro-2-methyl-1H-benzo[d]imidazole (reagent preparation 19) in step 1 and 1-(4-chloro-6,6-dimethyl-5,6,7,8-tetrahydroquinazolin-2-yl)-N,N-dimethylmethanamine (reagent preparation 17) in step 3. ¹H NMR (400 MHz, d₆-DMSO) δ 7.88-7.78 (m, 2H), 7.69 (s, 1H), 7.42 (d, 1H), 7.06 (d, 1H), 4.90 (s, 2H), 4.48-4.39 (m, 2H), 4.33 (s, 2H), 4.06-3.99 (m, 2H), 2.80 (s, 3H), 2.79 (s, 6H), 2.75 (t, 2H), 2.48 (s, 2H), 1.59 (t, 2H), 0.85 (s, 6H); MS (EI) for C₃₀H₃₅FN₆O: 515 (MH⁺).

1-(4-{7-[2-(difluoromethyl)-1H-benzimidazol-5-yl]-2,3-dihydro-1,4-benzoxazepin-4(5H)-yl}-5-[(4-fluorophenyl)methyl]-6-methylpyrimidin-2-yl)-N,N-dimethylmethanamine. Synthesized according to the method of example 3 using tert-butyl 6-bromo-2-(difluoromethyl)-1H-benzo[d]imidazole-1-carboxylate (reagent preparation 19) in step 1 and 1-(4-chloro-5-(4-fluorobenzyl)-6-methylpyrimidin-2-yl)-N,N-dimethylmethanamine (reagent preparation 17) in step 3. ¹H NMR (400 MHZ, CDCl₃): 7.50-7.26 (br, 2H), 7.33 (dd, 1H), 7.25 (dd, 1H), 6.99 (d, 1H), 6.95 (tr, CHF₂, 1H), 6.92-6.81 (m, 5H), 6.54 (br s, 1H), 4.10 (br s, 4H), 3.81 (s, 2H), 3.70 (s, 2H), 3.61 (tr, 2H), 2.47 (s, 6H), 2.17 (s, 3H). MS (EI) for C₃₂H₃₁N₆OF₃: 573 (MH⁺).

1-[4-{7-[2-(difluoromethyl)-1H-benzimidazol-5-yl]-2,3-dihydro-1,4-benzoxazepin-4(5H)-yl}-6-methyl-5-(1-methylethyl)pyrimidin-2-yl]-N,N-dimethylmethanamine. Synthesized according to the method of example 3 using tert-butyl 6-bromo-2-(difluoromethyl)-1H-benzo[d]imidazole-1-carboxylate (reagent preparation 19) in step 1 and 1-(4-chloro-5-isopropyl-6-methylpyrimidin-2-yl)-N,N-dimethylmethanamine (reagent preparation 17) in step 3. ¹H NMR (400 MHZ, CDCl₃): 7.50-7.26 (br, 2H), 7.38-7.32 (br m, 2H), 7.15 (br, 1H), 7.03 (d, 1H), 6.94 (tr, CHF₂, 1H), 4.13 (br s, 2H), 3.66 (s, 2H), 3.43 (br, 2H), 3.24 (m, 1H), 2.50 (s, 6H), 2.47 (s, 3H), 1.21 (d, 6H). MS (EI) for C₂₈H₃₂N₆OF₂: 507 (MH⁺).

1-(4-{7-[2-(difluoromethyl)-1H-benzimidazol-5-yl]-2,3-dihydro-1,4-benzoxazepin-4(5H)-yl}-6,6-dimethyl-5,6,7,8-tetrahydroquinazolin-2-yl)-N,N-dimethylmethanamine. Synthesized according to the method of example 3 using tert-butyl 6-bromo-2-(difluoromethyl)-1H-benzo[d]imidazole-1-carboxylate (reagent preparation 19) in step 1 and 1-(4-chloro-6,6-dimethyl-5,6,7,8-tetrahydroquinazolin-2-yl)-N,N-dimethylmethanamine (reagent preparation 17) in step 3. ¹H NMR (400 MHZ, CDCl₃): 7.50-7.26 (br, 2H), 7.36 (br d, 1H), 7.31 (br dd, 1H), 7.20 (br, 1H), 7.02 (d, 1H), 6.97 (tr, CHF₂, 1H), 4.16 (tr, 2H), 4.07 (br, 2H), 3.65 (s, 2H), 3.60 (br s, 2H), 2.73 (tr, 2H), 2.47 (s, 6H), 2.22 (br s, 2H), 1.50 (tr, 2H), 0.86 (s, 6H). MS (EI) for C₃₀H₃₄N₆OF₂: 533 (MH⁺).

1-(6,6-dimethyl-4-{7-[3-(methyloxy)-4-{[2-(methyloxy)ethyl]oxy}-phenyl]-2,3-dihydro-1,4-benzoxazepin-4(5H)-yl}-5,6,7,8-tetrahydroquinazolin-2-yl)-N,N-dimethylmethanamine. Prepared according to the method of example 3 by using 4-bromo-2-methoxy-1-(2-methoxyethoxy)benzene in step 1 and 1-(4-chloro-6,6-dimethyl-5,6,7,8-tetrahydroquinazolin-2-yl)-N,N-dimethylmethanamine (reagent preparation 17) in step 3. ¹H NMR (400 MHz, (DMSO-d₆): 7.62 (d, 1H), 7.47-7.43 (m, 1H), 7.18 (d, 1H), 7.15-7.11 (m, 1H), 7.04-6.95 (m, 2H), 4.60 (s, 2H), 4.28 (m, 2H), 4.10 (m, 2H), 3.87-3.82 (m, 5H), 3.67 (m, 2H), 3.36 (m, 2H), 3.32 (s, 3H), 2.69 (t, 2H), 2.45 (s, 2H), 2.14 (s, 6H), 1.59 (t, 2H), 0.86 (s, 6H); MS (EI) for C₃₂H₄₂N₄O₄: 547 (MH⁺).

1-(6,6-dimethyl-4-{7-[4-(methyloxy)phenyl]-2,3-dihydro-1,4-benzoxazepin-4(5H)-yl}-5,6,7,8-tetrahydroquinazolin-2-yl)-N,N-dimethylmethanamine. Prepared according to the method of example 3 by using 1-bromo-4-methoxybenzene in step 1 and 1-(4-chloro-6,6-dimethyl-5,6,7,8-tetrahydroquinazolin-2-yl)-N,N-dimethylmethanamine (reagent preparation 17) in step 3. ¹H NMR (400 MHz, (DMSO-d₆): 7.61-7.54 (m, 3H), 7.43-7.38 (m, 1H), 7.04-6.95 (m, 3H), 4.60 (s, 2H), 4.27 (m, 2H), 3.85 (m, 2H), 3.79 (s, 3H), 3.36 (m, 2H), 2.68 (t, 2H), 2.44 (s, 2H), 2.13 (s, 6H), 1.59 (t, 2H), 0.85 (s, 6H); MS (EI) for C₂₉H₃₆N₄O₂: 473 (MH⁺).

1-(4-{7-[3-chloro-4-(methyloxy)phenyl]-2,3-dihydro-1,4-benzoxazepin-4(5H)-yl}-6,6-dimethyl-5,6,7,8-tetrahydroquinazolin-2-yl)-N,N-dimethylmethanamine. Prepared according to the method of example 3 by using 4-bromo-2-chloro-1-methoxybenzene in step 1 and 1-(4-chloro-6,6-dimethyl-5,6,7,8-tetrahydroquinazolin-2-yl)-N,N-dimethylmethanamine (reagent preparation 17) in step 3. ¹H NMR (400 MHz, (DMSO-d₆): 7.70 (d, 1H), 7.65 (d, 1H), 7.62-7.57 (m, 1H), 7.48-7.43 (m, 1H), 7.22 (d, 1H), 6.98 (d, 1H), 4.60 (s, 2H), 4.28 (m, 2H), 3.89 (s, 3H), 3.85 (m, 2H), 3.36 (m, 2H), 2.69 (t, 2H), 2.44 (s, 2H), 2.13 (s, 6H), 1.59 (t, 2H), 0.86 (s, 6H); MS (EI) for C₂₉H₃₅ClN₄O₂: 507 (MH⁺).

### Example 4

### 4-(6,6-dimethyl-5,6,7,8-tetrahydroquinazolin-4-yl)-7-(2-propyl-3H-imidazo[4,5-b]pyridin-6-yl)-2,3,4,5-tetrahydro-1,4-benzoxazepine

STEP 1: A mixture of 2-amino-5-bromo-3-nitropyridine (0.31 g, 1.4 mmol [4-(6,6-dimethyl-5,6,7,8-tetrahydroquinazolin-4-yl)-2,3,4,5-tetrahydro-1,4-benzoxazepin-7-yl]boronic acid (0.50 g, 1.4 mmol), [1,1′-Bis(diphenylphosphino)ferrocene]dichloropalladium(II) (103 mg, 0.1 mmol), diisopropylethylamine (0.94 g, 7.1 mmol), 1,4-dioxane (20 mL), and water (2 mL) was stirred at 90° C. for 4 hours. The reaction mixture was cooled, diluted with ethyl acetate (100 mL) then filtered through celite. The filtrate was washed with 50 mL portion each of 5% aqueous citric acid, water, then brine solution, dried over sodium sulfate, filtered and concentrated to give a brown solid residue. It was washed with 35% ethyl acetate-hexane to give 5-[4-(6,6-dimethyl-5,6,7,8-tetrahydroquinazolin-4-yl)-2,3,4,5-tetrahydro-1,4-benzoxazepin-7-yl]-3-nitropyridin-2-amine (0.63 g, 97% yield). MS (EI) for C₂₄H₂₆N₆O₃: 447 (MH⁺).

STEP 2: A mixture of 5-[4-(6,6-dimethyl-5,6,7,8-tetrahydroquinazolin-4-yl)-2,3,4,5-tetrahydro-1,4-benzoxazepin-7-yl]-3-nitropyridin-2-amine (0.63 g, 1.4 mmol), 10% palladium on charcoal (0.50 g) in 30 mL of methanol was shaken in a Parr hydrogenation at 38 psi for 18 hours. The reaction mixture was filtered through a pad of celite and concentrated to give to give 5-[4-(6,6-dimethyl-5,6,7,8-tetrahydroquinazolin-4-yl)-2,3,4,5-tetrahydro-1,4-benzoxazepin-7-yl]pyridine-2,3-diamine (0.58 g, 99% yield). MS (EI) for C₂₄H₂₈N₆O: 417 (MH⁺).

STEP 3: To a solution of 5-[4-(6,6-dimethyl-5,6,7,8-tetrahydroquinazolin-4-yl)-2,3,4,5-tetrahydro-1,4-benzoxazepin-7-yl]pyridine-2,3-diamine (32 mg, 0.077 mmol) and triethylamine (11 uL, 0.077 mmol) in tetrahydrofuran (1 mL) was add butyryl chloride (7.0 uL, 0.077 mmol), and the resulting mixture was stirred at room temperature for one hour. The reaction mixture was concentrated to give N-{2-amino-5-[4-(6,6-dimethyl-5,6,7,8-tetrahydroquinazolin-4-yl)-2,3,4,5-tetrahydro-1,4-benzoxazepin-7-yl]pyridin-3-yl}butanamide crude product (50 mg). MS (EI) for C₂₈H₃₄N₆O₂: 487 (MH⁺).

STEP 4: N-{2-amino-5-[4-(6,6-dimethyl-5,6,7,8-tetrahydroquinazolin-4-yl)-2,3,4,5-tetrahydro-1,4-benzoxazepin-7-yl]pyridin-3-yl}butanamide (50 mg) was dissolved in acetic acid (0.2 mL) and heated in a microwave oven at 150 watts, 120° C. for 30 minutes. The reaction mixture was concentrated, dissolved in methanol (2 mL) and purified by preparative reverse phase HPLC (0.1% aqueous ammonium acetate-acetonitrile) to give 4-(6,6-dimethyl-5,6,7,8-tetrahydroquinazolin-4-yl)-7-(2-propyl-3H-imidazo[4,5-b]pyridin-6-yl)-2,3,4,5-tetrahydro-1,4-benzoxazepine (12 mg, 32% yield), ¹H NMR (400 MHz, Methanol-d₄): 8.51 (br, 1H), 8.37 (s, 1H), 8.06 (s, 1H), 7.61 (s, 1H), 7.51 (d, 1H), 7.10 (d, 1H), 4.74 (s, 2H), 4.36 (m, 2H), 3.96 (m, 2H), 2.94 (t, 2H), 2.77 (t, 2H), 2.48 (s, 2H), 1.89 (m, 2H), 1.66 (t, 2H), 1.01 (t, 3H), 0.81 (s, 6H); MS (EI) for C₂₈H₃₂N₆O: 469 (MH⁺).

Using analogous synthetic techniques and substituting with alternative starting reagents in steps 3 or step 4 the following compounds of the invention were prepared. Alternative starting materials were obtained commercially unless otherwise indicated.

4-(6,6-dimethyl-5,6,7,8-tetrahydroquinazolin-4-yl)-7-(2-ethyl-3H-imidazo[4,5-b]pyridin-6-yl)-2,3,4,5-tetrahydro-1,4-benzoxazepine. Prepared according to the method of example 4 by omitting step 3, and using trimethyl orthopropionate and 5-[4-(6,6-dimethyl-5,6,7,8-tetrahydroquinazolin-4-yl)-2,3,4,5-tetrahydro-1,4-benzoxazepin-7-yl]pyridine-2,3-diamine in step 4. ¹H NMR (400 MHz, Methanol-d₄): 8.77 (br, 1H), 8.34 (s, 1H), 8.06 (s, 1H), 7.59 (s, 1H), 7.50 (d, 1H), 7.09 (d, 1H), 4.74 (s, 2H), 4.38 (m, 2H), 3.94 (m, 2H), 2.97 (q, 2H), 2.77 (t, 2H), 2.47 (s, 2H), 1.67 (t, 2H), 1.26 (t, 3H), 0.90 (s, 6H); MS (EI) for C₂₇H₃₀N₆O: 455 (MH⁺).

7-(2-cyclopropyl-3H-imidazo[4,5-b]pyridin-6-yl)-4-(6,6-dimethyl-5,6,7,8-tetrahydroquinazolin-4-yl)-2,3,4,5-tetrahydro-1,4-benzoxazepine. Prepared according to the method of example 4 by using cyclopropylcarbonyl chloride in step 3. ¹H NMR (400 MHz, Methanol-d₄): 8.41 (br, 1H), 8.25 (s, 1H), 7.88 (s, 1H), 7.47 (s, 1H), 7.41 (d, 1H), 7.01 (d, 1H), 4.64 (s, 2H), 4.27 (m, 2H), 3.86 (m, 2H), 2.70 (t, 2H), 2.39 (s, 2H), 2.08 (m, 1H), 1.56 (t, 2H), 1.11 (m, 4H), 0.79 (s, 6H); MS (EI) for C₂₈H₃₀N₆O: 467 (MH⁺).

7-[2-(difluoromethyl)-3H-imidazo[4,5-b]pyridin-6-yl]-4-(6,6-dimethyl-5,6,7,8-tetrahydroquinazolin-4-yl)-2,3,4,5-tetrahydro-1,4-benzoxazepine. Prepared according to the method of example 4 by omitting step 3, and using difluoroacetic acid and 5-[4-(6,6-dimethyl-5,6,7,8-tetrahydroquinazolin-4-yl)-2,3,4,5-tetrahydro-1,4-benzoxazepin-7-yl]pyridine-2,3-diamine in step 4. ¹H NMR (400 MHz, Methanol-d₄): 8.70 (br, 1H), 8.34 (s, 1H), 8.23 (s, 1H), 7.64 (s, 1H), 7.55 (d, 1H), 7.12 (d, 1H), 7.05 (t, 1H), 4.63 (s, 2H), 4.37 (m, 2H), 3.95 (m, 2H), 2.79 (t, 2H), 2.49 (s, 2H), 1.66 (t, 2H), 0.88 (s, 6H); MS (EI) for C₂₆H₂₆F₂N₆O: 477 (MH⁺).

4-(6,6-dimethyl-5,6,7,8-tetrahydroquinazolin-4-yl)-7-[2-(fluoromethyl)-3H-imidazo[4,5-b]pyridin-6-yl]-2,3,4,5-tetrahydro-1,4-benzoxazepine hydrochloride. Prepared according to the method of example 4 by omitting step 3, and using fluoroacetic acid and 5-[4-(6,6-dimethyl-5,6,7,8-tetrahydroquinazolin-4-yl)-2,3,4,5-tetrahydro-1,4-benzoxazepin-7-yl]pyridine-2,3-diamine in step 4. ¹H NMR (400 MHz, Methanol-4₄): 8.66 (br, 1H), 8.52 (s, 1H), 8.21 (s, 1H), 7.74 (s, 1H), 7.56 (d, 1H), 7.09 (d, 1H), 5.69 (d, 1H), 5.13 (s, 2H), 4.47 (m, 2H), 4.32 (m, 2H), 2.84 (t, 2H), 2.61 (s, 2H), 1.69 (t, 2H), 0.94 (s, 6H); MS (EI) for C₂₆H₂₇FN₆O: 459 (MH⁺).

7-[2-(difluoromethyl)-1H-benzimidazol-5-yl]-4-(6,6-dimethyl-5,6,7,8-tetrahydroquinazolin-4-yl)-2,3,4,5-tetrahydro-1,4-benzoxazepine. Prepared according to the method of example 4 by omitting step 3, and using difluoroacetic acid and 5-[4-(6,6-dimethyl-5,6,7,8-tetrahydroquinazolin-4-yl)-2,3,4,5-tetrahydro-1,4-benzoxazepin-7-yl]pyridine-2,3-diamine in step 4. ¹H NMR (400 MHz, DMSO-d₆): 8.38 (s, 1H), 7.83 (br, 1H), 7.74 to 7.67 (m, 2H), 7.61 (d, 1H), 7.54 (d, 1H), 7.31 (t, 1H), 7.05 (d, 1H), 4.64 (s, 2H), 4.33 (m, 2H), 3.80 (m, 2H), 2.70 (t, 2H), 2.47 (s, 2H), 1.60 (t, 2H), 0.83 (s, 6H); MS (EI) for C₂₇H₂₇F₂N₅O: 476 (MH⁺).

### Example 5

### 4-(6,6-dimethyl-5,6,7,8-tetrahydroquinazolin-4-yl)-7-(2-methyl-1H-imidazo[4,5-b]pyridin-6-yl)-2,3,4,5-tetrahydro-1,4-benzoxazepine

STEP 1: A solution of 6-bromo-2-methyl-1-({[2-(trimethylsilyl)ethyl]oxy}methyl)-1H-imidazo[4,5-b]pyridine (reagent preparation 35) (0.43 g, 1.30 mmol), [4-(6,6-dimethyl-5,6,7,8-tetrahydroquinazolin-4-yl)-2,3,4,5-tetrahydro-1,4-benzoxazepin-7-yl]boronic acid (reagent preparation 23) (0.45 g, 1.30 mmol) and N,N-diisopropylethylamine (1.10 mL, 6.50 mmol) in N,N-dimethylformamide (4 mL), and water (1 mL) was degassed by bubbling nitrogen gas for five minutes followed by the addition of [1,1′-bis(diphenylphosphino)ferrocene]dichloropalladium(II) (52 mg, 0.065 mmol), then stirred at 95° C. for 16 hours. The reaction mixture was cooled to room temperature, diluted with ethyl acetate (100 mL), and filtered through a pad of Celite. The filtrate was washed with 1M aqueous lithium chloride (50 mL) and brine, dried over sodium sulfate, filtered and concentrated. Column chromatography on silica (gradient 1-2% 7N ammonia in methanol in chloroform) gave 4-(6,6-dimethyl-5,6,7,8-tetahydroquinazolin-4-yl)-7-[2-methyl-1-({[2-(trimethylsilyl)ethyl]oxy}methyl)-1H-imidazo[4,5-b]pyridin-6-yl]-2,3,4,5-tetrahydro-1,4-benzoxazepine (0.54 g, 73% yield). MS (EI) for C₃₂H₄₂N₆O₂Si: 571 (MH⁺).

STEP 2: A solution of 4-(6,6-dimethyl-5,6,7,8-tetrahydroquinazolin-4-yl)-7-[2-methyl-1-({[2-(trimethylsilyl)ethyl]oxy}methyl)-1H-imidazo[4,5-b]pyridin-6-yl]-2,3,4,5-tetrahydro-1,4-benzoxazepine (0.54 g, 0.95 mmol) in a mixture of methanol (30 mL) and concentrated hydrochloric acid (1 mL) was stirred at reflux for 16 hours. On cooling to room temperature the solution was concentrated and the pH was adjusted to ˜9 by the addition of 50% aqueous sodium hydroxide and diluted with ethyl acetate (100 mL). The organic layer was washed with 2M aqueous sodium hydroxide (20 mL) and brine, dried over anhydrous sodium sulfate, filtered and concentrated. The precipitating white solid was collected by filtration and washed with hexane to provide 4-(6,6-dimethyl-5,6,7,8-tetrahydroquinazolin-4-yl)-7-(2-methyl-1H-imidazo[4,5-b]pyridin-6-yl)-2,3,4,5-tetrahydro-1,4-benzoxazepine (0.28 g, 67%). ¹H NMR (400 MHz, d₆-DMSO): 8.53 (d, 1H), 8.38 (s, 1H), 8.03 (d, 1H), 7.70 (d, 1H), 7.54 (dd, 1H), 7.05 (d, 1H), 4.63 (s, 2H), 4.33 (m, 2H), 3.85 (m, 2H), 2.72 (m, 2H), 2.58 (s, 3H), 2.47 (s, 2H), 1.60 (m, 2H), 0.86 (s, 6H); MS (EI) for C₂₆H₂₈N₆O: 441 (MH⁺).

Using analogous synthetic techniques and substituting with alternative starting reagents in steps 1 and conducting protecting group removal in step 2 as required according to literature techniques appropriate for a given protecting group (see for example: Greene and Wuts, Protective Groups in Organic Synthetic, Wiley-Interscience) the following compounds of the invention were prepared. Alternative starting materials were obtained commercially unless otherwise indicated.

6-[4-(6,6-dimethyl-5,6,7,8-tetrahydroquinazolin-4-yl)-2,3,4,5-tetrahydro-1,4-benzoxazepin-7-yl][1,3]thiazolo[5,4-b]pyridin-2-amine. Prepared according to the method of example 5 by using N-(6-bromo[1,3]thiazolo[5,4-b]pyridin-2-yl)acetamide (Journal of Heterocyclic Chemistry (2003), 40, 261-268) and [4-(6,6-dimethyl-5,6,7,8-tetrahydroquinazolin-4-yl)-2,3,4,5-tetrahydro-1,4-benzoxazepin-7-yl]boronic acid (reagent preparation 23) in step 1. ¹H NMR (400 MHz, d₆-DMSO): 8.38 (m, 2H), 7.88 (brs, 2H), 7.83 (d, 1H), 7.72 (d, 1H), 7.76 (dd, 1H), 7.05 (d, 1H), 4.63 (s, 2H), 4.33 (m, 2H), 3.83 (m, 2H), 2.72 (m, 2H), 2.46 (s, 2H), 1.61 (m, 2H), 0.83 (s, 6H); MS (EI) for C₂₅H₂₆N₆OS: 459 (MH⁺).

4-(6,6-dimethyl-5,6,7,8-tetrahydroquinazolin-4-yl)-7-(1H-pyrazolo[3,4-b]pyridin-5-yl)-2,3,4,5-tetrahydro-1,4-benzoxazepine. Prepared according to the method of example 5 by using 5-bromo-1-(tetrahydro-2H-pyran-2-yl)-1H-pyrazolo[3,4-b]pyridine (reagent preparation 32) and [4-(6,6-dimethyl-5,6,7,8-tetrahydroquinazolin-4-yl)-2,3,4,5-tetrahydro-1,4-benzoxazepin-7-yl]boronic acid (reagent preparation 23) in step 1. ¹H NMR (400 MHz, d₆-DMSO): 13.71 (s, 1H), 8.82 (d, 1H), 8.43 (d, 1H), 8.38 (s, 1H), 8.18 (d, 1H), 7.74 (d, 1H), 7.58 (dd, 1H), 7.07 (d, 1H), 4.66 (s, 2H), 4.34 (m, 2H), 3.85 (m, 2H), 2.71 (m, 2H), 2.45 (s, 2H), 1.59 (m, 2H), 0.84 (s, 6H); MS (EI) for C₂₅H₂₆N₆O: 427 (MH⁺).

4-(6,6-dimethyl-5,6,7,8-tetrahydroquinazolin-4-yl)-7-(1H-imidazo[4,5-b]pyridin-6-yl)-2,3,4,5-tetrahydro-1,4-benzoxazepine. Prepared according to the method of example 5 by using 6-bromo-1-(triphenylmethyl)-1H-imidazo[4,5-b]pyridine (reagent preparation 15) and [4-(6,6-dimethyl-5,6,7,8-tetrahydroquinazolin-4-yl)-2,3,4,5-tetrahydro-1,4-benzoxazepin-7-yl]boronic acid (reagent preparation 23) in step 1. ¹H NMR (400 MHz, d₆-DMSO): 8.64 (s, 1H), 8.48 (s, 1H), 8.39 (s, 1H), 8.22 (brs, 1H), 7.75 (d, 1H), 7.58 (dd, 1H), 7.06 (d, 1H), 4.67 (s, 2H), 4.35 (m, 2H), 3.85 (m, 2H), 2.72 (m, 2H), 2.46 (s, 2H), 1.61 (m, 2H), 0.85 (s, 6H); MS (EI) for C₂₅H₂₆N₆O: 427 (MH⁺).

3-{[(3R)-3-aminopyrrolidin-1-yl]sulfonyl}-5-[4-(6,6-dimethyl-5,6-dihydroquinazolin-4-yl)-2,3,4,5-tetrahydro-1,4-benzoxazepin-7-yl]pyridin-2-amine. Prepared according to the method of example 5 by using [4-(6,6-dimethyl-5,6-dihydroquinazolin-4-yl)-2,3,4,5-tetrahydro-1,4-benzoxazepin-7-yl]boronic acid (reagent preparation 23) and 1,1-dimethylethyl{(3R)-1-[(2-amino-5-bromopyridin-3-yl)sulfonyl]pyrrolidin-3-yl}carbamate (reagent preparation 25) in step 1. ¹H NMR (400 MHz, d₆-DMSO): 8.57 (d, 1H), 8.41 (s, 1H), 8.02 (d, 1H), 7.58 (d, 1H), 7.47 (dd, 1H), 7.02 (d, 1H), 6.75 (brs, 2H), 6.26 (dd, 2H), 4.63 (s, 2H), 4.33 (m, 2H), 3.84 (m, 2H), 3.37 (m, 2H), 3.27 (m, 2H), 2.89 (m, 1H), 2.71 (s, 2H), 1.86 (m, 3H), 1.53 (m, 1H), 0.95 (s, 6H); MS (EI) for C₂₈H₃₃N₇O₃S: 548 (MH⁺).

3-{[(3R)-3-aminopyrrolidin-1-yl]sulfonyl}-5-{4-[(7S)-7-ethyl-5,6,7,8-tetrahydroquinazolin-4-yl]-2,3,4,5-tetrahydro-1,4-benzoxazepin-7-yl}pyridin-2-amine. Prepared according to the method of example 5 by using {4-[(7S)-7-ethyl-5,6,7,8-tetrahydroquinazolin-4-yl]-2,3,4,5-tetrahydro-1,4-benzoxazepin-7-yl}boronic acid (reagent preparation 23) and 1,1-dimethylethyl{(3R)-1-[(2-amino-5-bromopyridin-3-yl)sulfonyl]pyrrolidin-3-yl}carbamate (reagent preparation 25) in step 1. ¹H NMR (400 MHz, d₆-DMSO): 8.53 (d, 1H), 8.33 (s, 1H), 7.99 (d, 1H), 7.57 (d, 1H), 7.44 (dd, 1H), 7.01 (d, 1H), 6.75 (brs, 2H), 4.70 (dd, 2H), 4.36 (m, 1H), 4.24 (m, 1H), 3.86 (m, 2H), 3.36 (m, 2H), 3.26 (m, 2H), 2.87, 2.83 (m, dd, 3H), 2.27 (dd, 1H), 1.89 (m, 3H), 1.68 (m, 1H), 1.52 (m, 1H), 1.37 (m, 2H), 1.11 (m, 1H), 0.94 (t, 3H); MS (EI) for C₂₈H₃₅N₇O₃S: 550 (MH⁺).

2-amino-N-(2-amino-2-methylpropyl)-5-{4-[(7S)-7-ethyl-5,6,7,8-tetrahydroquinazolin-4-yl]-2,3,4,5-tetrahydro-1,4-benzoxazepin-7-yl}pyridine-3-sulfonamide. Prepared according to the method of example 5 by using {4-[(7S)-7-ethyl-5,6,7,8-tetrahydroquinazolin-4-yl]-2,3,4,5-tetrahydro-1,4-benzoxazepin-7-yl}boronic acid (reagent preparation 23) and 2-amino-N-(2-amino-2-methylpropyl)-5-bromopyridine-3-sulfonamide (reagent preparation 25) in step 1. ¹H NMR (400 MHz, d₆-DMSO): 8.48 (d, 1H), 8.33 (s, 1H), 8.01 (d, 1H), 7.54 (d, 1H), 7.42 (dd, 1H), 7.01 (d, 1H), 7.72 (brs, 2H), 4.69 (dd, 2H), 4.37 (m, 1H), 4.23 (m, 1H), 3.85 (m, 2H), 2.93 (m, 2H), 2.60 (s, 2H), 2.27 (dd, 1H), 1.87 (m, 1H), 1.70 (m, 1H), 1.35 (m, 2H), 1.10 (m, 1H), 0.93 (t, 3H), 0.92 (s, 6H); MS (EI) for C₂₈H₃₇N₇O₃S: 552 (MH⁺).

7-(2-methyl-1H-imidazo[4,5-b]pyridin-6-yl)-4-[7-(methyloxy)quinazolin-4-yl]-2,3,4,5-tetrahydro-1,4-benzoxazepine. Prepared as hydrochloride, according to the method of example 5 by using {4-[7-(methyloxy)quinazolin-4-yl]-2,3,4,5-tetrahydro-1,4-benzoxazepin-7-yl}boronic acid (reagent preparation 23) and 6-bromo-2-methyl-1-({[2-(trimethylsilyl)ethyl]oxy}methyl)-1H-imidazo[4,5-b]pyridine (reagent preparation 35) in step 1. ¹H NMR (400 MHz, d₆-DMSO): 8.84 (d, 1H), 8.78 (s, 1H), 8.39 (d, 1H), 8.24 (brd, 1H), 7.95 (d, 1H), 7.68 (dd, 1H), 7.35 (d, 1H), 7.31 (s, 1H), 7.04 (d, 1H), 5.46 (s, 2H), 4.68 (m, 2H), 4.50 (m, 2H), 3.96 (s, 3H), 2.78 (s, 3H); MS (EI) for C₂₅H₂₂N₆O₂: 439 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-[2-(methyloxy)ethyl]pyridine-3-sulfonamide. Prepared according to the method of example 5 by using 2-amino-5-bromo-N-(2-methoxyethyl)pyridine-3-sulfonamide (reagent preparation 25) in step 1. ¹H NMR (400 MHz, DMSO-d6) δ 8.51 (d, 1H), 8.37 (s, 1H), 8.06 (d, 1H), 7.96 (br s, 1H), 7.59 (d, 1H), 7.45 (dd, 1H), 7.02 (d, 1H), 6.69 (br s, 2H), 4.62 (s, 2H), 4.35-4.28 (m, 2H), 3.86-3.80 (m, 2H), 3.29 (t, 2H), 3.15 (s, 3H), 2.94 (t, 2H), 2.71 (t, 2H), 2.44 (s, 2H), 1.60 (t, 2H), 0.84 (s, 6H); MS (EI) for C₂₇H₃₄H₆O₄S: 539 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-(2,2,2-trifluoroethyl)pyridine-3-sulfonamide. Prepared according to the method of example 5 by using 2-amino-5-bromo-N-(2,2,2-trifluoroethyl)pyridine-3-sulfonamide (reagent preparation 25) in step 1. ¹H NMR (400 MHz, DMSO-d6) δ 8.43 (d, 1H), 8.36 (s, 1H), 8.04 (d, 1H), 7.56 (s, 1H), 7.46-7.39 (m, 2H), 7.02 (d, 1H), 6.56 (br s, 2H), 5.16-5.07 (m, 2H), 4.62 (s, 2H), 4.35-4.26 (m, 2H), 3.86-3.80 (m, 2H), 2.71 (t, 2H), 2.45 (s, 2H), 1.60 (t, 2H), 0.85 (s, 6H); MS (EI) for C₂₆H₂₉F₃N₆O₃S: 563 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-(2-hydroxyethyl)-N-methylpyridine-3-sulfonamide. Prepared according to the method of example 5 by using 2-amino-5-bromo-N-(2-hydroxyethyl)-N-methylpyridine-3-sulfonamide (reagent preparation 25) in step 1. ¹H NMR (400 MHz, DMSO-d6) δ 8.54 (d, 1H), 8.37 (s, 1H), 8.00 (d, 1H), 7.60 (d, 1H), 7.47 (dd, 1H), 7.02 (d, 1H), 6.79 (br s, 2H), 4.87 (t, 1H), 4.62 (s, 2H), 4.35-4.28 (m, 2H), 3.89-3.77 (m, 2H), 3.54 (q, 2H), 3.19 (t, 2H), 2.79 (s, 3H), 2.71 (s, 2H), 2.45 (s, 2H), 1.61 (d, 2H), 0.85 (s, 6H); MS (EI) for C₂₇H₃₄N₆O₄S: 539 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-(2-hydroxypropyl)pyridine-3-sulfonamide. Prepared according to the method of example 5 by using 2-amino-5-bromo-N-(2-hydroxypropyl)pyridine-3-sulfonamide (reagent preparation 25) in step 1. ¹H NMR (400 MHz, DMSO-d6) δ 8.50 (d, 1H), 8.37 (s, 1H), 8.05 (d, 1H), 7.58 (d, 1H), 7.45 (dd, 1H), 7.02 (d, 1H), 6.71 (br s, 2H), 4.62 (s, 2H), 4.35-4.27 (m, 2H), 3.87-3.80 (m, 2H), 3.63-3.55 (m, 1H), 2.75-2.64 (m, 4H), 2.44 (s, 2H), 1.60 (t, 2H), 0.98 (d, 3H), 0.84 (d, 6H); MS (EI) for C₂₇H₃₄N₆O₄S: 539 (MH⁺).

2-amino-N-azetidin-3-yl-5-[4-(6,6-dimethyl-5,6,7,8-tetrahydroquinazolin-4-yl)-2,3,4,5-tetrahydro-1,4-benzoxazepin-7-yl]pyridine-3-sulfonamide. Prepared as a diacetate salt according to the method of example 5 by using tert-butyl 3-(2-amino-5-bromopyridine-3-sulfonamido)azetidine-1-carboxylate (reagent preparation 25) in step 1 followed by Boc deprotection. ¹H NMR (400 MHz, DMSO-d6) δ 8.51 (d, 1H), 8.37 (s, 1H), 8.03 (d, 1H), 7.59 (d, 1H), 7.45 (dd, 1H), 7.03 (d, 1H), 6.70 (br s, 2H), 4.62 (s, 2H), 4.35-4.27 (m, 2H), 4.00-3.90 (m, 1H), 3.86-3.79 (m, 2H), 3.25 (d, 4H), 2.71 (t, 2H), 2.44 (s, 2H), 1.88 (s, 6H), 1.60 (t, 2H), 0.84 (s, 6H); MS (EI) for C₂₇H₃₃N₇O₃S: 536 (MH⁺).

2-amino-N-(2,3-dihydroxypropyl)-5-[4-(6,6-dimethyl-5,6,7,8-tetrahydroquinazolin-4-yl)-2,3,4,5-tetrahydro-1,4-benzoxazepin-7-yl]pyridine-3-sulfonamide. Prepared according to the method of example 5 by using 2-amino-5-bromo-N-(2,3-dihydroxypropyl)pyridine-3-sulfonamide (reagent preparation 25) in step 1. ¹H NMR (400 MHz, DMSO-d6) δ 8.50 (d, 1H), 8.37 (s, 1H), 8.05 (d, 1H), 7.58 (d, 1H), 7.44 (dd, 1H), 7.02 (d, 1H), 6.72 (br s, 2H), 4.85 (br s, 1H), 4.66-4.51 (m, 3H), 4.36-4.26 (m, 2H), 3.87-3.78 (m, 2H), 3.50-3.43 (m, 1H), 3.29-3.19 (m, 2H), 2.90 (dd, 1H), 2.71 (t, 2H), 2.62 (dd, 1H), 2.44 (s, 2H), 1.60 (t, 2H), 0.85 (s, 6H); MS (EI) for C₂₇H₃₄N₆O₅S: 555 (MH⁺).

1-({2-amino-5-[4-(6,6-dimethyl-5,6,7,8-tetrahydroquinazolin-4-yl)-2,3,4,5-tetrahydro-1,4-benzoxazepin-7-yl]pyridin-3-yl}sulfonyl)piperidin-3-ol. Prepared according to the method of example 5 by using 1-(2-amino-5-bromopyridin-3-ylsulfonyl)piperidin-3-ol (reagent preparation 25) in step 1. ¹H NMR (400 MHz, DMSO-d6) δ 8.56 (d, 1H), 8.37 (s, 1H), 7.94 (d, 1H), 7.59 (d, 1H), 7.47 (dd, 1H), 7.03 (d, 1H), 6.78 (br s, 2H), 5.00 (d, 1H), 4.62 (s, 2H), 4.36-4.27 (m, 2H), 3.88-3.78 (m, 2H), 3.60-3.43 (m, 2H), 3.42-3.29 (m, 1H), 2.71 (t, 2H), 2.60 (t, 1H), 2.47-2.42 (m, 3H), 1.79-1.67 (m, 2H), 1.60 (t, 2H), 1.50-1.36 (m, 1H), 1.26-1.12 (m, 1H), 0.84 (s, 6H); MS (EI) for C₂₉H₃₆N₆O₄S: 565 (MH⁺).

2-amino-N-(3-amino-2,2-dimethylpropyl)-5-[4-(6,6-dimethyl-5,6,7,8-tetrahydroquinazolin-4-yl)-2,3,4,5-tetrahydro-1,4-benzoxazepin-7-yl]pyridine-3-sulfonamide. Prepared as an acetate salt according to the method of example 5 by using 2-amino-N-(3-amino-2,2-dimethylpropyl)-5-bromopyridine-3-sulfonamide (reagent preparation 25) in step 1. ¹H NMR (400 MHz, DMSO-d6) δ 8.50 (d, 1H), 8.37 (s, 1H), 8.05 (d, 1H), 7.58 (d, 1H), 7.45 (dd, 1H), 7.02 (d, 1H), 6.71 (br s, 2H), 4.62 (s, 2H), 4.35-4.26 (m, 2H), 3.87-3.78 (m, 2H), 2.71 (t, 2H), 2.58 (s, 2H), 2.44 (s, 2H), 2.34 (s, 2H), 1.88 (s, 3H), 1.59 (t, 2H), 0.84 (s, 6H), 0.75 (s, 6H); MS (EI) for C₂₉H₃₉N₇O₃S: 566 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-(3-hydroxy-2,2-dimethylpropyl)pyridine-3-sulfonamide. Prepared according to the method of example 5 by using 2-amino-5-bromo-N-(3-hydroxy-2,2-dimethylpropyl)pyridine-3-sulfonamide (reagent preparation 25) in step 1. ¹H NMR (400 MHz, DMSO-d6) δ 8.50 (d, 1H), 8.37 (s, 1H), 8.04 (d, 1H), 7.62-7.55 (m, 2H), 7.45 (dd, 1H), 7.02 (d, 1H), 6.72 (br s, 2H), 4.62 (s, 2H), 4.50 (t, 1H), 4.34-4.28 (m, 2H), 3.86-3.79 (m, 2H), 3.08 (d, 2H), 2.71 (t, 2H), 2.58 (d, 2H), 2.44 (s, 2H), 1.60 (t, 2H), 0.84 (s, 6H), 0.75 (s, 6H); MS (EI) for C₂₉H₃₈N₆O₄S: 567 (MH⁺).

N-{5-[4-(6,6-dimethyl-5,6,7,8-tetrahydroquinazolin-4-yl)-2,3,4,5-tetrahydro-1,4-benzoxazepin-7-yl]pyridin-3-yl}methanesulfonamide. Prepared according to the method of example 5 by using N-(5-bromopyridin-3-yl)methanesulfonamide (reagent preparation 26) in step 1. ¹H NMR (400 MHz, DMSO-d6) δ 10.11 (s, 1H), 8.61 (d, 1H), 8.42-8.33 (m, 2H), 7.79 (t, 1H), 7.67 (d, 1H), 7.51 (dd, 1H), 7.08 (d, 1H), 4.67 (s, 2H), 4.39-4.32 (m, 2H), 3.88-3.81 (m, 2H), 3.11 (s, 3H), 2.71 (t, 2H), 2.43 (s, 2H), 1.59 (t, 2H), 0.84 (s, 6H); MS (EI) for C₂₅H₂₉N₅O₃S: 480 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-(2-hydroxy-1,1-dimethylethyl)pyridine-3-sulfonamide. Prepared as an acetate salt according to the method of example 5 by using 2-amino-5-bromo-N-(1-hydroxy-2-methylpropan-2-yl)pyridine-3-sulfonamide (reagent preparation 25) in step 1. ¹H NMR (400 MHz, DMSO-d6) δ 8.48 (d, 1H), 8.37 (s, 1H), 8.09 (d, 1H), 7.56 (d, 1H), 7.43 (dd, 1H), 7.02 (d, 1H), 6.69 (br s, 2H), 4.61 (s, 2H), 4.35-4.28 (m, 2H), 3.88-3.79 (m, 2H), 3.21 (s, 2H), 2.71 (t, 2H), 2.44 (s, 2H), 1.82 (s, 3H), 1.60 (t, 2H), 1.03 (s, 6H), 0.84 (s, 6H); MS (EI) for C₂₈H₃₆N₆O₄S: 553 (MH⁺).

2-chloro-N-{2-chloro-5-[4-(6,6-dimethyl-5,6,7,8-tetrahydroquinazolin-4-yl)-2,3,4,5-tetrahydro-1,4-benzoxazepin-7-yl]pyridin-3-yl}-6-methylbenzenesulfonamide Prepared according to the method of example 5 by using N-(5-bromo-2-chloropyridin-3-yl)-2-chloro-6-methylbenzenesulfonamide (reagent preparation 26) in step 1. ¹H NMR (400 MHz, DMSO-d6) δ 8.48 (br s, 1H), 8.39 (s, 1H), 7.94 (d, 1H), 7.62 (d, 1H), 7.51-7.41 (m, 3H), 7.34 (dd, 1H), 7.06 (d, 1H), 4.68 (s, 2H), 4.41-4.34 (m, 2H), 3.90-3.83 (m, 2H), 2.71 (t, 2H), 2.52 (s, 3H), 2.40 (s, 2H), 1.59 (t, 2H), 0.82 (s, 6H); MS (EI) for C₃₁H₃₁Cl₂N₅O₃S: 624, 626 (Cl isotopes, MH⁺).

N-{5-[4-(6,6-dimethyl-5,6,7,8-tetrahydroquinazolin-4-yl)-2,3,4,5-tetrahydro-1,4-benzoxazepin-7-yl]-2-fluoropyridin-3-yl}methanesulfonamide. Prepared according to the method of example 5 by using N-(5-bromo-2-fluoropyridin-3-yl)methanesulfonamide (reagent preparation 26) in step 1. ¹H NMR (400 MHz, DMSO-d6) δ 8.37 (s, 1H), 8.27 (s, 1H), 8.07 (d, 1H), 7.68 (s, 1H), 7.53 (d, 1H), 7.08 (d, 1H), 4.67 (s, 2H), 4.40-4.32 (m, 2H), 3.89-3.82 (m, 2H), 3.15 (s, 3H), 2.71 (t, 2H), 2.43 (s, 2H), 1.60 (t, 2H), 0.85 (s, 6H); MS (EI) for C₂₅H₂₈FN₅O₃S: 498 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-(piperidin-4-ylmethyl)pyridine-3-sulfonamide. Prepared as an acetate salt according to the method of example 5 by using tert-butyl 44(2-amino-5-bromopyridine-3-sulfonamido)methyl)piperidine-1-carboxylate (reagent preparation 25) in step 1 followed by Boc deprotection. ¹H NMR (400 MHz, DMSO-d6) δ 8.51 (d, 1H), 8.37 (s, 1H), 8.05 (d, 1H), 7.59 (d, 1H), 7.45 (dd, 1H), 7.02 (d, 1H), 6.68 (br s, 2H), 4.62 (s, 2H), 4.35-4.28 (m, 2H), 3.87-3.79 (m, 2H), 2.97-2.87 (m, 2H), 2.71 (t, 2H), 2.63 (d, 2H), 2.39 (dd, 4H), 1.84 (s, 3H), 1.64-1.51 (m, 4H), 1.51-1.35 (m, 1H), 1.04-0.90 (m, 2H), 0.84 (s, 6H); MS (EI) for C₃₀H₃₉N₇O₃S: 578 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-[(1-methylpiperidin-4-yl)methyl]pyridine-3-sulfonamide. Prepared as a diacetate salt according to the method of example 5 by using 2-amino-5-bromo-N-((1-methylpiperidin-4-yl)methyl)pyridine-3-sulfonamide (reagent preparation 25) in step 1. ¹H NMR (400 MHz, DMSO-d6) δ 8.51 (d, 1H), 8.37 (s, 1H), 8.04 (d, 1H), 7.58 (d, 1H), 7.45 (dd, 1H), 7.02 (d, 1H), 6.67 (br s, 2H), 4.62 (s, 2H), 4.36-4.28 (m, 2H), 3.87-3.79 (m, 2H), 2.76-2.60 (m, 6H), 2.44 (s, 2H), 2.08 (s, 3H), 1.89 (s, 6H), 1.75-1.66 (m, 2H), 1.63-1.52 (m, 4H), 1.32-1.20 (m, 1H), 1.10-0.95 (m, 2H), 0.84 (s, 6H); MS (S) for C₃₁H₄₁N₇O₃S: 592 (MH⁺).

2-amino-N-[(1-aminocyclopropyl)methyl]-5-[4-(6,6-dimethyl-5,6,7,8-tetrahydroquinazolin-4-yl)-2,3,4,5-tetrahydro-1,4-benzoxazepin-7-yl]pyridine-3-sulfonamide. Prepared as a diacetate salt according to the method of example 5 by using tert-butyl 1-((2-amino-5-bromopyridine-3-sulfonamido)methyl)cyclopropylcarbamate (reagent preparation 25) in step 1 followed by Boc deprotection. ¹H NMR (400 MHz, DMSO-d6) δ 8.50 (d, 1H), 8.37 (s, 1H), 8.05 (d, 1H), 7.58 (d, 1H), 7.45 (dd, 1H), 7.02 (d, 1H), 6.70 (br s, 2H), 4.62 (s, 2H), 4.34-4.27 (m, 2H), 3.86-3.79 (m, 2H), 2.77 (s, 2H), 2.70 (t, 2H), 2.44 (s, 2H), 1.89 (s, 6H), 1.59 (t, 2H), 0.84 (s, 6H), 0.32 (d, 1H); MS (EI) for C₂₈H₃₅N₇O₃S: 550 (MH⁺).

2-amino-N-(trans-4-aminocyclohexyl)-5-[4-(6,6-dimethyl-5,6,7,8-tetrahydroquinazolin-4-yl)-2,3,4,5-tetrahydro-1,4-benzoxazepin-7-yl]pyridine-3-sulfonamide. Prepared according to the method of example 5 by using tert-butyl trans-4-(2-amino-5-bromopyridine-3-sulfonamido)cyclohexylcarbamate (reagent preparation 25) in step 1 followed by Boc deprotection. ¹H NMR (400 MHz, DMSO-d6) δ 8.51 (d, 1H), 8.37 (s, 1H), 8.07 (d, 1H), 7.59 (d, 1H), 7.46 (dd, 1H), 7.03 (d, 1H), 6.66 (br s, 2H), 4.62 (s, 2H), 4.35-4.28 (m, 2H), 3.87-3.80 (m, 2H), 2.89-2.78 (m, 1H), 2.71 (t, 2H), 2.44 (s, 2H), 2.42-2.36 (m, 1H), 1.68-1.55 (m, 6H), 1.27-1.07 (m, 2H), 0.98-0.81 (m, 8H); MS (EI) for C₃₀H₃₉N₇O₃S: 578 (MH⁺).

2-amino-N-[(1-methylpiperidin-4-yl)methyl]-5-[4-(2,6,6-trimethyl-5,6,7,8-tetrahydroquinazolin-4-yl)-2,3,4,5-tetrahydro-1,4-benzoxazepin-7-yl]pyridine-3-sulfonamide. Prepared as a diacetate salt according to the method of example 5 by using [4-(2,6,6-trimethyl-5,6,7,8-tetrahydroquinazolin-4-yl)-2,3,4,5-tetrahydro-1,4-benzoxazepin-7-yl]boronic acid (reagent preparation 23) and 2-amino-5-bromo-N-((1-methylpiperidin-4-yl)methyl)pyridine-3-sulfonamide (reagent preparation 25) in step 1. ¹H NMR (400 MHz, DMSO-d6) δ 8.51 (d, 1H), 8.05 (d, 1H), 7.60 (d, 1H), 7.45 (dd, 1H), 7.03 (d, 1H), 6.67 (br s, 2H), 4.57 (s, 2H), 4.30-4.24 (m, 2H), 3.86-3.79 (m, 2H), 2.70-2.61 (m, 6H), 2.40 (s, 2H), 2.33 (s, 3H), 2.08 (s, 3H), 1.87 (s, 6H), 1.69 (t, 2H), 1.62-1.50 (m, 4H), 1.32-1.19 (m, 1H), 1.08-0.95 (m, 2H), 0.84 (s, 6H); MS (EI) for C₃₂H₄₃N₇O₃S: 606 (MH⁺).

2-amino-N-[(1-methylpiperidin-4-yl)methyl]-5-[4-(6,6,7-trimethyl-5,6-dihydroquinazolin-4-yl)-2,3,4,5-tetrahydro-1,4-benzoxazepin-7-yl]pyridine-3-sulfonamide. Prepared according to the method of example 5 by using [4-(6,6,7-trimethyl-5,6-dihydroquinazolin-4-yl)-2,3,4,5-tetrahydro-1,4-benzoxazepin-7-yl]boronic acid (reagent preparation 23) and 2-amino-5-bromo-N-((1-methylpiperidin-4-yl)methyl)pyridine-3-sulfonamide (reagent preparation 25) in step 1. ¹H NMR (400 MHz, DMSO-d6) δ 8.50 (d, 1H), 8.37 (s, 1H), 8.03 (d, 1H), 7.84 (t, 1H), 7.55 (d, 1H), 7.45 (dd, 1H), 7.02 (d, 1H), 6.67 (br s, 2H), 6.13 (d, 1H), 4.62 (s, 2H), 4.37-4.29 (m, 2H), 3.88-3.78 (m, 2H), 2.72-2.60 (m, 6H), 2.07 (s, 3H), 1.88 (s, 3H), 1.74-1.62 (m, 2H), 1.61-1.50 (m, 2H), 1.34-1.16 (m, 1H), 1.09-0.95 (m, 2H), 0.91 (s, 6H); MS (EI) for C₃₂H₄₁H₇O₃S: 604 (MH⁺).

2-amino-N-(2-aminopropyl)-5-[4-(6,6-dimethyl-5,6,7,8-tetrahydroquinazolin-4-yl)-2,3,4,5-tetrahydro-1,4-benzoxazepin-7-yl]pyridine-3-sulfonamide. Prepared according to the method of example 5 by using benzyl 1-(2-amino-5-bromopyridine-3-sulfonamido)propan-2-ylcarbamate (reagent preparation 25) in step 1 followed by Cbz deprotection. ¹H NMR (400 MHz, DMSO-d6) δ 8.49 (d, 1H), 8.37 (s, 1H), 8.04 (d, 1H), 7.58 (s, 1H), 7.45 (dd, 1H), 7.02 (d, 1H), 6.69 (br s, 2H), 4.62 (s, 2H), 4.35-4.27 (m, 2H), 3.87-3.79 (m, 2H), 3.01 (t, 2H), 2.71 (s, 2H), 2.60 (t, 1H), 2.45 (s, 2H), 1.78-1.70 (m, 3H), 1.60 (s, 2H), 0.85 (s, 6H); MS (EI) for C₂₇H₃₅N₇O₃S: 538 (MH⁺).

N-{2-chloro-5-[4-(6,6-dimethyl-5,6,7,8-tetrahydroquinazolin-4-yl)-2,3,4,5-tetrahydro-1,4-benzoxazepin-7-yl]pyridin-3-yl}acetamide. Prepared according to the method of example 5 by using N-(5-bromo-2-chloropyridin-3-yl)acetamide (reagent preparation 26) in step 1. ¹H NMR (400 MHz, DMSO-d6) δ 9.84 (s, 1H), 8.68 (s, 1H), 8.52 (d, 1H), 8.43 (d, 1H), 7.77 (d, 1H), 7.57 (dd, 1H), 7.04 (d, 1H), 5.08 (s, 2H), 4.54-4.45 (m, 2H), 4.23-4.13 (m, 2H), 2.77 (t, 2H), 2.54 (s, 2H), 2.17 (d, 3H), 1.58 (t, 2H), 0.86 (s, 6H); MS (EI) for C₂₆H₂₈ClN₅O₂: 478 (MH⁺).

methyl{2-chloro-5-[4-(6,6-dimethyl-5,6,7,8-tetrahydroquinazolin-4-yl)-2,3,4,5-tetrahydro-1,4-benzoxazepin-7-yl]pyridin-3-yl}carbamate. Prepared according to the method of example 5 by using methyl 5-bromo-2-chloropyridin-3-ylcarbamate (reagent preparation 26) in step 1. ¹H NMR (400 MHz, CD₃OD) δ 8.56 (d, 1H), 8.36-8.31 (m, 2H), 7.60 (d, 1H), 7.51 (dd, 1H), 7.09 (d, 1H), 4.73 (s, 2H), 4.40-4.32 (m, 2H), 4.00-3.92 (m, 2H), 3.82 (s, 3H), 2.80 (t, 2H), 2.48 (s, 2H), 1.69 (t, 2H), 0.90 (s, 6H); MS (EI) for C₂₆H₂₈ClN₅O₃: 494 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-ethylpyridine-3-sulfonamide. Prepared by the method of example 5 using 2-amino-5-bromo-N-ethylppidine-3-sulfonamide (reagent preparation 25) in step 1. ¹H NMR (400 MHz, DMSO-d6) δ 8.50 (d, 1H), 8.37 (s, 1H), 8.04 (d, 1H), 7.79 (br s, 1H), 7.61-7.48 (m, 2H), 7.45 (dd, 1H), 7.02 (d, 1H), 6.67 (br s, 2H), 4.62 (s, 2H), 4.36-4.27 (m, 2H), 3.88-3.77 (m, 2H), 2.80 (q, 2H), 2.70 (t, 2H), 2.44 (s, 2H), 1.60 (t, 2H), 0.97 (t, 3H), 0.84 (s, 6H); MS (EI) for C₂₆H₃₂N₆O₃S: 509 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-(1-methylethyl)pyridine-3-sulfonamide. Prepared by the method of example 5 using 2-amino-5-bromo-N-isopropylpyridine-3-sulfonamide (reagent preparation 25) in step 1. ¹H NMR (400 MHz, DMSO-d6) δ 8.49-8.43 (m, 1H), 8.37 (s, 1H), 8.05 (d, 1H), 7.76 (br s, 1H), 7.57 (d, 1H), 7.44 (dd, 1H), 7.02 (d, 1H), 6.66 (br s, 2H), 4.61 (s, 2H), 4.35-4.27 (m, 2H), 3.86-3.79 (m, 2H), 3.24-3.13 (m, 1H), 2.69 (t, 2H), 2.44 (s, 2H), 1.59 (t, 2H), 0.96 (d, 6H), 0.84 (s, 6H); MS (EI) for C₂₇H₃₄N₆O₃S: 523 (MH⁺).

2-amino-N-[2-(dimethylamino)ethyl]-5-[4-(6,6-dimethyl-5,6,7,8-tetrahydroquinazolin-4-yl)-2,3,4,5-tetrahydro-1,4-benzoxazepin-7-yl]pyridine-3-sulfonamide. Prepared by the method of example 5 using 2-amino-5-bromo-N-(2-(dimethylamino)ethyl)pyridine-3-sulfonamide (reagent preparation 25) in step 1. ¹H NMR (400 MHz, DMSO-d6) δ 8.42 (d, 1H), 8.36 (s, 1H), 8.02 (d, 1H), 7.72 (br s, 1H), 7.56 (d, 1H), 7.42 (dd, 1H), 7.01 (d, 1H), 6.69 (br s, 2H), 4.61 (s, 2H), 4.34-4.27 (m, 2H), 3.87-3.79 (m, 2H), 2.80 (t, 2H), 2.71 (t, 2H), 2.44 (s, 2H), 2.20 (t, 2H), 2.03 (s, 6H), 1.59 (t, 2H), 0.84 (s, 6H); MS (EI) for C₂₈H₃₇N₇O₃S: 552 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-(2-hydroxyethyl)pyridine-3-sulfonamide. Prepared by the method of example 5 using 2-amino-5-bromo-N-(2-hydroxyethyl)pyridine-3-sulfonamide (reagent preparation 25) in step 1. ¹H NMR (400 MHz, DMSO-d6) δ 8.48 (d, 1H), 8.37 (s, 1H), 8.04 (d, 1H), 7.87 (br s, 1H), 7.58 (d, 1H), 7.44 (dd, 1H), 7.02 (d, 1H), 6.69 (br s, 2H), 4.70 (br s, 1H), 4.62 (s, 2H), 4.36-4.27 (m, 2H), 3.87-3.80 (m, 2H), 3.36 (t, 2H), 2.80 (t, 2H), 2.71 (t, 2H), 2.44 (s, 2H), 1.60 (t, 2H), 0.84 (s, 6H); MS (EI) for C₂₆H₃₂N₆O₄S: 525 (MH⁺).

1-({2-amino-5-[4-(6,6-dimethyl-5,6,7,8-tetrahydroquinazolin-4-yl)-2,3,4,5-tetrahydro-1,4-benzoxazepin-7-yl]pyridin-3-yl}sulfonyl)-3-(hydroxymethyl)azetidin-3-ol. Prepared by the method of example 5 using 1-(2-amino-5-bromopyridin-3-ylsulfonyl)-3-(hydroxymethyl)azetidin-3-ol (reagent preparation 25) in step 1. ¹H NMR (400 MHz, DMSO-d6) δ 8.61 (d, 1H), 8.37 (s, 1H), 7.99 (d, 1H), 7.61 (d, 1H), 7.50 (dd, 1H), 7.03 (d, 1H), 6.79 (br s, 2H), 5.74 (br s, 1H), 4.94 (br s, 1H), 4.62 (s, 4.35-4.27 (m, 2H), 3.88-3.73 (m, 4H), 3.54 (d, 2H), 3.32 (s, 2H), 2.71 (t, 2H), 2.46 (s, 2H), 1.60 (t, 2H), 0.85 (s, 6H); MS (EI) for C₂₈H₃₄N₆O₅S: 567 (MH⁺).

N²-({2-amino-5-[4-(6,6-dimethyl-5,6,7,8-tetrahydroquinazolin-4-yl)-2,3,4,5-tetrahydro-1,4-benzoxazepin-7-yl]pyridin-3-yl}sulfonyl)glycinamide. Prepared by the method of example 5 using 2-(2-amino-5-bromopyridine-3-sulfonamido)acetamide (reagent preparation 25) in step 1. ¹H NMR (400 MHz, DMSO-d6) δ 8.50 (d, 1H), 8.37 (s, 1H), 8.08 (br s, 1H), 8.05 (d, 1H), 7.58 (d, 1H), 7.45 (dd, 1H), 7.31 (br s, 1H), 7.12 (br s, 1H), 7.02 (d, 1H), 6.75 (br s, 2H), 4.62 (s, 2H), 4.35-4.27 (m, 2H), 3.88-3.79 (m, 2H), 3.43 (s, 2H), 2.71 (t, 2H), 2.45 (s, 2H), 1.60 (t, 2H), 0.85 (s, 6H); MS (EI) for C₂₆H₃₁N₇O₄S: 538 (MH⁺).

(3R)-1-({2-amino-5-[4-(6,6-dimethyl-5,6,7,8-tetrahydroquinazolin-4-yl)-2,3,4,5-tetrahydro-1,4-benzoxazepin-7-yl]pyridin-3-yl}sulfonyl)pyrrolidin-3-ol. Prepared by the method of example 5 using (R)-1-(2-amino-5-bromopyridin-3-ylsulfonyl)pyrrolidin-3-ol (reagent preparation 25) in step 1. ¹H NMR (400 MHz, DMSO-d6) δ 8.54 (d, 1H), 8.37 (s, 1H), 8.00 (d, 1H), 7.59 (d, 1H), 7.47 (dd, 1H), 7.02 (d, 1H), 6.76 (s, 2H), 5.03 (d, 1H), 4.62 (s, 2H), 4.35-4.28 (m, 2H), 4.27-4.20 (m, 1H), 3.86-3.80 (m, 2H), 3.45-3.27 (m, 4H (buried)), 3.16-3.10 (m, 1H), 2.71 (t, 2H), 2.45 (s, 2H), 1.93-1.81 (m, 1H), 1.73 (s, 1H), 1.60 (t, 2H), 0.85 (s, 6H), 0.85 (s, 6H); MS (EI) for C₂₈H₃₄N₆O₄S: 551 (MH⁺).

(3R)-1-({2-amino-5-[4-(6,6-dimethyl-5,6,7,8-tetrahydroquinazolin-4-yl)-2,3,4,5-tetrahydro-1,4-benzoxazepin-7-yl]pyridin-3-yl}sulfonyl)pyrrolidin-3-ol. Prepared by the method of example 5 using tert-butyl 3-(2-amino-5-bromopyridine-3-sulfonamido)-2-hydroxypropylcarbamate (reagent preparation 25) in step 1 followed by Boc deprotection. ¹H NMR (400 MHz, DMSO-d6) δ 8.50 (d, 1H), 8.37 (s, 1H), 8.05 (d, 1H), 7.58 (d, 1H), 7.45 (dd, 1H), 7.02 (d, 1H), 6.71 (br s, 2H), 4.62 (s, 2H), 4.35-4.26 (m, 2H), 3.87-3.77 (m, 2H), 3.43-3.23 (m, 2H (buried)), 2.82 (dd, 1H), 2.75-2.61 (m, 3H), 2.47-2.37 (m, 3H), 1.60 (t, 2H), 0.84 (s, 6H); MS (EI) for C₂₇H₃₅N₇O₄S: 554 (MH⁺).

3-{[3-(dimethylamino)azetidin-1-yl]sulfonyl}-5-[4-(6,6-dimethyl-5,6,7,8-tetrahydroquinazolin-4-yl)-2,3,4,5-tetrahydro-1,4-benzoxazepin-7-yl]pyridin-2-amine. Prepared by the method of example 5 using 5-bromo-3-(3-(dimethylamino)azetidin-1-ylsulfonyl)pyridin-2-amine (reagent preparation 25) in step 1. ¹H NMR (400 MHz, DMSO-d6) δ 8.62 (d, 1H), 8.37 (s, 1H), 8.00 (d, 1H), 7.63 (d, 1H), 7.50 (dd, 1H), 7.03 (d, 1H), 6.82 (br s, 2H), 4.62 (s, 2H), 4.36-4.29 (m, 2H), 3.87-3.78 (m, 4H), 3.63-3.56 (m, 2H), 3.02 (p, 1H), 2.71 (t, 2H), 2.45 (s, 2H), 1.94 (s, 6H), 1.59 (t, 2H), 0.84 (s, 6H); MS (EI) for C₂₉H₃₇N₇O₃S: 564 (MH⁺).

5-[4-(6,6-dimethyl-5,6,7,8-tetrahydroquinazolin-4-yl)-2,3,4,5-tetrahydro-1,4-benzoxazepin-7-yl]-N-(2-hydroxyethyl)-2-(methylamino)pyridine-3-sulfonamide. Prepared by the method of example 5 using 5-bromo-N-(2-hydroxyethyl)-2-(methylamino)pyridine-3-sulfonamide (reagent preparation 25) in step 1. ¹H NMR (400 MHz, DMSO-d6) δ 8.60 (d, 1H), 8.37 (s, 1H), 8.06 (d, 1H), 7.85 (t, 1H), 7.58 (d, 1H), 7.45 (dd, 1H), 7.02 (d, 1H), 6.57 (q, 1H), 4.76 (t, 1H), 4.62 (s, 2H), 4.35-4.28 (m, 2H), 3.87-3.80 (m, 2H), 3.37 (q, 2H), 2.97 (d, 3H), 2.81 (q, 2H), 2.71 (t, 2H), 2.44 (s, 2H), 1.60 (t, 2H), 0.84 (s, 6H); MS (EI) for C₂₇H₃₄N₆O₄S: 539 (MH⁺).

5-[4-(6,6-dimethyl-5,6,7,8-tetrahydroquinazolin-4-yl)-2,3,4,5-tetrahydro-1,4-benzoxazepin-7-yl]-3-{[3-(methylamino)azetidin-1-yl]sulfonyl}pyridin-2-amine. Prepared by the method of example 5 using N-(1-(2-amino-5-bromopyridin-3-ylsulfonyl)azetidin-3-yl)-N-methyl-2-nitrobenzenesulfonamide (reagent preparation 25) in step 1 followed by 2-benzenesulfonyl-group deprotection. ¹H NMR (400 MHz, DMSO-d6) δ 8.61 (d, 1H), 8.37 (s, 1H), 7.98 (d, 1H), 7.62 (d, 1H), 7.49 (dd, 1H), 7.03 (d, 1H), 6.79 (br s, 2H), 4.62 (s, 2H), 4.35-4.29 (m, 2H), 3.90-3.80 (m, 4H), 3.54-3.45 (m, 2H), 3.43-3.35 (m, 1H), 2.71 (t, 2H), 2.45 (s, 2H), 2.07 (s, 3H), 1.60 (t, 2H), 0.85 (s, 6H); MS (EI) for C₂₈H₃₅N₇O₃S: 550 (MH⁺).

5-[4-(6,6-dimethyl-5,6,7,8-tetrahydroquinazolin-4-yl)-2,3,4,5-tetrahydro-1,4-benzoxazepin-7-yl]-3-(piperazin-1-ylsulfonyl)pyridin-2-amine. Prepared by the method of example 5 using tert-butyl 4-(2-amino-5-bromopyridin-3-ylsulfonyl)piperazine-1-carboxylate (reagent preparation 25) in step 1 followed by Boc deprotection. ¹H NMR (400 MHz, DMSO-d6) δ 8.57 (d, 1H), 8.37 (s, 1H), 7.92 (d, 1H), 7.60 (d, 1H), 7.47 (dd, 1H), 7.03 (d, 1H), 6.80 (br s, 2H), 4.62 (s, 2H), 4.36-4.29 (m, 2H), 3.87-3.78 (m, 2H), 2.99-2.90 (m, 4H), 2.75-2.65 (m, 6H), 2.44 (s, 2H), 1.59 (t, 2H), 0.84 (s, 6H); MS (EI) for MS (EI) for C₂₈H₃₅N₇O₃S: 550 (MH⁺).

N-{2-chloro-5-[4-(6,6-dimethyl-5,6,7,8-tetrahydroquinazolin-4-yl)-2,3,4,5-tetrahydro-1,4-benzoxazepin-7-yl]phenyl}methanesulfonamide. Prepared by the method of example 5 using N-(5-bromo-2-chlorophenyl)methanesulfonamide (reagent preparation 26) in step 1. ¹H NMR (400 MHz, DMSO-d6) δ 9.58 (s, 1H), 8.35 (s, 1H), 7.68-7.57 (m, 3H), 7.56-7.45 (m, 2H), 7.05 (d, 1H), 4.65 (s, 2H), 4.37-4.29 (m, 2H), 3.88-3.80 (m, 2H), 3.07 (s, 3H), 2.70 (t, 2H), 2.43 (s, 2H), 1.59 (t, 2H), 0.84 (s, 6H); MS (EI) for C₂₆H₂₉ClN₄O₃S: 513 (MH⁺).

3-[(3-amino-3-methylazetidin-1-yl)sulfonyl]-5-[4-(6,6-dimethyl-5,6,7,8-tetrahydroquinazolin-4-yl)-2,3,4,5-tetrahydro-1,4-benzoxazepin-7-yl]pyridin-2-amine. Prepared by the method of example 5 using 3-(3-amino-3-methylazetidin-1-ylsulfonyl)-5-bromopyridin-2-amine (reagent preparation 25) in step 1. ¹H NMR (400 MHz, DMSO-d6) δ 8.61 (d, 1H), 8.37 (s, 1H), 7.98 (d, 1H), 7.61 (d, 1H), 7.49 (dd, 1H), 7.03 (d, 1H), 6.78 (br s, 2H), 4.62 (s, 2H), 4.36-4.28 (m, 2H), 3.87-3.79 (m, 2H), 3.60-3.48 (m, 4H), 2.71 (t, 2H), 2.45 (s, 2H), 2.02 (s, 2H), 1.60 (t, 2H), 1.19 (s, 3H), 0.85 (s, 6H); MS (EI) for MS (EI) for C₂₈H₃₅N₇O₃S: 550 (MH⁺).

5-[4-(6,6-dimethyl-5,6,7,8-tetrahydroquinazolin-4-yl)-2,3,4,5-tetrahydro-1,4-benzoxazepin-7-yl]-3-[(methylsulfonyl)methyl]pyridin-2-amine. Prepared by the method of example 5 using 5-bromo-3-(methylsulfonylmethyl)pyridin-2-amine (reagent preparation 27) in step 1. ¹H NMR (400 MHz, DMSO-d6) δ 8.37 (s, 1H), 8.28 (d, 1H), 7.73 (d, 1H), 7.53 (d, 1H), 7.37 (dd, 1H), 7.01 (d, 1H), 6.22 (s, 2H), 4.60 (s, 2H), 4.47 (s, 2H), 4.34-4.25 (m, 2H), 3.87-3.80 (m, 2H), 2.95 (s, 3H), 2.71 (t, 2H), 2.45 (s, 2H), 1.60 (t, 2H), 0.86 (s, 6H); MS (EI) for C₂₆H₃₁N₅O₃S: 494 (MH⁺).

7-{6-chloro-5-[(methylsulfonyl)methyl]pyridin-3-yl}-4-(6,6-dimethyl-5,6,7,8-tetrahydroquinazolin-4-yl)-2,3,4,5-tetrahydro-1,4-benzoxazepine. Prepared by the method of example 5 using 5-bromo-2-chloro-3-(methylsulfonylmethyl)pyridine (reagent preparation 27) in step 1. ¹H NMR (400 MHz, DMSO-d6) δ 8.74 (d, 1H), 8.36 (s, 1H), 8.23 (d, 1H), 7.73 (d, 1H), 7.57 (dd, 1H), 7.10 (d, 1H), 4.72 (s, 2H), 4.68 (s, 2H), 4.40-4.33 (m, 2H), 3.88-3.81 (m, 2H), 3.11 (s, 3H), 2.71 (t, 2H), 2.42 (s, 2H), 1.59 (t, 2H), 0.84 (s, 6H); MS (EI) for C₂₆H₂₉ClN₄O₃S: 513 (MH⁺).

2-amino-N-(2-amino-1,1-dimethylethyl)-5-[4-(6,6-dimethyl-5,6,7,8-tetrahydroquinazolin-4-yl)-2,3,4,5-tetrahydro-1,4-benzoxazepin-7-yl]pyridine-3-sulfonamide. Prepared as an acetate salt by the method of example 5 using tert-butyl 2-(2-amino-5-bromopyridine-3-sulfonamido)-2-methylpropylcarbamate (reagent preparation 25) in step 1 followed by Boc deprotection. ¹H NMR (400 MHz, DMSO-d6) δ 8.49 (d, 1H), 8.37 (s, 1H), 8.08 (d, 1H), 7.56 (d, 1H), 7.44 (dd, 1H), 7.02 (d, 1H), 6.68 (br s, 2H), 4.61 (s, 2H), 4.35-4.27 (m, 2H), 3.87-3.79 (m, 2H), 2.71 (t, 2H), 2.44 (s, 2H), 2.42 (s, 2H), 1.89 (s, 4H), 1.60 (t, 2H), 1.02 (s, 6H), 0.84 (s, 6H); MS (EI) for C₂₈H₃₇N₇O₃S: 552 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-[(3aR,5r,6aS)-octahydrocyclopenta[c]pyrrol-5-ylmethyl]pyridine-3-sulfonamide. Prepared by the method of example 5 using tert-butyl 5-((2-amino-5-bromopyridine-3-sulfonamido)methyl)hexahydrocyclopenta[c]pyrrole-2(1H)-carboxylate (reagent preparation 25) in step 1 followed by Boc deprotection. ¹H NMR (400 MHz, DMSO-d6) δ 8.51 (d, 1H), 8.37 (s, 1H), 8.03 (d, 1H), 7.77 (br s, 1H), 7.58 (d, 1H), 7.44 (dd, 1H), 7.02 (d, 1H), 6.68 (br s, 2H), 4.62 (s, 2H), 4.36-4.26 (m, 2H), 3.88-3.77 (m, 2H), 2.79-2.63 (m, 3H), 2.54-2.34 (m, 9H), 1.95-1.82 (m, 3H), 1.82-1.70 (m, 1H), 1.59 (t, 2H), 0.82 (s, 6H), 0.80-0.67 (m, 2H); MS (EI) for C₃₂H₄₁N₇O₃S: 604 (MH⁺).

2-amino-N-(2-aminobutyl)-5-[4-(6,6-dimethyl-5,6,7,8-tetrahydroquinazolin-4-yl)-2,3,4,5-tetrahydro-1,4-benzoxazepin-7-yl]pyridine-3-sulfonamide. Prepared by the method of example 5 using tert-butyl 1-(2-amino-5-bromopyridine-3-sulfonamido)butan-2-ylcarbamate (reagent preparation 25) in step 1 followed by Boc deprotection. ¹H NMR (400 MHz, DMSO-d6) δ 8.50 (d, 1H), 8.37 (s, 1H), 8.04 (d, 1H), 7.58 (d, 1H), 7.45 (dd, 1H), 7.02 (d, 1H), 6.70 (br s, 2H), 4.62 (s, 2H), 4.35-4.27 (m, 2H), 3.86-3.79 (m, 2H), 2.75-2.65 (m, 4H), 2.62-2.54 (m, 1H), 2.48-2.41 (m, 3H), 1.64-1.55 (m, 4H), 1.39-1.25 (m, 1H), 1.16-1.03 (m, 1H), 0.84 (s, 6H), 0.76 (t, 3H); MS (EI) for MS (EI) for C₂₈H₃₇N₇O₃S: 552 (MH⁺).

N-{5-[4-(6,6-dimethyl-5,6,7,8-tetrahydroquinazolin-4-yl)-2,3,4,5-tetrahydro-1,4-benzoxazepin-7-yl]-2-methylpyridin-3-yl}methanesulfonamide. Prepared by the method of example 5 using N-(5-bromo-2-methylpyridin-3-yl)methanesulfonamide (reagent preparation 26) in step 1. ¹H NMR (400 MHz, DMSO-d6) δ 8.59 (d, 1H), 8.36 (s, 1H), 7.82 (d, 1H), 7.66 (d, 1H), 7.52 (dd, 1H), 7.07 (d, 1H), 4.65 (s, 2H), 4.39-4.29 (m, 2H), 3.90-3.79 (m, 2H), 3.07 (s, 3H), 2.71 (t, 2H), 2.53 (s, 3H), 2.44 (s, 2H), 1.60 (t, 2H), 0.85 (s, 6H); MS (EI) for C₂₆H₃₁N₅O₃S: 494 (MH⁺).

2-amino-N-(3-amino-3-methylbutyl)-5-[4-(6,6-dimethyl-5,6,7,8-tetrahydroquinazolin-4-yl)-2,3,4,5-tetrahydro-1,4-benzoxazepin-7-yl]pyridine-3-sulfonamide. Prepared by the method of example 5 using tert-butyl 4-(2-amino-5-bromopyridine-3-sulfonamido)-2-methylbutan-2-ylcarbamate (reagent preparation 25) in step 1 followed by Boc deprotection. ¹H NMR (400 MHz, DMSO-d6) δ 8.51 (d, 1H), 8.37 (s, 1H), 8.04 (d, 1H), 7.58 (d, 1H), 7.44 (dd, 1H), 7.02 (d, 1H), 6.66 (br s, 2H), 4.62 (s, 2H), 4.36-4.27 (m, 2H), 3.87-3.79 (m, 2H), 2.89-2.80 (m, 2H), 2.71 (t, 2H), 2.45 (s, 2H), 1.59 (t, 2H), 1.43-1.34 (m, 2H), 0.90 (s, 6H), 0.84 (s, 6H); MS (EI) for C₂₉H₃₉N₇O₃S: 566 (MH⁺).

N-{2-chloro-5-[4-(6,6-dimethyl-5,6,7,8-tetrahydroquinazolin-4-yl)-2,3,4,5-tetrahydro-1,4-benzoxazepin-7-yl]pyridin-3-yl}-N-methylmethanesulfonamide. Prepared by the method of example 5 using N-(5-bromo-2-chloropyridin-3-yl)-N-methylmethanesulfonamide (reagent preparation 28) in step 1. ¹H NMR (400 MHz, DMSO-d6) δ 8.70 (s, 1H), 8.34 (s, 1H), 8.28 (s, 1H), 7.79 (s, 1H), 7.64 (d, 1H), 7.05 (d, 1H), 4.65 (s, 2H), 4.40-4.24 (m, 2H), 3.87-3.76 (m, 2H), 3.23 (s, 3H), 3.21 (s, 3H), 2.75-2.61 (m, 2H), 2.41 (s, 2H), 1.64-1.48 (m, 2H), 0.81 (s, 6H); MS (EI) for C₂₆H₃₀ClN₅O₃S: 528 (MH⁺).

7-{6-chloro-5-[(difluoromethyl)oxy]pyridin-3-yl}-4-(6,6-dimethyl-5,6,7,8-tetrahydroquinazolin-4-yl)-2,3,4,5-tetrahydro-1,4-benzoxazepine. Prepared by the method of example 5 using 5-bromo-2-chloro-3-(difluoromethoxy)pyridine (reagent preparation 29) in step 1. ¹H NMR (400 MHz, DMSO-d6) δ 8.64 (d, 1H), 8.36 (s, 1H), 8.08 (d, 1H), 7.81 (d, 1H), 7.66 (dd, 1H), 7.47 (t, 1H), 7.09 (d, 1H), 4.67 (s, 2H), 4.40-4.33 (m, 2H), 3.88-3.80 (m, 2H), 2.71 (t, 2H), 2.42 (s, 2H), 1.59 (t, 2H), 0.83 (s, 6H); MS (EI) for C₂₅H₂₅P₂ClN₄O₂: 487 (MH⁺).

N-{5-[4-(6,6-dimethyl-5,6,7,8-tetrahydroquinazolin-4-yl)-2,3,4,5-tetrahydro-1,4-benzoxazepin-7-yl]-2-(methyloxy)pyridin-3-yl}methanesulfonamide. Prepared by the method of example 5 using N-(5-bromo-2-methoxypyridin-3-yl)methanesulfonamide (reagent preparation 26) in step 1. ¹H NMR (400 MHz, DMSO-d6) δ 9.33 (s, 1H), 8.36 (s, 1H), 8.27 (d, 1H), 7.83 (d, 1H), 7.59 (d, 1H), 7.46 (dd, 1H), 7.04 (d, 1H), 4.64 (s, 2H), 4.36-4.28 (m, 2H), 3.95 (s, 3H), 3.88-3.79 (m, 2H), 3.07 (s, 3H), 2.71 (t, 2H), 2.44 (s, 2H), 1.60 (t, 2H), 0.85 (s, 6H); MS (EI) for C₂₆H₃₁N₅O₄S: 510 (MH⁺).

N-{5-[4-(6,6-dimethyl-5,6,7,8-tetrahydroquinazolin-4-yl)-2,3,4,5-tetrahydro-1,4-benzoxazepin-7-yl]-2-(ethyloxy)pyridin-3-yl}methanesulfonamide. Prepared by the method of example 5 using N-(5-bromo-2-ethoxypyridin-3-yl)methanesulfonamide (reagent preparation 30) in step 1. ¹H NMR (400 MHz, DMSO-d6) δ 9.27 (br s, 1H), 8.36 (s, 1H), 8.24 (d, 1H), 7.81 (d, 1H), 7.59 (d, 1H), 7.45 (dd, 1H), 7.04 (d, 1H), 4.63 (s, 2H), 4.40 (q, 2H), 4.36-4.29 (m, 2H), 3.88-3.80 (m, 2H), 3.06 (s, 3H), 2.71 (t, 2H), 2.44 (s, 2H), 1.60 (t, 2H), 1.37 (t, 3H), 0.85 (s, 6H); MS (EI) for C₂₇H₃₃N₅O₄S: 524 (MH⁺).

3-amino-5-[4-(6,6-dimethyl-5,6,7,8-tetrahydroquinazolin-4-yl)-2,3,4,5-tetrahydro-1,4-benzoxazepin-7-yl]pyridine-2-carboxamide. Prepared by the method of example 5 using 3-amino-5-bromopicolinonitrile in step 1. The nitrile substituent hydrolyzes to the carboxamide under the aqueous reaction conditions for the coupling. ¹H NMR (400 MHz, DMSO-d6) δ 8.36 (s, 1H), 8.06 (d, 1H), 7.93 (d, 1H), 7.65 (d, 1H), 7.48 (dd, 1H), 7.37-7.31 (m, 2H), 7.06 (d, 1H), 6.90 (br s, 2H), 4.66 (s, 2H), 4.38-4.32 (m, 2H), 3.87-3.81 (m, 2H), 2.71 (t, 2H), 2.41 (s, 2H), 1.59 (t, 2H), 0.83 (s, 6H); MS (EI) for C₂₅H₂₈N₆O₂: 445 (MH⁺).

N-{2-amino-5-[4-(6,6-dimethyl-5,6,7,8-tetrahydroquinazolin-4-yl)-2,3,4,5-tetrahydro-1,4-benzoxazepin-7-yl]pyridin-3-yl}methanesulfonamide. Prepared as an acetate salt by the method of example 5 using N-(2-amino-5-bromopyridin-3-yl)methanesulfonamide (reagent preparation 31) in step 1 . ¹H NMR (400 MHz, DMSO-d6) δ 8.36 (s, 1H), 8.13 (d, 1H), 7.60 (d, 1H), 7.51 (d, 1H), 7.37 (dd, 1H), 7.00 (d, 1H), 6.07 (br s, 2H), 4.60 (s, 2H), 4.32-4.26 (m, 2H), 3.87-3.79 (m, 2H), 2.97 (s, 3H), 2.71 (t, 2H), 2.45 (s, 2H), 1.91 (s, 3H), 1.60 (t, 2H), 0.86 (s, 6H); MS (EI) for C₂₅H₃₀N₆O₃S; 495 (MH⁺).

N-{2-cyano-5-[4-(6,6-dimethyl-5,6,7,8-tetrahydroquinazolin-4-yl)-2,3,4,5-tetrahydro-1,4-benzoxazepin-7-yl]pyridin-3-yl}methanesulfonamide. Prepared as an acetate salt by the method of example 5 using N-(5-bromo-2-cyanopyridin-3-yl)methanesulfonamide (reagent preparation 26) in step 1. This coupling was completed using water free conditions to prevent nitrile hydrolysis. ¹H NMR (400 MHz, DMSO-d6) δ 8.68 (br s, 1H), 8.37 (s, 1H), 8.03 (s, 1H), 7.77 (s, 1H), 7.61 (d, 1H), 7.09 (d, 1H), 4.71 (s, 2H), 4.42-4.35 (m, 2H), 3.91-3.83 (m, 2H), 3.09 (br s, 3H), 2.71 (t, 2H), 2.42 (s, 2H), 1.91 (s, 1H), 1.59 (t, 2H), 0.82 (d, 6H); MS (EI) for C₂₆H₂₈N₆O₃S: 505 (MH⁺).

N-{5-[4-(6,6-dimethyl-5,6,7,8-tetrahydroquinazolin-4-yl)-2,3,4,5-tetrahydro-1,4-benzoxazepin-7-yl]-2-[(phenylmethyl)oxy]pyridin-3-yl}methanesulfonamide. Prepared by the method of example 5 using N-(2-(benzyloxy)-5-bromopyridin-3-yl)methanesulfonamide (reagent preparation 30) in step 1. ¹H NMR (400 MHz, DMSO-d6) δ 9.41 (br s, 1H), 8.36 (s, 1H), 8.27 (d, 1H), 7.85 (d, 1H), 7.60 (d, 1H), 7.57-7.52 (m, 2H), 7.47 (dd, 1H), 7.43-7.36 (m, 2H), 7.36-7.30 (m, 1H), 7.04 (d, 1H), 5.45 (s, 2H), 4.64 (s, 2H), 4.35-4.29 (m, 2H), 3.88-3.80 (m, 2H), 3.01 (s, 3H), 2.71 (t, 2H), 2.44 (s, 2H), 1.60 (t, 2H), 0.85 (s, 6H); MS (EI) for C₃₂H₃₅N₅O₄S: 586 (MH⁺).

2-amino-N-(2-amino-2-methylpropyl)-5-[4-(6,6-dimethyl-5,6,7,8-tetrahydroquinazolin-4-yl)-2,3,4,5-tetrahydro-1,4-benzoxazepin-7-yl]pyridine-3-sulfonamide. Prepared by the method of example 5 using 2-amino-N-(2-amino-2-methylpropyl)-5-bromopyridine-3-sulfonamide (reagent preparation 25) in step 1. ¹H NMR (400 MHz, DMSO-d6) δ 8.45 (br s, 1H), 8.37 (s, 1H), 8.02 (d, 1H), 7.57 (s, 1H), 7.43 (dd, 1H), 7.02 (d, 1H), 6.71 (br s, 2H), 4.62 (s, 2H), 4.35-4.28 (m, 2H), 3.86-3.80 (m, 2H), 2.71 (t, 2H), 2.54 (s, 2H), 2.44 (s, 2H), 1.59 (t, 2H), 0.92 (s, 6H), 0.84 (s, 6H); MS (EI) for C₂₈H₃₇N₇O₃S: 552 (MH⁺).

3-[(3-aminoazetidin-1-yl)sulfonyl]-5-[4-(6,6-dimethyl-5,6,7,8-tetrahydroquinazolin-4-yl)-2,3,4,5-tetrahydro-1,4-benzoxazepin-7-yl]pyridin-2-amine. Prepared by the method of example 5 using tert-butyl 1-(2-amino-5-bromopyridin-3-ylsulfonyl)azetidin-3-ylcarbamate (reagent preparation 25) in step 1 followed by Boc deprotection. ¹H NMR (400 MHz, DMSO-d6) δ 8.61 (d, 1H), 8.37 (s, 1H), 7.98 (d, 1H), 7.61 (d, 1H), 7.49 (dd, 1H), 7.03 (d, 1H), 6.77 (br s, 2H), 4.62 (s, 2H), 4.36-4.29 (m, 2H), 3.91-3.80 (m, 4H), 3.57 (p, 1H), 3.39 (t, 2H), 2.71 (t, 2H), 2.45 (s, 2H), 1.60 (t, 2H), 0.85 (s, 6H); MS (EI) for C₂₇H₃₃N₇O₃S: 536 (MH⁺).

N-{2-chloro-5-[4-(6,6-dimethyl-5,6,7,8-tetrahydroquinazolin-4-yl)-2,3,4,5-tetrahydro-1,4-benzoxazepin-7-yl]pyridin-3-yl}methanesulfonamide. Prepared by the method of example 5 using N-(5-bromo-2-chloropyridin-3-yl)methanesulfonamide (reagent preparation 24) in step 1. ¹H NMR (400 MHz, DMSO-d6) δ 9.86 (br s, 1H), 8.56 (d, 1H), 8.36 (s, 1H), 8.04 (d, 1H), 7.72 (d, 1H), 7.57 (dd, 1H), 7.08 (d, 1H), 4.67 (s, 2H), 4.39-4.32 (m, 2H), 3.89-3.81 (m, 2H), 3.16 (s, 3H), 2.71 (t, 2H), 2.43 (s, 2H), 1.59 (t, 2H), 0.84 (s, 6H); MS (EI) for C₂₅H₂₈ClN₅O₃S: 514 (MH⁺).

1-{6-[4-(6,6-dimethyl-5,6,7,8-tetrahydroquinazolin-4-yl)-2,3,4,5-tetrahydro-1,4-benzoxazepin-7-yl]-3H-imidazo[4,5-b]pyridin-2-yl}ethanol. Prepared according to the method of example 5 by using 1-(6-bromo-3H-imidazo[4,5-b]pyridin-2-yl)ethanol (reagent preparation 19) and [4-(6,6-dimethyl-5,6,7,8-tetrahydroquinazolin-4-yl)-2,3,4,5-tetrahydro-1,4-benzoxazepin-7-yl]boronic acid (reagent preparation 23) in step 1. ¹H NMR (400 MHz, Methanol-d₄): 8.56 (br, 1H), 8.34 (s, 1H), 8.11 (br, 1H), 7.61 (s, 1H), 7.54 (d, 1H), 7.10 (d, 1H), 5.11 (m, 1H), 4.73 (s, 2H), 4.37 (m, 2H), 3.95 (m, 2H), 2.79 (t, 2H), 2.48 (s, 2H), 1.68 (t, 2H), 1.64 (d, 3H), 0.88 (s, 6H); MS (EI) for C₂₇H₃₀N₆O₂: 471 (MH⁺).

4-(6,6-dimethyl-5,6,7,8-tetrahydroquinazolin-4-yl)-7-[5-(methyloxy)pyridin-3-yl]-2,3,4,5-tetrahydro-1,4-benzoxazepine. Prepared according to the method of example 5 by using 3-bromo-5-methoxypyridine and [4-(6,6-dimethyl-5,6,7,8-tetrahydroquinazolin-4-yl)-2,3,4,5-tetrahydro-1,4-benzoxazepin-7-yl]boronic acid (reagent preparation 23) in step 1. ¹H NMR (400 MHz, DMSO-d₆): 8.49 (s, 1H), 8.40 (s, 1H), 8.26 (s, 1H), 7.75 (s, 1H), 7.59 (m, 2H), 7.07 (d, 1H), 4.65 (s, 2H), 4.35 (m, 2H), 3.90 (s, 3H), 3.84 (m, 2H), 2.67 (t, 2H), 2.44 (s, 2H), 1.57 (t, 2H), 0.82 (s, 6H); MS (EI) for C₂₅H₂₈N₄O₂: 417 (MH⁺).

7-[4-(6,6-dimethyl-5,6,7,8-tetrahydroquinazolin-4-yl)-2,3,4,5-tetrahydro-1,4-benzoxazepin-7-yl]-2H-pyrido[3,2-b][1,4]oxazin-3(4H)-one. Prepared according to the method of example 5 by using 7-bromo-2H-pyrido[3,2-b][1,4]oxazin-3(4H)-one and [4-(6,6-dimethyl-5,6,7,8-tetrahydroquinazolin-4-yl)-2,3,4,5-tetrahydro-1,4-benzoxazepin-7-yl]boronic acid (reagent preparation 23) in step 1. ¹H NMR (400 MHz, DMSO-d₆): 8.38 (s, 1H), 8.21 (s, 1H), 7.70 (s, 1H), 7.62 (s, 1H), 7.53 (d, 1H), 7.07 (d, 1H), 4.68 (s, 2H), 4.62 (s, 2H), 4.33 (m, 2H), 3.82 (m, 2H), 2.71 (t, 2H), 2.45 (s, 2H), 1.56 (t, 2H), 0.84 (s, 6H); MS (EI) for C₂₆H₂₇N₅O₃: 458 (MH⁺).

4-(6,6-dimethyl-5,6,7,8-tetrahydroquinazolin-4-yl)-7-pyrido[2,3-b]pyrazin-7-yl-2,3,4,5-tetrahydro-1,4-benzoxazepine. Prepared according to the method of example 5 by using 7-bromopyrido[2,3-b]pyrazine and [4-(6,6-dimethyl-5,6,7,8-tetrahydroquinazolin-4-yl)-2,3,4,5-tetrahydro-1,4-benzoxazepin-7-yl]boronic acid (reagent preparation 23) in step 1. ¹H NMR (400 MHz, DMSO-d₆): 9.56 (s, 1H), 9.13 (s, 1H), 9.10 (s, 1H), 8.78 (s, 1H), 8.39 (s, 1H), 8.05 (s, 1H), 7.88 (d, 1H), 7.14 (d, 1H), 4.71 (s, 2H), 4.41 (m, 2H), 3.85 (m, 2H), 2.71 (t, 2H), 2.49 (s, 2H), 1.62 (t, 2H), 0.84 (s, 6H); MS (EI) for C₂₆H₂₆N₆O: 439 (MH⁺).

1-{6-[4-(6,6-dimethyl-5,6,7,8-tetrahydroquinazolin-4-yl)-2,3,4,5-tetrahydro-1,4-benzoxazepin-7-yl]-1H-imidazo[4,5-b]pyridin-2-yl}-N-methylmethanamine. Prepared according to the method of example 5 by using phenylmethyl{[6-bromo-3-({[2-(trimethylsilyl)ethyl]oxy}methyl)-3H-imidazo[4,5-b]pyridin-2-yl]methyl}methylcarbamate (reagent preparation 19) and [4-(6,6-dimethyl-5,6,7,8-tetrahydroquinazolin-4-yl)-2,3,4,5-tetrahydro-1,4-benzoxazepin-7-yl]boronic acid (reagent preparation 23) in step 1. ¹H NMR (400 MHz, Methanol-d₄): 8.62 (br, 1H), 8.35 (s, 1H), 8.10 (s, 1H), 7.62 (br, 1H), 7.53 (d, 1H), 7.09 (d, 1H), 4.74 (s, 2H), 4.38 (m, 2H), 4.22 (s, 2H), 3.97 (m, 2H), 2.77 (t, 2H), 2.59 (s, 3H), 2.48 (s, 2H), 1.68 (t, 2H), 0.88 (s, 6H); MS (EI) for C₂₇H₃₁N₇O: 470 (MH⁺).

1-{6-[4-(6,6-dimethyl-5,6,7,8-tetrahydroquinazolin-4-yl)-2,3,4,5-tetrahydro-1,4-benzoxazepin-7-yl]-1H-imidazo[4,5-b]pyridin-2-yl}-N,N-dimethylmethanamine. Prepared according to the method of example 5 by using phenylmethyl({6-[4-(6,6-dimethyl-5,6,7,8-tetrahydroquinazolin-4-yl)-2,3,4,5-tetrahydro-1,4-benzoxazepin-7-yl]-3-({[2-(trimethylsilyl)ethyl]oxy}methyl)-3H-imidazo[4,5-b]pyridin-2-yl}methyl)methylcarbamate in step 2. ¹H NMR (400 MHz, Methanol-d₄): 8.58 (s, 1H), 8.34 (s, 1H), 8.11 (s, 1H), 7.62 (br, 1H), 7.52 (d, 1H), 7.10 (d, 1H), 4.74 (s, 2H), 4.35 (m, 2H), 3.96 (m, 2H), 3.81 (s, 2H), 2.77 (t, 2H), 2.48 (s, 2H), 2.39 (s, 6H), 1.77 (t, 2H), 0.90 (s, 6H); MS (EI) for C₂₈H₃₃N₇O: 484 (MH⁺).

Phenylmethyl[(1S)-1-(6-{4-[(7S)-7-ethyl-2-methyl-5,6,7,8-tetrahydroquinazolin-4-yl]-2,3,4,5-tetrahydro-1,4-benzoxazepin-7-yl}-1H-imidazo[4,5-b]pyridin-2-yl)ethyl]carbamate. Prepared according to the method of example 5 by using phenylmethyl {(1S)-1-[6-bromo-3-({[2-(trimethylsilyl)ethyl]oxy}methyl)-3H-imidazo[4,5-b]pyridin-2-yl]ethyl}({[2-(trimethylsilyl)ethyl]oxy}methyl)carbamate (reagent preparation 19) and {4-[(7S)-7-ethyl-2-methyl-5,6,7,8-tetrahydroquinazolin-4-yl]-2,3,4,5-tetrahydro-1,4-benzoxazepin-7-yl}boronic acid (reagent preparation 23) in step 1. ¹H NMR (400 MHz, Methanol-d₄): 8.54 (br, 1H), 8.05 (br, 1H), 7.61 (s, 1H), 7.48 (d, 1H), 7.41 to 7.29 (m, 5H), 7.09 (d, 1H), 4.77 (d, 2H), 4.61 (d, 2H), 4.43 (m, 1H), 4.20 (m, 1H), 4.04 (m, 1H), 3.95 (m, 1H), 3.88 (s, 3H), 3.29 (m, 1H), 2.87 (m, 1H), 2.78 (m, 1H), 2.60 (m, 1H), 2.35 (s, 3H), 2.29 (m, 1H), 1.96 (m, 1H), 1.69 (m, 1H), 1.63 (d, 3H), 1.41 (m, 2H), 1.16 (m, 1H), 0.99 (t, 3H); MS (EI) for C₃₆H₃₉N₇O₃: 618 (MH⁺).

Phenylmethyl[(1S)-1-{6-[4-(6,6-dimethyl-5,6,7,8-tetrahydroquinazolin-4-yl)-2,3,4,5-tetrahydro-1,4-benzoxazepin-7-yl]-1H-imidazo[4,5-b]pyridin-2-yl}ethyl]carbamate. Prepared according to the method of example 5 by using phenylmethyl{(1S)-1-[6-bromo-3-({[2-(trimethylsilyl)ethyl]oxy}methyl)-3H-imidazo[4,5-b]pyridin-2-yl]ethyl}({[2-(trimethylsilyl)ethyl]oxy}methyl)carbamate (reagent preparation 19) and [4-(6,6-dimethyl-5,6,7,8-tetrahydroquinazolin-4-yl)-2,3,4,5-tetrahydro-1,4-benzoxazepin-7-yl]boronic acid (reagent preparation 23) in step 1. ¹H NMR (400 MHz, Methanol-d₄): 8.56 (br, 1H), 8.36 (br, 1H), 8.09 (br, 1H), 7.60 (s, 1H), 7.51 (d, 1H), 7.42 to 7.25 (m, 5H), 7.10 (d, 1H), 5.17 to 5.03 (dd, 2H), 4.73 (s, 2H), 4.61 (m, 1H), 4.37 (m, 1H), 3.95 (m, 1H), 2.79 (t, 2H), 2.48 (s, 2H), 1.72 to 1.56 (m, 5H), 0.90 (s, 6H); MS (EI) for C₃₅H₃₇N₇O₃: 604 (MH⁺).

(1S)-1-(6-{4-[(7S)-7-ethyl-5,6,7,8-tetrahydroquinazolin-4-yl]-2,3,4,5-tetrahydro-1,4-benzoxazepin-7-yl}-1H-imidazo[4,5-b]pyridin-2-yl)ethanamine. Prepared according to the method of example 5 by using phenylmethyl{(1S)-1-[6-bromo-3-({[2-(trimethylsilyl)ethyl]oxy}methyl)-3H-imidazo[4,5-b]pyridin-2-yl]ethyl}({[2-(trimethylsilyl)ethyl]oxy}methyl)carbamate (reagent preparation 19) and {4-[(7S)-7-ethyl-5,6,7,8-tetrahydroquinazolin-4-yl]-2,3,4,5-tetrahydro-1,4-benzoxazepin-7-yl}boronic acid (reagent preparation 23) in step 1. ¹H NMR (400 MHz, Methanol-d₄): 8.56 (s, 1H), 8.31 (s, 1H), 8.15 (s, 1H), 7.59 (s, 1H), 7.50 (d, 1H), 7.07 (d, 1H), 4.80 (m, 2H), 4.60 (q, 1H), 4.44 (m, 1H), 4.26 (m, 1H), 4.07 to 3.91 (m, 2H), 2.96 to 2.81 (m, 2H), 2.60 (m, 1H), 2.33 (m, 1H), 1.98 (m, 1H), 1.73 (m, 1H), 1.68 (d, 3H), 1.41 (m, 2H), 1.16 (m, 1H), 0.99 (t, 3H); MS (EI) for C₂₇H₃₁N₇O: 470 (MH⁺).

(1R)-1-(6-{4-[(7S)-7-ethyl-5,6,7,8-tetrahydroquinazolin-4-yl]-2,3,4,5-tetrahydro-1,4-benzoxazepin-7-yl}-1H-imidazo[4,5-b]pyridin-2-yl)ethanamine. Prepared according to the method of example 5 by using phenylmethyl{(1R)-1-[6-bromo-3-({[2-(trimethylsilyl)ethyl]oxy}methyl)-3H-imidazo[4,5-b]pyridin-2-yl]ethyl}({[2-(trimethylsilyl)ethyl]oxy}methyl)carbamate (reagent preparation 19) and {4-[(7S)-7-ethyl-5,6,7,8-tetrahydroquinazolin-4-yl]-2,3,4,5-tetrahydro-1,4-benzoxazepin-7-yl}boronic acid (reagent preparation 23) in step 1. ¹H NMR (400 MHz, Methanol-d₄): 8.59 (s, 1H), 8.31 (s, 1H), 8.12 (s, 1H), 7.61 (s, 1H), 7.50 (d, 1H), 7.09 (d, 1H), 4.80 (m, 2H), 4.53 (m, 1H), 4.43 (m, 1H), 4.27 (m, 1H), 4.06 to 3.93 (m, 2H), 2.96 to 2.78 (m, 2H), 2.62 (m, 1H), 2.32 (m, 1H), 1.95 (m, 1H), 1.75 (m, 1H), 1.67 (d, 3H), 1.41 (m, 2H), 1.16 (m, 1H), 0.99 (t, 3H); MS (EI) for C₂₇H₃₁N₇O: 470 (MH⁺).

3-[(3-amino-3-methylpyrrolidin-1-yl)sulfonyl]-5-{4-[(7S)-7-ethyl-2-methyl-5,6,7,8-tetrahydroquinazolin-4-yl]-2,3,4,5-tetrahydro-1,4-benzoxazepin-7-yl}pyridin-2-amine. Prepared according to the method of example 5 by using 1,1-dimethylethyl{1-[(2-amino-5-bromopyridin-3-yl)sulfonyl]-3-methylpyrrolidin-3-yl}carbamate (reagent preparation 25) and {4-[(7S)-7-ethyl-2-methyl-5,6,7,8-tetrahydroquinazolin-4-yl]-2,3,4,5-tetrahydro-1,4-benzoxazepin-7-yl}boronic acid (reagent preparation 23) in step 1. ¹H NMR (400 MHz, Methanol-d₄): 8.37 (s, 1H), 7.97 (s, 1H), 7.41 (s, 1H), 7.29 (d, 1H), 6.94 (d, 1H), 4.64 (m, 2H), 4.27 (m, 1H), 4.05 (m, 1H), 3.94 (m, 2H), 3.47 (m, 1H), 3.31 (m, 1H), 3.12 (m, 2H), 2.81 to 2.66 (m, 2H), 2.49 (m, 1H), 2.28 (s, 3H), 2.21 (m, 1H), 1.90 (m, 1H), 1.84 (m, 2H), 1.62 (m, 1H), 1.36 (m, 2H), 1.14 (s, 3H), 1.09 (m, 1H), 0.99 (t, 3H); MS (EI) for C₃₀H₃₉N₇O₃S: 578 (MH⁺).

3-{[(3S)-3-aminopyrrolidin-1-yl]sulfonyl}-5-[4-(6,6-dimethyl-5,6,7,8-tetrahydroquinazolin-4-yl)-2,3,4,5-tetrahydro-1,4-benzoxazepin-7-yl]pyridin-2-ol. Prepared according to the method of example 5 by using 1,1-dimethylethyl{(3S)-1-[(5-bromo-2-hydroxypyridin-3-yl)sulfonyl]pyrrolidin-3-yl}carbamate (reagent preparation 40) and [4-(6,6-dimethyl-5,6,7,8-tetrahydroquinazolin-4-yl)-2,3,4,5-tetrahydro-1,4-benzoxazepin-7-yl]boronic acid (reagent preparation 23) in step 1. ¹H NMR (400 MHz, Methanol-d₄): 8.53 (d, 2H), 8.00 (s, 1H), 7.57 (s, 1H), 7.41 (d, 1H), 7.03 (d, 1H), 5.12 (s, 2H), 4.45 (m, 1H), 4.29 (m, 1H), 3.93 (m, 1H), 3.72 to 3.55 (m, 6H), 2.85 (t, 2H), 2.56 (s, 2H), 2.35 (m, 1H), 2.06 (m, 1H), 1.69 (t, 2H), 0.93 (s, 6H); MS (EI) for C₂₈H₃₄N₆O₄S: 551 (MH⁺).

(3S)-1-({2-[(3S)-3-aminopyrrolidin-1-yl]-5-[4-(6,6-dimethyl-5,6,7,8-tetrahydroquinazolin-4-yl)-2,3,4,5-tetrahydro-1,4-benzoxazepin-7-yl]pyridin-3-yl}sulfonyl)pyrrolidin-3-amine. Prepared according to the method of example 5 by using 1,1-dimethylethyl[(3S)-1-({5-bromo-2-[(3S)-3-({[(1,1-dimethylethyl)oxy]carbonyl}amino)pyrrolidin-1-yl]pyridin-3-yl}sulfonyl)pyrrolidin-3-yl]carbamate (reagent preparation 40) and [4-(6,6-dimethyl-5,6,7,8-tetrahydroquinazolin-4-yl)-2,3,4,5-tetrahydro-1,4-benzoxazepin-7-yl]boronic acid (reagent preparation 23) in step 1. ¹H NMR (400 MHz, Methanol-d₄): 8.56 (s, 1H), 8.55 (s, 1H), 8.53 (s, 1H), 7.74 (s, 1H), 7.52 (d, 1H), 7.01 (d, 1H), 5.12 (s, 2H), 4.47 (m, 2H), 4.32 (m, 3H), 4.22 (m, 1H), 4.05 (m, 2H), 3.85 (m, 1H), 3.74 to 3.56 (m, 5H), 2.85 (t, 2H), 2.59 (s, 2H), 2.35 (m, 2H), 2.22 (m, 2H), 1.70 (t, 2H), 0.93 (s, 6H); MS (EI) for C₃₂H₄₂N₈O₃S: 619 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-[(3R)-1-methylpyrrolidin-3-yl]pyridine-3-sulfonamide. Prepared according to the method of example 5 by using 2-amino-5-bromo-N-[(3R)-1-methylpyrrolidin-3-yl]pyridine-3-sulfonamide (reagent preparation 25) and [4-(6,6-dimethyl-5,6,7,8-tetrahydroquinazolin-4-yl)-2,3,4,5-tetrahydro-1,4-benzoxazepin-7-yl]boronic acid (reagent preparation 23) in step 1. ¹H NMR (400 MHz, Methanol-d₄): 8.45 (s, 1H), 8.34 (s, 1H), 8.17 (s, 1H), 7.50 (s, 1H), 7.42 (d, 1H), 7.04 (d, 1H), 4.71 (s, 2H), 4.33 (m, 2H), 3.96 (m, 2H), 3.84 (m, 1H), 2.94 (m, 1H), 2.83 to 2.69 (m, 4H), 2.61 (m, 1H), 2.47 (s, 2H), 2.45 (s, 3H), 1.74 to 1.65 (m, 3H), 0.91 (s, 6H); MS (EI) for C₂₉H₃₇H₇O₃S: 564 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-{[(3S)-1-methylpyrrolidin-3-yl]methyl}pyridine-3-sulfonamide. Prepared according to the method of example 5 by using 2-amino-5-bromo-N-{[(3S)-1-methylpyrrolidin-3-yl]methyl}pyridine-3-sulfonamide (reagent preparation 25) and [4-(6,6-dimethyl-5,6,7,8-tetrahydroquinazolin-4-yl)-2,3,4,5-tetrahydro-1,4-benzoxazepin-7-yl]boronic acid (reagent preparation 23) in step 1. ¹H NMR (400 MHz, Methanol-d₄): 8.44 (br, 1H), 8.34 (s, 1H), 8.17 (s, 1H), 7.50 (s, 1H), 7.40 (d, 1H), 7.05 (d, 1H), 4.70 (s, 2H), 4.31 (m, 2H), 3.97 (m, 2H), 3.16 (m, 1H), 3.04 (t, 2H), 2.94 (d, 2H), 2.67 (t, 2H), 2.67 (s, 3H), 2.47 (s, 2H), 2.52 (m, 1H), 2.10 (m, 1H), 1.90 (m, 1H), 1.65 (m, 3H), 0.92 (s, 6H); MS (EI) for C₂₉H₃₇N₇O₃S: 564 (MH⁺).

2-amino-N-{[(3S)-1-methylpyrrolidin-3-yl]methyl}-5-[4-(6,6,7-trimethyl-5,6-dihydroquinazolin-4-yl)-2,3,4,5-tetrahydro-1,4-benzoxazepin-7-yl]pyridine-3-sulfonamide. Prepared according to the method of 5 by using 2-amino-5-bromo-N-{[(3S)-1-methylpyrrolidin-3-yl]methyl}pyridine-3-sulfonamide (reagent preparation 25) and [4-(6,6,7-trimethyl-5,6-dihydroquinazolin-4-yl)-2,3,4,5-tetrahydro-1,4-benzoxazepin-7-yl]boronic acid (reagent preparation 23) in step 1. ¹H NMR (400 MHz, Methanol-d₄): 8.35 (s, 1H), 8.25 (s, 1H), 8.07 (s, 1H), 7.37 (s, 1H), 7.32 (d, 1H), 6.97 (d, 1H), 6.07 (s, 1H), 4.58 (s, 2H), 4.23 (m, 2H), 3.84 (m, 2H), 2.86 to 2.78 (m, 3H), 2.72 (m, 2H), 2.63 (s, 2H), 2.46 (m, 1H), 2.40 (s, 3H), 2.34 (m, 1H), 1.93 (m, 1H), 1.83 (s, 3H), 1.46 (m, 1H), 0.99 (s, 6H); MS (EI) for C₃₁H₃₉N₇O₃S: 590 (MH⁺).

4-({2-amino-5-[4-(6,6-dimethyl-5,6,7,8-tetrahydroquinazolin-4-yl)-2,3,4,5-tetrahydro-1,4-benzoxazepin-7-yl]pyridin-3-yl}sulfonyl)-2-methylbutan-2-ol. Prepared according to the method of example 5 by using 4-[(2-amino-5-bromopyridin-3-yl)sulfonyl]-2-methylbutan-2-ol (reagent preparation 41) and [4-(6,6-dimethyl-5,6,7,8-tetrahydroquinazolin-4-yl)-2,3,4,5-tetrahydro-1,4-benzoxazepin-7-yl]boronic acid (reagent preparation 23) in step 1. ¹H NMR (400 MHz, Methanol-d₄): 8.52 (s, 1H), 8.35 (s, 1H), 8.14 (s, 1H), 7.50 (s, 1H), 7.43 (d, 1H), 7.06 (d, 1H), 4.70 (s, 2H), 4.30 (m, 2H), 3.95 (m, 2H), 3.35 (m, 2H), 2.79 (t, 2H), 2.48 (s, 2H), 1.82 (m, 2H), 1.68 (t, 2H), 1.15 (s, 6H), 0.90 (s, 6H); MS (EI) for C₂₉H₃₇N₅O₄S: 552 (MH⁺).

4-({2-amino-5-[4-(6,6-dimethyl-5,6,7,8-tetrahydroquinazolin-4-yl)-2,3,4,5-tetrahydro-1,4-benzoxazepin-7-yl]pyridin-3-yl}sulfinyl)-2-methylbutan-2-ol. Prepared according to the method of example 5 by using 4-[(2-amino-5-bromopyridin-3-yl)sulfinyl]-2-methylbutan-2-ol (reagent preparation 41) and [4-(6,6-dimethyl-5,6,7,8-tetrahydroquinazolin-4-yl)-2,3,4,5-tetrahydro-1,4-benzoxazepin-7-yl]boronic acid (reagent preparation 23) in step 1. ¹H NMR (400 MHz, Methanol-d₄): 8.40 (s, 1H), 8.34 (s, 1H), 8.03 (s, 1H), 7.51 (s, 1H), 7.42 (d, 1H), 7.05 (d, 1H), 4.68 (s, 2H), 4.33 (m, 2H), 3.94 (m, 2H), 3.20 (m, 2H), 2.79 (t, 2H), 2.47 (s, 2H), 1.80 (m, 2H), 1.67 (t, 2H), 1.21 (s, 6H), 0.88 (s, 6H); MS (EI) for C₂₉H₃₇N₅O₃S: 536 (MH⁺).

N-{5-[4-(6,6-dimethyl-5,6,7,8-tetrahydroquinazolin-4-yl)-2,3,4,5-tetrahydro-1,4-benzoxazepin-7-yl]-2-[(phenylmethyl)amino]pyridin-3-yl}methanesulfonamide. Prepared according to the method of example 5 by using N-{5-bromo-2-[(phenylmethyl)amino]pyridin-3-yl}methanesulfonamide (reagent preparation 39) and [4-(6,6-dimethyl-5,6,7,8-tetrahydroquinazolin-4-yl)-2,3,4,5-tetrahydro-1,4-benzoxazepin-7-yl]boronic acid (reagent preparation 23) in step 1. ¹H NMR (400 MHz, Methanol-D₄): 8.26 (s, 1H), 8.06 (s, 1H), 7.64 (s, 1H), 7.37 to 7.08 (m, 7H), 6.94 (d, 1H), 4.59 (s, 2H), 4.57 (s, 2H), 4.20 (m, 2H), 3.85 (m, 2H), 2.91 (s, 3H), 2.67 (t, 2H), 2.36 (s, 1.59 (t, 2H), 1.21 (s, 6H), 0.82 (s, 6H); MS (EI) for C₃₂H₃₆N₆O₃S: 585 (MH⁺).

N-{5-[4-(6,6-dimethyl-5,6,7,8-tetrahydroquinazolin-4-yl)-2,3,4,5-tetrahydro-1,4-benzoxazepin-7-yl]-2-(methylamino)pyridin-3-yl}methanesulfonamide. Prepared according to the method of example 5 by using N-[5-bromo-2-(methylamino)pyridin-3-yl]methanesulfonamide (reagent preparation 39) and [4-(6,6-dimethyl-5,6,7,8-tetrahydroquinazolin-4-yl)-2,3,4,5-tetrahydro-1,4-benzoxazepin-7-yl]boronic acid (reagent preparation 23) in step 1. ¹H NMR (400 MHz, Methanol-d₄): 8.35 (s, 1H), 8.13 (s, 1H), 7.71 (s, 1H), 7.44 (s, 1H), 7.39 (d, 1H), 7.03 (d, 1H), 4.68 (s, 2H), 4.32 (m, 2H), 3.94 (m, 2H), 3.00 (s, 3H), 2.98 (s, 3H), 2.79 (t, 2H), 2.46 (s, 2H), 1.67 (t, 2H), 0.90 (s, 6H); MS (EI) for C₂₆H₃₂N₆O₃S: 509 (MH⁺).

(2S)-3-[(2-amino-5-{4-[7-(methyloxy)quinazolin-4-yl]-2,3,4,5-tetrahydro-1,4-benzoxazepin-7-yl}pyridin-3-yl)sulfinyl]-2-methylpropan-1-ol. Prepared according to the method of example 5 by using (2S)-3-[(2-amino-5-bromopyridin-3-yl)sulfinyl]-2-methylpropan-1-ol (reagent preparation 41) and {4-[7-(methyloxy)quinazolin-4-yl]-2,3,4,5-tetrahydro-1,4-benzoxazepin-7-yl}boronic acid (reagent preparation 23) in step 1. ¹H NMR (400 MHz, Methanol-d₄): 8.42 (s, 1H), 8.36 (br, 1H), 8.02 (br, 1H), 7.93 (d, 1H), 7.52 (br, 1H), 7.40 (d, 1H), 7.08 (m, 2H), 7.01 (d, 1H), 5.03 (s, 2H), 4.43 (m, 2H), 4.22 (m, 2H), 3.90 (s, 3H), 3.64 (dd, 0.5H), 3.57 to 3.46 (m, 1.5H), 3.40 (dd, 0.5H), 3.14 (dd, 0.5H), 3.01 (dd, 0.5H), 2.82 (dd, 0.5H), 2.15 (m, 1H), 1.12 (dd, 3H); MS (EI) for C₂₇H₂₉N₅O₄S: 520 (MH⁺).

(2S)-3-[(2-amino-5-{4-[7-(methyloxy)quinazolin-4-yl]-2,3,4,5-tetrahydro-1,4-benzoxazepin-7-yl}pyridin-3-yl)sulfonyl]-2-methylpropan-1-ol. Prepared according to the method of example 5 by using (2S)-3-[(2-amino-5-bromopyridin-3-yl)sulfonyl]-2-methylpropan-1-ol (reagent preparation 41) and {4-[7-(methyloxy)quinazolin-4-yl]-2,3,4,5-tetrahydro-1,4-benzoxazepin-7-yl}boronic acid (reagent preparation 23) in step 1. ¹H NMR (400 MHz, Methanol-d₄): 8.42 (s, 1H), 8.34 (br, 1H), 8.07 (br, 1H), 7.89 (d, 1H), 7.44 (s, 1H), 7.35 (d, 1H), 7.08 (m, 2H), 6.94 (d, 1H), 4.96 (s, 2H), 4.34 (m, 2H), 4.14 (m, 2H), 3.84 (s, 3H), 3.41 (m, 2H), 3.31 (dd, 1H), 2.98 (dd, 1H), 2.12 (m, 1H), 0.99 (d, 3H); MS (EI) for C₂₇H₂₉N₅O₅S: 536 (MH⁺).

4-(6,6-dimethyl-5,6,7,8-tetrahydroquinazolin-4-yl)-7-[4-(1H-imidazol-2-yl)phenyl]-2,3,4,5-tetrahydro-1,4-benzoxazepine. Prepared according to the method of example 5 by using 2-methylpropyl 2-(4-bromophenyl)-1H-imidazole-1-carboxylate (reagent preparation 11) in step 1 followed by isobutylcarbamate deprotection. ¹H NMR (400 MHz, DMSO-d₆): 12.54 (s, 1H), 8.38 (s, 1H), 8.00 (d, 2H), 7.74 (d, 2H), 7.72 (m, 1H), 7.57 (m, 1H), 7.27 (m, 1H), 7.04 (m, 2H), 4.65 (s, 2H), 4.34 (m, 2H), 3.84 (m, 2H), 2.71 (t, 2H), 2.47 (s, 2H), 1.61 (t, 2H), 0.85 (s, 6H); MS (EI) for C₂₈H₂₉N₅O: 452 (MH⁺).

N-ethyl-6-[4-(2,6,6-trimethyl-5,6,7,8-tetrahydroquinazolin-4-yl)-2,3,4,5-tetrahydro-1,4-benzoxazepin-7-yl]-1H-imidazo[4,5-b]pyridin-2-amine. Prepared according to the method of example 5 by using 6-bromo-N-ethyl-1-[(methyloxy)methyl]-1H-imidazo[4,5-b]pyridin-2-amine (reagent preparation 36) and [4-(2,6,6-trimethyl-5,6,7,8-tetrahydroquinazolin-4-yl)-2,3,4,5-tetrahydro-1,4-benzoxazepin-7-yl]boronic acid (reagent preparation 23) in step 1 followed by MOM deprotection. ¹H NMR (400 MHz, methanol-d₄): 8.15 (d, 1H), 7.64 (d, 1H), 7.55 (d, 1H), 7.43 (dd, 1H), 7.06 (d, 1H), 4.71 (s, 2H), 4.31 (m, 2H), 3.99 (m, 2H), 3.46 (q, 2H), 2.75 (t, 2H), 2.47 (s, 2H), 2.41 (s, 3H), 1.66 (t, 2H), 1.30 (t, 3H), 0.91 (s, 6H); MS (EI) for C₂₇H₃₀N₆O: 455 (MH⁺).

7-(2-methyl-1H-imidazo[4,5-b]pyridin-6-yl)-4-(2,6,6-trimethyl-5,6,7,8-tetrahydroquinazolin-4-yl)-2,3,4,5-tetrahydro-1,4-benzoxazepine. Prepared as acetate salt according to the method of example 5 by using 6-bromo-2-methyl-1-({[2-(trimethylsilyl)ethyl]oxy}methyl)-1H-imidazo[4,5-b]pyridine (reagent preparation 35) and [4-(2,6,6-trimethyl-5,6,7,8-tetrahydroquinazolin-4-yl)-2,3,4,5-tetrahydro-1,4-benzoxazepin-7-yl]boronic acid (reagent preparation 23) in step 1 followed by SEM deprotection. ¹H NMR (400 MHz, methanol-d₄): 8.52 (d, 1H), 8.05 (d, 1H), 7.63 (d, 1H), 7.50 (dd, 1H), 7.09 (d, 1H), 4.73 (s, 2H), 4.33 (m, 2H), 4.00 (m, 2H), 2.75 (t, 2H), 2.64 (s, 3H), 2.47 (s, 2H), 2.41 (s, 3H), 1.96 (s, 3H), 1.66 (t, 2H), 0.91 (s, 6H); MS (EI) for C₂₇H₃₀N₆O: 455 (MH⁺).

7-(1H-benzimidazol-6-yl)-4-(6,6-dimethyl-5,6,7,8-tetrahydroquinazolin-4-yl)-2,3,4,5-tetrahydro-1,4-benzoxazepine. Prepared according to the method of example 5 by using isobutyl 6-bromo-1H-benzo[d]imidazole-1-carboxylate (reagent preparation 11) in step 1. ¹H NMR (400 MHz, methanol-d₄): 8.34 (s, 1H), 8.18 (s, 1H), 7.78 (s, 1H), 7.64 (d, 1H), 7.56-7.46 (m, 3H), 7.04 (d, 1H), 4.67 (s, 2H), 4.33 (m, 2H), 3.94 (m, 2H), 2.78 (t, 2H), 2.48 (s, 2H), 1.66 (t, 2H), 0.89 (s, 6H); MS (EI) for C₂₆H₂₇N₅O: 426 (MH⁺).

3-{[(3S)-3-aminopyrrolidin-1-yl]sulfonyl}-5-[4-(6,6-dimethyl-5,6,7,8-tetrahydroquinazolin-4-yl)-2,3,4,5-tetrahydro-1,4-benzoxazepin-7-yl]pyridin-2-amine. Prepared as acetate salt according to the method of example 5 by using (S)-tert-butyl 1-(2-amino-5-bromopyridin-3-ylsulfonyl)pyrrolidin-3-ylcarbamate (reagent preparation 25) in step 1 followed by Boc deprotection. ¹H NMR (400 MHz, methanol-d₄): 8.48 (d, 1H), 8.33 (s, 1H), 8.10 (d, 1H), 7.48 (d, 1H), 7.41 (dd, 1H), 7.05 (d, 1H), 4.70 (s, 2H), 4.34 (m, 2H), 3.96 (m, 2H), 3.73 (m, 1H), 3.55 (m, 2H), 3.35 (m, 1H), 2.79 (t, 2H), 2.49 (s, 2H), 2.25 (m, 1H), 1.93 (s, 3H), 1.88 (m, 2H), 1.69 (t, 2H), 0.91 (s, 6H); MS (EI) for C₂₈H₃₅N₇O₃S: 550 (MH⁺).

3-{[(3R)-3-aminopyrrolidin-1-yl]sulfonyl}-5-[4-(6,6-dimethyl-5,6,7,8-tetrahydroquinazolin-4-yl)-2,3,4,5-tetrahydro-1,4-benzoxazepin-7-yl]pyridin-2-amine. Prepared as acetate salt according to the method of example 5 by using (R)-tert-butyl 1-(2-amino-5-bromopyridin-3-ylsulfonyl)pyrolidin-3-ylcarbamate (reagent preparation 25) in step 1 followed by Boc deprotection. ¹H NMR (400 MHz, methanol-d₄): 8.48 (d, 1H), 8.33 (s, 1H), 8.10 (d, 1H), 7.48 (d, 1H), 7.41 (dd, 1H), 7.05 (d, 1H), 4.70 (s, 2H), 4.34 (m, 2H), 3.96 (m, 2H), 3.66 (m, 1H), 3.55 (m, 2H), 3.35 (m, 1H), 2.79 (t, 2H), 2.49 (s, 2H), 2.25 (m, 1H), 1.93 (s, 3H), 1.88 (m, 2H), 1.69 (t, 2H), 0.91 (s, 6H); MS (EI) for C₂₈H₃₅N₇O₃S: 550 (MH⁺).

8-({2-amino-5-[4-(6,6-dimethyl-5,6,7,8-tetrahydroquinazolin-4-yl)-2,3,4,5-tetrahydro-1,4-benzoxazepin-7-yl]pyridin-3-yl}sulfonyl)-8-azabicyclo[3.2.1]octan-3-amine. Prepared according to the method of example 5 by using tert-butyl 8-(2-amino-5-bromopyridin-3-ylsulfonyl)-8-azabicyclo[3.2.1]octan-3-ylcarbamate (reagent preparation 25) in step 1 followed by Boc deprotection. MS (EI) for C₃₁H₃₉H₇O₃S: 590 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-[(3R)-pyrrolidin-3-ylmethyl]pyridine-3-sulfonamide. Prepared as acetate salt according to the method of example 5 by using (R)-tert-butyl 3-((2-amino-5-bromopyridine-3-sulfonamido)methyl)pyrrolidine-1-carboxylate (reagent preparation 25) in step 1 followed by Boc deprotection. ¹H NMR (400 MHz, methanol-d₄): 8.45 (d, 1H), 8.33 (s, 1H), 8.16 (d, 1H), 7.50 (d, 1H), 7.41 (dd, 1H), 7.06 (d, 1H), 4.71 (s, 2H), 4.34 (m, 2H), 3.96 (m, 2H), 3.15 (m, 1H), 2.98-2.89 (m, 3H), 2.79 (t, 2H), 2.48 (s, 2H), 2.09 (m, 1H), 1.90 (s, 3H), 1.69 (t, 2H), 0.92 (s, 6H); MS (EI) for C₂₉H₃₇N₇O₃S: 564 (MH⁺).

2-amino-N-8-azabicyclo[3.2.1]oct-3-yl-5-[4-(6,6-dimethyl-5,6,7,8-tetrahydroquinazolin-4-yl)-2,3,4,5-tetrahydro-1,4-benzoxazepin-7-yl]pyridine-3-sulfonamide. Prepared as acetate salt according to the method of example 5 by using 2,2,2-trichloroethyl 3-(2-amino-5-bromopyridine-3-sulfonamido)-8-azabicyclo[3.2.1]octane-8-carboxylate (reagent preparation 25) in step 1 followed by Troc deprotection. ¹H NMR (400 MHz, methanol-d₄): 8.53 (s, 1H), 8.49 (d, 1H), 8.45 (s, 1H), 7.67 (d, 1H), 7.48 (dd, 1H), 7.06 (d, 1H), 5.15 (s, 2H), 4.47 (m, 2H), 4.31 (m, 2H), 4.01 (m, 2H), 3.44 (m, 1H), 2.86 (t, 2H), 2.60 (s, 2H), 2.46 (m, 2H), 2.26-2.08 (m, 6H), 1.71 (t, 2H), 0.96 (s, 6H); MS (EI) for C₃₁H₃₉N₇O₃S: 590 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-[(3S)-pyrrolidin-3-ylmethyl]pyridine-3-sulfonamide. Prepared according to the method of example 5 by using (S)-tert-butyl 3-((2-amino-5-bromopyridine-3-sulfonamido)methyl)pyrrolidine-1-carboxylate (reagent preparation 25) in step 1 followed by Boc deprotection. ¹H NMR (400 MHz, methanol-d₄): 8.45 (d, 1H), 8.33 (s, 1H), 8.16 (d, 1H), 7.49 (d, 1H), 7.41 (dd, 1H), 7.05 (d, 1H), 4.71 (s, 2H), 4.34 (m, 2H), 3.96 (m, 2H), 3.26-3.06 (m, 3H), 2.94 (d, 2H), 2.85 (m, 1H), 2.80 (t, 2H), 2.48 (s, 2H), 2.43 (m, 1H), 2.04 (m, 1H), 1.69 (t, 2H), 1.62 (m, 1H), 0.92 (s, 6H); MS (EI) for C₂₉H₃₇N₇O₃S: 564 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-[(3R)-pyrrolidin-3-yl]pyridine-3-sulfonamide. Prepared as acetate salt according to the method of example 5 by using (R)-tert-butyl 3-(2-amino-5-bromopyridine-3-sulfonamido)pyrrolidine-1-carboxylate (reagent preparation 25) in step 1 followed by Boc deprotection. ¹H NMR (400 MHz, methanol-d₄): 8.48 (d, 1H), 8.33 (s, 1H), 8.18 (d, 1H), 7.50 (d, 1H), 7.41 (dd, 1H), 7.05 (d, 1H), 4.70 (s, 2H), 4.34 (m, 2H), 3.96 (m, 2H), 3.90 (m, 1H), 3.20 (m, 3H), 2.80 (t, 2H), 2.49 (s, 2H), 2.11 (m, 1H), 1.92 (s, 3H), 1.88 (m, 2H), 1.68 (t, 2H), 0.91 (s, 6H); MS (EI) for C₂₈H₃₅N₇O₃S: 550 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-[(3S)-pyrrolidin-3-yl]pyridine-3-sulfonamide. Prepared as acetate salt according to the method of example 5 by using (S)-tert-butyl 3-(2-amino-5-bromopyridine-3-sulfonamido)pyrrolidine-1-carboxylate (reagent preparation 25) in step 1 followed by Boc deprotection. ¹H NMR (400 MHz, methanol-d₄): 8.48 (d, 1H), 8.33 (s, 1H), 8.18 (d, 1H), 7.50 (d, 1H), 7.41 (dd, 1H), 7.05 (d, 1H), 4.70 (s, 2H), 4.34 (m, 2H), 3.96 (m, 2H), 3.90 (m, 1H), 3.20 (m, 3H), 2.80 (t, 2H), 2.49 (s, 2H), 2.11 (m, 1H), 1.92 (s, 3H), 1.88 (m, 2H), 1.68 (t, 2H), 0.91 (s, 6H); MS (EI) for C₂₈H₃₅N₇O₃S: 550 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-(piperidin-3-ylmethyl)pyridine-3-sulfonamide. Prepared as acetate salt according to the method of example 5 by using tert-butyl 3-((2-amino-5-bromopyridine-3-sulfonamido)methyl)piperidine-1-carboxylate (reagent preparation 25) in step 1 followed by Boc deprotection. ¹H NMR (400 MHz, methanol-d₄): 8.45 (d, 1H), 8.33 (s, 1H), 8.15 (d, 1H), 7.50 (d, 1H), 7.41 (dd, 1H), 7.06 (d, 1H), 4.71 (s, 2H), 4.33 (m, 2H), 3.96 (m, 2H), 2.81 (m, 5H), 2.48 (s, 2H), 1.90 (s, 3H), 1.86 (m, 2H), 1.69 (t, 2H), 0.91 (s, 6H); MS (EI) for C₃₀H₃₉N₇O₃S: 578 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-(piperidin-2-ylmethyl)pyridine-3-sulfonamide. Prepared as dihydrochloride salt according to the method of example 5 by using tert-butyl 2-((2-amino-5-bromopyridine-3-sulfonamido)methyl)piperidine-1-carboxylate (reagent preparation 25) in step 1 followed by Boc deprotection. MS (EI) for C₃₀H₃₉N₇O₃S: 578 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-[(3R)-piperidin-3-ylmethyl]pyridine-3-sulfonamide. Prepared as acetate salt according to the method of example 5 by using (R)-tert-butyl 3-((2-amino-5-bromopyridine-3-sulfonamido)methyl)piperidine-1-carboxylate (reagent preparation 25) in step 1 followed by Boc deprotection. ¹H NMR (400 MHz, methanol-d₄); 8.45 (d, 1H), 8.33 (s, 1H), 8.15 (d, 1H), 7.49 (d, 1H), 7.41 (dd, 1H), 7.05 (d, 1H), 4.71 (s, 2H), 4.33 (m, 2H), 3.96 (m, 2H), 2.87-2.76 (m, 6H), 2.64 (m, 1H), 2.49 (s, 2H), 1.91 (s, 3H), 1.87 (m, 2H), 1.71-1.57 (m, 3H), 1.22 (m, 1H), 0.91 (s, 6H); MS (EI) for C₃₀H₃₉N₇O₃S: 578 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-[(3S)-piperidin-3-ylmethyl]pyridine-3-sulfonamide. Prepared as acetate salt according to the method of example 5 by using (S)-tert-butyl 3-((2-amino-5-bromopyridine-3-sulfonamido)methyl)piperidine-1-carboxylate (reagent preparation 25) in step 1 followed by Boc deprotection. ¹H NMR (400 MHz, methanol-d₄): 8.45 (d, 1H), 8.33 (s, 1H), 8.15 (d, 1H), 7.49 (d, 1H), 7.41 (dd, 1H), 7.05 (d, 1H), 4.71 (s, 2H), 4.33 (m, 2H), 3.96 (m, 2H), 2.87-2.76 (m, 6H), 2.64 (m, 1H), 2.49 (s, 2H), 1.91 (s, 3H), 1.87 (m, 2H), 1.71-1.57 (m, 3H), 1.22 (m, 1H), 0.91 (s, 6H); MS (EI) for C₃₀H₃₉N₇O₃S: 578 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-{[(3S)-1-methylpiperidin-3-yl]methyl}pyridine-3-sulfonamide. Prepared as acetate salt according to the method of example 5 by using (S)-2-amino-5-bromo-N-((1-methylpiperidin-3-yl)methyl)pyridine-3-sulfonamide (reagent preparation 25) in step 1. ¹H NMR (400 MHz, methanol-d₄): 8.44 (d, 1H), 8.34 (s, 1H), 8.15 (d, 1H), 7.49 (d, 1H), 7.41 (dd, 1H), 7.06 (d, 1H), 4.71 (s, 2H), 4.34 (m, 2H), 3.96 (m, 2H), 3.08 (m, 1H), 2.80 (m, 4H), 2.48 (s, 5H), 2.31 (m, 1H), 2.06 (m, 1H), 1.91 (s, 3H), 1.77 (m, 3H), 1.69 (t, 2H), 1.60 (m, 1H), 1.01 (m, 1H), 0.91 (s, 6H); MS (EI) for C₃₁H₄₁N₇O₃S: 592 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N,N-dimethylpyridine-3-sulfonamide. Prepared according to the method of example 5 by using 2-amino-5-bromo-N,N-dimethylpyridine-3-sulfonamide (reagent preparation 25) in step 1. ¹H NMR (400 MHz, methanol-d₄): 8.47 (s, 1H), 8.35 (s, 1H), 8.05 (s, 1H), 7.49 (s, 1H), 7.42 (d, 1H), 7.07 (d, 1H), 4.71 (s, 2H), 4.35 (m, 2H), 3.97 (m, 2H), 2.81 (m, 8H), 2.49 (s, 2H), 1.70 (m, 2H), 0.91 (s, 6H); MS (EI) for C₂₆H₃₂N₆O₃S: 509 (MH⁺).

5-[4-(6,6-dimethyl-5,6,7,8-tetrahydroquinazolin-4-yl)-2,3,4,5-tetrahydro-1,4-benzoxazepin-7-yl]pyridine-3-sulfonamide. Prepared as acetate salt according to the method of example 5 by using 5-bromopyridine-3-sulfonamide in step 1. ¹H NMR (400 MHz, methanol-d₄): 9.00 (s, 1H), 8.96 (s, 1H), 8.46 (s, 1H), 8.34 (s, 1H), 7.69 (s, 1H), 7.58 (d, 1H), 7.13 (d, 1H), 4.77 (s, 2H), 4.38 (m, 2H), 3.98 (m, 2H), 2.79 (m, 2H), 2.48 (s, 2H), 1.98 (s, 3H), 1.68 (m, 2H), 0.89 (s, 6H); MS (EI) for C₂₄H₂₇N₅O₃S: 466 (MH⁺).

2-amino-N,N-dimethyl-5-[4-(2,6,6-trimethyl-5,6,7,8-tetrahydroquinazolin-4-yl)-2,3,4,5-tetrahydro-1,4-benzoxazepin-7-yl]pyridine-3-sulfonamide. Prepared according to the method of example 5 by using 4-(2,6,6-trimethyl-5,6,7,8-tetrahydroquinazolin-4-yl)-2,3,4,5-tetrahydrobenzo[f][1,4]oxazepin-7-ylboronic acid (reagent preparation 23) and 2-amino-5-bromo-N,N-dimethylpyridine-3-sulfonamide (reagent preparation 25) in step 1. ¹H NMR (400 MHz, methanol-d₄): 8.47 (s, 1H), 8.04 (s, 1H), 7.52 (s, 1H), 7.41 (d, 1H), 7.06 (d, 1H), 4.71 (s, 2H), 4.31 (m, 2H), 4.00 (m, 2H), 2.80 (s, 6H), 2.75 (m, 2H), 2.45 (s, 2H), 2.40 (s, 3H), 1.67 (m, 2H), 0.91 (s, 6H); MS (EI) for C₂₇H₃₄N₆O₃S: 523 (MH⁺).

5-[4-(6,6-dimethyl-5,6,7,8-tetrahydroquinazolin-4-yl)-2,3,4,5-tetrahydro-1,4-benzoxazepin-7-yl]-N,N-dimethylpyridine-3-sulfonamide. Prepared according to the method of example 5 by using 5-bromo-N,N-dimethylpyridine-3-sulfonamide in step 1. ¹H NMR (400 MHz, methanol-d₄): 9.08 (s, 1H), 8.88 (s, 1H), 8.33 (m, 2H), 7.60 (d, 1H), 7.14 (d, 1H), 4.77 (s, 2H), 4.40 (m, 2H), 3.99 (m, 2H), 2.79 (m, 8H), 2.49 (s, 2H), 1.69 (m, 2H), 0.90 (s, 6H); MS (EI) for C₂₆H₃₁N₅O₃S: 494 (MH⁺).

5-[4-(6,6-dimethyl-5,6,7,8-tetrahydroquinazolin-4-yl)-2,3,4,5-tetrahydro-1,4-benzoxazepin-7-yl]-3-(morpholin-4-ylsulfonyl)pyridin-2-amine. Prepared as acetate salt according to the method of example 5 by using 5-bromo-3-(morpholinosulfonyl)pyridin-2-amine (reagent preparation 25) in step 1. ¹H NMR (400 MHz, methanol-d₄): 8.48 (d, 1H), 8.35 (s, 1H), 8.03 (d, 1H), 7.49 (d, 1H), 7.42 (dd, 1H), 7.06 (d, 1H), 4.71 (s, 2H), 4.35 (m, 2H), 3.97 (m, 2H), 3.70 (m, 4H), 3.13 (m, 4H), 2.80 (t, 2H), 2.48 (s, 2H), 1.98 (s, 3H), 1.61 (t, 2H), 0.90 (s, 6H); MS (EI) for C₂₈H₃₄N₆O₄S: 551 (MH⁺).

5-[4-(6,6-dimethyl-5,6,7,8-tetrahydroquinazolin-4-yl)-2,3,4,5-tetrahydro-1,4-benzoxazepin-7-yl]-3-[(4-methylpiperazin-1-yl)sulfonyl]pyridin-2-amine. Prepared as diacetate salt according to the method of example 5 by using 5-bromo-3-(4-methylpiperazin-1-ylsulfonyl)pyridin-2-amine (reagent preparation 25) in step 1. ¹H NMR (400 MHz, methanol-d₄): 8.58 (d, 1H), 8.34 (s, 1H), 8.04 (d, 1H), 7.49 (d, 1H), 7.41 (dd, 1H), 7.06 (d, 1H), 4.71 (s, 2H), 4.35 (m, 2H), 3.96 (m, 2H), 3.22 (m, 4H), 2.80 (t, 2H), 2.55 (m, 4H), 2.49 (s, 2H), 2.31 (s, 3H), 1.97 (s, 3H), 1.69 (t, 2H), 0.90 (s, 6H); MS (EI) for C₂₉H₃₇N₇O₃S: 564 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N,N-dimethylpyridine-3-carboxamide. Prepared as trifluoroacetate salt according to the method of example 5 by using 2-amino-5-bromo-N,N-dimethyl-nicotinamide (reagent preparation 33) in step 1. ¹H NMR (400 MHz, methanol-d₄): 8.51 (d, 1H), 8.23 (s, 2H), 7.66 (d, 1H), 7.50 (dd, 1H), 7.05 (d, 1H), 5.14 (s, 2H), 4.48 (m, 2H), 4.32 (m, 2H), 3.14 (s, 3H), 3.07 (s, 3H), 2.85 (t, 2H), 2.59 (s, 2H), 1.70 (t, 2H), 0.95 (s, 6H); MS (EI) for C₂₇H₃₂N₆O₂: 473 (MH⁺).

3-(azetidin-1-ylsulfonyl)-5-[4-(6,6-dimethyl-5,6,7,8-tetrahydroquinazolin-4-yl)-2,3,4,5-tetrahydro-1,4-benzoxazepin-7-yl]pyridin-2-amine. Prepared as acetate salt according to the method of example 5 by using 3-(azetidin-1-ylsulfonyl)-5-bromopyridin-2-amine (reagent preparation 25) in step 1. ¹H NMR (400 MHz, methanol-d₄): 8.51 (d, 1H), 8.34 (s, 1H), 8.09 (d, 1H), 7.50 (d, 1H), 7.43 (dd, 1H), 7.07 (d, 1H), 4.71 (s, 2H), 4.35 (m, 2H), 3.96 (m, 2H), 3.88 (t, 4H), 2.79 (t, 2H), 2.49 (s, 2H), 2.15 (m, 2H), 1.96 (s, 3H), 1.69 (t, 2H), 0.90 (s, 6H); MS (EI) for C₂₇H₃₂N₆O₃S: 521 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-methylpyridine-3-sulfonamide. Prepared according to the method of example 5 by using 2-amino-5-bromo-N-methylpyridine-3-sulfonamide (reagent preparation 25) in step 1. ¹H NMR (400 MHz, methanol-d₄): 8.44 (d, 1H), 8.34 (s, 1H), 8.15 (d, 1H), 7.49 (d, 1H), 7.42 (dd, 1H), 7.06 (d, 1H), 4.70 (s, 2H), 4.35 (m, 2H), 3.96 (m, 2H), 2.79 (t, 2H), 2.55 (s, 3H), 2.48 (s, 2H), 1.69 (t, 2H), 0.90 (s, 6H); MS (EI) for C₂₅H₃₀N₆O₃S: 495 (MH⁺).

1-({2-amino-5-[4-(6,6-dimethyl-5,6,7,8-tetrahydroquinazolin-4-yl)-2,3,4,5-tetrahydro-1,4-benzoxazepin-7-yl]pyridin-3-yl}sulfonyl)azetidin-3-ol. Prepared according to the method of example 5 by using 1-(2-amino-5-bromopyridin-3-ylsulfonyl)azetidin-3-ol (reagent preparation 25) in step 1. ¹H NMR (400 MHz, methanol-d₄): 8.51 (d, 1H), 8.34 (s, 1H), 8.09 (d, 1H), 7.50 (d, 1H), 7.43 (dd, 1H), 7.07 (d, 1H), 4.70 (s, 2H), 4.45 (m, 2H), 4.35 (m, 2H), 4.02 (m, 2H), 3.96 (m, 2H), 3.65 (m, 2H), 2.80 (t, 2H), 2.49 (s, 2H), 1.69 (t, 2H), 0.91 (s, 6H); MS (EI) for C₂₇H₃₂N₆O₄S: 537 (MH⁺).

5-[4-(6,6-dimethyl-5,6,7,8-tetrahydroquinazolin-4-yl)-2,3,4,5-tetrahydro-1,4-benzoxazepin-7-yl]-3-(pyrrolidin-1-ylsulfonyl)pyridin-2-amine. Prepared as acetate salt according to the method of example 5 by using 5-bromo-3-(pyrrolidin-1-ylsulfonyl)pyridin-2-amine (reagent preparation 25) in step 1. ¹H NMR (400 MHz, methanol-d₄): 8.45 (d, 1H), 8.34 (s, 1H), 8.10 (d, 1H), 7.48 (d, 1H), 7.41 (dd, 1H), 7.06 (d, 1H), 4.70 (s, 2H), 4.35 (m, 2H), 3.96 (m, 2H), 3.32 (m, 4H), 2.80 (t, 2H), 2.49 (s, 2H), 1.96 (s, 3H), 1.84 (m, 4H), 1.69 (t, 2H), 0.91 (s, 6H); MS (EI) for C₂₈H₃₄N₆O₃S: 535 (MH⁺).

1-({2-amino-5-[4-(6,6-dimethyl-5,6,7,8-tetrahydroquinazolin-4-yl)-2,3,4,5-tetrahydro-1,4-benzoxazepin-7-yl]pyridin-3-yl}sulfonyl)pyrrolidin-3-ol. Prepared according to the method of example 5 by using 1-(2-amino-5-bromopyridin-3-ylsulfonyl)pyrrolidin-3-ol (reagent preparation 25) in step 1. ¹H NMR (400 MHz, methanol-d₄): 8.44 (d, 1H), 8.34 (s, 1H), 8.11 (d, 1H), 7.48 (d, 1H), 7.41 (dd, 1H), 7.06 (d, 1H), 4.70 (s, 2H), 4.35 (m, 2H), 3.95 (m, 2H), 3.47 (m, 3H), 2.79 (t, 2H), 2.49 (s, 2H), 2.03-1.80 (m, 4H), 1.69 (t, 2H), 0.91 (s, 6H); MS (EI) for C₂₈H₃₄N₆O₄S: 551 (MH⁺).

2-amino-N-cyclobutyl-5-[4-(6,6-dimethyl-5,6,7,8-tetrahydroquinazolin-4-yl)-2,3,4,5-tetrahydro-1,4-benzoxazepin-7-yl]pyridine-3-sulfonamide. Prepared according to the method of example 5 by using 2-amino-5-bromo-N-cyclobutylpyridine-3-sulfonamide (reagent preparation 25) in step 1. ¹H NMR (400 MHz, methanol-d₄): 8.42 (d, 1H), 8.34 (s, 1H), 8.15 (d, 1H), 7.48 (d, 1H), 7.41 (dd, 1H), 7.06 (d, 1H), 4.70 (s, 2H), 4.35 (m, 2H), 3.95 (m, 2H), 3.72 (m, 1H), 2.80 (t, 2H), 2.48 (s, 2H), 2.02 (m, 2H), 1.82 (m, 2H), 1.68 (t, 2H), 1.57 (m, 2H), 0.90 (s, 6H); MS (EI) for C₂₈H₃₄N₆O₃S: 535 (MIT).

5-[4-(6,6-dimethyl-5,6,7,8-tetrahydroquinazolin-4-yl)-2,3,4,5-tetrahydro-1,4-benzoxazepin-7-yl]-3-(methylsulfonyl)pyridin-2-amine. Prepared as acetate salt according to the method of example 5 by using 5-bromo-3-(methylsulfonyl)pyridin-2-amine (reagent preparation 34) in step 1. ¹H NMR (400 MHz, methanol-d₄): 8.52 (d, 1H), 8.34 (s, 1H), 8.19 (d, 1H), 7.52 (d, 1H), 7.43 (dd, 1H), 7.06 (d, 1H), 4.71 (s, 2H), 4.34 (m, 2H), 3.96 (m, 2H), 3.15 (s, 3H), 2.79 (t, 2H), 2.49 (s, 2H), 1.97 (s, 3H), 1.69 (t, 2H), 0.91 (s, 6H); MS (EI) for C₂₅H₂₉N₅O₃S: 480 (MH⁺).

2-amino-5-[4-(6,6-dimethyl-5,6,7,8-tetrahydroquinazolin-4-yl)-2,3,4,5-tetrahydro-1,4-benzoxazepin-7-yl]pyridine-3-sulfonamide. Prepared according to the method of example 5 by using 2-amino-5-bromopyridine-3-sulfonamide (reagent preparation 25) in step 1. MS (EI) for C₂₄H₂₈N₆O₃S: 481 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-ethyl-N-methylpyridine-3-sulfonamide. Prepared according to the method of example 5 by using 2-amino-5-bromo-N-ethyl-N-methylpyridine-3-sulfonamide (reagent preparation 25) in step 1. ¹H NMR (400 MHz, methanol-d₄): 8.44 (d, 1H), 8.34 (s, 1H), 8.07 (d, 1H), 7.48 (d, 1H), 7.41 (dd, 1H), 7.06 (d, 1H), 4.70 (s, 2H), 4.35 (m, 2H), 3.96 (m, 2H), 3.24 (q, 2H), 2.83 (s, 3H), 2.80 (t, 2H), 2.48 (s, 2H), 1.69 (t, 2H), 1.14 (t, 3H), 0.90 (s, 6H); MS (EI) for C₂₇H₃₄N₆O₃S: 523 (MH⁺).

3-[(3,3-difluoroazetidin-1-yl)sulfonyl]-5-[4-(6,6-dimethyl-5,6,7,8-tetrahydroquinazolin-4-yl)-2,3,4,5-tetrahydro-1,4-benzoxazepin-7-yl]pyridin-2-amine. Prepared according to the method of example 5 by using 5-bromo-3-(3,3-difluoroazetidin-1-ylsulfonyl)pyridin-2-amine (reagent preparation 25) in step 1. ¹H NMR (400 MHz, methanol-d₄): 8.55 (d, 1H), 8.34 (s, 1H), 8.14 (d, 1H), 7.52 (d, 1H), 7.44 (dd, 1H), 7.07 (d, 1H), 4.70 (s, 2H), 4.32 (m, 6H), 3.96 (m, 2H), 2.79 (t, 2H), 2.49 (s, 2H), 1.69 (t, 2H), 0.90 (s, 6H); MS (EI) for C₂₇H₃₀F₂N₆O₃S: 523 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-(2-hydroxy-1-methylethyl)pyridine-3-sulfonamide. Prepared according to the method of example 5 by using 2-amino-5-bromo-N-(1-hydroxypropan-2-yl)pyridine-3-sulfonamide (reagent preparation 25) in step 1. ¹H NMR (400 MHz, methanol-d₄): 8.43 (d, 1H), 8.34 (s, 1H), 8.20 (d, 1H), 7.50 (d, 1H), 7.42 (dd, 1H), 7.06 (d, 1H), 4.70 (s, 2H), 4.35 (m, 2H), 3.95 (m, 2H), 3.44 (m, 1H), 3.30 (m, 2H), 2.80 (t, 2H), 2.48 (s, 2H), 1.69 (t, 2H), 1.03 (d, 3H), 0.90 (s, 6H); MS (EI) for C₂₇H₃₄N₆O₄S: 539 (MH⁺).

2-amino-5-[4-(6,6-dimethyl-5,6-dihydroquinazolin-4-yl)-2,3,4,5-tetrahydro-1,4-benzoxazepin-7-yl]-N-methylpyridine-3-sulfonamide. Prepared according to the method of example 5 by using 4-chloro-6,6-dimethyl-5,6-dihydroquinazoline (reagent preparation 23) and 2-amino-5-bromo-N-methylpyridine-3-sulfonamide (reagent preparation 25) in step 1. ¹H NMR (400 MHz, methanol-d₄): 8.43 (d, 1H), 8.38 (s, 1H), 8.15 (d, 1H), 7.48 (d, 1H), 7.42 (dd, 1H), 7.06 (d, 1H), 6.33 (d, 1H), 6.27 (d, 1H), 4.69 (s, 2H), 4.35 (m, 2H), 3.95 (m, 2H), 2.76 (s, 2H), 2.55 (s, 3H), 1.01 (s, 6H); MS (EI) for C₂₅H₂₈N₆O₃S: 493 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-(2-fluoroethyl)pyridine-3-sulfonamide. Prepared according to the method of example 5 by using 2-amino-5-bromo-N-(2-fluoroethyl)pyridine-3-sulfonamide (reagent preparation 25) in step 1. ¹H NMR (400 MHz, methanol-d₄): 8.43 (d, 1H), 8.34 (s, 1H), 8.17 (d, 1H), 7.49 (d, 1H), 7.41 (dd, 1H), 7.05 (d, 1H), 4.70 (s, 2H), 4.40 (m, 2H), 4.34 (m, 2H), 3.95 (m, 2H), 3.20 (m, 2H), 2.79 (t, 2H), 2.48 (s, 2H), 1.69 (t, 2H), 0.90 (s, 6H); MS (EI) for C₂₆H₃FN₆O₃S: 527 (MH⁺).

3-[(3-aminopyrrolidin-1-yl)sulfonyl]-5-[4-(6,6-dimethyl-5,6,7,8-tetrahydroquinazolin-4-yl)-2,3,4,5-tetrahydro-1,4-benzoxazepin-7-yl]pyridin-2-amine. Prepared as dihydrochloride salt according to the method of example 5 by using tert-butyl 1-(2-amino-5-bromopyridin-3-ylsulfonyl)pyrrolidin-3-ylcarbamate (reagent preparation 25) in step 1 followed by Boc deprotection. ¹H NMR (400 MHz, methanol-d₄): 8.51 (m, 2H), 7.70 (s, 1H), 7.51 (d, 1H), 7.07 (d, 1H), 5.17 (s, 2H), 4.48 (m, 2H), 4.31 (m, 2H), 3.95 (m, 1H), 3.69 (m, 2H), 3.57 (m, 1H), 3.45 (m, 1H), 2.86 (t, 2H), 2.61 (s, 2H), 2.41 (m, 1H), 2.06 (m, 1H), 1.71 (t, 2H), 0.96 (s, 6H); MS (EI) for C₂₈H₃₅N₇O₃S: 550 (MH⁺).

1-({2-amino-5-[4-(6,6-dimethyl-5,6,7,8-tetrahydroquinazolin-4-yl)-2,3,4,5-tetrahydro-1,4-benzoxazepin-7-yl]pyridin-3-yl}sulfonyl)piperidin-4-ol. Prepared according to the method of example 5 by using 1-(2-amino-5-bromopyridin-3-ylsulfonyl)piperidin-4-ol (reagent preparation 25) in step 1. MS (EI) for C₂₉H₃₆N₆O₄S: 565 (MH⁺).

3-[(3-aminopiperidin-1-yl)sulfonyl]-5-[4-(6,6-dimethyl-5,6,7,8-tetrahydroquinazolin-4-yl)-2,3,4,5-tetrahydro-1,4-benzoxazepin-7-yl]pyridin-2-amine. Prepared as acetate salt according to the method of example 5 by using tert-butyl 1-(2-amino-5-bromopyridin-3-ylsulfonyl)piperidin-3-ylcarbamate (reagent preparation 25) in step 1 followed by Boc deprotection. ¹H NMR (400 MHz, methanol-d₄): 8.49 (d, 1H), 8.33 (s, 1H), 8.04 (d, 1H), 7.48 (d, 1H), 7.40 (dd, 1H), 7.06 (d, 1H), 4.71 (s, 2H), 4.34 (m, 2H), 3.96 (m, 2H), 3.58 (m, 1H), 3.38 (m, 1H), 3.19 (m, 1H), 2.97 (m, 1H), 2.88 (m, 1H), 2.80 (t, 2H), 2.49 (s, 2H), 1.93 (s, 3H), 1.87 (m, 2H), 1.67 (m, 3H), 1.42 (m, 1H), 0.91 (s, 6H); MS (EI) for C₂₉H₃₇N₇O₃S: 564 (MH⁺).

2-amino-N-(2-aminoethyl)-5-[4-(6,6-dimethyl-5,6,7,8-tetrahydroquinazolin-4-yl)-2,3,4,5-tetrahydro-1,4-benzoxazepin-7-yl]pyridine-3-sulfonamide. Prepared as acetate salt according to the method of example 5 by using tert-butyl 2-(2-amino-5-bromopyridine-3-sulfonamido)ethylcarbamate (reagent preparation 25) in step 1 followed by Boc deprotection. ¹H NMR (400 MHz, methanol-d₄): 8.47 (d, 1H), 8.33 (s, 1H), 8.17 (d, 1H), 7.50 (d, 1H), 7.41 (dd, 1H), 7.06 (d, 1H), 4.72 (s, 2H), 4.34 (m, 2H), 3.96 (m, 2H), 3.08 (t, 2H), 2.97 (t, 2H), 2.78 (t, 2H), 2.48 (s, 2H), 1.92 (s, 3H), 1.68 (m, 3H), 0.91 (s, 6H); MS (EI) for C₂₆H₃₃N₇O₃S: 524 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-(3-hydroxypropyl)pyridine-3-sulfonamide. Prepared according to the method of example 5 by using 2-amino-5-bromo-N-(3-hydroxypropyl)pyridine-3-sulfonamide (reagent preparation 25) in step 1. ¹H NMR (400 MHz, methanol-d₄): 8.43 (d, 1H), 8.34 (s, 1H), 8.16 (d, 1H), 7.49 (d, 1H), 7.42 (dd, 1H), 7.05 (d, 1H), 4.70 (s, 2H), 4.35 (m, 2H), 3.95 (m, 2H), 3.55 (t, 2H), 2.98 (t, 2H), 2.80 (t, 2H), 2.48 (s, 2H), 1.67 (m, 4H), 0.90 (s, 6H); MS (EI) for C₂₇H₃₄N₆O₄S: 539 (MH⁺).

2-amino-N-(3-aminopropyl)-5-[4-(6,6-dimethyl-5,6,7,8-tetrahydroquinazolin-4-yl)-2,3,4,5-tetrahydro-1,4-benzoxazepin-7-yl]pyridine-3-sulfonamide. Prepared as dihydrochloride salt according to the method of example 5 by using tert-butyl 3-(2-amino-5-bromopyridine-3-sulfonamido)propylcarbamate (reagent preparation 25) in step 1 followed by Boc deprotection. ¹H NMR (400 MHz, methanol-d₄): 8.57 (s, 1H), 8.53 (s, 1H), 8.46 (s, 1H), 7.73 (s, 1H), 7.52 (d, 1H), 7.07 (d, 1H), 5.16 (s, 2H), 4.48 (m, 2H), 4.32 (m, 2H), 3.31 (t, 2H), 3.04 (t, 2H), 2.86 (t, 2H), 2.61 (s, 2H), 1.90 (m, 2H), 1.71 (t, 2H), 0.96 (s, 6H); MS (EI) for C₂₇H₃₅N₇O₃S: 538 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-(3,3,3-trifluoro-2-hydroxypropyl)pyridine-3-sulfonamide. Prepared according to the method of example 5 by using 2-amino-5-bromo-N-(3,3,3-trifluoro-2-hydroxypropyl)pyridine-3-sulfonamide (reagent preparation 25) in step 1. ¹H NMR (400 MHz, methanol-d₄): 8.44 (d, 1H), 8.34 (s, 1H), 8.18 (d, 1H), 7.48 (d, 1H), 7.42 (dd, 1H), 7.06 (d, 1H), 4.69 (s, 2H), 4.35 (m, 2H), 4.00 (m, 1H), 3.96 (m, 2H), 3.21 (m, 2H), 3.01 (m, 2H), 2.80 (t, 2H), 2.48 (s, 2H), 1.69 (t, 2H), 0.90 (s, 6H); MS (EI) for C₂₇H₃₁F₃N₆O₄S: 593 (MH⁺).

5-[4-(6,6-dimethyl-5,6,7,8-tetrahydroquinazolin-4-yl)-2,3,4,5-tetrahydro-1,4-benzoxazepin-7-yl]-3-(hexahydropyrrolo[3,4-c]pyrrol-2(1H)-ylsulfonyl)pyridin-2-amine. Prepared as dihydrochloride salt according to the method of example 5 by using tert-butyl 5-(2-amino-5-bromopyridin-3-ylsulfonyl)hexahydropyrrolo[3,4-c]pyrrole-2(1H)-carboxylate (reagent preparation 25) in step 1 followed by Boc deprotection. ¹H NMR (400 MHz, methanol-d₄): 8.52 (m, 2H), 8.41 (m, 1H), 7.67 (s, 1H), 7.50 (s, 1H), 7.07 (d, 1H), 5.16 (s, 2H), 4.47 (m, 2H), 4.32 (m, 2H), 3.57 (m, 2H), 3.48 (m, 2H), 3.30 (m, 2H), 3.14 (m, 4H), 2.86 (t, 2H), 2.60 (s, 2H), 1.71 (t, 2H), 0.96 (s, 6H); MS (EI) for C₃₀H₃₇N₇O₃S: 576 (MH⁺).

3-[(3-amino-3-methylpyrrolidin-1-yl)sulfonyl]-5-[4-(6,6-dimethyl-5,6,7,8-tetrahydroquinazolin-4-yl)-2,3,4,5-tetrahydro-1,4-benzoxazepin-7-yl]pyridin-2-amine. Prepared as dihydrochloride salt according to the method of example 5 by using tert-butyl 1-(2-amino-5-bromopyridin-3-ylsulfonyl)-3-methylpyrrolidin-3-ylcarbamate (reagent preparation 25) in step 1 followed by Boc deprotection. ¹H NMR (400 MHz, methanol-d₄): 8.52 (m, 2H), 8.32 (m, 1H), 7.66 (m, 1H), 7.48 (m, 1H), 7.06 (m, 1H), 5.16 (s, 2H), 4.47 (m, 2H), 4.32 (m, 2H), 3.72-3.44 (m, 2H), 2.86 (t, 2H), 2.60 (s, 2H), 2.16 (m, 2H), 1.71 (t, 2H), 1.45 (m, 3H), 0.96 (s, 6H); MS (EI) for C₂₉H₃₇N₇O₃S: 564 (MH⁺)

3-[(1S,4S)-2,5-diazabicyclo[2.2.1]hept-2-ylsulfonyl]-5-[4-(6,6-dimethyl-5,6,7,8-tetrahydroquinazolin-4-yl)-2,3,4,5-tetrahydro-1,4-benzoxazepin-7-yl]pyridin-2-amine. Prepared as diacetate salt according to the method of example 5 by using (1S,4S)-tert-butyl 5-(2-amino-5-bromopyridin-3-ylsulfonyl)-2,5-diazabicyclo[2.2.1]heptane-2-carboxylate (reagent preparation 25) in step 1 followed by Boc deprotection. ¹H NMR (400 MHz, methanol-d₄): 8.49 (d, 1H), 8.34 (s, 1H), 8.15 (d, 1H), 7.50 (d, 1H), 7.42 (dd, 1H), 7.06 (d, 1H), 4.71 (s, 2H), 4.63 (s, 1H), 4.35 (m, 2H), 4.02 (s, 1H), 3.95 (m, 2H), 3.50-3.05 (m, 4H), 2.79 (t, 2H), 2.49 (s, 2H), 1.95 (s, 6H), 1.79-1.63 (m, 4H), 0.90 (s, 6H); MS (EI) for C₂₉H₃₅N₇O₃S: 562 (MH⁺).

3-[(3-amino-3-methylpyrrolidin-1-yl)sulfonyl]-5-{4-[(7S)-7-ethyl-5,6,7,8-tetrahydroquinazolin-4-yl]-2,3,4,5-tetrahydro-1,4-benzoxazepin-7-yl}pyridin-2-amine. Prepared as dihydrochloride salt according to the method of example 5 by using {4-[(7S)-7-ethyl-5,6,7,8-tetrahydroquinazolin-4-yl]-2,3,4,5-tetrahydro-1,4-benzoxazepin-7-yl}boronic acid (reagent preparation 23) and tert-butyl 1-(2-amino-5-bromopyridin-3-ylsulfonyl)-3-methylpyrrolidin-3-ylcarbamate (reagent preparation 25) in step 1 followed by Boc deprotection. ¹H NMR (400 MHz, methanol-d₄): 8.50 (m, 3H), 7.70 (s, 1H), 7.50 (d, 1H), 7.07 (d, 1H), 5.23 (d, 1H), 5.13 (d, 1H), 4.57 (m, 1H), 4.46 (m, 1H), 4.35 (m, 1H), 4.21 (m, 1H), 3.70 (m, 3H), 3.55 (m, 1H), 3.45 (m, 1H), 2.98 (m, 2H), 2.73 (m, 1H), 2.41 (m, 1H), 2.19 (m, 2H), 2.05 (m, 1H), 1.82 (m, 1H), 1.48 (m, 4H), 1.24 (m, 1H), 1.02 (t, 3H); MS (EI) for C₂₉H₃₇N₇O₃S: 564 (MH⁺).

3-[(1S,4S)-2,5-diazabicyclo[2.2.1]hept-2-ylsulfonyl]-5-{4-[(7S)-7-ethyl-5,6,7,8-tetrahydroquinazolin-4-yl]-2,3,4,5-tetrahydro-1,4-benzoxazepin-7-yl}pyridin-2-amine. Prepared as dihydrochloride salt according to the method of example 5 by using {4-[(7S)-7-ethyl-5,6,7,8-tetrahydroquinazolin-4-yl]-2,3,4,5-tetrahydro-1,4-benzoxazepin-7-yl}boronic acid (reagent preparation 23) and (1S,4S)-tert-butyl 5-(2-amino-5-bromopyridin-3-ylsulfonyl)-2,5-diazabicyclo[2.2.1]heptane-2-carboxylate (reagent preparation 25) in step 1 followed by Boc deprotection. ¹H NMR (400 MHz, methanol-d₄): 8.65 (s, 1H), 8.52 (s, 2H), 7.74 (s, 1H), 7.53 (d, 1H), 7.08 (d, 1H), 5.24 (d, 1H), 5.14 (d, 1H), 4.56 (m, 2H), 4.46 (m, 1H), 4.36 (m, 1H), 4.21 (m, 1H), 3.69 (m, 3H), 3.53 (m, 1H), 3.38 (m, 1H), 2.99 (m, 2H), 2.74 (m, 1H), 2.42 (m, 1H), 2.05 (m, 3H), 1.83 (m, 1H), 1.47 (m, 2H), 1.24 (m, 1H), 1.01 (t, 3H); MS (EI) for C₂₉H₃₅N₇O₃S: 562 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-[(2S)-pyrrolidin-2-ylmethyl]pyridin-3-sulfonamide. Prepared as dihydrochloride salt according to the method of example 5 by using (R)-tert-butyl 2-((2-amino-5-bromopyridine-3-sulfonamido)methyl)pyrrolidine-1-carboxylate (reagent preparation 25) in step 1 followed by Boc deprotection. ¹H NMR (400 MHz, methanol-d₄): 8.66-8.48 (m, 3H), 7.75 (m, 1H), 7.52 (m, 1H), 7.06 (d, 1H), 5.16 (s, 2H), 4.48 (m, 2H), 4.31 (m, 2H), 3.73 (m, 1H), 2.86 (t, 2H), 2.61 (s, 2H), 2.17 (m, 1H), 2.07 (m, 2H), 1.72 (m, 3H), 0.95 (s, 6H); MS (EI) for C₂₉H₃₇N₇O₃S: 564 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-[(2R)-pyrrolidin-2-ylmethyl]pyridine-3-sulfonamide. Prepared as dihydrochloride salt according to the method of example 5 by using (S)-tert-butyl 2-((2-amino-5-bromopyridine-3-sulfonamido)methyl)pyrrolidine-1-carboxylate (reagent preparation 25) in step 1 followed by Boc deprotection. ¹H NMR (400 MHz, methanol-d₄): 8.78 (s, 1H), 8.54 (s, 1H), 8.49 (s, 1H), 7.84 (s, 1H), 7.56 (m, 1H), 7.07 (d, 1H), 5.18 (s, 2H), 4.49 (m, 2H), 4.31 (m, 2H), 3.75 (m, 1H), 3.38 (m, 2H), 2.86 (m, 2H), 2.62 (s, 2H), 2.20 (m, 1H), 2.08 (m, 2H), 1.74 (m, 3H), 0.95 (s, 6H); MS (EI) for C₂₉H₃₇N₇O₃S: 564 (MH⁺).

3-(2,6-diazaspiro[3.3]hept-2-ylsulfonyl)-5-[4-(6,6-dimethyl-5,6,7,8-tetrahydroquinazolin-4-yl)-2,3,4,5-tetrahydro-1,4-benzoxazepin-7-yl]pyridin-2-amine. Prepared as diacetate salt according to the method of example 5 by using tert-butyl 6-(2-amino-5-bromopyridin-3-ylsulfonyl)-2,6-diazaspiro[3.3]heptane-2-carboxylate (reagent preparation 25) in step 1 followed by Boc deprotection. ¹H NMR (400 MHz, methanol-d₄): 8.52 (d, 1H), 8.33 (s, 1H), 8.09 (d, 1H), 7.50 (d, 1H), 7.43 (dd, 1H), 7.06 (d, 1H), 4.71 (s, 2H), 4.34 (m, 2H), 4.06 (s, 8H), 3.96 (m, 2H), 2.80 (t, 2H), 2.49 (s, 2H), 1.93 (s, 2H), 1.93 (s, 6H), 1.69 (t, 2H), 0.92 (s, 6H); MS (EI) for C₂₉H₃₅N₇O₃S: 562 (MH⁺).

3-[(1R,4R)-2,5-diazabicyclo[2.2.1]hept-2-ylsulfonyl]-5-[4-(6,6-dimethyl-5,6,7,8-tetrahydroquinazolin-4-yl)-2,3,4,5-tetrahydro-1,4-benzoxazepin-7-yl]pyridin-2-amine. Prepared as dihydrochloride salt according to the method of example 5 by using (1R,4R)-tert-butyl 5-(2-amino-5-bromopyridin-3-ylsulfonyl)-2,5-diazabicyclo[2.2.1]heptane-2-carboxylate (reagent preparation 25) in step 1 followed by Boc deprotection. ¹H NMR (400 MHz, methanol-d₄): 8.53 (m, 2H), 8.29 (m, 1H), 7.64 (s, 1H), 7.48 (d, 1H), 7.05 (d, 1H), 5.15 (s, 2H), 4.47 (m, 2H), 4.31 (m, 2H), 3.57 (m, 2H), 3.50 (m, 1H), 2.85 (t, 2H), 2.61 (s, 2H), 1.95 (m, 2H), 1.71 (t, 2H), 0.90 (s, 6H); MS (EI) for MS (EI) for C₂₉H₃₅N₇O₃S: 562 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-piperidin-4-ylpyridine-3-sulfonamide. Prepared as dihydrochloride salt according to the method of example 5 by using tert-butyl 4-(2-amino-5-bromopyridine-3-sulfonamido)piperidine-1-carboxylate (reagent preparation 25) in step 1 followed by Boc deprotection. MS (EI) for C₂₉H₃₇N₇O₃S: 564 (MH⁺).

5-[4-(6,6-dimethyl-5,6,7,8-tetrahydroquinazolin-4-yl)-2,3,4,5-tetrahydro-1,4-benzoxazepin-7-yl]-3-{[(1S,4S)-5-methyl-2,5-diazabicyclo[2.2.2.1]hept-2-yl]sulfonyl}pyridin-2-amine. Prepared as dihydrochloride salt according to the method of example 5 by using 5-bromo-3-((1S,4S)-5-methyl-2,5-diazabicyclo[2.2.1]heptan-2-ylsulfonyl)pyridin-2-amine (reagent preparation 25) in step 1. ¹H NMR (400 MHz, methanol-d₄): 8.48 (d, 1H), 8.34 (s, 1H), 8.14 (d, 1H), 7.49 (d, 1H), 7.42 (dd, 1H), 7.06 (d, 1H), 4.70 (s, 2H), 4.51 (s, 1H), 4.34 (m, 2H), 3.95 (m, 2H), 3.69 (s, 1H), 3.53 (d, 1H), 3.28 (m, 1H), 3.00 (d, 1H), 2.92 (m, 1H), 2.79 (t, 2H), 2.50 (s, 3H), 2.48 (s, 2H), 1.96 (s, 6H), 1.95 (m, 1H), 1.68 (t, 2H), 1.58 (d, 1H), 0.91 (s, 3H), 0.90 (s, 3H); MS (EI) for MS (EI) for C₃₀H₃₇N₇O₃S: 576 (MH⁺).

3-{[(3R)-3-aminopyrrolidin-1-yl]sulfonyl}-5-[4-(6,6,8-trimethyl-5,6-dihydroquinazolin-4-yl)-2,3,4,5-tetrahydro-1,4-benzoxazepin-7-yl]pyridin-2-amine. Synthesized according to the method of example 5 using (R)-tert-butyl 1-(2-amino-5-bromopyridin-3-ylsulfonyl)pyrrolidin-3-ylcarbamate (reagent preparation 25) and [4-(6,6,8-trimethyl-5,6-dihydroquinazolin-4-yl)-2,3,4,5-tetrahydro-1,4-benzoxazepin-7-yl]boronic acid (reagent preparation 23) in step 1 and BOC group deprotection. ¹H NMR (400 MHz, d₆-DMSO): 8.54 (s, 1H), 8.48 (br s, 1H), 8.01 (d, 1H), 8.58 (d, 1H), 7.47 (dd, 1H), 7.02 (d, 1H), 6.76 (br s, 2H), 5.99 (d, 1H), 4.60 (s, 2H), 4.32 (m, 2H), 383 (m, 2H), 3.40-3.32 (m, 4H), 3.30-3.26 (m, 2H), 2.90 (m, 1H), 2.70 (s, 2H), 1.97 (s, 3H), 1.89 (s, 6H), 1.63 (m, 1H), 0.93 (s, 6H); MS (EI) for C₂₉H₃₅N₇O₃S: 562 (MH⁺).

4-(6,6-dimethyl-5,6,7,8-tetrahydroquinazolin-4-yl)-7-(2-methyl-3H-imidazo[4,5-c]pyridin-7-yl)-2,3,4,5-tetrahydro-1,4-benzoxazepine. Synthesized according to the method of example 5 using 7-bromo-3-((2-methoxyethoxy)methyl)-2-methyl-3H-imidazo[4,5-c]pyridine (reagent preparation 19) in step 1. ¹H NMR (400 MHz, d₆-DMSO): 8.72 (s, 1H), 8.48 (br s, 1H) 8.36 (s, 1H), 8.11 (br s, 1H), 7.62 (br m, 1H), 7.22 (br d, 1H), 4.67 (s, 2H), 4.36 (m, 2H), 3.88 (m, 2H), 2.70 (t, 2H), 2.57 (s, 3H), 2.47 (s, 2H), 1.59 (t, 2H), 0.85 (s, 6H); MS (EI) for C₂₆H₂₈N₆O: 441 (MH⁺).

2-amino-N-[(3R)-1-methylpyrrolidin-3-yl]-5-[4-(2,6,6-trimethyl-5,6,7,8-tetrahydroquinazolin-4-yl)-2,3,4,5-tetrahydro-1,4-benzoxazepin-7-yl]pyridine-3-sulfonamide. Synthesized according to the method of example 5 using (S)-2-amino-5-bromo-N-(1-methylpyrrolidin-3-yl)pyridine-3-sulfonamide (reagent preparation 25) and [4-(2,6,6-trimethyl-5,6,7,8-tetrahydroquinazoinazolin-4-yl)-2,3,4,5-tetrahydro-1,4-benzoxazepin-7-yl]boronic acid (reagent preparation 23) in step 1. ¹H NMR (400 MHz, d₆-DMSO): 8.52 (d, 1H), 8.07 (d, 1H), 7.61 (d, 1H), 7.45 (dd, 1H), 7.02 (d, 1H), 6.68 (br s, 2H), 4.57 (s, 2H), 4.25 (br t, 2H), 3.82 (br m, 2H), 3.63 (br m, 1H), 2.65 (t, 2H), 2.43 (m, 4H), 2.33 (s, 3H), 2.22 (br m, 2H), 2.13 (s, 3H), 1.84 (s, 5H), 1.57 (t, 2H), 1.45 (m, 1H), 0.83 (s, 6H); MS (EI) for C₃₀H₃₉N₇O₃S: 578 (MH⁺).

2-amino-N-[(3R)-1-methylpyrrolidin-3-yl]-5-[4-(6,6,8-trimethyl-5,6-dihydroquinazolin-4-yl)-2,3,4,5-tetrahydro-1,4-benzoxazepin-7-yl]pyridine-3-sulfonamide. Synthesized according to the method of example 5 using (S)-2-amino-5-bromo-N-(1-methylpyrrolidin-3-yl)pyridine-3-sulfonamide (reagent preparation 25) and [4-(6,6,8-trimethyl-5,6-dihydroquinazolin-4-yl)-2,3,4,5-tetrahydro-1,4-benzoxazepin-7-yl]boronic acid (reagent preparation 23) in step 1. ¹H NMR (400 MHz, d₆-DMSO): 8.52 (d, 1H), 8.37 (s, 1H), 8.12 (s, H), 8.06 (d, 1H), 7.56 (d, 1H), 7.45 (dd, 1H), 7.02 (d, 1H), 6.69 (br s, 2H), 6.12 (s, 1H), 4.62 (s, 2H), 4.32 (br t, 2H), 3.84 (br m, 2H), 3.63 (br s, 1H), 2.68 (s, 2H), 2.44 (m, 2H), 2.26-2.16 (m, 2H), 2.14 (s, 3H), 1.88 (s, 3H), 1.86 (s, 2H), 1.48-1.42 (m, 1H), 0.91 (s, 6H); MS (EI) for C₃₀H₃₇N₇O₃S: 576 (MH⁺).

3-{[(3S)-3-aminopyrrolidin-1-yl]sulfonyl}-5-{4-[7-(methyloxy)quinazolin-4-yl]-2,3,4,5-tetrahydro-1,4-benzoxazepin-7-yl}pyridin-2-amine. Synthesized according to the method of example 5 using (S)-tert-butyl 1-(2-amino-5-bromopyridin-3-ylsulfonyl)pyrrolidin-3-ylcarbamate (reagent preparation 25) and {4-[7-(methyloxy)quinazolin-4-yl]-2,3,4,5-tetrahydro-1,4-benzoxazepin-7-yl}boronic acid (reagent preparation 23) in step 1 and BOC group deprotection. ¹H NMR (400 MHz, d₆-DMSO): 8.80 (s, 1H), 8.65 (d, 1H), 8.41 (m, 2H), 8.22 (m, 1H), 8.04 (d, 1H), 7.78 (s, 1H), 7.53 (d, 1H), 7.35 (m, 2H), 6.98 (br m, 2H), 5.43 (S, 2H), 4.62 (s, 2H), 4.49 (s, 2H), 3.92 (s, 3H), 3.79 (m, 1H), 3.69 (m, 1H), 3.55-3.45 (m, 3H), 3.32 (m, 2H), 2.20 (m, 1H), 1.91 (M, 1H); MS (EI) for C₂₇H₂₉N₇O₄S: 548 (MH⁺).

N-(2-chloro-5-(4-[7-(methyloxy)quinazolin-4-yl]-2,3,4,5-tetrahydro-1,4-benzoxazepin-7-yl)pyridin-3-yl)methanesulfonamide. Synthesized according to the method of example 5 using N-(5-bromo-2-chloropyridin-3-yl)methanesulfonamide (reagent preparation 25) and {4-[7-(methyloxy)quinazolin-4-yl]-2,3,4,5-tetrahydro-1,4-benzoxazepin-7-yl}boronic acid (reagent preparation 23) in step 1. ¹H NMR (400 MHz, d₆-DMSO): 8.92 (br s, 1H), 8.75 (s, 1H), 8.62 (d, 1H), 8.19 (d, 1H), 8.10 (d, 1H), 7.89 (s, 1H), 7.62 (dd, 1H), 7.33 (dd, 1H), 7.25 (d, 1H), 7.04 (d, 1H), 5.41 (s, 2H), 4.65 (s, 2H), 4.47 (s, 2H), 3.96 (s, 3H), 3.19 (s, 3H); MS (EI) for C₂₄H₂₂ClN₅O₄S: 514 (MH⁺).

N-[6-(4-{2-[(dimethylamino)methyl]-7-(methyloxy)quinazolin-4-yl}-2,3,4,5-tetrahydro-1,4-benzoxazepin-7-yl)[1,3]thiazolo[5,4-b]pyridin-2-yl]acetamide. Synthesized according to the method of example 5 using N-(6-bromothiazolo[5,4-b]pyridin-2-yl)acetamide (J. Heterocyclic Chemistry 2003, 40, 261) and {4-[7-(methyloxy)quinazolin-4-yl]-2,3,4,5-tetrahydro-1,4-benzoxazepin-7-yl}boronic acid (reagent preparation 23) in step 1. ¹H NMR (400 MHz, d₆-DMSO) δ 12.60 (s, 1H), 8.86 (d, 1H), 8.45 (d, 1H), 8.23-8.07 (m, 2H), 7.70 (dd, 1H), 7.27-7.17 (m, 2H), 7.02 (d, 1H), 5.43 (s, 2H), 4.65-4.57 (m, 2H), 4.46 (s, 2H), 4.43-4.37 (m, 2H), 3.94 (s, 3H), 2.73 (s, 6H), 2.25 (s, 3H); MS (EI) for C₂₉H₂₉N₇O₃S: 556 (MH⁺).

(3S)-1-({2-chloro-5-[4-(6,6-dimethyl-5,6,7,8-tetrahydroquinazolin-4-yl)-2,3,4,5-tetrahydro-1,4-benzoxazepin-7-yl]pyridin-3-yl}sulfonyl)pyrrolidin-3-amine. Prepared as in example 5 using (R)-tert-butyl 1-(2-amino-5-bromopyridin-3-ylsulfonyl)pyrrolidin-3-ylcarbamate (reagent preparation 25) in step 1 and BOC group deprotection. ¹H NMR (400 MHz, CD₃OD) δ 8.83 (d, 1H), 8.59 (d, 1H), 8.33 (s, 1H), 7.68 (d, 1H), 7.58 (dd, 1H), 7.12 (d, 1H), 4.75 (s, 2H), 4.44-4.33 (m, 2H), 4.01-3.92 (m, 2H), 3.75-3.42 (m, 4H), 3.23 (dd, 1H), 2.79 (t, 2H), 2.47 (s, 2H), 2.28-2.09 (m, 1H), 1.88-1.77 (m, 1H), 1.69 (t, 2H), 0.90 (s, 6H); MS (ES) for C₂₈H₃₃ClN₆O₃S: 569.2 (MH⁺).

3-{[(3R)-3-aminopyrrolidin-1-yl]sulfonyl}-5-[4-(6,6,7-trimethyl-5,6-dihydroquinazolin-4-yl)-2,3,4,5-tetrahydro-1,4-benzoxazepin-7-yl]pyridin-2-amine. Prepared as in example 5 using (R)-tert-butyl 1-(2-amino-5-bromopyridin-3-ylsulfonyl)pyrrolidin-3-ylcarbamate (reagent preparation 25) and [4-(6,6,7-trimethyl-5,6-dihydroquinazolin-4-yl)-2,3,4,5-tetrahydro-1,4-benzoxazepin-7-yl]boronic acid (reagent preparation 23) in step 1 and BOC group deprotection. ¹H NMR (400 MHz, d₆-DMSO) δ 8.54 (s, 1H), 8.37 (s, 1H), 8.01 (d, 1H), 7.57 (d, 1H), 7.48 (dd, 1H), 7.02 (d, 1H), 6.76 (s, 2H), 6.13 (d, 1H), 4.62 (s, 2H), 4.33 (s, 2H), 3.84 (s, 2H), 3.44-3.20 (m, 3H), 2.89 (d, 1H), 2.69 (s, 2H), 1.88 (d, 5H), 1.53 (s, 1H), 0.92 (s, 6H); MS (ES) for C₂₉H₃₅N₇O₃S: 562.2 (MH⁺).

2-amino-N-(azetidin-3-ylmethyl)-5-[4-(6,6-dimethyl-5,6,7,8-tetrahydroquinazolin-4-yl)-2,3,4,5-tetrahydro-1,4-benzoxazepin-7-yl]pyridine-3-sulfonamide. The diacetate salt was prepared as in example 5 using tert-butyl 3-((2-amino-5-bromopyridine-3-sulfonamido)methyl)azetidine-1-carboxylate (reagent preparation 25) in step 1 and BOC group deprotection. ¹H NMR (400 MHz, d₆-DMSO) δ 8.52 (d, 1H), 8.37 (s, 1H), 8.06 (d, 1H), 7.60 (d, 1H), 7.46 (dd, 1H), 7.03 (d, 1H), 6.68 (bs, 2H), 4.62 (s, 2H), 4.37-4.24 (m, 2H), 3.87-3.77 (m, 2H), 3.48 (t, 2H), 3.26-3.14 (m, 2H), 2.96 (d, 2H), 2.76-2.56 (m, 3H), 2.45 (s, 2H), 1.60 (t, 2H), 0.84 (s, 6H); MS (ES) for C₂₈H₃₅N₇O₃S: 550.2 (MH⁺).

2-amino-N-(2-amino-2-methylpropyl)-5-[4-(6,6,7-trimethyl-5,6-dihydroquinazolin-4-yl)-2,3,4,5-tetrahydro-1,4-benzoxazepin-7-yl]pyridine-3-sulfonamide. The diacetate salt was prepared as in example 5 using 2-amino-N-(2-amino-2-methylpropyl)-5-bromopyridine-3-sulfonamide (reagent preparation 25) and [4-(6,6,7-trimethyl-5,6-dihydroquinazolin-4-yl)-2,3,4,5-tetrahydro-1,4-benzoxazepin-7-yl]boronic acid (reagent preparation 23) in step 1. ¹H NMR (400 MHz, d₆-DMSO) δ 8.50 (d, 1H), 8.37 (s, 1H), 8.05 (d, 1H), 7.55 (s, 1H), 7.45 (d, 1H), 7.02 (d, 1H), 6.73 (s, 2H), 6.12 (s, 1H), 4.62 (s, 2H), 4.38-4.26 (m, 2H), 3.88-3.79 (m, 2H), 2.69 (s, 2H), 2.60 (s, 2H), 2.50 (s, 3H), 1.02-0.83 (m, 12H); MS (ES) for C₂₉H₃₇N₇O₃S: 564.3 (MH⁺).

7-(3,4-dihydro-2H-pyrido[3,2-b][1,4]oxazin-7-yl)-4-(6,6-dimethyl-5,6,7,8-tetrahydroquinazolin-4-yl)-2,3,4,5-tetrahydro-1,4-benzoxazepine. Synthesized according to the method of example 5 using 7-bromo-3,4-dihydro-2H-pyrido[3,2-b][1,4]oxazine in step 1. ¹H NMR (400 MHZ, DMSO-d₆): 8.37 (s, 1H), 7.89 (d, 1H), 7.54 (d, 1H), 7.39 (dd, 1H), 7.22 (d, 1H), 6.97 (d, 1H), 6.83 (s, 1H), 4.57 (s, 2H), 4.29 (br s, 2H), 4.14 (tr, 2H), 3.82 (br s, 2H), 3.42 (br s, 2H), 2.71 (tr, 2H), 2.45 (tr, 2H), 1.60 (tr, 2H), 0.85 (s, 6H); MS (EI) for C₂₆H₂₉N₅O₂: 444 (MH⁺).

5-[4-(6,6-dimethyl-5,6,7,8-tetrahydroquinazolin-4-yl)-2,3,4,5-tetrahydro-1,4-benzoxazepin-7-yl]pyrimidin-2-amine. Synthesized according to the method of example 5 using 5-bromo-2-aminopyrimidine in step 1. ¹H NMR (400 MHZ, DMSO-d₆): 8.54 (s, 2H), 8.37 (s, 1H), 7.59 (d, 1H), 7.44 (dd, 1H), 7.00 (d, 1H), 6.75 (s, 2H), 4.59 (s, 2H), 4.30 (br s, 2H), 3.83 (br s, 2H), 2.71 (tr, 2H), 2.45 (s, 2H), 1.60 (tr, 2H), 0.84 (s, 6H); MS (EI) for C₂₃H₂₆N₆O: 403 (MH⁺).

(2E)-5-[4-(6,6-dimethyl-5,6,7,8-tetrahydroquinazolin-4-yl)-2,3,4,5-tetrahydro-1,4-benzoxazepin-7-yl]-2-iminopyrimidin-1(2H)-ol. Synthesized according to the method of example 5 using 5-bromo-2-iminopyrimidin-1(2H)-ol in step 1. ¹H NMR (400 MHZ, CD₃OD): 8.59 (d, 1H), 8.38 (d, 1H), 8.34 (s, 1H), 7.55 (d, 1H), 7.44 (dd, 1H), 7.08 (d, 1H), 4.72 (s, 2H), 4.35 (tr, 2H), 3.95 (tr, 2H), 2.79 (tr, 2H), 2.46 (s, 2H), 1.68 (tr, 2H), 0.90 (s, 6H); MS (EI) for C₂₃H₂₆N₆O₂: 419 (MH⁺).

5-[4-(6,6-dimethyl-5,6,7,8-tetrahydroquinazolin-4-yl)-2,3,4,5-tetrahydro-1,4-benzoxazepin-7-yl]-3-(ethylsulfonyl)pyridin-2-amine. Synthesized according to the method of example 5 using 5-bromo-3-(ethylsulfonyl)pyridin-2-amine (reagent preparation 34) in step 1. ¹H NMR (400 MHZ, CD₃CN): 8.56 (d, 1H), 8.37 (s, 1H), 8.10 (d, 1H), 7.50 (d, 1H), 7.45 (dd, 1H), 7.06 (d, 1H), 6.13 (s, 2H), 4.64 (s, 2H), 4.34 (tr, 2H), 3.90 (tr, 2H), 3.24 (q, 2H), 2.78 (tr, 2H), 2.46 (s, 2H), 1.66 (tr, 2H), 1.23 (tr, 3H), 0.90 (s, 6H). MS (EI) for C₂₆H₃N₅SO₃: 494 (MH⁺).

3-({2-amino-5-[4-(6,6-dimethyl-5,6,7,8-tetrahydroquinazolin-4-yl)-2,3,4,5-tetrahydro-1,4-benzoxazepin-7-yl]pyridin-3-yl}sulfonyl)propane-1,2-diol. Synthesized according to the method of example 5 using 3-(2-amino-5-bromopyridin-3-ylsulfonyl)propane-1,2-diol in step 1. ¹H NMR (400 MHZ, CD₃OD): 8.49 (d, 1H), 8.33 (s, 1H), 8.18 (d, 1H), 7.50 (d, 1H), 7.42 (dd, 1H), 7.04 (d, 1H), 4.69 (s, 2H), 4.34 (tr, 2H), 4.15-4.06 (m, 1H), 3.95 (tr, 2H), 3.54-3.37 (m, 4H), 2.79 (tr, 2H), 2.48 (s, 2H), 1.68 (tr, 2H), 0.90 (s, 6H). MS (EI) for C₂₇H₃₃N₅SO₅: 540 (MH⁺).

3-({2-amino-5-[4-(6,6-dimethyl-5,6,7,8-tetrahydroquinazolin-4-yl)-2,3,4,5-tetrahydro-1,4-benzoxazepin-7-yl]pyridin-3-yl}sulfonyl)propan-1-ol. Synthesized according to the method of example 5 using 3-(2-amino-5-bromopyridin-3-ylsulfonyl)propan-1-ol (reagent preparation 34) in step 1. ¹H NMR (400 MHZ, CD₃OD): 8.52 (d, 1H), 8.34 (s, 1H), 8.14 (d, 1H), 7.50 (d, 1H), 7.42 (dd, 1H), 7.06 (d, 1H), 4.69 (d, 2H), 4.34 (tr, 2H), 3.95 (tr, 2H), 3.61 (tr, 2H), 3.37-3.33 (m, 2H), 2.80 (tr, 2H), 2.48 (s, 2H), 1.94-1.86 (m, 2H), 1.69 (tr, 2H), 0.90 (s, 6H). MS (EI) for C₂₇H₃₃N₅SO₄: 524 (MH⁺).

(2S)-3-({2-amino-5-[4-(6,6-dimethyl-5,6,7,8-tetrahydroquinazolin-4-yl)-2,3,4,5-tetrahydro-1,4-benzoxazepin-7-yl]pyridin-3-yl}sulfonyl)-2-methylpropan-1-ol. Synthesized according to the method of example 5 using (S)-3-(2-amino-5-bromopyridin-3-ylsulfonyl)-2-methylpropan-1-ol (reagent preparation 34) in step 1. ¹H NMR (400 MHZ, CD₃OD): 8.51 (d, 1H), 8.33 (s, 1H), 8.15 (d, 1H), 7.50 (d, 1H), 7.42 (dd, 1H), 7.05 (d, 1H), 4.69 (s, 2H), 4.34 (tr, 2H), 3.95 (tr, 2H), 3.52-3.37 (m, 3H), 3.06 (dd, 1H), 2.79 (tr, 2H), 2.48 (s, 2H), 2.23-2.16 (m, 1H), 1.68 (tr, 2H), 1.08 (d, 3H), 0.90 (s, 6H). MS (EI) for C₂₈H₃₅N₅SO₄: 538 (MH⁺).

(2R)-3-({2-amino-5-[4-(6,6-dimethyl-5,6,7,8-tetrahydroquinazolin-4-yl)-2,3,4,5-tetrahydro-1,4-benzoxazepin-7-yl]pyridin-3-yl}sulfonyl)-2-methylpropan-1-ol. Synthesized according to the method of example 5 using (R)-3-(2-amino-5-bromopyridin-3-ylsulfonyl)-2-methylpropan-1-ol (reagent preparation 34) in step 1. ¹H NMR (400 MHZ, CD₃OD): 8.51 (d, 1H), 8.33 (s, 1H), 8.15 (d, 1H), 7.50 (d, 1H), 7.42 (dd, 1H), 7.05 (d, 1H), 4.69 (s, 2H), 4.34 (tr, 2H), 3.95 (tr, 2H), 3.52-3.37 (m, 3H), 3.06 (dd, 1H), 2.79 (tr, 2H), 2.48 (s, 2H), 2.23-2.16 (m, 1H), 1.68 (tr, 2H), 1.08 (d, 3H), 0.90 (s, 6H). MS (EI) for C₂₈H₃₅N₅SO₄: 538 (MH⁺).

7-[4-(6,6-dimethyl-5,6,7,8-tetrahydroquinazolin-4-yl)-2,3,4,5-tetrahydro-1,4-benzoxazepin-7-yl]-2H-pyrido[2,3-e][1,2,4]thiadiazin-3(4H)-one 1,1-dioxide. Synthesized according to the method of example 5 using 7-bromo-2H-pyrido-[2,3-e][1,2,4]-thiadiazin-3(4H)-one 1,1-dioxide (reagent preparation 37) in step 1. ¹H NMR (400 MHZ, CD₃OD): 8.66 (d, 1H), 8.39 (s, 1H), 8.24 (d, 1H), 7.61 (d, 1H), 7.50 (dd, 1H), 7.07 (d, 1H), 4.81 (s, 2H), 4.38 (tr, 2H), 4.04 (tr, 2H), 2.80 (tr, 2H), 2.51 (tr, 2H), 1.68 (tr, 2H), 0.91 (s, 6H). MS (EI) for C₂₅H₂₆N₆SO₄: 507 (MH⁺).

2-amino-5-[4-(6,6-dimethyl-5,6,7,8-tetrahydroquinazolin-4-yl)-2,3,4,5-tetrahydro-1,4-benzoxazepin-7-yl]pyridine-3-sulfonic acid. Synthesized according to the method of example 5 using 2-amino-5-bromopyridine-3-sulfonic acid (reagent preparation 38) in step 1. ¹H NMR (400 MHZ, CD₃OD): 8.37 (s, 1H), 8.24 (d, 1H), 8.20 (d, 1H), 7.49 (d, 1H), 7.40 (dd, 1H), 7.03 (d, 1H), 4.74 (s, 2H), 4.35 (tr, 2H), 3.99 (tr, 2H), 2.80 (tr, 2H), 2.49 (s, 2H), 1.68 (tr, 2H), 0.90 (s, 6H). MS (EI) for C₂₄H₂₇N₅SO₄: 482 (MH⁺).

N-{5-[4-(6,6-dimethyl-5,6,7,8-tetrahydroquinazolin-4-yl)-2,3,4,5-tetrahydro-1,4-benzoxazepin-7-yl]-2-(phenylamino)pyridin-3-yl}methanesulfonamide. Synthesized according to the method of example 5 using N-(5-bromo-2-(phenylamino)pyridin-3-yl)methanesulfonamide (reagent preparation 39) in step 1. ¹H NMR (400 MHZ, DMSO-d): 9.27 (br s, 1H), 8.35 (s, 2H), 8.30 (s, 1H), 7.78 (s, 1H), 7.67 (d, 2H), 7.57 (s, 1H), 7.44 (d, 1H), 7.27 (s, 2H), 7.01 (d, 1H), 6.94 (s, 1H), 4.62 (br s, 2H), 4.30 (br s, 2H), 3.82 (br s, 2H), 3.05 (s, 3H), 2.69 (br s, 2H), 2.42 (s, 2H), 1.58 (br s, 2H), 0.83 (s, 6H). MS (EI) for C₃₁H₃₄N₆SO₃: 572 (MH⁺).

N-{2-(dimethylamino)-5-[4-(6,6-dimethyl-5,6,7,8-tetrahydroquinazolin-4-yl)-2,3,4,5-tetrahydro-1,4-benzoxazepin-7-yl]pyridin-3-yl}methanesulfonamide. Synthesized according to the method of example 5 using N-(5-bromo-2-(dimethylamino)pyridin-3-yl)methanesulfonamide (reagent preparation 39) in step 1. ¹H NMR (400 MHZ, DMSO-d₆): 9.09 (br s, 1H), 8.36 (s, 1H), 8.35 (d, 1H), 7.71 (d, 1H), 7.56 (d, 1H), 7.44 (dd, 1H), 7.03 (d, 1H), 4.64 (s, 2H), 4.32 (br, 2H), 3.84 (br, 1H), 3.11 (s, 3H), 2.94 (s, 6H), 2.71 (tr, 2H), 2.44 (s, 2H), 1.60 (tr, 2H), 0.85 (s, 6H). MS (EI) for C₂₇H₃₄N₆SO₃: 523 (MH⁺).

3-[(4-aminopiperidin-1-yl)sulfonyl]-5-[4-(6,6-dimethyl-5,6,7,8-tetrahydroquinazolin-4-yl)-2,3,4,5-tetrahydro-1,4-benzoxazepin-7-yl]pyridin-2-amine. Prepared as a dihydrochloride salt according to the method of example 5 by using tert-butyl 1-(2-amino-5-bromopyridin-3-ylsulfonyl)piperidin-4-ylcarbamate (reagent preparation 25) in step 1 followed by Boc deprotection. ¹H NMR (400 MHz, DMSO-d6) δ 8.72 (s, 1H), 8.66 (d, 1H), 8.39 (br s, 3H), 8.10 (d, 1H), 7.73 (s, 1H), 7.54 (d, 1H), 7.00 (d, 1H), 5.11 (s, 2H), 4.53-4.45 (m, 2H), 4.37-4.22 (m, 4H, buried), 3.78 (d, 2H), 3.14 (br s, 1H), 2.86-2.78 (m, 2H), 2.73 (t, 2H), 2.53 (s, 2H), 2.00 (d, 2H), 1.68-1.52 (m, 4H), 0.85 (s, 6H); MS (EI) for C₂₉H₃₇N₇O₃S: 564 (MH⁺).

2-amino-5-[4-(6,6-dimethyl-5,6,7,8-tetrahydroquinazolin-4-yl)-2,3,4,5-tetrahydro-1,4-benzoxazepin-7-yl]-N-(2-hydroxy-2-methylpropyl)pyridine-3-sulfonamide. Prepared according to the method of example 5 by using 2-amino-5-bromo-N-(2-hydroxy-2-methylpropyl)pyridine-3-sulfonamide (reagent preparation 25) in step 1. ¹H NMR (400 MHz, DMSO-d6) δ 8.50 (d, 1H), 8.37 (s, 1H), 8.05 (d, 1H), 7.71 (t, 1H), 7.58 (d, 1H), 7.45 (dd, 1H), 7.02 (d, 1H), 6.74 (br s, 2H), 4.62 (s, 2H), 4.31 (s, 2H), 3.83 (s, 2H), 2.71 (t, 2H), 2.64 (d, 2H), 2.44 (s, 2H), 1.59 (t, 2H), 1.04 (s, 6H), 0.84 (s, 6H); MS (EI) for C₂₈H₃₆N₆O₄S: 553 (MH⁺).

6-[4-(6,6-dimethyl-5,6,7,8-tetrahydroquinazolin-4-yl)-2,3,4,5-tetrahydro-1,4-benzoxazepin-7-yl]-N-ethyl-1H-imidazo[4,5-b]pyridin-2-amine. Prepared as in example 5 using 6-bromo-N-ethyl-3-(methoxymethyl)-3H-imidazo[4,5-b]pyridin-2-amine (reagent preparation 36) in step 1 and MOM group deprotection. ¹H NMR (400 MHz, CD₃OD) δ 8.53 (s, 1H), 8.29 (d, 1H), 7.99 (d, 1H), 7.72 (d, 1H), 7.53 (dd, 1H), 7.07 (d, 1H), 5.17 (s, 2H), 4.48 (m, 2H), 4.32 (m, 2H), 3.56 (q, 2H), 2.86 (t, 2H), 2.61 (s, 2H), 1.70 (t, 2H), 1.36 (t, 3H); MS (ES) for C₂₇H₃₁N₇O: 470 (MH⁺).

1-{4-[7-(1,3-benzothiazol-5-yl)-2,3-dihydro-1,4-benzoxazepin-4(5H)-yl]-6,6-dimethyl-7-(methyloxy)-5,6,7,8-tetrahydroquinazolin-2-yl}-N,N-dimethylmethanamine. The trifluoroacetate salt was synthesized according to the method of example 5 using 5-bromobenzo[d]thiazole and (4-{2-[(dimethylamino)methyl]-7-methoxy-6,6-dimethyl-5,6,7,8-tetrahydroquinazolin-4-yl}-2,3,4,5-tetrahydro-1,4-benzoxazepin-7-yl)boronic acid (reagent preparation 23) in step 1. ¹H NMR (400 MHz, d₆-DMSO) δ 9.45 (s, 1H), 8.36 (d, 1H), 8.26 (d, 1H), 7.81 (m, 2H), 7.64 (dd, 1H), 7.05 (d, 1H), 4.84 (s, 2H), 4.41 (m, 2H), 4.30 (s, 2H), 3.97 (m, 2H), 3.35 (s, 3H), 3.28 (t, 1H), 3.00 (dd, 1H), 2.80 (s, 6H), 2.72 (dd, 1H), 2.62 (d, 1H), 2.44 (d, 1H), 0.85 (d, 6H).; MS (ES) for C₃₀H₃₅N₅O₂S: 530 (MH⁺).

1-{6,6-dimethyl-4-[7-(2-methyl-1H-imidazo[4,5-b]pyridin-6-yl)-2,3-dihydro-1,4-benzoxazepin-4(5H)-yl]-7-(methyloxy)-5,6,7,8-tetrahydroquinazolin-2-yl}-N,N-dimethylmethanamine. The bistrifluoroacetate salt was synthesized according to the method of example 5 using isobutyl 6-bromo-2-methyl-1H-imidazo[4,5-b]pyridine-1-carboxylate (reagent preparation 19) and (4-{2-[(dimethylamino)methyl]-7-methoxy-6,6-dimethyl-5,6,7,8-tetrahydroquinazolin-4-yl}-2,3,4,5-tetrahydro-1,4-benzoxazepin-7-yl)boronic acid (reagent preparation 23) in step 1. ¹H NMR (400 MHz, d₆-DMSO) δ 9.55 (s, 1H), 8.75 (d, 1H), 8.31 (s, 1H), 7.80 (d, 1H), 7.63 (dd, 1H), 7.09 (d, 1H), 4.82 (s, 2H), 4.42 (m, 2H), 4.30 (s, 2H), 3.97 (m, 2H), 3.35 (s, 3H), 3.27 (t, 1H), 3.00 (dd, 1H), 2.81 (s, 6H), 2.72 (m, 1H), 2.71 (s, 3H), 2.61 (d, 1H), 2.43 (d, 1H), 0.83 (d, 6H); MS (ES) for C₃₀H₃₇N₇O₂: 528 (MH⁺).

6-(4-{2-[(dimethylamino)methyl]-7-(methyloxy)quinazolin-4-yl}-2,3,4,5-tetrahydro-1,4-benzoxazepin-7-yl)[1,3]thiazolo[5,4-b]pyridin-2-amine. The dihydrochloride salt was synthesized according to the method of example 5 using N-(6-bromothiazolo[5,4-b]pyridin-2-yl)acetamide (J. Heterocyclic Chem 2003, 40, 261) and (4-{2-[(dimethylamino)methyl]-7-methoxyquinazolin-4-yl}-2,3,4,5-tetrahydro-1,4-benzoxazepin-7-yl)boronic acid (reagent preparation 23) in step 1 and acetyl group hydrolysis. ¹H NMR (400 MHz, d₆-DMSO) δ 8.48 (d, 1H), 8.16-7.96 (m, 4H), 7.94 (d, 1H), 7.60 (dd, 1H), 7.26-7.17 (m, 2H), 7.00 (d, 1H), 5.37 (s, 2H), 4.63-4.53 (m, 2H), 4.43 (s, 2H), 4.41-4.31 (m, 2H), 3.93 (s, 3H), 2.76 (s, 6H); MS (ES) for C₂₇H₂₇N₇O₂S: 514 (MH⁺).

### Example 6

### 1-{6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}-N,N-dimethylmethanamine

STEP 1: A mixture of isobutyl 6-bromo-2-methyl-1H-imidazo[4,5-b]pyridine-1-carboxylate (2.2 g, 7.1 mmol) (reagent preparation 19), (4-{[(1,1-dimethylethyl)-oxy]carbonyl}-2,3,4,5-tetrahydro-1,4-benzoxazepin-7-yl)boronic acid (2.7 g, 9.2 mmol, example 1, step 2), potassium acetate (2.8 g, 28.3 mmol), and dichloro[1,1-bis(diphenylphosphino]ferrocenepalladium (II) dichloromethane adduct (0.78 g, 1.1 mmol) in dioxane (50 ml) was stirred at 95° C. under nitrogen for 29 h. The mixture was cooled to room temperature, filtered through celite, and the filter cake was washed with ethyl acetate (100 ml). The filtrate was concentrated and purified by column chromatography on silica (0-100% ethyl acetate in hexanes) to give 1,1-dimethylethyl 7-(2-methyl-1-{[(2-methylpropyl)oxy]carbonyl}-1H-imidazo[4,5-b]pyridine-6-yl)-2,3-dihydro-1,4-benzoxazepine-4(5H)carboxylate (1.1 g, 33% yield) as a yellow solid. ¹H NMR (400 MHz, CDCl₃): 8.74 (d, 1H), 8.39 (s, 1H), 7.47 (d, 1H), 7.43 (s, 1H), 7.14 (d, 1H), 4.50 (s, 2H), 4.34 (d, 2H), 4.12 (m, 2H), 3.88 (m, 2H), 2.96 (s, 3H), 2.22 (m, 1H), 1.42 (s, 9H), 1.21 (d, 6H); MS (EI) for C₂₆H₃₂N₄O₅: 481 (MH⁺).

STEP 2: A mixture of 1,1-dimethylethyl 7-(2-methyl-1-{[(2-methylpropyl)-oxy]carbonyl}-1H-imidazo[4,5-b]pyridine-6-yl)-2,3-dihydro-1,4-benzoxazepine-4(5H)carboxylate (1.1 g, 2.3 mmol) in methanol (6 ml) and 4 N hydrochloric acid in dioxane (12 ml) was stirred at room temperature for 1 h and then concentrated. The resulting solid was triturated with ethyl acetate to afford the hydrochloride salt of 2-methylpropyl 2-methyl-6-(2,3,4,5-tetrahydro-1,4-benzoxazepin-7-yl)-1H-imidazo[4,5-b]pyridine-1-carboxylate (0.92 g, 91% yield) as a yellow solid. ¹H NMR (400 MHz, DMSO-d₆): 9.78 (s, 1H), 8.79 (d, 1H), 8.42 (d, 1H), 7.88 (d, 1H), 7.70 (dd, 1H), 7.24 (d, 1H), 4.42 (brs, 2H), 4.32 (d, 2H), 4.27 (brs, 2H), 3.50 (brs, 2H), 2.82 (s, 3H), 2.19 (m, 1H), 1.06 (d, 6H); MS (EI) for C₂₁H₂₄N₄O₃: 381 (MH⁺).

STEP 3: A solution of 2-methylpropyl 2-methyl-6-(2,3,4,5-tetrahydro-1,4-benzoxazepin-7-yl)-1H-imidazo[4,5-b]pyridine-1-carboxylate hydrochloride (73 mg, 0.16 mmol), 1-(4-chloro-6,6-dimethyl-5,6,7,8-tetrahydroquinazolin-2-yl)-N,N-dimethylmethanamine (41 mg, 0.16 mmol, reagent preparation 17), and diisopropylethyl amine (0.13 ml, 0.8 mmol) in N-methylpyrrolidinone (1.5 mL) was stirred at 135° C. for 3 h. Purification by preparatory HPLC (0.1% aqueous ammonium acetate-acetonitrile) provided the triacetate salt of the title Compound (10 mg, 9% yield). ¹H NMR (400 MHz, methanol-d₄) δ 8.53 (d, 1H), 8.07 (d, 1H), 7.64 (d, 1H), 7.51 (dd, 1H), 7.08 (d, 1H), 4.79 (s, 2H), 4.38 (s, 2H), 4.01 (s, 2H), 3.84 (s, 2H), 2.81 (t, 2H), 2.64 (s, 3H), 2.55 (s, 6H), 1.92 (s, 9H), 1.68 (t, 2H), 0.91 (s, 6H); MS (EI) for C₂₉H₃₅N₇₀: 498 (MH⁺)

Using analogous synthetic techniques and substituting with alternative starting reagents the following compounds of the invention were prepared. Alternative starting materials were obtained commercially unless otherwise indicated.

N-({6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl)-N-ethylethanamine. Prepared as triacetate salt according to the method of example 6 by using N-((4-chloro-6,6-dimethyl-5,6,7,8-tetrahydroquinazolin-2-yl)methyl)-N-ethylethanamine (reagent preparation 17) in step 3. ¹H NMR (400 MHz, methanol-d₄) δ 8.52 (s, 1H), 8.06 (s, 1H), 7.63 (s, 1H), 7.52 (d, 1H), 7.08 (d, 1H), 4.80 (s, 2H), 4.39 (m, 2H), 4.05 (s, 2H), 4.02 (m, 2H), 3.05 (q, 4H), 2.82 (t, 2H), 2.64 (s, 3H), 2.52 (s, 2H), 1.92 (s, 9H), 1.69 (t, 2H), 1.17 (t, 6H), 0.91 (s, 6H); MS (EI) for C₃₁H₃₉N₇O: 526 (MH⁺).

4-[6,6-dimethyl-2-(morpholin-4-ylmethyl)-5,6,7,8-tetrahydroquinazolin-4-yl]-7-(2-methyl-1H-imidazo[4,5-b]pyridin-6-yl)-2,3,4,5-tetrahydro-1,4-benzoxazepine. Prepared as acetate salt according to the method of example 6 by using 4-((4-chloro-6,6-dimethyl-5,6,7,8-tetrahydroquinazolin-2-yl)methyl)morpholine (reagent preparation 17) in step 3. ¹H NMR (400 MHz, methanol-d₄) δ ¹H NMR (400 MHz, methanol-d₄) δ 8.53 (s, 1H), 8.06 (s, 1H), 7.64 (s, 1H), 7.49 (d, 1H), 7.06 (d, 1H), 4.78 (s, 2H), 4.36 (s, 2H), 4.36 (s, 2H), 4.00 (s, 2H), 3.58 (s, 4H), 3.49 (s, 2H), 2.79 (t, 2H), 2.64 (s, 3H), 2.50 (s, 3H), 2.46 (m, 4H), 1.95 (s, 3H), 1.68 (t, 2H), 0.92 (s, 6H); MS (EI) for C₃₁H₃₇N₇O₂: 540 (MH⁺).

N-({6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl)-N-ethylpropan-2-amine. Prepared according to the method of example 6 by using N-((4-chloro-6,6-dimethyl-5,6,7,8-tetrahydroquinazolin-2-yl)-ethylpropan-2-amine (reagent preparation 17) in step 3. ¹H NMR (400 MHz, methanol-d₄) δ ¹H NMR (400 MHz, methanol-d₄) δ 8.52 (s, 1H), 8.06 (s, 1H), 7.63 (s, 1H), 7.51 (d, 1H), 7.08 (d, 1H), 4.79 (s, 2H), 4.37 (s, 2H), 4.01 (m, 2H), 2.81 (t, 2H), 2.64 (s, 3H), 2.51 (s, 2H), 1.68 (t, 2H), 1.07 (m, 9H), 0.91 (s, 6H); MS (EI) for C₃₂H₄₁N₇O: 540 (MH⁺).

N-({6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl)-2-methylpropan-1-amine. Prepared according to the method of example 6 by using N-((4-chloro-6,6-dimethyl-5,6,7,8-tetrahydroquinazolin-2-yl)methyl)-2-methylpropan-1-amine (reagent preparation 17) in step 3. ¹H NMR (400 MHz, methanol-d₄) δ ¹H NMR (400 MHz, methanol-d₄) δ 8.53 (s, 1H), 8.06 (s, 1H), 7.63 (d, 1H), 7.51 (dd, 1H), 7.07 (d, 1H), 4.81 (s, 2H), 4.37 (s, 2H), 4.02 (m, 2H), 3.78 (s, 2H), 2.79 (t, 2H), 2.64 (s, 3H), 2.51 (s, 2H), 2.43 (s, 2H), 1.68 (m, 3H), 0.93 (s, 6H), 0.82 (d, 6H); MS (EI) for C₃₁H₃₉N₇O: 526 (MH⁺).

N-({6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl)cyclopropanamine. Prepared according to the method of example 6 by using N-((4-chloro-6,6-dimethyl-5,6,7,8-tetrahydroquinazolin-2-yl)methyl)cyclopropanamine (reagent preparation 17) in step 3. ¹H NMR (400 MHz, methanol-d₄) δ ¹H NMR (400 MHz, methanol-d₄) δ 8.57 (s, 1H), 8.10 (s, 1H), 7.70 (d, 1H), 7.51 (dd, 1H), 7.04 (d, 1H), 4.83 (s, 2H), 4.35 (s, 2H), 4.01 (m, 2H), 3.70 (s, 2H), 2.77 (t, 2H), 2.64 (s, 3H), 2.50 (s, 2H), 2.00 (m, 1H), 1.67 (t, 2H), 0.94 (s, 6H), 0.24 (m, 2H), 0.13 (m, 2H); MS (EI) for C₃₀H₃₅N₇O: 510 (MH⁺).

N-({6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl)-2,2-difluoroethanamine. Prepared according to the method of example 6 by using benzyl (4-chloro-6,6-dimethyl-5,6,7,8-tetrahydroquinazolin-2-yl)methyl(2,2-difluoroethyl)carbamate (reagent preparation 17) in step 3 followed by Cbz deprotection. ¹H NMR (400 MHz, methanol-d₄) δ ¹H NMR (400 MHz, methanol-d₄) δ 8.53 (s, 1H), 8.06 (s, 1H), 7.64 (d, 1H), 7.50 (dd, 1H), 7.07 (d, 1H), 5.75 (m, 1H), 4.79 (s, 2H), 4.37 (s, 2H), 4.01 (m, 2H), 3.72 (s, 2H), 2.78 (m, 4H), 2.64 (s, 3H), 2.50 (s, 2H), 1.68 (t, 2H), 0.92 (s, 6H); MS (EI) for C₂₉H₃₃F₂N₇₀: 534 (MH⁺).

N-({6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl)cyclobutanamine. Prepared as acetate salt according to the method of example 6 by using benzyl (4-chloro-6,6-dimethyl-5,6,7,8-tetrahydroquinazolin-2-yl)methyl(cyclobutyl)carbamate (reagent preparation 17) in step 3 followed by Cbz deprotection. ¹H NMR (400 MHz, methanol-d₄) δ ¹H NMR (400 MHz, methanol-d₄) δ 8.54 (s, 1H), 8.07 (s, 1H), 7.65 (s, 1H), 7.52 (d, 1H), 7.08 (d, 1H), 4.82 (s, 2H), 4.38 (m, 2H), 4.03 (m, 2H), 3.82 (m, 2H), 3.51 (m, 1H), 2.80 (t, 2H), 2.64 (s, 3H), 2.51 (s, 2H), 2.07 (m, 2H), 1.92 (s, 3H), 1.83 (m, 2H), 1.68 (m, 4H), 0.92 (s, 6H); MS (EI) for C₃₁H₃₇N₇O: 524 (MH⁺).

{6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl acetate. Prepared according to the method of example 6 by using (4-chloro-6,6-dimethyl-5,6,7,8-tetrahydroquinazolin-2-yl)methyl acetate (reagent preparation 17) in step 3. ¹H NMR (400 MHz, methanol-d₄) δ ¹H NMR (400 MHz, methanol-d₄) δ 8.53 (s, 1H), 8.07 (s, 1H), 7.61 (d, 1H), 7.50 (dd, 1H), 7.09 (d, 1H), 4.99 (s, 2H), 4.74 (s, 2H), 4.34 (m, 2H), 3.99 (m, 2H), 2.78 (t, 2H), 2.64 (s, 3H), 2.50 (s, 2H), 2.08 (s, 3H), 1.67 (t, 2H), 0.92 (s, 6H); MS (EI) for C₂₉H₃₂N₆O₃: 513 (MH⁺).

6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methanol. Prepared from {6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl acetate by ester saponification using standard techniques. ¹H NMR (400 MHz, methanol-d₄) δ ¹H NMR (400 MHz, methanol-d₄) δ 8.54 (s, 1H), 8.08 (s, 1H), 7.65 (d, 1H), 7.50 (dd, 1H), 7.08 (d, 1H), 4.78 (s, 2H), 4.48 (s, 2H), 4.35 (m, 2H), 4.03 (m, 2H), 2.79 (t, 2H), 2.64 (s, 3H), 2.50 (s, 2H), 1.68 (t, 2H), 0.92 (s, 6H); MS (EI) for C₂₇H₃₀N₆O₂: 471 (MH⁺).

N-({6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl)alanine. Prepared as acetate salt according to the method of example 6 by using ethyl 2-((4-chloro-6,6-dimethyl-5,6,7,8-tetrahydroquinazolin-2-yl)methylamino)propanoate (reagent preparation 17) in step 3 followed by saponification using standard techniques. ¹H NMR (400 MHz, methanol-d₄) δ ¹H NMR (400 MHz, methanol-d₄) δ 8.51 (s, 1H), 8.07 (d, 1H), 7.64 (s, 1H), 7.52 (dd, 1H), 7.09 (d, 1H), 4.82 (s, 2H), 4.40 (m, 2H), 4.15 (s, 2H), 4.05 (m, 2H), 3.66 (q, 1H), 2.81 (t, 2H), 2.64 (s, 3H), 2.52 (s, 2H), 1.94 (s, 3H), 1.68 (t, 2H), 1.45 (d, 3H), 0.91 (d, 6H); MS (EI) for C₃₀H₃₅N₇O₃: 542 (MH⁺).

4-[5-(cyclopropylmethyl)-6-methylpyrimidin-4-yl]-7-(2-methyl-1H-imidazo[4,5-b]pyridin-6-yl)-2,3,4,5-tetrahydro-1,4-benzoxazepine. Prepared according to the method of example 6 by using 4-chloro-5-(cyclopropylmethyl)-6-methylpyrimidine (reagent preparation 5) in step 3. ¹H NMR (400 MHz, methanol-d₄) δ 8.51 (s, 1H), 8.35 (s, 1H), 8.05 (s, 1H), 7.61 (d, 1H), 7.51 (dd, 1H), 7.09 (d, 1H), 4.69 (s, 2H), 4.41-4.33 (m, 2H), 3.95-3.86 (m, 2H), 2.70 (d, 2H), 2.63 (s, 3H), 2.48 (s, 3H), 0.96-0.81 (m, 1H), 0.46-0.31 (m, 4H); MS (EI) for C₂₅H₂₆N₆O: 427 (MH⁺).

4-(5-ethyl-6-methylpyrimidin-4-yl)-7-(2-methyl-1H-imidazo[4,5-b]pyridin-6-yl)-2,3,4,5-tetrahydro-1,4-benzoxazepine. Prepared according to the method of example 6 by using 4-chloro-5-ethyl-6-methylpyrimidine in step 3. ¹H NMR (400 MHz, methanol-d₄) δ 8.51 (s, 1H), 8.33 (s, 1H), 8.04 (s, 1H), 7.59 (d, 1H), 7.51 (dd, 1H), 7.09 (d, 1H), 4.71 (s, 2H), 4.37 (m, 2H), 3.90 (m, 2H), 2.74 (q, 2H), 2.64 (s, 3H), 2.44 (s, 3H), 1.20 (t, 3H); MS (EI) for C₂₃H₂₄N₆O: 401 (MH⁺).

4-(5-ethyl-2,6-dimethylpyrimidin-4-yl)-7-(2-methyl-1H-imidazo[4,5-b]pyridin-6-yl)-2,3,4,5-tetrahydro-1,4-benzoxazepine. Prepared according to the method of example 6 by using 4-chloro-5-ethyl-2,6-dimethylpyrimidine (reagent preparation 8) in step 3. ¹H NMR (400 MHz, methanol-d₄) δ 8.86 (d, 1H), 8.44 (d, 1H), 7.83 (d, 1H), 7.61 (dd, 1H), 7.09 (d, 1H), 5.16 (s, 2H), 4.50 (m, 2H), 4.27 (m, 2H), 2.92 (s, 3H), 2.78 (q, 2H), 2.51 (s, 3H), 2.49 (s, 3H), 1.22 (t, 3H); MS (EI) for C₂₄H₂₆N₆O: 415 (MH⁺).

4-[5-(cyclopropylmethyl)-2,6-dimethylpyrimidin-4-yl]-7-(2-methyl-1H-imidazo[4,5-b]pyridin-6-yl)-2,3,4,5-tetrahydro-1,4-benzoxazepine. Prepared according to the method of example 6 by using 4-chloro-5-(cyclopropylmethyl)-2,6-dimethylpyrimidine (reagent preparation 8) in step 3. ¹H NMR (400 MHz, methanol-d₄) δ 8.52 (s, 1H), 8.05 (s, 1H), 7.63 (d, 1H), 7.51 (dd, 1H), 7.09 (d, 1H), 4.66 (s, 2H), 4.33 (m, 2H), 3.91 (m, 2H), 2.67 (d, 2H), 2.64 (s, 3H), 2.44 (s, 3H), 2.42 (s, 3H), 0.87 (m, 1H), 0.38 (m, 4H); MS (EI) for C₂₆H₂₈N₆O: 441 (MH⁺).

7-(2-cyclopropyl-1H-imidazo[4,5-b]pyridin-6-yl)-4-[5-(cyclopropylmethyl)-2,6-dimethylpyrimidin-4-yl]-2,3,4,5-tetrahydro-1,4-benzoxazepine. Prepared according to the method of example 6 by using 1-methylpropyl 2-cyclopropyl-6-(2,3,4,5-tetrahydro-1,4-benzoxazepin-7-yl)-1H-imidazo[4,5-b]pyridine-1-carboxylate hydrochloride (reagent preparation 19) and 4-chloro-5-(cyclopropylmethyl)-2,6-dimethylpyrimidine (reagent preparation 8) in step 3. ¹H NMR (400 MHz, methanol-d₄) δ 8.71 (s, 1H), 8.25 (s, 1H), 7.78 (d, 1H), 7.58 (dd, 1H), 7.09 (d, 1H), 5.14 (s, 2H), 4.50 (m, 2H), 4.29 (m, 2H), 2.72 (d, 2H), 2.52 (s, 3H), 2.51 (s, 3H), 2.39 (m, 1H), 1.43 (m, 4H), 0.88 (m, 1H), 0.50 (m, 2H), 0.10 (m, 2H); MS (EI) for C₂₈H₃₀N₆O: 467 (MH⁺).

7-(2-cyclopropyl-1H-imidazo[4,5-b]pyridin-6-yl)-4-(2,6,6-trimethyl-5,6,7,8-tetrahydroquinazolin-4-yl)-2,3,4,5-tetrahydro-1,4-benzoxazepine. Prepared according to the method of example 6 by using 1-methylpropyl 2-cyclopropyl-6-(2,3,4,5-tetrahydro-1,4-benzoxazepin-7-yl)-1H-imidazo[4,5-b]pyridine-1-carboxylate hydrochloride (reagent preparation 19) and 4-chloro-2,6,6-trimethyl-5,6,7,8-tetrahydroquinazoline (reagent preparation 8) in step 3. ¹H NMR (400 MHz, methanol-d₄) δ 8.50 (s, 1H), 7.98 (s, 1H), 7.61 (d, 1H), 7.49 (dd, 1H), 7.09 (d, 1H), 4.71 (s, 2H), 4.32 (m, 2H), 3.97 (m, 2H), 2.75 (t, 2H), 2.46 (s, 2H), 2.40 (s, 3H), 2.21 (m, 1H), 1.66 (t, 2H), 1.22 (m, 4H), 0.91 (s, 6H); MS (EI) for C₂₉H₃₂N₆O: 481 (MH⁺).

1-cyclopropyl-N-({6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl)-ethanamine. Prepared according to the method of example 6 by using N-((4-chloro-6,6-dimethyl-5,6,7,8-tetrahydroquinazolin-2-yl)methyl)-1-cyclopropylethanamine (reagent preparation 17) in step 3. ¹H NMR (400 MHz, methanol-d₄) δ 8.52 (s, 1H), 8.05 (s, 1H), 7.63 (d, 1H), 7.50 (dd, 1H), 7.08 (d, 1H), 5.49 (s, 2H), 4.80 (s, 2H), 4.37 (m, 2H), 4.02 (m, 2H), 3.81 (m, 1H), 2.79 (m, 2H), 2.64 (s, 3H), 2.50 (s, 3H), 1.68 (m, 2H), 1.07 (d, 3H), 0.92 (s, 6H), 0.60 (m, 1H), 0.40 (m, 2H), 0.14 (m, 1H); MS (EI) for C₃₂H₃₉N₇O: 538 (MH⁺).

1-{5-(cyclopropylmethyl)-4-methyl-6-[7-(2-methyl-1H-imidazo[4,5-b]pyridin-6-yl)-2,3-dihydro-1,4-benzoxazepin-4(5H)-yl]pyrimidin-2-yl}-N,N-dimethylmethanamine. Prepared according to the method of example 6 by using 1-(4-chloro-5-(cyclopropylmethyl)-6-methylpyrimidin-2-yl)-N,N-dimethylmethanamine (reagent preparation 17) in step 3. ¹H NMR (400 MHz, methanol-d₄) δ 8.52 (s, 1H), 8.05 (s, 1H), 7.64 (d, 1H), 7.49 (dd, 1H), 7.07 (d, 1H), 4.73 (s, 2H), 4.37 (m, 2H), 3.95 (m, 2H), 3.50 (s, 2H), 2.70 (d, 2H), 2.65 (s, 3H), 2.48 (s, 3H), 2.27 (s, 6H), 0.88 (m, 1H), 0.39 (m, 2H), 0.02 (m, 2H); MS (EI) for C₂₈H₃₃N₇O: 538 (MH⁺).

4-(5,6-dimethylpyrimidin-4-yl)-7-(2-methyl-1H-imidazo[4,5-b]pyridin-6-yl)-2,3,4,5-tetrahydro-1,4-benzoxazepine. Prepared according to the method of example 6 by using 4-chloro-5,6-dimethylpyrimidine in step 3. ¹H NMR (400 MHz, methanol-d₄): 8.50 (s, 1H), 8.31 (s, 1H), 8.04 (s, 1H), 7.57 (s, 1H), 7.50 (d, 1H), 7.09 (d, 1H), 4.71 (s, 2H), 4.35 (m, 2H), 3.93 (s, 2H), 2.64 (s, 3H), 2.39 (s, 3H), 2.27 (s, 3H); MS (EI) for C₂₂H₂₂N₆O: 387 (MH⁺).

4-(6-ethyl-5-methylpyrimidin-4-yl)-7-(2-methyl-1H-imidazo[4,5-b]pyridin-6-yl)-2,3,4,5-tetrahydro-1,4-benzoxazepine. Prepared according to the method of example 6 by using 4-chloro-6-ethyl-5-methylpyrimidine (reagent preparation 5) in step 3. ¹H NMR (400 MHz, methanol-d₄): 8.72 (d, 1H), 8.51 (s, 1H), 8.26 (d, 1H), 7.74 (d, 1H), 7.57 (dd, 1H), 7.08 (d, 1H), 5.17 (s, 2H), 4.48 (m, 2H), 4.27 (m, 2H), 2.82 (q, 2H), 2.80 (s, 3H), 2.38 (s, 3H), 1.29 (t, 3H); MS (EI) for C₂₃H₂₄N₆O: 401 (MH⁺).

4-(5-bromo-6-methylpyrimidin-4-yl)-7-(2-methyl-1H-imidazo[4,5-b]pyridin-6-yl)-2,3,4,5-tetrahydro-1,4-benzoxazepine. Prepared according to the method of example 6 by using 5-bromo-4-chloro-6-methylpyrimidine in step 3. ¹H NMR (400 MHz, methanol-d₄): 8.49 (br. s, 1H), 8.33 (s, 1H), 8.02 (br. s, 1H), 7.60 (d, 1H), 7.48 (dd, 1H), 7.08 (d, 1H), 5.05 (s, 2H), 4.36 (m, 2H), 4.16 (m, 2H), 2.64 (s, 3H), 2.55 (s, 3H); MS (EI) for C₂H₁₉BrN₆O: 451/453 (MH⁺).

7-(2-ethyl-1H-imidazo[4,5-b]pyridin-6-yl)-4-(2,6,6-trimethyl-5,6,7,8-tetrahydroquinazolin-4-yl)-2,3,4,5-tetrahydro-1,4-benzoxazepine. Prepared as trifluoroacetate salt according to the method of example 6 by using 2-methylpropyl 2-ethyl-6-(2,3,4,5-tetrahydro-1,4-benzoxazepin-7-yl)-1H-imidazo[4,5-b]pyridine-1-carboxylate hydrochloride (reagent preparation 19) and 4-chloro-6,6-dimethyl-5,6,7,8-tetrahydroquinazolin (reagent preparation 8) in step 3. ¹H NMR (400 MHz, methanol-d₄): 8.67 (d, 1H), 8.19 (d, 1H), 7.76 (s, 1H), 7.57 (d, 1H), 7.09 (d, 1H), 5.13 (s, 2H), 4.45 (m, 2H), 4.31 (m, 2H), 3.09 (m, 2H), 2.81 (m, 2H), 2.57 (s, 2H), 2.50 (s, 3H), 1.68 (m, 2H), 1.48 (m, 3H), 0.94 (s, 6H); MS (EI) for C₂₈H₃₂N₆O: 469 (MH⁺).

N-({6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl)-2-methylpropan-2-amine. Prepared as acetate salt according to the method of example 6 by using N-[(4-chloro-6,6-dimethyl-5,6,7,8-tetrahydroquinazolin-2-yl)methyl]-2-methylpropanamine (reagent preparation 17) in step 3. ¹H NMR (400 MHz, methanol-d₄): 8.52 (d, 1H), 8.06 (d, 1H), 7.63 (d, 1H), 7.52 (dd, 1H), 7.09 (d, 1H), 4.83 (s, 2H), 4.39 (m, 2H), 4.04 (m, 4H), 2.82 (m, 2H), 2.64 (s, 3H), 2.52 (s, 2H), 1.92 (s, 3H), 1.69 (m, 2H), 1.29 (s, 9H), 0.92 (s, 6H); MS (EI) for C₃₁H₃₉N₇O: 526 (MH⁺).

N-({6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl)-2,2,2-trifluoroethanamine. Prepared according to the method of example 6 by using N-[(4-chloro-6,6-dimethyl-5,6,7,8-tetrahydroquinazolin-2-yl)methyl]-2,2,2-triluoroethanamine (reagent preparation 17) in step 3. ¹H NMR (400 MHz, methanol-d₄): 8.53 (s, 1H), 8.06 (s, 1H), 7.65 (d, 1H), 7.50 (dd, 1H), 7.07 (d, 1H), 4.80 (s, 2H), 4.37 (m, 2H), 4.01 (m, 2H), 3.75 (s, 2H), 3.03 (q, 2H), 2.77 (m, 2H), 2.64 (s, 3H), 2.50 (s, 2H), 1.68 (m, 2H), 0.92 (s, 6H); MS (EI) for C₂₉H₃₂F₃N₇O: 552 (MH⁺).

N-({6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl)cyclopentanamine. Prepared as acetate salt according to the method of example 6 by using phenylmethyl[(4-chloro-6,6-dimethyl-5,6,7,8-tetrahydroquinazolin-2-yl)methyl]cyclopentylcarbamate (reagent preparation 17) in step 3 followed by Cbz deprotection. ¹H NMR (400 MHz, methanol-d₄): 8.53 (s, 1H), 8.07 (s, 1H), 7.65 (d, 1H), 7.51 (dd, 1H), 7.07 (d, 1H), 4.84 (s, 2H), 4.36 (m, 2H), 4.03 (m, 2H), 3.81 (s, 2H), 3.13 (m, 1H), 2.78 (m, 2H), 2.64 (s, 3H), 2.51 (s, 2H), 1.89 (s, 3H), 1.76 (m, 2H), 1.68 (m, 2H), 1.54 (m, 2H), 1.43 (m, 2H), 1.25 (m, 2H), 0.92 (s, 6H); MS (EI) for C₃₂H₃₉N₇O: 538 (MH⁺).

N-({6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl)ethanamine. Prepared as acetate salt according to the method of example 6 by using phenylmethyl[(4-chloro-6,6-dimethyl-5,6,7,8-tetrahydroquinazolin-2-yl)methyl](2-fluoroethyl)carbamate (reagent preparation 17) in step 3 followed by Cbz deprotection. ¹H NMR (400 MHz, methanol-d₄): 8.52 (d, 1H), 8.06 (d, 1H), 7.63 (d, 1H), 7.52 (dd, 1H), 7.09 (d, 1H), 4.82 (s, 2H), 4.40 (m, 2H), 4.05 (s, 2H), 4.03 (m, 2H), 2.99 (q, 2H), 2.80 (m, 2H), 2.64 (s, 3H), 2.52 (s, 2H), 1.91 (s, 3H), 1.69 (m, 2H), 1.17 (t, 3H), 0.91 (s, 6H); MS (EI) for C₂₉H₃₅N₇O: 498 (MH⁺).

1-{4,5-dimethyl-6-[7-(2-methyl-3H-imidazo[4,5-b]pyridin-6-yl)-2,3-dihydro-1,4-benzoxazepin-4(5H)-yl]pyrimidin-2-yl}-N,N-dimethylmethanamine. Prepared as triacetate salt according to the method of example 6 by using 1-(4-chloro-5,6-dimethylpyrimidin-2-yl)-N,N-dimethylmethanamine (reagent preparation 17) in step 3. ¹H NMR (400 MHz, methanol-d₄): 8.51 (d, 1H), 8.05 (d, 1H), 7.62 (d, 1H), 7.49 (dd, 1H), 7.06 (d, 1H), 4.78 (s, 2H), 4.36 (m, 2H), 3.97 (m, 2H), 3.79 (s, 2H), 2.64 (s, 3H), 2.49 (s, 6H), 2.41 (s, 3H), 2.27 (s, 3H), 1.91 (s, 9H); MS (EI) for C₂₅H₂₉N₇O: 444 (MH⁺).

(2R)—N-({6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl)butan-2-amine. Prepared as a trifluoroacetate salt according to the method of example 6 by using 2-methylpropyl 2-methyl-6-(2,3,4,5-tetrahydro-1,4-benzoxazepin-7-yl)-1H-imidazo[4,5-b]pyridine-1-carboxylate and (R)-benzyl sec-butyl((4-chloro-6,6-dimethyl-5,6,7,8-tetrahydroquinazolin-2-yl)methyl)carbamate (reagent preparation 17) in step 3 followed by Cbz deprotection. ¹H NMR (400 MHz, DMSO-d6) 8.69 (br s, 3H), 8.24 (br s, 1H), 7.80 (s, 1H), 7.62 (d, 1H), 7.09 (d, 1H), 4.79 (s, 2H), 4.43-4.36 (m, 2H), 4.17-4.11 (m, 2H), 4.00-3.94 (m, 2H), 3.15 (br s, 1H), 2.75 (t, 2H), 2.67 (s, 2H), 2.50 (s, 2H, buried), 1.79-1.66 (m, 1H), 1.62 (t, 2H), 1.46-1.33 (m, 1H), 1.17 (d, 3H), 0.89-0.78 (m, 9H); MS (EI) for C₃₁H₃₉N₇O: 526 (MH⁺).

(2S)—N-({6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl)butan-2-amine. Prepared according to the method of example 6 by using 2-methylpropyl 2-methyl-6-(2,3,4,5-tetrahydro-1,4-benzoxazepin-7-yl)-1H-imidazo[4,5-b]pyridine-1-carboxylate and (S)-benzyl sec-butyl((4-chloro-6,6-dimethyl-5,6,7,8-tetrahydroquinazolin-2-yl)methyl)carbamate (reagent preparation 17) in step 3 followed by Cbz deprotection. ¹H NMR (400 MHz, DMSO-d6) 8.61-8.44 (m, 1H), 8.12-7.93 (m, 1H), 7.71 (d, 1H), 7.54 (dd, 1H), 7.03 (d, 1H), 4.67 (s, 2H), 4.35-4.27 (m, 2H), 3.92-3.84 (m, 2H), 3.56 (q, 2H), 2.69 (t, 3H), 2.54 (s, 2H), 2.45 (s, 3H), 2.42-2.30 (m, 1H), 1.59 (t, 2H), 1.27 (dd, 2H), 1.18-1.04 (m, 1H), 0.87 (s, 6H), 0.83 (d, 3H), 0.70 (t, 3H); MS (EI) for C₃₁H₃₉N₇O: 526 (MH⁺).

1-{4-ethyl-5-methyl-6-[7-(2-methyl-3H-imidazo[4,5-b]pyridin-6-yl)-2,3-dihydro-1,4-benzoxazepin-4(5H)-yl]pyrimidin-2-yl}-N,N-dimethylmethanamine. Prepared according to the method of example 6 by using 2-methylpropyl 2-methyl-6-(2,3,4,5-tetrahydro-1,4-benzoxazepin-7-yl)-1H-imidazo[4,5-b]pyridine-1-carboxylate and 1-(4-chloro-6-ethyl-5-methylpyrimidin-2-yl)-N,N-dimethylmethanamine (reagent preparation 17) in step 3. ¹H NMR (400 MHz, DMSO-d6) δ 8.51 (br s, 1H), 8.03 (br s, 1H), 7.70 (d, 1H), 7.52 (dd, 1H), 7.03 (d, 1H), 4.61 (s, 2H), 4.33-4.28 (m, 2H), 3.84-3.79 (m, 2H), 3.36 (s, 2H), 2.63 (q, 2H), 2.53 (d, 3H), 2.20 (s, 3H), 2.13 (s, 6H), 1.89 (s, 3H), 1.14 (t, 3H); MS (EI) for C₂₆H₃₁N₇O: 458 (MH⁺).

1-{6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methanamine. Prepared as an acetate salt by the method of example 6 using 2-methylpropyl 2-methyl-6-(2,3,4,5-tetrahydro-1,4-benzoxazepin-7-yl)-1H-imidazo[4,5-b]pyridine-1-carboxylate and 2-(azidomethyl)-4-chloro-6,6-dimethyl-5,6,7,8-tetrahydroquinazoline (reagent preparation 45) in step 3 followed by reduction of the azide to the amine (LaRock, R. C. Comprehensive Organic Transformations: A Guide to Functional Group Preparations 1989, VCH Publishers, Inc., New York). ¹H NMR (400 MHz, DMSO-d6) δ 8.52 (d, 1H), 8.04 (d, 1H), 7.74 (d, 1H), 7.54 (dd, 1H), 7.04 (d, 1H), 4.68 (s, 2H), 4.34-4.28 (m, 2H), 3.92-3.87 (m, 2H), 3.63-3.59 (m, 2H), 2.69 (t, 2H), 2.54 (s, 3H), 2.46 (s, 2H), 1.86 (s, 5H), 1.59 (t, 2H), 0.86 (s, 6H); MS (EI) for C₂₇H₃₁N₇O: 470 (MH⁺).

1-{6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}-N-methylmethanamine. Prepared by the method of example 6 using 2-methylpropyl 2-methyl-6-(2,3,4,5-tetrahydro-1,4-benzoxazepin-7-yl)-1H-imidazo[4,5-b]pyridine-1-carboxylate and N-((4-chloro-6,6-dimethyl-5,6,7,8-tetrahydroquinazolin-2-yl)methyl)-N-methyl-2-nitrobenzenesulfonamide (reagent preparation 17) in step 3 followed by 2-nitrobenzenesulfonyl-group deprotection. ¹H NMR (400 MHz, DMSO-d6) δ 8.53 (br s, 1H), 8.06 (br s, 1H), 7.75 (d, 1H), 7.53 (dd, 1H), 7.01 (d, 1H), 4.68 (s, 2H), 4.33-4.27 (m, 2H), 3.91-3.85 (m, 2H), 3.48 (s, 2H), 2.68 (t, 2H), 2.54 (s, 3H), 2.45 (s, 2H), 2.12 (s, 3H), 1.59 (t, 2H), 0.87 (s, 6H); MS (EI) for C₂₈H₃₃N₇O: 484 (MH⁺).

N-({6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl)-2-fluoroethanamine. Prepared by the method of example 6 using 2-methylpropyl 2-methyl-6-(2,3,4,5-tetrahydro-1,4-benzoxazepin-7-yl)-1H-imidazo[4,5-b]pyridine-1-carboxylate and benzyl (4-chloro-6,6-dimethyl-5,6,7,8-tetrahydroquinazolin-2-yl)methyl(2-fluoroethyl)carbamate (reagent preparation 17) in step 3 followed by Cbz deprotection. ¹H NMR (400 MHz, DMSO-d6) δ 12.83 (s, 0.5H), 12.55 (s, 0.5H), 8.57 (d, 0.5H), 8.49 (d, 0.5H), 8.09 (d, 0.5H), 7.97 (d, 0.5H), 7.73 (d, 1H), 7.54 (dd, 1H), 7.03 (dd, 1H), 4.68 (s, 2H), 4.45 (t, 1H), 4.38-4.25 (m, 3H), 3.93-3.82 (m, 2H), 3.66 (s, 2H), 2.85-2.65 (m, 4H), 2.57-2.52 (m, 3H), 2.46 (s, 2H), 1.59 (t, 2H), 0.86 (s, 6H); MS (EI) for C₂₉H₃₄FN₇O: 516 (MH⁺).

N-({6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl)methanesulfonamide. Prepared by the method of example 6 using 2-methylpropyl 2-methyl-6-(2,3,4,5-tetrahydro-1,4-benzoxazepin-7-yl)-1H-imidazo[4,5-b]pyridine-1-carboxylate and N-((4-chloro-6,6-dimethyl-5,6,7,8-tetrahydroquinazolin-2-yl)methyl)methanesulfonamide (reagent preparation 17) in step 3. ¹H NMR (400 MHz, DMSO-d6) δ 8.52 (br s, 1H), 8.05 (br s, 1H), 7.74 (d, 1H), 7.56 (dd, 1H), 7.27 (t, 1H), 7.06 (d, 1H), 4.68 (s, 2H), 4.37-4.31 (m, 2H), 4.12 (d, 2H), 3.93-3.87 (m, 2H), 2.85 (s, 3H), 2.71 (t, 2H), 2.54 (s, 3H), 2.47 (s, 2H), 1.60 (t, 2H), 0.85 (s, 6H); MS (EI) for C₂₈H₃₃N₇O₃S: 548 (MH⁺).

1-{5-ethyl-4-methyl-6-[7-(2-methyl-3H-imidazo[4,5-b]pyridin-6-yl)-2,3-dihydro-1,4-benzoxazepin-4(5H)-yl]pyrimidin-2-yl}-N,N-dimethylmethanamine. Prepared as a diacetate salt by the method of example 6 using 2-methylpropyl 2-methyl-6-(2,3,4,5-tetrahydro-1,4-benzoxazepin-7-yl)-1H-imidazo[4,5-b]pyridine-1-carboxylate and 1-(4-chloro-5-ethyl-6-methylpyrimidin-2-yl)-N,N-dimethylmethanamine (reagent preparation 17) in step 3. ¹H NMR (400 MHz, DMSO-d6) δ 8.51 (br s, 1H), 8.03 (br s, 1H), 7.67 (d, 1H), 7.52 (dd, 1H), 7.02 (d, 1H), 4.63 (s, 2H), 4.35-4.30 (m, 2H), 3.83-3.78 (m, 2H), 3.32 (s, 2H), 2.65-2.57 (m, 1H), 2.54 (s, 3H), 2.52-2.45 (m, 1H (buried)), 2.36 (s, 3H), 2.10 (s, 6H), 1.86 (s, 8H), 1.15 (t, 3H); MS (EI) for C₂H₃₁N₇O: 458 (MH⁺).

1-{4-[7-(2-cyclopropyl-1H-imidazo[4,5-b]pyridin-6-yl)-2,3-dihydro-1,4-benzoxazepin-4(5H)-yl]-6,6-dimethyl-5,6,7,8-tetrahydroquinazolin-2-yl}-N,N-dimethylmethanamine. Prepared as acetate according to the method of example 6 by using 2-methylpropyl 2-cyclopropyl-6-(2,3,4,5-tetrahydro-1,4-benzoxazepin-7-yl)-1H-imidazo[4,5-b]pyridine-1-carboxylate (reagent preparation 19) in step 1 and 1-(4-chloro-6,6-dimethyl-5,6,7,8-tetrahydroquinazolin-2-yl)-N,N-dimethylmethanamine (reagent preparation 17) in step 3. ¹H NMR (400 MHz, d₆-DMSO): 8.49 (s, 1H), 7.98 (s, 1H), 7.68 (d, 1H), 7.51 (dd, 1H), 7.01 (d, 1H), 4.63 (s, 2H), 4.30 (m, 2H), 3.85 (m, 2H), 2.68 (m, 2H), 2.44 (s, 2H), 2.14 (m, 1H), 2.10 (s, 6H), 1.88 (s, 2H), 1.59 (m, 2H), 1.10 (m, 4H), 0.85 (s, 6H). MS (EI) for C₃₁H₃₇N₇O: 524 (MH⁺).

4-[6,6-dimethyl-2-({[2-(methyloxy)ethyl]oxy}methyl)-5,6,7,8-tetrahydroquinazolin-4-yl]-7-(2-methyl-1H-imidazo[4,5-b]pyridin-6-yl)-2,3,4,5-tetrahydro-1,4-benzoxazepine. Prepared according to the method of example 6 by using 4-chloro-6,6-dimethyl-2-({[2-(methyloxy)ethyl]oxy}methyl)-5,6,7,8-tetrahydroquinazoline (reagent preparation 17) in step 3. MS (EI) for C₃₀H₃₆N₆O₃: 529 (MH⁺).

N-({6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl)-2-(methyloxy)ethanamine. Prepared according to the method of example 6 by using N-[(4-chloro-6,6-dimethyl-5,6,7,8-tetrahydroquinazolin-2-yl)methyl]-2-(methyloxy)ethanamine (reagent preparation 17) in step 3. ¹H NMR (400 MHz, d₆-DMSO): 9.50 (brs, 1H), 8.89 (s, 1H), 8.46 (s, 1H), 8.06 (brs, 1H), 7.69 (dd, 1H), 7.08 (d, 1H), 5.08 (brs, 1H), 4.48 (s, 2H), 4.26 (s, 2H), 4.12 (m, 2H), 3.54 (m, 1H), 3.20 (s, 3H), 3.12 (m, 2H), 2.84 (s, 3H), 2.76 (m, 2H), 2.54 (s, 2H), 2.51 (s, 3H), 1.62 (m, 2H), 0.84 (s, 6H). MS (EI) for C₃₀H₃₇N₇O₂: 527 (MH⁺).

4-[(7S)-7-ethyl-5,6,7,8-tetrahydroquinazolin-4-yl]-7-(2-methyl-1H-imidazo[4,5-b]pyridin-6-yl)-2,3,4,5-tetrahydro-1,4-benzoxazepine. Prepared according to the method of example 6 by using (7S)-4-chloro-7-ethyl-5,6,7,8-tetrahydroquinazoline (reagent preparation 17) in step 3. ¹H NMR (400 MHz, Methanol-d₄): 8.49 (br, 1H), 8.32 (s, 1H), 8.04 (br, 1H), 7.57 (s, 1H), 7.46 (d, 1H), 7.08 (d, 1H), 4.74 (b, 2H), 4.42 (m, 1H), 4.27 (m, 1H), 4.08 to 3.89 (m, 2H), 2.94 to 2.80 (m, 2H), 2.65 (s, 3H), 2.60 (m, 1H), 2.29 (m, 1H), 1.97 (m, 1H), 1.72 (m, 1H), 1.40 (m, 2H), 1.11 (m, 1H), 0.97 (t, 3H); MS (EI) for C₂₆H₂₈N₆O: 441 (MH⁺).

4-[(7S)-7-ethyl-2-methyl-5,6,7,8-tetrahydroquinazolin-4-yl]-7-(2-methyl-1H-imidazo[4,5-b]pyridin-6-yl)-2,3,4,5-tetrahydro-1,4-benzoxazepine. Prepared according to the method of example 6 by using (7S)-4-chloro-7-ethyl-2-methyl-5,6,7,8-tetrahydroquinazoline (reagent preparation 8) in step 3. ¹H NMR (400 MHz, Methanol-d₄): 8.39 (s, 1H), 7.95 (s, 1H), 7.52 (s, 1H), 7.31 (d, 1H), 6.98 (d, 1H), 4.75 (b, 2H), 4.43 (m, 1H), 4.12 (m, 1H), 4.00 (m, 1H), 3.88 (m, 1H), 2.82 to 2.67 (m, 2H), 2.54 (s, 3H), 2.49 (m, 1H), 2.28 (s, 3H), 2.19 (m, 1H), 1.86 (m, 1H), 1.63 (m, 1H), 1.31 (m, 2H), 1.05 (m, 1H), 0.91 (t, 3H); MS (EI) for C₂₇H₃₀N₆O: 455 (MH⁺).

4-[6,6-dimethyl-2-(pyrrolidin-1-ylmethyl)-5,6,7,8-tetrahydroquinazolin-4-yl]-7-(2-methyl-3H-imidazo[4,5-b]pyridin-6-yl)-2,3,4,5-tetrahydro-1,4-benzoxazepine. Prepared as the trifluoroacetic acid salt according to the method of example 6 by using 4-chloro-6,6-dimethyl-2-(pyrrolidin-1-ylmethyl)-5,6,7,8-tetrahydroquinazoline (reagent preparation 17) in step 3. ¹H NMR (400 MHz, Methanol-d₄): 8.95 (br, 1H), 8.44 (br, 1H), 7.85 (s, 1H), 7.59 (s, 1H), 7.48 (d, 1H), 7.12 (d, 1H), 5.09 (s, 2H), 4.56 (s, 2H), 4.54 (m, 2H), 4.22 (m, 2H), 3.38 (m, 4H), 2.85 (t, 2H), 2.85 (s, 3H), 2.56 (s, 2H), 1.93 (m, 4H), 1.68 (t, 2H), 0.90 (s, 6H); MS (EI) for C₃₁H₃₇N₇O: 524 (MH⁺).

1-{6,6-dimethyl-4-[7-(2-methyl-3H-imidazo[4,5-b]pyridin-6-yl)-2,3-dihydro-1,4-benzoxazepin-4(5H)-yl]-5,6,7,8-tetrahydroquinazolin-2-yl}-N,N-dimethylethanamine. Prepared according to the method of example 6 by using 1-(4-chloro-6,6-dimethyl-5,6,7,8-tetrahydroquinazolin-2-yl)-N,N-dimethylethanamine (reagent preparation 46) in step 3. ¹H NMR (400 MHz, Methanol-d₄): 8.52 (s, 1H), 8.04 (s, 1H), 7.63 (s, 1H), 7.54 (d, 1H), 7.03 (d, 1H), 5.03 (s, 2H), 4.78 (m, 2H), 3.99 (m, 2H), 3.52 (s, 1H), 2.77 (t, 2H), 2.63 (s, 3H), 2.49 (s, 2H), 2.13 (s, 6H), 1.66 (t, 2H), 1.28 (d, 3H), 0.91 (s, 6H); MS (EI) for C₃₀H₃₇N₇O: 512 (MH⁺).

4-[6,6-dimethyl-2-(1-pyrrolidin-1-ylethyl)-5,6,7,8-tetrahydroquinazolin-4-yl]-7-(2-methyl-3H-imidazo[4,5-b]pyridin-6-yl)-2,3,4,5-tetrahydro-1,4-benzoxazepine. Prepared according to the method of example 6 by using 4-chloro-6,6-dimethyl-2-(1-pyrrolidin-1-ylethyl)-5,6,7,8-tetrahydroquinazoline (reagent preparation 46) in step 3. ¹H NMR (400 MHz, Methanol-d₄): 8.54 (s, 1H), 8.05 (s, 1H), 7.65 (s, 1H), 7.48 (d, 1H), 7.05 (d, 1H), 4.77 (s, 2H), 4.37 (m, 2H), 4.02 (m, 2H), 3.57 (s, 1H), 2.77 (t, 2H), 2.67 (m, 2H), 2.63 (s, 3H), 2.50 (s, 2H), 2.47 (m, 2H), 1.69 to 1.64 (m, 6H), 1.37 (d, 3H), 0.90 (s, 6H); MS (EI) for C₃₂H₃₉N₇O: 538 (MH⁺).

Phenylmethyl (2S)-2-{6,6-dimethyl-4-[7-(2-methyl-3H-imidazo[4,5-b]pyridin-6-yl)-2,3-dihydro-1,4-benzoxazepin-4(5H)-yl]-5,6,7,8-tetrahydroquinazolin-2-yl}pyrrolidine-1-carboxylate. Prepared according to the method of example 6 by using phenylmethyl (2S)-2-(4-chloro-6,6-dimethyl-5,6,7,8-tetrahydroquinazolin-2-yl)pyrrolidine-1-carboxylate (reagent preparation 18) in step 3. ¹H NMR (400 MHz, Methanol-d₄): 8.47 (s, 0.35H), 8.43 (s, 0.65H), 7.99 (s, 0.35H), 7.95 (0.65H), 7.48 (br, 1H), 7.45 to 7.38 (m, 2H), 7.32 (br, 1H), 7.08 to 7.01 (m, 3H), 6.72 (dd, 1H), 5.03 (dd, 2H), 4.77 (s, 0.35H), 4.73 (s, 0.65H), 4.69 to 4.64 (m, 3H), 4.44 to 4.21 (m, 2H), 4.01 to 3.77 (m, 2H), 3.53 to 3.58 (m, 2H), 2.78 to 1.68 (m, 2H), 2.63 (s, 3H), 2.51 (d, 2H), 2.25 (m, 1H), 1.76 (m, 1H), 1.64 (m, 2H), 0.98 to 0.83 (m, 6H); MS (EI) for C₃₈H₄₁N₇O₃: 644 (MH⁺).

4-{6,6-dimethyl-2-[(2S)-pyrrolidin-2-yl]-5,6,7,8-tetrahydroquinazolin-4-yl}-7-(2-methyl-3H-imidazo[4,5-b]pyridin-6-yl)-2,3,4,5-tetrahydro-1,4-benzoxazepine. Prepared according to the method of example 6 by using phenylmethyl (2S)-2-(4-chloro-6,6-dimethyl-5,6,7,8-tetrahydroquinazolin-2-yl)pyrrolidine-1-carboxylate (reagent preparation 18) in step 3 followed by Cbz group deprotection. ¹H NMR (400 MHz, Methanol-d₄): 8.53 (s, 1H), 8.07 (s, 1H), 7.67 (s, 1H), 7.52 (d, 1H), 7.08 (d, 1H), 4.84 (s, 2H), 4.50 (m, 1H), 4.38 (m, 2H), 4.02 (m, 2H), 3.27 to 3.10 (m, 3H), 2.82 (t, 2H), 2.66 (s, 3H), 2.51 (s, 2H), 2.33 (m, 1H), 1.99 (m, 1H), 1.81 (m, 1H), 1.68 (t, 2H), 0.90 (d, 6H); MS (EI) for C₃₀H₃₅N₇O: 510 (MH⁺).

4-{6,6-dimethyl-2-[(2R)-pyrrolidin-2-yl]-5,6,7,8-tetrahydroquinazolin-4-yl}-7-(2-methyl-3H-imidazo[4,5-b]pyridin-6-yl)-2,3,4,5-tetrahydro-1,4-benzoxazepine. Prepared according to the method of example 6 by using phenylmethyl (2R)-2-(4-chloro-6,6-dimethyl-5,6,7,8-tetrahydroquinazolin-2-yl)pyrrolidine-1-carboxylat (reagent preparation 18) in step 3 followed by Cbz group deprotection. ¹H NMR (400 MHz, Methanol-d₄): 8.55 (s, 1H), 8.09 (s, 1H), 7.67 (s, 1H), 7.52 (d, 1H), 7.07 (d, 1H), 4.85 (s, 2H), 4.49 (m, 1H), 4.38 (m, 2H), 4.01 (m, 2H), 3.28 to 3.10 (m, 3H), 2.79 (t, 2H), 2.63 (s, 3H), 2.52 (s, 2H), 2.32 (m, 1H), 1.99 (m, 1H), 1.79 (m, 1H), 1.67 (t, 2H), 0.91 (d, 6H); MS (EI) for C₃₀H₃₅N₇O: 510 (MH⁺).

4-(6,6-dimethyl-2-pyrrolidin-2-yl-5,6,7,8-tetrahydroquinazolin-4-yl)-7-(2-methyl-3H-imidazo[4,5-b]pyridin-6-yl)-2,3,4,5-tetrahydro-1,4-benzoxazepine. Prepared according to the method of example 6 by using phenylmethyl 2-(4-chloro-6,6-dimethyl-5,6,7,8-tetrahydroquinazolin-2-yl)pyrrolidine-1-carboxylate (reagent preparation 18) in step 3 followed by Cbz group deprotection. ¹H NMR (400 MHz, Methanol-d₄): 8.53 (s, 1H), 8.08 (s, 1H), 7.67 (s, 1H), 7.51 (d, 1H), 7.04 (d, 1H), 4.83 (s, 2H), 4.37 (m, 3H), 4.02 (m, 2H), 3.27 (m, 1H), 3.17 to 3.01 (m, 2H), 2.80 (t, 2H), 2.64 (s, 3H), 2.51 (s, 2H), 2.28 (m, 1H), 1.90 (m, 1H), 1.76 (m, 1H), 1.69 (t, 2H), 0.91 (d, 6H); MS (EI) for C₃₀H₃₅N₇O: 510 (MH⁺).

4-{6,6-dimethyl-2-[(methyloxy)methyl]-5,6,7,8-tetrahydroquinazolin-4-yl}-7-(2-methyl-3H-imidazo[4,5-b]pyridin-6-yl)-2,3,4,5-tetrahydro-1,4-benzoxazepine. Prepared according to the method of example 6 by using 4-chloro-6,6-dimethyl-2-[(methyloxy)methyl]-5,6,7,8-tetrahydroquinazoline (reagent preparation 17) in step 3. ¹H NMR (400 MHz, Methanol-d₄): 8.55 (br, 1H), 8.07 (br, 1H), 7.66 (s, 1H), 7.50 (d, 1H), 7.08 (d, 1H), 4.76 (s, 2H), 4.37 (s, 2H), 4.32 (m, 2H), 4.01 (m, 2H), 3.33 (s, 3H), 2.77 (t, 2H), 2.63 (s, 3H), 2.49 (s, 2H), 1.67 (t, 2H), 0.91 (s, 6H); MS (EI) for C₂H₃₂N₆O₂: 485 (MH⁺).

4-(2-ethenyl-6,6-dimethyl-5,6,7,8-tetrahydroquinazolin-4-yl)-7-(2-methyl-3H-imidazo[4,5-b]pyridin-6-yl)-2,3,4,5-tetrahydro-1,4-benzoxazepine. Prepared according to the method of example 6 by using 4-[6,6-dimethyl-2-(2-pyrrolidin-1-ylethyl)-5,6,7,8-tetrahydroquinazolin-4-yl]-7-(2-methyl-3H-imidazo[4,5-b]pyridin-6-yl)-2,3,4,5-tetrahydro-1,4-benzoxazepine in step 3. ¹H NMR (400 MHz, Methanol-d₄): 8.51 (s, 1H), 8.07 (s, 1H), 7.63 (s, 1H), 7.49 (d, 1H), 7.06 (d, 1H), 6.59 (dd, 1H), 6.40 (d, 1H), 5.51 (d, 1H), 4.77 (2H), 4.37 (m, 2H), 4.00 (m, 2H), 2.77 (t, 2H), 2.63 (s, 3H), 2.51 (s, 2H), 1.67 (t, 2H), 0.92 (s, 6H); MS (EI) for C₂₈H₃₀N₆O: 485 (MH⁺).

4-[6,6-dimethyl-2-(2-pyrrolidin-1-ylethyl)-5,6,7,8-tetrahydroquinazolin-4-yl]-7-(2-methyl-3H-imidazo[4,5-b]pyridin-6-yl)-2,3,4,5-tetrahydro-1,4-benzoxazepine. Prepared according to the method of example 6 by using 4-chloro-6,6-dimethyl-2-(2-pyrrolidin-1-ylethyl)-5,6,7,8-tetrahydroquinazoline (reagent preparation 8) in step 3. ¹H NMR (400 MHz, Methanol-4₄): 8.56 (s, 1H), 8.11 (s, 1H), 7.68 (s, 1H), 7.52 (d, 1H), 7.06 (d, 1H), 4.81 (2H), 4.37 (m, 2H), 4.00 (m, 2H), 3.34 (m, 2H), 2.99 (m, 6H), 2.75 (t, 2H), 2.64 (s, 3H), 2.49 (s, 2H), 1.95 (m, 4H), 1.67 (t, 2H), 0.92 (s, 6H); MS (EI) for C₃₂H₃₉N₇O: 538 (MH⁺).

7-(2-methyl-1H-imidazo[4,5-b]pyridin-6-yl)-4-(5-methylpyrimidin-4-yl)-2,3,4,5-tetrahydro-1,4-benzoxazepine. Synthesized according to the method of example 6 using 4-chloro-5-methylpyrimidine in step 3. ¹H NMR (400 MHz, d₆-DMSO): 8.89 (d, 1H), 8.80 (s, 1H), 8.45 (d, 1H), 8.28 (s, 1H), 7.91 (d, 1H), 7.69 (dd, 1H), 7.10 (d, 1H), 5.27 (s, 2H), 4.50 (m, 2H), 4.33 (m, 2H), 2.84 (s, 3H), 2.43 (s, 3H), 1.76 (s, 3H); MS (EI) for C₂₁H₂₀N₆O: 373 (MH⁺).

4-[2,6-dimethyl-5-(1-methylethyl)pyrimidin-4-yl]-7-(2-methyl-1H-imidazo[4,5-b]pyridin-6-yl)-2,3,4,5-tetrahydro-1,4-benzoxazepine. Synthesized according to the method of example 6 using 4-chloro-5-isopropyl-2,6-dimethylpyrimidine (reagent preparation 8) in step 3. ¹H NMR (400 MHz, d₆-DMSO): 8.74 (s, 1H), 8.29 (s, 1H), 7.82 (s, 1H), 7.63 (dd, 1H), 7.30 (s, 1H), 7.17 (s, 1H), 7.04 (s, 1H), 5.06 (s, 2H), 4.50 (m, 2H), 4.03 (m, 2H), 3.08 (m, 1H) 2.71 (s, 3H), 2.54 (s, 3H), 2.41 (s, 3H), 1.32 (d, 6H); MS (EI) for C₂₅H₂₈N₆O: 429 (MH⁺).

7-(2-methyl-1H-imidazo[4,5-b]pyridin-6-yl)-4-[6-methyl-5-(1-methylethyl)pyrimidin-4-yl]-2,3,4,5-tetrahydro-1,4-benzoxazepine. Synthesized according to the method of example 6 using 4-chloro-5-isopropyl-6-methylpyrimidine (reagent preparation 5) in step 3. ¹H NMR (400 MHz, d₆-DMSO): 8.81 (s, 1H), 8.64 (s, 1H), 8.36 (s, 1H), 7.65 (d, 1H), 7.03 (d, 1H), 5.04 (s, 2H), 4.53 (s, 2H), 4.02 (s, 2H), 3.11 (m, 1H), 2.76 (s, 3H), 2.56 (s, 3H), 1.34 (d, 6H); MS (EI) for C₂₄H₂₆N₆O: 415 (MH⁺).

7-(2-methyl-1H-imidazo[4,5-b]pyridin-6-yl)-4-(7-methyl-7-phenyl-5,6,7,8-tetrahydroquinazolin-4-yl)-2,3,4,5-tetrahydro-1,4-benzoxazepine. Synthesized according to the method of example 6 using 4-chloro-7-methyl-7-phenyl-5,6,7,8-tetrahydroquinazoline (reagent preparation 3) in step 3. ¹H NMR (400 MHz, d₆-DMSO): 8.49 (s, 1H), 8.37 (s, 1H), 7.92 (d, 1H), 7.48 (d, 1H), 7.31 (s, 1H), 7.29 (s, 1H), 7.22 (d, 1H), 7.13 (m, 2H), 7.02 (m, 1H), 6.94-6.80 (m, 1H), 4.42-4.56 (m, 2H), 4.32 (m, 2H), 3.94 (d, 1H), 3.76 (m, 1H), 3.14 (d, 1H), 2.81 (d, 1H), 2.68 (d, 1H), 2.55 (d, 3H), 2.55 (d, 3H), 2.29 (m, 1H), 2.09 (m, 1H), 1.84 (m, 1H), 1.32 (s, 3H); MS (EI) for C₃₁H₃₀N₆O: 503 (MH⁺).

N,N,2-trimethyl-4-[7-(2-methyl-1H-imidazo[4,5-b]pyridin-6-yl)-2,3-dihydro-1,4-benzoxazepin-4(5H)-yl]pyrimidine-5-sulfonamide. Synthesized according to the method of example 6 using 4-chloro-N,N,2-trimethylpyrimidine-5-sulfonamide in step 3. ¹H NMR (400 MHz, d₆-DMSO): 8.51 (s, 1H), 8.03 (s, 1H), 7.94 (s, 1H), 7.52 (dd, 1H) 7.04 (d, 1H), 6.71 (d, 1H), 5.05 (s, 2H), 4.31 (d, 2H), 4.24 (d, 2H), 2.84 (s, 6H), 2.54 (s, 3H), 2.40 (s, 3H); MS (EI) for C₂₃H₂₅N₇O₃S: 480 (MH⁺).

7-(2-methyl-1H-imidazo[4,5-b]pyridin-6-yl)-4-[2-methyl-5-(morpholin-4-ylsulfonyl)pyrimidin-4-yl]-2,3,4,5-tetrahydro-1,4-benzoxazepine. Synthesized according to the method of example 6 using 4-(4-chloro-2-methylpyrimidin-5-ylsulfonyl)morpholine in step 3. ¹H NMR (400 MHz, d₆-DMSO): 8.58 (s, 1H), 8.50 (d, 1H), 8.03 (d, 1H), 7.81 (s, 1H), 7.53 (d, 1H), 7.03 (d, 1H), 5.04 (s, 2H), 4.29 (s, 2H), 3.61 (s, 2H), 3.12 (s, 2H), 2.42 (s, 3H); MS (EI) for C₂₅H₂₇N₇O₄S: 522 (MH⁺).

7-(2-cyclopropyl-1H-imidazo[4,5-b]pyridin-6-yl)-4-(6,6-dimethyl-5,6-dihydroquinazolin-4-yl)-2,3,4,5-tetrahydro-1,4-benzoxazepine. Synthesized according to the method of example 6 using isobutyl 6-bromo-2-cyclopropyl-1H-imidazo[4,5-b]pyridine-1-carboxylate (reagent preparation 19) in step 1 and 4-chloro-6,6-dimethyl-5,6-dihydroquinazoline (reagent preparation 3) in step 3. ¹H NMR (400 MHz, d₆-DMSO): 8.64 (s, 1H), 8.61 (s, 1H), 8.14 (m, 1H), 7.77 (d, 1H), 7.59 (dd, 1H), 7.04 (d, 1H), 6.47 (d, 1H), 6.34 (d, 1H), 5.00 (s, 2H), 4.46 (m, 2H), 4.11 (s, 2H), 2.83 (s, 2H), 2.24 (m, 1H), 1.24-1.18 (m, 5H), 0.99 (s, 6H); MS (EI) for C₂₈H₂₈N₆O: 465 (MH⁺).

4-(6,6-dimethyl-5,6-dihydroquinazolin-4-yl)-7-(2-methyl-1H-imidazo[4,5-b]pyridin-6-yl)-2,3,4,5-tetrahydro-1,4-benzoxazepine. Synthesized according to the method of example 6 using 4-chloro-6,6-dimethyl-5,6-dihydroquinazoline (reagent preparation 3) in step 3. ¹H NMR (400 MHz, d₆-DMSO): 8.64 (s, 1H), 8.59 (s, 1H), 8.18 (s, 1H), 7.77 (d, 1H), 7.60 (dd, 1H), 7.05 (d, 1H), 6.43 (d, 1H), 6.32 (d, 1H), 4.94 (s, 2H), 4.43 (m, 2H), 4.06 (br s, 2H), 2.82 (s, 2H), 2.62 (s, 3H), 0.99 (s, 6H); MS (EI) for C₂₆H₂₆N₆O: 439 (MH⁺).

7-(2-methyl-1H-imidazo[4,5-b]pyridin-6-yl)-4-(6,6,8-trimethyl-5,6-dihydroquinazolin-4-yl)-2,3,4,5-tetrahydro-1,4-benzoxazepine. Synthesized according to the method of example 6 using 4-chloro-6,6,8-trimethyl-5,6-dihydroquinazoline (reagent preparation 3) in step 3. ¹H NMR (400 MHz, d₆-DMSO): 8.74 (d, 1H), 8.54 (s, 1H), 8.30 (d, 1H), 7.77 (d, 1H), 7.61 (dd, 1H), 7.05 (d, 1H), 6.09 (s, 1H), 4.82 (s, 2H), 4.42 (m, 2H), 3.96 (br s, 2H), 2.73 (s, 2H), 2.69 (s, 3H), 1.96 (d, 3H), 0.92 (s, 6H); MS (EI) for C₂₇H₂₆N₆O: 453 (MH⁺).

7-(2-methyl-1H-imidazo[4,5-b]pyridin-6-yl)-4-(6,6,7,8-tetramethyl-5,6-dihydroquinazolin-4-yl)-2,3,4,5-tetrahydro-1,4-benzoxazepine. Synthesized according to the method of example 6 using 4-chloro-6,6,7,8-tetramethyl-5,6-dihydroquinazoline (reagent preparation 3) in step 3. ¹H NMR (400 MHz, d₆-DMSO): 8.69 (s, 1H), 8.53 (s, 1H), 8.24 (s, 1H), 7.77 (d, 1H), 7.61 (dd, 1H), 7.06 (d, 1H), 4.86 (s, 2H), 4.42 (m, 2H), 4.00 (br s, 2H), 2.71 (s, 2H), 2.66 (s, 3H), 1.97 (s, 3H), 1.87 (s, 3H), 0.88 (s, 6H); MS (EI) for C₂₈H₃₀N₆O: 467 (MH⁺).

N,N-dimethyl-1-{4-[7-(2-methyl-1H-imidazo[4,5-b]pyridin-6-yl)-2,3-dihydro-1,4-benzoxazepin-4(5H)-yl]-7-(methyloxy)quinazolin-2-yl}methanamine. Synthesized according to the method of example 6 using 1-(4-chloro-7-methoxyquinazolin-2-yl)-N,N-dimethylmethanamine (reagent preparation 20) in step 3. ¹H NMR (400 MHz, d₆-DMSO): 8.59-8.50 (br d, 1H), 8.11-7.98 (br d, 1H), 7.94 (d, 1H), 7.76 (d, 1H), 7.53 (dd, 1H), 7.16 (d, 1H), 7.08 (dd, 1H), 6.99 (d, 1H), 5.06 (s, 2H), 4.46 (s, 2H), 4.21 (m, 2H), 3.89 (s, 3H), 3.45 (s, 2H), 2.52 (s, 3H), 2.13 (s, 6H), 1.91 (s, 2H); MS (EI) for C₂₈H₂₉N₇O₂: 496 (MH⁺).

1-{6-fluoro-4-[7-(2-methyl-1H-imidazo[4,5-b]pyridin-6-yl)-2,3-dihydro-1,4-benzoxazepin-4(5H)-yl]quinazolin-2-yl}-N,N-dimethylmethanamine. Synthesized according to the method of example 6 using 1-(4-chloro-6-fluoroquinazolin-2-yl)-N,N-dimethylmethanamine (reagent preparation 20) in step 3. ¹H NMR (400 MHz, d₆-DMSO): 8.63-8.54 (br d, 1H), 8.15-8.03 (br d, 1H), 7.90-7.75 (m, 4H), 7.58 (dd, 1H), 7.02 (d, 1H), 5.16 (s, 1H), 4.54 (br m, 2H), 4.27 (br m, 2H), 4.00 (br s, 2H), 3.34 (s, 6H), 2.54 (s, 3H); MS (EI) for C₂₇H₂₆FN₇O: 484 (MH⁺).

4-{2-[(3,3-difluoropyrrolidin-1-yl)methyl]-6,6-dimethyl-5,6,7,8-tetrahydroquinazolin-4-yl}-7-(2-methyl-1H-imidazo[4,5-b]pyridin-6-yl)-2,3,4,5-tetrahydro-1,4-benzoxazepine. Synthesized according to the method of example 6 using 2-((3,3-difluoropyrrolidin-1-yl)methyl)-4,6,6-trimethyl-5,6,7,8-tetrahydroquinazoline (reagent preparation 17) in step 3. ¹H NMR (400 MHz, d₆-DMSO): 8.88 (d, 1H), 8.44 (d, 1H), 7.99 (s, 1H), 7.67 (dd, 1H), 7.05 (d, 1H), 5.08 (s, 1H), 4.49 (br m, 2H), 4.15 (br m, 2H), 4.10 (br s, 2H), 3.24 (br t, 2H), 3.10 (br m, 2), 2.81 (m, 4H), 2.54 (s, 2H), 2.31-2.23 (m, 2H), 1.59 (t, 2H), 0.87 (s, 6H); MS (EI) for C₃₁H₃₅F₂N₇O: 560 (MH⁺).

4′-[7-(2-methyl-3H-imidazo[4,5-b]pyridin-6-yl)-2,3-dihydro-1,4-benzoxazepin-4(5H)-yl]-7′,8′-dihydro-5′H-spiro[cyclopropane-1,6′-quinazoline]. The dihydrochloride salt was prepared as in example 6 using 4′-chloro-7′,8′-dihydro-5′H-spiro[cyclopropane-1,6′-quinazoline](reagent example 3) in step 3. ¹H NMR (400 MHz, CD₃OD) δ 8.87 (d, 1H), 8.53 (s, 1H), 8.41 (d, 1H), 7.80 (d, 1H), 7.61 (dd, 1H), 7.11 (d, 1H), 5.19 (s, 2H), 4.48 (t, 2H), 4.31 (t, 2H), 2.93 (t, 2H), 2.91 (s, 3H), 2.72 (bs, 2H), 1.75 (t, 3H), 0.52-0.34 (m, 4H); MS (ES) for C₂₆H₂₆N₆O: 439.2 (MH⁺).

4-(7,7-dimethyl-5,6,7,8-tetrahydroquinazolin-4-yl)-7-(2-methyl-1H-imidazo[4,5-b]pyridin-6-yl)-2,3,4,5-tetrahydro-1,4-benzoxazepine. The dihydrochloride was prepared as in example 6 using 4-chloro-7,7-dimethyl-5,6,7,8-tetrahydroquinazoline (reagent preparation 3) in step 3. ¹H NMR (400 MHz, CD₃OD) δ 8.85 (t, 1H), 8.54 (s, 1H), 8.41 (t, 1H), 7.82 (d, 1H), 7.66-7.55 (m, 1H), 7.12 (d, 1H), 5.25 (s, 2H), 4.54-4.46 (m, 2H), 4.45-4.37 (m, 2H), 2.91 (s, 3H), 2.88 (t, 2H), 2.60 (s, 2H), 1.60 (t, 2H), 1.10 (s, 6H); MS (ES) for C₂₆H₂₈N₆O: 441.2 (MH⁺).

7-(2-methyl-1H-imidazo[4,5-b]pyridin-6-yl)-4-(6-methyl-5,6,7,8-tetrahydroquinazolin-4-yl)-2,3,4,5-tetrahydro-1,4-benzoxazepine. The dihydrochloride salt was prepared as in example 6 using 4-chloro-6-methyl-5,6,7,8-tetrahydroquinazoline (reagent preparation 3) in step 3. ¹H NMR (400 MHz, CD₃OD) δ 8.87 (d, 1H), 8.53 (s, 1H), 8.43 (d, 1H), 7.81 (d, 1H), 7.61 (dd, 1H), 7.10 (d, 1H), 5.20 (dd, 2H), 4.70-4.47 (m, 2H), 4.42-4.10 (m, 2H), 2.91 (s, 3H), 2.94-2.86 (m, 2H), 2.71-2.58 (m, 2H), 2.02-1.91 (m, 1H), 1.74-1.48 (m, 2H), 1.08 (d, 3H) MS (ES) for C₂₅H₂₆N₆O: 427.2 (MH⁺).

4-(6-ethyl-5,6,7,8-tetrahydroquinazolin-4-yl)-7-(2-methyl-1H-imidazo[4,5-b]pyridin-6-yl)-2,3,4,5-tetrahydro-1,4-benzoxazepine. The dihydrochloride was prepared as in example 6 using 4-chloro-6-ethyl-5,6,7,8-tetrahydroquinazoline (reagent preparation 3) in step 3. ¹H NMR (400 MHz, CD₃OD) δ 8.86 (d, 1H), 8.54 (s, 1H), 8.41 (d, 1H), 7.81 (d, 1H), 7.62 (dd, 1H), 7.12 (d, 1H), 5.19 (q, 2H), 4.70-4.48 (m, 2H), 4.27 (dd, 2H), 2.91 (s, 3H), 2.97-2.76 (m, 2H), 2.72-2.53 (m, 2H), 2.01 (dd, 1H), 1.62-1.46 (m, 1H), 1.46-1.32 (m, 3H), 0.83 (t, 3H); MS (ES) for C₂₆H₂₈N₆O: 441.2 (MH⁺).

7-(2-methyl-1H-imidazo[4,5-b]pyridin-6-yl)-4-(7-methyl-5,6,7,8-tetrahydroquinazolin-4-yl)-2,3,4,5-tetrahydro-1,4-benzoxazepine. Prepared as in example 6 using 4-chloro-7-methyl-5,6,7,8-tetrahydroquinazoline (reagent preparation 3) in step 3. ¹H NMR (400 MHz, CD₃OD) δ 8.48 (d, 1H), 8.31 (s, 1H), 8.02 (d, 1H), 7.57 (d, 1H), 7.48 (dd, 1H), 7.07 (d, 1H), 4.86-4.72 (m, 2H), 4.48-4.22 (m, 2H), 4.10-3.91 (m, 2H), 2.95-2.81 (m, 2H), 2.64 (s, 3H), 2.68-2.55 (m, 1H), 2.30 (dd, 1H), 2.05-1.86 (m, 2H), 1.29-1.13 (m, 1H), 1.09 (d, 3H); MS (ES) for C₂₅H₂₆N₆O: 427.2 (MH⁺).

4-(6,6-difluoro-5,6,7,8-tetrahydroquinazolin-4-yl)-7-(2-methyl. 1H-imidazo[4,5-b]pyridin-6-yl)-2,3,4,5-tetrahydro-1,4-benzoxazepine. The dihydrochloride salt was prepared as in example 6 using 4-chloro-6,6-difluoro-5,6,7,8-tetrahydroquinazoline (reagent preparation 3) in step 3. ¹H NMR (400 MHz, CD₃OD) δ 8.86 (d, 1H), 8.60 (s, 1H), 8.40 (d, 1H), 7.81 (d, 1H), 7.62 (dd, 1H), 7.11 (d, 1H), 5.20 (s, 2H), 4.54 (t, 2H), 4.34 (t, 2H), 3.46 (t, 2H), 3.10 (t, 2H), 2.90 (s, 3H), 2.50-2.34 (m, 2H). MS (ES) for C₂₄H₂₂F₂N₆O: 449.3 (MH⁺).

7-(2-methyl-1H-imidazo[4,5-b]pyridin-6-yl)-4-[(7R)-7-methyl-5,6,7,8-tetrahydroquinazolin-4-yl]-2,3,4,5-tetrahydro-1,4-benzoxazepine. The dihydrochloride salt was prepared as in example 6 using (R)-4-chloro-7-methyl-5,6,7,8-tetrahydroquinazoline (reagent preparation 3) in step 3. ¹H NMR (400 MHz, CD₃OD) δ 8.85 (d, 1H), 8.52 (s, 1H), 8.40 (d, 1H), 7.80 (d, 1H), 7.61 (dd, 1H), 7.11 (d, 1H), 5.26 (d, 1H), 5.17 (d, 1H), 4.64-4.42 (m, 2H), 4.42-4.17 (m, 2H), 3.10-2.83 (m, 2H), 2.90 (s, 3H), 2.74 (d, 1H), 2.40 (dd, 1H), 2.14-1.87 (m, 2H), 1.35-1.21 (m, 1H), 1.12 (d, 3H); MS (ES) for C₂₅H₂₆N₆O: 427.2 (MH⁺).

4-(2,6-dimethyl-5,6,7,8-tetrahydroquinazolin-4-yl)-7-(2-methyl-1H-imidazo[4,5-b]pyridin-6-yl)-2,3,4,5-tetrahydro-1,4-benzoxazepine. The dihydrochloride salt was prepare as in example 6 using 4-chloro-2,6-dimethyl-5,6,7,8-tetrahydroquinazoline (reagent preparation 3) in step 3. ¹H NMR (400 MHz, CD₃OD) δ 8.84 (s, 1H), 8.37 (s, 1H), 7.82 (s, 1H), 7.61 (d, 1H), 7.11 (d, 1H), 5.21 (d, 1H), 5.10 (d, 1H), 4.66-4.42 (m, 2H), 4.40-3.99 (m, 2H), 2.88 (s, 3H), 2.95-2.72 (m, 2H), 2.72-2.37 (m, 2H), 2.50 (s, 3H), 1.98-1.88 (m, 1H), 1.74-1.60 (m, 1H), 1.58-1.43 (m, 1H), 1.07 (d, 3H); MS (ES) for C₂₆H₂₈N₆O: 441.2 (MH⁺).

4-(6-ethyl-2-methyl-5,6,7,8-tetrahydroquinazolin-4-yl)-7-(2-methyl-1H-imidazo[4,5-b]pyridin-6-yl)-2,3,4,5-tetrahydro-1,4-benzoxazepine. The dihydrochloride salt was prepared as in example 6 using 4-chloro-6-ethyl-2-methyl-5,6,7,8-tetrahydroquinazoline (reagent preparation 3) in step 3. ¹H NMR (400 MHz, CD₃OD) δ 8.82 (d, 1H), 8.35 (d, 1H), 7.80 (d, 1H), 7.61 (dd, 1H), 7.12 (d, 1H), 5.19 (d, 1H), 5.11 (d, 1H), 4.68-4.46 (m, 2H), 4.44-4.01 (m, 2H), 2.87 (s, 3H), 2.96-2.71 (m, 2H), 2.69-2.42 (m, 2H), 2.51 (s, 3H), 2.01 (d, 1H), 1.59-1.30 (m, 4H), 0.82 (s, 3H); MS (ES) for C₂₇H₃₀N₆O: 455.3 (MH⁺).

7-(2-methyl-1H-imidazo[4,5-b]pyridin-6-yl)-4-[7-(trifluoromethyl)-5,6,7,8-tetrahydroquinazolin-4-yl]-2,3,4,5-tetrahydro-1,4-benzoxazepine. The dihydrochloride was prepared as in example 6 using 4-chloro-7-(trifluoromethyl)-5,6,7,8-tetrahydroquinazoline (reagent preparation 3) in step 3. ¹H NMR (400 MHz, CD₃OD) δ 8.86 (d, 1H), 8.57 (s, 1H), 8.42 (d, 1H), 7.82 (d, 1H), 7.61 (dd, 1H), 7.11 (d, 1H), 5.28 (d, 1H), 5.19 (d, 1H), 4.68-4.44 (m, 2H), 4.44-4.15 (m, 2H), 3.22-3.01 (m, 2H), 2.89 (s, 3H), 2.98-2.73 (m, 3H), 2.25 (d, 1H), 1.66-1.47 (m, 1H); MS (ES) for C₂₅H₂₃F₃N₆O: 481.2 (MH⁺).

4-[trans-6,7-dimethyl-5,6,7,8-tetrahydroquinazolin-4-yl]-7-(2-methyl-1H-imidazo[4,5-b]pyridin-6-yl)-2,3,4,5-tetrahydro-1,4-benzoxazepine. The dihydrochloride salt was prepared as in example 6 using trans-4-chloro-6,7-dimethyl-5,6,7,8-tetrahydroquinazoline (reagent preparation 3) in step 3. ¹H NMR (400 MHz, CD₃OD) δ 8.86 (s, 1H), 8.53 (s, 1H), 8.43 (s, 1H), 7.82 (s, 1H), 7.62 (d, 1H), 7.11 (d, 1H), 5.24 (s, 2H), 4.49 (s, 2H), 4.45-4.24 (m, 2H), 3.03-2.78 (m, 2H), 2.91 (s, 3H), 2.70-2.44 (m, 2H), 2.20-1.90 (m, 2H), 1.02 (d, 3H), 0.89 (d, 3H); MS (ES) for C₂₆H₂₈N₆O: 441.2 (MH⁺).

7-(2-methyl-1H-imidazo[4,5-b]pyridin-6-yl)-4-[(7S)-7-methyl-5,6,7,8-tetrahydroquinazolin-4-yl]-2,3,4,5-tetrahydro-1,4-benzoxazepine. The dihydrochloride salt was prepared as in example 6 using (S)-4-chloro-7-methyl-5,6,7,8-tetrahydroquinazoline, (reagent preparation 3) in step 3. ¹H NMR (400 MHz, CD₃OD) δ 8.84 (s, 1H), 8.52 (d, 1H), 8.39 (s, 1H), 7.79 (s, 1H), 7.60 (d, 1H), 7.11 (d, 1H), 5.27 (d, 1H), 5.18 (d, 1H), 4.65-4.43 (m, 2H), 4.32 (dd, 2H), 3.11-2.83 (m, 2H), 2.90 (s, 3H), 2.75 (d, 1H), 2.41 (dd, 1H), 2.10-1.89 (m, 2H), 1.36-1.22 (m, 1H), 1.12 (d, 3H); MS (ES) for C₂₅H₂₆N₆O: 427.2 (MH⁺).

7-(2-methyl-1H-imidazo[4,5-b]pyridin-6-yl)-4-[5-(trifluoromethyl)-5,6,7,8-tetrahydroquinazolin-4-yl]-2,3,4,5-tetrahydro-1,4-benzoxazepine. The dihydrochloride salt was prepared as in example 6 using 4-chloro-5-(trifluoromethyl)-5,6,7,8-tetrahydroquinazoline (reagent preparation 3) in step 3. ¹H NMR (400 MHz, CD₃OD) δ 8.86 (d, 1H), 8.51 (s, 1H), 8.40 (d, 1H), 7.81 (d, 1H), 7.60 (dd, 1H), 7.05 (d, 1H), 5.39 (d, 1H), 5.28 (d, 1H), 4.86-4.74 (m, 1H), 4.55-4.39 (m, 1H), 4.38-4.03 (m, 3H), 3.06-2.64 (m, 2H), 2.89 (s, 3H), 2.41-2.13 (m, 2H), 2.13-1.81 (m, 2H); MS (ES) for C₂₅H₂₃F₃N₆O: 481.2 (MH⁺).

7-(2-cyclopropyl-1H-imidazo[4,5-b]pyridin-6-yl)-4-[(7S)-7-methyl-5,6,7,8-tetrahydroquinazolin-4-yl]-2,3,4,5-tetrahydro 1,4-benzoxazepine. The dihydrochloride salt was prepared as in example 6 using isobutyl 6-bromo-2-cyclopropyl-1H-imidazo[4,5-b]pyridine-1-carboxylate (reagent preparation 19) in step 1 and (S)-4-chloro-7-methyl-5,6,7,8-tetrahydroquinazoline (reagent preparation 3) in step 3. ¹H NMR (400 MHz, CD₃OD) δ 8.78 (d, 1H), 8.52 (s, 1H), 8.39 (d, 1H), 7.80 (d, 1H), 7.60 (dd, 1H), 7.11 (d, 1H), 5.25 (d, 1H), 5.17 (d, 1H), 4.64-4.43 (m, 2H), 4.42-4.18 (m, 2H), 3.15-2.85 (m, 2H), 2.74 (d, 1H), 2.54-2.33 (m, 2H), 2.10-1.90 (m, 2H), 1.61-1.40 (m, 4H), 1.37-1.20 (m, 1H), 1.12 (d, 3H); MS (ES) for C₂₇H₂₈N₆O: 453.4 (MH⁺).

7-(2-methyl-1H-imidazo[4,5-b]pyridin-6-yl)-4-(7-methyl-7H-pyrrolo[2,3-d]pyrimidin-4-yl)-2,3,4,5-tetrahydro-1,4-benzoxazepine. The dihydrochloride salt was prepared as in example 6 using 4-chloro-7-methyl-7H-pyrrolo[2,3-d]pyrimidine in step 3. ¹H NMR (400 MHz, CD₃OD) δ 8.84 (s, 1H), 8.47 (s, 1H), 8.40 (s, 1H), 8.04-7.77 (m, 1H), 7.65-7.23 (m, 2H), 7.20-6.97 (m, 2H), 5.43 (s, 2H), 4.66-4.37 (m, 4H), 4.00 (s, 3H), 2.91 (s, 3H); MS (ES) for C₂₃H₂₁N₇O: 412.3 (MH⁺).

{4-[7-(2-methyl-1H-imidazo[4,5-b]pyridin-6-yl)-2,3-dihydro-1,4-benzoxazepin-4(5H)-yl]-5,6,7,8-tetrahydroquinazolin-7-yl}methanol. Prepared as in example 6 using 4-chloro-7-methyl-7H-pyrrolo[2,3-d]pyrimidine (reagent preparation 42) in step 3. ¹H NMR (400 MHz, d₆-DMSO) δ 8.50 (bs, 1H), 8.35 (s, 1H), 8.04 (bs, 1H), 7.69 (d, 1H), 7.54 (dd, 1H), 7.05 (d, 1H), 4.80-4.57 (m, 3H), 4.47-4.19 (m, 2H), 3.96-3.78 (m, 2H), 2.94-2.63 (m, 3H), 2.54 (s, 3H), 2.40-2.25 (m, 2H), 1.92 (bs, 2H), 1.20-1.00 (m, 1H); MS (ES) for C₂₅H₂₆N₆O₂: 443.4 (MH⁺).

1-{4-[7-(2-methyl-1H-imidazo[4,5-b]pyridin-6-yl)-2,3-dihydro-1,4-benzoxazepin-4(5H)-yl]-5,6,7,8-tetrahydroquinazolin-7-yl}ethanol. The dihydrochloride salt was prepared as in example 6 using 1-(4-chloro-5,6,7,8-tetrahydroquinazolin-7-yl)ethanol (reagent preparation 43) in step 3. ¹H NMR (400 MHz, CD₃OD) δ 8.62 (s, 1H), 8.42 (s, 1H), 8.17 (s, 1H), 7.68 (s, 1H), 7.48 (dd, 1H), 7.00 (d, 1H), 5.15 (d, 1H), 5.06 (d, 1H), 4.57-4.32 (m, 2H), 4.30-4.05 (m, 2H), 3.67-3.53 (m, 1H), 2.99-2.42 (m, 4H), 2.70 (s, 3H), 2.18-1.62 (m, 2H), 1.38-1.03 (m, 4H); MS (ES) for C₂₆H₂₈N₆O₂: 457.4 (MH⁺).

7-(2-methyl-1H-imidazo[4,5-b]pyridin-6-yl)-4-{7-[(methyloxy)methyl]-5,6,7,8-tetrahydroquinazolin-4-yl}-2,3,4,5-tetrahydro-1,4-benzoxazepine. The dihydrochloride salt was prepared as in example 6 using 4-chloro-7-(methoxymethyl)-5,6,7,8-tetrahydroquinazoline (reagent preparation 44) in step 3. ¹H NMR (400 MHz, CD₃OD) δ 8.74 (s, 1H), 8.53 (s, 1H), 8.31 (s, 1H), 7.77 (s, 1H), 7.58 (d, 1H), 7.10 (d, 1H), 5.24 (d, 1H), 5.16 (d, 1H), 4.64-4.41 (m, 2H), 4.41-4.17 (m, 2H), 3.46-3.33 (m, 2H), 3.35 (s, 3H), 2.82 (s, 3H), 3.09-2.49 (m, 4H), 2.29-2.11 (m, 1H), 2.07-1.96 (m, 1H), 1.42-1.28 (m, 1H); MS (ES) for C₂₆H₂₈N₆O₂: 457.4 (MH⁺).

4-(8,8-dimethyl-5,6,7,8-tetrahydroquinazolin-4-yl)-7-(2-methyl-1H-imidazo[4,5-b]pyridin-6-yl)-2,3,4,5-tetrahydro-1,4-benzoxazepine. The dihydrochloride salt was prepared as in example 6 using 4-chloro-8,8-dimethyl-5,6,7,8-tetrahydroquinazoline (reagent preparation 3) in step 3. ¹H NMR (400 MHz, CD₃OD) δ 8.74 (d, 1H), 8.52 (s, 1H), 8.30 (d, 1H), 7.77 (d, 1H), 7.58 (dd, 1H), 7.09 (d, 1H), 5.20 (s, 2H), 4.53-4.41 (m, 2H), 4.38-4.24 (m, 2H), 2.83 (s, 3H), 2.87-2.74 (m, 2H), 1.88-1.63 (m, 4H), 1.38 (s, 6H); MS (ES) for C₂₆H₂₈N₆O: 441.2 (MH⁺).

7-(2-methyl-1H-imidazo[4,5-b]pyridin-6-yl)-4-(6,6,7-trimethyl-5,6-dihydroquinazolin-4-yl)-2,3,4,5-tetrahydro-1,4-benzoxazepine. The dihydrochloride salt was prepared as in example 6 using 4-chloro-6,6,7-trimethyl-5,6-dihydroquinazoline (reagent preparation 3) in step 3. ¹H NMR (400 MHz, d₆-DMSO) δ 8.80 (s, 1H), 8.66 (s, 1H), 8.36 (s, 1H), 7.86 (s, 1H), 7.66 (d, 1H), 7.06 (d, 1H), 6.24 (s, 1H), 5.10 (s, 2H), 4.50 (bs, 2H), 4.17 (bs, 2H), 2.84 (s, 2H), 2.75 (s, 3H), 1.97 (s, 3H), 0.96 (s, 6H); MS (ES) for C₂₇H₂₈N₆O: 453.2 (MH⁺).

4-[(8S)-8-ethenyl-6,7,8,9-tetrahydro-5H-cyclohepta[d]pyrimidin-4-yl]-7-(2-methyl-1H-imidazo[4,5-b]pyridin-6-yl)-2,3,4,5-tetrahydro-1,4-benzoxazepine. The dihydrochloride salt was prepared as in example 6 using (S)-4-chloro-8-vinyl-6,7,8,9-tetrahydro-5H-cyclohepta[d]pyrimidine (reagent preparation 3) in step 3. ¹H NMR (400 MHz, CD₃OD) δ 8.75 (d, 1H), 8.47 (s, 1H), 8.29 (d, 1H), 7.73 (d, 1H), 7.58 (dd, 1H), 7.08 (d, 1H), 5.87 (ddd, 1H), 5.23-5.00 (m, 4H), 4.51 (t, 2H), 4.28-3.97 (m, 2H), 3.13-2.71 (m, 4H), 2.82 (s, 3H), 2.53-2.38 (m, 1H), 2.16-1.93 (m, 2H), 1.84-1.48 (m, 2H); MS (ES) for C₂₇H₂₈N₆O: 453.2 (MH⁺).

4-[(8S)-8-ethyl-6,7,8,9-tetrahydro-5H-cyclohepta[d]pyrimidin-4-yl]-7-(2-methyl-1H-imidazo[4,5-b]pyridin-6-yl)-2,3,4,5-tetrahydro-1,4-benzoxazepine. The dihydrochloride salt was prepared as in example 6 using (S)-4-chloro-8-vinyl-6,7,8,9-tetrahydro-5H-cyclohepta[d]pyrimidine (reagent preparation 3) in step 3, followed by hydrogenation. ¹H NMR (400 MHz, CD₃OD) δ 8.77 (d, 1H), 8.47 (s, 1H), 8.32 (d, 1H), 7.74 (d, 1H), 7.59 (dd, 1H), 7.08 (d, 1H), 5.18-5.04 (m, 2H), 4.51 (t, 2H), 4.25-4.05 (m, 2H), 2.97-2.67 (m, 2H), 2.84 (s, 3H), 2.12-1.86 (m, 2H), 1.69-1.52 (m, 3H), 1.47-1.33 (m, 2H), 0.98 (t, 3H); MS (ES) for C₂₇H₃₀N₆O: 455.2 (MH⁺).

1-{(7S)-7-ethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}-N,N-dimethylmethanamine. Prepared as in example 6 using (S)-1-(4-chloro-7-ethyl-5,6,7,8-tetrahydroquinazolin-2-yl)-N,N-dimethylmethanamine, (reagent preparation 17) in step 3. ¹H NMR (400 MHz, d₆-DMSO) δ 12.33 (m, 1H), 8.48 (d, 1H), 7.96 (d, 1H), 7.63 (d, 1H), 7.47 (dd, 1H), 7.01 (d, 1H), 4.71 (s, 2H), 4.34 (dd, 2H), 4.04-3.78 (m, 2H), 3.34 (s, 2H), 2.81 (dd, 2H), 2.64-2.50 (m, 4H), 2.27 (dd, 1H), 2.17 (d, 6H), 1.88 (d, 1H), 1.70 (s, 1H), 1.45-1.06 (m, 3H), 0.94 (t, 3H); MS (ES) for C₂₉H₃₅N₇O: 498.2 (MH⁺).

4-(2-{[(3R)-3-fluoropyrrolidin-1-yl]methyl}-6,6-dimethyl-5,6,7,8-tetrahydroquinazolin-4-yl)-7-(2-methyl-1H-imidazo[4,5-b]pyridin-6-yl)-2,3,4,5-tetrahydro-1,4-benzoxazepine. The trihydrochloride salt was prepared as in example 6 using (R)-4-chloro-2-((3-fluoropyrrolidin-1-yl)methyl)-6,6-dimethyl-5,6,7,8-tetrahydroquinazoline (reagent preparation 17) in step 3. ¹H NMR (400 MHz, CD₃OD) δ 8.94 (s, 1H), 8.52 (s, 1H), 8.00 (s, 1H), 7.69 (d, 1H), 7.13 (d, 1H), 5.40-5.21 (m, 3H), 4.77-4.64 (m, 2H), 4.64-4.55 (m, 2H), 4.42-4.27 (m, 2H), 3.63 (s, 4H), 2.90 (s, 3H), 2.94-2.81 (m, 2H), 2.61 (s, 2H), 2.23 (s, 2H), 1.72 (t, 2H), 0.93 (d, 6H); MS (ES) for C₃₁H₃₆FN₇O: 542.4 (MH⁺).

4-(2-{[(3R)-3-fluoropyrrolidin-1-yl]methyl}-6,6-dimethyl-5,6,7,8-tetrahydroquinazolin-4-yl)-7-(2-methyl-1H-benzimidazol-6-yl)-2,3,4,5-tetrahydro-1,4-benzoxazepine. The trihydrochloride salt was prepared as in example 6 using (S)-4-chloro-2-((3-fluoropyrrolidin-1-yl)methyl)-6,6-dimethyl-5,6,7,8-tetrahydroquinazoline (reagent preparation 17) in step 3. ¹H NMR (400 MHz, d₆-DMSO) δ 8.95 (d, 1H), 8.53 (d, 1H), 8.17 (s, 1H), 7.71 (dd, 1H), 7.07 (d, 1H), 5.37-5.03 (m, 3H), 4.63-4.52 (m, 2H), 4.52-4.46 (m, 2H), 4.16 (bs, 2H), 3.73-3.22 (m, 4H), 2.82 (s, 3H), 2.79 (t, 2H), 2.55 (s, 2H), 2.25-2.01 (m, 2H), 1.60 (t, 2H), 0.87 (d, 6H); MS (ES) for C₃₁H₃₆FN₇O: 542.4 (MH⁺).

4-(6,6-dimethyl-2-pyridin-2-yl-5,6,7,8-tetrahydroquinazolin-4-yl)-7-(2-methyl-1H-imidazo[4,5-b]pyridin-6-yl)-2,3,4,5-tetrahydro-1,4-benzoxazepine. The trihydrochloride salt was prepared as in example 6 using 4-chloro-6,6-dimethyl-2-(pyridin-2-yl)-5,6,7,8-tetrahydroquinazoline (reagent preparation 17) in step 3. ¹H NMR (400 MHz, d₆-DMSO) δ 8.90 (s, 1H), 8.81 (d, 1H), 8.54 (d, 1H), 8.48 (s, 1H), 8.25 (s, 1H), 8.07 (t, 1H), 7.71 (dd, 1H), 7.66 (dd, 1H), 7.01 (d, 1H), 5.31 (s, 2H), 4.59 (bs, 2H), 4.33 (bs, 2H), 2.95 (t, 2H), 2.87 (s, 3H), 2.66 (s, 2H), 1.62 (t, 2H), 0.93 (s, 6H); MS (ES) for C₃₁H₃₁N₇O: 518.3 (MH⁺).7-(2-methyl-1H-imidazo[4,5-b]pyridin-6-yl)-4-(5-methylpyrimidin-4-yl)-2,3,4,5-tetrahydro-1,4-benzoxazepine. Synthesized according to the method of example 6 using 4-chloro-5-methylpyrimidine in step 3. ¹H NMR (400 MHz, d₆-DMSO): 8.89 (d, 1H), 8.80 (s, 1H), 8.45 (d, 1H), 8.28 (s, 1H), 7.91 (d, 1H), 7.69 (dd, 1H), 7.10 (d, 1H), 5.27 (s, 2H), 4.50 (m, 2H), 4.33 (m, 2H), 2.84 (s, 3H), 2.43 (s, 3H), 1.76 (s, 3H); MS (EI) for C₂₁H₂₀N₆O: 373 (MH⁺).

4-[2,6-dimethyl-5-(1-methylethyl)pyrimidin-4-yl]-7-(2-methyl-1H-imidazo[4,5-b]pyridin-6-yl)-2,3,4,5-tetrahydro-1,4-benzoxazepine. Synthesized according to the method of example 6 using 4-chloro-5-isopropyl-2,6-dimethylpyrimidine (reagent preparation 8) in step 3. ¹H NMR (400 MHz, d₆-DMSO): 8.74 (s, 1H), 8.29 (s, 1H), 7.82 (s, 1H), 7.63 (dd, 1H), 7.30 (s, 1H), 7.17 (s, 1H), 7.04 (s, 1H), 5.06 (s, 2H), 4.50 (m, 2H), 4.03 (m, 2H), 3.08 (m, 1H) 2.71 (s, 3H), 2.54 (s, 3H), 2.41 (s, 3H), 1.32 (d, 6H); MS (EI) for C₂₅H₂₈N₆O: 429 (MH⁺).

7-(2-methyl-1H-imidazo[4,5-b]pyridin-6-yl)-4-[6-methyl-5-(1-methylethyl)pyrimidin-4-yl]-2,3,4,5-tetrahydro-1,4-benzoxazepine. Synthesized according to the method of example 6 using 4-chloro-5-isopropyl-6-methylpyrimidine (reagent preparation 5) in step 3. ¹H NMR (400 MHz, d₆-DMSO): 8.81 (s, 1H), 8.64 (s, 1H), 8.36 (s, 1H), 7.65 (d, 1H), 7.03 (d, 1H), 5.04 (s, 2H), 4.53 (s, 2H), 4.02 (s, 2H), 3.11 (m, 1H), 2.76 (s, 3H), 2.56 (s, 3H), 1.34 (d, 6H); MS (EI) for C₂₄H₂₆N₆O: 415 (MH⁺).

7-(2-methyl-1H-imidazo[4,5-b]pyridin-6-yl)-4-(7-methyl-7-phenyl-5,6,7,8-tetrahydroquinazolin-4-yl)-2,3,4,5-tetrahydro-1,4-benzoxazepine. Synthesized according to the method of example 6 using 4-chloro-7-methyl-7-phenyl-5,6,7,8-tetrahydroquinazoline (reagent preparation 3) in step 3. ¹H NMR (400 MHz, d₆-DMSO): 8.49 (s, 1H), 8.37 (s, 1H), 7.92 (d, 1H), 7.48 (d, 1H), 7.31 (s, 1H), 7.29 (s, 1H), 7.22 (d, 1H), 7.13 (m, 2H), 7.02 (m, 1H), 6.94-6.80 (m, 1H), 4.42-4.56 (m, 2H), 4.32 (m, 2H), 3.94 (d, 1H), 3.76 (m, 1H), 3.14 (d, 1H), 2.81 (d, 1H), 2.68 (d, 1H), 2.55 (d, 3H), 2.55 (d, 3H), 2.29 (m, 1H), 2.09 (m, 1H), 1.84 (m, 1H), 1.32 (s, 3H); MS (EI) for C₃₁H₃₀N₆O: 503 (MH⁺).

N,N,2-trimethyl-4-[7-(2-methyl-1H-imidazo[4,5-b]pyridin-6-yl)-2,3-dihydro-1,4-benzoxazepin-4(5H)-yl]pyrimidine-5-sulfonamide. Synthesized according to the method of example 6 using 4-chloro-N,N,2-trimethylpyrimidine-5-sulfonamide in step 3. ¹H NMR (400 MHz, d₆-DMSO): 8.51 (s, 1H), 8.03 (s, 1H), 7.94 (s, 1H), 7.52 (dd, 1H) 7.04 (d, 1H), 6.71 (d, 1H), 5.05 (s, 2H), 4.31 (d, 2H), 4.24 (d, 2H), 2.84 (s, 6H), 2.54 (s, 3H), 2.40 (s, 3H); MS (EI) for C₂₃H₂₅N₇O₃S: 480 (MH⁺).

7-(2-methyl-1H-imidazo[4,5-b]pyridin-6-yl)-4-[2-methyl-5-(morpholin-4-ylsulfonyl)pyrimidin-4-yl]-2,3,4,5-tetrahydro-1,4-benzoxazepine. Synthesized according to the method of example 6 using 4-(4-chloro-2-methylpyrimidin-5-ylsulfonyl)morpholine in step 3. ¹H NMR (400 MHz, d₆-DMSO): 8.58 (s, 1H), 8.50 (d, 1H), 8.03 (d, 1H), 7.81 (s, 1H), 7.53 (d, 1H), 7.03 (d, 1H), 5.04 (s, 2H), 4.29 (s, 2H), 3.61 (s, 2H), 3.12 (s, 2H), 2.42 (s, 3H); MS (EI) for C₂₅H₂₇N₇O₄S: 522 (MH⁺).

7-(2-cyclopropyl-1H-imidazo[4,5-b]pyridin-6-yl)-4-(6,6-dimethyl-5,6-dihydroquinazolin-4-yl)-2,3,4,5-tetrahydro-1,4-benzoxazepine. Synthesized according to the method of example 6 using isobutyl 6-bromo-2-cyclopropyl-1H-imidazo[4,5-b]pyridine-1-carboxylate (reagent preparation 19) in step 1 and 4-chloro-6,6-dimethyl-5,6-dihydroquinazoline (reagent preparation 3) in step 3. ¹H NMR (400 MHz, d₆-DMSO): 8.64 (s, 1H), 8.61 (s, 1H), 8.14 (m, 1H), 7.77 (d 1H), 7.59 (dd, 1H), 7.04 (d, 1H), 6.47 (d, 1H), 6.34 (d, 1H), 5.00 (s, 2H), 4.46 (m, 2H), 4.11 (s, 2H), 2.83 (s, 2H), 2.24 (m, 1H), 1.24-1.18 (m, 5H), 0.99 (s, 6H); MS (EI) for C₂₈H₂₈N₆O: 465 (MH⁺).

4-(6,6-dimethyl-5,6-dihydroquinazolin-4-yl)-7-(2-methyl-1H-imidazo[4,5-b]pyridin-6-yl)-2,3,4,5-tetrahydro-1,4-benzoxazepine. Synthesized according to the method of example 6 using 4-chloro-6,6-dimethyl-5,6-dihydroquinazoline (reagent preparation 3) in step 3. ¹H NMR (400 MHz, d₆-DMSO): 8.64 (s, 1H), 8.59 (s, 1H), 8.18 (s, 1H), 7.77 (d, 1H), 7.60 (dd, 1H), 7.05 (d, 1H), 6.43 (d, 1H), 6.32 (d, 1H), 4.94 (s, 2H), 4.43 (m, 2H), 4.06 (br s, 2H), 2.82 (s, 2H), 2.62 (s, 3H), 0.99 (s, 6H); MS (EI) for C₂₆H₂₆N₆O: 439 (MH⁺).

7-(2-methyl-1H-imidazo[4,5-b]pyridin-6-yl)-4-(6,6,8-trimethyl-5,6-dihydroquinazolin-4-yl)-2,3,4,5-tetrahydro-1,4-benzoxazepine. Synthesized according to the method of example 6 using 4-chloro-6,6,8-trimethyl-5,6-dihydroquinazoline (reagent preparation 3) in step 3. ¹H NMR (400 MHz, d₆-DMSO): 8.74 (d, 1H), 8.54 (s, 1H), 8.30 (d, 1H), 7.77 (d, 1H), 7.61 (dd, 1H), 7.05 (d, 1H), 6.09 (s, 1H), 4.82 (s, 2H), 4.42 (m, 2H), 3.96 (br s, 2H), 2.73 (s, 2H), 2.69 (s, 3H), 1.96 (d, 3H), 0.92 (s, 6H); MS (EI) for C₂₇H₂₆N₆O: 453 (MH⁺).

7-(2-methyl-1H-imidazo[4,5-b]pyridin-6-yl)-4-(6,6,7,8-tetramethyl-5,6-dihydroquinazolin-4-yl)-2,3,4,5-tetrahydro-1,4-benzoxazepine. Synthesized according to the method of example 6 using 4-chloro-6,6,7,8-tetramethyl-5,6-dihydroquinazoline (reagent preparation 3) in step 3. ¹H NMR (400 MHz, d₆-DMSO): 8.69 (s, 1H), 8.53 (s, 1H), 8.24 (s, 1H), 7.77 (d, 1H), 7.61 (dd, 1H), 7.06 (d, 1H), 4.86 (s, 2H), 4.42 (m, 2H), 4.00 (br s, 2H), 2.71 (s, 2H), 2.66 (s, 3H), 1.97 (s, 3H), 1.87 (s, 3H), 0.88 (s, 6H); MS (EI) for C₂₈H₃₀N₆O: 467 (MH⁺).

N,N-dimethyl-1-{4-[7-(2-methyl-1H-imidazo[4,5-b]pyridin-6-yl)-2,3-dihydro-1,4-benzoxazepin-4(5H)-yl]-7-(methyloxy)quinazolin-2-yl}methanamine. Synthesized according to the method of example 6 using 1-(4-chloro-7-methoxyquinazolin-2-yl)-N,N-dimethylmethanamine (reagent preparation 20) in step 3. ¹H NMR (400 MHz, d₆-DMSO): 8.59-8.50 (br d, 1H), 8.11-7.98 (br d, 1H), 7.94 (d, 1H), 7.76 (d, 1H), 7.53 (dd, 1H), 7.16 (d, 1H), 7.08 (dd, 1H), 6.99 (d, 1H), 5.06 (s, 2H), 4.46 (s, 2H), 4.21 (m, 2H), 3.89 (s, 3H), 3.45 (s, 2H), 2.52 (s, 3H), 2.13 (s, 6H), 1.91 (s, 2H); MS (EI) for C₂₈H₂₉N₇O₂: 496 (MH⁺).

1-{6-fluoro-4-[7-(2-methyl-1H-imidazo[4,5-b]pyridin-6-yl)-2,3-dihydro-1,4-benzoxazepin-4(5H)-yl]quinazolin-2-yl}-N,N-dimethylmethanamine. Synthesized according to the method of example 6 using 1-(4-chloro-6-fluoroquinazolin-2-yl)-N,N-dimethylmethanamine (reagent preparation 20) in step 3. ¹H NMR (400 MHz, d₆-DMSO): 8.63-8.54 (br d, 1H), 8.15-8.03 (br d, 1H), 7.90-7.75 (m, 4H), 7.58 (dd, 1H), 7.02 (d, 1H), 5.16 (s, 1H), 4.54 (br m, 2H), 4.27 (br m, 2H), 4.00 (br s, 2H), 3.34 (s, 6H), 2.54 (s, 3H); MS (EI) for C₂₇H₂₆FN₇O: 484 (MH⁺).

4-{2-[(3,3-difluoropyrrolidin-1-yl)methyl]-6,6-dimethyl-5,6,7,8-tetrahydroquinazolin-4-yl}-7-(2-methyl-1H-imidazo[4,5-b]pyridin-6-yl)-2,3,4,5-tetrahydro-1,4-benzoxazepine. Synthesized according to the method of example 6 using 2-((3,3-difluoropyrrolidin-1-yl)methyl)-4,6,6-trimethyl-5,6,7,8-tetrahydroquinazoline (reagent preparation 17) in step 3. ¹H NMR (400 MHz, d₆-DMSO): 8.88 (d, 1H), 8.44 (d, 1H), 7.99 (s, 1H), 7.67 (dd, 1H), 7.05 (d, 1H), 5.08 (s, 1H), 4.49 (br m, 2H), 4.15 (br m, 2H), 4.10 (br s, 2H), 3.24 (br t, 2H), 3.10 (br m, 2H), 2.81 (m, 4H), 2.54 (s, 2H), 2.31-2.23 (m, 2H), 1.59 (t, 2H), 0.87 (s, 6H); MS (EI) for C₃₁H₃₅F₂N₇O: 560 (MH⁺).

N-({6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl)-N-methylethanamine. Prepared according to the method of example 6 by using N-((4-chloro-6,6-dimethyl-5,6,7,8-tetrahydroquinazolin-2-yl)methyl)-N-methylethanamine (reagent preparation 17) in step 3. ¹H NMR (400 MHz, DMSO-d₆): 12.70 (d, 1H), 8.53 (d, 1H), 8.02 (d, 1H), 7.71 (d, 1H), 7.55-7.50 (m, 1H), 7.03 (d, 1H), 4.64 (s, 2H), 4.31 (m, 2H), 3.86 (m, 2H), 3.44 (s, 2H), 2.68 (t, 2H), 2.54 (s, 3H), 2.45 (s, 2H), 2.38 (q, 2H), 2.13 (s, 3H), 1.59 (t, 2H), 0.91 (t, 3H), 0.85 (s, 6H); MS (EI) for C₃₀H₃₇N₇O: 512 (MH⁺).

4-[6,6-dimethyl-2-(piperidin-1-ylmethyl)-5,6,7,8-tetrahydroquinazolin-4-yl]-7-(2-methyl-1H-imidazo[4,5-b]pyridin-6-yl)-2,3,4,5-tetrahydro-1,4-benzoxazepine. Prepared according to the method of example 6 by using 4-chloro-6,6-dimethyl-2-(piperidin-1-ylmethyl)-5,6,7,8-tetrahydroquinazoline (reagent preparation 17) in step 3. ¹H NMR (400 MHz, (DMSO-d₆): 12.70 (d, 1H), 8.52 (m, 1H), 8.00 (m, 1H), 7.70 (d, 1H), 7.54-7.50 (m, 1H), 7.02 (m, 1H), 4.66 (s, 2H), 4.33 (m, 2H), 3.87 (m, 2H), 2.68 (t, 2H), 2.56 (s, 2H), 2.46 (s, 2H), 2.33 (m, 4H), 1.59 (t, 2H), 1.38 (m, 4H), 1.26 (m, 2H). 0.86 (s, 6H); MS (EI) for C₃₂H₃₉N₇O: 538 (MH⁺).

N-({6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl)-N-methylpropan-2-amine. Prepared according to the method of example 6 by using N-((4-chloro-6,6-dimethyl-5,6,7,8-tetrahydroquinazolin-2-yl)methyl)-N-methylpropan-2-amine (reagent preparation 17) in step 3. ¹H NMR (400 MHz, DMSO-d₆): 12.68 (d, 1H), 8.52 (m, 1H), 8.01 (m, 1H), 7.71 (d, 1H), 7.55-7.50 (m, 1H), 7.03 (m, 1H), 4.65 (s, 2H), 4.31 (m, 2H), 3.86 (m, 2H), 2.69 (t, 2H), 2.56 (s, 2H), 2.45 (s, 2H), 2.11 (m, 3H), 1.59 (t, 2H), 1.23 (m, 2H), 0.91-0.84 (m, 12H); MS (EI) for C₃₁H₃₉N₇O: 526 (MH⁺).

N-({6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl)-N-methylcyclopropanamine. Prepared according to the method of example 6 by using N-((4-chloro-6,6-dimethyl-5,6,7,8-tetrahydroquinazolin-2-yl)methyl)-N-methylcyclopropanamine (reagent preparation 17) in step 3. ¹H NMR (400 MHz, Methanol-d₄): 8.48 (s, 1H), 8.01 (s, 1H), 7.58 (d, 1H), 7.46-7.41 (m, 1H), 7.02 (d, 1H), 4.73 (s, 2H), 4.31 (t, 2H), 3.96 (t, 2H), 3.62 (s, 2H), 2.75 (t, 2H), 2.61 (s, 3H), 2.46 (s, 2H), 2.30 (s, 3H), 1.83-1.76 (m, 1H), 1.64 (t, 2H), 0.89 (s, 6H), 0.32-0.23 (m, 4H); MS (EI) for C₃₁H₃₇N₇O: 524 (MH⁺).

N-({6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-yl}methyl)propan-2-amine. Prepared according to the method of example 6 by using benzyl (4-chloro-6,6-dimethyl-5,6,7,8-tetrahydroquinazolin-2-yl)methyl(isopropyl)carbamate (reagent preparation 17) in step 3 and Cbz group removal. ¹H NMR (400 MHz, DMSO-d₆): 12.7 (d, 1H), 8.60-8.46 (m, 1H), 8.11-7.94 (m, 1H), 7.72 (s, 1H), 7.54 (d, 1H), 7.03 (d, 1H), 4.68 (s, 2H), 4.32 (m, 2H), 3.89 (m, 2H), 3.65 (s, 2H), 2.69 (t, 2H), 2.53 (m, 1H), 2.46 (s, 2H), 1.59 (t, 2H), 1.23 (s, 1H), 0.92 (d, 6H), 0.86 (s, 6H); MS (EI) for C₃₀H₃₇N₇O: 512 (MH⁺).

1-{5-[(4-fluorophenyl)methyl]-4-methyl-6-[7-(2-methyl-3H-imidazo[4,5-b]pyridin-6-yl)-2,3-dihydro-1,4-benzoxazepin-4(5H)-yl]pyrimidin-2-yl}-N,N-dimethylmethanamine. Synthesized according to the method of example 6 using 1-(4-chloro-5-(4-fluorobenzyl)-6-methylpyrimidin-2-yl)-N,N-dimethylmethanamine (reagent preparation 17) in step 3. ¹H NMR (400 MHZ, CD₃OD): 8.38 (d, 1H), 7.92 (d, 1H), 7.44 (dd, 1H), 7.09 (dd, 2H), 7.03-6.95 (m, 3H), 6.92 (d, 1H), 4.60 (s, 2H), 4.28 (tr, 2H), 4.01 (s, 2H), 3.92 (tr, 2H), 3.54 (s, 2H), 2.66 (s, 3H), 2.30 (s, 6H), 2.21 (s, 3H). MS (EI) for C₃₁H₃₂N₇OF: 538 (MH⁺).

N,N-dimethyl-1-{4-methyl-5-(1-methylethyl)-6-[7-(2-methyl-3H-imidazo[4,5-b]pyridin-6-yl)-2,3-dihydro-1,4-benzoxazepin-4(5H)-yl]pyrimidin-2-yl}methanamine. Synthesized according to the method of example 6 using 1-(4-chloro-5-isopropyl-6-methylpyrimidin-2-yl)-N,N-dimethylmethanamine (reagent preparation 17) in step 3. ¹H NMR (400 MHZ, CDCl₃): 12.78 (br, 1H), 8.51 (s, 1H), 8.08 (br, 1H), 7.46 (d, 1H), 7.43 (s, 1H), 7.14 (d, 1H), 4.37 (s, 2H), 4.30 (tr, 2H), 3.76 (tr, 2H), 3.55 (s, 2H), 3.40 (m, 1H), 2.75 (s, 3H), 2.55 (s, 3H), 2.34 (s, 6H), 1.35 (d, 6H). MS (EI) for C₂₈H₃₄N₇O: 472 (MH⁺). 7-(2-methyl-3H-imidazo[4,5-b]pyridin-6-yl)-4-[2-methyl-7-(methyloxy)quinazolin-4-yl]-2,3,4,5-tetrahydro-1,4-benzoxazepine. Synthesized according to the method of example 6 using 4-chloro-7-methoxy-2-methylquinazoline in step 3. ¹H NMR (400 MHz, DMSO-d₆) δ 8.53 (s, 1H), 8.06 (s, 1H), 7.90 (d, 1H), 7.74 (d, 1H), 7.56 (dd, 1H), 7.11 (d, 1H), 7.03 (m, 3H), 5.02 (s, 3H), 4.41 (t, 3H), 4.11 (d, J=36.2 Hz, 3H), 3.88 (s, 5H), 2.55 (s, 5H), 2.43 (s, 5H), 1.90 (s, 1H); MS (EI) for C₂₆H₂₄N₆O₂: 453.3 (MH⁺).

{4-[7-(2-amino[1,3]thiazolo[5,4-b]pyridin-6-yl)-2,3-dihydro-1,4-benzoxazepin-4(5H)-yl]-6,6-dimethyl-5,6,7,8-tetrahydroquinazolin-2-yl}methanol. The dihydrochloride salt was prepared as in example 6 using (4-chloro-6,6-dimethyl-5,6,7,8-tetrahydroquinazolin-2-yl)methyl acetate (reagent preparation 17) in step 3 and acetate ester hydrolysis. ¹H NMR (400 MHz, CD₃OD) δ 8.66 (d, 1H), 8.02 (d, 1H), 7.79 (d, 1H), 7.57 (dd, 1H), 7.09 (d, 1H), 5.14 (s, 2H), 4.62 (s, 2H), 4.52-4.39 (m, 2H), 4.37-4.25 (m, 2H), 2.88 (t, 2H), 2.58 (s, 2H), 1.69 (t, 2H), 0.94 (s, 6H); MS (ES) for C₂₆H₂₈N₆O₂S: 498.2 (MH⁺).

7-(2-methyl-1H-imidazo[4,5-b]pyridin-6-yl)-4-[6-methyl-5-(1-methylpropyl)pyrimidin-4-yl]-2,3,4,5-tetrahydro-1,4-benzoxazepine. Prepared according to the method of example 6 by using 4-chloro-6-methylpyrimidine in step 3. ¹H NMR (400 MHz, methanol-d₄): 8.52 (s, 1H), 8.06 (s, 1H), 8.01 (d, 1H), 7.76 (d, 1H), 7.49 (dd, 1H), 7.10 (d, 1H), 6.74 (d, 1H), 4.24 (br. s, 2H), 4.19 (m, 2H), 2.64 (s, 3H), 2.42 (s, 3H); MS (EI) for C₂₁H₂₀N₆O: 373 (MH⁺).

### Example 7

### 7-(1-Methyl-1H-indazol-5-yl)-4-quinolin-4-yl-2,3,4,5-tetrahydro-1,4-benzoxazepine

STEP 1: A solution of 7-bromo-2,3,4,5-tetrahydro-1,4-benzoxazepine hydrochloride (1.50 g, 5.67 mmol) (example 2, step 1), 4-chloroquinoline (0.973 g, 5.95 mmol), diisopropylethylamine (2.93 g, 22.7 mmol) in 1-butanol (15 mL) was stirred at 170° C. for 1 hour in a microwave synthesizer. The reaction mixture was diluted with ethyl acetate (100 mL), washed with saturated sodium bicarbonate (50 mL) and brine (50 mL), dried over sodium sulfate. Filtration and concentration afforded a crude brown oil that was purified by silica gel chromatography (9:1 dichloromethane/methanol) to provide 7-bromo-4-quinolin-4-yl-2,3,4,5-tetrahydro-1,4-benzoxazepine (1.70 g, 84.6% yield) as a yellow oil. MS (EI) for C₁₈H₁₅BrN₂O: 356 (MH⁺).

STEP 2: A mixture of 7-bromo-4-quinolin-4-yl-2,3,4,5-tetrahydro-1,4-benzoxazepine (206 mg, 0.58 mmol), 5-(4,4,5,5-tetramethyl-1,3,2-dioxaborolan-2-yl)-1-methyl-1H-indazole (158 mg (0.58 mmol) (reagent preparation 50), potassium carbonate (320 mg, 2.32 mmol), and dichloro[1,1-bis(diphenylphosphino]ferrocenepalladium (II) dichloromethane adduct (50 mg, 0.06 mmol) in dimethoxyethane (4.0 mL) and water (1.0 mL) was degassed with nitrogen, and then stirred at 100° C. for 1 h. The reaction mixture was cooled to room temperature and partitioned between water and ethyl acetate. The mixture was filtered through celite and then the layers were separated. The organic layer was washed with brine, dried over anhydrous sodium sulfate, filtered and concentrated. Column chromatography on silica (25-100% ethyl acetate in hexanes) afforded the title Compound (23 mg, 10% yield) as a yellow solid. ¹H NMR (400 MHz, DMSO-d₆): 8.63 d, 1H), 8.11 (s, 1H), 8.03 (m, 2H), 7.95 (d, 1H), 7.75 (m, 3H), 7.70 (m, 1H), 7.61 (dd, 1H), 7.51 (m, 1H), 7.11 (d, 1H), 7.02 (d, 1H), 4.61 (s, 2H), 4.39 (m, 2H), 4.09 (s, 3H), 3.84 (m, 2H); MS (EI) for C₂₆H₂₂N₄O: 407 (MH⁺).

Using analogous synthetic techniques and substituting with alternative starting reagents in steps 1 or 2 the following compounds of the invention were prepared. Alternative starting materials were obtained commercially unless otherwise indicated.

7-(1H-indazol-5-yl)-4-quinolin-4-yl-2,3,4,5-tetrahydro-1,4-benzoxazepine. Prepared according to the method of example 7 by using 5-(4,4,5,5-tetramethyl-1,3,2-dioxaborolan-2-yl)-1H-imidazole in step 2. ¹H NMR (400 MHz, methanol-d₄): 8.41 (s, 1H), 8.38 (d, 1H), 8.34 (d, 1H), 8.14 (s, 1H), 7.97 (m, 1H), 7.90 (m, 2H), 7.83 (d, 1H), 7.73 (d, 1H), 7.69 (m, 1H), 7.62 (dd, 1H), 7.07 (m, 2H), 5.28 (s, 2H), 4.63 (m, 2H), 4.44 (m, 2H); MS (EI) for C₂₅H₂₀N₄O: 393 (MH⁺).

7-(1H-indazol-6-yl)-4-quinolin-4-yl-2,3,4,5-tetrahydro-1,4-benzoxazepine. Prepared as the trifluoroacetate salt according to the method of example 7 by using tert-butyl 6-(4,4,5,5-tetramethyl-1,3,2-dioxaborolan-2-yl)-1H-indazole-1-carboxylate in step 2. ¹H NMR (400 MHz, d₆-DMSO): 13.18 (br. s, 1H), 8.56 (d, 1H), 8.33 (d, 1H), 8.12 (s, 1H), 8.03-7.92 (m, 3H), 7.87 (d, 1H), 7.79 (s, 1H), 7.72-7.62 (m, 2H), 7.50 (d, 1H), 7.02-6.96 (m, 2H), 5.31 (s, 2H), 4.63 (t, 2H), 4.42 (t, 2H); MS (EI) for C₂₅H₂₀N₄O: 393 (MH⁺).

7-(1H-pyrazol-4-yl)-4-quinolin-4-yl-2,3,4,5-tetrahydro-1,4-benzoxazepine. Prepared as the trifluoroacetate salt according to the method of example 7 by using 4-(4,4,5,5-tetramethyl-1,3,2-dioxaborolan-2-yl)-1H-pyrazole in step 2. ¹H NMR (400 MHz, d₆-DMSO): 8.57 (d, 1H), 8.31 (d, 1H), 8.08-7.92 (m, 4H), 7.83 (s, 1H), 7.68 (t, 1H), 7.47 (d, 1H), 6.94 (d, 1H), 6.89 (d, 1H), 5.20 (s, 2H), 4.56 (t, 2H), 4.38 (t, 2H); MS (EI) for C₂₁H₁₈N₄O: 343 (MH⁺).

7-(2,3-dihydro-1-benzofuran-5-yl)-4-quinolin-4-yl-2,3,4,5-tetrahydro-1,4-benzoxazepine. Prepared as trifluoroacetate salt according to the method of example 7 by using 2,3-dihydro-1-benzofuran-1-ylboronic acid in step 2. ¹H NMR (400 MHz, methanol-d₄): 8.37 (d, 1H), 8.32 (d, 1H), 7.97 (m, 1H), 7.89 (d, 1H), 7.67 (m, 2H), 7.51 (s, 1H), 7.46 (dd, 1H), 7.37 (d, 1H), 7.05 (d, 1H), 6.98 (d, 1H), 6.80 (d, 1H), 5.22 (s, 2H), 4.59 (m, 4H), 4.41 (m, 2H), 3.26 (m, 2H); MS (EI) for C₂₆H₂₂N₂O₂: 395 (MH⁺).

7-(1-methyl-1H-indol-5-yl)-4-quinolin-4-yl-2,3,4,5-tetrahydro-1,4-benzoxazepine. Prepared according to the method of example 7 by using 1-methyl-1H-indol-5-ylboronic acid in step 2. ¹H NMR (400 MHz, DMSO-d₆): 8.57 (d, 1H), 8.34 (d, 1H), 7.96 (m, 4H), 7.88 (s, 1H), 7.69 (m, 1H), 7.55 (m, 3H), 7.38 (m, 1H), 6.98 (m, 2H), 6.50 (m, 1H), 5.29 (s, 2H), 4.60 (m, 2H), 4.42 (m, 2H), 3.84 (s, 3H); MS (EI) for C₂₇H₂₃N₃O: 406 (MH⁺).

N,N-dimethyl-3-(4-quinolin-4-yl-2,3,4,5-tetrahydro-1,4-benzoxazepin-7-yl)benzamide. Prepared as the trifluoroacetate salt according to the method of example 7 by 3-(dimethylcarbamoyl)phenylboronic acid in step 2. ¹H NMR (400 MHz, DMSO-d₆): 8.56 (d, 1H), 8.34 (d, 1H), 8.01 (m, 1H), 7.98 (d, 1H), 7.94 (m, 1H), 7.80 (d, 1H), 7.76 (m, 1H), 7.68 (m, 1H), 7.62 (dd, 1H), 7.55 (m, 1H), 7.39 (d, 1H), 6.97 (m, 2H), 5.30 (s, 2H), 4.63 (m, 2H), 4.43 (m, 2H), 3.03 (s, 3H), 2.98 (s, 3H); MS (EI) for C₂₇H₂₅N₃O₂: 424 (MH⁺).

4-quinolin-4-yl-7-quinoxalin-6-yl-2,3,4,5-tetrahydro-1,4-benzoxazepine. Prepared as the trifluoroacetate salt according to the method of example 7 by using quinoxalin-6-ylboronic acid in step 2. ¹H NMR (400 MHz, d₆-DMSO): 8.99 (d, 2H), 8.57 (d, 1H), 8.46 (s, 1H), 8.37-8.29 (m, 2H), 8.27-8.20 (m, 2H), 8.01-7.92 (m, 2H), 7.84 (d, 1H), 7.69 (t, 1H), 7.06 (d, 1H), 7.01 (d, 1H), 5.35 (s, 2H), 4.67 (t, 2H), 4.45 (t, 2H); MS (EI) for C₂₆H₂₀N₄O: 405 (MH⁺).

N-(4-{4-[2-methyl-7-(methyloxy)quinazolin-4-yl]-2,3,4,5-tetrahydro-1,4-benzoxazepin-7-yl}phenyl)acetamide. Synthesized according to the method of example 7 using 4-chloro-7-methoxy-2-methylquinazoline in step 1 and 4-acetamidophenylboronic acid in step 2. ¹H NMR (400 MHz, DMSO-d₆): 10.0 (s, 1H), 7.99 (s, 2H), 7.89 (d, 1H), 7.60 (s, 1H), 7.50 (dd, 1H), 7.40 (t, 1H), 7.31 (m, 1H), 7.11 (m, 2H), 7.03 (d, 1H), 4.97 (s, 2H), 4.44 (s, 2H), 4.15 (s, 2H), 3.88 (s, 3H), 2.42 (s, 3H); MS (EI) for C₂₇H₂₆N₄O₃: 455.2 (MH⁺).

1-methyl-3-(4-{4-[2-methyl-7-(methyloxy)quinazolin-4-yl]-2,3,4,5-tetrahydro-1,4-benzoxazepin-7-yl}phenyl)urea. Prepared according to the method of example 7 by using 4-chloro-7-methoxy-2-methylquinazoline in step 1 and 1-methyl-3-(4-(4,4,5,5-tetramethyl-1,3,2-dioxaborolan-2-yl)phenyl)urea in step 2. ¹H NMR (400 MHz, DMSO-D6); δ 8.62 (s, 1H), 7.89 (d, 1H), 7.64-7.36 (m, 6H), 7.24-6.95 (m, 3H), 6.11-6.03 (m, 1H), 4.99 (s, 2H), 4.47-4.37 (m, 2H), 4.18-4.09 (m, 2H), 3.88 (s, 3H), 2.70-2.60 (m, 3H), 2.43 (s, 3H); MS (EI) for C₂₇H₂₇N₅O₃: 470 (MH⁺).

5-(4-(2-[(dimethylamino)methyl]-6,6-dimethyl-5,6,7,8-tetrahydroquinazolin-4-yl)-2,3,4,5-tetrahydro-1,4-benzoxazepin-7-yl)pyridin-2-amine. Prepared according to the method of example 7 by using 1-(4-chloro-6,6-dimethyl-5,6,7,8-tetrahydroquinazolin-2-yl)-N,N-dimethylmethanamine (reagent preparation 17) in step 1 and 5-(4,4,5,5-tetramethyl-1,3,2-dioxaborolan-2-yl)pyridin-2-amine in step 2. ¹H NMR (400 MHz, DMSO-d₆): 8.22 (d, 1H), 7.68-7.63 (m, 1H), 7.55 (d, 1H), 7.39-7.32 (m, 1H), 6.96 (d, 1H), 6.50 (d, 1H), 6.02 (s, 2H), 4.58 (s, 2H), 4.26 (m, 2H), 3.85 (m, 2H), 3.39 (m, 2H), 2.69 (m, 2H), 2.44 (s, 2H), 2.17 (s, 6H), 1.59 (t, 2H), 0.85 (s, 6H); MS (EI) for C₂₇H₃₄N₆O: 459 (MH⁺).

2-chloro-5-(4-{2-[(dimethylamino)methyl]-6,6-dimethyl-5,6,7,8-tetrahydroquinazolin-4-yl}-2,3,4,5-tetrahydro-1,4-benzoxazepin-7-yl)benzamide. Prepared according to the method of example 7 by using 1-(4-chloro-6,6-dimethyl-5,6,7,8-tetrahydroquinazolin-2-yl)-N,N-dimethylmethanamine (reagent preparation 17) in step 1 and 2-chloro-5-(4,4,5,5-tetramethyl-1,3,2-dioxaborolan-2-yl)benzamide in step 2. ¹H NMR (400 MHz, DMSO-d₆): 7.95 (s, 1H), 7.73-7.64 (m, 4H), 7.57-7.49 (m, 2H), 7.01 (d, 1H), 4.64 (s, 2H), 4.31 (m, 2H), 3.86 (m, 2H), 3.38 (m, 2H), 2.69 (t, 2H), 2.43 (s, 2H), 2.14 (s, 6H), 1.59 (t, 2H), 0.85 (s, 6H); MS (EI) for C₂₉H₃₄ClN₅O₂: 520 (MH⁺).

3-(4-{2-[(dimethylamino)methyl]-6,6-dimethyl-5,6,7,8-tetrahydroquinazolin-4-yl}-2,3,4,5-tetrahydro-1,4-benzoxazepin-7-yl)benzamide. Prepared according to the method of example 7 by using 1-(4-chloro-6,6-dimethyl-5,6,7,8-tetrahydroquinazolin-2-yl)-N,N-dimethylmethanamine (reagent preparation 17) in step 1 and 3-(4,4,5,5-tetramethyl-1,3,2-dioxaborolan-2-yl)benzamide in step 2. ¹H NMR (400 MHz, DMSO-d₆): 8.14 (m, 1H), 8.08 (s, 1H), 7.84-7.71 (m, 3H), 7.58-7.49 (m, 2H), 7.44 (s, 1H), 7.03 (d, 1H), 4.65 (s, 2H), 4.32 (m, 2H), 3.87 (m, 2H), 2.69 (t, 2H), 2.44 (s, 2H), 2.15 (s, 6H), 1.59 (t, 2H), 0.85 (s, 6H); MS (EI) for C₂₉H₃₅N₅O₂: 486 (MH⁺).

1-(4-{7-[4-chloro-3-(methyloxy)phenyl]-2,3-dihydro-1,4-benzoxazepin-4(5H)-yl}-6,6-dimethyl-5,6,7,8-tetrahydroquinazolin-2-yl)-N,N-dimethylmethanamine. Prepared according to the method of example 7 by using 1-(4-chloro-6,6-dimethyl-5,6,7,8-tetrahydroquinazolin-2-yl)-N,N-dimethylmethanamine (reagent preparation 17) in step 1 and 2-(4-chloro-3-methoxyphenyl)-4,4,5,5-tetramethyl-1,3,2-dioxaborolane in step 2. ¹H NMR (400 MHz, Methanol-d₄): 7.60 (s, 1H), 7.49-7.43 (m, 1H), 7.39 (d, 1H), 7.23 (d, 1H), 7.17-7.12 (m, 1H), 7.02 (d, 1H), 4.75 (s, 2H), 4.34 (t, 2H), 4.00 (m, 2H), 3.95 (s, 3H), 3.55 (s, 2H), 2.79 (t, 2H), 2.49 (s, 2H), 2.31 (s, 6H), 1.67 (t, 2H), 0.90 (s, 6H); MS (EI) for C₂₉H₃₅ClN₄O₂: 507 (MH⁺).

### Example 8

### 5-(4-{5-[(4-Fluorophenyl)methyl]-6-methylpyrimidin-4-yl}-2,3,4,5-tetrahydro-1,4-benzoxazepin-7-yl)[1,3]thiazolo[5,4-b]pyridin-2-amine

STEP 1: (4-{[(1,1-dimethylethyl)oxycarbonyl}-2,3,4,5-tetrahydro-1,4-benzoxazepin-7-yl)boronic acid (Example 1, step 2) (1.07 g, 3.64 mmol) was dissolved into 4M hydrogen chloride in dioxane and the resulting solution was allowed to stir at room temperature for 1.3 h. The heterogeneous mixture was then diluted with ethyl ether (100 mL) and the solid collected by filtration to give 2,3,4,5-tetrahydro-1,4-benzoxazepin-7-ylboronic acid hydrochloride salt (791 mg, 95%). ¹H NMR (400 MHz, D₂O): 7.79 (dd, 1H), 7.74 (d, 1H), 7.21 (d, 1H), 4.47 (s, 2H), 4.36 (m, 2H), 3.69 (m, 2H).

STEP 2: A mixture of 2,3,4,5-tetrahydro-1,4-benzoxazepin-7-ylboronic acid hydrochloride salt (1.20 g, 5.2 mmol), 4-chloro-5-(4-fluorobenzyl)-6-methylpyrimidine (reagent preparation 5) (1.24 g, 5.2 mmol), diisopropylethylamine (0.40 mL, 2.10 mmol) 50% aqueous 1,4-dioxane (50 mL) was deoxygenated for five minutes by bubbling nitrogen gas then stirred at 95° C. for 18 hours. The reaction mixture was concentrated, diluted with water (50 mL), adjusted to pH 14, and then washed with isopropyl acetate (3×30 mL). The aqueous mixture was adjusted to pH 8 and the white solid precipitated was collected by filtration, washed with water and dried to give (4-{5-[(4-fluorophenyl)methyl]-6-methylpyrimidin-4-yl}-2,3,4,5-tetrahydro-1,4-benzoxazepin-7-yl)boronic acid (0.72 g, 35% yield). MS (EI) for C₂H₂₁BFN₃O₃: 394 (MH⁺).

STEP 3: A solution of N-(5-bromothiazolo[5,4-b]pyridin-2-yl)benzamide (reagent preparation 13) (0.14 g, 0.42 mmol), (4-{5-[(4-fluorophenyl)methyl]-6-methylpyrimidin-4-yl}-2,3,4,5-tetrahydro-1,4-benzoxazepin-7-yl)boronic acid (0.14 g, 0.35 mmol) and diisopropyethylamine (0.40 mL, 2.10 mmol) in 10% aqueous dimethylformamide (3 mL) was deoxygenated for five minutes by bubbling nitrogen gas, followed by the addition of 1,1′-bis(diphenylphosphino)ferrocenedichloropalladium(II) complex with dichloromethane (20 mg, 0.021 mmol). The reaction mixture was heated to 105° C. for 4 hours. On cooling to room temperature the mixture was diluted with ethyl acetate (50 mL) then it was filtered through a pad of Celite. The organic filtrate was washed with 10% aqueous citric acid (20 mL), brine, aqueous saturated sodium bicarbonate (20 mL) and brine then dried over anhydrous sodium sulfate, filtered and concentrated. Silica gel chromatography (chloroform/methanol 95:5 to 9:1) provided N-[5-(4-{5-[(4-fluorophenyl)methyl]-6-methylpyrimidin-4-yl}-2,3,4,5-tetrahydro-1,4-benzoxazepin-7-yl)[1,3]thiazolo[5,4-b]pyridin-2-yl]benzamide (86 mg. 41%). MS (EI) for C₃₄H₂₇FN₆O₂S: 603 (MH⁺).

STEP 4: A solution of N-[5-(4-{5-[(4-fluorophenyl)methyl]-6-methylpyrimidin-4-yl}-2,3,4,5-tetrahydro-1,4-benzoxazepin-7-yl)[1,3]thiazolo[5,4-b]pyridin-2-yl]benzamide (84 mg, 0.14 mmol) in 70% aqueous sulfuric acid (2 mL) was heated to reflux for thirty minutes. On cooling to room temperature the pH of the mixture was adjusted to −5 by the addition of 50% aqueous sodium hydroxide. Concentration and purification by preparative reverse phase HPLC (0.1% aqueous ammonium acetate-acetonitrile) provided 5-(4-{5-[(4-fluorophenyl)methyl]-6-methylpyrimidin-4-yl}-2,3,4,5-tetrahydro-1,4-benzoxazepin-7-yl)[1,3]thiazolo[5,4-b]pyridin-2-amine (26 mg, 37%). ¹H NMR (400 MHz, d₆-DMSO): 8.44 (d, 1H), 8.36 (s, 1H), 8.08 (d, 1H), 8.01 (br s, 2H), 7.52 (dd, 1H), 7.16-7.10 (m, 4H), 7.00 (d, 1H), 6.90 (d, 1H), 4.48 (s, 2H), 4.28 (m, 2H), 3.96 (s, 2H), 3.76 (m, 2H), 2.14 (s, 3H). MS (EI) for C₂₇H₂₃FN₆OS: 499 (MH⁺).

Using analogous synthetic techniques and substituting with alternative starting reagents in step 3 and conducting protecting group removal step 4 as required according to literature techniques appropriate for a given protecting group (see for example: Greene and Wuts, Protective Groups in Organic Synthetic, Wiley-Interscience) the following compounds of the invention were prepared. Alternative starting materials were obtained commercially unless otherwise indicated.

4-{5-[(4-fluorophenyl)methyl]-6-methylpyrimidin-4-yl}-7-(1H-imidazo[4,5-b]pyridin-6-yl)-2,3,4,5-tetrahydro-1,4-benzoxazepine. Synthesized according to the method of example 8 using 6-bromo-1-trityl-1H-imidazo[4,5-b]pyridine (reagent preparation 15) in step 3. ¹H NMR (400 MHz, d₆-DMSO); 8.49 (m, 3H), 7.54 (d, 1H), 7.08 (m, 5H), 6.99 (m, 1H), 4.52 (s, 2H), 4.24 (br s, 2H), 3.96 (s, 2H), 3.77 (br s, 2H), 2.16 (s, 3H); MS (EI) for C₂₇H₂₃FN₆O: 467 (MH⁺).

5-(4-{5-[(4-fluorophenyl)methyl]-6-methylpyrimidin-4-yl}-2,3,4,5-tetrahydro-1,4-benzoxazepin-7-yl)[1,3]thiazolo[4,5-b]pyridin-2-amine. Synthesized according to the method of example 8 using N-(6-bromothiazolo[4,5-b]pyridin-2-yl)benzamide (J. of Heterocyclic Chemistry 2003, 40(2), 261-268) in step 3. ¹H NMR (400 MHz, d₆-DMSO): 8.51 (s, 1H), 8.37 (d, 1H), 8.10 (d, 1H), 8.02 (br s, 2H), 7.49 (dd, 1H), 7.13-7.08 (m, 4H), 7.02 (d, 1H), 6.88 (d, 1H), 4.50 (s, 2H), 4.29 (m, 2H), 3.98 (s, 2H), 3.78 (m, 2H), 2.16 (s, 3H). MS (EI) for C₂₇H₂₃N₆O₂: 499 (MH⁺).

6-(4-{5-[(4-fluorophenyl)methyl]-6-methylpyrimidin-4-yl}-2,3,4,5-tetrahydro-1,4-benzoxazepin-7-yl)quinazolin-2-amine. Synthesized according to the method of example 8 using 6-bromoquinazolin-2-amine in step 3. MS (EI) for C₂₉H₂₅FN₆O: 493 (MH⁺).

6-(4-{5-[(4-fluorophenyl)methyl]-6-methylpyrimidin-4-yl}-2,3,4,5-tetrahydro-1,4-benzoxazepin-7-yl)[1,2,4]triazolo[1,5-a]pyridin-2-amine. Prepared according to the method of example 8 by using bis(1,1-dimethylethyl)(6-bromo[1,2,4]triazolo[1,5-a]pyridine-2-yl)imidodicarbonate (reagent preparation 14) in step 3. ¹H NMR (400 MHz, DMSO-d₆): 8.69 (m, 1H), 8.48 (s, 1H), 7.51 (m, 2H), 7.40 (d, 1H), 7.10 (d, 4H), 6.99 (d, 1H), 6.91 (m, 1H), 6.06 (s, 2H), 4.47 (s, 2H), 4.26 (m, 2H), 3.95 (s, 2H), 3.74 (m, 2H), 2.14 (s, 3H); MS (EI) for C₂₇H₂₄FN₇O: 482 (MH⁺).

6-(4-{5-[(4-fluorophenyl)methyl]-6-methylpyrimidin-4-yl}-2,3,4,5-tetrahydro-1,4-benzoxazepin-7-yl)imidazo[1,2-a]pyridin-2-amine. Prepared according to the method of example 8 by using N-(6-bromoimidazo[1,2-a]pyridin-2-yl)-2,2,2-trifluoroacetamide (Tetrahedron Letters 2002, 43(50), 9051-9054) in step 3. ¹H NMR (400 MHz, methanol-d₄): 8.47 (s, 1H), 8.25 (s, 1H), 7.41 (dd, 1H), 7.32 (d, 1H), 7.24 (dd, 1H), 7.10 (m, 2H), 7.00 (m, 3H), 6.71 (m, 1H), 4.53 (s, 2H), 4.29 (m, 2H), 3.98 (s, 2H), 3.88 (m, 2H), 2.21 (s, 3H); MS (EI) for C₂₈H₂₅FN₆O: 481 (MH⁺).

5-(4-{5-[(4-fluorophenyl)methyl]-6-methylpyrimidin-4-yl}-2,3,4,5-tetrahydro-1,4-benzoxazepin-7-yl)-1,3-benzothiazol-2-amine. Prepared according to the method of example 8 by using N-(5-bromobenzo[d]thiazol-2-yl)acetamide (Journal of the Indian Chemical Society (1958), 35 807-10) in step 3. ¹H NMR (400 MHz, methanol-d₄): 8.47 (s, 1H), 7.62 (d, 1H), 7.46 (d, 1H), 7.43 (dd, 1H), 7.08 (m, 3H), 6.99 (m, 3H), 6.81 (d, 1H), 4.53 (s, 2H), 4.29 (m, 2H), 4.00 (s, 2H), 3.89 (m, 2H), 2.22 (s, 3H); MS (EI) for C₂H₂₄FN₅OS: 498 (MH⁺).

6-(4-{5-[(4-fluorophenyl)methyl]-6-methylpyrimidin-4-yl}-2,3,4,5-tetrahydro-1,4-benzoxazepin-7-yl)imidazo[1,2-a]pyrimidin-2-amine. Synthesized according to the method of example 8 using N-(6-bromoimidazo[1,2-a]pyrimidin-2-yl)-2,2,2-trifluoroacetamide (Synthesis 1999, 12, 2124-2130) in step 3. ¹H NMR (400 DMSO-D₆): 8.91 (s, 1H), 8.61 (br, 1H), 8.46 (s, 1H), 8.33 (br, 1H), 7.44 (dd, 1H), 7.13 to 6.98 (m, 5H), 6.78 (br, 1H), 4.56 (s, 2H), 4.29 (m, 2H), 4.00 (s, 2H), 3.88 (m, 2H), 2.22 (s, 3H), MS (EI) for C₂₇H₂₄FN₇O: 482 (MH⁺).

7-(4-{5-[(4-fluorophenyl)methyl]-6-methylpyrimidin-4-yl}-2,3,4,5-tetrahydro-1,4-benzoxazepin-7-yl)[1,2,4]triazolo[1,5-a]pyridin-2-amine. Prepared according to the method of example 8 by using N-(7-bromo-[1,2,4]triazolo[1,5-a]pyridin-2-yl)acetamide (reagent preparation 16) in step 3. ¹H NMR (400 MHz, methanol-d₄): 8.47-8.42 (m, 2H), 7.55 (d, 1H), 7.40 (s, 1H), 7.12-6.99 (m, 6H), 6.91 (s, 1H), 4.58 (s, 2H), 4.32 (t, 2H), 4.00 (s, 2H), 3.89 (t, 2H), 2.22 (s, 3H); MS (EI) for C₂₇H₂₄FN₇O: 482 (MH⁺).

6-(4-{5-[(4-fluorophenyl)methyl]-6-methylpyrimidin-4-yl}-2,3,4,5-tetrahydro-1,4-benzoxazepin-7-yl)-1H-imidazo[4,5-c]pyridin-2-amine. Prepared according to the method of example 8 by using methyl (6-bromo-1H-imidazo[4,5-c]pyridine-2-yl)carbamate (reagent preparation 45) in step 3. ¹H NMR (400 MHz, methanol-d₄): 8.45 (s, 1H), 8.35 (s, 1H), 7.62 (m, 1H), 7.72 (m, 1H), 7.41 (s, 1H), 7.04 (m, 4H), 6.93 (m, 2H), 4.57 (s, 2H), 4.32 (m, 2H), 3.97 (s, 2H), 3.88 (m, 2H), 2.21 (s, 3H), 1.96 (s, 3H); MS (EI) for C₂₇H₂₄FN₇O: 482 (MH⁺).

2-amino-5-(4-{5-[(4-fluorophenyl)methyl]-6-methylpyrimidin-4-yl}-2,3,4,5-tetrahydro-1,4-benzoxazepin-7-yl)-N-methylpyridine-3-sulfonamide. Prepared according to the method of example 8 by using 2-amino-5-bromo-N-methylpyridine-3-sulfonamide (WO 2008144463) in step 3. ¹H NMR (400 MHz, DMSO-d₆): 8.48 (s, 1H), 8.36 (m, 1H), 7.93 (m, 1H), 7.71 (br. s, 1H), 7.41 (m, 1H), 7.09 (m, 4H), 7.00 (d, 1H), 6.92 (m, 1H), 6.70 (br. s, 1H), 4.50 (s, 2H), 4.26 (m, 2H), 3.96 (s, 2H), 3.75 (m, 2H), 2.45 (s, 3H), 2.15 (s, 3H); MS (EI) for C₂₇H₂₇FN₆O₃S: 535 (MH⁺).

6-(4-{5-[(4-fluorophenyl)methyl]-6-methylpyrimidin-4-yl}-2,3,4,5-tetrahydro-1,4-benzoxazepin-7-yl)[1,3]thiazolo[5,4-b]pyridin-2-amine. Synthesized according to the method of example 8 using N-(6-bromothiazolo[5,4-b]pyridin-2-yl)acetamide (J. Heterocyclic Chemistry 2003, 40, 261) in step 3. ¹H NMR (400 MHz, d₆-DMSO): 8.47 (s, 1H), 8.20 (d, 1H), 7.88 (s, 2H), 7.69 (d, 1H), 7.54 (dd, 1H), 7.09 (d, 4H), 6.99 (d, 1H), 6.94 (d, 1H), 4.45 (s, 2H), 4.24 (m, 2H), 3.97 (s, 2H), 3.77 (m, 2H), 2.14 (s, 3H); MS (EI) for C₂₇H₂₃FN₆OS: 499 (MH⁺).

N-ethyl-6-(4-{5-[(4-fluorophenyl)methyl]-6-methylpyrimidin-4-yl}-2,3,4,5-tetrahydro-1,4-benzoxazepin-7-yl)-1H-imidazo[4,5-b]pyridin-2-amine. Prepared as in example 8 using 6-bromo-N-ethyl-N,3-bis(methoxymethyl)-3H-imidazo[4,5-b]pyridin-2-amine (reagent preparation 36) in step 3. ¹H NMR (400 MHz, d₆-DMSO) δ 11.04 (s, 1H), 8.49 (s, 1H), 8.04 (s, 1H), 7.89 (s, 1H), 7.52-7.35 (m, 2H), 7.10 (d, 4H), 7.00 (d, 1H), 6.91 (d, 1H), 4.48 (s, 2H), 4.26 (t, 2H), 4.00 (s, 2H), 3.77 (t, 2H), 3.33 (q, 2H), 2.16 (s, 3H), 1.20 (q, 3H); MS (ES) for C₂₉H₂₈FN₇O: 510.2 (MH⁺).

5-(4-{5-[(4-fluorophenyl)methyl]-6-methylpyrimidin-4-yl}-2,3,4,5-tetrahydro-1,4-benzoxazepin-7-yl)-1H-indazol-3-amine. Prepared according to example 8 using tert-butyl 3-(bis(tert-butoxycarbonyl)amino)-5-bromo-1H-indazole-1-carboxylate (reagent preparation 48) in step 1 and BOC group deprotection. ¹H NMR (400 MHz, d₆-DMSO) δ 11.44 (s, 1H), 8.50 (s, 1H), 7.86 (s, 1H), 7.42 (dd, 1H), 7.28 (s, 2H), 7.19-7.07 (m, 4H), 7.01 (d, 1H), 6.87 (s, 1H), 5.42 (s, 2H), 4.49 (s, 2H), 4.26 (t, 2H), 4.00 (s, 2H), 3.77 (t, 2H), 2.17 (s, 3H).; MS (EI) for C₂₈H₂₅FN₆O: 481 (MH⁺).

### Example 9

### 5-(4-{5-[(4-fluorophenyl)methyl]-6-methylpyrimidin-4-yl}-2,3,4,5-tetrahydro-1,4-benzoxazepin-7-yl)pyrazin-2-amine

STEP 1: 1,1-Dimethylethyl 7-bromo-2,3-dihydro-1,4-benzoxazepine-4(5H)-carboxylate (5.0 g, 20.1 mmol), bis(pinacolato)diboron (5.6 g, 22.1 mmol), potassium acetate (5.9 g, 60.2 mmol) and [1,1′-bis(diphenylphosphino)ferrocene]dichloropalladium(II) (440 mg, 0.62 mmol) were heated in DMSO (5 mL) solution at 80 C for 1.5 h. The mixture was then cooled to room temperature and diluted with an excess of ethyl acetate and filtered through a bed of celite. The filtrate was partitioned with 1M aqueous hydrochloric acid and the organic phase washed with brine and dried over anhydrous sodium sulfate. The mixture was filtered and concentrated and the residue purified by silica chromatography using 4:1 hexanes:ethyl acetate as eluent to give 1,1-dimethylethyl 7-(4,4,5,5-tetramethyl-1,3,2-dioxaborolan-2-yl)-2,3-dihydro-1,4-benzoxazepine-4(5H)-carboxylate (7.6 g, 100%). ¹H NMR (400 MHz, CDCl₃): 7.77 (s, 0.4H), 7.67 (s, 1H), 7.65 (s, 0.6H), 7.04-6.98 (m, 1H), 4.54 (s, 0.7H), 4.43 (s, 1.3H), 4.09-4.01 (m, 2H), 3.79 (dd, 2H), 1.40 (br s, 9H), 1.26 (s, 12H). MS (EI) for C₂₀H₃₀BNO₅: 376 (MH⁺).

STEP 2: A solution of 1,1-dimethylethyl 7-(4,4,5,5-tetramethyl-1,3,2-dioxaborolan-2-yl)-2,3-dihydro-1,4-benzoxazepine-4(5H)-carboxylate (3.0 g, 8.00 mmol) in dichloromethane (90 mL) and trifluoroacetic acid (10 mL) was heated to reflux for 1 h, and then cooled to room temperature. The reaction mixture was concentrated and the residue was azeotroped with toluene (100 mL) to give 7-(4,4,5,5-tetramethyl-1,3,2-dioxaborolan-2-yl)-2,3-dihydro-1,4-benzoxazepine trifluoroacetate salt (2.9 g, quantitative yield). MS (EI) for C₁₅H₂₂BNO₃: 276 (MH⁺).

STEP 3: A mixture of 7-(4,4,5,5-tetramethyl-1,3,2-dioxaborolan-2-yl)-2,3-dihydro-1,4-benzoxazepine trifluoroacetate salt (2.9 g, 8.00 mmol, 4-chloro-5-[(4-fluorophenyl)methyl]-6-methylpyrimidine (reagent preparation 5) (1.9 g, 8.00 mmol) and N,N-diisopropylethylamine (7.0 mL, 40.0 mmol) in N-methyl-2-pyrrolidone (10 mL) was reacted in a microwave apparatus (250 W) for 2 h at 150° C. After cooling to room temperature the reaction mixture was partitioned between ethyl acetate (500 mL) and brine (100 mL). The organic layer was separated, washed with brine (100 mL), dried over sodium sulfate then filtered and concentrated. Column chromatography of the residue on silica (gradient 20 to 40% ethyl acetate in hexane) gave 4-(5-[(4-fluorophenyl)methyl]-6-methylpyrimidin-4-yl)-7-(4,4,5,5-tetramethyl-1,3,2-dioxaborolan-2-yl)-2,3,4,5-tetrahydro-1,4-benzoxazepine (1.6 g, 42% yield). ¹H NMR (400 MHz, DMSO-D₆); 8.58 (s, 1H), 7.62 (dd, 1H), 7.08 (m, 4H), 7.02 (d, 1H), 6.96 (d, 1H), 4.36 (s, 2H), 4.30 (m, 2H), 3.92 (s, 2H), 3.84 (m, 2H), 2.26 (s, 3H), 1.36 (s, 12H); MS (EI) for C₂₇H₃₁BFN₃O₃: 476 (MH⁺).

STEP 4: A solution of 4-{5-[(4-fluorophenyl)methyl]-6-methylpyrimidin-4-yl}-7-(4,4,5,5-tetramethyl-1,3,2-dioxaborolan-2-yl)-2,3,4,5-tetrahydro-1,4-benzoxazepine (0.10 g, 0.21 mmol), 2-amino-5-bromopyrazine (40 mg, 0.21 mmol) and potassium carbonate (0.12 g, 0.84 mmol) in N,N-dimethylformamide (5 mL), and water (0.5 mL) was degassed by bubbling nitrogen gas for five minutes followed by the addition of [1,1′-bis(diphenylphosphino)ferrocene]dichloropalladium(II) (15 mg, 0.021 mmol), then stirred at 95° C. for 16 hours. After cooling to room temperature the reaction mixture was partitioned between ethyl acetate (50 mL) and brine (30 mL). The organic layer was separated, washed with brine, dried over sodium sulfate then filtered and concentrated. Purification by preparative reverse phase HPLC (0.1% aqueous ammonium acetate-acetonitrile) provided 5-(4-{5-[(4-fluorophenyl)methyl]-6-methylpyrimidin-4-yl}-2,3,4,5-tetrahydro-1,4-benzoxazepin-7-yl)pyrazin-2-amine (18 mg, 20%). ¹H NMR (400 MHz, d₆-DMSO): 8.80 (s, 1H), 8.38 (s, 1H), 7.96 (s, 1H), 7.72 (d, 1H), 7.50 (br s, 1H), 7.25-7.14 (m, 4H), 6.93 (d, 1H), 4.90 (s, 2H), 4.32 (m, 2H), 4.00 (m, 4H), 2.22 (s, 3H). MS (EI) for C₂₅H₂₃FNO₆: 443 (MH⁺).

Using analogous synthetic techniques and substituting with alternative starting reagents in steps 3 or 4 the following compounds of the invention were prepared. Alternative starting materials were obtained commercially unless otherwise indicated.

6-(4-{5-[(4-fluorophenyl)methyl]-6-methylpyrimidin-4-yl}-2,3,4,5-tetrahydro-1,4-benzoxazepin-7-yl)pyridazin-3-amine. Synthesized according to the method of example 9 using 6-bromopyridazin-3-amine in step 4. ¹H NMR (400 MHz, d₆-DMSO): 8.46 (s, 1H), 7.74 (dd, 1H), 7.54 (d, 1H), 7.28 (d, 1H), 7.18-7.06 (m, 4H), 6.98 (d, 1H), 6.82 (d, 1H), 6.47 (s, 2H), 4.51 (s, 2H), 4.29) m, 2H), 3.95 (s, 2H), 3.75 (m, 2H), 2.16 (s, 3H). MS (EI) for C₂₅H₂₃FN₆O: 463 (MH⁺).

5-(4-{5-[(4-fluorophenyl)methyl]-6-methylpyrimidin-4-yl}-2,3,4,5-tetrahydro-1,4-benzoxazepin-7-yl)pyrimidin-2-amine. Synthesized according to the method of example 9 using 5-bromopyrimidin-2-amine in step 4. ¹H NMR (400 MHz, DMSO-D₆): 8.49 (s, 1H), 8.37 (s, 2H), 7.42 (d, 1H), 7.09 (d, 4 h), 6.98 (d, 1H), 6.76-6.83 (m, 3H), 4.46 (s, 2H), 4.25-4.27 (m, 2H), 3.96 (s, 2H), 3.74-3.76 (m, 2H), 2.15 (s; 3H); MS (EI) for C₂₅H₂₃FN₆O: 443 (MH⁺).

6-(4-{5-[(4-fluorophenyl)methyl]-6-methylpyrimidin-4-yl}-2,3,4,5-tetrahydro-1,4-benzoxazepin-7-yl)-N-methylpyridine-3-carboxamide. Synthesized according to the method of example 9 using 6-bromo-N-methylnicotinamide in step 4. ¹H NMR (400 MHz, DMSO-D₆): 9.05 (s, 1H), 8.65-8.72 (m, 2H), 8.47 (s, 1H), 8.23 (dd, 1 h), 7.95 (dd, 1H), 7.83 (d, 1H), 7.45 (s, 1H), 7.02-7.18 (m, 5H), 4.54 (s, 2H), 4.31-4.38 (m, 2H), 3.94 (s, 2H), 3.75-3.81 (m, 2H), 2.84 (s, 3H), 2.15 (s; 3H); MS (EI) for C₂₈H₂₆FN₅O₂: 484 (MH⁺).

4-{5-[(4-fluorophenyl)methyl]-6-methylpyrimidin-4-yl}-7-(1,3-thiazol-5-yl)-2,3,4,5-tetrahydro-1,4-benzoxazepine. Synthesized according to the method of example 9 using 5-bromothiazole in step 4. ¹H NMR (400 MHz, d₆-DMSO): 9.05 (s, 1H), 8.48 (s, 1H), 8.08 (s, 1H), 7.51 (dd, 1H), 7.18-7.06 (m, 4H), 6.99 (d, 1H), 6.90 (d, 1H), 4.49 (s, 2H), 4.29 (m, 2H), 3.92 (s, 2H), 3.72 (m 2H), 2.14 (s, 3H). MS (EI) for C₂₄H₂₁FN₄OS: 433 (MH⁺).

4-(7,7-dimethyl-5,6,7,8-tetrahydroquinazolin-4-yl)-7-(2-methyl-1,3-thiazol-5-yl)-2,3,4,5-tetrahydro-1,4-benzoxazepine. Synthesized according to the method of example 9 using 4-chloro-7,7-dimethyl-5,6,7,8-tetrahydroquinazoline (reagent preparation 3) in step 3 and 5-bromo-2-methylthiazole in step 4. ¹H NMR (400 MHz, d₆-DMSO): 8.32 (s, 1H), 7.91 (s, 1H), 7.53 (d, 1H), 7.40 (dd, 1H), 7.08 (d, 1H), 4.72 (s, 2H), 4.34 (m, 2H), 3.90 (m, 2H), 2.68 (m ands, 5H), 2.46 (s, 2H), 1.46 (t, 2H), 1.00 (6H). MS (EI) for C₂₃H₂₆N₄OS: 407 (MH⁺).

5-(4-{5-[(4-fluorophenyl)methyl]-6-methylpyrimidin-4-yl}-2,3,4,5-tetrahydro-1,4-benzoxazepin-7-yl)-4-methyl-1,3-thiazol-2-amine. Synthesized according to the method of example 9 using bis(1,1-dimethylethyl) (5-bromo-4-methyl-1,3-thiazol-2-yl)imidodicarbonate (reagent preparation 14) in step 4. ¹H NMR (400 MHz, d₆-DMSO): 8.46 (s, 1H), 7.13-7.02 (m, 5H), 6.94 (br s, 2H), 6.91 (s, 1H), 6.68 (d, 1H), 4.42 (s, 2H), 4.22 (m, 2H), 4.14 (s, 2H), 3.73 (m, 2H), 2.14 (s, 3H), 2.06 (s, 3H). MS (EI) for C₂₅H₂₄FN₅OS: 462 (MH⁺).

5-[4-(7,7-dimethyl-5,6,7,8-tetrahydroquinazolin-4-yl)-2,3,4,5-tetrahydro-1,4-benzoxazepin-7-yl]-4-methyl-1,3-thiazol-2-amine. Synthesized according to the method of example 9 using 4-chloro-7,7-dimethyl-5,6,7,8-tetrahydroquinazoline (reagent preparation 3) in step 3 and bis(1,1-dimethylethyl) (5-bromo-4-methyl-1,3-thiazol-2-yl)imidodicarbonate (reagent preparation 14) in step 4. ¹H NMR (400 MHz, d₆-DMSO): 8.34 (s, 1H), 7.24 (d, 1H), 7.11 (dd, 1H), 6.96 (d, 1H), 6.92 (s, 2H), 4.68 (s, 2H), 4.30 (m, 2H), 3.89 (m, 2H), 2.62 (t, 2H), 2.44 (s, 2H), 2.12 (s, 3H), 1.42 (t, 2H), 1.00 (s, 6H). MS (EI) for C₂₃H₂₇N₅OS: 422 (MH⁺).

### Example 10

### N,N-dimethyl-3-({4-[7-(2-methyl-1H-benzimidazol-6-yl)-2,3-dihydro-1,4-benzoxazepin-4(5H)-yl]quinazolin-6-yl}oxy)propan-1-amine and 4-[7-(2-methyl-1H-benzimidazol-6-yl)-2,3-dihydro-1,4-benzoxazepin-4(5H)-yl]quinazolin-6-ol

STEP 1; A solution of 7-(2-methyl-1H-benzimidazol-6-yl)-4-{6-[(phenylmethyl)oxy]quinazolin-4-yl}-2,3,4,5-tetrahydro-1,4-benzoxazepine (example 1) (26 mg, 0.05 mmol) in trifluoroacetic acid (3.0 mL) was heated to 68° C. for 3.5 hours then concentrated and dried. This material was then carried forward into step 2 without further purification. A sample of material obtained in this manner was purified by preparative reverse phase HPLC to give 4-[7-(2-methyl-1H-benzimidazol-6-yl)-2,3-dihydro-1,4-benzoxazepin-4(5H)-yl]quinazolin-6-ol. ¹H NMR (400 MHz, d₆-DMSO): 10.1 (s, 1H), 8.44 (s, 1H), 7.73 (d, 1H), 7.70 (d, 1H), 7.65 (d, 1H), 7.55-7.51 (m, 2H), 7.48 (d, 0.5H), 7.46 (d, 0.5H), 7.40 (dd, 1H), 7.33 (d, 1H), 7.04 (d, 1H), 5.00 (s, 2H), 4.46 (t, 2H), 4.24 (t, 2H), 2.54 (s, 3H). MS (EI) for C₂₅H₂₂N₅O₂: 424 (MH⁺).

STEP 2: 4-[7-(2-Methyl-1H-benzimidazol-6-yl)-2,3-dihydro-1,4-benzoxazepin-4(5H)-yl]quinazolin-6-ol as obtained in step 1 without purification was taken into N,N-dimethylacetamide (5.0 mL), and then combined with 3-chloro-N,N-dimethylpropan-1-amine hydrochloride (9.61 mg, 0.06 mmol) and potassium carbonate (41 mg, 0.30 mmol) and heated to 50° C. for 18 hours. The reaction mixture was concentrated and the residue purified by preparative reverse phase HPLC to give N,N-dimethyl-3-({4-[7-(2-methyl-1H-benzimidazol-6-yl)-2,3-dihydro-1,4-benzoxazepin-4(5H)-yl]quinazolin-6-yl}oxy)propan-1-amine as the trifluoroacetic acid salt (6.5 mg, 21% yield). ¹H NMR (400 MHz, d6-DMSO): 8.69 (s, 1H), 7.97 (s, 2H), 8.23 (m, 3H), 7.61 (dd, 2H), 7.30 (br s, 1H), 7.07 (d, 1H), 5.30 (br s, 2H), 4.63 (br s, 2H), 4.37 (br s, 2H), 3.98 (br, s, 2H), 2.98 (br s, 2H), 2.80 (s, 2H), 2.68 (s, 3H), 2.50 (s, 6H); MS (EI) for C₃₀H₃₂N₆O₂: 509 (MH⁺).

Using analogous synthetic techniques and substituting with alternative starting reagents in steps 1 or 2 the following compounds of the invention were prepared. Alternative starting materials were obtained commercially unless otherwise indicated.

4-[6-(ethyloxy)quinazolin-4-yl]-7-(2-methyl-1H-benzimidazol-6-yl)-2,3,4,5-tetrahydro-1,4-benzoxazepine. Synthesized according to the method of example 10 using iodoethane in step 2. ¹H NMR (400 MHz, d₆-DMSO): 12.22 (s, 1H), 8.53 (s, 1H), 7.84 (d, 1H), 7.79 (m, 0.5H), 7.73 (d, 1H), 7.65 (m, 0.5H), 7.58 (dd, 1H), 7.54 (m, 0.5H), 7.46 (br s, 1H), 7.44 (m, 0.5H), 7.40 (dd, 1H), 7.09 (br s, 1H), 7.05 (d, 1H), 5.06 (s, 2H), 4.54 (t, 2H), 4.08 (t, 2H), 3.72 (dd, 2H), 2.50 (s, 3H), 0.78 (t, 3H). MS (EI) for C₂₇H₂₆N₅O₂: 452 (MH⁺).

({4-[7-(2-methyl-1H-benzimidazol-6-yl)-2,3-dihydro-1,4-benzoxazepin-4(5H)-yl]quinazolin-6-yl}oxy)acetonitrile. Synthesized according to the method of example 10 using bromoacetonitrile in step 2. ¹H NMR (400 MHz, d₆-DMSO): 12.22 (s, 1H), 8.53 (s, 1H), 7.83 (s, 0.5H), 7.81 (s, 0.5H), 7.72 (d, 1H), 7.67 (m, 0.5H), 7.62 (d, 0.5H), 7.59 (d, 0.5H), 7.53 (d, 1H), 7.52 (d, 1H), 7.50 (m, 0.5H), 7.44 (d, 1H), 7.40 (dd, 1H), 7.04 (d, 1H), 5.22 (s, 2H), 5.10 (s, 2H), 4.50 (t, 2H), 4.18 (t, 2H), 2.50 (s, 3H). MS (EI) for C₂₇H₂₃N₆O₂: 463 (MH⁺).

7-(2-methyl-1H-benzimidazol-6-yl)-4-[6-(propyloxy)quinazolin-4-yl]-2,3,4,5-tetrahydro-1,4-benzoxazepine. Synthesized according to the method of example 10 using 1-bromopropane in step 2. ¹H NMR (400 MHz, d₆-DMSO): 8.75 (s, 1H), 7.97 (s, 2H), 7.81 (s, 2H), 7.75 (d, 1H), 7.70 (d, 1H), 7.58 (d, 1H), 7.22 (br s, 1H), 7.11 (d, 1H), 5.27 (s, 2H), 4.62 (br s, 2H) 4.32 (br s, 2H), 3.73 (br s, 2H), 1.33 (br s, 2H), 0.70 (br s, 3H); MS (EI) for C₂₈H₂₇N₅O₂: 466 (MH⁺).

7-(2-methyl-1H-benzimidazol-6-yl)-4-(6-{[2-(methyloxy)ethyl]oxy}quinazolin-4-yl)-2,3,4,5-tetrahydro-1,4-benzoxazepine. Synthesized according to the method of example 10 using 2-(methoxy)-1-bromoethane in step 2. ¹H NMR (400 MHz, d₆-DMSO): 8.52 (s, 1H), 7.88 (d, 1H), 7.58 (dd, 1H), 7.43 (m, 3H), 7.40 (m, 3H), 7.06 (m, 2H), 5.07 (s, 2H), 4.57 (br s, 2H) 4.08 (br s, 2H), 3.75 (br s, 2H), 2.85 (br s, 2H), 2.79 (s, 3H), 1.82 (s, 3H); MS (EI) for C₂₈H₂₇N₅O₃: 482 (MH⁺).

N,N-dimethyl-2-({4-[7-(2-methyl-1H-benzimidazol-6-yl)-2,3-dihydro-1,4-benzoxazepin-4(5H)-yl]-8-(methyloxy)quinazolin-7-yl}oxy)ethanamine. Synthesized according to the method of example 10 using 7-(2-methyl-1H-benzimidazol-6-yl)-4-{8-(methyloxy)-7-[(phenylmethyl)oxy]quinazolin-4-yl}-2,3,4,5-tetrahydro-1,4-benzoxazepine (example 1) in step 1 and 2-(N,N-dimethylamino)-1-chloroethane in step 2. ¹H NMR (400 MHz, d₆-DMSO): 8.57 (s, 1H), 7.93 (s, 1H), 7.82 (m, 3H), 7.59 (d, 1H), 7.51 (d, 1H), 7.01 (d, 1H), 7.01 (d, 1H), 5.38 (br s, 2H), 4.61 (m, 4H), 4.45 (br s, 2H), 3.95 (br s, 2H), 3.72 (t, 2H), 2.90 (s, 6H), 2.77 (s, 3H); MS (EI) for C₃₀H₃₂N₆O₃: 525 (MH⁺).

7-(2-methyl-1H-benzimidazol-6-yl)-4-{8-(methyloxy)-7-[(2-methylpropyl)oxy]quinazolin-4-yl}-2,3,4,5-tetrahydro-1,4-benzoxazepine. Synthesized according to the method of example 10 using 7-(2-methyl-1H-benzimidazol-6-yl)-4-{8-(methyloxy)-7-[(phenylmethyl)oxy]quinazolin-4-yl}-2,3,4,5-tetrahydro-1,4-benzoxazepine (example 1) in step 1 and isobutyl bromide in step 2. ¹H NMR (400 MHz, d₆-DMSO): 8.48 (s, 1H), 7.96 (s, 1H), 7.83 (m, 3H), 7.63 (d, 1H), 7.52 (d, 1H), 7.04 (d, 1H), 5.46 (s, 2H), 4.65 (br s, 3H) 4.53 (br s, 2H), 4.07 (d, 2H), 3.92 (s, 3H), 2.79 (s, 3H), 2.13 (m, 1H), 1.04 (d, 6H); MS (EI) for C₃₀H₃₁N₅O₃: 510 (MH⁺).

4-{7-[(cyclopropylmethyl)oxy]-8-(methyloxy)quinazolin-4-yl}-7-(2-methyl-1H-benzimidazol-6-yl)-2,3,4,5-tetrahydro-1,4-benzoxazepine. Synthesized according to the method of example 10 using 7-(2-methyl-1H-benzimidazol-6-yl)-4-{8-(methyloxy)-7-[(phenylmethyl)oxy]quinazolin-4-yl}-2,3,4,5-tetrahydro-1,4-benzoxazepine (example 1) in step 1 and cyclopropylmethyl bromide in step 2. ¹H NMR (400 MHz, d₆-DMSO): 8.66 (s, 1H), 8.08 (s, 1H), 7.92 (s, 1H), 7.81 (m, 3H), 7.63 (d, 1H), 7.50 (d, 1H), 7.04 (d, 1H), 5.44 (s, 2H), 4.63 (br s, 3H) 4.53 (br s, 2H), 4.17 (d, 2H), 3.92 (s, 3H), 0.83 (m, 1H), 0.61 (m, 2H), 0.41 (m, 2H); MS (EI) for C₃₀H₂₉N₅O₃: 508 (MH⁺).

7-(2-methyl-1H-benzimidazol-6-yl)-4-{8-(methyloxy)-7-[(quinolin-2-ylmethyl)oxy]quinazolin-4-yl}-2,3,4,5-tetrahydro-1,4-benzoxazepine. Synthesized according to the method of example 10 using 7-(2-methyl-1H-benzimidazol-6-yl)-4-{8-(methyloxy)-7-[(phenylmethyl)oxy]quinazolin-4-yl}-2,3,4,5-tetrahydro-1,4-benzoxazepine (example 1) in step 1 and 2-bromomethylquinoline in step 2. ¹H NMR (400 MHz, d₆-DMSO): 8.64 (s, 1H), 8.47 (d, 1H), 8.03 (m, 4H), 7.83 (m, 4H), 7.73 (d, 1H), 7.63 (m, 3H), 7.02 (d, 1H), 5.64 (s, 2H), 5.43 (br s, 3H) 4.62 (br s, 2H), 4.51 (br s, 2H), 4.01 (s, 1H), 2.80 (s, 3H); MS (EI) for C₃₆H₃₀N₆O₃: 595 (MH⁺).

4-[7-(2-methyl-1H-benzimidazol-6-yl)-2,3-dihydro-1,4-benzoxazepin-4(5H)-yl]-8-(methyloxy)quinazolin-7-ol. Synthesized according to the method of example 10 using 7-(2-methyl-1H-benzimidazol-6-yl)-4-{8-(methyloxy)-7-[(phenylmethyl)oxy]quinazolin-4-yl}-2,3,4,5-tetrahydro-1,4-benzoxazepine (example 1) in step 1. ¹H NMR (400 MHz, d₆-DMSO): 12.86 (br s, 1H), 10.29 (br s, 1H), 8.48 (s, 1H), 7.76-7.67 (m, 3H), 7.56 (d, 1H), 7.51 (dd, 1H), 7.46 (dd, 1H), 7.11 (d, 1H), 7.00 (d, 1H), 5.12 (s, 2H), 4.50 (m, 2H), 4.22 (m, 2H), 3.88 (s, 3H), 2.54 (s, 3H). MS (EI) for C₂₆H₂₃N₅O₃: 454 (MH⁺)

4-[7-(2-methyl-1H-benzimidazol-6-yl)-2,3-dihydro-1,4-benzoxazepin-4(5H)-yl]-6-(methyloxy)quinazolin-7-ol. Prepared according to the method of example 10 by using 7-(2-methyl-1H-benzimidazol-6-yl)-4-{6-(methyloxy)-7-[(phenylmethyl)oxy]quinazolin-4-yl}-2,3,4,5-tetrahydro-1,4-benzoxazepine (example 1) in step 1. ¹H NMR (400 MHz, Methanol-D₄): 8.41, (s, 1H), 7.67 (br, 2H), 7. (dd, 2H), 7.45 (dd, 1H), 7.10 (d, 2H), 7.03 (dd, 1H), 5.03 (s, 2H), 4.54 (m, 2H), 4.15 (m, 2H), 3.48 (s, 3H), 2.60 (s, 3H), MS (EI) for C₂₆H₂₃N₅O₃: 454 (MH⁺).

N-ethyl-6-({4-[7-(ethyloxy)-2-methylquinazolin-4-yl]-2,3,4,5-tetrahydro-1,4-benzoxazepin-7-yl}-1H-benzimidazol-2-amine. Prepared according to the method of example 10 using 4-{7-[2-(ethylamino)-1H-benzimidazol-6-yl]-2,3-dihydro-1,4-benzoxazepin-4(5H)-yl}-2-methylquinazolin-7-ol (example 11) and ethyl iodide in step 2. ¹H NMR (400 MHz, DMSO-d6) δ 10.36 (s, 1H), 7.91-7.82 (m, 1H), 7.63 (dd, 1H), 7.52-7.36 (m, 2H), 7.26-7.13 (m, 2H), 6.99 (dd, 1H), 6.94-6.86 (m, 2H), 6.70 (q, 1H), 4.96 (s, 2H), 4.45-4.35 (m, 2H), 4.16-3.98 (m, 4H), 3.46-3.36 (m, 2H), 2.44-2.37 (m, 3H), 1.28-1.17 (m, 6H); MS (EI) for C₂₉H₃₀N₆O₂: 495 (MH⁺).

### Example 11

### N-ethyl-6-{4-[2-methyl-7-(methyloxy)quinazolin-4-yl]-2,3,4,5-tetrahydro-1,4-benzoxazepin-7-yl}-1H-benzimidazol-2-amine

STEP 1: To a solution of 1,1-dimethylethyl 7-(4-amino-3-nitrophenyl)-2,3-dihydro-1,4-benzoxazepine-4(5H)-carboxylate (Example 26, 1.35 g, 3.1 mmol) in ethyl acetate (30 mL) was added 5% palladium on carbon (wet). The resulting suspension was subjected to an atmosphere of hydrogen (40 psi) for 15 h. The catalyst was then removed by filtration through celite. The filtrate was concentrated to provide 1,1-dimethylethyl 7-(3,4-diaminophenyl)-2,3-dihydro-1,4-benzoxazepine-4(5H)-carboxylate (748 mg, 210 mmol, 68% yield) as an orange viscous syrup. MS (EI) for C₂₀H₂₅N₃O₃: 356 (MH⁺).

STEP 2: A solution of 1,1-dimethylethyl 7-(3,4-diaminophenyl)-2,3-dihydro-1,4-benzoxazepine-4(5H)-carboxylate (750 mg, 2.10 mmol) in ethyl acetate (20 mL) was treated with ethyl isothiocyanate (184 uL, 2.10 mmol). The mixture was heated to 60° C. for 4.25 h. After cooling to rt, water was added and the layers were partitioned. The organic phase was washed once with saturated sodium bicarbonate, dried over magnesium sulfate, filtered, and concentrated. The residue was then dissolved in ethyl acetate (20 mL), and N-(3-dimethylaminopropyl)-N′-ethylcarbodiimide hydrochloride (403 mg, 2.10 mmol) was added. The mixture was heated to 60° C. for 50 minutes before cooling to rt. Water was added, and the biphasic mixture was partitioned. The aqueous phase was extracted with ethyl acetate. The combined organic extracts were dried over magnesium sulfate, filtered, and concentrated. The residue was purified by silica gel chromatography (gradient: 98:2 dichloromethane:methanol to 90:10 dichloromethane:methanol) to provide 1,1-dimethylethyl 7-[2-(ethylamino)-1H-benzimidazol-6-yl]-2,3-dihydro-1,4-benzoxazepine-4(5H)-carboxylate (337 mg, 0.83 mmol, 39% yield) as an orange film. ¹H NMR (400 MHz, DMSO-d6) δ 10.83 (br s, 1H), 7.47-7.38 (m, 2H), 7.32 (s, 1H), 7.20-7.08 (m, 2H), 7.05-6.96 (m, 1H), 6.68-6.58 (m, 1H), 4.55-4.40 (m, 2H), 4.09-3.98 (m, 2H), 3.77-3.65 (m, 2H), 3.33-3.26 (m, 2H), 1.41-1.28 (m, 9H), 1.18 (t, 3H); MS (EI) for C₂₃H₂₈N₄O₃: 409 (MH⁺).

STEP 3: A solution of hydrogen chloride in dioxane (4 M, 2.09 mL, 8.3 mmol) was added to 1,1-dimethylethyl 7-[2-(ethylamino)-1H-benzimidazol-6-yl]-2,3-dihydro-1,4-benzoxazepine-4(5H)-carboxylate (337 mg, 0.83 mmol) in methanol (4 mL). The mixture was heated to 60° C. and stirred for 1 h before cooling to rt. The volatile materials were removed to provide N-ethyl-5-(2,3,4,5-tetrahydro-1,4-benzoxazepin-7-yl)-1H-benzimidazol-2-amine dihydrochloride in quantitative yield. ¹H NMR (400 MHz, DMSO-d6) δ 12.85 (d, 2H), 9.48 (br s, 2H), 9.11-9.03 (m, 1H), 7.77 (d, 1H), 7.63 (dd, 1H), 7.55 (d, 1H), 7.52-7.42 (m, 2H), 7.19 (d, 1H), 4.42 (br s, 2H), 4.28-4.20 (m, 2H), 3.55-3.41 (m, 4H), 1.26 (t, 3H); MS (EI) for C₁₈H₂₀N₄O: 309 (MH⁺).

STEP 4: To a mixture of N-ethyl-5-(2,3,4,5-tetrahydro-1,4-benzoxazepin-7-yl)-1H-benzimidazol-2-amine dihydrochloride (50 mg, 0.13 mmol) and 4-chloro-7-methoxy-2-methylquinazoline (27 mg, 0.13 mmol) in NMP (1 mL) was added diisopropylethylamine (91 uL, 0.52 mmol). The mixture was heated to 90° C. and stirred for 1 h. After cooling to rt, water was added, and the resulting aqueous mixture was extracted twice with 10% methanol in ethyl acetate. The organic extracts were combined, dried over magnesium sulfate, filtered, and then concentrated. The residue was purified by reverse-phase preparative HPLC to provide N-ethyl-6-{4-[2-methyl-7-(methyloxy)quinazolin-4-yl]-2,3,4,5-tetrahydro-1,4-benzoxazepin-7-yl}-1H-benzimidazol-2-amine as an acetate salt (34.2 mg, 0.063 mmol, 49% yield). ¹H NMR (400 MHz, DMSO-d6) δ 11.91 (br s, 1H), 10.83 (br s, 1H), 7.92 (d, 1H), 7.58 (d, 1H), 7.43 (dd, 1H), 7.35 (s, 1H), 7.20-7.10 (m, 3H), 7.05-6.97 (m, 2H), 6.61 (br s, 1H), 4.99 (s, 2H), 4.44-4.38 (m, 2H), 4.18-4.11 (m, 2H), 3.33-3.25 (m, 2H), 2.44 (s, 3H), 1.91 (s, 4H), 1.19 (t, 3H); MS (EI) for C₂₈H₂₈N₆O₂: 481 (MH⁺).

Using analogous synthetic techniques and substituting with alternative starting reagents in step 4 the following compounds of the invention were prepared. Protecting group introduction and removal steps were conducted as required according to literature techniques appropriate for a given protecting group (see for example: Greene and Wuts, Protective Groups in Organic Synthetic, Wiley-Interscience). Alternative starting materials were obtained commercially unless otherwise indicated.

N-ethyl-6-{4-[2-methyl-6,7-bis(methyloxy)quinazolin-4-yl]-2,3,4,5-tetrahydro-1,4-benzoxazepin-7-yl}-1H-benzimidazol-2-amine. Prepared as an acetate salt according to the method of example 11 by using 4-chloro-6,7-dimethoxy-2-methylquinazoline in step 4. ¹H NMR (400 MHz, DMSO-d6) δ 10.80 (s, 1H), 7.68 (s, 1H), 7.46 (d, 1H), 7.35 (br s, 1H), 7.21-7.06 (m, 3H), 7.06-6.97 (m, 2H), 6.60 (t, 1H), 4.94 (s, 2H), 4.49-4.41 (m, 2H), 4.09-3.97 (m, 2H), 3.87 (s, 3H), 3.51 (s, 3H), 3.32-3.26 (m, 2H), 2.47 (s, 3H), 1.90 (s, 3H), 1.18 (t, 3H); MS (EI) for C₂₉H₃₀N₆O₃: 511 (MH⁺).

N-ethyl-6-[4-(7-fluoroquinazolin-4-yl)-2,3,4,5-tetrahydro-1,4-benzoxazepin-7-yl]-1H-benzimidazol-2-amine. Prepared as an acetate salt according to the method of example 11 by using 4-chloro-7-fluoroquinazoline in step 4. ¹H NMR (400 MHz, DMSO-d6) δ 10.79 (s, 1H), 8.52 (s, 1H), 8.15 (dd, 1H), 7.65-7.58 (m, 1H), 7.56-7.49 (m, 1H), 7.48-7.32 (m, 3H), 7.23-7.08 (m, 2H), 6.97 (d, 1H), 6.60 (t, 1H), 5.11 (s, 2H), 4.54-4.44 (m, 2H), 4.27-4.16 (m, 2H), 3.31-3.24 (m, 2H), 1.90 (s, 2H), 1.18 (t, 3H); MS (EI) for C₂₆H₂₃FN₆O: 455 (MH⁺).

4-{7-[2-(ethylamino)-1H-benzimidazol-6-yl]-2,3-dihydro-1,4-benzoxazepin-4(5H)-yl}-2-methylquinazolin-7-ol. Prepared as an acetate salt according to the method of example 11 by using 7-(benzyloxy)-4-chloro-2-methylquinazoline in step 4 followed by benzyl deprotection. ¹H NMR (400 MHz, DMSO-d6) δ 7.87 (d, 1H), 7.57 (d, 1H), 7.43 (dd, 1H), 7.33 (s, 1H), 7.19-7.11 (m, 2H), 7.02-6.93 (m, 2H), 6.91 (d, 1H), 6.83-6.73 (m, 1H), 4.94 (s, 2H), 4.43-4.35 (m, 2H), 4.15-4.08 (m, 2H), 3.36-3.28 (m, 2H), 2.41 (s, 3H), 1.87 (s, 10H), 1.18 (t, 3H); MS (EI) for C₂₇H₂₆N₆O₂: 467 (MH⁺).

N-ethyl-6-[4-(2-methylquinolin-4-yl)-2,3,4,5-tetrahydro-1,4-benzoxazepin-7-yl]-1H-benzimidazol-2-amine. Prepared as an acetate salt according to the method of example 11 by using 4-chloro-2-methylquinoline in step 4. ¹H NMR (400 MHz, DMSO-d6) δ 10.84 (br s, 1H), 7.95 (d, 1H), 7.85 (d, 1H), 7.64 (t, 1H), 7.61-7.56 (m, 1H), 7.50 (dd, 1H), 7.43 (t, 1H), 7.38 (s, 1H), 7.17 (br s, 2H), 7.08 (d, 1H), 6.98 (s, 1H), 6.63 (t, 1H), 4.55 (s, 2H), 4.39-4.30 (m, 2H), 3.79-3.70 (m, 2H), 3.35-3.27 (m, 2H), 2.55 (s, 3H), 1.18 (t, 3H); MS (EI) for C₂₈H₂₇N₅O: 450 (MH⁺).

N-ethyl-6-{4-[2-methyl-7-(methyloxy)quinolin-4-yl]-2,3,4,5-tetrahydro-1,4-benzoxazepin-7-yl}-1H-benzimidazol-2-amine. Prepared as an acetate salt according to the method of example 11 by using 4-chloro-7-methoxy-2-methylquinoline in step 4. ¹H NMR (400 MHz, DMSO-d6) δ 10.81 (br s, 1H), 7.84 (d, 1H), 7.57 (br s, 1H), 7.49 (dd, 1H), 7.37 (br s, 1H), 7.26 (d, 1H), 7.17 (br s, 2H), 7.09-7.03 (m, 2H), 6.84 (s, 1H), 6.61 (t, 1H), 4.53 (s, 2H), 4.37-4.30 (m, 2H), 3.88 (s, 3H), 3.75-3.67 (m, 2H), 3.34-3.27 (m, 2H), 2.51 (s, 3H), 1.93-1.89 (m, 3H), 1.18 (t, 3H); MS (EI) for C₂₉H₂₉N₅O₂: 480 (MH⁺).

6-{4-[6,7-bis(methyloxy)quinazolin-4-yl]-2,3,4,5-tetrahydro-1,4-benzoxazepin-7-yl}-N-ethyl-1H-benzimidazol-2-amine. Prepared as an acetate salt according to the method of example 11 by using 4-chloro-6,7-dimethoxyquinazoline in step 4. ¹H NMR (400 MHz, DMSO-D6-d6) δ 8.48 (s, 1H), 7.68 (s, 1H), 7.46-7.41 (m, 1H), 7.36-7.32 (m, 1H), 7.20 (s, 1H), 7.17-7.13 (m, 2H), 7.07 (s, 1H), 7.01 (d, 1H), 6.60 (d, 1H), 4.99 (s, 2H), 4.53-4.48 (m, 2H), 4.07-4.02 (m, 2H), 3.90 (s, 3H), 3.53 (s, 3H), 3.33-3.28 (m, 2H), 1.92-1.89 (m, 4H), 1.18 (t, 3H); MS (EI) for C₂₈H₂₈N₆O₃: 497 (MH⁺).

N-ethyl-6-[4-(2-ethylquinazolin-4-yl)-2,3,4,5-tetrahydro-1,4-benzoxazepin-7-yl]-1H-benzimidazol-2-amine. Prepared as an acetate salt according to the method of example 11 by using 4-chloro-2-ethylquinazoline in step 4. ¹H NMR (400 MHz, DMSO-D6-d6) δ 8.03 (d, 1H), 7.78-7.69 (m, 2H), 7.60 (d, 1H), 7.47-7.39 (m, 2H), 7.35 (s, 1H), 7.19-7.11 (m, 2H), 6.96 (d, 1H), 6.69 (s, 1H), 5.05 (s, 2H), 4.47-4.40 (m, 2H), 4.24-4.18 (m, 2H), 3.37-3.28 (m, 2H), 2.71 (q, 2H), 1.90 (s, 4H), 1.22-1.15 (m, 6H); MS (EI) for C₂₈H₂₈N₆O: 465 (MH⁺).

6-{4-[6,7-bis(methyloxy)quinolin-4-yl]-2,3,4,5-tetrahydro-1,4-benzoxazepin-7-yl}-N-ethyl-1H-benzimidazol-2-amine. Prepared as an acetate salt according to the method of example 11 by using 4-chloro-6,7-dimethoxyquinoline in step 4. ¹H NMR (400 MHz, DMSO-d6) δ 10.79 (br s, 1H), 8.47 (d, 1H), 7.67 (br s, 1H), 7.52-7.46 (m, 1H), 7.42-7.32 (m, 1H), 7.31 (s, 1H), 7.21-7.13 (m, 2H), 7.11 (s, 1H), 7.06 (d, 1H), 6.95 (d, 1H), 6.60 (t, 1H), 4.55 (s, 2H), 4.42-4.35 (m, 2H), 3.89 (s, 3H), 3.75-3.67 (m, 2H), 3.53 (s, 3H), 3.32-3.26 (m, 2H), 1.91 (s, 3H), 1.18 (t, 3H); MS (EI) for C₂₉H₂₉N₅O₃: 496 (MH⁺).

N-ethyl-6-{4-[6-(methyloxy)quinolin-4-yl]-2,3,4,5-tetrahydro-1,4-benzoxazepin-7-yl}-1H-benzimidazol-2-amine. Prepared as an acetate salt according to the method of example 11 by using 4-chloro-6-methoxyquinoline in step 4. ¹H NMR (400 MHz, DMSO-d6) δ 10.81 (br s, 1H), 8.53 (d, 1H), 7.86 (d, 1H), 7.67 (br s, 1H), 7.49 (d, 1H), 7.45-7.29 (m, 2H), 7.24-7.11 (m, 3H), 7.10-7.02 (m, 2H), 6.61 (t, 1H), 4.57 (s, 2H), 4.43-4.35 (m, 2H), 3.78-3.69 (m, 2H), 3.58 (s, 3H), 3.33-3.27 (m, 2H), 1.90 (s, 3H), 1.18 (t, 3H); MS (EI) for C₂₈H₂₇N₅O₂: 466 (MH⁺).

N-ethyl-6-{4-[2-ethyl-7-(methyloxy)quinazolin-4-yl]-2,3,4,5-tetrahydro-1,4-benzoxazepin-7-yl}-1H-benzimidazol-2-amine. Prepared as its acetate salt according to the method of example 11 by using 4-chloro-2-ethyl-7-methoxyquinazoline (prepared according to the methods described by Abe et al. J. Med. Chem. 1998, 41, 4062-4079 using 2-amino-4-methoxybenzoic acid and propionyl chloride) in step 4. ¹H NMR (400 MHz, DMSO-D6-d6) δ 7.93 (d, 1H), 7.58 (d, 1H), 7.44-7.38 (m, 1H), 7.34 (s, 1H), 7.19-7.10 (m, 3H), 7.04-6.99 (m, 1H), 6.94 (d, 1H), 6.64 (s, 1H), 5.02 (s, 2H), 4.43 (s, 2H), 4.17 (s, 2H), 3.88 (s, 3H), 3.33-3.28 (m, 2H), 2.68 (q, 2H), 1.89 (s, 6H), 1.21-1.16 (m, 6H); MS (EI) for C₂₉H₃₀N₆O₂.2C₂H₄O₂: 495 (MH⁺).

N-ethyl-6-{4-[7-(methyloxy)quinolin-4-yl]-2,3,4,5-tetrahydro-1,4-benzoxazepin-7-yl}-1H-benzimidazol-2-amine. Prepared as an acetate salt according to the method of example 11 by using 4-chloro-7-methoxyquinoline in step 4. ¹H NMR (400 MHz, DMSO-d6) δ 10.81 (br s, 1H), 8.53 (d, 1H), 7.91 (d, 1H), 7.60 (s, 1H), 7.49 (dd, 1H), 7.38 (br s, 1H), 7.31 (d, 1H), 7.24-7.09 (m, 3H), 7.05 (d, 1H), 6.88 (d, 1H), 6.67-6.57 (m, 1H), 4.60 (s, 2H), 4.39-4.30 (m, 2H), 3.82-3.74 (m, 2H), 3.33-3.27 (m, 2H), 1.90 (s, 3H), 1.18 (t, 3H); MS (EI) for C₂₈H₂₇N₅O₂: 466 (MH⁺).

N-ethyl-6-[4-(7-fluoro-2-methylquinazolin-4-yl)-2,3,4,5-tetrahydro-1,4-benzoxazepin-7-yl]-H-benzimidazol-2-amine. Prepared as an acetate salt according to the method of example 11 by using 4-chloro-7-fluoro-2-methylquinazoline (prepared according to the methods described by Abe et al. J. Med. Chem. 1998, 41, 4062-4079 using 2-amino-4-fluorobenzoic acid and acetyl chloride) in step 4. ¹H NMR (400 MHz, DMSO-d6) δ 8.13-8.06 (m, 1H), 7.61 (d, 1H), 7.45-7.41 (m, 2H), 7.37-7.30 (m, 2H), 7.19-7.12 (m, 2H), 6.98 (d, 1H), 6.68-6.62 (m, 1H), 5.05 (s, 2H), 4.43 (d, 2H), 4.20 (d, 2H), 3.35-3.28 (m, 2H), 2.46 (s, 3H), 1.89 (s, 4H), 1.18 (t, 3H); MS (EI) for C₂₇H₂₅FN₆O: 469 (MH⁺).

N-ethyl-6-[4-(7-fluoro-2-methylquinolin-4-yl)-2,3,4,5-tetrahydro-1,4-benzoxazepin-7-yl]-1H-benzimidazol-2-amine. Prepared as an acetate salt according to the method of example 11 by using 4-chloro-7-fluoro-2-methylquinoline in step 4. ¹H NMR (400 MHz, DMSO-d6) δ 10.86 (br s, 1H), 8.00 (dd, 1H), 7.64-7.53 (m, 2H), 7.49 (dd, 1H), 7.43-7.30 (m, 2H), 7.17 (br s, 2H), 7.07 (d, 1H), 6.96 (s, 1H), 6.63 (t, 1H), 4.57 (s, 2H), 4.39-4.30 (m, 2H), 3.80-3.69 (m, 2H), 3.36-3.26 (m, 2H), 2.54 (s, 3H), 1.89 (s, 3H), 1.18 (t, 3H); MS (EI) for C₂₈H₂₆FN₅O: 468 (MH⁺).

N-ethyl-6-(4-pyrimidin-4-yl-2,3,4,5-tetrahydro-1,4-benzoxazepin-7-yl)-1H-imidazo[4,5-b]pyridin-2-amine. Prepared as an acetate salt according to the method of example 11 by using tert-butyl 7-(6-amino-5-nitropyridin-3-yl)-2,3-dihydro-1,4-benzoxazepine-4(5H)-carboxylate (Example 26) in step 1 and 4-chloropyrimidine in step 4. ¹H NMR (400 MHz, DMSO-D6-d6) δ 8.13-8.06 (m, 1H), 7.61 (d, 1H), 7.45-7.41 (m, 2H), 7.37-7.30 (m, 2H), 7.19-7.12 (m, 2H), 6.98 (d, 1H), 6.68-6.62 (m, 1H), 5.05 (s, 2H), 4.43 (d, 2H), 4.20 (d, 2H), 3.35-3.28 (m, 2H), 2.46 (s, 3H), 1.89 (s, 4H), 1.18 (t, 3H); MS (EI) for C₂₁H₂₁N₇O: 388 (MH⁺).

N-ethyl-6-{4-[2-methyl-7-(methyloxy)quinazolin-4-yl]-2,3,4,5-tetrahydro-1,4-benzoxazepin-7-yl}-1H-imidazo[4,5-b]pyridin-2-amine. Prepared according to the method of example 11 by using tert-butyl 7-(6-amino-5-nitropyridin-3-yl)-2,3-dihydro-1,4-benzoxazepine-4(5H)-carboxylate (Example 26) in step 1 and 4-chloro-7-methoxy-2-methylquinazoline in step 4. ¹H NMR (400 MHz, DMSO-d6) δ 8.22 (s, 0.5H), 8.07 (s, 0.5H), 7.90 (d, 1H), 7.68-7.42 (m, 3H), 7.17-6.84 (m, 4H), 5.00 (s, 2H), 4.47-4.39 (m, 2H), 4.18-4.10 (m, 2H), 3.88 (s, 3H), 3.40-3.30 (m, 2H), 2.43 (s, 3H), 1.26-1.13 (m, 3H); MS (EI) for C₂₇H₂₇N₇O₂: 482 (MH⁺).

6-{4-[2,5-dimethyl-6-(phenylamino)pyrimidin-4-yl]-2,3,4,5-tetrahydro-1,4-benzoxazepin-7-yl}-N-ethyl-1H-benzimidazol-2-amine. Prepared as a trifluoroacetate salt according to the method of example 11 by using 6-chloro-2,5-dimethyl-N-phenylpyrimidin-4-amine (prepared according to the methods described by Chen et al. J. Med. Chem. 1996, 39, 4358-4360 using 4,6-dichloro-2,5-dimethylpyrimidine and aniline) in step 4. ¹H NMR (400 MHz, DMSO-d6) δ 8.99 (t, 1H), 7.60 (d, 1H), 7.56-7.39 (m, 5H), 7.29 (s, 2H), 7.11-6.97 (m, 2H), 4.65 (s, 2H), 4.35-4.29 (m, 2H), 3.87-3.80 (m, 2H), 3.47-3.37 (m, 2H), 2.30 (s, 3H), 2.09 (s, 3H), 1.26 (t, 3H); MS (EI) for C₃₀H₃₁N₇O: 506 (MH⁺).

N-ethyl-6-{4-[6-(phenylamino)pyrimidin-4-yl]-2,3,4,5-tetrahydro-1,4-benzoxazepin-7-yl}-1H-benzimidazol-2-amine. Prepared according to the method of example 11 by using 6-chloro-N-phenylpyrimidin-4-amine (reagent preparation 49) in step 4. ¹H NMR (400 MHz, DMSO-d6) δ 12.03 (br s, 1H), 9.01 (s, 1H), 8.13 (s, 1H), 7.50 (m, 6H), 7.01 (m, 6H), 6.02 (s, 1H), 4.87 (br s, 2H), 4.24 (br s, 4H), 3.48 (q, 2H), 1.23 (t, 3H); MS (EI) for C₂₈H₂₇N₇O: 478.3 (MH⁺).

N-ethyl-6-[4-(6-{[4-(methyloxy)phenyl]amino}pyrimidin-4-yl)-2,3,4,5-tetrahydro-1,4-benzoxazepin-7-yl]-1H-benzimidazol-2-amine. Prepared according to the method of example 11 by using 6-chloro-N-(4-methoxyphenyl)pyrimidin-4-amine (reagent preparation 49) in step 4. ¹H NMR (400 MHz, DMSO-d6) δ 12.03 (br s, 1H), 9.23 (br s, 1H), 9.02 (t, 1H), 8.13 (s, 1H), 7.50 (m, 5H), 7.30 (d, 2H), 7.01 (d, 1H), 6.75 (br s, 2H), 6.02 (s, 1H), 4.87 (br s, 2H), 4.24 (br s, 4H), 3.77 (s, 3H), 3.48 (q, 2H), 1.23 (t, 3H); MS (EI) for C₂₉H₂₉N₇O₂: 508.2 (MH⁺).

N-ethyl-6-(4-pyrimidin-4-yl-2,3,4,5-tetrahydro-1,4-benzoxazepin-7-yl)-1H-benzimidazol-2-amine. Prepared according to the method of example 11 by using 4-chloropyrimidine in step 4. ¹H NMR (400 MHz, DMSO-d6) δ 12.03 (br s, 1H), 9.23 (br s, 1H), 8.75 (s, 1H), 8.47 (d, 1H), 8.03 (br s, 1H), 7.50 (m, 5H), 7.01 (d, 1H), 5.02 (br s, 2H), 4.34 (br s, 4H), 3.48 (q, 2H), 1.23 (t, 3H); MS (EI) for C₂₂H₂₂N₆O: 387.3 (MH⁺).

N-ethyl-6-[4-(6-{[3-(methyloxy)phenyl]amino}pyrimidin-4-yl)-2,3,4,5-tetrahydro-1,4-benzoxazepin-7-yl]-1H-benzimidazol-2-amine. Prepared according to the method of example 11 by using 6-chloro-N-(3-methoxyphenyl)pyrimidin-4-amine (reagent preparation 49) in step 4. ¹H NMR (400 MHz, DMSO-d6) δ 12.03 (br s, 1H), 8.20 (s, 1H), 7.60 (s, 1H), 7.40 (m, 2H), 7.15 (m, 7H), 6.70 (br s, 1H), 6.50 (m, 1H), 6.15 (s, 1H), 4.72 (br s, 2H), 4.18 (s, 4H), 3.77 (s, 3H), 3.48 (q, 2H), 1.23 (t, 3H); MS (EI) for C₂₉H₂₉N₇O₂: 508.2 (MH⁺).

N-ethyl-6-[4-(5-methyl-6-{[4-(methyloxy)phenyl]amino}pyrimidin-4-yl)-2,3,4,5-tetrahydro-1,4-benzoxazepin-7-yl]-1H-benzimidazol-2-amine. Prepared according to the method of example 11 by using 6-chloro-N-(4-methoxyphenyl)-5-methylpyrimidin-4-amine (reagent preparation 49) in step 4. ¹H NMR (400 MHz, DMSO-d6) δ 12.03 (br s, 1H), 8.20 (s, 1H), 8.01 (s, 1H), 7.51 (m, 5H), 7.18 (m, 2H), 7.01 (d, 1H), 6.81 (d, 2H), 6.75 (br s, 1H), 4.50 (s, 2H), 4.23 (br s, 2H), 3.78 (br s, 2H), 3.33 (q, 2H), 2.18 (s, 3H), 1.81 (s, 3H), 1.23 (t, 3H); MS (EI) for C₃₀H₃₁N₇O₂: 522.0 (MH⁺).

N-ethyl-6-{4-[5-methyl-6-(phenylamino)pyrimidin-4-yl]-2,3,4,5-tetrahydro-1,4-benzoxazepin-7-yl}-1H-benzimidazol-2-amine. Prepared according to the method of example 11 by using 6-chloro-5-methyl-N-phenylpyrimidin-4-amine (reagent preparation 49) in step 4. ¹H NMR (400 MHz, MeOH-d4) δ 8.01 (s, 1H), 7.51 (m, 10H), 7.02 (d, 1H), 6.75 (brs, 2H), 4.50 (brs, 4H), 4.14 (brs, 4H), 3.48 (q, 2H), 2.21 (s, 3H), 1.38 (t, 3H); MS (EI) for C₂₉H₂₉N₇O: 492.2 (MH⁺).

### Example 12

### 4-(4-{5-[(4-fluorophenyl)methyl]-6-methylpyrimidin-4-yl}-2,3,4,5-tetrahydro-1,4-benzoxazepin-7-yl)-N-methylbenzamide

STEP 1: A suspension of {4-[(methyloxy)carbonyl]phenyl}boronic acid (0.36 g, 2.0 mmol), 1,1-dimethylethyl 7-bromo-2,3-dihydro-1,4-benzoxazepine-4(5H)-carboxylate (0.66 g, 2.0 mmol), [1,1′-bis(diphenylphosphino)ferrocene]dichloropalladium(II) (70.0 mg, 0.10 mmol), and tripotassium phosphate (1.30 g, 12.0 mmol) in dioxane (20 mL) was refluxed for 3 h, and then cooled to room temperature. The reaction mixture was partitioned between ethyl acetate (50 mL) and water (80 mL), the organic layer was washed with brine (40 mL), dried over sodium sulfate then filtered and concentrated. Column chromatography on silica (ethyl acetate:hexanes 1:4) gave 1,1-dimethylethyl 7-{4-[(methyloxy)carbonyl]phenyl}-2,3-dihydro-1,4-benzoxazepine-4(5H)-carboxylate (0.47 g, 60% yield). ¹H NMR (400 MHZ, DMSO-D₆); 8.11 (m, 2H), 7.63-7.52 (m, 2H), 7.43 (m, 2H), 7.10 (t, 1H), 4.57-4.43 (br, 2H), 4.08 (m, 2H), 3.82 (m, 2H), 1.40 (s, 9H); MS (EI) for C₂₂H₂₅NO₅: 469 (MH⁺).

STEP 2: To a solution of 1,1-dimethylethyl 7-{4-[(methyloxy)carbonyl]phenyl}-2,3-dihydro-1,4-benzoxazepine-4(5H)-carboxylate (1.90 g, 4.96 mmol) in dry methanol (10 mL) was added drop wise 4 N hydrogen chloride in dioxane (10 mL) at room temperature. The reaction mixture was warmed to 55° C. for 60 min, at which time it was cooled to room temperature. The precipitated product was isolated by filtration, washed with diethyl ether, and dried to yield methyl 4-(2,3,4,5-tetrahydro-1,4-benzoxazepin-7-yl)benzoate hydrochloride (1.53 g, 97% yield) as a white solid.

STEP 3: A suspension of -(2,3,4,5-tetrahydro-1,4-benzoxazepin-7-yl)benzoate hydrochloride (1.30 g, 4.14 mmol), 4-chloro-5-[(4-fluorophenyl)methyl]-6-methylpyrimidine (reagent preparation 5) (0.98 g, 4.14 mmol), and potassium carbonate (1.71 g, 12.4 mmol) in DMF (20 mL) was heated to 130° C. for 18 h. The reaction mixture was cooled to room temperature, diluted with ethyl acetate (40 mL), and then washed with water (50 mL) and brine (20 mL). The organic layer was dried over sodium sulfate then filtered and concentrated. Column chromatography on silica (gradient 10 to 20% ethyl acetate in hexane) followed by recrystallization from 1:1 ethyl acetate and ether (40 mL) provided methyl 4-(4-{5-[(4-fluorophenyl)methyl]-6-methylpyrimidin-4-yl}-2,3,4,5-tetrahydro-1,4-benzoxazepin-7-yl)benzoate (1.05 g, 50% yield) as a white solid. ¹H NMR (400 MHZ, DMSO-D₆): 8.48 (s, 1H), 8.01 (d, 2H), 7.62 (d, 2H), 7.56 (dd, 1H), 7.11 (d, 4H), 7.02 (d, 1H), 6.91 (d, 1H), 4.52 (s, 2H), 4.32 (m, 2H), 3.95 (s, 2H), 3.90 (s, 3H), 3.76 (m, 2H), 2.13 (s, 3H); MS (EI) for C₂₉H₂₆FN₃O₃: 484 (MH⁺).

STEP 4: To a solution of methyl 4-(4-{5-[(4-fluorophenyl)methyl]-6-methylpyrimidin-4-yl}-2,3,4,5-tetrahydro-1,4-benzoxazepin-7-yl)benzoate (1.0 g, 2.0 mmol) in 1:1 methanol and THF (10 mL) was added drop wise 2N aqueous potassium hydroxide (8 mL). The reaction mixture was stirred at room temperature for 18 h and then refluxed for 90 min. The mixture was cooled by adding ice, and the pH adjusted to 6 with 2N aqueous hydrochloric acid. The precipitate was filtered, washed with water, azeotroped with toluene (20 mL), and dried to afford 4-(4-{5-[(4-fluorophenyl)methyl]-6-methylpyrimidin-4-yl}-2,3,4,5-tetrahydro-1,4-benzoxazepin-7-yl)benzoic acid (0.97 g, 100% yield).

STEP 5: To a solution of 4-(4-{5-[(4-fluorophenyl)methyl]-6-methylpyrimidin-4-yl}-2,3,4,5-tetrahydro-1,4-benzoxazepin-7-yl)benzoic acid (0.50 g, 1.07 mmol) and DMF (20 μL) in chloroform (15 mL) was added drop wise oxalyl chloride (0.35 mL, 4.0 mmol). The reaction mixture was refluxed for 15 min, and then concentrated to give 4-(4-{5-[(4-fluorophenyl)methyl]-6-methylpyrimidin-4-yl}-2,3,4,5-tetrahydro-1,4-benzoxazepin-7-yl)benzoyl chloride as an oil.

STEP 6: To a solution of 4-(4-{5-[(4-fluorophenyl)methyl]-6-methylpyrimidin-4-yl}-2,3,4,5-tetrahydro-1,4-benzoxazepin-7-yl)benzoyl chloride (30.0 mg, 62 μmol) in THF (5 mL) was added drop wise 40% aqueous methylamine (0.25 mL, 2.0 mmol) at 0° C. The reaction mixture was stirred at room temperature for 1 h, at which time it was concentrated. The resulting solid was dissolved in chloroform (30 mL), washed with water (20 mL), and dried over sodium sulfate then filtered. To the solution was then added drop wise 4N hydrogen chloride in dioxane (0.25 mL) and the mixture concentrated. The residue was taken up in 4:1 water and acetonitrile (2 mL) and lyophilized to give 4-(4-{5-[(4-fluorophenyl)methyl]-6-methylpyrimidin-4-yl}-2,3,4,5-tetrahydro-1,4-benzoxazepin-7-yl)-N-methylbenzamide hydrochloride salt (16.5 mg, 53% yield) as a white powder. ¹H NMR (400 MHZ, DMSO-d₆): 8.79 (s, 1H), 8.53 (dd, 1H), 7.97 (d, 2H), 7.65 (d, 2H), 7.57 (d, 1H), 7.30-7.13 (m, 5H), 6.97 (d, 1H), 4.92 (s, 1H), 4.35 (br 2H), 4.04-3.97 (m, 4H), 3.83 (d, 3H), 2.24 (s, 3H); MS (EI) for C₂₉H₂₇FN₄O₂: 483 (MH⁺).

Using analogous synthetic techniques and substituting with alternative starting reagents in step 6 the following compounds of the invention were prepared. Alternative starting materials were obtained commercially unless otherwise indicated.

N-cyclopropyl-4-(4-{5-[(4-fluorophenyl)methyl]-6-methylpyrimidin-4-yl}-2,3,4,5-tetrahydro-1,4-benzoxazepin-7-yl)benzamide. Synthesized according to the method of example 12 using cyclopropylamine in step 6. ¹H NMR (400 MHz, d₆-DMSO): 8.79 (s, 1H), 8.53 (d, 1H), 7.92 (d, 2H), 7.61 (d, 2H), 7.58 (d, 1H), 7.12 (m, 5H), 7.02 (d, 1H), 4.92 (s, 2H), 4.38 (br s, 2H), 4.05 (s, 2H), 4.00 (br s, 2H), 2.88 (m, 1H), 2.25 (s, 3H), 0.73 (m, 2H), 0.61 (m, 2H); MS (EI) for C₃₁H₂₆FN₄O₂: 509 (MH⁺).

4-(4-{5-[(4-fluorophenyl)methyl]-6-methylpyrimidin-4-yl}-2,3,4,5-tetrahydro-1,4-benzoxazepin-7-yl)-N-[(3S)-pyrrolidin-3-yl]benzamide. Synthesized according to the method of example 12 using (3S)—N^{l}—BOC-pyrrolidin-3-ylamine in step 6. ¹H NMR (400 MHz, d₆-DMSO): 9.38 (br s, 1H), 9.14 (br s, 1H), 8.03 (d, 2H), 7.67 (d, 2H), 7.59 (d, 1H), 7.21 (m, 4H), 7.05 (d, 1H), 4.92 (s, 2H), 4.60 (m, 1H), 4.36 (br s, 2H), 4.08 (br s, 2H), 3.98 (br s, 2H), 3.68 (m, 3H), 3.49 (m, 3H), 3.24 (m, 1H), 2.24 (s, 3H); MS (EI) for C₃₂H₃₂FN₅O₂: 538 (MH⁺).

N-(2,2-difluoroethyl)-4-(4-{5-[(4-fluorophenyl)methyl]-6-methylpyrimidin-4-yl}-2,3,4,5-tetrahydro-1,4-benzoxazepin-7-yl)benzamide. Synthesized according to the method of example 12 using 2,2-difluoroethylamine in step 6. ¹H NMR (400 MHz, d₆-DMSO); 8.95 (t, 1H), 8.80 (s, 1H), 7.98 (d, 2H), 7.68 (d, 2H), 7.59 (d, 1H), 7.21 (m, 4H), 7.04 (d, 1H), 6.17 (tt, 1H), 4.93 (s, 2H), 4.37 (br s, 2H), 4.08 (s, 2H), 3.99 (br s, 2H), 3.61 (br m, 2H), 2.25 (s, 3H); MS (EI) for C₃₀H₂₇F₃N₄O₂: 533 (MH⁺).

4-(4-{5-[(4-fluorophenyl)methyl]-6-methylpyrimidin-4-yl}-2,3,4,5-tetrahydro-1,4-benzoxazepin-7-yl)benzamide. Synthesized according to the method of example 12 using ammonia in step 6. ¹H NMR (400 MHz, d₆-DMSO): 8.63 (br s, 1H), 8.05 (br s, 1H), 7.96 (d, 2H), 7.59 (d, 2H), 7.55 (dd, 1H), 7.41 (br s, 1H), 7.16 (m, 4H), 7.10 (br s, 1H), 7.01 (d, 1H), 4.70 (br s, 2H), 4.32 (m, 2H), 4.00 (s, 2H), 3.86 (m, 2H), 2.20 (s, 3H). MS (EI) for C₂₈H₂₅FN₄O₂: 469 (MH⁺).

4-(4-{2-[(dimethylamino)methyl]-6,6-dimethyl-5,6,7,8-tetrahydroquinazolin-4-yl}-2,3,4,5-tetrahydro-1,4-benzoxazepin-7-yl)-N-methylbenzamide. Prepared according to the method of example 12 by using 1-(4-chloro-6,6-dimethyl-5,6,7,8-tetrahydroquinazolin-2-yl)-N,N-dimethylmethanamine (reagent preparation 17) in step 3. ¹H NMR (400 MHz, methanol-d₄): 7.88 (d, 2H), 7.72 (d, 2H), 7.66 (d, 1H), 7.52 (dd, 1H), 7.04 (d, 1H), 4.78 (s, 2H), 4.37 (m, 2H), 4.01 (m, 2H), 3.76 (s, 2H), 2.94 (s, 3H), 2.80 (t, 2H), 2.50 (s, 2H), 2.47 (s, 6H), 1.92 (s, 3H), 1.68 (t, 2H), 0.90 (s, 6H); MS (EI) for C₃₀H₃₇N₅O₂: 500 (MH⁺).

N-methyl-4-(4-quinolin-4-yl-2,3,4,5-tetrahydro-1,4-benzoxazepin-7-yl)benzamide. Prepared according to the method of example 12 using 4-chloroquinoline in step 3 and methylamine in step 6. ¹H NMR (400 MHz, DMSO-d6); δ 8.60 (d, 1H), 8.53-8.46 (m, 1H), 8.06 (d, 1H), 7.98-7.70 (m, 6H), 7.68-7.62 (m, 1H), 7.56-7.50 (m, 1H), 7.09 (d, 1H), 6.99 (d, 1H), 4.82 (s, 2H), 4.50-4.41 (m, 2H), 4.02-3.89 (m, 2H), 2.81 (d, 3H); MS (EI) for C₂₆H₂₃N₃O₂: 410 (MH⁺).

N-ethyl-4-(4-quinolin-4-yl-2,3,4,5-tetrahydro-1,4-benzoxazepin-7-yl)benzamide. Prepared according to the method of example 12 using 4-chloroquinoline in step 3 and ethylamine in step 6. ¹H NMR (400 MHz, DMSO-d6); δ 8.61 (d, 1H), 8.55-8.50 (m, 1H), 8.02-7.90 (m, 4H), 7.81-7.76 (m, 3H), 7.72-7.62 (m, 2H), 7.50-7.45 (m, 1H), 7.12 (d, 1H), 7.00 (d, 1H), 4.68 (s, 2H), 4.43-4.37 (m, 2H), 3.86-3.80 (m, 2H), 1.14 (t, 3H); MS (EI) for C₂₇H₂₅N₃O₂: 424 (MH⁺).

N-propyl-4-(4-quinolin-4-yl-2,3,4,5-tetrahydro-1,4-benzoxazepin-7-yl)benzamide. Prepared according to the method of example 12 using 4-chloroquinoline in step 3 and propylamine in step 6. ¹H NMR (400 MHz, DMSO-d6); δ 8.61 (d, 1H), 8.54-8.48 (m, 1H), 8.03 (d, 1H), 7.98-7.91 (m, 3H), 7.84-7.77 (m, 3H), 7.75-7.68 (m, 1H), 7.68-7.62 (m, 1H), 7.55-7.48 (m, 1H), 7.10 (d, 1H), 7.00 (d, 1H), 4.75 (s, 2H), 4.46-4.39 (m, 2H), 3.93-3.85 (m, 2H), 3.28-3.20 (m, 2H), 1.61-1.49 (m, 2H), 0.91 (t, 3H); MS (EI) for C₂₈H₂₇N₃O₂: 438 (MH⁺).

3-{4-[2-methyl-7-(methyloxy)quinazolin-4-yl]-2,3,4,5-tetrahydro-1,4-benzoxazepin-7-yl}benzoic acid. Prepared according to the method of example 12 using 3-(methoxycarbonyl)phenylboronic acid in step 1 and 4-chloro-7-methoxy-2-methylquinazoline in step 3 and omission of steps 5 and 6. ¹H NMR (400 MHz, DMSO-d6); δ 8.21 (m, 1H), 7.96-7.86 (m, 1H), 7.78-7.72 (m, 3H), 7.64-7.50 (m, 2H), 7.15-7.00 (m, 3H), 5.03 (s, 2H), 4.49-4.42 (m, 2H), 4.20-4.12 (m, 2H), 3.88 (s, 3H), 2.42 (s, 3H); MS (EI) for C₂₆H₂₃N₃O₄: 440 (M⁻).

N-methyl-3-{4-[2-methyl-7-(methyloxy)quinazolin-4-yl]-2,3,4,5-tetrahydro-1,4-benzoxazepin-7-yl}benzamide. Prepared according to the method of example 12 using 3-(methoxycarbonyl)phenylboronic acid in step 1,4-chloro-7-methoxy-2-methylquinazoline in step 3, and methylamine in step 6. ¹H NMR (400 MHz, DMSO-d6); δ 8.61-8.52 (m, 1H), 8.12-8.09 (m, 1H), 7.89 (d, 1H), 7.84-7.73 (m, 3H), 7.61-7.51 (m, 2H), 7.12-7.09 (m, 1H), 7.08-7.00 (m, 2H), 5.03 (s, 2H), 4.51-4.41 (m, 2H), 4.22-4.12 (m, 2H), 3.88 (s, 3H), 2.85-2.79 (d, 3H), 2.42 (s, 3H); MS (EI) for C₂₇H₂₆N₄O₃: 455 (MH⁺).

N-ethyl-3-{4-[2-methyl-7-(methyloxy)quinazolin-4-yl]-2,3,4,5-tetrahydro-1,4-benzoxazepin-7-yl}benzamide. Prepared according to the method of example 12 using 3-(methoxycarbonyl)phenylboronic acid in step 1,4-chloro-7-methoxy-2-methylquinazoline in step 3, and ethylamine in step 6. ¹H NMR (400 MHz, DMSO-d6); δ 8.64-8.57 (m, 1H), 8.14-8.08 (m, 1H), 7.97-7.89 (d, 1H), 7.84-7.75 (m, 3H), 7.61-7.51 (m, 2H), 7.12 (d, 1H), 7.08-7.02 (m, 2H), 5.06 (s, 2H), 4.53-4.43 (m, 2H), 4.23-4.14 (m, 2H), 3.88 (s, 3H), 2.44 (s, 3H), 1.15 (t, 3H); MS (EI) for C₂₈H₂₈N₄O₃: 469 (MH⁺).

### Example 13

### Methyl[6-(4-{5-[(4-fluorophenyl)methyl]-6-methylpyrimidin-4-yl}-2,3,4,5-tetrahydro. 1,4-benzoxazepin-7-yl)-1H-imidazo[4,5-b]pyridin-2-yl]carbamate and 6-(4-{5-[(4-fluorophenyl)methyl]-6-methylpyrimidin-4-yl}-2,3,4,5-tetrahydro-1,4-benzoxazepin-7-yl)-1H-imidazo[4,5-b]pyridin-2-amine

STEP 1: To a slurry of 1,1-dimethylethyl 7-(4,4,5,5-tetramethyl-1,3,2-dioxaborolan-2-yl)-2,3-dihydro-1,4-benzoxazepine-4(5H)-carboxylate (example 9, step 1) (3.3 g, 8.8 mmol) in methanol (25 mL) was added anhydrous hydrogen chloride (6.0 mL, 4 N in dioxane, 24 mmol). The reaction mixture was heated (60° C.) for 1.5 h and was concentrated. The resulting solid was suspended in ethyl ether (50 mL), collected by filtration and washed with ethyl ether (2×30 mL) to afford 7-(4,4,5,5-tetramethyl-1,3,2-dioxaborolan-2-yl)-2,3,4,5-tetrahydro-1,4-benzoxazepine hydrochloride (1.4 g, 50% yield) as a white solid. MS (EI) for C₁₅H₂₂BNO₃: 276 (MH⁺).

STEP 2: To a solution of 7-(4,4,5,5-tetramethyl-1,3,2-dioxaborolan-2-yl)-2,3,4,5-tetrahydro-1,4-benzoxazepine hydrochloride (1.4 g, 4.5 mmol) and DIPEA (6.4 mL, 37 mmol) in NMP (24 mL) was added and 4-chloro-5-(4-fluorobenzyl)-6-methylpyrimidine (reagent preparation 5) (1.7 g, 7.3 mmol). The resulting mixture was heated (120° C.) for 12 h and then partitioned between ethyl acetate (100 mL) and 1N aqueous hydrochloric acid (50 mL). The organic layer was washed with additional 1N aqueous hydrochloric acid (50 mL), brine (50 mL), dried over anhydrous magnesium sulfate, filtered and concentrated. Column chromatography on silica (0-30% ethyl acetate/hexanes) provided 4-{5-[(4-fluorophenyl)methyl]-6-methylpyrimidin-4-yl}-7-(4,4,5,5-tetramethyl-1,3,2-dioxaborolan-2-yl)-2,3,4,5-tetrahydro-1,4-benzoxazepine (1.7 g, 47% yield) as a white foam. MS (EI) for C₂₇H₃₁BFN₃O₃: 476 (MH⁺).

STEP 3: To a solution of 4-{5-[(4-fluorophenyl)methyl]-6-methylpyrimidin-4-yl}-7-(4,4,5,5-tetramethyl-1,3,2-dioxaborolan-2-yl)-2,3,4,5-tetrahydro-1,4-benzoxazepine (1.3 g, 2.7 mmol), potassium hydrogen carbonate (1.4 g, 14 mmol), 2-amino-5-bromo-3-nitropyridine (0.98 g, 4.5 mmol) and DIPEA (1.4 mL, 8.2 mmol) in dioxane (8 mL) and water (1 mL) was added dichloro[1,1-bis(diphenylphosphino]ferrocenepalladium (II) dichloromethane adduct (0.20 g, 0.27 mmol). The biphasic mixture was then heated (90° C.) for 2 h and the organic layer was separated and purified by column chromatography on silica (0-10% methanol/dichloromethane) to provide 5-(4-{5-[(4-fluorophenyl)methyl]-6-methylpyrimidin-4-yl}-2,3,4,5-tetrahydro-1,4-benzoxazepin-7-yl)-3-nitropyridin-2-amine (0.49 g, 37% yield) as a orange-red solid. MS (EI) for C₂₆H₂₃FN₆O₃: 487 (MH⁺).

STEP 4: To a solution of 5-(4-{5-[(4-fluorophenyl)methyl]-6-methylpyrimidin-4-yl}-2,3,4,5-tetrahydro-1,4-benzoxazepin-7-yl)-3-nitropyridin-2-amine (0.36 g, 0.75 mmol) in acetic acid (10 mL) was added tin (II) chloride (0.75 g, 3.8 mmol). The reaction mixture was heated (50° C.) for 2 h and then partitioned between ethyl acetate (10 mL) and 1 N aqueous sodium hydroxide (20 mL). The resulting mixture was filtered through Celite and the organic layer was washed with brine (10 mL), dried over anhydrous magnesium sulfate then filtered and concentrated. Column chromatography on silica (5% methanol/dichloromethane) provided 5-(4-{5-[(4-fluorophenyl)methyl]-6-methylpyrimidin-4-yl}-2,3,4,5-tetrahydro-1,4-benzoxazepin-7-yl)pyridine-2,3-diamine (0.21 g, 60% yield) as a pale yellow solid. MS (EI) for C₂₆H₂₅FN₆O: 457 (MH⁺).

STEP 5: To a solution of 5-(4-{5-[(4-fluorophenyl)methyl]-6-methylpyrimidin-4-yl}-2,3,4,5-tetrahydro-1,4-benzoxazepin-7-yl)pyridine-2,3-diamine (0.21 g, 0.45 mmol) in acetic acid (5 mL) was added 1,3-bis(methoxycarbonyl)-2-methyl-2-thiopseudourea (0.09 g, 0.45 mmol). The reaction mixture was heated (60° C.) for 12 h and then concentrated. The resulting residue was dissolved in acetonitrile and purified by preparative reverse phase HPLC to provide methyl[6-(4-{5-[(4-fluorophenyl)methyl]-6-methylpyrimidin-4-yl}-2,3,4,5-tetrahydro-1,4-benzoxazepin-7-yl)-1H-imidazo[4,5-b]pyridin-2-yl]carbamate (0.01 g, 4% yield) as a white solid. ¹H NMR (400 MHz, DMSO-D₆): δ 11.97 (bs, 1H), 8.51 (d, 1H), 8.28 (s, 1H), 7.79 (s, 1 h), 7.42-7.50 (m, 1H), 6.95-7.13 (m, 6H), 4.53 (s, 2H), 4.25-4.32 (m, 2H), 4.00 (s, 2H), 3.74-3.84 (m, 5H), 2.16 (s; 3H); MS (EI) for C₂₉H₂₆FN₇O₃: 540 (MH⁺).

STEP 6: A mixture of methyl[6-(4-{5-[(4-fluorophenyl)methyl]-6-methylpyrimidin-4-yl}-2,3,4,5-tetrahydro-1,4-benzoxazepin-7-yl)-1H-imidazo[4,5-b]pyridin-2-yl]carbamate (15 mg, 0.028 mmol) in methanol and 2 M aqueous potassium hydroxide (1:1, 2 mL) was stirred at 65° C. for 18 hours. The reaction mixture was cooled, adjusted to pH 10 with 2 N hydrochloric acid, concentrated, diluted with ethyl acetate (10 mL), washed with brine solution (5 mL), dried over sodium sulfate, filtered and concentrated. The residue was purified by preparative reverse phase HPLC to give 6-(4-{5-[(4-fluorophenyl)methyl]-6-methylpyrimidin-4-yl}-2,3,4,5-tetrahydro-1,4-benzoxazepin-7-yl)-1H-imidazo[4,5-b]pyridin-2-amine (8.9 mg, 67% yield). ¹H NMR (400 MHz, DMSO-D₆): 8.46 (1H), 8.02 (br, 1H), 7.47 (d, 1H), 7.39 (dd, 1H), 7.10 to 6.96 (m, 5H), 7.75 (br, 1H), 4.53 (s, 2H), 4.30 (m, 2H), 4.00 (s, 2H), 3.89 (m, 2H), 2.22 (s, 3H), MS (EI) for C₂₇H₂₄FN₇O: 428 (MH⁺).

Using analogous synthetic techniques and substituting with alternative starting reagents in step 2 the following compounds of the invention were prepared. Alternative starting materials were obtained commercially unless otherwise indicated.

6-[4-(6,6-dimethyl-5,6,7,8-tetrahydroquinazolin-4-yl)-2,3,4,5-tetrahydro-1,4-benzoxazepin-7-yl]-1H-imidazo[4,5-b]pyridin-2-amine. Synthesized as the dihydrochloride salt according to the method of example 13 using (4-{[(1,1-dimethylethyl)oxy]carbonyl}-2,3,4,5-tetrahydro-1,4-benzoxazepin-7-yl)boronic acid (example 1, step 2) in step 1 and with 4-chloro-6,6-dimethyl-5,6,7,8-tetrahydroquinazoline (reagent preparation 3) in step 2. ¹H NMR (400 MHz, DMSO-D₆): 8.68 (s, 1H), 8.62 (bs, 2H), 8.41 (s, 1H), 7.96 (s, 1 h), 7.78 (s, 1H), 7.57 (d, 1H), 7.06 (d, 1H), 5.07 (s, 2H), 4.43-4.51 (m, 2H), 4.13-4.22 (m, 1H), 2.74-2.84 (m, 2H), 2.53 (s, 2H), 1.53-1.62 (m, 2H), 0.86 (s, 6H); MS (EI) for C₂₅H₂₇N₇O: 442.4 (MH⁺).

6-[4-(6-bromoquinazolin-4-yl)-2,3,4,5-tetrahydro-1,4-benzoxazepin-7-yl]-1H-imidazo[4,5-b]pyridin-2-amine. Synthesized as the dihydrochloride salt according to the method of example 13 using (4-{[(1,1-dimethylethyl)oxy]carbonyl}-2,3,4,5-tetrahydro-1,4-benzoxazepin-7-yl)boronic acid (example 1, step 2) in step 1 and 6-bromo-4-chloroquinazoline in step 2. ¹H NMR (400 MHz, DMSO-D₆): δ 8.69 (s, 1H), 8.47 (s, 1H), 8.36 (s, 1H), 8.15 (dd, 1 h), 8.11 (s, 1H), 7.82 (s, 1H), 7.79 (d, 1H), 7.64 (d, 1H), 7.16 (s, 1H), 5.14 (s, 2H), 4.66-4.74 (m, 2H), 4.54-4.61 (m, 2H); MS (EI) for C₂₃H₁₈BrN

6-[4-(6-fluoroquinazolin-4-yl)-2,3,4,5-tetrahydro-1,4-benzoxazepin-7-yl]-1H-imidazol[4,5-b]pyridin-2-amine. Synthesized according to the method of example 13 using 4-chloro-6-fluoroquinazoline in step 2. ¹H NMR (400 MHz, CDCl₃): 8.68 (s, 1H), 8.39 (d, 1H), 8.10-7.84 (m, 4H), 7.79 (d, 1H), 7.59-7.54 (dd, 1H), 7.09 (d, 1H), 5.47 (s, 2H), 4.69-4.59 (m, 4H). MS (EI) for C₂₅H₂₂FN₇O₄: 428.1 (MH⁺).

6-[4-(6-chloroquinazolin-4-yl)-2,3,4,5-tetrahydro-1,4-benzoxazepin-7-yl]-1H-imidazo[4,5-b]pyridin-2-amine. Synthesized according to the method of example 13 using 4,6-dichloroquinazoline in step 2. ¹H NMR (400 MHz, MeOH): 8.55 (s, 1H), 8.31 (m, 1H), 8.09 (m, 1H), 7.83-7.79 (m, 3H), 7.68 (m, 1H), 7.55-7.52 (m, 1H), 7.13-7.09 (m, 1H), 5.08 (s, 2H), 4.55-4.50 (m, 2H), 4.30-4.25 (m, 2H); MS (EI) for C₂₃H₁₈ClN₇O: 444.1 (MH⁺).

6-(4-{5-[(4-fluorophenyl)methyl]-6-methylpyrimidin-4-yl}-2,3,4,5-tetrahydro-1,4-benzoxazepin-7-yl)-1H-imidazo[4,5-b]pyridin-2-amine. Prepared by a modification of the example 13 sequence starting with step 3 using 1,1-dimethylethyl 7-(4,4,5,5-tetramethyl-1,3,2-dioxaborolan-2-yl)-2,3-dihydro-1,4-benzoxazepine-4(5H)-carboxylate (example 9, step 1) and 2-amino-5-bromo-3-nitropyridine, followed by conducting steps 4 and 5, then 1. Subsequently step 2 was carried out using 4-chloro-5-(4-fluorobenzyl)-6-methylpyrimidine (reagent preparation 5) followed by step 6. ¹H NMR (400 MHz, DMSO-D₆): 8.46 (s, 1H), 8.02 (br, 1H), 7.47 (d, 1H), 7.39 (dd, 1H), 7.10 to 6.96 (m, 5H), 7.75 (br, 1H), 4.53 (s, 2H), 4.30 (m, 2H), 4.00 (s, 2H), 3.89 (m, 2H), 2.22 (s, 3H), MS (EI) for C₂₇H₂₄FN₇O: 428 (MH⁺).

6-[4-(6-ethyl-5,6,7,8-tetrahydroquinazolin-4-yl)-2,3,4,5-tetrahydro-1,4-benzoxazepin-7-yl]-1H-imidazo[4,5-b]pyridin-2-amine. Prepared by a modification of the example 13 sequence starting with step 3 using 1,1-dimethylethyl 7-(4,4,5,5-tetramethyl-1,3,2-dioxaborolan-2-yl)-2,3-dihydro-1,4-benzoxazepine-4(5H)-carboxylate (example 9, step 1) and 2-amino-5-bromo-3-nitropyridine, followed by conducting steps 4 and 5, then 1. Subsequently step 2 was carried out using 4-chloro-6-ethyl-5,6,7,8-tetrahydroquinazoline (reagent preparation 8) followed by step 6. ¹H NMR (400 MHz, Methanol-D₄): 8.39 (s, 1H), 8.18 (s, 1H), 7.61 (s, 1H), 7.54 (br, 1H), 7.40 (d, 1H), 7.02 (d, 1H), 4.72 (dd, 2H), 4.50 (m, 1H), 4.33 (m, 1H), 4.09 (m, 1H), 3.84 (dd, 1H), 2.88 (m, 1H), 2.76 (m, 1H), 2.57 (dd, 1H), 2.41 (dd, 1H), 1.95 (m, 1H), 1.46 (m, 1H), 1.36 (m, 2H), 0.88 (t, 3H). MS (EI) for C₂₅H₂₇N₇O: 442 (MH⁺).

6-[4-(7-methyl-5,6,7,8-tetrahydroquinazolin-4-yl)-2,3,4,5-tetrahydro-1,4-benzoxazepin-7-yl]-1H-imidazo[4,5-b]pyridin-2-amine. Prepared by a modification of the example 13 sequence starting with step 3 using 1,1-dimethylethyl 7-(4,4,5,5-tetramethyl-1,3,2-dioxaborolan-2-yl)-2,3-dihydro-1,4-benzoxazepine-4(5H)-carboxylate (example 9, step 1) and 2-amino-5-bromo-3-nitropyridine, followed by conducting steps 4 and 5, then 1. Subsequently step 2 was carried out using 4-chloro-7-methyl-5,6,7,8-tetrahydroquinazoline (reagent preparation 8) followed by step 6. ¹H NMR (400 MHz, DMSO-D₆): 8.31 (1H), 8.13 (br, 1H), 7.64 (d, 1H), 7.49 (br, 1H), 7.41 (dd, 1H), 7.02 (d, 2H), 4.76 (dd, 2H), 4.41 (m, 1H), 4.23 (m, 1H), 4.06 to 3.91 (m, 2H), 2.88 (m, 2H), 2.59 (dt, 1H), 2.30 (dd, 1H), 1.92 (m, 2H), 1.19 (m, 1H), 1.08 (d, 3H), MS (EI) for C₂₄H₂₅N₇O: 428 (MH⁺).

6-{4-[6,7-bis(methyloxy)quinazolin-4-yl]-2,3,4,5-tetrahydro-1,4-benzoxazepin-7-yl}-1H-imidazo[4,5-b]pyridin-2-amin. Prepared by a modification of the example 13 sequence starting with step 3 using 1,1-dimethylethyl 7-(4,4,5,5-tetramethyl-1,3,2-dioxaborolan-2-yl)-2,3-dihydro-1,4-benzoxazepine-4(5H)-carboxylate (example 9, step 1) and 2-amino-5-bromo-3-nitropyridine, followed by conducting steps 4 and 5, then 1. Subsequently step 2 was carried out using 4-chloro-6,7-dimethoxyquinazoline followed by step 6. ¹H NMR (400 MHz, Methanol-D₄): 8.46 (s, 1H), 8.19 (br, 1H), 7.69 (br, 1H), 7.64 (d, 1H), 7.50 (dd, 1H), 7.14 (s, 1H), 7.12 (s, 1H), 7.10 (d, 1H), 5.04 (s, 2H), 4.54 (m, 2H), 4.16 (m, 2H), 3.95 (s, 3H), 3.56 (s, 3H), MS (EI) for C₂₅H₂₃FN₇O₃: 470 (MH⁺).

6-{4-[6-(methyloxy)quinazolin-4-yl]-2,3,4,5-tetrahydro-1,4-benzoxazepin-7-yl}-1H-imidazo[4,5-b]pyridin-2-amine. Prepared by a modification of the example 13 sequence starting with step 3 using 1,1-dimethylethyl 7-(4,4,5,5-tetramethyl-1,3,2-dioxaborolan-2-yl)-2,3-dihydro-1,4-benzoxazepine-4(5H)-carboxylate (example 9, step 1) and 2-amino-5-bromo-3-nitropyridine, followed by conducting steps 4 and 5, then 1. Subsequently step 2 was carried out using 4-chloro-6-methoxyquinazoline followed by step 6. ¹H NMR (400 MHz, Methanol-D₄): 8.45 (s, 1H), 8.19 (br, 1H), 7.71 (br, 1H), 7.67 (br, 1H), 7.61 (br, 1H), 7.48 (dd, 1H), 7.42 (dd, 1H), 7.18 (d, 1H), 7.10 (d, 1H), 5.02 (s, 2H), 4.53 (m, 2H), 4.17 (m, 2H), 3.58 (s, 3H), MS (EI) for C₂₄H₂₁N₇O₂: 440 (MH⁺).

6-[4-(6-iodoquinazolin-4-yl)-2,3,4,5-tetrahydro-1,4-benzoxazepin-7-yl]-1H-imidazo[4,5-b]pyridin-2-amine. Prepared by a modification of the example 13 sequence starting with step 3 using 1,1-dimethylethyl 7-(4,4,5,5-tetramethyl-1,3,2-dioxaborolan-2-yl)-2,3-dihydro-1,4-benzoxazepine-4(5H)-carboxylate (example 9, step 1) and 2-amino-5-bromo-3-nitropyridine, followed by conducting steps 4 and 5, then 1. Subsequently step 2 was carried out using 4-chloro-6-iodoquinazoline followed by step 6. ¹H NMR (400 MHz, Methanol-D₄): 8.56 (s, 1H), 8.38 (br, 1H), 8.34 (br, 1H), 8.03 (dd, 1H), 7.81 (br, 1H), 7.65 (br, 1H), 7.54 (s, 1H), 7.52 (s, 1H), 7.11 (d, 1H), 5.01 (s, 2H), 4.51 (m, 2H), 4.21 (m, 2H), MS (EI) for C₂₃H₁₈N₇O: 536 (MH⁺).

6-{4-[7-bromo-6-(methyloxy)quinazolin-4-yl]-2,3,4,5-tetrahydro-1,4-benzoxazepin-7-yl}-1H-imidazo[4,5-b]pyridin-2-amine. Prepared by a modification of the example 13 sequence starting with step 3 using 1,1-dimethylethyl 7-(4,4,5,5-tetramethyl-1,3,2-dioxaborolan-2-yl)-2,3-dihydro-1,4-benzoxazepine-4(5H)-carboxylate (example 9, step 1) and 2-amino-5-bromo-3-nitropyridine, followed by conducting steps 4 and 5, then 1. Subsequently step 2 was carried out using 7-bromo-4-chloro-6-methoxyquinazoline (reagent preparation 1) followed by step 6. ¹H NMR (400 MHz, Methanol-D₄): 8.48 (s, 1H), 8.19 (br, 1H), 8.00 (br, 1H), 7.67 (br, 1H), 7.66 (br, 1H), 7.58 (dd, 1H), 7.17 (s, 1H), 7.10 (d, 1H), 5.07 (s, 2H), 4.55 (m, 2H), 4.19 (m, 2H), 3.59 (s, 3H), MS (EI) for C₂₄H₂₀BrN₇O₂: 518 (MH⁺).

6-[4-(6-bromo-7-chloroquinazolin-4-yl)-2,3,4,5-tetrahydro-1,4-benzoxazepin-7-yl]-1H-imidazo[4,5-b]pyridin-2-amine. Prepared by a modification of the example 13 sequence starting with step 3 using 1,1-dimethylethyl 7-(4,4,5,5-tetramethyl-1,3,2-dioxaborolan-2-yl)-2,3-dihydro-1,4-benzoxazepine-4(5H)-carboxylate (example 9, step 1) and 2-amino-5-bromo-3-nitropyridine, followed by conducting steps 4 and 5, then 1. Subsequently step 2 was carried out using 6-bromo-4,7-dichloroquinazoline (reagent preparation 1) followed by step 6. ¹H NMR (400 MHz, Methanol-D₄): 8.55 (s, 1H), 8.36 (s, 1H), 8.31 (br, 1H), 7.91 (br, 1H), 7.78 (br, 1H), 7.68 (br, 1H), 7.53 (dd, 1H), 7.10 (d, 1H), 5.05 (s, 2H), 4.52 (m, 2H), 4.25 (m, 2H), MS (EI) for C₂₃H₁₇BrClN₇O: 522 (MH⁺).

6-{4-[6,7-bis(methyloxy)quinolin-4-yl]-2,3,4,5-tetrahydro-1,4-benzoxazepin-7-yl}-1H-imidazo[4,5-b]pyridin-2-amine. Prepared by a modification of the example 13 sequence starting with step 3 using 1,1-dimethylethyl 7-(4,4,5,5-tetramethyl-1,3,2-dioxaborolan-2-yl)-2,3-dihydro-1,4-benzoxazepine-4(5H)-carboxylate (example 9, step 1) and 2-amino-5-bromo-3-nitropyridine, followed by conducting steps 4 and 5, then 1. Subsequently step 2 was carried out using 4-chloro-6,7-dimethoxyquinoline followed by step 6. ¹H NMR (400 Methanol-D₄): 8.51 (d, 1H), 8.19 (d, 1H), 7.68 (br, 1H), 7.61 (br, 1H), 7.51 (dd, 1H), 7.24 (s, 1H), 7.20 (s, 1H), 7.12 (d, 1H), 7.00 (d, 1H), 4.75 (s, 2H), 4.49 (m, 2H), 4.00 (s, 3H), 3.92 (m, 2H), 3.59 (s, 3H), MS (EI) for C₂₆H₂₄N₆O₃: 469 (MH⁺).

Methyl (6-{4-[(7S)-7-ethyl-5,6,7,8-tetrahydroquinazolin-4-yl]-2,3,4,5-tetrahydro-1,4-benzoxazepin-7-yl}-1H-imidazo[4,5-b]pyridin-2-yl)carbamate. Prepared according to the method of example 13 by using (4-{[(1,1-dimethylethyl)oxy]carbonyl}-2,3,4,5-tetrahydro-1,4-benzoxazepin-7-yl)boronic acid (example 8, step 1) in step 1 and (7S)-4-chloro-7-ethyl-5,6,7,8-tetrahydroquinazoline (reagent preparation 3) in step 2. ¹H NMR (400 MHz, Methanol-d₄): 8.44 (s, 1H), 8.34 (s, 1H), 7.88 (s, 1H), 7.60 (s, 1H), 7.47 (d, 1H), 7.05 (d, 1H), 4.72 (dd, 2H), 4.38 (m, 1H), 4.29 (m, 1H), 3.95 to 3.81 (m, 2H), 2.85 (m, 2H), 2.48 (m, 1H), 2.26 (m, 1H), 1.88 (m, 1H), 1.69 (m, 1H), 1.35 (m, 2H), 1.11 (m, 1H), 0.92 (t, 3H); MS (EI) for C₂₇H₂₉N₇O₂: 500 (MH⁺).

Methyl (6-{4-[(7S)-7-ethyl-2-methyl-5,6,7,8-tetrahydroquinazolin-4-yl]-2,3,4,5-tetrahydro-1,4-benzoxazepin-7-yl}-1H-imidazo[4,5-b]pyridin-2-yl)carbamate. Prepared according to the method of example 13 by using (4-{[(1,1-dimethylethyl)oxy]carbonyl}-2,3,4,5-tetrahydro-1,4-benzoxazepin-7-yl)boronic acid (example 8, step 1) in step 1 and (7S)-4-chloro-7-ethyl-2-methyl-5,6,7,8-tetrahydroquinazoline (reagent preparation 3) in step 2. ¹H NMR (400 MHz, Methanol-d₄): 8.42 (s, 1H), 7.95 (s, 1H), 7.58 (s, 1H), 7.45 (d, 1H), 7.06 (d, 1H), 4.62 (br, 2H), 4.44 (m, 1H), 4.19 (m, 1H), 4.06 (m, 1H), 3.95 (m, 1H), 3.88 (s, 3H), 2.88 (m, 1H), 2.78 (m, 1H), 2.61 (m, 1H), 2.39 (s, 3H), 2.28 (m, 1H), 1.97 (m, 1H), 1.74 (m, 1H), 1.41 (m, 2H), 1.16 (m, 1H), 0.98 (t, 3H); MS (EI) for C₂₈H₃₁N₇O₃: 514 (MH⁺).

Methyl[6-(4-{2-[(dimethylamino)methyl]-6,6-dimethyl-5,6,7,8-tetrahydroquinazolin-4-yl}-2,3,4,5-tetrahydro-1,4-benzoxazepin-7-yl)-1H-imidazo[4,5-b]pyridin-2-yl]carbamate. The dihydrochloride salt was prepared as in example 13 using 1-(4-chloro-6,6-dimethyl-5,6,7,8-tetrahydroquinazolin-2-yl)-N,N-dimethylmethanamine (reagent preparation 17) in step 2 and omission of step 6. ¹H NMR (400 MHz, CD₃OD) δ 8.70-8.54 (m, 2H), 8.02 (d, 1H), 7.61 (dt, 1H), 7.10 (d, 1H), 5.18 (s, 2H), 4.57-4.50 (m, 2H), 4.47 (s, 2H), 4.27-4.15 (m, 2H), 3.93 (s, 3H), 2.93-2.81 (m, 8H), 2.57 (s, 2H), 1.71 (t, 2H), 0.92 (s, 6H); MS (ES) for C₃₀H₃₆N₈O₃: 557.2 (MH⁺).

Methyl[6-(4-{2-[(dimethylamino)methyl]-6,6-dimethyl-5,6,7,8-tetrahydroquinazolin-4-yl}-2,3,4,5-tetrahydro-1,4-benzoxazepin-7-yl)-1H-benzimidazol-2-yl]carbamate. Prepared as in example 13 using 1-(4-chloro-6,6-dimethyl-5,6,7,8-tetrahydroquinazolin-2-yl)-N,N-dimethylmethanamine (reagent preparation 17) in step 2,4-bromo-2-nitroaniline in step 3 then omission of step 6. ¹H NMR (400 MHz, CD₃OD) δ 7.62 (d, 1H), 7.58 (d, 1H), 7.45 (td, 2H), 7.40 (dd, 1H), 7.01 (d, 1H), 4.76 (s, 2H), 4.37-4.31 (m, 2H), 4.03-3.96 (m, 2H), 3.85 (s, 3H), 3.65 (s, 2H), 2.79 (t, 2H), 2.50 (s, 2H), 2.37 (s, 6H), 1.68 (t, 2H), 0.91 (s, 6H); MS (ES) for C₃₁H₃₇N₇O₃: 556.2 (MH⁺).

### Example 14

### 6-(4-{5-[(4-Fluorophenyl)methyl]-6-methylpyrimidin-4-yl}-2,3,4,5-tetrahydro-1,4-benzoxazepin-7-yl)-1H-benzimidazol-2-amine

STEP 1: A mixture of methyl[6-(4-{5-[(4-fluorophenyl)methyl]-6-methylpyrimidin-4-yl}-2,3,4,5-tetrahydro-1,4-benzoxazepin-7-yl)-1H-benzimidazol-2-yl]carbamate (example 2) (60 mg, 0.11 mmol) in methanol and 2M potassium hydroxide (1:1, 2 mL) was stirred at 65° C. for 18 hours. The reaction mixture was cooled, adjusted to pH 10 with 2M hydrochloric acid, concentrated, diluted with ethyl acetate (10 mL), washed with brine solution (5 mL), dried over sodium sulfate, filtered, concentrated. The residue was purified by preparative reverse phase HPLC to give 6-(4-{5-[(4-fluorophenyl)methyl]-6-methylpyrimidin-4-yl}-2,3,4,5-tetrahydro-1,4-benzoxazepin-7-yl)-1H-benzimidazol-2-amine (29 mg, 54% yield). ¹H NMR (400 MHz, Methanol-d₄): 8.44 (1H), 7.37 (dd, 1H), 7.31 (s, 1H), 7.29 (s, 1H), 7.14 (dd, 1H), 7.07-7.01 (m, 2H), 6.99 to 6.91 (m, 3H), 6.70 (br, 1H), 4.49 (s, 2H), 4.26 (m, 2H), 3.95 (s, 2H), 3.86 (m, 2H), 2.19 (s, 3H), MS (EI) for C₂₈H₂₅FN₆O: 481 (MH⁺).

Using analogous synthetic techniques and substituting with alternative starting reagents in step 1 the following compounds of the invention were prepared. Alternative starting materials were obtained commercially unless otherwise indicated.

6-{4-[6,7-bis(methyloxy)quinolin-4-yl]-2,3,4,5-tetrahydro-1,4-benzoxazepin-7-yl}-1H-benzimidazol-2-amine. Prepared according to the method of example 14 by using methyl (6-{4-[6,7-bis(methyloxy)quinolin-4-yl]-2,3,4,5-tetrahydro-1,4-benzoxazepin-7-yl}-1H-benzimidazol-2-yl)carbamate (example 2). ¹H NMR (400 MHz, Methanol-D₄): 8.51 (d, 1H), 8.19 (d, 1H), 7.68 (br, 1H), 7.61 (br, 1H), 7.51 (dd, 1H), 7.24 (s, 1H), 7.20 (s, 1H), 7.12 (d, 1H), 7.00 (d, 1H), 4.75 (s, 2H), 4.49 (m, 2H), 4.00 (s, 3H), 3.92 (m, 2H), 3.59 (s, 3H), MS (EI) for C₂₇H₂₅N₅O₃: 468 (MH⁺).

6-[4-(6,6-dimethyl-5,6,7,8-tetrahydroquinazolin-4-yl)-2,3,4,5-tetrahydro-1,4-benzoxazepin-7-yl]-1H-benzimidazol-2-amine. Prepared according to the method of example 14 by using methyl{6-[4-(6,6-dimethyl-5,6,7,8-tetrahydroquinazolin-4-yl)-2,3,4,5-tetrahydro-1,4-benzoxazepin-7-yl]-1H-benzimidazol-2-yl}carbamate (example 27). ¹H NMR (400 MHz, d₆-DMSO): 8.70 (s, 1H), 8.56 (s, 2H), 7.68 (m, 1H), 7.58 (s, 1H), 7.51-7.47 (m, 2H), 7.01 (d, 1H), 5.08 (br s, 2H), 4.47 (m, 2H), 4.19 (m, 2H), 2.79 (t, 2H), 2.54 (s, 2H), 1.73 (s, 3H), 1.58 (t, 2H), 0.84 (s, 6H); MS (EI) for C₂₆H₂₈N₆O: 441 (MH⁺).

### Example 15

### N-(2-Fluoroethyl)-5-(4-{5-[(4-fluorophenyl)methyl]-6-methylpyrimidin-4-yl}-2,3,4,5-tetrahydro-1,4-benzoxazepin-7-yl)-1H-benzimidazol-2-amine

STEP 1: 2-Fluoroethylamine hydrochloride salt (282.4 mg, 2.83 mmol) was suspended in 1:1 THF:DCM (6 mL) followed by addition of DIPEA (2.5 mL, 14.35 mmol). The mixture was cooled to 0° C. followed by slow addition of thiophosgene (217 uL, 2.8 mmol) by syringe over five minutes then allowed to slowly warm to room temperature over 30 minutes. 4-Bromobenzene-1,2-diamine (530 mg, 2.8 mmol) was then added and the reaction mixture was allowed to stir at room temperature over an additional 12 h. The mixture was concentrated and the residue partitioned with ethyl acetate and 10% aqueous citric acid. The organic phase was washed twice with additional 10% aqueous citric acid then brine, dried over anhydrous sodium sulfate, filtered and concentrated. The crude mixture of thiourea thus obtained was taken into THF (15 mL) followed by addition of mercury (II) oxide (640 mg, 2.95 mmol). The mixture was brought to reflux for 6 h then stirred an additional 60 h at room temperature. The crude mixture was filtered through a bed of celite with ethyl acetate washing and the filtrate concentrated then taken back into ethyl acetate. The organic solution was washed once with 1 M aqueous hydrochloric acid and the organic phase discarded. The aqueous phase was filtered to remove trace insoluble residue and the filtrate basified to pH 9-10 by dropwise addition of 50% aqueous sodium hydroxide. The aqueous phase was then extracted once with ethyl acetate and the organic solution was washed with brine then dried over anhydrous sodium sulfate, filtered and concentrated to afford crude 5-bromo-N-(2-fluoroethyl)-1H-benzo[d]imidazol-2-amine (390 mg, 53% yield) which was carried forward without further purification. MS (EI) for C₉H₉BrFN₃: 258, 260 (MH⁺).

STEP 2: 5-bromo-N-(2-fluoroethyl)-1H-benzo[d]imidazol-2-amine (390 mg, 1.51 mmol) thus obtained in step 1 was taken into THF (15 mL) followed by addition of DIPEA (600 uL, 3.4 mmol) and isobutyl chloroformate (400 uL, 3.06 mmol) and the mixture was stirred at room temperature for 1 h. The mixture was concentrated and the residue partitioned with ethyl acetate and 10% aqueous citric acid. The organic phase was washed with brine, dried over anhydrous sodium sulfate, filtered and concentrated. The residue was purified by silica gel chromatography to afford isobutyl 5-bromo-2-(2-fluoroethylamino)-1H-benzo[d]imidazole-1-carboxylate (290 mg, 54% yield) as a colorless crystalline solid. MS (EI) for C₁₄H₁₇BrFN₃O₂: 358, 360 (MH⁺).

STEP 3: Isobutyl 5-bromo-2-(2-fluoroethylamino)-1H-benzo[d]imidazole-1-carboxylate (76 mg, 0.21 mmol) and (4-{5-[(4-fluorophenyl)methyl]-6-methylpyrimidin-4-yl}-2,3,4,5-tetrahydro-1,4-benzoxazepin-7-yl)boronic acid (example 8 step 2) (100 mg, 0.25 mmol), and [1,1′-Bis(diphenylphosphino)ferrocene]dichloropalladium(II) (8.5 mg, 0.01 mmol) were taken into dioxane (1 mL) and water (200 uL) followed by addition of DIPEA (180 uL, 1.03 mmol) and the mixture was heated to 95° C. over 12 h. On cooling to room temperature the crude mixture was diluted with ethyl acetate and dried over anhydrous sodium sulfate then filtered through a bed of silica gel. The filtrate was concentrated and the residue taken into methanol (5 mL) and basified by addition of 5 drops of 50% aqueous sodium hydroxide. The methanol solution was stirred for 0.5 h at room temperature then concentrated. The residue was partitioned with isopropyl acetate and 1M aqueous sodium hydroxide. The organic phase was washed twice with additional 1M aqueous sodium hydroxide then dried over anhydrous sodium sulfate, filtered and concentrated. The residue was purified by silica gel chromatography using 20:1 ethyl acetate:ethanol then 10% methanol in dichloromethane as eluent. Fractions containing pure material were concentrated and the residue triturated with ethyl ether. The suspension collected by filtration to give N-(2-fluoroethyl)-5-(4-{5-[(4-fluorophenyl)methyl]-6-methylpyrimidin-4-yl}-2,3,4,5-tetrahydro-1,4-benzoxazepin-7-yl)-1H-benzimidazol-2-amine (27.4 mg, 25% yield). ¹H NMR (400 MHZ, DMSO-d₆): 10.84 (br s, 1H), 8.50 (s, 1H), 7.34 (d, 1H), 7.26 (br s, 1H), 7.18 (d, 1H), 7.12 (d, 4H), 6.98-6.91 (m, 3H), 6.85 (s, 1H), 4.68 (m, 1H), 4.55 (m, 1H), 4.46 (s, 2H), 4.25 (br s, 2H), 4.01 (s, 2H), 3.77 (br s, 2H), 2.16 (s, 3H). MS (EI) for C₃₀H₂₈F₂N₆O: 528 (MH⁺).

Using analogous synthetic techniques and substituting with alternative starting reagents in step 1 and conducting protecting group introduction/removal in steps 2 and 3 as required according to literature techniques appropriate for a given protecting group (see for example: Greene and Wuts, Protective Groups in Organic Synthetic, Wiley-Interscience) the following compounds of the invention were prepared. Alternative starting materials were obtained commercially unless otherwise indicated.

N-Ethyl-6-(4-{5-[(4-fluorophenyl)methyl]-6-methylpyrimidin-4-yl}-2,3,4,5-tetrahydro-1,4-benzoxazepin-7-yl)-1H-benzimidazol-2-amine. Synthesized according to the method of example 15 using ethyl isothiocyanate in step 1. ¹H NMR (400 MHz, d₆-DMSO): 10.78 (s, 1H), 8.50 (s, 1H), 7.38 (br s, 1H), 7.24 (br d, 1H), 7.14 (d, 6H), 6.98 (d, 1H), 6.86 (br s, 1H), 6.60 (br s, 1H), 4.46 (s, 2H), 4.25 (m, 2H), 4.02 (s, 2H), 3.77 (m, 2H), 3.32 (dd, 2H), 2.16 (s, 3H), 1.19 (t, 3H). MS (EI) for C₃₀H₂₉FN₆O: 509 (MH⁺).

6-[4-(6,6-dimethyl-5,6,7,8-tetrahydroquinazolin-4-yl)-2,3,4,5-tetrahydro-1,4-benzoxazepin-7-yl]-N-(2-fluoroethyl)-1H-benzimidazol-2-amine. Synthesized according to the method of example 15 using [4-(6,6-dimethyl-5,6,7,8-tetrahydroquinazolin-4-yl)-2,3,4,5-tetrahydro-1,4-benzoxazepin-7-yl]boronic acid (reagent preparation 23) in step 3. ¹H NMR (400 MHZ, DMSO-d₆): 9.41 (tr, 1H), 8.71 (s, 1H), 7.69 (s, 1H), 7.60 (s, 1H), 7.53-7.45 (m, 3H), 7.01 (d, 1H), 5.09 (s, 2H), 4.73 (tr, 1H), 4.62 (tr, 1H), 4.47 (s, 2H), 4.20 (s, 2H), 3.86 (q, 1H), 3.79 (q, 1H), 2.54 (s, 2H), 1.57 (tr, 2H), 0.86 (s, 6H); MS (EI) for C₂₈H₃₁FN₆O: 487 (MH⁺).

6-[4-(6,6-dimethyl-5,6,7,8-tetrahydroquinazolin-4-yl)-2,3,4,5-tetrahydro-1,4-benzoxazepin-7-yl]-N-ethyl-1H-benzimidazol-2-amine. Prepared according to the method of example 15 by using ethyl isothiocyanate in step 1 and [4-(6,6-dimethyl-5,6,7,8-tetrahydroquinazolin-4-yl)-2,3,4,5-tetrahydro-1,4-benzoxazepin-7-yl]boronic acid (reagent preparation 23) in step 3. ¹H NMR (400 MHz, d₆-DMSO): 8.37 (s, 1H), 7.56 (d, 1), 7.43 (dd, 1H), 7.38 (s, 1H), 7.20 (s, 2H), 7.00 (d, 1H), 4.62 (s, 2H), 4.29 (m, 2H), 3.83 (m, 2H), 3.35 (q, 2H), 2.70 (t, 2H), 2.47 (s, 2H), 1.60 (t, 2H), 1.19 (t, 3H), 0.86 (s, 6H); MS (EI) for C₂₈H₃₂N₆O: 469 (MH⁺).

### Example 16

### 5-(4-{5-[(4-fluorophenyl)methyl]-6-methylpyrimidin-4-yl}-2,3,4,5-tetrahydro-1,4-benzoxazepin-7-yl)-3H-imidazo[4,5-b]pyridin-2-amine

STEP 1: To a solution of (4-{5-[(4-fluorophenyl)methyl]pyrimidin-4-yl}-2,3,4,5-tetrahydro-1,4-benzoxazepin-7-yl)boronic acid (example 8, step 2) (0.200 g, 0.509 mmol) and 2-amino-6-chloro-3-nitropyridine (0.106 g, 0.610 mmol) in dioxane (3 mL) and water (0.4 ml) was added potassium carbonate (0.211 g, 1.53 mmol). The solution was sparged with N₂(g) for five minutes before the addition of dichloro[1,1-bis(diphenylphosphino]ferrocenepalladium (II) dichloromethane adduct (0.042 g, 10 mol %). The resulting suspension was heated at 90° C. for 20 h in a sealed tube vessel. On cooling to room temperature the mixture was diluted with acetonitrile (25 mL) then filtered and concentrated to afford 6-(4-{5-[(4-fluorophenyl)methyl]pyrimidin-4-yl}-2,3,4,5-tetrahydro-1,4-benzoxazepin-7-yl)-3-nitropyridin-2-amine (0.247 g, 100% yield) as an oil that was used without further purification. MS (EI) for C₂₆H₂₃FN₆O₃: 487 (MH⁺).

STEP 2: To a solution 6-(4-{5-[(4-fluorophenyl)methyl]pyrimidin-4-yl}-2,3,4,5-tetrahydro-1,4-benzoxazepin-7-yl)-3-nitropyridin-2-amineas obtained in step 1 (0.247 g, 0.508) in ethanol (20 mL) was added 10% palladium on carbon (0.200 g) and glacial acetic acid (1 mL). The solution was sparged with N₂(g) for five minutes then hydrogenated using a Parr apparatus for 1 hour under H₂(g) at 40 psi. Filtration and concentration afforded a brown residue that was purified by silica gel chromatography (9:1 dichloromethane/methanol) to provide 6-(4-{5-[(4-fluorophenyl)methyl]pyrimidin-4-yl}-2,3,4,5-tetrahydro-1,4-benzoxazepin-7-yl)pyridine-2,3-diamine (0.200 g, 86%) as a light brown oil. MS (EI) for C₂₆H₂₅FN₆O: 457 (MH⁺).

STEP 3: To a solution of 6-(4-{5-[(4-fluorophenyl)methyl]pyrimidin-4-yl}-2,3,4,5-tetrahydro-1,4-benzoxazepin-7-yl)pyridine-2,3-diamine (0.200 g, 0.438 mmol) in glacial acetic acid (3 mL) was added 1,3-bis(methoxycarbonyl)-2-methyl-2-thiopseudourea (0.117 g, 0.570 mmol). The reaction mixture was stirred at 80° C. for 2 h and then concentrated. Ethyl acetate (100 mL) was added to the residue, and the solution was washed with saturated sodium bicarbonate (50 mL) then dried over anhydrous sodium sulfate. Filtration and concentration afforded a brown residue that was taken up in diethyl ether (10 mL) and the resulting precipitate was collected by filtration to give methyl[5-(4-{5-[(4-fluorophenyl)methyl]pyrimidin-4-yl}-2,3,4,5-tetrahydro-1,4-benzoxazepin-7-yl)-3H-imidazo[4,5-b]pyridine-2-yl]carbamate (0.70 g, 30% yield) as a brown solid. MS (EI) for C₂₆H₂₅FN₆O: 457 (MH⁺).

STEP 4: To a solution of methyl[5-(4-{5-[(4-fluorophenyl)methyl]pyrimidin-4-yl}-2,3,4,5-tetrahydro-1,4-benzoxazepin-7-yl)-3H-imidazo[4,5-b]pyridine-2-yl]carbamate (0.70 g, 0.129 mmol) in methanol (3 mL) was added 2 M aqueous potassium hydroxide (3 mL) and the mixture was heated at 100° C. for 1 hour. After cooling to room temperature the reaction mixture was concentrated and then diluted with water (5 mL) and the pH adjusted to 9 with 1 M hydrochloric acid. The resulting precipitate was collected by filtration, dissolved in a minimum of methanol and purified by preparative reverse phase HPLC to afford 5-(4-{5-[(4-fluorophenyl)methyl]-6-methylpyrimidin-4-yl}-2,3,4,5-tetrahydro-1,4-benzoxazepin-7-yl)-3H-imidazo[4,5-b]pyridin-2-amine (0.0283 g, 46% yield. ¹H NMR (400 MHz, methanol-d₄): 8.45 (s, 1H), 7.75 (d, 1H), 7.51 (d, 1H), 7.17 (d, 1H), 7.11-7.06 (m, 3H), 7.01-6.96 (m, 3H), 4.55 (s, 2H), 4.30 (t, 2H), 4.00 (s, 2H), 3.89 (t, 2H), 2.22 (s, 3H); MS (EI) for C₂₇H₂₄FN₇O: 482 (MH⁺)

### Example 17

### N,N-dimethyl-6-(4-quinolin-4-yl-2,3,4,5-tetrahydro-1,4-benzoxazepin-7-yl)-1H-benzimidazol-2-amine

STEP 1: A solution of 4-(4-quinolin-4-yl-2,3,4,5-tetrahydrobenzoxazepin-7-yl)benzene-1,2-diamine (example 2) (50 mg, 0.13 mmol), N-(chloro(dimethylamino)methylene)-N-methylmethanaminium hexafluorophosphate (44 mg, 0.16 mmol) and N-methylmorpholine (0.20 g, 1.9 mmol) in N,N-dimethylformamide (10 mL) was heated to 140° C. for 1 hour. After cooling the reaction was diluted with ethyl acetate (80 mL), and washed twice with water (2×20 mL) and brine (20 mL). The solution was dried over anhydrous sodium sulfate, filtered and concentrated to a solid residue. The residue was then taken into N,N-dimethylformamide (3 mL), then diluted with chloroform (15 mL). The resulting solid was collected by filtration and the filter cake was precipitated once more using the same technique to give N,N-dimethyl-6-(4-quinolin-4-yl-2,3,4,5-tetrahydro-1,4-benzoxazepin-7-yl)-1H-benzimidazol-2-amine (27 mg, 48% yield) as a gray powder. ¹H NMR (400 MHz, d6-DMSO): 8.56 (d, 1H), 8.30 (d, 1H), 8.06-7.97 (m, 2H), 7.90 (br s, 1H), 7.72 (t, 1H), 7.62 (br s, 1H), 7.56 (m, 2H), 7.48 (d, 1H), 6.99 (dd, 2H), 5.28 (s, 2H), 4.61 (s, 2H), 4.38 (s, 2H), 3.28 (s, 6H); MS (EI) for C₂₇H₂₅N₅O: 436 (MH⁺).

### Example 18

### 7-(2-cyclopropyl-1H-benzimidazol-6-yl)-4-quinolin-4-yl-2,3,4,5-tetrahydro-1,4-benzoxazepine

STEP 1: To cyclopropanecarboxylic acid (33 mg, 0.39 mmol) in N,N-dimethylformamide (10 mL) added O-benzotriazole-N,N,N′,N′-tetramethyluronium hexafluorophosphate (0.15 g, 0.40 mmol) and the mixture stirred at 25° C. for 30 minutes. 4-(4-quinolin-4-yl-2,3,4,5-tetrahydrobenzoxazepin-7-yl)benzene-1,2-diamine (example 2) (100 mg, 0.26 mmol) was added followed by and N-methylmorpholine (66 uL, 0.60 mmol) and stirred 18 hours at 25° C. The reaction was diluted with ethyl acetate (80 mL), and washed with 2M aqueous sodium hydroxide (40 mL), water (40 mL) and brine (20 mL). The organic phase was dried over anhydrous sodium sulfate, filtered and concentrated. The residue was purified by silica gel column chromatography (methanol/ethyl acetate, 1:8). This afforded N-(2-amino-5-(4-quinolin-4-yl-2,3,4,5-tetrahydrobenzoxazepin-7-yl)phenyl)cyclopropanecarboxamide (55 mg, 47% yield). MS (EI) for C₂₈H₂₆N₄O₂: 451 (MH⁺).

STEP 2: As solution of N-(2-amino-5-(4-quinolin-4-yl-2,3,4,5-tetrahydrobenzoxazepin-7-yl)phenyl)cyclopropanecarboxamide (55 mg, 0.12 mmol) in acetic acid (10 mL) was heated to 110° C. for 4 hours. The reaction mixture was then cooled and concentrated. The residue was chromatographed on silica gel using (methanol/ethyl acetate, 1:10) as eluent. Product containing fractions were concentrated and the residue purified by preparative reverse phase HPLC (0.1% trifluoroacetic acid buffered aqueous acetonitrile mobile phase) to give 7-(2-cyclopropyl-1H-benzimidazol-6-yl)-4-quinolin-4-yl-2,3,4,5-tetrahydro-1,4-benzoxazepine (16.6 mg, 32% yield) as the trifluoroacetic acid salt. ¹H NMR (400 MHz, d₆-DMSO): 8.57 (d, 1H), 8.31 (d, 1H), 7.98 (m, 3H), 7.88 (s, 1H), 7.70 (m, 3H), 7.61 (dd, 1H), 6.99 (dd, 2H), 5.30 (br s, 2H), 4.62 (br s, 2H), 4.41 (br s, 2H), 2.46 (m, 1H), 1.36 (m, 4H); MS (EI) for C₂₈H₂₄N₄O: 433 (MH⁺).

Using analogous synthetic techniques and substituting with alternative starting reagents in step 1 the following compounds of the invention were prepared. Alternative starting materials were obtained commercially unless otherwise indicated.

7-{2-[(methyloxy)methyl]-1H-benzimidazol-6-yl}-4-quinolin-4-yl-2,3,4,5-tetrahydro-1,4-benzoxazepine. Synthesized according to the method of example 18 using methoxyacetic acid in step 1. ¹H NMR (400 MHz, d₆-DMSO): 8.57 (d, 1H), 8.33 (d, 1H), 7.96 (m, 3H), 7.89 (s, 1H), 7.68 (m, 3H), 7.61 (d, 1H), 6.98 (dd, 2H), 5.33 (s, 2H), 4.81 (s, 2H) 4.58 (br s, 2H), 4.39 (br s, 2H), 3.47 (s, 3H); MS (EI) for C₂₇H₂₄N₄O₂: 437 (MH⁺).

7-(2-propyl-1H-benzimidazol-6-yl)-4-quinolin-4-yl-2,3,4,5-tetrahydro-1,4-benzoxazepine. Synthesized according to the method of example 18 using butyric acid in step 1. ¹H NMR (400 MHz, d₆-DMSO): 8.62 (d, 1H), 8.03 (d, 1H), 7.94 (d, 1H), 7.68 (m, 3H), 7.58 (d, 1H), 7.50 (t, 1H), 7.42 (m, 1H), 7.11 (d 1H), 7.02 (d, 1H), 4.61 (s, 2H) 4.38 (br s, 2H), 4.82 (br s, 2H), 2.81 (t, 2H), 1.82 (q, 2H), 0.95 (t, 3H)); MS (EI) for C₂₈H₂₆N₄O: 435 (MH⁺).

7-(2-cyclopentyl-1H-benzimidazol-6-yl)-4-quinolin-4-yl-2,3,4,5-tetrahydro-1,4-benzoxazepine. Synthesized according to the method of example 18 using cyclopentanecarboxylic acid in step 1. ¹H NMR (400 MHz, d₆-DMSO): 8.61 (d, 1H), 8.04 (d, 1H), 7.95 (d, 1H), 7.70 (m, 2H), 7.59-7.41 (m, 4H), 7.11 (d, 1H), 7.04 (d, 1H), 4.63 (s, 2H) 4.38 (br s, 2H), 3.82 (br s, 2H), 2.11 (m, 2H), 1.90 (m, 2H), 1.79 (m, 2H), 1.67 (m, 2H); MS (EI) for C₃₀H₂₈N₄O: 462 (MI).

7-(2-cyclohexyl-1H-benzimidazol-6-yl)-4-quinolin-4-yl-2,3,4,5-tetrahydro-1,4-benzoxazepine. Synthesized according to the method of example 18 using cyclohexanecarboxylic acid in step 1. ¹H NMR (400 MHz, d₆-DMSO): 8.62 (d, 1H), 8.03 (d, 1H), 7.95 (m, 3H), 7.68 (m, 3H), 7.63 (m, 3H), 7.42 (d, 1H), 7.10 (d, 1H), 7.03 (d, 1H), 4.63 (s, 2H), 4.39 (br s, 2H), 3.81 (br s, 2H), 2.84 (m, 1H), 2.03 (d, 2H), 1.89-1.74 (m, 8H), 1.63-1.25 (m, 2H); MS (EI) for C₃₁H₃₀N₄O: 475 (MH⁺).

7-(2-azetidin-3-yl-1H-benzimidazol-6-yl)-4-quinolin-4-yl-2,3,4,5-tetrahydro-1,4-benzoxazepine. Synthesized according to the method of example 18 using N—BOC azetidine-3-carboxylic acid in step 1. ¹H NMR (400 MHz, d₆-DMSO): 8.96 (br d, 2H), 8.61 (d, 1H), 7.55 (d, 1H), 7.95 (m, 3H), 7.88 (s, 1H), 7.70 (m, 2H), 7.60 (m, 1H), 7.02 (d, 2H), 5.30 (s, 2H), 4.62 (br s, 2H), 4.22 (br s, 2H), 4.38 (br s, 5H); MS (EI) for C₂₈H₂₅N₅O: 448 (MH⁺).

7-(2-piperidin-2-yl-1H-benzimidazol-6-yl)-4-quinolin-4-yl-2,3,4,5-tetrahydro-1,4-benzoxazepine. Synthesized according to the method of example 18 using racemic N—BOC pipecolinic acid in step 1. ¹H NMR (400 MHz, d₆-DMSO): 9.96 (br d, 1H), 9.37 (m, 1H), 8.58 (d, 1H), 8.35 (d, 1H), 7.98 (m, 3H), 7.90 (s, 1H), 7.77-7.58 (m, 4H), 7.00 (m, 2H), 5.30 (s, 2H), 4.62 (m, 3H) 4.42 (br s, 2H), 3.41 (d, 1H), 3.13 (m, 1H), 2.42 (m, 2H), 0.80 (m, 5H); MS (EI) for C₃₁H₃₃N₅O: 492 (MH⁺).

7-[2-(1-methylethyl)-1H-benzimidazol-6-yl]-4-quinolin-4-yl-2,3,4,5-tetrahydro-1,4-benzoxazepine. Synthesized according to the method of example 18 using isobutyric acid in step 1. ¹H NMR (400 MHz, d₆-DMSO): 8.59 (d, 1H), 8.35 (d, 1H), 7.97 (m, 3H), 7.91 (br s, 1H), 7.75 (br s, 2H), 7.68 (m, 1H), 7.61 (m, 1H), 7.00 (dd, 2H), 5.29 (s, 2H), 4.62 (br s, 3H) 4.40 (br s, 2H), 3.43 (m, 1H), 1.44 (d, 6H); MS (EI) for C₂₉H₃₀N₄O: 451 (MH⁺).

4-(6,6-dimethyl-5,6,7,8-tetrahydroquinazolin-4-yl)-7-(2-ethyl-1H-benzimidazol-6-yl)-2,3,4,5-tetrahydro-1,4-benzoxazepine. Prepared according to the method of example 18 omitting step 1 and by using 4-[4-(6,6-dimethyl-5,6,7,8-tetrahydroquinazolin-4-yl)-2,3,4,5-tetrahydro-1,4-benzoxazepin-7-yl]benzene-1,2-diamine and trimethyl orthopropionate in step 2. ¹H NMR (400 MHz, d₆-DMSO): 8.69 (s, 1H), 7.94 (s, 1H), 7.84-7.78 (m, 3H), 7.60 (m, 1H), 7.35 (s, 1H), 7.23 (s, 1H), 7.04 (d, 1H), 5.09 (s, 2H), 4.48 (m, 2H), 4.19 (m, 2H), 3.18 (dd, 2H), 2.70 (t, 2H), 2.54 (s, 2H), 1.58 (t, 2H), 1.45 (t, 3H), 0.85 (s, 6H); MS (EI) for C₂₈H₃₁N₅O: 454 (MH⁺).

### Example 19

### 7-[2-(methylthio)-1H-benzimidazol-6-yl]-4-quinolin-4-yl-2,3,4,5-tetrahydro-1,4-benzoxazepine

STEP 1: To a solution of 4-(4-quinolin-4-yl-2,3,4,5-tetrahydro-1,4-benzoxazepin-7-yl)-benzene-1,2-diamine (630 mg, 1.65 mmol, synthesized according to the method of example 2 step) in tetrahydrofuran (30 mL) was added 1,1′-thiocarbonyldiimidazole (587 mg, 3.29 mmol), and the reaction mixture was stirred at room temperature for 20 h. The mixture was concentrated and the residue crystallized from methanol to give 5-(4-quinolin-4-yl-2,3,4,5-tetrahydro-1,4-benzoxazepin-7-yl)-1,3-dihydro-2H-benzimidazole-2-thione (357 mg, 51% yield) as a brown solid. ¹H NMR (400 MHz, DMSO-d₆): 12.74 (s, 1H), 12.66 (s, 1H), 8.57 (d, 1H), 8.28 (d, 1H), 8.00 (d, 1H), 7.95 (m, 1H), 7.88 (d, 1H), 7.67 (m, 1H), 7.53 (dd, 1H), 7.49 (dd, 1H), 7.40 (s, 1H), 7.24 (d, 1H), 6.97 (m, 2H), 5.21 (s, 2H), 4.58 (m, 2H), 4.33 (m, 2H); MS (EI) for C₂₅H₂₀N₄OS: 425 (MH⁺).

STEP 2: A suspension of 5-(4-quinolin-4-yl-2,3,4,5-tetrahydro-1,4-benzoxazepin-7-yl)-1,3-dihydro-2H-benzimidazole-2-thione (355 mg, 0.84 mmol), potassium carbonate (578 mg, 4.18 mmol), and methyl iodide (119 mg, 0.84 mmol) in dimethylformamide (5 mL) was stirred at room temperature for 1 h. Ethyl acetate (100 mL) was added, and the organic layer was washed with water (2×20 mL), 5% aqueous lithium chloride (2×20 mL), and brine (20 mL), dried over sodium sulfate, filtered and concentrated. Column chromatography on silica (dichloromethane/methanol 95:5) provided the title Compound (220 mg, 60% yield) as a brown solid. ¹H NMR (400 MHz, CDCl₃): 8.71 (d, 1H), 8.09 (m, 2H), 7.66 (m, 1H), 7.57-7.42 (m, 5H), 7.17 (d, 1H), 6.98 (d, 1H), 4.54 (s, 2H), 4.36 (m, 2H), 3.84 (m, 2H), 2.82 (s, 3H); MS (EI) for C₂₆H₂₂N₄OS: 439 (MH⁺).

### Example 20

### N-ethyl-6-(4-quinolin-4-yl-2,3,4,5-tetrahydro-1,4-benzoxazepin-7-yl)-1H-benzimidazol-2-amine

A solution of 7-[2-(methylthio)-1H-benzimidazol-6-yl]-4-quinolin-4-yl-2,3,4,5-tetrahydro-1,4-benzoxazepine (52 mg, 0.12 mmol, prepared according to the method of example 19) and ethylamine (1.5 mL) in ethanol (3 mL) heated with microwave irradiation at 150° C. for 8 h. Purification of the crude material by preparative reverse phase HPLC (0.1% aqueous ammonium acetate-acetonitrile) provided the title Compound as acetate salt (4 mg, 7% yield) as a colorless solid. ¹H NMR (400 MHz, methanol-d₄): 8.55 (d, 1H), 8.15 (d, 1H), 7.95 (d, 1H), 7.73 (m, 1H), 7.60-7.50 (m, 3H), 7.42 (m, 1H), 7.33 (dd, 1H), 7.29 (d, 1H), 7.09 (m, 2H), 4.69 (s, 2H), 4.41 (m, 2H), 3.92 (m, 2H), 3.44 (q, 2H), 1.94 (s, 3H), 1.31 (t, 3H); MS (EI) for C₂₇H₂₅N₅O: 436 (MH⁺).

Using analogous synthetic techniques and substituting with alternative starting reagents the following compounds of the invention were prepared. Alternative starting materials were obtained commercially unless otherwise indicated.

N-(1-methylethyl)-6-(4-quinolin-4-yl-2,3,4,5-tetrahydro-1,4-benzoxazepin-7-yl)-1H-benzimidazol-2-amine. Prepared as trifluoroacetate salt according to the method of example 20 by using isopropylamine. ¹H NMR (400 MHz, methanol-d₄): 8.38 (d, 1H), 8.33 (d, 1H), 7.98 (m, 1H), 7.91 (d, 1H), 7.76 (m, 1H), 7.68 (m, 1H), 7.60 (m, 2H), 7.56 (dd, 1H), 7.46 (d, 1H), 7.06 (m, 2H), 5.26 (s, 2H), 4.63 (m, 2H), 4.43 (m, 2H), 3.92 (h, 1H), 1.40 (d, 6H); MS (EI) for C₂₈H₂₇N₅O: 450 (MH⁺).

### Example 21

### Methyl (6-{4-[6,7-bis(methyloxy)quinolin-4-yl]-2,3,4,5-tetrahydro-1,4-benzoxazepin-7-yl}-1H-imidazo[4,5-b]pyridin-2-yl)carbamate

STEP 1: A mixture of commercially available 4-chloro-6,7-dimethoxyquinoline (120 mg, 0.53 mmol), 7-bromo-2,3-dihydro-1,4-benzoxazepine hydrochloride (example 2, step 1) (200 mg, 0.53 mmol), diisopropylethylamine (0.14 g, 1.1 mmol), in NMP (2 mL) was stirred in a microwave reactor at 120° C. for 45 min. After cooling to room temperature, the reaction mixture was purified directly by silica gel flash chromatography (0-10% methanol-dichloromethane gradient) to give 4-[6,7-bis(methyloxy)quinolin-4-yl]-7-bromo-2,3,4,5-tetrahydro-1,4-benzoxazepine (155 mg, 70% yield), MS (EI) for C₂₀H₁₉N₂O₃: 415 (MH⁺).

STEP 2: A mixture of commercially available 3-nitro-5-(4,4,5,5-tetramethyl-1,3,2-dioxaborolan-2-yl)pyridin-2-amine (125 mg, 0.47 mmol), 4-[6,7-bis(methyloxy)quinolin-4-yl]-7-bromo-2,3,4,5-tetrahydro-1,4-benzoxazepine (155 mg, 0.47 mmol), [1,1′-bis(diphenyl phosphino)ferrocene]dichloropalladium(II), complex with dichloromethane (34 mg, 0.05 mmol), cesium carbonate (300 mg, 2.4 mmol) in 1,4-dioxane (20 mL) and water (2 mL) was degassed with nitrogen for 5 minutes and then stirred at 93° C. for 18 hours. The reaction mixture was cooled to room temperature, diluted with ethyl acetate (80 mL) then filtered through a celite bed. The filtrate was washed with brine (2×50 mL), dried over sodium sulfate, filtered and concentrated. The residue was purified by silica gel flash chromatography (0 to 10% methanol-dichloromethane) to give 5-{4-[6,7-bis(methyloxy)quinolin-4-yl]-2,3,4,5-tetrahydro-1,4-benzoxazepin-7-yl}-3-nitropyridin-2-amine (135 mg, 76% yield); MS (EI) for C₂₅H₂₃N₅O₅: 474 (MH⁺).

STEP 3: A mixture of 5-{4-[6,7-bis(methyloxy)quinolin-4-yl]-2,3,4,5-tetrahydro-1,4-benzoxazepin-7-yl}-3-nitropyridin-2-amine (135 mg, 0.28 mmol), palladium (10% on charcoal, 135 mg) and methanol (15 mL) was hydrogenated in a Parr apparatus at 45 psi for 18 hours. The mixture was filtered then concentrated to give 5-{4-[6,7-bis(methyloxy)quinolin-4-yl]-2,3,4,5-tetrahydro-1,4-benzoxazepin-7-yl}pyridine-2,3-diamine (115 mg, 91% yield), MS (EI) for C₂₅H₂₅N₅O₃: 444 (MH⁺).

STEP 4: To a solution of 5-{4-[6,7-bis(methyloxy)quinolin-4-yl]-2,3,4,5-tetrahydro-1,4-benzoxazepin-7-yl}pyridine-2,3-diamine (85 mg, 0.19 mmol) in acetic acid (3 mL) was added 1,3-bis(methoxycarbonyl)-2-methyl-2-thiopseudourea (40 mg, 0.19 mmol). The reaction mixture was heated (65° C.) for 18 h and then concentrated. The resulting residue was dissolved in acetonitrile and purified by preparative reverse phase HPLC to provide methyl (6-{4-[6,7-bis(methyloxy)quinolin-4-yl]-2,3,4,5-tetrahydro-1,4-benzoxazepin-7-yl}-1H-imidazo[4,5-b]pyridin-2-yl)carbamate (55 mg, 54% yield) as a white solid. ¹H NMR (400 MHz, DMSO-D₆): δ 7.93 (br, 2H), 7.93 (br, 1H), 7.78 (br, 1H), 7.59 (dd, 1 h), 7.31 (s, 1H), 7.14 (dd, 1H), 7.09 (s, 1H), 6.95 (d, 1H), 4.60 (s, 2H), 4.43 (m, 2H), 3.89 (s, 3H), 3.78 (s, 3H), 3.73 (m, 2H), 3.53 (s, 3H), MS (EI) for C₂₈H₂₆N₆O₅: 527 (MH⁺).

### Example 22

### 4-(7-ethyl-5,6,7,8-tetrahydropyrido[3,4-d]pyrimidin-4-yl)-7-(2-methyl-1H-benzimidazol-6-yl)-2,3,4,5-tetrahydro-1,4-benzoxazepine

STEP 1: A solution of 7-(2-methyl-1H-benzimidazol-6-yl)-4-[7-(phenylmethyl)-5,6,7,8-tetrahydropyrido[3,4-d]pyrimidin-4-yl]-2,3,4,5-tetrahydro-1,4-benzoxazepine (Example 1) (0.13 g, 0.26 mmol) in a mixture of 50% acetic acid in methanol (5 mL) was hydrogenated in the presence of 10% Pd/C at 30 psi using a Parr shaker apparatus. The catalyst was filtered off and the solvent was concentrated to give 7-(2-methyl-1H-benzimidazol-6-yl)-4-(5,6,7,8-tetrahydropyrido[4,3-d]pyrimidin-4-yl)-2,3,4,5-tetrahydro-1,4-benzoxazepine (0.1 g, quantitative). MS (EI) for C₂₄H₂₄N₆O: 413 (MH⁺).

STEP 2: To a solution of 7-(2-methyl-1H-benzimidazol-6-yl)-4-(5,6,7,8-tetrahydropyrido[4,3-d]pyrimidin-4-yl)-2,3,4,5-tetrahydro-1,4-benzoxazepine (0.1 g, 0.26 mmol) and acetaldehyde (18 μL, 0.31 mmol) in a mixture of 10% aqueous tetrahydrofuran (5 mL) at 0° C. was added sodium triacetoxyborohydride (66 mg, 0.31 mmol) and the reaction mixture was stirred overnight at room temperature. The reaction mixture was diluted with ethyl acetate (50 mL), washed with brine (25 mL), dried over sodium sulfate, filtered and the solvent was concentrated. Purification by preparative reverse phase HPLC (0.1% aqueous ammonium acetate-acetonitrile) provided 4-(7-ethyl-5,6,7,8-tetrahydropyrido[3,4-d]pyrimidin-4-yl)-7-(2-methyl-1H-benzimidazol-6-yl)-2,3,4,5-tetrahydro-1,4-benzoxazepine (48 mg, 42%). ¹H NMR (400 MHz, d₆-DMSO): 8.44 (s, 1H), 7.94 (d, 1H), 7.83 (d, 1H), 7.76 (dd, 1H), 7.70 (d, 1H), 7.56 (dd, 1H), 7.06 (d, 1H), 4.84 (br d, 2H), 4.40 (br s, 4H), 4.18 (br s, 1H), 3.98 (br s, 1H), 3.62 (br s, 1H), 3.28 (dd, 2H), 3.18 (br s, 2H), 2.84 (br s, 1H), 1.22 (t, 3H). MS (EI) for C₂₆H₂₈N₆O: 441 (MH⁺).

### Example 23

### 6-(4-quinolin-4-yl-2,3,4,5-tetrahydro-1,4-benzoxazepin-7-yl)-1H-benzimidazol-2-amine

To a solution of 4-(4-quinolin-4-yl-2,3,4,5-tetrahydro-1,4-benzoxazepin-7-yl)-benzene-1,2-diamine (90 mg, 0.14 mmol, synthesized according to the method of example 2) in methanol (1 mL) was added a 3M solution of cyanogen bromide in dichloromethane (0.10 mL), and the reaction mixture was stirred at room temperature for 8 d. During this time additional cyanogen bromide was added after 1 d (0.10 mL), 2 d (0.20 mL), 3 d (0.20 mL), and 4 d (0.20 mL) reaction time. Ethyl acetate (50 mL) was added, and the solution was washed with 0.5N aqueous sodium hydroxide (2×50 mL), water (50 mL), and brine (50 mL), dried over sodium sulfate, filtered and concentrated. Purification by preparative reverse phase HPLC (0.1% aqueous ammonium acetate-acetonitrile) provided the title Compound as the acetate salt (8 mg, 7% yield) as an off-white solid. ¹H NMR (400 MHz, methanol-d₄): 8.54 (d, 1H), 8.14 (d, 1H), 7.94 (d, 1H), 7.73 (m, 1H), 7.60-7.47 (m, 4H), 7.38 (m, 1H), 7.31 (d, 1H), 7.10 (d, 1H), 7.07 (d, 1H), 4.70 (s, 2H), 4.41 (m, 2H), 3.93 (m, 2H), 1.94 (s, 3H); MS (EI) for C₂₅H₂₁N₅O: 408 (MH⁺).

### Example 24

### N-ethyl-6-[4-(2-methylquinazolin-4-yl)-2,3,4,5-tetrahydro-1,4-benzoxazepin-7-yl]-1H-benzimidazol-2-amine

STEP 1: To a solution of 1,1-dimethylethyl 7-(4-amino-3-nitrophenyl)-2,3-dihydro-1,4-benzoxazepine-4(5H)-carboxylate (10 g, 26 mmol) in dioxane (50 mL) was added hydrogen chloride in dioxane (4 M, 50 mL, 200 mmol), and the mixture was stirred at rt for 16 h. The volatile materials were then removed to provide 2-nitro-4-(2,3,4,5-tetrahydro-1,4-benzoxazepin-7-yl)aniline dihydrochloride in quantitative yield. MS (EI) for C₁₅H₁₅N₃O₃: 286 (MH⁺).

STEP 2: A solution of 2-nitro-4-(2,3,4,5-tetrahydro-1,4-benzoxazepin-7-yl)aniline dihydrochloride (323 mg, 1.0 mmol), 4-chloro-2-methylquinazoline (179 mg, 1.0 mmol), and diisopropylethylamine (700 uL, 4.0 mmol) in NMP (II mL) was heated to 90° C. and stirred for 40 min. After cooling to rt, water was added to the reaction mixture. The orange precipitate formed was collected by filtration and then dried to provide 4-[4-(2-methylquinazolin-4-yl)-2,3,4,5-tetrahydro-1,4-benzoxazepin-7-yl]-2-nitroaniline (380 mg, 0.89 mmol, 89% yield) as a bright orange powder. MS (EI) for C₂₄H₂₁N₅O₃: 428 (MH⁺).

STEP 3: To a solution of 4-[4-(2-methylquinazolin-4-yl)-2,3,4,5-tetrahydro-1,4-benzoxazepin-7-yl]-2-nitroaniline (380 mg, 0.89 mmol) in THF (12 mL) was added palladium on carbon (wet, 100 mg). The resulting suspension was subjected to an atmosphere of hydrogen at 40 psi for 5 h. The catalyst was removed by filtration through celite, and the filtrate was concentrated to provide 4-[4-(2-methylquinazolin-4-yl)-2,3,4,5-tetrahydro-1,4-benzoxazepin-7-yl]benzene-1,2-diamine (333 mg, 0.84 mmol, 94% yield) as a yellow-orange solid. MS (EI) for C₂₄H₂₃N₅O: 398 (MH⁺).

STEP 4: To a solution of 4-[4-(2-methylquinazolin-4-yl)-2,3,4,5-tetrahydro-1,4-benzoxazepin-7-yl]benzene-1,2-diamine (333 mg, 0.84 mmol) in THF (5 mL) at 0° C. was added ethyl isothiocyanate (74 uL, 0.84 mmol). After stirring for 2 h at 0° C., the reaction mixture was warmed to rt for 1 h and was then heated to 45° C. for 4 h. The mixture was then cooled back to rt and allowed to stir for 3 d. Addition of water was followed by extraction into ethyl acetate. The organic phase was then dried over magnesium sulfate, filtered, and concentrated. The residue obtained was then dissolved in acetonitrile (5 mL) and ethyl acetate (2 mL). To this solution was added N-(3-dimethylaminopropyl)-N′-ethylcarbodiimide hydrochloride (161 mg, 0.84 mmol). The mixture was heated to reflux for 1.5 h and was then cooled to rt. Water and ethyl acetate were then added. The biphasic mixture was filtered to remove solid materials, and the filtrate was then partitioned. The aqueous phase was extracted with ethyl acetate. The combined organic extracts were dried over magnesium sulfate, filtered, and then concentrated. The residue was purified by reverse-phase preparative HPLC to provide N-ethyl-6-[4-(2-methylquinazolin-4-yl)-2,3,4,5-tetrahydro-1,4-benzoxazepin-7-yl]-1H-benzimidazol-2-amine as a diacetate salt (137 mg, 0.24 mmol, 29% yield). ¹H NMR (400 MHz, dmso) δ 8.01 (d, 1H), 7.79-7.72 (m, 1H), 7.72-7.67 (m, 1H), 7.60 (d, 1H), 7.47-7.39 (m, 2H), 7.35 (s, 1H), 7.20-7.11 (m, 2H), 6.99 (d, 1H), 6.72 (br s, 1H), 5.02 (s, 2H), 4.47-4.38 (m, 2H), 4.22-4.13 (m, 2H), 3.37-3.27 (m, 2H), 2.48 (s, 3H), 1.89 (s, 7H), 1.18 (t, 3H); MS (EI) for C₂₇H₂₆N₆O: 451 (MH⁺).

Using analogous synthetic techniques and substituting with alternative starting reagents in step 4 the following compounds of the invention were prepared. Alternative starting materials were obtained commercially unless otherwise indicated.

N-ethyl-6-[4-(7-fluoroquinolin-4-yl)-2,3,4,5-tetrahydro-1,4-benzoxazepin-7-yl]-1H-benzimidazol-2-amine. Prepared according to the method of example 24 by using 4-chloro-7-fluoroquinoline in step 2. ¹H NMR (400 MHz, CD₃OD): 8.50 (d, 1H), 8.09 (t, 1H), 7.56-7.44 (m, 4H), 7.38-7.26 (m, 3H), 7.06 (d, 1H), 6.97 (d, 1H), 4.67 (s, br, 2H), 4.36 (s, br, 2H), 3.85 (s, br, 2H), 3.46 (q, 2H), 1.37 (t, 3H); MS (EI) for C₂₇H₂₄FN₅O: 454 (MH⁺).

N-ethyl-6-[4-(8-fluoroquinolin-4-yl)-2,3,4,5-tetrahydro-1,4-benzoxazepin-7-yl]-1H-benzimidazol-2-amine. Prepared according to the method of example 24 by using 4-chloro-8-fluoroquinoline in step 2. ¹H NMR (400 MHz, CD₃OD): 8.53 (d, 1H), 7.85 (d, 1H), 7.52-7.36 (m, 5H), 7.22 (m, 2H), 7.07-7.02 (m, 2H), 4.56 (s, 2H), 4.36-4.30 (m, 2H), 3.84-3.78 (m, 2H), 3.42 (q, 2H), 1.28 (t, 3H); MS (EI) for C₂₇H₂₅FN₅O₂: 452 (MH⁺).

N-ethyl-6-[4-(6-fluoroquinolin-4-yl)-2,3,4,5-tetrahydro-1,4-benzoxazepin-7-yl]-1H-benzimidazol-2-amine. Prepared according to the method of example 24 by using reagent 4-chloro-6-fluoroquinoline in step 2. ¹H NMR (400 MHz, CD₃OD): 8.55 (d, 1H), 8.02-7.96 (dd, 1H), 7.79-7.73 (dd, 1H), 7.60-7.42 (m, 4H), 7.30-7.21 (m, 2H), 7.12-7.07 (m, 2H), 4.56 (s, 2H), 4.39-4.34 (m, 2H), 3.84-3.79 (m, 2H), 3.42 (q, 2H), 1.29 (t, 3H); MS (EI) for C₂₇H₂₄FN₅O: 454 (MH⁺).

### Example 25

### N-ethyl-5-methyl-6-[7-(2-methyl-1H-imidazo[4,5-b]pyridin-6-yl)-2,3-dihydro-1,4-benzoxazepin-4(5H)-yl]pyrimidin-4-amine and 4-(6-chloro-5-methylpyrimidin-4-yl)-7-(2-methyl-1H-imidazo[4,5-b]pyridin-6-yl)-2,3,4,5-tetrahydro-1,4-benzoxazepine

STEP 1: A solution of 1-methylpropyl 2-methyl-6-(2,3,4,5-tetrahydro-1,4-benzoxazepin-7-yl)-1H-imidazo[4,5-b]pyridine-1-carboxylate hydrochloride (0.213 g, 0.512 mmol, example 6, step 2), 4,6-dichloro-5-methylpyrimidine (0.100 g, 0.613 mmol), and diisopropylethylamine (0.330 g, 2.56 mmol) in N-methylpyrrolidinone (2 mL) was stirred at room temperature for 17 h. The reaction mixture was diluted with ethyl acetate (100 mL), washed with saturated sodium bicarbonate (50 mL) and brine (25 mL), and dried over sodium sulfate. Filtration, concentration and purification by column chromatography on silica (97:3 dichloromethane/methanol) provided 4-(6-chloro-5-methylpyrimidin-4-yl)-7-(2-methyl-1H-imidazo[4,5-b]pyridin-6-yl)-2,3,4,5-tetrahydro-1,4-benzoxazepine (0.06 g, 31% yield) as a white solid. ¹H NMR (400 MHz, CD₃OD) δ 8.50 (s, 1H), 8.22 (s, 1H), 8.04 (s, 1H), 7.60 (s, 1H), 7.50 (d, 1H), 7.08 (d, 1H), 4.81 (s, 2H), 4.39-4.32 (m, 2H), 4.03-3.97 (m, 2H), 2.64 (s, 3H), 2.37 (s, 3H); MS (EI) for C₂₁H₁₉ClN₆O: 407 (MH⁺).

STEP 2: A suspension of 4-(6-chloro-5-methylpyrimidin-4-yl)-7-(2-methyl-1H-imidazo[4,5-b]pyridin-6-yl)-2,3,4,5-tetrahydro-1,4-benzoxazepine (44 mg, 0.11 mmol), and ethylamine (29 mg, 0.65 mmol), in N-methylpyrrolidinone (2 mL) was stirred at 100° C. for 6 h. The reaction mixture was diluted with ethyl acetate (75 mL), washed with saturated sodium bicarbonate (75 mL) and brine (50 mL), and dried over sodium sulfate. Concentration and purification by preparatory HPLC (0.1% aqueous ammonium acetate-acetonitrile) gave N-ethyl-5-methyl-6-[7-(2-methyl-1H-imidazo[4,5-b]pyridin-6-yl)-2,3-dihydro-1,4-benzoxazepin-4(5H)-yl]pyrimidin-4-amine (13 mg, 30% yield) as a white solid. ¹H NMR (400 MHz, CD₃OD) δ 8.50 (s, 1H), 8.07 (s, 1H), 8.04 (d, 1H), 7.56-7.48 (m, 2H), 7.11 (d, 1H), 4.57 (s, 2H), 4.33 (d, 2H), 3.79 (d, 2H), 3.53-3.39 (q, 2H), 2.64 (s, 3H), 2.04 (s, 3H), 1.21 (t, 3H); MS (EI) for C₂₃H₂₅N₇O: 416 (MH⁺).

Using analogous synthetic techniques and substituting with alternative starting reagents the following compounds of the invention were prepared. Alternative starting materials were obtained commercially unless otherwise indicated.

N,5-dimethyl-6-[7-(2-methyl-1H-imidazo[4,5-b]pyridin-6-yl)-2,3-dihydro-1,4-benzoxazepin-4(5H)-yl]pyrimidin-4-amine. Prepared as diacetate salt according to the method of example 25 by using methylamine in step 2. ¹H NMR (400 MHz, methanol-d₄): 8.49 (s, 1H), 8.07 (s, 1H), 8.03 (m, 1H), 7.50 (m, 2H), 7.11 (d, 1H), 4.49 (s, 2H), 4.30 (m, 2H), 3.74 (m, 2H), 2.93 (s, 3H), 2.64 (s, 3H), 2.02 (s, 3H), 1.93 (s, 6H); MS (EI) for C₂₂H₂N₇O: 402 (MH⁺).

5-methyl-N-(1-methylethyl)-6-[7-(2-methyl-1H-imidazo[4,5-b]pyridin-6-yl)-2,3-dihydro-1,4-benzoxazepin-4(5H)-yl]pyrimidin-4-amine. Prepared according to the method of example 25 by using isopropylamine in step 2. ¹H NMR (400 MHz, methanol-d₄): 8.50 (s, 1H), 8.05 (s, 2H), 7.51 (m, 2H), 7.12 (d, 1H), 4.49 (s, 2H), 4.31 (m, 2H), 4.26 (m, 1H), 3.74 (m, 2H), 2.64 (s, 3H), 2.02 (s, 3H), 1.22 (d, 6H); MS (EI) for C₂₄H₂₇N₇O: 430 (MH⁺).

7-(2-methyl-1H-imidazo[4,5-b]pyridin-6-yl)-4-(5-methyl-6-morpholin-4-ylpyrimidin-4-yl)-2,3,4,5-tetrahydro-1,4-benzoxazepine. Prepared according to the method of example 25 by using morpholine in step 2. ¹H NMR (400 MHz, methanol-d₄): 8.49 (s, 1H), 8.11 (s, 1H), 8.03 (s, 1H), 7.57 (d, 1H), 7.48 (dd, 1H), 7.09 (d, 1H), 4.71 (s, 2H), 4.35 (m, 2H), 3.92 (m, 2H), 3.75 (t, 4H), 3.33 (t, 4H), 2.64 (s, 3H), 2.19 (s, 3H); MS (EI) for C₂₅H₂₇N₇O₂: 458 (MH⁺).

7-(2-methyl-1H-imidazo[4,5-b]pyridin-6-yl)-4-[5-methyl-6-(4-methylpiperazin-1-yl)pyrimidin-4-yl]-2,3,4,5-tetrahydro-1,4-benzoxazepine. Prepared as triacetate salt according to the method of example 25 by using N-methylpiperazine in step 2. ¹H NMR (400 MHz, methanol-d₄): 8.50 (s, 1H), 8.10 (s, 1H), 8.04 (s, 1H), 7.56 (d, 1H), 7.49 (dd, 1H), 7.09 (d, 1H), 4.71 (s, 2H), 4.34 (m, 2H), 3.92 (m, 2H), 3.40 (m, 4H), 2.64 (s, 3H), 2.59 (m, 4H), 2.34 (s, 3H), 2.19 (s, 3H), 1.93 (s, 9H); MS (EI) for C₂₆H₃₀N₈O: 471 (MH⁺).

4-(6-azetidin-1-yl-5-methylpyrimidin-4-yl)-7-(2-methyl-1H-imidazo[4,5-b]pyridin-6-yl)-2,3,4,5-tetrahydro-1,4-benzoxazepine. Prepared according to the method of example 25 by using azetidine in step 2. ¹H NMR (400 MHz, methanol-d₄): 8.50 (s, 1H), 8.04 (s, 1H), 7.99 (s, 1H), 7.55 (d, 1H), 7.49 (dd, 1H), 7.09 (d, 1H), 4.62 (s, 2H), 4.32 (m, 2H), 4.18 (t, 4H), 3.84 (m, 2H), 2.63 (s, 3H), 2.33 (m, 2H), 2.07 (s, 3H); MS (EI) for C₂₄H₂₅N₇O: 428 (MH⁺).

N-{6-[7-(2-cyclopropyl-1H-imidazo[4,5-b]pyridin-6-yl)-2,3-dihydro-1,4-benzoxazepin-4(5H)-yl]-5-methylpyrimidin-4-yl}-N,N′-dimethylethane-1,2-diamine. Prepared as acetate salt according to the method of example 25 by using 1-methylpropyl 2-cyclopropyl-6-(2,3,4,5-tetrahydro-1,4-benzoxazepin-7-yl)-1H-imidazo[4,5-b]pyridine-1-carboxylate hydrochloride (example 6) in step 1 and N,N′-dimethylethylenediamine in step 2. ¹H NMR (400 MHz, methanol-d₄): 8.44 (d, 1H), 8.06 (s, 1H), 7.96 (d, 1H), 7.55 (d, 1H), 7.46 (dd, 1H), 7.06 (d, 1H), 4.75 (s, 2H), 4.34 (m, 2H), 3.94 (m, 2H), 3.69 (t, 2H), 3.21 (t, 2H), 3.07 (s, 3H), 2.67 (s, 3H), 2.21 (m, 4H), 1.91 (s, 3H), 1.22 (m, 4H); MS (EI) for C₂₇H₃₂N₈O: 485 (MH⁺).

6-[7-(2-cyclopropyl-1H-imidazo[4,5-b]pyridin-6-yl)-2,3-dihydro-1,4-benzoxazepin-4(5H)-yl]-5-methyl-N-(1-methylpiperidin-4-yl)pyrimidin-4-amine. Prepared according to the method of example 25 by using 1-methylpropyl 2-cyclopropyl-6-(2,3,4,5-tetrahydro-1,4-benzoxazepin-7-yl)-1H-imidazo[4,5-b]pyridine-1-carboxylate hydrochloride (example 6) in step 1 and 4-amino-1-methylpiperidine in step 2. ¹H NMR (400 MHz, methanol-d₄): 8.46 (s, 1H), 8.06 (s, 1H), 7.98 (s, 1H), 7.50 (m, 2H), 7.12 (d, 1H), 4.52 (s, 2H), 4.32 (m, 2H), 4.04 (m, 1H), 3.77 (m, 2H), 3.13 (m, 2H), 2.56 (m, 2H), 2.53 (s, 3H), 2.21 (m, 1H), 2.08 (m, 2H), 2.05 (s, 3H), 1.71 (m, 2H), 1.23 (m, 4H); MS (EI) for C₂₉H₃₄N₈O: 511 (MH⁺).

6-[7-(2-cyclopropyl-1H-imidazo[4,5-b]pyridin-6-yl)-2,3-dihydro-1,4-benzoxazepin-4(5H)-yl]-N,5-dimethyl-N-[(1R)-1-phenylethyl]pyrimidin-4-amine. Prepared according to the method of example 25 by using 1-methylpropyl 2-cyclopropyl-6-(2,3,4,5-tetrahydro-1,4-benzoxazepin-7-yl)-1H-imidazo[4,5-b]pyridine-1-carboxylate hydrochloride (example 6) in step 1 and (R)-(+)-N-methyl-1-phenylethylamine in step 2. ¹H NMR (400 MHz, methanol-d₄): 8.43 (s, 1H), 8.02 (s, 1H), 7.92 (s, 1H), 7.54 (d, 1H), 7.48 (dd, 1H), 7.17-7.08 (m, 6H), 5.28 (m, 1H), 4.72 (m, 2H), 4.36 (m, 2H), 3.94 (m, 2H), 2.63 (s, 3H), 2.25 (s, 3H), 2.19 (m, 1H), 1.59 (d, 3H), 1.21 (m, 4H); MS (EI) for C₃₂H₃₃N₇O: 532 (MH⁺).

6-[7-(2-cyclopropyl-1H-imidazo[4,5-b]pyridin-6-yl)-2,3-dihydro-1,4-benzoxazepin-4(5H)-yl]-N,5-dimethyl-N-[(1S)-1-phenylethyl]pyrimidin-4-amine. Prepared according to the method of example 25 by using 1-methylpropyl 2-cyclopropyl-6-(2,3,4,5-tetrahydro-1,4-benzoxazepin-7-yl)-1H-imidazo[4,5-b]pyridine-1-carboxylate hydrochloride (example 6) in step 1 and (S)-(−)-N-methyl-1-phenylethylamine in step 2. ¹H NMR (400 MHz, methanol-d₄): 8.43 (s, 1H), 8.02 (s, 1H), 7.92 (s, 1H), 7.54 (d, 1H), 7.48 (dd, 1H), 7.17-7.08 (m, 6H), 5.28 (m, 1H), 4.72 (m, 2H), 4.36 (m, 2H), 3.94 (m, 2H), 2.63 (s, 3H), 2.25 (s, 3H), 2.19 (m, 1H), 1.59 (d, 3H), 1.21 (m, 4H); MS (EI) for C₃₂H₃₃N₇O: 532 (MH⁺).

6-[7-(2-cyclopropyl-1H-imidazo[4,5-b]pyridin-6-yl)-2,3-dihydro-1,4-benzoxazepin-4(5H)-yl]-5-methyl-N-[(1-methylpiperidin-4-yl)methyl]pyrimidin-4-amine. Prepared according to the method of example 25 by using 1-methylpropyl 2-cyclopropyl-6-(2,3,4,5-tetrahydro-1,4-benzoxazepin-7-yl)-1H-imidazo[4,5-b]pyridine-1-carboxylate hydrochloride (example 6) in step 1 and 4-aminomethyl-1-methylpiperidine in step 2. ¹H NMR (400 MHz, methanol-d₄): 8.44 (d, 1H), 8.03 (s, 1H), 7.97 (d, 1H), 7.48 (m, 2H), 7.10 (d, 1H), 4.50 (m, 2H), 4.31 (m, 2H), 3.75 (m, 2H), 3.35 (m, 2H), 3.24 (m, 2H), 2.62 (s, 3H), 2.58 (m, 2H), 2.21 (m, 1H), 2.04 (s, 3H), 1.88 (m, 2H), 1.41 (m, 2H), 1.22 (m, 4H); MS (EI) for C₃₀H₃₆N₈O: 525 (MH⁺).

6-[7-(2-cyclopropyl-1H-imidazo[4,5-b]pyridin-6-yl)-2,3-dihydro-1,4-benzoxazepin-4(5H)-yl]-5-methyl-N-[2-(1-methylpyrrolidin-2-yl)ethyl]pyrimidin-4-amine. Prepared as acetate salt according to the method of example 25 by using 1-methylpropyl 2-cyclopropyl-6-(2,3,4,5-tetrahydro-1,4-benzoxazepin-7-yl)-1H-imidazo[4,5-b]pyridine-1-carboxylate hydrochloride (example 6) in step 1 and 2-aminoethyl-1-methylpyrrolidine in step 2. ¹H NMR (400 MHz, methanol-d₄): 8.44 (d, 1H), 8.07 (s, 1H), 7.97 (d, 1H), 7.48 (m, 2H), 7.10 (d, 1H), 4.52 (m, 2H), 4.31 (m, 2H), 3.76 (m, 2H), 3.52 (m, 2H), 3.46 (m, 1H), 3.08 (m, 1H), 2.91 (m, 1H), 2.74 (s, 3H), 2.31 (m, 1H), 2.19 (m, 2H), 2.04 (s, 3H), 1.98 (m, 1H), 1.92 (s, 3H), 1.75 (m, 2H), 1.22 (m, 4H); MS (EI) for C₃₀H₃₆N₈O: 525 (MH⁺).

6-[7-(2-cyclopropyl-1H-imidazo[4,5-b]pyridin-6-yl)-2,3-dihydro-1,4-benzoxazepin-4(5H)-yl]-5-methyl-N-(tetrahydro-2H-pyran-4-yl)pyrimidin-4-amine. Prepared according to the method of example 25 by using 1-methylpropyl 2-cyclopropyl-6-(2,3,4,5-tetrahydro-1,4-benzoxazepin-7-yl)-1H-imidazo[4,5-b]pyridine-1-carboxylate hydrochloride (example 6) in step 1 and 4-aminotetrahydropyran in step 2. ¹H NMR (400 MHz, methanol-d₄): 8.47 (s, 1H), 8.11 (s, 1H), 7.99 (s, 1H), 7.52 (m, 2H), 7.11 (d, 1H), 4.66 (s, 2H), 4.35 (m, 2H), 4.11 (m, 1H), 3.98 (m, 2H), 3.84 (m, 2H), 3.50 (m, 2H), 2.22 (m, 1H), 2.07 (s, 3H), 1.89 (m, 2H), 1.67 (m, 2H), 1.23 (m, 4H); MS (EI) for C₂₈H₃₁N₇O₂: 498 (MH⁺).

N-ethyl-2,5-dimethyl-6-[7-(2-methyl-1H-imidazo[4,5-b]pyridin-6-yl)-2,3-dihydro-1,4-benzoxazepin-4(5H)-yl]pyrimidin-4-amine. Prepared according to the method of example 25 by using 4,6-dichloro-2,5-dimethylpyrimidine in step 1. ¹H NMR (400 MHz, methanol-d₄): 8.49 (d, 1H), 8.03 (d, 1H), 7.49 (m, 2H), 7.11 (d, 1H), 4.46 (s, 2H), 4.27 (m, 2H), 3.72 (m, 2H), 3.45 (q, 2H), 2.63 (s, 3H), 2.34 (s, 3H), 1.98 (s, 3H), 1.19 (t, 3H); MS (EI) for C₂₄H₂₇N₇O: 430 (MH⁺).

4-{7-[2-(ethylamino)-1H-benzimidazol-6-yl]-2,3-dihydro-1,4-benzoxazepin-4(5H)-yl}-N-methylquinazolin-2-amine. Prepared according to the method of example 25 by using N-ethyl-5-(2,3,4,5-tetrahydro-1,4-benzoxazepin-7-yl)-1H-benzimidazol-2-amine dihydrochloride (example 11, step 3) and 2,4-dichloroquinazoline in step 1 and methylamine in step 2. ¹H NMR (400 MHz, DMSO-d6) δ 7.80 (d, 1H), 7.51 (d, 2H), 7.43 (d, 1H), 7.33 (s, 1H), 7.15 (t, 2H), 7.06-6.96 (m, 2H), 6.74-6.61 (m, 2H), 4.94 (s, 2H), 4.43 (s, 2H), 4.07 (s, 2H), 3.31 (q, 2H), 2.77 (s, 3H), 1.87 (s, 6H), 1.18 (t, 3H); MS (EI) for C₂₇H₂₇N₇O: 466.

N-ethyl-4-{7-[2-(ethylamino)-1H-benzimidazol-6-yl]-2,3-dihydro-1,4-benzoxazepin-4(5H)-yl}quinazolin-2-amine. Prepared according to the method of example 25 by using N-ethyl-5-(2,3,4,5-tetrahydro-1,4-benzoxazepin-7-yl)-1H-benzimidazol-2-amine dihydrochloride (example 11, step 3) and 2,4-dichloroquinazoline in step 1 and ethylamine in step 2. ¹H NMR (400 MHz, DMSO-d6) δ 7.80 (d, 1H), 7.57-7.48 (m, 2H), 7.43 (s, 1H), 7.36-7.28 (m, 2H), 7.19-7.11 (m, 2H), 7.07-6.95 (m, 2H), 6.72-6.58 (m, 1H), 4.95 (s, 2H), 4.43 (s, 2H), 4.11 (s, 2H), 3.32-3.25 (m, 4H), 1.91 (d, 4H), 1.18 (t, 6H); MS (EI) for C₂₈H₂₉N₇O: 480 (MH⁺).

N-ethyl-6-{4-[6-(ethylamino)-5-methylpyrimidin-4-yl]-2,3,4,5-tetrahydro-1,4-benzoxazepin-7-yl}-1H-benzimidazol-2-amine. Prepared as an acetate salt according to the method of example 25 by using N-ethyl-5-(2,3,4,5-tetrahydro-1,4-benzoxazepin-7-yl)-1H-benzimidazol-2-amine dihydrochloride (example 11, step 3) in step 1 and ethylamine in step 2. ¹H NMR (400 MHz, DMSO-d6) δ 8.01 (s, 1H), 7.45 (br s, 1H), 7.38 (d, 1H), 7.30 (br s, 1H), 7.16-7.04 (m, 2H), 6.98 (d, 1H), 6.60 (t, 1H), 6.42 (t, 1H), 4.40 (s, 2H), 4.24-4.18 (m, 2H), 3.68-3.61 (m, 2H), 3.30-3.25 (m, 4H), 1.96 (s, 3H), 1.89-1.86 (m, 3H), 1.16 (t, 3H), 1.08 (t, 3H); MS (EI) for C₂₅H₂₉N₇O: 444 (MH⁺).

### Example 26

### 6-[4-(6-methylquinazolin-4-yl)-2,3,4,5-tetrahydro-1,4-benzoxazepin-7-yl]-1H-imidazo[4,5-b]pyridin-2-amine

STEP 1: A suspension of 5-bromo-3-nitropyridin-2-amine (4.84 g, 22.2 mmol), (4-{[(1,1-dimethylethyl)oxy]carbonyl}-2,3,4,5-tetrahydro-1,4-benzoxazepin-7-yl)boronic acid (6.51 g, 22.2 mmol) (example 1, step 2), dichloro[1,1-bis(diphenyl)-phosphino]ferrocenepalladium (II) dichloromethane adduct (1.60 g, 10 mol %) in dioxane (75 mL) and water (15 mL) was degassed with nitrogen, and then cesium carbonate (14.46 g, 44.4 mmol) was added. The reaction mixture was stirred at 90° C. overnight. The mixture was cooled to room temperature, water (150 ml) was added and stirred for 30 min to give a precipitate. The product 1,1-dimethylethyl-7-(6-amino-5-nitropyridin-3-yl)-2,3-dihydro-1,4-benzoxazepine-4(5H)-carboxylate (8.1 g, 94% yield) was collected by filtration, dried under vacuum. MS (EI) for C₁₉H₂₂N₄O₅: 387.1 (MH⁺).

STEP 2: A suspension of 1,1-dimethylethyl-7-(6-amino-5-nitropyridin-3-yl)-2,3-dihydro-1,4-benzoxazepine-4(5H)-carboxylate (6.12 g, 15.84 mmol), palladium on carbon (0.6 g) in acetic acid (100 mL) was degassed with nitrogen for 10 minutes. The reaction mixture was hydrogenated (45 psi) on a parr shaker for 60 minutes. Upon completion of hydrogenation, the reaction mixture was filtered through a pad of celite. The filtrate was concentrated and the residue was diluted with ethyl acetate (200 ml), washed with water, saturated sodium bicarbonate solution and brine then dried over sodium sulfate and filtered. Evaporation of ethyl acetate afforded 1,1-dimethylethyl-7-(5,6-diaminopyridin-3-yl)-2,3-dihydro-1,4-benzoxazepine-4(5H)-carboxylate (5.6 g, 99% yield). ¹H NMR (400 MHz, CD₃OD): 7.56-7.27 (m, 4H), 7.08-7.02 (m, 1H), 4.51 (s, 2H), 4.07-4.03 (m, 2H), 3.86-3.74 (m, 2H), 1.3 (s, 9H).

STEP 3: A solution of 1,1-dimethylethyl-7-(5,6-diaminopyridin-3-yl)-2,3-dihydro-1,4-benzoxazepine-4(5H)-carboxylate (5.64 g, 15.84 mmol) and 1,3-bis(benzyloxycarbonyl)-2-methyl-2-thiopseudourea (11.8 g, 32.92 mmol) in acetic acid (30 mL) was heated to 86° C. for 3 hours. After cooling to room temperature, ethyl acetate (100 mL) was added and the precipitate was collected by filtration, washed several times with ethyl acetate, and dried to give 1,1-dimethylethyl-7-[2-({[(phenylmethyl)oxy]carbonyl}amino)-1H-imidazo[4,5-b]pyridine-6-yl]-2,3-dihydro-1,4-benzoxazepine-4(5H)-carboxylate (4.50 g, 55% yield). ¹HNMR (400 MHz, DMSO-d₆): 11.9 (s, br, 2H), 8.42 (s, 1H), 7.90 (s, 1H), 7.52-7.36 (m, 7H), 5.28 (s, 2H), 4.60-4.34 (m, 2H), 4.18-4.02 (m, 2H), 3.79-3.67 (m, 2H), 1.38 (s, 9H).

STEP 4: To the solution of 1,1-dimethylethyl-7-[2-({[(phenylmethyl)oxy]carbonyl}amino)-1H-imidazo[4,5-b]pyridine-6-yl]-2,3-dihydro-1,4-benzoxazepine-4(5H)-carboxylate (4.5 g, 8.73 mmol) in methanol (20 ml) was added 4 N HCl in dioxane at room temperature. Then the reaction mixture was heated to 55° C. for 3 hours. After cooling to room temperature, the precipitate collected by filtration, washed with a minimum of methanol and dried to give phenylmethyl[6-(2,3,4,5-tetrahydro-1,4-benzoxazepin-7-yl)-1H-imidazo[4,5-b]pyridin-2-yl]carbamate hydrochloride (3.0 g, 76% yield). ¹HNMR (400 MHz, CD₃OD): 8.59 (m, 1H), 8.51 (s, 1H), 7.85 (m, 1H), 7.79-7.75 (m, 1H), 5.37 (s, 2H), 4.53 (s, 2H), 4.37-4.32 (m, 2H), 3.67-3.64 (m, 2H).

STEP 5: Diisopropylethylamine (0.23 g, 1.76 mmol) was added to a solution of phenylmethyl[6-(2,3,4,5-tetrahydro-1,4-benzoxazepin-7-yl)-1H-imidazo[4,5-b]pyridin-2-yl]carbamate hydrochloride (0.2 g, 0.44 mmol) and 4-chloro-6-methylquinazoline (0.08 g, 0.44 mmol), in NMP (5 ml) at room temperature. The reaction mixture was heated to 90° C. for 30 minutes, and then cooled to room temperature. Water was added and the resulting suspension was stirred overnight. The precipitate was collected by filtration and dried under vacuum to give phenylmethyl{6-[4-(6-methylquinazolin-4-yl)-2,3,4,5-tetrahydro-1,4-benzoxazepin-7-yl]-3H-imidazo[4,5-b]pyridin-2-yl}carbamate (0.215 g, 87% yield). MS (EI) for C₃₂H₂₇N₇O₃: 558.1 (MH⁺).

STEP 6: Phenylmethyl{6-[4-(6-methylquinazolin-4-yl)-2,3,4,5-tetrahydro-1,4-benzoxazepin-7-yl]-3H-imidazo[4,5-b]pyridin-2-yl}carbamate (0.1 g, 0.18 mmol) in acetic acid (8 ml) was placed under nitrogen. Palladium on carbon (0.25 g, 10 W %) was added and the reaction mixture saturated with hydrogen then stirred at room temperature for 12 h. The reaction mixture was filtered through a pad of celite then concentrated. The residue was taken into a minimum of methanol and purified by preparative reverse phase HPLC to give 6-[4-(6-methylquinazolin-4-yl)-2,3,4,5-tetrahydro-1,4-benzoxazepin-7-yl]-3H-imidazo[4,5-b]pyridin-2-amine (0.0033 g). ¹H NMR (400 MHz, d₆-DMSO); δ 8.52 (s, 1H), 8.32-8.12 (s, 1H), 7.84-7.47 (m, 6H), 7.07-7.00 (d, 1H), 6.80-6.50 (s, 1H), 5.04 (s, 2H), 4.56-4.44 (m, 2H), 4.20-4.09 (m, 2H), 2.83 (s, 3H); MS (EI) for C₂₄H₂₁N₇O: 424.1 (MH⁺).

Using analogous synthetic techniques and substituting with alternative starting reagents in steps 1, 3 or 5 the following compounds of the invention were prepared. Alternative starting materials were obtained commercially unless otherwise indicated.

6-[7-(1H-benzimidazol-6-yl)-2,3-dihydro-1,4-benzoxazepin-4(5H)-yl]-2,5-dimethyl-N-phenylpyrimidin-4-amine. Synthesized according to the method of example 26 using 4-bromo-2-nitroaniline in step 1, triethyl orthoformate in refluxing ethanol step 3, and 6-chloro-2,5-dimethyl-N-phenylpyrimidin-4-amine (Prepared according to the general method in Journal of Medicinal Chemistry (1996), 39(22), 4358-4360) in step 5. ¹H NMR (400 MHz, dmso-d6) δ 12.03 (brs, 1H), 8.21 (s, 1H), 8.18 (s, 1H), 7.80 (brs, 1H), 7.60 (m, 6H), 7.21 (t, 2H), 7.18 (d, 1H), 6.92 (t, 1H), 4.60 (s, 2H), 4.23 (brs, 2H), 3.78 (brs, 2H), 2.23 (s, 3H), 2.18 (s, 3H); MS (EI) for C₂₈H₂₆N₆O: 463.2 (MH⁺).

6-[7-(1H-imidazo[4,5-b]pyridin-6-yl)-2,3-dihydro-1,4-benzoxazepin-4(5H)-yl]-2,5-dimethyl-N-phenylpyrimidin-4-amine. Synthesized according to the method of example 26 using triethyl orthoformate in refluxing ethanol step 3 and 6-chloro-2,5-dimethyl-N-phenylpyrimidin-4-amine (Prepared according to the general method in Journal of Medicinal Chemistry (1996), 39(22), 4358-4360) in step 5. ¹H NMR (400 MHz, MeOH-d4): δ 8.61 (s, 1H), 8.41 (s, 1H), 8.21 (s, 1H), 7.60 (m, 4H), 7.21 (t, 2H), 7.18 (d, 1H), 6.92 (t, 1H), 4.60 (s, 2H), 4.23 (brs, 2H), 3.78 (brs, 2H), 2.23 (s, 3H), 2.18 (s, 3H); MS (EI) for C₂₇H₂₅N₇O: 464.2 (MH⁺).

7-(1H-benzimidazol-6-yl)-4-pyrimidin-4-yl-2,3,4,5-tetrahydro-1,4-benzoxazepine. Synthesized according to the method of example 26 by using 4-bromo-2-nitroaniline in step 1, triethylorthoformate in refluxing ethanol step 3, and using 4-chloropyrimidine in step 5. ¹H NMR (400 MHz, DMSO-d6) δ 8.49 (s, 1H), 8.25 (s, 1H), 8.17 (d, 1H), 7.93-7.86 (m, 2H), 7.71 (s, 1H), 7.55-7.43 (m, 2H), 7.04 (d, 2H), 4.87 (s, 2H), 4.16 (s, 4H); MS (EI) for C₂₀H₇N₅O: 344 (MH⁺).

7-(H-imidazo[4,5-b]pyridin-6-yl)-4-pyrimidin-4-yl-2,3,4,5-tetrahydro-1,4-benzoxazepine. Synthesized according to the method of example 26 using triethylorthoformate in refluxing ethanol in step 3 and 4-chloropyrimidine in step 5. ¹H NMR (400 MHz, DMSO-d6): δ 8.65 (d, 1H), 8.51-8.46 (m, 2H), 8.23 (d, 1H), 8.17 (d, 1H), 7.96 (s, 1H), 7.59-7.55 (m, 1H), 7.07 (d, 2H), 4.88 (s, 2H), 4.18 (s, 4H), 1.85 (s, 8H); MS (EI) for C₁₉H₁₆N₆O: 343 (MH⁺).

6-(4-pyrido[3,2-d]pyrimidin-4-yl]-2,3,4,5-tetrahydro-1,4-benzoxazepin-7-yl)-1H-imidazo[4,5-b]pyridin-2-amine. Synthesized according to the method of example 26 using 4-chloropyrido[3,2-d]pyrimidine in step 5. ¹H NMR (400 MHz, MeOH-d4): δ 9.01 (d, 1H), 8.75 (br, 2H), 8.43 (s, 1H), 8.01 (s, 1H), 7.75 (m, 2H), 7.52 (brs, 1H), 7.01 (brs, 1H), 5.70 (s, 2H), 4.71 (s, 4H); MS (EI) for C₂₂H₁₈N₈O: 410.9 (MH⁺).

6-{4-[5-Methyl-6-(phenylamino)pyrimidin-4-yl]-2,3,4,5-tetrahydro-1,4-benzoxazepin-7-yl}-1H-imidazo[4,5-b]pyridine-2-amine. Synthesized according to the method of example 26 using 6-chloro-5-methyl-N-phenylpyrimidin-4-amine (reagent preparation 49) in step 5. ¹H NMR (400 MHz, dmso-d6) δ 12.03 (brs, 1H), 8.25 (s, 1H), 8.18 (s, 1H), 7.60 (m, 5H), 7.42 (d, 1H), 7.21 (t, 2H), 7.05 (d, 1H), 6.89 (t, 1H), 6.60 (br, 2H), 4.58 (s, 2H), 4.32 (s, 2H), 3.78 (s, 2H), 2.15 (s, 3H); MS (EI) for C₂₆H₂₄N₈O: 464.2 (MH⁺).

7-(1H-benzimidazol-6-yl)-4-(2-phenylquinazolin-4-yl)-2,3,4,5-tetrahydro-1,4-benzoxazepine. Synthesized according to the method of example 26 using 4-bromo-2-nitroaniline in step 1, triethylorthoformate in refluxing ethanol in step 3 and 4-chloro-2-phenylquinazoline in step 5. ¹H NMR (400 MHz, DMSO-d₆): 12.60-12.46 (m, 1H), 8.55-8.22 (m, 3H), 8.21-7.72 (m, 6H), 7.70-7.22 (m, 7H), 5.24 (s, br, 2H), 4.53 (s, br, 2H), 4.38 (s, br, 2H); MS (EI) for C₃₀H₂₃N₅O: 470.2 (MH⁺).

6-[4-(2-phenylquinazolin-4-yl)-2,3,4,5-tetrahydro-1,4-benzoxazepin-7-yl]-1H-benzimidazol-2-amine. Synthesized according to the method of example 26 using 4-bromo-2-nitroaniline in step 1, and 4-chloro-2-phenylquinazoline in step 5. ¹H NMR (400 MHz, DMSO-d₆): 8.36 (m, 2H), 8.25 (s, 1H), 8.21-8.11 (m, 2H), 7.92-7.73 (m, 3H), 7.65 (d, 1H), 7.612-7.36 (m, 4H), 7.30 (t, 2H), 6.91 (d, 2H), 6.70 (s, br, 2H), 5.22 (s, 2H), 4.52 (m, 2H), 4.37 (m, 2H); MS (EI) for C₃₀H₂₄N₆O: 486.1 (MH⁺).

4-(7-fluoroquinolin-4-yl)-7-(2-methyl-1H-benzimidazol-6-yl)-2,3,4,5-tetrahydro-1,4-benzoxazepine. Synthesized according to the method of example 26 using 4-bromo-2-nitroaniline in step 1, triethylorthoacetate in refluxing ethanol in step 3 and 4-chloro-7-fluoroquinoline in step 5. ¹H NMR (400 MHz, CD₃OD): 8.52 (d, 1H), 8.19-8.11 (m, 1H), 7.69 (s, 1H), 7.65-7.44 (m, 5H), 7.38-7.29 (m, 1H), 7.14-6.98 (m, 2H), 4.67 (s, 2H), 4.39 (m, 2H), 3.89 (m, 2H), 2.59 (s, 3H); MS (EI) for C₂₆H₂₁FN₄O 425.0 (MH⁺).

4-[7-(2-methyl-1H-benzimidazol-6-yl)-2,3-dihydro-1,4-benzoxazepin-4(5H)-yl]quinoline-7-carbonitrile. Synthesized according to the method of example 26 using 4-bromo-2-nitroaniline in step 1, triethylorthoacetate in refluxing ethanol in step 3 and 4-chloro-7-cyanoquinoline in step 5. ¹H NMR (400 MHz, CD₃OD): 8.64 (d, 1H), 8.22 (d, 1H), 8.07 (d, 1H), 7.82 (t, 1H), 7.71-7.34 (m, 6H), 7.09 (d, 1H), 4.85-4.52 (dd, 2H), 4.48-4.38 (m, 1H), 4.24-3.66 (m, 3H), 2.57 (s, 3H); MS (EI) for C₂₇H₂₁N₅O: 432.0 (MH⁺).

7-(2-methyl-1H-benzimidazol-6-yl)-4-[2-methyl-7-(methyloxy)quinazolin-4-yl]-2,3,4,5-tetrahydro-1,4-benzoxazepine. Synthesized according to the method of example 26 using 4-bromo-2-nitroaniline in step 1, triethylorthoacetate in refluxing ethanol in step 3 and 4-chloro-7-methoxy-2-methylquinazoline in step 5. ¹H NMR (400 MHz, CD₃OD): 8.20 (d, 2H), 7.92 (s, 1H), 7.87-7.78 (m, 3H), 7.58 (d, 1H), 7.26 (dd, 1H), 7.11-7.02 (m, 2H), 5.44 (s, 2H), 4.65-4.54 (m, 4H), 3.99 (s, 3H), 2.88 (s, 3H), 2.58 (s, 3H); MS (EI) for C₃₀H₂₄N₆O: 486.1 (MH⁺).

7-(1H-benzimidazol-6-yl)-4-[2-methyl-7-(methyloxy)quinazolin-4-yl]-2,3,4,5-tetrahydro-1,4-benzoxazepine. Synthesized according to the method of example 26 using 4-bromo-2-nitroaniline in step 1, triethylorthoformate in refluxing ethanol in step 3 and 4-chloro-7-methoxy-2-methylquinazoline in step 5. ¹H NMR (400 MHz, DMSO); δ 8.25 (s, 1H), 8.00-7.42 (m, 6H), 7.17-6.98 (m, 3H), 5.02 (s, 2H), 4.50-4.38 (m, 2H), 4.21-4.10 (m, 2H), 3.88 (s, 3H), 2.44 (s, 2H); MS (EI) for C26H23N5O2: 438.2 (MH⁺).

6-{4-[2-methyl-7-(methyloxy)quinazolin-4-yl]-2,3,4,5-tetrahydro-1,4-benzoxazepin-7-yl}-1H-imidazo[4,5-b]pyridin-2-amine. Synthesized according to the method of example 26 using 4-chloro-7-methoxy-2-methylquinazoline in step 5. ¹H NMR (400 MHz, DMSO); δ 8.15 (s, 1H), 7.90 (d, 1H), 7.71-7.42 (m, 3H), 7.17-6.95 (m, 3H), 6.70 (s, 2H), 5.00 (s, 2H), 4.48-4.38 (m, 2H), 4.20-4.09 (m, 2H), 3.87 (s, 3H), 2.44 (s, 3H); MS (EI) for C₂₅H₂₃N₇O₂: 454.2 (MH⁺).

6-{4-[2-methyl-7-(methyloxy)quinazolin-4-yl]-2,3,4,5-tetrahydro-1,4-benzoxazepin-7-yl}-1H-benzimidazol-2-amine. Synthesized according to the method of example 26 using 4-bromo-2-nitroaniline in step 1 and 4-chloro-7-methoxy-2-methylquinazoline in step 5. ¹H NMR (400 MHz, DMSO); δ 7.92 (d, 1H), 7.61-7.57 (m, 1H), 7.46-7.40 (m, 1H), 7.33 (s, 1H), 7.18-7.09 (m, 3H), 7.06-6.96 (m, 2H), 6.23 (s, 2H), 4.99 (s, 2H), 4.45-4.32 (m, 2H), 4.18-4.10 (m, 2H), 3.88 (s, 3H), 2.44 (s, 3H); MS (EI) for C₂₆H₂₄N₆O₂: 453.0 (MI).

7-(1H-imidazo[4,5-b]pyridin-6-yl)-4-[2-methyl-7-(methyloxy)quinazolin-4-yl]-2,3,4,5-tetrahydro-1,4-benzoxazepine. Synthesized according to the method of example 26 using triethylorthoformate in refluxing ethanol in step 3 and 4-chloro-7-methoxy-2-methylquinazoline in step 5. ¹H NMR (400 MHz, DMSO); δ 8.68-8.64 (m, 1H), 8.48 (s, 1H), 8.21 (s, 1H), 7.93-7.87 (d, 1H), 7.80-7.75 (m, 1H), 7.62-7.56 (m, 1H), 7.11 (d, 1H), 7.08-7.00 (m, 2H), 5.03 (s, 2H), 4.50-4.25 (m, 2H), 4.20-4.12 (m, 2H), 3.88 (s, 2H), 2.43 (s, 3H); MS (EI) for C25H₂₂N6O₂: 438.9 (MH⁺).

### Example 27

### methyl{6-[4-(6,6-dimethyl-5,6,7,8-tetrahydroquinazolin-4-yl)-2,3,4,5-tetrahydro-1,4-benzoxazepin-7-yl]-1H-imidazo[4,5-b]pyridin-2-yl}carbamate

STEP 1: A mixture of 1,1-dimethylethyl 7-(6-amino-5-nitropyridin-3-yl)-2,3-dihydro-1,4-benzoxazepine-4(5H)-carboxylate (3.5 g, 9.1 mmol, example 26, step 1) in methanol (75 mL) and 4N hydrogen chloride in dioxane (11 mL) was stirred at 50° C. for 1.5 h and then concentrated. The resulting residue was triturated with a 10% methanol in diethyl ether solution (50 mL) to provide 3-nitro-5-(2,3,4,5-tetrahydro-1,4-benzoxazepin-7-yl)pyridin-2-amine dihydrochloride (3.1 g, 95%) as a red solid. ¹H NMR (400 MHz, d₆-DMSO) δ 9.76 (bs, 2H), 8.80 (d, 1H), 8.60 (s, 1H), 7.90 (s, 1H), 7.73 (dd, 1H), 7.16 (d, 1H), 4.39 (bs, 2H), 4.25 (bs, 2H), 3.48 (bs, 2H); MS (EI) for C₁₄H₁₄N₄O₃: 287 (MH⁺).

STEP 2: A solution of 3-nitro-5-(2,3,4,5-tetrahydro-1,4-benzoxazepin-7-yl)pyridin-2-amine dihydrochloride (540 mg, 1.50 mmol), 4-chloro-6,6-dimethyl-5,6,7,8-tetrahydroquinazoline (270 mg, 1.37 mmol, reagent preparation 3), and diisopropylethylamine (970 mg, 7.49 mmol) in N-methylpyrrolidinone (3 mL) was stirred at 120° C. for 18 h. After cooling to room temperature ethyl acetate (100 mL) was added, the formed precipitate was filtered off, the organic filtrate was washed with saturated sodium bicarbonate (50 mL), water (2×50 mL), and brine (50 mL), dried over sodium sulfate, filtered and concentrated to afford crude 5-[4-(6,6-dimethyl-5,6,7,8-tetrahydroquinazolin-4-yl)-2,3,4,5-tetrahydro-1,4-benzoxazepin-7-yl]-3-nitropyridine-2-amine (0.5 g) as a brown solid which was used in the next step without further purification. MS (EI) for C₂₄H₂₆N₆O₃: 447 (MH⁺).

STEP 3: A mixture of 5-[4-(6,6-dimethyl-5,6,7,8-tetrahydroquinazolin-4-yl)-2,3,4,5-tetrahydro-1,4-benzoxazepin-7-yl]-3-nitropyridine-2-amine (0.5 g, 1.37 mmol) and palladium on carbon (0.5 g, 50% water) in methanol (50 mL) was hydrogenated in a Parr apparatus at 40 psi for 90 min. The mixture was filtered through celite and concentrated. Column chromatography of the residue on silica (dichloromethane/methanol 9:1) provided 5-[4-(6,6-dimethyl-5,6,7,8-tetrahydroquinazolin-4-yl)-2,3,4,5-tetrahydro-1,4-benzoxazepin-7-yl]pyridine-2,3-diamine (174 mg, 30% yield over 2 steps) as a brown solid. MS (EI) for C₂₄H₂₅N₆O: 417 (MH⁺).

STEP 4: A mixture of 5-[4-(6,6-dimethyl-5,6,7,8-tetrahydroquinazolin-4-yl)-2,3,4,5-tetrahydro-1,4-benzoxazepin-7-yl]pyridine-2,3-diamine (174 mg, 0.42 mmol) and 1,3-bis(methoxycarbonyl)-2-methyl-2-thiopseudourea (86 mg, 0.42 mmol) in acetic acid (5 mL) was stirred at 80° C. for 10 h. After cooling to room temperature the mixture was concentrated, methanol (10 mL) was added, the precipitate was filtered off, and lyophilized from a mixture of acetonitrile (2 mL), water (6 mL), and 1N hydrochloric acid (0.25 mL) to give the hydrochloride salt of the title Compound (107 mg, 48% yield) as a yellow solid. ¹H NMR (400 MHz, methanol-d₄): 8.53 (m, 2H), 8.35 (s, 1H), 7.78 (s, 1H), 7.58 (d, 1H), 7.10 (d, 1H), 5.18 (s, 2H), 4.49 (m, 2H), 4.34 (m, 2H), 3.93 (s, 3H), 2.86 (m, 2H), 2.61 (s, 2H), 1.71 (m, 2H), 0.95 (s, 6H); MS (EI) for C₂₇H₂₉N₇O₃: 500 (MH⁺).

Methyl{6-[4-(6,6-dimethyl-5,6,7,8-tetrahydroquinazolin-4-yl)-2,3,4,5-tetrahydro-1,4-benzoxazepin-7-yl]-1H-benzimidazol-2-yl}carbamate. Prepared according to the method of example 27 by 4-[4-(6,6-dimethyl-5,6,7,8-tetrahydroquinazolin-4-yl)-2,3,4,5-tetrahydro-1,4-benzoxazepin-7-yl]-2-nitroaniline in step 3. ¹H NMR (400 MHz, d₆-DMSO): 8.71 (s, 1H), 7.61 (s, 1H), 7.58-7.44 (m, 3H), 6.99 (d, 1H), 5.10 (s, 2H), 4.43 (m, 2H), 4.18 (m, 2H), 3.81 (s, 3H), 2.78 (t, 2H), 2.56 (s, 2H), 1.57 (t, 2H), 0.86 (s, 6H); MS (EI) for C₂₈H₃₀N₆O₃: 499 (MH⁺).

### Example 28

### ethyl{6-[4-(6,6-dimethyl-5,6,7,8-tetrahydroquinazolin-4-yl)-2,3,4,5-tetrahydro-1,4-benzoxazepin-7-yl]-1H-imidazo[4,5-b]pyridin-2-yl}carbamate

STEP 1: A solution of 5-[4-(6,6-dimethyl-5,6,7,8-tetrahydroquinazolin-4-yl)-2,3,4,5-tetrahydro-1,4-benzoxazepin-7-yl]pyridine-2,3-diamine (188 mg, 0.45 mmol, example 27, step 3) and ethyl isothiocyanatoformate (59 mg, 0.45 mmol) in dioxane (2 mL) was stirred at room temperature for 30 h. After 24 h and 48 h reaction time, additional ethyl isothiocyanatoformate (50 mg, 0.38 mmol) was added each time. The mixture was concentrated and the residue purified directly by column chromatography on silica (ethyl acetate) to give crude ethyl[1-({3-amino-5-[4-(6,6-dimethyl-5,6,7,8-tetrahydroquinazolin-4-yl)-2,3,4,5-tetrahydro-1,4-benzoxazepin-7-yl]pyridine-2-yl}amino)ethenyl]carbamate (82 mg) which was used in the next step without further purification. MS (EI) for C₂₈H₃₃N₇O₃S: 548 (MH⁺).

STEP 2: A mixture of ethyl[1-({3-amino-5-[4-(6,6-dimethyl-5,6,7,8-tetrahydroquinazolin-4-yl)-2,3,4,5-tetrahydro-1,4-benzoxazepin-7-yl]pyridine-2-yl}amino)ethenyl]carbamate (82 mg, 0.15 mmol) and mercury(II) oxide (33 mg, 0.15 mmol) in tetrahydrofuran (8 mL) was stirred at 70° C. for 24 h. On cooling to room temperature, the mixture filtered then concentrated and the residue purified by preparative reverse phase HPLC to afford the title Compound (6 mg, 3% yield over 2 steps) as a colorless solid. ¹H NMR (400 MHz, methanol-d₄): 8.51 (s, 1H), 8.49 (d, 1H), 8.22 (d, 1H), 7.72 (d, 1H), 7.66 (dd, 1H), 7.09 (d, 1H), 5.17 (s, 2H), 4.48 (m, 2H), 4.36 (q, 2H), 4.33 (m, 2H), 2.85 (m, 2H), 2.60 (s, 2H), 1.69 (m, 2H), 1.38 (t, 3H), 0.95 (s, 6H); MS (EI) for C₂₈H₃₁N₇O₃: 514 (MH⁺).

### Example 29

### methyl{6-[4-(6,6-dimethyl-5,6-dihydroquinazolin-4-yl)-2,3,4,5-tetrahydro-1,4-benzoxazepin-7-yl]-1H-imidazo[4,5-b]pyridin-2-yl}carbamate

STEP 1: To a solution of 3-nitro-5-(2,3,4,5-tetrahydro-1,4-benzoxazepin-7-yl)pyridin-2-amine dihydrochloride (1.0 g, 2.8 mmol) in acetic acid (20 mL) and ethanol (20 mL) was added Pd/C (10% wt/wt, 0.5 g) and the reaction mixture was stirred under H₂ (45 PSI) for 1 hour. The resulting pale yellow solution was filtered through Celite and the filtrate was concentrated to give 5-(2,3,4,5-tetrahydro-1,4-benzoxazepin-7-yl)pyridine-2,3-diamine dihydrochloride (0.92 g, 100%) as a yellow powder. MS (EI) for C₁₄H₁₆N₄O: 257.3 (MH⁺).

STEP 2: To a slurry of 5-(2,3,4,5-tetrahydro-1,4-benzoxazepin-7-yl)pyridine-2,3-diamine dihydrochloride (0.92 g, 2.8 mmol) and 4-chloro-6,6-dimethyl-5,6-dihydroquinazoline (0.55 g, 2.8 mmol) in NMP was added diisopropylethylamine (2.4 mL, 14 mmol) and the reaction mixture was heated (90° C.) for 12 hours. The resulting dark red solution was loaded directly on to a column of dry silica and elution with MeOH (w/8% NH₄OH v/v) in CH₂Cl₂ (0-5%) provided 5-[4-(6,6-dimethyl-5,6-dihydroquinazolin-4-yl)-2,3,4,5-tetrahydro-1,4-benzoxazepin-7-yl]pyridine-2,3-diamine (0.86, 74% yield) as a brown solid. MS (EI) for C₂₄H₂₆N₆O: 415.1 (MH⁺).

Step 3: To a solution of 5-[4-(6,6-dimethyl-5,6-dihydroquinazolin-4-yl)-2,3,4,5-tetrahydro-1,4-benzoxazepin-7-yl]pyridine-2,3-diamine (0.24 g, 0.58 mmol) in acetic acid (3 mL) was added 1,3-bis(methoxycarbonyl)-2-methyl-2-thiopseudourea (0.19 g, 0.93 mmol). The reaction mixture was heated (60° C.) for 12 h and then concentrated. Purification by preparative reverse phase HPLC followed by the formation of the dihydrochloride salt provided methyl{6-[4-(6,6-dimethyl-5,6-dihydroquinazolin-4-yl)-2,3,4,5-tetrahydro-1,4-benzoxazepin-7-yl]-1H-imidazo[4,5-b]pyridin-2-yl)}carbamate dihydrochloride (0.12 g, 38% yield) as a white solid. ¹H NMR (400 MHz, CD₃OD) δ 8.57-8.44 (m, 2H), 8.25 (bs, 1H), 7.73 (bs, 1H), 7.56 (dd, 1H), 7.09 (d, 1H), 6.59 (d, 1H), 6.35 (d, 1H), 5.15 (s, 2H), 4.53-4.42 (m, 2H), 4.33-4.21 (m, 2H), 3.91 (s, 3H), 2.92 (s, 2H), 1.09 (s, 6H); MS (ES) for C₂₇H₂₇N₇O₃: 498.6 (MH⁺).

### Example 30

### 6-{4-[(7S)-7-ethyl-5,6,7,8-tetrahydroquinazolin-4-yl]-2,3,4,5-tetrahydro-1,4-benzoxazepin-7-yl}-1H-imidazo[4,5-b]pyridin-2-amine

STEP 1: A mixture of 2-amino-5-bromo-3-nitropyridine (0.70 g, 3.2 mmol), (4-{[(1,1-dimethylethyl)oxy]carbonyl}-2,3,4,5-tetrahydro-1,4-benzoxazepin-7-yl)boronic acid (example 1, step 2) (1.0 g, 3.1 mmol), [1,1′-bis(diphenylphosphino)ferrocene]dichloropalladium(II) complex with dichloromethane (0.15 mg, 0.2 mmol), diisopropylethylamine (1.8 g, 14 mmol) in 50% aqueous 1,4-dioxane (40 mL) was degassed with nitrogen for 5 minutes and then stirred at 90° C. for one hour. The reaction mixture was cooled to room temperature, diluted with ethyl acetate (80 mL) then filtered over celite. The filtrate was washed twice with brine (50 mL), filtered and the filtrate dried over sodium sulfate, filtered again and concentrated. The residue was purified by silica gel chromatography (25% to 95% ethyl acetate in hexanes gradient) to give 1,1-dimethylethyl 7-(6-amino-5-nitropyridin-3-yl)-2,3-dihydro-1,4-benzoxazepine-4(5H)-carboxylate (0.58 g, 48% yield); MS (EI) for C₁₉H₂₂N₄O₅: 389 (MH⁺).

STEP 2: A mixture of 1,1-dimethylethyl 7-(6-amino-5-nitropyridin-3-yl)-2,3-dihydro-1,4-benzoxazepine-4(5H)-carboxylate (0.58 g, 1.5 mmol), palladium (10% on charcoal, 0.50 g) and methanol (30 mL) was hydrogenated in a Parr apparatus at 45 psi for 18 hours. The mixture was filtered then concentrated and dried to give 1,1-dimethylethyl 7-(5,6-diaminopyridin-3-yl)-2,3-dihydro-1,4-benzoxazepine-4(5H)-carboxylate (0.51 g, 96% yield), MS (EI) for C₁₉H₂₄N₄O₃: 357 (MH⁺).

STEP 3: To a solution of 1,1-dimethylethyl 7-(5,6-diaminopyridin-3-yl)-2,3-dihydro-1,4-benzoxazepine-4(5H)-carboxylate (0.51 g, 1.4 mmol) in acetic acid (5 mL) was added 1,3-bis(methoxycarbonyl)-2-methyl-2-thiopseudourea (0.3 g, 1.4 mmol). The reaction mixture was heated 65° C. for 18 h and then concentrated. The resulting residue was suspended in water and basified with portion wise addition of solid sodium bicarbonate. After complete neutralization of the aqueous mixture the insoluble solid was collected by filtration and washed with water then 50% ethyl acetate in hexanes and the filter cake dried to give 1,1-dimethylethyl 7-(2-{[(methyloxy)carbonyl]amino}-1H-imidazo[4,5-b]pyridin-6-yl)-2,3-dihydro-1,4-benzoxazepine-4(5H)-carboxylate (0.52 g, 83% yield), MS (EI) for C₂₂H₂₅N₅O₅: 440 (MH⁺).

STEP 4: To a mixture of 1,1-dimethylethyl 7-(2-{[(methyloxy)carbonyl]amino}-3H-imidazo[4,5-b]pyridin-6-yl)-2,3-dihydro-1,4-benzoxazepine-4(5H)-carboxylate (0.52 g, 1.2 mmol) was taken into acetonitrile (5 mL) followed by addition of 4M hydrogen chloride in 1,4-dioxane (5 mL) and the mixture was stirred at room temperature for 10 minutes. The reaction mixture was concentrated to give a white solid. It was washed with ether then dried to give methyl[6-(2,3,4,5-tetrahydro-1,4-benzoxazepin-7-yl)-1H-imidazo[4,5-b]pyridin-2-yl]carbamate hydrochloride salt (0.40 g, 100% yield), MS (EI) for C₇H₁₇N₅O₃: 340 (MH⁺).

STEP 5: A mixture of methyl[6-(2,3,4,5-tetrahydro-1,4-benzoxazepin-7-yl)-1H-imidazo[4,5-b]pyridin-2-yl]carbamate hydrochloride (84 mg, 0.23 mmol, (7S)-4-chloro-7-ethyl-5,6,7,8-tetrahydroquinazoline (reagent preparation 3) (35 mg, 0.18 mmol) and N,N-diisopropylethylamine (0.15 mL, 0.90 mmol) in N-methyl-2-pyrrolidone (2.0 mL) was reacted in a microwave apparatus (250 W) for 5 min. at 110° C. After cooling to room temperature the reaction mixture was diluted with methanol (2 mL) and purified by preparative reverse phase HPLC (0.1% aqueous ammonium acetate-acetonitrile) to give 6-{4-[(7S)-7-ethyl-5,6,7,8-tetrahydroquinazolin-4-yl]-2,3,4,5-tetrahydro-1,4-benzoxazepin-7-yl}-1H-imidazo[4,5-b]pyridin-2-amine. ¹H NMR (400 MHz, Methanol-d₄): 8.33 (s, 1H), 8.13 (brs, 1H), 7.63 (s, 1H), 7.50 (brs, 1H), 7.31 (d, 1H), 7.04 (d, 1H), 4.75 (b, 2H), 4.44 (m, 1H), 4.24 (m, 1H), 3.99 to 3.86 (m, 2H), 2.94 to 2.86 (m, 2H), 2.60 (m, 1H), 2.28 (m, 1H), 1.97 (m, 1H), 1.75 (m, 1H), 1.36 (m, 2H), 1.14 (m, 1H), 0.97 (t, 3H); MS (EI) for C₂₅H₂₇N₇O: 442 (MH⁺).

TABLE 2
The following compounds were prepared using the procedures described
herein.

| Compound Name | NMR | MS |
|---|---|---|
| 1-(6,6-dimethyl-4-{7-[4- | ¹H NMR (400 MHz, DMSO-d6) δ | MS (EI) for |
| (methyloxy)-3-{[2- | 7.61 (d, 1H), 7.44 (dd, 1H), | C₃₂H₄₂N₄O₄: |
| (methyloxy)ethyl]oxy}phenyl]- | 7.20-7.14 (m, 2H), 7.02 (d, 1H), | 547 (MH⁺) |
| 2,3-dihydro-1,4-benzoxazepin- | 6.97 (d, 1H), 4.60 (s, 2H), | |
| 4(5H)-yl}-5,6,7,8- | 4.31-4.24 (m, 2H), 4.18-4.13 (m, 2H), | |
| tetrahydroquinazolin-2-yl)-N,N- | 3.87-3.82 (m, 2H), 3.79 (s, 3H), | |
| dimethylmethanamine | 3.70-3.66 (m, 2H), 3.33 (s, 3H), 2.69 (t, | |
| | 2H), 2.45 (s, 2H), 2.14 (s, 6H), | |
| | 1.59 (t, 2H), 0.86 (s, 6H) | |
| 1-{4-[7-{3- | ¹H NMR (400 MHz, DMSO-d6) δ | MS (EI) for |
| [(difluoromethyl)oxy]-4- | 7.66 (d, 1H), 7.54 (dd, 1H), | C₃₀H₃₆F₂N₄O₃: |
| (methyloxy)phenyl}-2,3- | 7.51-7.44 (m, 2H), 7.24 (d, 1H), 7.13 (t, | 539 (MH⁺) |
| dihydro-1,4-benzoxazepin- | 3H), 7.00 (d, 1H), 4.74 (br s, 2H), | |
| 4(5H)-yl]-6,6-dimethyl-5,6,7,8- | 4.41-4.32 (m, 2H), 4.31-4.18 (m, | |
| tetrahydroquinazolin-2-yl}- | 2H), 3.97-3.89 (m, 2H), 3.87 (s, | |
| N,N-dimethylmethanamine. | 3H), 2.84-2.69 (m, 8H), 2.48 (s, | |
| | 2H), 1.62 (t, 2H), 0.85 (s, 6H) | |
| 1-[5-(4-{2- | ¹H NMR (400 MHz, DMSO-d6) δ | MS (EI) for |
| [(dimethylamino)methyl]-6,6- | 7.84-7.78 (m, 2H), 7.62 (d, 1H), | C₃₁H₃₈N₄O₃: |
| dimethyl-5,6,7,8- | 7.44 (dd, 1H), 7.26 (d, 1H), | 515 (MH⁺) |
| tetrahydroquinazolin-4-yl}- | 6.99 (d, 1H), 4.62 (s, 2H), | |
| 2,3,4,5-tetrahydro-1,4- | 4.33-4.25 (m, 2H), 3.93 (s, 3H), | |
| benzoxazepin-7-yl)-2- | 3.88-3.82 (m, 2H), 3.39 (br s, 2H), 2.69 (t, | |
| (methyloxy)phenyl]ethanone. | 2H), 2.57 (s, 3H), 2.44 (s, 2H), | |
| | 2.15 (s, 6H), 1.59 (t, 2H), 0.86 (s, | |
| | 6H) | |
| 1-(6,6-dimethyl-4-{7-[4- | ¹H NMR (400 MHz, DMSO-d6) δ | MS (EI) for |
| (methyloxy)-3- | 8.00 (d, 1H), 7.96 (dd, 1H), | C₃₀H₃₈N₄O₄S: |
| (methylsulfonyl)phenyl]-2,3- | 7.63 (d, 1H), 7.45 (dd, 1H), 7.40 (d, | 551 (MH⁺) |
| dihydro-1,4-benzoxazepin- | 1H), 7.01 (d, 1H), 4.66 (s, 2H), | |
| 4(5H)-yl}-5,6,7,8- | 4.34-4.28 (m, 2H), 4.00 (s, 3H), | |
| tetrahydroquinazolin-2-yl)-N,N- | 3.90-3.83 (m, 2H), 3.41 (br s, 2H), | |
| dimethylmethanamine. | 3.28 (s, 3H), 2.68 (t, 2H), 2.43 (s, | |
| | 2H), 2.16 (br s, 7H), 1.59 (t, 2H), | |
| | 0.85 (s, 6H) | |
| N-[5-(4-{2- | ¹H NMR (400 MHz, DMSO-d6) δ | MS (EI) for |
| [(dimethylamino)methyl]-6- | 9.03 (s, 1H), 7.51 (d, 1H), | C₂₈H₃₇N₅O₄S: |
| methyl-5-(1- | 7.49-7.39 (m, 3H), 7.17 (d, 1H), | 540 (MH⁺) |
| methylethyl)pyrimidin-4-yl}- | 7.02 (d, 1H), 4.45 (s, 2H), | |
| 2,3,4,5-tetrahydro-1,4- | 4.33-4.26 (m, 2H), 3.87 (s, 3H), | |
| benzoxazepin-7-yl)-2- | 3.74-3.66 (m, 2H), 3.32 (s, 2H), | |
| (methyloxy)phenyl]methanesulfonamide. | 3.30-3.21 (m, 1H), 2.98 (s, 3H), 2.48 (s, | |
| | 3H), 2.30 (br s, 6H), 1.32 (d, 6H) | |
| N′-{5-[(4-fluorophenyl)methyl]- | ¹H NMR (400 MHz, CD₃OD) δ | MS (EI) for |
| 4-methyl-6-[7-(2-methyl-1H- | 7.58-7.47 (m, 2H), 7.42 (dd, 1H), | C₃₃H₃₆FN₇O: |
| benzimidazol-5-yl)-2,3-dihydro- | 7.25 (dd, 1H), 7.15-7.08 (m, 2H), | 566 (MH⁺) |
| 1,4-benzoxazepin-4(5H)- | 7.01-6.92 (m, 3H), 6.86 (s, 1H), | |
| yl]pyrimidin-2-yl}-N,N- | 4.49 (s, 2H), 4.28-4.22 (m, 2H), | |
| dimethylethane-1,2-diamine | 3.90 (s, 2H), 3.84-3.78 (m, 2H), | |
| | 3.45 (t, 2H), 2.63-2.53 (m, 5H), | |
| | 2.34 (s, 6H), 2.06 (s, 3H) | |
| 2-fluoro-N-({4-methyl-6-[7-(2- | ¹H NMR (400 MHz, DMSO-d6) δ | MS (EI) for |
| methyl-1H-benzimidazol-5-yl)- | 7.63 (s, 1H), 7.55 (s, 1H), 7.50 (d, | C₂₈H₃₃FN₆O: |
| 2,3-dihydro-1,4-benzoxazepin- | 2H), 7.37 (d, 1H), 7.02 (d, 1H), | 489 (MH⁺) |
| 4(5H)-yl]-5-(1- | 4.45 (dd, 3H), 4.36-4.22 (m, | |
| methylethyl)pyrimidin-2- | 3H), 3.71 (s, 2H), 3.63 (s, 2H), | |
| yl}methyl)ethanamine | 3.40-3.21 (m, 1H), 2.76 (t, 1H), | |
| | 2.69 (t, 1H), 2.47 (s, 3H), 1.87 (s, | |
| | 3H), 1.33 (d, 6H) | |
| 6-{4-[2-{[(2- | ¹H NMR (400 MHz, DMSO-d6) δ | MS (EI) for |
| fluoroethyl)amino]methyl}-6- | 8.37 (d, 1H), 7.88 (s, 2H), 7.81 (d, | C₂₆H₃₀FN₇O: |
| methyl-5-(1- | 1H), 7.63 (s, 1H), 7.57 (d, 1H), | 508 (MH⁺) |
| methylethyl)pyrimidin-4-yl]- | 7.04 (d, 1H), 4.51 (s, 2H), 4.43 (t, | |
| 2,3,4,5-tetrahydro-1,4- | 1H), 4.31 (s, 3H), 3.71 (s, 2H), | |
| benzoxazepin-7- | 3.61 (s, 2H), 3.24 (s, 1H), 2.74 (t, | |
| yl}[1,3]thiazolo[5,4-b]pyridin- | 1H), 2.67 (s, 1H), 2.46 (s, 3H), | |
| 2-amine. | 1.31 (d, 6H) | |
| N,N-dimethyl-1-{4-methyl-6- | ¹H NMR (400 MHz, DMSO-d6) δ | MS (EI) for |
| [7-(2-methyl-1H-benzimidazol- | 7.61 (d, 2H), 7.46 (dd, 2H), | C₂₈H₃₄N₆O: |
| 6-yl)-2,3-dihydro-1,4- | 7.37 (d, 1H), 6.99 (d, 1H), 4.61 (s, 2H), | 471 (MH⁺) |
| benzoxazepin-4(5H)-yl]-5- | 4.32 (s, 2H), 3.78 (s, 2H), 3.34 (br | |
| propylpyrimidin-2- | s, 2H), 2.50 (m, 5H), 2.35 (s, 3H), | |
| yl}methanamine | 2.12 (s, 6H), 1.45 (d, 2H), 0.75 (t, | |
| | 3H) | |
| N,N-dimethyl-1-{4-methyl-6- | ¹H NMR (400 MHz, DMSO-d6) δ | MS (EI) for |
| [7-(2-methyl-1H-benzimidazol- | 7.61 (d, 1H), 7.55-7.41 (m, 3H), | C₂₈H₃₂N₆O: |
| 6-yl)-2,3-dihydro-1,4- | 7.35 (dt, 1H), 6.97 (dd, 1H), | 469 (MH⁺) |
| benzoxazepin-4(5H)-yl]-5-prop- | 6.23-6.01 (m, 1H), 5.25 (t, 1H), | |
| 2-en-1-ylpyrimidin-2- | 4.93 (t, 1H), 4.61 (s, 2H), 4.27 (d, 2H), | |
| yl}methanamine | 3.82 (s, 2H), 3.37 (s, 2H), 3.32 (s, | |
| | 2H), 2.50 (m, 3H), 2.27 (s, 3H), | |
| | 2.15 (s, 6H) | |
| 6-(4-{2- | ¹H NMR (400 MHz, DMSO-d6) δ | MS (EI) for |
| [(dimethylamino)methyl]-6- | 8.38 (d, 1H), 7.88 (s, 2H), 7.82 (d, | C₂₆H₃₁N₇OS: |
| methyl-5-propylpyrimidin-4- | 1H), 7.70 (s, 1H), 7.53 (d, 1H), | 490 (MH⁺) |
| yl}-2,3,4,5-tetrahydro-1,4- | 7.01 (d, 1H), 4.63 (s, 2H), 4.34 (s, | |
| benzoxazepin-7- | 2H), 3.78 (s, 2H), 3.33 (d, 2H), | |
| yl)[1,3]thiazolo[5,4-b]pyridin- | 2.51 (m, 2H), 2.34 (s, 3H), 2.11 (s, | |
| 2-amine | 6H), 1.46 (s, 2H), 0.75 (t, 3H) | |
| 6-(4-{2- | ¹H NMR (400 MHz, DMSO-d6) δ | MS (EI) for |
| [(dimethylamino)methyl]-6- | 8.36 (d, 1H), 7.88 (s, 2H), 7.80 (d, | C₂₆H₂₉N₇OS: |
| methyl-5-prop-2-en-1- | 1H), 7.60 (d, 1H), 7.53 (dd, 1H), | 488 (MH⁺) |
| ylpyrimidin-4-yl}-2,3,4,5- | 7.02 (d, 1H), 6.10 (dd, 1H), | |
| tetrahydro-1,4-benzoxazepin-7- | 5.23 (d, 1H), 4.94 (d, 1H), 4.63 (s, 2H), | |
| yl)[1,3]thiazolo[5,4-b]pyridin- | 4.28 (s, 2H), 3.83 (s, 2H), 3.34 (s, | |
| 2-amine | 2H), 3.30 (s, 2H), 2.26 (s, 3H), | |
| | 2.13 (s, 6H) | |
| N,N-dimethyl-1-{4-methyl-6- | ¹H NMR (400 MHz, DMSO-d6) δ | MS (EI) for |
| [7-(2-methyl-1H-benzimidazol- | 7.73-7.42 (m, 4H), | C₃₁H₃₂N₆O: |
| 5-yl)-2,3-dihydro-1,4- | 7.41-7.27 (m, 5H), 7.23 (d, 1H), 6.92 (dd, | 505.0 (MH⁺) |
| benzoxazepin-4(5H)-yl]-5- | 1H), 4.51 (d, 2H), 4.07-3.91 (m, | |
| phenylpyrimidin-2- | 2H), 3.53 (d, 2H), 3.38 (d2H), | |
| yl}methanamine | 2.49 (s, 3H), 2.16 (d, 6H), 2.02 (d, | |
| | 3H) | |
| 6-(4-{2- | ¹H NMR (400 MHz, DMSO-d6) δ | MS (EI) for |
| [(dimethylamino)methyl]-6- | 8.34 (d, 1H), 7.89 (s, 2H), 7.78 (d, | C₂₉H₂₉N₇OS: |
| methyl-5-phenylpyrimidin-4- | 1H), 7.52-7.39 (m, 3H), 7.32 (t, | 523.9 (MH⁺) |
| yl}-2,3,4,5-tetrahydro-1,4- | 4H), 6.95 (d, 1H), 4.57 (s, 2H), | |
| benzoxazepin-7- | 4.04 (s, 2H), 3.48 (s, 2H), 3.36 (d, | |
| yl)[1,3]thiazolo[5,4-b]pyridin- | 2H), 2.15 (s, 6H), 2.01 (s, 3H) | |
| 2-amine | | |
| N,N-dimethyl-1-{4-methyl-6- | ¹H NMR (400 MHz, DMSO-d6) | MS (EI) for |
| [7-(2-methyl-1H-benzimidazol- | δ12.22 (s, 1H), 7.67 (s, 1H), | C₂₉H₃₆N₆O: |
| 5-yl)-2,3-dihydro-1,4- | 7.59 (d, 1H), 7.56-7.39 (m, 2H), | 485.0 (MH⁺) |
| benzoxazepin-4(5H)-yl]-5-(2- | 7.34 (d, 1H), 6.96 (d, 1H), 4.56 (s, 2H), | |
| methylpropyl)pyrimidin-2- | 4.29 (s, 2H), 3.73 (s, 2H), 2.60 (t, | |
| yl}methanamine | 2H), 2.32 (s, 3H), 2.10 (s, 6H), | |
| | 1.88 (s, 2H, OAc), 1.76-1.53 (m, | |
| | 1H), 0.52 (d 6H) | |
| 6-(4-{5-(cyclopropylmethyl)-2- | ¹H NMR (400 MHz, DMSO-d6) | MS (EI) for |
| [(dimethylamino)methyl]-6- | δ 8.42 (d, 1H), 7.93 (s, 2H), | C₂₇H₃₁N₇OS: |
| methylpyrimidin-4-yl}-2,3,4,5- | 7.87 (d, 1H), 7.76 (d, 1H), 7.58 (dd, | 502.0 (MH⁺) |
| tetrahydro-1,4-benzoxazepin-7- | 1H), 7.07 (d, 1H), 4.70 (d, 2H), | |
| yl)[1,3]thiazolo[5,4-b]pyridin- | 4.37 (s, 2H), 3.85 (s, 2H), | |
| 2-amine | 3.50-3.42 (m, 2H), 2.74-2.58 (m, 2H), | |
| | 2.42 (d, 3H), 2.28-2.03 (m, 6H), | |
| | 0.90 (s, 1H), 0.48-0.31 (m, 2H), | |
| | 0.14-−0.28 (m, 2H) | |
| 6-(4-{2- | ¹H NMR (400 MHz, DMSO-d6) δ | MS (EI) for |
| [(dimethylamino)methyl]-6- | 8.37 (d, 1H), 7.88 (s, 2H), 7.81 (d, | C₂₇H₃₃N₇OS: |
| methyl-5-(2- | 1H), 7.72 (s, 1H), 7.53 (d, 1H), | 504.0 (MH⁺) |
| methylpropyl)pyrimidin-4-yl}- | 7.01 (d, 1H), 4.61 (s, 2H), 4.33 (s, | |
| 2,3,4,5-tetrahydro-1,4- | 2H), 3.76 (s, 2H), 3.37 (s, 2H), | |
| benzoxazepin-7- | 2.59 (d, 2H), 2.35 (s, 3H), 2.14 (s, | |
| yl)[1,3]thiazolo[5,4-b]pyridin- | 6H), 1.79-1.53 (m, 1H), 0.55 (d, | |
| 2-amine | 6H) | |
| 7-(2-methyl-1H-benzimidazol- | ¹H NMR (400 MHz, DMSO-d6) δ | MS (EI) for |
| 5-yl)-4-[6-methyl-5-(1- | 7.60 (s, 1H), 7.52-7.43 (m, 3H), | C₃₀H₃₆N₆O: |
| methylethyl)-2-(pyrrolidin-1- | 7.33 (dt, 1H), 6.99 (t, 1H), 4.40 (s, | 497.2 (MH⁺) |
| ylmethyl)pyrimidin-4-yl]- | 2H), 4.27 (d, 2H), 3.65 (br s, 2H), | |
| 2,3,4,5-tetrahydro-1,4- | 3.47 (s, 2H), 3.28 (dt, 1H), 2.47 (s, | |
| benzoxazepine | 3H), 2.44 (s, 3H), 2.41 (m, 4H), | |
| | 1.85 (s, 6H, OAc), 1.64-1.49 (m, | |
| | 4H), 1.30 (d, 6H) | |
| 1-{6,6-dimethyl-4-[7-(2- | ¹H NMR (400 MHz, dmso) δ | MS (ES) for |
| methyl-1H-benzimidazol-5-yl)- | 8.60 (s, 2H), 8.02-7.89 (m, 2H), | C₂₉H₃₄N₆O: |
| 2,3-dihydro-1,4-benzoxazepin- | 7.85 (d, 1H), 7.82 (d, 1H), 7.59 (d, 1H), | 483 (MH⁺) |
| 4(5H)-yl]-5,6,7,8- | 7.05 (d, 1H), 5.02 (s, 2H), | |
| tetrahydroquinazolin-2- | 4.53-4.29 (m, 3H), 4.24-3.99 (m, 2H), | |
| yl}ethanamine | 2.83 (s, 3H), 2.79 (t, 2H), 2.52 (s, | |
| | 2H), 1.60 (t, 2H), 1.44 (d, 3H), | |
| | 0.86 (s, 6H) | |
| 6-[4-(2,6,6-trimethyl-5,6,7,8- | ¹H NMR (400 MHz, dmso) δ | MS (ES) for |
| tetrahydroquinazolin-4-yl)- | 8.38 (d, 1H), 7.89 (s, 2H), 7.83 (d, 1H), | C₂₆H₂₈N₆OS: |
| 2,3,4,5-tetrahydro-1,4- | 7.73 (d, 1H), 7.56 (dd, 1H), | 473 (MH⁺) |
| benzoxazepin-7- | 7.05 (d, 1H), 4.59 (s, 2H), 4.29 (m, | |
| yl][1,3]thiazolo[5,4-b]pyridin- | 2H), 3.84 (m, 2H), 2.66 (t, 2H), | |
| 2-amine | 2.42 (s, 2H), 2.34 (s, 3H), 1.58 (t, | |
| | 2H), 0.84 (s, 6H) | |
| 4-[2-(fluoromethyl)-6,6- | ¹H NMR (400 MHz, dmso) δ | MS (ES) for |
| dimethyl-5,6,7,8- | 7.69 (s, 1H), 7.67 (d, 1H), 7.54 (d, 1H), | C₂₈H₃₀FN₅O: |
| tetrahydroquinazolin-4-yl]-7-(2- | 7.50 (dd, 1H), 7.44 (dd, 1H), | 472 (MH⁺) |
| methyl-1H-benzimidazol-5-yl)- | 7.03 (d, 1H), 5.23 (d, 2H), 4.66 (s, 2H), | |
| 2,3,4,5-tetrahydro-1,4- | 4.30 (m, 2H), 3.89 (s, 1H), 2.73 (t, | |
| benzoxazepine | 2H), 2.54 (s, 3H), 1.91 (s, 2H), | |
| | 1.61 (t, 2H), 0.86 (s, 6H) | |
| 6-{4-[2-methyl-7- | ¹H NMR (400 MHz, dmso) δ | MS (EI) |
| (methyloxy)quinazolin-4-yl]- | 8.45 (d, 1H), 8.25-8.1 (br, 3H), | Calculated for |
| 2,3,4,5-tetrahydro-1,4- | 7.89 (d, 2H), 7.59 (s, 1H), | C₂₅H₂₂N₆O₂S: |
| benzoxazepin-7- | 7.34-7.19 (m, 2H), 7.01 (d, 1H), | 470.6 Found: |
| yl}[1,3]thiazolo[5,4-b]pyridin- | 4.72-4.32 (m, 6H), 3.95 (s, 3H), 2.56 (s, 3H) | 471.2 (MH⁺) |
| 2-amine. Prepared as a | | |
| dihydrochloride salt. | | |
| 6-(4-{5-chloro-2- | ¹H NMR (400 MHz, methanol- | MS (EI) for |
| [(dimethylamino)methyl]-6- | d₄): 8.34 (d, 1H), 7.81 (d, 1H), | C₂₃H₂₄ClN₇OS: |
| methylpyrimidin-4-yl}-2,3,4,5- | 7.65 (d, 1H), 7.48 (dd, 1H), | 482 (MH⁺) |
| tetrahydro-1,4-benzoxazepin-7- | 7.06 (d, 1H), 5.07 (s, 2H), 4.36 (m, | |
| yl)[1,3]thiazolo[5,4-b]pyridin- | 2H), 4.23 (m, 2H), 3.76 (s, 2H), | |
| 2-amine. Prepared as an acetate | 2.50 (s, 3H), 2.47 (s, 6H), 1.94 (s, | |
| salt. | 3H) | |
| 6-(4-{5-bromo-2- | ¹H NMR (400 MHz, methanol- | MS (EI) for |
| [(dimethylamino)methyl]-6- | d₄): 8.35 (d, 1H), 7.81 (d, 1H), | C₂₃H₂₄BrN₇OS: |
| methylpyrimidin-4-yl}-2,3,4,5- | 7.65 (d, 1H), 7.48 (dd, 1H), | 526 (MH⁺) |
| tetrahydro-1,4-benzoxazepin-7- | 7.05 (d, 1H), 5.06 (s, 2H), 4.38 (m, | |
| yl)[1,3]thiazolo[5,4-b]pyridin- | 2H), 4.19 (m, 2H), 3.78 (s, 2H), | |
| 2-amine. Prepared as a | 2.56 (s, 3H), 2.48 (s, 6H), 1.94 (s, | |
| diacetate salt. | 3H) | |
| 1-{5-chloro-4-methyl-6-[7-(2- | ¹H NMR (400 MHz, methanol- | MS (EI) for |
| methyl-1H-benzimidazol-5-yl)- | d₄): 7.63 (s, 1H), 7.61 (d, 1H), | C₂₅H₂₇ClN₆O: |
| 2,3-dihydro-1,4-benzoxazepin- | 7.52 (d, 1H), 7.43 (m, 2H), | 463 (MH⁺) |
| 4(5H)-yl]pyrimidin-2-yl}-N,N- | 7.02 (d, 1H), 5.08 (s, 2H), 4.34 (m, | |
| dimethylmethanamine. | 2H), 4.23 (m, 2H), 3.84 (s, 2H), | |
| Prepared as a diacetate salt. | 2.58 (s, 3H), 2.52 (s, 6H), 2.50 (s, | |
| | 3H), 1.94 (s, 6H) | |
| 1-{5-bromo-4-methyl-6-[7-(2- | ¹H NMR (400 MHz, methanol- | MS (EI) for |
| methyl-1H-benzimidazol-5-yl)- | d₄): 7.64 (d, 1H), 7.61 (d, 1H), | C₂₅H₂₇BrN₆O: |
| 2,3-dihydro-1,4-benzoxazepin- | 7.52 (d, 1H), 7.43 (m, 2H), | 507 (MH⁺) |
| 4(5H)-yl]pyrimidin-2-yl}-N,N- | 7.00 (d, 1H), 5.06 (s, 2H), 4.36 (m, | |
| dimethylmethanamine. | 2H), 4.19 (m, 2H), 3.79 (s, 2H), | |
| Prepared as a diacetate salt. | 2.58 (s, 3H), 2.56 (s, 3H), 2.47 (s, | |
| | 6H), 1.93 (s, 6H) | |
| 4-[7-(1H-benzimidazol-5-yl)- | ¹H NMR (400 MHz, DMSO) δ | MS (EI) for |
| 2,3-dihydro-1,4-benzoxazepin- | 8.22 (s, 1H), 7.81 (s, 2H), 7.62 (s, | C₂₁H₂₀N₆O: |
| 4(5H)-yl]-6-methylpyrimidin-2- | 1H), 7.45 (dd, 2H), 7.00 (d, 1H), | 373.2 (MH⁺) |
| amine | 6.09 (s, 1H), 5.94 (s, 2H), 4.73 (s, | |
| | 2H), 4.03 (dd, 4H), 2.03 (d, 3H) | |
| 5-(4-fluorobenzyl)-4-[7-(3H- | ¹H NMR (400 MHz, DMSO) δ | MS (EI) for |
| imidazo[4,5-b]pyridin-6-yl)- | 8.47 (dd, 2H), 8.12-7.95 (d, 1H), | C₂₇H₂₄FN₇O: |
| 2,3-dihydro-1,4-benzoxazepin- | 7.53 (s, 1H), 7.12 (t, 2H), 7.05 (t, | 482.2 (MH⁺) |
| 4(5H)-yl]-6-methylpyrimidin-2- | 3H), 6.88 (d, 1H), 6.13 (s, 2H), | |
| amine | 4.34 (s, 2H), 4.22 (s, 2H), 3.83 (s, | |
| | 2H), 3.65 (s, 2H), 1.97 (s, 3H) | |
| 5-(4-fluorobenzyl)-4-methyl-6- | ¹H NMR (400 MHz, DMSO) δ | MS (EI) for |
| [7-(2-methyl-3H-imidazo[4,5- | 8.29 (s, 1H), 7.94 (s, 1H), 7.50 (d, | C₂₈H₂₆FN₇O: |
| b]pyridin-6-yl)-2,3-dihydro-1,4- | 1H), 7.11 (t, 2H), 7.05 (t, 3H), | 496.2 (MH⁺) |
| benzoxazepin-4(5H)- | 6.86 (s, 1H), 6.12 (s, 2H), 4.33 (s, | |
| yl]pyrimidin-2-amine | 2H), 4.22 (s, 2H), 3.83 (s, 2H), | |
| | 3.65 (s, 2H), 2.54 (s, 3H), 1.89 (s, | |
| | 3H) | |
| 1-{4-[7-(3H-imidazo[4,5- | ¹H NMR (400 MHz, DMSO) δ | MS (EI) for |
| b]pyridin-6-yl)-2,3-dihydro-1,4- | 8.62 (t, 1H), 8.45 (d, 1H), 8.17 (d, | C₂₇H₂₇N₇O₂: |
| benzoxazepin-4(5H)-yl]-7- | 1H), 7.90 (t, 1H), 7.74 (t, 1H), | 482.3 (MH⁺) |
| methoxyquinazolin-2-yl}-N,N- | 7.52 (dt, 1H), 7.12 (t, 1H), | |
| dimethylmethanamine | 7.05 (dd, 1H), 6.98 (d, 1H), 5.04 (s, | |
| | 2H), 4.44 (d, 2H), 4.18 (s, 2H), | |
| | 3.86 (s, 3H), 3.41 (s, 2H), 2.08 (d, | |
| | 6H) | |
| 6-{4-[2-amino-5-(4- | ¹H NMR (400 MHz, DMSO) δ | MS (EI) for |
| fluorobenzyl)-6- | 8.13 (d, 1H), 7.82 (s, 2H), 7.62 (d, | C27H24FN7OS: |
| methylpyrimidin-4-yl]-2,3,4,5- | 1H), 7.44 (dt, 1H), 7.09-6.92 (m, | 514.2 (MH⁺) |
| tetrahydro-1,4-benzoxazepin-7- | 5H), 6.83 (d, 1H), 6.05 (s, 2H), | |
| yl}[1,3]thiazolo[5,4-b]pyridin- | 4.27 (s, 2H), 4.14 (s, 2H), 3.72 (d, | |
| 2-amine | 2H), 3.55 (d, 2H), 1.91 (d, 3H) | |
| 4-[7-(1H-benzimidazol-5-yl)- | ¹H NMR (400 MHz, DMSO-d₆) δ | MS (EI) for |
| 2,3-dihydro-1,4-benzoxazepin- | 8.24 (d, 1H), 7.92-7.50 (m, 1H), | C₂₈H₂₅FN₆O: |
| 4(5H)-yl]-5-[(4- | 7.47 (d, 1H), 7.20 (d, 1H), | 481.0 (MH⁺) |
| fluorophenyl)methyl]-6- | 7.12 (dq, 5H), 7.02 (d, 1H), 6.75 (d, | |
| methylpyrimidin-2-amine | 1H), 6.11 (d, 2H), 4.31 (s, 2H), | |
| | 4.20 (d, 2H), 3.82 (d, 2H), 3.65 (s, | |
| | 2H), 1.98 (s, 3H) | |
| 1-{4-[7-(1H-benzimidazol-5- | ¹H NMR (400 MHz, DMSO-d₆) δ | MS (EI) for |
| yl)-2,3-dihydro-1,4- | 8.25 (s, 1H), 7.95 (d, 1H), 7.81 (s, | C₂₈H₂₈N₆O₂: |
| benzoxazepin-4(5H)-yl]-7- | 1H), 7.74-7.62 (m, 2H), 7.49 (d, | 481.3 (MH⁺) |
| (methyloxy)quinazolin-2-yl}- | 2H), 7.16 (s, 1H), 7.10-7.03 (m, | |
| N,N-dimethylmethanamine | 1H), 6.98 (d, 1H), 5.05 (s, 2H), | |
| | 4.45 (s, 2H), 4.20 (s, 2H), 3.87 (d, | |
| | 3H), 3.47 (s, 2H), 2.13 (d, 6H) | |
| N,N-dimethyl-1-{4-[7-(2- | ¹H NMR (400 MHz, DMSO-d₆) δ | MS (EI) for |
| methyl-1H-benzimidazol-5-yl)- | 7.97-7.91 (m, 1H), 7.67 (d, 2H), | C₂₉H₃₀N₆O₂: |
| 2,3-dihydro-1,4-benzoxazepin- | 7.52 (d, 1H), 7.47 (dd, 1H), | 495.2 (MH⁺) |
| 4(5H)-yl]-7- | 7.40 (dd, 1H), 7.16 (d, 1H), 7.06 (dd, | |
| (methyloxy)quinazolin-2- | 1H), 6.96 (dd, 1H), 5.04 (s, 2H), | |
| yl}methanamine | 4.45 (s, 2H), 4.19 (s, 2H), 3.90 (d, | |
| | 3H), 3.44 (s, 2H), 2.58 (s, 3H), | |
| | 2.24 (m, 6H) | |
| 5-[(4-fluorophenyl)methyl]-4- | ¹H NMR (400 MHz, DMSO-d₆) δ | MS (EI) for |
| methyl-6-[7-(2-methyl-1H- | 7.62 (s, 1H), 7.48 (m, 3H), | C29H27FN6O: |
| benzimidazol-5-yl)-2,3-dihydro- | 7.26-7.06 (m, 6H), 7.00 (d, 1H), | 495.3 (MH⁺) |
| 1,4-benzoxazepin-4(5H)- | 6.84 (s, 1H), 6.45 (s, 2H), 4.40 (s, 2H), | |
| yl]pyrimidin-2-amine | 4.22 (s, 2H), 3.85 (s, 2H), 3.71 (s, | |
| | 2H), 2.52 (s, 3H), 2.01 (s, 3H) | |
| 6-(4-{2- | ¹H NMR (400 MHz, d₆-DMSO): | MS (EI) for |
| [(dimethylamino)methyl]-6- | 8.38 (d, 1H), 7.88 (brs, 2H), | C₂₆H₃₁N₇OS: |
| methyl-5-(1- | 7.81 (d, 1H), 7.61 (d, 1H), 7.54 (dd, | 490 (MH⁺) |
| methylethyl)pyrimidin-4-yl}- | 1H), 7.03 (d, 1H), 4.46 (s, 2H), | |
| 2,3,4,5-tetrahydro-1,4- | 4.30 (brt, 2H), 3.68 (brt, 2H), | |
| benzoxazepin-7- | 3.35 (s, 2H), 3.25 (m, 1H), 2.47 (s, | |
| yl)[1,3]thiazolo[5,4-b]pyridin- | 3H), 2.10 (s, 6H), 1.30 (d, 6H) | |
| 2-amine | | |
| N-ethyl-N-({4-methyl-6-[7-(2- | ¹H NMR (400 MHz, DMSO-d6) δ | MS (EI) for |
| methyl-1H-benzimidazol-5-yl)- | 7.62 (s, 1H), 7.55 (br s, 1H), | C₃₀H₃₈N₆O: |
| 2,3-dihydro-1,4-benzoxazepin- | 7.47 (m, 3H), 7.35 (d, 1H), 6.99 (d, | 499.3 (MH⁺) |
| 4(5H)-yl]-5-(1- | 1H), 4.43 (s, 2H), 4.27 (br t, 2H), | |
| methylethyl)pyrimidin-2- | 3.68 (br s, 2H), 3.59 (s, 2H), | |
| yl}methyl)ethanamine | 2.47 (s, 3H), 1.91 (s, 3H), 1.32 (d, 6H), | |
| | 0.89 (t, 6H) | |
| {4-methyl-6-[7-(2-methyl-1H- | ¹H NMR (400 MHz, DMSO-d6 | MS (EI) for |
| benzimidazol-5-yl)-2,3-dihydro- | plus D₂O) δ 7.70 (s, 1H), 7.56 (m, | C₂₈H₃₁N₅O₃: |
| 1,4-benzoxazepin-4(5H)-yl]-5- | 3H), 7.38 (m, 1H), 7.06 (d, 1H), | 486.3 (MH⁺) |
| (1-methylethyl)pyrimidin-2- | 4.96 (s, 2H), 4.43 (s, 2H), 4.30 (br | |
| yl}methyl acetate | s, 2H), 3.68 (br s, 2H), | |
| | 3.31-3.20 (m, 1H), 2.46 (s, 3H), 2.08 (s, | |
| | 3H), 1.32 (d, 6H) | |
| {4-methyl-6-[7-(2-methyl-1H- | ¹H NMR (400 MHz, DMSO-d6 | MS (EI) for |
| benzimidazol-5-yl)-2,3-dihydro- | plus D₂O) δ 7.59 (m, 1H), | C₂₆H₂₉N₅O₂: |
| 1,4-benzoxazepin-4(5H)-yl]-5- | 7.54-7.45 (m, 3H), 7.36 (d, 1H), | 444.3 (MH⁺) |
| (1-methylethyl)pyrimidin-2- | 7.01 (d, 1H), 4.80 (br t, 0.2H), 4.43 (s, | |
| yl}methanol | 2H), 4.32 (s, 2H), 4.26 (br s, 2H), | |
| | 3.66 (br s, 2H), 3.25 (m, 1H), | |
| | 2.47 (s, 3H), 2.45 (s, 3H), 1.87 (s, 1H- | |
| | OAc peak), 1.29 (d, 6H) | |
| 6-(4-{2- | ¹H NMR (400 MHz, DMSO-d6) δ | MS (EI) for |
| [(dimethylamino)methyl]-5- | 8.37 (d, 1H), 7.87 (s, 2H), 7.81 (d, | C₂₅H₂₉N₇OS: |
| ethyl-6-methylpyrimidin-4-yl}- | 1H), 7.68 (d, 1H), 7.53 (dd, 1H), | 476.2 (MH⁺) |
| 2,3,4,5-tetrahydro-1,4- | 7.01 (d, 1H), 4.63 (s, 2H), 4.33 (br | |
| benzoxazepin-7- | t, 2H), 3.81 (br t, 2H), 2.62 (q, | |
| yl)[1,3]thiazolo[5,4-b]pyridin- | 2H), 2.35 (s, 3H), 2.10 (s, 6H), | |
| 2-amine | 1.88 (s, 2H-OAc peak), 1.13 (t, | |
| | 3H) | |
| N,N-dimethyl-1-{4-methyl-6- | ¹H NMR (400 MHz, DMSO-d6) δ | MS (EI) for |
| [7-(2-methyl-1H-benzimidazol- | 12.25 (s, 1H), 7.67 (m, 2H), | C₂₈H₃₄N₆O₂: |
| 6-yl)-2,3-dihydro-1,4- | 7.47 (dd, 2H), 7.39 (br d, 1H), 7.01 (d, | 487.3 (MH⁺) |
| benzoxazepin-4(5H)-yl]-5-[2- | 1H), 4.60 (s, 2H), 4.29 (br t, 2H), | |
| (methyloxy)ethyl]pyrimidin-2- | 3.79 (br t, 2H), 3.53 (t, 2H), | |
| yl}methanamine | 3.18 (s, 3H), 3.17 (s, 1H), 2.92 (t, 2H), | |
| | 2.39 (s, 3H), 2.13 (s, 6H) | |
| 6-(4-{2- | ¹H NMR (400 MHz, DMSO-d6) δ | MS (EI) for |
| [(dimethylamino)methyl]-6- | 8.36 (d, 1H), 7.89 (br s, 2H), | C₂₆H₃₁N₇O₂S: |
| methyl-5-[2- | 7.82 (d, 1H), 7.68 (d, 1H), 7.52 (dd, | 506.3 (MH⁺) |
| (methyloxy)ethyl]pyrimidin-4- | 1H), 7.00 (d, 1H), 4.59 (s, 2H), | |
| yl}-2,3,4,5-tetrahydro-1,4- | 4.29 (br t, 2H), 3.78 (br t, 2H), | |
| benzoxazepin-7- | 3.49 (t, 2H), 3.18 (s, 3H), 2.85 (t, | |
| yl)[1,3]thiazolo[5,4-b]pyridin- | 2H), 2.35 (s, 3H), 2.09 (s, 6H), | |
| 2-amine | 1.87 (s, 2H-OAc peak) | |
| 6-(4-{2- | ¹H NMR (400 MHz, DMSO-d6) δ | MS (EI) for |
| [(dimethylamino)methyl]-5,6- | 8.37 (d, 1H), 7.87 (s, 2H), 7.82 (d, | C₂₄H₂₇N₇OS: |
| dimethylpyrimidin-4-yl}- | 1H), 7.70 (d, 1H), 7.52 (dd, 1H), | 462.2 (MH⁺) |
| 2,3,4,5-tetrahydro-1,4- | 7.02 (d, 1H), 4.63 (s, 2H), 4.30 (br | |
| benzoxazepin-7- | t, 2H), 3.82 (br t, 2H), 3.33 (s, | |
| yl)[1,3]thiazolo[5,4-b]pyridin- | 2H), 2.30 (s, 3H), 2.16 (s, 3H), | |
| 2-amine | 2.10 (s, 6H), 1.90 (s, 3H-OAc | |
| | peak) | |
| 1-{4,5-dimethyl-6-[7-(2- | ¹H NMR (400 MHz, DMSO-d6) δ | MS (EI) for |
| methyl-1H-benzimidazol-6-yl)- | 7.61 (m, 2H), 7.53-7.43 (m, 2H), | C₂₆H₃₀N₆O: |
| 2,3-dihydro-1,4-benzoxazepin- | 7.36 (d, 1H), 7.00 (d, 1H), 4.61 (s, | 443.3 (MH⁺) |
| 4(5H)-yl]pyrimidin-2-yl}-N,N- | 2H), 4.29 (br t, 2H), 3.82 (br t, | |
| dimethylmethanamine | 2H), 3.39 (s, 2H), 2.31 (s, 3H), | |
| | 2.17 (s, 3H), 2.16 (s, 6H), 1.91 (s, | |
| | 3H-OAc Peak) | |
| {4-[7-(2- | ¹H NMR (400 MHz, d6-DMSO) δ | MS (ES) for |
| amino[1,3]thiazolo[5,4- | 8.39 (d, 1H), 7.88 (d, 1H), 7.85 (s, | C₂₇H₂₇N₇OS: |
| b]pyridin-6-yl)-2,3-dihydro-1,4- | 2H), 7.75 (s, 1H), 7.56 (d, 1H), | 498.2 (MH⁺) |
| benzoxazepin-4(5H)-yl]-6,6- | 7.05 (d, 1H), 4.70 (s, 2H), | |
| dimethyl-5,6,7,8- | 4.34 (m, 2H), 4.06 (s, 2H), 3.91 (m, | |
| tetrahydroquinazolin-2- | 2H), 2.70 (t, 2H), 2.46 (s, 2H), | |
| yl}acetonitrile | 1.59 (t, 2H), 0.85 (s, 6H) | |

### Biological Examples

Compounds of the Invention have activity for PI3K-alpha, mTOR, or for both. Compounds of this invention have been tested using the assays described in Biological Examples 1 and 3 and have been determined to be inhibitors of PI3K-alpha, mTOR, or for both.

Suitable in vitro assays for measuring PI3K, mTORc1, and mTORc2 activity and the inhibition thereof by compounds are known in the art. Biological Examples, Example 1, 2, and 3 describe in vitro assay for measuring PI3K and mTOR activity. Cell-based assays for measurement of in vitro efficacy in treatment of cancer are known in the art. Biological Examples, Example 5 and 6 describe assays to measure in vitro cell activity. Suitable in vivo models for cancer are known to those of ordinary skill in the art. Biological Examples 89, 10, 11, 12, 13, and 14 describe in vivo models for prostate adenocarcinoma, glioblastoma, lung carcinoma, and melanoma. Following the examples disclosed herein, as well as that disclosed in the art, a person of ordinary skill in the art can determine the PI3K-inhibitory and/or mTOR-inhibitory activity of a Compound of this invention.

Thus, compounds of Formula I are useful for treating diseases, particularly cancer in which activity against PI3K-alpha, mTOR, or both contributes to the pathology and/or symptomatology of the disease. For example, cancer in which activity against PI3K-alpha, mTOR, or both contributes to its pathology and/or symptomatology include breast cancer, mantle cell lymphoma, renal cell carcinoma, acute myelogenous leukemia, chronic myelogenous leukemia, NPM/ALK-transformed anaplastic large cell lymphoma, diffuse large B cell lymphoma, rhabdomyosarcoma, ovarian cancer, endometrial cancer, cervical cancer, non small cell lung carcinoma, small cell lung carcinoma, adenocarcinoma, colon cancer, rectal cancer, gastric carcinoma, hepatocellular carcinoma, melanoma, pancreatic cancer, prostate carcinoma, thyroid carcinoma, anaplastic large cell lymphoma, hemangioma, glioblastoma, or head and neck cancer.

### Biological Example 1

### mTOR/GbL/Raptor (mTORC1) ELISA Assay

The measurement of mTORC1 enzyme activity was performed in an ELISA assay format following the phosphorylation of 4E-BP1 protein. All experiments were performed in the 384-well format. Generally, 0.5 μL DMSO containing varying concentrations of the test Compound was mixed with 15 μL enzyme solution. Kinase reactions were initiated with the addition of 15 μL of substrates-containing solution. The assay conditions were as follows; 0.2 nM mTORC1, 10 μM ATP and 50 nM NHis-tagged 4E-BPI in 20 mM Hepes, pH 7.2, 1 mM DTT, 50 mM NaCl, 10 mM MnCl₂, 0.02 mg/mL BSA, 0.01% CHAPS, 50 mM β-glycerophosphate. Following an incubation of 120 minutes at ambient temperature, 20 μL of the reaction volume was transferred to a Ni-Chelate-coated 384-well plate. The binding step of the 4E-BP1 protein proceeded for 60 minutes, followed by washing 4 times each with 50 μL of Tris-buffered saline solution (TBS). Anti-phospho-4E-BP1 rabbit-IgG (20 μL, 1:5000) in 5% BSA-TBST (0.2% Tween-20 in TBS) was added and further incubated for 60 minutes. Incubation with a secondary HRP-tagged anti-IgG was similarly performed after washing off the primary antibody (4 washes of 50 μL). Following the final wash step with TBST, 20 μL of SuperSignal ELISA Femto (Pierce Biotechnology) was added and the luminescence measured using an EnVision plate reader.

As numbered in Table 1, Compounds 12, 13, 55, 65, 66, 70, 72-74, 78-80, 83, 89, 93, 94, 97, 102, 103, 124, 131, 142, 147, 148, 153, 170, 171, 174-178, 181, 183, 189, 192, 193, 213, 215, 217-221, 224-225, 229, 234, 236, 238, 248, 250, 252, 254, 255, 264-265, 290, 304, 308, 310-313, 321, 325, 339, 347-348, 352-354, 369, 371, 373, 375, 387, 397, 399, 401-402, 405-406, 409, 436, 439-440, 443, 447-450, 452, 474, 478-479, 483-484, 486, 488-491, 495, 500-502, 506-508, 514, 520, 537, 544-545, 548, 550, 552-555, 558, 564, 582-584, 586, 589, 590, 592-593, 596-601, 605, 607, 651, 660, 668-669, 676, 680, 684, 687, 689-690, and 692 have an IC₅₀ in this assay of less than or equal to 10 nM. As numbered in Table 1, Compounds 9, 11, 15, 16, 17, 21, 30, 37, 38, 68, 81, 82, 84, 86, 90-91, 95, 101, 113, 140-141, 143-146, 156, 158, 179-180, 182, 185-187, 194, 216, 226, 251, 256, 289, 291, 299-301, 305, 314-316, 318, 320, 322, 334, 351, 358, 360, 363-368, 370, 372, 376, 380, 382, 388-391, 396, 403, 407, 408, 411, 412, 414, 415, 418, 425, 426, 430-432, 434, 435, 437, 441, 442, 444-446, 451, 460, 462, 464, 465, 473, 482, 485, 487, 487, 492-494, 497, 503, 505, 510, 512, 517, 519, 526, 530, 540, 547, 556, 559, 566, 575-578, 585, 587-588, 591, 594-595, 602, 604, 606, 608, 614, 617, 619, 623, 626, 628, 636, 642, 665, 670, 674, 679, 682-683, and 686 have an IC₅₀ in this assay of greater than 10 nM but less than or equal to 50 nM. As numbered in Table 1, Compounds 1, 3, 8, 14, 19, 20, 22, 24-26, 28, 29, 32, 34-36, 39, 40, 43, 49, 58-61, 63, 64, 69, 76, 85, 98, 99, 105, 123, 149-151, 154, 159, 172, 188, 207, 208, 227, 228, 230, 233, 235, 239, 243, 244-246, 249, 253, 259, 260, 263, 268, 272, 275, 278, 281, 282, 283, 285, 287, 292, 295, 297, 298, 309, 317, 331-332, 336, 340, 346, 350, 355, 356, 362, 374, 377-379, 383-385, 392-395, 398, 400, 410, 413, 416-417, 422-424, 427, 438, 453, 455, 457-459, 461, 463, 466, 468, 469-472, 481,498-499, 509, 511, 513, 515-516, 518, 523, 536, 538-539, 542, 546, 560, 565, 567-571, 580, 603, 612, 616-617, 620, 625, 627, 633, 641, 650, 654, 657, 662-663, 667, 675, 681, and 685 have an IC₅₀ in this assay of greater than 50 nM but less than or equal to 250 nM. As numbered in Table 1, Compounds 4, 5, 10, 18, 23, 27, 31, 33, 41, 42, 45, 46, 50, 51, 53-54, 56, 62, 67, 75, 87, 92, 96, 100, 108-109, 112, 114, 126-127, 129, 130, 152, 155, 157, 160, 162, 164, 166, 168-169, 173, 184, 190-191, 199, 202, 210, 212, 214, 222, 237, 241, 247, 257, 258, 261, 266-267, 269-271, 273, 276, 279, 280, 284, 302-303, 306-307, 323-324, 326-327, 330, 337, 341-342, 344-345, 349, 357, 359, 381, 386, 419-421, 429, 454, 476-477, 480, 496, 504, 521-522, 525, 527, 529, 531-532, 535, 551, 557, 561-563, 573, 579, 610-611, 613, 618, 621-622, 624, 629, 632, 634-635, 637, 639-640, 643, 647-649, 652-653, 655-656, 659, 661, 664, 671, 673, 678, and 688 have an IC₅₀ in this assay of greater than 250 nM but less than or equal to 1000 nM. As numbered in Table i, Compounds 6, 7, 44, 48, 52, 71, 77, 88, 106, 111, 116, 133, 135-137, 161, 195, 197, 200-201, 203,205-206, 211, 231, 277, 335, 343, 524, 534, 543, 574, 581, and 691 have an IC₅₀ in this assay of greater than 1000 nM but less than 2000 nM. As numbered in Table 1, Compounds 2, 47, 57, 104, 107, 110, 115, 117-122, 128, 132, 134, 138, 163, 165, 167, 196, 198, 204, 209, 223, 232, 240, 242, 262, 274, 286, 288, 293-294, 296, 328-329, 333, 338, 361, 428, 433, 456, 475, 533, 541, 572, 609, 630, 631, 638, 644-646, 658, 666, 672, and 677 have an IC₅₀ in this assay of greater than or equal to 2,000 nM or are not active under the conditions the assay was run.

### Biological Example 2

### Immune-Complex mTORC2 Kinase (mTORC2 IP-Kinase) Assay

HeLa (ATCC) cells are grown in suspension culture and lysed in ice-cold lysis buffer containing 40 mM HEPES pH 7.5, 120 mM NaCl, 1 mM EDTA, 10 mM sodium pyrophosphate, 10 mM β-glycerophosphate, 10 mM NaF, 10 mM NaN₃, one tablet of protease inhibitors (Complete-Mini, EDTA-free, Roche), 0.3% cholamidopropyldimethylammoniopropanesulfonate (CHAPS), 1 mM AEBSF, 0.5 mM benzamidine HCl, 20 μg/mL heparin, and 1.5 mM Na₃VO₄. The mTORC2 complex is immunoprecipitated with anti-RICTOR antibody for 2 h. The immune complexes are immobilized on Protein A sepharose (GE Healthcare, 17-5280-01), washed sequentially 3 times with wash buffer (40 mM HEPES pH 7.5, 120 mM NaCl, 10 mM β-glycerophosphate, 0.3% CHAPS, 1 mM AEBSF, 20 μg/mL heparin, 1.5 mM Na₃VO₄, and Complete-Mini, EDTA-free) and resuspended in kinase buffer (40 mM HEPES, pH 7.5, 120 mM NaCl, 0.3% CHAPS, 20 g/mL heparin, 4 mM MgCl₂, 4 mM MnCl₂, 10% Glycerol, and 10 mM DTT). The immune complexes (equivalent to 1×10⁷ cells) are pre-incubated at 37° C. with a test Compound or 0.6% DMSO for 5 min, and then subjected to a kinase reaction for 8 min in a final volume of 33 μL (including 5 μL bed volume) containing kinase buffer, 50 μM ATP, and 0.75 μg full length dephosphorylated AKT1. Kinase reactions are terminated by addition of 11 μL 4×SDS sample buffer containing 20% β-mercaptoethanol and resolved in a 10% Tris Glycine gels. The gels are transferred onto PVDF membrane at 50 V for 20 h at 4° C. The membranes are blocked in 5% non-fat milk in TBST for 1 h and incubated overnight at 4° C. with 1/1000 dilution of rabbit anti-pAKT (S473) (Cell Signaling Technology, 4060) in 3% BSA/TBST. The membranes are washed 3 times in TBST and incubated for 1 h with a 1/10000 dilution of secondary goat anti-rabbit HRP antibody (Cell Signaling Technology, 2125) in 5% non-fat milk/TBST. The signal is detected using Amersham ECL-plus. The scanned data are analyzed using ImageQuant software. IC₅₀ for the test Compound is determined relative to DMSO treated sample using XLfit4 software.

### Biological Example 3

### PI3K Biochemical Assays

PI3Kα activity is measured as the percent of ATP consumed following the kinase reaction using luciferase-luciferin-coupled chemiluminescence. Reactions were conducted in 384-well white, medium binding microtiter plates (Greiner). Kinase reactions were initiated by combining test compounds, ATP, substrate (PIP2), and kinase in a 20 μL volume in a buffer solution. The standard PI3Kalpha assay buffer is composed 50 mM Tris, pH 7.5, 1 mM EGTA, 10 mM MgCl₂, 1 mM DTT and 0.03% CHAPS. The standard assay concentrations for enzyme, ATP, and substrate are 1.5 nM, 1 μM, and 10 μM, respectively. The reaction mixture was incubated at ambient temperature for approximately 2 h. Following the kinase reaction, a 10 μL aliquot of luciferase-luciferin mix (Promega Kinase-Glo) was added and the chemiluminescence signal measured using a Victor2 plate reader (Perkin Elmer). Total ATP consumption was limited to 40-60% and IC₅₀ values of control compounds correlate well with literature references. Substituting PI3Kα with PI3Kβ, PI3Kγ, or PI3Kδ, the inhibitory activity of the compounds for the other isoforms of I3K were measured.

As numbered in Table 1, Compounds 9, 94, 103, 113, 119, 124, 131, 175-183, 185-188, 192, 208, 217-218, 237, 241, 246-247, 250, 256, 264, 268, 279-282, 285, 289-292, 295, 301, 303, 308, 310, 315, 319, 321-322, 325, 332, 334, 339, 346-348, 350-355, 364, 366, 368, 370-371, 375, 397, 399, 412, 414, 441, 454, 462, 487-488, 499-501, 506, 526, 527, 537, 539-540, 542, 565, 568, 577-580, 582-583, 586, 589, 596, 600-601, 605-608, 613-616, 623, 628, 641-643, 651, 654, 657-664, 670, 671, 674, 680, and 689-692 have an IC₅₀ for PI3Kα in this assay of less than or equal to 10 nM. As numbered in Table 1, Compounds 1, 3, 4, 13-15, 18, 19, 21, 26, 30, 37-39, 55, 63, 65, 68, 70, 72-74, 79-81, 83, 89-91, 93, 97-98, 101-102, 108-109, 111-112, 123, 125-126, 142, 145-147, 157, 160, 164, 168, 170-171, 184, 189, 190-191, 193, 194, 199, 207, 210, 213, 215, 220-221, 224, 227-228, 234-235, 238, 248, 251, 255, 258-261, 263, 265, 271, 278, 283, 287, 293, 299-300, 302, 304-305, 309, 311-314, 316, 318, 320, 323, 331, 333, 335, 340-341, 343, 344-345, 349, 356, 359-360, 369, 372-373, 377-378, 380-381, 387-388, 390-392, 396, 401-402, 405, 407-409, 413, 415-416, 424, 426-427, 430-432, 434, 436-437, 439-440, 442-443, 445-446, 447, 449-452, 459-461, 463-464, 474, 478-479,482-483, 489-492, 495, 497-498, 502, 505, 507-508, 510, 512, 519-520, 525, 530-531, 545, 546-548, 550, 552-556, 558, 560, 564, 567, 575, 584-585, 587-588, 590, 592-595, 597-598, 599, 602-603, 610, 612, 617-619, 633, 636-637, 639-640, 649, 652-653, 656, 667-669, 672-673, 675-676, 678-679, 682-685, and 687 have an IC₅₀ for PI3Kα in this assay of greater than 10 nM but less than or equal to 50 nM. As numbered in Table 1, Compounds 5, 8, 11, 12, 16, 17, 20, 22, 23, 25, 27, 29, 36, 40, 42, 43, 46, 48, 51, 56-58, 62, 64, 66, 69, 76-78, 82, 84, 86, 92, 95, 99-100, 105, 120-122, 132, 140-141, 144, 148, 153, 156, 158-159, 166, 174, 197, 214, 216, 219, 225-226, 229-230, 233, 236, 239, 242, 245, 249, 252, 254, 257, 267, 269, 270, 272-273, 275, 286, 297-298, 317, 327, 330, 342, 358, 361-363, 365, 367, 374, 376, 382-386, 389, 393-395, 398, 400, 403, 406, 410-411, 417-423, 425, 429, 435, 438, 444, 448, 453, 455, 458, 465-466, 468-470, 473,480-481, 485-486, 493-494, 496, 503, 509, 511, 513-518, 521-522, 529, 532, 534, 538, 544, 551, 559, 563, 566, 570-571, 576, 591, 604, 620, 625, 626-627, 632, 634, 648, 665-666, 677, 681, and 686 have an IC₅₀ for PI3Kα in this assay of greater than 50 nM but less than or equal to 250 nM. As numbered in Table 1, Compounds 2, 10, 24, 28, 31-33, 44, 45, 47, 49, 50, 54, 59-61, 96, 107, 110, 116-117, 129, 137-138, 143, 149-152, 154-155, 161, 162-163, 165, 167, 169, 172, 195-196, 209, 211, 222, 240, 243-244, 253, 266, 274, 276-277, 284, 296, 306-307, 324, 336-338, 357, 379, 428, 456-457, 471-472, 476-477, 504, 523-524, 533, 536, 541, 543, 569, 573-574, 609, 621, 624, 635, 650, and 655 have an IC₅₀ for PI3Kα in this assay of greater than 250 nM but less than or equal to 1000 nM. As numbered in Table 1, Compounds 41, 67, 71, 75, 85, 106, 115, 118, 130, 139, 204-205, 232, 288, 294, 326, 328-329, 433, 535, 561-562, 572, 611, 622, 645, 646, and 688 have an IC₅₀ for PI3Kα in this assay of greater than 1000 nM but less than 2000 nM. As numbered in Table 1, Compounds 6, 7, 52-53, 87-88, 104, 127-128, 133-135, 173, 198, 200-203, 206, 212, 223, 231, 262, 475, 557, 581, 629-631, 638, 644, and 647 have an IC₅₀ for PI3Kα in this assay of greater than or equal to 2,000 nM or are not active under the conditions the assay was run.

### Embodiments 1

In one embodiment the invention comprises a compound of the invention having a PI3K-alpha-inhibitory activity of about 0.5 μM or less and is inactive for mTOR (when tested at a concentration of 2.0 μM or greater) or is selective for PI3K-alpha over mTOR by about 5-fold or greater, about 7-fold or greater, or about 10-fold or greater. In another embodiment, the invention comprises a compound of the invention having a PI3K-alpha-inhibitory activity of about 0.35 μM or less and is inactive for mTOR (when tested at a concentration of 2.0 μM or greater) or is selective for PI3K-alpha over mTOR by about 5-fold or greater, about 7-fold or greater, or about 10-fold or greater. In another embodiment, the invention comprises a compound of the invention having a PI3K-alpha-inhibitory activity of about 0.25 μM or less and is inactive for mTOR (when tested at a concentration of 2.0 μM or greater) or is selective for PI3K-alpha over mTOR by about 5-fold or greater, about 7-fold or greater, or about 10-fold or greater. In another embodiment the compounds of the invention have an PI3K-alpha-inhibitory activity of about 0.1 μM or less and is inactive for mTOR (when tested at a concentration of 2.0 μM or greater) or is selective for PI3K-alpha over mTOR by about 5-fold or greater, about 7-fold or greater, or about 10-fold or greater. In another embodiment the invention comprises a compound of the invention having an PI3K-alpha-inhibitory activity of about 0.05 μM or less and is selective for PI3K-alpha over mTOR by about 5-fold or greater, about 7-fold or greater, or about 10-fold or greater.

### Embodiments 2

In one embodiment the invention comprises a compound of the invention having a PI3K-alpha-inhibitory activity of about 2.0 μM or less and an mTOR-inhibitory activity of about 2.0 μM or less and the selectivity for one of the targets over the other does not exceed 3-fold. In another embodiment the invention comprises a compound of the invention having a PI3K-alpha-inhibitory activity of about 1.0 μM or less and an mTOR-inhibitory activity of about 1.0 μM or less and the selectivity for one of the targets over the other does not exceed 3-fold. In another embodiment the invention comprises a compound of the invention having a PI3K-alpha-inhibitory activity of about 0.5 μM or less and an mTOR-inhibitory activity of about 0.5 μM or less and the selectivity for one of the targets over the other does not exceed 3-fold. In another embodiment the invention comprises a compound of the invention having a PI3K-alpha-inhibitory activity of about 0.3 μM or less and an mTOR-inhibitory activity of about 0.3 μM or less and the selectivity for one of the targets over the other does not exceed 3-fold. In another embodiment the invention comprises a compound of the invention having a PI3K-alpha-inhibitory activity of about 0.2 μM or less and an mTOR-inhibitory activity of about 0.2 μM or less and the selectivity for one of the targets over the other does not exceed 2-fold. In another embodiment the invention comprises a compound of the invention having a PI3K-alpha-inhibitory activity of about 0.15 μM or less and an mTOR-inhibitory activity of about 0.15 μM or less and the selectivity for one of the targets over the other does not exceed 2-fold. In another embodiment the invention comprises a compound of the invention having a PI3K-alpha-inhibitory activity of about 0.1 μM or less and an mTOR-inhibitory activity of about 0.1 μM or less. In another embodiment the invention comprises a compound of the invention having a PI3K-alpha-inhibitory activity of about 0.05 μM or less and an mTOR-inhibitory activity of about 0.05 μM or less. In another embodiment the invention comprises a compound of the invention have a PI3K-alpha-inhibitory activity of about 0.02 μM or less and an mTOR-inhibitory activity of about 0.02 μM or less.

In another embodiment the invention comprises a compound of the invention have a PI3K-alpha-inhibitory activity of about 0.01 μM or less and an mTOR-inhibitory activity of about 0.01 μM or less. As shown in Table 3, Compounds 698, 696 and 699 (PI3Kα selective compounds) were shown to be specific for PI3Kα isoforms, but not PI3Kβ, PI3Kδ, PI3Kγ and mTOR. The dual PI3Kα/mTOR selective inhibitors were similarly not active against PI3Kβ, PI3Kδ, PI3Kγ, but were active in inhibiting the activity of mTOR. The PI3Kβ active compound TGX-221 was active against PI3Kβ mediated activity. See Table 3.

TABLE 3
PI3K biochemical IC₅₀ values (nM)
Table 3. PI3K biochemical IC₅₀ values (nM)

| Compound PI3Kα | | PI3Kα | PI3Kα | | | | |
|---|---|---|---|---|---|---|---|
| (Table 1) | PI3Kα | E545K | H1047R | PI3Kβ | P13Kδ | PI3Kγ | mTOR |
| 698 | 3 | 6 | 7 | 1559 | 162 | 159 | 120 |
| 696 | 3 | 9 | 5 | 1314 | 149 | 1800 | 381 |
| 699 | 6 | 5 | 6 | 1743 | 440 | 158 | 252 |
| 693 | 2 | 5 | 3 | >3000 | 575 | 1118 | 6 |
| 694 | 4 | 6 | 5 | >3000 | 831 | 1851 | 16 |
| TGX-221 | 1775 | nd | nd | 16 | 142 | 1678 | 10856 |

PIP2 used as substrate for PI3K Class 1 isoforms
4EBP1 used as substrate for mTOR/GbL/raptor kinase

In some embodiments, a series of PI3K inhibitors with varying isozyme selectivity were profiled for inhibition of IGF1-mediated AKT(T308) phosophorylation in MCF-7 cells (PI3Kα-E545K mutant) and T-47D cells (PI3Kα-H1047R mutant)

TABLE 4
Cell Line Sources and Genetic Backgrounds

| | | PIK3CA | PTEN | KRAS | |
|---|---|---|---|---|---|
| Cell Line | Source | Status | Status | Status | Other |
| MCF-7 | ATCC/HTB-22 | E545K het | positive | wild type | |
| NCI-H460 | ATCC/HTB-177 | E545K het | positive | Q61H hom | |
| T-47D | ATCC/HTB-133 | H1047R het | positive | wild type | |
| HCT 116 | ATCC/CCL-247 | H1047R het | positive | G13D het | |
| MDA-MB-453 | ATCC/HTB-131 | H1047R het | positive | wild type | HER2 over-expression |
| BT-20 | ATCC/HTB-19 | H1047R het | positive | wild type | PIK3CA-P539R + H1047R |
| LS 174T | ATCC/CL-188 | H1047R het | positive | G12D het | |
| PC-3 | ATCC/CRL-1435 | wild type | negative | wild type | |
| MDA-MB-468 | ATCC/HTB-132 | wild type | negative | wild type | EGFR over-expression |
| SK-BR-3 | ATCC/HTB-30 | wild type | positive | wild type | HER2 over-expression |
| MDA-MB-231T | Georgetown U | wild type | positive | G13D het | |
| A549 | ATCC/CCL-185 | wild type | positive | G12S hom | |

TABLE 5
Cell Line Seeding Densities for ELISA and Proliferation Assays

| | | ELISA | | Proliferation |
|---|---|---|---|---|
| | | Seeding | ELISA | Seeding |
| | | Density | Growth Factor | Density |
| Cell Line | Growth Medium | cells/well | Stimulation | cells/well |
| MCF-7 | DMEM + 10% FBS + P/S + NEAA | 24,000 | IGF1 | Update |
| | | | | Column |
| NCI-H460 | DMEM + 10% FBS + P/S + NEAA | 12,000 | IGF1 | |
| T-47D | RPMI 1640 + 10% FBS + P/S + 0.2 U/mL | 30,000 | IGF1 | |
| | insulin | | | |
| HCT 116 | Leibovitz's L-15 + 10% FBS + P/S (C0₂ free) | 30,000 | IGF1 | |
| MDA-MB-453 | DMEM + 10% FBS + P/S + NEAA | 40,000 | heregulin | |
| BT-20 | EMEM + 10% FBS + P/S + NEAA | 20,000 | heregulin | |
| LS 174T | DMEM + 10% FBS + P/S + NEAA | N/A | N/A | |
| PC-3 | DMEM + 10% FBS + P/S + NEAA | 16,000 | IGF1 | |
| MDA-MB-468 | DMEM/F-12 + 10% FBS + P/S + NEAA | N/A | N/A | |
| SK-BR-3 | DMEM/F-12 + 10% FBS + P/S + NEAA | 25,000 | heregulin | |
| MDA-MB- | DMEM + 10% FBS + P/S + NEAA | N/A | N/A | |
| 231T | | | | |
| A549 | DMEM + 10% FBS + P/S + NEAA | 12,000 | IGF1 | N/A |

N/A = not applicable
NEAA = 1% non-essential amino acids,
P/S = 1% penicillin/streptomycin
IGF1 = 300 ng/mL long R3 IGF1 (Sigma, I1271) for 10 min (except HCT 116 for 20 min)
Heregulin = 100 ng/mL recombinant human HRG1-beta1 EGF domain (R&D Systems #, 396-HB) for 15 min
EGF = 20 ng/mL EGF (US Biologicals, E3374-07A) for 10 min

### Biological Example 4

### pS6 (S240/244) ELISA Assay

MCF-7 cells (ATCC) cells were seeded at 24000 cells per well in 96-well plates (Corning, 3904) in DMEM (Cellgro) containing 10% FBS (Cellgro), 1% NEAA (Cellgro) and 1% penicillin-streptomycin (Cellgro). Cells were incubated at 37° C., 5% CO2 for 48 h, and the growth medium was replaced with serum-free DMEM or in medium containing 0.4% BSA. Serial dilutions of the test Compound in 0.3% DMSO (vehicle) were added to the cells and incubated for 3 h. To fix the cells, medium was removed and 100 μL/well of 4% formaldehyde (Sigma Aldrich, F8775) in TBS (20 mM Tris, 500 mM NaCl) was added to each well at RT for 30 min. Cells were washed 4 times with 200 μL TBS containing 0.1% Triton X-100 (Sigma, catalog #T9284). Plates were blocked with 100 μL Odyssey blocking buffer (Li-Cor Biosciences, 927-40000) for 1 h at RT. Anti-pS6 (S240/244) antibody (Cell Signaling Technology, 2215) and anti-total-S6 antibody (R&D systems, MAB5436) were diluted 1:400 in Odyssey blocking buffer, and 50 μL of the antibody solution containing both antibodies was added to one plate to detect pS6 and total S6. After incubation overnight at 4° C., plates were washed 4 times with 200 μL TBS containing 0.1% Tween20 (Bio-Rad, catalog #170-6351) (TBST). Goat anti-rabbit and Goat anti-mouse secondary antibody (Li-Cor Biosciences, catalog #926-32221 and 926-32210) conjugated to IRDye were diluted 1:400 in Odyssey blocking buffer containing 0.1% Tween20. 50 μL of antibody solution containing both antibodies was added to each well and incubated for 1 h at RT. Plates were washed 3 times with 200 μL TBST and 2 times with 200 μL TBS. Fluorescence was read on an Odyssey plate reader. IC50 values were determined based on the ratio of pS6 to total S6 signal for Compound treated wells, normalized to the DMSO-treated control wells.

In one embodiment, the Compounds of the Invention tested in this assay in MCF-7 cells had an inhibitory activity of 1.5 μM or less. In another embodiment, the Compounds of the Invention tested in this assay in MCF-7 cells had an inhibitory activity of 1.0 μM or less. In another embodiment, the Compounds of the Invention tested in this assay in MCF-7 cells had an inhibitory activity of 0.5 μM or less. In one embodiment, the Compounds of the Invention tested in this assay in MCF-7 cells had an inhibitory activity of 0.3 μM or less. In one embodiment, the Compounds of the Invention tested in this assay in MCF-7 cells had an inhibitory activity of 0.1 μM or less. In one embodiment, the Compounds of the Invention tested in this assay in MCF-7 cells had an inhibitory activity of 0.03 μM or less.

In one embodiment, the Compound of the Invention tested in this assay in PC-3 cells had an inhibitory activity of about 1.7 μM or less. In another embodiment, the Compound of the Invention tested in this assay in PC-3 cells had an inhibitory activity of about 0.55 μM or less. In another embodiment, the Compound of the Invention tested in this assay in PC-3 cells had an inhibitory activity of about 0.55 μM or less. In another embodiment, the Compound of the Invention tested in this assay in PC-3 cells had an inhibitory activity of about 0.3 μM or less. In another embodiment, the Compound of the Invention tested in this assay in PC-3 cells had an inhibitory activity of about 0.1 μM or less. In another embodiment, the Compound of the Invention tested in this assay in PC-3 cells had an inhibitory activity of about 0.05 μM or less.

In another embodiment, cells were seeded onto 96-well plates (Corning, 3904) in their respective growth media at the densities listed in Table 5. Cells were incubated at 37° C., 5% CO₂ for 48 h. Compounds were serially diluted in DMSO and subsequently diluted in serum-free DMEM. Test compounds were added to cells in serum-free DMEM at a final concentration of 0.3% DMSO and incubated for 3 h. Cells were then stimulated with growth factors as listed in Table 5. To fix the cells, medium was removed and 50 μL/well of 4% formaldehyde (Sigma, F8775) in high-salt TBS (20 mM Tris, pH 7.4; 500 mM NaCl) was added to each well at RT for 30 min. Cells were washed 3 times with 200 μL high salt TBST and blocked with Odyssey blocking agent (Li Cor Biosciences #, 927 40000) for 1 h. Anti-phospho-RPS6(S240/244) antibody (1:400 dilution factor, Cell Signaling Technology #, 2215L) and anti-total-RPS6 antibody (1:2000 dilution factor, Santa Cruz Biotech #, sc 74576) were diluted in Odyssey blocking solution and 50 μL added per well. After incubation overnight at 4° C., plates were washed 4 times with 200 μL high salt TBST. Goat anti-rabbit IRDye 800CW- and goat anti-mouse IRDye 680-conjugated secondary antibodies (Li-Cor Biosciences #, 926 32211 and #, 926 32220, respectively) diluted 1:400 in Odyssey blocking buffer containing 0.1% Tween 20 were added to each well for 2 h at RT. Plates were washed 3 times with 200 μL high salt TBST and 3 times with 200 μL high salt TBS and then read on a Li-Cor Odyssey Infrared Imager with In-cell Western plug-in. Integrated intensities for phospho-RPS6(S240/244) were normalized to the signal for total RPS6 and IC₅₀ values were determined relative to the DMSO treated control.

### Biological Example 5

### pAKT (T308) ELISA Assay

MCF-7 cells (ATCC) cells were seeded at 24000 cells per well in 96-well plates (Corning, 3904) in DMEM (Cellgro) containing 10% FBS (Cellgro), 1% NEAA (Cellgro) and 1% penicillin-streptomycin (Cellgro). Cells were incubated at 37° C., 5% CO2 for 48 h, and the growth medium was replaced with serum-free DMEM or in medium containing 0.4% BSA. Serial dilutions of the test Compound in 0.3% DMSO (vehicle) were added to the cells and incubated for 3 h. At the end of the incubation period, cells were stimulated for 10 minutes by the addition of L-IGF (Sigma, I-1271) at a final concentration of 100 ng/ml. Afterwards, media was discarded from cell plates and 110 μl/well of cold lysis buffer (see table below) were added. Cell plates were incubated on ice and then put on shaker in 4° C. cold room for 1 h. Two capture plates (Thermo Scientific, Reacti-bind plate, 15042) were prepared for each cell plate by pre-coating with capture Akt antibody from the two sandwich ELISA antibody pairs used (Cell Signaling Technology 7142 and 7144). The Akt capture antibodies were diluted 1:100 in PBS and 100 μl of diluted capture antibody was added per well. Capture plates were incubated at 4 C overnight. Prior to use, capture plates were washed 3 times in TBS containing 0.1% Tween20 (Bio-Rad, 170-6351) (TBST) and blocked in blocking buffer (Thermo Scientific, Starting Block T20, 37543) for 1-2 h at room temperature. After 1 h of cell lysis, 85 μl of cell lysate/well was transferred to the capture plate for detection of pAkt(T308). 15 μl of cell lysate was transferred from same well to the second capture plate for detection of total Akt1. After incubation overnight at 4° C., plates were washed 3 times with 200 μL TBST. Primary antibodies, diluted 1:100 in blocking buffer, were added to the corresponding capture plates for pAkt(T308) (Cell Signaling Technology, 7144) and total Akt1 (Cell Signaling Technology, 7142) detection and incubated at room temperature for 1 h. Plates were washed 3 times with 200 μL of TBST. Goat anti-mouse secondary antibody (Cell Signaling Technology, 7076) conjugated to HRP was diluted 1:1000 in blocking buffer and 100 μl were added to each well and incubated for 30 minutes at room temperature. Plates were then washed 3 times with 200 μL of TBST. 100 μL of SuperSignal ELISA Femto stable peroxidase solution (Thermo Scientific, 37075) was added to each well. After 1 minute incubation, chemiluminescence was read on a Wallac Victor2 1420 multilabel counter. IC50 values were determined based on the ratio of pAkt(T308) to total Akt1 signal for Compound treated wells, normalized to the DMSO-treated control wells.

| | Stock | Final | /10 mL | |
|---|---|---|---|---|
| Water | | | 6 | mL |
| Complete Protease | | | 1 mini- | |
| Inhibitors (Roche 1 836 | | | tablet | |
| 170) | | | | |
| 5x RIPA | 5x | 1x | 2 | mL |
| NaF | 200 mM | 1 mM | 50 | μL |
| B-glycerophosphate | 100 mM | 20 mM | 1.8 | mL |
| Phosphatase Inhibitor I | 100x | 1x | 100 | μL |
| (Sigma P2850) | | | | |
| Na orthovanadate | 200 mM | 1 mM | 50 | μL |
| EDTA, pH 8 | 500 mM | 1 mM | 20 | μL |

In one embodiment, the Compounds of the Invention tested in this assay in PC-3 cells had an inhibitory activity of about 2.0 μM or less. In another embodiment, the Compounds of the Invention tested in this assay in PC-3 cells had an inhibitory activity of about 1.0 μM or less. In another embodiment, the Compounds of the Invention tested in this assay in PC-3 cells had an inhibitory activity of about 0.3 μM or less. In another embodiment, the Compounds of the Invention tested in this assay in PC-3 cells had an inhibitory activity of about 0.2 μM or less.

In one embodiment, the Compounds of the Invention tested in this assay in MCF-7 cells had an inhibitory activity of about 3.0 μM or less. In another embodiment, the Compounds of the Invention tested in this assay in MCF-7 cells had an inhibitory activity of about 3.0 μM or less. In another embodiment, the Compounds of the Invention tested in this assay in MCF-7 cells had an inhibitory activity of about 1.5 μM or less. In another embodiment, the Compounds of the Invention tested in this assay in MCF-7 cells had an inhibitory activity of about 0.75 μM or less. In another embodiment, the Compounds of the Invention tested in this assay in MCF-7 cells had an inhibitory activity of about 0.5 μM or less. In another embodiment, the Compounds of the Invention tested in this assay in MCF-7 cells had an inhibitory activity of about 0.25 μM or less. In another embodiment, the Compounds of the Invention tested in this assay in MCF-7 cells had an inhibitory activity of about 0.1 μM or less.

In another embodiment, the pAKT ELISA Assay was performed by seeding cells onto 96-well plates (Corning, 3904) in their respective growth media at the densities listed in Table 5. Cells were incubated at 37° C., 5% CO₂ for 48 h (except MDA-MB-453 cells which were cultured in the absence of CO₂). Compounds were serially diluted in DMSO and subsequently diluted in serum-free DMEM. Test compounds were added to cells in serum-free DMEM at a final concentration of 0.3% DMSO and incubated for 3 h. Cells were then stimulated with growth factors as listed in Tables 3 and 4. Cells were lysed with 130 μL of ice-cold RIPA lysis buffer (50 mM Tris-HCl, pH 7.5; 0.5% sodium deoxycholic acid; 1% Triton X-100; 0.1% SDS; 150 mM NaCl) with protease and phosphatase inhibitors (1 mM EDTA, 1 mM NaF, 20 mM β-glycerophosphate, 1 mM Na-orthovanadate, Complete-Mini EDTA-free, Roche, 11836170001, and Phosphatase Inhibitor Cocktail I, Sigma, P2850), directly in 96-well plates. Cells were lysed on ice for 60-120 min at 4° C. The supernatants were added directly to capture ELISA plates previously pre-coated with rabbit anti-AKT capture antibody. ELISA assays for pAKT(T308) and total AKT were performed with the pAKT(T308) ELISA kit (Cell Signaling Technology, 7144) and AKT1 ELISA kit (Cell Signaling Technology, 7142). For the pAKT(T308) ELISA, 85 μL of cell lysate was added to each well. For the total AKT1 ELISA, 15 μL of cell lysate and 85 μL of Starting Block buffer (Pierce, 37543) were added to each well. The plates were incubated overnight at 4° C. with gentle shaking, then washed 3 times with 200 μL of TBST (20 mM Tris, pH 7.4; 150 mM NaCl; 0.1% Tween 20) for 30 s each, and incubated for 1 h with 100 μL of detection antibody (mouse anti-pAKT(T308) antibody or mouse anti-AKT1 antibody). After 3 washes with TBST, the plates were incubated with a 1:1000 dilution of a horseradish peroxidase-labeled anti-mouse IgG (Cell Signaling Technology, 7076) in 100 μL of Starting Block buffer for 0.5 h. After 3 washes with TBST, 50 μL of SuperSignal ELISA Femto substrate solution (Thermo Scientific, 37075A) and 50 μL of SuperSignal ELISA enhancer solution mixture (Thermo Scientific, 37075B) were added to each well and the plates were incubated for approximately 2 min and protected from light. The wells were measured for luminescence using a Wallac plate reader at wavelength of 560 nm. After normalization of pAKT signal to total AKT1 signal, IC₅₀ values were determined relative to the DMSO-treated control.

Results of PI3Kα inhibitor activated vs. PTE-null cellular models are provided in Table 6. PI3K-α selective compounds inhibit IGF1-mediated AKT phosphorylation with similar potencies in MCF-7 (PI3K-α E545K) and PC3 (PTEN null) cellular models. Dual PI3K-α/mTOR selective inhibitors are more potent that PI3K-α-selective compounds in both models. PI3K-α selective compounds show 5 to 10 fold greater potency in inhibiting pAKT (T308) in H1048R-mutated vs. E545K-mutated cell lines.

TABLE 6
PI3Kα inhibitor activity in PI3K-activated vs.
PTEN-null cellular models
Table 6. PI3Kα inhibitor activity in PI3K-activated vs.
PTEN-null cellular models

| | | | pAKT (T308) IC₅₀ (nM) | | | |
|---|---|---|---|---|---|---|
| | | | MCF-7 | T-47D | | |
| | Biochemical | | PI3Kα | PI3Kα | PC# | |
| Compound | IC₅₀ (nM) | | E545K | H1047R | PTEN-null | |
| (Table 1) | PI3Kα | mTOR | IGF1 | IGF1 | IGF1 | LPA |
| 698 | 3 | 120 | 2243 | 258 | 2871 | 4539 |
| 696 | 3 | 381 | 1649 | 213 | 1685 | 1865 |
| 699 | 6 | 252 | 2399 | 207 | 2120 | 3805 |
| 693 | 2 | 6 | 120 | 73 | nd | nd |
| 694 | 4 | 16 | 135 | 102 | 231 | 571 |
| TGX-221 | 1775 | 10856 | 1764 | 27908 | 11226 | 16 |

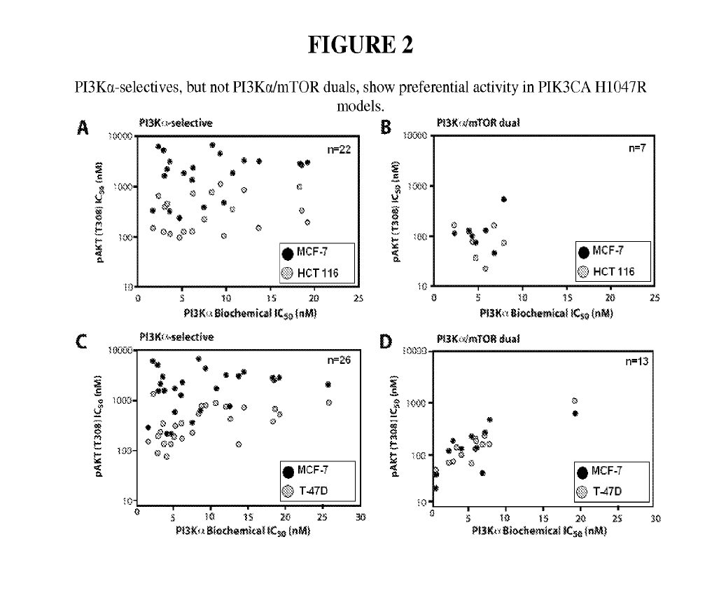

In order to determine if PI3Kα mutation status correlated with increased sensitivity of AKT(T308) to isozyme selective inhibitors, a representative PI3Kα/mTOR dual and a representative PI3Kα selective compound were tested for inhibition of growth factor mediated AKT(T308) phosphorylation in a E545K line (MCF-7), and two additional H1047R lines (HCT 116, and T-47D). pAKT(T308) IC₅₀ values for PI3Kα inhibitors were significantly lower in the two kinase domain mutant lines suggesting that the H1047R mutation may sensitize tumor cells to inhibition of PI3K signaling by PI3Kα-selective compounds. See FIG. 2.

### Biological Example 6

### pAKT (s473) ELISA Assay

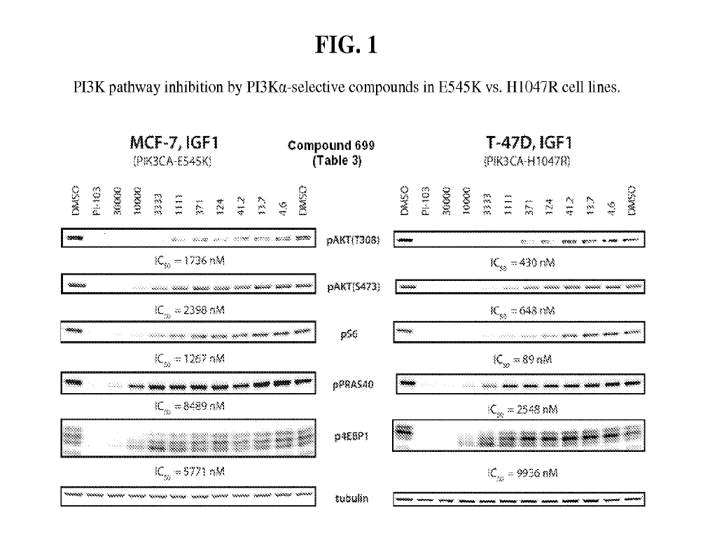

Cells were seeded onto 96-well plates (Corning, 3904) in their respective growth media at the densities listed in Table 5. Cells were incubated at 37° C., 5% CO₂ for 48 h (except MDA-MB-453 cells which were cultured in the absence of CO₂). Compounds were serially diluted in DMSO and subsequently diluted in serum-free DMEM. Test compounds were added to cells in serum-free DMEM at a final concentration of 0.3% DMSO and incubated for 3 h. Cells were then stimulated with growth factors as listed in Table 5. (For assays done in the presence of 10% FBS, compounds were added to cells at 10% FBS final concentration, cells were incubated for 3 hr, and no additional growth factors were added.) To fix the cells, medium was removed and 50 μL/well of 4% formaldehyde (Sigma, F8775) in high-salt TBS (20 mM Tris, pH 7.4; 500 mM NaCl) was added to each well at RT for 30 min. Cells were washed 3 times with 200 μL high-salt TBST (20 mM Tris, pH 7.4; 500 mM NaCl; 0.1% Triton X-100) and blocked with Odyssey blocking agent (Li-cor Biosciences, 927-40000) for 1 h. Anti-pAKT(S473) antibody (1:400 dilution factor, Cell Signaling Technology, 4060) and anti-total-AKT antibody (1:600 dilution factor, R&D Systems, MAB2055) were diluted in Odyssey blocking solution. 50 μL of antibody solution was added per well. After incubation overnight at 4° C., plates were washed 4 times with 200 μL high-salt TBST. Goat anti-rabbit IRDye 800CW- and goat anti-mouse IRDye 680-conjugated secondary antibodies (Li-cor Biosciences, 926-32211 and, 926-32220, respectively) diluted 1:400 in Odyssey block containing 0.1% Tween-20 were added as 50 μL to each well for 2 h at RT. Plates were washed 3 times with 200 μL high-salt TBST and 3 times with 200 μL high-salt TBS and then read on a Li-Cor Odyssey Infrared Imager with In-cell Western plug-in. Integrated intensities for pAKT(S473) were normalized to the signal for total AKT and IC₅₀ values were determined relative to the DMSO-treated control. See FIG. 1. In order to determine if PI3Kα mutation status correlated with increased sensitivity of AKT(T308) to isozyme selective inhibitors, two representative PI3Kα/mTOR dual and two representative PI3Kα selective compounds were tested for inhibition of growth factor mediated AKT(T308) phosphorylation in a wild type PI3Kα line (SK-BR-3), an additional E545K line (NCI-H460), and three additional H1047R lines (BT-20, HCT 116, and MDA-MB-453). pAKT(T308) IC₅₀ values for PI3Kα inhibitors were significantly lower in the four kinase domain mutant lines suggesting that the H1047R mutation may sensitize tumor cells to inhibition of PI3K signaling by PI3Kα-selective compounds.

The results were extended to monitor basal AKT phosphorylation in 10% FCS using pAKT(S473) as a more sensitive readout. IC₅₀ values for PI3Kα selective compounds were again lower in H1047R cells (T-47D) compared to E545K cells (MCF-7 and NCI-H460) or PI3Kα wild type cells (SK-BR-3 or A549). Differences in IC₅₀ values among cell lines were less pronounced for basal AKT phosphorylation in 10% FCS than for AKT activation in response to acute IGF1 stimulation.

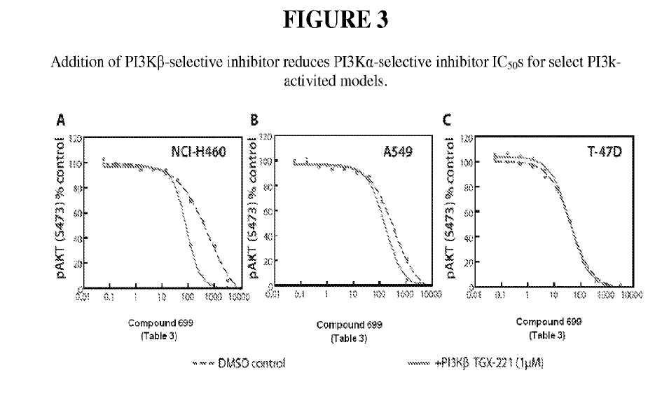

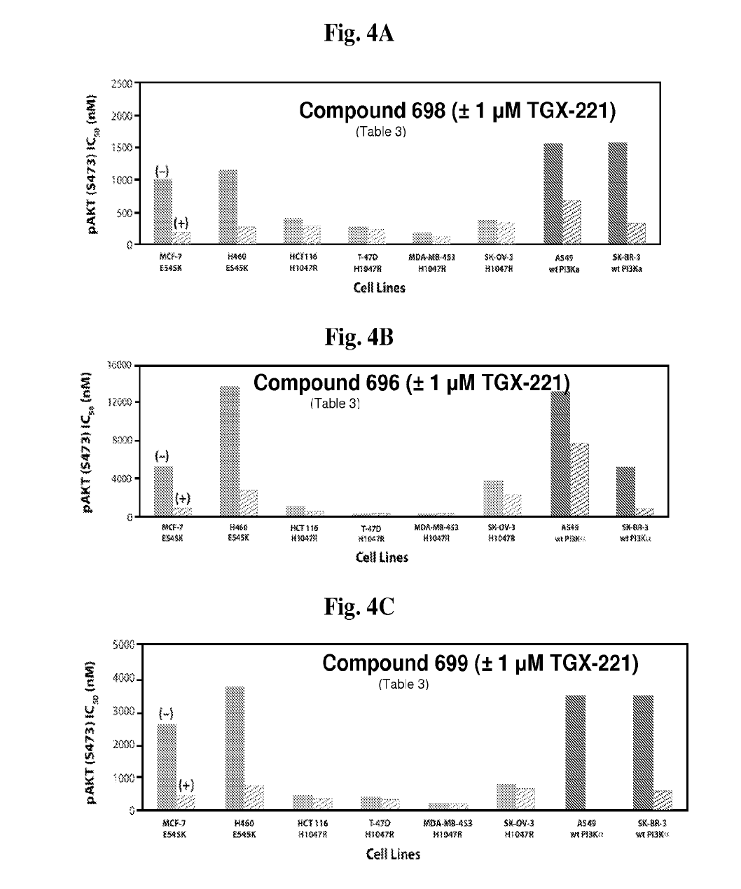

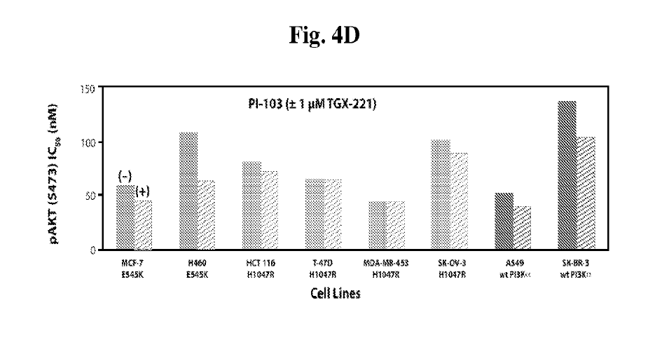

Results: A series of PI3K inhibitors with varying isozyme selectivity were profiled for inhibition of IGF1-mediated AKT(T308) phosophorylation in MCF-7 cells (PI3Kα-E545K mutant) and T-47D cells (PI3Kα-H1047R mutant) (Table 7). Both pan-PI3K/mTOR inhibitors and all four PI3Kα/mTOR dual inhibitors tested showed comparable IC₅₀ values in MCF-7 and T-47D cells. By contrast, all PI3Kα-selective compounds tested returned significantly lower IC50 values in T-47D than in MCF-7 cells (5-20 fold). PI3Kα-selective compounds also more potently inhibited phosphorylation of the downstream substrate RPS6 in T-47D vs. MCF-7 cells. For differential cellular activity of PI3Kα-selective compounds v. PI3Kα/mTOR vs. wild type, in different cell lines, please refer to FIGS. 1-4D and Tables 6 and 7.

Cell proliferation assays were also done in a panel of seven tumor cell lines using PI3Kα (H1047R) selective inhibitors, dual PI3Kα/mTOR selective inhibitors and a PI3Kβ selective inhibitor. IC₅₀ values for PI3Kα selective compounds were lowest in PI3K-H1047R/KRAS-wt lines T-47D, MDA-MB-453, HCT 116 (PI3K-H1047R/KRAS-G12D) and SK-OV-3, and highest in, (E545K) cell lines MCF-7 and, NCI-H460 (PI3K-E545K/KRAS-Q61H), and PI3Kα wild-type SK-BR-3. See Table 7. The results suggest that the PI3K-H1047R mutation may sensitize cells to the phenotypic effects of PI3Kα selective compounds while PTEN loss or KRAS activation may desensitize cells to these inhibitors. In these cell assays, as would be expected TGX-221 PI3Kβ inhibitor has no effect on PI3Kα mediated activity in these cell models.

TABLE 7
Differential cellular activity in PI3Kα-mutated models
Table 7. Differential cellular activity in PI3Kα-mutated models

| | Cellular pAKT (T308) IC₅₀ (nM) | | | | | | |
|---|---|---|---|---|---|---|---|
| | | | PI3KαH1047R | | | | |
| | PI3KαE545K | | | | MDA- | | PI3Kαwt |
| | | NCI- | HCT | T- | MB- | SK- | SK- |
| Com- | MCF-7 | H460 | 116 | 47D | 453 | OV-3 | BR-3 |
| pound | PI3Kα | mTOR | IGF1 | IGF1 | hereg | IGF1 | hereg |
| 698 | 2243 | 4698 | 462 | 258 | 118 | 438 | 2865 |
| 696 | 1649 | 8289 | 400 | 213 | 117 | 956 | 1240 |
| 699 | 2399 | 25551 | 719 | 207 | 639 | 1243 | 4540 |
| 693 | 120 | 523 | 168 | 73 | 40 | nd | 339 |
| 694 | 135 | 916 | 130 | 102 | 18 | nd | 115 |
| TGX-221 | >30000 | >30000 | 26447 | 27908 | >30000 | >30000 | >30000 |

### Biological Example 7-15

### Pharmacodynamic Xenograft Tumor Models

Female and male athymic nude mice (NCr) 5-8 weeks of age and weighing approximately 20-25 g are used in the following models. Prior to initiation of a study, the animals are allowed to acclimate for a minimum of 48 h. During these studies, animals are provided food and water ad libitum and housed in a room conditioned at 70-75° F. and 60% relative humidity. A 12 h light and 12 h dark cycle is maintained with automatic timers. All animals are examined daily for compound-induced or tumor-related deaths.

### MCF-7 Breast Adenocarcinoma Model

MCF7 human mammary adenocarcinoma cells are cultured in vitro in DMEM (Cellgro) supplemented with 10% Fetal Bovine Serum (Cellgro), Penicillin-Streptomycin and non-essential amino acids at 37° C. in a humidified 5% CO₂ atmosphere. On day 0, cells are harvested by trypsinization, and 5×10⁶ cells in 100 μL of a solution made of 50% cold Hanks balanced salt solution with 50% growth factor reduced matrigel (Becton Dickinson) implanted subcutaneously into the hindflank of female nude mice. A transponder is implanted into each mouse for identification and data tracking, and animals are monitored daily for clinical symptoms and survival.

Tumors are established in female athymic nude mice and staged when the average tumor weight reached 100-200 mg. A Compound of the Invention is orally administered as a solution/fine suspension in water (with 1:1 molar ratio of 1 N HCL) once-daily (qd) or twice-daily (bid) at 10, 25, 50 and 100 mg/kg for 14 days. During the dosing period of 14-19 days, tumor weights are determined twice-weekly and body weights are recorded daily.

### Colo-205 Colon Model

Colo-205 human colorectal carcinoma cells are cultured in vitro in DMEM (Mediatech) supplemented with 10% Fetal Bovine Serum (Hyclone), Penicillin-Streptomycin and non-essential amino acids at 37° C. in a humidified, 5% CO₂ atmosphere. On day 0, cells are harvested by trypsinization, and 3×10⁶ cells (passage 10-15, >95% viability) in 0.1 mL ice-cold Hank's balanced salt solution are implanted intradermally in the hind-flank of 5-8 week old female athymic nude mice. A transponder is implanted in each mouse for identification, and animals are monitored daily for clinical symptoms and survival.

Tumors are established in female athymic nude mice and staged when the average tumor weight reached 100-200 mg. A Compound of the Invention is orally administered as a solution/fine suspension in water (with 1:1 molar ratio of 1 N HCL) once-daily (qd) or twice-daily (bid) at 10, 25, 50 and 100 mg/kg for 14 days. During the dosing period of 14 days, tumor weights are determined twice-weekly and body weights are recorded daily.

### PC-3 Prostate Adenocarcinoma Model

PC-3 human prostate adenocarcinoma cells are cultured in vitro in DMEM (Mediatech) supplemented with 20% Fetal Bovine Serum (Hyclone), Penicillin-Streptomycin and non-essential amino acids at 37° C. in a humidified 5% CO₂ atmosphere. On day 0, cells are harvested by trypsinization and 3×10⁶ cells (passage 10-14, >95% viability) in 0.1 mL of ice-cold Hank's balanced salt solution are implanted subcutaneously into the hindflank of 5-8 week old male nude mice. A transponder is implanted in each mouse for identification, and animals are monitored daily for clinical symptoms and survival.

Tumors are established in male athymic nude mice and staged when the average tumor weight reached 100-200 mg. A Compound of the Invention is orally administered as a solution/fine suspension in water (with 1:1 molar ratio of 1 N HCl) once-daily (qd) or twice-daily (bid) at 10, 25, 50, or 100-mg/kg for 19 days. During the dosing period of 14-19 days, tumor weights are determined twice-weekly and body weights are recorded daily.

### U-87 MG Human Glioblastoma Model

U-87 MG human glioblastoma cells are cultured in vitro in DMEM (Mediatech) supplemented with 10% Fetal Bovine Serum (Hyclone), Penicillin-Streptomycin and non-essential amino acids at 37° C. in a humidified 5% CO₂ atmosphere. On day 0, cells are harvested by trypsinization and 2×10⁶ cells (passage 5, 96% viability) in 0.1 mL of ice-cold Hank's balanced salt solution are implanted intradermally into the hindflank of 5-8 week old female nude mice. A transponder is implanted in each mouse for identification, and animals are monitored daily for clinical symptoms and survival. Body weights are recorded daily.

### A549 Human Lung Carcinoma Model

A549 human lung carcinoma cells are cultured in vitro in DMEM (Mediatech) supplemented with 10% Fetal Bovine Serum (Hyclone), Penicillin-Streptomycin and non-essential amino acids at 37° C. in a humidified 5% CO₂ atmosphere. On day 0, cells are harvested by trypsinization and 10×10⁶ cells (passage 12, 99% viability) in 0.1 mL of ice-cold Hank's balanced salt solution are implanted intradermally into the hindflank of 5-8 week old female nude mice. A transponder is implanted in each mouse for identification, and animals are monitored daily for clinical symptoms and survival. Body weights are recorded daily.

### A2058 Human Melanoma Model

A2058 human melanoma cells are cultured in vitro in DMEM (Mediatech) supplemented with 10% Fetal Bovine Serum (Hyclone), Penicillin-Streptomycin and non-essential amino acids at 37° C. in a humidified, 5% CO₂ atmosphere. On day 0, cells are harvested by trypsinization and 3×10⁶ cells (passage 3, 95% viability) in 0.1 mL ice-cold Hank's balanced salt solution are implanted intradermally in the hind-flank of 5-8 week old female athymic nude mice. A transponder is implanted in each mouse for identification, and animals are monitored daily for clinical symptoms and survival. Body weights are recorded daily.

### WM-266-4 Human Melanoma Model

WM-266-4 human melanoma cells are cultured in vitro in DMEM (Mediatech) supplemented with 10% Fetal Bovine Serum (Hyclone), Penicillin-Streptomycin and non-essential amino acids at 37° C. in a humidified, 5% CO₂ atmosphere. On day 0, cells are harvested by trypsinization and 3×10⁶ cells (passage 5, 99% viability) in 0.1 mL ice-cold Hank's balanced salt solution are implanted intradermally in the hind-flank of 5-8 week old female athymic nude mice. A transponder is implanted in each mouse for identification, and animals are monitored daily for clinical symptoms and survival. Body weights are recorded daily.

Tumor weight (TW) in the above models is determined by measuring perpendicular diameters with a caliper, using the following formula:

tumor weight(mg)=[tumor volume=length(mm)×width²(mm²)]/2

These data were recorded and plotted on a tumor weight vs. days post-implantation line graph and presented graphically as an indication of tumor growth rates. Percent inhibition of tumor growth (TGI) is determined with the following formula:

$$[ 1 - ( \frac{( {X}_{f} - {X}_{0} )}{( {Y}_{f} - {X}_{0} )} ) ] * 100$$

where X₀=average TW of all tumors on group day

X_{f}=TW of treated group on Day f

Y_{f}=TW of vehicle control group on Day f

If tumors regress below their starting sizes, then the percent tumor regression is determined with the following formula:

$$( \frac{{X}_{0} - {X}_{f}}{{X}_{0}} ) * 100$$

Tumor size is calculated individually for each tumor to obtain a mean±SEM value for each experimental group. Statistical significance is determined using the 2-tailed Student's t-test (significance defined as P<0.05).

### BrdU Cell Proliferation Assay

Cells were seeded onto 96-well plates (Corning, 3904) in their respective growth media at the densities listed in Table 2. Cells were incubated at 37° C., 5% CO₂ overnight (except MDA-MB-453 cells which were cultured in the absence of CO₂). Cells were then treated the next day with a serial dilution of compound in their respective growth medium (containing a final concentration of 0.3% DMSO). Triplicate wells were used for each compound concentration. The control wells received 0.3% DMSO in growth medium. The plates were incubated at 37° C., 5% CO₂ for an additional 48 h. Cells were labeled with BrdU (Roche, 10280879001, 20 μM) for 2-4 h and then fixed with 70% EtOH+0.1 M NaOH for 30 min at RT. Anti-BrdU-Peroxidase (Roche, 11585860001, 1/2000 in PBS+1% BSA) conjugate was added to the cells, after which the plates were washed 3 times with 1×PBS. Chemiluminescent substrate solution (Pierce, 3707A and B) was added, and the plates were read for luminescence using the Wallac Victor plate reader for 0.1 sec. IC₅₀ values were determined based on cell proliferation with compound treatment compared to the 0.3% DMSO vehicle control.

### Illustrative Embodiments

A method for treating a subject having a tumor comprising:

(a) administering a PI3K-α selective inhibitor, a dual PI3K-α/mTOR selective inhibitor, or a combination of a PI3K-α selective inhibitor and a mTOR selective inhibitor to the subject if said tumor comprises a mutation in a PI3K-α kinase domain; or

(b) administering a combination of a PI3K-α selective inhibitor and a PI3K-β selective inhibitor, a dual PI3K-α/mTOR selective inhibitor, or a PI3K-β selective inhibitor, to said subject if said tumor comprises a mutation in a PI3K-α helical domain.

The method of embodiment 1, further comprising administering an additional chemotherapeutic agent in steps (a) or (b).

3. The method according to claim 2, wherein said additional chemotherapeutic agent comprises anti-microtubule agents; platinum coordination complexes; alkylating agents; antibiotic agents; topoisomerase II inhibitors; antimetabolites; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

4. The method of embodiment 2, wherein said additional therapeutic agent administered in step (b) further comprises a PI3K-δ selective inhibitor, a PI3K-γ selective inhibitor or a pan PI3K selective inhibitor.

5. The method according to embodiment 4, wherein said pan PI3K selective inhibitor comprises PI-103 or PIK-75.

6. The method according to embodiment 1, wherein the mutation in said kinase domain comprises a mutation at position 1047 of SEQ ID NO: 1.

7. The method according to embodiment 6, wherein said mutation of said kinase domain is a substitution of H1047R in SEQ ID NO: 1.

8. The method according to embodiment 1, wherein said mutation in said PI3K-α comprises a mutation in a helical domain.

9. The method according to embodiment 8, wherein said mutation in said helical domain comprises a mutation at position 545 in SEQ ID NO: 1.

10. The method according to embodiment 9, wherein said mutation at position 545 comprises a substitution of E545K in SEQ ID NO:1.

11. The method according to embodiment 1, wherein said PI3K-α selective inhibitor comprises a PI3K-α selective inhibitor selected from Table 1.

12. The method according to embodiment 11, wherein said PI3K-α selective inhibitor comprises compounds 696, 698, 699, 700, 701, 702, 703, 704, 705, 706 or 707.

13. The method according to embodiment 1, wherein said dual PI3K-α/mTOR selective inhibitor comprises a dual PI3K-α/mTOR selective inhibitor selected from Table I.

14. The method according to embodiment 13, wherein said dual PI3K-α/mTOR selective inhibitor comprises compounds 693, 694, 695 or 697.

15. The method according to embodiment i, wherein said combination of a PI3K-α selective inhibitor and a mTOR selective inhibitor comprises a PI3K-α selective inhibitor of Table 1 and a mTOR selective inhibitor of Table 1.

16. The method according to embodiment 1, wherein said mutation in said helical domain comprises a PI3K-α having a substitution E545K in SEQ ID NO: 1.

17. The method according to embodiment 1, wherein administering a PI3K-α selective inhibitor comprises administering a PI3K-α selective inhibitor to the subject in an amount varying from about 0.001 mg/kg to about 100 mg/kg.

18. The method according to embodiment 1, wherein administering a PK13K-β selective compound comprises administering a PKI3K-β selective inhibitor compound comprising TGX-221.

19. The method according to embodiment 18, wherein administering a PI3K-β selective inhibitor compound comprises administering the PI3K-β selective inhibitor compound to the subject in an amount ranging from about 0.001 mg/kg to about 100 mg/kg.

20. The method according to embodiment 1, wherein administering a dual PI3K-α/mTOR selective inhibitor comprises administering said dual PI3K-α/mTOR selective inhibitor to the subject in an amount ranging from about 0.001 mg/kg to about 100 mg/kg.

21. The method according to embodiment 1, wherein administering a combination of a PI3K-α selective inhibitor and a mTOR selective inhibitor compound comprises administering said combination of a PI3K-α selective inhibitor and a mTOR selective inhibitor to the subject, each inhibitor in an amount ranging from about 0.001 mg/kg to about 100 mg/kg.

22. The method according to any one of embodiments 1 to 21, wherein administering any one or more of a PI3K-α selective inhibitor, a PI3K-β selective inhibitor, a dual PI3K-α/mTOR selective inhibitor, a combination of a PI3K-α selective inhibitor and a mTOR selective inhibitor comprises administering said inhibitors or pharmaceutically acceptable salts thereof, in combination with a pharmaceutically acceptable carrier, excipient or diluent.

23. The method according to any one of embodiments 1 to 22, wherein said tumor is a breast cancer, a mantle cell lymphoma, a renal cell carcinoma, an acute myelogenous leukemia, a chronic myelogenous leukemia, a NPM/ALK-transformed anaplastic large cell lymphoma, a diffuse large B cell lymphoma, a rhabdomyosarcoma, an ovarian cancer, an endometrial cancer, a cervical cancer, a non-small cell lung carcinoma, a small-cell lung carcinoma, a melanoma, a prostate carcinoma, a thyroid carcinoma, an anaplastic large cell lymphoma, a hemangioma, a glioblastoma, or a head and neck cancer.

24. A method for identifying a selective inhibitor of a PI3K isozyme, the method comprising:

(a) contacting a first cell bearing a first mutation in a PI3K-α with a candidate inhibitor;

(b) contacting a second cell bearing a wild type PI3K-α, a PTEN null mutation, or a second mutation in said PI3K-α with the candidate inhibitor; and

(c) measuring AKT phosphorylation in said first and said second cells, wherein decreased AKT phosphorylation in said first cell when compared to said second cell identifies said candidate inhibitor as a selective PI3K-α inhibitor.

The method according to embodiment 24, wherein said first mutation in said PI3K-α comprises a mutation in a kinase domain of said PI3K-α.

26. The method according to embodiment 25, wherein said mutation in said kinase domain comprises a substitution at amino acid 1047 of SEQ ID NO:1.

27. The method according to embodiment 26, wherein said substituted amino acid at 1047 of SEQ ID NO: 1 is arginine in place of histidine.

28. The method according to embodiment 24, wherein said second mutation comprises a mutation in a helical domain of said PI3K-α.

29. The method according to embodiment 28, wherein said mutation in said helical domain comprises a substitution at amino acid 545 of SEQ ID NO:1.

30. The method according to embodiment 29, wherein said substituted amino acid at 545 of SEQ ID NO: 1 is lysine in place of glutamic acid.

31. The method according to embodiment 24, wherein said first cell comprises a cell from a cell line comprising HCT-116, T-47D, MDA-MB-453, SIGOV-3, BT-20 or LS H74T.

32. The method according to embodiment 24, wherein said second cell comprises a cell from a cell line comprising MCF-7, PC3 MCI-H460, SK-BR-3, PC-3, MDA-MB-468, SK-BR-3, MDA-MB-231T, or A549.

33. The method according to embodiment 24, further comprising adding a growth factor to said first and said second cells.

34. The method according to embodiment 33, wherein said growth factor comprises adding at least one of VEGF, IGF and heregulin to said first and said second cells.

35. The method according to embodiment 24, wherein measuring AKT phosphorylation in said first cell and said second cell comprises measuring an amount of AKT phosphorylation at a residue of AKT comprising T308, S473, S240/244 or combinations thereof.

36. The method according to embodiment 35, further comprising measuring the total amount of AKT present in said first and said second cells.

37. The method according to embodiment 35, wherein measuring said amount of AKT phosphorylation comprises adding an antibody specific for phosphorylated AKT and measuring binding of the antibody to AKT and determining said amount of phosphorylated AKT in the presence and absence of said candidate inhibitor.

38. The method according to embodiment 24, wherein measuring said AKT phosphorylation comprises determining an AKT phosphorylation IC₅₀ concentration of said candidate inhibitor in said first and said second cells.

39. The method according to embodiment 38, wherein identifying said candidate inhibitor as a selective PI3K-α inhibitor comprises determining that said IC₅₀ concentration of said candidate inhibitor is less than 50% of the IC₅₀ of the second cell.

40. The method according to embodiment 38, wherein identifying said candidate inhibitor as a selective PI3K-α inhibitor comprises determining that said IC₅₀ concentration of said candidate inhibitor is less than 20% of the IC₅₀ of the second cell.

41. A method for determining a treatment regimen for a cancer patient having a tumor comprising a PI3K-α, the method comprising:

determining the presence or absence of a mutation in amino acids 1047 and/or 545 of said PI3K-α;

wherein if said PI3K-α has a mutation at position 1047, said method comprises administering to the cancer patient a therapeutically effective amount of a PI3K-α selective inhibitor compound, or a dual PI3K-α/mTOR selective inhibitor, or a combination of a PI3K-α selective inhibitor and a mTOR selective inhibitor; or

wherein if said PI3K-α has a mutation at position 545, said method comprises administering to the cancer patient a therapeutically effective amount of a combination of a PI3K-α selective inhibitor and a PI3K-β selective inhibitor, or a dual PI3K-α/mTOR selective inhibitor, or a combination of a PI3K-α selective inhibitor and a mTOR selective inhibitor.

The method according to embodiment 41, wherein determining the presence or absence of a mutation in amino acids 1047 and/or 545 of said PI3K-α comprises isolating a nucleic acid sample encoding said PI3K-α or isolating said PI3K-α or a fragment thereof from said tumor.

43. The method according to embodiment 42, wherein said tumor cell is obtained from a tumor or cancer comprising: breast cancer, mantle cell lymphoma, renal cell carcinoma, acute myelogenous leukemia, chronic myelogenous leukemia, NPM/ALK-transformed anaplastic large cell lymphoma, diffuse large B cell lymphoma, rhabdomyosarcoma, ovarian cancer, endometrial cancer, cervical cancer, non-small cell lung carcinoma, small cell lung carcinoma, adenocarcinoma, colon cancer, rectal cancer, gastric carcinoma, hepatocellular carcinoma, melanoma, pancreatic cancer, prostate carcinoma, thyroid carcinoma, anaplastic large cell lymphoma, hemangioma, glioblastoma, or head and neck cancer.

44. The method according to embodiment 41, wherein said assay comprises whole genome sequencing, partial genome sequencing, exome sequencing, nucleic acid probe hybridization, restriction enzyme digestion analysis, direct sequencing, immunoprecipitation, western blotting or combinations thereof.

45. The method according to embodiment 41, wherein conducting an assay on said cell to determine the presence or absence of a mutation in amino acids 1047 and/or 545 of SEQ ID NO: 1 comprises extracting a nucleic acid comprising genomic DNA, total RNA or mRNA from said cell.

46. The method according to embodiment 45, wherein genomic DNA is used in said assay and said assay further comprises:

(a) amplifying a predetermined region of said genomic DNA;

(b) sequencing said amplified region to obtain a polynucleotide sequence of said amplified region; and

(c) determining whether said amplified region contains either a genetic mutation corresponding to position 1047 of the amino acid sequence of SEQ ID NO: 1, or a genetic mutation corresponding to position 545 of the amino acid sequence of SEQ ID NO: 1.

The method of embodiment 46, wherein amplifying a predetermined region of said genomic DNA comprises amplifying said genomic DNA using a pair of nucleic acid primers, a first primer capable of hybridizing stringently to a genomic DNA sequence upstream of a DNA codon encoding the amino acid at either 1047 or 545 of SEQ ID NO: 1, and second a nucleic acid primer operable to hybridize stringently to a genomic DNA sequence downstream of a DNA codon encoding the amino acid of either amino acid at 1047 or 545 of SEQ ID NO: 1.

48. The method according to embodiment 45, wherein said nucleic acid is an RNA sample.

49. The method according to embodiment 48, wherein said RNA sample is used in said assay, and said assay further comprises:

(a) reverse transcribing said RNA sample into an equivalent cDNA;

(b) amplifying a predetermined region of said cDNA using a pair of nucleic acid probes directed to a predetermined region of the PI3K-α gene;

(c) sequencing said amplified cDNA region to obtain a polynucleotide sequence of said amplified cDNA region; and

(d) determining whether said amplified cDNA region contains a gene mutation in a codon encoding the amino acid at either position 1047 and/or 545 of SEQ ID NO: 1.

The method according to embodiment 49, wherein amplifying a predetermined region of the cDNA comprises amplifying said cDNA using a pair of nucleic acid primers, a first primer capable of hybridizing stringently to said cDNA upstream of a DNA codon encoding the amino acid at either amino acid 1047 or 545 of SEQ ID NO: 1, and second a nucleic acid primer operable to hybridize stringently to said cDNA downstream of a DNA codon encoding the amino acid at either amino acid 1047 or 545 of SEQ ID NO: 1.

51. The method according to embodiment 50, wherein determining whether the amplified cDNA region contains a gene mutation comprises determining the presence or absence of a polynucleotide substitution of at least one nucleotide at position 3296, 3297 and 3298 of SEQ ID NO:2, wherein said substitution in the codon does not result in the codon encoding histidine; or determining the presence or absence of a polynucleotide substitution of at least one nucleotide at position 1790, 1791, and 1792 of SEQ ID NO:2, wherein the substitution in the codon does not result in the codon encoding glutamic acid.

52. The method according to embodiment 51, wherein the mutation at said codon at positions 3296, 3297 and 3298 of SEQ ID NO:2 results in the substituted codon encoding arginine at position 1047 of SEQ ID NO: 1.

53. The method according to embodiment 51, wherein the mutation at codon at positions position 1790, 1791, and 1792 of SEQ ID NO:2 results in the substituted codon encoding lysine at position 545 of SEQ ID NO:2.

54. The method according to embodiment 41, wherein said PI3K-α selective inhibitor comprises a PI3K-α selective inhibitor selected from Table 1.

55. The method according to embodiment 41, wherein said PI3K-α selective inhibitor comprises compounds 696, 698, 699, 700, 701, 702, 703, 704, 705, 706 or 707.

56. The method according to embodiment 41, wherein said dual PI3K-α/mTOR selective inhibitor comprises a dual PI3K-α/mTOR selective inhibitor selected from Table 1.

57. The method according to embodiment 13, wherein said dual PI3K-α/mTOR selective inhibitor comprises compounds 693, 694, 695 or 697.

58. The method according to embodiment 41, wherein said combination of a PI3K-α selective inhibitor and a mTOR selective inhibitor comprises a PI3K-α selective inhibitor of Table 1 and a mTOR selective inhibitor of Table 1.

59. The method according to embodiment 41, wherein said PI3K-β selective compound comprises TGX-221.

60. A method for inhibiting AKT activity in a cancer cell, the method comprising administering a therapeutically effective amount of at least one of: a PI3K-α selective inhibitor, a dual PI3K-α/mTOR selective inhibitor, and a combination of a PI3K-α selective inhibitor and a mTOR selective inhibitor to said cancer cell, wherein said cancer cell has a mutation in a kinase domain of PI3K-α.

61. The method according to embodiment 1, wherein said PI3K-α selective inhibitor comprises a PI3K-α selective inhibitor selected from Table 1.

62. The method according to embodiment 61, wherein said PI3K-α selective inhibitor comprises compounds 696, 698, 699, 700, 701, 702, 703, 704, 705, 706 or 707.

63. The method according to embodiment 60, wherein said dual PI3K-α/mTOR selective inhibitor comprises a dual PI3K-α/mTOR selective inhibitor selected from Table 1.

64. The method according to embodiment 63, wherein said dual PI3K-α mTOR selective inhibitor comprises compounds 693, 694, 695 or 697.

65. The method according to embodiment 60, wherein said combination of a PI3K-α selective inhibitor and a mTOR selective inhibitor comprises a PI3K-α selective inhibitor of Table 1 and a mTOR selective inhibitor of Table 1.

66. The method according to any one of embodiments 60-65, wherein said mutation in said kinase domain comprises a mutation in said PI3K-α having the substitution H1047R in SEQ ID NO:1.

67. A method for inhibiting proliferation of a cancer cell bearing a mutated PI3K-α, the method comprising administering a therapeutically effective amount of at least one of: a PI3K-α selective inhibitor, a dual PI3K-α/mTOR selective inhibitor, and a combination of a PI3K-α selective inhibitor and a mTOR selective inhibitor to said cancer cell, wherein said cancer cell has a mutation in a kinase domain of PI3K-α.

68. The method according to embodiment 67, wherein said PI3K-α selective inhibitor comprises a PI3K-α selective inhibitor selected from Table 1.

69. The method according to embodiment 67, wherein said PI3K-α selective inhibitor comprises compounds 696, 698, 699, 700, 701, 702, 703, 704, 705, 706 or 707.

70. The method according to embodiment 67, wherein said dual PI3K-α/mTOR selective inhibitor comprises a dual PI3K-α/mTOR selective inhibitor selected from Table 1.

71. The method according to embodiment 67, wherein said dual PI3K-α/mTOR selective inhibitor comprises compounds 693, 694, 695 or 697.

72. The method according to embodiment 67, wherein said combination of a PI3K-α selective inhibitor and a mTOR selective inhibitor comprises a PI3K-α selective inhibitor of Table 1 and a mTOR selective inhibitor of Table 1.

73. The method according to any one of embodiments 67-72, wherein said mutation in said kinase domain comprises a mutation in said PI3K-α having the substitution H1047R in SEQ ID NO:1.

74. A method for inhibiting PI3K-α activity in a cancer cell bearing a mutated PI3K-α, the method comprising administering a therapeutically effective amount of at least one of: a PI3K-α selective inhibitor, a dual PI3K-α/mTOR selective inhibitor, and a combination of a PI3K-α selective inhibitor and a mTOR selective inhibitor to said cancer cell, wherein said cancer cell has a mutation in a kinase domain of PI3K-α.

75. The method according to embodiment 74, wherein said PI3K-α selective inhibitor comprises a PI3K-α selective inhibitor selected from Table 1.

76. The method according to embodiment 74, wherein said PI3K-α selective inhibitor comprises compounds 696, 698, 699, 700, 701, 702, 703, 704, 705, 706 or 707.

77. The method according to embodiment 74, wherein said dual PI3K-α/mTOR selective inhibitor comprises a dual PI3K-α/mTOR selective inhibitor selected from Table 1.

78. The method according to embodiment 74, wherein said dual PI3K-α/mTOR selective inhibitor comprises compounds 693, 694, 695 or 697.

79. The method according to embodiment 74, wherein said combination of a PI3K-α selective inhibitor and a mTOR selective inhibitor comprises a PI3K-α selective inhibitor of Table I and a mTOR selective inhibitor of Table 1.

80. The method according to any one of embodiments 74-79 wherein said mutation in said kinase domain comprises a mutation in said PI3K-α having the substitution H1047R in SEQ ID NO:1.

81. A diagnostic kit for determining the suitability of administering a selective P3IK-α inhibitor to a cancer patient, said kit comprising:

(a) a receptacle, operable to receive a patient sample;

(b) one or more PI3K-α amino acid sequence determining reagents; and

(c) a set of instructions to assist in sequencing of said PI3K-α in a patient's sample for determining the presence or absence of a mutation in said PI3K-α.

The foregoing invention has been described in some detail by way of illustration and example, for purposes of clarity and understanding. The invention has been described with reference to various specific embodiments and techniques. However, it should be understood that many variations and modifications may be made while remaining within the spirit and scope of the invention. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled. All patents, patent applications and publications cited in this application are hereby incorporated by reference in their entirety for all purposes to the same extent as if each individual patent, patent application or publication were so individually denoted.

1. A method for treating a subject having a tumor comprising:
(a) administering a PI3K-α selective inhibitor, a dual PI3K-α/mTOR selective inhibitor, or a combination of a PI3K-α selective inhibitor and a mTOR selective inhibitor to the subject if said tumor comprises a mutation in a PI3K-α kinase domain; or
(b) administering a combination of a PI3K-α selective inhibitor and a PI3K-β selective inhibitor, a dual PI3K-α/mTOR selective inhibitor, or a PI3K-β selective inhibitor, to said subject if said tumor comprises a mutation in a PI3K-α helical domain.

2-81. (canceled)

82. The method of claim 1, further comprising administering an additional chemotherapeutic agent in steps (a) or (b), wherein said additional chemotherapeutic agent comprises anti-microtubule agents; platinum coordination complexes; alkylating agents; antibiotic agents; topoisomerase II inhibitors; antimetabolites; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

83. The method of claim 82, wherein said additional therapeutic agent administered in step (b) further comprises a PI3K-δ selective inhibitor, a PI3K-γ selective inhibitor or a pan PI3K selective inhibitor, wherein said pan PI3K selective inhibitor comprises PI-103 or PIK-75.

84. The method according to claim 1, wherein said mutation in said PI3K-α comprises a mutation in a kinase domain; wherein the mutation in said kinase domain comprises a mutation at position 1047 of SEQ ID NO: 1; and wherein said mutation of said kinase domain is a substitution of H1047R in SEQ ID NO: 1.

85. The method according to claim 1, wherein said mutation in said PI3K-α comprises a mutation in a helical domain; wherein said mutation in said helical domain comprises a mutation at position 542 or 545 in SEQ ID NO:1; and wherein said mutation at position 545 comprises a substitution of E542K or E545K in SEQ ID NO:1.

86. The method according to claim 1, wherein said PI3K-α selective inhibitor comprises a PI3K-α selective inhibitor selected from Table 1;
wherein said dual PI3K-α/mTOR selective inhibitor comprises a dual PI3K-α/mTOR selective inhibitor selected from Table 1; and wherein said combination of a PI3K-α selective inhibitor and a mTOR selective inhibitor comprises a PI3K-α selective inhibitor of Table 1 and a mTOR selective inhibitor of Table 1.

87. The method according to claim 86, wherein said PI3K-α selective inhibitor comprises a PI3K-α selective inhibitor selected from the group consisting of 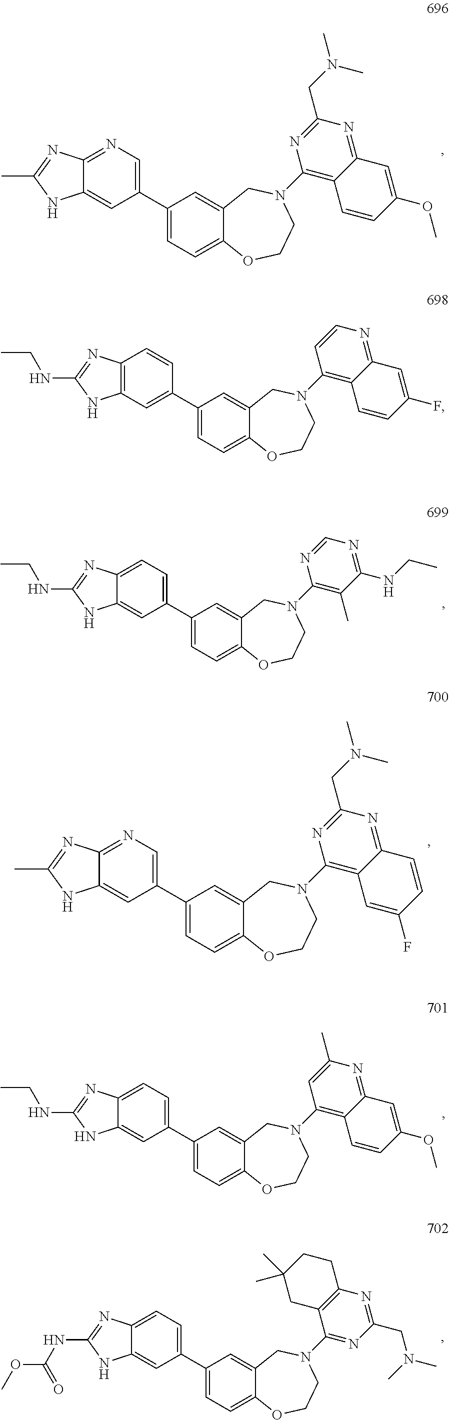 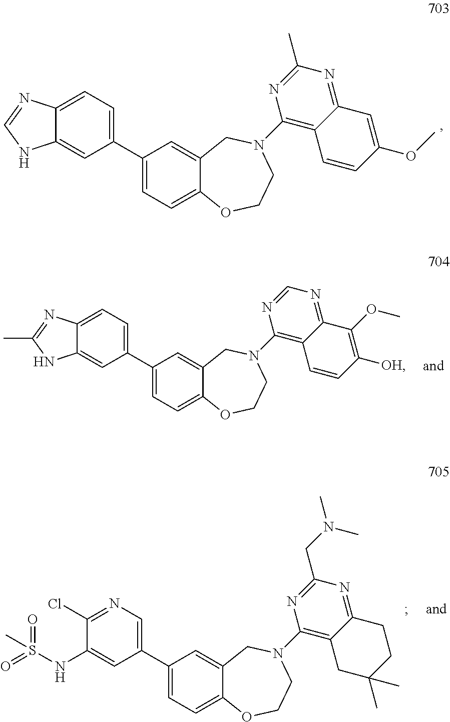
wherein said dual PI3K-α/mTOR selective inhibitor comprises a compound selected from the group consisting of

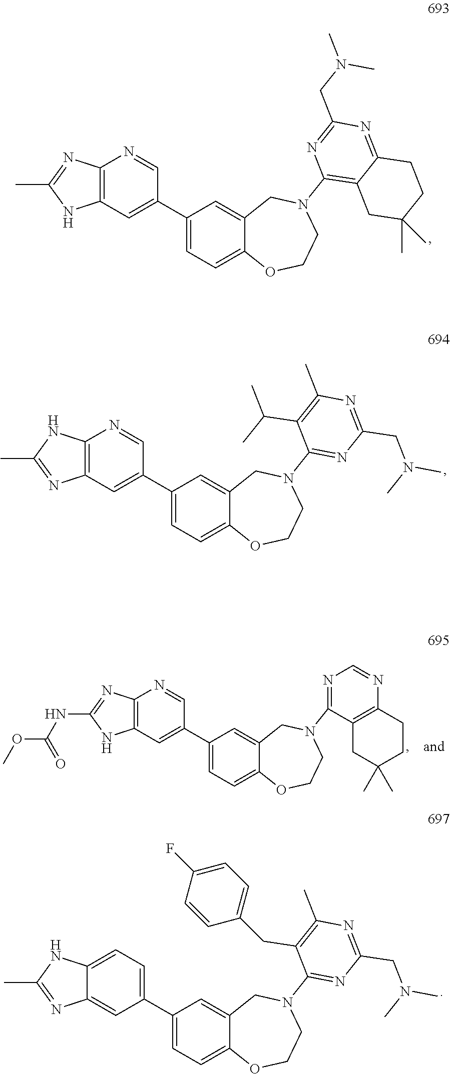

88. The method according to claim 1, wherein administering a PK13K-β selective compound comprises administering a PKI3K-β selective inhibitor compound comprising TGX-221.

89. The method according to claim 1, wherein said administering any one or more of a PI3K-α selective inhibitor, a PI3K-β selective inhibitor, a dual PI3K-α/mTOR selective inhibitor, a combination of a PI3K-α selective inhibitor and a mTOR selective inhibitor further comprises administering said inhibitors or pharmaceutically acceptable salts thereof, in combination with a pharmaceutically acceptable carrier, excipient or diluent.

90. The method according to claim 1, wherein said tumor is a breast cancer, a mantle cell lymphoma, a renal cell carcinoma, an acute myelogenous leukemia, a chronic myelogenous leukemia, a NPM/ALK-transformed anaplastic large cell lymphoma, a diffuse large B cell lymphoma, a rhabdomyosarcoma, an ovarian cancer, an endometrial cancer, a cervical cancer, a non-small cell lung carcinoma, a small-cell lung carcinoma, a melanoma, a pancreatic cancer, a prostate carcinoma, a thyroid carcinoma, an anaplastic large cell lymphoma, a hemangioma, a glioblastoma, or a head and neck cancer.

91. The method according to claim 1, wherein said tumor comprises a mutation in a PI3K-α kinase domain, the method further comprising inhibiting AKT activity in a cancer cell.

92. The method according to claim 1, wherein said tumor comprises a mutation in a PI3K-α kinase domain, the method further comprising inhibiting proliferation of a cancer cell bearing a mutated PI3K-α.

93. The method according to claim 1, wherein said tumor comprises a mutation in a PI3K-α kinase domain, the method further comprising inhibiting PI3K-α activity in a cancer cell bearing a mutated PI3K-α

94. A method for identifying a selective inhibitor of a PI3K isozyme, the method comprising:
(a) contacting a first cell bearing a mutation in a kinase domain of a PI3K-α with a candidate inhibitor;
(b) contacting a second cell bearing a wild type PI3K-α, a PTEN null mutation, or a mutation in a helical domain of said PI3K-α with the candidate inhibitor; and
(c) measuring AKT phosphorylation in said first and said second cells, wherein decreased AKT phosphorylation in said first cell when compared to said second cell identifies said candidate inhibitor as a selective PI3K-α inhibitor.

95. The method according to claim 94, wherein said mutation in said kinase domain comprises a substitution at amino acid 1047 of SEQ ID NO: 1;
wherein said substituted amino acid at 1047 of SEQ ID NO: 1 is arginine in place of histidine;
wherein said mutation in said helical domain comprises a substitution at amino acid 542 or 545 of SEQ ID NO:1; and
wherein said substituted amino acid at 542 or 545 of SEQ ID NO: 1 is lysine in place of glutamic acid.

96. The method according to claim 94, wherein said first cell comprises a cell from a cell line comprising HCT-116, T-47D, MDA-MB-453, SIGOV-3, BT-20 or LS H74T; and
wherein said second cell comprises a cell from a cell line comprising MCF-7, PC3 MCI-H460, SK-BR-3, PC-3, MDA-MB-468, SK-BR-3, MDA-MB-231T, or A549.

97. The method according to claim 94, further comprising adding a growth factor to said first and said second cells, wherein said growth factor comprises adding at least one of VEGF, IGF and heregulin to said first and said second cells.

98. The method according to claim 94, wherein measuring AKT phosphorylation in said first cell and said second cell comprises measuring an amount of AKT phosphorylation at a residue of AKT comprising T308, S473, S240/244 or combinations thereof;
wherein said method further comprises measuring the total amount of AKT present in said first and said second cells;
wherein measuring said amount of AKT phosphorylation comprises adding an antibody specific for phosphorylated AKT and measuring binding of the antibody to AKT and determining said amount of phosphorylated AKT in the presence and absence of said candidate inhibitor; and
wherein measuring said AKT phosphorylation comprises determining an AKT phosphorylation IC₅₀ concentration of said candidate inhibitor in said first and said second cells.

99. The method according to claim 98, wherein identifying said candidate inhibitor as a selective PI3K-α inhibitor comprises determining that said IC₅₀ concentration of said candidate inhibitor is less than 50% of the IC₅₀ of the second cell.

100. A method for determining a treatment regimen for a cancer patient having a tumor comprising a PI3K-α, the method comprising:
determining the presence or absence of a mutation in amino acids 1047 and/or 545 of said PI3K-α;
wherein if said PI3K-α has a mutation at position 1047, said method comprises administering to the cancer patient a therapeutically effective amount of a PI3K-α selective inhibitor compound, or a dual PI3K-α/mTOR selective inhibitor, or a combination of a PI3K-α selective inhibitor and a mTOR selective inhibitor; or
wherein if said PI3K-α has a mutation at position 545, said method comprises administering to the cancer patient a therapeutically effective amount of a combination of a PI3K-α selective inhibitor and a PI3K-β selective inhibitor, or a dual PI3K-α/mTOR selective inhibitor, or a combination of a PI3K-α selective inhibitor and a mTOR selective inhibitor.

101. The method according to claim 100, wherein determining the presence or absence of a mutation in amino acids 1047 and/or 545 of said PI3K-α comprises isolating a nucleic acid sample encoding said PI3K-α or isolating said PI3K-α or a fragment thereof from said tumor.

102. The method according to claim 100, wherein said tumor cell is obtained from a tumor or cancer comprising: breast cancer, mantle cell lymphoma, renal cell carcinoma, acute myelogenous leukemia, chronic myelogenous leukemia, NPM/ALK-transformed anaplastic large cell lymphoma, diffuse large B cell lymphoma, rhabdomyosarcoma, ovarian cancer, endometrial cancer, cervical cancer, non-small cell lung carcinoma, small cell lung carcinoma, adenocarcinoma, colon cancer, rectal cancer, gastric carcinoma, hepatocellular carcinoma, melanoma, pancreatic cancer, prostate carcinoma, thyroid carcinoma, anaplastic large cell lymphoma, hemangioma, glioblastoma, or head and neck cancer.

103. The method according to claim 100, wherein said determining the presence or absence of a mutation in amino acids 1047 and/or 545 of said PI3K-α comprises whole genome sequencing, partial genome sequencing, exome sequencing, nucleic acid probe hybridization, restriction enzyme digestion analysis, direct sequencing, immunoprecipitation, western blotting or combinations thereof.

104. The method according to claim 100, wherein said determining the presence or absence of a mutation in amino acids 1047 and/or 545 of said PI3K-α comprises extracting a nucleic acid comprising genomic DNA, total RNA or mRNA from said cell.

105. The method according to claim 104, wherein said nucleic acid comprises genomic DNA, wherein said method further comprises:
(a) amplifying a predetermined region of said genomic DNA;
(b) sequencing said amplified region to obtain a polynucleotide sequence of said amplified region; and
(c) determining whether said amplified region contains either a genetic mutation corresponding to position 1047 of the amino acid sequence of SEQ ID NO:1, or a genetic mutation corresponding to position 545 of the amino acid sequence of SEQ ID NO:1.

106. The method of claim 105, wherein amplifying a predetermined region of said genomic DNA comprises amplifying said genomic DNA using a pair of nucleic acid primers, a first primer capable of hybridizing stringently to a genomic DNA sequence upstream of a DNA codon encoding the amino acid at either 1047 or 545 of SEQ ID NO:1, and second a nucleic acid primer operable to hybridize stringently to a genomic DNA sequence downstream of a DNA codon encoding the amino acid of either amino acid at 1047 or 545 of SEQ ID NO: 1.

107. The method according to claim 104, wherein said nucleic acid comprises an RNA sample, wherein said method further comprises:
(a) reverse transcribing said RNA sample into an equivalent cDNA;
(b) amplifying a predetermined region of said cDNA using a pair of nucleic acid probes directed to a predetermined region of the PI3K-α gene;
(c) sequencing said amplified cDNA region to obtain a polynucleotide sequence of said amplified cDNA region; and
(d) determining whether said amplified cDNA region contains a gene mutation in a codon encoding the amino acid at position 1047, 542, or 545 of SEQ ID NO:1.

108. The method according to claim 107, wherein amplifying a predetermined region of the cDNA comprises amplifying said cDNA using a pair of nucleic acid primers, a first primer capable of hybridizing stringently to said cDNA upstream of a DNA codon encoding the amino acid at either amino acid 1047 or 542 or 545 of SEQ ID NO:1, and second a nucleic acid primer operable to hybridize stringently to said cDNA downstream of a DNA codon encoding the amino acid at either amino acid 1047 or 542 or 545 of SEQ ID NO:1;
wherein determining whether the amplified cDNA region contains a gene mutation comprises determining the presence or absence of a polynucleotide substitution of at least one nucleotide at position 3296, 3297 and 3298 of SEQ ID NO:2, wherein said substitution in the codon does not result in the codon encoding histidine; or determining the presence or absence of a polynucleotide substitution of at least one nucleotide at position 1790, 1791, and 1792 of SEQ ID NO:2, wherein the substitution in the codon does not result in the codon encoding glutamic acid;
wherein the mutation at said codon at positions 3296, 3297 and 3298 of SEQ ID NO:2 results in the substituted codon encoding arginine at position 1047 of SEQ ID NO: 1; and
wherein the mutation at codon at positions position 1790, 1791, and 1792 of SEQ ID NO:2 results in the substituted codon encoding lysine at position 545 of SEQ ID NO:2.

109. The method according to claim 100, wherein said PI3K-α selective inhibitor comprises a PI3K-α selective inhibitor selected from Table 1;
wherein said dual PI3K-α/mTOR selective inhibitor comprises a dual PI3K-α/mTOR selective inhibitor selected from Table 1; and
wherein said combination of a PI3K-α selective inhibitor and a mTOR selective inhibitor comprises a PI3K-α selective inhibitor of Table 1 and a mTOR selective inhibitor of Table 1.

110. The method according to claim 109, wherein said PI3K-α selective inhibitor comprises a compound selected from the group consisting of 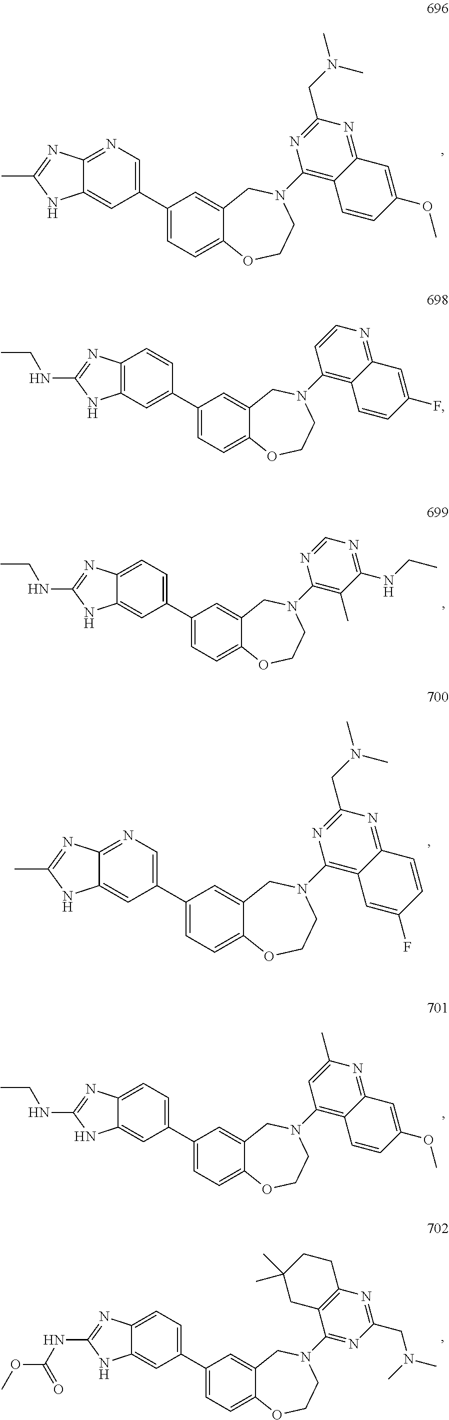 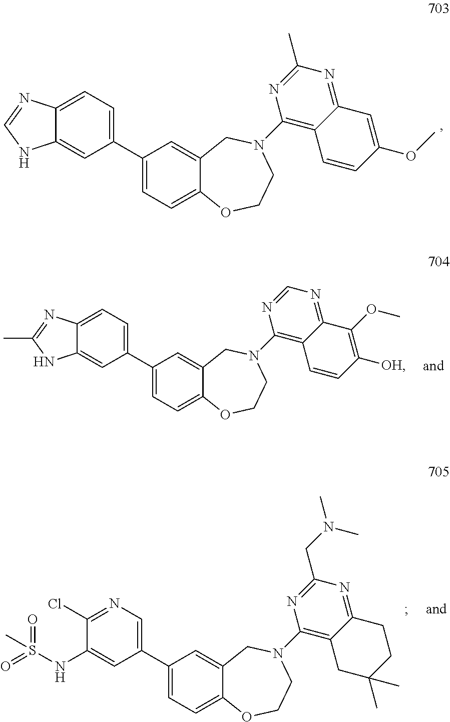
wherein said dual PI3K-α/mTOR selective inhibitor comprises a compound selected from the group consisting of

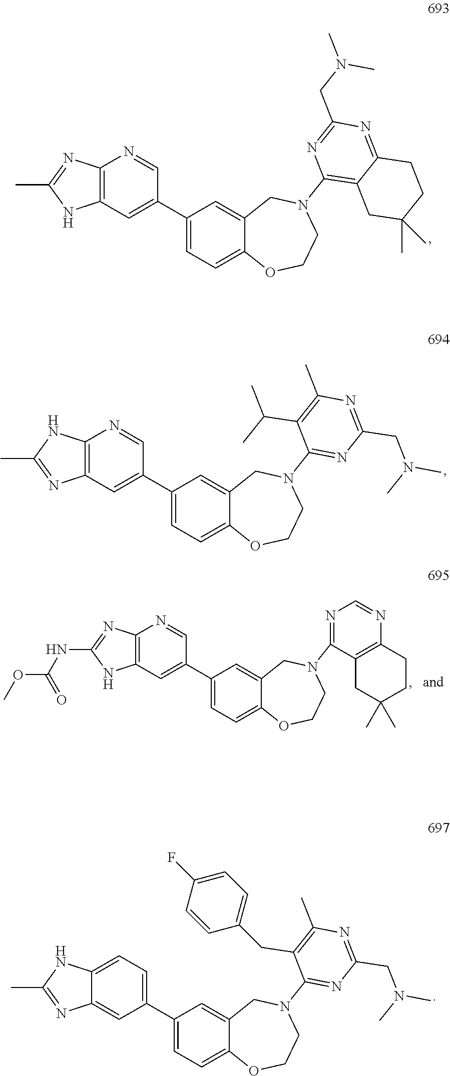

111. The method according to claim 100, wherein said PI3K-β selective compound comprises TGX-221.

112. A diagnostic kit for determining the suitability of administering a selective P3IK-α inhibitor to a cancer patient, said kit comprising:
(a) a receptacle, operable to receive a patient sample;
(b) one or more PI3K-α amino acid sequence determining reagents; and
(c) a set of instructions to assist in sequencing of said PI3K-α in a patient's sample for determining the presence or absence of a mutation in said PI3K-α.


SEQUENCE LISTING (2 sequences)

SEQ ID NO: 1 - protein, 1068 aa, Homo sapiens
  MISC_FEATURE: Human phosphatidylinositol 3-kinase catalytic subunit alpha polypeptide (PIK3CA) encoded by mRNA polynucleotide sequence ID NCBI Accession Reference No NM_006218; GI54792081 (3724 nucleotides)
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 2 - DNA, 3724 bp, Homo sapiens
  misc_feature: Human phosphatidylinositol 3-kinase catalytic subunit alpha mRNA polynucleotide sequence ID NCBI Accession Reference No NM_006218; GI54792081 (3724 nucleotides)
tctccctcggcgccgccgccgccgcccgcggggctgggacccgatgcggttagagccgcggagcctggaagagccccgagcgtttctgctttgggacaaccatacatctaattccttaaagtagttttatatgtaaaacttgcaaagaatcagaacaatgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaaaagataactgagaaaatgaaagctcactctggattccacactgcactgttaataactctcagcaggcaaagaccgattgcataggaattgcacaatccatgaacagcattagaatttacagcaagaacagaaataaaatactatataatttaaataatgtaaacgcaaacagggtttgatagcacttaaactagttcatttcaaaattaagctttagaataatgcgcaatttcatgttatgccttaagtccaaaaaggtaaactttgaagattgtttgtatctttttttaaaaaacaaaacaaaacaaaaatccccaaaatatatagaaatgatggagaaggaaaaaaaaaaaaaaaaa